SEQUENCE LISTING (2328 sequences)

SEQ ID NO: 1 - RNA, 21 nt, Homo sapiens
uggaauguaaagaaguaugua

SEQ ID NO: 2 - RNA, 22 nt, Homo sapiens
uggaauguaaggaagugugugg

SEQ ID NO: 3 - RNA, 22 nt, Homo sapiens
uggaagacuagugauuuuguug

SEQ ID NO: 4 - RNA, 23 nt, Homo sapiens
uacccuguagauccgaauuugug

SEQ ID NO: 5 - RNA, 22 nt, Homo sapiens
uaaggugcaucuagugcagaua

SEQ ID NO: 6 - RNA, 22 nt, Homo sapiens
uaaggugcaucuagugcaguua

SEQ ID NO: 7 - RNA, 21 nt, Homo sapiens
acugcccuaagugcuccuucu

SEQ ID NO: 8 - RNA, 23 nt, Homo sapiens
caaagugcucauagugcagguag

SEQ ID NO: 9 - RNA, 24 nt, Homo sapiens
aaaagugcuuacagugcagguagc

SEQ ID NO: 10 - RNA, 21 nt, Homo sapiens
uaaagugcugacagugcagau

SEQ ID NO: 11 - RNA, 22 nt, Homo sapiens
uggcucaguucagcaggaacag

SEQ ID NO: 12 - RNA, 22 nt, Homo sapiens
cauugcacuugucucggucuga

SEQ ID NO: 13 - RNA, 21 nt, Homo sapiens
uauugcacauuacuaaguugc

SEQ ID NO: 14 - RNA, 21 nt, Homo sapiens
uucaaguaauccaggauaggc

SEQ ID NO: 15 - RNA, 22 nt, Homo sapiens
uucaaguaauucaggauagguu

SEQ ID NO: 16 - RNA, 21 nt, Homo sapiens
uucacaguggcuaaguucugc

SEQ ID NO: 17 - RNA, 22 nt, Homo sapiens
aaggagcucacagucuauugag

SEQ ID NO: 18 - RNA, 21 nt, Homo sapiens
uagcaccaucugaaaucgguu

SEQ ID NO: 19 - RNA, 20 nt, Homo sapiens
uagcaccauuugaaaucggu

SEQ ID NO: 20 - RNA, 22 nt, Homo sapiens
uguaaacauccuacacucagcu

SEQ ID NO: 21 - RNA, 23 nt, Homo sapiens
uguaaacauccuacacucucagc

SEQ ID NO: 22 - RNA, 22 nt, Homo sapiens
aaagugcuguucgugcagguag

SEQ ID NO: 23 - RNA, 23 nt, Homo sapiens
uaagugcuuccauguuugagugu

SEQ ID NO: 24 - RNA, 22 nt, Homo sapiens
uucaacggguauuuauugagca

SEQ ID NO: 25 - RNA, 22 nt, Homo sapiens
ugagguaguaaguuguauuguu

SEQ ID NO: 26 - RNA, 22 nt, Homo sapiens
cagugcaauguuaaaagggcau

SEQ ID NO: 27 - RNA, 22 nt, Homo sapiens
cagugcaaugaugaaagggcau

SEQ ID NO: 28 - RNA, 23 nt, Homo sapiens
cagugcaauaguauugucaaagc

SEQ ID NO: 29 - RNA, 22 nt, Homo sapiens
uaacagucuacagccauggucg

SEQ ID NO: 30 - RNA, 21 nt, Homo sapiens
uaacagucuccagucacggcc

SEQ ID NO: 31 - RNA, 22 nt, Homo sapiens
uugguccccuucaaccagcugu

SEQ ID NO: 32 - RNA, 21 nt, Homo sapiens
uugguccccuucaaccagcua

SEQ ID NO: 33 - RNA, 21 nt, Homo sapiens
ugugacugguugaccagaggg

SEQ ID NO: 34 - RNA, 22 nt, Homo sapiens
uauugcuuaagaauacgcguag

SEQ ID NO: 35 - RNA, 23 nt, Homo sapiens
uguaguguuuccuacuuuaugga

SEQ ID NO: 36 - RNA, 22 nt, Homo sapiens
ugagaacugaauuccauggguu

SEQ ID NO: 37 - RNA, 20 nt, Homo sapiens
guguguggaaaugcuucugc

SEQ ID NO: 38 - RNA, 22 nt, Homo sapiens
ucuggcuccgugucuucacucc

SEQ ID NO: 39 - RNA, 22 nt, Homo sapiens
aacauucaaccugucggugagu

SEQ ID NO: 40 - RNA, 24 nt, Homo sapiens
aacauucauuguugucgguggguu

SEQ ID NO: 41 - RNA, 21 nt, Homo sapiens
ugguucuagacuugccaacua

SEQ ID NO: 42 - RNA, 23 nt, Homo sapiens
uauggcacugguagaauucacug

SEQ ID NO: 43 - RNA, 22 nt, Homo sapiens
uggacggagaacugauaagggu

SEQ ID NO: 44 - RNA, 21 nt, Homo sapiens
cugaccuaugaauugacagcc

SEQ ID NO: 45 - RNA, 21 nt, Homo sapiens
augaccuaugaauugacagac

SEQ ID NO: 46 - RNA, 21 nt, Homo sapiens
aacuggccuacaaagucccag

SEQ ID NO: 47 - RNA, 24 nt, Homo sapiens
aacuggcccucaaagucccgcuuu

SEQ ID NO: 48 - RNA, 21 nt, Homo sapiens
uagcagcacagaaauauuggc

SEQ ID NO: 49 - RNA, 22 nt, Homo sapiens
cagcagcaauucauguuuugaa

SEQ ID NO: 50 - RNA, 21 nt, Homo sapiens
cagcagcacacugugguuugu

SEQ ID NO: 51 - RNA, 21 nt, Homo sapiens
uagguaguuucauguuguugg

SEQ ID NO: 52 - RNA, 21 nt, Homo sapiens
uagguaguuuccuguuguugg

SEQ ID NO: 53 - RNA, 22 nt, Homo sapiens
uucaccaccuucuccacccagc

SEQ ID NO: 54 - RNA, 22 nt, Homo sapiens
uacaguagucugcacauugguu

SEQ ID NO: 55 - RNA, 22 nt, Homo sapiens
uaacacugucugguaacgaugu

SEQ ID NO: 56 - RNA, 22 nt, Homo sapiens
agagguauagggcaugggaaaa

SEQ ID NO: 57 - RNA, 22 nt, Homo sapiens
uucccuuugucauccuaugccu

SEQ ID NO: 58 - RNA, 22 nt, Homo sapiens
uucccuuugucauccuucgccu

SEQ ID NO: 59 - RNA, 22 nt, Homo sapiens
uccuucauuccaccggagucug

SEQ ID NO: 60 - RNA, 22 nt, Homo sapiens
auaagacgagcaaaaagcuugu

SEQ ID NO: 61 - RNA, 24 nt, Homo sapiens
agcuacaucuggcuacugggucuc

SEQ ID NO: 62 - RNA, 22 nt, Homo sapiens
uaugugggaugguaaaccgcuu

SEQ ID NO: 63 - RNA, 22 nt, Homo sapiens
ugguuuaccgucccacauacau

SEQ ID NO: 64 - RNA, 22 nt, Homo sapiens
uaaacguggauguacuugcuuu

SEQ ID NO: 65 - RNA, 23 nt, Homo sapiens
acuuuaacauggaagugcuuucu

SEQ ID NO: 66 - RNA, 22 nt, Homo sapiens
ccacugccccaggugcugcugg

SEQ ID NO: 67 - RNA, 23 nt, Homo sapiens
cgcauccccuagggcauuggugu

SEQ ID NO: 68 - RNA, 22 nt, Homo sapiens
aacacaccugguuaaccucuuu

SEQ ID NO: 69 - RNA, 21 nt, Homo sapiens
gccccugggccuauccuagaa

SEQ ID NO: 70 - RNA, 23 nt, Homo sapiens
uccagcuccuauaugaugccuuu

SEQ ID NO: 71 - RNA, 23 nt, Homo sapiens
uccgucucaguuacuuuauagcc

SEQ ID NO: 72 - RNA, 24 nt, Homo sapiens
ucucacacagaaaucgcacccguc

SEQ ID NO: 73 - RNA, 23 nt, Homo sapiens
ugucugcccgcaugccugccucu

SEQ ID NO: 74 - RNA, 24 nt, Homo sapiens
aauccuuggaaccuaggugugagu

SEQ ID NO: 75 - RNA, 22 nt, Homo sapiens
acauagaggaaauuccacguuu

SEQ ID NO: 76 - RNA, 21 nt, Homo sapiens
gugccgccaucuuuugagugu

SEQ ID NO: 77 - RNA, 21 nt, Homo sapiens
aucauagaggaaaauccacgu

SEQ ID NO: 78 - RNA, 22 nt, Homo sapiens
aucauagaggaaaauccauguu

SEQ ID NO: 79 - RNA, 22 nt, Homo sapiens
aucacacaaaggcaacuuuugu

SEQ ID NO: 80 - RNA, 22 nt, Homo sapiens
cuccugacuccagguccugugu

SEQ ID NO: 81 - RNA, 22 nt, Homo sapiens
ugguugaccauagaacaugcgc

SEQ ID NO: 82 - RNA, 22 nt, Homo sapiens
gaaguuguucgugguggauucg

SEQ ID NO: 83 - RNA, 23 nt, Homo sapiens
acuucaccugguccacuagccgu

SEQ ID NO: 84 - RNA, 22 nt, Homo sapiens
cuggacuuagggucagaaggcc

SEQ ID NO: 85 - RNA, 22 nt, Homo sapiens
cuggacuuggagucagaaggcc

SEQ ID NO: 86 - RNA, 22 nt, Homo sapiens
agcucggucugaggccccucag

SEQ ID NO: 87 - RNA, 21 nt, Homo sapiens
ugucuugcaggccgucaugca

SEQ ID NO: 88 - RNA, 21 nt, Homo sapiens
cuggauggcuccuccaugucu

SEQ ID NO: 89 - RNA, 22 nt, Homo sapiens
aucaugaugggcuccucggugu

SEQ ID NO: 90 - RNA, 22 nt, Homo sapiens
uggcaguguauuguuagcuggu

SEQ ID NO: 91 - RNA, 23 nt, Homo sapiens
aaaccguuaccauuacugaguuu

SEQ ID NO: 92 - RNA, 22 nt, Homo sapiens
uguuugcagaggaaacugagac

SEQ ID NO: 93 - RNA, 21 nt, Homo sapiens
ucagucucaucugcaaagaag

SEQ ID NO: 94 - RNA, 22 nt, Homo sapiens
uaugugccuuuggacuacaucg

SEQ ID NO: 95 - RNA, 23 nt, Homo sapiens
ucacuccucuccucccgucuucu

SEQ ID NO: 96 - RNA, 22 nt, Homo sapiens
agaggcuggccgugaugaauuc

SEQ ID NO: 97 - RNA, 21 nt, Homo sapiens
cccagauaauggcacucucaa

SEQ ID NO: 98 - RNA, 23 nt, Homo sapiens
agugacaucacauauacggcagc

SEQ ID NO: 99 - RNA, 23 nt, Homo sapiens
aguggggaacccuuccaugagga

SEQ ID NO: 100 - RNA, 22 nt, Homo sapiens
uuguacaugguaggcuuucauu

SEQ ID NO: 101 - RNA, 24 nt, Homo sapiens
ugaaacauacacgggaaaccucuu

SEQ ID NO: 102 - RNA, 22 nt, Homo sapiens
aauccuuugucccugggugaga

SEQ ID NO: 103 - RNA, 21 nt, Homo sapiens
agacccuggucugcacucuau

SEQ ID NO: 104 - RNA, 22 nt, Homo sapiens
gucaacacuugcugguuuccuc

SEQ ID NO: 105 - RNA, 21 nt, Homo sapiens
uaaggcacccuucugaguaga

SEQ ID NO: 106 - RNA, 21 nt, Homo sapiens
uuuugcaccuuuuggagugaa

SEQ ID NO: 107 - RNA, 23 nt, Homo sapiens
ugauugguacgucuguggguaga

SEQ ID NO: 108 - RNA, 23 nt, Homo sapiens
uacucaggagaguggcaaucaca

SEQ ID NO: 109 - RNA, 21 nt, Homo sapiens
gugucuuuugcucugcaguca

SEQ ID NO: 110 - RNA, 22 nt, Homo sapiens
aagugcugucauagcugagguc

SEQ ID NO: 111 - RNA, 23 nt, Homo sapiens
cacucagccuugagggcacuuuc

SEQ ID NO: 112 - RNA, 22 nt, Homo sapiens
uucacagggaggugucauuuau

SEQ ID NO: 113 - RNA, 23 nt, Homo sapiens
caucuggagguaagaagcacuuu

SEQ ID NO: 114 - RNA, 22 nt, Homo sapiens
ccucuagauggaagcacugucu

SEQ ID NO: 115 - RNA, 23 nt, Homo sapiens
aucgugcaucccuuuagaguguu

SEQ ID NO: 116 - RNA, 22 nt, Homo sapiens
aucgugcauccuuuuagagugu

SEQ ID NO: 117 - RNA, 22 nt, Homo sapiens
ucgugcaucccuuuagaguguu

SEQ ID NO: 118 - RNA, 22 nt, Homo sapiens
caaagcgcuccccuuuagaggu

SEQ ID NO: 119 - RNA, 22 nt, Homo sapiens
caaagcgcuucucuuuagagug

SEQ ID NO: 120 - RNA, 21 nt, Homo sapiens
caaagcgcuucccuuuggagc

SEQ ID NO: 121 - RNA, 23 nt, Homo sapiens
ucucuggagggaagcacuuucug

SEQ ID NO: 122 - RNA, 22 nt, Homo sapiens
aaagcgcuucccuucagagugu

SEQ ID NO: 123 - RNA, 21 nt, Homo sapiens
aaagcgcuucucuuuagagga

SEQ ID NO: 124 - RNA, 22 nt, Homo sapiens
acaaagugcuucccuuuagagu

SEQ ID NO: 125 - RNA, 21 nt, Homo sapiens
aacgcgcuucccuauagaggg

SEQ ID NO: 126 - RNA, 22 nt, Homo sapiens
cuacaaagggaagcacuuucuc

SEQ ID NO: 127 - RNA, 21 nt, Homo sapiens
cuccagagggaugcacuuucu

SEQ ID NO: 128 - RNA, 21 nt, Homo sapiens
cucuagagggaagcacuuucu

SEQ ID NO: 129 - RNA, 21 nt, Homo sapiens
cugcaaagggaagcccuuucu

SEQ ID NO: 130 - RNA, 22 nt, Mus musculus
uggaauguaaagaaguauguau

SEQ ID NO: 131 - RNA, 22 nt, Rattus norvegicus
uggaauguaaagaaguguguau

SEQ ID NO: 132 - RNA, 21 nt, Pan paniscus
uggaauguaaagaaguaugua

SEQ ID NO: 133 - RNA, 21 nt, Monodelphis domestica
uggaauguaaagaaguaugua

SEQ ID NO: 134 - RNA, 21 nt, Gallus gallus
uggaauguaaagaaguaugua

SEQ ID NO: 135 - RNA, 21 nt, Gallus gallus
uggaauguuaagaaguaugua

SEQ ID NO: 136 - RNA, 21 nt, Xenopus tropicalis
uggaauguuaagaaguaugua

SEQ ID NO: 137 - RNA, 22 nt, Danio rerio
uggaauguaaagaaguauguau

SEQ ID NO: 138 - RNA, 22 nt, Fugu rubripes
uggaauguaaagaaguauguau

SEQ ID NO: 139 - RNA, 22 nt, Tetraodon nigroviridis
uggaauguaaagaaguauguau

SEQ ID NO: 140 - RNA, 22 nt, Mus musculus
uggaauguaaggaagugugugg

SEQ ID NO: 141 - RNA, 22 nt, Rattus norvegicus
uggaauguaaggaagugugugg

SEQ ID NO: 142 - RNA, 22 nt, Macaca nemestrina
uggaauguaaggaagugugugg

SEQ ID NO: 143 - RNA, 22 nt, Pongo pygmaeus
uggaauguaaggaagugugugg

SEQ ID NO: 144 - RNA, 21 nt, Monodelphis domestica
uggaauguaaggaagugugug

SEQ ID NO: 145 - RNA, 22 nt, Gallus gallus
uggaauguaaggaagugugugg

SEQ ID NO: 146 - RNA, 22 nt, Xenopus tropicalis
uggaauguaaggaagugugugg

SEQ ID NO: 147 - RNA, 22 nt, Danio rerio
uggaauguaaggaagugugugg

SEQ ID NO: 148 - RNA, 23 nt, Mus musculus
uggaagacuugugauuuuguugu

SEQ ID NO: 149 - RNA, 23 nt, Rattus norvegicus
uggaagacuagugauuuuguugu

SEQ ID NO: 150 - RNA, 23 nt, Rattus norvegicus
uggaagacuugugauuuuguugu

SEQ ID NO: 151 - RNA, 21 nt, Lagothrix lagotricha
uggaagacuagugauuuuguu

SEQ ID NO: 152 - RNA, 21 nt, Saguinus labiatus
uggaagacuagugauuuuguu

SEQ ID NO: 153 - RNA, 21 nt, Macaca nemestrina
uggaagacuagugauuuuguu

SEQ ID NO: 154 - RNA, 21 nt, Gorilla gorilla
uggaagacuagugauuuuguu

SEQ ID NO: 155 - RNA, 21 nt, Pan paniscus
uggaagacuagugauuuuguu

SEQ ID NO: 156 - RNA, 21 nt, Pan troglodytes
uggaagacuagugauuuuguu

SEQ ID NO: 157 - RNA, 21 nt, Pongo pygmaeus
uggaagacuagugauuuuguu

SEQ ID NO: 158 - RNA, 22 nt, Monodelphis domestica
uggaagacuagugauuuuguug

SEQ ID NO: 159 - RNA, 22 nt, Gallus gallus
uggaagacuagugauuuuguug

SEQ ID NO: 160 - RNA, 22 nt, Gallus gallus
uggaagacuagugauuuuuguu

SEQ ID NO: 161 - RNA, 22 nt, Xenopus tropicalis
uggaagacuagugauuuuguug

SEQ ID NO: 162 - RNA, 24 nt, Bos taurus
uggaagacuagugauuuuguuguu

SEQ ID NO: 163 - RNA, 21 nt, Sus scrofa
uggaagacuagugauuuuguu

SEQ ID NO: 164 - RNA, 23 nt, Danio rerio
uggaagacuagugauuuuguugu

SEQ ID NO: 165 - RNA, 21 nt, Danio rerio
uggaagacuugugauuuuguu

SEQ ID NO: 166 - RNA, 21 nt, Fugu rubripes
uggaagacuagugauuuuguu

SEQ ID NO: 167 - RNA, 23 nt, Tetraodon nigroviridis
uggaagacuagugauuuuguugu

SEQ ID NO: 168 - RNA, 23 nt, Mus musculus
uacccuguagauccgaauuugug

SEQ ID NO: 169 - RNA, 23 nt, Ateles geoffroyi
uacccuguagauccgaauuugug

SEQ ID NO: 170 - RNA, 23 nt, Saguinus labiatus
uacccuguagauccgaauuugug

SEQ ID NO: 171 - RNA, 22 nt, Macaca nemestrina
uacccuguagaaccgaauuugu

SEQ ID NO: 172 - RNA, 23 nt, Gorilla gorilla
uacccuguagauccgaauuugug

SEQ ID NO: 173 - RNA, 22 nt, Gorilla gorilla
uacccuguagaaccgaauuugu

SEQ ID NO: 174 - RNA, 23 nt, Pan paniscus
uacccuguagauccgaauuugug

SEQ ID NO: 175 - RNA, 22 nt, Pan paniscus
uacccuguagaaccgaauuugu

SEQ ID NO: 176 - RNA, 23 nt, Monodelphis domestica
uacccuguagauccgaauuugug

SEQ ID NO: 177 - RNA, 22 nt, Gallus gallus
uacccuguagaaccgaauuugu

SEQ ID NO: 178 - RNA, 23 nt, Xenopus tropicalis
uacccuguagauccgaauuugug

SEQ ID NO: 179 - RNA, 22 nt, Xenopus tropicalis
uacccuguagaaccgaauuugu

SEQ ID NO: 180 - RNA, 22 nt, Xenopus tropicalis
cacccuguagaaucgaauuugu

SEQ ID NO: 181 - RNA, 23 nt, Bos taurus
uacccuguagauccgaauuugug

SEQ ID NO: 182 - RNA, 23 nt, Bos taurus
uacccuguagaaccgaauuugug

SEQ ID NO: 183 - RNA, 22 nt, Danio rerio
uacccuguagauccgaauuugu

SEQ ID NO: 184 - RNA, 23 nt, Danio rerio
uacccuguagaaccgaauuugug

SEQ ID NO: 185 - RNA, 22 nt, Danio rerio
uacccuguagauccggauuugu

SEQ ID NO: 186 - RNA, 23 nt, Danio rerio
uacccuguagaaccgaaugugug

SEQ ID NO: 187 - RNA, 23 nt, Fugu rubripes
uacccuguagaaccgaauuugug

SEQ ID NO: 188 - RNA, 22 nt, Fugu rubripes
uacccuguagauccggauuugu

SEQ ID NO: 189 - RNA, 25 nt, Fugu rubripes
uacccuguagaaccgaaugugugug

SEQ ID NO: 190 - RNA, 23 nt, Tetraodon nigroviridis
uacccuguagaaccgaauuugug

SEQ ID NO: 191 - RNA, 22 nt, Tetraodon nigroviridis
uacccuguagauccggauuugu

SEQ ID NO: 192 - RNA, 25 nt, Tetraodon nigroviridis
uacccuguagaaccgaaugugugug

SEQ ID NO: 193 - RNA, 23 nt, Mus musculus
uaaggugcaucuagugcagauag

SEQ ID NO: 194 - RNA, 23 nt, Rattus norvegicus
uaaggugcaucuagugcagauag

SEQ ID NO: 195 - RNA, 22 nt, Ateles geoffroyi
uaaggugcaucuagugcagaua

SEQ ID NO: 196 - RNA, 22 nt, Lagothrix lagotricha
uaaggugcaucuagugcagaua

SEQ ID NO: 197 - RNA, 22 nt, Saguinus labiatus
uaaggugcaucuagugcagaua

SEQ ID NO: 198 - RNA, 22 nt, Macaca mulatta
uaaggugcaucuagugcagaua

SEQ ID NO: 199 - RNA, 22 nt, Macaca nemestrina
uaaggugcaucuagugcagaua

SEQ ID NO: 200 - RNA, 22 nt, Gorilla gorilla
uaaggugcaucuagugcagaua

SEQ ID NO: 201 - RNA, 22 nt, Pan paniscus
uaaggugcaucuagugcagaua

SEQ ID NO: 202 - RNA, 22 nt, Pan troglodytes
uaaggugcaucuagugcagaua

SEQ ID NO: 203 - RNA, 22 nt, Pongo pygmaeus
uaaggugcaucuagugcagaua

SEQ ID NO: 204 - RNA, 22 nt, Lemur catta
uaaggugcaucuagugcagaua

SEQ ID NO: 205 - RNA, 22 nt, Monodelphis domestica
uaaggugcaucuagugcagaua

SEQ ID NO: 206 - RNA, 22 nt, Gallus gallus
uaaggugcaucuagugcagaua

SEQ ID NO: 207 - RNA, 22 nt, Xenopus tropicalis
uaaggugcaucuagugcagaua

SEQ ID NO: 208 - RNA, 22 nt, Bos taurus
uaaggugcaucuagugcagaua

SEQ ID NO: 209 - RNA, 22 nt, Sus scrofa
uaaggugcaucuagugcagaua

SEQ ID NO: 210 - RNA, 22 nt, Danio rerio
uaaggugcaucuagugcagaua

SEQ ID NO: 211 - RNA, 22 nt, Danio rerio
uaaggugcaucuuguguaguua

SEQ ID NO: 212 - RNA, 22 nt, Fugu rubripes
uaaggugcaucuagugcagaua

SEQ ID NO: 213 - RNA, 22 nt, Tetraodon nigroviridis
uaaggugcaucuagugcagaua

SEQ ID NO: 214 - RNA, 23 nt, Mus musculus
uaaggugcaucuagugcuguuag

SEQ ID NO: 215 - RNA, 22 nt, Gallus gallus
uaaggugcaucuagugcaguua

SEQ ID NO: 216 - RNA, 23 nt, Xenopus tropicalis
uaaggugcaucuagugcaguuag

SEQ ID NO: 217 - RNA, 22 nt, Bos taurus
uaaggugcaucuagugcaguua

SEQ ID NO: 218 - RNA, 22 nt, Danio rerio
uaaggugcauuuagugcagaua

SEQ ID NO: 219 - RNA, 22 nt, Mus musculus
acugcccuaagugcuccuucug

SEQ ID NO: 220 - RNA, 23 nt, Mus musculus
caaagugcucauagugcagguag

SEQ ID NO: 221 - RNA, 22 nt, Ateles geoffroyi
uaaagugcuuauagugcaggua

SEQ ID NO: 222 - RNA, 22 nt, Lagothrix lagotricha
uaaagugcuuauagugcaggua

SEQ ID NO: 223 - RNA, 22 nt, Saguinus labiatus
uaaagugcuuauagugcaggua

SEQ ID NO: 224 - RNA, 22 nt, Macaca mulatta
uaaagugcuuauagugcaggua

SEQ ID NO: 225 - RNA, 22 nt, Macaca nemestrina
uaaagugcuuauagugcaggua

SEQ ID NO: 226 - RNA, 22 nt, Gorilla gorilla
uaaagugcuuauagugcaggua

SEQ ID NO: 227 - RNA, 22 nt, Pan paniscus
uaaagugcuuauagugcaggua

SEQ ID NO: 228 - RNA, 22 nt, Pan troglodytes
uaaagugcuuauagugcaggua

SEQ ID NO: 229 - RNA, 22 nt, Pongo pygmaeus
uaaagugcuuauagugcaggua

SEQ ID NO: 230 - RNA, 22 nt, Lemur catta
uaaagugcuuauagugcaggua

SEQ ID NO: 231 - RNA, 23 nt, Monodelphis domestica
uaaagugcuuauagugcagguag

SEQ ID NO: 232 - RNA, 23 nt, Gallus gallus
caaagugcucauagugcagguag

SEQ ID NO: 233 - RNA, 23 nt, Xenopus tropicalis
caaagugcucauagugcagguag

SEQ ID NO: 234 - RNA, 22 nt, Bos taurus
caaagugcucacagugcaggua

SEQ ID NO: 235 - RNA, 22 nt, Sus scrofa
uaaagugcuuauagugcaggua

SEQ ID NO: 236 - RNA, 23 nt, Danio rerio
caaagugcucacagugcagguag

SEQ ID NO: 237 - RNA, 23 nt, Fugu rubripes
uaaagugcuuauagugcagguag

SEQ ID NO: 238 - RNA, 23 nt, Tetraodon nigroviridis
uaaagugcuuauagugcagguag

SEQ ID NO: 239 - RNA, 23 nt, Mus musculus
caaagugcuaacagugcagguag

SEQ ID NO: 240 - RNA, 24 nt, Ateles geoffroyi
aaaagugcuuacagugcagguagc

SEQ ID NO: 241 - RNA, 24 nt, Saguinus labiatus
aaaagugcuuacagugcagguagc

SEQ ID NO: 242 - RNA, 24 nt, Macaca mulatta
aaaagugcuuacagugcagguagc

SEQ ID NO: 243 - RNA, 24 nt, Macaca nemestrina
aaaagugcuuacagugcagguagc

SEQ ID NO: 244 - RNA, 24 nt, Gorilla gorilla
aaaagugcuuacagugcagguagc

SEQ ID NO: 245 - RNA, 24 nt, Pan paniscus
aaaagugcuuacagugcagguagc

SEQ ID NO: 246 - RNA, 24 nt, Pan troglodytes
aaaagugcuuacagugcagguagc

SEQ ID NO: 247 - RNA, 24 nt, Pongo pygmaeus
aaaagugcuuacagugcagguagc

SEQ ID NO: 248 - RNA, 22 nt, Gallus gallus
aaaagugcuuacagugcaggua

SEQ ID NO: 249 - RNA, 24 nt, Xenopus tropicalis
aaaagugcuuauagugcagguaga

SEQ ID NO: 250 - RNA, 22 nt, Bos taurus
aaaagugcuuacagugcaggua

SEQ ID NO: 251 - RNA, 24 nt, Sus scrofa
aaaagugcuuacagugcagguagc

SEQ ID NO: 252 - RNA, 21 nt, Mus musculus
uaaagugcugacagugcagau

SEQ ID NO: 253 - RNA, 21 nt, Rattus norvegicus
uaaagugcugacagugcagau

SEQ ID NO: 254 - RNA, 21 nt, Ateles geoffroyi
uaaagugcugacagugcagau

SEQ ID NO: 255 - RNA, 21 nt, Lagothrix lagotricha
uaaagugcugacagugcagau

SEQ ID NO: 256 - RNA, 21 nt, Saguinus labiatus
uaaagugcugacagugcagau

SEQ ID NO: 257 - RNA, 21 nt, Macaca mulatta
uaaagugcugacagugcagau

SEQ ID NO: 258 - RNA, 21 nt, Macaca nemestrina
uaaagugcugacagugcagau

SEQ ID NO: 259 - RNA, 21 nt, Gorilla gorilla
uaaagugcugacagugcagau

SEQ ID NO: 260 - RNA, 21 nt, Pan paniscus
uaaagugcugacagugcagau

SEQ ID NO: 261 - RNA, 21 nt, Pan troglodytes
uaaagugcugacagugcagau

SEQ ID NO: 262 - RNA, 21 nt, Pongo pygmaeus
uaaagugcugacagugcagau

SEQ ID NO: 263 - RNA, 22 nt, Mus musculus
uggcucaguucagcaggaacag

SEQ ID NO: 264 - RNA, 22 nt, Rattus norvegicus
uggcucaguucagcaggaacag

SEQ ID NO: 265 - RNA, 22 nt, Macaca mulatta
uggcucaguucagcaggaacag

SEQ ID NO: 266 - RNA, 22 nt, Macaca nemestrina
uggcucaguucagcaggaacag

SEQ ID NO: 267 - RNA, 22 nt, Gorilla gorilla
uggcucaguucagcaggaacag

SEQ ID NO: 268 - RNA, 22 nt, Pan paniscus
uggcucaguucagcaggaacag

SEQ ID NO: 269 - RNA, 22 nt, Pan troglodytes
uggcucaguucagcaggaacag

SEQ ID NO: 270 - RNA, 22 nt, Pongo pygmaeus
uggcucaguucagcaggaacag

SEQ ID NO: 271 - RNA, 16 nt, Monodelphis domestica
uggcucaguucagcag

SEQ ID NO: 272 - RNA, 22 nt, Gallus gallus
uggcucaguucagcaggaacag

SEQ ID NO: 273 - RNA, 21 nt, Xenopus tropicalis
uggcucaguucagcaggacag

SEQ ID NO: 274 - RNA, 22 nt, Bos taurus
uggcucaguucagcaggaacag

SEQ ID NO: 275 - RNA, 22 nt, Sus scrofa
uggcucaguucagcaggaacag

SEQ ID NO: 276 - RNA, 22 nt, Danio rerio
uggcucaguucagcaggaacag

SEQ ID NO: 277 - RNA, 22 nt, Fugu rubripes
uggcucaguucagcaggaacag

SEQ ID NO: 278 - RNA, 22 nt, Tetraodon nigroviridis
uggcucaguucagcaggaacag

SEQ ID NO: 279 - RNA, 22 nt, Mus musculus
cauugcacuugucucggucuga

SEQ ID NO: 280 - RNA, 22 nt, Rattus norvegicus
cauugcacuugucucggucuga

SEQ ID NO: 281 - RNA, 22 nt, Lagothrix lagotricha
cauugcacuugucucggucuga

SEQ ID NO: 282 - RNA, 22 nt, Macaca mulatta
cauugcacuugucucggucuga

SEQ ID NO: 283 - RNA, 22 nt, Macaca nemestrina
cauugcacuugucucggucuga

SEQ ID NO: 284 - RNA, 22 nt, Gorilla gorilla
cauugcacuugucucggucuga

SEQ ID NO: 285 - RNA, 22 nt, Pan paniscus
cauugcacuugucucggucuga

SEQ ID NO: 286 - RNA, 21 nt, Pan troglodytes
cauugcacuugucucggcuga

SEQ ID NO: 287 - RNA, 22 nt, Pongo pygmaeus
cauugcacuugucucggucuga

SEQ ID NO: 288 - RNA, 22 nt, Monodelphis domestica
cauugcacuugucucggucuga

SEQ ID NO: 289 - RNA, 22 nt, Xenopus tropicalis
cauugcacuugucucggucuga

SEQ ID NO: 290 - RNA, 22 nt, Bos taurus
cauugcacuugucucggucuga

SEQ ID NO: 291 - RNA, 22 nt, Danio rerio
cauugcacuugucucggucuga

SEQ ID NO: 292 - RNA, 22 nt, Fugu rubripes
cauugcacuugucucggucuga

SEQ ID NO: 293 - RNA, 22 nt, Tetraodon nigroviridis
cauugcacuugucucggucuga

SEQ ID NO: 294 - RNA, 22 nt, Mus musculus
uauugcacauuacuaaguugca

SEQ ID NO: 295 - RNA, 22 nt, Rattus norvegicus
uauugcacauuacuaaguugca

SEQ ID NO: 296 - RNA, 21 nt, Saguinus labiatus
uauugcacauuacuaaguugc

SEQ ID NO: 297 - RNA, 21 nt, Macaca mulatta
uauugcacauuacuaaguugc

SEQ ID NO: 298 - RNA, 21 nt, Macaca nemestrina
uauugcacauuacuaaguugc

SEQ ID NO: 299 - RNA, 21 nt, Gorilla gorilla
uauugcacauuacuaaguugc

SEQ ID NO: 300 - RNA, 21 nt, Pan paniscus
uauugcacauuacuaaguugc

SEQ ID NO: 301 - RNA, 21 nt, Pan troglodytes
uauugcacauuacuaaguugc

SEQ ID NO: 302 - RNA, 21 nt, Pongo pygmaeus
uauugcacauuacuaaguugc

SEQ ID NO: 303 - RNA, 21 nt, Monodelphis domestica
uauugcacauuacuaaguugc

SEQ ID NO: 304 - RNA, 21 nt, Gallus gallus
uauugcacauuacuaaguugc

SEQ ID NO: 305 - RNA, 21 nt, Sus scrofa
uauugcacauuacuaaguugc

SEQ ID NO: 306 - RNA, 22 nt, Mus musculus
uucaaguaauccaggauaggcu

SEQ ID NO: 307 - RNA, 22 nt, Rattus norvegicus
uucaaguaauccaggauaggcu

SEQ ID NO: 308 - RNA, 22 nt, Lagothrix lagotricha
uucaaguaauccaggauaggcu

SEQ ID NO: 309 - RNA, 22 nt, Macaca mulatta
uucaaguaauccaggauaggcu

SEQ ID NO: 310 - RNA, 22 nt, Macaca nemestrina
uucaaguaauccaggauaggcu

SEQ ID NO: 311 - RNA, 22 nt, Gorilla gorilla
uucaaguaauccaggauaggcu

SEQ ID NO: 312 - RNA, 22 nt, Pan paniscus
uucaaguaauccaggauaggcu

SEQ ID NO: 313 - RNA, 22 nt, Pan troglodytes
uucaaguaauccaggauaggcu

SEQ ID NO: 314 - RNA, 22 nt, Pongo pygmaeus
uucaaguaauccaggauaggcu

SEQ ID NO: 315 - RNA, 21 nt, Gallus gallus
uucaaguaauccaggauaggc

SEQ ID NO: 316 - RNA, 22 nt, Bos taurus
uucaaguaauccaggauaggcu

SEQ ID NO: 317 - RNA, 22 nt, Sus scrofa
uucaaguaauccaggauaggcu

SEQ ID NO: 318 - RNA, 22 nt, Danio rerio
uucaaguaauccaggauaggcu

SEQ ID NO: 319 - RNA, 22 nt, Fugu rubripes
uucaaguaauccaggauaggcu

SEQ ID NO: 320 - RNA, 22 nt, Tetraodon nigroviridis
uucaaguaauccaggauaggcu

SEQ ID NO: 321 - RNA, 21 nt, Mus musculus
uucaaguaauucaggauaggu

SEQ ID NO: 322 - RNA, 21 nt, Rattus norvegicus
uucaaguaauucaggauaggu

SEQ ID NO: 323 - RNA, 22 nt, Bos taurus
uucaaguaauucaggauagguu

SEQ ID NO: 324 - RNA, 22 nt, Danio rerio
uucaaguaauccaggauagguu

SEQ ID NO: 325 - RNA, 21 nt, Mus musculus
uucacaguggcuaaguucugc

SEQ ID NO: 326 - RNA, 21 nt, Rattus norvegicus
uucacaguggcuaaguuccgc

SEQ ID NO: 327 - RNA, 21 nt, Rattus norvegicus
uucacaguggcuaaguucugc

SEQ ID NO: 328 - RNA, 22 nt, Ateles geoffroyi
uucacaguggcuaaguuccgcc

SEQ ID NO: 329 - RNA, 22 nt, Saguinus labiatus
uucacaguggcuaaguuccgcc

SEQ ID NO: 330 - RNA, 22 nt, Macaca mulatta
uucacaguggcuaaguuccgcc

SEQ ID NO: 331 - RNA, 22 nt, Macaca nemestrina
uucacaguggcuaaguuccgcc

SEQ ID NO: 332 - RNA, 22 nt, Gorilla gorilla
uucacaguggcuaaguuccgcc

SEQ ID NO: 333 - RNA, 22 nt, Pan paniscus
uucacaguggcuaaguuccgcc

SEQ ID NO: 334 - RNA, 22 nt, Pan troglodytes
uucacaguggcuaaguuccgcc

SEQ ID NO: 335 - RNA, 22 nt, Pongo pygmaeus
uucacaguggcuaaguuccgcc

SEQ ID NO: 336 - RNA, 22 nt, Lemur catta
uucacaguggcuaaguuccgcc

SEQ ID NO: 337 - RNA, 21 nt, Monodelphis domestica
uucacaguggcuaaguuccgc

SEQ ID NO: 338 - RNA, 21 nt, Monodelphis domestica
uucacaguggcuaaguucugc

SEQ ID NO: 339 - RNA, 21 nt, Gallus gallus
uucacaguggcuaaguucugc

SEQ ID NO: 340 - RNA, 21 nt, Xenopus tropicalis
uucacaguggcuaaguuccgc

SEQ ID NO: 341 - RNA, 21 nt, Xenopus tropicalis
uucacaguggcuaaguucugc

SEQ ID NO: 342 - RNA, 21 nt, Xenopus tropicalis
uucacaguggcuaaguuccac

SEQ ID NO: 343 - RNA, 20 nt, Bos taurus
uucacaguggcuaaguuccg

SEQ ID NO: 344 - RNA, 21 nt, Bos taurus
uucacaguggcuaaguucugc

SEQ ID NO: 345 - RNA, 21 nt, Sus scrofa
uucacaguggcuaaguuccgc

SEQ ID NO: 346 - RNA, 22 nt, Danio rerio
uucacaguggcuaaguuccgcu

SEQ ID NO: 347 - RNA, 22 nt, Danio rerio
uucacaguggcuaaguucugca

SEQ ID NO: 348 - RNA, 21 nt, Danio rerio
uucacagugguuaaguucugc

SEQ ID NO: 349 - RNA, 22 nt, Danio rerio
uucacaguggcuaaguucuuca

SEQ ID NO: 350 - RNA, 22 nt, Danio rerio
uucacaguggcuaaguucagug

SEQ ID NO: 351 - RNA, 22 nt, Fugu rubripes
uucacaguggcuaaguucugca

SEQ ID NO: 352 - RNA, 21 nt, Fugu rubripes
uucacagugguuaaguucugc

SEQ ID NO: 353 - RNA, 22 nt, Fugu rubripes
uucacaguggcuaaguucagug

SEQ ID NO: 354 - RNA, 22 nt, Tetraodon nigroviridis
uucacaguggcuaaguucugca

SEQ ID NO: 355 - RNA, 21 nt, Tetraodon nigroviridis
uucacagugguuaaguucugc

SEQ ID NO: 356 - RNA, 22 nt, Tetraodon nigroviridis
uucacaguggcuaaguucagug

SEQ ID NO: 357 - RNA, 22 nt, Mus musculus
aaggagcucacagucuauugag

SEQ ID NO: 358 - RNA, 22 nt, Rattus norvegicus
aaggagcucacagucuauugag

SEQ ID NO: 359 - RNA, 22 nt, Ateles geoffroyi
aaggagcucacagucuauugag

SEQ ID NO: 360 - RNA, 22 nt, Lagothrix lagotricha
aaggagcucacagucuauugag

SEQ ID NO: 361 - RNA, 22 nt, Saguinus labiatus
aaggagcucacagucuauugag

SEQ ID NO: 362 - RNA, 22 nt, Macaca mulatta
aaggagcucacagucuauugag

SEQ ID NO: 363 - RNA, 22 nt, Macaca nemestrina
aaggagcucacagucuauugag

SEQ ID NO: 364 - RNA, 22 nt, Gorilla gorilla
aaggagcucacagucuauugag

SEQ ID NO: 365 - RNA, 22 nt, Pan paniscus
aaggagcucacagucuauugag

SEQ ID NO: 366 - RNA, 22 nt, Pan troglodytes
aaggagcucacagucuauugag

SEQ ID NO: 367 - RNA, 22 nt, Pongo pygmaeus
aaggagcucacagucuauugag

SEQ ID NO: 368 - RNA, 22 nt, Sus scrofa
aaggagcucacacucuauugag

SEQ ID NO: 369 - RNA, 22 nt, Mus musculus
uagcaccaucugaaaucgguua

SEQ ID NO: 370 - RNA, 22 nt, Rattus norvegicus
uagcaccaucugaaaucgguua

SEQ ID NO: 371 - RNA, 23 nt, Rattus norvegicus
uagcaccauuugaaaucaguguu

SEQ ID NO: 372 - RNA, 20 nt, Ateles geoffroyi
uagcaccauuugaaaucagu

SEQ ID NO: 373 - RNA, 20 nt, Lagothrix lagotricha
uagcaccauuugaaaucagu

SEQ ID NO: 374 - RNA, 20 nt, Saguinus labiatus
uagcaccauuugaaaucagu

SEQ ID NO: 375 - RNA, 20 nt, Macaca nemestrina
uagcaccauuugaaaucagu

SEQ ID NO: 376 - RNA, 20 nt, Gorilla gorilla
uagcaccauuugaaaucagu

SEQ ID NO: 377 - RNA, 20 nt, Pan paniscus
uagcaccauuugaaaucagu

SEQ ID NO: 378 - RNA, 20 nt, Pan troglodytes
uagcaccauuugaaaucagu

SEQ ID NO: 379 - RNA, 20 nt, Pongo pygmaeus
uagcaccauuugaaaucagu

SEQ ID NO: 380 - RNA, 20 nt, Monodelphis domestica
uagcaccauuugaaaucggu

SEQ ID NO: 381 - RNA, 20 nt, Monodelphis domestica
uagcaccauuugaaaucagu

SEQ ID NO: 382 - RNA, 21 nt, Gallus gallus
uagcaccauuugaaaucgguu

SEQ ID NO: 383 - RNA, 23 nt, Gallus gallus
uagcaccauuugaaaucaguguu

SEQ ID NO: 384 - RNA, 21 nt, Xenopus tropicalis
uagcaccauuugaaaucgguu

SEQ ID NO: 385 - RNA, 23 nt, Xenopus tropicalis
uagcaccauuugaaaucaguguu

SEQ ID NO: 386 - RNA, 23 nt, Xenopus tropicalis
uagcaccauaugaaaucaguguc

SEQ ID NO: 387 - RNA, 24 nt, Bos taurus
uagcaccauuugaaaucaguguuu

SEQ ID NO: 388 - RNA, 20 nt, Sus scrofa
uagcaccauuugaaaucagu

SEQ ID NO: 389 - RNA, 22 nt, Danio rerio
uagcaccauuugaaaucgguua

SEQ ID NO: 390 - RNA, 22 nt, Danio rerio
uagcaccauuugaaaucagugu

SEQ ID NO: 391 - RNA, 22 nt, Fugu rubripes
uagcaccauuugaaaucgguua

SEQ ID NO: 392 - RNA, 22 nt, Fugu rubripes
uagcaccauuugaaaucagugu

SEQ ID NO: 393 - RNA, 22 nt, Tetraodon nigroviridis
uagcaccauuugaaaucgguua

SEQ ID NO: 394 - RNA, 22 nt, Tetraodon nigroviridis
uagcaccauuugaaaucagugu

SEQ ID NO: 395 - RNA, 22 nt, Mus musculus
uagcaccauuugaaaucgguua

SEQ ID NO: 396 - RNA, 22 nt, Rattus norvegicus
uagcaccauuugaaaucgguua

SEQ ID NO: 397 - RNA, 20 nt, Gallus gallus
uagcaccauuugaaaucggu

SEQ ID NO: 398 - RNA, 20 nt, Xenopus tropicalis
uagcaccauuugaaaucggu

SEQ ID NO: 399 - RNA, 22 nt, Bos taurus
uagcaccauuugaaaucgguua

SEQ ID NO: 400 - RNA, 22 nt, Sus scrofa
uagcaccauuugaaaucgguua

SEQ ID NO: 401 - RNA, 22 nt, Mus musculus
uguaaacauccuacacucagcu

SEQ ID NO: 402 - RNA, 22 nt, Mus musculus
uguaaacauccccgacuggaag

SEQ ID NO: 403 - RNA, 22 nt, Mus musculus
uguaaacauccuugacuggaag

SEQ ID NO: 404 - RNA, 22 nt, Rattus norvegicus
uguaaacauccucgacuggaag

SEQ ID NO: 405 - RNA, 22 nt, Rattus norvegicus
uguaaacauccccgacuggaag

SEQ ID NO: 406 - RNA, 22 nt, Rattus norvegicus
uguaaacauccuugacuggaag

SEQ ID NO: 407 - RNA, 21 nt, Ateles geoffroyi
uguaaacauccuacacucagc

SEQ ID NO: 408 - RNA, 21 nt, Lagothrix lagotricha
uguaaacauccuacacucagc

SEQ ID NO: 409 - RNA, 22 nt, Macaca mulatta
uguaaacauccucgacuggaag

SEQ ID NO: 410 - RNA, 21 nt, Macaca mulatta
uguaaacauccuacacucagc

SEQ ID NO: 411 - RNA, 21 nt, Macaca nemestrina
uguaaacauccuacacucagc

SEQ ID NO: 412 - RNA, 22 nt, Macaca nemestrina
uguaaacauccccgacuggaag

SEQ ID NO: 413 - RNA, 22 nt, Gorilla gorilla
uguaaacauccucgacuggaag

SEQ ID NO: 414 - RNA, 21 nt, Gorilla gorilla
uguaaacauccuacacucagc

SEQ ID NO: 415 - RNA, 22 nt, Gorilla gorilla
uguaaacauccccgacuggaag

SEQ ID NO: 416 - RNA, 22 nt, Pan paniscus
uguaaacauccucgacuggaag

SEQ ID NO: 417 - RNA, 21 nt, Pan paniscus
uguaaacauccuacacucagc

SEQ ID NO: 418 - RNA, 22 nt, Pan paniscus
uguaaacauccccgacuggaag

SEQ ID NO: 419 - RNA, 22 nt, Pan troglodytes
uguaaacauccucgacuggaag

SEQ ID NO: 420 - RNA, 21 nt, Pan troglodytes
uguaaacauccuacacucagc

SEQ ID NO: 421 - RNA, 22 nt, Pan troglodytes
uguaaacauccccgacuggaag

SEQ ID NO: 422 - RNA, 22 nt, Pongo pygmaeus
uguaaacauccucgacuggaag

SEQ ID NO: 423 - RNA, 22 nt, Monodelphis domestica
uguaaacauccucgacuggaag

SEQ ID NO: 424 - RNA, 22 nt, Gallus gallus
uguaaacauccucgacuggaag

SEQ ID NO: 425 - RNA, 22 nt, Gallus gallus
uguaaacauccuacacucagcu

SEQ ID NO: 426 - RNA, 22 nt, Gallus gallus
uguaaacauccccgacuggaag

SEQ ID NO: 427 - RNA, 19 nt, Gallus gallus
uguaaacauccuugacugg

SEQ ID NO: 428 - RNA, 22 nt, Xenopus tropicalis
uguaaacauccucgacuggaag

SEQ ID NO: 429 - RNA, 22 nt, Xenopus tropicalis
uguaaacauccuacacucagcu

SEQ ID NO: 430 - RNA, 22 nt, Xenopus tropicalis
uguaaacauccccgacuggaag

SEQ ID NO: 431 - RNA, 20 nt, Xenopus tropicalis
uguaaacauccuugacugga

SEQ ID NO: 432 - RNA, 24 nt, Bos taurus
uguaaacauccucgacuggaagcu

SEQ ID NO: 433 - RNA, 22 nt, Bos taurus
uguaaacauccuacacucagcu

SEQ ID NO: 434 - RNA, 24 nt, Bos taurus
uguaaacauccccgacuggaagcu

SEQ ID NO: 435 - RNA, 24 nt, Bos taurus
uguaaacauccuugacuggaagcu

SEQ ID NO: 436 - RNA, 22 nt, Danio rerio
uguaaacauucccgacuggaag

SEQ ID NO: 437 - RNA, 22 nt, Danio rerio
uguaaacauccuacacucagcu

SEQ ID NO: 438 - RNA, 22 nt, Danio rerio
uguaaacauccccgacuggaag

SEQ ID NO: 439 - RNA, 22 nt, Danio rerio
uguaaacauccuugacuggaag

SEQ ID NO: 440 - RNA, 22 nt, Fugu rubripes
uguaaacauccuacacucagcu

SEQ ID NO: 441 - RNA, 22 nt, Fugu rubripes
uguaaacauccccgacuggaag

SEQ ID NO: 442 - RNA, 22 nt, Tetraodon nigroviridis
uguaaacauccuacacucagcu

SEQ ID NO: 443 - RNA, 22 nt, Tetraodon nigroviridis
uguaaacauccccgacuggaag

SEQ ID NO: 444 - RNA, 23 nt, Mus musculus
uguaaacauccuacacucucagc

SEQ ID NO: 445 - RNA, 23 nt, Rattus norvegicus
uguaaacauccuacacucucagc

SEQ ID NO: 446 - RNA, 23 nt, Lagothrix lagotricha
uguaaacauccuacacucucagc

SEQ ID NO: 447 - RNA, 23 nt, Macaca nemestrina
uguaaacauccuacacucucagc

SEQ ID NO: 448 - RNA, 23 nt, Pan troglodytes
uguaaacauccuacacucucagc

SEQ ID NO: 449 - RNA, 24 nt, Gallus gallus
uguaaacauccuacacucucagcu

SEQ ID NO: 450 - RNA, 23 nt, Xenopus tropicalis
uguaaacauccuacacucucagc

SEQ ID NO: 451 - RNA, 23 nt, Bos taurus
uguaaacauccuacacucucagc

SEQ ID NO: 452 - RNA, 23 nt, Sus scrofa
uguaaacauccuacacucucagc

SEQ ID NO: 453 - RNA, 22 nt, Danio rerio
uguaaacauccuacacucucag

SEQ ID NO: 454 - RNA, 22 nt, Fugu rubripes
uguaaacauccuacacucucgg

SEQ ID NO: 455 - RNA, 22 nt, Tetraodon nigroviridis
uguaaacauccuacacucucgg

SEQ ID NO: 456 - RNA, 22 nt, Ateles geoffroyi
aaagugcuguucgugcagguag

SEQ ID NO: 457 - RNA, 22 nt, Lagothrix lagotricha
aaagugcuguucgugcagguag

SEQ ID NO: 458 - RNA, 22 nt, Saguinus labiatus
aaagugcuguucgugcagguag

SEQ ID NO: 459 - RNA, 22 nt, Macaca mulatta
aaagugcuguucgugcagguag

SEQ ID NO: 460 - RNA, 22 nt, Macaca nemestrina
aaagugcuguucgugcagguag

SEQ ID NO: 461 - RNA, 22 nt, Gorilla gorilla
aaagugcuguucgugcagguag

SEQ ID NO: 462 - RNA, 22 nt, Pan paniscus
aaagugcuguucgugcagguag

SEQ ID NO: 463 - RNA, 22 nt, Pan troglodytes
aaagugcuguucgugcagguag

SEQ ID NO: 464 - RNA, 22 nt, Pongo pygmaeus
aaagugcuguucgugcagguag

SEQ ID NO: 465 - RNA, 22 nt, Monodelphis domestica
aaagugcuguucgugcagguag

SEQ ID NO: 466 - RNA, 23 nt, Mus musculus
uaagugcuuccauguuuuaguag

SEQ ID NO: 467 - RNA, 23 nt, Mus musculus
uaagugcuuccauguuugagugu

SEQ ID NO: 468 - RNA, 20 nt, Monodelphis domestica
uaagugcuuccauguuuuag

SEQ ID NO: 469 - RNA, 20 nt, Monodelphis domestica
uaagugcuuccauguuuugg

SEQ ID NO: 470 - RNA, 21 nt, Monodelphis domestica
uaagugcuuccauguuucagu

SEQ ID NO: 471 - RNA, 21 nt, Monodelphis domestica
uaagugcuuccauguuugagu

SEQ ID NO: 472 - RNA, 23 nt, Gallus gallus
uaagugcuuccauguuuuaguag

SEQ ID NO: 473 - RNA, 23 nt, Gallus gallus
uaagugcuuccauguuucagugg

SEQ ID NO: 474 - RNA, 23 nt, Gallus gallus
uaagugcuuccauguuuuaguug

SEQ ID NO: 475 - RNA, 23 nt, Xenopus tropicalis
uaagugcuccaauguuuuagugg

SEQ ID NO: 476 - RNA, 22 nt, Lagothrix lagotricha
uucaacggguauuuauugagca

SEQ ID NO: 477 - RNA, 22 nt, Saguinus labiatus
uucaacggguauuuauugagca

SEQ ID NO: 478 - RNA, 22 nt, Gorilla gorilla
uucaacggguauuuauugagca

SEQ ID NO: 479 - RNA, 22 nt, Pan paniscus
uucaacggguauuuauugagca

SEQ ID NO: 480 - RNA, 22 nt, Pan troglodytes
uucaacggguauuuauugagca

SEQ ID NO: 481 - RNA, 22 nt, Pongo pygmaeus
uucaacggguauuuauugagca

SEQ ID NO: 482 - RNA, 22 nt, Sus scrofa
uucaacggguauuuauugagca

SEQ ID NO: 483 - RNA, 22 nt, Mus musculus
ugagguaguaaguuguauuguu

SEQ ID NO: 484 - RNA, 22 nt, Rattus norvegicus
ugagguaguaaguuguauuguu

SEQ ID NO: 485 - RNA, 22 nt, Ateles geoffroyi
ugagguaguaaguuguauuguu

SEQ ID NO: 486 - RNA, 22 nt, Macaca mulatta
ugagguaguaaguuguauuguu

SEQ ID NO: 487 - RNA, 22 nt, Gorilla gorilla
ugagguaguaaguuguauuguu

SEQ ID NO: 488 - RNA, 22 nt, Pan paniscus
ugagguaguaaguuguauuguu

SEQ ID NO: 489 - RNA, 22 nt, Pan troglodytes
ugagguaguaaguuguauuguu

SEQ ID NO: 490 - RNA, 22 nt, Pongo pygmaeus
ugagguaguaaguuguauuguu

SEQ ID NO: 491 - RNA, 22 nt, Xenopus tropicalis
ugagguaguaaguuguauuguu

SEQ ID NO: 492 - RNA, 22 nt, Bos taurus
ugagguaguaaguuguauuguu

SEQ ID NO: 493 - RNA, 22 nt, Mus musculus
cagugcaauguuaaaagggcau

SEQ ID NO: 494 - RNA, 22 nt, Rattus norvegicus
cagugcaauguuaaaagggcau

SEQ ID NO: 495 - RNA, 20 nt, Macaca mulatta
cagugcaauguuaaaagggc

SEQ ID NO: 496 - RNA, 20 nt, Macaca nemestrina
cagugcaauguuaaaagggc

SEQ ID NO: 497 - RNA, 20 nt, Gorilla gorilla
cagugcaauguuaaaagggc

SEQ ID NO: 498 - RNA, 20 nt, Pan paniscus
cagugcaauguuaaaagggc

SEQ ID NO: 499 - RNA, 22 nt, Monodelphis domestica
cagugcaauguaaaaagggcau

SEQ ID NO: 500 - RNA, 22 nt, Gallus gallus
cagugcaauauuaaaagggcau

SEQ ID NO: 501 - RNA, 22 nt, Xenopus tropicalis
cagugcaauguuaaaagggcau

SEQ ID NO: 502 - RNA, 22 nt, Danio rerio
cagugcaauguuaaaagggcau

SEQ ID NO: 503 - RNA, 22 nt, Danio rerio
cagugcaauauuaaaagggcau

SEQ ID NO: 504 - RNA, 22 nt, Fugu rubripes
cagugcaauauuaaaagggcau

SEQ ID NO: 505 - RNA, 22 nt, Tetraodon nigroviridis
cagugcaauauuaaaagggcau

SEQ ID NO: 506 - RNA, 22 nt, Mus musculus
cagugcaaugaugaaagggcau

SEQ ID NO: 507 - RNA, 22 nt, Rattus norvegicus
cagugcaaugaugaaagggcau

SEQ ID NO: 508 - RNA, 22 nt, Gallus gallus
cagugcaauaaugaaagggcgu

SEQ ID NO: 509 - RNA, 22 nt, Xenopus tropicalis
cagugcaaugaugaaagggcau

SEQ ID NO: 510 - RNA, 22 nt, Danio rerio
cagugcaauaaugaaagggcau

SEQ ID NO: 511 - RNA, 23 nt, Mus musculus
cagugcaauaguauugucaaagc

SEQ ID NO: 512 - RNA, 23 nt, Mus musculus
cagugcaaugguauugucaaagc

SEQ ID NO: 513 - RNA, 23 nt, Rattus norvegicus
cagugcaauaguauugucaaagc

SEQ ID NO: 514 - RNA, 23 nt, Rattus norvegicus
cagugcaaugguauugucaaagc

SEQ ID NO: 515 - RNA, 25 nt, Gallus gallus
cagugcaauaauauugucaaagcau

SEQ ID NO: 516 - RNA, 23 nt, Xenopus tropicalis
cagugcaauaguauugucaaagc

SEQ ID NO: 517 - RNA, 22 nt, Danio rerio
cagugcaauaguauugucaaag

SEQ ID NO: 518 - RNA, 22 nt, Danio rerio
cagugcaauaguauugucauug

SEQ ID NO: 519 - RNA, 22 nt, Danio rerio
cagugcaauaguauugucauag

SEQ ID NO: 520 - RNA, 22 nt, Fugu rubripes
cagugcaauaguauugucauag

SEQ ID NO: 521 - RNA, 22 nt, Tetraodon nigroviridis
cagugcaauaguauugucauag

SEQ ID NO: 522 - RNA, 22 nt, Mus musculus
uaacagucuacagccauggucg

SEQ ID NO: 523 - RNA, 22 nt, Rattus norvegicus
uaacagucuacagccauggucg

SEQ ID NO: 524 - RNA, 22 nt, Monodelphis domestica
uaacagucuacagccauggucg

SEQ ID NO: 525 - RNA, 22 nt, Xenopus tropicalis
uaacagucuacagccauggucg

SEQ ID NO: 526 - RNA, 22 nt, Bos taurus
uaacagucuacagccauggucg

SEQ ID NO: 527 - RNA, 22 nt, Danio rerio
uaacagucuacagccauggucg

SEQ ID NO: 528 - RNA, 22 nt, Fugu rubripes
uaacagucuacagccauggucg

SEQ ID NO: 529 - RNA, 22 nt, Tetraodon nigroviridis
uaacagucuacagccauggucg

SEQ ID NO: 530 - RNA, 22 nt, Mus musculus
uaacagucuccagucacggcca

SEQ ID NO: 531 - RNA, 22 nt, Rattus norvegicus
uaacagucuccagucacggcca

SEQ ID NO: 532 - RNA, 21 nt, Monodelphis domestica
uaacagucuccagucacggcc

SEQ ID NO: 533 - RNA, 21 nt, Xenopus tropicalis
uaacagucuacagucauggcu

SEQ ID NO: 534 - RNA, 21 nt, Danio rerio
uaacagucuacagucauggcu

SEQ ID NO: 535 - RNA, 21 nt, Fugu rubripes
uaacagucuacagucauggcu

SEQ ID NO: 536 - RNA, 21 nt, Tetraodon nigroviridis
uaacagucuacagucauggcu

SEQ ID NO: 537 - RNA, 22 nt, Ateles geoffroyi
uugguccccuucaaccagcugu

SEQ ID NO: 538 - RNA, 22 nt, Lagothrix lagotricha
uugguccccuucaaccagcugu

SEQ ID NO: 539 - RNA, 22 nt, Saguinus labiatus
uugguccccuucaaccagcugu

SEQ ID NO: 540 - RNA, 22 nt, Macaca nemestrina
uugguccccuucaaccagcugu

SEQ ID NO: 541 - RNA, 22 nt, Gorilla gorilla
uugguccccuucaaccagcugu

SEQ ID NO: 542 - RNA, 22 nt, Pan paniscus
uugguccccuucaaccagcugu

SEQ ID NO: 543 - RNA, 21 nt, Pan troglodytes
uugguccccuucaaccagcug

SEQ ID NO: 544 - RNA, 22 nt, Monodelphis domestica
uugguccccuucaaccagcugu

SEQ ID NO: 545 - RNA, 22 nt, Gallus gallus
uugguccccuucaaccagcugu

SEQ ID NO: 546 - RNA, 22 nt, Gallus gallus
uugguccccuucaaccagcugc

SEQ ID NO: 547 - RNA, 22 nt, Xenopus laevis
uugguccccuucaaccagcugu

SEQ ID NO: 548 - RNA, 22 nt, Xenopus tropicalis
uugguccccuucaaccagcugu

SEQ ID NO: 549 - RNA, 22 nt, Xenopus tropicalis
uugguccccuucaaccagccgc

SEQ ID NO: 550 - RNA, 21 nt, Gallus gallus
uugguccccuucaaccagcua

SEQ ID NO: 551 - RNA, 21 nt, Xenopus tropicalis
uugguccccuucaaccagcua

SEQ ID NO: 552 - RNA, 22 nt, Mus musculus
ugugacugguugaccagagggg

SEQ ID NO: 553 - RNA, 22 nt, Rattus norvegicus
ugugacugguugaccagagggg

SEQ ID NO: 554 - RNA, 21 nt, Macaca nemestrina
ugugacugguugaccagaggg

SEQ ID NO: 555 - RNA, 21 nt, Gorilla gorilla
ugugacugguugaccagaggg

SEQ ID NO: 556 - RNA, 21 nt, Pan paniscus
ugugacugguugaccagaggg

SEQ ID NO: 557 - RNA, 21 nt, Pongo pygmaeus
ugugacugguugaccagaggg

SEQ ID NO: 558 - RNA, 22 nt, Monodelphis domestica
uauugcuuaagaauacgcguag

SEQ ID NO: 559 - RNA, 22 nt, Gallus gallus
uauugcuuaagaauacgcguag

SEQ ID NO: 560 - RNA, 22 nt, Xenopus tropicalis
uauugcuuaagaauacgcguag

SEQ ID NO: 561 - RNA, 23 nt, Mus musculus
uguaguguuuccuacuuuaugga

SEQ ID NO: 562 - RNA, 23 nt, Rattus norvegicus
uguaguguuuccuacuuuaugga

SEQ ID NO: 563 - RNA, 22 nt, Gallus gallus
uguaguguuuccuacuuuaugg

SEQ ID NO: 564 - RNA, 23 nt, Xenopus tropicalis
uguaguguuuccuacuuuaugga

SEQ ID NO: 565 - RNA, 22 nt, Mus musculus
ugagaacugaauuccauggguu

SEQ ID NO: 566 - RNA, 22 nt, Rattus norvegicus
ugagaacugaauuccauggguu

SEQ ID NO: 567 - RNA, 22 nt, Gallus gallus
ugagaacugaauuccauggguu

SEQ ID NO: 568 - RNA, 22 nt, Gallus gallus
ugagaacugaauuccauaggcg

SEQ ID NO: 569 - RNA, 22 nt, Xenopus tropicalis
ugagaacugaauuccauagguu

SEQ ID NO: 570 - RNA, 23 nt, Danio rerio
ugagaacugaauuccauagaugg

SEQ ID NO: 571 - RNA, 22 nt, Danio rerio
ugagaacugaauuccaagggug

SEQ ID NO: 572 - RNA, 20 nt, Saguinus labiatus
guguguggaaaugcuucugc

SEQ ID NO: 573 - RNA, 20 nt, Macaca nemestrina
guguguggaaaugcuucugc

SEQ ID NO: 574 - RNA, 20 nt, Pan paniscus
guguguggaaaugcuucugc

SEQ ID NO: 575 - RNA, 20 nt, Pan troglodytes
guguguggaaaugcuucugc

SEQ ID NO: 576 - RNA, 20 nt, Pongo pygmaeus
guguguggaaaugcuucugc

SEQ ID NO: 577 - RNA, 23 nt, Mus musculus
ucuggcuccgugucuucacuccc

SEQ ID NO: 578 - RNA, 22 nt, Mus musculus
aacauucaaccugucggugagu

SEQ ID NO: 579 - RNA, 22 nt, Rattus norvegicus
aacauucaaccugucggugagu

SEQ ID NO: 580 - RNA, 22 nt, Macaca mulatta
aacauucaaccugucggugagu

SEQ ID NO: 581 - RNA, 22 nt, Gorilla gorilla
aacauucaaccugucggugagu

SEQ ID NO: 582 - RNA, 22 nt, Pan paniscus
aacauucaaccugucggugagu

SEQ ID NO: 583 - RNA, 22 nt, Pan troglodytes
aacauucaaccugucggugagu

SEQ ID NO: 584 - RNA, 23 nt, Monodelphis domestica
aacauucaacgcugucggugagu

SEQ ID NO: 585 - RNA, 24 nt, Bos taurus
aacauucaaccugucggugaguuu

SEQ ID NO: 586 - RNA, 22 nt, Sus scrofa
aacauucaaccugucggugagu

SEQ ID NO: 587 - RNA, 22 nt, Danio rerio
cacauucauugcugucgguggg

SEQ ID NO: 588 - RNA, 23 nt, Mus musculus
aacauucauuguugucggugggu

SEQ ID NO: 589 - RNA, 23 nt, Rattus norvegicus
aacauucauuguugucggugggu

SEQ ID NO: 590 - RNA, 21 nt, Xenopus tropicalis
ugguucuagacuugccaacua

SEQ ID NO: 591 - RNA, 21 nt, Danio rerio
ugguucuagacuugccaacua

SEQ ID NO: 592 - RNA, 22 nt, Mus musculus
uauggcacugguagaauucacu

SEQ ID NO: 593 - RNA, 22 nt, Rattus norvegicus
uauggcacugguagaauucacu

SEQ ID NO: 594 - RNA, 23 nt, Saguinus labiatus
uauggcacugguagaauucacug

SEQ ID NO: 595 - RNA, 23 nt, Macaca mulatta
uauggcacugguagaauucacug

SEQ ID NO: 596 - RNA, 23 nt, Macaca nemestrina
uauggcacugguagaauucacug

SEQ ID NO: 597 - RNA, 23 nt, Gorilla gorilla
uauggcacugguagaauucacug

SEQ ID NO: 598 - RNA, 23 nt, Pan paniscus
uauggcacugguagaauucacug

SEQ ID NO: 599 - RNA, 23 nt, Pan troglodytes
uauggcacugguagaauucacug

SEQ ID NO: 600 - RNA, 23 nt, Monodelphis domestica
uauggcacugguagaauucacug

SEQ ID NO: 601 - RNA, 23 nt, Gallus gallus
uauggcacugguagaauucacug

SEQ ID NO: 602 - RNA, 23 nt, Xenopus tropicalis
uauggcacugguagaauucacug

SEQ ID NO: 603 - RNA, 23 nt, Sus scrofa
uauggcacugguagaauucacug

SEQ ID NO: 604 - RNA, 23 nt, Danio rerio
uauggcacugguagaauucacug

SEQ ID NO: 605 - RNA, 23 nt, Fugu rubripes
uauggcacugguagaauucacug

SEQ ID NO: 606 - RNA, 23 nt, Tetraodon nigroviridis
uauggcacugguagaauucacug

SEQ ID NO: 607 - RNA, 22 nt, Mus musculus
uggacggagaacugauaagggu

SEQ ID NO: 608 - RNA, 22 nt, Rattus norvegicus
uggacggagaacugauaagggu

SEQ ID NO: 609 - RNA, 22 nt, Macaca nemestrina
uggacggagaacugauaagggu

SEQ ID NO: 610 - RNA, 22 nt, Pan troglodytes
uggacggagaacugauaagggu

SEQ ID NO: 611 - RNA, 22 nt, Pongo pygmaeus
uggacggagaacugauaagggu

SEQ ID NO: 612 - RNA, 22 nt, Monodelphis domestica
uggacggagaacugauaagggu

SEQ ID NO: 613 - RNA, 22 nt, Gallus gallus
uggacggagaacugauaagggu

SEQ ID NO: 614 - RNA, 22 nt, Xenopus tropicalis
uggacggagaacugauaaggcu

SEQ ID NO: 615 - RNA, 22 nt, Sus scrofa
uggacggagaacugauaagggu

SEQ ID NO: 616 - RNA, 22 nt, Danio rerio
uggacggagaacugauaagggc

SEQ ID NO: 617 - RNA, 21 nt, Fugu rubripes
uggacggagaacugauaaggg

SEQ ID NO: 618 - RNA, 21 nt, Tetraodon nigroviridis
uggacggagaacugauaaggg

SEQ ID NO: 619 - RNA, 21 nt, Mus musculus
cugaccuaugaauugacagcc

SEQ ID NO: 620 - RNA, 21 nt, Rattus norvegicus
cugaccuaugaauugacagcc

SEQ ID NO: 621 - RNA, 21 nt, Xenopus tropicalis
augaccuaugaauugacagcc

SEQ ID NO: 622 - RNA, 23 nt, Bos taurus
cugaccuaugaauugacagccag

SEQ ID NO: 623 - RNA, 21 nt, Danio rerio
augaccuaugaauugacagcc

SEQ ID NO: 624 - RNA, 21 nt, Fugu rubripes
augaccuaugaauugacagcc

SEQ ID NO: 625 - RNA, 21 nt, Tetraodon nigroviridis
augaccuaugaauugacagcc

SEQ ID NO: 626 - RNA, 21 nt, Mus musculus
augaccuaugauuugacagac

SEQ ID NO: 627 - RNA, 21 nt, Rattus norvegicus
augaccuaugauuugacagac

SEQ ID NO: 628 - RNA, 21 nt, Macaca mulatta
augaccuaugaauugacagac

SEQ ID NO: 629 - RNA, 21 nt, Macaca nemestrina
augaccuaugaauugacagac

SEQ ID NO: 630 - RNA, 21 nt, Gorilla gorilla
augaccuaugaauugacagac

SEQ ID NO: 631 - RNA, 21 nt, Pan troglodytes
augaccuaugaauugacagac

SEQ ID NO: 632 - RNA, 21 nt, Pongo pygmaeus
augaccuaugaauugacagac

SEQ ID NO: 633 - RNA, 21 nt, Gallus gallus
augaccuaugaauugacagac

SEQ ID NO: 634 - RNA, 21 nt, Xenopus tropicalis
augaccuaugaaaugacagcc

SEQ ID NO: 635 - RNA, 22 nt, Bos taurus
augaccuaugaauugacagaca

SEQ ID NO: 636 - RNA, 22 nt, Rattus norvegicus
aacuggccuacaaagucccagu

SEQ ID NO: 637 - RNA, 21 nt, Monodelphis domestica
aacuggccuacaaagucccag

SEQ ID NO: 638 - RNA, 24 nt, Gallus gallus
aacuggcccacaaagucccgcuuu

SEQ ID NO: 639 - RNA, 24 nt, Xenopus tropicalis
aacuggcccgcaaagucccgcuuc

SEQ ID NO: 640 - RNA, 22 nt, Bos taurus
aacuggccuacaaagucccagu

SEQ ID NO: 641 - RNA, 22 nt, Danio rerio
aacuggccuacaaagucccagu

SEQ ID NO: 642 - RNA, 22 nt, Fugu rubripes
aacuggccuacaaagucccagu

SEQ ID NO: 643 - RNA, 22 nt, Tetraodon nigroviridis
aacuggccuacaaagucccagu

SEQ ID NO: 644 - RNA, 22 nt, Mus musculus
aacuggcccacaaagucccgcu

SEQ ID NO: 645 - RNA, 22 nt, Danio rerio
aacuggcccgcaaagucccgcu

SEQ ID NO: 646 - RNA, 21 nt, Mus musculus
uagcagcacagaaauauuggc

SEQ ID NO: 647 - RNA, 21 nt, Rattus norvegicus
uagcagcacagaaauauuggc

SEQ ID NO: 648 - RNA, 21 nt, Gorilla gorilla
uagcagcacagaaauauuggc

SEQ ID NO: 649 - RNA, 21 nt, Pan paniscus
uagcagcacagaaauauuggc

SEQ ID NO: 650 - RNA, 22 nt, Bos taurus
uagcagcacagaaauauuggca

SEQ ID NO: 651 - RNA, 22 nt, Mus musculus
cagcagcacacugugguuugua

SEQ ID NO: 652 - RNA, 22 nt, Rattus norvegicus
cagcagcacacugugguuugua

SEQ ID NO: 653 - RNA, 22 nt, Bos taurus
cagcagcacacugugguuugua

SEQ ID NO: 654 - RNA, 22 nt, Mus musculus
uagguaguuucauguuguuggg

SEQ ID NO: 655 - RNA, 22 nt, Rattus norvegicus
uagguaguuucauguuguuggg

SEQ ID NO: 656 - RNA, 22 nt, Ateles geoffroyi
uagguaguuucauguuguuggg

SEQ ID NO: 657 - RNA, 22 nt, Lagothrix lagotricha
uagguaguuucauguuguuggg

SEQ ID NO: 658 - RNA, 21 nt, Macaca mulatta
uagguaguuucauguuguugg

SEQ ID NO: 659 - RNA, 21 nt, Gorilla gorilla
uagguaguuucauguuguugg

SEQ ID NO: 660 - RNA, 22 nt, Pan paniscus
uagguaguuucauguuguuggg

SEQ ID NO: 661 - RNA, 22 nt, Pan troglodytes
uagguaguuucauguuguuggg

SEQ ID NO: 662 - RNA, 21 nt, Pongo pygmaeus
uagguaguuucauguuguugg

SEQ ID NO: 663 - RNA, 21 nt, Gallus gallus
uagguaguuucauguuguugg

SEQ ID NO: 664 - RNA, 21 nt, Xenopus tropicalis
uagguaguuucauguuguugg

SEQ ID NO: 665 - RNA, 21 nt, Sus scrofa
uagguaguuucauguuguugg

SEQ ID NO: 666 - RNA, 22 nt, Danio rerio
uagguaguuucauguuguuggg

SEQ ID NO: 667 - RNA, 22 nt, Fugu rubripes
uagguaguuucauguuguuggg

SEQ ID NO: 668 - RNA, 22 nt, Tetraodon nigroviridis
uagguaguuucauguuguuggg

SEQ ID NO: 669 - RNA, 22 nt, Mus musculus
uagguaguuuccuguuguuggg

SEQ ID NO: 670 - RNA, 22 nt, Rattus norvegicus
uagguaguuuccuguuguuggg

SEQ ID NO: 671 - RNA, 21 nt, Monodelphis domestica
uagguaguuuccuguuguugg

SEQ ID NO: 672 - RNA, 21 nt, Xenopus tropicalis
uagguaguuuuauguuguugg

SEQ ID NO: 673 - RNA, 22 nt, Danio rerio
uagguaguuucaaguuguuggg

SEQ ID NO: 674 - RNA, 22 nt, Fugu rubripes
uagguaguuucaaguuguuggg

SEQ ID NO: 675 - RNA, 22 nt, Tetraodon nigroviridis
uagguaguuucaaguuguuggg

SEQ ID NO: 676 - RNA, 22 nt, Mus musculus
uucaccaccuucuccacccagc

SEQ ID NO: 677 - RNA, 22 nt, Ateles geoffroyi
uucaccaccuucuccacccagc

SEQ ID NO: 678 - RNA, 22 nt, Macaca nemestrina
uucaccaccuucuccacccggc

SEQ ID NO: 679 - RNA, 22 nt, Pan paniscus
uucaccaccuucuccacccagc

SEQ ID NO: 680 - RNA, 22 nt, Pan troglodytes
uucaccaccuucuccacccagc

SEQ ID NO: 681 - RNA, 22 nt, Pongo pygmaeus
uucaccaccuucuccacccagc

SEQ ID NO: 682 - RNA, 22 nt, Xenopus tropicalis
uacaguagucugcacauugguu

SEQ ID NO: 683 - RNA, 22 nt, Danio rerio
uacaguagucugcacauugguu

SEQ ID NO: 684 - RNA, 22 nt, Mus musculus
uaacacugucugguaacgaugu

SEQ ID NO: 685 - RNA, 22 nt, Rattus norvegicus
uaacacugucugguaacgaugu

SEQ ID NO: 686 - RNA, 22 nt, Monodelphis domestica
uaacacugucugguaacgaugu

SEQ ID NO: 687 - RNA, 22 nt, Gallus gallus
uaacacugucugguaacgaugu

SEQ ID NO: 688 - RNA, 22 nt, Xenopus tropicalis
uaacacugucugguaacgaugu

SEQ ID NO: 689 - RNA, 23 nt, Bos taurus
uaacacugucugguaacgauguu

SEQ ID NO: 690 - RNA, 22 nt, Danio rerio
uaacacugucugguaacgaugu

SEQ ID NO: 691 - RNA, 22 nt, Fugu rubripes
uaacacugucugguaacgaugu

SEQ ID NO: 692 - RNA, 22 nt, Tetraodon nigroviridis
uaacacugucugguaacgaugu

SEQ ID NO: 693 - RNA, 22 nt, Mus musculus
uucccuuugucauccuaugccu

SEQ ID NO: 694 - RNA, 22 nt, Rattus norvegicus
uucccuuugucauccuaugccu

SEQ ID NO: 695 - RNA, 22 nt, Saguinus labiatus
uucccuuugucauccuaugccu

SEQ ID NO: 696 - RNA, 22 nt, Macaca nemestrina
uucccuuugucauccuaugccu

SEQ ID NO: 697 - RNA, 22 nt, Gorilla gorilla
uucccuuugucauccuaugccu

SEQ ID NO: 698 - RNA, 22 nt, Pan paniscus
uucccuuugucauccuaugccu

SEQ ID NO: 699 - RNA, 22 nt, Pan troglodytes
uucccuuugucauccuaugccu

SEQ ID NO: 700 - RNA, 22 nt, Pongo pygmaeus
uucccuuugucauccuaugccu

SEQ ID NO: 701 - RNA, 22 nt, Monodelphis domestica
uucccuuugucauccuaugccu

SEQ ID NO: 702 - RNA, 22 nt, Gallus gallus
uucccuuugucauccuaugccu

SEQ ID NO: 703 - RNA, 22 nt, Xenopus tropicalis
uucccuuugucauccuaugccu

SEQ ID NO: 704 - RNA, 22 nt, Bos taurus
uucccuuugucauccuaugccu

SEQ ID NO: 705 - RNA, 22 nt, Sus scrofa
uucccuuugucauccuaugccu

SEQ ID NO: 706 - RNA, 22 nt, Danio rerio
uucccuuugucauccuaugccu

SEQ ID NO: 707 - RNA, 22 nt, Fugu rubripes
uucccuuugucauccuaugccu

SEQ ID NO: 708 - RNA, 22 nt, Tetraodon nigroviridis
uucccuuugucauccuaugccu

SEQ ID NO: 709 - RNA, 22 nt, Mus musculus
uucccuuugucauccuuugccu

SEQ ID NO: 710 - RNA, 22 nt, Rattus norvegicus
uucccuuugucauccuuugccu

SEQ ID NO: 711 - RNA, 22 nt, Macaca mulatta
uucccuuugucauccuuugccu

SEQ ID NO: 712 - RNA, 22 nt, Macaca nemestrina
uucccuuugucauccuucgccu

SEQ ID NO: 713 - RNA, 22 nt, Pongo pygmaeus
uucccuuugucauccuucgccu

SEQ ID NO: 714 - RNA, 22 nt, Gallus gallus
uucccuuugucauccuaugccu

SEQ ID NO: 715 - RNA, 22 nt, Mus musculus
uccuucauuccaccggagucug

SEQ ID NO: 716 - RNA, 22 nt, Rattus norvegicus
uccuucauuccaccggagucug

SEQ ID NO: 717 - RNA, 22 nt, Ateles geoffroyi
uccuucauuccaccggagucug

SEQ ID NO: 718 - RNA, 22 nt, Lagothrix lagotricha
uccuucauuccaccggagucug

SEQ ID NO: 719 - RNA, 22 nt, Macaca nemestrina
uccuucauuccaccggagucug

SEQ ID NO: 720 - RNA, 22 nt, Gorilla gorilla
uccuucauuccaccggagucug

SEQ ID NO: 721 - RNA, 22 nt, Pan paniscus
uccuucauuccaccggagucug

SEQ ID NO: 722 - RNA, 22 nt, Pan troglodytes
uccuucauuccaccggagucug

SEQ ID NO: 723 - RNA, 22 nt, Gallus gallus
uccuucauuccaccggagucug

SEQ ID NO: 724 - RNA, 22 nt, Gallus gallus
cccuucauuccaccggaaucug

SEQ ID NO: 725 - RNA, 22 nt, Xenopus tropicalis
uccuucauuccaccggagucug

SEQ ID NO: 726 - RNA, 22 nt, Bos taurus
uccuucauuccaccggagucug

SEQ ID NO: 727 - RNA, 22 nt, Sus scrofa
uccuucauuccaccggagucug

SEQ ID NO: 728 - RNA, 22 nt, Danio rerio
uccuucauuccaccggagucug

SEQ ID NO: 729 - RNA, 22 nt, Fugu rubripes
uccuucauuccaccggagucug

SEQ ID NO: 730 - RNA, 22 nt, Tetraodon nigroviridis
uccuucauuccaccggagucug

SEQ ID NO: 731 - RNA, 22 nt, Mus musculus
auaagacgagcaaaaagcuugu

SEQ ID NO: 732 - RNA, 22 nt, Rattus norvegicus
auaagacgagcaaaaagcuugu

SEQ ID NO: 733 - RNA, 22 nt, Monodelphis domestica
auaagacgagcaaaaagcucgu

SEQ ID NO: 734 - RNA, 22 nt, Xenopus tropicalis
auaagacgagcauaaagcuugu

SEQ ID NO: 735 - RNA, 21 nt, Mus musculus
agcuacaucuggcuacugggu

SEQ ID NO: 736 - RNA, 21 nt, Rattus norvegicus
agcuacaucuggcuacugggu

SEQ ID NO: 737 - RNA, 24 nt, Ateles geoffroyi
agcuacaucuggcuacugggucuc

SEQ ID NO: 738 - RNA, 24 nt, Gallus gallus
agcuacaucuggcuacugggucuc

SEQ ID NO: 739 - RNA, 24 nt, Xenopus tropicalis
agcuacaucuggcuacugggucuc

SEQ ID NO: 740 - RNA, 21 nt, Bos taurus
agcuacaucuggcuacugggu

SEQ ID NO: 741 - RNA, 24 nt, Danio rerio
agcuacaucuggcuacugggucuc

SEQ ID NO: 742 - RNA, 24 nt, Fugu rubripes
agcuacaucuggcuacugggucuc

SEQ ID NO: 743 - RNA, 24 nt, Tetraodon nigroviridis
agcuacaucuggcuacugggucuc

SEQ ID NO: 744 - RNA, 23 nt, Mus musculus
acuuuaacaugggaaugcuuucu

SEQ ID NO: 745 - RNA, 23 nt, Gallus gallus
acuuuaacauggaggugcuuucu

SEQ ID NO: 746 - RNA, 20 nt, Mus musculus
ccacugccccaggugcugcu

SEQ ID NO: 747 - RNA, 22 nt, Rattus norvegicus
ccacugccccaggugcugcugg

SEQ ID NO: 748 - RNA, 23 nt, Mus musculus
cgcauccccuagggcauuggugu

SEQ ID NO: 749 - RNA, 23 nt, Rattus norvegicus
cgcauccccuagggcauuggugu

SEQ ID NO: 750 - RNA, 22 nt, Mus musculus
aacacacccagcuaaccuuuuu

SEQ ID NO: 751 - RNA, 22 nt, Rattus norvegicus
aacacacccagcuaaccuuuuu

SEQ ID NO: 752 - RNA, 21 nt, Rattus norvegicus
gccccugggccuauccuagaa

SEQ ID NO: 753 - RNA, 21 nt, Bos taurus
gccccugggccuauccuagaa

SEQ ID NO: 754 - RNA, 25 nt, Bos taurus
ucucacacagaaaucgcacccaucu

SEQ ID NO: 755 - RNA, 23 nt, Mus musculus
ugucugcccgagugccugccucu

SEQ ID NO: 756 - RNA, 23 nt, Rattus norvegicus
ugucugccugagugccugccucu

SEQ ID NO: 757 - RNA, 21 nt, Mus musculus
aucauagaggaacauccacuu

SEQ ID NO: 758 - RNA, 22 nt, Mus musculus
aucacacaaaggcaacuuuugu

SEQ ID NO: 759 - RNA, 22 nt, Rattus norvegicus
aucacacaaaggcaacuuuugg

SEQ ID NO: 760 - RNA, 22 nt, Bos taurus
ugguugaccauagaacaugcgc

SEQ ID NO: 761 - RNA, 22 nt, Mus musculus
gaaguuguucgugguggauucg

SEQ ID NO: 762 - RNA, 22 nt, Rattus norvegicus
gaaguuguucgugguggauucg

SEQ ID NO: 763 - RNA, 23 nt, Rattus norvegicus
acuucaccugguccacuagccgu

SEQ ID NO: 764 - RNA, 23 nt, Rattus norvegicus
agcucggucugaggccccucagu

SEQ ID NO: 765 - RNA, 21 nt, Mus musculus
ugucuugcaggccgucaugca

SEQ ID NO: 766 - RNA, 21 nt, Rattus norvegicus
ugucuugcaggccgucaugca

SEQ ID NO: 767 - RNA, 23 nt, Ovis aries
ugucuugcaggccgucaugcagg

SEQ ID NO: 768 - RNA, 22 nt, Mus musculus
aucaugaugggcuccucggugu

SEQ ID NO: 769 - RNA, 22 nt, Rattus norvegicus
aucaugaugggcuccucggugu

SEQ ID NO: 770 - RNA, 20 nt, Mus musculus
aggcaguguuguuagcuggc

SEQ ID NO: 771 - RNA, 21 nt, Mus musculus
aggcagugcauugcuagcugg

SEQ ID NO: 772 - RNA, 22 nt, Rattus norvegicus
uggcaguguauuguuagcuggu

SEQ ID NO: 773 - RNA, 22 nt, Canis familiaris
uggcaguguauuguuagcuggu

SEQ ID NO: 774 - RNA, 22 nt, Monodelphis domestica
uggcaguguauuguuagcuggu

SEQ ID NO: 775 - RNA, 21 nt, Gallus gallus
uggcaguguauguuagcuggu

SEQ ID NO: 776 - RNA, 22 nt, Xenopus tropicalis
aggcaguguaauguuagcuggu

SEQ ID NO: 777 - RNA, 22 nt, Mus musculus
aaaccguuaccauuacugaguu

SEQ ID NO: 778 - RNA, 22 nt, Rattus norvegicus
aaaccguuaccauuacugaguu

SEQ ID NO: 779 - RNA, 22 nt, Monodelphis domestica
aaaccguuaccauuacuguguu

SEQ ID NO: 780 - RNA, 23 nt, Gallus gallus
aaaccguuaccauuacugaguuu

SEQ ID NO: 781 - RNA, 22 nt, Xenopus tropicalis
aaaccguuaccauuacugaguu

SEQ ID NO: 782 - RNA, 22 nt, Danio rerio
aaaccguuaccauuacugaguu

SEQ ID NO: 783 - RNA, 22 nt, Mus musculus
uguuugcagaggaaacugagac

SEQ ID NO: 784 - RNA, 22 nt, Rattus norvegicus
uaugugccuuuggacuacaucg

SEQ ID NO: 785 - RNA, 22 nt, Gallus gallus
uaugugcccuuggacuacaucg

SEQ ID NO: 786 - RNA, 22 nt, Xenopus tropicalis
uaugugcccuuggacuacaucg

SEQ ID NO: 787 - RNA, 21 nt, Bos taurus
uaugugccuuuggacuacauc

SEQ ID NO: 788 - RNA, 22 nt, Danio rerio
uaugugcccuuggacuacaucg

SEQ ID NO: 789 - RNA, 22 nt, Fugu rubripes
uaugugcccuuggacuacaucg

SEQ ID NO: 790 - RNA, 22 nt, Tetraodon nigroviridis
uaugugcccuuggacuacaucg

SEQ ID NO: 791 - RNA, 23 nt, Rattus norvegicus
ucacuccuccccucccgucuugu

SEQ ID NO: 792 - RNA, 23 nt, Gallus gallus
agugacaucauauguacggcugc

SEQ ID NO: 793 - RNA, 23 nt, Xenopus tropicalis
agugacaucauauguacggcugc

SEQ ID NO: 794 - RNA, 23 nt, Danio rerio
agugacaucauauguacggcugc

SEQ ID NO: 795 - RNA, 23 nt, Fugu rubripes
agugacaucauauguacggcugc

SEQ ID NO: 796 - RNA, 23 nt, Tetraodon nigroviridis
agugacaucauauguacggcugc

SEQ ID NO: 797 - RNA, 22 nt, Mus musculus
aguggggaacccuuccaugagg

SEQ ID NO: 798 - RNA, 22 nt, Mus musculus
ugaaacauacacgggaaaccuc

SEQ ID NO: 799 - RNA, 21 nt, Rattus norvegicus
ugaaacauacacgggaaaccu

SEQ ID NO: 800 - RNA, 22 nt, Rattus norvegicus
aauccuuugucccugggugaaa

SEQ ID NO: 801 - RNA, 22 nt, Mus musculus
agacccuggucugcacucuauc

SEQ ID NO: 802 - RNA, 20 nt, Rattus norvegicus
gucaacacuugcugguuucc

SEQ ID NO: 803 - RNA, 20 nt, Homo sapiens
aggaauguuccuucuuugcc

SEQ ID NO: 804 - RNA, 23 nt, Homo sapiens
uacccuguagaaccgaauuugug

SEQ ID NO: 805 - RNA, 23 nt, Homo sapiens
caaagugcuuacagugcagguag

SEQ ID NO: 806 - RNA, 22 nt, Homo sapiens
caaagugccucccuuuagagug

SEQ ID NO: 807 - RNA, 22 nt, Homo sapiens
gaaagugcuuccuuuuagaggc

SEQ ID NO: 808 - RNA, 23 nt, Mus musculus
caaagugcuuacagugcagguag

SEQ ID NO: 809 - RNA, 23 nt, Mus musculus
caaagugcuguucgugcagguag

SEQ ID NO: 810 - RNA, 23 nt, Rattus norvegicus
caaagugcuuacagugcagguag

SEQ ID NO: 811 - RNA, 23 nt, Rattus norvegicus
caaagugcuguucgugcagguag

SEQ ID NO: 812 - RNA, 23 nt, Xenopus tropicalis
caaagugcuguucgugcagguag

SEQ ID NO: 813 - RNA, 22 nt, Bos taurus
caaagugcuguucgugcaggua

SEQ ID NO: 814 - RNA, 22 nt, Danio rerio
caaagugcuuacagugcaggua

SEQ ID NO: 815 - RNA, 22 nt, Danio rerio
aaaagugcuguuugugcaggua

SEQ ID NO: 816 - RNA, 22 nt, Fugu rubripes
caaagugcuuacagugcaggua

SEQ ID NO: 817 - RNA, 22 nt, Tetraodon nigroviridis
caaagugcuuacagugcaggua

SEQ ID NO: 818 - RNA, 22 nt, Homo sapiens
uauugcacuugucccggccugu

SEQ ID NO: 819 - RNA, 22 nt, Homo sapiens
uauugcacucgucccggccucc

SEQ ID NO: 820 - RNA, 22 nt, Homo sapiens
aauugcacgguauccaucugua

SEQ ID NO: 821 - RNA, 22 nt, Homo sapiens
aauugcacuuuagcaaugguga

SEQ ID NO: 822 - RNA, 21 nt, Mus musculus
uauugcacuugucccggccug

SEQ ID NO: 823 - RNA, 22 nt, Mus musculus
uauugcacucgucccggccucc

SEQ ID NO: 824 - RNA, 22 nt, Mus musculus
aauugcacgguauccaucugua

SEQ ID NO: 825 - RNA, 22 nt, Mus musculus
aauugcacuuuagcaaugguga

SEQ ID NO: 826 - RNA, 21 nt, Rattus norvegicus
uauugcacuugucccggccug

SEQ ID NO: 827 - RNA, 22 nt, Rattus norvegicus
uauugcacucgucccggccucc

SEQ ID NO: 828 - RNA, 22 nt, Rattus norvegicus
aauugcacgguauccaucugua

SEQ ID NO: 829 - RNA, 22 nt, Ateles geoffroyi
uauugcacuugucccggccugu

SEQ ID NO: 830 - RNA, 22 nt, Lagothrix lagotricha
uauugcacuugucccggccugu

SEQ ID NO: 831 - RNA, 22 nt, Saguinus labiatus
uauugcacuugucccggccugu

SEQ ID NO: 832 - RNA, 22 nt, Macaca mulatta
uauugcacuugucccggccugu

SEQ ID NO: 833 - RNA, 22 nt, Macaca nemestrina
uauugcacuugucccggccugu

SEQ ID NO: 834 - RNA, 22 nt, Gorilla gorilla
uauugcacuugucccggccugu

SEQ ID NO: 835 - RNA, 22 nt, Pan paniscus
uauugcacuugucccggccugu

SEQ ID NO: 836 - RNA, 22 nt, Pan troglodytes
uauugcacuugucccggccugu

SEQ ID NO: 837 - RNA, 22 nt, Pongo pygmaeus
uauugcacuugucccggccugu

SEQ ID NO: 838 - RNA, 22 nt, Lemur catta
uauugcacuugucccggccugu

SEQ ID NO: 839 - RNA, 21 nt, Monodelphis domestica
uauugcacuugucccggccug

SEQ ID NO: 840 - RNA, 21 nt, Gallus gallus
uauugcacuugucccggccug

SEQ ID NO: 841 - RNA, 21 nt, Gallus gallus
aauugcacuuuagcaauggug

SEQ ID NO: 842 - RNA, 21 nt, Xenopus tropicalis
uauugcacuugucccggccug

SEQ ID NO: 843 - RNA, 21 nt, Xenopus tropicalis
uauugcacucgucccggccuc

SEQ ID NO: 844 - RNA, 21 nt, Xenopus tropicalis
aauugcacgguauccaucugu

SEQ ID NO: 845 - RNA, 22 nt, Xenopus tropicalis
aauugcacuguagcaaugguga

SEQ ID NO: 846 - RNA, 22 nt, Bos taurus
uauugcacuugucccggccugu

SEQ ID NO: 847 - RNA, 22 nt, Danio rerio
uauugcacuugucccggccugu

SEQ ID NO: 848 - RNA, 22 nt, Danio rerio
uauugcacucgucccggccucc

SEQ ID NO: 849 - RNA, 22 nt, Danio rerio
aauugcacgguauccaucugua

SEQ ID NO: 850 - RNA, 22 nt, Fugu rubripes
uauugcacuugucccggccugu

SEQ ID NO: 851 - RNA, 22 nt, Tetraodon nigroviridis
uauugcacuugucccggccugu

SEQ ID NO: 852 - RNA, 21 nt, Homo sapiens
uucacaguggcuaaguuccgc

SEQ ID NO: 853 - RNA, 23 nt, Homo sapiens
aaggagcuuacaaucuagcuggg

SEQ ID NO: 854 - RNA, 23 nt, Mus musculus
aaggagcuuacaaucuagcuggg

SEQ ID NO: 855 - RNA, 23 nt, Rattus norvegicus
aaggagcuuacaaucuagcuggg

SEQ ID NO: 856 - RNA, 23 nt, Homo sapiens
uagcaccauuugaaaucaguguu

SEQ ID NO: 857 - RNA, 22 nt, Homo sapiens
uguaaacauccucgacuggaag

SEQ ID NO: 858 - RNA, 22 nt, Homo sapiens
uguaaacauccccgacuggaag

SEQ ID NO: 859 - RNA, 22 nt, Homo sapiens
uguaaacauccuugacuggaag

SEQ ID NO: 860 - RNA, 23 nt, Mus musculus
uguaaacaauuccuaggcaaugu

SEQ ID NO: 861 - RNA, 23 nt, Rattus norvegicus
uguaaacaauuccuaggcaaugu

SEQ ID NO: 862 - RNA, 23 nt, Homo sapiens
uaagugcuuccauguuuugguga

SEQ ID NO: 863 - RNA, 23 nt, Homo sapiens
uaagugcuuccauguuuuaguag

SEQ ID NO: 864 - RNA, 23 nt, Homo sapiens
uaagugcuuccauguuucagugg

SEQ ID NO: 865 - RNA, 22 nt, Homo sapiens
aaagugcuucccuuuggacugu

SEQ ID NO: 866 - RNA, 21 nt, Homo sapiens
aaagugcuuccuuuuagaggg

SEQ ID NO: 867 - RNA, 22 nt, Homo sapiens
aaagugcuuccuuuuagagggu

SEQ ID NO: 868 - RNA, 22 nt, Homo sapiens
aaagugcuucucuuuggugggu

SEQ ID NO: 869 - RNA, 21 nt, Homo sapiens
aaagugcuuccuuuuugaggg

SEQ ID NO: 870 - RNA, 22 nt, Mus musculus
aaagugcuuccacuuugugugc

SEQ ID NO: 871 - RNA, 22 nt, Mus musculus
aaagugcuucccuuuugugugu

SEQ ID NO: 872 - RNA, 23 nt, Mus musculus
aaagugcuacuacuuuugagucu

SEQ ID NO: 873 - RNA, 23 nt, Rattus norvegicus
aaagugcuuccacuuugugugcc

SEQ ID NO: 874 - RNA, 22 nt, Xenopus laevis
aaagugcuuucuguuuugggcg

SEQ ID NO: 875 - RNA, 22 nt, Xenopus laevis
uaagugcucucuaguucgguug

SEQ ID NO: 876 - RNA, 22 nt, Xenopus tropicalis
uaagugcucucuaguucgguug

SEQ ID NO: 877 - RNA, 22 nt, Danio rerio
uaagugcuauuuguugggguag

SEQ ID NO: 878 - RNA, 23 nt, Danio rerio
aaagugcuaucaaguugggguag

SEQ ID NO: 879 - RNA, 22 nt, Danio rerio
uaagugcuucucuuugggguag

SEQ ID NO: 880 - RNA, 22 nt, Danio rerio
uaagugcuauuuguuggcguag

SEQ ID NO: 881 - RNA, 22 nt, Danio rerio
aaagugcuaucaaauuggggua

SEQ ID NO: 882 - RNA, 23 nt, Homo sapiens
cagugcaaugauauugucaaagc

SEQ ID NO: 883 - RNA, 23 nt, Homo sapiens
uagugcaauauugcuuauagggu

SEQ ID NO: 884 - RNA, 21 nt, Mus musculus
cagugcaauuaaaagggggaa

SEQ ID NO: 885 - RNA, 22 nt, Danio rerio
uagugcaauauugcuaauaggg

SEQ ID NO: 886 - RNA, 22 nt, Danio rerio
uagugcaauauugcuuauaggg

SEQ ID NO: 887 - RNA, 22 nt, Homo sapiens
ugagaacugaauuccauaggcu

SEQ ID NO: 888 - RNA, 22 nt, Mus musculus
cagcagcaauucauguuuugga

SEQ ID NO: 889 - RNA, 22 nt, Rattus norvegicus
cagcagcaauucauguuuugga

SEQ ID NO: 890 - RNA, 22 nt, Danio rerio
aagcagcacaucaauauuggca

SEQ ID NO: 891 - RNA, 22 nt, Danio rerio
aagcagcacauaaauacuggag

SEQ ID NO: 892 - RNA, 22 nt, Homo sapiens
uaacacugucugguaaagaugg

SEQ ID NO: 893 - RNA, 22 nt, Mus musculus
uaacacugucugguaaagaugg

SEQ ID NO: 894 - RNA, 22 nt, Rattus norvegicus
uaacacugucugguaaagaugg

SEQ ID NO: 895 - RNA, 22 nt, Monodelphis domestica
uaacacugucugguaaagaugc

SEQ ID NO: 896 - RNA, 22 nt, Danio rerio
uaacacugucugguaacgaugc

SEQ ID NO: 897 - RNA, 22 nt, Homo sapiens
auaagacgaacaaaagguuugu

SEQ ID NO: 898 - RNA, 21 nt, Danio rerio
guaagacgaacaaaaaguuuu

SEQ ID NO: 899 - RNA, 23 nt, Homo sapiens
agcuacauugucugcuggguuuc

SEQ ID NO: 900 - RNA, 23 nt, Mus musculus
agcuacauugucugcuggguuuc

SEQ ID NO: 901 - RNA, 23 nt, Rattus norvegicus
agcuacauugucugcuggguuuc

SEQ ID NO: 902 - RNA, 23 nt, Macaca mulatta
agcuacauugucugcuggguuuc

SEQ ID NO: 903 - RNA, 23 nt, Gorilla gorilla
agcuacauugucugcuggguuuc

SEQ ID NO: 904 - RNA, 23 nt, Pan paniscus
agcuacauugucugcuggguuuc

SEQ ID NO: 905 - RNA, 23 nt, Pongo pygmaeus
agcuacauugucugcuggguuuc

SEQ ID NO: 906 - RNA, 23 nt, Monodelphis domestica
agcuacauugucugcuggguuuc

SEQ ID NO: 907 - RNA, 23 nt, Gallus gallus
agcuacauugucugcuggguuuc

SEQ ID NO: 908 - RNA, 23 nt, Xenopus tropicalis
agcuacauugucugcuggguuuc

SEQ ID NO: 909 - RNA, 22 nt, Bos taurus
agcuacauugucugcuggguuu

SEQ ID NO: 910 - RNA, 23 nt, Danio rerio
agcuacauugucugcuggguuuc

SEQ ID NO: 911 - RNA, 23 nt, Fugu rubripes
agcuacauugucugcuggguuuc

SEQ ID NO: 912 - RNA, 23 nt, Tetraodon nigroviridis
agcuacauugucugcuggguuuc

SEQ ID NO: 913 - RNA, 22 nt, Homo sapiens
acuuuaacauggaggcacuugc

SEQ ID NO: 914 - RNA, 22 nt, Homo sapiens
aacacaccuauucaaggauuca

SEQ ID NO: 915 - RNA, 22 nt, Mus musculus
aacacaccuguucaaggauuca

SEQ ID NO: 916 - RNA, 19 nt, Homo sapiens
agguugacauacguuuccc

SEQ ID NO: 917 - RNA, 22 nt, Homo sapiens
aggcaguguauuguuagcuggc

SEQ ID NO: 918 - RNA, 20 nt, Mus musculus
aggcagugcgaccuggcucg

SEQ ID NO: 919 - RNA, 20 nt, Homo sapiens
uaaggcacgcggugaaugcc

SEQ ID NO: 920 - RNA, 20 nt, Mus musculus
uaaggcacgcggugaaugcc

SEQ ID NO: 921 - RNA, 20 nt, Rattus norvegicus
uaaggcacgcggugaaugcc

SEQ ID NO: 922 - RNA, 22 nt, Danio rerio
uaaggcacgcggugaaugccaa

SEQ ID NO: 923 - RNA, 22 nt, Fugu rubripes
uaaggcacgcggugaaugccaa

SEQ ID NO: 924 - RNA, 22 nt, Tetraodon nigroviridis
uaaggcacgcggugaaugccaa

SEQ ID NO: 925 - RNA, 23 nt, Homo sapiens
guuugcacgggugggccuugucu

SEQ ID NO: 926 - RNA, 22 nt, Homo sapiens
gaaagcgcuucccuuugcugga

SEQ ID NO: 927 - RNA, 24 nt, Homo sapiens
acaaagugcuucccuuuagagugu

SEQ ID NO: 928 - RNA, 20 nt, Homo sapiens
cuacaaagggaagcccuuuc

SEQ ID NO: 929 - RNA, 21 nt, Homo sapiens
cuccagagggaaguacuuucu

SEQ ID NO: 930 - RNA, 22 nt, Homo sapiens
cucuagagggaagcacuuucug

SEQ ID NO: 931 - RNA, 22 nt, Homo sapiens
cucuagagggaagcgcuuucug

SEQ ID NO: 932 - RNA, 22 nt, Homo sapiens
cucuagagggaagcacuuucuc

SEQ ID NO: 933 - RNA, 22 nt, Homo sapiens
cucuagagggaagcgcuuucug

SEQ ID NO: 934 - RNA, 22 nt, Homo sapiens
cucuagagggaagcgcuuucug

SEQ ID NO: 935 - RNA, 22 nt, Homo sapiens
cucuagagggaagcgcuuucug

SEQ ID NO: 936 - RNA, 22 nt, Homo sapiens
cucuagagggaagcacuuucug

SEQ ID NO: 937 - RNA, 22 nt, Homo sapiens
cucuagagggaagcgcuuucug

SEQ ID NO: 938 - RNA, 22 nt, Homo sapiens
cucuagagggaagcgcuuucug

SEQ ID NO: 939 - RNA, 20 nt, Homo sapiens
cugcaaagggaagcccuuuc

SEQ ID NO: 940 - RNA, 71 nt, Homo sapiens
ugggaaacauacuucuuuauaugcccauauggaccugcuaagcuauggaauguaaagaaguauguaucuca

SEQ ID NO: 941 - RNA, 85 nt, Homo sapiens
accuacucagaguacauacuucuuuauguacccauaugaacauacaaugcuauggaauguaaagaaguauguauuuuugguaggc

SEQ ID NO: 942 - RNA, 86 nt, Homo sapiens
ugcuucccgaggccacaugcuucuuuauauccccauauggauuacuuugcuauggaauguaaggaagugugugguuucggcaagug

SEQ ID NO: 943 - RNA, 110 nt, Homo sapiens
uuggauguuggccuaguucuguguggaagacuagugauuuuguuguuuuuagauaacuaaaucgacaacaaaucacagucugccauauggcacaggccaugccucuacag

SEQ ID NO: 944 - RNA, 110 nt, Homo sapiens
cuggauacagaguggaccggcuggccccaucuggaagacuagugauuuuguuguugucuuacugcgcucaacaacaaaucccagucuaccuaauggugccagccaucgca

SEQ ID NO: 945 - RNA, 110 nt, Homo sapiens
agauuagaguggcuguggucuagugcuguguggaagacuagugauuuuguuguucugauguacuacgacaacaagucacagccggccucauagcgcagacucccuucgac

SEQ ID NO: 946 - RNA, 110 nt, Homo sapiens
gaucugucugucuucuguauauacccuguagauccgaauuuguguaaggaauuuuguggucacaaauucguaucuaggggaauauguaguugacauaaacacuccgcucu

SEQ ID NO: 947 - RNA, 71 nt, Homo sapiens
uguucuaaggugcaucuagugcagauagugaaguagauuagcaucuacugcccuaagugcuccuucuggca

SEQ ID NO: 948 - RNA, 71 nt, Homo sapiens
uguguuaaggugcaucuagugcaguuagugaagcagcuuagaaucuacugcccuaaaugccccuucuggca

SEQ ID NO: 949 - RNA, 69 nt, Homo sapiens
aguaccaaagugcucauagugcagguaguuuuggcaugacucuacuguaguaugggcacuuccaguacu

SEQ ID NO: 950 - RNA, 81 nt, Homo sapiens
ccuuggccauguaaaagugcuuacagugcagguagcuuuuugagaucuacugcaauguaagcacuucuuacauuaccaugg

SEQ ID NO: 951 - RNA, 82 nt, Homo sapiens
ccugccggggcuaaagugcugacagugcagauagugguccucuccgugcuaccgcacuguggguacuugcugcuccagcagg

SEQ ID NO: 952 - RNA, 68 nt, Homo sapiens
cuccggugccuacugagcugauaucaguucucauuuuacacacuggcucaguucagcaggaacaggag

SEQ ID NO: 953 - RNA, 73 nt, Homo sapiens
cucugccucccgugccuacugagcugaaacacaguugguuuguguacacuggcucaguucagcaggaacaggg

SEQ ID NO: 954 - RNA, 84 nt, Homo sapiens
ggccaguguugagaggcggagacuugggcaauugcuggacgcugcccugggcauugcacuugucucggucugacagugccggcc

SEQ ID NO: 955 - RNA, 70 nt, Homo sapiens
ggagauauugcacauuacuaaguugcauguugucacggccucaaugcaauuuagugugugugauauuuuc

SEQ ID NO: 956 - RNA, 77 nt, Homo sapiens
guggccucguucaaguaauccaggauaggcugugcaggucccaaugggccuauucuugguuacuugcacggggacgc

SEQ ID NO: 957 - RNA, 84 nt, Homo sapiens
ggcuguggcuggauucaaguaauccaggauaggcuguuuccaucugugaggccuauucuugauuacuuguuucuggaggcagcu

SEQ ID NO: 958 - RNA, 77 nt, Homo sapiens
ccgggacccaguucaaguaauucaggauagguugugugcuguccagccuguucuccauuacuuggcucggggaccgg

SEQ ID NO: 959 - RNA, 97 nt, Homo sapiens
accucucuaacaaggugcagagcuuagcugauuggugaacagugauugguuuccgcuuuguucacaguggcuaaguucugcaccugaagagaaggug

SEQ ID NO: 960 - RNA, 86 nt, Homo sapiens
gguccuugcccucaaggagcucacagucuauugaguuaccuuucugacuuucccacuagauugugagcuccuggagggcaggcacu

SEQ ID NO: 961 - RNA, 64 nt, Homo sapiens
augacugauuucuuuugguguucagagucaauauaauuuucuagcaccaucugaaaucgguuau

SEQ ID NO: 962 - RNA, 88 nt, Homo sapiens
aucucuuacacaggcugaccgauuucuccugguguucagagucuguuuuugucuagcaccauuugaaaucgguuaugauguaggggga

SEQ ID NO: 963 - RNA, 88 nt, Homo sapiens
accaaguuucaguucauguaaacauccuacacucagcuguaauacauggauuggcugggagguggauguuuacuucagcugacuugga

SEQ ID NO: 964 - RNA, 89 nt, Homo sapiens
accaugcuguaguguguguaaacauccuacacucucagcugugagcucaagguggcugggagaggguuguuuacuccuucugccaugga

SEQ ID NO: 965 - RNA, 72 nt, Homo sapiens
agauacuguaaacauccuacacucucagcuguggaaaguaagaaagcugggagaaggcuguuuacucuuucu

SEQ ID NO: 966 - RNA, 80 nt, Homo sapiens
cugggggcuccaaagugcuguucgugcagguagugugauuacccaaccuacugcugagcuagcacuucccgagcccccgg

SEQ ID NO: 967 - RNA, 68 nt, Homo sapiens
ccucuacuuuaacauggaggcacuugcugugacaugacaaaaauaagugcuuccauguuugagugugg

SEQ ID NO: 968 - RNA, 81 nt, Homo sapiens
aacacagugggcacucaauaaaugucuguugaauugaaaugcguuacauucaacggguauuuauugagcacccacucugug

SEQ ID NO: 969 - RNA, 119 nt, Homo sapiens
aggauucugcucaugccagggugagguaguaaguuguauuguugugggguagggauauuaggccccaauuagaagauaacuauacaacuuacuacuuucccugguguguggcauauuca

SEQ ID NO: 970 - RNA, 89 nt, Homo sapiens
ugcugcuggccagagcucuuuucacauugugcuacugucugcaccugucacuagcagugcaauguuaaaagggcauuggccguguagug

SEQ ID NO: 971 - RNA, 82 nt, Homo sapiens
ggccugcccgacacucuuucccuguugcacuacuauaggccgcugggaagcagugcaaugaugaaagggcaucggucagguc

SEQ ID NO: 972 - RNA, 86 nt, Homo sapiens
acugcuaacgaaugcucugacuuuauugcacuacuguacuuuacagcuagcagugcaauaguauugucaaagcaucugaaagcagg

SEQ ID NO: 973 - RNA, 101 nt, Homo sapiens
ccgcccccgcgucuccagggcaaccguggcuuucgauuguuacugugggaacuggagguaacagucuacagccauggucgccccgcagcacgcccacgcgc

SEQ ID NO: 974 - RNA, 110 nt, Homo sapiens
cggggcaccccgcccggacagcgcgccggcaccuuggcucuagacugcuuacugcccgggccgcccucaguaacagucuccagucacggccaccgacgccuggccccgcc

SEQ ID NO: 975 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 976 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 977 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 978 - RNA, 73 nt, Homo sapiens
cagggugugugacugguugaccagaggggcaugcacuguguucacccugugggccaccuagucaccaacccuc

SEQ ID NO: 979 - RNA, 102 nt, Homo sapiens
gguccucugacucucuucggugacggguauucuuggguggauaauacggauuacguuguuauugcuuaagaauacgcguagucgaggagaguaccagcggca

SEQ ID NO: 980 - RNA, 87 nt, Homo sapiens
gacagugcagucacccauaaaguagaaagcacuacuaacagcacuggaggguguaguguuuccuacuuuauggaugaguguacugug

SEQ ID NO: 981 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 982 - RNA, 72 nt, Homo sapiens
aaucuaaagacaacauuucugcacacacaccagacuauggaagccaguguguggaaaugcuucugcuagauu

SEQ ID NO: 983 - RNA, 89 nt, Homo sapiens
gccggcgcccgagcucuggcuccgugucuucacucccgugcuuguccgaggagggagggagggacgggggcugugcuggggcagcugga

SEQ ID NO: 984 - RNA, 110 nt, Homo sapiens
cggaaaauuugccaaggguuugggggaacauucaaccugucggugaguuugggcagcucaggcaaaccaucgaccguugaguggacccugaggccuggaauugccauccu

SEQ ID NO: 985 - RNA, 137 nt, Homo sapiens
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 986 - RNA, 110 nt, Homo sapiens
gagcugcuugccuccccccguuuuuggcaaugguagaacucacacuggugagguaacaggauccggugguucuagacuugccaacuauggggcgaggacucagccggcac

SEQ ID NO: 987 - RNA, 110 nt, Homo sapiens
ccgcagagugugacuccuguucuguguauggcacugguagaauucacugugaacagucucagucagugaauuaccgaagggccauaaacagagcagagacagauccacga

SEQ ID NO: 988 - RNA, 84 nt, Homo sapiens
ccagucacguccccuuaucacuuuuccagcccagcuuugugacuguaaguguuggacggagaacugauaaggguaggugauuga

SEQ ID NO: 989 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 990 - RNA, 110 nt, Homo sapiens
aucauucagaaaugguauacaggaaaaugaccuaugaauugacagacaauauagcugaguuugucugucauuucuuuaggccaauauucuguaugacugugcuacuucaa

SEQ ID NO: 991 - RNA, 88 nt, Homo sapiens
cgaggaugggagcugagggcugggucuuugcgggcgagaugagggugucggaucaacuggccuacaaagucccaguucucggcccccg

SEQ ID NO: 992 - RNA, 83 nt, Homo sapiens
guggucucagaaucgggguuuugagggcgagaugaguuuauguuuuauccaacuggcccucaaagucccgcuuuuggggucau

SEQ ID NO: 993 - RNA, 87 nt, Homo sapiens
agcuucccuggcucuagcagcacagaaauauuggcacagggaagcgagucugccaauauuggcugugcugcuccaggcaggguggug

SEQ ID NO: 994 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 995 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 996 - RNA, 70 nt, Homo sapiens
gugaauuagguaguuucauguuguugggccuggguuucugaacacaacaacauuaaaccacccgauucac

SEQ ID NO: 997 - RNA, 110 nt, Homo sapiens
ugcucgcucagcugaucuguggcuuagguaguuucauguuguugggauugaguuuugaacucggcaacaagaaacugccugaguuacaucagucgguuuucgucgagggc

SEQ ID NO: 998 - RNA, 84 nt, Homo sapiens
acuggucggugauuuagguaguuuccuguuguugggauccaccuuucucucgacagcacgacacugccuucauuacuucaguug

SEQ ID NO: 999 - RNA, 75 nt, Homo sapiens
ggcugugccggguagagagggcagugggagguaagagcucuucacccuucaccaccuucuccacccagcauggcc

SEQ ID NO: 1000 - RNA, 71 nt, Homo sapiens
gccaacccaguguucagacuaccuguucaggaggcucucaauguguacaguagucugcacauugguuaggc

SEQ ID NO: 1001 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 1002 - RNA, 90 nt, Homo sapiens
ccgggccccugugagcaucuuaccggacagugcuggauuucccagcuugacucuaacacugucugguaacgauguucaaaggugacccgc

SEQ ID NO: 1003 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 1004 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 1005 - RNA, 110 nt, Homo sapiens
ucaccuggccaugugacuugugggcuucccuuugucauccuucgccuagggcucugagcagggcagggacagcaaaggggugcucaguugucacuucccacagcacggag

SEQ ID NO: 1006 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 1007 - RNA, 71 nt, Homo sapiens
ugacgggcgagcuuuuggcccggguuauaccugaugcucacguauaagacgagcaaaaagcuuguugguca

SEQ ID NO: 1008 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 1009 - RNA, 63 nt, Homo sapiens
aagaaaugguuuaccgucccacauacauuuugaauauguaugugggaugguaaaccgcuucuu

SEQ ID NO: 1010 - RNA, 69 nt, Homo sapiens
ccaccacuuaaacguggauguacuugcuuugaaacuaaagaaguaagugcuuccauguuuuggugaugg

SEQ ID NO: 1011 - RNA, 73 nt, Homo sapiens
gcucccuucaacuuuaacauggaagugcuuucugugacuuuaaaaguaagugcuuccauguuuuaguaggagu

SEQ ID NO: 1012 - RNA, 83 nt, Homo sapiens
cugacuaugccuccccgcauccccuagggcauugguguaaagcuggagacccacugccccaggugcugcuggggguuguaguc

SEQ ID NO: 1013 - RNA, 80 nt, Homo sapiens
gguaccugaagagagguuuucuggguuucuguuucuuuaaugaggacgaaacacaccugguuaaccucuuuuccaguauc

SEQ ID NO: 1014 - RNA, 84 nt, Homo sapiens
gugguaccugaagagagguuuucuggguuucuguuucuuuauugaggacgaaacacaccugguuaaccucuuuuccaguaucaa

SEQ ID NO: 1015 - RNA, 94 nt, Homo sapiens
gaguuugguuuuguuuggguuuguucuagguauggucccagggaucccagaucaaaccaggccccugggccuauccuagaaccaaccuaagcuc

SEQ ID NO: 1016 - RNA, 93 nt, Homo sapiens
guagucaguaguuggggggugggaacggcuucauacaggaguugaugcacaguuauccagcuccuauaugaugccuuucuucauccccuucaa

SEQ ID NO: 1017 - RNA, 95 nt, Homo sapiens
uuguaccuggugugauuauaaagcaaugagacugauugucauaugucguuugugggauccgucucaguuacuuuauagccauaccugguaucuua

SEQ ID NO: 1018 - RNA, 99 nt, Homo sapiens
gaaacugggcucaaggugaggggugcuaucugugauugagggacaugguuaauggaauugucucacacagaaaucgcacccgucaccuuggccuacuua

SEQ ID NO: 1019 - RNA, 86 nt, Homo sapiens
gucugucugcccgcaugccugccucucuguugcucugaaggaggcaggggcugggccugcagcugccugggcagagcggcuccugc

SEQ ID NO: 1020 - RNA, 65 nt, Homo sapiens
cuugaauccuuggaaccuaggugugagugcuauuucagugcaacacaccuauucaaggauucaaa

SEQ ID NO: 1021 - RNA, 66 nt, Homo sapiens
aaaagguggauauuccuucuauguuuauguuauuuaugguuaaacauagaggaaauuccacguuuu

SEQ ID NO: 1022 - RNA, 67 nt, Homo sapiens
guggcacucaaacugugggggcacuuucugcucucuggugaaagugccgccaucuuuugaguguuac

SEQ ID NO: 1023 - RNA, 68 nt, Homo sapiens
uaaaagguagauucuccuucuaugaguacauuauuuaugauuaaucauagaggaaaauccacguuuuc

SEQ ID NO: 1024 - RNA, 80 nt, Homo sapiens
gguauuuaaaagguagauuuuccuucuaugguuacguguuugaugguuaaucauagaggaaaauccacguuuucaguauc

SEQ ID NO: 1025 - RNA, 100 nt, Homo sapiens
caguccuucuuugguauuuaaaacguggauauuccuucuauguuuacgugauuccugguuaaucauagaggaaaauccauguuuucaguaucaaaugcug

SEQ ID NO: 1026 - RNA, 69 nt, Homo sapiens
uugagcagagguugcccuuggugaauucgcuuuauuuauguugaaucacacaaaggcaacuuuuguuug

SEQ ID NO: 1027 - RNA, 66 nt, Homo sapiens
agggcuccugacuccagguccuguguguuaccuagaaauagcacuggacuuggagucagaaggccu

SEQ ID NO: 1028 - RNA, 61 nt, Homo sapiens
aagaugguugaccauagaacaugcgcuaucucugugucguauguaauaugguccacaucuu

SEQ ID NO: 1029 - RNA, 76 nt, Homo sapiens
uacuugaagagaaguuguucgugguggauucgcuuuacuuaugacgaaucauucacggacaacacuuuuuucagua

SEQ ID NO: 1030 - RNA, 91 nt, Homo sapiens
cugggguacggggauggauggucgaccaguuggaaaguaauuguuucuaauguacuucaccugguccacuagccguccguauccgcugcag

SEQ ID NO: 1031 - RNA, 90 nt, Homo sapiens
gagagaagcacuggacuuagggucagaaggccugagucucucugcugcagaugggcucucugucccugagccaagcuuuguccucccugg

SEQ ID NO: 1032 - RNA, 94 nt, Homo sapiens
auaaaggaaguuaggcugaggggcagagagcgagacuuuucuauuuuccaaaagcucggucugaggccccucagucuugcuuccuaacccgcgc

SEQ ID NO: 1033 - RNA, 114 nt, Homo sapiens
uccugcuuguccugcgaggugucuugcaggccgucaugcaggccacacugacgguaacguugcaggucgucuugcagggcuucucgcaagacgacauccucaucaccaacgacg

SEQ ID NO: 1034 - RNA, 94 nt, Homo sapiens
ugacuccuccaggucuuggaguaggucauuggguggauccucuauuuccuuacgugggccacuggauggcuccuccaugucuuggaguagauca

SEQ ID NO: 1035 - RNA, 93 nt, Homo sapiens
ccggggagaaguacggugagccugucauuauucagagaggcuagauccucuguguugagaaggaucaugaugggcuccucgguguucuccagg

SEQ ID NO: 1036 - RNA, 91 nt, Homo sapiens
cugugugugaugagcuggcaguguauuguuagcugguugaauaugugaauggcaucggcuaacaugcaacugcugucuuauugcauauaca

SEQ ID NO: 1037 - RNA, 72 nt, Homo sapiens
cuugggaauggcaaggaaaccguuaccauuacugaguuuaguaaugguaaugguucucuugcuauacccaga

SEQ ID NO: 1038 - RNA, 85 nt, Homo sapiens
gcuaagcacuuacaacuguuugcagaggaaacugagacuuuguaacuaugucucagucucaucugcaaagaaguaagugcuuugc

SEQ ID NO: 1039 - RNA, 96 nt, Homo sapiens
ucccuggcgugaggguaugugccuuuggacuacaucguggaagccagcaccaugcaguccaugggcauauacacuugccucaaggccuaugucauc

SEQ ID NO: 1040 - RNA, 76 nt, Homo sapiens
gagggggaagacgggaggaaagaagggagugguuccaucacgccuccucacuccucuccucccgucuucuccucuc

SEQ ID NO: 1041 - RNA, 73 nt, Homo sapiens
acuuggagagaggcuggccgugaugaauucgauucaucaaagcgagucauacacggcucuccucucuuuuagu

SEQ ID NO: 1042 - RNA, 83 nt, Homo sapiens
gagaaucaucucucccagauaauggcacucucaaacaaguuuccaaauuguuugaaaggcuauuucuuggucagaugacucuc

SEQ ID NO: 1043 - RNA, 84 nt, Homo sapiens
guggcagcuugguggucguaugugugacgccauuuacuugaaccuuuaggagugacaucacauauacggcagcuaaacugcuac

SEQ ID NO: 1044 - RNA, 84 nt, Homo sapiens
uugacuuagcuggguaguggggaacccuuccaugaggaguagaacacuccuuaugcaagauucccuucuaccuggcuggguugg

SEQ ID NO: 1045 - RNA, 89 nt, Homo sapiens
cuggccuccagggcuuuguacaugguaggcuuucauucauucguuugcacauucggugaaggucuacugugugccaggcccugugccag

SEQ ID NO: 1046 - RNA, 81 nt, Homo sapiens
gauacucgaaggagagguuguccguguugucuucucuuuauuuaugaugaaacauacacgggaaaccucuuuuuuaguauc

SEQ ID NO: 1047 - RNA, 84 nt, Homo sapiens
gcucuuccucucuaauccuuugucccugggugagagugcuuucugaaugcaaugcacccgggcaaggauucugagagggugagc

SEQ ID NO: 1048 - RNA, 83 nt, Homo sapiens
gcugcuguugggagacccuggucugcacucuaucuguauucuuacugaagggagugcagggcaggguuucccauacagagggc

SEQ ID NO: 1049 - RNA, 84 nt, Homo sapiens
gaugcacccagugggggagccaggaaguauugauguuucugccaguuuagcgucaacacuugcugguuuccucucuggagcauc

SEQ ID NO: 1050 - RNA, 124 nt, Homo sapiens
gccaccaccaucagccauacuauguguagugccuuauucaggaagguguuacuuaauagauuaauauuuguaaggcacccuucugaguagaguaaugugcaacauggacaacauuugugguggc

SEQ ID NO: 1051 - RNA, 94 nt, Homo sapiens
gugcuguguguagugcuucacuucaagaagugccaugcaugugucuagaaauauguuuugcaccuuuuggagugaaauaaugcacaacagauac

SEQ ID NO: 1052 - RNA, 94 nt, Homo sapiens
caugcugugugugguacccuacugcagacaguggcaaucauguauaauuaaaaaugauugguacgucuguggguagaguacugcaugacacaug

SEQ ID NO: 1053 - RNA, 74 nt, Homo sapiens
gugguguccuacucaggagaguggcaaucacauguaauuaggugugauugaaaccucuaagaguggaguaacac

SEQ ID NO: 1054 - RNA, 87 nt, Homo sapiens
caauagacacccaucgugucuuuugcucugcagucaguaaauauuuuuuugugaauguguagcaaaagacagaauggugguccauug

SEQ ID NO: 1055 - RNA, 87 nt, Homo sapiens; identical to SEQ ID NO: 1054

SEQ ID NO: 1056 - RNA, 84 nt, Homo sapiens
ucucagucuguggcacucagccuugagggcacuuucuggugccagaaugaaagugcugucauagcugagguccaaugacugagg

SEQ ID NO: 1057 - RNA, 98 nt, Homo sapiens
gguacuucucagucuguggcacucagccuugagggcacuuucuggugccagaaugaaagugcugucauagcugagguccaaugacugaggcgagcacc

SEQ ID NO: 1058 - RNA, 129 nt, Homo sapiens
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 1059 - RNA, 127 nt, Homo sapiens
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 1060 - RNA, 90 nt, Homo sapiens
ucucaggcugugaccuucucgaggaaagaagcacuuucuguugucugaaagaaaagaaagugcuuccuuucagaggguuacgguuugaga

SEQ ID NO: 1061 - RNA, 90 nt, Homo sapiens
ucucagguugugaccuucucgaggaaagaagcacuuucuguugucugaaagaaaagaaagugcuuccuuucagaggguuacgguuugaga

SEQ ID NO: 1062 - RNA, 85 nt, Homo sapiens
ucucaugaugugaccaucuggagguaagaagcacuuuguguuuugugaaagaaagugcuuccuuucagaggguuacucuuugaga

SEQ ID NO: 1063 - RNA, 90 nt, Homo sapiens
ucucaggcugugaccaucuggagguaagaagcacuuucuguuuugugaaagaaaagaaagugcuuccuuucagaggguuacucuuugaga

SEQ ID NO: 1064 - RNA, 87 nt, Homo sapiens
ucucaggcagugacccucuagauggaagcacugucuguuguauaaaagaaaagaucgugcaucccuuuagaguguuacuguuugaga

SEQ ID NO: 1065 - RNA, 67 nt, Homo sapiens
gugacccucuagauggaagcacugucuguugucuaagaaaagaucgugcaucccuuuagaguguuac

SEQ ID NO: 1066 - RNA, 95 nt, Homo sapiens
gaagaucucaggcagugacccucuagauggaagcacugucuguugucuaagaaaagaucgugcauccuuuuagaguguuacuguuugagaaaauc

SEQ ID NO: 1067 - RNA, 83 nt, Homo sapiens
ucaugcuguggcccuccagagggaagcgcuuucuguugucugaaagaaaacaaagcgcuccccuuuagagguuuacgguuuga

SEQ ID NO: 1068 - RNA, 101 nt, Homo sapiens
gcgagaagaucucaugcugugacucucuggagggaagcacuuucuguugucugaaagaaaacaaagcgcuucucuuuagaguguuacgguuugagaaaagc

SEQ ID NO: 1069 - RNA, 87 nt, Homo sapiens
ucccaugcugugacccucuagagggaagcacuuucuguugucugaaagaaaccaaagcgcuucccuuuggagcguuacgguuugaga

SEQ ID NO: 1070 - RNA, 88 nt, Homo sapiens
ucucaggcugugacccucuagagggaagcgcuuucuguuggcuaaaagaaaagaaagcgcuucccuucagaguguuaacgcuuugaga

SEQ ID NO: 1071 - RNA, 87 nt, Homo sapiens
ucucaugcugugacccucuagagggaagcacuuucucuugucuaaaagaaaagaaagcgcuucucuuuagaggauuacucuuugaga

SEQ ID NO: 1072 - RNA, 88 nt, Homo sapiens
ucccaugcugugacccucuagaggaagcacuuucuguuuguugucugagaaaaaacaaagugcuucccuuuagaguuacuguuuggga

SEQ ID NO: 1073 - RNA, 87 nt, Homo sapiens
ucucaugcugugacccucuagagggaagcgcuuucuguugucugaaagaaaagaacgcgcuucccuauagaggguuacccuuugaga

SEQ ID NO: 1074 - RNA, 87 nt, Homo sapiens
ucucaugcugugacccuacaaagggaagcacuuucucuuguccaaaggaaaagaaggcgcuucccuuuggaguguuacgguuugaga

SEQ ID NO: 1075 - RNA, 85 nt, Homo sapiens
cucaagcugugacucuccagagggaugcacuuucucuuaugugaaaaaaaagaaggcgcuucccuuuagagcguuacgguuuggg

SEQ ID NO: 1076 - RNA, 85 nt, Homo sapiens
cucaggcugugacccucuagagggaagcacuuucuguugcuugaaagaagagaaagcgcuuccuuuuagaggauuacucuuugag

SEQ ID NO: 1077 - RNA, 65 nt, Homo sapiens
gugacccucuagagggaagcacuuucuguugaaagaaaagaacaugcauccuuucagaggguuac

SEQ ID NO: 1078 - RNA, 85 nt, Homo sapiens
ucucaagcugugacugcaaagggaagcccuuucuguugucuaaaagaaaagaaagugcuucccuuuggugaauuacgguuugaga

SEQ ID NO: 1079 - RNA, 77 nt, Mus musculus
gcuugggacacauacuucuuuauaugcccauaugaaccugcuaagcuauggaauguaaagaaguauguauuucaggc

SEQ ID NO: 1080 - RNA, 72 nt, Mus musculus
ucagagcacauacuucuuuauguacccauaugaacauucagugcuauggaauguaaagaaguauguauuuug

SEQ ID NO: 1081 - RNA, 87 nt, Rattus norvegicus
ugccuacucagagcacauacuucuuuauguacccauaugaacauagaaugcuauggaauguaaagaaguguguauuuuggguaggua

SEQ ID NO: 1082 - RNA, 85 nt, Pan paniscus; identical to SEQ ID NO: 941

SEQ ID NO: 1083 - RNA, 86 nt, Monodelphis domestica
accuacucagaguacauacuucuuuauguacccauaugaacauacaaugcuauggaauguaaagaaguauguauuuuugguaggug

SEQ ID NO: 1084 - RNA, 71 nt, Gallus gallus
ugagagacauacuucuuuauaugcccauaugaaccuggcaaucuauggaauguaaagaaguauguauuuca

SEQ ID NO: 1085 - RNA, 85 nt, Gallus gallus
accugcucagagcacauacuucuuuauguacccauaugaacauacaaugcuauggaauguaaagaaguauguauuuuuggcaggc

SEQ ID NO: 1086 - RNA, 64 nt, Gallus gallus
acauacuucuucauaugcccauauggagucggccggcguuauggaauguuaagaaguauguauc

SEQ ID NO: 1087 - RNA, 62 nt, Xenopus tropicalis
gcacauacuucuuuauaugcccauacugaaugaaaaugcuauggaauguuaagaaguaugua

SEQ ID NO: 1088 - RNA, 106 nt, Danio rerio
gcccauauaucccgcuugguagacauacuucuuuauaugcccauaugaacaagagcagcuauggaauguaaagaaguauguaucccaggugagagaaaaacggggc

SEQ ID NO: 1089 - RNA, 87 nt, Danio rerio
gccuacuugguguacauacuucuuuaugugcccauaugaacauauaaaagcuauggaauguaaagaaguauguauucuuggucaggu

SEQ ID NO: 1090 - RNA, 76 nt, Fugu rubripes
cuuaguguacauacuucuuuauguacccauauggacauaugauaacuauggaauguaaagaaguauguauucuugg

SEQ ID NO: 1091 - RNA, 92 nt, Tetraodon nigroviridis
auuaccuccuuaguguacauacuucuuuauguacccauaugaacauaugauaacuauggaauguaaagaaguauguauucuuggugagguga

SEQ ID NO: 1092 - RNA, 73 nt, Mus musculus
ccaggccacaugcuucuuuauauccucauagauaucucagcacuauggaauguaaggaagugugugguuuugg

SEQ ID NO: 1093 - RNA, 84 nt, Rattus norvegicus
cuuccccaggccacaugcuucuuuauauccucauagauaucacugcgcuauggaauguaaggaagugugugguuuuggcaagug

SEQ ID NO: 1094 - RNA, 86 nt, Macaca nemestrina; identical to SEQ ID NO: 942

SEQ ID NO: 1095 - RNA, 86 nt, Pongo pygmaeus; identical to SEQ ID NO: 942

SEQ ID NO: 1096 - RNA, 76 nt, Monodelphis domestica
gaggcaacaugcuucuuuauauccccauaugaauuaugcugcuauggaauguaaggaagugugugguuucgggaag

SEQ ID NO: 1097 - RNA, 76 nt, Gallus gallus
gagaugacaugcuucuuuauauccccauauggauuaggcugcuauggaauguaaggaagugugugguuucagggag

SEQ ID NO: 1098 - RNA, 70 nt, Xenopus tropicalis
gaggucacaugcuucuuuauauacccauaugaacuacauuguuauggaauguaaggaaguguguggcuuc

SEQ ID NO: 1099 - RNA, 99 nt, Danio rerio
gaauguugccucuugugaagacaugcuuccuuauaugcccauauuaaugcucaaguuauggaauguaaggaagugugugguuucagggggaaauuuguc

SEQ ID NO: 1100 - RNA, 109 nt, Danio rerio
gaguuggcuguugcuuauugagacgacauacuuccuuauauccccauauucagaauuaaucuauggaauguaaggaaguguguggcuucagugagaucucaucaucauc

SEQ ID NO: 1101 - RNA, 111 nt, Mus musculus
aggagcggaguacgugagccagugcuauguggaagacuugugauuuuguuguucugauaugauaugacaacaagucacagccagccucauagcguggacuccuaucaccuu

SEQ ID NO: 1102 - RNA, 97 nt, Rattus norvegicus
uguuggccuaguucuguguggaagacuagugauuuuguuguuuuuagauaacuaagacgacaacaaaucacagucugccauauggcacaggccaccu

SEQ ID NO: 1103 - RNA, 95 nt, Rattus norvegicus
ggacagaccagcccugucuggaagacuagugauuuuguuguugugucuguguccaacaacaagucccagucugccacaugguguuggucacauca

SEQ ID NO: 1104 - RNA, 110 nt, Rattus norvegicus
aggccagaacacaugagccaaugcuauguggaagacuugugauuuuguuguucugauaugauaugacaacaagucacagccagccucauagaguggacucccaucaccuu

SEQ ID NO: 1105 - RNA, 110 nt, Lagothrix lagotricha; identical to SEQ ID NO: 943

SEQ ID NO: 1106 - RNA, 110 nt, Saguinus labiatus
uuggauauuggccuaguucuguguggaagacuagugauuuuguuguuuuuagauaauuaaaucgacaacaaaucacagucugccauauggcacaggccaugccucuacag

SEQ ID NO: 1107 - RNA, 110 nt, Macaca nemestrina
uuggauguuggccuaguucuguguggaagacuagugauuuuguuguuuuuagauaacuaaauugacaacaaaucacagucugccauauggcacaggccaugccucuacag

SEQ ID NO: 1108 - RNA, 110 nt, Macaca nemestrina
cuggauacagagugaaguggcuggccccgucuggaagacuagugauuuuguuguugucuuacugcgcucaacaacgaaucccagucugccgaauggugccagccauugca

SEQ ID NO: 1109 - RNA, 110 nt, Gorilla gorilla; identical to SEQ ID NO: 943

SEQ ID NO: 1110 - RNA, 110 nt, Gorilla gorilla
cugcauacagaguggaccggcuggccccaucuggaagacuagugauuuuguuguugucuuacugcgcucaacaacaaaucccagucugccuaauggugccagccaucgca

SEQ ID NO: 1111 - RNA, 110 nt, Gorilla gorilla
agacuggaguggcuguggucuagugcuguguggaagacuagugauuuuguuguucugauguacuacgacaacaagucacagccggccucacagcgcagacucccuucgac

SEQ ID NO: 1112 - RNA, 110 nt, Pan paniscus; identical to SEQ ID NO: 943

SEQ ID NO: 1113 - RNA, 110 nt, Pan paniscus
cuggauacagagcggaccagcuggccccaucuggaagacuagugauuuuguuguugucuuacugcgcucaacaacaaaucccagucuaccuaauggugccagccaucaca

SEQ ID NO: 1114 - RNA, 110 nt, Pan paniscus; identical to SEQ ID NO: 945

SEQ ID NO: 1115 - RNA, 110 nt, Pan troglodytes; identical to SEQ ID NO: 945

SEQ ID NO: 1116 - RNA, 110 nt, Pongo pygmaeus; identical to SEQ ID NO: 943

SEQ ID NO: 1117 - RNA, 110 nt, Pongo pygmaeus
cuggauacagaguggaccggcuggccccgucuggaagacuagugauuuuguuguugucuuacugcgcucaacaacaaaucccagucugccuaauggugccagccaucgca

SEQ ID NO: 1118 - RNA, 77 nt, Monodelphis domestica
uuggccuaguucuguguggaagacuagugauuuuguuguuuuuagauaacuaaaacgacaacaaaucacagucugcc

SEQ ID NO: 1119 - RNA, 109 nt, Gallus gallus
uggauguuggucuaguucuguguggaagacuagugauuuuguuguuuuuagauaauuaaauugacaacaaaucacagucugccauauggcacagaucaugccucuacag

SEQ ID NO: 1120 - RNA, 99 nt, Gallus gallus
ggacggccgcggugcccucuggaagacuagugauuuuguuguuguauggcucaucccaccacaacaagucacagucugccuuagggcgcacggccccgc

SEQ ID NO: 1121 - RNA, 95 nt, Gallus gallus
cuguggucuggcucuguguggaagacuagugauuuuguuguuaugauuuauaaaggugacaacaaaucauagccugccauacagcacagaucuuc

SEQ ID NO: 1122 - RNA, 110 nt, Gallus gallus
uggacauuggucuaguucuguguggaagacuagugauuuuuguuguuuuuagauaacuaaauugacaacaaaucacagucuaccauauggcacagacuaugccucuacag

SEQ ID NO: 1123 - RNA, 108 nt, Xenopus tropicalis
uggauguugguuuaguucuguguggaagacuagugauuuuguuguuuuuagauaacaacauugacaacaaaucgcagucugccauauggcacagaccaugccucuaca

SEQ ID NO: 1124 - RNA, 103 nt, Xenopus tropicalis
gaggugcaggcugacucuuuguggaagacuagugauuuuguuguuguaagccuuauugcaugacaacaagucacagucugccucacagugcccagcaauauca

SEQ ID NO: 1125 - RNA, 88 nt, Xenopus tropicalis
cugggucgguuuggaagacuagugauuuuguuguuuuuauaaaaguuugucacaaacagcaaaucguagucuccacucugccccagga

SEQ ID NO: 1126 - RNA, 82 nt, Bos taurus
agacuggagcgguuguggucuagugcuguguggaagacuagugauuuuguuguucugauguacuaugacaacaagucacagc

SEQ ID NO: 1127 - RNA, 109 nt, Sus scrofa
uggauguuggucuaguucuguguggaagacuagugauuuuguuguuuuuagauaacuaaaucgacaacaaaucacagucugccauauggcacaggccaugccucuacag

SEQ ID NO: 1128 - RNA, 79 nt, Danio rerio
uguggaagacuagugauuuuguuguuguuucuucugcuuucugacaacaagucacagucuaccucagcgagcgggcccu

SEQ ID NO: 1129 - RNA, 97 nt, Danio rerio
uuuugggcauuauggaagacuagugauuuuguuguugugacgucaucaguacugacaacaagucacagucugccucagugaccagaauccacucguc

SEQ ID NO: 1130 - RNA, 120 nt, Danio rerio
uguguguguguucugcccucugcggaguggaagacuagugauuuuguugugaaaaugaacaaaaaccaacaacaaaccgcagucguccucucagcacggggcccacaccugcagagcaca

SEQ ID NO: 1131 - RNA, 104 nt, Danio rerio
ugaacgcuggcuugcuucuguguggaagacuugugauuuuguuguuguuaguuagaugaagugacaacaaaucacggucugcccuacagcacaggcccagcauc

SEQ ID NO: 1132 - RNA, 91 nt, Fugu rubripes
cccugucugcugcuguguggaagacuagugauuuuguuguuuuuaguuagaucaacugacaacaaaucacagucugccaaacagcacaggc

SEQ ID NO: 1133 - RNA, 65 nt, Tetraodon nigroviridis
uggaagacuagugauuuuguuguuuggugucaaaauugacaacaaaucauugucuuccucacugc

SEQ ID NO: 1134 - RNA, 110 nt, Mus musculus
gaccugucugucuucuguauauacccuguagauccgaauuuguguaaggaauuuuguggucacaaauucguaucuaggggaauauguaguugacauaaacacuccgcuca

SEQ ID NO: 1135 - RNA, 110 nt, Ateles geoffroyi
gaucugucugucuucuguauauacccuguagauccgaauuuguguaaggaauuuuguggucacaaauucguaucuaggggaauauguaguugacauaaacacuccgcuca

SEQ ID NO: 1136 - RNA, 110 nt, Saguinus labiatus; identical to SEQ ID NO: 1135

SEQ ID NO: 1137 - RNA, 109 nt, Macaca nemestrina
cagagguuguaacguugucuauauauacccuguagaaccgaauuugugugguauccauauagucacagauucgauucuaggggaauauauggucgaugcaaaaacuuca

SEQ ID NO: 1138 - RNA, 110 nt, Gorilla gorilla; identical to SEQ ID NO: 946

SEQ ID NO: 1139 - RNA, 110 nt, Gorilla gorilla
ccagacauuguaacguugucuauauauacccuguagaaccgaauuugugugguauccauauagucacagauucgauucuaggggaauauauggucgaugcaaaaacuuca

SEQ ID NO: 1140 - RNA, 110 nt, Pan paniscus
gaucugucugucuucuguauauacccuguagauccgaauuuguguaaggaauuuuguggucacacauucguaucuaggggaauauguaguugacauacacacuccgcucu

SEQ ID NO: 1141 - RNA, 110 nt, Pan paniscus
ccagagguuguaacguugucuauauauacccuguagaaccgaauuugugugguauccguauagucacagauucgauucuaggggaauauauggucgaugcaaaaacuuca

SEQ ID NO: 1142 - RNA, 90 nt, Monodelphis domestica
cugucuucuguauauacccuguagauccgaauuuguguaaggaauuuuguggucacaaauucguaucuaggggaauauguaguugacaua

SEQ ID NO: 1143 - RNA, 110 nt, Gallus gallus
cagaacguuauuacguugucuauauauacccuguagaaccgaauuugugugauauucauauagucacagauucgauucuaggggaauauauggucgaugcaaaaacuuca

SEQ ID NO: 1144 - RNA, 100 nt, Xenopus tropicalis
gauuugccuguccucuguauguacccuguagauccgaauuugugugagcgcaaucauaucacaaauucgugucuggggggauaugcaguugacacaaacg

SEQ ID NO: 1145 - RNA, 88 nt, Xenopus tropicalis
aacguugucuauauguacccuguagaaccgaauuugugugguucguacagucacagauucgauucuagggggauauauggucgaugca

SEQ ID NO: 1146 - RNA, 71 nt, Xenopus tropicalis
uauaugcacccuguagaaucgaauuugugugaguucugaaccacagauucgucucuagggggguauauggg

SEQ ID NO: 1147 - RNA, 109 nt, Bos taurus
gaucugucugucuucuguauauacccuguagauccgaauuuguguaaggaauuuugugaucacaaauucguaucuaggggaauauguaguugacauaaacacuccgcuc

SEQ ID NO: 1148 - RNA, 99 nt, Bos taurus
cagugacguugucuauauauacccuguagaaccgaauuugugugguauccauguagucacagauucgauucuaggggaauauauggucgaugcaaaaac

SEQ ID NO: 1149 - RNA, 99 nt, Danio rerio
ugucugucaucuauauauacccuguagauccgaauuugugugaauauacagucgcaaauucgugucuuggggaauauguaguugacauaaacacaacgc

SEQ ID NO: 1150 - RNA, 78 nt, Danio rerio
gucuauauauacccuguagaaccgaauuugugugaaaaaauaacauucacagauucgauucuaggggaguauaugguc

SEQ ID NO: 1151 - RNA, 92 nt, Danio rerio
guagucgucuauauguacccuguagaaccgaauuuguguccaaaacaucaaaaucgcaaauacgucucuacaggaauacaugggcgacguaa

SEQ ID NO: 1152 - RNA, 95 nt, Danio rerio
ccugucaucuauauauacccuguagauccggauuuguguaaacagacgcacagucacaaauucguaucuaggggaguauguaguugauguauagg

SEQ ID NO: 1153 - RNA, 118 nt, Danio rerio
uggaagcuuuguuccgucgucuauauauacccuguagaaccgaauguguguuuacacagcaaauucacagauucgguuuuaggggaguauauggacgaugcaaaaacgucugcuuuca

SEQ ID NO: 1154 - RNA, 118 nt, Danio rerio; identical to SEQ ID NO: 1153

SEQ ID NO: 1155 - RNA, 70 nt, Fugu rubripes
auauauacccuguagaaccgaauuugugugauggcgucaaagucacagauucgauucuaggggaguauau

SEQ ID NO: 1156 - RNA, 84 nt, Fugu rubripes
guugucuauauguacccuguagaaccgaauuugugugaguuccagacagucgcaaguacgucucuacaggaauacaugggcaac

SEQ ID NO: 1157 - RNA, 102 nt, Fugu rubripes
cugucuucuauaucuacccuguagauccggauuuguguaaaaaucauuaaagcaaucacaaauucgcuucuaggggaguauauaguggauuuauacacgacg

SEQ ID NO: 1158 - RNA, 112 nt, Fugu rubripes
ccggugagguggaucgucgucuauaaauacccuguagaaccgaaugugugugcagcugacuugaucacagauuggguucuaggggagucuaugggcgaugaauaaucacuga

SEQ ID NO: 1159 - RNA, 83 nt, Tetraodon nigroviridis
guugucuauauguacccuguagaaccgaauuugugugaguucagacagucacaaguacgucucuacaggaauacaugggcaac

SEQ ID NO: 1160 - RNA, 70 nt, Tetraodon nigroviridis
auauauacccuguagaaccgaauuugugugaucaagucacagucacagauucgauucuaggggaguauau

SEQ ID NO: 1161 - RNA, 108 nt, Tetraodon nigroviridis
gagccgcugucuucuauaucuacccuguagauccggauuuguguaacgaucauuaaagcaaucacaaauucgcuucuaggggaguauauaguggauuuauacacgacg

SEQ ID NO: 1162 - RNA, 120 nt, Tetraodon nigroviridis
gccggugaggugcucgucgucuauacauacccuguagaaccgaaugugugugcagcugacuugaucacagauuggguucuaggggagucuaugggcgcugaauaaucaucgaugaacggc

SEQ ID NO: 1163 - RNA, 96 nt, Mus musculus
ugcgugcuuuuuguucuaaggugcaucuagugcagauagugaaguagacuagcaucuacugcccuaagugcuccuucuggcauaagaaguuauguc

SEQ ID NO: 1164 - RNA, 96 nt, Rattus norvegicus; identical to SEQ ID NO: 1163

SEQ ID NO: 1165 - RNA, 71 nt, Ateles geoffroyi; identical to SEQ ID NO: 947

SEQ ID NO: 1166 - RNA, 71 nt, Lagothrix lagotricha; identical to SEQ ID NO: 947

SEQ ID NO: 1167 - RNA, 71 nt, Saguinus labiatus; identical to SEQ ID NO: 947

SEQ ID NO: 1168 - RNA, 71 nt, Macaca mulatta; identical to SEQ ID NO: 947

SEQ ID NO: 1169 - RNA, 71 nt, Macaca nemestrina; identical to SEQ ID NO: 947

SEQ ID NO: 1170 - RNA, 71 nt, Gorilla gorilla; identical to SEQ ID NO: 947

SEQ ID NO: 1171 - RNA, 71 nt, Pan paniscus; identical to SEQ ID NO: 947

SEQ ID NO: 1172 - RNA, 71 nt, Pan troglodytes; identical to SEQ ID NO: 947

SEQ ID NO: 1173 - RNA, 71 nt, Pongo pygmaeus; identical to SEQ ID NO: 947

SEQ ID NO: 1174 - RNA, 71 nt, Lemur catta; identical to SEQ ID NO: 947

SEQ ID NO: 1175 - RNA, 72 nt, Monodelphis domestica
uuguucuaaggugcaucuagugcagauagugaaguagauuagcaucuacugcccuaagugcuccuucuggca

SEQ ID NO: 1176 - RNA, 93 nt, Gallus gallus
gugcuuuuuguacuaaggugcaucuagugcagauagugaaguagauuagcaucuacugcccuaagugcuccuucuggcauaagaaguuauguc

SEQ ID NO: 1177 - RNA, 87 nt, Xenopus tropicalis
gugcuuuuuguccuaaggugcaucuagugcagauagugaaguagauuagcaucuacugcccuaagugcuccuucuggcauaaaaagu

SEQ ID NO: 1178 - RNA, 71 nt, Bos taurus; identical to SEQ ID NO: 947

SEQ ID NO: 1179 - RNA, 92 nt, Sus scrofa
gugcuuuuuguucuaaggugcaucuagugcagauagugaaguagauuagcaucuacugcccuaagugcuccuucuggcauaagaaguuaugu

SEQ ID NO: 1180 - RNA, 83 nt, Danio rerio
ggcuuugugcuaaggugcaucuagugcagauagugaaguagacuagcaccuacugcccuaagugcuccuucuggcacgagggu

SEQ ID NO: 1181 - RNA, 85 nt, Danio rerio
gccuuccugcuaaggugcaucuuguguaguuagugaaguagucuaguaucuacugcgcuagauguuccuuuuggcaggaguagcu

SEQ ID NO: 1182 - RNA, 76 nt, Fugu rubripes
uugugcuaaggugcaucuagugcagauagugaaauagacuagcaccuacugcccuaagugcuccuucuggcauaag

SEQ ID NO: 1183 - RNA, 76 nt, Tetraodon nigroviridis; identical to SEQ ID NO: 1182

SEQ ID NO: 1184 - RNA, 83 nt, Mus musculus
ucucuuguguuaaggugcaucuagugcuguuagugaagcagcuuacaaucuacugcccuaaaugccccuucucgcacaggcua

SEQ ID NO: 1185 - RNA, 84 nt, Gallus gallus
cgugguccuuguguuaaggugcaucuagugcaguuagugacguagcguagaaucuacugcccuaaaugcuccuucuggcacaag

SEQ ID NO: 1186 - RNA, 78 nt, Xenopus tropicalis
ccuuguguuaaggugcaucuagugcaguuagugacauaguguagcaucuacugcccuaaaugcuccuuuuggcacagg

SEQ ID NO: 1187 - RNA, 75 nt, Bos taurus
cuuguguuaaggugcaucuagugcaguuagugaagcagcucagaaucuacugcccuaaaugcuccuucuggcaca

SEQ ID NO: 1188 - RNA, 87 nt, Danio rerio
uacugcuuaugcuaaggugcauuuagugcagauagugaaauagacuagcaccuacugcccuaagugccccuucuggcauaaggggcu

SEQ ID NO: 1189 - RNA, 80 nt, Mus musculus
ccuaguagugccaaagugcucauagugcagguaguuuuuauaccacucuacugcagugugagcacuucuaguacuccugg

SEQ ID NO: 1190 - RNA, 71 nt, Ateles geoffroyi
guagcacuaaagugcuuauagugcagguaguguuuaguuaucuacugcauuaugagcacuuaaaguacugc

SEQ ID NO: 1191 - RNA, 71 nt, Lagothrix lagotricha; identical to SEQ ID NO: 1190

SEQ ID NO: 1192 - RNA, 71 nt, Saguinus labiatus; identical to SEQ ID NO: 1190

SEQ ID NO: 1193 - RNA, 71 nt, Macaca mulatta; identical to SEQ ID NO: 1190

SEQ ID NO: 1194 - RNA, 71 nt, Macaca nemestrina; identical to SEQ ID NO: 1190

SEQ ID NO: 1195 - RNA, 71 nt, Gorilla gorilla; identical to SEQ ID NO: 1190

SEQ ID NO: 1196 - RNA, 71 nt, Pan paniscus; identical to SEQ ID NO: 1190

SEQ ID NO: 1197 - RNA, 71 nt, Pan troglodytes; identical to SEQ ID NO: 1190

SEQ ID NO: 1198 - RNA, 71 nt, Pongo pygmaeus; identical to SEQ ID NO: 1190

SEQ ID NO: 1199 - RNA, 71 nt, Lemur catta
guagcacuaaagugcuuauagugcagguaguguuuaguuaucuacugcaucaugagcacuuaaaguacugc

SEQ ID NO: 1200 - RNA, 102 nt, Monodelphis domestica
ugaaaggacagcuuuuguagcacuaaagugcuuauagugcagguaguguuuaguuaucuacugcauuaugagcacuugaaguacugcuagcuguagaacuac

SEQ ID NO: 1201 - RNA, 85 nt, Gallus gallus
cuagcaguaucaaagugcucauagugcagguagcuuggcauuggaccuacuguaaugugggcacuuacaguacuguuagauaaag

SEQ ID NO: 1202 - RNA, 74 nt, Xenopus tropicalis
gcaguuccaaagugcucauagugcagguaguuguauugauguucuacuguaauaugggcacuuacaguacugcu

SEQ ID NO: 1203 - RNA, 69 nt, Bos taurus
aguaccaaagugcucacagugcagguaguuuuggcagcgcucuacuguagugugggcacuuccaguacu

SEQ ID NO: 1204 - RNA, 71 nt, Sus scrofa; identical to SEQ ID NO: 1190

SEQ ID NO: 1205 - RNA, 94 nt, Danio rerio
gaguuuguccuggcaguuccaaagugcucacagugcagguagugccaguggaucuacugcaaugucugcacuucaaguauugccggacgccuuc

SEQ ID NO: 1206 - RNA, 80 nt, Fugu rubripes
agcaguauuaaagugcuuauagugcagguaguuacugauggucuacugcagugugagcacuucuaguacuucuagaugca

SEQ ID NO: 1207 - RNA, 73 nt, Tetraodon nigroviridis
gcaguauuaaagugcuuauagugcagguaguuuucucagggucuacugcaguaugagcacuucuaguacuucu

SEQ ID NO: 1208 - RNA, 65 nt, Mus musculus
augucaaagugcuaacagugcagguagcuuuuugaguucuacugcagugccagcacuucuuacau

SEQ ID NO: 1209 - RNA, 81 nt, Ateles geoffroyi
ccuuggccauguaaaagugcuuacagugcagguagcuuuuugagaucuacugcaaugcaagcacuucuuacauuaccaugg

SEQ ID NO: 1210 - RNA, 81 nt, Saguinus labiatus; identical to SEQ ID NO: 1209

SEQ ID NO: 1211 - RNA, 81 nt, Macaca mulatta; identical to SEQ ID NO: 1209

SEQ ID NO: 1212 - RNA, 81 nt, Macaca nemestrina; identical to SEQ ID NO: 1209

SEQ ID NO: 1213 - RNA, 81 nt, Gorilla gorilla; identical to SEQ ID NO: 1209

SEQ ID NO: 1214 - RNA, 81 nt, Pan paniscus; identical to SEQ ID NO: 1209

SEQ ID NO: 1215 - RNA, 81 nt, Pan troglodytes; identical to SEQ ID NO: 1209

SEQ ID NO: 1216 - RNA, 81 nt, Pongo pygmaeus; identical to SEQ ID NO: 1209

SEQ ID NO: 1217 - RNA, 81 nt, Gallus gallus
ccuugaguugugcaaaagugcuuacagugcagguagagcucagcaccuacugcaguauaagcacuucuggcaugaccgugg

SEQ ID NO: 1218 - RNA, 63 nt, Xenopus tropicalis
ugcaaaagugcuuauagugcagguagaauuuaaaccuacugcaccauaagcacuuccugcauc

SEQ ID NO: 1219 - RNA, 81 nt, Bos taurus; identical to SEQ ID NO: 1209

SEQ ID NO: 1220 - RNA, 81 nt, Sus scrofa
ccuuggccguguaaaagugcuuacagugcagguagcuuuuugagaucuacugcaaugcaagcacuucuuacauuaccaugg

SEQ ID NO: 1221 - RNA, 82 nt, Mus musculus
ccugcugggacuaaagugcugacagugcagauagugguccucucugugcuaccgcacuguggguacuugcugcuccagcagg

SEQ ID NO: 1222 - RNA, 82 nt, Rattus norvegicus; identical to SEQ ID NO: 1221

SEQ ID NO: 1223 - RNA, 82 nt, Ateles geoffroyi; identical to SEQ ID NO: 951

SEQ ID NO: 1224 - RNA, 81 nt, Lagothrix lagotricha
ccugccggggcuaaagugcugacagugcagauaguguccucuccgugcuaccgcacuguggguacuugcugcuccagcagg

SEQ ID NO: 1225 - RNA, 82 nt, Saguinus labiatus; identical to SEQ ID NO: 951

SEQ ID NO: 1226 - RNA, 82 nt, Macaca mulatta
ccugcuggggcuaaagugcugacagugcagauagugguccucuccgugcuaccgcacuguggguacuugcugcuccagcagg

SEQ ID NO: 1227 - RNA, 82 nt, Macaca nemestrina; identical to SEQ ID NO: 1226

SEQ ID NO: 1228 - RNA, 82 nt, Gorilla gorilla; identical to SEQ ID NO: 951

SEQ ID NO: 1229 - RNA, 82 nt, Pan paniscus; identical to SEQ ID NO: 951

SEQ ID NO: 1230 - RNA, 82 nt, Pan troglodytes; identical to SEQ ID NO: 951

SEQ ID NO: 1231 - RNA, 82 nt, Pongo pygmaeus; identical to SEQ ID NO: 951

SEQ ID NO: 1232 - RNA, 68 nt, Mus musculus
cuccggugccuacugagcugauaucaguucucauuucacacacuggcucaguucagcaggaacaggag

SEQ ID NO: 1233 - RNA, 107 nt, Mus musculus
gccucucuccgggcuccgccucccgugccuacugagcugaaacaguugauuccagugcacuggcucaguucagcaggaacaggaguccagcccccuaggagcuggca

SEQ ID NO: 1234 - RNA, 68 nt, Rattus norvegicus; identical to SEQ ID NO: 1232

SEQ ID NO: 1235 - RNA, 108 nt, Rattus norvegicus
gccucucccugggcuccgccuccugugccuacugagcugaaacaguugauuccagugcacuggcucaguucagcaggaacaggaguccagcccccauaggagcuggca

SEQ ID NO: 1236 - RNA, 68 nt, Macaca mulatta; identical to SEQ ID NO: 952

SEQ ID NO: 1237 - RNA, 73 nt, Macaca mulatta
cucugccucccgugccuacugagcugaaacacaguugguuugugcacacuggcucaguucagcaggaacagga

SEQ ID NO: 1238 - RNA, 68 nt, Macaca nemestrina; identical to SEQ ID NO: 952

SEQ ID NO: 1239 - RNA, 73 nt, Macaca nemestrina; identical to SEQ ID NO: 1237

SEQ ID NO: 1240 - RNA, 73 nt, Gorilla gorilla; identical to SEQ ID NO: 953

SEQ ID NO: 1241 - RNA, 68 nt, Pan paniscus; identical to SEQ ID NO: 952

SEQ ID NO: 1242 - RNA, 73 nt, Pan paniscus; identical to SEQ ID NO: 953

SEQ ID NO: 1243 - RNA, 73 nt, Pan troglodytes; identical to SEQ ID NO: 953

SEQ ID NO: 1244 - RNA, 68 nt, Pongo pygmaeus; identical to SEQ ID NO: 952

SEQ ID NO: 1245 - RNA, 73 nt, Pongo pygmaeus; identical to SEQ ID NO: 953

SEQ ID NO: 1246 - RNA, 57 nt, Monodelphis domestica
ggugccuacugagcugauaacaguucugauuuuacacacuggcucaguucagcagga

SEQ ID NO: 1247 - RNA, 73 nt, Monodelphis domestica
gccuccugugccuacugagcugaaacacaguugcuuuggauaaacuggcucaguucagcaggaacaggagucu

SEQ ID NO: 1248 - RNA, 68 nt, Gallus gallus
cuccggugccuacugagcugauaucaguucugauuuuacauacuggcucaguucagcaggaacaggag

SEQ ID NO: 1249 - RNA, 68 nt, Xenopus tropicalis
cucccguaccuacugggcugauaaucagugguuauaucuuccccuggcucaguucagcaggacaggag

SEQ ID NO: 1250 - RNA, 72 nt, Bos taurus
cucugccucccgugccuacugagcugaaacacaguugauuugugcacacuggcucaguucagcaggaacagg

SEQ ID NO: 1251 - RNA, 72 nt, Sus scrofa
cucugccucccgugccuacugagcugaaacacaguugauuugugcagacuggcucaguucagcaggaacagg

SEQ ID NO: 1252 - RNA, 80 nt, Danio rerio
accugagcuccggugccuucugagcugauaucaguuguaguaaaucacuggcucaguucagcaggaacaggaguguggcc

SEQ ID NO: 1253 - RNA, 82 nt, Danio rerio
gggcuggucuccugugccugcugugcugauaaucaguggacggcugugacuggcucaguucagcaggaacaggggccugguc

SEQ ID NO: 1254 - RNA, 142 nt, Danio rerio
gcugaacgggagucauaugagagauagauggagcuggccucccgugccuacugagcugauuacaguacugugcaaacacuggcucaguucagcaggaacaggagucugguccacaaacuccaccaaacacugcugcuucagc

SEQ ID NO: 1255 - RNA, 90 nt, Danio rerio
gagggcuguucccacuugugccugcuaaacugguaucaguauguugauuuagugcuggcucaguucagcaggaacaggugugaaguccuc

SEQ ID NO: 1256 - RNA, 79 nt, Fugu rubripes
ggguugugcccuccugugccuacugaacugguaucagugucauacuggaaaacuggcucaguucagcaggaacaggagu

SEQ ID NO: 1257 - RNA, 82 nt, Fugu rubripes
gggucagucuccugugccugcugugcugauaaucagugugugacguuggcuggcucaguucagcaggaacaggggacugguc

SEQ ID NO: 1258 - RNA, 70 nt, Tetraodon nigroviridis
cuuccugugccuacugagcugauaugcaguuguacagaaccacuggcucaguucagcaggaacaggaguc

SEQ ID NO: 1259 - RNA, 80 nt, Tetraodon nigroviridis
gggucagucuccugugccugcugugcugauaaucagugugugacguuggcuggcucaguucagcaggaacaggggacugg

SEQ ID NO: 1260 - RNA, 84 nt, Mus musculus; identical to SEQ ID NO: 954

SEQ ID NO: 1261 - RNA, 84 nt, Rattus norvegicus
ggccaguguugagaggcggagacacgggcaauugcuggacgcugcccugggcauugcacuugucucggucugacagugccggcc

SEQ ID NO: 1262 - RNA, 84 nt, Lagothrix lagotricha; identical to SEQ ID NO: 954

SEQ ID NO: 1263 - RNA, 84 nt, Macaca mulatta; identical to SEQ ID NO: 954

SEQ ID NO: 1264 - RNA, 84 nt, Macaca nemestrina; identical to SEQ ID NO: 954

SEQ ID NO: 1265 - RNA, 84 nt, Gorilla gorilla; identical to SEQ ID NO: 954

SEQ ID NO: 1266 - RNA, 84 nt, Pan paniscus; identical to SEQ ID NO: 954

SEQ ID NO: 1267 - RNA, 84 nt, Pan troglodytes
ggccaguguugagaggcggagacuugggcaauugcuggacgcugcccugggcauugcacuugucucggcugagacgcgcccgcc

SEQ ID NO: 1268 - RNA, 84 nt, Pongo pygmaeus; identical to SEQ ID NO: 954

SEQ ID NO: 1269 - RNA, 83 nt, Monodelphis domestica
ggccaguguugagaggcggagacuugggcaauugcugaacucugcccugggcauugcacuugucucggucugacagugcuggc

SEQ ID NO: 1270 - RNA, 82 nt, Xenopus tropicalis
gcugguguugacaggcagagacaggagcaacugcuggugugccuugguagcauugcacuugucucggucugacagugucggc

SEQ ID NO: 1271 - RNA, 82 nt, Xenopus tropicalis; identical to SEQ ID NO: 1270

SEQ ID NO: 1272 - RNA, 84 nt, Bos taurus
ggccaguguugagaggcggagacuugggcaauugcuggacgcugccccgggcauugcacuugucucggucugacagugccggcc

SEQ ID NO: 1273 - RNA, 82 nt, Danio rerio
gccggcgcugagaggcggagacuugggcagcugccgucauucccagaaggcauugcacuugucucggucugacaguggcggc

SEQ ID NO: 1274 - RNA, 82 nt, Fugu rubripes
gcugguguugagaggcggagacuugggcaauugccggccaucacagagggcauugcacuugucucggucugacagugccggc

SEQ ID NO: 1275 - RNA, 82 nt, Tetraodon nigroviridis; identical to SEQ ID NO: 1274

SEQ ID NO: 1276 - RNA, 70 nt, Mus musculus; identical to SEQ ID NO: 955

SEQ ID NO: 1277 - RNA, 70 nt, Rattus norvegicus
ggggauauugcacauuacuaaguugcauguugucacggccucaaugcaauuuagugugugugauauucuc

SEQ ID NO: 1278 - RNA, 70 nt, Saguinus labiatus; identical to SEQ ID NO: 955

SEQ ID NO: 1279 - RNA, 70 nt, Macaca mulatta; identical to SEQ ID NO: 955

SEQ ID NO: 1280 - RNA, 70 nt, Macaca nemestrina; identical to SEQ ID NO: 955

SEQ ID NO: 1281 - RNA, 70 nt, Gorilla gorilla; identical to SEQ ID NO: 955

SEQ ID NO: 1282 - RNA, 70 nt, Pan paniscus; identical to SEQ ID NO: 955

SEQ ID NO: 1283 - RNA, 70 nt, Pan troglodytes; identical to SEQ ID NO: 955

SEQ ID NO: 1284 - RNA, 70 nt, Pongo pygmaeus; identical to SEQ ID NO: 955

SEQ ID NO: 1285 - RNA, 69 nt, Monodelphis domestica
ggagauauugcacauuacuaaguugcauguugucacggcuuuagugcaauuuaguaugugugauauuuu

SEQ ID NO: 1286 - RNA, 70 nt, Gallus gallus
ggagauauugcacauuacuaaguugcauguugucacggccucagugcaauuuagugugugcgauacuuuc

SEQ ID NO: 1287 - RNA, 70 nt, Sus scrofa
ggagauauugcacauuacuaaguugcauguugucacggccucagugcaauuuagugugugugauauuuuc

SEQ ID NO: 1288 - RNA, 90 nt, Mus musculus
aaggccguggccucguucaaguaauccaggauaggcugugcaggucccaaggggccuauucuugguuacuugcacggggacgcgggccug

SEQ ID NO: 1289 - RNA, 84 nt, Mus musculus
ggcugcggcuggauucaaguaauccaggauaggcuguguccguccaugaggccuguucuugauuacuuguuucuggaggcagcg

SEQ ID NO: 1290 - RNA, 90 nt, Rattus norvegicus
aaggccguggccuuguucaaguaauccaggauaggcugugcaggucccaaggggccuauucuugguuacuugcacggggacgcgggccug

SEQ ID NO: 1291 - RNA, 77 nt, Lagothrix lagotricha; identical to SEQ ID NO: 956

SEQ ID NO: 1292 - RNA, 77 nt, Macaca mulatta; identical to SEQ ID NO: 956

SEQ ID NO: 1293 - RNA, 77 nt, Macaca nemestrina
guggccucguucaaguaauccaggauaggcugugcaggucccaaugggccuauucuugauuacuugcacggggacgc

SEQ ID NO: 1294 - RNA, 77 nt, Gorilla gorilla; identical to SEQ ID NO: 956

SEQ ID NO: 1295 - RNA, 77 nt, Pan paniscus; identical to SEQ ID NO: 956

SEQ ID NO: 1296 - RNA, 77 nt, Pan troglodytes; identical to SEQ ID NO: 956

SEQ ID NO: 1297 - RNA, 77 nt, Pongo pygmaeus; identical to SEQ ID NO: 956

SEQ ID NO: 1298 - RNA, 77 nt, Gallus gallus
gucaccugguucaaguaauccaggauaggcuguauccauuccugcuggccuauucuugguuacuugcacugggaggc

SEQ ID NO: 1299 - RNA, 84 nt, Bos taurus; identical to SEQ ID NO: 957

SEQ ID NO: 1300 - RNA, 84 nt, Sus scrofa; identical to SEQ ID NO: 957

SEQ ID NO: 1301 - RNA, 78 nt, Danio rerio
uuuggccugguucaaguaauccaggauaggcuugugauguccggaaagccuauucgggaugacuugguucaggaauga

SEQ ID NO: 1302 - RNA, 128 nt, Danio rerio
guguggacuugagugcuggaagugguuguucccuuguucaaguaauccaggauaggcugucuguccuggaggccuauucaugauuacuugcacuagguggcagccguugcccuucauggaacucaugc

SEQ ID NO: 1303 - RNA, 137 nt, Danio rerio
cuaagcugauacugagucaguguguggcugcaaccugguucaaguaauccaggauaggcuuuguggacuaggguuggccuguucuugguuacuugcacuggguugcagcuacuaaacaacuaagaagaucagaagag

SEQ ID NO: 1304 - RNA, 88 nt, Fugu rubripes
aggccucggccugguucaaguaauccaggauaggcugguuaacccugcacggccuauucuugauuacuugugucaggaaguggccgug

SEQ ID NO: 1305 - RNA, 96 nt, Tetraodon nigroviridis
gcguuaggccucggccugguucaaguaauccaggauaggcugguuaacccugcacggccuauucuugauuacuugugucaggaaguggccgccagc

SEQ ID NO: 1306 - RNA, 85 nt, Mus musculus
ugcccgggacccaguucaaguaauucaggauagguuguggugcugaccagccuguucuccauuacuuggcucgggggccggugcc

SEQ ID NO: 1307 - RNA, 85 nt, Rattus norvegicus
ugcccgggacccaguucaaguaauucaggauagguuguggugcuggccagccuguucuccauuacuuggcucgggggccggugcc

SEQ ID NO: 1308 - RNA, 85 nt, Bos taurus
ugcccgggacccaguucaaguaauucaggauagguugugugcuguccagccuguucuccauuacuuggcucgggggccggugccc

SEQ ID NO: 1309 - RNA, 97 nt, Danio rerio
gcauuuggccuuugccugguucaaguaauccaggauagguuaguucccacuaguacggccuauucuugguuacuuguuucaggaggaggcuacgagc

SEQ ID NO: 1310 - RNA, 73 nt, Mus musculus
aggugcagagcuuagcugauuggugaacagugauugguuuccgcuuuguucacaguggcuaaguucugcaccu

SEQ ID NO: 1311 - RNA, 87 nt, Rattus norvegicus
uggccuguggagcagggcuuagcugcuugugagcaaggucuacagcaaagucguguucacaguggcuaaguuccgcccccuggaccc

SEQ ID NO: 1312 - RNA, 97 nt, Rattus norvegicus; identical to SEQ ID NO: 959

SEQ ID NO: 1313 - RNA, 78 nt, Ateles geoffroyi
cugaggagcagggcuuagcugcuugugagcaggguucacuccaagucauguucacaguggcuaaguuccgccccccag

SEQ ID NO: 1314 - RNA, 78 nt, Saguinus labiatus; identical to SEQ ID NO: 1313

SEQ ID NO: 1315 - RNA, 78 nt, Macaca mulatta
cugaggagcagggcuuagcugcuugugagcaggguccacaccaagucguguucacaguggcuaaguuccgccccccag

SEQ ID NO: 1316 - RNA, 79 nt, Macaca nemestrina
cugaggagcagggcuuagcugcuugugagcaggguccacaccaagucguguucacaguggcuaaguuccgcccccccag

SEQ ID NO: 1317 - RNA, 78 nt, Gorilla gorilla; identical to SEQ ID NO: 1315

SEQ ID NO: 1318 - RNA, 78 nt, Pan paniscus; identical to SEQ ID NO: 1315

SEQ ID NO: 1319 - RNA, 78 nt, Pan troglodytes; identical to SEQ ID NO: 1315

SEQ ID NO: 1320 - RNA, 78 nt, Pongo pygmaeus
cugaggagcagggcuuagcugcuugugagcaggguccacaccaaguuguguucacaguggcuaaguuccgccccccag

SEQ ID NO: 1321 - RNA, 78 nt, Lemur catta
cugaggagcagggcuuagcugcuugugagcaggguccacaccaaaucauguucacaguggcuaaguuccgccccccgg

SEQ ID NO: 1322 - RNA, 86 nt, Monodelphis domestica
uggcuugaggagcaggacuuagcugccuugugaacagagucagcaucauauuguguucacaguggcuaaguuccgcuccccuugcc

SEQ ID NO: 1323 - RNA, 97 nt, Monodelphis domestica
accucucugacaaggugcagagcuuagccgauuggugaacagucacugauuuccucuuuguucacaguggcuaaguucugcaccugaagagaagggg

SEQ ID NO: 1324 - RNA, 97 nt, Gallus gallus
accucucuggugaggugcagagcuuagcugauuggugaacagugauuguuucccucuuuguucacaguggcuaaguucugcaccugaagagaaggug

SEQ ID NO: 1325 - RNA, 76 nt, Xenopus tropicalis
cggcggcccagggcuuagcuguauugugagcacugucacucucgcaccuguucacaguggcuaaguuccgcgccuc

SEQ ID NO: 1326 - RNA, 96 nt, Xenopus tropicalis
accuuucuaccaaggugcagagcuuagcugauuggugaacagugauugauucccucuuuguucacaguggcuaaguucugcaccugaagagagggc

SEQ ID NO: 1327 - RNA, 82 nt, Xenopus tropicalis
cucuagagaggcaggacuuagcuggcucugugaacaggucuugugugucaauguucacaguggcuaaguuccaccccacugg

SEQ ID NO: 1328 - RNA, 82 nt, Xenopus tropicalis; identical to SEQ ID NO: 1327

SEQ ID NO: 1329 - RNA, 78 nt, Bos taurus
uggccuggggagcagggcuuagcugcuugugagcagguccacaucaaaucguguucacaguggcuaaguuccgccccc

SEQ ID NO: 1330 - RNA, 97 nt, Bos taurus
accucucugacgaggugcagagcuuagcugauuggugaacagugacugguuuccgcuuuguucacaguggcuaaguucugcaccugaagagaaggug

SEQ ID NO: 1331 - RNA, 82 nt, Sus scrofa
uggccuggggagcagggcuuagcugcuugugagcagguccacagcaagucguguucacaguggcuaaguuccgcccccugga

SEQ ID NO: 1332 - RNA, 131 nt, Danio rerio
ucuggauaugaugucugcugaaguuucgugaggugcaggacuuagcucacucugugaacagaucucggauauccuauguucacaguggcuaaguuccgcuccucugaggcccacacucgaaaucagccagg

SEQ ID NO: 1333 - RNA, 91 nt, Danio rerio
ucuuuucuagcaggugcagagcuuagcugauuggugaacagugauugaacucuuuguucacaguggcuaaguucugcaucugaggagagga

SEQ ID NO: 1334 - RNA, 88 nt, Danio rerio
gguuguguggugucaggacuuaacccacuugugaacaaugcaucgaacuucaauguucacagugguuaaguucugccgccccuagacc

SEQ ID NO: 1335 - RNA, 89 nt, Danio rerio
uucugagcgggugcagagcuuggcugauuggugaacgugcauggcuuguguuuuuguucacaguggcuaaguucuucacccgaaaagaa

SEQ ID NO: 1336 - RNA, 106 nt, Danio rerio
gucaucacucgcucacggcgcagagcuuagcuaaucggugagcauugaucccuuaagaaaacuguucacaguggcuaaguucagugucuggagugaacgggaugac

SEQ ID NO: 1337 - RNA, 74 nt, Fugu rubripes
aggcacagagcuuagcugauuggugaacagugauugauuuccucuuuguucacaguggcuaaguucugcaccug

SEQ ID NO: 1338 - RNA, 78 nt, Fugu rubripes
ggcuguguggcggcaggacuuaacccacaugugagcagugagugugugccauguucacagugguuaaguucugccgcc

SEQ ID NO: 1339 - RNA, 100 nt, Fugu rubripes
cacuugcuaaaggcaccgagcuuagcuaauuggugagcauugaucccugcuauguguuguucacaguggcuaaguucagugccugaggugaaauaguuga

SEQ ID NO: 1340 - RNA, 74 nt, Tetraodon nigroviridis; identical to SEQ ID NO: 1337

SEQ ID NO: 1341 - RNA, 76 nt, Tetraodon nigroviridis
ggcugugcggcggcaggacuuaacccacaugugaacagugagugugccguguucacagugguuaaguucugccgcc

SEQ ID NO: 1342 - RNA, 83 nt, Tetraodon nigroviridis
cucuugcugaaggcacagagcuuagcuaauuggugagcauugaucccugcuauguguuguucacaguggcuaaguucagugcc

SEQ ID NO: 1343 - RNA, 86 nt, Mus musculus
ggucccuaccuucaaggagcucacagucuauugaguugccuuucugauucucccacuagauugugagcugcuggagggcaggcacu

SEQ ID NO: 1344 - RNA, 86 nt, Rattus norvegicus
ggucccuacccgcaaggagcucacagucuauugaguuccuuuucugauucucccacuagauugugagcuccuggagggcaggcacu

SEQ ID NO: 1345 - RNA, 86 nt, Ateles geoffroyi
gguccuugcccucaaggagcucacagucuauugaguugccuuucugacuuucccacuagauugugagcuccuggagggcaggcacu

SEQ ID NO: 1346 - RNA, 86 nt, Lagothrix lagotricha; identical to SEQ ID NO: 1345

SEQ ID NO: 1347 - RNA, 86 nt, Saguinus labiatus
gguccuugcccucaaggagcucacagucuauugaguugccuuucuaacuuucccacuagauuguaagcuccuggagggcaggcacu

SEQ ID NO: 1348 - RNA, 86 nt, Macaca mulatta
gguccuugcccucaaggagcucacagucuauugaguuuccuuugugacuuucccacuagauugugagcuccuggagggcaggcacu

SEQ ID NO: 1349 - RNA, 86 nt, Macaca nemestrina
gguccuugcccucaaggagcucacagucuauugaguuuccuuucugacuuucccacuagauugugagcuccuggagggcaggcacu

SEQ ID NO: 1350 - RNA, 86 nt, Gorilla gorilla; identical to SEQ ID NO: 960

SEQ ID NO: 1351 - RNA, 86 nt, Pan paniscus; identical to SEQ ID NO: 960

SEQ ID NO: 1352 - RNA, 86 nt, Pan troglodytes; identical to SEQ ID NO: 960

SEQ ID NO: 1353 - RNA, 86 nt, Pongo pygmaeus
gguccuugcccucaaggagcucacagucuauugaguuaccuuucugauuuucccacuagauugugagcuccuggagggcaggcacu

SEQ ID NO: 1354 - RNA, 86 nt, Sus scrofa
gguccuugcccucaaggagcucacacucuauugaguugccuuucugucuuccccacuagauugugagcuccuggagggcaggcacu

SEQ ID NO: 1355 - RNA, 88 nt, Mus musculus
accccuuagaggaugacugauuucuuuugguguucagagucaauagaauuuucuagcaccaucugaaaucgguuauaaugauugggga

SEQ ID NO: 1356 - RNA, 88 nt, Rattus norvegicus; identical to SEQ ID NO: 1355

SEQ ID NO: 1357 - RNA, 81 nt, Rattus norvegicus
cuucaggaagcugguuucauauggugguuuagauuuaaauagugauugucuagcaccauuugaaaucaguguucuuggugg

SEQ ID NO: 1358 - RNA, 81 nt, Rattus norvegicus
cuucuggaagcugguuucacaugguggcuuagauuuuuccaucuuuguaucuagcaccauuugaaaucaguguuuuaggag

SEQ ID NO: 1359 - RNA, 81 nt, Ateles geoffroyi
cuucaggaagcugguuucauauggugguuuagauuuaaauagugauugucuagcaccauuugaaaucaguguucuuggggg

SEQ ID NO: 1360 - RNA, 81 nt, Lagothrix lagotricha; identical to SEQ ID NO: 1359

SEQ ID NO: 1361 - RNA, 81 nt, Saguinus labiatus; identical to SEQ ID NO: 1358

SEQ ID NO: 1362 - RNA, 81 nt, Macaca nemestrina; identical to SEQ ID NO: 1358

SEQ ID NO: 1363 - RNA, 81 nt, Gorilla gorilla; identical to SEQ ID NO: 1359

SEQ ID NO: 1364 - RNA, 81 nt, Gorilla gorilla; identical to SEQ ID NO: 1358

SEQ ID NO: 1365 - RNA, 81 nt, Pan paniscus; identical to SEQ ID NO: 1359

SEQ ID NO: 1366 - RNA, 81 nt, Pan paniscus; identical to SEQ ID NO: 1358

SEQ ID NO: 1367 - RNA, 81 nt, Pan troglodytes; identical to SEQ ID NO: 1359

SEQ ID NO: 1368 - RNA, 81 nt, Pan troglodytes; identical to SEQ ID NO: 1358

SEQ ID NO: 1369 - RNA, 81 nt, Pongo pygmaeus; identical to SEQ ID NO: 1359

SEQ ID NO: 1370 - RNA, 81 nt, Pongo pygmaeus; identical to SEQ ID NO: 1358

SEQ ID NO: 1371 - RNA, 64 nt, Monodelphis domestica
augacugauuucuuuugguguucagagucaauaucauuuucuagcaccauuugaaaucgguuau

SEQ ID NO: 1372 - RNA, 86 nt, Monodelphis domestica
gcuucuucaggaagcugguuucauauggugguuuagauuuaacuacugagugucuagcaccauuugaaaucaguguucuuggagga

SEQ ID NO: 1373 - RNA, 89 nt, Gallus gallus
accccuuuagaggaugacugauuucuuuugguguucagagucaauaauauuuucuagcaccauuugaaaucgguuauagugauugggga

SEQ ID NO: 1374 - RNA, 81 nt, Gallus gallus
ccucaggaagcugguuucauauggugguuuagauuuaacuauucauugucuagcaccauuugaaaucaguguucuuggagg

SEQ ID NO: 1375 - RNA, 80 nt, Gallus gallus
ccucuggaagcugguuucacaugguggcuuagauuuucccacuuuguaucuagcaccauuugaaaucaguguucuaggag

SEQ ID NO: 1376 - RNA, 87 nt, Xenopus tropicalis
acccuuuuaaaggaugacugauuucuucugguguucagagucuuuuguuuucuagcaccauuugaaaucgguuauaaugauggggua

SEQ ID NO: 1377 - RNA, 80 nt, Xenopus tropicalis
ucccggaagcugguuucuuguggugacuuagauuuauccaucgcugcaucuagcaccauuugaaaucaguguucuaggag

SEQ ID NO: 1378 - RNA, 75 nt, Xenopus tropicalis
ucaggaggcugguuucauguggugguuuagauuuaacugcacaaugucuagcaccauaugaaaucaguguccuua

SEQ ID NO: 1379 - RNA, 81 nt, Bos taurus; identical to SEQ ID NO: 1358

SEQ ID NO: 1380 - RNA, 81 nt, Sus scrofa
cuucaggaagcugguuucauauggugguuuagauuuaaaaagugauugucuagcaccauuugaaaucaguguucuuggggg

SEQ ID NO: 1381 - RNA, 126 nt, Danio rerio
ugaagacccucaucucucucucucucuccccaccaaacgaugacugauuuccuuuggugcuuagagucccaucugucaucuagcaccauuugaaaucgguuauaaugacuggggaucaauucuuca

SEQ ID NO: 1382 - RNA, 71 nt, Danio rerio
agguugaccgauuucuuuugguguucagagucuuuugggguuucuagcaccauuugaaaucgguuacaaug

SEQ ID NO: 1383 - RNA, 83 nt, Danio rerio
cugcuccuggaagcugaauucagauggugccauagaguauuuuauggcaucuagcaccauuugaaaucaguguuccugggccg

SEQ ID NO: 1384 - RNA, 90 nt, Danio rerio
ucuccuccuggaagcugguuucaagugguguuuuagagugcugaucaaaugaaucuagcaccauuugaaaucaguguucuuggggugaga

SEQ ID NO: 1385 - RNA, 88 nt, Danio rerio
ucuuccuccagaugcugguuucacauggugguuuagauguguucuaccaaagucuagcaccauuugaaaucaguguucuuggggaggg

SEQ ID NO: 1386 - RNA, 71 nt, Fugu rubripes
agguugacugauuucuuuugguguucagaguccaauucuguuucuagcaccauuugaaaucgguuacagug

SEQ ID NO: 1387 - RNA, 126 nt, Fugu rubripes
gaagacagucuuggacucccuuccccguccuccaccaaaagaugacugauuucuucuggugcugagagucggcugcagccuucuagcaccauuugaaaucgguuauaaaacagugggucgauacau

SEQ ID NO: 1388 - RNA, 77 nt, Fugu rubripes
uccagaagcugguuucauguggugguuuagaugugucuucccucuugucuagcaccauuugaaaucaguguucuugg

SEQ ID NO: 1389 - RNA, 74 nt, Fugu rubripes
uggaagcugguuucaggugguggcuuagaguauugucaucuaucuagcaccauuugaaaucaguguucuugggg

SEQ ID NO: 1390 - RNA, 72 nt, Tetraodon nigroviridis
agguugaccgauuucuuuugguguucagaguccaauucuguuucuagcaccauuugaaaucgguuacaguga

SEQ ID NO: 1391 - RNA, 100 nt, Tetraodon nigroviridis
uguuucuccaccaaaagaugacugauuuccucuggugcuuagagccgccugcagccuucuagcaccauuugaaaucgguuauaaaacuguggaucagugc

SEQ ID NO: 1392 - RNA, 79 nt, Tetraodon nigroviridis
cuccagaagcugguuucacguggugguuuagaugugucuccucaucgucuagcaccauuugaaaucaguguucuugggg

SEQ ID NO: 1393 - RNA, 78 nt, Tetraodon nigroviridis
cucuuggaagcugguuucaggugguggcuuagaguauugucaucuaucuagcaccauuugaaaucaguguucuugggg

SEQ ID NO: 1394 - RNA, 88 nt, Mus musculus; identical to SEQ ID NO: 962

SEQ ID NO: 1395 - RNA, 88 nt, Rattus norvegicus; identical to SEQ ID NO: 962

SEQ ID NO: 1396 - RNA, 89 nt, Gallus gallus
ucucuuacacaggcugaccgauuucucuugguguucagagucucaguuucugucuagcaccauuugaaaucgguuaugauguaggggga

SEQ ID NO: 1397 - RNA, 90 nt, Xenopus tropicalis
ucccuuacacaggaugaccgaucucucuugguguucagaggcucaggucuucaucuagcaccauuugaaaucgguuauaauguaagguga

SEQ ID NO: 1398 - RNA, 88 nt, Bos taurus; identical to SEQ ID NO: 962

SEQ ID NO: 1399 - RNA, 88 nt, Sus scrofa
  misc_feature (83)..(83): n is a, c, g, or u
aucucuuacacaggcugaccgauuucuccugguguucagagucuguuuuugucuagcaccauuugaaaucgguuaugauguangggga

SEQ ID NO: 1400 - RNA, 96 nt, Mus musculus
cuaagccaaguuucaguucauguaaacauccuacacucagcugucauacaugcguuggcugggauguggauguuuacgucagcugucuuggaguau

SEQ ID NO: 1401 - RNA, 82 nt, Mus musculus
aagucugugucuguaaacauccccgacuggaagcuguaagccacagccaagcuuucagucagauguuugcugcuacuggcuc

SEQ ID NO: 1402 - RNA, 92 nt, Mus musculus
gggcagucuuugcuacuguaaacauccuugacuggaagcuguaagguguugagaggagcuuucagucggauguuuacagcggcaggcugcca

SEQ ID NO: 1403 - RNA, 71 nt, Rattus norvegicus
gcaacuguaaacauccucgacuggaagcugugaagccacaaaugggcuuucagucggauguuugcagcugc

SEQ ID NO: 1404 - RNA, 82 nt, Rattus norvegicus; identical to SEQ ID NO: 1401

SEQ ID NO: 1405 - RNA, 92 nt, Rattus norvegicus; identical to SEQ ID NO: 1402

SEQ ID NO: 1406 - RNA, 88 nt, Ateles geoffroyi
accgaguuucaguucauguaaacauccuacacucagcuguaauacauggauuggcugagagguggauguuuacuucagcugacuugga

SEQ ID NO: 1407 - RNA, 88 nt, Lagothrix lagotricha; identical to SEQ ID NO: 1406

SEQ ID NO: 1408 - RNA, 71 nt, Macaca mulatta
gcgacuguaaacauccucgacuggaagcugugaagccauagaugggcuuucagucggauguuugcagcugc

SEQ ID NO: 1409 - RNA, 88 nt, Macaca mulatta; identical to SEQ ID NO: 963

SEQ ID NO: 1410 - RNA, 88 nt, Macaca nemestrina; identical to SEQ ID NO: 963

SEQ ID NO: 1411 - RNA, 70 nt, Macaca nemestrina
guuguuguaaacauccccgacuggaagcuguaagacacagcuaagcuuucagucagauguuugcugcuac

SEQ ID NO: 1412 - RNA, 71 nt, Gorilla gorilla
gcgacuguaaacauccucgacuggaagcugugaagccacagaugggcuuucagucggauguuugcagcugc

SEQ ID NO: 1413 - RNA, 88 nt, Gorilla gorilla
accaaguuccaguucauguaaacauccuacacucagcuguaauacauggauuggcugggagguggauguuuacuucagcugacuugga

SEQ ID NO: 1414 - RNA, 70 nt, Gorilla gorilla; identical to SEQ ID NO: 1411

SEQ ID NO: 1415 - RNA, 71 nt, Pan paniscus; identical to SEQ ID NO: 1412

SEQ ID NO: 1416 - RNA, 88 nt, Pan paniscus; identical to SEQ ID NO: 963

SEQ ID NO: 1417 - RNA, 70 nt, Pan paniscus; identical to SEQ ID NO: 1411

SEQ ID NO: 1418 - RNA, 71 nt, Pan troglodytes; identical to SEQ ID NO: 1412

SEQ ID NO: 1419 - RNA, 88 nt, Pan troglodytes; identical to SEQ ID NO: 963

SEQ ID NO: 1420 - RNA, 70 nt, Pan troglodytes; identical to SEQ ID NO: 1411

SEQ ID NO: 1421 - RNA, 71 nt, Pongo pygmaeus; identical to SEQ ID NO: 1412

SEQ ID NO: 1422 - RNA, 72 nt, Monodelphis domestica
gcggcuguaaacauccucgacuggaagcugugaagcagcagauggggcuuucagucggauguuugcagcugc

SEQ ID NO: 1423 - RNA, 72 nt, Gallus gallus
gcgacuguaaacauccucgacuggaagcugugaagcagcagauggggcuuucagucggauguuugcagcugc

SEQ ID NO: 1424 - RNA, 87 nt, Gallus gallus
cuaacuuuuaguuccuguaaacauccuacacucagcuauaacaagugguagggcugggggguggauguuuacuucaacugacuugga

SEQ ID NO: 1425 - RNA, 64 nt, Gallus gallus
guugcuguaaacauccccgacuggaagcuguagcagcuugagcuuucagucagauguuugcugc

SEQ ID NO: 1426 - RNA, 96 nt, Gallus gallus
cggggcagucaucgcugcuguaaacauccuugacuggaagcugugaggugucagcgggggcuuucagucggauguuuacagcugcaggcugcugca

SEQ ID NO: 1427 - RNA, 71 nt, Xenopus tropicalis
gcgacuguaaacauccucgacuggaagcugugaagcaguugaagggcuuucagucagauguuugcagcugc

SEQ ID NO: 1428 - RNA, 80 nt, Xenopus tropicalis
guauuagucuauguaaacauccuacacucagcucguauguauaaccugacugggugggggguguuugccucgacugaucu

SEQ ID NO: 1429 - RNA, 81 nt, Xenopus tropicalis
aguuugucgcuguaaacauccccgacuggaagcugugaggcugcauuugagcuuucagucugguguuugcugcuaccggcu

SEQ ID NO: 1430 - RNA, 90 nt, Xenopus tropicalis
gggcaguuacagccucuguaaacauccuugacuggaagcugugaaauaaugauaguagcuuucagucggauguuuacagcugcuagcugc

SEQ ID NO: 1431 - RNA, 67 nt, Bos taurus
cuguaaacauccucgacuggaagcugugaggcugcagaaaggcuuucagucggauguuugcagcugc

SEQ ID NO: 1432 - RNA, 88 nt, Bos taurus
ccaaguuuucaguucauguaaacauccuacacucagcuguaacacacgagucggcugggagguggauguuuacuucagcugacuugga

SEQ ID NO: 1433 - RNA, 70 nt, Bos taurus
guuguuguaaacauccccgacuggaagcuguaccacacagcuaagcuuucagucagauguuugcugcuac

SEQ ID NO: 1434 - RNA, 92 nt, Bos taurus
gggcagucuuugcuacuguaaacauccuugacuggaagcuguaaggcguugcaaggagcuuucagucggauguuuacagcggcaggcugcca

SEQ ID NO: 1435 - RNA, 89 nt, Danio rerio
ggcuccuugcaguuguaaacauucccgacuggaaguuguaaugcagaaaaucucagcuuucagucugauguuugcugcuacugguggcc

SEQ ID NO: 1436 - RNA, 88 nt, Danio rerio
uuccaguguagucgcuguaaacauccuacacucagcugugagcugcagacgaggcugggcggaggguguuugcugugacugucuggag

SEQ ID NO: 1437 - RNA, 128 nt, Danio rerio
gugcuguauauggguauaacugguuguucaugccuguaaacauccccgacuggaagcugugcuacgcggaaaacgagcuuucaguuggauguuugcugucaucguccaguucugucgccuuguauuac

SEQ ID NO: 1438 - RNA, 80 nt, Danio rerio
uacgggcuacuguaaacauccuugacuggaagcuggugcacaugauggagcuuucagucggauguuugcagcagccaacu

SEQ ID NO: 1439 - RNA, 90 nt, Fugu rubripes
uuccggcucaguugguguaaacauccuacacucagcugugauccuuugcggcagaggcugagagaagguuguuuacuugaacuggcugga

SEQ ID NO: 1440 - RNA, 98 nt, Fugu rubripes
ugcacauggcaguuugaggcuguaaacauccccgacuggaagcugucacucagaagagcuuucagucugauguuuaccaccuccgaccgccuccugcu

SEQ ID NO: 1441 - RNA, 81 nt, Tetraodon nigroviridis
agaugguguaaacauccuacacucagcugugaucuuuugccgcagaggcugagagaagguuguuuacuugaacuggcugga

SEQ ID NO: 1442 - RNA, 86 nt, Tetraodon nigroviridis
uggcaguuugaggcuguaaacauccccgacuggaagcugucacucagcagagcuuucagucugauguuuacccccuccgaccgccu

SEQ ID NO: 1443 - RNA, 89 nt, Mus musculus
accauguuguaguguguguaaacauccuacacucucagcugugagcucaagguggcugggagaggguuguuuacuccuucugccaugga

SEQ ID NO: 1444 - RNA, 84 nt, Mus musculus
gagugacagauauuguaaacauccuacacucucagcugugaaaaguaagaaagcugggagaaggcuguuuacucucucugccuu

SEQ ID NO: 1445 - RNA, 89 nt, Rattus norvegicus; identical to SEQ ID NO: 1443

SEQ ID NO: 1446 - RNA, 84 nt, Rattus norvegicus
gagugacagauacuguaaacauccuacacucucagcugugaaaaguaagaaagcugggagaaggcuguuuacucucucugccuu

SEQ ID NO: 1447 - RNA, 72 nt, Lagothrix lagotricha; identical to SEQ ID NO: 965

SEQ ID NO: 1448 - RNA, 72 nt, Macaca nemestrina; identical to SEQ ID NO: 965

SEQ ID NO: 1449 - RNA, 72 nt, Pan troglodytes; identical to SEQ ID NO: 965

SEQ ID NO: 1450 - RNA, 89 nt, Gallus gallus
accaugcuguagcacguguaaacauccuacacucucagcugugaacucgagguggcugggagaggauuguuuacgccuucugccaugga

SEQ ID NO: 1451 - RNA, 72 nt, Gallus gallus
agguacuguaaacauccuacacucucagcuguggaaacuaagaaagcugggagaaggcuguuuacucucccu

SEQ ID NO: 1452 - RNA, 89 nt, Xenopus tropicalis
accaugcagcagugauuguaaacauccuacacucucagcugugaacauaagguggcugggagaaggguguuuacucccccugccaugga

SEQ ID NO: 1453 - RNA, 78 nt, Xenopus tropicalis
gagagacagauacuguaaacauccuacacucucagcuguggaaauacaaagcugggggaaggcuguuuacucucucug

SEQ ID NO: 1454 - RNA, 105 nt, Bos taurus
cagacuguaaccaugccguaguguguguaaacauccuacacucucagcugugagcucgagguggcugggagaggguuguuuacuccuucugccauggaaaacguc

SEQ ID NO: 1455 - RNA, 80 nt, Sus scrofa
gacagauacuguaaacauccuacacucucagcuguggaaaguaagaaagcugggagaaggcuguuuacucucucugccuu

SEQ ID NO: 1456 - RNA, 79 nt, Danio rerio
cuucagggaguguaaacauccuacacucucagcuggagcgcagccgaggccgggagugggauguuugcgcucucuggcu

SEQ ID NO: 1457 - RNA, 78 nt, Fugu rubripes
ucaggaaguguaaacauccuacacucucggcguccgcccccugguggccgggggugggacuguuugcacugccugacu

SEQ ID NO: 1458 - RNA, 79 nt, Tetraodon nigroviridis
uucagggaguguaaacauccuacacucucggcguccgcccccugguggccgggggugggacuguuugcacugccuggcu

SEQ ID NO: 1459 - RNA, 80 nt, Ateles geoffroyi
cugggggcuccaaagugcuguucgugcagguagugugauuaccugaccuacugcugagcuagcacuucccgagcccccgg

SEQ ID NO: 1460 - RNA, 80 nt, Lagothrix lagotricha
cucggggcuccaaagugcuguucgugcagguagugugauuaccugaccuacugcugagcuagcacuucccgagcccccgg

SEQ ID NO: 1461 - RNA, 80 nt, Saguinus labiatus; identical to SEQ ID NO: 966

SEQ ID NO: 1462 - RNA, 80 nt, Macaca mulatta
cugggggcuccaaagugcuguucgugcagguagugugauuaaccgaccuacugcugagcuagcacuucccgagcccccag

SEQ ID NO: 1463 - RNA, 80 nt, Macaca nemestrina; identical to SEQ ID NO: 1462

SEQ ID NO: 1464 - RNA, 80 nt, Gorilla gorilla
cugggggcuccaaagugcuguucgugcagguagugugauuacccgaccuacugcugagcuagcacuucccgagcccccgg

SEQ ID NO: 1465 - RNA, 80 nt, Pan paniscus; identical to SEQ ID NO: 1464

SEQ ID NO: 1466 - RNA, 80 nt, Pan troglodytes; identical to SEQ ID NO: 1464

SEQ ID NO: 1467 - RNA, 80 nt, Pongo pygmaeus; identical to SEQ ID NO: 1464

SEQ ID NO: 1468 - RNA, 89 nt, Monodelphis domestica
agucuuggggggcuccaaagugcuguucgugcagguagugugauaaccugaccuacugcugagcuagcacuuccagagccccugggaca

SEQ ID NO: 1469 - RNA, 74 nt, Mus musculus
guucccuucaacuuuaacaugggaaugcuuucugucucaucgaagaguaagugcuuccauguuuuaguagaagu

SEQ ID NO: 1470 - RNA, 66 nt, Mus musculus
ccuuuacuuuaacauggaggcacuugcugugcauuuaaaaauaagugcuuccauguuugagugugg

SEQ ID NO: 1471 - RNA, 70 nt, Monodelphis domestica
cccacuacuuaaacguggauuuacuugcuuuguuucuaaaaaaguaagugcuuccauguuuuagugaugg

SEQ ID NO: 1472 - RNA, 71 nt, Monodelphis domestica
ccccuucuacuuuaacauggagguacuuucuguguuuaaaaagguaagugcuuccauguuuugguaggagu

SEQ ID NO: 1473 - RNA, 59 nt, Monodelphis domestica
gcuuuaacauggggguaccugcuacguaauaaaaaguaagugcuuccauguuucagugg

SEQ ID NO: 1474 - RNA, 71 nt, Monodelphis domestica
ccucugcuuuaacauggaagugcuugcugugauuuuaaaaaauuaagugcuuccauguuugaguuguggug

SEQ ID NO: 1475 - RNA, 72 nt, Gallus gallus
cccuuuuacuuuaacauggaggugcuuucugugauuuuacaaaaguaagugcuuccauguuuuaguagaggu

SEQ ID NO: 1476 - RNA, 65 nt, Gallus gallus
ccuccgcuuuaacauggagguaccugcugccuacaaaaguaagugcuuccauguuucaguggugg

SEQ ID NO: 1477 - RNA, 69 nt, Gallus gallus
ccucaacuuuaacauggagguacuugcuggacaccugaaaaaguaagugcuuccauguuuuaguugugg

SEQ ID NO: 1478 - RNA, 69 nt, Xenopus tropicalis
ccccacuacuuuaacauugguguacuuucuaugucuuuaaaaaggguaagugcuccaauguuuuagugg

SEQ ID NO: 1479 - RNA, 81 nt, Lagothrix lagotricha; identical to SEQ ID NO: 968

SEQ ID NO: 1480 - RNA, 81 nt, Saguinus labiatus; identical to SEQ ID NO: 968

SEQ ID NO: 1481 - RNA, 81 nt, Gorilla gorilla; identical to SEQ ID NO: 968

SEQ ID NO: 1482 - RNA, 81 nt, Pan paniscus
aacacagugggcacucaauaaaugucuguugaauugaaaugcauuacauucaacggguauuuauugagcacccacucugug

SEQ ID NO: 1483 - RNA, 81 nt, Pan troglodytes; identical to SEQ ID NO: 968

SEQ ID NO: 1484 - RNA, 81 nt, Pongo pygmaeus; identical to SEQ ID NO: 968

SEQ ID NO: 1485 - RNA, 81 nt, Sus scrofa
aacacagugggcgcucaauaaauguuuguugaauugagaugcguuaaauucaacggguauuuauugagcacccacucugug

SEQ ID NO: 1486 - RNA, 108 nt, Mus musculus
cugcacaugcuggggugagguaguaaguuguauuguugugggguagggauuuuaggccccaguaagaagauaacuauacaacuuacuacuuuccuugguguguggcau

SEQ ID NO: 1487 - RNA, 108 nt, Rattus norvegicus
cugcacaugcuggggugagguaguaaguuguauuguugugggguagggauuuuaggccccaauaagaagauaacuauacaacuuacuacuuucccugguguguggcau

SEQ ID NO: 1488 - RNA, 80 nt, Ateles geoffroyi
gugagguaguaaguuguauuguugugggguagggauauuaggccccaauuagaagauaacuauacaacuuacuacuuucc

SEQ ID NO: 1489 - RNA, 80 nt, Macaca mulatta; identical to SEQ ID NO: 1488

SEQ ID NO: 1490 - RNA, 80 nt, Gorilla gorilla; identical to SEQ ID NO: 1488

SEQ ID NO: 1491 - RNA, 80 nt, Pan paniscus; identical to SEQ ID NO: 1488

SEQ ID NO: 1492 - RNA, 119 nt, Pan troglodytes; identical to SEQ ID NO: 969

SEQ ID NO: 1493 - RNA, 80 nt, Pongo pygmaeus; identical to SEQ ID NO: 1488

SEQ ID NO: 1494 - RNA, 108 nt, Xenopus tropicalis
ucugcccucuucaggaugagguaguaaguuguauuguuguggggguuuuuauuuccccaaauuaggagauaacuauacagcuuacugccuucccugcagcaugguaau

SEQ ID NO: 1495 - RNA, 119 nt, Bos taurus
aggacucugcucaugcuggggugagguaguaaguuguauuguugugggguagggauuuuaggccccaauuugaagauaacuauacaacuuacuacuuucccugguguguagcacauuca

SEQ ID NO: 1496 - RNA, 64 nt, Mus musculus
gagcucuuuucacauugugcuacugucuaacguguaccgagcagugcaauguuaaaagggcauc

SEQ ID NO: 1497 - RNA, 88 nt, Rattus norvegicus
ugcugcuggccggagcucuuuucacauugugcuacugucuacacguguaccgagcagugcaauguuaaaagggcaucggccuuguagu

SEQ ID NO: 1498 - RNA, 89 nt, Macaca mulatta
ugcugcuggccagagcucuuuucacauugugcuacugucugcaccugucacuagcagugcaauguuaaaagggcauuggccccccagua

SEQ ID NO: 1499 - RNA, 89 nt, Macaca nemestrina; identical to SEQ ID NO: 970

SEQ ID NO: 1500 - RNA, 89 nt, Gorilla gorilla; identical to SEQ ID NO: 970

SEQ ID NO: 1501 - RNA, 89 nt, Pan paniscus; identical to SEQ ID NO: 970

SEQ ID NO: 1502 - RNA, 76 nt, Monodelphis domestica
uggccagagcucuuuucacauugugcuacugucugcaccuaucacuagcagugcaauguaaaaagggcauuggcug

SEQ ID NO: 1503 - RNA, 83 nt, Gallus gallus
ugcuguuguccagagcccuuuuucuguuguacuacuggcaauuaugaugagcagugcaauauuaaaagggcauuggcuggcag

SEQ ID NO: 1504 - RNA, 83 nt, Xenopus tropicalis
cuguccagugcccuuuuuauguuguacuacuaguggucacacacaaaaaaagcagugcaauguuaaaagggcauuggccaggg

SEQ ID NO: 1505 - RNA, 85 nt, Danio rerio
ugucuguccagugccccuuuuauauuguacuacugauaacccaguuauuaaagcagugcaauguuaaaagggcauuggccaggga

SEQ ID NO: 1506 - RNA, 85 nt, Danio rerio; identical to SEQ ID NO: 1505

SEQ ID NO: 1507 - RNA, 141 nt, Danio rerio
gggagaggucuguacggugauauugaccauguuuuguccauugcccuuuuucuguuguacuacuggccaaucagaagagcagugcaauauuaaaagggcauuggcugauagaacagaguccuucaccucucacccucuccu

SEQ ID NO: 1508 - RNA, 81 nt, Danio rerio
uuguugaccagggcccuuuuucuguuguacuacugugcagucagaugagcagugcaauauuaaaagggcauuggcugacaa

SEQ ID NO: 1509 - RNA, 79 nt, Fugu rubripes
uguuguuguccucugcccuuuuucuguugcaccacuggacacugagaugagcagugcaauauuaaaagggcauuggcug

SEQ ID NO: 1510 - RNA, 74 nt, Tetraodon nigroviridis
uguuguccucugcccuuuuucuguugcaccacuggacacugagaugagcagugcaauauuaaaagggcauuggc

SEQ ID NO: 1511 - RNA, 82 nt, Mus musculus
ggcuuguuggacacucuuucccuguugcacuacugugggccucugggaagcagugcaaugaugaaagggcaucugucgggcc

SEQ ID NO: 1512 - RNA, 82 nt, Rattus norvegicus
ggcuugcuggacacucuuucccuguugcacuacugugggccucugggaagcagugcaaugaugaaagggcauccgucaggcc

SEQ ID NO: 1513 - RNA, 77 nt, Gallus gallus
gggcgggcugccccucuuucccuguugcacuacugucacggucgcagcgagcagugcaauaaugaaagggcgucagu

SEQ ID NO: 1514 - RNA, 76 nt, Xenopus tropicalis
ccgccugacacucuuucccuguugcacuacuguggcagugaauaaagcagugcaaugaugaaagggcaucagucug

SEQ ID NO: 1515 - RNA, 84 nt, Danio rerio
cuguugccugacacucuuucccuguugcacuacugugggagcugcagcaaagcagugcaauaaugaaagggcaucaguccacug

SEQ ID NO: 1516 - RNA, 86 nt, Mus musculus
ccugcuaacggcugcucugacuuuauugcacuacuguacuuuacagcgagcagugcaauaguauugucaaagcauccgcgagcagg

SEQ ID NO: 1517 - RNA, 97 nt, Mus musculus
uuuccugcuggcugcgggugcucugacuagguugcacuacugugcugugagaagcagugcaaugguauugucaaagcaucugggaccagccucgaag

SEQ ID NO: 1518 - RNA, 100 nt, Rattus norvegicus
ccugcuggcuacugcugacgacugcucugacuuuauugcacuacuguacuguacagcuagcagugcaauaguauugucaaagcauccgggagcaggcuac

SEQ ID NO: 1519 - RNA, 78 nt, Rattus norvegicus
gcugcgggugcucugacuagguugcacuacugugcuguggaaagcagugcaaugguauugucaaagcaucugggacca

SEQ ID NO: 1520 - RNA, 93 nt, Gallus gallus
uggcugcugguaucgcuggcucugacaauguugcacuacugucugcacaaauaaagcagugcaauaauauugucaaagcauuugguuccaguc

SEQ ID NO: 1521 - RNA, 84 nt, Xenopus tropicalis
cugcuaaugugugcucugacuuuauugcacuacuguacuauacagcuagcagugcaauaguauugucaaagcaucugcaagcag

SEQ ID NO: 1522 - RNA, 73 nt, Xenopus tropicalis
gaauugcucugacaguguugcacuacugugaucucaaaugaagcagugcaauaguauugucaaagcauuucau

SEQ ID NO: 1523 - RNA, 86 nt, Danio rerio
gcuguuaacaggugcucugacuucauugcacuacuguauuggacagcuagcagugcaauaguauugucaaagcgucugagagcagc

SEQ ID NO: 1524 - RNA, 86 nt, Danio rerio; identical to SEQ ID NO: 1523

SEQ ID NO: 1525 - RNA, 81 nt, Danio rerio
aaggucuguugcuuugacgauguugcacuacugaaccaucuaaucaagcagugcaauaguauugucauugcauucggcuuu

SEQ ID NO: 1526 - RNA, 83 nt, Danio rerio
ugaggucagcugcucugacgauguugcacuacuguaccauccauucuagcagugcaauaguauugucauagcauuuagccucu

SEQ ID NO: 1527 - RNA, 68 nt, Fugu rubripes
cagcugcuuugacaauguugcacuacuguaccauccauucuagcagugcaauaguauugucauagcau

SEQ ID NO: 1528 - RNA, 79 nt, Tetraodon nigroviridis
aggucagcugcucugacaauguugcacuacuguaccauccauucuagcagugcaauaguauugucauagcauuuggccu

SEQ ID NO: 1529 - RNA, 66 nt, Mus musculus
gggcaaccguggcuuucgauuguuacugugggaaccggagguaacagucuacagccauggucgccc

SEQ ID NO: 1530 - RNA, 101 nt, Rattus norvegicus
ccgcccccgcgucuccagggcaaccguggcuuucgauuguuacugugggaaccggagguaacagucuacagccauggucgccccgcagcacgcccacgcuc

SEQ ID NO: 1531 - RNA, 66 nt, Monodelphis domestica
gggcaaccguggcuuucgauuguuacugugggaaccagggguaacagucuacagccauggucgccc

SEQ ID NO: 1532 - RNA, 87 nt, Xenopus tropicalis
cugucuccagggcaaccguggcuuuagauuguuacuguaguucugcauugguaacagucuacagccauggucgcucgggcaagaugc

SEQ ID NO: 1533 - RNA, 101 nt, Bos taurus
ccgcccccgcgucuccagggcaaccguggcuuucgauuguuacugugggaaccggagguaacagucuacagccauggucgccccgcagcacgcccacgcgc

SEQ ID NO: 1534 - RNA, 85 nt, Danio rerio
gucuccauggcgaccguggcauuagauuguuacuguaggaacagaauuuuugguaacagucuacagccauggucgcuagugggca

SEQ ID NO: 1535 - RNA, 119 nt, Danio rerio
ucccuuccucugcugucuccaugcugaccguggcuuuagauuguuacuguagcguuggcacgugguaacagucuacagccauggucgccaaggggcagguguuacagcgacuacuggga

SEQ ID NO: 1536 - RNA, 85 nt, Fugu rubripes
gucuccaugguuaccguggcauuagauuguuacuguagcaacugcaccagugguaacagucuacagccauggucgcuagggggca

SEQ ID NO: 1537 - RNA, 85 nt, Tetraodon nigroviridis
gucuccaugguuaccguggcauuagauuguuacuguagcaacugcaccagcgguaacagucuacagccauggucgcuagggggca

SEQ ID NO: 1538 - RNA, 91 nt, Mus musculus
gggcagcgcgccggcaccuuggcucuagacugcuuacugcccgggccgccuucaguaacagucuccagucacggccaccgacgccuggccc

SEQ ID NO: 1539 - RNA, 111 nt, Rattus norvegicus
cgggauauccccgcccgggcagcgcgccggcaccuuggcucuagacugcuuacugcccgggccgcccucaguaacagucuccagucacggccaccgacgccuggccccgcc

SEQ ID NO: 1540 - RNA, 91 nt, Monodelphis domestica
gggcagcgcgccggcaccuuggcucuagacugcuuacugcccgggccacccucaguaacagucuccagucacggccaccgacgccuggccc

SEQ ID NO: 1541 - RNA, 96 nt, Xenopus tropicalis
uggucauugcaucggcaccuuggcucuagacugcuuacugugaaacugugcuauaggaacaguaacagucuacagucauggcuacugacguaugac

SEQ ID NO: 1542 - RNA, 94 nt, Danio rerio
gucagugcaucaauaccuuggcucuagacugcuuacugcuaaaugugucugaaaguacaguaacagucuacagucauggcuacugacgucuggc

SEQ ID NO: 1543 - RNA, 95 nt, Fugu rubripes
ggucagcgcaucaacaccuuggcucuagacugcuuacugcuaaaaccgcucauaaguacaguaacagucuacagucauggcuacugacgccugac

SEQ ID NO: 1544 - RNA, 95 nt, Tetraodon nigroviridis
ggucagcgcaucaacaccuuggcucuagacugcuuacugcuaacaccgcucugaaguacaguaacagucuacagucauggcuacugacgccuggc

SEQ ID NO: 1545 - RNA, 88 nt, Ateles geoffroyi; identical to SEQ ID NO: 975

SEQ ID NO: 1546 - RNA, 88 nt, Lagothrix lagotricha; identical to SEQ ID NO: 975

SEQ ID NO: 1547 - RNA, 88 nt, Saguinus labiatus; identical to SEQ ID NO: 975

SEQ ID NO: 1548 - RNA, 88 nt, Macaca nemestrina; identical to SEQ ID NO: 975

SEQ ID NO: 1549 - RNA, 88 nt, Gorilla gorilla; identical to SEQ ID NO: 975

SEQ ID NO: 1550 - RNA, 88 nt, Pan paniscus; identical to SEQ ID NO: 975

SEQ ID NO: 1551 - RNA, 88 nt, Pan troglodytes; identical to SEQ ID NO: 975

SEQ ID NO: 1552 - RNA, 87 nt, Monodelphis domestica
caaugcuuugcuaaagcugguaaaauggaaccaaaucaccuauucaauggauuugguccccuucaaccagcuguagcuaugcauuga

SEQ ID NO: 1553 - RNA, 87 nt, Gallus gallus
caaugcuuugcuaaagcugguaaaauggaaccaaaucaccucuucaauggauuugguccccuucaaccagcuguagcuaugcauuga

SEQ ID NO: 1554 - RNA, 96 nt, Gallus gallus
gugaggccaaaugcuuugcuaaagcugguaaaauggaaccaaaucaacuguucaauggauuugguccccuucaaccagcuguagcugugcauugau

SEQ ID NO: 1555 - RNA, 79 nt, Gallus gallus
ugccuuccuggggcugguaaaaaggaaccagaucaacuacaacuggauuugguccccuucaaccagcugcaguggggca

SEQ ID NO: 1556 - RNA, 86 nt, Xenopus laevis
cagugcuuugcuaaagcugguaaaauggaaccaaaucaccucuucaauggauuugguccccuucaaccagcuguaggauugcauug

SEQ ID NO: 1557 - RNA, 86 nt, Xenopus tropicalis
caaugcuuugcuaaagcugguaaaauggaaccaaaucaccucuucaauggauuugguccccuucaaccagcuguaggauugcauug

SEQ ID NO: 1558 - RNA, 76 nt, Xenopus tropicalis
cugguugcggcuggugaaaaggaaccacaucaacccagaaaaaggauuugguccccuucaaccagccgcaacuggu

SEQ ID NO: 1559 - RNA, 84 nt, Gallus gallus
cucugcucuggcuggucaaacggaaccaagcccgucuucuucggagguuugguccccuucaaccagcuauagcaguguugaaaa

SEQ ID NO: 1560 - RNA, 75 nt, Xenopus tropicalis
cugcuauggcuggucaaacggaaccaaguccgucuuccuuagagguuugguccccuucaaccagcuauugcagua

SEQ ID NO: 1561 - RNA, 71 nt, Mus musculus
agggugugugacugguugaccagaggggcgugcacucuguucacccugugggccaccuagucaccaacccu

SEQ ID NO: 1562 - RNA, 73 nt, Rattus norvegicus
cagggugugugacugguugaccagaggggcgugcacuuuguucacccugugggccaccuagucaccaacccuc

SEQ ID NO: 1563 - RNA, 73 nt, Macaca nemestrina; identical to SEQ ID NO: 978

SEQ ID NO: 1564 - RNA, 73 nt, Gorilla gorilla
uagggugugugacugguugaccagaggggcaugcacuguguucacccugugggccaccuagucaccaacccuc

SEQ ID NO: 1565 - RNA, 73 nt, Pan paniscus; identical to SEQ ID NO: 978

SEQ ID NO: 1566 - RNA, 73 nt, Pongo pygmaeus; identical to SEQ ID NO: 978

SEQ ID NO: 1567 - RNA, 56 nt, Monodelphis domestica
cuuggguggauaauacggauuacguuguuauugcuuaagaauacgcguagucgagg

SEQ ID NO: 1568 - RNA, 96 nt, Gallus gallus
ucugacucucuucggugacggguauucuuggguggauaauacggauuacguuguuauugcuuaagaauacgcguagucgaggagaguaccggcggc

SEQ ID NO: 1569 - RNA, 101 nt, Xenopus tropicalis
gcucuguggcucucuucggugacggguauucuuggguggauaauacggauuacguuguuauugcuuaagaauacgcguaguugaggagaguaucuucagca

SEQ ID NO: 1570 - RNA, 101 nt, Xenopus tropicalis; identical to SEQ ID NO: 1569

SEQ ID NO: 1571 - RNA, 101 nt, Xenopus tropicalis; identical to SEQ ID NO: 1569

SEQ ID NO: 1572 - RNA, 64 nt, Mus musculus
acccauaaaguagaaagcacuacuaacagcacuggaggguguaguguuuccuacuuuauggaug

SEQ ID NO: 1573 - RNA, 87 nt, Rattus norvegicus; identical to SEQ ID NO: 980

SEQ ID NO: 1574 - RNA, 88 nt, Gallus gallus
gacagugcagucacccauaaaguagaaagcacuacuaaacagcacugcaggguguaguguuuccuacuuuauggaugaguguacugug

SEQ ID NO: 1575 - RNA, 86 nt, Xenopus tropicalis
cagugcagccacccauaaaguagaaagcacuacuagacaggacugaacgcuguaguguuuccuacuuuauggaugaguguacuggg

SEQ ID NO: 1576 - RNA, 86 nt, Xenopus tropicalis; identical to SEQ ID NO: 1575

SEQ ID NO: 1577 - RNA, 65 nt, Mus musculus
agcucugagaacugaauuccauggguuauaucaaugucagaccugugaaauucaguucuucagcu

SEQ ID NO: 1578 - RNA, 95 nt, Rattus norvegicus
uguguauccucagcucugagaacugaauuccauggguuauagcaaugucagaccugugaaguucaguucuuuagcugggauagcucuaucgucau

SEQ ID NO: 1579 - RNA, 99 nt, Gallus gallus
guguauccucagcuuugagaacugaauuccauggguuguaauugaauccuuugucagacccauggggcucaguucuucagcuuggauauuucugucuuc

SEQ ID NO: 1580 - RNA, 105 nt, Gallus gallus
ggacuggcugcuuggcuuugagaacugaauuccauaggcguuaaagcauccaaaaaugcccuauggauucaguucugcaguugggcagcaaauaaaccuccagcc

SEQ ID NO: 1581 - RNA, 90 nt, Xenopus tropicalis
caaugaguucuuagcuuugagaacugaauuccauagguuguuagagguuagaccuauggugcuuaguuccauagcuuggaucccacuggc

SEQ ID NO: 1582 - RNA, 114 nt, Danio rerio
gaguuuguuuugagcacuuuucccugagaacugaauuccauagaugguguucaugaaaaguucaucuaugggcucaguucuucuggcaaucuguuuaaugucugcuacaaauuc

SEQ ID NO: 1583 - RNA, 80 nt, Danio rerio
gcucuuggcuuugagaacugaauuccaagggugucugcuuuauauucagcccacggaguucaguucuuaaguuuggaugc

SEQ ID NO: 1584 - RNA, 80 nt, Danio rerio; identical to SEQ ID NO: 1583

SEQ ID NO: 1585 - RNA, 72 nt, Saguinus labiatus
aaucuaaagaaaacauuucugcacacacaccagacuauugaagccaguguguggaaaugcuucugccacauu

SEQ ID NO: 1586 - RNA, 72 nt, Macaca nemestrina
aaucuaaagaaaacauuucugcacacacaccagacuauugaagccaguguguggaaaugcuucugcuacauu

SEQ ID NO: 1587 - RNA, 72 nt, Pan paniscus
aaucuaaagaaaacauuucugcacacacaccagacuauggaagccaguguguggaaaugcuucugcuagauu

SEQ ID NO: 1588 - RNA, 72 nt, Pan troglodytes; identical to SEQ ID NO: 1587

SEQ ID NO: 1589 - RNA, 72 nt, Pongo pygmaeus; identical to SEQ ID NO: 1587

SEQ ID NO: 1590 - RNA, 66 nt, Mus musculus
ggcucuggcuccgugucuucacucccguguuuguccgaggagggagggagggacgggggcggugcu

SEQ ID NO: 1591 - RNA, 89 nt, Mus musculus
gccaaggguuugggggaacauucaaccugucggugaguuugggcagcucagacaaaccaucgaccguugaguggaccccgaggccugga

SEQ ID NO: 1592 - RNA, 106 nt, Rattus norvegicus
agaacuugccaaggguuugggggaacauucaaccugucggugaguuugggcagcucagacaaaccaucgaccguugaguggaccccgaggccuggaacugccaccc

SEQ ID NO: 1593 - RNA, 110 nt, Macaca mulatta
cggagaauuugccaaggguuugggggaacauucaaccugucggugaguuugggcagcucaggcaaaccaucgaccguugaguggacccugagaccuggacuugccauccu

SEQ ID NO: 1594 - RNA, 110 nt, Gorilla gorilla; identical to SEQ ID NO: 984

SEQ ID NO: 1595 - RNA, 110 nt, Pan paniscus; identical to SEQ ID NO: 984

SEQ ID NO: 1596 - RNA, 110 nt, Pan troglodytes; identical to SEQ ID NO: 984

SEQ ID NO: 1597 - RNA, 72 nt, Monodelphis domestica
ugggggaacauucaacgcugucggugaguuugagcagcugaaggcaaaccaucgaccguugaguggaccccg

SEQ ID NO: 1598 - RNA, 97 nt, Bos taurus
uugccaaggguuugggggaacauucaaccugucggugaguuugggcagcucaggcaaaccaucgaccguugaguggaccccgaggccuggaacugcc

SEQ ID NO: 1599 - RNA, 87 nt, Sus scrofa
caaggguuugggggaacauucaaccugucggugaguuugggcagcucaggcaaaccaucgaccguugaguggaccccgaggccugga

SEQ ID NO: 1600 - RNA, 78 nt, Danio rerio
ggguccugauucacauucauugcugucgguggguuuuaucucuucgacucgccggacaaugaaugagaacuacggcuc

SEQ ID NO: 1601 - RNA, 72 nt, Mus musculus
acaauuaacauucauuguugucgguggguugugaggaggcagccagacccaccgggggaugaaugucacugu

SEQ ID NO: 1602 - RNA, 83 nt, Rattus norvegicus
ggucacaauuaacauucauuguugucgguggguugugaggagaagaccagacccaccgggggaugaaugucacuguggcuggg

SEQ ID NO: 1603 - RNA, 105 nt, Xenopus tropicalis
aguggcuucucuggcaguguuuggcaaugguagaacucacacuggugagcuaugaagauccggugguucuagacuugccaacuauggccugggaauaaagcagca

SEQ ID NO: 1604 - RNA, 68 nt, Danio rerio
guauuuggcaaugguagaacucacacuggugagguagucagauccggugguucuagacuugccaacua

SEQ ID NO: 1605 - RNA, 70 nt, Mus musculus
cuguguauggcacugguagaauucacugugaacagucucagucagugaauuaccgaagggccauaaacag

SEQ ID NO: 1606 - RNA, 110 nt, Rattus norvegicus
ccagagagugugacuccuguccuguguauggcacugguagaauucacugugaacagucucggucagugaauuaccgaagggccauaaacagagcagagacagauccgcga

SEQ ID NO: 1607 - RNA, 110 nt, Saguinus labiatus
ccgcagaacgugacuccuguucuguguauggcacugguagaauucacugugaacagucucggucagugaauuaccgaagggccauaaacagagcagagacagauccucga

SEQ ID NO: 1608 - RNA, 110 nt, Macaca mulatta
ccgcagaacgugacuccuguucuguguauggcacugguagaauucacugugaacagucucggucagugaauuaccgaagggccauaaacagagcagagacagauccacga

SEQ ID NO: 1609 - RNA, 110 nt, Macaca nemestrina; identical to SEQ ID NO: 1608

SEQ ID NO: 1610 - RNA, 110 nt, Gorilla gorilla; identical to SEQ ID NO: 987

SEQ ID NO: 1611 - RNA, 110 nt, Pan paniscus; identical to SEQ ID NO: 987

SEQ ID NO: 1612 - RNA, 110 nt, Pan troglodytes; identical to SEQ ID NO: 987

SEQ ID NO: 1613 - RNA, 88 nt, Monodelphis domestica
acuccuguucuguguauggcacugguagaauucacugugaaaacacacuaucagugaauuaccaaagggccauaaacagagcagagaa

SEQ ID NO: 1614 - RNA, 98 nt, Gallus gallus
acuccuguucuguguauggcacugguagaauucacugugcaaccccgcggucagugaauuaccauagggccauaaacaaagcagagaaagacccgcga

SEQ ID NO: 1615 - RNA, 97 nt, Xenopus tropicalis
caucccuccuguucuguguauggcacugguagaauucacugugaaaacacaaaaucagugaauuaccauagggccauaaacagagcagagaaagaac

SEQ ID NO: 1616 - RNA, 70 nt, Sus scrofa
cuguguauggcacugguagaauucacugugaacagucucggucagugaauuaccgaagggccauaaacag

SEQ ID NO: 1617 - RNA, 98 nt, Danio rerio
gacuccuguucuguguauggcacugguagaauucacugugaaagcacacuaucagugaauuaccaaagggccauaaacagagcagagaaagaaccacg

SEQ ID NO: 1618 - RNA, 70 nt, Fugu rubripes
cuguguauggcacugguagaauucacugugagagcucacuaucagugaauuaccauagggccauaaacag

SEQ ID NO: 1619 - RNA, 70 nt, Tetraodon nigroviridis; identical to SEQ ID NO: 1618

SEQ ID NO: 1620 - RNA, 69 nt, Mus musculus
ccuuuccuuaucacuuuuccagccagcuuugugacucuaaguguuggacggagaacugauaaggguagg

SEQ ID NO: 1621 - RNA, 77 nt, Rattus norvegicus
cacuuucccuuaucaguuuuccagccagcuuugugacuguaaauguuggacggagaacugauaaggguaagugacug

SEQ ID NO: 1622 - RNA, 84 nt, Macaca nemestrina
ucagucacguccccuuaucacuuuuccagcccagcuuuaugacuguaaguguuggacggagaacugauaaggguaggugauuga

SEQ ID NO: 1623 - RNA, 84 nt, Pan troglodytes; identical to SEQ ID NO: 988

SEQ ID NO: 1624 - RNA, 84 nt, Pongo pygmaeus; identical to SEQ ID NO: 988

SEQ ID NO: 1625 - RNA, 84 nt, Monodelphis domestica
ccagucacauccccuuaucacuuuuccagcccagcuuucuaaugcuaauuguuggacggagaacugauaaggguaggugauuga

SEQ ID NO: 1626 - RNA, 74 nt, Gallus gallus
ccgcucucacccccuuaucacuuuuccagcccagcuucuucgcucugacuguuggacggagaacugauaagggu

SEQ ID NO: 1627 - RNA, 83 nt, Xenopus tropicalis
ccaguaucacuuccuuaucacuuuuccagcccagcuuuucaugacaaacuguuggacggagaacugauaaggcugugugacug

SEQ ID NO: 1628 - RNA, 83 nt, Sus scrofa
ccagucacauccccuuaucacuuuuccagccagcuuugugacucuaaauguuggacggagaacugauaaggguaggugauuga

SEQ ID NO: 1629 - RNA, 79 nt, Danio rerio
cacaucuccuuaucacuuuuccagcccagcuaacgauggugaauccguuggacggagaacugauaagggcauguggcug

SEQ ID NO: 1630 - RNA, 87 nt, Danio rerio
gucgaacacgucuccuuaucacuuuuccagcccagcuauccauuuaguauucguuggacggagaacugauaagggcaugugcccgau

SEQ ID NO: 1631 - RNA, 73 nt, Fugu rubripes
cgcucacaucuccuuaucacuuuuccagcccagcuauagguuuugaauccguuggacggagaacugauaaggg

SEQ ID NO: 1632 - RNA, 73 nt, Tetraodon nigroviridis
cgcucacaucuccuuaucacuuuuccagcccagcuauagcuucugaauccguuggacggagaacugauaaggg

SEQ ID NO: 1633 - RNA, 89 nt, Mus musculus
cgugcacagggcucugaccuaugaauugacagccaguacucuuuucucuccucuggcugccaauuccauaggucacagguauguucacc

SEQ ID NO: 1634 - RNA, 110 nt, Rattus norvegicus
gucaagauggagugcacagggcucugaccuaugaauugacagccaguacucugaucucgccucuggcugccaguuccauaggucacagguauguucgccucaaugccagc

SEQ ID NO: 1635 - RNA, 87 nt, Xenopus tropicalis
gaguguacgggccuaugaccuaugaauugacagccaguggaugugaagucugccugucaauucuguaggccacagguucguccaccu

SEQ ID NO: 1636 - RNA, 106 nt, Bos taurus
agaccgagugcacagggcucugaccuaugaauugacagccagugcucuuguguccccucuggcugccaauuccauaggucacagguauguucgccucaaugccagc

SEQ ID NO: 1637 - RNA, 85 nt, Danio rerio
cuaggacacagggugaugaccuaugaauugacagccaguguuugcaguccagcugccugucaguucuguaggccacugcccuguu

SEQ ID NO: 1638 - RNA, 82 nt, Fugu rubripes
ugggacgugaggugaugaccuaugaauugacagccaguaacuggagccucugccugucaguucuguaggccacugcuacguu

SEQ ID NO: 1639 - RNA, 83 nt, Tetraodon nigroviridis
cacgaggugaugaccuaugaauugacagccaguaacuggagccucugccugucaguucuguaggccacugcugcguccguccc

SEQ ID NO: 1640 - RNA, 112 nt, Mus musculus
agcucucagcaucaacgguguacaggagaaugaccuaugauuugacagaccgugcagcuguguaugucugucauucuguaggccaauauucuguaugucacugcuacuuaaa

SEQ ID NO: 1641 - RNA, 97 nt, Rattus norvegicus
gguguacaggacaaugaccuaugauuugacagacaguguggcugcgugugucugucauucuguaggccaauauucuguaugucucuccuccuuacaa

SEQ ID NO: 1642 - RNA, 110 nt, Macaca mulatta
aucauuaagaaaugguauacaggaaaaugaccuaugaauugacagacacuauagcugaguuugucugucauuucuuuaggccaauauucuguaugacugugcuacuucaa

SEQ ID NO: 1643 - RNA, 110 nt, Macaca nemestrina; identical to SEQ ID NO: 1642

SEQ ID NO: 1644 - RNA, 110 nt, Gorilla gorilla
aucauucagaaaugguauacgggaaaaugaccuaugaauugacagacaauauagcugaguuugucugucauuucuuuagaccaauauucuguaugacugugcuacuucaa

SEQ ID NO: 1645 - RNA, 110 nt, Pan troglodytes
aucauucagaaaugguauacgggaaaaugaccuaugaauugacagacaauauagcugaguuugucugucauuucuuuaggccaauauucuguaugacugugcuacuucaa

SEQ ID NO: 1646 - RNA, 110 nt, Pongo pygmaeus
aucauucagaaaugguauacaggaaaaugaccuaugaauugacagacaauacagcugaguuugucugucauuucuuuaggccaauauucuguacaacugugcuacuucaa

SEQ ID NO: 1647 - RNA, 105 nt, Gallus gallus
ucaguaagaacugguguccaggaaaaugaccuaugaauugacagacugcuuucaaaaugugccugucauuucuauaggccaauauucugugcacuuuuccuacuu

SEQ ID NO: 1648 - RNA, 97 nt, Xenopus tropicalis
aacugguaaccaggaggaugaccuaugaaaugacagccacuuccauaccaaacaugucugucauuucuguaggccaauauucugauugcuuuguuga

SEQ ID NO: 1649 - RNA, 79 nt, Bos taurus
uguacaggaaaaugaccuaugaauugacagacaacgugacuaagucugucugucauuucuguaggccaauguucuguau

SEQ ID NO: 1650 - RNA, 86 nt, Rattus norvegicus
gcggacgggagcugagagcugggucuuugcgggcaagaugagggugucaguucaacuggccuacaaagucccaguccucggcuccc

SEQ ID NO: 1651 - RNA, 66 nt, Monodelphis domestica
gaggauugggucuuugugggcgagaugagggugucaguucaacuggccuacaaagucccaguucuc

SEQ ID NO: 1652 - RNA, 83 nt, Gallus gallus
gugguuccagagucgggguuuugggggcgagaugagcuuauguuuuauccaacuggcccacaaagucccgcuuuuggugguca

SEQ ID NO: 1653 - RNA, 83 nt, Xenopus tropicalis
gugguuucggagucgggauuuugggggcgagaugagcuaaugauuuauccaacuggcccgcaaagucccgcuucuggaaguca

SEQ ID NO: 1654 - RNA, 81 nt, Bos taurus
ugggagcugagagcugggucuuugcgggcgagaugaaggugucgguucaacuggccuacaaagucccaguccucggccccc

SEQ ID NO: 1655 - RNA, 99 nt, Danio rerio
gacauuaauguguuagagguugggucuuugcgggcaaggugaguaguuaaauuuacucucaacuggccuacaaagucccaguuucuggcucauguuauc

SEQ ID NO: 1656 - RNA, 98 nt, Danio rerio
gaaagugugucagaggcugggucuuugcgggcaaggugaguuuuccuuuuauucaacuggccuacaaagucccaguuuucggcccaugugaccaucuc

SEQ ID NO: 1657 - RNA, 98 nt, Danio rerio; identical to SEQ ID NO: 1656

SEQ ID NO: 1658 - RNA, 88 nt, Fugu rubripes
ugugucagaagcugggucuuugcgggcaaggugaguccucaguuuguucaacuggccuacaaagucccagucucuggcucacguaacc

SEQ ID NO: 1659 - RNA, 83 nt, Tetraodon nigroviridis
cagaagcugggucuuugcgggcaaggugaguccucaguuuguucaacuggccuacaaagucccagugucuggcucacgugacc

SEQ ID NO: 1660 - RNA, 79 nt, Mus musculus
ggcccagaaucgggguuuugagggcgagaugaguuuguguuuuauccaacuggcccacaaagucccgcuuuugggguca

SEQ ID NO: 1661 - RNA, 85 nt, Danio rerio
gugauuucagugacgggacuuugggggcgagaugaguauugaucucuauccaacuggcccgcaaagucccgcuucugggacucac

SEQ ID NO: 1662 - RNA, 94 nt, Mus musculus
acacccaacucuccuggcucuagcagcacagaaauauuggcauggggaagugagucugccaauauuggcugugcugcuccaggcagggugguga

SEQ ID NO: 1663 - RNA, 87 nt, Rattus norvegicus
aacucuccuggcucuagcagcacagaaauauuggcacggguaagugagucugccaauauuggcugugcugcuccaggcaggguggug

SEQ ID NO: 1664 - RNA, 87 nt, Gorilla gorilla
agcuuccugggcucuagcagcacagaaauauuggcacagggaagcgagucugccaauauuggcugugcugcuccaggcaggguggug

SEQ ID NO: 1665 - RNA, 87 nt, Pan paniscus; identical to SEQ ID NO: 993

SEQ ID NO: 1666 - RNA, 87 nt, Bos taurus
agcuccccuggcucuagcagcacagaaauauuggcacugggaagaaagccugccaauauuggcugugcugcuccaggcaggguggug

SEQ ID NO: 1667 - RNA, 84 nt, Mus musculus
ccugcccccgccccagcagcacacugugguuuguacggcacuguggccacguccaaaccacacugugguguuagagcgagggua

SEQ ID NO: 1668 - RNA, 69 nt, Rattus norvegicus
ccagcagcacacugugguuuguacggcacuguggccacguccaaaccacacugugguguuagagcgagg

SEQ ID NO: 1669 - RNA, 112 nt, Bos taurus
ccaccccaguccugcucccgccccagcagcacacugugguuuguacggcacuguggccacguccaaaccacacugugguguuagagcgagggugggggaggcaccgcugagg

SEQ ID NO: 1670 - RNA, 102 nt, Mus musculus
ugagccgggacuguugagugaaguagguaguuucauguuguugggccuggcuuucugaacacaacgacaucaaaccaccugauucauggcaguuacugcuuc

SEQ ID NO: 1671 - RNA, 85 nt, Mus musculus
agcugaucuguggcuuagguaguuucauguuguugggauugaguuuugaacucggcaacaagaaacugccugaguuacaucaguc

SEQ ID NO: 1672 - RNA, 110 nt, Rattus norvegicus
uguuugcucagcugaucuguggcuuagguaguuucauguuguugggauugaguuuugaacucggcaacaagaaacugccugaguuacaucagucgguuuucgucgagggc

SEQ ID NO: 1673 - RNA, 110 nt, Ateles geoffroyi
ugcuugcucagcugaucuguggcuuagguaguuucauguuguugggauugaguuuugaacucggcaacaagaaacugccugaguuacaucagucgguuuucgucgagggg

SEQ ID NO: 1674 - RNA, 110 nt, Lagothrix lagotricha
ugcuugcucagcugaucuguggcuuagguaguuucauguuguugggauugaguuuugaacucggcaacaagaaacugccugaguuacaucagucgguuuucgucgagggc

SEQ ID NO: 1675 - RNA, 70 nt, Macaca mulatta; identical to SEQ ID NO: 996

SEQ ID NO: 1676 - RNA, 70 nt, Gorilla gorilla; identical to SEQ ID NO: 996

SEQ ID NO: 1677 - RNA, 110 nt, Gorilla gorilla; identical to SEQ ID NO: 997

SEQ ID NO: 1678 - RNA, 110 nt, Pan paniscus; identical to SEQ ID NO: 997

SEQ ID NO: 1679 - RNA, 110 nt, Pan troglodytes; identical to SEQ ID NO: 997

SEQ ID NO: 1680 - RNA, 70 nt, Pongo pygmaeus; identical to SEQ ID NO: 996

SEQ ID NO: 1681 - RNA, 110 nt, Pongo pygmaeus; identical to SEQ ID NO: 997

SEQ ID NO: 1682 - RNA, 95 nt, Gallus gallus
ugaacuagaacugcucugugaauuagguaguuucauguuguugggcuuuaaauuuuaaacacaagaacaucaaacuaccugauuuacuccaguua

SEQ ID NO: 1683 - RNA, 94 nt, Gallus gallus
aacugcucugugguuuagguaguuucauguuguuggggcuccaccuuucucucuacagcacgaaacugccuuaauuacuucaguugauaucauc

SEQ ID NO: 1684 - RNA, 108 nt, Gallus gallus
uuucaugcagcugaucugugguuuagguaguuucauguuguugggauuggcuuuuagcucggcaacaagaaacugccuuaauuacgucaguuagucuucaucaagggc

SEQ ID NO: 1685 - RNA, 106 nt, Xenopus tropicalis
uuuguauccagcugaucugugguuuagguaguuucauguuguugggauugcuuuuucuuaacgcggcaacaagaaacugccuuaauuacgucaguucgucuucauc

SEQ ID NO: 1686 - RNA, 107 nt, Sus scrofa
uugcucagcugaucuguggcuuagguaguuucauguuguugggauugaguuuugaacucggcaacaagaaacugccugaguuacaucagucgguuaucgucgagggc

SEQ ID NO: 1687 - RNA, 106 nt, Danio rerio
cgcgcggcuggugcgugguuuagguaguuucauguuguugggauuggcuuccuggcucgacaacaagaaacugccuugauuacgucaguucgucuucaucaagggc

SEQ ID NO: 1688 - RNA, 76 nt, Danio rerio
ucgagugguuuagguaguuucauguuguugggauuacauucaaacucugcaacgugaaacugucuuaauugcccca

SEQ ID NO: 1689 - RNA, 85 nt, Fugu rubripes
agcuggagcgugguuuagguaguuucauguuguuggggauggcuuccuggcucggcaacaagaaacugccuugauuacgucaguu

SEQ ID NO: 1690 - RNA, 76 nt, Fugu rubripes
ucgagugguuuagguaguuucauguuguugggguccauuucaaacucugcaacaugaaacugucuuaauugcccca

SEQ ID NO: 1691 - RNA, 101 nt, Tetraodon nigroviridis
agcuggagcgugguuuagguaguuucauguuguuggggauggcuuccuggcucggcaacaagaaacugccuugauuacgucaguucgucuucaucaagggc

SEQ ID NO: 1692 - RNA, 76 nt, Tetraodon nigroviridis; identical to SEQ ID NO: 1690

SEQ ID NO: 1693 - RNA, 85 nt, Mus musculus
aacuggucggugauuuagguaguuuccuguuguugggauccaccuuucucucgacagcacgacacugccuucauuacuucaguug

SEQ ID NO: 1694 - RNA, 85 nt, Rattus norvegicus; identical to SEQ ID NO: 1693

SEQ ID NO: 1695 - RNA, 86 nt, Monodelphis domestica
aacuggucugugauuuagguaguuuccuguuguuggggcuccaccuuucucucgacagcacgauacugccuucauuacuucaguug

SEQ ID NO: 1696 - RNA, 87 nt, Xenopus tropicalis
agccgcugugugguuuagguaguuuuauguuguugggcauucaccuuucucucuacaacaagaaacugccuuaauuacaucaguggg

SEQ ID NO: 1697 - RNA, 79 nt, Danio rerio
gucugugauuuagguaguuucaaguuguugggcuggacguuuaauuucacaggaaccugaaacugccugaauugcucca

SEQ ID NO: 1698 - RNA, 69 nt, Fugu rubripes
ugugauauagguaguuucaaguuguugggcugaacucuugugaucacaggaaccugaaacugccugagu

SEQ ID NO: 1699 - RNA, 69 nt, Tetraodon nigroviridis
ugugauguagguaguuucaaguuguugggcuaaacuuuggugaucacaggaaccugaaacugcccgauu

SEQ ID NO: 1700 - RNA, 81 nt, Mus musculus
ggggcugugccggguagagagggcagugggagguaagagcucuucacccuucaccaccuucuccacccagcauggccggca

SEQ ID NO: 1701 - RNA, 75 nt, Ateles geoffroyi; identical to SEQ ID NO: 999

SEQ ID NO: 1702 - RNA, 75 nt, Macaca nemestrina
ggcugugccggguagagagggcagugggagguaagagcucuucacccuucaccaccuucuccacccggcauggcc

SEQ ID NO: 1703 - RNA, 75 nt, Pan paniscus; identical to SEQ ID NO: 999

SEQ ID NO: 1704 - RNA, 75 nt, Pan troglodytes; identical to SEQ ID NO: 999

SEQ ID NO: 1705 - RNA, 75 nt, Pongo pygmaeus; identical to SEQ ID NO: 999

SEQ ID NO: 1706 - RNA, 96 nt, Xenopus tropicalis
aagaaccugcuccgucgccccaguguucagacuaccuguucaggacaaugcuguuguacaguagucugcacauugguuagacugggcaugggacag

SEQ ID NO: 1707 - RNA, 87 nt, Danio rerio
uccugcuccgucaucccaguguucagacuaccuguucaggaucauacugguguacaguagucugcacauugguuagacugugcaugg

SEQ ID NO: 1708 - RNA, 96 nt, Danio rerio
ggaguuuuuguggacgcccgucccgccugcccaguguucagacuaccuguucaggaauuaguguuuguacaguagucugcacauugguuaggcugg

SEQ ID NO: 1709 - RNA, 98 nt, Danio rerio
ccucccccucgccugcccaguguucagacuaccuguucaucaugcugcagcugaacaguaguccgcacauugguuaggcugggcugggacacacacac

SEQ ID NO: 1710 - RNA, 90 nt, Mus musculus
cugggccucugugggcaucuuaccggacagugcuggauuucuuggcuugacucuaacacugucugguaacgauguucaaaggugacccac

SEQ ID NO: 1711 - RNA, 89 nt, Rattus norvegicus
cugggccucugugggcaucuuaccggacagugcuggauuucuuggcuugacucuaacacugucugguaacgauguucaaaggugaccca

SEQ ID NO: 1712 - RNA, 86 nt, Monodelphis domestica
gggccucugugggcaucuuacuagacagugcuggauuuuuggauguacucuaacacugucugguaacgauguuuaaagagggaacc

SEQ ID NO: 1713 - RNA, 87 nt, Gallus gallus
gguccucugugggcaucuuacuagacagugcuggauuucuuggaucuauucuaacacugucugguaacgauguuuaaagggugaacc

SEQ ID NO: 1714 - RNA, 89 nt, Xenopus tropicalis
ugguccucuauggacaucuuacuagacagugcuggauuuauuuuaucuuuucuaacacugucugguaacgauguuuaaagagugagcca

SEQ ID NO: 1715 - RNA, 86 nt, Bos taurus
gggccucuguggacaucuuaccggacagugcuggauuucucggcucgacucuaacacugucugguaacgauguucaaaggugaccc

SEQ ID NO: 1716 - RNA, 86 nt, Danio rerio
ggcacuuagcagccaucuuaccggacagugcuggacuguauaacuguuuucuaacacugucugguaacgauguuuguugggugacc

SEQ ID NO: 1717 - RNA, 83 nt, Fugu rubripes
ucucaggauccaucuuacccgacagugcuggauuguacuacuguuguucuaacacugucugguaacgauguuuucugggugac

SEQ ID NO: 1718 - RNA, 62 nt, Tetraodon nigroviridis
caucuuaccugacagugcuggauuauacuacuguuguucuaacacugucugguaacgauguu

SEQ ID NO: 1719 - RNA, 68 nt, Mus musculus
uggacuucccuuugucauccuaugccugagaauauaugaaggaggcugggaaggcaaagggacguuca

SEQ ID NO: 1720 - RNA, 110 nt, Rattus norvegicus
ggcuacagcccuucuucaugugacucguggacuucccuuugucauccuaugccugagaauauaugaaggaggcugggaaggcaaagggacguucaauugucaucacuggc

SEQ ID NO: 1721 - RNA, 110 nt, Saguinus labiatus; identical to SEQ ID NO: 1004

SEQ ID NO: 1722 - RNA, 110 nt, Macaca nemestrina; identical to SEQ ID NO: 1004

SEQ ID NO: 1723 - RNA, 110 nt, Gorilla gorilla; identical to SEQ ID NO: 1004

SEQ ID NO: 1724 - RNA, 110 nt, Pan paniscus; identical to SEQ ID NO: 1004

SEQ ID NO: 1725 - RNA, 110 nt, Pan troglodytes; identical to SEQ ID NO: 1004

SEQ ID NO: 1726 - RNA, 110 nt, Pongo pygmaeus; identical to SEQ ID NO: 1004

SEQ ID NO: 1727 - RNA, 97 nt, Monodelphis domestica
caacugacuaugugaccugugggcuucccuuugucauccuaugccuggaaaucagaguggggcagggacagcaaagggaugcucagcugucgucuuc

SEQ ID NO: 1728 - RNA, 104 nt, Gallus gallus
gucaacagugucuguucaugugacccguggacuucccuuugucauccuaugccugaaaauauaugaagggggcggggaaggcaaagggacguucaacugucauc

SEQ ID NO: 1729 - RNA, 101 nt, Gallus gallus
cuacugagacugugugaccugugggcuucccuuugucauccuaugccuggagaucacagugaggcagggacagcaaagggaugcucagcugucgucucuuc

SEQ ID NO: 1730 - RNA, 105 nt, Xenopus tropicalis
uagaggacuucgacauugugacccaugggcuucccuuugucauccuaugccugagaaugcuggagaggcagggacagcaaagggaugcucagauguuaccucuug

SEQ ID NO: 1731 - RNA, 88 nt, Xenopus tropicalis
augugaccugugggcuucccuuugucauccuaugccugagaauauaugaagggggcugggaaggcaaagggacguucaguugucauca

SEQ ID NO: 1732 - RNA, 96 nt, Bos taurus
uuucuucaugugacucguggacuucccuuugucauccuaugccugagaauauaugaaggaggcugggaaggcaaagggacguucaauugucaucac

SEQ ID NO: 1733 - RNA, 109 nt, Sus scrofa
ggcuacaguccuucucaugugacucguggacuucccuuugucauccuaugccugagaauauaugaaggaggcugggaaggcaaagggacguucaauugucaucacuggc

SEQ ID NO: 1734 - RNA, 93 nt, Danio rerio
ucaugugaccuguggacuucccuuugucauccuaugccuggaguaauagagggggcugggaagucaaagggacgcucaggcgucaucauucgc

SEQ ID NO: 1735 - RNA, 124 nt, Danio rerio
guguugcucuagugaccaguuugugaccuccuggguuucccuuugucauccuaugccugcaguuccugaugaggcugggacagcaaagggagguucagaugucgaccuguacuacagucaauac

SEQ ID NO: 1736 - RNA, 124 nt, Danio rerio; identical to SEQ ID NO: 1735

SEQ ID NO: 1737 - RNA, 92 nt, Fugu rubripes
ugugaccugugggcuucccuuugucauccuaugccuggagcucggauaaggcagggacagcaaagggaugcucagcugucaccacugacauc

SEQ ID NO: 1738 - RNA, 93 nt, Tetraodon nigroviridis
ugugaccugugggcuucccuuugucauccuaugccuggagcucggauaaggcagggacagcaaagggaugcucagccgucaccacugacuuca

SEQ ID NO: 1739 - RNA, 106 nt, Mus musculus
cugcuuggaccugugaccugugggcuucccuuugucauccuuugccuaggccucugagugaggcaaggacagcaaaggggggcucaguggucaccucuacugcaga

SEQ ID NO: 1740 - RNA, 106 nt, Rattus norvegicus
cagcuuggaccugugaccucugggcuucccuuugucauccuuugccuaggccucugaguggggcaaggacagcaaaggggggcucaguggucaccucuacugcaga

SEQ ID NO: 1741 - RNA, 109 nt, Macaca mulatta
ucacuggccaugugacuugugggcuucccuuugucauccuuugccuagggcucugagcagggcagggacagcaaaggggugcucaguugucacuucccacagcacagag

SEQ ID NO: 1742 - RNA, 109 nt, Macaca nemestrina
ucacuggccaugugacuugugggcuucccuuugucauccuucgccuagggcucugagcagggcagggacagcaaaggggugcucaguugucacuucccacagcacagag

SEQ ID NO: 1743 - RNA, 110 nt, Pongo pygmaeus
ucacccagccaugugacuuguggacuucccuuugucauccuucgccuagggcucugagcagggcagggacagcaaaggggugcucaguugucacuucccacagcacggag

SEQ ID NO: 1744 - RNA, 74 nt, Gallus gallus
ccugugagcuucccuuugucauccuaugccugagcgaugccuggaggcugggacggugaagggaggcccacggg

SEQ ID NO: 1745 - RNA, 68 nt, Mus musculus
cucuuguccuucauuccaccggagucugucuuaugccaaccagauuucaguggagugaagcucaggag

SEQ ID NO: 1746 - RNA, 108 nt, Rattus norvegicus
aaacagccccagacaauccauggguccuccuguccuucauuccaccggagucugucuuaugccaaccagauuucaguggagugaagcucaggaggcauggagcugcca

SEQ ID NO: 1747 - RNA, 109 nt, Ateles geoffroyi
aaagauccucgggcaauccaugugcuucucuuguccuucauuccaccggagucugucucauacccaaccagauuucaguggagugaagucaggaggcauggagcugacg

SEQ ID NO: 1748 - RNA, 110 nt, Lagothrix lagotricha
aaagauccucgggcaauccaugugcuucucuuguccuucauuccaccggagucugucucauacccaaccagauuucaguggagugaaguucaggaggcauggagcugacg

SEQ ID NO: 1749 - RNA, 110 nt, Macaca nemestrina
aaagauccucaggcaauccaugugcuucucuuguccuucauuccaccggagucugucucauacccaaccagauuucaguggagugaaguucaggaggcauggagcugacg

SEQ ID NO: 1750 - RNA, 110 nt, Gorilla gorilla; identical to SEQ ID NO: 1006

SEQ ID NO: 1751 - RNA, 110 nt, Pan paniscus; identical to SEQ ID NO: 1006

SEQ ID NO: 1752 - RNA, 110 nt, Pan troglodytes; identical to SEQ ID NO: 1006

SEQ ID NO: 1753 - RNA, 96 nt, Gallus gallus
gacaauccauggguucuguuguccuucauuccaccggagucugucucguaccuaaccagauuucaguggagugaagcacaagagacauggagauga

SEQ ID NO: 1754 - RNA, 98 nt, Gallus gallus
auacacuacuagggccuuccugaugcccuucauuccaccggaaucugucagugcagaaaccagauuucagugaaaugaagcccaucagagaggcagcu

SEQ ID NO: 1755 - RNA, 86 nt, Xenopus tropicalis
ccacguguccugcuguccuucauuccaccggagucugucucauacauaaucagauuucaguggagugaagcacaagaggcauguag

SEQ ID NO: 1756 - RNA, 69 nt, Bos taurus
cucuuguccuucauuccaccggagucugucucguacccaaccagauuucaguggagugaaguucaggag

SEQ ID NO: 1757 - RNA, 69 nt, Sus scrofa
cucuuguccuucauuccaccggagucugucucauacccaaccagauuucaguggagugaagcucaggag

SEQ ID NO: 1758 - RNA, 89 nt, Danio rerio
aaacuacugugcauucuauccuucauuccaccggagucuguguaguuguucaaucagauuucaguggugugaaguguaggaaacacgga

SEQ ID NO: 1759 - RNA, 79 nt, Fugu rubripes
uguguucuauccuucauuccaccggagucuguauguguauuuaaccagauuucaguggugugaaguguaagagacaugg

SEQ ID NO: 1760 - RNA, 66 nt, Tetraodon nigroviridis
uauccuucauuccaccggagucuguaacuguuuuuaaccagauuucaguggugugaaguguaagag

SEQ ID NO: 1761 - RNA, 83 nt, Mus musculus
uuccuuugacgggugagcuuuuggcccggguuauaccugacacucacguauaagacgagcaaaaagcuuguuggucagaggag

SEQ ID NO: 1762 - RNA, 83 nt, Rattus norvegicus
uuccuuugacgggugagcuuuuggcccggguuauaccugacucucacguauaagacgagcaaaaagcuuguuggucagaggag

SEQ ID NO: 1763 - RNA, 77 nt, Monodelphis domestica
uuccugugacaggugagcuuuugguccggguuauaccugaugcauguguauaagacgagcaaaaagcucguuggucg

SEQ ID NO: 1764 - RNA, 81 nt, Xenopus tropicalis
uccuuucaacaggcaagcuuuuugcuuggauuauguuuucuguuguuguauaagacgagcauaaagcuuguuuguuagaag

SEQ ID NO: 1765 - RNA, 79 nt, Mus musculus
cccucaguggcucaguagccaguguagauccugucuuugguaaucagcagcuacaucuggcuacugggucucugguggc

SEQ ID NO: 1766 - RNA, 103 nt, Rattus norvegicus
aaggauuagggugcccucaguggcucaguagccaguguagauccugucuuugguaaucagcagcuacaucuggcuacugggucucugauggcaucaucuagcu

SEQ ID NO: 1767 - RNA, 110 nt, Ateles geoffroyi
ucugccggaagguguggguacccucaguggcucaguagccaguguagauccugucuuuuguaaucaguagcuacaucuggcuacugggucucugauggcaucuuuuagcu

SEQ ID NO: 1768 - RNA, 98 nt, Gallus gallus
uguaguugcccaucaaucgcucaguagucaguguagauucugucuuuacaaucagcagcuacaucuggcuacugggucucugaugacaucucauaucu

SEQ ID NO: 1769 - RNA, 64 nt, Xenopus tropicalis
cgcucaguaaucaguguagauccuguauaucugucagcagcuacaucuggcuacugggucucua

SEQ ID NO: 1770 - RNA, 110 nt, Bos taurus
gcugcuggaauguguagguacccucaauggcucaguagccaguguagauccugucuuuuguaaucaguagcuacaucuggcuacugggucucugauggcaucuucuaccu

SEQ ID NO: 1771 - RNA, 108 nt, Danio rerio
gccgguggcuuguucgggugcucaugagaugcucaguagucaguguagauccugugucacaaucagcagcuacaucuggcuacugggucucugauggcauuuucugcu

SEQ ID NO: 1772 - RNA, 69 nt, Fugu rubripes
ucaguugcucaguaggcaguguagauccuguguagcaaucagcagcuacaucuggcuacugggucucug

SEQ ID NO: 1773 - RNA, 68 nt, Tetraodon nigroviridis
caguugcucaguaggcaguguagauccuguguagcaaucagcagcuacaucuggcuacugggucucug

SEQ ID NO: 1774 - RNA, 89 nt, Mus musculus
aacugacuaugccuccucgcauccccuagggcauugguguaaagcuggagacccacugccccaggugcugcuggggguuguagucugac

SEQ ID NO: 1775 - RNA, 83 nt, Rattus norvegicus
cugacuaugccuccucgcauccccuagggcauugguguaaagcuggagacccacugccccaggugcugcuggggguuguaguc

SEQ ID NO: 1776 - RNA, 97 nt, Mus musculus
uguucgcuucugguaccggaagagagguuuucugggucucuguuucuuugaugagaaugaaacacacccagcuaaccuuuuuuucaguaucaaaucc

SEQ ID NO: 1777 - RNA, 97 nt, Rattus norvegicus; identical to SEQ ID NO: 1776

SEQ ID NO: 1778 - RNA, 96 nt, Rattus norvegicus
gagucuggucuuguuuggguuuguucuagguauggucccagggaucccagaucaaaccaggccccugggccuauccuagaaccaaccuaaacccau

SEQ ID NO: 1779 - RNA, 90 nt, Bos taurus
gaguuugguuuuguuuggguuuguucuagguauggucccagggaucccagaucaaaccaggccccugggccuauccuagaaccaaccuaa

SEQ ID NO: 1780 - RNA, 94 nt, Bos taurus
uggaagcgggugcgaggcgaggggugcuaucugugguugaggacacggcaaaugaaacugucucacacagaaaucgcacccaucuccucggccc

SEQ ID NO: 1781 - RNA, 98 nt, Mus musculus
ucuguguugggcgucugucugcccgagugccugccucucuguugcucugaaggaggcaggggcugggccugcagcugccugggcagagcugcuccuuc

SEQ ID NO: 1782 - RNA, 98 nt, Rattus norvegicus
ucuguguugggcaucugucugccugagugccugccucucuguugcucugaaggaggcaggggcugggccugcagcugccugggcagagcugcuccuuc

SEQ ID NO: 1783 - RNA, 82 nt, Mus musculus
ugguauuuaaaagguggauauuccuucuaugguuacgugcuuccuggauaaucauagaggaacauccacuuuuucaguauca

SEQ ID NO: 1784 - RNA, 68 nt, Mus musculus
ugagcagagguugcccuuggugaauucgcuuuauugauguugaaucacacaaaggcaacuuuuguuug

SEQ ID NO: 1785 - RNA, 72 nt, Rattus norvegicus
cuugagcagagguugcccuuggugaauucgcuuuauugauguugaaucacacaaaggcaacuuuugguugag

SEQ ID NO: 1786 - RNA, 61 nt, Bos taurus
aagaugguugaccauagaacaugcgcugucucuaugucguauguaaugugguccacgucuu

SEQ ID NO: 1787 - RNA, 76 nt, Mus musculus
uacuugaagagaaguuguucgugguggauucgcuuuacuugugacgaaucauucacggacaacacuuuuuucagua

SEQ ID NO: 1788 - RNA, 80 nt, Rattus norvegicus
gguacuugaagagaaguuguucgugguggauucgcuuuacuugugacgaaucauucacggacaacacuuuuuucaguacc

SEQ ID NO: 1789 - RNA, 70 nt, Rattus norvegicus
gggacggauggucgaccagcuggaaaguaauuguuucuaauguacuucaccugguccacuagccgucggu

SEQ ID NO: 1790 - RNA, 79 nt, Rattus norvegicus
gaaguuaggcugaggggcagagagcgagacuuuucuauuuuccaaaagcucggucugaggccccucagucuugcuuccu

SEQ ID NO: 1791 - RNA, 91 nt, Mus musculus
cguccugcgaggugucuugcaggccgucaugcaggccacacugacgguaacguugcaggucgucuugcagggcuucucgcaagacgacauc

SEQ ID NO: 1792 - RNA, 114 nt, Rattus norvegicus
uccugcgcguccugcgaggugucuugcaggccgucaugcaggccacacugacgguaacguugcaggucgucuugcagggcuucucgcaagacgacaucuucaucgccaacgacg

SEQ ID NO: 1793 - RNA, 91 nt, Ovis aries
cguccugcgaggugucuugcaggccgucaugcaggccacacugacggaaacguugcaggucgucuugcagggcgucucgcaagacgacaca

SEQ ID NO: 1794 - RNA, 124 nt, Mus musculus
ugcccggggagaaguacggugagccugucauuauucagagaggcuagauccucuguguugagaaggaucaugaugggcuccucgguguucuccagguagcggcaccacaccaugaaggcagccc

SEQ ID NO: 1795 - RNA, 93 nt, Rattus norvegicus; identical to SEQ ID NO: 1035

SEQ ID NO: 1796 - RNA, 80 nt, Mus musculus
agacucggguaggcaguguuguuagcuggcugcguugggucaggccagcagccacagcuacccugccacuuccuucuggc

SEQ ID NO: 1797 - RNA, 109 nt, Mus musculus
aggaugaagugugggugugucaggcaggcagugcauugcuagcuggcuguuagaacuuuaucccaacaguugcuagcugcacuacccucugcugcacucagaagcaugc

SEQ ID NO: 1798 - RNA, 91 nt, Rattus norvegicus
cugugugcgauggguuggcaguguauuguuagcugguugaguauguaaaaggcaccagcuaacaugcaacugcucuccuauugcacauaca

SEQ ID NO: 1799 - RNA, 91 nt, Canis familiaris
ccgugugugauggguuggcaguguauuguuagcugguugaauauaugaauggcaucagcuaacaugcaacugcuaucuuauugcauauaca

SEQ ID NO: 1800 - RNA, 79 nt, Monodelphis domestica
ugugaugggauggcaguguauuguuagcugguugacuaucugaacgugccagcuaacaugcaacugcuaucccauugca

SEQ ID NO: 1801 - RNA, 86 nt, Gallus gallus
cugugugcggugggguggcaguguauguuagcugguugaaacucuugacaucagcuaacacgcaguugcuaaccugcuccacauac

SEQ ID NO: 1802 - RNA, 87 nt, Xenopus tropicalis
cuguguuuagaaggaaggcaguguaauguuagcugguuggaaaauagcagacacugguuaacuuacaccugccccccuucuuuacau

SEQ ID NO: 1803 - RNA, 72 nt, Mus musculus
cuugggaauggcgaggaaaccguuaccauuacugaguuuaguaaugguaacgguucucuugcugcucccaca

SEQ ID NO: 1804 - RNA, 72 nt, Rattus norvegicus
uuugggaauggcgaggaaaccguuaccauuacugaguuuaguaaugguaaugguucucuugcugcucccaca

SEQ ID NO: 1805 - RNA, 62 nt, Monodelphis domestica
cuggcggggaaaccguuaccauuacuguguuuaguaaugguaaggguucucccgcugcgcug

SEQ ID NO: 1806 - RNA, 69 nt, Gallus gallus
acacauggcuggcagggaaaccguuaccauuacugaguuuaguaaugguaacgguucugcugacagcca

SEQ ID NO: 1807 - RNA, 62 nt, Xenopus tropicalis
gaguggcaaugaaaccguuaccauuacugaguuuaguaaugguaaggguucuguugcugcuc

SEQ ID NO: 1808 - RNA, 60 nt, Danio rerio
agaggcggcgaaaccguuaccauuacugaguuuaguaaugguaaggguucugcugccuuu

SEQ ID NO: 1809 - RNA, 85 nt, Mus musculus
gcuaagcaguuacaacuguuugcagaggaaacugagacuuuauaacuaugucucagucucaucugcaaagagguaagugcuuugc

SEQ ID NO: 1810 - RNA, 78 nt, Rattus norvegicus
ggugugagcguaugugccuuuggacuacaucguggacgcagcaccaugcaguccacgggcauauacacuugccucaag

SEQ ID NO: 1811 - RNA, 86 nt, Gallus gallus
ucccuggugugaggguaugugcccuuggacuacaucguggaagccagcaccaugcaguccaugggcauauacacuugccucaaggu

SEQ ID NO: 1812 - RNA, 87 nt, Xenopus tropicalis
ucccuggcgugaggguaugugcccuuggacuacaucguggaagccagcaccaugcaguccaugggcauauacacuugccucaagguu

SEQ ID NO: 1813 - RNA, 89 nt, Bos taurus
ucccuggcgugaggguaugugccuuuggacuacaucguggaagccagcaccaugcaguccaugggcauauacacuugccucaaggccua

SEQ ID NO: 1814 - RNA, 96 nt, Danio rerio
ucccuggugugaggguaugugcccuuggacuacaucguggaagccagcaccaugcaguccaugggcauauacacuugccucaaggccuagaucuuc

SEQ ID NO: 1815 - RNA, 84 nt, Fugu rubripes
cccuggugugaggguaugugcccuuggacuacaucguggaagccagcaccaugcaguccaugggcauauacacuugccucaugg

SEQ ID NO: 1816 - RNA, 84 nt, Tetraodon nigroviridis
cccugaugugaggguaugugcccuuggacuacaucguggaagccagcaccaugcaguccaugggcauauacacuuuccucaagg

SEQ ID NO: 1817 - RNA, 73 nt, Rattus norvegicus
gagggggaagacgggagaagagaagggagugguuuuugggugccucacuccuccccucccgucuuguucucuc

SEQ ID NO: 1818 - RNA, 84 nt, Gallus gallus
gugguggcuugguggucguauguaugacgucauuuacuuggacuuuuaggagugacaucauauguacggcugcuaaacugcugc

SEQ ID NO: 1819 - RNA, 84 nt, Xenopus tropicalis
gugguggcuuaguggucguauguaugacgucauuuacuggauuuuuuaggagugacaucauauguacggcugcuaaacugcugc

SEQ ID NO: 1820 - RNA, 104 nt, Danio rerio
acugacuugagugguggccugguggucguauguaugacgucauuuacuucaaaguuuggagugacaucauauguacggcugcuaaacugcuacauggcucauca

SEQ ID NO: 1821 - RNA, 102 nt, Fugu rubripes
cuucugauacuggugguggcuugguggucguaugcaugacgucauuuacuucgaugauuggagugacaucauauguacggcugcuaaacugcuacaagagac

SEQ ID NO: 1822 - RNA, 92 nt, Tetraodon nigroviridis
uggugguggccugguggucguaugcaugacgucauuuacuucgaugauuggagugacaucauauguacggcugcuaaaccgcuacaagagac

SEQ ID NO: 1823 - RNA, 86 nt, Mus musculus
aauugacuuagcugggaaguggggaacccuuccaugaggaguagaacacuccuuaugcaagauucccuucuaccugacugaguuga

SEQ ID NO: 1824 - RNA, 85 nt, Mus musculus
uugauacuugaaggagagguuguccguguugucuucucuuuauuuaugaugaaacauacacgggaaaccucuuuuuuaguaucaa

SEQ ID NO: 1825 - RNA, 83 nt, Rattus norvegicus
ugauacuugaaggagagguuguccguguugucuucucuuuauuuaugaugaaacauacacgggaaaccucuuuuuuaguauca

SEQ ID NO: 1826 - RNA, 84 nt, Rattus norvegicus
gcucguccucucuaauccuuugucccugggugaaaaugcuauuuguaugcaaugcacccgggcaaggauuuggggaagguaagc

SEQ ID NO: 1827 - RNA, 79 nt, Mus musculus
uccuguugggagacccuggucugcacucuaucuguaaacuuacugaagggagagcagggcaggguuucccauacagagg

SEQ ID NO: 1828 - RNA, 67 nt, Rattus norvegicus
agugggggagccaggaaguauugauguuucugccagguuagcgucaacacuugcugguuuccucucu

SEQ ID NO: 1829 - RNA, 95 nt, Homo sapiens
ggugagugcguuuccaagugugaagggacccuuccuguagugucuuauauacaauacaguaggaauguuccuucuuugccacucauacaccuuua

SEQ ID NO: 1830 - RNA, 110 nt, Homo sapiens; identical to SEQ ID NO: 1141

SEQ ID NO: 1831 - RNA, 84 nt, Homo sapiens
gucagaauaaugucaaagugcuuacagugcagguagugauaugugcaucuacugcagugaaggcacuuguagcauuauggugac

SEQ ID NO: 1832 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 1833 - RNA, 83 nt, Homo sapiens
ucaggcugugacccucuugagggaagcacuuucuguugucugaaagaagagaaagugcuuccuuuuagaggcuuacugucuga

SEQ ID NO: 1834 - RNA, 84 nt, Mus musculus
gucagaauaaugucaaagugcuuacagugcagguagugaugugugcaucuacugcagugagggcacuuguagcauuaugcugac

SEQ ID NO: 1835 - RNA, 88 nt, Mus musculus
agucaugggggcuccaaagugcuguucgugcagguaguguaauuaccugaccuacugcugagcuagcacuucccgagcccccaggaca

SEQ ID NO: 1836 - RNA, 84 nt, Rattus norvegicus
gucaggauaaugucaaagugcuuacagugcagguaguggugugugcaucuacugcagugaaggcacuuguggcauugugcugac

SEQ ID NO: 1837 - RNA, 78 nt, Rattus norvegicus
uuggccaugucaaagugcuuacagugcagguagcuucuugagaucuacugcacugcaagcacuucuuacauuaccaug

SEQ ID NO: 1838 - RNA, 87 nt, Rattus norvegicus
agucaugggggcuccaaagugcuguucgugcagguagugcauugccugaccuacugcugagcuagcacuucccgagcccccaggaca

SEQ ID NO: 1839 - RNA, 73 nt, Xenopus tropicalis
augggcuucaaagugcuguucgugcagguagcuuaauaacagaccuacugcaugggcggcacuucccaagccc

SEQ ID NO: 1840 - RNA, 77 nt, Bos taurus
cugggggcuccaaagugcuguucgugcagguagugugaucaccugaccuacugcugagccagcacuucccgagcccc

SEQ ID NO: 1841 - RNA, 138 nt, Danio rerio
ggacuuucuugaguggacuugguuggugucaauguauugucaaagugcuuacagugcagguaguauuauggaauaucuacugcaguggaggcacuucuagcaauacacuugaccauuuuaaccuuccuccaggcaucc

SEQ ID NO: 1842 - RNA, 83 nt, Danio rerio
gucacuguagugucaaagugcuuacagugcagguaguucaauauaaucuacugcaguggaggcacuucaagcuuuaccgugac

SEQ ID NO: 1843 - RNA, 74 nt, Danio rerio
guguguguuaaaagugcuguuugugcagguaguguguuuccucuacuguaggagcagcacuucacaacacacac

SEQ ID NO: 1844 - RNA, 86 nt, Fugu rubripes
gucuguguauugccaaagugcuuacagugcagguaguucuaugugacaccuacugcaauggaggcacuuacagcaguacucuugac

SEQ ID NO: 1845 - RNA, 82 nt, Fugu rubripes
gucaaaguagugucaaagugcuuacagugcagguaguuugaugacaucuacugcagugaaggcacuuucagcgcuauucuga

SEQ ID NO: 1846 - RNA, 86 nt, Tetraodon nigroviridis
gucuguguauugccaaagugcuuacagugcagguaguacuauguaacaccuacugcaauggaggcacuuacagcaguacucuugac

SEQ ID NO: 1847 - RNA, 82 nt, Tetraodon nigroviridis
gucaccguagugucaaagugcuuacagugcagguaguuaaaagaaaucuacugcagugaaggcacuuucagcacuauucuga

SEQ ID NO: 1848 - RNA, 78 nt, Homo sapiens
cuuucuacacagguugggaucgguugcaaugcuguguuucuguaugguauugcacuugucccggccuguugaguuugg

SEQ ID NO: 1849 - RNA, 75 nt, Homo sapiens
ucaucccuggguggggauuuguugcauuacuuguguucuauauaaaguauugcacuugucccggccuguggaaga

SEQ ID NO: 1850 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 1851 - RNA, 75 nt, Homo sapiens
uguugucggguggaucacgaugcaauuuugaugaguaucauaggagaaaaauugcacgguauccaucuguaaacc

SEQ ID NO: 1852 - RNA, 68 nt, Homo sapiens
ccauuacuguugcuaauaugcaacucuguugaauauaaauuggaauugcacuuuagcaauggugaugg

SEQ ID NO: 1853 - RNA, 80 nt, Mus musculus
cuuucuacacagguugggauuugucgcaaugcuguguuucucuguaugguauugcacuugucccggccuguugaguuugg

SEQ ID NO: 1854 - RNA, 91 nt, Mus musculus
ugcccauucauccacagguggggauugguggcauuacuuguguuagauauaaaguauugcacuugucccggccugaggaagaaagaggguu

SEQ ID NO: 1855 - RNA, 83 nt, Mus musculus
ggugggcgggagggacgggacguggugcaguguuguucuuuccccugccaauauugcacucgucccggccuccggcccccucg

SEQ ID NO: 1856 - RNA, 75 nt, Mus musculus
uguuaucagguggaacacgaugcaauuuugguugguguaauaggaggaaaauugcacgguauccaucuguaaacc

SEQ ID NO: 1857 - RNA, 75 nt, Mus musculus
aggccgucacuguugcuaacaugcaacucuguugaauagaaauuggaauugcacuuuagcaauggugauggaccu

SEQ ID NO: 1858 - RNA, 78 nt, Rattus norvegicus
cuuucuacacagguugggauuugucgcaaugcuguguuucuguauaguauugcacuugucccggccuguugaguuugg

SEQ ID NO: 1859 - RNA, 92 nt, Rattus norvegicus
ugcccauucauccacagguggggauuagugccauuacuuguguuagauaaaaaguauugcacuugucccggccugaggaagaaaagaggguu

SEQ ID NO: 1860 - RNA, 83 nt, Rattus norvegicus
ggugggcaggagggacgggacgcggugcaguguuguucuuuccccugccaauauugcacucgucccggccuccggcccccucg

SEQ ID NO: 1861 - RNA, 87 nt, Rattus norvegicus
uuuugcuguuaucggguggaucacgaugcaauuuugauuagaguaaugggagagaaauugcacgguauccaucuguaaaccgcagga

SEQ ID NO: 1862 - RNA, 78 nt, Ateles geoffroyi; identical to SEQ ID NO: 1848

SEQ ID NO: 1863 - RNA, 75 nt, Ateles geoffroyi; identical to SEQ ID NO: 1849

SEQ ID NO: 1864 - RNA, 78 nt, Lagothrix lagotricha; identical to SEQ ID NO: 1848

SEQ ID NO: 1865 - RNA, 75 nt, Lagothrix lagotricha; identical to SEQ ID NO: 1849

SEQ ID NO: 1866 - RNA, 78 nt, Saguinus labiatus; identical to SEQ ID NO: 1848

SEQ ID NO: 1867 - RNA, 75 nt, Saguinus labiatus; identical to SEQ ID NO: 1849

SEQ ID NO: 1868 - RNA, 78 nt, Macaca mulatta; identical to SEQ ID NO: 1848

SEQ ID NO: 1869 - RNA, 75 nt, Macaca nemestrina; identical to SEQ ID NO: 1849

SEQ ID NO: 1870 - RNA, 78 nt, Gorilla gorilla; identical to SEQ ID NO: 1848

SEQ ID NO: 1871 - RNA, 75 nt, Gorilla gorilla; identical to SEQ ID NO: 1849

SEQ ID NO: 1872 - RNA, 78 nt, Pan paniscus; identical to SEQ ID NO: 1848

SEQ ID NO: 1873 - RNA, 75 nt, Pan paniscus; identical to SEQ ID NO: 1849

SEQ ID NO: 1874 - RNA, 78 nt, Pan troglodytes; identical to SEQ ID NO: 1848

SEQ ID NO: 1875 - RNA, 74 nt, Pan troglodytes
ucaucccuggguggggauuuguugcauuacuuguguuccacaaaaguauugcacuugucccggccugugagaga

SEQ ID NO: 1876 - RNA, 78 nt, Pongo pygmaeus; identical to SEQ ID NO: 1848

SEQ ID NO: 1877 - RNA, 75 nt, Pongo pygmaeus; identical to SEQ ID NO: 1849

SEQ ID NO: 1878 - RNA, 78 nt, Lemur catta; identical to SEQ ID NO: 1848

SEQ ID NO: 1879 - RNA, 75 nt, Lemur catta
ucauccguggguggggauuuguugcauuacuuguguuauauguaaaguauugcacuugucccggccuguggaaga

SEQ ID NO: 1880 - RNA, 78 nt, Monodelphis domestica
cuuucuacacagguugggaucaguugcaaugcugugucugucuguaguauugcacuugucccggccuguugaguuugg

SEQ ID NO: 1881 - RNA, 78 nt, Gallus gallus
cuuucuacacagguugggaucaguugcaaugcugugcguuucugugguauugcacuugucccggccuguugagguugg

SEQ ID NO: 1882 - RNA, 73 nt, Gallus gallus
aggcuaauacuguugcuaacaugcaacucuguuguauaaaaauuggaauugcacuuuagcaauggugauggac

SEQ ID NO: 1883 - RNA, 78 nt, Xenopus tropicalis
cuuucuguauagguugggauugguugcaaugcuguacuauuuauguaguauugcacuugucccggccuguuuaggaug

SEQ ID NO: 1884 - RNA, 73 nt, Xenopus tropicalis
uccucuguggguggggauuuguugcacuacuguauguaugaaaaguauugcacuugucccggccuguggguga

SEQ ID NO: 1885 - RNA, 73 nt, Xenopus tropicalis
uacagaaggaucgggauguugugcacuguuguccuuucuccugccaauauugcacucgucccggccuccugcg

SEQ ID NO: 1886 - RNA, 87 nt, Xenopus tropicalis
uuuuguuguucgcggguggaucacgaugcaauuuuauuuaguuugguaggagaaaaauugcacgguauccaucuguaaaccgcagaa

SEQ ID NO: 1887 - RNA, 63 nt, Xenopus tropicalis
uuacuguugccuaugugcaaacucugugcuauauugucuagaauugcacuguagcaaugguga

SEQ ID NO: 1888 - RNA, 68 nt, Bos taurus
uggguggggauuuguugcacugcuuguguuauauauaaaguauugcacuugucccggccuguggaaga

SEQ ID NO: 1889 - RNA, 85 nt, Danio rerio
uggucccuuucugcgcagguugggauugguagcaaugcuguguguuuugaagguauugcacuugucccggccuguaaaggauugu

SEQ ID NO: 1890 - RNA, 95 nt, Danio rerio
acagcaucccuuucuuugcagguugggaucggccgcaaugcucugugcuggaaguauugcacuugucccggccugugaagagcaugggaaauugu

SEQ ID NO: 1891 - RNA, 82 nt, Danio rerio
uccuacgggcagggaggugugggauguugugcaguguuguucaaucucccgccaauauugcacucgucccggccucccugac

SEQ ID NO: 1892 - RNA, 127 nt, Danio rerio
uaaaugcaaauauuugucuugcuguuuucggguggaugacucugcaauuuuauuagugauggaaaaacuucaauaaaaauugcacgguauccaucuguaauccgcuggauuccauuaccugcguuug

SEQ ID NO: 1893 - RNA, 79 nt, Fugu rubripes
cuuuccugugcagguugggagagguagcaaugcucagagauuaugcgguauugcacuugucccggccuguggaggaagg

SEQ ID NO: 1894 - RNA, 63 nt, Fugu rubripes
cagguggggaucaguagcaaugcugugcacuggaagguauugcacuugucccggccuguguag

SEQ ID NO: 1895 - RNA, 74 nt, Tetraodon nigroviridis
cuuuccuguacagguugggagagguagcaaugcucggaguucaugugguauugcacuugucccggccuguggag

SEQ ID NO: 1896 - RNA, 72 nt, Tetraodon nigroviridis
cuucccauacagguggggaugaguagcaaugcuguguacuaggagguauugcacuugucccggccuguguag

SEQ ID NO: 1897 - RNA, 78 nt, Homo sapiens; identical to SEQ ID NO: 1315

SEQ ID NO: 1898 - RNA, 88 nt, Homo sapiens
aacugcccucaaggagcuuacaaucuagcuggggguaaaugacuugcacaugaacacaacuagacugugagcuucuagagggcaggga

SEQ ID NO: 1899 - RNA, 109 nt, Mus musculus
cuguguuugaaauggggacugcccucaaggagcuuacaaucuagcuggggguagaugacuugcacuugaacacaacuagacugugagcuucuagagggcaggggccuua

SEQ ID NO: 1900 - RNA, 88 nt, Rattus norvegicus
gacugcccucaaggagcuuacaaucuagcuggggguagaugacuugcacuugaacacaacuagacugugagcuucuagagggcagggg

SEQ ID NO: 1901 - RNA, 81 nt, Homo sapiens; identical to SEQ ID NO: 1359

SEQ ID NO: 1902 - RNA, 81 nt, Homo sapiens; identical to SEQ ID NO: 1358

SEQ ID NO: 1903 - RNA, 71 nt, Homo sapiens; identical to SEQ ID NO: 1412

SEQ ID NO: 1904 - RNA, 70 nt, Homo sapiens; identical to SEQ ID NO: 1411

SEQ ID NO: 1905 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 1906 - RNA, 88 nt, Mus musculus
uguuaaaucaggaauuguaaacaauuccuaggcaauguguauaauguugguaagucauuccuagaaauuguucacaaugccuguaaca

SEQ ID NO: 1907 - RNA, 83 nt, Rattus norvegicus
aaucaggaauuguaaacaauuccuaggcaauguguauaauguugguaagucauuccuagaaauuguucacaaugccuguaaca

SEQ ID NO: 1908 - RNA, 68 nt, Homo sapiens
ccuuugcuuuaacauggggguaccugcugugugaaacaaaaguaagugcuuccauguuucaguggagg

SEQ ID NO: 1909 - RNA, 85 nt, Homo sapiens
cucaggcugugacccuccagagggaaguacuuucuguugucugagagaaaagaaagugcuucccuuuggacuguuucgguuugag

SEQ ID NO: 1910 - RNA, 61 nt, Homo sapiens
cccucuacagggaagcgcuuucuguugucugaaagaaaagaaagugcuuccuuuuagaggg

SEQ ID NO: 1911 - RNA, 87 nt, Homo sapiens
ucucaggcugucguccucuagagggaagcacuuucuguugucugaaagaaaagaaagugcuuccuuuuagaggguuaccguuugaga

SEQ ID NO: 1912 - RNA, 87 nt, Homo sapiens
ucucaagcugugagucuacaaagggaagcccuuucuguugucuaaaagaaaagaaagugcuucucuuugguggguuacgguuugaga

SEQ ID NO: 1913 - RNA, 87 nt, Homo sapiens
ucuccugcugugacccucaagauggaagcaguuucuguugucugaaaggaaagaaagugcuuccuuuuugaggguuacuguuugaga

SEQ ID NO: 1914 - RNA, 82 nt, Mus musculus
ccuauguagcggccaucaaaguggaggcccucucuugagccugaaugagaaagugcuuccacuuugugugccacugcauggg

SEQ ID NO: 1915 - RNA, 84 nt, Mus musculus
uuccauauagccauacucaaaauggaggcccuaucuaagcuuuuaaguggaaagugcuucccuuuuguguguugccauguggag

SEQ ID NO: 1916 - RNA, 69 nt, Mus musculus
ggugagacucaaauguggggcacacuucuggacuguacauagaaagugcuacuacuuuugagucucucc

SEQ ID NO: 1917 - RNA, 82 nt, Rattus norvegicus
ccgguguaguagccaucaaaguggaggcccucucuugggcccgagcuagaaagugcuuccacuuugugugccacugcauggg

SEQ ID NO: 1918 - RNA, 68 nt, Xenopus laevis
agcacccaaaacggggcucucucuugugacugaauuaugaugagaaagugcuuucuguuuugggcguu

SEQ ID NO: 1919 - RNA, 71 nt, Xenopus laevis
cacgucggccuaacuggagcccugucucauugcagcugugaguaagugcucucuaguucgguugcugagug

SEQ ID NO: 1920 - RNA, 71 nt, Xenopus tropicalis; identical to SEQ ID NO: 1919

SEQ ID NO: 1921 - RNA, 101 nt, Danio rerio
cugcuacuguugucacuaucggugcccucacaaaggcacugacuuggaugcugcaugugguaagugcuauuuguugggguaguuucaagugaccugugcua

SEQ ID NO: 1922 - RNA, 82 nt, Danio rerio
gucacuaucgguacccucacaaaggcacugacuuggaugcuguaauugguaagugcuauuuguugggguaguuucaagugac

SEQ ID NO: 1923 - RNA, 82 nt, Danio rerio; identical to SEQ ID NO: 1922

SEQ ID NO: 1924 - RNA, 77 nt, Danio rerio
cuaucgguacccucacaaaaacacugacuuugaugcuguauuugguaagugcuauuuguugggguaguuucaaguga

SEQ ID NO: 1925 - RNA, 132 nt, Danio rerio
ggacaagguuuuaaucugcuacuauugucacuaucgguacccucacaaaggcacugacuuggaugcuguaauugguaagugcuauuuguugggguaguuucaagugaccugugcuacaagagaauguugucu

SEQ ID NO: 1926 - RNA, 132 nt, Danio rerio; identical to SEQ ID NO: 1925

SEQ ID NO: 1927 - RNA, 132 nt, Danio rerio; identical to SEQ ID NO: 1925

SEQ ID NO: 1928 - RNA, 132 nt, Danio rerio; identical to SEQ ID NO: 1925

SEQ ID NO: 1929 - RNA, 132 nt, Danio rerio; identical to SEQ ID NO: 1925

SEQ ID NO: 1930 - RNA, 132 nt, Danio rerio; identical to SEQ ID NO: 1925

SEQ ID NO: 1931 - RNA, 132 nt, Danio rerio; identical to SEQ ID NO: 1925

SEQ ID NO: 1932 - RNA, 82 nt, Danio rerio
gucucuaucgguacccucacaaaagcacugacuuugaugcuguauuugguaagugcuauuuguugggguaguuucaagugac

SEQ ID NO: 1933 - RNA, 82 nt, Danio rerio; identical to SEQ ID NO: 1932

SEQ ID NO: 1934 - RNA, 82 nt, Danio rerio; identical to SEQ ID NO: 1932

SEQ ID NO: 1935 - RNA, 82 nt, Danio rerio; identical to SEQ ID NO: 1932

SEQ ID NO: 1936 - RNA, 82 nt, Danio rerio; identical to SEQ ID NO: 1932

SEQ ID NO: 1937 - RNA, 82 nt, Danio rerio; identical to SEQ ID NO: 1932

SEQ ID NO: 1938 - RNA, 94 nt, Danio rerio
caccauugucucuaucgguacccucacaaaagcacugacuuugaugcuguauuugguaagugcuauuuguugggguaguuuuaagugacaugug

SEQ ID NO: 1939 - RNA, 94 nt, Danio rerio; identical to SEQ ID NO: 1938

SEQ ID NO: 1940 - RNA, 132 nt, Danio rerio
ggacaagguuuuaaucugcugcuauugucacuaucgguacccucacaaaggcacugacuuggaugcuguaauugguaagugcuauuuguugggguaguuucaagugaccugugcuacaagagaauguugucu

SEQ ID NO: 1941 - RNA, 132 nt, Danio rerio; identical to SEQ ID NO: 1940

SEQ ID NO: 1942 - RNA, 130 nt, Danio rerio
gacaagauuuuagucugccaccauugucucuaucgguacccucacaaaagcacugacuuugaugcuguaauugguaagugcuauuuguugggguaguuucaagugaccugugcuacaagagaauguuguc

SEQ ID NO: 1943 - RNA, 108 nt, Danio rerio
uuuaaauaaacuauuaccgucaggcuuucacaagccagccucaaaguuuguucucaaacuuuaagaaucuaguugguaagugcuauuuguugggguaguuucaaguga

SEQ ID NO: 1944 - RNA, 91 nt, Danio rerio
auugauagaaaccaguugaggucaacucuaacuuuagcaucuuucuuuuaagcaaaguagaaagugcuaucaaguugggguagauguuugc

SEQ ID NO: 1945 - RNA, 81 nt, Danio rerio
caguugaggucaacucuaacuuuagcaucuuucuuuuaagcaaaguagaaagugcuaucaaguugggguagauguuugcug

SEQ ID NO: 1946 - RNA, 81 nt, Danio rerio; identical to SEQ ID NO: 1945

SEQ ID NO: 1947 - RNA, 81 nt, Danio rerio; identical to SEQ ID NO: 1945

SEQ ID NO: 1948 - RNA, 81 nt, Danio rerio
caguugacaucaacucuaacuuuagcaucuuucuuuuaagcaaaguaaaaagugcuaucaaguugggguagauguuugcug

SEQ ID NO: 1949 - RNA, 81 nt, Danio rerio
caguugacgucaacucuaacuuuagcaucuuucuuuuaagcagaguagaaagugcuaucaaguugggguagauguuugcug

SEQ ID NO: 1950 - RNA, 81 nt, Danio rerio; identical to SEQ ID NO: 1949

SEQ ID NO: 1951 - RNA, 81 nt, Danio rerio; identical to SEQ ID NO: 1949

SEQ ID NO: 1952 - RNA, 81 nt, Danio rerio; identical to SEQ ID NO: 1949

SEQ ID NO: 1953 - RNA, 81 nt, Danio rerio
caguugacaucaacucuaacuuuagcaucuuucuuuuaagcaaaguagaaagugcuaucaaguugggguagauguuugcug

SEQ ID NO: 1954 - RNA, 81 nt, Danio rerio; identical to SEQ ID NO: 1953

SEQ ID NO: 1955 - RNA, 81 nt, Danio rerio; identical to SEQ ID NO: 1953

SEQ ID NO: 1956 - RNA, 81 nt, Danio rerio; identical to SEQ ID NO: 1953

SEQ ID NO: 1957 - RNA, 81 nt, Danio rerio; identical to SEQ ID NO: 1953

SEQ ID NO: 1958 - RNA, 81 nt, Danio rerio; identical to SEQ ID NO: 1953

SEQ ID NO: 1959 - RNA, 81 nt, Danio rerio; identical to SEQ ID NO: 1953

SEQ ID NO: 1960 - RNA, 81 nt, Danio rerio; identical to SEQ ID NO: 1953

SEQ ID NO: 1961 - RNA, 81 nt, Danio rerio; identical to SEQ ID NO: 1953

SEQ ID NO: 1962 - RNA, 81 nt, Danio rerio
caguugacaucaacucuaacuuuagcaucuuucuuuuaagcagagcagaaagugcuaucaaguugggguagauguuugcug

SEQ ID NO: 1963 - RNA, 81 nt, Danio rerio
caguugaggucaacucuaacuuuagcaucuuucuuuuaagcauaguagaaagugcuaucaaguugggguagauguuuacug

SEQ ID NO: 1964 - RNA, 81 nt, Danio rerio; identical to SEQ ID NO: 1963

SEQ ID NO: 1965 - RNA, 81 nt, Danio rerio
caguugaggucaacucuaacuuuagcaucuuucuuuuaagcaaaguagaaagugcuaucaaguugggguagauguuuguug

SEQ ID NO: 1966 - RNA, 81 nt, Danio rerio; identical to SEQ ID NO: 1965

SEQ ID NO: 1967 - RNA, 96 nt, Danio rerio
uucaaucuaaagcacauuaagaucacuucaaacaggagcauugauuuguccuuuguucauaagugcuucucuuugggguaguuuuaauaaggacag

SEQ ID NO: 1968 - RNA, 79 nt, Danio rerio
cacauuaagaucacuucaaacaggagcauugauuuguccuuuguucauaagugcuucucuuugggguaguuuuaauaag

SEQ ID NO: 1969 - RNA, 79 nt, Danio rerio; identical to SEQ ID NO: 1968

SEQ ID NO: 1970 - RNA, 79 nt, Danio rerio; identical to SEQ ID NO: 1968

SEQ ID NO: 1971 - RNA, 79 nt, Danio rerio; identical to SEQ ID NO: 1968

SEQ ID NO: 1972 - RNA, 82 nt, Danio rerio
auuaagaucacuacaaacaggagcauugauuuguccuuuguucauaagugcuucucuuugggguaguuuuaauaaggacaag

SEQ ID NO: 1973 - RNA, 73 nt, Danio rerio
auuaagaucacuucaaacaggagcauugauuuguccuuuguucauaagugcuucucuuugggguaguuuuaau

SEQ ID NO: 1974 - RNA, 82 nt, Danio rerio
auuaagaucacuucaaacaggagcauugauuuguccuuuguucauaagugcuucucuuugggguaguuuuaauaaggacaag

SEQ ID NO: 1975 - RNA, 82 nt, Danio rerio; identical to SEQ ID NO: 1974

SEQ ID NO: 1976 - RNA, 82 nt, Danio rerio; identical to SEQ ID NO: 1974

SEQ ID NO: 1977 - RNA, 82 nt, Danio rerio; identical to SEQ ID NO: 1974

SEQ ID NO: 1978 - RNA, 82 nt, Danio rerio; identical to SEQ ID NO: 1974

SEQ ID NO: 1979 - RNA, 82 nt, Danio rerio; identical to SEQ ID NO: 1974

SEQ ID NO: 1980 - RNA, 82 nt, Danio rerio; identical to SEQ ID NO: 1974

SEQ ID NO: 1981 - RNA, 82 nt, Danio rerio; identical to SEQ ID NO: 1974

SEQ ID NO: 1982 - RNA, 82 nt, Danio rerio; identical to SEQ ID NO: 1974

SEQ ID NO: 1983 - RNA, 82 nt, Danio rerio; identical to SEQ ID NO: 1974

SEQ ID NO: 1984 - RNA, 82 nt, Danio rerio; identical to SEQ ID NO: 1974

SEQ ID NO: 1985 - RNA, 82 nt, Danio rerio; identical to SEQ ID NO: 1974

SEQ ID NO: 1986 - RNA, 82 nt, Danio rerio; identical to SEQ ID NO: 1974

SEQ ID NO: 1987 - RNA, 82 nt, Danio rerio; identical to SEQ ID NO: 1974

SEQ ID NO: 1988 - RNA, 82 nt, Danio rerio
gucucuaucgguacccucacaaaagcacugacuuugaugcuguauuugguaagugcuauuuguuggcguaguuucaagugac

SEQ ID NO: 1989 - RNA, 82 nt, Danio rerio; identical to SEQ ID NO: 1988

SEQ ID NO: 1990 - RNA, 82 nt, Danio rerio; identical to SEQ ID NO: 1988

SEQ ID NO: 1991 - RNA, 81 nt, Danio rerio
caguugagaucaacucuaacuuuagcaucuuucuuuuaagcaaaguagaaagugcuaucaaauugggguagauguuugcug

SEQ ID NO: 1992 - RNA, 78 nt, Homo sapiens
gccgcaggugcucugacgagguugcacuacugugcucugagaagcagugcaaugauauugucaaagcaucugggacca

SEQ ID NO: 1993 - RNA, 115 nt, Homo sapiens
ucuguuuaucaccagauccuagaacccuaucaauauugucucugcuguguaaauaguucugaguagugcaauauugcuuauaggguuuugguguuuggaaagaacaaugggcagg

SEQ ID NO: 1994 - RNA, 88 nt, Mus musculus
ggaagacagugcaauuaaaagggggaaaaaaguaccugggauguucugagaauuucauuuuucuuguuauugccacuccugcuuggaa

SEQ ID NO: 1995 - RNA, 124 nt, Danio rerio
uugcaggucguuuaagaauuaauccuaauucuugggacccuaucaguauugccucugcuguccacuguguucagaguagugcaauauugcuaauaggguuuuagguuuuagguuguaccuucag

SEQ ID NO: 1996 - RNA, 116 nt, Danio rerio
gauaaaugcaucuugcaggcgagacccuaucaauauugccucugcuuuucucacuguuuauggaguagugcaauauugcuuauagggucuugacuuuaaggcuaaucugcaauauc

SEQ ID NO: 1997 - RNA, 73 nt, Homo sapiens
ccuggcacugagaacugaauuccauaggcugugagcucuagcaaugcccuguggacucaguucuggugcccgg

SEQ ID NO: 1998 - RNA, 95 nt, Mus musculus
ccucguugacuccgaagggcugcagcagcaauucauguuuuggaguauugccaagguucaaaacaugaagcgcugcaacaccccuucguggggaa

SEQ ID NO: 1999 - RNA, 95 nt, Rattus norvegicus
ccucgcugacuccgaagggaugcagcagcaauucauguuuuggaguauugccaagguucaaaacaugaagcgcugcaacaccccuucgugggaaa

SEQ ID NO: 2000 - RNA, 83 nt, Danio rerio
ugccugacagaagcagcacaucaauauuggcagcugcccucucucuggguugccaguaugguuugugcugcucccgucagaca

SEQ ID NO: 2001 - RNA, 78 nt, Danio rerio
gaauguacuaaagcagcacauaaauacuggaggugauugugguguuauccaguauugcuguucugcuguaguaagacc

SEQ ID NO: 2002 - RNA, 95 nt, Homo sapiens
cggccggcccuggguccaucuuccaguacaguguuggauggucuaauugugaagcuccuaacacugucugguaaagauggcucccggguggguuc

SEQ ID NO: 2003 - RNA, 72 nt, Mus musculus
ggguccaucuuccagugcaguguuggaugguugaaguaugaagcuccuaacacugucugguaaagauggccc

SEQ ID NO: 2004 - RNA, 94 nt, Rattus norvegicus
ggcugacucugaguccaucuuccagugcaguguuggaugguugaaguacgaagcuccuaacacugucugguaaagauggcccccgggucaguuc

SEQ ID NO: 2005 - RNA, 64 nt, Monodelphis domestica
uggggccaucuuccaguacagugguggauggugaagcuucuaacacugucugguaaagaugccc

SEQ ID NO: 2006 - RNA, 83 nt, Danio rerio
gucucuaggguacaucuuaccugacagugcuuggcuguucacugauguucuaacacugucugguaacgaugcacucuggugac

SEQ ID NO: 2007 - RNA, 77 nt, Homo sapiens
ccucucagggaagcuuuuugcucgaauuauguuucugauccgaauauaagacgaacaaaagguuugucugagggcag

SEQ ID NO: 2008 - RNA, 80 nt, Danio rerio
ucuacugacagagaagcuuuuuguuuguguuauguuuauuuucaaauguaagacgaacaaaaaguuuuucuguuaguagg

SEQ ID NO: 2009 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 2010 - RNA, 95 nt, Mus musculus
auccaggucuggggcaugaaccuggcauacaauguagauuucuguguuuguuaggcaacagcuacauugucugcuggguuucaggcuaccuggaa

SEQ ID NO: 2011 - RNA, 109 nt, Rattus norvegicus
ugaauauccaggucuggggcaugaaccuggcauacaauguagauuucuguguuuguuaggcaacagcuacauugucugcuggguuucaggcuaccuggaagcauuucuc

SEQ ID NO: 2012 - RNA, 110 nt, Macaca mulatta; identical to SEQ ID NO: 2009

SEQ ID NO: 2013 - RNA, 110 nt, Gorilla gorilla; identical to SEQ ID NO: 2009

SEQ ID NO: 2014 - RNA, 110 nt, Pan paniscus; identical to SEQ ID NO: 2009

SEQ ID NO: 2015 - RNA, 110 nt, Pongo pygmaeus; identical to SEQ ID NO: 2009

SEQ ID NO: 2016 - RNA, 74 nt, Monodelphis domestica
gcaugaaccuggcauacaauguagaauucuguguuuauuaaguaacagcuacauugucugcuggguuucaggcu

SEQ ID NO: 2017 - RNA, 99 nt, Gallus gallus
gacuguccagguuuggggcaugaaccuggcauacaauguagauuucuguguuuguuaagcaacagcuacauugucugcuggguuuccagcugccuggaa

SEQ ID NO: 2018 - RNA, 93 nt, Xenopus tropicalis
acccagacuuuggcgugcaccuggcauacaauguagaaaacuguguuugcaaagcaacagcuacauugucugcuggguuucaugcugaaugga

SEQ ID NO: 2019 - RNA, 110 nt, Bos taurus
ccaacauccaggucuagggcaugaaccuggcauacaauguagauuucuguguuuguugagcaacagcuacauugucugcuggguuucaggcuaccuggaaacacguucuu

SEQ ID NO: 2020 - RNA, 93 nt, Danio rerio
gucgugaaccuggcauacaauguagauuucugugugguacuaucuacagcuacauugucugcuggguuucaggccagcagaauaauucugcuc

SEQ ID NO: 2021 - RNA, 77 nt, Fugu rubripes
ccugaaccuggcauacaauguagauuucugugugcuuggauucuacagcuacauugucugcuggguuuccggauagc

SEQ ID NO: 2022 - RNA, 69 nt, Tetraodon nigroviridis
ccugaaccuggcauacaauguagauuucugugugcuuggauucuacagcuacauugucugcuggguuuc

SEQ ID NO: 2023 - RNA, 65 nt, Mus musculus
cucgaauccuuggaaccuaggugugaaugcugcuucagugcaacacaccuguucaaggauucaaa

SEQ ID NO: 2024 - RNA, 79 nt, Homo sapiens
agcaaagaaguguguugcccucuaggaaauguguguugcucugauguaauuagguugacauacguuucccugguagcca

SEQ ID NO: 2025 - RNA, 97 nt, Homo sapiens
ugaccugaaucagguaggcaguguauuguuagcuggcugcuugggucaagucagcagccacaacuacccugccacuugcuucuggauaaauucuucu

SEQ ID NO: 2026 - RNA, 63 nt, Mus musculus
cgugcagcggggcgucauccgucagcucacauagugacgcaggcagugcgaccuggcucgcac

SEQ ID NO: 2027 - RNA, 85 nt, Homo sapiens
aggccucucucuccguguucacagcggaccuugauuuaaauguccauacaauuaaggcacgcggugaaugccaagaauggggcug

SEQ ID NO: 2028 - RNA, 109 nt, Homo sapiens
aucaagauuagaggcucugcucuccguguucacagcggaccuugauuuaaugucauacaauuaaggcacgcggugaaugccaagagcggagccuacggcugcacuugaa

SEQ ID NO: 2029 - RNA, 87 nt, Homo sapiens
ugagggccccucugcguguucacagcggaccuugauuuaaugucuauacaauuaaggcacgcggugaaugccaagagaggcgccucc

SEQ ID NO: 2030 - RNA, 85 nt, Mus musculus; identical to SEQ ID NO: 2027

SEQ ID NO: 2031 - RNA, 109 nt, Mus musculus
aucaagaucagagacucugcucuccguguucacagcggaccuugauuuaaugucauacaauuaaggcacgcggugaaugccaagagcggagccuacggcugcacuugaa

SEQ ID NO: 2032 - RNA, 68 nt, Mus musculus
cucugcguguucacagcggaccuugauuuaaugucuauacaauuaaggcacgcggugaaugccaagag

SEQ ID NO: 2033 - RNA, 85 nt, Rattus norvegicus; identical to SEQ ID NO: 2027

SEQ ID NO: 2034 - RNA, 109 nt, Rattus norvegicus; identical to SEQ ID NO: 2031

SEQ ID NO: 2035 - RNA, 87 nt, Rattus norvegicus; identical to SEQ ID NO: 2029

SEQ ID NO: 2036 - RNA, 82 nt, Danio rerio
ggcucucgcuguacguguucacaguggaccuugauuuauuguauuucaauuaaggcacgcggugaaugccaacagcacagcc

SEQ ID NO: 2037 - RNA, 83 nt, Danio rerio
ccugcuuuucuucguguucacagcggaccuugauuuaaauguccauacaauuaaggcacgcggugaaugccaagagagauggc

SEQ ID NO: 2038 - RNA, 117 nt, Danio rerio
ggcucugugggauuucagacucuggcuuuccguguucacagcggaccuugauuuaaugucuuacaauuaaggcacgcggugaaugccaagagcggagccuuuuaacaucagcaggcc

SEQ ID NO: 2039 - RNA, 83 nt, Danio rerio
gguuuuugcucuuuguguucacaguggaccuugauuuaauuucaauacaauuaaggcacgcggugaaugccaagagagaagcc

SEQ ID NO: 2040 - RNA, 129 nt, Danio rerio
ggguuuugcucgugcguucuuuuugaguucucgcucugcguguucacagcggaccuugauuuaauguccauacaauuaaggcacgcggugaaugccaagagaagaaucucuccagcaacgaguuugcgc

SEQ ID NO: 2041 - RNA, 113 nt, Danio rerio
ggguggugacacaggcccgccacucugcguguucacggcggaccuugauuuaauauccauacaauuaaggcacgcggugaaugccaagagaggggucuuaaaacgacaaaccc

SEQ ID NO: 2042 - RNA, 77 nt, Fugu rubripes
gguugugucucuccguguucacagcggaccuugauuuaaugucuuacaauuaaggcacgcggugaaugccaagagau

SEQ ID NO: 2043 - RNA, 78 nt, Fugu rubripes
cuggucucuccucguguucacagcggaccuugauuuaaauguccauacaauuaaggcacgcggugaaugccaagagag

SEQ ID NO: 2044 - RNA, 83 nt, Fugu rubripes
gguuugagcucuuuguguucacaguggaccuugauuuaauuucaauacaauuaaggcacgcggugaaugccaagagagaagcc

SEQ ID NO: 2045 - RNA, 67 nt, Tetraodon nigroviridis
cucuccguguucacagcggaccuugauuuaaugucuuacaauuaaggcacgcggugaaugccaagag

SEQ ID NO: 2046 - RNA, 75 nt, Tetraodon nigroviridis
gccucuccucguguucacagcggaccuugauuuaaauguccauacaauuaaggcacgcggugaaugccaagagag

SEQ ID NO: 2047 - RNA, 80 nt, Tetraodon nigroviridis
gguuugagcucuuuguguucacaguggaccuugauuuaauuucaauacaauuaaggcacgcggugaaugccaagagagaa

SEQ ID NO: 2048 - RNA, 98 nt, Homo sapiens
agaaugggcaaaugaacaguaaauuuggaggccuggggcccucccugcugcuggagaaguguuugcacgggugggccuugucuuugaaaggaggugga

SEQ ID NO: 2049 - RNA, 85 nt, Homo sapiens
ucucaagcugugacugcaaagggaagcccuuucuguugucugaaagaagagaaagcgcuucccuuugcuggauuacgguuugaga

SEQ ID NO: 2050 - RNA, 87 nt, Homo sapiens
ucucaagcugugggucugcaaagggaagcccuuucuguugucuaaaagaagagaaagcgcuucccuuugcuggauuacgguuugaga

SEQ ID NO: 2051 - RNA, 90 nt, Homo sapiens
ucccaugcugugacccucuagaggaagcacuuucuguuuguugucugagaaaaaacaaagugcuucccuuuagaguguuaccguuuggga

SEQ ID NO: 2052 - RNA, 85 nt, Homo sapiens
cucaggcugugacacucuagagggaagcgcuuucuguugucugaaagaaaggaaagugcauccuuuuagaguguuacuguuugag

SEQ ID NO: 2053 - RNA, 87 nt, Homo sapiens
ucucaggcugugucccucuacagggaagcgcuuucuguugucugaaagaaaggaaagugcauccuuuuagaguguuacuguuugaga

SEQ ID NO: 2054 - RNA, 81 nt, Homo sapiens
caugcugugacccucuagagggaagcgcuuucuguugucugaaagaaaagaaagugcauccuuuuagagguuuacuguuug

SEQ ID NO: 2055 - RNA, 87 nt, Homo sapiens
ucucagccugugacccucuagagggaagcgcuuucuguugucugaaagaaaagaaagugcaucuuuuuagaggauuacaguuugaga

SEQ ID NO: 2056 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 2057 - RNA, 21 nt, Homo sapiens
uaguagaccguauagcguacg

SEQ ID NO: 2058 - RNA, 23 nt, Homo sapiens
aucaacagacauuaauugggcgc

SEQ ID NO: 2059 - RNA, 23 nt, Homo sapiens
aaugacacgaucacucccguuga

SEQ ID NO: 2060 - RNA, 22 nt, Homo sapiens
acccuaucaauauugucucugc

SEQ ID NO: 2061 - RNA, 21 nt, Homo sapiens
ugaaggucuacugugugccag

SEQ ID NO: 2062 - RNA, 22 nt, Homo sapiens
caugccuugaguguaggaccgu

SEQ ID NO: 2063 - RNA, 22 nt, Homo sapiens
ugugacagauugauaacugaaa

SEQ ID NO: 2064 - RNA, 22 nt, Homo sapiens
aucagcaaacauuuauugugug

SEQ ID NO: 2065 - RNA, 22 nt, Homo sapiens
caaaacuggcaauuacuuuugc

SEQ ID NO: 2066 - RNA, 22 nt, Homo sapiens
caagaaccucaguugcuuuugu

SEQ ID NO: 2067 - RNA, 22 nt, Homo sapiens
caaaaaccacaguuucuuuugc

SEQ ID NO: 2068 - RNA, 22 nt, Homo sapiens
ugucuuacucccucaggcacau

SEQ ID NO: 2069 - RNA, 21 nt, Homo sapiens
gcgacccauacuugguuucag

SEQ ID NO: 2070 - RNA, 21 nt, Homo sapiens
aacaggugacugguuagacaa

SEQ ID NO: 2071 - RNA, 21 nt, Homo sapiens
aaaacggugagauuuuguuuu

SEQ ID NO: 2072 - RNA, 19 nt, Homo sapiens
ugagcugcuguaccaaaau

SEQ ID NO: 2073 - RNA, 22 nt, Homo sapiens
caaaguuuaagauccuugaagu

SEQ ID NO: 2074 - RNA, 22 nt, Homo sapiens
ggcuggcucgcgaugucuguuu

SEQ ID NO: 2075 - RNA, 22 nt, Homo sapiens
gaaaacagcaauuaccuuugca

SEQ ID NO: 2076 - RNA, 21 nt, Homo sapiens
ugaguuggccaucugagugag

SEQ ID NO: 2077 - RNA, 23 nt, Homo sapiens
auucauuugguauaaaccgcgau

SEQ ID NO: 2078 - RNA, 22 nt, Homo sapiens
uuaugguuugccugggacugag

SEQ ID NO: 2079 - RNA, 24 nt, Homo sapiens
ucagaacaaaugccgguucccaga

SEQ ID NO: 2080 - RNA, 20 nt, Homo sapiens
agaccauggguucucauugu

SEQ ID NO: 2081 - RNA, 25 nt, Homo sapiens
aggcaccagccaggcauugcucagc

SEQ ID NO: 2082 - RNA, 21 nt, Homo sapiens
gaagugugccguggugugucu

SEQ ID NO: 2083 - RNA, 20 nt, Homo sapiens
guugugucaguuuaucaaac

SEQ ID NO: 2084 - RNA, 23 nt, Homo sapiens
acuuacagacaagagccuugcuc

SEQ ID NO: 2085 - RNA, 22 nt, Homo sapiens
uggucuaggauuguuggaggag

SEQ ID NO: 2086 - RNA, 19 nt, Homo sapiens
aggcugcggaauucaggac

SEQ ID NO: 2087 - RNA, 23 nt, Homo sapiens
uaaaucccauggugccuucuccu

SEQ ID NO: 2088 - RNA, 21 nt, Homo sapiens
aaacuacugaaaaucaaagau

SEQ ID NO: 2089 - RNA, 20 nt, Homo sapiens
aggguguuucucucaucucu

SEQ ID NO: 2090 - RNA, 21 nt, Homo sapiens
ugagcuaaaugugugcuggga

SEQ ID NO: 2091 - RNA, 23 nt, Homo sapiens
gcgaggaccccucggggucugac

SEQ ID NO: 2092 - RNA, 23 nt, Homo sapiens
gaacgccuguucuugccaggugg

SEQ ID NO: 2093 - RNA, 21 nt, Homo sapiens
uccgagccugggucucccucu

SEQ ID NO: 2094 - RNA, 22 nt, Homo sapiens
acucaaaacccuucagugacuu

SEQ ID NO: 2095 - RNA, 22 nt, Homo sapiens
agacuucccauuugaagguggc

SEQ ID NO: 2096 - RNA, 23 nt, Homo sapiens
aaacucuacuuguccuucugagu

SEQ ID NO: 2097 - RNA, 21 nt, Homo sapiens
ggcuagcaacagcgcuuaccu

SEQ ID NO: 2098 - RNA, 22 nt, Homo sapiens
uaguaccaguaccuuguguuca

SEQ ID NO: 2099 - RNA, 22 nt, Homo sapiens
agggggaaaguucuauaguccu

SEQ ID NO: 2100 - RNA, 22 nt, Homo sapiens
gugagucucuaagaaaagagga

SEQ ID NO: 2101 - RNA, 22 nt, Homo sapiens
aguauucuguaccagggaaggu

SEQ ID NO: 2102 - RNA, 21 nt, Homo sapiens
agaccuggcccagaccucagc

SEQ ID NO: 2103 - RNA, 22 nt, Homo sapiens
aaccagcaccccaacuuuggac

SEQ ID NO: 2104 - RNA, 24 nt, Homo sapiens
acugggggcuuucgggcucugcgu

SEQ ID NO: 2105 - RNA, 19 nt, Homo sapiens
aagcagcugccucugaggc

SEQ ID NO: 2106 - RNA, 21 nt, Homo sapiens
guggcugcacucacuuccuuc

SEQ ID NO: 2107 - RNA, 19 nt, Homo sapiens
aagugugcagggcacuggu

SEQ ID NO: 2108 - RNA, 21 nt, Homo sapiens
aggaggcagcgcucucaggac

SEQ ID NO: 2109 - RNA, 22 nt, Homo sapiens
uuuaggauaagcuugacuuuug

SEQ ID NO: 2110 - RNA, 21 nt, Homo sapiens
uugaaacaaucucuacugaac

SEQ ID NO: 2111 - RNA, 22 nt, Homo sapiens
uggugggccgcagaacaugugc

SEQ ID NO: 2112 - RNA, 23 nt, Homo sapiens
ggcagguucucacccucucuagg

SEQ ID NO: 2113 - RNA, 24 nt, Homo sapiens
ugccugggucucuggccugcgcgu

SEQ ID NO: 2114 - RNA, 22 nt, Homo sapiens
ugagaccucuggguucugagcu

SEQ ID NO: 2115 - RNA, 21 nt, Mus musculus
uaguagaccguauagcguacg

SEQ ID NO: 2116 - RNA, 21 nt, Rattus norvegicus
uaguagaccguauagcguacg

SEQ ID NO: 2117 - RNA, 23 nt, Mus musculus
aucaacagacauuaauugggcgc

SEQ ID NO: 2118 - RNA, 23 nt, Xenopus tropicalis
aaugacacgaucacucccguuga

SEQ ID NO: 2119 - RNA, 22 nt, Bos taurus
caugccuugaguguaggaccgu

SEQ ID NO: 2120 - RNA, 22 nt, Mus musculus
ugugacagauugauaacugaaa

SEQ ID NO: 2121 - RNA, 22 nt, Rattus norvegicus
ugugacagauugauaacugaaa

SEQ ID NO: 2122 - RNA, 21 nt, Mus musculus
gcgacccauacuugguuucag

SEQ ID NO: 2123 - RNA, 23 nt, Mus musculus
aaugacaccacauauauggcagc

SEQ ID NO: 2124 - RNA, 24 nt, Danio rerio
aaugacacguuuucucccggaucg

SEQ ID NO: 2125 - RNA, 22 nt, Homo sapiens
ugaaggucuacugugugccagg

SEQ ID NO: 2126 - RNA, 22 nt, Homo sapiens
cgucuuacccagcaguguuugg

SEQ ID NO: 2127 - RNA, 22 nt, Mus musculus
cgucuuacccagcaguguuugg

SEQ ID NO: 2128 - RNA, 21 nt, Homo sapiens
gcgacccacucuugguuucca

SEQ ID NO: 2129 - RNA, 23 nt, Mus musculus
auugugucaauaugcgaugaugu

SEQ ID NO: 2130 - RNA, 22 nt, Homo sapiens
acucaaaaugggggcgcuuucc

SEQ ID NO: 2131 - RNA, 22 nt, Mus musculus
acucaaaauggaggcccuaucu

SEQ ID NO: 2132 - RNA, 22 nt, Homo sapiens
uggugggcacagaaucuggacu

SEQ ID NO: 2133 - RNA, 96 nt, Homo sapiens
ugguacuuggagagauaguagaccguauagcguacgcuuuaucugugacguauguaacacgguccacuaacccucaguaucaaauccauccccgag

SEQ ID NO: 2134 - RNA, 85 nt, Homo sapiens
gcacauuguaggccucauuaaauguuuguugaaugaaaaaaugaaucaucaacagacauuaauugggcgccugcucugugaucuc

SEQ ID NO: 2135 - RNA, 87 nt, Homo sapiens
gaaagcgcuuuggaaugacacgaucacucccguugagugggcacccgagaagccaucgggaaugucguguccgcccagugcucuuuc

SEQ ID NO: 2136 - RNA, 115 nt, Homo sapiens; identical to SEQ ID NO: 1993

SEQ ID NO: 2137 - RNA, 89 nt, Homo sapiens; identical to SEQ ID NO: 1045

SEQ ID NO: 2138 - RNA, 91 nt, Homo sapiens
cgacuugcuuucucuccuccaugccuugaguguaggaccguuggcaucuuaauuacccucccacacccaaggcuugcaaaaaagcgagccu

SEQ ID NO: 2139 - RNA, 97 nt, Homo sapiens
cagaucucagacaucucggggaucaucaugucacgagauaccagugugcacuugugacagauugauaacugaaaggucugggagccacucaucuuca

SEQ ID NO: 2140 - RNA, 106 nt, Homo sapiens
cccagccuggcacauuaguaggccucaguaaauguuuauuagaugaauaaaugaaugacucaucagcaaacauuuauugugugccugcuaaagugagcuccacagg

SEQ ID NO: 2141 - RNA, 97 nt, Homo sapiens
ugcagggagguauuaaguuggugcaaaaguaauugugauuuuugccauuaaaaguaacgacaaaacuggcaauuacuuuugcaccaaaccugguauu

SEQ ID NO: 2142 - RNA, 97 nt, Homo sapiens
ugugauguguauuagguuugugcaaaaguaauugggguuuuuugccguuaaaaguaauggcaaaacuggcaauuacuuuugcaccaaacuaauauaa

SEQ ID NO: 2143 - RNA, 97 nt, Homo sapiens
ccuagaauguuauuaggucggugcaaaaguaauugcgaguuuuaccauuacuuucaauggcaaaacuggcaauuacuuuugcaccaacguaauacuu

SEQ ID NO: 2144 - RNA, 97 nt, Homo sapiens
cagacuauauauuuagguuggcgcaaaaguaauugugguuuuggccuuuauuuucaauggcaagaaccucaguugcuuuugugccaaccuaauacuu

SEQ ID NO: 2145 - RNA, 97 nt, Homo sapiens
aaacaaguuauauuagguuggugcaaaaguaauugugguuuuugccuguaaaaguaauggcaaaaaccacaguuucuuuugcaccagacuaauaaag

SEQ ID NO: 2146 - RNA, 97 nt, Homo sapiens
gagagggaagauuuagguuggugcaaaaguaauugugguuuuugccauugaaaguaauggcaaaaaccacaguuucuuuugcaccaaccuaauaaaa

SEQ ID NO: 2147 - RNA, 97 nt, Homo sapiens
ugaugcuuugcuggcuggugcagugccugagggaguaagagcccuguuguuguaagauagugucuuacucccucaggcacaucuccaacaagucucu

SEQ ID NO: 2148 - RNA, 97 nt, Homo sapiens
ugaugcuuugcuggcuggugcagugccugagggaguaagagcccuguuguugucagauagugucuuacucccucaggcacaucuccagcgagucucu

SEQ ID NO: 2149 - RNA, 96 nt, Homo sapiens
agaugugcucuccuggcccaugaaaucaagcgugggugagaccuggugcagaacgggaaggcgacccauacuugguuucagaggcugugagaauaa

SEQ ID NO: 2150 - RNA, 96 nt, Homo sapiens
aaccauucaaauauaccacaguuuguuuaaccuuuugccuguugguugaagaugccuuucaacaggugacugguuagacaaacugugguauauaca

SEQ ID NO: 2151 - RNA, 68 nt, Homo sapiens
cuucaauuuuauuuuaaaacggugagauuuuguuuugucugagaaaaucucgcuguuuuagacugagg

SEQ ID NO: 2152 - RNA, 94 nt, Homo sapiens
gugugugugugugugugugguuauuuugguauaguagcucuagacucuauuauaguuuccugagcugcuguaccaaaauaccacaaacgggcug

SEQ ID NO: 2153 - RNA, 97 nt, Homo sapiens
cuucauccaccaguccuccaggaacaucaaggaucuuaaacuuugccagagcuacaaaggcaaaguuuaagauccuugaaguuccugggggaaccau

SEQ ID NO: 2154 - RNA, 97 nt, Homo sapiens
ccaguggcgcaauggauaacgcgucugacuacggaucagaagauucuagguucgacuccuggcuggcucgcgaugucuguuuugccacacuugaccc

SEQ ID NO: 2155 - RNA, 97 nt, Homo sapiens
cuagauaaguuauuaggugggugcaaagguaauugcaguuuuucccauuauuuuaauugcgaaaacagcaauuaccuuugcaccaaccugauggagu

SEQ ID NO: 2156 - RNA, 96 nt, Homo sapiens
ccucaguaagaccaagcucagugugccauuuccuugucuguagccaugucuaugggcucuugaguuggccaucugagugagggccugcuuauucua

SEQ ID NO: 2157 - RNA, 98 nt, Homo sapiens
cauauuagguuaaugcaaaaguaaucgcgguuugugccagaugacgauuugaauuaauaaauucauuugguauaaaccgcgauuauuuuugcaucaac

SEQ ID NO: 2158 - RNA, 97 nt, Homo sapiens
uagggugaccagccauuaugguuugccugggacugaggaauuugcugggauaugucaguuccaggccaaccaggcugguuggucucccugaagcaac

SEQ ID NO: 2159 - RNA, 99 nt, Homo sapiens
uccagccugugcccagcagccccugagaaccacgucugcucugagcuggguacugccuguucagaacaaaugccgguucccagacgcugccagcuggcc

SEQ ID NO: 2160 - RNA, 95 nt, Homo sapiens
ucuuaucaaugagguagaccauggguucucauuguaauaguguagaauguugguuaacuguggacucccuggcucugucucaaaucuacugauuc

SEQ ID NO: 2161 - RNA, 100 nt, Homo sapiens
cccccagaaucugucaggcaccagccaggcauugcucagcccguuucccucugggggagcaaggaguggugcuggguuugucucugcugggguuucuccu

SEQ ID NO: 2162 - RNA, 96 nt, Homo sapiens
acggaagccugcacgcauuuaacaccagcacgcucaauguagucuuguaaggaacagguugaagugugccguggugugucuggaggaagcgccugu

SEQ ID NO: 2163 - RNA, 95 nt, Homo sapiens
aaagacaugcuguccacaguguguuugauaagcugacaugggacagggauucuuuucacuguugugucaguuuaucaaacccauacuuggaugac

SEQ ID NO: 2164 - RNA, 98 nt, Homo sapiens
aagucacgugcuguggcuccagcuucauaggaaggcucuugucugucaggcaguggaguuacuuacagacaagagccuugcucaggccagcccugccc

SEQ ID NO: 2165 - RNA, 79 nt, Homo sapiens
ugcaugaguucgucuuggucuaggauuguuggaggagucagaaaaacuaccccagggauccugaaguccuuugggugga

SEQ ID NO: 2166 - RNA, 94 nt, Homo sapiens
agagcaucgugcuugaccuuccacgcucucguguccacuagcaggcagguuuucugacacaggcugcggaauucaggacagugcaucauggaga

SEQ ID NO: 2167 - RNA, 83 nt, Homo sapiens
gcccuagcuugguucuaaaucccauggugccuucuccuugggaaaaacagagaaggcacuaugagauuuagaaucaaguuagg

SEQ ID NO: 2168 - RNA, 96 nt, Homo sapiens
uguauccuugguuuuuaguaguuuuacuaugaugaggugugccauccaccccaucauaguaaacuacugaaaaucaaagauacaagugccugacca

SEQ ID NO: 2169 - RNA, 95 nt, Homo sapiens
ugcucggcuguuccuaggguguuucucucaucucuggucuauaauggguuaaauaguagagaugagggcaacacccuaggaacagcagaggaacc

SEQ ID NO: 2170 - RNA, 96 nt, Homo sapiens
ucuauuugucuuaggugagcuaaaugugugcugggacacauuugagccaaaugucccagcacacauuuagcucacauaagaaaaauggacucuagu

SEQ ID NO: 2171 - RNA, 67 nt, Homo sapiens
aaaauggugagagcguugaggggaguuccagacggagaugcgaggaccccucggggucugacccaca

SEQ ID NO: 2172 - RNA, 90 nt, Homo sapiens
ucuaagaaacgcaguggucucugaagccugcaggggcaggccagcccugcacugaacgccuguucuugccagguggcagaagguugcugc

SEQ ID NO: 2173 - RNA, 96 nt, Homo sapiens
cucgggaggggcgggagggggguccccggugcucggaucucgagggugcuuauuguucgguccgagccugggucucccucuuccccccaacccccc

SEQ ID NO: 2174 - RNA, 97 nt, Homo sapiens
uuagguaauuccuccacucaaaacccuucagugacuuccaugacaugaaauaggaagucauuggaggguuugagcagaggaaugaccuguuuuaaaa

SEQ ID NO: 2175 - RNA, 97 nt, Homo sapiens
caucauaaggagccuagacuucccauuugaagguggccauuuccuaccaccuucaaaugguaaguccaggcuccuucugauucaauaaaugaggagc

SEQ ID NO: 2176 - RNA, 98 nt, Homo sapiens
cucuuguucacagccaaacucuacuuguccuucugaguguaauuacguacaugcaguagcucaggagacaagcagguuuacccuguggaugagucuga

SEQ ID NO: 2177 - RNA, 96 nt, Homo sapiens
uagauugaggaaggggcugagugguaggcggugcugcugugcucugaugaagacccauguggcuagcaacagcgcuuaccuuuugucucugggucc

SEQ ID NO: 2178 - RNA, 97 nt, Homo sapiens
aaugcuguuucaagguaguaccaguaccuuguguucaguggaaccaagguaaacacaagguauugguauuaccuugagauagcauuacaccuaagug

SEQ ID NO: 2179 - RNA, 85 nt, Homo sapiens
aggguagagggaugagggggaaaguucuauaguccuguaauuagaucucaggacuauagaacuuucccccucaucccucugcccu

SEQ ID NO: 2180 - RNA, 97 nt, Homo sapiens
uacuuauuacugguagugagucucuaagaaaagaggaggugguuguuuuccuccucuuuucuuugagacucacuaccaauaauaagaaauacuacua

SEQ ID NO: 2181 - RNA, 97 nt, Homo sapiens
aacuuaacaucaugcuaccucuuuguaucauauuuuguuauucuggucacagaaugaccuaguauucuguaccagggaagguaguucuuaacuauau

SEQ ID NO: 2182 - RNA, 75 nt, Homo sapiens
guggggagccugguuagaccuggcccagaccucagcuacacaagcugauggacugagucaggggccacacucucc

SEQ ID NO: 2183 - RNA, 97 nt, Homo sapiens
aaacccacaccacugcauuuuggccaucgaggguuggggcuuggugucaugccccaagauaaccagcaccccaacuuuggacagcauggauuagucu

SEQ ID NO: 2184 - RNA, 99 nt, Homo sapiens
uggcuaagguguuggcucgggcuccccacugcaguuacccuccccucggcguuacugagcacugggggcuuucgggcucugcgucugcacagauacuuc

SEQ ID NO: 2185 - RNA, 94 nt, Homo sapiens
gaucaggagucugccaguggagucagcacaccugcuuuucaccugugaucccaggagaggaagcagcugccucugaggccucaggcucaguggc

SEQ ID NO: 2186 - RNA, 96 nt, Homo sapiens
aggaaguguuggccuguggcugcacucacuuccuucagccccaggaagccuuggucgggggcaggagggagggucaggcagggcugggggccugac

SEQ ID NO: 2187 - RNA, 94 nt, Homo sapiens
aucacagacaccuccaagugugcagggcacuggugggggccggggcaggcccagcgaaagugcaggaccuggcacuuagucggaagugagggug

SEQ ID NO: 2188 - RNA, 96 nt, Homo sapiens
cagugcuggggucucaggaggcagcgcucucaggacgucaccaccauggccugggcucugcuccuccucacccuccucacucagggcacaggugau

SEQ ID NO: 2189 - RNA, 97 nt, Homo sapiens
aaucuaucacugcuuuuuaggauaagcuugacuuuuguucaaauaaaaaugcaaaaggaaaguguauccuaaaaggcaaugacaguuuaauguguuu

SEQ ID NO: 2190 - RNA, 96 nt, Homo sapiens
uucauuccuucaguguugaaacaaucucuacugaaccagcuucaaacaaguucacuggaguuuguuucaauauugcaagaaugauaagauggaagc

SEQ ID NO: 2191 - RNA, 81 nt, Homo sapiens
ggguaaguggaaagauggugggccgcagaacaugugcugaguucgugccauaugucugcugaccaucaccuuuagaagccc

SEQ ID NO: 2192 - RNA, 98 nt, Homo sapiens
guguaguagagcuaggaggagaggguccuggagaagcguggaccgguccggguggguuccggcagguucucacccucucuaggccccauucuccucug

SEQ ID NO: 2193 - RNA, 89 nt, Homo sapiens
ggagaggcugugcuguggggcaggcgcaggccugagcccugguuucgggcugccugggucucuggccugcgcgugacuuugggguggcu

SEQ ID NO: 2194 - RNA, 118 nt, Homo sapiens
gccuuggugcugauuccugggcucugaccugagaccucuggguucugagcugugauguugcucucgagcugggaucuccggggucuugguucagggccggggccucuggguuccaagc

SEQ ID NO: 2195 - RNA, 82 nt, Mus musculus
ugguacuuggagagauaguagaccguauagcguacgcuuuaucugugacguauguaacacgguccacuaacccucaguauca

SEQ ID NO: 2196 - RNA, 76 nt, Rattus norvegicus
cuuggagagauaguagaccguauagcguacgcuuuaucugugacguauguaacacgguccacuaacccucaguauc

SEQ ID NO: 2197 - RNA, 76 nt, Mus musculus
cauuguaggccucauuaaauguuuguugaaugaaaaaaugaaucaucaacagacauuaauugggcgccugcucugu

SEQ ID NO: 2198 - RNA, 77 nt, Xenopus tropicalis
gccuuggaaugacacgaucacucccguugagccgaaacucacagcgccaucgggaauaucgugucgcuccaaagcuc

SEQ ID NO: 2199 - RNA, 86 nt, Bos taurus
gacuugcuuucucucuuacaugccuugaguguaggaccguuggcaucuuaauuacccucccacacccaaggcuugcaggagagcca

SEQ ID NO: 2200 - RNA, 85 nt, Mus musculus
ggaucucagacgucucggggaucaucaugucacgagauaccacugugcccuugugacagauugauaacugaaaggucugggagcc

SEQ ID NO: 2201 - RNA, 79 nt, Rattus norvegicus
ucucagacgucucggggaucaucaugucacgagauaccacugcgcacuugugacagauugauaacugaaaggucuggga

SEQ ID NO: 2202 - RNA, 98 nt, Mus musculus
gugcucuuguggcccaugaaaucaagcuugggugagaccuggugcagaacaggaaggcgacccauacuugguuucaguggcugcaagaaugacugcau

SEQ ID NO: 2203 - RNA, 107 nt, Mus musculus
acugcugcaguggcagcuugguugucauaugugugaugacacuuucuaaagucuuccagaaugacaccacauauauggcagcuaaacuguuacauggaacaacaagu

SEQ ID NO: 2204 - RNA, 90 nt, Danio rerio
ugaugcugaucuggaaugacacguuuucucccggaucgccagaaauauguuucgccaccgggaacuucgugucagccaagauuggcauca

SEQ ID NO: 2205 - RNA, 68 nt, Homo sapiens
cccucgucuuacccagcaguguuugggugcgguugggagucucuaauacugccggguaaugauggagg

SEQ ID NO: 2206 - RNA, 69 nt, Mus musculus
cccucgucuuacccagcaguguuugggugcugguugggagucucuaauacugccggguaaugauggagg

SEQ ID NO: 2207 - RNA, 96 nt, Homo sapiens
ggggacugccgggugacccuggaaauccagaguggguggggccagucugaccguuucuaggcgacccacucuugguuuccaggguugcccuggaaa

SEQ ID NO: 2208 - RNA, 96 nt, Mus musculus
ugauauuaugccaugacauugugucaauaugcgaugauguguugugauggcacagcgucaucacguggugacgcaacaucaugacguaagacauca

SEQ ID NO: 2209 - RNA, 69 nt, Homo sapiens
gggauacucaaaaugggggcgcuuuccuuuuugucuguacugggaagugcuucgauuuugggguguccc

SEQ ID NO: 2210 - RNA, 84 nt, Mus musculus; identical to SEQ ID NO: 1915

SEQ ID NO: 2211 - RNA, 84 nt, Homo sapiens
acgucagggaaaggauucugcugucggucccacuccaaaguucacagaauggguggugggcacagaaucuggacucugcuugug

SEQ ID NO: 2212 - RNA, 21 nt, Xenopus tropicalis
uggaauguaaagaaguaugua

SEQ ID NO: 2213 - RNA, 23 nt, Mus musculus
uggaagacuagugauuuuguugu

SEQ ID NO: 2214 - RNA, 23 nt, Mus musculus
uacccuguagaaccgaauuugug

SEQ ID NO: 2215 - RNA, 23 nt, Rattus norvegicus
uacccuguagauccgaauuugug

SEQ ID NO: 2216 - RNA, 23 nt, Xenopus laevis
uaaggugcaucuagugcaguuag

SEQ ID NO: 2217 - RNA, 21 nt, Rattus norvegicus
caaagugcucauagugcaggu

SEQ ID NO: 2218 - RNA, 23 nt, Xenopus laevis
caaagugcucauagugcagguag

SEQ ID NO: 2219 - RNA, 22 nt, Xenopus tropicalis
uggcucaguucagcaggaacag

SEQ ID NO: 2220 - RNA, 21 nt, Xenopus tropicalis
uucaaguaauccaggauaggc

SEQ ID NO: 2221 - RNA, 21 nt, Mus musculus
uucacaguggcuaaguuccgc

SEQ ID NO: 2222 - RNA, 23 nt, Mus musculus
uagcaccauuugaaaucaguguu

SEQ ID NO: 2223 - RNA, 22 nt, Mus musculus
uguaaacauccucgacuggaag

SEQ ID NO: 2224 - RNA, 22 nt, Rattus norvegicus
uguaaacauccuacacucagcu

SEQ ID NO: 2225 - RNA, 23 nt, Mus musculus
uaagugcuuccauguuuugguga

SEQ ID NO: 2226 - RNA, 22 nt, Xenopus tropicalis
cagugcaauauuaaaagggcau

SEQ ID NO: 2227 - RNA, 22 nt, Xenopus tropicalis
uugguccccuucaaccagcugc

SEQ ID NO: 2228 - RNA, 23 nt, Monodelphis domestica
uguaguguuuccuacuuuaugga

SEQ ID NO: 2229 - RNA, 23 nt, Danio rerio
uguaguguuuccuacuuuaugga

SEQ ID NO: 2230 - RNA, 22 nt, Mus musculus
ugagaacugaauuccauaggcu

SEQ ID NO: 2231 - RNA, 21 nt, Fugu rubripes
ugguucuagacuugccaacua

SEQ ID NO: 2232 - RNA, 21 nt, Tetraodon nigroviridis
ugguucuagacuugccaacua

SEQ ID NO: 2233 - RNA, 22 nt, Mus musculus
aacuggccuacaaagucccagu

SEQ ID NO: 2234 - RNA, 22 nt, Rattus norvegicus
uagguaguuucguguuguuggg

SEQ ID NO: 2235 - RNA, 20 nt, Gallus gallus
uacaguagucugcacauugg

SEQ ID NO: 2236 - RNA, 22 nt, Mus musculus
agagguauagcgcaugggaaga

SEQ ID NO: 2237 - RNA, 22 nt, Tetraodon nigroviridis
uucccuuuguuauccuaugccu

SEQ ID NO: 2238 - RNA, 22 nt, Xenopus tropicalis
uccuucauuccaccggauccug

SEQ ID NO: 2239 - RNA, 22 nt, Mus musculus
auaagacgaacaaaagguuugu

SEQ ID NO: 2240 - RNA, 24 nt, Monodelphis domestica
agcuacaucuggcuacugggucuc

SEQ ID NO: 2241 - RNA, 22 nt, Mus musculus
uaugugggacgguaaaccgcuu

SEQ ID NO: 2242 - RNA, 22 nt, Mus musculus
ugguuuaccgucccacauacau

SEQ ID NO: 2243 - RNA, 22 nt, Rattus norvegicus
ugguuuaccgucccacauacau

SEQ ID NO: 2244 - RNA, 22 nt, Mus musculus
gcuuuaacaugggguuaccugc

SEQ ID NO: 2245 - RNA, 21 nt, Mus musculus
gccccugggccuauccuagaa

SEQ ID NO: 2246 - RNA, 22 nt, Mus musculus
uccgucucaguuacuuuauagc

SEQ ID NO: 2247 - RNA, 23 nt, Rattus norvegicus
uccgucucaguuacuuuauagcc

SEQ ID NO: 2248 - RNA, 23 nt, Mus musculus
ucucacacagaaaucgcacccgu

SEQ ID NO: 2249 - RNA, 23 nt, Rattus norvegicus
ucucacacagaaaucgcacccgu

SEQ ID NO: 2250 - RNA, 24 nt, Mus musculus
aauccuuggaaccuaggugugaau

SEQ ID NO: 2251 - RNA, 21 nt, Rattus norvegicus
aucauagaggaacauccacuu

SEQ ID NO: 2252 - RNA, 22 nt, Mus musculus
cuccugacuccagguccugugu

SEQ ID NO: 2253 - RNA, 22 nt, Rattus norvegicus
cuccugacuccagguccugugu

SEQ ID NO: 2254 - RNA, 23 nt, Mus musculus
agcucggucugaggccccucagu

SEQ ID NO: 2255 - RNA, 22 nt, Mus musculus
uggcaguguauuguuagcuggu

SEQ ID NO: 2256 - RNA, 22 nt, Mus musculus
uaugugccuuuggacuacaucg

SEQ ID NO: 2257 - RNA, 21 nt, Mus musculus
ucacuccuccccucccgucuu

SEQ ID NO: 2258 - RNA, 22 nt, Mus musculus
agaggcuggccgugaugaauuc

SEQ ID NO: 2259 - RNA, 22 nt, Rattus norvegicus
agaggcuggccgugaugaauuc

SEQ ID NO: 2260 - RNA, 21 nt, Mus musculus
cccagauaauagcacucucaa

SEQ ID NO: 2261 - RNA, 22 nt, Mus musculus
aauccuuugucccugggugaaa

SEQ ID NO: 2262 - RNA, 84 nt, Xenopus tropicalis
accuguuuggaguacauacuucuuuauguacccauaugaacauacaaugcuauggaauguaaagaaguauguauuuuuaucagg

SEQ ID NO: 2263 - RNA, 71 nt, Xenopus tropicalis
ugggagacauacuucuuuauauguccauauggaagugccaaugcuauggaauguaaagaaguauguaucuc

SEQ ID NO: 2264 - RNA, 108 nt, Mus musculus
uuggauguuggccuaguucuguguggaagacuagugauuuuguuguuuuuagauaacuaaaacgacaacaaaucacagucugccauauggcacaggccaccucuacag

SEQ ID NO: 2265 - RNA, 97 nt, Mus musculus
ggucgggccagccccguuuggaagacuagugauuuuguuguugugucucuguauccaacaacaagucccagucugccacauggugcuggucauuuca

SEQ ID NO: 2266 - RNA, 68 nt, Mus musculus
uauauacccuguagaaccgaauuugugugguacccacauagucacagauucgauucuaggggaauaua

SEQ ID NO: 2267 - RNA, 110 nt, Rattus norvegicus; identical to SEQ ID NO: 1134

SEQ ID NO: 2268 - RNA, 79 nt, Xenopus laevis
ccuuguguuaaggugcaucuagugcaguuagugacauagcguagcaucuacugcccuaaaugcuccuuuuggcacaggg

SEQ ID NO: 2269 - RNA, 72 nt, Rattus norvegicus
guagugccaaagugcucauagugcagguagguuuugcugcacucuacugcagugugagcacuucugguacuc

SEQ ID NO: 2270 - RNA, 74 nt, Xenopus laevis
gcaguuccaaagugcucauagugcagguaguuguauucauguucuacuguaauaugggcacuuacaguacugcu

SEQ ID NO: 2271 - RNA, 67 nt, Xenopus tropicalis
cucuugugccuacugaacugauaucaguucuauuucacacacuggcucaguucagcaggaacaggag

SEQ ID NO: 2272 - RNA, 85 nt, Xenopus tropicalis
ggcugcugccugguucaaguaauccaggauaggcuguuuccucaaagcacggccuacucuugauuacuuguuucaggaaguagcu

SEQ ID NO: 2273 - RNA, 80 nt, Xenopus tropicalis
ugggcgcucgcuucaaguaauccaggauaggcuguaaugucuccugucagccuuuucucgauuacuugggaccagagcca

SEQ ID NO: 2274 - RNA, 87 nt, Mus musculus
uggccugaggagcagggcuuagcugcuugugagcaagguccacagcaaagucguguucacaguggcuaaguuccgcccccuggaccc

SEQ ID NO: 2275 - RNA, 71 nt, Mus musculus
aggaagcugguuucauauggugguuuagauuuaaauagugauugucuagcaccauuugaaaucaguguucu

SEQ ID NO: 2276 - RNA, 81 nt, Mus musculus; identical to SEQ ID NO: 1358

SEQ ID NO: 2277 - RNA, 71 nt, Mus musculus
gcgacuguaaacauccucgacuggaagcugugaagccacaaaugggcuuucagucggauguuugcagcugc

SEQ ID NO: 2278 - RNA, 87 nt, Rattus norvegicus
ccgaguuucaguucauguaaacauccuacacucagcugucauacaugaguuggcugggauguggauguuuacgucagcugucuugga

SEQ ID NO: 2279 - RNA, 69 nt, Mus musculus
ccaccacuuaaacgugguuguacuugcuuuagaccuaagaaaguaagugcuuccauguuuuggugaugg

SEQ ID NO: 2280 - RNA, 71 nt, Xenopus tropicalis
auccguggcccuuuuucuguuguacuacuggaauuuguaauuagcagugcaauauuaaaagggcauuggcu

SEQ ID NO: 2281 - RNA, 86 nt, Xenopus tropicalis
aaugcuuugcuaaagcugguaaaauggaaccaaaucaacuguugaauggauuugguccccuucaaccagcugcagcugugcauuga

SEQ ID NO: 2282 - RNA, 74 nt, Monodelphis domestica
gcagucacccauaaaguagaaagcacuacuaacagcaauguaggguguaguguuuccuacuuuauggaugagug

SEQ ID NO: 2283 - RNA, 91 nt, Danio rerio
cguacagugcagucauccauaaaguagaaagcacuacuaaaccccucgccacaguguaguguuuccuacuuuauggaugaguguacuguug

SEQ ID NO: 2284 - RNA, 109 nt, Mus musculus
gacugagagaacuuuggccaccuggcucugagaacugaauuccauaggcugugagcucuagcagacgcccuagggacucaguucuggugcccggcugugcuauaccauc

SEQ ID NO: 2285 - RNA, 72 nt, Fugu rubripes
uguuuggcaaugguagaacucacacuggugagguagauggauccggugguucuagacuugccaacuacugcu

SEQ ID NO: 2286 - RNA, 67 nt, Tetraodon nigroviridis
uguuuggcaaugguagaacucacacuggugagguagauggauccggugguucuagacuugccaacua

SEQ ID NO: 2287 - RNA, 66 nt, Mus musculus
gagagcugggucuuugcgggcaagaugagagugucaguucaacuggccuacaaagucccaguccuc

SEQ ID NO: 2288 - RNA, 80 nt, Rattus norvegicus
cugagugaaguagguaguuucguguuguugggccuggcuuucugaacacaacaacaccaaaccaccugauucacugcagu

SEQ ID NO: 2289 - RNA, 94 nt, Gallus gallus
cuccacuccgucugcccaguguucagacuaccuguucaggacuacgagauuguacaguagucugcacauugguuaggcugugcugggauacacc

SEQ ID NO: 2290 - RNA, 108 nt, Gallus gallus
gaagcguccggagauccugcuccgucgccccaguguucagacuaccuguucaggacaaugcuguuguacaguagucugcacauugguuagacugggcaagggaaagca

SEQ ID NO: 2291 - RNA, 72 nt, Mus musculus
guuccuuuuuccuaugcauauacuucuuuguggaucuggucuaaagagguauagcgcaugggaagauggagc

SEQ ID NO: 2292 - RNA, 86 nt, Tetraodon nigroviridis
augugaccuauggacuucccuuuguuauccuaugccuggacucaaaaaaggggcugggagggcaaaggguugcccaguugucauac

SEQ ID NO: 2293 - RNA, 72 nt, Xenopus tropicalis
ugcugcuguccuucauuccaccggauccuguguaaauauaauccagauucccaguggcaugaagugcauuag

SEQ ID NO: 2294 - RNA, 77 nt, Mus musculus
ccucucagggaagcuuuuugcucgcguuauguuucucauccgaauauaagacgaacaaaagguuugucugagggcug

SEQ ID NO: 2295 - RNA, 68 nt, Monodelphis domestica
uguagauacugucucuuuccaucagcagcuacaucuggcuacugggucucugauggcaucuuagagcu

SEQ ID NO: 2296 - RNA, 63 nt, Mus musculus
aagaaaugguuuaccgucccacauacauuuugaguauguaugugggacgguaaaccgcuucuu

SEQ ID NO: 2297 - RNA, 63 nt, Rattus norvegicus; identical to SEQ ID NO: 2296

SEQ ID NO: 2298 - RNA, 68 nt, Mus musculus
ccucugcuuuaacaugggguuaccugcuguguuaaacaaaagcaagugcuuccauguuucaguggggg

SEQ ID NO: 2299 - RNA, 96 nt, Mus musculus
gagucugguuuuguuuggguuuguucuagguauggucccagggaucccagaucaaaccaggccccugggccuauccuagaaccaaccuaaacccgu

SEQ ID NO: 2300 - RNA, 98 nt, Mus musculus
caauuguacuuggugugauuauaaagcaaugagacugauugucauaugucguuugugggauccgucucaguuacuuuauagccauaccugguaucuua

SEQ ID NO: 2301 - RNA, 98 nt, Rattus norvegicus
cacuuguacucggugugauuauaaagcaaugagacugauugucaugugucguuugugggauccgucucaguuacuuuauagccauaccugguaucuua

SEQ ID NO: 2302 - RNA, 99 nt, Mus musculus
gaaaaugggcucaaggugaggggugcuaucugugauugagggacauggucaauggaauugucucacacagaaaucgcacccgucaccuuggccugcuga

SEQ ID NO: 2303 - RNA, 99 nt, Rattus norvegicus; identical to SEQ ID NO: 2302

SEQ ID NO: 2304 - RNA, 86 nt, Rattus norvegicus
uuugguauuuaaaagguggauauuccuucuaugguuacgugcuuccuggauaaucauagaggaacauccacuuuuucaguaucaaa

SEQ ID NO: 2305 - RNA, 66 nt, Mus musculus
agggcuccugacuccagguccuguguguuaccucgaaauagcacuggacuuggagucagaaggccu

SEQ ID NO: 2306 - RNA, 65 nt, Rattus norvegicus
gggcuccugacuccagguccuguguguuaccucgaaauagcacuggacuuggagucagaaggccu

SEQ ID NO: 2307 - RNA, 109 nt, Mus musculus
acuugugaggaaauaaaggaaguuaggcugaggggcagagagcgagacuuuucuauuuuccaaaagcucggucugaggccccucagucuugcuuccuaccccgcgcuug

SEQ ID NO: 2308 - RNA, 91 nt, Mus musculus
cugugugugauggcuuggcaguguauuguuagcugguugaguaugugagcggcaccagcuaacaugcgacugcucuccuauugcacacaca

SEQ ID NO: 2309 - RNA, 82 nt, Mus musculus
cucccuggugugagcguaugugccuuuggacuacaucgugaacgcagcaccaugcaguccacgggcauauacacuugccuca

SEQ ID NO: 2310 - RNA, 73 nt, Mus musculus; identical to SEQ ID NO: 1817

SEQ ID NO: 2311 - RNA, 73 nt, Mus musculus
acuuggagagaggcuggccgugaugaauucgauucaucuaaacgagucauacacggcucuccucucuucuagu

SEQ ID NO: 2312 - RNA, 79 nt, Rattus norvegicus
gauacuuggagagaggcuggccgugaugaauucgauucaucuaaacgagucauacacggcucuccucucuucuaguguc

SEQ ID NO: 2313 - RNA, 109 nt, Mus musculus
uucgggugagagugagaauccucucucccagauaauagcacucucaaacaaguuuccauguuguuugaaaggcuguuucuuggucagaagacucucaauuucuucugga

SEQ ID NO: 2314 - RNA, 109 nt, Mus musculus
cuucugcucugcucauccucucuaauccuuugucccugggugaaaaugcuauuuguaugcaaugcacccgggcaaggauuuggggaaggugagccugaucugcauggag

SEQ ID NO: 2315 - RNA, 23 nt, Mus musculus
aaugacacgaucacucccguuga

SEQ ID NO: 2316 - RNA, 23 nt, Rattus norvegicus
aaugacacgaucacucccguuga

SEQ ID NO: 2317 - RNA, 23 nt, Mus musculus
ugaagguccuacugugugccagg

SEQ ID NO: 2318 - RNA, 21 nt, Rattus norvegicus
ugaaggucuacugugugccag

SEQ ID NO: 2319 - RNA, 22 nt, Mus musculus
caugccuugaguguaggaccgu

SEQ ID NO: 2320 - RNA, 22 nt, Rattus norvegicus
caugccuugaguguaggacugu

SEQ ID NO: 2321 - RNA, 22 nt, Mus musculus
uccgagccugggucucccucuu

SEQ ID NO: 2322 - RNA, 85 nt, Mus musculus
aaagugcuuuggaaugacacgaucacucccguugagugggcacccaagaagccaucgggaaugucguguccgcccagugcucuuu

SEQ ID NO: 2323 - RNA, 83 nt, Rattus norvegicus
agugcuuuggaaugacacgaucacucccguugagugggcacccaagaagccaucgggaauaucguguccgcccagugcucuuu

SEQ ID NO: 2324 - RNA, 83 nt, Mus musculus
cgccagggccuuguacaugguaggcuuucauucauuuuuugcacauucggugaagguccuacugugugccaggcccugugcca

SEQ ID NO: 2325 - RNA, 84 nt, Rattus norvegicus
ggccuccagggccuuguacaugguaggcuuucauucauuguuugcacauucggugaaggucuacugugugccaggcccugugcc

SEQ ID NO: 2326 - RNA, 96 nt, Mus musculus
cagauuugcuuuuucucuuccaugccuugaguguaggaccguugacaucuuaauuacccucccacacccaaggcuugcaggagagcaagccuucuc

SEQ ID NO: 2327 - RNA, 79 nt, Rattus norvegicus
uuuucucuuccaugccuugaguguaggacuguugacaucuuaauuacccucccacacccaaggcuugcaggagagcaag

SEQ ID NO: 2328 - RNA, 92 nt, Mus musculus
ucgggaggggcgggagggggguccccggugcucggaucucgagggugcuuauuguucgguccgagccugggucucccucuuccccccauccc

(19) United States
(12) Patent Application Publication
(10) Pub. No.: US 2010/0305188 A1
(43) Pub. Date: Dec. 2, 2010

(54) NUCLEIC ACID CAPABLE OF REGULATING THE PROLIFERATION OF CELL

(75) Inventors: Haruo Nakano, Machida-shi (JP); Tatsuya Miyazawa, Machida-shi (JP); Yoji Yamada, Sunto-gun (JP); Tetsuo Yoshida, Machida-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: KYOWA HAKKO KIRIN CO., LTD., Tokyo (JP)

(21) Appl. No.: 12/681,417

(22) PCT Filed: Oct. 3, 2008
(86) PCT No.: PCT/JP2008/068109

§ 371 Date: Jul. 6, 2010

(30) Foreign Application Priority Data
Oct. 3, 2007 (JP) 2007-260393

(51) Int. Cl. A61K 31/7105; A61K 31/7088; C12Q 1/68; A61P 35/00

(52) U.S. Cl. 514/44 A; 514/44 R; 435/6

(57) ABSTRACT

Provided are an agent for suppressing or promoting cell proliferation, a diagnostic reagent or therapeutic drug for a disease resulting from a cell proliferation abnormality, an agent for inducing apoptosis, an agent for suppressing or promoting the expression of a target gene for a nucleic acid such as a microRNA, and a method of suppressing or promoting cell proliferation.

### TECHNICAL FIELD

The present invention relates to an agent for suppressing or promoting cell proliferation, a diagnostic reagent or therapeutic drug for a disease resulting from a cell proliferation abnormality, a method of suppressing or promoting cell proliferation, a method of suppressing or promoting the expression of a target gene for a nucleic acid, and a method of screening an gent for suppressing or promoting cell proliferation, based on a nucleic acid.

### BACKGROUND ART

microRNA (miRNA), which is one kind of nucleic acids, is a small non-coding single-stranded RNA of about 22 nucleotides that is not translated into a protein, and has known as being present in many types in organisms, including humans (non-patent documents 1 and 2). A miRNA is produced from a gene transcribed to a single or clustered miRNA precursor. Specifically, first, a primary-miRNA (pri-miRNA), which is a primary transcript, is transcribed from the gene, then, in stepwise processing from the pri-miRNA to a mature type micro-RNA, a precursor-miRNA (pre-miRNA) of about 70 nucleotides having a characteristic hairpin structure is produced from the pri-miRNA. Furthermore, the mature type miRNA is produced from the pre-miRNA by Dicer-mediated processing (non-patent document 3).

A mature type miRNA is thought to be involved in the post-transcriptional control of gene expression by complementarily binding to a mRNA for a target to suppress the translation of the mRNA, or to degrade the mRNA.

Although the mechanism in which miRNAs suppress the expression of target mRNAs has not been clarified in full, its outline is being elucidated through recent years' research. A miRNA suppresses the expression of the mRNA for a target by binding to a partially complementary sequence in the 3′-untranslational region (3′-UTR) of the target mRNA to suppress the translation thereof, or to degrade the target mRNA. Although the complementarity may not be complete, it has been shown that the complementarity of the 2nd to 8th nucleotides from the 5′-end of the miRNA is particularly important; this region is sometimes called the “seed sequence” of the miRNA (non-patent document 4). It has been shown that miRNAs having a same seed sequence suppress the expression of a same target mRNA even if their other sequences differ. Therefore, by making a sequence complementary to a sequence present at the 3′-end of an optionally chosen mRNA as the seed sequence, an RNA having miRNA-like activity can be designed. Usually, the term miRNA, unlike siRNA, often refers exclusively to an RNA “that occurs naturally in cells”, so a miRNA-like sequence designed as described above is sometimes particularly referred to as “an artificial miRNA”.

As of August 2007, in the miRNA database miRBase (http://microrna.sanger.ac.uk/), 533 species of miRNAs were registered for humans, and 5,071 species for all organisms. Of the miRNAs expressed in mammals, including humans, only some have their physiological functions elucidated to date, including miR-181, which is involved in hematopoietic lineage differentiation (non-patent document 5), miR-375, which is involved in insulin secretion (non-patent document 6), and the like; many have their bioactivities unclarified. However, studies using nematodes or Drosophila have shown that miRNAs play various important roles in the development and differentiation in organisms, and a report of the relation to human diseases has been reported suggesting a profound relation to cancers (non-patent document 7).

Because miRNAs are involved in the control of the expression of a wide variety of genes, abnormalities of miRNAs are supposed to be involved in various human diseases. Particularly in cancers, research has been advanced; it has been reported that in many cancers, the expression of miRNAs differs from that in normal tissues, that classification of cancers is enabled by expression profile analyses of miRNAs, and the like (non-patent document 8). It is also known that about half of the human miRNAs that have been found so far are present in chromosome aberrations or fragile portions of chromosomes known in human cancers (non-patent document 9).

Examples of relationships between cancers and miRNAs that have been reported so far include the finding that a miR-15a/miR-16 cluster is contained in the chromosome 13q14, which is deleted in B cell chronic lymphatic leukemia (B-CLL), the deletion being supposed to be a cause of B-CLL (non-patent document 10), and the finding that in B-CLL, the expression of miR-29 and miR-181 has also been decreased, one of the targets being Tcl1, which is known as a carcinogenic gene (non-patent document 11). In lung cancer, it is known that the expression of Let-7, which is a miRNA, has been decreased, one of the targets thereof being Ras, which is known as a carcinogenic gene (non-patent documents 12 and 13). It is also known that the expression of miRNAs such as miR-127 and miR-124a has been decreased due to gene modification by high methylation in cancers, and Bc16 and Cdk6, which are known as carcinogenic genes, serve as targets thereof (non-patent documents 14 and 15). Many miRNAs have their expression decreased in cancer cells; conversely, however, there are some miRNAs with gene amplification or overexpression in cancer cells. For example, in regions where gene amplification is seen in malignant lymphoma, a cluster consisting of six species of miRNAs (miR-17-92) is present; it has been known that when this miRNA cluster gene is forcedly expressed in a mouse model of human B cell lymphoma, the onset of lymphoma is promoted (non-patent document 16). It has also been shown that a gene called BIC, which has been regarded as a cancer gene candidate that does not encode a protein and is overexpressed in Hodgkin lymphoma, encodes miR-155 (non-patent document 17).

As stated above, relationships between cancers and miRNAs have recently been reported in many cases, but many of them are manifested as expression abnormalities in cancer cells, with a few reports showing functions of miRNAs, which include reports that the proliferation of cancer cell lines was inhibited by forcing cancer cell lines to express Let-7, miR-143, or miR-145 (non-patent documents 18, 19, and 20), reports that the cell cycles of cancer cell lines were halted by forcing the cancer cell lines to express miR-16 or miR-34 (non-patent documents 21 and 22), and a report that cell transformation was induced by forcing the cells to express miR-372 or miR-373 (non-patent document 23). There are only a few reports of suppressing cancer growth in animal models by administering a miRNA or a precursor thereof from outside the body to increase the expression of the miRNA, or by administering an antisense oligonucleotide thereof to decrease the expression of the miRNA; these reports include a report that tumorigenesis of a transplanted colorectal cancer cell line was suppressed by administering miR-34a (non-patent document 24) and a report that tumorigenesis of a transplanted breast cancer cell line was suppressed by administering an antisense of miR-21 (non-patent document 25).
non-patent document 1: Science, 294, 853-858, (2001)
non-patent document 2: Cell, 113, 673-676, (2003)
non-patent document 3: Nature Reviews Genetics, 5, 522-531, (2004)
non-patent document 4: Current Biology, 15, R458-R460 (2005)
non-patent document 5: Science, 303, 83-86, (2004)
non-patent document 6: Nature, 432, 226-230, (2004)
non-patent document 7: Nature Reviews Cancer, 6, 259-269, (2006)
non-patent document 8: Nature, 435, 839-843 (2005)
non-patent document 9: Proc. Natl. Acad. Sci. USA, 101, 2999-3004 (2004)
non-patent document 10: Proc. Natl. Acad. Sci. USA, 99, 15524-15529 (2002)
non-patent document 11: Cancer Research, 66, 11590-11593 (2006)
non-patent document 12: Cancer Research, 64, 3753-3756, (2004)
non-patent document 13: Cell, 120, 635-647,.(2005)
non-patent document 14: Cancer Cell, 9, 435-443, (2006)
non-patent document 15: Cancer Research, 67, 1424-1429, (2007)
non-patent document 16: Nature, 435, 823-833 (2005)
non-patent document 17: Proc. Natl. Acad. Sci. USA, 102, 3627-3632 (2005)
non-patent document 18: Cancer Research, 64, 3753-3756, (2004)
non-patent document 19: Oncology Reports, 16, 845-850, (2006)
non-patent document 20: Cancer Research, 67, 7713-7722, (2007)
non-patent document 21: Molecular and Cellular Biology, 27, 2240-2252, (2007)
non-patent document 22: Nature, 447, 1130-1134, (2007)
non-patent document 23:. Cell, 124, 1169-1181, (2006)
non-patent document 24: Proc. Natl. Acad. Sci. USA, 104, 15472-15477 (2007)
non-patent document 25: Oncogenes, 26, 2799-2803, (2007)

### DISCLOSURE OF THE INVENTION

### Problems to Be Solved by the Invention

It is expected that by identifying miRNAs expressed in various human organs, and analyzing the functions thereof to elucidate their relations to diseases, new therapeutic drugs and diagnostic reagents will be developed.

In particular, finding a miRNA that causes cancer cell proliferation or suppression is expected not only to help to understand the mechanisms of carcinogenesis, but also to lead to the development of diagnostic reagent and therapeutic drugs for human cancers, and new diagnostic methods and therapies for cancers based thereon. Furthermore, regarding diseases other than cancers, the same is expected to contribute to the development of diagnostic reagents and therapeutic drugs for diseases caused by abnormal proliferation of cells, tissue hyperplasia and the like, such as arteriosclerosis, rheumatoid arthritis, prostatic hyperplasia, blood vessel restenosis after percutaneous transvascular coronary angioplasty, fibroid lung, glomerulonephritis, and autoimmune diseases, and diagnostic methods and therapies based thereon.

It is an object of the present invention to provide nucleic acids that are useful in controlling cell proliferation and methods of utilizing the same.

### Means for Solving the Problems

The present invention relates to the following (1) to (32).
(1) An agent for suppressing or promoting cell proliferation, comprising, as an active ingredient, any one of the nucleic acids (a) to (h) below:
(a) a nucleic acid consisting of a nucleotide sequence shown by any one of SEQ ID NO:1-939, 2057-2132, 2212-2261 and 2315-2321;
(b) a nucleic acid consisting of 17 to 28 nucleotides comprising a nucleic acid consisting of a nucleotide sequence shown by any one of SEQ ID NO:1-939, 2057-2132, 2212-2261 and 2315-2321;
(c) a nucleic acid consisting of a nucleotide sequence having an identity of 90% or more to a nucleotide sequence shown by any one of SEQ ID NO:1-939, 2057-2132, 2212-2261 and 2315-2321;
(d) a nucleic acid that hybridizes under stringent conditions with a complementary strand for a nucleic acid consisting of a nucleotide sequence shown by any one of SEQ ID NO:1-939, 2057-2132, 2212-2261 and 2315-2321;
(e) a nucleic acid comprising the sequence of 2nd to 8th nucleotides of a nucleotide sequence shown by any one of SEQ ID NO:1-939, 2057-2132, 2212-2261 and 2315-2321;
(f) a nucleic acid consisting of a nucleotide sequence shown by any one of SEQ ID NO:940-2056, 2133-2211, 2262-2314 and 2322-2328;
(g) a nucleic acid consisting of a nucleotide sequence having an identity of 90% or more to a nucleotide sequence shown by any one of SEQ ID NO:940-2056, 2133-2211, 2262-2314 and 2322-2328;
(h) a nucleic acid that hybridizes under stringent conditions with a complementary strand for a nucleic acid consisting of a nucleotide sequence shown by any one of SEQ ID NO:940-2056, 2133-2211, 2262-2314 and 2322-2328.
(2) The agent according to (1), wherein the nucleic acid is a miRNA or miRNA precursor.
(3) An agent for suppressing or promoting cell proliferation, comprising, as an active ingredient, a nucleic acid consisting of a nucleotide sequence complementary to the nucleotide sequence of the nucleic acid according to (1).
(4) An agent for suppressing or promoting cell proliferation, comprising, as an active ingredient, a vector that expresses the nucleic acid according to (1) or (3).
(5) An agent for suppressing or promoting cell proliferation, comprising, as an active ingredient, a substance that suppresses the expression of a gene having a target nucleotide sequence for the nucleic acid according to (1).
(6) An agent for suppressing or promoting cell proliferation, comprising, as an active ingredient, a substance that promotes the expression of a gene having a target nucleotide sequence for the nucleic acid according to (1).
(7) The agent according to (5) or (6), wherein the substance that suppresses or promotes the expression is a nucleic acid.
(8) The agent according to (7), wherein the nucleic acid is an siRNA.
(9) An agent for suppressing or promoting cell proliferation, comprising, as an active ingredient, a vector that expresses the nucleic acid according to (7).
(10) A therapeutic drug for treating a disease resulting from a cell proliferation abnormality, comprising, as an active ingredient, the agent according to any one of (1) to (9).
(11) A therapeutic drug for treating a disease resulting from a cell proliferation abnormality, comprising, as an active ingredient, the nucleic acid according to any one of (1), (3) and (7).
(12) A reagent for diagnosing a disease resulting from a cell proliferation abnormality, comprising, as an active ingredient, a reagent that detects the expression level of the nucleic acid according to (1), a mutation of the nucleic acid, or a mutation of the genome that encodes the nucleic acid.
(13) The therapeutic drug or diagnostic reagent according to any one of (10) to (12), wherein the disease resulting from a cell proliferation abnormality is a cancer.
(14) A method of treating a disease resulting from a cell proliferation abnormality, comprising administering an effective amount of the nucleic acid according to any one of (1), (3) and (7).
(15) A method of diagnosing a disease resulting from a cell proliferation abnormality, comprising detecting the expression level of the nucleic acid according to (1), a mutation of the nucleic acid, or a mutation of the genome that encodes the nucleic acid.
(16) The method of treating or diagnosing according to (14) or (15), wherein the disease resulting from a cell proliferation abnormality is a cancer.
(17) Use of the nucleic acid according to any one of (1), (3) and (7) for the production of a therapeutic drug for a disease resulting from a cell proliferation abnormality.
(18) The use according to (17), wherein the disease resulting from a cell proliferation abnormality is a cancer.
(19) An agent for suppressing the expression of a target gene for the nucleic acid according to (1), comprising the nucleic acid as an active ingredient.
(20) An agent for promoting the expression of a target gene for the nucleic acid according to (1), comprising the nucleic acid according to (3) as an active ingredient.
(21) The agent for suppressing or promoting the expression of a target gene for the nucleic acid according to (1), comprising the vector according to (4) as an active ingredient.
(22) A method of promoting or suppressing cell proliferation, comprising using the nucleic acid according to (1) or (3).
(23) A method of promoting or suppressing cell proliferation, comprising using the vector according to (4).
(24) A method of promoting or suppressing cell proliferation, comprising using a substance that suppresses the expression of a target gene for the nucleic acid according to (1).
(25) A method of promoting or suppressing cell proliferation, comprising using a substance that promotes the expression of a target gene for the nucleic acid according to (1).
(26) The method of promoting or suppressing cell proliferation according to (24) or (25), wherein the substance that suppresses or promotes the expression is a nucleic acid.
(27) The method of promoting or suppressing cell proliferation according to (26), wherein the nucleic acid is an siRNA.
(28) A method of promoting or suppressing cell proliferation, comprising using a vector that expresses the nucleic acid according to (26).
(29) A method of suppressing the expression of a target gene for the nucleic acid according to (1), comprising using the nucleic acid.
(30) A method of promoting the expression of a target gene for the nucleic acid according to (1), comprising using the nucleic acid according to (3).
(31) A method of suppressing or promoting the expression of a target gene for the nucleic acid according to (1), comprising using the vector according to (4).
(32) A method of screening for an agent for promoting or suppressing cell proliferation, comprising using promotion or suppression of the expression or function of the nucleic acid according to (1) as an index.

### Effect of the Invention

According to the present invention, an agent for suppressing or promoting cell proliferation, a diagnostic reagent or therapeutic drug for a disease resulting from a cell proliferation abnormality, an agent for inducing apoptosis, an agent for suppressing or promoting the expression of a target gene for a nucleic acid such as a miRNA, and a method of suppressing or promoting cell proliferation can be provided.

### BRIEF DESCRIPTION OF THE DRAWINGS

FIG. 1 shows results of Western blotting for Bc1211. The miRNAs used for the individual lanes are shown below. 1; miR-negacon#2, 2; hsa-miR-337, 3; hsa-miR-491, 4; hsa-miR-342, 5; si-Bc1211.

FIG. 2 shows results of Western blotting for Birc5. The miRNAs used for the individual lanes are shown below. 1; miR-negacon#2, 2; hsa-miR-337, 3; hsa-miR-491, 4; si-Birc5.

FIG. 3 shows results of Western blotting for Bc191. The miRNAs used for the individual lanes are shown below. 1; miR-negacon#2, 2; hsa-miR-491, 3; hsa-miR-147, 4; hsa-miR-518b, 5; hsa-miR-432*, 6; hsa-miR-16, 7; si-Bc191.

FIG. 4 shows results of a tumorigenesis experiment by transplantation of DLD-1 forcedly expressing hsa-miR-147 or 342. Shown at each measuring point are the mean and standard deviation for 12 transplantation sites.

FIG. 5 shows results of a tumorigenesis experiment by transplantation of DLD-1 forcedly expressing hsa-miR-491. Shown at each measuring point are the mean and standard deviation for 12 transplantation sites.

### BEST MODE FOR CARRYING OUT THE INVENTION

The nucleic acid to be used in the present invention may be any molecule, as far as it is a molecule resulting from polymerization of a nucleotide or a molecule functionally equivalent to the nucleotide; for example, an RNA, which is a ribonucleotide polymer, a DNA, which is a deoxyribonucleotide polymer, a mixed polymer of RNA and DNA, and a nucleotide polymer, including a nucleotide analogue, can be mentioned; furthermore, the nucleic acid may be a nucleotide polymer, including a nucleic acid derivative. In addition, the nucleic acid in the present invention may be a single-stranded nucleic acid or a double-stranded nucleic acid. Double-stranded nucleic acids include double-stranded nucleic acids wherein one strand hybridizes with the other strand under stringent conditions.

In the present invention, the nucleotide analogue may be any molecule, as far as it is a molecule prepared by modifying a ribonucleotide, a deoxyribonucleotide, an RNA or a DNA in order to improve the nuclease resistance thereof, to stabilize the same, to increase the affinity thereof for a complementary chain nucleic acid, to increase the cell permeability thereof, or to visualize the same, compared with the RNA or DNA; for example, a nucleotide analogue modified at the sugar moiety thereof, a nucleotide analogue modified at phosphoric acid diester bond and the like can be mentioned.

The nucleotide analogue modified at the sugar moiety thereof may be any one, as far as an optionally chosen chemical structural substance has been added to, or substituted for, a portion or all of the chemical structure of the sugar of the nucleotide; for example, a nucleotide analogue substituted by 2′-O-methylribose, a nucleotide analogue substituted by 2′-O-propylribose, a nucleotide analogue substituted by 2′-methoxyethoxyribose, a nucleotide analogue substituted by 2′-O-methoxyethylribose, a nucleotide analogue substituted by 2′-O-[2-(guanidium)ethyl]ribose, a nucleotide analogue substituted by 2′-O-fluororibose, a bridged nucleic acid (BNA) having two cyclic structures as a result of introduction of a bridging structure into the sugar moiety, more specifically a locked nucleic acid (LNA) wherein the oxygen atom at the 2′ position and the carbon atom at the 4′ position have been bridged via methylene, and an ethylene bridged nucleic acid) (ENA) [Nucleic Acid Research, 32, e175 (2004)] can be mentioned, and a peptide nucleic acid (PNA) [Acc. Chem. Res., 32, 624 (1999)], an oxypeptide nucleic acid (OPNA) [J. Am. Chem. Soc., 123, 4653 (2001)], and a peptide ribonucleic acid (PRNA) [J. Am. Chem. Soc., 122, 6900 (2000)] and the like can also be mentioned.

The nucleotide analogue modified at phosphoric acid diester bond may be any one, as far as an optionally chosen chemical substance has been added to, or substituted for, a portion or all of the chemical structure of the phosphoric acid diester bond of the nucleotide; for example, a nucleotide analogue substituted by a phosphorothioate bond, a nucleotide analogue substituted by an N3′-P5′ phosphoamidate bond, and the like can be mentioned [SAIBO KOGAKU, 16, 1463-1473 (1997)] [RNAi Method and Antisense Method, Kodansha (2005)].

The nucleic acid derivative may be any molecule, as far as it is a molecule prepared by adding another chemical substance to the nucleic acid in order to improve the nuclease resistance thereof, to stabilize the same, to increase the affinity thereof for a complementary chain nucleic acid, to increase the cell permeability thereof, or to visualize the same, compared with the nucleic acid; for example, a 5′-polyamine conjugated derivative, a cholesterol conjugated derivative, a steroid conjugated derivative, a bile acid conjugated derivative, a vitamin conjugated derivative, a Cy5 conjugated derivative, a Cy3 conjugated derivative, a 6-FAM conjugated derivative, a biotin conjugated derivative and the like can be mentioned.

As examples of nucleic acids, the following nucleic acids (a) to (k) can be mentioned.
(a) A nucleic acid consisting of a nucleotide sequence shown by any one of SEQ ID NO:1-939, 2057-2132, 2212-2261 and 2315-2321
(b) A nucleic acid of 17 to 28 nucleotides comprising a nucleic acid consisting of a nucleotide sequence shown by any one of SEQ ID NO:1-939, 2057-2132, 2212-2261 and 2315-2321
(c) A nucleic acid consisting of a nucleotide sequence having an identity of 90% or more to a nucleotide sequence shown by any one of SEQ ID NO:1-939, 2057-2132, 2212-2261 and 2315-2321
(d) A nucleic acid that hybridizes under stringent conditions with a complementary strand for a nucleic acid consisting of a nucleotide sequence shown by any one of SEQ ID NO:1-939, 2057-2132,2212-2261 and 2315-2321
(e) A nucleic acid comprising the sequence of 2nd to 8th nucleotides of a nucleotide sequence shown by any one of SEQ ID NO:1-939, 2057-2132, 2212-2261 and 2315-2321
(f) A double-stranded nucleic acid consisting of one of the nucleic acids (a) to (e) and a nucleic acid comprising a nucleotide sequence complementary to the nucleotide sequence of the nucleic acid, or a nucleic acid comprising the double-stranded nucleic acid
(g) A double-stranded nucleic acid consisting of one of the nucleic acids (a) to (e) and a nucleic acid that hybridizes under stringent conditions with the nucleic acid, or a nucleic acid comprising the double-stranded nucleic acid
(h) A single-stranded nucleic acid having a hairpin structure wherein the double-stranded nucleic acid (f) or (g) is joined via a spacer oligonucleotide consisting of 8 to 28 nucleotides, or a nucleic acid comprising the single-stranded nucleic acid
(i) A nucleic acid consisting of a nucleotide sequence shown by any one of SEQ ID NO:940-2056, 2133-2211, 2262-2314 and 2322-2328
(j) A nucleic acid consisting of a nucleotide sequence having an identity of 90% or more to a nucleotide sequence shown by any one of SEQ ID NO:940-2056, 2133-2211, 2262-2314 and 2322-2328
(k) A nucleic acid that hybridizes under stringent conditions 15 with a complementary strand for a nucleic acid consisting of a nucleotide sequence shown by any one of SEQ ID NO:940-2056, 2133-2211, 2262-2314 and 2322-2328.

As the above-described nucleic acid, a miRNA is preferably used. A miRNA refers to a single-stranded RNA 17 to 28 nucleotides long. The peripheral genomic sequence, including a miRNA sequence has a sequence capable of forming a hairpin structure, and the miRNA can be cut out from either one strand of the hairpin. A miRNA complementarily binds to a mRNA serving as a target therefor to suppress the translation of the mRNA or promote the degradation of the mRNA, thereby mediating the post-translational control of gene expression.

As examples of miRNAs, human miRNAs consisting of a nucleotide sequence shown by any one of SEQ ID NO:1-129, 803, 819, 916, 917, 925 and 2057-2114 can be mentioned. Furthermore, as miRNAs having the same function as the function of human miRNAs consisting of a nucleotide sequence shown by any one of SEQ ID NO:1-129, 803, 819, 916, 917, 925 and 2057-2114, nucleic acids consisting of a nucleotide sequence shown by any one of SEQ ID NO:130-802, 822, 823, 826, 827, 829-840, 842, 843, 846-848, 850, 851, 2115-2122, 2212-2261 and 2315-2321, which are orthologues of the human miRNAs, can be mentioned. As specific examples of orthologues of the human miRNA of SEQ ID NO:1, those consisting of a nucleotide sequence shown by any one of SEQ ID NO:130-139 and 2212 can be mentioned. A table of correspondence of miRNAs consisting of a nucleotide sequence shown by any one of SEQ ID NO:1-129, 803, 819, 916, 917, 925 and 2057-2114 and orthologues thereof is shown in Table 1 (from Table 1-1 to Table 1-4). The biological species shown in the uppermost fields of Table 1 are as follows: has, Homo sapiens, human; mmu, Mus musculus, mouse; rno, Rattus norvegicus, rat; xla, Xenopus laevis, African clawed toad; xtr, Xenopus tropicalis, tropical clawed toad; gga, Gallus gallus, chicken; cfa, Canis familiaris, dog; mdo, Monodelphis domestica, gray short-tailed opossum; age, Ateles geoffroyi, black-handed spider monkey; ila, Lagothrix lagotricha, Humboldt's wooly monkey; sla, Saguinus labiatus, red-bellied tamarin; mml, Macaca mulatta, rhesus monkey; mne, Macaca nemestrina, pig-tailed macaque; ggo, Gorilla gorilla, gorilla; ppa, Pan paniscus, bonobo; ptr, Pan troglodytes, chimpanzee; ppy, Pongo pygmaeus, orangutan; lca, Lemur catta, ring-tailed lemur; cgr, Cricetulus griseus, Chinese hamster; bta, Bos taurus, cattle; oar, Ovis aries, sheep; ssc, Sus scrofa, swine; dre, Danio rerio, zebrafish; fru, Fugu rubripes, tiger puffer; tni, Tetraodon nigroviridis, green spotted puffer. Because human-derived miRNAs and orthologues thereof have high sequence identity, they are considered to possess similar functions. Also because miRNAs in the same subgroup as classified by alphabetical figures added to the ends of their names also share high sequence identity, they are considered to possess similar functions.

| | | | | | | | | | | | | | |
|---|---|---|---|---|---|---|---|---|---|---|---|---|---|
| SEQ ID NO: | Name of miRNA | mmu | rno | cgr | age | lla | sla | mml | mne | ggo | ppa | ptr | ppy |

Table 1-1

| | | | | | | | | | | | | |
|---|---|---|---|---|---|---|---|---|---|---|---|---|
| 1 | hsa-miR-1 | 130 | 131 | | | | | | | 132 | | |
| 2 | hsa-miR-206 | 140 | 141 | | | | | 142 | | | | 143 |
| 3 | hsa-miR-7 | 2213 | 149 | | 151 | 152 | | 153 | 154 | 155 | 156 | 157 |
| | | 148 | 150 | | | | | | | | | |
| 4 | hsa-miR-10a | 168 | 2215 | 169 | | 170 | | 171 | 172 | 174 | | |
| | | 2214 | | | | | | | 173 | 175 | | |
| 5 | hsa-miR-18a | 193 | 194 | 195 | 196 | 197 | 198 | 199 | 200 | 201 | 202 | 203 |
| | | 214 | | | | | | | | | | |
| 6 | hsa-miR-18b | | | | | | | | | | | |
| 7 | hsa-miR-18a* | 219 | | | | | | | | | | |
| 8 | hsa-miR-20b | 220 | 2217 | 221 | 222 | 223 | 224 | 225 | 226 | 227 | 228 | 229 |
| 9 | hsa-miR-106a | 239 | 253 | 240 | 255 | 241 | 242 | 243 | 244 | 245 | 246 | 247 |
| | | 252 | | 254 | | 256 | 257 | 258 | 259 | 260 | 261 | 262 |
| 10 | hsa-miR-106b | | | | | | | | | | | |
| 11 | hsa-miR-24 | 263 | 264 | | | | 265 | 266 | 267 | 268 | 269 | 270 |
| 12 | hsa-miR-25 | 279 | 280 | | 281 | | 282 | 283 | 284 | 285 | 286 | 287 |
| 13 | hsa-miR-32 | 294 | 295 | | | 296 | 297 | 298 | 299 | 300 | 301 | 302 |
| 14 | hsa-miR-26a | 306 | 307 | | 308 | | 309 | 310 | 311 | 312 | 313 | 314 |
| | | 321 | 322 | | | | | | | | | |
| 15 | hsa-miR-26b | | | | | | | | | | | |
| 16 | hsa-miR-27b | 2221 | 326 | 328 | | 329 | 330 | 331 | 332 | 333 | 334 | 335 |
| | | 325 | 327 | | | | | | | | | |
| 17 | hsa-miR-28 | 357 | 358 | 359 | 360 | 361 | 362 | 363 | 364 | 365 | 366 | 367 |
| 18 | hsa-miR-29a | 369 | 370 | 372 | 373 | 374 | | 375 | 376 | 377 | 378 | 379 |
| | | 2222 | 371 | | | | | | | | | |
| | | 395 | 396 | | | | | | | | | |
| 19 | hsa-miR-29c | | | | | | | | | | | |
| 20 | hsa-miR-30b | 2223 | 404 | 407 | 408 | | 409 | 411 | 413 | 416 | 419 | 422 |
| | | 401 | 2224 | | 446 | | 410 | 447 | 414 | 417 | 420 | |
| | | 444 | 445 | | | | | 412 | 415 | 418 | 448 | |
| | | 402 | 405 | | | | | | | | 421 | |
| | | 403 | 406 | | | | | | | | | |
| 21 | hsa-miR-30c | | | | | | | | | | | |
| 22 | hsa-miR-93 | | | 456 | 457 | 458 | 459 | 460 | 461 | 462 | 463 | 464 |
| 23 | hsa-miR-302d | 2225 | | | | | | | | | | |
| | | 466 | | | | | | | | | | |
| | | 467 | | | | | | | | | | |
| 24 | hsa-miR-95 | | | | 476 | 477 | | | 478 | 479 | 480 | 481 |
| 25 | hsa-miR-98 | 483 | 484 | 485 | | | 486 | | 487 | 488 | 489 | 490 |
| 26 | hsa-miR-130a | 493 | 494 | | | | 495 | 496 | 497 | 498 | | |
| | | 506 | 507 | | | | | | | | | |
| 27 | hsa-miR-130b | | | | | | | | | | | |
| 28 | hsa-miR-301 | 511 | 513 | | | | | | | | | |
| | | 512 | 514 | | | | | | | | | |
| 29 | hsa-miR-132 | 522 | 523 | | | | | | | | | |
| 30 | hsa-miR-212 | 530 | 531 | | | | | | | | | |
| 31 | hsa-miR-133a | | | 537 | 538 | 539 | | 540 | 541 | 542 | 543 | |
| 32 | hsa-miR-133b | | | | | | | | | | | |
| 33 | hsa-miR-134 | 552 | 553 | | | | | 554 | 555 | 556 | | 557 |
| 34 | hsa-miR-137 | | | | | | | | | | | |
| 35 | hsa-miR-142-3p | 561 | 562 | | | | | | | | | |
| 36 | hsa-miR-146a | 565 | 566 | | | | | | | | | |
| | | 2230 | | | | | | | | | | |
| 37 | hsa-miR-147 | | | | | 572 | | 573 | | 574 | 575 | 576 |
| 38 | hsa-miR-149 | 577 | | | | | | | | | | |

Table 1-2

| | | | | | | | | | | | | |
|---|---|---|---|---|---|---|---|---|---|---|---|---|
| 39 | hsa-miR-181c | 578 | 579 | | | | 580 | | 581 | 582 | 583 | |
| | | 588 | 589 | | | | | | | | | |
| 40 | hsa-miR-181d | | | | | | | | | | | |
| 41 | hsa-miR-182* | | | | | | | | | | | |
| 42 | hsa-miR-183 | 592 | 593 | | | 594 | 595 | 596 | 597 | 598 | 599 | |
| 43 | hsa-miR-184 | 607 | 608 | | | | | 609 | | | 610 | 611 |
| 44 | hsa-miR-192 | 619 | 620 | | | | | | | | | |
| 45 | hsa-miR-215 | 626 | 627 | | | | 628 | 629 | 630 | | 631 | 632 |
| 46 | hsa-miR-193a | 2233 | 636 | | | | | | | | | |
| | | 644 | | | | | | | | | | |
| 47 | hsa-miR-193b | | | | | | | | | | | |
| 48 | hsa-miR-195 | 646 | 647 | | | | | | 648 | 649 | | |
| 49 | hsa-miR-424 | | | | | | | | | | | |
| 50 | hsa-miR-497 | 651 | 652 | | | | | | | | | |
| 51 | hsa-miR-196a | 654 | 655 | 656 | 657 | | 658 | | 659 | 660 | 661 | 662 |
| | | 669 | 670 | | | | | | | | | |
| | | | 2234 | | | | | | | | | |
| 52 | hsa-miR-196b | | | | | | | | | | | |
| 53 | hsa-miR-197 | 676 | | 677 | | | | 678 | | 679 | 680 | 681 |
| 54 | hsa-miR-199a* | | | | | | | | | | | |
| 55 | hsa-miR-200a | 684 | 685 | | | | | | | | | |
| 56 | hsa-miR-202 | 2236 | | | | | | | | | | |
| 57 | hsa-miR-204 | 693 | 694 | | | 695 | | 696 | 697 | 698 | 699 | 700 |
| 58 | hsa-miR-211 | 709 | 710 | | | | 711 | 712 | | | | 713 |
| 59 | hsa-miR-205 | 715 | 716 | 717 | 718 | | | 719 | 720 | 721 | 722 | |
| 60 | hsa-miR-208 | 731 | 732 | | | | | | | | | |
| | | 2239 | | | | | | | | | | |
| 61 | hsa-miR-222 | 735 | 736 | 737 | | | | | | | | |
| 62 | hsa-miR-299-3p | 2241 | | | | | | | | | | |
| 63 | hsa-miR-299-5p | 2242 | 2243 | | | | | | | | | |
| 64 | hsa-miR-302a* | | | | | | | | | | | |
| 65 | hsa-miR-302b* | 744 | | | | | | | | | | |
| | | 2244 | | | | | | | | | | |
| 66 | hsa-miR-324-3p | 746 | 747 | | | | | | | | | |
| 67 | hsa-miR-324-5p | 748 | 749 | | | | | | | | | |
| 68 | hsa-miR-329 | 750 | 751 | | | | | | | | | |
| 69 | hsa-miR-331 | 2245 | 752 | | | | | | | | | |
| 70 | hsa-miR-337 | | | | | | | | | | | |
| 71 | hsa-miR-340 | 2246 | 2247 | | | | | | | | | |
| 72 | hsa-miR-342 | 2248 | 2249 | | | | | | | | | |
| 73 | hsa-miR-345 | 755 | 756 | | | | | | | | | |
| 74 | hsa-miR-362 | 2250 | | | | | | | | | | |
| 75 | hsa-miR-368 | | | | | | | | | | | |
| 76 | hsa-miR-371 | | | | | | | | | | | |
| 77 | hsa-miR-376a | 757 | 2251 | | | | | | | | | |
| 78 | hsa-miR-376b | | | | | | | | | | | |
| 79 | hsa-miR-377 | 758 | 759 | | | | | | | | | |
| 80 | hsa-miR-378 | 2252 | 2253 | | | | | | | | | |
| 81 | hsa-miR-380-5p | | | | | | | | | | | |
| 82 | hsa-miR-382 | 761 | 762 | | | | | | | | | |
| 83 | hsa-miR-412 | | 763 | | | | | | | | | |
| 84 | hsa-miR-422a | | | | | | | | | | | |
| 85 | hsa-miR-422b | | | | | | | | | | | |
| 86 | hsa-miR-423 | 2254 | 764 | | | | | | | | | |
| 87 | hsa-miR-431 | 765 | 766 | | | | | | | | | |
| 88 | hsa-miR-432* | | | | | | | | | | | |
| 89 | hsa-miR-433 | 768 | 769 | | | | | | | | | |
| 90 | hsa-miR-449 | 2255 | 772 | | | | | | | | | |
| | | 770 | | | | | | | | | | |
| | | 771 | | | | | | | | | | |
| 91 | hsa-miR-451 | 777 | 778 | | | | | | | | | |
| 92 | hsa-miR-452 | 783 | | | | | | | | | | |
| 93 | hsa-miR-452* | | | | | | | | | | | |
| 94 | hsa-miR-455 | 2256 | 784 | | | | | | | | | |
| 95 | hsa-miR-483 | 2257 | 791 | | | | | | | | | |
| 96 | hsa-miR-485-5p | 2258 | 2259 | | | | | | | | | |
| 97 | hsa-miR-488 | 2260 | | | | | | | | | | |
| 98 | hsa-miR-489 | | | | | | | | | | | |

Table 1-3

| | | | | | | | | | | | | |
|---|---|---|---|---|---|---|---|---|---|---|---|---|
| 99 | hsa-miR-491 | 797 | | | | | | | | | | |
| 100 | hsa-miR-493-5p | | | | | | | | | | | |
| 101 | hsa-miR-494 | 798 | 799 | | | | | | | | | |
| 102 | hsa-miR-501 | 2261 | 800 | | | | | | | | | |
| 103 | hsa-miR-504 | 801 | | | | | | | | | | |
| 104 | hsa-miR-505 | | 802 | | | | | | | | | |
| 105 | hsa-miR-506 | | | | | | | | | | | |
| 106 | hsa-miR-507 | | | | | | | | | | | |
| 107 | hsa-miR-509 | | | | | | | | | | | |
| 108 | hsa-miR-510 | | | | | | | | | | | |
| 109 | hsa-miR-511 | | | | | | | | | | | |
| 110 | hsa-miR-512-3p | | | | | | | | | | | |
| 111 | hsa-miR-512-5p | | | | | | | | | | | |
| 112 | hsa-miR-513 | | | | | | | | | | | |
| 113 | hsa-miR-516-5p | | | | | | | | | | | |
| 114 | hsa-miR-517* | | | | | | | | | | | |
| 115 | hsa-miR-517a | | | | | | | | | | | |
| 116 | hsa-miR-517c | | | | | | | | | | | |
| 117 | hsa-miR-517b | | | | | | | | | | | |
| 118 | hsa-miR-518b | | | | | | | | | | | |
| 119 | hsa-miR-518c | | | | | | | | | | | |
| 120 | hsa-miR-518d | | | | | | | | | | | |
| 121 | hsa-miR-518c* | | | | | | | | | | | |
| 122 | hsa-miR-518e | | | | | | | | | | | |
| 123 | hsa-miR-518f | | | | | | | | | | | |
| 124 | hsa-miR-520h | | | | | | | | | | | |
| 125 | hsa-miR-523 | | | | | | | | | | | |
| 126 | hsa-miR-524* | | | | | | | | | | | |
| 127 | hsa-miR-525 | | | | | | | | | | | |
| 128 | hsa-miR-526a | | | | | | | | | | | |
| 129 | hsa-miR-527 | | | | | | | | | | | |
| 819 | hsa-miR-92b | 822 | 826 | 829 | 830 | 831 | 832 | 833 | 834 | 835 | 836 | 837 |
| | | 823 | 827 | | | | | | | | | |
| 917 | hsa-miR-449b | 770 | 772 | | | | | | | | | |
| | | 771 | | | | | | | | | | |
| | | 2255 | | | | | | | | | | |
| 925 | hsa-miR-557 | | | | | | | | | | | |
| 916 | hsa-miR-563 | | | | | | | | | | | |
| 803 | hsa-miR-613 | | | | | | | | | | | |
| 2057 | hsa-miR-411 | 2115 | 2116 | | | | | | | | | |
| 2058 | hsa-miR-421 | 2117 | | | | | | | | | | |
| 2059 | hsa-miR-425-5p | 2315 | 2316 | | | | | | | | | |
| 2060 | hsa-miR-454-5p | | | | | | | | | | | |
| 2061 | hsa-miR-493-3p | 2317 | 2318 | | | | | | | | | |
| 2062 | hsa-miR-532 | 2319 | 2320 | | | | | | | | | |
| 2063 | hsa-miR-542-3p | 2120 | 2121 | | | | | | | | | |
| 2064 | hsa-miR-545 | | | | | | | | | | | |
| 2065 | hsa-miR-548a | | | | | | | | | | | |
| 2066 | hsa-miR-548b | | | | | | | | | | | |
| 2067 | hsa-miR-548d | | | | | | | | | | | |
| 2068 | hsa-miR-550 | | | | | | | | | | | |
| 2069 | hsa-miR-551b | 2122 | | | | | | | | | | |
| 2070 | hsa-miR-552 | | | | | | | | | | | |
| 2071 | hsa-miR-553 | | | | | | | | | | | |
| 2072 | hsa-miR-558 | | | | | | | | | | | |
| 2073 | hsa-miR-561 | | | | | | | | | | | |
| 2074 | hsa-miR-565 | | | | | | | | | | | |
| 2075 | hsa-miR-570 | | | | | | | | | | | |
| 2076 | hsa-miR-571 | | | | | | | | | | | |
| 2077 | hsa-miR-579 | | | | | | | | | | | |
| 2078 | hsa-miR-584 | | | | | | | | | | | |
| 2079 | hsa-miR-589 | | | | | | | | | | | |
| 2080 | hsa-miR-591 | | | | | | | | | | | |
| 2081 | hsa-miR-593 | | | | | | | | | | | |
| 2082 | hsa-miR-595 | | | | | | | | | | | |
| 2083 | hsa-miR-599 | | | | | | | | | | | |
| 2084 | hsa-miR-600 | | | | | | | | | | | |
| 2085 | hsa-miR-601 | | | | | | | | | | | |
| 2086 | hsa-miR-604 | | | | | | | | | | | |
| 2087 | hsa-miR-605 | | | | | | | | | | | |

Table 1-4

| | | |
|---|---|---|
| 2088 | hsa-miR-606 | |
| 2089 | hsa-miR-609 | |
| 2090 | hsa-miR-610 | |
| 2091 | hsa-miR-611 | |
| 2092 | hsa-miR-614 | |
| 2093 | hsa-miR-615 | 2321 |
| 2094 | hsa-miR-616 | |
| 2095 | hsa-miR-617 | |
| 2096 | hsa-miR-618 | |
| 2097 | hsa-miR-621 | |
| 2098 | hsa-miR-624 | |
| 2099 | hsa-miR-625 | |
| 2100 | hsa-miR-627 | |
| 2101 | hsa-miR-630 | |
| 2102 | hsa-miR-631 | |
| 2103 | hsa-miR-634 | |
| 2104 | hsa-miR-637 | |
| 2105 | hsa-miR-646 | |
| 2106 | hsa-miR-647 | |
| 2107 | hsa-miR-648 | |
| 2108 | hsa-miR-650 | |
| 2109 | hsa-miR-651 | |
| 2110 | hsa-miR-653 | |
| 2111 | hsa-miR-654 | |
| 2112 | hsa-miR-657 | |
| 2113 | hsa-miR-661 | |
| 2114 | hsa-miR-769-5p | |

| | | | | | | | | | | | | |
|---|---|---|---|---|---|---|---|---|---|---|---|---|
| SEQ ID NO: | lca | cfa | mdo | gga | xla | xtr | bta | oar | ssc | dre | fru | tni |

Table 1-1

| | | | | | | | | | | |
|---|---|---|---|---|---|---|---|---|---|---|
| 1 | | 133 | 134 | | 2212 | | | 137 | 138 | 139 |
| | | | 135 | | 136 | | | | | |
| 2 | | 144 | 145 | | 146 | | | 147 | | |
| 3 | | 158 | 159 | | 161 | 162 | 163 | 164 | 166 | 167 |
| | | | 160 | | | | | 165 | | |
| 4 | | 176 | 177 | | 178 | 181 | | 183 | 187 | 190 |
| | | | | | 179 | 182 | | 184 | 188 | 191 |
| | | | | | 180 | | | 185 | 189 | 182 |
| | | | | | | | | 186 | | |
| 5 | 204 | 205 | 206 | 2216 | 207 | 208 | 209 | 210 | 212 | 213 |
| | | | 215 | | 216 | 217 | | 218 | | |
| | | | | | | | | 211 | | |
| 6 | | | | | | | | | | |
| 7 | | | | | | | | | | |
| 8 | 230 | 231 | 232 | 2218 | 233 | 234 | 235 | 236 | 237 | 238 |
| 9 | | | 248 | | 249 | 250 | 251 | | | |
| 10 | | | | | | | | | | |
| 11 | | 271 | 272 | | 2219 | 274 | 275 | 276 | 277 | 278 |
| | | | | | 273 | | | | | |
| 12 | | 288 | | | 289 | 290 | | 291 | 292 | 293 |
| 13 | | 303 | 304 | | | | 305 | | | |
| 14 | | | 315 | | 2220 | 316 | 317 | 318 | 319 | 320 |
| | | | | | | 323 | | 324 | | |
| 15 | | | | | | | | | | |
| 16 | 336 | 337 | 339 | | 340 | 343 | 345 | 346 | 351 | 354 |
| | | 338 | | | 341 | 344 | | 347 | 352 | 355 |
| | | | | | 342 | | | 348 | 353 | 356 |
| | | | | | | | | 349 | | |
| | | | | | | | | 350 | | |
| 17 | | | | | | | 368 | | | |
| 18 | | 380 | 382 | | 384 | 387 | 388 | 389 | 391 | 393 |
| | | 381 | 383 | | 385 | 399 | 400 | 390 | 392 | 394 |
| | | | 397 | | 398 | | | | | |
| | | | | | 386 | | | | | |
| 19 | | | | | | | | | | |
| 20 | | 423 | 424 | | 428 | 432 | 452 | 436 | 440 | 442 |
| | | | 425 | | 429 | 433 | | 437 | 454 | 455 |
| | | | 449 | | 450 | 451 | | 453 | 441 | 443 |
| | | | 426 | | 430 | 434 | | 438 | | |
| | | | 427 | | 431 | 435 | | 439 | | |
| 21 | | | | | | | | | | |
| 22 | | 465 | | | | | | | | |
| 23 | | 468 | 472 | | 475 | | | | | |
| | | 469 | 473 | | | | | | | |
| | | 470 | 474 | | | | | | | |
| | | 471 | | | | | | | | |
| 24 | | | | | | | 482 | | | |
| 25 | | | | | 491 | 492 | | | | |
| 26 | | 499 | 500 | | 501 | | | 502 | 504 | 505 |
| | | | 508 | | 509 | | | 510 | | |
| | | | | | 2226 | | | 503 | | |
| 27 | | | | | | | | | | |
| 28 | | | 515 | | 516 | | | 517 | 520 | 521 |
| | | | | | | | | 518 | | |
| | | | | | | | | 519 | | |
| 29 | | 524 | | | 525 | 526 | | 527 | 528 | 529 |
| 30 | | 532 | | | 533 | | | 534 | 535 | 536 |
| 31 | | 544 | 545 | 547 | 548 | | | | | |
| | | | 550 | | 551 | | | | | |
| | | | 546 | | 2227 | | | | | |
| | | | | | 549 | | | | | |
| 32 | | | | | | | | | | |
| 33 | | | | | | | | | | |
| 34 | | 558 | 559 | | 560 | | | | | |
| 35 | | 2228 | 563 | | 564 | | | 2229 | | |
| 36 | | | 567 | | 569 | | | 570 | | |
| | | | 568 | | | | | 571 | | |
| 37 | | | | | | | | | | |
| 38 | | | | | | | | | | |

Table 1-2

| | | | | | | | | | | |
|---|---|---|---|---|---|---|---|---|---|---|
| 39 | | 584 | | | 585 | | 586 | 587 | | |
| 40 | | | | | | | | | | |
| 41 | | | | 590 | | | | 591 | 2231 | 2232 |
| 42 | | 600 | 601 | 602 | | | 603 | 604 | 605 | 606 |
| 43 | | 612 | 613 | 614 | | | 615 | 616 | 617 | 618 |
| 44 | | | | 621 | 622 | | | 623 | 624 | 625 |
| 45 | | | 633 | 634 | 635 | | | | | |
| 46 | | 637 | 638 | 639 | 640 | | | 641 | 642 | 643 |
| | | | | | | | | 645 | | |
| 47 | | | | | | | | | | |
| 48 | | | | | 650 | | | | | |
| 49 | | | | | | | | | | |
| 50 | | | | | 653 | | | | | |
| 51 | | 671 | 663 | 664 | | | 665 | 666 | 667 | 668 |
| | | | | 672 | | | | 673 | 674 | 675 |
| 52 | | | | | | | | | | |
| 53 | | | | | | | | | | |
| 54 | | | 2235 | 682 | | | | 683 | | |
| 55 | | 686 | 687 | 688 | 689 | | | 690 | 691 | 692 |
| 56 | | | | | | | | | | |
| 57 | | 701 | 702 | 703 | 704 | | 705 | 706 | 707 | 708 |
| | | | | | | | | | | 2237 |
| 58 | | | 714 | | | | | | | |
| 59 | | | 723 | 725 | 726 | | 727 | 728 | 779 | 730 |
| | | | 724 | 2238 | | | | | | |
| 60 | | 733 | | 734 | | | | | | |
| 61 | | 2240 | 738 | 739 | 740 | | | 741 | 742 | 743 |
| 62 | | | | | | | | | | |
| 63 | | | | | | | | | | |
| 64 | | | | | | | | | | |
| 65 | | | 745 | | | | | | | |
| 66 | | | | | | | | | | |
| 67 | | | | | | | | | | |
| 68 | | | | | | | | | | |
| 69 | | | | | 753 | | | | | |
| 70 | | | | | | | | | | |
| 71 | | | | | | | | | | |
| 72 | | | | | 754 | | | | | |
| 73 | | | | | | | | | | |
| 74 | | | | | | | | | | |
| 75 | | | | | | | | | | |
| 76 | | | | | | | | | | |
| 77 | | | | | | | | | | |
| 78 | | | | | | | | | | |
| 79 | | | | | | | | | | |
| 80 | | | | | | | | | | |
| 81 | | | | | 760 | | | | | |
| 82 | | | | | | | | | | |
| 83 | | | | | | | | | | |
| 84 | | | | | | | | | | |
| 85 | | | | | | | | | | |
| 86 | | | | | | | | | | |
| 87 | | | | | | 767 | | | | |
| 88 | | | | | | | | | | |
| 89 | | | | | | | | | | |
| 90 | 773 | 774 | 775 | 776 | | | | | | |
| 91 | | 779 | 780 | 781 | | | | 782 | | |
| 92 | | | | | | | | | | |
| 93 | | | | | | | | | | |
| 94 | | | 785 | 786 | 787 | | | 788 | 789 | 790 |
| 95 | | | | | | | | | | |
| 96 | | | | | | | | | | |
| 97 | | | | | | | | | | |
| 98 | | | 792 | 793 | | | | 794 | 795 | 796 |

Table 1-3

| | | | | | | | | | |
|---|---|---|---|---|---|---|---|---|---|
| 99 | | | | | | | | | |
| 100 | | | | | | | | | |
| 101 | | | | | | | | | |
| 102 | | | | | | | | | |
| 103 | | | | | | | | | |
| 104 | | | | | | | | | |
| 105 | | | | | | | | | |
| 106 | | | | | | | | | |
| 107 | | | | | | | | | |
| 108 | | | | | | | | | |
| 109 | | | | | | | | | |
| 110 | | | | | | | | | |
| 111 | | | | | | | | | |
| 112 | | | | | | | | | |
| 113 | | | | | | | | | |
| 114 | | | | | | | | | |
| 115 | | | | | | | | | |
| 116 | | | | | | | | | |
| 117 | | | | | | | | | |
| 118 | | | | | | | | | |
| 119 | | | | | | | | | |
| 120 | | | | | | | | | |
| 121 | | | | | | | | | |
| 122 | | | | | | | | | |
| 123 | | | | | | | | | |
| 124 | | | | | | | | | |
| 125 | | | | | | | | | |
| 126 | | | | | | | | | |
| 127 | | | | | | | | | |
| 128 | | | | | | | | | |
| 129 | | | | | | | | | |
| 819 | 838 | | 839 | 840 | 842 | 846 | 847 | 850 | 851 |
| | | | | | 843 | | 848 | | |
| 917 | | 773 | 774 | 775 | 776 | | | | |
| 925 | | | | | | | | | |
| 916 | | | | | | | | | |
| 803 | | | | | | | | | |
| 2057 | | | | | | | | | |
| 2058 | | | | | | | | | |
| 2059 | | | | | 2118 | | | | |
| 2060 | | | | | | | | | |
| 2061 | | | | | | | | | |
| 2062 | | | | | | 2119 | | | |
| 2063 | | | | | | | | | |
| 2064 | | | | | | | | | |
| 2065 | | | | | | | | | |
| 2066 | | | | | | | | | |
| 2067 | | | | | | | | | |
| 2068 | | | | | | | | | |
| 2069 | | | | | | | | | |
| 2070 | | | | | | | | | |
| 2071 | | | | | | | | | |
| 2072 | | | | | | | | | |
| 2073 | | | | | | | | | |
| 2074 | | | | | | | | | |
| 2075 | | | | | | | | | |
| 2076 | | | | | | | | | |
| 2077 | | | | | | | | | |
| 2078 | | | | | | | | | |
| 2079 | | | | | | | | | |
| 2080 | | | | | | | | | |
| 2081 | | | | | | | | | |
| 2082 | | | | | | | | | |
| 2083 | | | | | | | | | |
| 2084 | | | | | | | | | |
| 2085 | | | | | | | | | |
| 2086 | | | | | | | | | |
| 2087 | | | | | | | | | |

Table 1-4

2088
2089
2090
2091
2092
2093
2094
2095
2096
2097
2098
2099
2100
2101
2102
2103
2104
2105
2106
2107
2108
2109
2110
2111
2112
2113
2114

Regarding the mechanism in which a miRNA suppresses the translation of the mRNA of a target gene therefor, it is known that a mRNA having a nucleotide sequence complementary to the 2nd to 8th nucleotides from the 5′-end of the miRNA is recognized as a miRNA target gene [Current Biology, 15, R458-R460 (2005)]. By this mechanism, the expression of the mRNA is suppressed by the miRNA. Therefore, miRNAs having the same nucleotide sequence at the 2nd to 8th nucleotides from the 5′-end suppress the expression of the same mRNA to exhibit the same function. As miRNAs having the same nucleotide sequence at the 2nd to 8th nucleotides from the 5′-end as the nucleotide sequence of a miRNA consisting of a nucleotide sequence shown by any one of SEQ ID NO:1-129, 803, 819, 916, 917, 925 and 2057-2114, nucleic acids consisting of a nucleotide sequence shown by any one of SEQ ID NO:804-818, 820-915, 918-924, 926-939 and 2123-2132 can be mentioned. As a specific example of the miRNA consisting of the nucleotide sequence shown by SEQ ID NO:4, the miRNA consisting of the nucleotide sequence shown by SEQ ID NO:804 can be mentioned. Artificial miRNAs are also included in the miRNAs. A table of correspondence of miRNAs having the same nucleotide sequence at the 2nd to 8th nucleotides from the 5′-end as the nucleotide sequence of a miRNA consisting of a nucleotide sequence shown by any one of SEQ ID NO:1-129, 803, 819, 916, 917, 925 and 2057-2114 is shown in Table 2 (from Table 2-1 to Table 2-4). Because miRNAs having a same seed sequence are considered to share the same target nucleotide sequence, they are considered to possess similar functions.

TABLE 2

| | | seed sequence | | | | | | | | | | | | | | | | | | | | | | | | | |
|---|---|---|---|---|---|---|---|---|---|---|---|---|---|---|---|---|---|---|---|---|---|---|---|---|---|---|---|
| SEQ ID NO: | Name of MiRNA | (2-8 nucleotides) | hsa | mmu | mo | cgr | age | lla | sla | mml | mne | ggo | ppa | ptr | ppy | lca | cfa | mdo | gga | xia | xtr | bta | ora | ssc | dre | fra | tni |
| 1 | hsa-miR-1 | GGAAUGU | | | | | | | | | | | | | | | | | | | | | | | | | |
| 2 | hsa-miR-206 | | | | | | | | | | | | | | | | | | | | | | | | | | |
| 803 | hsa-miR-92b | | | | | | | | | | | | | | | | | | | | | | | | | | |
| 3 | hsa-miR-7 | GGAAGAC | | | | | | | | | | | | | | | | | | | | | | | | | |
| 4 | hsa-miR-10a | ACCCUGU | 804 | | | | | | | | | | | | | | | | | | | | | | | | |
| 5 | hsa-mir-18a | AAGGUGC | | | | | | | | | | | | | | | | | | | | | | | | | |
| 6 | hsa-miR-18b | | | | | | | | | | | | | | | | | | | | | | | | | | |
| 7 | hsa-miR-18a* | CUGCCCU | | | | | | | | | | | | | | | | | | | | | | | | | |
| 8 | hsa-miR-20b | AAAGUGC | 805 | 808 | 810 | | | | | | | | | | | | | | | | 812 | 813 | | | 814 | 816 | 817 |
| 9 | hsa-miR-106a | | 806 | 809 | 811 | | | | | | | | | | | | | | | | | | | | | | |
| 10 | hsa-miR-106b | | 807 | | | | | | | | | | | | | | | | | | | | | | | | |
| 11 | hsa-mir-24 | GGCUCAG | | | | | | | | | | | | | | | | | | | | | | | 815 | | |
| 12 | hsa-miR-25 | AUUGCAC | 818 | 822 | 826 | | 829 | 830 | 831 | 832 | 833 | 834 | 835 | 836 | 837 | 838 | | 839 | 840 | | 842 | 846 | | | 847 | 850 | 851 |
| 13 | hsa-miR-32 | | 820 | 823 | 827 | | | | | | | | | | | | | | 841 | | 843 | | | | 848 | | |
| 819 | hsa-miR-92b | | 821 | 824 | 828 | | | | | | | | | | | | | | | | 844 | | | | 849 | | |
| | | | | 825 | | | | | | | | | | | | | | | | | 843 | | | | | | |
| 14 | hsa-miR-26a | UCAAGUA | | | | | | | | | | | | | | | | | | | | | | | | | |
| 15 | hsa-miR-26b | | | | | | | | | | | | | | | | | | | | | | | | | | |
| 16 | hsa-mir-27b | UCACAGU | 852 | | | | | | | | | | | | | | | | | | | | | | | | |
| 17 | hsa-miR-28 | AGGAGCU | 853 | 854 | 855 | | | | | | | | | | | | | | | | | | | | | | |
| 18 | hsa-miR-29a | AGCACCA | 856 | | | | | | | | | | | | | | | | | | | | | | | | |
| 19 | hsa-miR-29c | | | | | | | | | | | | | | | | | | | | | | | | | | |
| 20 | his-miR-30b | GUAAACA | 857 | 860 | 861 | | | | | | | | | | | | | | | | | | | | | | |
| 21 | his-miR-30c | | 858 | | | | | | | | | | | | | | | | | | | | | | | | |
| | | | 859 | | | | | | | | | | | | | | | | | | | | | | | | |
| 22 | hsa-miR-93 | AAGUGCU | 862 | 870 | 873 | | | | | | | | | | | | | | | 874 | 876 | | | | 877 | | |
| 23 | hsa-miR-302d | | 863 | 871 | | | | | | | | | | | | | | | | 875 | | | | | 878 | | |
| | | | 864 | 872 | | | | | | | | | | | | | | | | | | | | | 879 | | |
| | | | 865 | | | | | | | | | | | | | | | | | | | | | | 880 | | |
| | | | 866 | | | | | | | | | | | | | | | | | | | | | | 881 | | |
| | | | 867 | | | | | | | | | | | | | | | | | | | | | | | | |
| | | | 868 | | | | | | | | | | | | | | | | | | | | | | | | |
| | | | 869 | | | | | | | | | | | | | | | | | | | | | | | | |
| 24 | hsa-miR-95 | UCAACGG | | | | | | | | | | | | | | | | | | | | | | | | | |
| 25 | hsa-miR-98 | GAGGUAG | | | | | | | | | | | | | | | | | | | | | | | | | |
| 26 | hsa-miR-130a | AGUGCAA | 882 | 884 | | | | | | | | | | | | | | | | | | | | | 885 | | |
| 27 | hsa-miR-130b | | 883 | | | | | | | | | | | | | | | | | | | | | | 886 | | |
| 28 | hsa-miR301 | | | | | | | | | | | | | | | | | | | | | | | | | | |
| 29 | hsa-miR-132 | AACAGUC | | | | | | | | | | | | | | | | | | | | | | | | | |
| 30 | hsa-miR-212 | | | | | | | | | | | | | | | | | | | | | | | | | | |
| 31 | hsa-miR-133a | UGGUCCC | | | | | | | | | | | | | | | | | | | | | | | | | |
| 32 | hsa-miR-133b | | | | | | | | | | | | | | | | | | | | | | | | | | |
| 33 | hsa-miR-134 | GUGACUG | | | | | | | | | | | | | | | | | | | | | | | | | |
| 34 | hsa-miR-137 | AUUGCUU | | | | | | | | | | | | | | | | | | | | | | | | | |
| 35 | hsa-miR-142-3p | GUAGUGU | | | | | | | | | | | | | | | | | | | | | | | | | |
| 36 | hsa-miR-146a | GAGAACU | 887 | | | | | | | | | | | | | | | | | | | | | | | | |
| 37 | hsa-miR-147 | UGUGUGG | | | | | | | | | | | | | | | | | | | | | | | | | |
| 38 | hsa-miR-149 | CUGGCUC | | | | | | | | | | | | | | | | | | | | | | | | | |
| 39 | hsa-miR-181c | ACAUUCA | | | | | | | | | | | | | | | | | | | | | | | | | |
| 40 | hsa-miR-181d | | | | | | | | | | | | | | | | | | | | | | | | | | |
| 41 | hsa-miR-182* | GGUUCUA | | | | | | | | | | | | | | | | | | | | | | | | | |
| 42 | hsa-miR-183 | AUGGCAC | | | | | | | | | | | | | | | | | | | | | | | | | |
| 43 | hsa-miR-184 | GGACGGA | | | | | | | | | | | | | | | | | | | | | | | | | |
| 44 | hsa-miR-182 | UGACCUA | | | | | | | | | | | | | | | | | | | | | | | | | |
| 45 | hsa-miR-215 | | | | | | | | | | | | | | | | | | | | | | | | | | |
| 46 | hsa-miR-193a | ACUGGCC | | | | | | | | | | | | | | | | | | | | | | | | | |
| 47 | hsa-miR-193b | | | | | | | | | | | | | | | | | | | | | | | | | | |
| 48 | hsa-miR-195 | AGCAGCA | | 888 | 889 | | | | | | | | | | | | | | | | | | | | 890 | | |
| 49 | hsa-miR-424 | | | | | | | | | | | | | | | | | | | | | | | | 891 | | |
| 50 | hsa-miR-497 | | | | | | | | | | | | | | | | | | | | | | | | | | |
| 51 | hsa-miR-196a | AGGUAGU | | | | | | | | | | | | | | | | | | | | | | | | | |
| 52 | hsa-miR-196b | | | | | | | | | | | | | | | | | | | | | | | | | | |
| 53 | hsa-miR-197 | UCACCAC | | | | | | | | | | | | | | | | | | | | | | | | | |
| 54 | hsa-miR-199a* | ACAGUAG | | | | | | | | | | | | | | | | | | | | | | | | | |
| 55 | hsa-miR-200a | AACACUG | 892 | 893 | 894 | | | | | | | | | | | | | 895 | | | | | | | 896 | | |
| 56 | hsa-miR-202 | GAGGUAU | | | | | | | | | | | | | | | | | | | | | | | | | |
| 57 | hsa-miR-204 | UCCCUUU | | | | | | | | | | | | | | | | | | | | | | | | | |
| 58 | hsa-miR-211 | | | | | | | | | | | | | | | | | | | | | | | | | | |
| 59 | hsa-miR-205 | CCUUCAU | | | | | | | | | | | | | | | | | | | | | | | | | |
| 60 | hsa-miR-206 | UAAGACG | 897 | | | | | | | | | | | | | | | | | | | | | | 898 | | |
| 61 | hsa-miR-222 | GCUACAU | 899 | 900 | 901 | | | | | 902 | | 903 | 904 | | 905 | | | 906 | 907 | | 908 | 909 | | | 910 | 911 | 912 |
| 62 | hsa-miR-299-3p | AUGUGGG | | | | | | | | | | | | | | | | | | | | | | | | | |
| 63 | hsa-miR-299-5p | GGUUUAC | | | | | | | | | | | | | | | | | | | | | | | | | |
| 64 | hsa-miR-302a* | AAACGUG | | | | | | | | | | | | | | | | | | | | | | | | | |
| 65 | hsa-miR-302b* | CUUUAAC | 913 | | | | | | | | | | | | | | | | | | | | | | | | |
| 66 | hsa-miR-324-3p | CACUGCC | | | | | | | | | | | | | | | | | | | | | | | | | |
| 67 | hsa-miR-324-5p | GCAUCCC | | | | | | | | | | | | | | | | | | | | | | | | | |
| 68 | hsa-miR-329 | ACACACC | 914 | 915 | | | | | | | | | | | | | | | | | | | | | | | |
| 69 | hsa-miR-331 | CCCCUGG | | | | | | | | | | | | | | | | | | | | | | | | | |
| 70 | hsa-miR-337 | CCAGCUC | | | | | | | | | | | | | | | | | | | | | | | | | |
| 71 | hsa-miR-340 | CCGUCUC | | | | | | | | | | | | | | | | | | | | | | | | | |
| 72 | hsa-miR-342 | CUCACAC | | | | | | | | | | | | | | | | | | | | | | | | | |
| 73 | hsa-miR-346 | GUCUGCC | | | | | | | | | | | | | | | | | | | | | | | | | |
| 74 | hsa-miR-362 | AUCCUUG | | | | | | | | | | | | | | | | | | | | | | | | | |
| 75 | hsa-miR-368 | CAUAGAG | | | | | | | | | | | | | | | | | | | | | | | | | |
| 76 | hsa-miR-371 | UGCCGCC | | | | | | | | | | | | | | | | | | | | | | | | | |
| 77 | hsa-miR-376a | UCAUAGA | | | | | | | | | | | | | | | | | | | | | | | | | |
| 78 | hsa-miR-376b | | | | | | | | | | | | | | | | | | | | | | | | | | |
| 79 | hsa-miR-377 | UCACACA | | | | | | | | | | | | | | | | | | | | | | | | | |
| 80 | hsa-miR-378 | UCCUGAC | | | | | | | | | | | | | | | | | | | | | | | | | |
| 81 | hsa-miR-380-5p | GGUUGAC | | | | | | | | | | | | | | | | | | | | | | | | | |
| 916 | hsa-miR-583 | | | | | | | | | | | | | | | | | | | | | | | | | | |
| 82 | hsa-miR-382 | AAGUUGU | | | | | | | | | | | | | | | | | | | | | | | | | |
| 83 | hsa-miR-412 | CUUCACC | | | | | | | | | | | | | | | | | | | | | | | | | |
| 84 | hsa-miR-422a | UGGACUU | | | | | | | | | | | | | | | | | | | | | | | | | |
| 85 | hsa-miR-422b | | | | | | | | | | | | | | | | | | | | | | | | | | |
| 86 | hsa-miR-423 | GCUCGGU | | | | | | | | | | | | | | | | | | | | | | | | | |
| 87 | hsa-miR-431 | GUCUUGC | | | | | | | | | | | | | | | | | | | | | | | | | |
| 88 | hsa-miR-432* | UGGAUGG | | | | | | | | | | | | | | | | | | | | | | | | | |
| 89 | hsa-miR-433 | UCAUGAU | | | | | | | | | | | | | | | | | | | | | | | | | |
| 90 | hsa-miR-449 | GGCAGUG | | 918 | | | | | | | | | | | | | | | | | | | | | | | |
| 917 | hsa-miR-449b | | | | | | | | | | | | | | | | | | | | | | | | | | |
| 91 | hsa-miR-451 | AACCGUU | | | | | | | | | | | | | | | | | | | | | | | | | |
| 92 | hsa-miR-452 | GUUUGCA | | | | | | | | | | | | | | | | | | | | | | | | | |
| 93 | hsa-miR-452* | CAGUCUC | | | | | | | | | | | | | | | | | | | | | | | | | |
| 94 | hsa-miR-455 | AUGUGCC | | | | | | | | | | | | | | | | | | | | | | | | | |
| 95 | hsa-miR-483 | CACUCCU | | | | | | | | | | | | | | | | | | | | | | | | | |
| 96 | hsa-miR-485-5p | GAGGCUG | | | | | | | | | | | | | | | | | | | | | | | | | |
| 97 | hsa-miR-488 | CCAGAUA | | | | | | | | | | | | | | | | | | | | | | | | | |
| 98 | hsa-miR-489 | GUGACAU | | | | | | | | | | | | | | | | | | | | | | | | | |
| 99 | hsa-miR-491 | GUGGGGA | | | | | | | | | | | | | | | | | | | | | | | | | |
| 100 | hsa-miR-493-5p | UGUACAU | | | | | | | | | | | | | | | | | | | | | | | | | |
| 101 | hsa-miR-494 | GAAACAU | | | | | | | | | | | | | | | | | | | | | | | | | |
| 102 | hsa-miR-501 | AUCCUUU | | | | | | | | | | | | | | | | | | | | | | | | | |
| 103 | hsa-miR-504 | GACCCUG | | | | | | | | | | | | | | | | | | | | | | | | | |
| 104 | hsa-miR-505 | UCAACAC | | | | | | | | | | | | | | | | | | | | | | | | | |
| 105 | hsa-miR-506 | AAGGCAC | 919 | 920 | 921 | | | | | | | | | | | | | | | | | | | | 922 | 923 | 924 |
| 106 | hsa-miR-507 | UUUGCAC | | | | | | | | | | | | | | | | | | | | | | | | | |
| 925 | hsa-miR-557 | | | | | | | | | | | | | | | | | | | | | | | | | | |
| 107 | hsa-miR-509 | GAUUGGU | | | | | | | | | | | | | | | | | | | | | | | | | |
| 108 | hsa-miR-510 | ACUCAGG | | | | | | | | | | | | | | | | | | | | | | | | | |
| 109 | hsa-miR-511 | UGUCUUU | | | | | | | | | | | | | | | | | | | | | | | | | |
| 110 | hsa-miR-512-3p | AGUGCUG | | | | | | | | | | | | | | | | | | | | | | | | | |
| 111 | hsa-miR-512-5p | ACUCAGC | | | | | | | | | | | | | | | | | | | | | | | | | |
| 112 | hsa-miR-513 | UCACAGG | | | | | | | | | | | | | | | | | | | | | | | | | |
| 113 | hsa-miR-516-5p | AUCUGGA | | | | | | | | | | | | | | | | | | | | | | | | | |
| 114 | hsa-miR-517* | CUCUAGA | | | | | | | | | | | | | | | | | | | | | | | | | |
| 115 | hsa-miR-517a | UCGUGCA | | | | | | | | | | | | | | | | | | | | | | | | | |
| 116 | hsa-miR-517c | | | | | | | | | | | | | | | | | | | | | | | | | | |
| 117 | hsa-miR-517b | CGUGCAU | | | | | | | | | | | | | | | | | | | | | | | | | |
| 118 | hsa-miR-518b | AAAGCGC | 925 | | | | | | | | | | | | | | | | | | | | | | | | |
| 119 | hsa-miR-518c | | | | | | | | | | | | | | | | | | | | | | | | | | |
| 120 | hsa-miR-518d | | | | | | | | | | | | | | | | | | | | | | | | | | |
| 121 | hsa-miR-518c* | CUCUGGA | | | | | | | | | | | | | | | | | | | | | | | | | |
| 122 | hsa-miR-518e | AAGCGCU | | | | | | | | | | | | | | | | | | | | | | | | | |
| 123 | hsa-miR-518f | | | | | | | | | | | | | | | | | | | | | | | | | | |
| 124 | hsa-miR-520h | CAAAGUG | 927 | | | | | | | | | | | | | | | | | | | | | | | | |
| 125 | hsa-miR-523 | ACGCGCU | | | | | | | | | | | | | | | | | | | | | | | | | |
| 126 | hsa-miR-524* | UACAAAG | 928 | | | | | | | | | | | | | | | | | | | | | | | | |
| 127 | hsa-miR-525 | UCCAGAG | 929 | | | | | | | | | | | | | | | | | | | | | | | | |
| 128 | hsa-miR-526a | UCUAGAG | 930 | | | | | | | | | | | | | | | | | | | | | | | | |
| | | | 931 | | | | | | | | | | | | | | | | | | | | | | | | |
| | | | 932 | | | | | | | | | | | | | | | | | | | | | | | | |
| | | | 933 | | | | | | | | | | | | | | | | | | | | | | | | |
| | | | 934 | | | | | | | | | | | | | | | | | | | | | | | | |
| | | | 935 | | | | | | | | | | | | | | | | | | | | | | | | |
| | | | 936 | | | | | | | | | | | | | | | | | | | | | | | | |
| | | | 937 | | | | | | | | | | | | | | | | | | | | | | | | |
| | | | 938 | | | | | | | | | | | | | | | | | | | | | | | | |
| 129 | hsa-miR-527 | UGCAAAG | 939 | | | | | | | | | | | | | | | | | | | | | | | | |
| 2057 | hsa-miR-411 | AGUAGAC | | | | | | | | | | | | | | | | | | | | | | | | | |
| 2058 | hsa-miR-421 | UCAACAG | | | | | | | | | | | | | | | | | | | | | | | | | |
| 2059 | hsa-miR-425-5p | AUGACAC | | | | | | | | | | | | | | | | | | | | | | | | | |
| 2060 | hsa-miR-454-5p | CCCUAUC | | | | | | | | | | | | | | | | | | | | | | | | | |
| 2061 | hsa-miR-493-3p | GAAGGUC | | 2123 | | | | | | | | | | | | | | | | | | | | | 2124 | | |
| 2062 | hsa-miR-532 | AUGCCUU | | | | | | | | | | | | | | | | | | | | | | | | | |
| 2063 | hsa-miR-542-3p | GUGACAG | 2125 | | | | | | | | | | | | | | | | | | | | | | | | |
| 2064 | hsa-miR-545 | UCAGCAA | | | | | | | | | | | | | | | | | | | | | | | | | |
| 2065 | hsa-miR-548a | AAAACUG | | | | | | | | | | | | | | | | | | | | | | | | | |
| 2066 | hsa-miR-548b | AAGAACC | | | | | | | | | | | | | | | | | | | | | | | | | |
| 2067 | hsa-miR-548d | AAAAACC | | | | | | | | | | | | | | | | | | | | | | | | | |
| 2068 | hsa-miR-550 | GUCUUAC | 2126 | 2127 | | | | | | | | | | | | | | | | | | | | | | | |
| 2069 | hsa-miR-551b | CGACCCA | 2128 | | | | | | | | | | | | | | | | | | | | | | | | |
| 2070 | hsa-miR-552 | ACAGGUG | | | | | | | | | | | | | | | | | | | | | | | | | |
| 2071 | hsa-miR-553 | AAACGGU | | | | | | | | | | | | | | | | | | | | | | | | | |
| 2072 | hsa-miR-558 | GAGCUGC | | | | | | | | | | | | | | | | | | | | | | | | | |
| 2073 | hsa-miR-561 | AAAGUUU | | | | | | | | | | | | | | | | | | | | | | | | | |
| 2074 | hsa-miR-565 | GCUGGCU | | | | | | | | | | | | | | | | | | | | | | | | | |
| 2075 | hsa-miR-570 | AAAACAG | | | | | | | | | | | | | | | | | | | | | | | | | |
| 2076 | hsa-miR-571 | GAGUUGG | | | | | | | | | | | | | | | | | | | | | | | | | |
| 2077 | hsa-miR-579 | UUCAUUU | | | | | | | | | | | | | | | | | | | | | | | | | |
| 2078 | hsa-miR-584 | UAUGGUU | | | | | | | | | | | | | | | | | | | | | | | | | |
| 2079 | hsa-miR-589 | CAGAACA | | | | | | | | | | | | | | | | | | | | | | | | | |
| 2080 | hsa-miR-591 | GACCAUG | | | | | | | | | | | | | | | | | | | | | | | | | |
| 2081 | hsa-miR-593 | GGCACCA | | | | | | | | | | | | | | | | | | | | | | | | | |
| 2032 | hsa-miR-595 | AAGUGUG | | | | | | | | | | | | | | | | | | | | | | | | | |
| 2083 | hsa-miR-599 | UUGUGUC | | 2129 | | | | | | | | | | | | | | | | | | | | | | | |
| 2084 | hsa-miR-600 | CUUACAG | | | | | | | | | | | | | | | | | | | | | | | | | |
| 2085 | hsa-miR-601 | GGUCUAG | | | | | | | | | | | | | | | | | | | | | | | | | |
| 2086 | hsa-miR-604 | GGCUGCG | | | | | | | | | | | | | | | | | | | | | | | | | |
| 2087 | hsa-miR-605 | AAAUCCC | | | | | | | | | | | | | | | | | | | | | | | | | |
| 2088 | hsa-miR-606 | AACUACU | | | | | | | | | | | | | | | | | | | | | | | | | |
| 2089 | hsa-miR-609 | GGGUGUU | | | | | | | | | | | | | | | | | | | | | | | | | |
| 2090 | hsa-miR-610 | GAGCUAA | | | | | | | | | | | | | | | | | | | | | | | | | |
| 2091 | hsa-miR-611 | CGAGGAC | | | | | | | | | | | | | | | | | | | | | | | | | |
| 2092 | hsa-miR-614 | AACGCCU | | | | | | | | | | | | | | | | | | | | | | | | | |
| 2093 | hsa-miR-615 | CCGAGCC | | | | | | | | | | | | | | | | | | | | | | | | | |
| 2094 | hsa-miR-616 | CUCAAAA | 2130 | 2131 | | | | | | | | | | | | | | | | | | | | | | | |
| 2095 | hsa-miR-617 | GACUUCC | | | | | | | | | | | | | | | | | | | | | | | | | |
| 2096 | hsa-miR-618 | AACUCUA | | | | | | | | | | | | | | | | | | | | | | | | | |
| 2097 | hsa-miR-621 | GCUAGCA | | | | | | | | | | | | | | | | | | | | | | | | | |
| 2098 | hsa-miR-624 | AGUACCA | | | | | | | | | | | | | | | | | | | | | | | | | |
| 2099 | hsa-miR-625 | GGGGGAA | | | | | | | | | | | | | | | | | | | | | | | | | |
| 2100 | hsa-miR-627 | UGAGUCU | | | | | | | | | | | | | | | | | | | | | | | | | |
| 2101 | hsa-miR-630 | GUAUUCU | | | | | | | | | | | | | | | | | | | | | | | | | |
| 2102 | hsa-miR-631 | GACCUGG | | | | | | | | | | | | | | | | | | | | | | | | | |
| 2103 | hsa-miR-634 | ACCAGCA | | | | | | | | | | | | | | | | | | | | | | | | | |
| 2104 | hsa-miR-637 | CUGGGGG | | | | | | | | | | | | | | | | | | | | | | | | | |
| 2105 | hsa-miR-646 | AGCAGCU | | | | | | | | | | | | | | | | | | | | | | | | | |
| 2106 | hsa-miR-647 | UGGCUGC | | | | | | | | | | | | | | | | | | | | | | | | | |
| 2107 | hsa-miR-648 | AGUGUGC | | | | | | | | | | | | | | | | | | | | | | | | | |
| 2108 | hsa-miR-650 | GGAGGCA | | | | | | | | | | | | | | | | | | | | | | | | | |
| 2109 | hsa-miR-651 | UUAGGAU | | | | | | | | | | | | | | | | | | | | | | | | | |
| 2110 | hsa-miR-653 | UGAAACA | | | | | | | | | | | | | | | | | | | | | | | | | |
| 2111 | hsa-miR-654 | GGUGGGC | 2132 | | | | | | | | | | | | | | | | | | | | | | | | |
| 2112 | hsa-miR-657 | GCAGGUU | | | | | | | | | | | | | | | | | | | | | | | | | |
| 2113 | hsa-miR-661 | GCCUGGG | | | | | | | | | | | | | | | | | | | | | | | | | |
| 2114 | hsa-miR-759-5p | GAGACCU | | | | | | | | | | | | | | | | | | | | | | | | | |

As the above-described nucleic acids, miRNA precursors can also be used preferably. A miRNA precursor is a nucleic 5 acid about 50 to about 200 nucleotides long, more preferably about 70 to about 100 nucleotides long, including the above-described nucleic acid, and capable of forming a hairpin structure. A miRNA is produced from a miRNA precursor via processing by a protein called Dicer.

As examples of miRNA precursors for the human miRNAs of SEQ ID NO:1, nucleic acids consisting of a nucleotide sequence shown by SEQ ID NO:940 or 941 can be mentioned. For the miRNAs consisting of a nucleotide sequence shown by any one of SEQ ID NO:2-939, 2057-2132, 2212-2261 and 2315-2321, nucleic acids consisting of a nucleotide sequence shown by any one of SEQ ID NO:942-2056, 2133-2211, 2262-2314 and 2322-2328 can be mentioned. A table of correspondence of miRNAs and miRNA precursors in the present invention is shown in Table 3 (from Table 3-1 to Table 3-17). In the present invention, miRNA precursors include artificial miRNA precursors.

| | | | |
|---|---|---|---|
| | | | Name of |
| SEQ ID NO: | Name of miRNA | SEQ ID NO: | miRNA precursor |

Table 3-1

| | | | |
|---|---|---|---|
| 1 | hsa-miR-1 | 940 | hsa-mir-1-1 |
| | | 941 | hsa-mir-1-2 |
| 2 | hsa-miR-206 | 942 | hsa-mir-206 |
| 3 | hsa-miR-7 | 943 | hsa-mir-7-1 |
| | | 944 | hsa-mir-7-2 |
| | | 945 | hsa-mir-7-3 |
| 4 | hsa-miR-10a | 946 | hsa-mir-10a |
| 5 | hsa-miR-18a | 947 | hsa-mir-18a |
| 6 | hsa-miR-18b | 948 | hsa-mir-18b |
| 7 | hsa-miR-18a* | 947 | hsa-mir-18a |
| 8 | hsa-miR-20b | 949 | hsa-mir-20b |
| 9 | hsa-miR-106a | 950 | hsa-mir-106a |
| 10 | hsa-miR-106b | 951 | hsa-mir-106b |
| 11 | hsa-miR-24 | 952 | hsa-mir-24-1 |
| | | 953 | hsa-mir-24-2 |
| 12 | hsa-miR-25 | 954 | hsa-mir-25 |
| 13 | hsa-miR-32 | 955 | hsa-mir-32 |
| 14 | hsa-miR-26a | 956 | hsa-mir-26a-1 |
| | | 957 | hsa-mir-26a-2 |
| 15 | hsa-miR-26b | 958 | hsa-mir-26b |
| 16 | hsa-miR-27b | 959 | hsa-mir-27b |
| 17 | hsa-miR-28 | 960 | hsa-mir-28 |
| 18 | hsa-miR-29a | 961 | hsa-mir-29a |
| 19 | hsa-miR-29c | 962 | hsa-mir-29c |
| 20 | hsa-miR-30b | 963 | hsa-mir-30b |
| 21 | hsa-miR-30c | 964 | hsa-mir-30c-1 |
| | | 965 | hsa-mir-30c-2 |
| 22 | hsa-miR-93 | 966 | hsa-mir-93 |
| 23 | hsa-miR-302d | 967 | hsa-mir-302d |
| 24 | hsa-miR-95 | 968 | hsa-mir-95 |
| 25 | hsa-miR-98 | 969 | hsa-mir-98 |
| 26 | hsa-miR-130a | 970 | hsa-mir-130a |
| 27 | hsa-miR-130b | 971 | hsa-mir-130b |
| 28 | hsa-miR-301 | 972 | hsa-mir-301 |
| 29 | hsa-miR-132 | 973 | hsa-mir-132 |
| 30 | hsa-miR-212 | 974 | hsa-mir-212 |
| 31 | hsa-miR-133a | 975 | hsa-mir-133a-1 |
| | | 976 | hsa-mir-133a-2 |
| 32 | hsa-miR-133b | 977 | hsa-mir-133b |
| 33 | hsa-miR-134 | 978 | hsa-mir-134 |
| 34 | hsa-miR-137 | 979 | hsa-mir-137 |
| 35 | hsa-miR-142-3p | 980 | hsa-mir-142 |
| 36 | hsa-miR-146a | 981 | hsa-mir-146a |
| 37 | hsa-miR-147 | 982 | hsa-mir-147 |
| 38 | hsa-miR-149 | 983 | hsa-mir-149 |
| 39 | hsa-miR-181c | 984 | hsa-mir-181c |
| 40 | hsa-miR-181d | 985 | hsa-mir-181d |
| 41 | hsa-miR-182* | 986 | hsa-mir-182 |
| 42 | hsa-miR-183 | 987 | hsa-mir-183 |
| 43 | hsa-miR-184 | 988 | hsa-mir-184 |
| 44 | hsa-miR-192 | 989 | hsa-mir-192 |
| 45 | hsa-miR-215 | 990 | hsa-mir-215 |
| 46 | hsa-miR-193a | 991 | hsa-mir-193a |
| 47 | hsa-miR-193b | 992 | hsa-mir-193b |
| 48 | hsa-miR-195 | 993 | hsa-mir-195 |
| 49 | hsa-miR-424 | 994 | hsa-mir-424 |
| 50 | hsa-miR-497 | 995 | hsa-mir-497 |
| 51 | hsa-miR-196a | 996 | hsa-mir-196a-1 |
| | | 997 | hsa-mir-196a-2 |
| 52 | hsa-miR-196b | 998 | hsa-mir-196b |
| 53 | hsa-miR-197 | 999 | hsa-mir-197 |
| 54 | hsa-miR-199a* | 1000 | hsa-mir-199a-1 |
| | | 1001 | hsa-mir-199a-2 |
| 55 | hsa-miR-200a | 1002 | hsa-mir-200a |
| 56 | hsa-miR-202 | 1003 | hsa-mir-202 |
| 57 | hsa-miR-204 | 1004 | hsa-mir-204 |
| 58 | hsa-miR-211 | 1005 | hsa-mir-211 |
| 59 | hsa-miR-205 | 1006 | hsa-mir-205 |
| 60 | hsa-miR-208 | 1007 | hsa-mir-208 |
| 61 | hsa-miR-222 | 1008 | hsa-mir-222 |
| 62 | hsa-miR-299-3p | 1009 | hsa-mir-299 |
| 63 | hsa-miR-299-5p | 1009 | hsa-mir-299 |
| 64 | hsa-miR-302a* | 1010 | hsa-mir-302a |
| 65 | hsa-miR-302b* | 1011 | hsa-mir-302b |
| 66 | hsa-miR-324-3p | 1012 | hsa-mir-324 |
| 67 | hsa-miR-324-5p | 1012 | hsa-mir-324 |
| 68 | hsa-miR-329 | 1013 | hsa-mir-329-1 |
| | | 1014 | hsa-mir-329-2 |
| 69 | hsa-miR-331 | 1015 | hsa-mir-331 |
| 70 | hsa-miR-337 | 1016 | hsa-mir-337 |
| 71 | hsa-miR-340 | 1017 | hsa-mir-340 |
| 72 | hsa-miR-342 | 1018 | hsa-mir-342 |

Table 3-2

| | | | |
|---|---|---|---|
| 73 | hsa-miR-346 | 1019 | hsa-mir-346 |
| 74 | hsa-miR-362 | 1020 | hsa-mir-362 |
| 75 | hsa-miR-368 | 1021 | hsa-mir-368 |
| 76 | hsa-miR-371 | 1022 | hsa-mir-371 |
| 77 | hsa-miR-376a | 1023 | hsa-mir-376a-1 |
| | | 1024 | hsa-mir-376a-2 |
| 78 | hsa-miR-376b | 1025 | hsa-mir-376b |
| 79 | hsa-miR-377 | 1026 | hsa-mir-377 |
| 80 | hsa-miR-378 | 1027 | hsa-mir-378 |
| 81 | hsa-miR-380-5p | 1028 | hsa-mir-380 |
| 82 | hsa-miR-382 | 1029 | hsa-mir-382 |
| 83 | hsa-miR-412 | 1030 | hsa-mir-412 |
| 84 | hsa-miR-422a | 1031 | hsa-mir-422a |
| 85 | hsa-miR-422b | 1027 | hsa-mir-378 |
| 86 | hsa-miR-423 | 1032 | hsa-mir-423 |
| 87 | hsa-miR-431 | 1033 | hsa-mir-431 |
| 88 | hsa-miR-432* | 1034 | hsa-mir-432 |
| 89 | hsa-miR-433 | 1035 | hsa-mir-433 |
| 90 | hsa-miR-449 | 1036 | hsa-mir-449 |
| 91 | hsa-miR-451 | 1037 | hsa-mir-451 |
| 92 | hsa-miR-452 | 1038 | hsa-mir-452 |
| 93 | hsa-miR-452* | 1038 | hsa-mir-452 |
| 94 | hsa-miR-455 | 1039 | hsa-mir-455 |
| 95 | hsa-miR-483 | 1040 | hsa-mir-483 |
| 96 | hsa-miR-485-5p | 1041 | hsa-mir-485 |
| 97 | hsa-miR-488 | 1042 | hsa-mir-488 |
| 98 | hsa-miR-489 | 1043 | hsa-mir-489 |
| 99 | hsa-miR-491 | 1044 | hsa-mir-491 |
| 100 | hsa-miR-493-5p | 1045 | hsa-mir-493 |
| 101 | hsa-miR-494 | 1046 | hsa-mir-494 |
| 102 | hsa-miR-501 | 1047 | hsa-mir-501 |
| 103 | hsa-miR-504 | 1048 | hsa-mir-504 |
| 104 | hsa-miR-505 | 1049 | hsa-mir-505 |
| 105 | hsa-miR-506 | 1050 | hsa-mir-506 |
| 106 | hsa-miR-507 | 1051 | hsa-mir-507 |
| 107 | hsa-miR-509 | 1052 | hsa-mir-509 |
| 108 | hsa-miR-510 | 1053 | hsa-mir-510 |
| 109 | hsa-miR-511 | 1054 | hsa-mir-511-1 |
| | | 1055 | hsa-mir-511-2 |
| 110 | hsa-miR-512-3p | 1056 | hsa-mir-512-1 |
| | | 1057 | hsa-mir-512-2 |
| 111 | hsa-miR-512-5p | 1056 | hsa-mir-512-1 |
| | | 1057 | hsa-mir-512-2 |
| 112 | hsa-miR-513 | 1058 | hsa-mir-513-1 |
| | | 1059 | hsa-mir-513-2 |
| 113 | hsa-miR-516-5p | 1060 | hsa-mir-516-1 |
| | | 1061 | hsa-mir-516-2 |
| | | 1062 | hsa-mir-516-3 |
| | | 1063 | hsa-mir-516-4 |
| 114 | hsa-miR-517* | 1064 | hsa-mir-517a |
| | | 1065 | hsa-mir-517b |
| | | 1066 | hsa-mir-517c |
| 115 | hsa-miR-517a | 1064 | hsa-mir-517a |
| 116 | hsa-miR-517c | 1066 | hsa-mir-517c |
| 117 | hsa-miR-517b | 1065 | hsa-mir-517b |
| 118 | hsa-miR-518b | 1067 | hsa-mir-518b |
| 119 | hsa-miR-518c | 1068 | hsa-mir-518c |
| 120 | hsa-miR-518d | 1069 | hsa-mir-518d |
| 121 | hsa-miR-518o* | 1068 | hsa-mir-518c |
| 122 | hsa-miR-518e | 1070 | hsa-mir-518e |
| 123 | hsa-miR-518f | 1071 | hsa-mir-518f |
| 124 | hsa-miR-520h | 1072 | hsa-mir-520h |
| 125 | hsa-miR-523 | 1073 | hsa-mir-523 |
| 126 | hsa-miR-524* | 1074 | hsa-mir-524 |
| 127 | hsa-miR-525 | 1075 | hsa-mir-525 |
| 128 | hsa-miR-526a | 1076 | hsa-mir-526a-1 |
| | | 1077 | hsa-mir-526a-2 |
| 129 | hsa-mir-527 | 1078 | hsa-mir-527 |

Table 3-3

| | | | |
|---|---|---|---|
| 130 | mmu-miR-1 | 1079 | mmu-mir-1-1 |
| | | 1080 | mmu-mir-1-2 |
| 131 | rno-miR-1 | 1081 | rno-mir-1 |
| 132 | ppa-miR-1 | 1082 | ppa-mir-1 |
| 133 | mdo-miR-1 | 1083 | mdo-mir-1 |
| 134 | gga-miR-1a | 1084 | gga-mir-1a-1 |
| | | 1085 | gga-mir-1a-2 |
| 135 | gga-miR-1b | 1086 | gga-mir-1b |
| 136 | xtr-miR-1b | 1087 | xtr-mir-1b |
| 137 | dre-miR-1 | 1088 | dre-mir-1-1 |
| | | 1089 | dre-mir-1-2 |
| 138 | fru-miR-1 | 1090 | fru-mir-1 |
| 139 | tni-miR-1 | 1091 | tni-mir-1 |
| 140 | mmu-miR-206 | 1092 | mmu-mir-206 |
| 141 | rno-miR-206 | 1093 | rno-mir-206 |
| 142 | mne-miR-206 | 1094 | mne-mir-206 |
| 143 | ppy-miR-206 | 1095 | ppy-mir-206 |
| 144 | mdo-miR-206 | 1096 | mdo-mir-206 |
| 145 | gga-miR-206 | 1097 | gga-mir-206 |
| 146 | xtr-miR-206 | 1098 | xtr-mir-206 |
| 147 | dre-miR-206 | 1099 | dre-mir-206-1 |
| | | 1100 | dre-mir-206-2 |
| 148 | mmu-miR-7b | 1101 | mmu-mir-7b |
| 149 | rno-miR-7a | 1102 | rno-mir-7a-1 |
| | | 1103 | rno-mir-7a-2 |
| 150 | rno-miR-7b | 1104 | rno-mir-7b |
| 151 | lla-miR-7 | 1105 | lla-mir-7 |
| 152 | sla-miR-7 | 1106 | sla-mir-7 |
| 153 | mne-miR-7 | 1107 | mne-mir-7-1 |
| | | 1108 | mne-mir-7-2 |
| 154 | ggo-miR-7 | 1109 | ggo-mir-7-1 |
| | | 1110 | ggo-mir-7-2 |
| | | 1111 | ggo-mir-7-3 |
| 155 | ppa-miR-7 | 1112 | ppa-mir-7-1 |
| | | 1113 | ppa-mir-7-2 |
| | | 1114 | ppa-mir-7-3 |
| 156 | ptr-miR-7 | 1115 | ptr-mir-7 |
| 157 | ppy-miR-7 | 1116 | ppy-mir-7-1 |
| | | 1117 | ppy-mir-7-2 |
| 158 | mdo-miR-7 | 1118 | mdo-mir-7 |
| 159 | gga-miR-7 | 1119 | gga-mir-7-1 |
| | | 1120 | gga-mir-7-2 |
| | | 1121 | gga-mir-7-3 |
| 160 | gga-miR-7b | 1122 | gga-mir-7b |
| 161 | xtr-miR-7 | 1123 | xtr-mir-7-1 |
| | | 1124 | xtx-mir-7-2 |
| | | 1125 | xtr-mir-7-3 |
| 162 | bta-miR-7 | 1126 | bta-mir-7 |
| 163 | ssc-miR-7 | 1127 | ssc-mir-7 |
| 164 | dre-miR-7a | 1128 | dre-mir-7a-1 |
| | | 1129 | dre-mir-7a-2 |
| | | 1130 | dre-mir-7a-3 |
| 165 | dre-miR-7b | 1131 | dre-mir-7b |
| 166 | fru-miR-7 | 1132 | fru-mir-7 |
| 167 | tni-miR-7 | 1133 | tni-mir-7 |
| 168 | mmu-miR-10a | 1134 | mmu-mir-10a |
| 169 | age-miR-10a | 1135 | age-mir-10a |
| 170 | sla-miR-10a | 1136 | sla-mir-10a |
| 171 | mne-miR-10b | 1137 | mne-mir-10b |
| 172 | ggo-miR-10a | 1138 | ggo-mir-10a |
| 173 | ggo-miR-10b | 1139 | ggo-mir-10b |
| 174 | ppa-miR-10a | 1140 | ppa-mir-10a |
| 175 | ppa-miR-10b | 1141 | ppa-mir-10b |
| 176 | mdo-miR-10a | 1142 | mdo-mir-10a |
| 177 | gga-miR-10b | 1143 | gga-mir-10b |
| 178 | xtr-miR-10a | 1144 | xtr-mir-10a |
| 179 | xtr-miR-10b | 1145 | xtr-mir-10b |
| 180 | xtr-miR-10c | 1146 | xtr-mir-10c |
| 181 | bta-miR-10a | 1147 | bta-mir-10a |
| 182 | bta-miR-10b | 1148 | bta-mir-10b |
| 183 | dre-miR-10a | 1149 | dre-mir-10a |
| 184 | dre-miR-10b | 1150 | dre-mir-10b-1 |
| | | 1151 | dre-mir-10b-2 |
| 185 | dre-miR-10c | 1152 | dre-mir-10c |
| 186 | dre-miR-10d | 1153 | dre-mir-10d-1 |
| | | 1154 | dre-mir-10d-2 |

Table 3-4

| | | | |
|---|---|---|---|
| 187 | fru-miR-10b | 1155 | fru-mir-10b-1 |
| | | 1156 | fru-mir-10b-2 |
| 188 | fru-miR-10c | 1157 | fru-mir-10c |
| 189 | fru-miR-10d | 1158 | fru-mir-10d |
| 190 | tni-miR-10b | 1159 | tni-mir-10b-1 |
| | | 1160 | tni-mir-10b-2 |
| 191 | tni-miR-10c | 1161 | tni-mir-10c |
| 192 | tni-miR-10d | 1162 | tni-mir-10d |
| 193 | mmu-miR-18a | 1163 | mmu-mir-18a |
| 194 | rno-miR-18a | 1164 | rno-mir-18a |
| 195 | age-miR-18 | 1165 | age-mir-18 |
| 196 | lla-miR-18 | 1166 | lla-mir-18 |
| 197 | sla-miR-18 | 1167 | sla-mir-18 |
| 198 | mml-miR-18 | 1168 | mml-mir-18 |
| 199 | mne-miR-18 | 1169 | mne-mir-18 |
| 200 | ggo-miR-18 | 1170 | ggo-mir-18 |
| 201 | ppa-miR-18 | 1171 | ppa-mir-18 |
| 202 | ptr-miR-18 | 1172 | ptr-mir-18 |
| 203 | ppy-miR-18 | 1173 | ppy-mir-18 |
| 204 | lca-miR-18 | 1174 | lca-mir-18 |
| 205 | mdo-miR-18 | 1175 | mdo-mir-18 |
| 206 | gga-miR-18a | 1176 | gga-mir-18a |
| 207 | xtr-miR-18a | 1177 | xtr-mir-18a |
| 208 | bta-miR-18a | 1178 | bta-mir-18a |
| 209 | ssc-miR-18 | 1179 | ssc-mir-18 |
| 210 | dre-miR-18a | 1180 | dre-mir-18a |
| 211 | dre-miR-18c | 1181 | dre-mir-18c |
| 212 | fru-miR-18 | 1182 | fru-mir-18 |
| 213 | tni-miR-18 | 1183 | tni-mir-18 |
| 214 | mmu-miR-18b | 1184 | mmu-mir-18b |
| 215 | gga-miR-18b | 1185 | gga-mir-18b |
| 216 | xtr-miR-18b | 1186 | xtr-mir-18b |
| 217 | bta-miR-18b | 1187 | bta-mir-18b |
| 218 | dre-miR-18b | 1188 | dre-mir-18b |
| 219 | mmu-miR-18a* | 1163 | mmu-mir-18a |
| 220 | mmu-miR-20b | 1189 | mmu-mir-20b |
| 221 | age-miR-20 | 1190 | age-mir-20 |
| 222 | lla-miR-20 | 1191 | lla-mir-20 |
| 223 | sla-miR-20 | 1192 | sla-mir-20 |
| 224 | mml-miR-20 | 1193 | mml-mir-20 |
| 225 | mne-miR-20 | 1194 | mne-mir-20 |
| 226 | ggo-miR-20 | 1195 | ggo-mir-20 |
| 227 | ppa-miR-20 | 1196 | ppa-mir-20 |
| 228 | ptr-miR-20 | 1197 | ptr-mir-20 |
| 229 | ppy-miR-20 | 1198 | ppy-mir-20 |
| 230 | lca-miR-20 | 1199 | lca-mir-20 |
| 231 | mdo-miR-20 | 1200 | mdo-mir-20 |
| 232 | gga-miR-20b | 1201 | gga-mir-20b |
| 233 | xtr-miR-20b | 1202 | xtr-mir-20b |
| 234 | bta-miR-20b | 1203 | bta-mir-20b |
| 235 | ssc-miR-20 | 1204 | ssc-mir-20 |
| 236 | dre-miR-20b | 1205 | dre-mir-20b |
| 237 | fru-miR-20 | 1206 | fru-mir-20 |
| 238 | tni-miR-20 | 1207 | tni-mir-20 |
| 239 | mmu-miR-106a | 1208 | mmu-mir-106a |
| 240 | age-miR-106a | 1209 | age-mir-106a |
| 241 | sla-miR-106a | 1210 | sla-mir-106a |
| 242 | mml-miR-106a | 1211 | mml-mir-106a |
| 243 | mne-miR-106a | 1212 | mne-mir-106a |
| 244 | ggo-miR-106a | 1213 | ggo-mir-106a |
| 245 | ppa-miR-106a | 1214 | ppa-mir-106a |
| 246 | ptr-miR-106a | 1215 | ptr-mir-106a |
| 247 | ppy-miR-106a | 1216 | ppy-mir-106a |
| 248 | gga-miR-106 | 1217 | gga-mir-106 |
| 249 | xtr-miR-106 | 1218 | xtr-mir-106 |
| 250 | bta-miR-106 | 1219 | bta-mir-106 |
| 251 | ssc-miR-106a | 1220 | ssc-mir-106a |
| 252 | mmu-miR-106b | 1221 | mmu-mir-106b |
| 253 | rno-miR-106b | 1222 | rno-mir-106b |
| 254 | age-miR-106b | 1223 | age-mir-106b |
| 255 | lla-miR-106b | 1224 | lla-mir-106b |
| 256 | sla-miR-106b | 1225 | sla-mir-106b |
| 257 | mml-miR-106b | 1226 | mml-mir-106b |
| 258 | mne-miR-106b | 1227 | mne-mir-106b |
| 259 | ggo-miR-106b | 1228 | ggo-mir-106b |
| 260 | ppa-miR-106b | 1229 | ppa-mir-106b |
| 261 | ptr-miR-106b | 1230 | ptr-mir-106b |
| 262 | ppy-miR-106b | 1231 | ppy-mir-106b |
| 263 | mmu-miR-24 | 1232 | mmu-mir-24-1 |
| | | 1233 | mmu-mir-24-2 |

Table 3-5

| | | | |
|---|---|---|---|
| 264 | rno-miR-24 | 1234 | rno-mir-24-1 |
| | | 1235 | rno-mir-24-2 |
| 265 | mml-miR-24 | 1236 | mml-mir-24-1 |
| | | 1237 | mml-mir-24-2 |
| 266 | mne-miR-24 | 1238 | mne-mir-24-1 |
| | | 1239 | mne-mir-24-2 |
| 267 | ggo-miR-24 | 1240 | ggo-mir-24 |
| 268 | ppa-miR-24 | 1241 | ppa-mir-24-1 |
| | | 1242 | ppa-mir-24-2 |
| 269 | ptr-miR-24 | 1243 | ptr-mir-24 |
| 270 | ppy-miR-24 | 1244 | ppy-mir-24-1 |
| | | 1245 | ppy-mir-24-2 |
| 271 | mdo-miR-24 | 1246 | mdo-mir-24-1 |
| | | 1247 | mdo-mir-24-2 |
| 272 | gga-miR-24 | 1248 | gga-mir-24 |
| 273 | xtr-miR-24b | 1249 | xtr-mir-24b |
| 274 | bta-miR-24 | 1250 | bta-mir-24 |
| 275 | ssc-miR-24 | 1251 | ssc-mir-24 |
| 276 | dre-miR-24 | 1252 | dre-mir-24-1 |
| | | 1253 | dre-mir-24-2 |
| | | 1254 | dre-mir-24-3 |
| | | 1255 | dre-mir-24-4 |
| 277 | fru-miR-24 | 1256 | fru-mir-24-1 |
| | | 1257 | fru-mir-24-2 |
| 278 | tni-miR-24 | 1258 | tni-mir-24-1 |
| | | 1259 | tni-mir-24-2 |
| 279 | mmu-miR-25 | 1260 | mmu-mir-25 |
| 280 | rno-miR-25 | 1261 | rno-mir-25 |
| 281 | lla-miR-25 | 1262 | lla-mir-25 |
| 282 | mml-miR-25 | 1263 | mml-mir-25 |
| 283 | mne-miR-25 | 1264 | mne-mir-25 |
| 284 | ggo-miR-25 | 1265 | ggo-mir-25 |
| 285 | ppa-miR-25 | 1266 | ppa-mir-25 |
| 286 | ptr-miR-25 | 1267 | ptr-mir-25 |
| 287 | ppy-miR-25 | 1268 | ppy-mir-25 |
| 288 | mdo-miR-25 | 1269 | mdo-mir-25 |
| 289 | xtr-miR-25 | 1270 | xtr-mir-25-1 |
| | | 1271 | xtr-mir-25-2 |
| 290 | bta-miR-25 | 1272 | bta-mir-25 |
| 291 | dre-miR-25 | 1273 | dre-mir-25 |
| 292 | fru-miR-25 | 1274 | fru-mir-25 |
| 293 | tni-miR-25 | 1275 | tni-mir-25 |
| 294 | mmu-miR-32 | 1276 | mmu-mir-32 |
| 295 | rno-miR-32 | 1277 | rno-mir-32 |
| 296 | sla-miR-32 | 1278 | sla-mir-32 |
| 297 | mml-miR-32 | 1279 | mml-mir-32 |
| 298 | mne-miR-32 | 1280 | mne-mir-32 |
| 299 | ggo-miR-32 | 1281 | ggo-mir-32 |
| 300 | ppa-miR-32 | 1282 | ppa-mir-32 |
| 301 | ptr-miR-32 | 1283 | ptr-mir-32 |
| 302 | ppy-miR-32 | 1284 | ppy-mir-32 |
| 303 | mdo-miR-32 | 1285 | mdo-mir-32 |
| 304 | gga-miR-32 | 1286 | gga-mir-32 |
| 305 | ssc-miR-32 | 1287 | ssc-mir-32 |
| 306 | mmu-miR-26a | 1288 | mmu-mir-26a-1 |
| | | 1289 | mmu-mir-26a-2 |
| 307 | rno-miR-26a | 1290 | rno-mir-26a |
| 308 | lla-miR-26a | 1291 | lla-mir-26a |
| 309 | mml-miR-26a | 1292 | mml-mir-26a |
| 310 | mne-miR-26a | 1293 | mne-mir-26a |
| 311 | ggo-miR-26a | 1294 | ggo-mir-26a |
| 312 | ppa-miR-26a | 1295 | ppa-mir-26a |
| 313 | ptr-miR-26a | 1296 | ptr-mir-26a |
| 314 | ppy-miR-26a | 1297 | ppy-mir-26a |
| 315 | gga-miR-26a | 1298 | gga-mir-26a |
| 316 | bta-miR-26a | 1299 | bta-mir-26a |
| 317 | ssc-miR-26a | 1300 | ssc-mir-26a |
| 318 | dre-miR-26a | 1301 | dre-mir-26a-1 |
| | | 1302 | dre-mir-26a-2 |
| | | 1303 | dre-mir-26a-3 |
| 319 | fru-miR-26 | 1304 | fru-mir-26 |
| 320 | tni-miR-26 | 1305 | tni-mir-26 |
| 321 | mmu-miR-26b | 1306 | mmu-mir-26b |
| 322 | rno-miR-26b | 1307 | rno-mir-26b |

Table 3-6

| | | | |
|---|---|---|---|
| 323 | bta-miR-26b | 1308 | bta-mir-26b |
| 324 | dre-miR-26b | 1309 | dre-mir-26b |
| 325 | mmu-miR-27b | 1310 | mmu-mir-27b |
| 326 | rno-miR-27a | 1311 | rno-mir-27a |
| 327 | rno-miR-27b | 1312 | rno-mir-27b |
| 328 | age-miR-27a | 1313 | age-mir-27a |
| 329 | sla-miR-27a | 1314 | sla-mir-27a |
| 330 | mml-miR-27a | 1315 | mml-mir-27a |
| 331 | mne-miR-27a | 1316 | mne-mir-27a |
| 332 | ggo-miR-27a | 1317 | ggo-mir-27a |
| 333 | ppa-miR-27a | 1318 | ppa-mir-27a |
| 334 | ptr-miR-27a | 1319 | ptr-mir-27a |
| 335 | ppy-miR-27a | 1320 | ppy-mir-27a |
| 336 | lca-miR-27a | 1321 | lca-mir-27a |
| 337 | mdo-miR-27a | 1322 | mdo-mir-27a |
| 338 | mdo-miR-27b | 1323 | mdo-mir-27b |
| 339 | gga-miR-27b | 1324 | gga-mir-27b |
| 340 | xtr-miR-27a | 1325 | xtr-mir-27a |
| 341 | xtr-miR-27b | 1326 | xtr-mir-27b |
| 342 | xtr-miR-27c | 1327 | xtr-mir-27c-1 |
| | | 1328 | xtr-mir-27c-2 |
| 343 | bta-miR-27a | 1329 | bta-mir-27a |
| 344 | bta-miR-27b | 1330 | bta-mir-27b |
| 345 | ssc-miR-27a | 1331 | ssc-mir-27a |
| 346 | dre-miR-27a | 1332 | dre-mir-27a |
| 347 | dre-miR-27b | 1333 | dre-mir-27b |
| 348 | dre-miR-27c | 1334 | dre-mir-27c |
| 349 | dre-miR-27d | 1335 | dre-mir-27d |
| 350 | dre-miR-27e | 1336 | dre-mir-27e |
| 351 | fru-miR-27b | 1337 | fru-mir-27b |
| 352 | fru-miR-27c | 1338 | fru-mir-27c |
| 353 | fru-miR-27e | 1339 | fru-mir-27e |
| 354 | tri-miR-27b | 1340 | tri-mir-27b |
| 355 | tri-miR-27c | 1341 | tri-mir-27c |
| 356 | tri-miR-27e | 1342 | tri-mir-27e |
| 357 | mmu-miR-28 | 1343 | mmu-mir-28 |
| 358 | rno-miR-28 | 1344 | rno-mir-28 |
| 359 | age-miR-28 | 1345 | age-mir-28 |
| 360 | lla-miR-28 | 1346 | lla-mir-28 |
| 361 | sla-miR-28 | 1347 | sla-mir-28 |
| 362 | mml-miR-28 | 1348 | mml-mir-28 |
| 363 | mne-miR-28 | 1349 | mne-mir-28 |
| 364 | ggo-miR-28 | 1350 | ggo-mir-28 |
| 365 | ppa-miR-28 | 1351 | ppa-mir-28 |
| 366 | ptr-miR-28 | 1352 | ptr-mir-28 |
| 367 | ppy-miR-28 | 1353 | ppy-mir-28 |
| 368 | ssc-miR-28 | 1354 | ssc-mir-28 |
| 369 | mmu-miR-29a | 1355 | mmu-mir-29a |
| 370 | rno-miR-29a | 1356 | rno-mir-29a |
| 371 | rno-miR-29b | 1357 | rno-mir-29b-1 |
| | | 1358 | rno-mir-29b-2 |
| 372 | age-miR-29b | 1359 | age-mir-29b |
| 373 | lla-miR-29b | 1360 | lla-mir-29b |
| 374 | sla-miR-29b | 1361 | sla-mir-29b |
| 375 | mne-miR-29b | 1362 | mne-mir-29b |
| 376 | ggo-miR-29b | 1363 | ggo-mir-29b-1 |
| | | 1364 | ggo-mir-29b-2 |
| 377 | ppa-miR-29b | 1365 | ppa-mir-29b-1 |
| | | 1366 | ppa-mir-29b-2 |
| 378 | ptr-miR-29b | 1367 | ptr-mir-29b-1 |
| | | 1368 | ptr-mir-29b-2 |
| 379 | ppy-miR-29b | 1369 | ppy-mir-29b-1 |
| | | 1370 | ppy-mir-29b-2 |
| 380 | mdo-miR-29a | 1371 | mdo-mir-29a |
| 381 | mdo-miR-29b | 1372 | mdo-mir-29b |
| 382 | gga-miR-29a | 1373 | gga-mir-29a |
| 383 | gga-miR-29b | 1374 | gga-mir-29b-1 |
| | | 1375 | gga-mir-29b-2 |
| 384 | xtr-miR-29a | 1376 | xtr-mir-29a |
| 385 | xtr-miR-29b | 1377 | xtr-mir-29b |
| 386 | xtr-miR-29d | 1378 | xtr-mir-29d |
| 387 | bta-miR-29b | 1379 | bta-mir-29b |
| 388 | ssc-miR-29b | 1380 | ssc-mir-29b |
| 389 | dre-miR-29a | 1381 | dre-mir-29a-1 |
| | | 1382 | dre-mir-29a-2 |
| 390 | dre-miR-29b | 1383 | dre-mir-29b-1 |
| | | 1384 | dre-mir-29b-2 |
| | | 1385 | dre-mir-29b-3 |

Table 3-7

| | | | |
|---|---|---|---|
| 391 | fru-miR-29a | 1386 | fru-mir-29a-1 |
| | | 1387 | fru-mir-29a-2 |
| 392 | fru-miR-29b | 1388 | fru-mir-29b-1 |
| | | 1389 | fru-mir-29b-2 |
| 393 | tri-miR-29a | 1390 | tri-mir-29a-1 |
| | | 1391 | tri-mir-29a-2 |
| 394 | tri-miR-29b | 1392 | tri-mir-29b-1 |
| | | 1393 | tri-mir-29b-2 |
| 395 | mmu-miR-29c | 1394 | mmu-mir-29c |
| 396 | rno-miR-29c | 1395 | rno-mir-29c |
| 397 | gga-miR-29c | 1396 | gga-mir-29c |
| 398 | xtr-miR-29c | 1397 | xtr-mir-29c |
| 399 | bta-miR-29c | 1398 | bta-mir-29c |
| 400 | ssc-miR-29c | 1399 | ssc-mir-29c |
| 401 | mmu-miR-30b | 1400 | mmu-mir-30b |
| 402 | mmu-miR-30d | 1401 | mmu-mir-30d |
| 403 | mmu-miR-30e | 1402 | mmu-mir-30e |
| 404 | rno-miR-30a | 1403 | rno-mir-30a |
| 405 | rno-miR-30d | 1404 | rno-mir-30d |
| 406 | rno-miR-30e | 1405 | rno-mir-30e |
| 407 | age-miR-30b | 1406 | age-mir-30b |
| 408 | lla-miR-30b | 1407 | lla-mir-30b |
| 409 | mml-miR-30a-5p | 1408 | mml-mir-30a |
| 410 | mml-miR-30b | 1409 | mml-mir-30b |
| 411 | mne-miR-30b | 1410 | mne-mir-30b |
| 412 | mne-miR-30d | 1411 | mne-mir-30d |
| 413 | ggo-miR-30a-5p | 1412 | ggo-mir-30a |
| 414 | ggo-miR-30b | 1413 | ggo-mir-30b |
| 415 | ggo-miR-30d | 1414 | ggo-mir-30d |
| 416 | ppa-miR-30a-5p | 1415 | ppa-mir-30a |
| 417 | ppa-miR-30b | 1416 | ppa-mir-30b |
| 418 | ppa-miR-30d | 1417 | ppa-mir-30d |
| 419 | ptr-miR-30a-5p | 1418 | ptr-mir-30a |
| 420 | ptr-miR-30b | 1419 | ptr-mir-30b |
| 421 | ptr-miR-30d | 1420 | ptr-mir-30d |
| 422 | ppy-miR-30a-5p | 1421 | ppy-mir-30a |
| 423 | mdo-miR-30a | 1422 | mdo-mir-30a |
| 424 | gga-miR-30a-5p | 1423 | gga-mir-30a |
| 425 | gga-miR-30b | 1424 | gga-mir-30b |
| 426 | gga-miR-30d | 1425 | gga-mir-30d |
| 427 | gga-miR-30e | 1426 | gga-mir-30e |
| 428 | xtr-miR-30a-5p | 1427 | xtr-mir-30a |
| 429 | xtr-miR-30b | 1428 | xtr-mir-30b |
| 430 | xtr-miR-30d | 1429 | xtr-mir-30d |
| 431 | xtr-miR-30e | 1430 | xtr-mir-30e |
| 432 | bta-miR-30a-5p | 1431 | bta-mir-30a |
| 433 | bta-miR-30b | 1432 | bta-mir-30b |
| 434 | bta-miR-30d | 1433 | bta-mir-30d |
| 435 | bta-miR-30e-5p | 1434 | bta-mir-30e |
| 436 | dre-miR-30a | 1435 | dre-mir-30a |
| 437 | dre-miR-30b | 1436 | dre-mir-30b |
| 438 | dre-miR-30d | 1437 | dre-mir-30d |
| 439 | dre-miR-30e | 1438 | dre-mir-30e-2 |
| 440 | fru-miR-30b | 1439 | fru-mir-30b |
| 441 | fru-miR-30d | 1440 | fru-mir-30d |
| 442 | tri-miR-30b | 1441 | tri-mir-30b |
| 443 | tri-miR-30d | 1442 | tri-mir-30d |
| 444 | mmu-miR-30c | 1443 | mmu-mir-30c-1 |
| | | 1444 | mmu-mir-30c-2 |
| 445 | rno-miR-30c | 1445 | rno-mir-30c-1 |
| | | 1446 | rno-mir-30c-2 |
| 446 | lla-miR-30c | 1447 | lla-mir-30c |
| 447 | mne-miR-30c | 1448 | mne-mir-30c |
| 448 | ptr-miR-30c | 1449 | ptr-mir-30c |
| 449 | gga-miR-30c | 1450 | gga-mir-30c-1 |
| | | 1451 | gga-mir-30c-2 |
| 450 | xtr-miR-30c | 1452 | xtr-mir-30c-1 |
| | | 1453 | xtr-mir-30c-2 |
| 451 | bta-miR-30c | 1454 | bta-mir-30c |
| 452 | ssc-miR-30c | 1455 | ssc-mir-30c |
| 453 | dre-miR-30c | 1456 | dre-mir-30c |
| 454 | fru-miR-30c | 1457 | fru-mir-30c |
| 455 | tri-miR-30c | 1458 | tri-mir-30c |
| 456 | age-miR-93 | 1459 | age-mir-93 |
| 457 | lla-miR-93 | 1460 | lla-mir-93 |
| 458 | sla-miR-93 | 1461 | sla-mir-93 |
| 459 | mml-miR-93 | 1462 | mml-mir-93 |
| 460 | mne-miR-93 | 1463 | mne-mir-93 |
| 461 | ggo-miR-93 | 1464 | ggo-mir-93 |
| 462 | ppa-miR-93 | 1465 | ppa-mir-93 |
| 463 | ptr-miR-93 | 1466 | ptr-mir-93 |
| 464 | ppy-miR-93 | 1467 | ppy-mir-93 |

Table 3-8

| | | | |
|---|---|---|---|
| 465 | mdo-miR-93 | 1468 | mdo-mir-93 |
| 466 | mmu-miR-302b | 1469 | mmu-mir-302b |
| 467 | mmu-miR-302d | 1470 | mmu-mir-302d |
| 468 | mdo-miR-302a | 1471 | mdo-mir-302a |
| 469 | mdo-miR-302b | 1472 | mdo-mir-302b |
| 470 | mdo-miR-302c | 1473 | mdo-mir-302c |
| 471 | mdo-miR-302d | 1474 | mdo-mir-302d |
| 472 | gga-miR-302b | 1475 | gga-mir-302b |
| 473 | gga-miR-302c | 1476 | gga-mir-302c |
| 474 | gga-miR-302d | 1477 | gga-mir-302d |
| 475 | xtr-miR-302 | 1478 | xtr-mir-302 |
| 476 | lla-miR-95 | 1479 | lla-mir-95 |
| 477 | sla-miR-95 | 1480 | sla-mir-95 |
| 478 | ggo-miR-95 | 1481 | ggo-mir-95 |
| 479 | ppa-miR-95 | 1482 | ppa-mir-95 |
| 480 | ptr-miR-95 | 1483 | ptr-mir-95 |
| 481 | ppy-miR-95 | 1484 | ppy-mir-95 |
| 482 | ssc-miR-95 | 1485 | ssc-mir-95 |
| 483 | mmu-miR-98 | 1486 | mmu-mir-98 |
| 484 | rno-miR-98 | 1487 | rno-mir-98 |
| 485 | age-miR-98 | 1488 | age-mir-98 |
| 486 | mml-miR-98 | 1489 | mml-mir-98 |
| 487 | ggo-miR-98 | 1490 | ggo-mir-98 |
| 488 | ppa-miR-98 | 1491 | ppa-mir-98 |
| 489 | ptr-miR-98 | 1492 | ptr-mir-98 |
| 490 | ppy-miR-98 | 1493 | ppy-mir-98 |
| 491 | xtr-miR-98 | 1494 | xtr-mir-98 |
| 492 | bta-miR-98 | 1495 | bta-mir-98 |
| 493 | mmu-miR-130a | 1496 | mmu-mir-130a |
| 494 | rno-miR-130a | 1497 | rno-mir-130a |
| 495 | mml-miR-130a | 1498 | mml-mir-130a |
| 496 | mne-miR-130a | 1499 | mne-mir-130a |
| 497 | ggo-miR-130a | 1500 | ggo-mir-130a |
| 498 | ppa-miR-130a | 1501 | ppa-mir-130a |
| 499 | mdo-miR-130a | 1502 | mdo-mir-130a |
| 500 | gga-miR-130a | 1503 | gga-mir-130a |
| 501 | xtr-miR-130a | 1504 | xtr-mir-130a |
| 502 | dre-miR-130a | 1505 | dre-mir-130a-1 |
| | | 1506 | dre-mir-130a-2 |
| 503 | dre-miR-130c | 1507 | dre-mir-130c-1 |
| | | 1508 | dre-mir-130c-2 |
| 504 | fru-miR-130 | 1509 | fru-mir-130 |
| 505 | tri-miR-130 | 1510 | tri-mir-130 |
| 506 | mmu-miR-130b | 1511 | mmu-mir-130b |
| 507 | rno-miR-130b | 1512 | rno-mir-130b |
| 508 | gga-miR-130b | 1513 | gga-mir-130b |
| 509 | xtr-miR-130b | 1514 | xtr-mir-130b |
| 510 | dre-miR-130b | 1515 | dre-mir-130b |
| 511 | mmu-miR-301a | 1516 | mmu-mir-301a |
| 512 | mmu-miR-301b | 1517 | mmu-mir-301b |
| 513 | rno-miR-301a | 1518 | rno-mir-301a |
| 514 | rno-miR-301b | 1519 | rno-mir-301b |
| 515 | gga-miR-301 | 1520 | gga-mir-301 |
| 516 | xtr-miR-301 | 1521 | xtr-mir-301-1 |
| | | 1522 | xtr-mir-301-2 |
| 517 | dre-miR-301a | 1523 | dre-mir-301a-1 |
| | | 1524 | dre-mir-301a-2 |
| 518 | dre-miR-301b | 1525 | dre-mir-301b |
| 519 | dre-miR-301c | 1526 | dre-mir-301c |
| 520 | fru-miR-301 | 1527 | fru-mir-301 |
| 521 | tri-miR-301 | 1528 | tri-mir-301 |
| 522 | mmu-miR-132 | 1529 | mmu-mir-132 |
| 523 | rno-miR-132 | 1530 | rno-mir-132 |
| 524 | mdo-miR-132 | 1531 | mde-mir-132 |
| 525 | xtr-miR-132 | 1532 | xtr-mir-132 |
| 526 | bta-miR-132 | 1533 | bta-mir-132 |
| 527 | dre-miR-132 | 1534 | dre-mir-132-1 |
| | | 1535 | dre-mir-132-2 |
| 528 | fru-miR-132 | 1536 | fru-mir-132 |
| 529 | tri-miR-132 | 1537 | tri-mir-132 |
| 530 | mmu-miR-212 | 1538 | mmu-mir-212 |
| 531 | rno-miR-212 | 1539 | rno-mir-212 |
| 532 | mdo-miR-212 | 1540 | mdo-mir-212 |
| 533 | xtr-miR-212 | 1541 | xtr-mir-212 |
| 534 | dre-miR-212 | 1542 | dre-mir-212 |
| 535 | fru-miR-212 | 1543 | fru-mir-212 |
| 536 | tri-miR-212 | 1544 | tri-mir-212 |
| 537 | age-miR-133a | 1545 | age-mir-133a |
| 538 | lla-miR-133a | 1546 | lla-mir-133a |
| 539 | sla-miR-133a | 1547 | sla-mir-133a |
| 540 | mne-miR-133a | 1548 | mne-mir-133a |
| 541 | ggo-miR-133a | 1549 | ggo-mir-133a |

Table 3-9

| | | | |
|---|---|---|---|
| 542 | ppa-miR-133a | 1550 | ppa-mir-133a |
| 543 | ptr-miR-133a | 1551 | ptr-mir-133a |
| 544 | mdo-miR-133a | 1552 | mdo-mir-133a |
| 545 | gga-miR-133a | 1553 | gga-mir-133a-1 |
| | | 1554 | gga-mir-133a-2 |
| 546 | gga-miR-133c | 1555 | gga-mir-133c |
| 547 | xla-miR-133a | 1556 | xla-mir-133a |
| 548 | xtr-miR-133a | 1557 | xtr-mir-133a |
| 549 | xtr-miR-133d | 1558 | xtr-mir-133d |
| 550 | gga-miR-133b | 1559 | gga-mir-133b |
| 551 | xtr-miR-133b | 1560 | xtr-mir-133b |
| 552 | mmu-miR-134 | 1561 | mmu-mir-134 |
| 553 | rno-miR-134 | 1562 | rno-mir-134 |
| 554 | mne-miR-134 | 1563 | mne-mir-134 |
| 555 | ggo-miR-134 | 1564 | ggo-mir-134 |
| 556 | ppa-miR-134 | 1565 | ppa-mir-134 |
| 557 | ppy-miR-134 | 1566 | ppy-mir-134 |
| 558 | mdo-miR-137 | 1567 | mdo-mir-137 |
| 559 | gga-miR-137 | 1568 | gga-mir-137 |
| 560 | xtr-miR-137 | 1569 | xtr-mir-137-1 |
| | | 1570 | xtr-mir-137-2 |
| | | 1571 | xtr-mir-137-3 |
| 561 | mmu-miR-142-3p | 1572 | mmu-mir-142 |
| 562 | rno-miR-142-3p | 1573 | rno-mir-142 |
| 563 | gga-miR-142-3p | 1574 | gga-mir-142 |
| 564 | xtr-miR-142-3p | 1575 | xtr-mir-142-1 |
| | | 1576 | xtr-mir-142-2 |
| 565 | mmu-miR-146a | 1577 | mmu-mir-146a |
| 566 | rno-miR-146a | 1578 | rno-mir-146a |
| 567 | gga-miR-146a | 1579 | gga-mir-146a |
| 568 | gga-miR-146b | 1580 | gga-mir-146b |
| 569 | xtr-miR-146 | 1581 | xtr-mir-146 |
| 570 | dre-miR-146a | 1582 | dre-mir-146a |
| 571 | dre-miR-146b | 1583 | dre-mir-146b-1 |
| | | 1584 | dre-mir-146b-2 |
| 572 | sla-miR-147 | 1585 | sla-mir-147 |
| 573 | mne-miR-147 | 1586 | mne-mir-147 |
| 574 | ppa-miR-147 | 1587 | ppa-mir-147 |
| 575 | ptr-miR-147 | 1588 | ptr-mir-147 |
| 576 | ppy-miR-147 | 1589 | ppy-mir-147 |
| 577 | mmu-miR-149 | 1590 | mmu-mir-149 |
| 578 | mmu-miR-181c | 1591 | mmu-mir-181c |
| 579 | rno-miR-181c | 1592 | rno-mir-181c |
| 580 | mml-miR-181c | 1593 | mml-mir-181c |
| 581 | ggo-miR-181c | 1594 | ggo-mir-181c |
| 582 | ppa-miR-181c | 1595 | ppa-mir-181c |
| 583 | ptr-miR-181c | 1596 | ptr-mir-181c |
| 584 | mdo-miR-181c | 1597 | mdo-mir-181c |
| 585 | bta-miR-181c | 1598 | bta-mir-181c |
| 586 | ssc-miR-181c | 1599 | ssc-mir-181c |
| 587 | dre-miR-181c | 1600 | dre-mir-181c |
| 588 | mmu-miR-181d | 1601 | mmu-mir-181d |
| 589 | rno-miR-181d | 1602 | rno-mir-181d |
| 590 | xtr-miR-182* | 1603 | xtr-mir-182 |
| 591 | dre-miR-182* | 1604 | dre-mir-182 |
| 592 | mmu-miR-183 | 1605 | mmu-mir-183 |
| 593 | rno-miR-183 | 1606 | rno-mir-133 |
| 594 | sla-miR-183 | 1607 | sla-mir-183 |
| 595 | mml-miR-183 | 1608 | mml-mir-183 |
| 596 | mne-miR-183 | 1609 | mne-mir-183 |
| 597 | ggo-miR-183 | 1610 | ggo-mir-183 |
| 598 | ppa-miR-183 | 1611 | ppa-mir-183 |
| 599 | ptr-miR-183 | 1612 | ptr-mir-183 |
| 600 | mdo-miR-183 | 1613 | mdo-mir-183 |
| 601 | gga-miR-183 | 1614 | gga-mir-183 |
| 602 | xtr-miR-183 | 1615 | xtr-mir-183 |
| 603 | ssc-miR-183 | 1616 | ssc-mir-183 |
| 604 | dre-miR-183 | 1617 | dre-mir-183 |
| 605 | fru-miR-183 | 1618 | fru-mir-183 |
| 606 | tri-miR-183 | 1619 | tri-mir-183 |
| 607 | mmu-miR-184 | 1620 | mmu-mir-184 |
| 608 | rno-miR-184 | 1621 | rno-mir-184 |
| 609 | mne-miR-184 | 1622 | mne-mir-184 |
| 610 | ptr-miR-184 | 1623 | ptr-mir-184 |
| 611 | ppy-miR-184 | 1624 | ppy-mir-184 |
| 612 | mdo-miR-184 | 1625 | mdo-mir-184 |
| 613 | gga-miR-184 | 1626 | gga-mir-184 |
| 614 | xtr-miR-184 | 1627 | xtr-mir-184 |
| 615 | ssc-miR-184 | 1628 | ssc-mir-184 |
| 616 | dre-miR-184 | 1629 | dre-mir-184-2 |
| | | 1630 | dre-mir-184 |
| 617 | fru-miR-184 | 1631 | fru-mir-184 |

Table 3-10

| | | | |
|---|---|---|---|
| 618 | tri-miR-184 | 1632 | tri-mir-184 |
| 619 | mmu-miR-192 | 1633 | mmu-mir-192 |
| 620 | rno-miR-192 | 1634 | rno-mir-192 |
| 621 | xtr-miR-192 | 1635 | xtr-mir-192 |
| 622 | bta-miR-192 | 1636 | bta-mir-192 |
| 623 | dre-miR-192 | 1637 | dre-mir-192 |
| 624 | fru-miR-192 | 1638 | fru-mir-192 |
| 625 | tri-miR-192 | 1639 | tri-mir-192 |
| 626 | mmu-miR-215 | 1640 | mmu-mir-215 |
| 627 | rno-miR-215 | 1641 | rno-mir-215 |
| 628 | mml-miR-215 | 1642 | mml-mir-215 |
| 629 | mne-miR-215 | 1643 | mne-mir-215 |
| 630 | ggo-miR-215 | 1644 | ggo-mir-215 |
| 631 | ptr-miR-215 | 1645 | ptr-mir-215 |
| 632 | ppy-miR-215 | 1646 | ppy-mir-215 |
| 633 | gga-miR-215 | 1647 | gga-mir-215 |
| 634 | xtr-miR-215 | 1648 | xtr-mir-215 |
| 635 | bta-miR-215 | 1649 | bta-mir-215 |
| 636 | rno-miR-193 | 1650 | rno-mir-193 |
| 637 | mdo-miR-193 | 1651 | mdo-mir-193 |
| 638 | gga-miR-193 | 1652 | gga-mir-193 |
| 639 | xtr-miR-193 | 1653 | xtr-mir-193 |
| 640 | bta-miR-193a | 1654 | bta-mir-193a |
| 641 | dre-miR-193a | 1655 | dre-mir-193a-1 |
| | | 1656 | dre-mir-193a-2 |
| | | 1657 | dre-mir-193a-3 |
| 642 | fru-miR-193 | 1658 | fru-mir-193 |
| 643 | tri-miR-193 | 1659 | tri-mir-193 |
| 644 | mmu-miR-193b | 1660 | mmu-mir-193b |
| 645 | dre-miR-193b | 1661 | dre-mir-193b |
| 646 | mmu-miR-195 | 1662 | mmu-mir-195 |
| 647 | rno-miR-195 | 1663 | rno-mir-195 |
| 648 | ggo-miR-195 | 1664 | ggo-mir-195 |
| 649 | ppa-miR-195 | 1665 | ppa-mir-195 |
| 650 | bta-miR-195 | 1666 | bta-mir-195 |
| 651 | mmu-miR-497 | 1667 | mmu-mir-497 |
| 652 | rno-miR-497 | 1668 | rno-mir-497 |
| 653 | bta-miR-497 | 1669 | bta-mir-497 |
| 654 | mmu-miR-196a | 1670 | mmu-mir-196a-1 |
| | | 1671 | mmu-mir-196a-2 |
| 655 | rno-miR-196a | 1672 | rno-mir-196a |
| 656 | age-miR-196 | 1673 | age-mir-196 |
| 657 | lla-miR-196 | 1674 | lla-mir-196 |
| 658 | mml-miR-196 | 1675 | mml-mir-196 |
| 659 | ggo-miR-196 | 1676 | ggo-mir-196-1 |
| | | 1677 | ggo-mir-196-2 |
| 660 | ppa-miR-196 | 1678 | ppa-mir-196 |
| 661 | ptr-miR-196 | 1679 | ptr-mir-196 |
| 662 | ppy-miR-196 | 1680 | ppy-mir-196-1 |
| | | 1681 | ppy-mir-196-2 |
| 663 | gga-miR-196 | 1682 | gga-mir-196-1 |
| | | 1683 | gga-mir-196-2 |
| | | 1684 | gga-mir-196-3 |
| 664 | xtr-miR-196a | 1685 | xtr-mir-196a |
| 665 | ssc-miR-196 | 1686 | ssc-mir-196 |
| 666 | dre-miR-196a | 1687 | dre-mir-196a-1 |
| | | 1688 | dre-mir-196a-2 |
| 667 | fru-miR-196a | 1689 | fru-mir-196a-1 |
| | | 1690 | fru-mir-196a-2 |
| 668 | tri-miR-196a | 1691 | tri-mir-196a-1 |
| | | 1692 | tri-mir-196a-2 |
| 669 | mmu-miR-196b | 1693 | mmu-mir-196b |
| 670 | rno-miR-196b | 1694 | rno-mir-196b |
| 671 | mdo-miR-196b | 1695 | mdo-mir-196b |
| 672 | xtr-miR-196b | 1696 | xtr-mir-196b |
| 673 | dre-miR-196b | 1697 | dre-mir-196b |
| 674 | fru-miR-196b | 1698 | fru-mir-196b |
| 675 | tri-miR-196b | 1699 | tri-mir-196b |
| 676 | mmu-miR-197 | 1700 | mmu-mir-197 |
| 677 | age-miR-197 | 1701 | age-mir-197 |
| 678 | mne-miR-197 | 1702 | mne-mir-197 |
| 679 | ppa-miR-197 | 1703 | ppa-mir-197 |
| 680 | ptr-miR-197 | 1704 | ptr-mir-197 |
| 681 | ppy-miR-197 | 1705 | ppy-mir-197 |
| 682 | xtr-miR-199a* | 1706 | xtr-mir-199a |
| 683 | dre-miR-199* | 1707 | dre-mir-199-1 |
| | | 1708 | dre-mir-199-2 |
| | | 1709 | dre-mir-199-3 |
| 684 | mmu-miR-200a | 1710 | mmu-mir-200a |
| 685 | rno-miR-200a | 1711 | rno-mir-200a |
| 686 | mdo-miR-200a | 1712 | mdo-mir-200a |
| 687 | gga-miR-200a | 1713 | gga-mir-200a |

Table 3-11

| | | | |
|---|---|---|---|
| 688 | xtr-miR-200a | 1714 | xtr-mir-200a |
| 689 | bta-miR-200a | 1715 | bta-mir-200a |
| 690 | dre-miR-200a | 1716 | dre-mir-200a |
| 691 | fru-miR-200a | 1717 | fru-mir-200a |
| 692 | tni-miR-200a | 1718 | tni-mir-200a |
| 693 | mmu-miR-204 | 1719 | mmu-mir-204 |
| 694 | rno-miR-204 | 1720 | rno-mir-204 |
| 695 | sta-miR-204 | 1721 | sta-mir-204 |
| 696 | mne-miR-204 | 1722 | mne-mir-204 |
| 697 | ggo-miR-204 | 1723 | ggo-mir-204 |
| 698 | ppa-miR-204 | 1724 | ppa-mir-204 |
| 699 | ptr-miR-204 | 1725 | ptr-mir-204 |
| 700 | ppy-miR-204 | 1726 | ppy-mir-204 |
| 701 | mdo-miR-204 | 1727 | mdo-mir-204 |
| 702 | gga-miR-204 | 1728 | gga-mir-204-1 |
| | | 1729 | gga-mir-204-2 |
| 703 | xtr-miR-204 | 1730 | xtr-mir-204-1 |
| | | 1731 | xtr-mir-204-2 |
| 704 | bta-miR-204 | 1732 | bta-mir-204 |
| 705 | ssc-miR-204 | 1733 | ssc-mir-204 |
| 706 | dre-miR-204 | 1734 | dre-mir-204-1 |
| | | 1735 | dre-mir-204-2 |
| | | 1736 | dre-mir-204-3 |
| 707 | fru-miR-204 | 1737 | fru-mir-204 |
| 708 | tni-miR-204a | 1738 | tni-mir-204a |
| 709 | mmu-miR-211 | 1739 | mmu-mir-211 |
| 710 | rno-miR-211 | 1740 | rno-mir-211 |
| 711 | mml-miR-211 | 1741 | mml-mir-211 |
| 712 | mne-miR-211 | 1742 | mne-mir-211 |
| 713 | ppy-miR-211 | 1743 | ppy-mir-211 |
| 714 | gga-miR-211 | 1744 | gga-mir-211 |
| 715 | mmu-miR-205 | 1745 | mmu-mir-205 |
| 716 | rno-miR-205 | 1746 | rno-mir-205 |
| 717 | age-miR-205 | 1747 | age-mir-205 |
| 718 | lla-miR-205 | 1748 | lle-mir-205 |
| 719 | mne-miR-205 | 1749 | mne-mir-205 |
| 720 | ggo-miR-205 | 1750 | ggo-mir-205 |
| 721 | ppa-miR-205 | 1751 | ppa-mir-205 |
| 722 | ptr-miR-205 | 1752 | ptr-mir-205 |
| 723 | gga-miR-205a | 1753 | gga-mir-205a |
| 724 | gga-miR-205b | 1754 | gga-mir-205b |
| 725 | xtr-miR-205a | 1755 | xtr-mir-205a |
| 726 | bta-miR-205 | 1756 | bta-mir-205 |
| 727 | ssc-miR-205 | 1757 | ssc-mir-205 |
| 728 | dre-miR-205 | 1758 | dre-mir-205 |
| 729 | fru-miR-205 | 1759 | fru-mir-205 |
| 730 | tni-miR-205 | 1760 | tni-mir-205 |
| 731 | mmu-miR-208 | 1761 | mmu-mir-208 |
| 732 | rno-miR-208 | 1762 | rno-mir-208 |
| 733 | mdo-miR-208 | 1763 | mdo-mir-208 |
| 734 | xtr-miR-208 | 1764 | xtr-mir-208 |
| 735 | mmu-miR-222 | 1765 | mmu-mir-222 |
| 736 | rno-miR-222 | 1766 | rno-mir-222 |
| 737 | age-miR-222 | 1767 | age-mir-222 |
| 738 | gga-miR-222 | 1768 | gga-mir-222 |
| 739 | xtr-miR-222 | 1769 | xtr-mir-222 |
| 740 | bta-miR-222 | 1770 | bta-mir-222 |
| 741 | dre-miR-222 | 1771 | dre-mir-222 |
| 742 | fru-miR-222 | 1772 | fru-mir-222 |
| 743 | tni-miR-222 | 1773 | tni-mir-222 |
| 744 | mmu-miR-302b* | 1469 | mmu-mir-302b |
| 745 | gga-miR-302b* | 1475 | gga-mir-302b |
| 746 | mmu-miR-324-3p | 1774 | mmu-mir-324 |
| 747 | rno-miR-324-3p | 1775 | rno-mir-324 |
| 748 | mmu-miR-324-5p | 1774 | mmu-mir-324 |
| 749 | rno-miR-324-5p | 1775 | rno-mir-324 |
| 750 | mmu-miR-329 | 1776 | mmu-mir-329 |
| 751 | rno-miR-329 | 1777 | rno-mir-329 |
| 752 | rno-miR-331 | 1778 | rno-mir-331 |
| 753 | bta-miR-331 | 1779 | bta-mir-331 |
| 754 | bta-miR-342 | 1780 | bta-mir-342 |
| 755 | mmu-miR-346 | 1781 | mmu-mir-346 |
| 756 | rno-miR-346 | 1782 | rno-mir-346 |
| 757 | mmu-miR-376b | 1783 | mm-mir-376b |
| 758 | mmu-miR-377 | 1784 | mmu-mir-377 |
| 759 | rno-miR-377 | 1785 | rno-mir-377 |
| 760 | bta-miR-380-5p | 1786 | bta-mir-380 |
| 761 | mmu-miR-382 | 1787 | mmu-mir-382 |
| 762 | rno-miR-382 | 1788 | rno-mir-382 |
| 763 | rno-miR-412 | 1789 | rno-mir-412 |
| 764 | rno-miR-423 | 1790 | rno-mir-423 |
| 765 | mmu-miR-431 | 1791 | mmu-mir-431 |

Table 3-12

| | | | |
|---|---|---|---|
| 766 | rno-miR-431 | 1792 | rno-mir-431 |
| 767 | oar-miR-431 | 1793 | oar-mir-431 |
| 768 | mmu-miR-433 | 1794 | mmu-mir-433 |
| 769 | rno-miR-433 | 1795 | rno-mir-433 |
| 770 | mmu-miR-449b | 1796 | mmu-mir-449b |
| 771 | mmu-miR-449c | 1797 | mmu-mir-449c |
| 772 | rno-miR-449a | 1798 | rno-mir-449a |
| 773 | cfa-miR-449 | 1799 | cfa-mir-449 |
| 774 | mdo-miR-449 | 1800 | mdo-mir-449 |
| 775 | gga-miR-449 | 1801 | gga-mir-449 |
| 776 | xtr-miR-449 | 1802 | xtr-mir-449 |
| 777 | mmu-miR-451 | 1803 | mmu-mir-451 |
| 778 | rno-miR-451 | 1804 | rno-mir-451 |
| 779 | mdo-miR-451 | 1805 | mdo-mir-451 |
| 780 | gga-miR-451 | 1806 | gga-mir-451 |
| 781 | xtr-miR-451 | 1807 | xtr-mir-451 |
| 782 | dre-miR-451 | 1808 | dre-mir-451 |
| 783 | mmu-miR-452 | 1809 | mmu-mir-452 |
| 784 | rno-miR-455 | 1810 | rno-mir-455 |
| 785 | gga-miR-455 | 1811 | gga-mir-455 |
| 786 | xtr-miR-455 | 1812 | xtr-mir-455 |
| 787 | bta-miR-455 | 1813 | bta-mir-455 |
| 788 | dre-miR-455 | 1814 | dre-mir-455 |
| 789 | fru-miR-455 | 1815 | fru-mir-455 |
| 790 | tri-miR-455 | 1816 | tri-mir-455 |
| 791 | rno-miR-483 | 1817 | rno-mir-483 |
| 792 | gga-miR-489 | 1818 | gga-mir-489 |
| 793 | xtr-miR-489 | 1819 | xtr-mir-489 |
| 794 | dre-miR-489 | 1820 | dre-mir-489 |
| 795 | fru-miR-489 | 1821 | fru-mir-489 |
| 796 | tni-miR-489 | 1822 | tri-mir-489 |
| 797 | mmu-miR-491 | 1823 | mmu-mir-491 |
| 798 | mmu-miR-494 | 1824 | mmu-mir-494 |
| 799 | rno-miR-494 | 1825 | rno-mir-494 |
| 800 | rno-miR-501 | 1826 | rno-mir-501 |
| 801 | mmu-miR-504 | 1827 | mmu-mir-504 |
| 802 | rno-miR-505 | 1828 | rno-mir-505 |
| 803 | hsa-miR-613 | 1829 | hsa-mir-613 |
| 804 | hsa-miR-10b | 1830 | hsa-mir-10b |
| 805 | hsa-miR-17 | 1831 | hsa-mir-17 |
| 806 | hsa-miR-519d | 1832 | hsa-mir-519d |
| 807 | hsa-miR-526b* | 1833 | hsa-mir-526b |
| 808 | mmu-miR-17 | 1834 | mmu-mir-17 |
| 809 | mmu-miR-93 | 1835 | mmu-mir-93 |
| 810 | rno-miR-17 | 1836 | rno-mir-17-1 |
| | | 1837 | rno-mir-17-2 |
| 811 | rno-miR-93 | 1838 | rno-mir-93 |
| 812 | xtr-miR-93a | 1839 | xtr-mir-93a |
| 813 | bta-miR-93 | 1840 | bta-mir-93 |
| 814 | dre-miR-17a | 1841 | dre-mir-17a-1 |
| | | 1842 | dre-mir-17a-2 |
| 815 | dre-miR-93 | 1843 | dre-mir-93 |
| 816 | fru-miR-17 | 1844 | fru-mir-17-1 |
| | | 1845 | fru-mir-17-2 |
| 817 | tri-miR-17 | 1846 | tri-mir-17-1 |
| | | 1847 | tri-mir-17-2 |
| 818 | hsa-miR-92a | 1848 | hsa-mir-92a-1 |
| | | 1849 | hsa-mir-92a-2 |
| 819 | hsa-miR-92b | 1850 | hsa-mir-92b |
| 820 | hsa-miR-363 | 1851 | hsa-mir-363 |
| 821 | hsa-miR-367 | 1852 | hsa-mir-367 |
| 822 | mmu-miR-92a | 1853 | hsa-mir-92a-1 |
| | | 1854 | mmu-mir-92a-2 |
| 823 | mmu-miR-92b | 1855 | mmu-mir-92b |
| 824 | mmu-miR-363 | 1856 | mmu-mir-363 |
| 825 | mmu-miR-367 | 1857 | mmu-mir-367 |
| 826 | rno-miR-92a | 1858 | rno-mir-92a-1 |
| | | 1859 | rno-mir-92a-2 |
| 827 | rno-miR-92b | 1860 | rno-mir-92b |
| 828 | rno-miR-363 | 1861 | rno-mir-363 |
| 829 | age-miR-92 | 1862 | age-mir-92-1 |
| | | 1863 | age-mir-92-2 |
| 830 | lla-miR-92 | 1864 | lla-mir-92-1 |
| | | 1865 | lla-mir-92-2 |
| 831 | sla-miR-92 | 1866 | sla-mir-92-1 |
| | | 1867 | sla-mir-92-2 |
| 832 | mml-miR-92 | 1868 | mml-mir-92 |
| 833 | mne-miR-92 | 1869 | mne-mir-92 |
| 834 | ggo-miR-92 | 1870 | ggo-mir-92-1 |
| | | 1871 | ggo-mir-92-2 |
| 835 | ppa-miR-92 | 1872 | ppa-mir-92-1 |
| | | 1873 | ppa-mir-92-2 |

Table 3-13

| | | | |
|---|---|---|---|
| 836 | ptr-miR-92 | 1874 | ptr-mir-92-1 |
| | | 1875 | ptr-mir-92-2 |
| 837 | ppy-miR-92 | 1876 | ppy-mir-92-1 |
| | | 1877 | ppy-mir-92-2 |
| 838 | lca-miR-92 | 1878 | lca-mir-92-1 |
| | | 1879 | lca-mir-92-2 |
| 839 | mdo-miR-92 | 1880 | mdo-mir-92 |
| 840 | gga-miR-92 | 1881 | gga-mir-92 |
| 841 | gga-miR-367 | 1882 | gga-mir-367 |
| 842 | xtr-miR-92a | 1883 | xtr-mir-92a-1 |
| | | 1884 | xtr-mir-92a-2 |
| 843 | xtr-miR-92b | 1885 | xtr-mir-92b |
| 844 | xtr-miR-363-3p | 1886 | xtr-mir-363 |
| 845 | xtr-miR-367 | 1887 | xtr-mir-367 |
| 846 | bta-miR-92 | 1888 | bta-mir-92 |
| 847 | dre-miR-92a | 1889 | dre-mir-92a-1 |
| | | 1890 | dre-mir-92a-2 |
| 848 | dre-miR-92b | 1891 | dre-mir-92b |
| 849 | dre-miR-363 | 1892 | dre-mir-363 |
| 850 | fru-miR-92 | 1893 | fru-mir-92-1 |
| | | 1894 | fru-mir-92-2 |
| 851 | tni-miR-92 | 1895 | tni-mir-92-1 |
| | | 1896 | tni-mir-92-2 |
| 852 | hsa-miR-27a | 1897 | hsa-mir-27a |
| 853 | hsa-miR-708 | 1898 | hsa-mir-708 |
| 854 | mmu-miR-708 | 1899 | mmu-mir-708 |
| 855 | rno-miR-708 | 1900 | rno-mir-708 |
| 856 | hsa-miR-29b | 1901 | hsa-mir-29b-1 |
| | | 1902 | hsa-mir-29b-2 |
| 857 | hsa-miR-30a | 1903 | hsa-mir-30a |
| 858 | hsa-miR-30d | 1904 | hsa-mir-30d |
| 859 | hsa-miR-30e | 1905 | hsa-mir-30e |
| 860 | mmu-miR-384-5p | 1906 | mmu-mir-384 |
| 861 | rno-miR-384-5p | 1907 | rno-mir-384 |
| 862 | hsa-miR-302a | 1010 | hsa-mir-302a |
| 863 | hsa-miR-302b | 1011 | hsa-mir-302b |
| 864 | hsa-miR-302c | 1908 | hsa-mir-302c |
| 865 | hsa-miR-520a-3p | 1909 | hsa-mir-520a |
| 866 | hsa-miR-520b | 1910 | hsa-mir-520b |
| 867 | hsa-miR-520c-3p | 1911 | hsa-mir-520c |
| 868 | hsa-miR-520d-3p | 1912 | hsa-mir-520d |
| 869 | hsa-miR-520e | 1913 | hsa-mir-520e |
| 870 | mmu-miR-291a-3p | 1914 | mmu-mir-291a |
| 871 | mmu-miR-294 | 1915 | mmu-mir-294 |
| 872 | mmu-miR-295 | 1916 | mmu-mir-295 |
| 873 | rno-miR-291a-3p | 1917 | rno-mir-291a |
| 874 | xla-miR-427 | 1918 | xla-mir-427 |
| 875 | xla-miR-428 | 1919 | xla-mir-428 |
| 876 | xtr-miR-428 | 1920 | xtr-mir-428 |

Table 3-14

| | | | |
|---|---|---|---|
| 877 | dre-miR-430a | 1921 | dre-mir-430a-1 |
| | | 1922 | dre-mir-430a-2 |
| | | 1923 | dre-mir-430a-3 |
| | | 1924 | dre-mir-430a-4 |
| | | 1925 | dre-mir-430a-5 |
| | | 1926 | dre-mir-430a-6 |
| | | 1927 | dre-mir-430a-7 |
| | | 1928 | dre-mir-430a-8 |
| | | 1929 | dre-mir-430a-9 |
| | | 1930 | dre-mir-430a-10 |
| | | 1931 | dre-mir-430a-11 |
| | | 1932 | dre-mir-430a-12 |
| | | 1933 | dre-mir-430a-13 |
| | | 1934 | dre-mir-430a-14 |
| | | 1935 | dre-mir-430a-15 |
| | | 1936 | dre-mir-430a-16 |
| | | 1937 | dre-mir-430a-17 |
| | | 1938 | dre-mir-430a-18 |
| | | 1939 | dre-mir-430a-19 |
| | | 1940 | dre-mir-430a-20 |
| | | 1941 | dre-mir-430a-21 |
| | | 1942 | dre-mir-430a-22 |
| | | 1943 | dre-mir-430a-23 |
| 878 | dre-miR-430b | 1944 | dre-mir-430b-1 |
| | | 1945 | dre-mir-430b-2 |
| | | 1946 | dre-mir-430b-3 |
| | | 1947 | dre-mir-430b-4 |
| | | 1948 | dre-mir-430b-5 |
| | | 1949 | dre-mir-430b-6 |
| | | 1950 | dre-mir-430b-7 |
| | | 1951 | dre-mir-430b-8 |
| | | 1952 | dre-mir-430b-9 |
| | | 1953 | dre-mir-430b-10 |
| | | 1954 | dre-mir-430b-11 |
| | | 1955 | dre-mir-430b-12 |
| | | 1956 | dre-mir-430b-13 |
| | | 1957 | dre-mir-430b-14 |
| | | 1958 | dre-mir-430b-15 |
| | | 1959 | dre-mir-430b-16 |
| | | 1960 | dre-mir-430b-17 |
| | | 1961 | dre-mir-430b-18 |
| | | 1962 | dre-mir-430b-19 |
| | | 1963 | dre-mir-430b-20 |
| | | 1964 | dre-mir-430b-21 |
| | | 1965 | dre-mir-430b-22 |
| | | 1966 | dre-mir-430b-23 |
| 879 | dre-miR-430c | 1967 | dre-mir-430c-1 |
| | | 1968 | dre-mir-430c-2 |
| | | 1969 | dre-mir-430c-3 |
| | | 1970 | dre-mir-430c-4 |
| | | 1971 | dre-mir-430c-5 |
| | | 1972 | dre-mir-430c-6 |
| | | 1973 | dre-mir-430c-7 |
| | | 1974 | dre-mir-430c-8 |
| | | 1975 | dre-mir-430c-9 |
| | | 1976 | dre-mir-430c-10 |
| | | 1977 | dre-mir-430c-11 |
| | | 1978 | dre-mir-430c-12 |
| | | 1979 | dre-mir-430c-13 |
| | | 1980 | dre-mir-430c-14 |
| | | 1981 | dre-mir-430c-15 |
| | | 1982 | dre-mir-430c-16 |
| | | 1983 | dre-mir-430c-17 |
| | | 1984 | dre-mir-430c-18 |
| | | 1985 | dre-mir-430c-19 |
| | | 1986 | dre-mir-430c-20 |
| | | 1987 | dre-mir-430c-21 |
| 880 | dre-miR-430i | 1988 | dre-mir-430i-1 |
| | | 1989 | dre-mir-430i-2 |
| | | 1990 | dre-mir-430i-3 |
| 881 | dre-miR-430j | 1991 | dre-mir-430j |
| 882 | hsa-miR-301b | 1992 | hsa-mir-301b |
| 883 | hsa-miR-454 | 1993 | hsa-mir-454 |
| 884 | mmu-miR-721 | 1994 | mmu-mir-721 |
| 885 | dre-miR-454a | 1995 | dre-mir-454a |
| 886 | dre-miR-454b | 1996 | dre-mir-454b |
| 887 | hsa-miR-148b-5p | 1997 | hsa-mir-148b |
| 888 | mmu-miR-322 | 1998 | mmu-mir-322 |
| 889 | rno-miR-322 | 1999 | rno-mir-322 |
| 890 | dre-miR-457a | 2000 | dre-mir-457a |
| 891 | dre-miR-457b | 2001 | dre-mir-457b |
| 892 | hsa-miR-141 | 2002 | hsa-mir-141 |

Table 3-15

| | | | |
|---|---|---|---|
| 893 | mmu-miR-141 | 2003 | mmu-mir-141 |
| 894 | rno-miR-141 | 2004 | rno-mir-141 |
| 895 | mdo-miR-141 | 2005 | mdo-mir-141 |
| 896 | dre-miR-141 | 2006 | dre-mir-141 |
| 897 | hsa-miR-208b | 2007 | hsa-mir-208b |
| 898 | dre-miR-736 | 2008 | dre-mir-736 |
| 899 | hsa-miR-221 | 2009 | hsa-mir-221 |
| 900 | mmu-miR-221 | 2010 | mmu-mir-221 |
| 901 | rno-miR-221 | 2011 | rno-mir-221 |
| 902 | mml-miR-221 | 2012 | mml-mir-221 |
| 903 | ggo-miR-221 | 2013 | ggo-mir-221 |
| 904 | ppa-miR-221 | 2014 | ppa-mir-221 |
| 905 | ppy-miR-221 | 2015 | ppy-mir-221 |
| 906 | mdo-miR-221 | 2016 | mdo-mir-221 |
| 907 | gga-miR-221 | 2017 | gga-mir-221 |
| 908 | xtr-miR-221 | 2018 | xtr-mir-221 |
| 909 | bta-miR-221 | 2019 | bta-mir-221 |
| 910 | dre-miR-221 | 2020 | dre-mir-221 |
| 911 | fru-miR-221 | 2021 | fru-mir-221 |
| 912 | tni-miR-221 | 2022 | tni-mir-221 |
| 913 | hsa-miR-302d* | 967 | hsa-mir-302d |
| 914 | hsa-miR-362-3p | 1020 | hsa-mir-362 |
| 915 | mmu-miR-362-3p | 2023 | mmu-mir-362 |
| 916 | hsa-miR-563 | 2024 | hsa-mir-563 |
| 917 | hsa-miR-449b | 2025 | hsa-mir-449b |
| 918 | mmu-miR-699 | 2026 | mmu-mir-699 |
| 919 | hsa-miR-124 | 2027 | hsa-mir-124-1 |
| | | 2028 | hsa-mir-124-2 |
| | | 2029 | hsa-mir-124-3 |
| 920 | mmu-miR-124 | 2030 | mmu-mir-124-1 |
| | | 2031 | mmu-mir-124-2 |
| | | 2032 | mmu-mir-124-3 |
| 921 | rno-miR-124 | 2033 | rno-mir-124-1 |
| | | 2034 | rno-mir-124-2 |
| | | 2035 | mu-mir-124-3 |
| 922 | dre-miR-124 | 2036 | dre-mir-124-1 |
| | | 2037 | dre-mir-124-2 |
| | | 2038 | dre-mir-124-3 |
| | | 2039 | dre-mir-124-4 |
| | | 2040 | dre-mir-124-5 |
| | | 2041 | dre-mir-124-6 |
| 923 | fru-miR-124 | 2042 | fru-mir-124-1 |
| | | 2043 | fru-mir-124-2 |
| | | 2044 | fru-mir-124-3 |
| 924 | tni-miR-124 | 2045 | tni-mir-124-1 |
| | | 2046 | tni-mir-124-2 |
| | | 2047 | tni-mir-124-3 |
| 925 | hsa-miR-557 | 2048 | hsa-mir-557 |
| 926 | hsa-miR-518a-3p | 2049 | hsa-mir-518a-1 |
| | | 2050 | hsa-mir-518a-2 |
| 927 | hsa-miR-520g | 2051 | hsa-mir-520g |
| 928 | hsa-miR-520d-5p | 1912 | hsa-mir-520d |
| 929 | hsa-miR-520a-5p | 1909 | hsa-mir-520a |
| 930 | hsa-miR-518d-5p | 1069 | hsa-mir-518d |
| 931 | hsa-miR-518e* | 1070 | hsa-mir-518e |
| 932 | hsa-miR-518f* | 1071 | hsa-mir-518f |
| 933 | hsa-miR-519a* | 2052 | hsa-mir-519a-1 |
| | | 2053 | hsa-mir-519a-2 |
| 934 | hsa-miR-519b-5p | 2054 | hsa-mir-519b |
| 935 | hsa-miR-519c-5p | 2055 | hsa-mir-519c |
| 936 | hsa-miR-520c-5p | 1911 | hsa-mir-520c |
| 937 | hsa-miR-522* | 2056 | hsa-mir-522 |
| 938 | hsa-miR-523* | 1073 | hsa-mir-523 |
| 939 | hsa-miR-518a-5p | 2049 | hsa-mir-518a-1 |
| | | 2050 | hsa-mir-518a-2 |
| 2057 | hsa-miR-411 | 2133 | hsa-mir-411 |
| 2058 | hsa-miR-421 | 2134 | hsa-mir-421 |
| 2059 | hsa-miR-425-5p | 2135 | hsa-mir-425 |
| 2060 | hsa-miR-454-5p | 2136 | hsa-mir-454 |
| 2061 | hsa-miR-493-3p | 2137 | hsa-mir-493 |
| 2062 | hsa-miR-532 | 2138 | hsa-mir-532 |
| 2063 | hsa-miR-542-3p | 2139 | hsa-mir-542 |
| 2064 | hsa-miR-545 | 2140 | hsa-mir-545 |
| 2065 | hsa-miR-548a | 2141 | hsa-mir-548a-1 |
| | | 2142 | hsa-mir-548a-2 |
| | | 2143 | hsa-mir-548a-3 |
| 2066 | hsa-miR-548b | 2144 | hsa-mir-548b |
| 2067 | hsa-miR-548d | 2145 | hsa-mir-548d-1 |
| | | 2146 | hsa-mir-548d-2 |
| 2068 | hsa-miR-550 | 2147 | hsa-mir-550-1 |
| | | 2148 | hsa-mir-550-2 |
| 2069 | hsa-miR-551b | 2149 | hsa-mir-551b |

Table 3-16

| | | | |
|---|---|---|---|
| 2070 | hsa-miR-552 | 2150 | hsa-mir-552 |
| 2071 | hsa-miR-553 | 2151 | hsa-mir-553 |
| 2072 | hsa-miR-558 | 2152 | hsa-mir-558 |
| 2073 | hsa-miR-561 | 2153 | hsa-mir-561 |
| 2074 | hsa-miR-565 | 2154 | hsa-mir-565 |
| 2075 | hsa-miR-570 | 2155 | hsa-mir-570 |
| 2076 | hsa-miR-571 | 2156 | hsa-mir-571 |
| 2077 | hsa-miR-579 | 2157 | hsa-mir-579 |
| 2078 | hsa-miR-584 | 2158 | hsa-mir-584 |
| 2079 | hsa-miR-589 | 2159 | hsa-mir-589 |
| 2080 | hsa-miR-591 | 2160 | hsa-mir-591 |
| 2081 | hsa-miR-593 | 2161 | hsa-mir-593 |
| 2082 | hsa-miR-595 | 2162 | hsa-mir-595 |
| 2083 | hsa-miR-599 | 2163 | hsa-mir-599 |
| 2084 | hsa-miR-600 | 2164 | hsa-mir-600 |
| 2085 | hsa-miR-601 | 2165 | hsa-mir-601 |
| 2086 | hsa-miR-604 | 2166 | hsa-mir-604 |
| 2087 | hsa-miR-605 | 2167 | hsa-mir-605 |
| 2088 | hsa-miR-606 | 2168 | hsa-mir-606 |
| 2089 | hsa-miR-609 | 2169 | hsa-mir-609 |
| 2090 | hsa-miR-610 | 2170 | hsa-mir-610 |
| 2091 | hsa-miR-611 | 2171 | hsa-mir-611 |
| 2092 | hsa-miR-614 | 2172 | hsa-mir-614 |
| 2093 | hsa-miR-615 | 2173 | hsa-mir-615 |
| 2094 | hsa-miR-616 | 2174 | hsa-mir-616 |
| 2095 | hsa-miR-617 | 2175 | hsa-mir-617 |
| 2096 | hsa-miR-618 | 2176 | hsa-mir-618 |
| 2097 | hsa-miR-621 | 2177 | hsa-mir-621 |
| 2098 | hsa-miR-624 | 2178 | hsa-mir-624 |
| 2099 | hsa-miR-625 | 2179 | hsa-mir-625 |
| 2100 | hsa-miR-627 | 2180 | hsa-mir-627 |
| 2101 | hsa-miR-630 | 2181 | hsa-mir-630 |
| 2102 | hsa-miR-631 | 2182 | hsa-mir-631 |
| 2103 | hsa-miR-634 | 2183 | hsa-mir-634 |
| 2104 | hsa-miR-637 | 2184 | hsa-mir-637 |
| 2105 | hsa-miR-646 | 2185 | hsa-mir-646 |
| 2106 | hsa-miR-647 | 2186 | hsa-mir-647 |
| 2107 | hsa-miR-648 | 2187 | hsa-mir-648 |
| 2108 | hsa-miR-650 | 2188 | hsa-mir-650 |
| 2109 | hsa-miR-651 | 2189 | hsa-mir-651 |
| 2110 | hsa-miR-653 | 2190 | hsa-mir-653 |
| 2111 | hsa-miR-654 | 2191 | hsa-mir-654 |
| 2112 | hsa-miR-657 | 2192 | hsa-mir-657 |
| 2113 | hsa-miR-661 | 2193 | hsa-mir-661 |
| 2114 | hsa-miR-769-5p | 2194 | hsa-mir-769 |
| 2115 | mmu-miR-411 | 2195 | mmu-mir-411 |
| 2116 | rno-miR-411 | 2196 | rno-mir-411 |
| 2117 | mmu-miR-421 | 2197 | mmu-mir-421 |
| 2118 | xtr-miR-425-5p | 2198 | xtr-mir-425 |
| 2119 | bta-miR-532 | 2199 | bta-mir-532 |
| 2120 | mmu-miR-542-3p | 2200 | mmu-mir-542 |
| 2121 | rno-miR-542-3p | 2201 | rno-mir-542 |
| 2122 | mmu-miR-551b | 2202 | mmu-mir-551b |
| 2123 | mmu-miR-489 | 2203 | mmu-mir-489 |
| 2124 | dre-miR-731 | 2204 | dre-mir-731 |
| 2125 | hsa-miR-493 | 2137 | hsa-mir-493 |
| 2126 | hsa-miR-200o* | 2205 | hsa-mir-200c |
| 2127 | mmu-miR-200o* | 2206 | mmu-mir-200c |
| 2128 | hsa-miR-551a | 2207 | hsa-mir-551a |
| 2129 | mmu-miR-592 | 2208 | mmu-mir-592 |
| 2130 | hsa-miR-373* | 2209 | hsa-mir-373 |
| 2131 | mmu-miR-294* | 2210 | mmu-mir-294 |
| 2132 | hsa-miR-541 | 2211 | hsa-mir-541 |
| 2212 | xtr-miR-1a | 2262 | xtr-mir-1a-1 |
| | | 2263 | xtr-mir-1a-2 |
| 2213 | mmu-miR-7a | 2264 | mmu-mir-7a-1 |
| | | 2265 | mmu-mir-7a-2 |
| 2214 | mmu-miR-10b | 2266 | mmu-mir-10b |
| 2215 | rno-miR-10a-5p | 2267 | rno-mir-10a |
| 2216 | xla-miR-18 | 2268 | xla-mir-18 |
| 2217 | rno-miR-20b-5p | 2269 | rno-mir-20b |
| 2218 | xla-miR-20 | 2270 | xla-mir-20 |
| 2219 | xtr-miR-24a | 2271 | xtr-mir-24a |
| 2220 | xtr-miR-26 | 2272 | xtr-mir-26-1 |
| | | 2273 | xtr-mir-26-2 |
| 2221 | mmu-miR-27a | 2274 | mmu-mir-27a |
| 2222 | mmu-miR-29b | 2275 | mmu-mir-29b-1 |
| | | 2276 | mmu-mir-29b-2 |
| 2223 | mmu-miR-30a | 2277 | mmu-mir-30a |
| 2224 | rno-miR-30b-5p | 2278 | rno-mir-30b |
| 2225 | mmu-miR-302a | 2279 | mmu-mir-302a |
| 2226 | xtr-miR-130c | 2280 | xtr-mir-130c |

Table 3-17

| | | | |
|---|---|---|---|
| 2227 | xtr-miR-133c | 2281 | xtr-mir-133c |
| 2228 | mdo-miR-142 | 2282 | mdo-mir-142 |
| 2229 | dre-miR-142a-3p | 2283 | dre-mir-142a |
| 2230 | mmu-miR-146b | 2284 | mmu-mir-146b |
| 2231 | fru-miR-182 | 2285 | fru-mir-182 |
| 2232 | tni-miR-182 | 2286 | tni-mir-182 |
| 2233 | mmu-miR-193 | 2287 | mmu-mir-193 |
| 2234 | rno-miR-196c | 2288 | rno-mir-196c |
| 2235 | gga-miR-199* | 2289 | gga-mir-199-1 |
| | | 2290 | gga-mir-199-2 |
| 2236 | mmu-miR-202-3p | 2291 | mmu-mir-202 |
| 2237 | tni-miR-204b | 2292 | tni-mir-204b |
| 2238 | xtr-miR-205b | 2293 | xtr-mir-205b |
| 2239 | mmu-miR-208b | 2294 | mmu-mir-208b |
| 2240 | mdo-miR-222a | 2295 | mdo-mir-222a |
| 2241 | mmu-miR-299 | 2296 | mmu-mir-299 |
| 2242 | mmu-miR-299* | 2296 | mmu-mir-299 |
| 2243 | rno-miR-299 | 2297 | rno-mir-299 |
| 2244 | mmu-miR-302o* | 2298 | mmu-mir-302c |
| 2245 | mmu-miR-331-3p | 2299 | mmu-mir-331 |
| 2246 | mmu-miR-340-3p | 2300 | mmu-mir-340 |
| 2247 | rno-miR-340-3p | 2301 | rno-mir-340 |
| 2248 | mmu-miR-342-3p | 2302 | mmu-mir-342 |
| 2249 | rno-miR-342-3p | 2303 | rno-mir-342 |
| 2250 | mmu-miR-362-5p | 2023 | mmu-mir-362 |
| 2251 | rno-miR-376b-3p | 2304 | rno-mir-376b |
| 2252 | mmu-miR-378* | 2305 | mmu-mir-378 |
| 2253 | rno-miR-378* | 2306 | rno-mir-378 |
| 2254 | mmu-miR-423-3p | 2307 | mmu-mir-423 |
| 2255 | mmu-miR-449a | 2308 | mmu-mir-449a |
| 2256 | mmu-miR-455* | 2309 | mmu-mir-455 |
| 2257 | mmu-miR-483* | 2310 | mmu-mir-483 |
| 2258 | mmu-miR-485 | 2311 | mmu-mir-485 |
| 2259 | rno-miR-485 | 2312 | rno-mir-485 |
| 2260 | mmu-miR-488* | 2313 | mmu-mir-488 |
| 2261 | mmu-miR-501-5p | 2314 | mmu-mir-501 |
| 2315 | mmu-miR-425 | 2322 | mmu-mir-425 |
| 2316 | rno-miR-425 | 2323 | rno-mir-425 |
| 2317 | mmu-miR-493 | 2324 | mmu-mir-493 |
| 2318 | rno-miR-493 | 2325 | rno-mir-493 |
| 2319 | mmu-miR-532-5p | 2326 | mmu-mir-532 |
| 2320 | rno-miR-532-5p | 2327 | rno-mir-532 |
| 2321 | mmu-miR-615-3p | 2328 | mmu-mir-615 |

In the present invention, a nucleic acid having an identity of 90% or more to a nucleotide sequence shown by any one of SEQ ID NO:1-2328 means a nucleic acid having an identity of at least 90% or more, preferably 93% or more, more preferably 95% or more, still more preferably 96% or more, particularly preferably 97% or more, most preferably 98% or more, to a nucleic acid consisting of a nucleotide sequence shown by any one of SEQ ID NO:1-2328, as calculated using an analytical software program such as BLAST [J. Mol. Biol., 215, 403 (1990)] or FASTA [Methods in Enzymology, 183, 63 (1990)].

The stringent conditions in the above refer to those under which signals can be detected by adding a membrane having one chain blotted and the other chain labeled with ³²P-ATP to a Hybridization buffer consisting of 7.5 mL, 0.6 mL of 1 M Na₂HPO₄ (pH 7.2), 21 mL of 10% SDS, 0.6 mL of 50×Denhardt's solution, and 0.3 mL of 10 mg/mL sonicated salmon sperm DNA, carrying out a reaction at 50° C. overnight, thereafter washing the membrane with 5×SSC/5% SDS liquid at 50° C. for 10 minutes, and further washing the same with 1×SSC/1% SDS solution at 50° C. for 10 minutes, thereafter taking out the membrane, and exposing it to an X-ray film.

The method of detecting the expression of nucleic acid such as a miRNA using a nucleic acid may be any method, as far as the presence of nucleic acid such as a miRNA or miRNA precursor in a sample can be detected; for example, (1) Northern hybridization, (2) dot blot hybridization, (3) in situ hybridization, (4) quantitative PCR, (5) differential hybridization, (6) microarray, (7) ribonuclease protection assay and the like can be mentioned.

The method of detecting a mutation of nucleic acid such as a miRNA using a nucleic acid may be any method, as far as it enables detection of a mutation of the nucleotide sequence of nucleic acid such as a miRNA or miRNA precursor in a sample; for example, a method wherein a hetero-double-strand formed by hybridization of a nucleic acid having a non-mutated nucleotide sequence and a nucleic acid having a mutated nucleotide sequence are detected, or a method wherein a sample-derived nucleotide sequence is directly sequenced to detect the presence or absence of a mutation and the like can be mentioned.

A vector that expresses a nucleic acid may be any vector, as far as it is designed to biosynthesize a nucleic acid such as miRNA when introduced to, and transcribed in, a cell. Specifically, as examples of vectors capable of expressing a nucleic acid such as miRNA in a cell, pcDNA6.2-GW/miR (manufactured by Invitrogen), pSilencer 4.1-CMV (manufactured by Ambion), pSINsi-hH1 DNA (manufactured by Takara Bio Inc.), pSINsi-hU6 DNA (manufactured by Takara Bio Inc.), pENTR/U6 (manufactured by Invitrogen) and the like can be mentioned.

The method of suppressing the expression of a gene having a target nucleotide sequence of nucleic acid such as miRNA (hereinafter to be referred to as target gene) may be any method, as far as it suppresses the expression of a gene having a target nucleotide sequence by means of the activity to suppress the expression of a mRNA having the target nucleotide sequence of nucleic acid such as miRNA. Here, to suppress the expression encompasses a case where the translation of a mRNA is suppressed, and a case where cleavage or degradation of a mRNA results in a decreased amount of protein translated from the mRNA. Specifically, as substances that suppress the expression of a mRNA having the target nucleotide sequence, nucleic acids such as an siRNA and an antisense oligonucleotides can be mentioned. The siRNA can be prepared on the basis of information on the continuous sequence of the mRNA [Genes Dev., 13, 3191 (1999)]. The number of nucleotide residues constituting one strand of the siRNA is preferably 17 to 30 residues, more preferably 18 to 25 residues, still more preferably 19 to 23 residues.

MiRNAs include artificial miRNAs that are single-stranded RNAs 17 to 28 nucleotides long comprising as the 2nd to 8th nucleotides a sequence that is complementary to a consecutive, 7-nucleotide sequence present in the 3′-untranslational region of the mRNA of the target gene. In the case of an RNA comprising a sequence extended anteriorly and posteriorly that forms a hairpin structure, wherein the miRNA sequence can be cut out in cells from either one strand of the hairpin structure by the biosynthetic pathway of the miRNA, the extended sequence is called an artificial miRNA precursor. With a gene whose expression is to be suppressed as the target gene, an artificial miRNA and artificial miRNA precursor can be designed using the method described above.

A target nucleotide sequence of a nucleic acid such as miRNA refers to a nucleotide sequence consisting of several nucleotides recognized by a nucleic acid such as miRNA and the like of the present invention, wherein the expression of a mRNA having the nucleotide sequence is suppressed by a nucleic acid such as miRNA of the present invention. Because a mRNA having a nucleotide sequence complementary to the 2nd to 8th nucleotides on the 5′ terminal side of a miRNA undergoes suppression of the translation thereof by the miRNA [Current Biology, 15, R458-R460 (2005)], a nucleotide sequence complementary to the nucleotide sequence of the 2nd to 8th nucleotides on the 5′ terminal side of a nucleic acid such as miRNA of the present invention can be mentioned as a target nucleotide sequence. For example, by providing a target sequence complementary to the 2nd to 8th nucleotides on the 5′ terminal side of a miRNA, and selecting a mRNA comprising a sequence completely identical to a set of 3′ UTR nucleotide sequences of human mRNAs by a method such as character string search, the target nucleotide sequence can be determined. A set of 3′ UTR nucleotide sequences of human mRNAs can be prepared using information on genomic sequences and gene positions that can be acquired from “UCSC Human Genome Browser Gateway (http://genome.ucsc.edu/cgi-bin/hgGateway)”. As specific examples of genes having a target nucleotide sequence of miRNA consisting of the nucleotide sequence shown by any of SEQ ID NOs: 1-939, 2057-2132, 2212-2261 and 2315-2321, the genes shown in Table 4 (Table 4-1 to Table 4-157), represented by names (Official Symbols and Gene IDs) used in the EntreGene database (http://www.ncbi.nlm.nih.gov/Entrez/) of US National 30 Center for Biotechnology Information (NCBI), can be mentioned.

| | | |
|---|---|---|
| Name of | | |
| representive miRNA | SEQ ID NO: | target gene |

Table 4-1

| | | |
|---|---|---|
| hsa-miR-1 | 1; 130; 131; | CDC42(998); RCC1(1104); RBBP4(5928); CDC20(991); KIAA0467(23334); |
| hsa-miR-206 | 132; 133; 134; | SH3GLB1(51100); PROK1(84432); CD2(914); BCL9(607); BNIPL(149428); |
| | 135; 136; | CKS1B(1163); PEA15(8682); UHMK1(127933); IL24(11009); C1orf95 |
| | 137; 138; | (375057); YARS(8565); CLSPN(63967); TESK2(10420); SASS6(163786); NOTCH2 |
| | 139; 2; 140; | (4853); PYGO2(90780); DEDD(9191); RHOB(388); BIRC6(57448); |
| | 141; 142; 143; | PRKCE(5581); KCNIP3(30818); TAIP-2(80034); CASP10(843); CASP8(841); |
| | 144; 145; | FZD7(8324); CD28(940); COL4A3(1285); SEPT2(4735); YWHAQ(10971); |
| | 146; 147; | CARD12(58484); ANAPC1(64682); TLK1(9874); FRZB(2487); NCKAP1 |
| | 803; 2212; | (10787); PAX3(5077); HDAC4(9759); FANCD2(2177); VHL(7428); RBM5 |
| | | (10181); GNL3(26354); FAIM(55179); KNG1(3827); CIDEC(63924); RAF1 |
| | | (5894); UBE1C(9039); PIK3CB(5291); ATR(545); CCNG2(901); CYP2U1 |
| | | (113612); APBB2(323); UNC5C(8633); SFRP2(6423); NIPBL(25836); RNUXA |
| | | (51808); DAP(1611); MCC(4163); SEMA6A(57556); IRF1(3659); CDC25C |
| | | (995); PFDN1(5201); VEGF(7422); UST(10090); NEDD9(4739); CCNC |
| | | (892); BCLAF1(9774); LATS1(9113); PDCD2(5134); AHR(196); ADCY1 |
| | | (107); EGFR(1956); HBP1(26959); CNTNAP2(26047); CUL1(8454); PMS2 |
| | | (5395); ELMO1(9844); IGFBP3(3486); CDK6(1021); BNIP3L(665); FZD3(7976); |
| | | BAG4(9530); SULF1(23213); E2F5(1875); FZD6(8323); EBAG9(9166); |
| | | WISP1(8840); PINX1(54984); TNFRSF10B(8795); TRIM35(23087); SFRP1 |
| | | (6422); STK3(6788); UNC13B(10497); CDK9(1025); CDKN2B(1030); |
| | | SEMA4D(10507); PTCH(5727); TNFSF15(9966); TNFSF8(944); TNKS2 |
| | | (80351); LZTS2(84445); BTRC(8945); TCF7L2(6934); BAG3(9531); DOCK1 |
| | | (1793); MKI67(4288); WEE1(7465); GAS2(2620); HIPK3(10114); API5(8539); |
| | | LRP5(4041); LOH11CR2A(4013); BDNF(627); WT1(7490); SHANK2 |
| | | (22941); FZD4(8322); SESN3(143686); ZW10(9183); BCL9L(283149); CCND2 |
| | | (894); TNFRSF7(939); CRADD(8738); OAS2(4939); BCAT1(586); KRAS |
| | | (3845); CDK4(1019); ARPC3(10094); MPHOSPH9(10198); CHFR(55743); |
| | | HSMPP8(54737); RB1(5925); LATS2(26524); BCL2L2(599); KIAA0323 |
| | | (23351); TXNDC(81542); TRAF3(7187); CDKL1(8814); DLG7(9787); LTBP2 |
| | | (4053); KLF13(51621); BUB1B(701); PTPLAD1(51495); PAQR5(54852); |
| | | IGF1R(3480); AVEN(57099); BMF(90427); BNIP2(663); CSNK1G1(53944); |
| | | CYLD(1540); KATNB1(10300); CTCF(10664); NFATC3(4775); AXIN1 |
| | | (8312); MAPK3(5595); FTS(64400); TERF2(7014); AP1G1(164); PAFAH1B1 |
| | | (5048); MAPK7(5598); CCL2(6347); LIG3(3980); LASP1(3927); NGFR |
| | | (4804); TLK2(11011); PRKCA(5578); SPHK1(8877); MNT(4335); GAS7(8522); |
| | | BRCA1(672); PTRH2(51651); C17orf62(79415); CABLES1(91768); MAPK4 |
| | | (5596); PHLPP(23239); ROCK1(6093); ZNF24(7572); BCL2(596); CHAF1A |
| | | (10036); ARHGEF18(23370); PSMD8(5714); AXL(558); APOE(348); |
| | | FOSB(2354); SPHK2(56848); NALP2(55655); DAPK3(1613); ZNF571 |
| | | (51276); DPF1(8193); CARD8(22900); KLK10(5655); CEP250(11190); STK4 |
| | | (6789); BAK1(578); AURKA(6790); CTCFL(140690); SON(6651); BCL2L13 |
| | | (23786); SMARCB1(6598); GAS2L1(10634); SEPT3(55964); MAPK1(5594); |
| | | PPM1F(9647); CARD10(29775); SMC1L2(27127); CCNB3(85417); ARSD |
| | | (414); BTK(695); SEPT6(23157); HCFC1(3054); G6PD(2539); |
| hsa-miR-7 | 3; 148; 149; | SAMD11(148398); TP73(7161); PIK3CD(5293); NBL1(4681); SFN(2810); |
| | 150; 151; 152; | RCC1(1104); MACF1(23499); SH3GLB1(51100); RAP1A(5906); BCL9(607); |
| | 153; 154; | UHMK1(127933); MDM4(4194); TRAF5(7188); TGFB2(7042); FRAP1 |
| | 155; 156; | (2475); CASP9(842); ZBTB17(7709); RCC2(55920); NOTCH2(4853); MCL1 |
| | 157; 158; 159; | (4170); PYGO2(90780); DEDD(9191); ASPM(259266); DDEF2(8853); BIRC6 |
| | 160; 161; | (57448); ZAK(51776); CASP10(843); ALS2CR19(117583); INHA(3623); |
| | 162; 163; | COL4A3(1285); SEPT2(4735); CCT4(10575); TGFA(7039); EDAR(10913); |
| | 164; 165; 166 | SEPT10(151011); BUB1(699); IL1B(3553); BIN1(274); MCM6(4175); FZD5 |
| | 167; 2213; | (7855); DTYMK(1841); FANCD2(2177); CRTAP(10491); TDGF1(6997); |
| | | GNL3(26354); IL17RB(55540); TADA3L(10474); RAF1(5894); NME6(10201); |
| | | NEK4(6787); GSK3B(2932); HD(3064); CD38(952); SEPT11(55752); FGF5 |
| | | (2250); PLK4(10733); TLR2(7097); GAK(2580); BTC(685); CCNI(10983); |
| | | SNCA(6622); DKK2(27123); LEF1(51176); CASP6(839); SETD7(80854); |
| | | NEK1(4750); PDCD6(10016); FLJ11193(55322); APC(324); CSNK1G3 |
| | | (1456); FGF10(2255); ATG12(9140); SEMA6A(57556); CD74(972); DDX41 |
| | | (51428); LTA(4049); TNF(7124); PARC(23113); CDC5L(988); TTK(7272); NEDD9 |
| | | (4739); MDC1(9656); CCND3(896); DST(667); PLAGL1(5325); EGFR |
| | | (1956); FZD9(8326); STAG3(10734); BCAP29(55973); CUL1(8454); ATP6V0E2L |
| | | (155066); CARD4(10392); INHBA(3624); BRAF(673); BNIP3L(6 |

Table 4-2

| | | |
|---|---|---|
| | | 65); PTK2B(2185); UNC5D(137970); BAG4(9530); TACC1(6867); MYC(4609); |
| | | LZTS1(11178); BIN3(55909); ADRA1A(148); SFRP1(6422); JAK2(3717); |
| | | RECK(8434); NTRK2(4915); DAPK1(1612); C9orf47(286223); ABL1(25); |
| | | SEMA4D(10507); PTCH(5727); AKNA(80709); ANAPC2(29882); SIRT1 |
| | | (23411); SGPL1(8879); PTEN(5728); KIF11(3832); BTRC(8945); SUFU(51684); |
| | | DOCK1(1793); SFRP5(6425); SMNDC1(10285); DCLRE1A(9937); |
| | | INCENP(3619); LGALS12(85329); CCND1(595); CUL5(8065); ATM(472); |
| | | CBL(867); HRASLS3(11145); BAD(572); RELA(5970); BRMS1(25855); CLCF1 |
| | | (23529); PPP1CA(5499); PAK1(5058); FZD4(8322); PPP2R1B(5519); |
| | | CCND2(894); CNAP1(9918); TEGT(7009); SP1(6667); FZD10(11211); RAN |
| | | (5901); FGF6(2251); BCAT1(586); HDAC7A(51564); MCRS1(10445); ATF7 |
| | | (11016); PAWR(5074); ANAPC7(51434); MPHOSPH9(10198); BRCA2 |
| | | (675); RB1(5925); LATS2(26524); KIAA0247(9766); PSEN1(5663); VASH1 |
| | | (22846); TRAF3(7187); CIDEB(27141); STRN3(29966); EGLN3(112399); CDKL1 |
| | | (8814); CHES1(1112); AKT1(207); ZNF609(23060); KIF23(9493); HMG20A |
| | | (10363); IGF1R(3480); SLC12A6(9990); BMF(90427); PYGO1(26108); |
| | | TSC2(7249); DNAJA3(9093); CARD15(64127); KATNB1(10300); CDK10 |
| | | (8558); PKMYT1(9088); GSPT1(2935); SEPT1(1731); SLC12A4(6560); |
| | | HIC1(3090); PAFAH1B1(5048); GSG2(83903); TNFSF12(8742); MAPK7 |
| | | (5598); NLK(51701); NF1(4763); LIG3(3980); MAPT(4137); PPM1D(8493); |
| | | TLK2(11011); WDR68(10238); CARD14(79092); P2RX1(5023); GAS7(8522); |
| | | TIAF1(9220); GAS2L2(246176); BECN1(8678); BRCA1(672); HDAC5 |
| | | (10014); PHB(5245); MAFG(4097); MAPRE2(10982); MAPK4(5596); BCL2 |
| | | (596); UHRF1(29128); TNFSF9(8744); PIK3R2(5296); MAP3K10(4294); |
| | | AXL(558); BCL2L12(83596); ATF5(22809); DAPK3(1613); ERF(2077); MCM8 |
| | | (84515); PLCB1(23236); CEP250(11190); CSE1L(1434); RASSF2(9770); |
| | | BCL2L1(598); E2F1(1869); SPATA2(9825); CTCFL(140690); CABLES2 |
| | | (81928); DIDO1(11083); SLC5A3(6526); ITGB2(3689); SEPT5(5413); NF2 |
| | | (4771); MAPK1(5594); PPM1F(9647); TAF1(6872); FIGF(2277); SMC1L1 |
| | | (8243); SEPT6(23157); CUL4B(8450); |
| hsa-miR-10a | 4; 168; 169; | DFFB(1677); RBBP4(5928); MACF1(23499); NFYC(4802); CDC14A(8556); |
| | 170; 171; 172; | PEA15(8682); FASLG(356); MDM4(4194); IL19(29949); HES2(54626); TNFRSF9 |
| | 173; 174; | (3604); CTNNBIP1(56998); FRAP1(2475); CASP9(842); CLCC1 |
| | 175; 176; | (23155); DEDD(9191); ASPM(259266); TNNI1(7135); PRKCE(5581); CCT7 |
| | 177; 178; 179; | (10574); HK2(3099); KCNIP3(30818); ZAK(51776); CFLAR(8837); CASP8 |
| | 180; 181; | (841); INHA(3623); SEPT2(4735); YWHAQ(10971); PPM1G(5496); EIF2AK2 |
| | 182; 183; | (5610); ERCC3(2071); HDAC4(9759); EDEM1(9695); FANCD2(2177); |
| | 184; 185; 186; | NR2C2(7182); PDCD6IP(10015); CCBP2(1238); RNF7(9616); SMC4L1(10051); |
| | 187; 188; | PSMD2(5708); ZNF445(353274); RASSF1(11186); NEK4(6787); WHSC1 |
| | 189; 190; | (7468); LOC285429(285429); SNCA(6622); LEF1(51176); SFRP2(6423); |
| | 191; 192; 804; | ARSK(153642); RAD50(10111); PRLR(5618); BIRC1(4671); MCC(4163); |
| | 2214; 2215; | ATG12(9140); NRG2(9542); E2F3(1871); MAPK13(5603); PARC(23113); |
| | | FOXO3A(2309); SASH1(23328); DST(667); LATS1(9113); PDCD2(5134); |
| | | SEPT7(989); FZD9(8326); FZD1(8321); STAG3(10734); CASP2(835); PMS2 |
| | | (5395); CYCS(54205); CARD4(10392); CDK6(1021); ZC3HC1(51530); |
| | | HIPK2(28996); PTK2B(2185); UNC5D(137970); LZTS1(11178); TNFRSF10B |
| | | (8795); TNFRSF10D(8793); TRIM35(23087); CLU(1191); TNFRSF11B |
| | | (4982); ABL1(25); CDKN2A(1029); TAF1L(138474); BAG1(573); SEMA4D |
| | | (10507); FANCC(2176); TNFSF8(944); TRAF1(7185); TSC1(7248); ZMYND11 |
| | | (10771); UNC5B(219699); PTEN(5728); BTRC(8945); SUFU(51684); |
| | | PDCD11(22984); DOCK1(1793); SFMBT2(57713); C1orf111(221060); |
| | | ZWINT(11130); PPP3CB(5532); DLG5(9231); MKI67(4288); TUB(7275); |
| | | WEE1(7465); HIPK3(10114); DPF2(5977); ATM(472); TAGLN(6876); WT1 |
| | | (7490); FZD4(8322); SESN3(143686); PPP2R1B(5519); BCL9L(283149); |
| | | BCL2L14(79370); FLJ20489(55652); MDM2(4193); CRADD(8738); APAF1 |
| | | (317); FAIM2(23017); PAWR(5074); DUSP6(1848); DIP13B(55198); CDK2AP1 |
| | | (8099); SCARB1(949); GOLGA3(2802); RB1(5925); RFP2(10206); VASH1 |
| | | (22846); LTBP2(4053); BAG5(9529); NDN(4692); BNIP2(663); DAPK2 |
| | | (23604); CSNK1G1(53944); PAPD5(64282); NME3(4832); ERN2(10595); |
| | | CIAPIN1(57019); APPBP1(8883); SMPD3(55512); PAFAH1B1(5048); CDC6 |
| | | (990); NME2(4831); PRKCA(5578); P2RX1(5023); NALP1(22861); SEPT4 |
| | | (5414); FAM33A(348235); CETN1(1068); CCDC5(115106); COLEC12 |
| | | (81035); BCL2(596); DNM2(1785); UBA52(7311); AXL(558); APOE(348); BCL2L12 |
| | | (83596); NALP2(55655); TNFSF14(8740); CARD8(22900); SIGLEC11 |
| | | (114132); KLK10(5655); MAPRE1(22919); STK4(6789); PARD6B(84 |

Table 4-3

| | | |
|---|---|---|
| | | 612); BCL2L1(598); BTBD4(140685); HUNK(30811); SON(6651); NF2(4771); |
| | | MCM5(4174); LOC63929(63929); SEPT3(55964); YPEL1(29799); MAPK1 |
| | | (5594); PPM1F(9647); NHP2L1(4809); CCNB3(85417); TAF1(6872); |
| | | BIRC4(331); DKC1(1736); SMC1L1(8243); SPIN2(54466); |
| hsa-miR-18a | 5; 193; 194; | DFFB(1677); CDC42(998); LCK(3932); MACF1(23499); SH3GLB1(51100); |
| hsa-miR-18b | 195; 196; 197; | RAP1A(5906); BNIPL(149428); UHMK1(127933); TRAF5(7188); CIAS1 |
| | 198; 199; | (114548); TNFRSF9(3604); CTNNBIP1(56998); DFFA(1676); ZBTB17(7709); |
| | 200; 201; | YARS(8565); MUTYH(4595); FAF1(11124); NOTCH2(4853); ASPM(259266); |
| | 202; 203; 204; | CCT7(10574); PDCL3(79031); PROC(5624); TAIP-2(80034); ZAK |
| | 205; 206; | (51776); CASP10(843); CASP8(841); CAB39(51719); PPM1G(5496); CARD12 |
| | 207; 208; | (58484); SEPT10(151011); ANAPC1(64682); IL1A(3552); IL1B(3553); BIN1 |
| | 209; 210; 211; | (274); IFIH1(64135); STAT1(6772); CLK1(1195); CTNNB1(1499); HD |
| | 212; 213; | (3064); CCNG2(901); UNC5C(8633); DKK2(27123); LEF1(51176); APC(324); |
| | 6; 214; 215; | UNC5A(90249); PRKAA1(5562); NRG2(9542); HDAC3(8841); DDX41 |
| | 216; 217; | (51428); ALDH5A1(7915); TCF19(6941); MAPK13(5603); PARC(23113); TNFAIP3 |
| | 218; 2216; | (7128); NEDD9(4739); MDC1(9656); IHPK3(117283); DST(667); |
| | | PLAGL1(5325); AHR(196); HDAC9(9734); SEPT7(989); STK17A(9263); ADCY1 |
| | | (107); FZD1(8321); CASP2(835); INHBA(3624); FASTK(10922); TNFRSF10C |
| | | (8794); ESCO2(157570); E2F5(1875); DENND3(22898); LZTS1 |
| | | (11178); TNFRSF10B(8795); TNFRSF10D(8793); ANKRD15(23189); UNC13B |
| | | (10497); MGC21881(389741); DAPK1(1612); CDK9(1025); ABL1(25); |
| | | IFNA2(3440); CDKN2B(1030); TAF1L(138474); FANCG(2189); PTCH(5727); |
| | | TNFSF15(9966); TNFSF8(944); TSC1(7248); ANAPC2(29882); DKK1 |
| | | (22943); SGPL1(8879); UNC5B(219699); FZD8(8325); KCNMA1(3778); |
| | | DLG5(9231); FBXW4(6468); TIAL1(7073); MKI67(4288); BNIP3(664); STIM1 |
| | | (6786); WEE1(7465); INCENP(3619); LGALS12(85329); SIPA1(6494); |
| | | ATM(472); FGF3(2248); FZD4(8322); CASP4(837); BTG4(54766); ZW10 |
| | | (9183); BCL9L(283149); H2AFX(3014); CCND2(894); LTBR(4055); ESPL1 |
| | | (9700); PDE1B(5153); CDK2(1017); CCT2(10576); GAS2L3(283431); HDAC7A |
| | | (51564); WIF1(11197); ANAPC7(51434); HRK(8739); MPHOSPH9 |
| | | (10198); SCARB1(949); CHFR(55743); HSMPP8(54737); RFP2(10206); LIG4 |
| | | (3981); BCL2L2(599); PSEN1(5663); DAD1(1603); LTBP2(4053); TGFB3 |
| | | (7043); CHES1(1112); MOAP1(64112); PPP1R13B(23368); AKT1(207); |
| | | MAPK6(5597); SLC12A6(9990); BCL2L10(10017); BNIP2(663); CA12(771); |
| | | CSNK1G1(53944); CCNF(899); KREMEN2(79412); RBL2(5934); NME3 |
| | | (4832); PKMYT1(9088); GSPT1(2935); DNAJA2(10294); FTS(64400); APPBP1 |
| | | (8883); TRADD(8717); SPHK1(8877); MNT(4335); GAS7(8522); LOC201181 |
| | | (201181); HDAC5(10014); ERN1(2081); SOCS3(9021); CSNK1D |
| | | (1453); MAPK4(5596); HMG20B(10362); PIK3R2(5296); UBA52(7311); FOSB |
| | | (2354); SPHK2(56848); TNFSF14(8740); SIRT2(22933); CGB7(94027); |
| | | KLK10(5655); CTNNBL1(56259); STK4(6789); PARD6B(84612); BIRC7 |
| | | (79444); CSNK2A1(1457); RBL1(5933); PTPRT(11122); USP16(10600); |
| | | SON(6651); BTG3(10950); SNF1LK(150094); GAS2L1(10634); EP300(2033); |
| | | PPM1F(9647); MN1(4330); CARD10(29775); SMC1L2(27127); HDAC6 |
| | | (10013); TAF1(6872); IKBKG(8517); DKC1(1736); PPP2R3B(28227); BTK |
| | | (695); MTCP1(4515); |
| hsa-miR-18a* | 7; 219; | KCNAB2(8514); TNFRSF1B(7133); NBL1(4681); RCC1(1104); LCK(3932); |
| | | HDAC1(3065); MACF1(23499); CDC7(8317); PROK1(84432); RAP1A(5906); |
| | | BCL9(607); CKS1B(1163); PEA15(8682); IL24(11009); C1orf69(200205); |
| | | CLAS1(114548); TNFRSF14(8764); TNFRSF9(3604); DFFA(1676); FRAP1 |
| | | (2475); MAD2L2(10459); ZBTB17(7709); RCC2(55920); E2F2(1870); |
| | | KIAA0319L(79932); CLSPN(63967); MUTYH(4595); ITGB3BP(23421); |
| | | GFI1(2672); NOTCH2(4853); PYGO2(90780); ASPM(259266); TNNI1(7135); |
| | | TP53BP2(7159); RHOB(388); BIRC6(57448); HTRA2(27429); HK2(3099); |
| | | BRRN1(23397); BCL2L11(10018); RIF1(55183); ZAK(51776); CFLAR |
| | | (8837); CASP10(843); COL4A3(1285); PSMD1(5707); CGREF1(10669); TIA1 |
| | | (7072); EDAR(10913); HDAC4(9759); KIF1A(547); OGG1(4968); VHL |
| | | (7428); PDCD6IP(10015); GNL3(26354); ACPP(55); CIDEC(63924); RAF1 |
| | | (5894); AXUD1(64651); TRAIP(10293); NEK4(6787); PIK3CB(5291); TFDP2 |
| | | (7029); HD(3064); TLR2(7097); APBB2(323); UNC5C(8633); LEF1(51176); |
| | | NEK1(4750); CARD6(84674); RAD17(5884); CSNK1G3(1456); TCF7 |
| | | (6932); UNC5A(90249); N4BP3(23138); DAP(1611); GDNF(2668); MCC(4163); |
| | | ATG12(9140); IRF1(3659); FBXW11(23291); CDKN1A(1026); PARC |
| | | (23113); VEGF(7422); PTP4A1(7803); FOXO3A(2309); SASH1(23328); DHX16 |
| | | (8449); MDC1(9656); IHPK3(117283); CUL7(9820); DST(667); SESN1 |

Table 4-4

| | | |
|---|---|---|
| | | (27244); SGK(6446); BCLAF1(9774); LATS1(9113); TTYH3(80727); SEPT7 |
| | | (989); ADCY1(107); FZD1(8321); STAG3(10734); RINT1(60561); CASP2 |
| | | (835); MAD1L1(8379); SNX8(29886); TRIAD3(54476); CYCS(54205); CARD4 |
| | | (10392); ELMO1(9844); SFRP4(6424); INHBA(3624); ZC3HC1(51530); |
| | | HIPK2(28996); TNFRSF10C(8794); UNC5D(137970); BAG4(9530); TACC1 |
| | | (6867); E2F5(1875); MYC(4609); LZTS1(11178); TNFRSF10B(8795); TNFRSF10D |
| | | (8793); TNFRSF10A(8797); DUSP4(1846); UNC13B(10497); |
| | | NTRK2(4915); DAPK1(1612); CDK9(1025); ABL1(25); CDKN2A(1029); TAF1L |
| | | (138474); BAG1(573); CTNNAL1(8727); TNFSF8(944); TRAF1(7185); |
| | | CIZ1(25792); TSC1(7248); CARD9(64170); ANAPC2(29882); SGPL1(8879); |
| | | UNC5B(219699); PCDH21(92211); LZTS2(84445); SUFU(51684); PDCD11 |
| | | (22984); CSPG6(9126); BAG3(9531); CXCL12(6387); ZWINT(11130); |
| | | AMID(84883); DLG5(9231); DCLRE1A(9937); MKI67(4288); WEE1(7465); |
| | | HIPK3(10114); INCENP(3619); LGALS12(85329); SIPA1(6494); RPS6KB2 |
| | | (6199); LRP5(4041); CCND1(595); BIRC3(330); CUL5(8065); ATM(472); |
| | | CHEK1(1111); APBB1(322); DKK3(27122); WT1(7490); MAP3K11(4296); |
| | | BRMS1(25855); CLCF1(23529); MTL5(9633); FGF4(2249); GAB2(9846); |
| | | PPP2R1B(5519); BCL9L(283149); H2AFX(3014); FKBP4(2288); CCND2 |
| | | (894); TNFRSF7(939); PTPN6(5777); CDKN1B(1027); PDE1B(5153); |
| | | KRAS(3845); MCRS1(10445); FAIM2(23017); WIF1(11197); PAWR(5074); |
| | | ANAPC7(51434); MPHOSPH9(10198); HSMPP8(54737); TNFRSF19(55504); |
| | | CUL4A(8451); TFDP1(7027); BCL2L2(599); VASH1(22846); TRAF3 |
| | | (7187); ACIN1(22985); GZMH(2999); GZMB(3002); EGLN3(112399); SPTB |
| | | (6710); LTBP2(4053); PGF(5228); NEK9(91754); TGFB3(7043); CHES1 |
| | | (1112); BAG5(9529); PPP1R13B(23368); AKT1(207); JAG2(3714); BUB1B |
| | | (701); FGF7(2252); AVEN(57099); BMF(90427); CSNK1G1(53944); TSC2 |
| | | (7249); TRAF7(84231); CCNF(899); PLK1(5347); SBK1(388228); RBL2(5934); |
| | | ELMO3(79767); CDK10(8558); AXIN1(8312); PKMYT1(9088); ERN2 |
| | | (10595); MAPK3(5595); CIAPIN1(57019); CSNK2A2(1459); TRADD(8717); |
| | | SLC12A4(6560); SMPD3(55512); HIC1(3090); ALOX15B(247); MAPK7 |
| | | (5598); SARM1(23098); TRAF4(9618); NF1(4763); MLLT6(4302); STARD3 |
| | | (10948); RND2(8153); FZD2(2535); MAPT(4137); NME1(4830); CDK3 |
| | | (1018); SPHK1(8877); CBX2(84733); CARD14(79092); BAIAP2(10458); MNT |
| | | (4335); P2RX1(5023); NALP1(22861); TP53(7157); GAS7(8522); HCP1 |
| | | (113235); GAS2L2(246176); BRCA1(672); SEPT4(5414); LIMD2(80774); |
| | | ERN1(2081); SOCS3(9021); PSCD1(9267); CBX4(8535); TBC1D16(125058); |
| | | CSNK1D(1453); CIDEA(1149); CABLES1(91768); DCC(1630); PHLPP |
| | | (23239); BCL2(596); CSNK1G2(1455); FZR1(51343); HMG20B(10362); PIN1 |
| | | (5300); DNM2(1785); PIK3R2(5296); UBA52(7311); APLP1(333); PSMD8 |
| | | (5714); AXL(558); ATF5(22809); MATK(4145); CDC37(11140); CDKN2D |
| | | (1032); PRX(57716); ERF(2077); PPP1R13L(10848); BBC3(27113); KLK10 |
| | | (5655); CEP250(11190); CTNNBL1(56259); STK4(6789); BCL2L1(598); |
| | | E2F1(1869); SYCP2(10388); CABLES2(81928); DIDO1(11083); USP16(10600); |
| | | SON(6651); CHAF1B(8208); SNF1LK(150094); ITGB2(3689); BCL2L13 |
| | | (23786); ADORA2A(135); KREMEN1(83999); NF2(4771); MTP18(51537); |
| | | SYNGR1(9145); ZC3H7B(23264); BIK(638); MAPK1(5594); PPM1F |
| | | (9647); MN1(4330); KIAA1904(114794); CARD10(29775); CBX7(23492); |
| | | PDGFB(5155); TOB2(10766); NHP2L1(4809); MAPK12(6300); PCTK1(5127); |
| | | AGTR2(186); BIRC4(331); SMC1L1(8243); SPIN2(54466); FAM58A |
| | | (92002); HCFC1(3054); |
| hsa-miR-20b | 8; 220; 221; | PRKCZ(5590); TNFRSF1B(7133); NBL1(4681); NUDC(10726); SESN2(83667); |
| hsa-miR-106a | 222; 223; 224; | RBBP4(5928); MACF1(23499); SH3GLB1(51100); CDC7(8317); RAP1A |
| hsa-miR-106b | 225; 226; | (5906); BCL9(607); BNIPL(149428); DAP3(7818); UHMK1(127933); FASLG |
| | 227; 228; | (356); MDM4(4194); TGFB2(7042); CIAS1(114548); TNFRSF9(3604); |
| | 229; 230; 231; | DFFA(1676); E2F2(1870); SASS6(163786); MCL1(4170); PYGO2(90780); |
| | 232; 233; | ISG20L2(81875); DEDD(9191); IL10(3586); MAPRE3(22924); BIRC6 |
| | 234; 235; | (57448); TCF7L1(83439); BCL2L11(10018); RIF1(55183); TAIP-2(80034); |
| | 236; 237; 238; | CYBRD1(79901); ZAK(51776); CFLAR(8837); CASP10(843); CASP8(841); |
| | 9; 239; 240; | BMPR2(659); CD28(940); COL4A3(1285); SEPT2(4735); RNF149(284996); |
| | 241; 242; | TLK1(9874); NCKAP1(10787); STK17B(9262); CUL3(8452); HDAC4(9759); |
| | 243; 244; | FANCD2(2177); VHL(7428); PCAF(8850); GNL3(26354); PCNP(57092); |
| | 245; 246; 247; | SMC4L1(10051); FXR1(8087); SEC22L3(9117); NME6(10201); MGC40579 |
| | 248; 249; | (256356); ATR(545); EIF5A2(56648); WHSC1(7468); CD38(952); IL8 |
| | 250; 251; | (3576); EREG(2069); SEPT11(55752); CCNG2(901); FGF5(2250); FGF2(2247); |
| | 10; 252; 25 | TLR2(7097); GAK(2580); APBB2(323); CCNI(10983); LEF1(51176); C |

Table 4-5

| | | |
|---|---|---|
| | 3; 254; 255; | ASP6(839); KIAA1909(153478); ERBB2IP(55914); CCNB1(891); RAD17 |
| | 256; 257; 258; | (5884); F2R(2149); APC(324); TCF7(6932); SEMA5A(9037); UNG2(10309); |
| | 259; 260; | BIRC1(4671); FLJ25680(134187); MCC(4163); ATG12(9140); IRF1(3659); |
| | 261; 262; | PPP2CA(5515); CDC23(8697); CD14(929); CSNK1A1(1452); FBXW11(23291); |
| | 805; 806; 807; | E2F3(1871); TNF(7124); CDKN1A(1026); PARC(23113); VEGF(7422); |
| | 808; 809; | CDC5L(988); PTP4A1(7803); HECA(51696); SASH1(23328); DHX16 |
| | 810; 811; | (8449); IER3(8870); NUDT3(11165); CUL7(9820); TNFRSF21(27242); MCM3 |
| | 812; 813; 814; | (4172); DST(667); ATG5(9474); FRK(2444); SGK(6446); MAP3K5(4217); |
| | 815; 816; | EGFR(1956); GATAD1(57798); RINT1(60561); HBP1(26959); BCAP29 |
| | 817; 2217; | (55973); CASP2(835); CUL1(8454); TRIAD3(54476); PMS2(5395); CYCS |
| | 2218; | (54205); CARD4(10392); INHBA(3624); TMED4(222068); CDK6(1021); RABL5 |
| | | (64792); FASTK(10922); TNFRSF10C(8794); BNIP3L(665); ESCO2 |
| | | (157570); UNC5D(137970); TACC1(6867); UBE2V2(7336); SULF1(23213); |
| | | E2F5(1875); FZD6(8323); MYC(4609); TNFRSF10B(8795); TNFRSF10D |
| | | (8793); TNFRSF10A(8797); SFRP1(6422); DKK4(27121); PAG1(55824); |
| | | TP53INP1(94241); STK3(6788); ANKRD15(23189); CDC37L1(55664); JAK2 |
| | | (3717); UNC13B(10497); RECK(8434); C9orf47(286223); BAG1(573); SEMA4D |
| | | (10507); TNFSF15(9966); TRAF1(7185); PPP6C(5537); UNC5B(219699); |
| | | PTEN(5728); KIF11(3832); BTRC(8945); SUFU(51684); PPP3CB |
| | | (5532); C10orf46(143384); MKI67(4288); BNIP3(664); WEE1(7465); HIPK3 |
| | | (10114); LGALS12(85329); CCND1(595); KIAA1377(57562); BIRC3(330); |
| | | ATM(472); C11orf1(64776); EIF4G2(1982); PTH(5741); HRASLS3(11145); |
| | | MAP3K11(4296); FGF4(2249); FZD4(8322); SESN3(143686); PPP2R1B |
| | | (5519); CCND2(894); CNAP1(9918); IAPP(3375); TEGT(7009); PDE1B |
| | | (5153); APAF1(317); RAN(5901); LRP6(4040); LALBA(3906); FAIM2(23017); |
| | | PAWR(5074); DIP13B(55198); HSMPP8(54737); BRCA2(675); RB1(5925); |
| | | RFP2(10206); BCL2L2(599); CGRRF1(10668); PSEN1(5663); GZMH |
| | | (2999); STRN3(29966); EGLN3(112399); LTBP2(4053); NEK9(91754); AKT1 |
| | | (207); FGF7(2252); KIF23(9493); SLC12A6(9990); BNIP2(663); DAPK2 |
| | | (23604); CSNK1G1(53944); DNASE1(1773); SPN(6693); CYLD(1540); RBL2 |
| | | (5934); MARVELD3(91862); CDT1(81620); PKMYT1(9088); PYCARD |
| | | (29108); FTS(64400); SLC12A4(6560); PAFAH1B1(5048); TNFSF12(8742); |
| | | ALOX15B(247); MAPK7(5598); TRAF4(9618); CROP(51747); NME2 |
| | | (4831); PRR11(55771); TLK2(11011); KPNA2(3838); BIRC5(332); P2RX1(5023); |
| | | NALP1(22861); GAS7(8522); BRCA1(672); ERN1(2081); CABLES1 |
| | | (91768); MAPK4(5596); BCL2(596); TXNL4A(10907); CHAF1A(10036); TNFAIP8L1 |
| | | (126282); DNM2(1785); LDLR(3949); PIK3R2(5296); UBA52(7311); |
| | | CCNE1(898); FOSB(2354); GRLF1(2909); ATF5(22809); PPP2R1A(5518); |
| | | ZNF264(9422); DAPK3(1613); TNFSF14(8740); CDC37(11140); DNASE2 |
| | | (1777); ERF(2077); PPP1R13L(10848); OPA3(80207); CARD8(22900); |
| | | KLK10(5655); PLCB1(23236); POFUT1(23509); MAPRE1(22919); STK4 |
| | | (6789); PARD6B(84612); E2F1(1869); RBL1(5933); PTPRT(11122); ZNFX1 |
| | | (57169); DIDO1(11083); GM632(57473); USP16(10600); SON(6651); |
| | | BTG3(10950); APP(351); BRWD1(54014); SNF1LK(150094); NF2(4771); |
| | | EP300(2033); PPARA(5465); MAPK1(5594); DMC1(11144); PDGFB(5155); |
| | | TAF1(6872); AGTR2(186); BIRC4(331); CD40LG(959); SEPT6(23157); |
| hsa-miR-24 | 11; 263; 264; | DFFB(1677); TNFRSF1B(7133); FBLIM1(54751); NBL1(4681); ZDHHC18 |
| | 265; 266; | (84243); LCK(3932); RBBP4(5928); MACF1(23499); PLK3(1263); CDC14A |
| | 267; 268; 269; | (8556); CYB561D1(284613); RAP1A(5906); LMNA(4000); PEA15(8682); |
| | 270; 271; | UHMK1(127933); FASLG(356); ADORA1(134); TRAF5(7188); TGFB2(7042); |
| | 272; 273; | CIAS1(114548); CDC2L2(985); TNFRSF14(8764); CTNNBIP1(56998); |
| | 274; 275; 276; | FRAP1(2475); MTHFR(4524); CASP9(842); ZBTB17(7709); RCC2(55920); |
| | 277; 278; | TTMB(399474); CLSPN(63967); TESK2(10420); FAF1(11124); BCL10 |
| | 2219; | (8915); NOTCH2(4853); ENSA(2029); CD5L(922); DEDD(9191); ASPM |
| | | (259266); KIF21B(23046); BIRC6(57448); HK2(3099); TCF7L1(83439); KCNIP3 |
| | | (30818); BRRN1(23397); ZAP70(7535); BCL2L11(10018); RIF1(55183); |
| | | TAIP-2(80034); CASP10(843); CASP8(841); DNAJB2(3300); INHA(3623); |
| | | PSMD1(5707); CAPN10(11132); BOK(666); CGREF1(10669); PPM1G |
| | | (5496); CARD12(58484); RTN4(57142); ADD2(119); EDAR(10913); BUB1 |
| | | (699); ANAPC1(64682); IL1A(3552); FLJ10996(54520); IFIH1(64135); PAX3 |
| | | (5077); CTNNB1(1499); LIMD1(8994); MCM2(4171); NEK11(79858); |
| | | SMC4L1(10051); PSMD2(5708); TP73L(8626); OPA1(4976); SRGAP3(9901); |
| | | RAF1(5894); AXUD1(64651); TUSC4(10641); NEK4(6787); STAG1(10274); |
| | | WHSC1(7468); HD(3064); CD38(952); HCAP-G(64151); EREG(2069); |
| | | CCNG2(901); HERC3(8916); NFKB1(4790); LRPAP1(4043); APBB2(3 |

Table 4-6

23); PRKG2(5593); UNC5C(8633); CASP6(839); CASP3(836); CCNB1(891);
F2R(2149); APC(324); TCF7(6932); PHF15(23338); REEP2(51308); NDST1
(3340); BNIP1(662); N4BP3(23138); DAP(1611); UNG2(10309); MCC(4163);
ATG12(9140); SEMA6A(57556); IRF1(3659); C5orf20(140947); PFDN1
(5201); FGF1(2246); CD74(972); DDX41(51428); RIPK1(8737); E2F3(1871);
BTN3A2(11118); TNF(7124); MAPK13(5603); CDKN1A(1026); PARC
(23113); VEGF(7422); IL17(3605); PTP4A1(7803); SASH1(23328):C6orf149
(57128); NEDD9(4739); DHX16(8449); MDC1(9656); DAXX(1616); BAK1
(578); CCND3(896); CUL7(9820); DST(667); SESN1(27244); MAP3K5
(4217); FOXK1(221937); HDAC9(9734); EGFR(1956); FZD9(8326); STAG3
(10734); EMID2(136227); RINT1(60561); BCAP29(55973); CARD4(10392);
NUDCD3(23386); YWHAG(7532); CDK6(1021); MCM7(4176); ZC3HC1
(51530); HIPK2(28996); CDK5(1020); POLR3D(661); RHOBTB2(23221);
BNIP3L(665); ESCO2(157570); BAG4(9530); RIPK2(8767); RHPN1(114822);
KIAA1875(340390); PINX1(54984); BIN3(55909); TNFRSF10B(8795);
ZNF704(619279); REXO1L1(254958); CCNE2(9134); TP53INP1(94241);
MTSS1(9788); JAK2(3717); UNC13B(10497); DAPK1(1612); INVS(27130);
USP20(10868); ABL1(25); ADAMTS13(11093); FLJ36268(401563); CDKN2B
(1030); GAS1(2619); SEMA4D(10507); PTCH(5727); TNFSF15(9966);
TRAF1(7185); TOR2A(27433); CIZ1(25792); TSC1(7248); C10orf9(219771);
SGPL1(8879); UNC5B(219699); LZTS2(84445); SUFU(51684); PDCD11
(22984); CSPG6(9126); IL2RA(3559); LRRC20(55222); DLG5(9231);
FBXW4(6468); SH3PXD2A(9644); MKI67(4288); SPRN(503542); KCNQ1
(3784); STIM1(6786); WEE1(7465); GAS2(2620); MDK(4192); INCENP
(3619); CDC42EP2(10435); DPF2(5977); SIPA1(6494); LRP5(4041); CUL5
(8065); CD3E(916); IGF2(3481); CDKN1C(1028); APBB1(322); WT1(7490);
HRASLS3(11145); BAD(572); MAP3K11(4296); RELA(5970); BRMS1
(25855); PPP1CA(5499); PAK1(5058); CASP4(837); PPP2R1B(5519); IL18
(3606); BCL9L(283149); H2AFX(3014); TNFRSF7(939); PTMS(5763); CDKN1B
(1027); ACVR1B(91); ESPL1(9700); PA2G4(5036); KIAA0152(9761);
TCF1(6927); TNFRSF1A(7132); HDAC7A(51564); LALBA(3906); MCRS1
(10445); FAIM2(23017); C12orf22(81566); DIP13B(55198); ANAPC7(51434);
SCARB1(949); FGF9(2254); TNFRSF19(55504); LATS2(26524); CSNK1A1L
(122011); BCL2L2(599); WDR23(80344); RNF31(55072); SSTR1
(6751); PNN(5411); KIAA0247(9766); PSEN1(5663); VASH1(22846); TRAF3
(7187); LOC400258(400258); ACIN1(22985); DLG7(9787); LTBP2(4053);
NEK9(91754); TGFB3(7043); CHES1(1112); PRIMA1(145270); PPP1R13B
(23368); BUB1B(701); MAPK6(5597); CSK(1445); SCAMP5(192683);
NDN(4692); SLC12A6(9990); BMF(90427); PDCD7(10081); ACSBG1(23205);
AP3S2(10239); LASS3(204219); RAB40C(57799); TSC2(7249); TRAF7
(84231); TNFRSF12A(51330); DNASE1(1773); DNAJA3(9093); PLK1(5347);
PAPD5(64282); RBL2(5934):KATNB1(10300); CTCF(10664); PARD6A
(50855); PSKH1(5681); CHST4(10164); CDT1(81620); NME3(4832); PKMYT1
(9088); XYLT1(64131); ERN2(10595); CMTM4(146223); APPBP1
(8883); TRADD(8717); SLC12A4(6560); RBM35B(80004); TERF2(7014);
Pfs2(51659); PCOLN3(5119); HIC1(3090); ALOX12(239); MAPK7(5598);
TRAF4(9618); NF1(4763); CDC6(990); MAPT(4137); PNPO(55163); CROP
(51747); PPM1D(8493); PRKCA(5578); SLC9A3R1(9368); CDK3(1018);
CARD14(79092); BAIAP2(10458); MNT(4335); P2RX1(5023); NALP1(22861);
GAS7(8522); SPAG5(10615); TIAF1(9220); GAS2L2(246176); CACNB1
(782); BRCA1(672); HDAC5(10014); MPO(4353); CSNK1D(1453); C17orf62
(79415); CIDEA(1149); CABLES1(91768); DAAPRE2(10982); MAPK4
(5596); BCL2(596); TXNL4A(10907); CDC34(997); FZR1(51343); GIPC3(126326);
TNFSF9(8744); ILF3(3609); DNM2(1785); PIK3R2(5296); PSMD8
(5714); MAP3K10(4294); ARHGEF1(9138); APOE(348); SPHK2(56848);
BCL2L12(83596); NALP2(55655); AURKC(6795); RANBP3(8498); RASGRP4
(115727); SIRT2(22933); FLJ13265(79935); TGFB1(7040); ERF(2077);
PPP1R13L(10848); BBC3(27113); LIG1(3978); CGB7(94027); KLK10(5655);
TRIB3(57761); PTPNS1(140885); CDC25B(994); MCM8(84515); ENTPD6
(955); CEP250(11190); MYBL2(4605); STK4(6789); UBE2C(11065);
RTEL1(51750); E2F1(1869); SCAND1(51282); C20orf117(140710); RBL1
(5933); ELMO2(63918); DIDO1(11083); SON(6651); MX1(4599); PDXK(8566);
APP(351); SNF1LK(150094); S100B(6285); BCL2L13(23786); SEPT5
(5413); HIC2(23119); PPIL2(23759); SUSD2(56241); ADORA2A(135); CTB-
1048E9.5(402055); GAS2L1(10634); NF2(4771); LIMK2(3985); MCM

Table 4-7

| | | |
|---|---|---|
| | | 5(4174); EP300(2033); FLJ22349(79879); SEPT3(55964); GTSE1(51512); |
| | | MAPK8IP2(23542); YPEL1(29799); PPM1F(9647); MN1(4330); SSTR3(6753); |
| | | CARD10(29775); PDGFB(5155); TOB2(10766); NHP2L1(4809); FLJ41993 |
| | | (400935); MAPK12(6300); PCTK1(5127); MLLT7(4303); OGT(8473); |
| | | SLC16A2(6567); BIRC4(331); CD40LG(959); SMC1L1(8243); BTK(695); |
| hsa-miR-25 | 12; 279; 280; | RAP1A(5906); FASLG(356); IL19(29949); CIAS1(114548); TESK2(10420); |
| hsa-miR-32 | 281; 282; | FAF1(11124); MCL1(4170); DEDD(9191); BIRC6(57448); PRKCE(5581); |
| | 283; 284; 285; | BCL2L11(10018); RIF1(55183); CASP10(843); ALS2CR19(117583); PIP5K3 |
| | 286; 287; | (200576); COL4A3(1285); CARD12(58484); EIF2AK2(5610); TIA1(7072); |
| | 288; 289; | ANAPC1(64682); MCM6(4175); TLK1(9874); PAX3(5077); SYN2(6854); |
| | 290; 291; 292; | PCAF(8850); CTNNB1(1499); APPL(26060); MCM2(4171); FAIM(55179); |
| | 293; 13; | FXR1(8087); STAG1(10274); TFDP2(7029); ATR(545); HD(3064); EREG |
| | 294; 295; 296; | (2069); FGF2(2247); FLJ20273(54502); CASP6(839); CARD6(84674); MRPS30 |
| | 297; 298; | (10884); GZMA(3001); RAD17(5884); MAN2A1(4124); RAD50(10111); |
| | 299; 300; | PCDHB13(56123); PRKAA1(5562); KIAA1961(96459); IRF1(3659); FGF1 |
| | 301; 302; 303; | (2246); E2F3(1871); FOXO3A(2309); SASH1(23328); BAK1(578); DST(667); |
| | 304; 305; | CCNC(892); LATS1(9113); PDCD2(5134); HDAC9(9734); RINT1(60561); |
| | 818; 819; | HBP1(26959); SOSTDC1(25928); CYCS(54205); CDK6(1021); TACC1 |
| | 820; 821; 822; | (6867); FZD6(8323); TNFRSF10B(8795); ADRA1A(148); CLU(1191); CCNE2 |
| | 823; 824; | (9134); RAD21(5885); TNFRSF11B(4982); TRAF1(7185); TSC1(7248); |
| | 825; 826; | PTEN(5728); BTRC(8945); PARD3(56288); DLG5(9231); C10orf46(143384); |
| | 827; 828; 829; | IFITM1(8519); STIM1(6786); HIPK3(10114); BIRC3(330); CUL5(8065); |
| | 830; 831; | CDKN1C(1028); DKK3(27122); FZD4(8322); SESN3(143686); ZW10(9183); |
| | 832; 833; | DYRK2(8445); MDM2(4193); NUDT4(11163); GAS2L3(283431); FZD10 |
| | 834; 835; 836; | (11211); CD69(969); BCAT1(586); KRAS(3845); STAT2(6773); BTG1(694); |
| | 837; 838; | DIP13B(55198); TMEM119(338773); SCARB1(949); HSMPP8(54737); |
| | 839; 840; | ING1(3621); CUL4A(8451); LATS2(26524); CGRRF1(10668); TRAF3(7187); |
| | 841; 842; 843; | STRN3(29966); LTBP2(4053); NEK9(91754); MOAP1(64112); PPPIR13B |
| | 844; 845; | (23368); SPN(6693); PAPD5(64282); RBL2(5934); NOL3(8996); CTCF |
| | 846; 847; | (10664); CDT1(81620); EXOSC6(118460); PHLPPL(23035); PAFAH1B1(5048); |
| | 848; 849; 850; | NLK(51701); NF1(4763); CDC6(990); PPM1D(8493); MAP2K6(5608); |
| | 851; | CETN1(1068); DCC(1630); ESCO1(114799); BCL2(596); FZR1(51343); TNFSF9 |
| | | (8744); AXL(558); APOE(348); ZNF530(348327); MCM8(84515); CTNNBL1 |
| | | (56259); PARD6B(84612); AURKA(6790); CTCFL(140690); DIDO1 |
| | | (11083); APP(351); SNF1LK(150094); CDC45L(8318); NF2(4771); PGEA1 |
| | | (25776); TOB2(10766); PCTK1(5127); TAF1(6872); BIRC4(331); PDCD8 |
| | | (9131); |
| hsa-miR-26a | 14; 306; 307; | DFFB(1677); KHDRBS1(10657); RBBP4(5928); MACF1(23499); RAP1A |
| hsa-miR-26b | 308; 309; | (5906); UHMK1(127933); IL24(11009); PFKFB2(5208); CCNL2(81669); CLSPN |
| | 310; 311; 312; | (63967); FAF1(11124); SASS6(163786); MCL1(4170); ASPM(259266); |
| | 313; 314; | BIRC6(57448); TAIP-2(80034); ZAK(51776); CFLAR(8837); CASP10(843); |
| | 315; 316; | CASP8(841); COL4A3(1285); SEPT10(151011); BUB1(699); ANAPC1 |
| | 317; 318; 319; | (64682); MCM6(4175); TLK1(9874); STB17B(9262); BARD1(580); USP37 |
| | 320; 15; | (57695); PAX3(5077); CUL3(8452); HDAC4(9759); PDCD6IP(10015); TDGF1 |
| | 321; 322; 323; | (6997); FXR1(8087); B3GNT5(84002); THRB(7068); NEK4(6787); GSK3B |
| | 324; 22 | (2932); TNFSF10(8743); PI4R2B(55300); ANAPC4(29945); EREG(2069); |
| | 20; | CCNG2(901); PLK4(10733); TLR2(7097); GABRA4(2557); CCNI(10983); |
| | | UNC5C(8633); CXXC4(80319); LEF1(51176); SLC7A11(23657); NEK1 |
| | | (4750); SKP2(6502); NIPBL(25836); ERBB2IP(55914); APC(324); LOC153222 |
| | | (153222); PRLR(5618); PRKAA1(5562); CD74(972); MAPK13(5603); |
| | | PTP4A1(7803); TTK(7272); BAK1(578); SUPT3H(8464); DST(667); ASCC3 |
| | | (10973); PDCD2(5134); AHR(196); HDAC9(9734); STK17A(9263); CUL1 |
| | | (8454); SFRP4(6424); INHBA(3624); CDK6(1021); BAG4(9530); UBE2V2 |
| | | (7336); SULF1(23213); FZD6(8323); WISP1(8840); TNFRSF10B(8795); TNFRSF10D |
| | | (8793); TP53INP1(94241); CDC37L1(55664); DAPK1(1612); |
| | | CKS2(1164); SEMA4D(10507); C10orf9(219771); REEP3(221035); PTEN |
| | | (5728); CSPG6(9126); DOCK1(1793); CUL2(8453); PPP3CB(5532); DLG5 |
| | | (9231); FRAT2(23401); TIAL1(7073); WEE1(7465); PHCA(55331); BIRC3 |
| | | (330); ATM(472); EIF4G2(1982); MTL5(9633); PAK1(5058); CHORDC1(26973); |
| | | SESN3(143686); CASP4(837); PPP2R1B(5519); CCND2(894); ETNK1 |
| | | (55500); KLHDC5(57542); CRADD(8738); APAF1(317); BCAT1(586); C12orf22 |
| | | (81566); PAWR(5074); BTG1(694); ANAPC7(51434); DIABLO(56616); |
| | | CDK2AP1(8099); CHFR(55743); FGF9(2254); TNFRSF19(55504); CDK8 |
| | | (1024); BRCA2(675); RB1(5925); CDC16(8881); CSNK1A1L(122011); |
| | | FOX01A(2308); TXNDC(81542); DAD1(1603); CDKL1(8814); CHES1(11 |

Table 4-8

| | | |
|---|---|---|
| | | 12); MAPK6(5597); AVEN(57099); BNIP2(663); CSNK1G1(53944); CYLD |
| | | (1540); AXIN1(8312); CDC6(990); KPNA2(3838); CARD14(79092); AURKB |
| | | (9212); ERN1(2081); HELZ(9931); MAPRE2(10982); MALT1(10892); ESCO1 |
| | | (114799); CHAF1A(10036); CCNE1(898); LOC390980(390980); MCM8 |
| | | (84515); PLCB1(23236); CSNK2A1(1457); PCNA(5111); AURKA(6790); |
| | | DIDO1(11083); SON(6651); BTG3(10950); BRWD1(54014); SNF1LK(150094); |
| | | SEPT3(55964); GTSE1(51512); MGC70863(284942); BID(637); DMC1 |
| | | (11144); TAF1(6872); RP6-213H19.1(51765); |
| hsa-miR-27b | 16; 325; 326; | DFFB(1677); C1orf144(26099); PAX7(5081); NBL1(4681); CDC42(998); SESN2 |
| | 327; 328; | (83667); KHDRBS1(10657); MACF1(23499); ST6GALNAC3(256435); |
| | 329; 330; 331; | CDC14A(8556); VANGL1(81839); BNIPL(149428); S100A7L1(338324); |
| | 332; 333; | DAP3(7818); RGS2(5997); ADORA1(134); IL24(11009); SPHAR(10638); |
| | 334; 335; | CLAS1(114548); CCNL2(81669); TNFRSF9(3604); DFFA(1676); FRAP1(2475); |
| | 336; 337; 338; | CASP9(842); RCC2(55920); MUTYH(4595); TESK2(10420); SASS6 |
| | 339; 340; | (163786); NOTCH2(4853); MCL1(4170); SHE(126669); LPGAT1(9926); PPP1CB |
| | 341; 342; | (5500); BIRC6(57448); HK2(3099); BRRN1(23397); BCL2L11(10018); |
| | 343; 344; 345; | CCNT2(905); FZD7(8324); CD28(940); YWHAQ(10971); E2F6(1876); RTN4 |
| | 346; 347; | (57142); CCT4(10575); TGFA(7039); EDAR(10913); SEPT10(151011); |
| | 348; 349; | BUB1(699); ANAPC1(64682); BIN1(274); MCM6(4175); TLK1(9874); STAT1 |
| | 350; 351; 352; | (6772); STK17B(9262); CLK1(1195); PAX3(5077); SLC6A1(6529); TDGF1 |
| | 353; 354; | (6997); RBM5(10181); APPL(26060); SEC22L2(26984); NEK11(79858); |
| | 355; 356; | ACPP(55); ARMC8(25852); FXR1(8087); KNG1(3827); TP73L(8626); OPA1 |
| | 852; 2221; | (4976); CIDEC(63924); AXUD1(64651); NEK4(6787); FHIT(2272); STAG1 |
| | | (10274); MUC4(4585); HD(3064); EREG(2069); CCNG2(901); FGF5(2250); |
| | | NFKB1(4790); GAB1(2549); GAK(2580); APBB2(323); CCNI(10983); |
| | | DKK2(27123); CASP6(839); ARSJ(79642); SLC7A11(23657); SFRP2(6423); |
| | | NEK1(4750); VEGFC(7424); NIPBL(25836); APC(324); TCF7(6932); PRLR |
| | | (5618); PLK2(10769); MCC(4163); SEMA6A(57556); CDC25C(995); FGF1 |
| | | (2246); RIPK1(8737); CSNK2B(1460); MAPK13(5603); CDKN1A(1026); |
| | | IL17(3605); BAG2(9532); FOXO3A(2309); TNFAIP3(7128); SASH1(23328); |
| | | QK1(9444); NEDD9(4739); DHX16(8449); MDC1(9656); IER3(8870); |
| | | BAK1(578); CUL7(9820); SUPT3H(8464); DST(667); CCNC(892); MAP3K5 |
| | | (4217); PLAGL1(5325); KATNA1(11104); LATS1(9113); HDAC9(9734); EGFR |
| | | (1956); HBP1(26959); BCAP29(55973); CUL1(8454); CARD4(10392); |
| | | ELMO1(9844); IGFBP3(3486); PMS2L2(5380); CDK6(1021); BNIP3L(665); |
| | | PTK2B(2185); UNC5D(137970); FZD6(8323); WISP1(8840); GML(2765); |
| | | BIN3(55909); TNFRSF10D(8793); CLU(1191); SFRP1(6422); TP53INP1 |
| | | (94241); NTRK2(4915); ABL1(25); FANCG(2189); GAS1(2619); PTCH |
| | | (5727); TNFSF15(9966); TNFSF8(944); TRAF1(7185); TSC1(7248); ZMYND11 |
| | | (10771); C10orf9(219771); SIRT1(23411); SGPL1(8879); FAS(355); PDCD11 |
| | | (22984); IL2RA(3559); PARD3(56288); CUL2(8453); ZWINT(11130); |
| | | DLG5(9231); GPAM(57678); DCLRE1A(9937); MKI67(4288); BNIP3(664); |
| | | WEE1(7465); EXT2(2132); LGALS12(85329); VEGFB(7423); DPF2(5977); |
| | | SSSCA1(10534); LRP5(4041); FADD(8772); TNFRSF19L(84957); PANX1 |
| | | (24145); CUL5(8065); ATM(472); LOH11CR2A(4013); WT1(7490); PPP1CA |
| | | (5499); MTL5(9633); PPP2R1B(5519); ZW10(9183); TNFRSF7(939); |
| | | PTPN6(5777); TEGT(7009); LETMD1(25875); APAF1(317); LRP6(4040); |
| | | KRAS(3845); WIF1(11197); DUSP6(1848); PPP1CC(5501); DIABLO(56616); |
| | | RFP2(10206); CDC16(8881); FOXO1A(2308); NEK3(4752); ZIC5(85416); |
| | | TXNDC(81542); CGRRF1(10668); PSEN1(5663); GPR65(8477); TRAF3 |
| | | (7187); CDKL1(8814); LTBP2(4053); NEK9(91754); CHES1(1112); JAG2 |
| | | (3714); FGF7(2252); MAPK6(5597); CSK(1445); SLC12A6(9990); BCL2L10 |
| | | (10017); DAPK2(23604); CSNK1G1(53944); BCL2A1(597); TSC2(7249); |
| | | TRAF7(84231); KREMEN2(79412); CTCF(10664); CDK10(8558); PKMYT1 |
| | | (9088); GSPT1(2935); ERN2(10595); SLC12A4(6560); SMPD3(55512); |
| | | PHLPPL(23035); HIC1(3090); TNFSF12(8742); MAP2K4(6416); MAPK7 |
| | | (5598); KSR1(8844); TRAF4(9618); NF1(4763); NGFR(4804); NME1(4830); |
| | | TLK2(11011); GPS1(2873); MNT(4335); P2RX1(5023); UNQ5783(388325); |
| | | TP53(7157); SPAG5(10615); FLJ34922(91607); BRCA1(672); TMEM101 |
| | | (84336); HDAC5(10014); PHB(5245); SEPT4(5414); CSNK1D(1453); CCDC5 |
| | | (115106); DCC(1630); PHLPP(23239); ESCO1(114799); HDHD2(84064); |
| | | TXNL1(9352); BCL3(602); APOE(348); FOSB(2354); FOXA3(3171); |
| | | BAX(581); PPP2R1A(5518); MATK(4145); DPF1(8193); BBC3(27113); PARD6B |
| | | (84612); E2F1(1869); UBE2V1(7335); CTCFL(140690); CABLES2 |
| | | (81928); DIDO1(11083); SON(6651); HEMK2(29104); SNF1LK(150094); I |

Table 4-9

| | | |
|---|---|---|
| | | TGB2(3689); BCL2L13(23786); KREMEN1(83999); MAPK8IP2(23542); |
| | | BID(637); MAPK1(5594); MN1(4330); CARD10(29775); MAPK12(6300); |
| | | CCNB3(85417); TAF1(6872); NGFRAP1(27018); CD40LG(959); PRKX(5613); |
| | | CUL4B(8450); ARHGEF6(9459); CETN2(1069); HCFC1(3054); PRKY |
| | | (5616); |
| hsa-miR-28 | 17; 357; 358; | PRKCZ(5590); TP73(7161); DFFB(1677); NUDC(10726); SESN2(83667); |
| | 359; 360; | LCK(3932); RBBP4(5928); MACF1(23499); PLK3(1263); GADD45A(1647); |
| | 361; 362; 363; | SH3GLB1(51100); CDC14A(8556); RAP1A(5906); BCL9(607); FASLG(356); |
| | 364; 365; | QSCN6(5768); NAV1(89796); C1orf95(375057); CIAS1(114548); TNFRSF14 |
| | 366; 367; | (8764); CTNNBIP1(56998); DFFA(1676); MTHFR(4524); ZBTB17 |
| | 368; 853; 854; | (7709); RCC2(55920); KIAA0319L(79932); MUTYH(4595); FAF1(11124); NRAS |
| | 855; | (4893); NOTCH2(4853); GON4L(54856); TNFSF18(8995); ASPM(259266); |
| | | TP53BP2(7159); PRKCE(5581); GEN1(56287); HTRA2(27429); HK2 |
| | | (3099); INPP4A(3631); PROC(5624); ZAK(51776); CD28(940); ALS2CR19 |
| | | (117583); RHBDD1(84236); E2F6(1876); CGREF1(10669); RTN4(57142); |
| | | TMEM127(55654); SEPT10(151011); ANAPC1(64682); TLK1(9874); HDAC4 |
| | | (9759); PDCD6IP(10015); MLH1(4292); CTNNB1(1499); NEK11(79858); |
| | | SMC4L1(10051); KNG1(3827); TP73L(8626); OPA1(4976); TADA3L |
| | | (10474); CIDEC(63924); THRB(7068); RASSF1(11186); IL17RD(54756); |
| | | UBE1C(9039); PIK3CB(5291); ATR(545); HD(3064); CD38(952); SEPT11 |
| | | (55752); CCNG2(901); GAK(2580); APBB2(323); PRKG2(5593); UNC5C(8633); |
| | | LEF1(51176); MAD2L1(4085); NIPBL(25836); ERBB2IP(55914); F2R |
| | | (2149); APC(324); TCF7(6932); G3BP(10146); BNIP1(662); UNC5A(90249); |
| | | N4BP3(23138); DAP(1611); PRLR(5618); BIRC1(4671); MCC(4163); PFDN1 |
| | | (5201); FGF1(2246); DUSP1(1843); ADAMTS2(9509); RIPK1(8737); |
| | | LY86(9450); E2F3(1871); ZNF192(7745); TCF19(6941); AIF1(199); CSNK2B |
| | | (1460); CDKN1A(1026); PARC(23113); CDC5L(988); PTP4A1(7803); |
| | | TTK(7272); FOXO3A(2309); HECA(51696); SASH1(23328); DHX16(8449); |
| | | DAXX(1616); IHPK3(117283); CCND3(896); CUL7(9820); DST(667); SESN1 |
| | | (27244); FRK(2444); LATS1(9113); AGPAT4(56895); PDCD2(5134); EGFR |
| | | (1956); FZD1(8321); STAG3(10734); BCAP29(55973); CASP2(835); |
| | | MGC11257(84310); MAD1L1(8379); TRIAD3(54476); CARD4(10392); ELMO1 |
| | | (9844); LOC401357(401357); YWHAG(7532); MCM7(4176); KIAA0773 |
| | | (9715); FASTK(10922); PTK2B(2185); ESCO2(157570); UNC5D(137970); |
| | | UBE2V2(7336); RIPK2(8767); WISP1(8840); LRRC14(9684); BIN3(55909); |
| | | TNFRSF10D(8793); DUSP4(1846); MOS(4342); MTSS1(9788); ZNF |
| | | 406(57623); JAK2(3717); NTRK2(4915); PAPPA(5069); ABL1(25); TRAF2 |
| | | (7186); NDOR1(27158); CDKN2B(1030); BAG1(573); GAS1(2619); SEMA4D |
| | | (10507); PTCH(5727); TNFSF15(9966); TNFSF8(944); TRAF1(7185); SGPL1 |
| | | (8879); UNC5B(219699); PTEN(5728); LZTS2(84445); BTRC(8945); |
| | | NOLC1(9221); SUFU(51684); TCF7L2(6934); GPR26(2849); DOCK1(1793); |
| | | PARD3(56288); FZD8(8325); DLG5(9231); HPS1(3257); FBXW4(6468); |
| | | TIAL1(7073); MKI67(4288); BNIP3(664); HIPK3(10114); F2(2147); INCENP |
| | | (3619); SSSCA1(10534); SIPA1(6494); CCND1(595); FADD(8772); CUL5 |
| | | (8065); CD3G(917); LOH11CR2A(4013); IGF2(3481); EIF4G2(1982); |
| | | DKK3(27122); PTH(5741); HRASLS3(11145); BAD(572); RELA(5970); MTL5 |
| | | (9633); FGF3(2248); SESN3(143686); BTG4(54766); BCL9L(283149); |
| | | CCND2(894); CNAP1(9918); PTMS(5763); TEGT(7009); ESPL1(9700); PDE1B |
| | | (5153); CDK2(1017); ZNFN1A4(64375); APAF1(317); GAS2L3(283431); |
| | | KNTC1(9735); FZD10(11211); HDAC7A(51564); ZNF641(121274); |
| | | MCRS1(10445); FMNL3(91010); FAIM2(23017); DDIT3(1649); PAWR(5074); |
| | | BTG1(694); DIP13B(55198); CIT(11113); SCARB1(949); HSMPP8(54737); |
| | | ATP11A(23250); FOXO1A(2308); BCL2L2(599); VASH1(22846); GPR65 |
| | | (8477); ACIN1(22985); CIDEB(27141); PGF(5228); NEK9(91754); CHES1 |
| | | (1112); AKT1(207); JAG2(3714); MAPK6(5597); SNX22(79856); CSK |
| | | (1445); NDN(4692); BMF(90427); CSNK1G1(53944); TSC2(7249); TRAF7 |
| | | (84231); CCNF(899); TNFRSF12A(51330); THOC6(79228); PLK1(5347); CARD15 |
| | | (64127); CYLD(1540); HSPC065(29070); E2F4(1874); KIAA1576 |
| | | (57687); LOC348180(348180); CDK10(8558); NME3(4832); FAM100A(124402); |
| | | ERN2(10595); CSNK2A2(1459); SMPD3(55512); TXNL4B(54957); PCOLN3 |
| | | (5119); PAFAH1B1(5048); ALOX12(239); TNFSF12(8742); MAPK7 |
| | | (5598); PNPO(55163); NGFR(4804); SPHK1(8877); CARD14(79092); METT10D |
| | | (79066); P2RX1(5023); TP53(7157); SPAG5(10615); BRCA1(672); |
| | | HDAC5(10014); SCRN2(90507); PTRH2(51651); ERN1(2081); RECQL5 |
| | | (9400); C18orf24(220134); MAPK4(5596); MALT1(10892); PHLPP(23239); |

Table 4-10

| | | |
|---|---|---|
| | | TXNL1(9352); GADD45B(4616); FZR1(51343); CHAF1A(10036); UHRF1 |
| | | (29128); APLP1(333); DMRTC2(63946); BCAM(4059); FOSB(2354); BCL2L12 |
| | | (83596); NALP2(55655); FLJ45445(399844); MATK(4145); PLAC2(257000); |
| | | DNASE2(1777); DPF1(8193); SIRT2(22933); DEDD2(162989); TRIB3 |
| | | (57761); MYBL2(4605); STK4(6789); RBL1(5933); CABLES2(81928); SON |
| | | (6651); MX1(4599); AGPAT3(56894); APP(351); SNF1LK(150094); BCL2L13 |
| | | (23786); SMARCB1(6598); ADORA2A(135); GAS2L1(10634); NF2 |
| | | (4771); MTP18(51537); YWHAH(7533); MCM5(4174); SEPT3(55964); BIK |
| | | (638); GTSE1(51512); FLJ21125(79680); MAPK1(5594); PPM1F(9647); CHEK2 |
| | | (11200); SSTR3(6753); CARD10(29775); SMC1L2(27127); LOC150383 |
| | | (150383); SHOX(6473); IKBKG(8517); DKC1(1736); PPP2R3B(28227); |
| | | IQSEC2(23096); SMC1L1(8243); SEPT6(23157); CUL4B(8450); MTCP1 |
| | | (4515); |
| hsa-miR-29a | 18; 369; 370; | DFFB(1677); CDC42(998); RCC1(1104); MACF1(23499); SH3GLB1(51100); |
| hsa-miR-29c | 371; 372; | CDC7(8317); RAP1A(5906); VANGL1(81839); NAV1(89796); IPO9(55705); |
| | 373; 374; 375; | IL24(11009); G0S2(50486); TRAF5(7188); TGFB2(7042); CCNL2(81669); |
| | 376; 377; | TNFRSF9(3604); CTNNBIP1(56998); CASP9(842); RCC2(55920); YARS |
| | 378; 379; | (8565); MUTYH(4595); TESK2(10420); ITGB3BP(23421); NRAS(4893); |
| | 380; 381; 382; | NOTCH2(4853); MCL1(4170); DEDD(9191); RNASEL(6041); ASPM(259266); |
| | 383; 384; | MAPRE3(22924); BIRC6(57448); PRKCE(5581); MSH2(4436); KCNIP3 |
| | 385; 386; | (30818); ZAK(51776); CASP8(841); FZD7(8324); LOC92691(92691); |
| | 387; 388; 389; | INHA(3623); COL4A3(1285); CGREF1(10669); RTN4(57142); CCT4(10575); |
| | 390; 391; | MOBK1B(55233); EDAR(10913); ANAPC1(64682); BIN1(274); ERCC3 |
| | 392; 393; | (2071); WDR33(55339); STAT1(6772); FZD5(7855); COL4A4(1286); HDAC4 |
| | 394; 19; 395; | (9759); FANCD2(2177); DLEC1(9940); CTNNB1(1499); APPL(26060); |
| | 396; 397; | OPA1(4976); CIDEC(63924); FHIT(2272); UBE1C(9039); GSK3B(2932); SIAH2 |
| | 398; 399; 400; | (6478); WHSC1(7468); HD(3064); CD38(952); ANAPC4(29945); EREG |
| | 856; 2222; | (2069); SEPT11(55752); NFKB1(4790); PLK4(10733); TLR2(7097); ANAPC10 |
| | | (10393); CARD6(84674); ERBB2IP(55914); APC(324); CSNK1G3 |
| | | (1456); DAP(1611); GDNF(2668); BIRC1(4671); MCC(4163); IRF1(3659); PPP2CA |
| | | (5515); CDC23(8697); CD14(929); NR3C1(2908); ADAMTS2(9509); |
| | | CSNK2B(1460); PARC(23113); SLC22A7(10864); VEGF(7422); PTP4A1 |
| | | (7803); FOXO3A(2309); TNFAIP3(7128); SASH1(23328); NEDD9(4739); |
| | | DHX16(8449); BAK1(578); CUL7(9820); TNFRSF21(27242); MCM3(4172); |
| | | CCNC(892); SESN1(27244); FRK(2444); SGK(6446); PLAGL1(5325); LATS1 |
| | | (9113); PDCD2(5134); AHR(196); HDAC9(9734); FZD9(8326); ELN(2006); |
| | | HBP1(26959); CASP2(835); SNX8(29886); CYCS(54205); ELMO1(9844); |
| | | IGFBP3(3486); CDK6(1021); MCM7(4176); FASTK(10922); POLR3D |
| | | (661); BAG4(9530); TACC1(6867); PI15(51050); MYC(4609); WISP1(8840); |
| | | PINX1(54984); TNFRSF10B(8795); TNFRSF10A(8797); SFRP1(6422); |
| | | MOS(4342); CCNE2(9134); TP53INP1(94241); UNC13B(10497); DAPK1 |
| | | (1612); CDK9(1025); ABL1(25); COL5A1(1289); IFNW1(3467); IFNA2(3440); |
| | | CDKN2A(1029); CDKN2B(1030); SEMA4D(10507); TNFSF8(944); TRAF1 |
| | | (7185); TSC1(7248); ANAPC2(29882); SIRT1(23411); CXXC6(80312); |
| | | UNC5B(219699); PTEN(5728); FAS(355); KIF11(3832); SUFU(51684); |
| | | PDCD11(22984); C10orf67(256815); C10orf55(414236); FRAT2(23401); SFRP5 |
| | | (6425); SH3PXD2A(9644); DCLRE1A(9937); C10orf46(143384); TIAL1 |
| | | (7073); MKI67(4288); IFITM1(8519); WEE1(7465); EXT2(2132); F2(2147); |
| | | SIPA1(6494); RPS6KB2(6199); LRP5(4041); FADD(8772); BIRC2(329); |
| | | CUL5(8065); ATM(472); LOH11CR2A(4013); HRAS(3265); IGF2(3481); |
| | | DKK3(27122); CDCA5(113130); BRMS1(25855); FZD4(8322); CASP4(837); |
| | | PPP2R1B(5519); ZW10(9183); BCL9L(283149); H2AFX(3014); CCND2 |
| | | (894); LTBR(4055); CNAP1(9918); ESPL1(9700); PDE1B(5153); CDK2(1017); |
| | | MDM2(4193); CCT2(10576); CRADD(8738); GAS2L3(283431); TDG |
| | | (6996); KNTC1(9735); FZD10(11211); TNFRSF1A(7132); CLEC7A(64581); |
| | | LRP6(4040); HDAC7A(51564); C12orf22(81566); CBX5(23468); DUSP6 |
| | | (1848); HRK(8739); ANAPC5(51433); DIABLO(56616); CHFR(55743); TNFRSF19 |
| | | (55504); RB1(5925); CUL4A(8451); LIG4(3981); TXNDC(81542); |
| | | PSEN1(5663); VASH1(22846); TRAF3(7187); CIDEB(27141); STRN3(29966); |
| | | RAB15(376267); DIO2(1734); CHES1(1112); BCL11B(64919); PPP1R13B |
| | | (23368); TMEM62(80021); MAPK6(5597); KIAA1024(23251); IGF1R |
| | | (3480); NDN(4692); BMF(90427); DAPK2(23604); CSNK1G1(53944); RAB40C |
| | | (57799); TSC2(7249); CCNF(899); KREMEN2(79412); DNASE1(1773); |
| | | KATNB1(10300); NDRG4(65009); HAS3(3038); ZNF276(92822); NME3 |
| | | (4832); ERN2(10595); SIAH1(6477); SLC12A4(6560); SMPD3(55512); HI |

Table 4-11

| | | |
|---|---|---|
| | | C1(3090); PAFAH1B1(5048); GSG2(83903); ALOX15B(247); MAPK7(5598); |
| | | TRAF4(9618); NF1(4763); LIG3(3980); CDC6(990); CROP(51747); NME1 |
| | | (4830); PPM1D(8493); SPHK1(8877); CARD14(79092); P2RX1(5023); |
| | | GAS7(8522); GAS2L2(246176); BRCA1(672); HDAC5(10014); MPO(4353); |
| | | SEPT4(5414); ERN1(2081); MAFG(4097); CSNK1D(1453); CIDEA(1149); |
| | | MAPRE2(10982); MAPK4(5596); ESCO1(114799); BCL2(596); CSNK1G2 |
| | | (1455); DOT1L(84444); FZR1(51343); CHAF1A(10036); UHRF1(29128); |
| | | TNFSF9(8744); PIK3R2(5296); AXL(558); ARHGEF1(9138); APOE(348); |
| | | BAX(581); NALP2(55655); COL5A3(50509); ZNF443(10224); DPF1(8193); |
| | | TGFB1(7040); ERF(2077); PPP1R13L(10848); BBC3(27113); LIG1(3978); |
| | | LENG9(94059); TRIB3(57761); MCM8(84515); MAPRE1(22919); MYBL2 |
| | | (4605); CD40(958); E2F1(1869); RBL1(5933); ELMO2(63916); UBE2V1 |
| | | (7335); CTCFL(140690); CABLES2(81928); DIDO1(11083); SON(6651); |
| | | MX1(4599); SNF1LK(150094); CDC45L(8318); SEPT5(5413); KREMEN1 |
| | | (83999); GAS2L1(10634); NF2(4771); PGEA1(25776); BID(637); FLJ21125 |
| | | (79680); ARVCF(421); C1QTNF6(114904); SSTR3(6753); CARD10(29775); |
| | | PDGFB(5155); TOB2(10766); POLR3H(171568); MAPK12(6300); HDAC6 |
| | | (10013); STAG2(10735); DCX(1641); SEPT6(23157); CUL4B(8450); HCFC1 |
| | | (3054); |
| hsa-miR-30b | 20; 401; 402; | TP73(7161); S100PBP(64766); PRKAA2(5563); SH3GLB1(51100); CDC7 |
| hsa-miR-30c | 403; 404; | (8317); PTGFRN(5738); BCL9(607); UHMK1(127933); RGS2(5997); CIAS1 |
| | 405; 406; 407; | (114548); BCL10(8915); NOTCH2(4853); GLRX2(51022); ASPM(259266); |
| | 408; 409; | ZBTB41(360023); TP53BP2(7159); RHOB(388); BIRC6(57448); MAL(4118); |
| | 410; 411; | PDCL3(79031); BCL2L11(10018); TMEM87B(84910); RIF1(55183); SCN2A2 |
| | 412; 413; 414; | (6326); TIA1(7072); EDAR(10913); ANAPC1(64682); IL1A(3552); IFIH1 |
| | 415; 416; | (64135); FRZB(2487); NCKAP1(10787); STAT1(6772); STK17B(9262); |
| | 417; 418; | BARD1(580); USP37(57695); CUL3(8452); TRNT1(51095); RBM5(10181); |
| | 419; 420; 421; | ACPP(55); RNF7(9616); B3GNT5(84002); SENP2(59343); TP73L(8626); |
| | 422; 423; | CELSR3(1951); NEK4(6787); UBE1C(9039); PROK2(60675); GCET2 |
| | 424; 425; | (257144); CD80(941); STAG1(10274); HD(3064); HCAP-G(64151); FGF5(2250); |
| | 426; 427; 428; | NFKB1(4790); TLR2(7097); STOX2(56977); APBB2(323); CCNI(10983); |
| | 429; 430; | UNC5C(8633); OTUD4(54726); NEK1(4750); VEGFC(7424); CASP3 |
| | 431; 432; | (836); SKP2(6502); NIPBL(25836); TCF7(6932); SLC36A1(206358); PRLR |
| | 433; 434; 435; | (5618); DKFZP781I1119(166968); ANKRA2(57763); MCC(4163); ATG12 |
| | 436; 437; | (9140); SEMA6A(57556); PPP2CA(5515); CSNK1A1(1452); FBXW11(23291); |
| | 438; 439; | E2F3(1871); RUNX2(860); IL17(3605); PTP4A1(7803); LIN28B(389421); |
| | 440; 441; 442; | DHX16(8449); IHPK3(117283); SUPT3H(8464); DST(667); ATG5(9474); |
| | 443; 21; | FRK(2444); BCLAF1(9774); MAP3K5(4217); AHR(196); HDAC9(9734); |
| | 444; 445; 446; | SEPT7(989); PHTF2(57157); BCAP29(55973); CYCS(54205); INHBA(3624); |
| | 447; 448; | CDK6(1021); MCM7(4176); BRAF(673); BNIP3L(665); ESCO2(157570); |
| | 449; 450; | FZD3(7976); UNC5D(137970); BAG4(9530); TACC1(6867); UBE2V2(7336); |
| | 451; 452; 453; | FZD6(8323); EBAG9(9166); WISP1(8840); LZTS1(11178); TNFRSF10B |
| | 454; 455; | (8795); TNFRSF10D(8793); TRIM35(23087); CCNE2(9134); TP53INP1 |
| | 857; 858; | (94241); MTSS1(9788); CDC37L1(55664); JAK2(3717); UNC13B(10497); |
| | 859; 860; 861; | RECK(8434); ANKRD20A2(441430); ANKRD20A1(84210); DAPK1(1612); |
| | 2223; 2224; | ABL1(25); TAF1L(138474); ANKRD20A3(441425); KIAA0367(23273); |
| | | TNFSF8(944); TSC1(7248); C10orf9(219771); REEP3(221035); SIRT1(23411); |
| | | KIF11(3832); BTRC(8945); DOCK1(1793); IL2RA(3559); CUL2(8453); |
| | | RP11-142I17.1(26095); PLEKHK1(219790); DLG5(9231); DCLRE1A |
| | | (9937); C10orf46(143384); HIPK3(10114); INCENP(3619); BIRC3(330); BIRC2 |
| | | (329); CUL5(8065); ATM(472); PAFAH1B2(5049); LOH11CR2A(4013); |
| | | SLC36A4(120103); CASP4(837); CASP5(838); BCL2L14(79370); APAF1 |
| | | (317); GAS2L3(283431); KNTC1(9735); FZD10(11211); RAN(5901); LRP6 |
| | | (4040); KRAS(3845); PAWR(5074); BTG1(694); PCTK2(5128); DIP13B(55198); |
| | | PPP1CC(5501); DIABLO(56616); RIMBP2(23504); HSMPP8(54737); |
| | | TNFRSF19(55504); RFP2(10206); ING1(3621); TFDP1(7027); CDC16(8881); |
| | | LATS2(26524); LIG4(3981); PNN(5411); VASH1(22846); GPR65(8477); |
| | | TRAF3(7187); JAG2(3714); MKRN3(7681); MAPK6(5597); CCNB2(9133); |
| | | FAM81A(145773); AVEN(57099); CCPG1(9236); NEDD4(4734); CSNK1G1 |
| | | (53944); FAHD1(81889); CCNF(899); DNAJA3(9093); TNRC6A(27327); |
| | | NFAT5(10725); GSPT1(2935); SIAH1(6477); FTS(64400); GSG2(83903); |
| | | ALOX12(239); NLK(51701); NF1(4763); LIG3(3980); CARD14(79092); |
| | | MNT(4335); NALP1(22861); BECN1(8678); HDAC5(10014); SOCS3(9021); |
| | | USP14(9097); WDR7(23335); MALT1(10892); PHLPP(23239); ESCO1 |
| | | (114799); TXNL1(9352); TNFSF9(8744); PIK3R2(5296); AXL(558); TNFS |

Table 4-12

| | | |
|---|---|---|
| | | F14(8740); DPF1(8193); KLK10(5655); TRIB3(57761); STK35(140901); C20orf91 |
| | | (284800); MYBL2(4605); STK4(6789); CSE1L(1434); PCMTD2(55251); |
| | | BCL2L1(598); RBM12(10137); CTCFL(140690); SYCP2(10388); USP16 |
| | | (10600); SON(6651); SLC5A3(6526); MX1(4599); BRWD1(54014); SEPT3 |
| | | (55964); BID(637); MAPK1(5594); SMC1L2(27127); STS(412); CNKSR2 |
| | | (22866); TAF1(6872); STAG2(10735); DKC1(1736); SEPT6(23157); CUL4B |
| | | (8450); ARHGEF6(9459); MTCP1(4515); |
| hsa-miR-93 | 22; 456; 457; | PRKCZ(5590); TNFRSF1B(7133); NUDC(10726); SESN2(83667); RBBP4 |
| hsa-miR-302d | 458; 459; | (5928); MACF1(23499); PLK3(1263); SH3GLB1(51100); CDC14A(8556); SYCP1 |
| | 460; 461; 462; | (6847); BCL9(607); BNIPL(149428); DAP3(7818); PEA15(8682); UHMK1 |
| | 463; 464; | (127933); FASLG(356); IPO9(55705); CIAS1(114548); TNFRSF14(8764); |
| | 465; 23; 466; | TNFRSF9(3604); DFFA(1676); E2F2(1870); CLSPN(63967); GFI1(2672); |
| | 467; 468; | SASS6(163786); MCL1(4170); IL10(3586); YOD1(55432); TP53BP2 |
| | 469; 470; | (7159); FH(2271); RHOB(388); BIRC6(57448); TCF7L1(83439); BRRN1(23397); |
| | 471; 472; 473; | BCL2L11(10018); RIF1(55183); CYBRD1(79901); ZAK(51776); CFLAR |
| | 474; 475; | (8837); CASP8(841); CD28(940); YWHAQ(10971); TGFA(7039); RNF149 |
| | 862; 863; | (284996); ANAPC1(64682); MCM6(4175); IFIH1(64135); NCKAP1(10787); |
| | 864; 865; 866; | STK17B(9262); CUL3(8452); HDAC4(9759); FANCD2(2177); PCAF(8850); |
| | 867; 868; | MLH1(4292); MCM2(4171); SENP2(59343); TP73L(8626); NME6(10201); |
| | 869; 870; | STAG1(10274); PIK3CB(5291); MGC40579(256356); ATR(545); TNFSF10 |
| | 871; 872; 873; | (8743); HD(3064); CD38(952); IL8(3576); SEPT11(55752); CCNG2 |
| | 874; 875; | (901); FGF2(2247); TLR2(7097); APBB2(323); LEF1(51176); CASP6(839); |
| | 876; 877; | LOC152485(152485); KIAA1909(153478); NIPBL(25836); MRPS30(10884); |
| | 878; 879; 880; | ERBB2IP(55914); RAD17(5884); F2R(2149); APC(324); RAD50(10111); |
| | 881; 2225; | TCF7(6932); ERGIC1(57222); BNIP1(662); PRKAA1(5562); BIRC1(4671); |
| | | MCC(4163); ATG12(9140); IRF1(3659); PPP2CA(5515); CDC23(8697); CD14 |
| | | (929); E2F3(1871); GMNN(51053); PARC(23113); VEGF(7422); PTP4A1 |
| | | (7803); FOXO3A(2309); SASH1(23328); IER3(8870); NUDT3(11165); CUL7 |
| | | (9820); MCM3(4172); DST(667); ATG5(9474); BCLAF1(9774); PDCD2 |
| | | (5134); AHR(196); EGFR(1956); CROT(54677); ZKSCAN1(7586); BCAP29 |
| | | (55973); CUL1(8454); TRIAD3(54476); PMS2(5395); CYCS(54205); CARD4 |
| | | (10392); ELMO1(9844); INHBA(3624); TMED4(222068); HIP1(3092); |
| | | CDK6(1021); TNFRSF10C(8794); BNIP3L(665); ESCO2(157570); FZD3 |
| | | (7976); UNC5D(137970); UBE2V2(7336); E2F5(1875); FZD6(8323); TNFRSF10B |
| | | (8795); TNFRSF10D(8793); TNFRSF10A(8797); DKK4(27121); TP53INP1 |
| | | (94241); TRPS1(7227); EXT1(2131); ANKRD15(23189); C9orf47 |
| | | (286223); CDKN2B(1030); TAF1L(138474); BAG1(573); SEMA4D(10507); |
| | | PTCH(5727); CTNNAL1(8727); TNFSF15(9966); TRAF1(7185); PPP6C |
| | | (5537); CIZ1(25792); TSC1(7248); ZMYND11(10771); C10orf9(219771); PCGF5 |
| | | (84333); KIF11(3832); BTRC(8945); PDCD11(22984); CSPG6(9126); |
| | | FRMD4A(55691); POLR3A(11128); SMNDC1(10285); C10orf46(143384); |
| | | MKI67(4288); BNIP3(664); STIM1(6786); WEE1(7465); HIPK3(10114); LGALS12 |
| | | (85329); CCND1(595); BIRC2(329); ATM(472); LOH11CR2A(4013); |
| | | HRASLS3(11145); MAP3K11(4296); RELA(5970); FZD4(8322); ICEBERG |
| | | (59082); PPP2R1B(5519); H2AFX(3014); CCND2(894); CNAP1(9918); |
| | | IAPP(3375); FGD4(121512); GDF11(10220); RAN(5901); HDAC7A(51564); |
| | | FAIM2(23017); PAWR(5074); BTG1(694); DIP13B(55198); ANAPC7(51434); |
| | | ANAPC5(51433); RFP2(10206); LATS2(26524); LIG4(3981); PNN(5411); |
| | | PSEN1(5663); GZMH(2999); NEK9(91754); SPTLC2(9517); CHES1(1112); |
| | | MOAP1(64112); AKT1(207); FGF7(2252); IGF1R(3480); NDN(4692); |
| | | BMF(90427); DAPK2(23604); CSNK1G1(53944); DNASE1(1773); PLK1 |
| | | (5347); SPN(6693); CARD15(64127); RBL2(5934); MARVELD3(91862); CDT1 |
| | | (81620); ERN2(10595); DNAJA2(10294); FTS(64400); SMPD3(55512); |
| | | ALOX15B(247); MAPK7(5598); TRAF4(9618); NF1(4763); FZD2(2535); |
| | | MAPT(4137); CROP(51747); PRR11(55771); PPM1D(8493); TLK2(11011); |
| | | KPNA2(3838); BIRC5(332); MNT(4335); GAS7(8522); SPAG5(10615); BRCA1 |
| | | (672); CSNK1D(1453); CABLES1(91768); MAPRE2(10982); MALT1 |
| | | (10892); TXNL1(9352); TXNL4A(10907); CHAF1A(10036); TNFAIP8L1(126282); |
| | | UHRF1(29128); PIK3R2(5296); CCNE1(898); PDCD5(9141); ATF5 |
| | | (22809); SPIB(6689); TNFSF14(8740); CDC37(11140); ZNF443(10224); |
| | | DNASE2(1777); SIRT2(22933); OPA3(80207); CARD8(22900); KLK10(5655); |
| | | POFUT1(23509); CEP250(11190); STK4(6789); E2F1(1869); RBL1(5933); |
| | | ELMO2(63916); DIDO1(11083); USP16(10600); SON(6651); APP(351); |
| | | SNF1LK(150094); ITGB2(3689); NF2(4771); SEPT3(55964); PPARA(5465); |
| | | BID(637); MAPK1(5594); CARD10(29775); DMC1(11144); TOB2(107 |

Table 4-13

| | | |
|---|---|---|
| | | 66); SMC1L2(27127); MAGEH1(28986); TAF1(6872); AGTR2(186); BIRC4 |
| | | (331); STAG2(10735); CD40LG(959); ARHGEF6(9459); BCAP31(10134); |
| | | MTCP1(4515); |
| hsa-miR-95 | 24; 476; 477; | B3GALT6(126792); C1orf188(148646); UBE4B(10277); DNAJC16(23341); |
| | 478; 479; | UBXD3(127733); ARID1A(8289); SESN2(83667); YBX1(4904); TMEM125 |
| | 480; 481; 482; | (128218); C1orf84(149469); CCDC76(54482); SLC6A17(388662); SYCP1 |
| | | (6847); FDPS(2224); CACNA1E(777); C1orf57(84284); KMO(8564); MYOM3 |
| | | (127294); SLC35D1(23169); EVI5(7813); ARHGAP29(9411); C1orf183 |
| | | (55924); TRIM33(51592); RP5-998N21.6(440686); RP11-196G18.6(440689); |
| | | TDRKH(11022); TPM3(7170); HDGF(3068); CD3Z(919); NME7(29922); |
| | | GNG4(2786); SP5(389058); C2orf17(79137); FLJ32447(151278); RHBDD1 |
| | | (84236); DGKD(8527); WDR35(57539); TGOLN2(10618); NR4A2(4929); |
| | | IL8RA(3577); MLH1(4292); RNF123(63891); RBM15B(29890); BOC |
| | | (91653); FBXO40(51725); IL1RAP(3556); SUMF1(285362); RAD18(56852); |
| | | NUP210(23225); DALRD3(55152); USP4(7375); RFT1(91869); FLJ44076 |
| | | (401080); MGLL(11343); STAG1(10274); WHSC1(7468); HD(3064); TBC1D14 |
| | | (57533); FLJ35725(152992); NPAL1(152519); CCNG2(901); QDPR |
| | | (5860); LGI2(55203); CHIC2(26511); ADAMTS3(9508); UNC5C(8633); ADH5 |
| | | (128); PLA2G12A(81579); FLJ23191(79625); POLS(11044); RAI14(26064); |
| | | ZNF131(7690); MATR3(9782); NDST1(3340); CANX(821); LIFR(3977); |
| | | GCNT2(2651); NOL7(51406); RNF8(9025); BAG2(9532); DCBLD1(285761); |
| | | C6orf75(60487); AKAP12(9590); HMGCLL1(54511); BVES(11149); |
| | | PDSS2(57107); BCLAF1(9774); EIF3S9(8662); FOXK1(221937); HDAC9 |
| | | (9734); CPSF4(10898); DUS4L(11062); DRCTNNB1A(84668); CYCS(54205); |
| | | AAA1(404744); NUDCD3(23386); CALN1(83698); NCF1(4687); CDK6 |
| | | (1021); FLJ10925(55262); CLEC5A(23601); KBTBD11(9920); ZDHHC2(51201); |
| | | SPFH2(11160); PHF20L1(51105); MAPK15(225689); DUSP4(1846); |
| | | PURG(29942); GDF6(392255); RAD21(5885); DDEF1(50807); C9orf89 |
| | | (84270); RABGAP1(23637); C9orf28(89853); FLJ36779(158055); EHMT1 |
| | | (79813); IPPK(64768); PTPN3(5774); TSC1(7248); GATA3(2625); CUGBP2 |
| | | (10659); C10orf47(254427); ZNF365(22891); SUFU(51684); PDCD4(27250); |
| | | PTPRE(5791); IL2RA(3559); PCBD1(5092); KIAA1754(85450); C11orf17 |
| | | (56672); NAV2(89797); GAS2(2620); ZCSL3(120526); FJX1(24147); C11orf9 |
| | | (745); ZFPL1(7542); LRFN4(78999); LRP5(4041); CCND1(595); ARHGEF17 |
| | | (9828); FUT4(2526); CUL5(8065); DUSP8(1850); CTSD(1509); IMMP1L |
| | | (196294); STARD10(10809); CD3D(915); CDON(50937); P53AIP1 |
| | | (63970); EMP1(2012); CUTL2(23316); PTPN11(5781); TCF1(6927); BCL7A |
| | | (605); BRI3BP(140707); EP400(57634); SCNN1A(6337); MGC24039(160518); |
| | | MGAT4C(25834); SLC41A2(84102); FBXO21(23014); PITPNM2 |
| | | (57605); CDK8(1024); COL4A2(1284); C13orf18(80183); RCBTB1(55213); |
| | | KIAA1008(22894); LRP10(26020); GPR65(8477); CALM1(801); CYP46A1 |
| | | (10858); WDR20(91833); TRAF3(7187); AMN(81693); TM9SF1(10548); EXOC5 |
| | | (10640); SEL1L(6400); PTPN21(11099); DICER1(23405); C14orf139 |
| | | (79686); CRI1(23741); CGNL1(84952); SNX1(6642); IL16(3603); ALPK3 |
| | | (57538); IGF1R(3480); LOC400464(400464); ARPP-19(10776); PRTG(283659); |
| | | PDCD7(10081); GRAMD2(196996); LOC57146(57146); DUS2L(54920); |
| | | CMIP(80790); PARN(5073); LOC283849(283849); MGC16385(92806); |
| | | RPA1(6117); PAFAH1B1(5048); LIG3(3980); NSF(4905); NXPH3(11248); |
| | | KPNA2(3838); FLJ35767(400629); TRPV3(162514); NALP1(22861); PRR6 |
| | | (201161); POLDIP2(26073); HCP1(113285); C18orf43(10650); MALT1 |
| | | (10892); DYM(54808); RAX(30062); STK11(6794); FLJ11286(55337); PDE4A |
| | | (5141); DNM2(1785); FOSB(2354); SPHK2(56848); LIN7B(64130); SPIB |
| | | (6689); LMNB2(84823); UBXD1(80700); FLJ25758(497049); PLVAP(83483); |
| | | ZNF708(7562); FLJ38451(126375); BCAT2(587); SIGLEC11(114132); |
| | | ZNF417(147687); SMOX(54498); MCM8(84515); PLCB1(23236); SLC24A3 |
| | | (57419); HM13(81502); MYLK2(85366); SLC12A5(57468); NTSR1(4923); |
| | | TCEA2(6919); YTHDF1(54915); BTBD4(140685); TTC3(7267); BACE2 |
| | | (25825); BTG3(10950); APP(351); MPST(4357); LRP5L(91355); MYH9(4627); |
| | | MKL1(57591); NHP2L1(4809); PHF21B(112885); MAGED1(9500); |
| | | EDA(1896); MAGED4(81557); |
| hsa-miR-98 | 25; 483; 484; | TNFRSF1B(7133); RBBP4(5928); MACF1(23499); PLK3(1263); BCL9(607); |
| | 485; 486; | DAP3(7818); PEA15(8682); FASLG(356); ADORA1(134); MDM4(4194); |
| | 487; 488; 489; | CIAS1(114548); E2F2(1870); PTAFR(5724); MUTYH(4595); FAF1(11124); |
| | 490; 491; | NRAS(4893); DEDD(9191); RHOB(388); BIRC6(57448); HK2(3099); BCL2L11 |
| | 492; | (10018); PROC(5624); FZD7(8324); CD28(940); E2F6(1876); CGRE |

Table 4-14

| | | |
|---|---|---|
| | | F1(10669); PPM1G(5496); TIA1(7072); SEPT10(151011); ANAPC1(64682); |
| | | PCAF(8850); ACPP(55); RNF7(9616); TP73L(8626); AXUD1(64651); NME6 |
| | | (10201); ATR(545); TNFSF10(8743); CCNG2(901); VEGFC(7424); CASP3 |
| | | (836); SKP2(6502); F2R(2149); CSNK1G3(1456); UNC5A(90249); PRLR |
| | | (5618); BIRC1(4671); ATG12(9140); CDC25C(995); HDAC3(8841); CDKN1A |
| | | (1026); VEGF(7422); LIN28B(389421); TNFAIP3(7128); MDC1(9656); |
| | | DST(667); MAP3K5(4217); PLAGL1(5325); AHR(196); FZD1(8321); |
| | | STAG3(10734); HBP1(26959); BCAP29(55973); CUL1(8454); MAD1L1(8379); |
| | | TRIAD3(54476); CARD4(10392); FASTK(10922); POLR3D(661); FZD3 |
| | | (7976); E2F5(1875); BIN3(55909); TNFRSF10A(8797); DUSP4(1846); |
| | | MOS(4342); CCNE2(9134); TP53INP1(94241); NUDT2(318); UNC13B(10497); |
| | | DAPK1(1612); BAG1(573); SEMA4D(10507); PTCH(5727); TNFSF15 |
| | | (9966); TNFSF8(944); CIZ1(25792); TSC1(7248); C10orf9(219771); FAS |
| | | (355); TCF7L2(6934); BAG3(9531); PARD3(56288); FZD8(8325); FBXW4 |
| | | (6468); DCLRE1A(9937); DPF2(5977); RPS6KB2(6199); FADD(8772); BIRC3 |
| | | (330); CHEK1(1111); RHOG(391); EIF4G2(1982); DKK3(27122); CDCA5 |
| | | (113130); PPP1CA(5499); PAK1(5058); GAB2(9846); FZD4(8322); SESN3 |
| | | (143686); CASP4(837); CASP5(838); CASP1(834); PPP2R1B(5519); BCL9L |
| | | (283149); H2AFX(3014); CNAP1(9918); TEGT(7009); ESPL1(9700); HMGA2 |
| | | (8091); MDM2(4193); CCT2(10576); APAF1(317); FZD10(11211); BCAT1 |
| | | (586); FAIM2(23017); LOC401720(401720); CDK4(1019); DIP13B(55198); |
| | | FGF9(2254); CDK8(1024); RB1(5925); RFP2(10206); NEK3(4752); |
| | | PNN(5411); C14orf28(122525); RAB15(376267); MAPK6(5597); IGF1R(3480); |
| | | AVEN(57099); SLC12A6(9990); BCL2L10(10017); DAPK2(23604); PUNC |
| | | (9543); TSC2(7249); CCNF(899); DNASE1(1773); PLK1(5347); CYLD |
| | | (1540); E2F4(1874); AXIN1(8312); GSPT1(2935); P8(26471); SMPD3(55512); |
| | | TERF2(7014); HIC1(3090); ALOX12(239); ALOX15B(247); MAPK7 |
| | | (5598); NLK(51701); NF1(4763); TADA2L(6871); IGF2BP1(10642); KPNA2 |
| | | (3838); MAP2K6(5608); SLC9A3R1(9368); CDK3(1018); SPHK1(8877); |
| | | MNT(4335); P2RX1(5023); TP53(7157); AURKB(9212); GAS7(8522); LIMD2 |
| | | (80774); CSNK1D(1453); MAPRE2(10982); MAPK4(5596); DCC(1630); |
| | | PHLPP(23239); BCL2(596); CDC34(997); FZR1(51343); TNFSF9(8744); |
| | | MAP3K10(4294); ARHGEF1(9138); APOE(348); SPHK2(56848); BCL2L12 |
| | | (83596); NALP2(55655); AES(166); MATK(4145); ERCC2(2068); OPA3 |
| | | (80207); BBC3(27113); LIG1(3978); KLK10(5655); PLCB1(23236); CTNNBL1 |
| | | (56259); PARD6B(84612); BCL2L1(598); AURKA(6790); DIDO1(11083); |
| | | GM632(57473); TRPM2(7226); ITGB2(3689); HIC2(23119); SMARCB1 |
| | | (6598); MAPK1(5594); MN1(4330); PDGFB(5155); TOB2(10766); HDAC6 |
| | | (10013); IKBKG(8517); SMC1L1(8243); CUL4B(8450); PDCD8(9131); HCFC1 |
| | | (3054); MTCP1(4515); |
| hsa-miR-130a | 26; 493; 494; | ATAD3C(219293); TNFRSF1B(7133); C1orf144(26099); CDC42(998); NUDC |
| hsa-miR-130b | 495; 496; | (10726); SESN2(83667); EIF2C3(192669); MACF1(23499); CDKN2C |
| hsa-miR-301 | 497; 498; 499; | (1031); MIER1(57708); GADD45A(1647); SH3GLB1(51100); CDC14A(8556); |
| | 500; 501; | CSF1(1435); RAP1A(5906); PEA15(8682); RALGPS2(55103); MDM4(4194); |
| | 502; 503; | G0S2(50486); TGFB2(7042); FBXO28(23219); TNFRSF9(3604); DFFA |
| | 504; 505; 27; | (1676); E2F2(1870); TESK2(10420); DEDD(9191); ASPM(259266); ANGEL2 |
| | 506; 507; | (90806); MAPRE3(22924); BIRC6(57448); MXD1(4084); HTRA2(27429); |
| | 508; 509; 510 | TCF7L1(83439); BRRN1(23397); EPB41L5(57669); ZAK(51776); CASP8 |
| | 28; 511; | (841); CREB1(1385); COL4A3(1285); PSMD1(5707); RTN4(57142); TGFA |
| | 512; 513; 514; | (7039); EDAR(10913); ANAPC1(64682); FRZB(2487); NCKAP1(10787); |
| | 515; 516; | CUL3(8452); APPL(26060); NEK11(79858); SMC4L1(10051); FXR1(8087); |
| | 517; 518; | TP73L(8626); AXUD1(64651); NME6(10201); RASSF1(11186); LRIG1 |
| | 519; 520; 521; | (26018); UBE1C(9039); GSK3B(2932); TFDP2(7029); ATR(545); EIF5A2 |
| | 882; 883; | (56648); TNFSF10(8743); HCAP-G(64151); EREG(2069); SEPT11(55752); |
| | 884; 885; | CCNG2(901); HERC3(8916); NFKB1(4790); TRIM2(23321); LRPAP1(4043); |
| | 886; 2226; | GABRA4(2557); CCNI(10983); CASP6(839); ERBB2IP(55914); CCNB1 |
| | | (891); LOC91137(91137); APC(324); TCF7(6932); UNC5A(90249); PRKAA1 |
| | | (5562); MCC(4163); ATG12(9140); SEMA6A(57556); IRF1(3659); CD14 |
| | | (929); FBXW11(23291); TNF(7124); CDKN1A(1026); CDC5L(988); PTP4A1 |
| | | (7803); HECA(51696); SASH1(23328); MCM3(4172); DST(667); SESN1 |
| | | (27244); FRK(2444); MAP3K5(4217); STK17A(9263); ADCY1(107); EGFR |
| | | (1956); HBP1(26959); BCAP29(55973); CASP2(835); TRIAD3(54476); CYCS |
| | | (54205); ELMO1(9844); INHBA(3624); STEAP4(79689); FLJ31818(154743); |
| | | FASTK(10922); MTMR9(66036); ESCO2(157570); UNC5D(137970); |
| | | TACC1(6867); SULF1(23213); FZD6(8323); MYC(4609); TNFRSF10B(8 |

Table 4-15

| | | |
|---|---|---|
| | | 795); DKK4(27121); TP53INP1(94241); STK3(6788); RECK(8434); INVS |
| | | (27130); ABL1(25); PTCH(5727); TNFSF15(9966); TNFSF8(944); TSC1(7248); |
| | | C10orf9(219771); PTEN(5728); KIF11(3832); BTRC(8945); DOCK1(1793); |
| | | FLJ45187(387640); DLG5(9231); FBXW4(6468); FGF8(2253); IFITM1 |
| | | (8519); WEE1(7465); HIPK3(10114); API5(8539); FBXL11(22992); CUL5 |
| | | (8065); ATM(472); PTH(5741); PGM2L1(283209); DLG2(1740); FZD4(8322); |
| | | SESN3(143686); ICEBERG(59082); PPP2R1B(5519); IL18(3606); DDX6 |
| | | (1656); BCL9L(283149); CCND2(894); CNAP1(9918); BCL2L14(79370); |
| | | GOLT1B(51026); SOCS2(8835); APAF1(317); CHST11(50515); RAN |
| | | (5901); LRP6(4040); BCAT1(586); FAIM2(23017); CDK4(1019); BTG1(694); |
| | | FLJ20674(54621); MPHOSPH9(10198); HSMPP8(54737); FGF9(2254); |
| | | TNFRSF19(55504); RB1(5925); SUGT1(10910); FLJ30046(122060); ING1 |
| | | (3621); CGRRF1(10668); PSEN1(5663); VASH1(22846); GPR65(8477); DAD1 |
| | | (1603); STRN3(29966); EGLN3(112399); CDKL1(8814); KIAA0831(22863); |
| | | NEK9(91754); RPS6KA5(9252); DICER1(23405); BAG5(9529); JAG2 |
| | | (3714); CSK(1445); SLC12A6(9990); CSNK1G1(53944); CCNF(899); CYLD |
| | | (1540); GSPT1(2935); USP31(57478); FTS(64400); DYNC1LI2(1783); |
| | | TERF2(7014); PAFAH1B1(5048); BRCA1(672);PTRH2(51651); ERN1(2081); |
| | | CSNK1D(1453); CABLES1(91768); GADD45B(4616); DNM2(1785); |
| | | LDLR(3949); UBA52(7311); SPHK2(56848); DEDD2(162989); PPP1R13L |
| | | (10848); KLK10(5655); PLCB1(23236); CEP250(11190); STK4(6789); CSNK2A1 |
| | | (1457); PCNA(5111); RBL1(5933); BRWD1(54014); SNF1LK(150094); |
| | | KREMEN1(83999); GAS2L1(10634); NF2(4771); PGEA1(25776); MAPK1 |
| | | (5594); PPM1F(9647); CHEK2(11200); SMC1L2(27127); PCTK1(5127); |
| | | NGFRAP1(27018); BIRC4(331); DKC1(1736); PIGA(5277); ARHGEF6 |
| | | (9459); MECP2(4204); |
| hsa-miR-132 | 29; 522; 523; | H6PD(9563); PIK3CD(5293); EPHB32(2048); RCC1(1104); LCK(3932); HDAC1 |
| hsa-miR-212 | 524; 525; | (3065); EIF2C1(26523); MACF1(23499); SLC35A3(23443); CDC14A |
| | 526; 527; 528; | (8556); PEA15(8682); UHMK1(127933); SOAT1(6646); TMEM58(149345); |
| | 529; 30; | ELF3(1999); PPP1R12B(4660); KCNK2(3776); TGFB2(7042); FBXO28(23219); |
| | 530; 531; 532; | TNFRSF9(3604); DFFA(1676); ZBTB17(7709); FBXO42(54455); PPT1 |
| | 533; 534; | (5538); DAB1(1600); NOTCH2(4853); DEDD(9191); NMNAT2(23057); |
| | 535; 536; | ESRRG(2104); SOX11(6664); MGC11266(79172); BIRC6(57448); MSH2 |
| | | (4436); HSPC159(29094); KCMF1(56888); TCF7L1(83439); KCNIP3(30818); |
| | | BCL2L11(10018); TAIP-2(80034); ZAK(51776); NAB1(4664); COL4A3 |
| | | (1285); PSMD1(5707); ASB1(51665); SMC6L1(79677); PPM1G(5496); CARD12 |
| | | (58484); RTN4(57142); TIA1(7072); LRRTM4(80059); TMEM127 |
| | | (55654); MGC52110(493753); BUB1(699); ANAPC1(64682); WDR33(55339); |
| | | ORC4L(5000); KLF7(8609); IGFBP5(3488); USP37(57695); PAX3(5077); |
| | | COL4A4(1286); TRIP12(9320); VHL(7428); SLC6A1(6529); NR2C2(7182); |
| | | PDCD6IP(10015); NKTR(4820); APPL(26060); PCNP(57092); SLC15A2 |
| | | (6565); MCM2(4171); NEK11(79858); ARMC8(25852); FAIM(55179); FXR1 |
| | | (8087); SENP2(59343); FAM79B(285386); SRGAP3(9901); SEC22L3 |
| | | (9117); LTF(4057); SLC25A20(788); C3orf62(375341); TUSC2(11334); RASSF1 |
| | | (11186); SFMBT1(51460); PRICKLE2(166336); DHFRL1(200895); RABL3 |
| | | (285282); SEPT11(55752); PHF17(79960); HHIP(64399); SLBP(7884); |
| | | APBB2(323); GRSF1(2926); CCNI(10983); PIGY(84992); NIPBL(25836); |
| | | CARD6(84674); R7BP(401190); ERBB2IP(55914); PIK3R1(5295); SCAMP1 |
| | | (9522); RASA1(5921); APC(324); RAD50(10111); FLJ40453(401217); |
| | | DHFR(1719); SEMA6A(57556); RAPGEF6(51735); IRF1(3659); HBEGF |
| | | (1839); YIPF5(81555); CSNK1A1(1452); PRPF4B(8899); IBRDC2(255488); |
| | | SOX4(6659); GMNN(51053); CDKN1A(1026); TTK(7272); FOXO3A(2309); |
| | | PLEKHG1(57480); ARID1B(57492); ELOVL2(54898); NEDD9(4739); |
| | | MDC1(9656); GUCA1B(2979); DST(667); SESN1(27244); FRK(2444); HBS1L |
| | | (10767); MAP3K5(4217); SOD2(6648); LPAL2(80350); PDCD2(5134); |
| | | TMEM106B(54664); CREB5(9586); EGFR(1956); FZD1(8321); HBP1(26959); |
| | | CBLL1(79872); CALU(813); SOSTDC1(25928); JAZF1(221895); ELMO1 |
| | | (9844); NUDCD3(23386); PURB(5814); YWHAG(7532); MCM7(4176); |
| | | TACC1(6867); SGK3(23678); E2F5(1875); RIPK2(8767); SLC26A7(115111); |
| | | FZD6(8323); TRIB1(10221); PHF20L1(51105); TNFRSF10B(8795); ADRA1A |
| | | (148); TMEM64(169200); TSPYL5(85453); AZIN1(51582); RAD21 |
| | | (5885); C9orf47(286223); SLC44A1(23446); BNC2(54796); TAF1L(138474); |
| | | GOLPH2(51280); TSC1(7248); BRD3(8019); SIRT1(23411); FAS(355); KIF11 |
| | | (3832); BTRC(8945); SUFU(51684); PDCD11(22984); DOCK1(1793); |
| | | ZWINT(11130); C10orf46(143384); GTF2H1(2965); HIPK3(10114); GPIA |

Table 4-16

| | | |
|---|---|---|
| | | P1(4076); EHF(26298); API5(8539); BIRC3(330); CUL5(8065); DKK3(27122); |
| | | WT1(7490); CD59(966); SUV420H1(51111); FZD4(8322); FAM76B(143684); |
| | | PPP2R1B(5519); KCNA6(3742); NTF3(4908); CNAP1(9918); PDE3A |
| | | (5139); ARID2(196528); KIF5A(3798); MARCH9(92979); LEMD3(23592); |
| | | MSRB3(253827); DYRK2(8445); MDM2(4193); SOCS2(8835); SLC25A3 |
| | | (5250); APAF1(317); BCAT1(586); KRAS(3845); OSBPL8(114882); PPP1CC |
| | | (5501); SPPL3(121665); ZNF605(90462); RB1(5925); C13orf6(84945); |
| | | TFDP1(7027); SLC7A1(6541); C13orf10(64062); BCL2L2(599); PNN(5411); |
| | | TXNDC(81542); FRMD6(122786); CGRRF1(10668); DAAM1(23002); PPP2R5C |
| | | (5527); STRN3(29966); GMFB(2764); TIMM9(26520); ENTPD5 |
| | | (957); LTBP2(4053); CHES1(1112); BUB1B(701); CHP(11261); FGF7(2252); |
| | | MAPK6(5597); Gcom1(145781); FBXO22(26263); TMEM87A(25963); FRMD5 |
| | | (84978); BNIP2(663); CSNK1G1(53944); AP3S2(10239); CYLD(1540); |
| | | RBL2(5934); NFAT5(10725); DCUN1D3(123879); MAPK3(5595); DNAJA2 |
| | | (10294); SMPD3(55512); MIS12(79003); GOSR1(9527); LIG3(3980); |
| | | MGC19764(162394); RND2(8153); MAPT(4137); NGFR(4804); TMEM49 |
| | | (81671); PRKCA(5578); CRK(1398); MNT(4335); GPS2(2874); GAS7(8522); |
| | | SSH2(85464); BRCA1(672); HDAC5(10014); PHB(5245); CABLES1(91768); |
| | | IER3IP1(51124); MBP(4155); CYP4F3(4051); KCTD15(79047); PSMD8 |
| | | (5714); FOSB(2354); ZNF585A(199704); FLJ36888(126526); LAIR1(3903); |
| | | TRIB3(57761); SNRPB2(6629); CBFA2T2(9139); PPP1R16B(26051); |
| | | STK4(6789); RBL1(5933); UBE2V1(7335); AURKA(6790); CTCFL(140690); |
| | | DIDO1(11083); SON(6651); AGPAT3(56894); BRWD1(54014); PRDM15 |
| | | (63977); SEPT5(5413); EP300(2033); ADPN(80339); GTSE1(51512); MAPK1 |
| | | (5594); GNL3L(54552); TAF1(6872); RP11-321G1.1(54885); IKBKG |
| | | (8517); SMC1L1(8243); PCDH19(57526); IDS(3423); |
| hsa-miR-133a | 31; 537; 538; | CDC42(998); SESN2(83667); KHDRBS1(10657); RBBP4(5928); PLK3(1263); |
| hsa-miR-133b | 539; 540; | CDC14A(8556); BCL9(607); BNIPL(149428); ADORA1(134); G0S2(50486); |
| | 541; 542; 543; | CDC2L2(985); NADK(65220); TNFRSF14(8764); FRAP1(2475); CASP9 |
| | 544; 545; | (842); RCC2(55920); E2F2(1870); WASF2(10163); CLSPN(63967); MUTYH |
| | 546; 547; | (4595); TESK2(10420); GFI1(2672); FOSL2(2355); BIRC6(57448); |
| | 548; 549; 32; | GKN1(56287); DOK1(1796); HK2(3099); KCNIP3(30818); BRRN1(23397); |
| | 550; 551; | ZAK(51776); CFLAR(8837); CASP10(843); INHA(3623); COL4A3(1285); |
| | 2227; | PPM1G(5496); TIA1(7072); TGFA(7039); SEPT10(151011); IL1B(3553); |
| | | TLK1(9874); STK17B(9262); PER2(8864); HDAC4(9759); KIF1A(547); PDCD6IP |
| | | (10015); APPL(26060); TRAIP(10293); RASSF1(11186); DNASE1L3 |
| | | (1776); TNFSF10(8743); HD(3064); SPP1(6696); NFKB1(4790); FGF2 |
| | | (2247); GAK(2580); DKK2(27123); MAD2L1(4085); VEGFC(7424); FGF10 |
| | | (2255); MCC(4163); NME5(8382); FGF1(2246); DUSP1(1843); TCF19(6941); |
| | | LTA(4049); CDKN1A(1026); PARC(23113); CDC5L(988); FOXO3A(2309); |
| | | TNFAIP3(7128); SASH1(23328); C6orf149(57128); MDC1(9656); IHPK3 |
| | | (117283); CCND3(896); CUL7(9820); TNFRSF21(27242); MCM3(4172); |
| | | CCNC(892); SESN1(27244); PLAGL1(5325); HDAC9(9734); ADCY1(107); |
| | | FZD1(8321); CUL1(8454); MAD1L1(8379); TRIAD3(54476); PMS2(5395); |
| | | CARD4(10392); INHBA(3624); BRAF(673); BNIP3L(665); UNC5D(137970); |
| | | RIPK2(8767); PINX1(54984); BIN3(55909); TNFRSF10B(8795); TNFRSF10A |
| | | (8797); ADRA1A(148); TRIM35(23087); CLU(1191); DUSP4(1846); |
| | | SFRP1(6422); MOS(4342); STK3(6788); TNFRSF11B(4982); MTSS1 |
| | | (9788); UNC13B(10497); DAPK1(1612); ABL1(25); TRAF2(7186); TAF1L |
| | | (138474); GAS1(2619); SEMA4D(10507); TNFSF15(9966); TNFSF8(944); |
| | | TSC1(7248); CARD9(64170); ANAPC2(29882); SGPL1(8879); KIF11(3832); |
| | | SUFU(51684); PDCD11(22984); DLG5(9231); DCLRE1A(9937); C10orf46 |
| | | (143384); MKI67(4288); EXT2(2132); MDK(4192); INCENP(3619); LGALS12 |
| | | (85329); RPS6KB2(6199); LRP5(4041); BIRC3(330); ATM(472); CHEK1 |
| | | (1111); HRAS(3265); APBB1(322); DKK3(27122); MAP3K11(4296); RELA |
| | | (5970); FGF3(2248); LTBR(4055); TNFRSF7(939); CNAP1(9918); PTPN6 |
| | | (5777); BCL2L14(79370); TEGT(7009); TNFRSF1A(7132); LRP6(4040); |
| | | BCAT1(586); KRAS(3845); HDAC7A(51564); MCRS1(10445); FAIM2(23017); |
| | | C12orf22(81566); PCTK2(5128); DIP13B(55198); HSMPP8(54737); |
| | | TFDP1(7027); BCL2L2(599); C14orf115(55237); LTBP2(4053); BAG5(9529); |
| | | PPP1R13B(23368); AKT1(207); IGF1R(3480); AVEN(57099); BMF(90427); |
| | | DAPK2(23604); RAB40C(57799); TRAF7(84231); KREMEN2(79412); |
| | | NOL3(8996); GSPT1(2935); PYCARD(29108); CSNK2A2(1459); SLC12A4 |
| | | (6560); SMPD3(55512); TUSC5(286753); HIC1(3090); ALOX15B(247); |
| | | MAPK7(5598); LASP1(3927); NGFR(4804); CROP(51747); NME1(4830) |

Table 4-17

| | | |
|---|---|---|
| | | ; PPM1D(8493); PRKCA(5578); SLC9A3R1(9368); CARD14(79062); MNT |
| | | (4335); P2RX1(5023); NALP1(22861); TP53(7157); GAS7(8522); AP1GBP1 |
| | | (11276); HDAC5(10014); MPO(4353); LIMD2(80774); TMC6(11322); SOCS3 |
| | | (9021); CETN1(1068); MAPRE2(10982); PHLPP(23239); CDC34(997); |
| | | CSNK1G2(1455); TNFSF9(8744); DNM2(1785); SYDE1(85360); CCNE1 |
| | | (898); PSMD8(5714); AXL(558); ARHGEF1(9138); BCL3(602); APOE(348); |
| | | SPHK2(56848); MATK(4145); DAPK3(1613); CDC37(11140); TGFB1(7040); |
| | | ERCC2(2068); LIG1(3978); CEP250(11190); NNAT(4826); MYBL2(4605); |
| | | UBE2C(11065); PARD6B(84612); BIRC7(79444); BCL2L1(598); E2F1 |
| | | (1869); CTCFL(140690); DIDO1(11083); CHAF1B(8208); MX1(4599); APP |
| | | (351); ITGB2(3689); GAS2L1(10634); NF2(4771); GTSE1(51512); MAPK1 |
| | | (5594); PPM1F(9647); MN1(4330); CARD10(29775); TAF1(6872); DKC1 |
| | | (1736); PPP2R3B(28227); SEPT6(23157); HCFC1(3054); |
| hsa-miR-134 | 33; 552; 553; | CDC42(998); RCC1(1104); MACF1(23499); CDC20(991); PROK1(84432); |
| | 554; 555; | BCL9(607); UHMK1(127933); TRAF5(7188); TGFB2(7042); DFFA(1676); |
| | 556; 557; | FRAP1(2475); RCC2(55920); RIMS3(9783); NOTCH2(4853); ASPM(259266); |
| | | BIRC6(57448); HTRA2(27429); TCF7L1(83439); PROC(5624); RIF1 |
| | | (55183); TAIP-2(80034); ZAK(51776); TIA1(7072); TGFA(7039); HDAC4(9759); |
| | | PDCD6IP(10015); NEK4(6787); PIK3CB(5291); TFDP2(7029); WHSC1 |
| | | (7468); HCAP-G(64151); FGF2(2247); AFAP(60312); MFAP3L(9848); CARD6 |
| | | (84674); RAD17(5884); CSNK1G3(1456); RAD50(10111); DAP(1611); |
| | | PRLR(5618); BIRC1(4671); IRF1(3659); CD74(972); E2F3(1871); VEGF |
| | | (7422); BAG2(9532); PTP4A1(7803); TTK(7272); FOXO3A(2309); NEDD9 |
| | | (4739); MDC1(9656); DST(667); FRK(2444); BCLAF1(9774); MAP3K5(4217); |
| | | PLAGL1(5325); LATS1(9113); PDCD2(5134); HDAC9(9734); EGFR(1956); |
| | | STAG3(10734); PMS2(5395); SOSTDC1(25928); HIPK2(28996); FZD3 |
| | | (7976); UNC5D(137970); BAG4(9530); TACC1(6867); RIPK2(8767); PINX1 |
| | | (54984); ADRA1A(148); TRIM35(23087); DUSP4(1846); TP53INP1(94241); |
| | | ANKRD15(23189); ABL1(25); CDKN2A(1029); GRIN3A(116443); CTNNAL1 |
| | | (8727); TRAF1(7185); CIZ1(25792); TSC1(7248); BTRC(8945); PDCD11 |
| | | (22984); BAG3(9531); DOCK1(1793); C10orf63(219670); PARD3(56288); |
| | | DLG5(9231); MKI67(4288); STIM1(6786); WEE1(7465); MS4A2(2206); |
| | | FADD(8772); CUL5(8065); ATM(472); LOH11CR2A(4013); HRAS(3265); |
| | | BAD(572); CLCF1(23529); MTL5(9633); SESN6(143686); CNAP1(9918); |
| | | ESPL1(9700); SOCS2(8835); GAS2L3(283431); KNTC1(9735); RAN |
| | | (5901); BCAT1(586); KRAS(3845); FAIM2(23017); DDIT3(1649); DUSP6(1848); |
| | | PCTK2(5128); ANAPC7(51434); MPHOSPH9(10198); HSMPP8(54737); |
| | | FGF9(2254); TNFRSF19(55504); CDK8(1024); PSEN1(5663); VASH1 |
| | | (22846); TRAF3(7187); ACIN1(22985); DLG7(9787); LTBP2(4053); CHES1 |
| | | (1112); BUB1B(701); MAPK6(5597); KIF23(9493); AVEN(57099); PDCD7 |
| | | (10081); TSC2(7249); GNAO1(2775); CTCF(10664); GSPT1(2935); ERN2 |
| | | (10595); CIAPIN1(57019); SLC12A4(6560); MPHOSPH6(10200); PAFAH1B1 |
| | | (5048); TNFSF12(8742); MAPK7(5598); NF1(4763); CCL2(6347); |
| | | LIG3(3980); MAPT(4137); PRKCA(5578); NARF(26502); MNT(4335); NALP1 |
| | | (22861); TP53(7157); PTRH2(51651); SOCS3(9021); CABLES1(91768); |
| | | MAPRE2(10982); MALT1(10892); DYM(54808); PIN1(5300); APOE(348); |
| | | TNFSF14(8740); ZNF28(7576); CEP250(11190); CSE1L(1434); CSNK2A1 |
| | | (1457); AURKA(6790); ADORA2A(135); KREMEN1(83999); GAS2L1 |
| | | (10634); NF2(4771); GTSE1(51512); BID(637); CHEK2(11200); SSTR3(6753); |
| | | PDGFB(5155); TOB2(10766); SMC1L2(27127); IKBKG(8517); PPP2R3B |
| | | (28227); SPIN2(54466); SEPT6(23157); PDCD8(9131); ARHGEF6(9459); |
| | | HCFC1(3054); |
| hsa-miR-137 | 34; 558; 559; | DFFB(1677); CDC42(998); MACF1(23499); SH3GLB1(51100); RAP1A(5906); |
| | 560; | CD2(914); MDM4(4194); IL19(29949); PROX1(5629); TGFB2(7042); YARS |
| | | (8565); CLSPN(63967); RIMS3(9783); GFI1(2672); NOTCH2(4853); |
| | | TOR1AIP2(163590); ASPM(259266); YOD1(55432); ESRRG(2104); TP53BP2 |
| | | (7159); FH(2271); PLEKHH2(130271); PRKCE(5581); HK2(3099); RIF1 |
| | | (55183); TAIP-2(80034); CASP10(843); CASP8(841); CD28(940); COL4A3 |
| | | (1285); PSMD1(5707); CARD12(58484); EDAR(10913); ANAPC1(64682); |
| | | MCM6(4175); IFIH1(64135); TLK1(9874); FRZB(2487); CUL3(8452); LOH3CR2A |
| | | (29931); FANCD2(2177); NEK11(79858); SMC4L1(10051); FXR1 |
| | | (8087); SENP2(59343); RAF1(5894); NEK4(6787); ATR(545); TNFSF10 |
| | | (8743); MGC21675(92070); HD(3064); ANAPC4(29945); EREG(2069); SEPT11 |
| | | (55752); CCNG2(901); FGF5(2250); NFKB1(4790); ELMOD2(255520); |
| | | BTC(685); CASP6(839); MAD2L1(4085); SPATA4(132851); CASP3(83 |

Table 4-18

| | | |
|---|---|---|
| | | 6); PDCD6(10016); NIPBL(25836); ERBB21P(55914); CSNK1G3(1456); RAD50 |
| | | (10111); PRLR(5618); P18SRP(285672); BIRC1(4671); MCC(4163); |
| | | SEMA6A(57556); NRG2(9542); PFDN1(5201); FGF1(2246); LY86(9450); |
| | | MAPK13(5603); TTK(7272); ACY1L2(135293); AIM1(202); HECA(51696); |
| | | QKI(9444); NEDD9(4739); DAXX(1616); MCM3(4172); DST(667); SESN1 |
| | | (27244); SGK(6446); AHR(196); HDAC9(9734); EGFR(1956); FZD1(8321); |
| | | RINT1(60561); HBP1(26959); MKLN1(4289); CYCS(54205); CARD4(10392); |
| | | INHBA(3624); FGL2(10875); CDK6(1021); PTN(5764); VPS37A(137492); |
| | | UNC5D(137970); BAG4(9530); TACC1(6867); UBE2V2(7336); SULF1 |
| | | (23213); LZTS1(11178); DUSP4(1846); NCOA2(10499); TP53INP1(94241); |
| | | STK3(6788); CDC37L1(55664); RECK(8434); IFNW1(3467); CDKN2B |
| | | (1030); TAF1L(138474); CTNNAL1(8727); TNFSF8(944); ZMYND11(10771); |
| | | C10orf9(219771); SGPL1(8879); C10orf104(119504); KIF11(3832); CSPG6 |
| | | (9126); TCF7L2(6934); PARD3(56288); CUL2(8453); PPP3CB(5532); |
| | | COX15(1355); SMNDC1(10285); C10orf46(143384); TIAL1(7073); MKI67 |
| | | (4288); WEE1(7465); GAS2(2620); API5(8539); MDK(4192); INCENP(3619); |
| | | BIRC3(330); ATM(472); CD3G(917); LGR4(55366); HRASLS3(11145); |
| | | MAP3K11(4296); FZD4(8322); SESN3(143686); PPP2R1B(5519); SCN3B |
| | | (55800); CCND2(894); CNAP1(9918); CDKN1B(1027); IAPP(3375); ESPL1 |
| | | (9700); PDE1B(5153); HMGA2(8091); APAF1(317); KNTC1(9735); RAD52 |
| | | (5893); ZNF641(121274); LALBA(3906); FAIM2(23017); WIF1(11197); |
| | | CPM(1368); LIN7A(8825); DIP13B(55198); ANAPC7(51434); CDK2AP1 |
| | | (8099); HSMPP8(54737); FGF9(2254); BRCA2(675); KL(9365); CUL4A(8451); |
| | | CDC16(8881); FOXO1A(2308); KLF12(11278); LIG4(3981); TXNDC |
| | | (81542); PSEN1(5663); SPTLC2(9517); CHES1(1112); FGF7(2252); SCAND2 |
| | | (54581); ABHD2(11057); SLC12A6(9990); PYGO1(26108); BCL2A1(597); |
| | | GRIN2A(2903); GSPT1(2935); FTS(64400); TRADD(8717); ALOX12(239); |
| | | NLK(51701); NF1(4763); CDC6(990); EIF1(10209); CROP(51747); PRKCA |
| | | (5578); NALP1(22861); TP53(7157); GAS7(8522); BRCA1(672); C18orf1 |
| | | (753); MIB1(57534); STATIP1(55250); ROCK1(6093); TXNL1(9352); BCL2 |
| | | (596); ZNF440(126070); ZNF439(90594); ZNF440L(284390); APOE(348); |
| | | SPHK2(56848); ZNF480(147657); ZNF490(57474); ZNF100(163227); |
| | | PLCB1(23236); STK4(6789); CSE1L(1434); PARD6B(84612); RP5-1022P6.2 |
| | | (56261); UBE2V1(7335); AURKA(6790); CABLES2(81928); SON(6651); |
| | | SNF1LK(150094); BCL2L13(23786); NF2(4771); SEPT3(55964); MAPK1 |
| | | (5594); CA5B(11238); HDAC6(10013); EDA(1896); NGFRAP1(27018); BIRC4 |
| | | (331); STAG2(10735); DKC1(1736); TRAPPC2(6399); SMC1L1(8243); |
| | | CUL4B(8450); ARHGEF6(9459); |
| hsa-miR-142-3p | 35; 561; 562; | RBBP4(5928); SH3GLB1(51100); CDC14A(8556); FLVCR(28982); PROX1 |
| | 563; 564; | (5629); FRAP1(2475); TTMB(399474); SSX2IP(117178); GFI1(2672); SASS6 |
| | 2228; 2229; | (163786); DENND2D(79961); ZBTB41(360023); FH(2271); EML4(27436); |
| | | BRRN1(23397); PMS1(5378); CASP8(841); FZD7(8324); RTN4(57142); |
| | | PCAF(8850); MLH1(4292); DLEC1(9940); ACPP(55); RNF7(9616); SENP2 |
| | | (59343); FHIT(2272); STAG1(10274); LOC92689(92689); SMR3A(26952); |
| | | EREG(2069); GAB1(2549); DKK2(27123); TKTL2(84076); VEGFC(7424); |
| | | SKP2(6502); RHOBTB3(22836); APC(324); TCF7(6932); PRLR(5618); |
| | | RICTOR(253260); IRF1(3659); CDC25C(995); FAM44B(91272); PARC(23113); |
| | | PTP4A1(7803); SLC35A1(10559); QKI(9444); IER3(8870); DST(667); |
| | | ATG5(9474); FRK(2444); SGK(6446); BCLAF1(9774); MAP3K5(4217); |
| | | LATS1(9113); CREB5(9586); EGFR(1956); STAG3(10734); PMS2(5395); |
| | | HIP1(3092); WASL(8976); SLC37A3(84255); MTMR9(66036); E2F5(1875); |
| | | RIPK2(8767); NTRK2(4915); CKS2(1164); PALM2(114299); BNC2(54796); |
| | | TRIM14(9830); C10orf9(219771); ARID5B(84159); MLR2(84458); CUL2 |
| | | (8453); RAB11FTP2(22841); MKI67(4288); TEAD1(7003); ARNTL(406); |
| | | F2(2147); LRP5(4041); CUL5(8065); ATM(472); TBRG1(84897); MAP3K11 |
| | | (4296); GRM5(2915); HMGA2(8091); GAS2L3(283431); KNTC1(9735); RAN |
| | | (5901); FGF23(8074); SLC38A1(81539); SLC38A4(55089); C12orf22(81566); |
| | | DDIT3(1649); NT5DC3(51559); ZNF605(90462); FGF9(2254); TSHR |
| | | (7253); GZMB(3002); STRN3(29966); BUB1B(701); BMF(90427); FLJ11506 |
| | | (79719); P8(26471); MAPK3(5595); NF1(4763); NME1(4830); TNFRSF11A |
| | | (8792); P15RS(55197); CDC34(997); NDUFA13(51079); ATF5(22809); |
| | | TGFB1(7040); AKT1S1(84335); DUXA(503835); PLCB1(23236); PARD6B |
| | | (84612); PRKCBP1(23613); DSCR6(53820); BRWD1(54014); SNF1LK |
| | | (150094); BID(637); CA5B(11238); MLLT7(4303); AFF2(2334); SPRY3(10251); |
| | | SMC1L1(8243); WNK3(65267); SEPT6(23157); |

Table 4-19

| | | |
|---|---|---|
| hsa-miR-146a | 36; 565; 566; | HDAC1(3065); MACF1(23499); CDC14A(8556); CCNL2(81669); TNFRSF9 |
| | 567; 568; | (3604); CTNNBIP1(56998); DFFA(1676); FRAP1(2475); ST7L(54879); |
| | 569; 570; 571; | NRAS(4893); NOTCH2(4853); DEDD(9191); ASPM(259266); LPGAT1(9926); |
| | 887; 2230; | TP53BP2(7159); BIRC6(57448); PRKCE(5581); MSH2(4436); HTRA2 |
| | | (27429); RIF1(55183); PMS1(5378); CD28(940); COL4A3(1285); ANAPC1 |
| | | (64682); IL1A(3552); STAT1(6772); KLF7(8609); ERBB4(2066); HDAC4(9759); |
| | | APPL(26060); NEK11(79858); SRPRB(58477); OPA1(4976); PIK3CB |
| | | (5291); ATR(545); SIAH2(6478); LRRC15(131578); HD(3064); PAICS(10606); |
| | | CCNG2(901); NFKB1(4790); FGF2(2247); TLR2(7097); PPBP(5473); |
| | | CCNI(10983); DKK2(27123); NIPBL(25836); CARD6(84674); RHOBTB3 |
| | | (22836); APC(324); CSNK1G3(1456); RAD50(10111); BNIP1(662); PRLR |
| | | (5618); PRKAA1(5562); ATG12(9140); CDC5L(988); IL17(3605); HECA(51696); |
| | | SASH1(23328); BAK1(578); SUPT3H(8464); DST(667); TSPYL1(7259); |
| | | BCLAF1(9774); PLAGL1(5325); HDAC9(9734); ZNRF2(223082); EGFR |
| | | (1956); FZD1(8321); STAG3(10734); BCAP29(55973); CASP2(835); MAD1L1 |
| | | (8379); CDK6(1021); DLGAP2(9228); BNIP3L(665); FZD3(7976); UNC5D |
| | | (137970); TACC1(6867); SULF1(23213); PHF20L1(51105); TNFRSF10B |
| | | (8795); TNFRSF10D(8793); ADRA1A(148); SFRP1(6422); STK3(6788); |
| | | CDC37L1(55664); NTRK2(4915); DAPK1(1612); INVS(27130); ABL1 |
| | | (25); PTCH(5727); TRIM14(9830); TSC1(7248); EIF4EBP2(1979); PTEN |
| | | (5728); DOCK1(1793); PARD3(56288); SFRP5(6425); WEE1(7465); HIPK3 |
| | | (10114); RPS6KB2(6199); LOH11CR2A(4013); CHEK1(1111); EIF4G2(1982); |
| | | WT1(7490); TRAF6(7189); CDCA5(113130); BRMS1(25855); MTL5(9633); |
| | | SESN3(143686); PPP2R1B(5519); IL18(3606); CNAP1(9918); CCT2 |
| | | (10576); ING4(51147); KRAS(3845); HDAC7A(51564); FAIM2(23017); DDIT3 |
| | | (1649); DUSP6(1848); HSMPP8(54737); BRCA2(675); RB1(5925); CUL4A |
| | | (8451); BCL2L2(599); PNN(5411); TXNDC(81542); CDKN3(1033); EGLN3 |
| | | (112399); CDKL1(8814); AKT1(207); JAG2(3714); BUB1B(701); CSK |
| | | (1445); LRRK1(79705); DNAJA3(9093); CYLD(1540); PARD6A(50855); |
| | | GRIN2A(2903); ERN2(10595); MPHOSPH6(10200); PCOLN3(5119); GSG2 |
| | | (83903); ALOX15B(247); TRAF4(9618); NF1(4763); LIG3(3980); TADA2L |
| | | (6871); MAPT(4137); CROP(51747); NME1(4830); PRKCA(5578); MAP2K6 |
| | | (5608); P2RX1(5023); GAS7(8522); GAS2L2(246176); BRCA1(672); HDAC5 |
| | | (10014); MPO(4353); DCC(1630); MALT1(10892); PHLPP(23239); ESCO1 |
| | | (114799); PARD6G(84552); CHAF1A(10036); UHRF1(29128); PIK3R2 |
| | | (5296); UBA52(7311); CCNE1(898); APOE(348); HIF3A(64344); DNASE2 |
| | | (1777); PRX(57716); DEDD2(162989); ERCC2(2068); MCM8(84515); SFRS6 |
| | | (6431); STK4(6789); CSE1L(1434); RBL1(5933); SYCP2(10388); DIDO1 |
| | | (11083); SON(6651); POFUT2(23275); NF2(4771); EP300(2033); MN1 |
| | | (4330); CARD10(29775); PCTK1(5127); AGTR2(186); BIRC4(331); STAG2 |
| | | (10735); CD40LG(959); GPM6B(2824); SMC1L1(8243); BTK(695); CUL4B |
| | | (8450); CETN2(1069); |
| hsa-miR-147 | 37; 572; 573; | NBL1(4681); MACF1(23499); CDKN2C(1031); PEA15(8682); C1orf95(375057); |
| | 574; 575; | CCNL2(81669); CDC2L2(985); TNFRSF14(8764); FRAP1(2475); ZBTB17 |
| | 576; | (7709); CLSPN(63967); GFI1(2672); NOTCH2(4853); GLRX2(51022); |
| | | C1orf116(79098); TP53BP2(7159); MAPRE3(22924); CCT7(10574); KCNIP3 |
| | | (30818); BRRN1(23397); BCL2L11(10018); RIF1(55183); COL4A3 |
| | | (1285); BOK(666); YWHAQ(10971); RTN4(57142); TGFA(7039); TLK1(9874); |
| | | NCKAP1(10787); FZD5(7855); PCAF(8850); DLEC1(9940); GNAT1(2779); |
| | | ACPP(55); SENP2(59343); AXUD1(64651); NEK4(6787); PROK2(60675); |
| | | RAB6B(51560); STAG1(10274); PIK3CB(5291); ATR(545); PCGF3(10336); |
| | | DKFZP564O0823(25849); APBB2(323); BTC(685); DKK2(27123); |
| | | LEF1(51176); NIPBL(25836); CARD6(84674); MRPS30(10884); APC(324); |
| | | RAD50(10111); BNIP1(662); UNC5A(90249); PRLR(5618); BIRC1(4671); |
| | | MCC(4163); ATG12(9140); SEMA6A(57556); NRG2(9542); FGF1(2246); |
| | | CD74(972); RNF130(55819); LY86(9450); LTA(4049); IL17(3605); PTP4A1 |
| | | (7803); FOXO3A(2309); TNFAIP3(7128); SASH1(23328); C6orf85(63027); |
| | | SSR1(6745); DAXX(1616); CUL7(9820); TNFRSF21(27242); MCM3(4172); |
| | | DST(667); SESN1(27244); PLAGL1(5325); LATS1(9113); AHR(196); HDAC9 |
| | | (9734); FZD9(8326); STAG3(10734); BCAP29(55973); CUL1(8454); |
| | | MGC11257(84310); SFRP4(6424); INHBA(3624); CDK6(1021); HIPK2(28996); |
| | | TNFRSF10C(8794); FZD6(8323); MYC(4609); LZTS1(11178); UNC13B |
| | | (10497); RECK(8434); DAPK1(1612); SPIN(10927); C9orf47(286223); |
| | | IFNW1(3467); SHB(6461); KIAA0367(23273); TNFSF15(9966); TNFSF8 |
| | | (944); TRAF1(7185); CIZ1(25792); TSC1(7248); CUGBP2(10659); UNC5B( |

Table 4-20

| | | |
|---|---|---|
| | | 219699); TLX1(3195); BTRC(8945); CSPG6(9126); BAG3(9531); FLJ46831 |
| | | (399823); GPR123(84435); PARD3(56288); KCNMA1(3778); DLG5(9231); |
| | | TMEM10(93377); SMNDC1(10285); C10orf46(143384); MKI67(4288); IFITM1 |
| | | (8519); WEE1(7465); F2(2147); MS4A2(2206); SIPA1(6494); CCND1 |
| | | (595); BIRC3(330); ATM(472); IGF2(3481); RHOG(391); EIF4G2(1982); |
| | | WT1(7490); HRASLS3(11145); MAP3K11(4296); RELA(5970); CLCF1(23529); |
| | | PPP1CA(5499); FZD4(8322); SESN3(143686); PPP2R1B(5519); B3GAT1 |
| | | (27087); CNAP1(9918); PTMS(5763); MDM2(4193); APAF1(317); GAS2L3 |
| | | (283431); BCAT1(586); KRAS(3845); HDAC7A(51564); C12orf22(81566); |
| | | CDK4(1019); PCTK2(5128); DIP13B(55198); HSMPP8(54737); TNFRSF19 |
| | | (55504); BRCA2(675); RB1(5925); TFDP1(7027); CDC16(8881); LATS2 |
| | | (26524); FOXO1A(2308); PSEN1(5663); KIAA0329(9895); TRAF3(7187); |
| | | ACIN1(22985); STRN3(29966); CDKL1(8814); BMP4(652); LTBP2(4053); |
| | | TGFB3(7043); BAG5(9529); PPP1R13B(23368); AKT1(207); BUB1B |
| | | (701); MAPK6(5597); ARNT2(9915); ABHD2(11057); SLC12A6(9990); DAPK2 |
| | | (23604); CSNK1G1(53944); CALML4(91860); FLJ22795(80154); TSC2 |
| | | (7249); TRAF7(84231); DNASE1(1773); PAPD5(64282); KATNB1(10300); |
| | | NOL3(8996); CTCF(10664); KIAA0513(9764); AXIN1(8312); GSPT1(2935); |
| | | ERN2(10595); SIAH1(6477); SLC12A4(6560); TIMM22(29928); NLK |
| | | (51701); NF1(4763); PPM1D(8493); PRKCA(5578); MNT(4335); P2RX1 |
| | | (5023); SLC13A5(284111); GPS2(2874); GAS7(8522); SPAG5(10615); TCF2 |
| | | (6928); ERN1(2081); MAPRE2(10982); DCC(1630); MALT1(10892); MBP |
| | | (4155); HMG20B(10362); DNM2(1785); NDUFA13(51079); LOC148137 |
| | | (148137); CYP2S1(29785); ARHGEF1(9138); ZNF226(7769); APOE(348); |
| | | NALP2(55655); TNFSF14(8740); ELAVL1(1994); ELAVL3(1995); DEDD2 |
| | | (162989); TRIB3(57761); CEP250(11190); MYBL2(4605); BIRC7(79444); |
| | | C20orf112(140688); ELMO2(63916); CTCFL(140690); CABLES2(81928); |
| | | DIDO1(11083); SON(6651); APP(351); SNF1LK(150094); BCL2L13(23786); |
| | | ADORA2A(135); KREMEN1(83999); PGEA1(25776); SEPT3(55964); |
| | | PPARA(5465); BID(637); MAPK1(5594); PPM1F(9647); C22orf5(25829); |
| | | TOB2(10766); LDOC1L(84247); CCNB3(85417); BIRC4(331); RP6-213H19.1 |
| | | (51765); FIGF(2277); SMC1L1(8243); SEPT6(23157); ARHGEF6(9459); |
| | | FAM58A(92002); HCFC1(3054); |
| hsa-miR-149 | 38; 577; | PRKCZ(5590); NBL1(4681); SEPN1(57190); SFN(2810); KHDRBS1(10657); |
| | | HPCA(3208); MACF1(23499); NFYC(4802); PLK3(1263); RAP1A(5906); |
| | | DCLRE1B(64858); BCL9(607); BNIPL(149428); DAP3(7818); PEA15(8682); |
| | | FASLG(356); IL24(11009); TRAF5(7188); TGFB2(7042); CIAS1(114548); |
| | | TNFRSF9(3604); CTNNBIP1(56998); DFFA(1676); FRAP1(2475); CASP9 |
| | | (842); RCC2(55920); YARS(8565); CLSPN(63967); HEYL(26508); TESK2 |
| | | (10420); NOTCH2(4853); CD5L(922); DEDD(9191); ASPM(259266); |
| | | SYT2(127833); TP53BP2(7159); MAPRE3(22924); BIRC6(57448); PRKCE |
| | | (5581); CCT7(10574); BCL2L11(10018); ZAK(51776); CASP8(841); COL4A3 |
| | | (1285); PSMD1(5707); BOK(666); CARD12(58484); TMEM127(55654); |
| | | EDAR(10913); BUB1(699); IL1A(3552); IL1B(3553); BIN1(274); ERCC3 |
| | | (2071); IFTH1(64135); IGFBP5(3488); CUL3(8452); HDAC4(9759); DTYMK |
| | | (1841); NR2C2(7182); MLH1(4292); DLEC1(9940); TCTA(6988); BSN(8927); |
| | | NUDT16(131870); SMC4L1(10051); PSMD2(5708); KNG1(3827); TP73L |
| | | (8626); OPA1(4976); AXUD1(64651); TRAIP(10293); TMEM110(375346); |
| | | RAB43(339122); SIAH2(6478); HD(3064); CD38(952); IL8(3576); FGF5 |
| | | (2250); NFKB1(4790); CCNI(10983); UNC5C(8633); LEF1(51176); VEGFC |
| | | (7424); KIAA1909(153478); CCNB1(891); RAD17(5884); F2R(2149); |
| | | APC(324); RAD50(10111); ARHGAP26(23092); NDST1(3340); N4BP3(23138); |
| | | SEMA5A(9037); DAP(1611); UNG2(10309); IRF1(3659); PPP2CA(5515); |
| | | NRG2(9542); PFDN1(5201); FBXW11(23291); LOC51149(51149); RNF130 |
| | | (55819); RIPK1(8737); E2F3(1871); TNF(7124); MAPK13(5603); PARC |
| | | (23113); TTK(7272); FOXO3A(2309); TNFAIP3(7128); NEDD9(4739); |
| | | MDC1(9656); FLJ43752(401253); IHPK3(117283); MDGA1(266727); CUL7 |
| | | (9820); TNFRSF21(27242); BCLAF1(9774); LATS1(9113); IQCE(23288); |
| | | FOXK1(221937); HDAC9(9734); ADCY1(107); EGFR(1956); FZD1(8321); |
| | | STAG3(10734); C7orf19(80228); BCAP29(55973); CASP2(835); CUL1 |
| | | (8454); LOC155060(155060); TRIAD3(54476); ICA1(3382); SOSTDC1(25928); |
| | | CARD4(10392); PURB(5814); PMS2L2(5380); CDK6(1021); ZC3HC1 |
| | | (51530); SLC35B4(84912); RAI16(64760); PTK2B(2185); FZD3(7976); UNC5D |
| | | (137970); TACC1(6867); ATP6V1C1(528); LZTS1(11178); TNFRSF10B |
| | | (8795); TNFRSF10D(8793); TRIM35(23087); SFRP1(6422); EXT1(2131 |

Table 4-21

| | | |
|---|---|---|
| | | ); UNC13B(10497); NTRK2(4915); DAPK1(1612); C9orf47(286223); CKS2 |
| | | (1164); C9orf28(89853); ABL1(25); IFNW1(3467); CDKN2A(1029); BAG1 |
| | | (573); FANCG(2189); SEMA4D(10507); PTCH(5727); TNFSF8(944); TRAF1 |
| | | (7185); TSC1(7248); ANAPC2(29882); UNC5B(219699); RAI17(57178); |
| | | LZTS2(84445); BTRC(8945); PDCD11(22984); BAG3(9531); GPR26(2849); |
| | | DOCK1(1793); FRMD4A(55691); DLG5(9231); FBXW4(6468); SH3PXD2A |
| | | (9644); DCLRE1A(9937); MKI67(4288); BNIP3(664); STIM1(6786); WEE1 |
| | | (7465); HIPK3(10114); API5(8539); C11orf49(79096); INCENP(3619); |
| | | LGALS12(85329); RPS6KB2(6199); LRP5(4041); ATM(472); CD3E(916); |
| | | LOH11CR2A(4013); KCNJ5(3762); BET1L(51272); MUCDHL(53841); APBB1 |
| | | (322); WT1(7490); RAB3IL1(5866); BAD(572); MAP3K11(4296); BRMS1 |
| | | (25855); CLCF1(23529); PPP1CA(5499); FGF3(2248); SESN3(143686); |
| | | BCL9L(283149); CCND2(894); LTBR(4055); TNFRSF7(939); CDKN1B |
| | | (1027); FLJ20489(55652); TENC1(23371); ESPL1(9700); CDK2(1017); SOCS2 |
| | | (8835); APAF1(317); GAS2L3(283431); KIAA0152(9761); KNTC1(9735); |
| | | HDAC7A(51564); MCRS1(10445); CBX5(23468); DDIT3(1649); CDK4 |
| | | (1019); DUSP6(1848); PCTK2(5128); DIP13B(55198); ANAPC7(51434); |
| | | DIABLO(56616); SCARB1(949); CHFR(55743); HSMPP8(54737); TNFRSF19 |
| | | (55504); BRCA2(675); RFP2(10206); ING1(3621); CUL4A(8451); BCL2L2 |
| | | (599); RNF31(55072); PNN(5411); CGRRF1(10668); VASH1(22846); |
| | | SIVA(10572); ACIN1(22985); CIDEB(27141); EGLN3(112399); LTBP2(4053); |
| | | CHES1(1112); ITPK1(3705); PRIMA1(145270); AKT1(207); BUB1B(701); |
| | | MAPK6(5597); CORO2B(10391); KIF23(9493); IGF1R(3480); BMF(90427); |
| | | HISPPD2A(9677); FLJ38723(255180); CSNK1G1(53944); PDCD7 |
| | | (10081); STRA6(64220); BCL2A1(597); GFER(2671); TSC2(7249); TRAF7 |
| | | (84231); CCNF(899); TNFRSF12A(51330); DNASE1(1773); KIAA0350(23274); |
| | | UBPH(56061); PLK1(5347); CARD15(64127); RBL2(5934); NOL3(8996); |
| | | ELMO3(79767); CTCF(10664); CYB5B(80777); CDT1(81620); CDK10 |
| | | (8558); AXIN1(8312); PKMYT1(9088); GSPT1(2935); ERN2(10595); P8 |
| | | (26471); MAPK3(5595); SEPT1(1731); CIAPIN1(57019); TRADD(8717); RBM35B |
| | | (80004); SMPD3(55512); PHLPPL(23035); HIC1(3090); C17orf81 |
| | | (23587); TNFSF12(8742); MPDU1(9526); MAPK7(5598); NF1(4763); STARD3 |
| | | (10948); FZD2(2535); MAPT(4137); CROP(51747); TLK2(11011); PRKCA |
| | | (5578); SLC9A3R1(9368); CDK3(1018); SPHK1(8877); BIRC5(332); |
| | | CARD14(79092); BAIAP2(10458); GPS1(2873); MNT(4335); P2RX1(5023); |
| | | NALP1(22861); TP53(7157); GAS7(8522); SPAG5(10615); GAS2L2(246176); |
| | | BRCA1(672); HDAC5(10014); ERN1(2081); CASKIN2(57513); SOCS3 |
| | | (9021); TBC1D16(125058); PCYT2(5833); CABLES1(91768); MAPK4 |
| | | (5596); MALT1(10892); PHLPP(23239); FZR1(51343); UHRF1(29128); MRPL4 |
| | | (51073); DNM2(1785); MYO9B(4650); PIK3R2(5296); UBA52(7311); |
| | | CCNE1(898); MAP3K10(4294); AXL(558); ARHGEF1(9138); SPHK2(56848); |
| | | ATF5(22809); ATP8B3(148229); DAPK3(1613); SIRT2(22933); TGFB1 |
| | | (7040); PPP1R13L(10848); BBC3(27113); LIG1(3978); CARD8(22900); FUT1 |
| | | (2523); TRIB3(57761); CDC25B(994); MCM8(84515); HM13(81502); |
| | | CEP250(11190); STK4(6789); SLC9A8(23315); PARD6B(84612); BIRC7(79444); |
| | | RIMS4(140730); AURKA(6790); DIDO1(11083); USP16(10600); SON |
| | | (6651); PDXK(8566); S100B(6285); BCL2L13(23786); CDC45L(8318); HIC2 |
| | | (23119); SMARCB1(6598); ADORA2A(135); GAS2L1(10634); NF2(4771); |
| | | MTP18(51537); YWHAH(7533); GTPBP1(9567); MAP3K7IP1(10454); |
| | | EP300(2033); GTSE1(51512); HIRA(7290); MAPK1(5594); PPM1F(9647); |
| | | MN1(4330); CARD10(29775); DMC1(11144); CBX6(23466); NHP2L1(4809); |
| | | CHKB(1120); MLLT7(4303); BIRC4(331); RP6-213H19.1(51765); IKBKG |
| | | (8517); IQSEC2(23096); SMC1L1(8243); SPIN2(54466); BTK(695); SEPT6 |
| | | (23157); MTCP1(4515); |
| hsa-miR-181c | 39; 578; 579; | RCC1(1104); RBBP4(5928); MACF1(23499); NFYC(4802); SH3GLB1(51100); |
| hsa-miR-181d | 580; 581; | CDC14A(8556); RAP1A(5906); SYCP1(6847); BCL9(607); BNIPL(149428); |
| | 582; 583; 584; | CKS1B(1163); UHMK1(127933); DDR2(4921); IL24(11009); CCNL2 |
| | 585; 586; | (81669); TNFRSF14(8764); ZBTB17(7709); CLSPN(63967); FAF1(11124); |
| | 587; 40; 588; | SASS6(163786); NRAS(4893); NOTCH2(4853); GLRX2(51022); ASPM(259266); |
| | 589; | FH(2271); PPP1CB(5500); BIRC6(57448); PRKCE(5581); CCT7(10574); |
| | | HK2(3099); BRRN1(23397); BCL2L11(10018); TMEM87B(84910); RIF1 |
| | | (55183); ZAK(51776); PMS1(5378); CASP10(843); ALS2CR19(117583); |
| | | SEPT2(4735); GFPT1(2673); TIA1(7072); TGFA(7039); ZAF514(84874); |
| | | ANAPC1(64682); IL1A(3552); IL1B(3553); BIN1(274); MCM6(4175); IFIH1 |
| | | (64135); TLK1(9874); NCKAP1(10787); STAT1(6772); PGAP1(80055); |

Table 4-22

| | | |
|---|---|---|
| | | BARD1(580); CUL3(8452); PCAF(8850); PDCD6IP(10015); CTDSPL(10217); |
| | | TDGF1(6997); PCNP(57092); SMC4L1(10051); SENP2(59343); KNG1 |
| | | (3827); TP73L(8626); RASSF1(11186); NEK4(6787); STAG1(10274); TFDP2 |
| | | (7029); ATR(545); HD(3064); HCAP-G(64151); EREG(2069); SEPT11 |
| | | (55752); CCNG2(901); SPP1(6696); CCNI(10983); PDE5A(8654); NEK1(4750); |
| | | VEGFC(7424); FAM105B(90268); NIPBL(25836); ERBB2IP(55914); |
| | | CCNB1(891); TNPO1(3842); F2R(2149); CSNK1G3(1456); RAD50(10111); |
| | | UNC5A(90249); MTMR12(54545); PRLR(5618); MCC(4163); PPP2CA(5515); |
| | | CSNK1A1(1452); CD74(972); FBXW11(23291); RIPK1(8737); TNF |
| | | (7124); PARC(23113); BAG2(9532); TNFAIP3(7128); HECA(51696); SASH1 |
| | | (23328); CUL7(9820); DST(667); ATG5(9474); BCLAF1(9774); MAP3K5 |
| | | (4217); AHR(196); CREB5(9586); ZNF655(79027); CUL1(8454); TRIAD3(54476); |
| | | OSBPL3(26031); ELMO1(9844); SFRP4(6424); INHBA(3624); YWHAG |
| | | (7532); BRAF(673); DLGAP2(9228); ESCO2(157570); UNC5D(137970); |
| | | BAG4(9530); TACC1(6867); E2F5(1875); FZD6(8323); WISP1(8840); |
| | | TNFRSF10B(8795); TRIM35(23087); DUSP4(1846); RAD21(5885); TNFRSF11B |
| | | (4982); ANKRD15(23189); JAK2(3717); UNC13B(10497); RECK |
| | | (8434); DAPK1(1612); FANCG(2189); TNFSF15(9966); ZMYND11(10771); |
| | | C10orf9(219771); CDC2(983); SIRT1(23411); UNC5B(219699); PTEN(5728); |
| | | FAS(355); LZTS2(84445); BTRC(8945); SUFU(51684); CSPG6(9126); |
| | | TCF7L2(6934); CUL2(8453); DLG5(9231); FRAT2(23401); DCLRE1A(9937); |
| | | C10orf46(143384); MKI67(4288); BNIP3(664); HIPK3(10114); GPIAP1 |
| | | (4076); EHF(26298); API5(8539); EXT2(2132); INCENP(3619); VEGFB |
| | | (7423); LRP5(4041); BIRC2(329); CUL5(8065); ATM(472); MLL(4297); CHEK1 |
| | | (1111); RHOG(391); EIF4G2(1982); PTH(5741); HRASLS3(11145); PAK1 |
| | | (5058); SESN3(143686); CASP1(834); BCL9L(283149); ETS1(2113); |
| | | CDKN1B(1027); MDM2(4193); CRADD(8738); GAS2L3(283431); RAN(5901); |
| | | LRP6(4040); KRAS(3845); C12orf22(81566); WIF1(11197); PAWR(5074); |
| | | DUSP6(1848); DIP13B(55198); ANAPC7(51434); PPP1CC(5501); HSMPP8 |
| | | (54737); FGF9(2254); CDK8(1024); BRCA2(675); RB1(5925); RFP2 |
| | | (10206); FLJ40296(122183); ING1(3621); LATS2(26524); C13orf10(64062); |
| | | LIG4(3981); CDKN3(1033); MNAT1(4331); VASH1(22846); TRAF3(7187); |
| | | CIDEB(27141); LTBP2(4053); NEK9(91754); ATXN3(4287); MOAP1(64112); |
| | | BAG5(9529); GOLGA8E(390535); GOLGA8G(283768); FGF7(2252); |
| | | MAPK6(5597); LOC440295(440295); PRTG(283659); CSNK1G1(53944); |
| | | CCNF(899); DNASE1(1773); DNAJA3(9093); PAPD5(64282); CARD15(64127); |
| | | AYTL1(54947); CTCF(10664); FLJ31875(197320); NLK(51701); NF1 |
| | | (4763); TADA2L(6871); MAPT(4137); NME1(4830); TLK2(11011); PRKCA |
| | | (5578); SLC9A3R1(9368); BIRC5(332); NALP1(22861); GAS7(8522); BRCA1 |
| | | (672); MPO(4353); CBX4(8535); CSNK1D(1453); CABLES1(91768); |
| | | DCC(1630); MALT1(10892); ROCK1(6093); TXNL1(9352); BCL2(596); PARD6G |
| | | (84552); GADD45B(4616); HMG20B(10362); ZNF440(126070); ZNF439 |
| | | (90594); CCNE1(898); ZNF568(374900); MAP3K10(4294); ZNF17 |
| | | (7565); ZNF586(54807); ZNF44(51710); MGC71805(403113); ZNF443(10224); |
| | | DNASE2(1777); KIAA1559(57677); FLJ37549(163115); LOC163131 |
| | | (163131); LOC284323(284323); CARD8(22900); STK4(6789); CSE1L(1434); |
| | | PARD6B(84612); E2F1(1869); DIDO1(11083); USP16(10600); APP(351); |
| | | BRWD1(54014); TNRC6B(23112); EP300(2033); SEPT3(55964); GTSE1 |
| | | (51512); MAPK1(5594); DDX3X(1654); BIRC4(331); STAG2(10735); DKC1 |
| | | (1736); FIGF(2277); SMC1L1(8243); SEPT6(23157); FAM58A(92002); |
| hsa-miR-182* | 41; 590; 591; | H6PD(9563); BNIPL(149428); PPP1R12B(4660); TGFB2(7042); RCC2(55920); |
| | 2231; 2232; | AMIGO1(57463); C1orf198(84886); BIRC6(57448); MSH2(4436); HK2 |
| | | (3099); BRRN1(23397); G6PC2(57818); PSMD1(5707); FAM49A(81553); |
| | | EIF2AK2(5610); BUB1(699); TLK1(9874); ALS2CR15(130026); BARD1 |
| | | (580); CUL3(8452); OGG1(4968); DLEC1(9940); APPL(26060); FYCO1(79443); |
| | | TMCC1(23023); ATR(545); HD(3064); HCAP-G(64151); FGF5(2250); |
| | | CCRN4L(25819); LOC441046(441046); UNC5C(8633); ANKRD50(57182); |
| | | CASP3(836); APC(324); RAD50(10111); FLJ41603(389337); MFAP3(4238); |
| | | UBXD8(23197); PRLR(5618); PLCXD3(345557); HCN1(348980); BIRC1 |
| | | (4671); MTX3(345778); ATG12(9140); SEMA6A(57556); CSNK1A1(1452); |
| | | FBXW11(23291); HLA-A(3105); MAPK13(5603); CDC5L(988); FOXO3A |
| | | (2309); SLC16A10(117247); KIAA0319(9856); DHX16(8449); DST(667); |
| | | TMEM30A(55754); BVES(11149); FRK(2444); MAP3K5(4217); PLAGL1 |
| | | (5325); AHR(196); EGFR(1956); CASP2(835); CYCS(54205); YWHAG(7532); |
| | | BRAF(673); ESCO2(157570); UNC5D(137970); SULF1(23213); SFRP1 |

Table 4-23

| | | |
|---|---|---|
| | | (6422); FBXL6(26233); PALM2(114299); BAG1(573); TNFSF8(944); CUGBP2 |
| | | (10659); ARID5B(84159); ENTPD7(57089); DOCK1(1793); TMEM26 |
| | | (219623); MKI67(4288); TRIM44(54765); INCENP(3619); DPF2(5977); CUL5 |
| | | (8065); ATM(472); SLC1A2(6506); FLJ20625(55004); SESN3(143686); |
| | | H2AFX(3014); WNK1(65125); FLJ22028(79912); MDM2(4193); APAF1 |
| | | (317); RAN(5901); LRP6(4040); BCAT1(586); KRAS(3845); FAIM2(23017); |
| | | MMP19(4327); DUSP6(1848); HSMPP8(54737); ATP8A2(51761); BRCA2 |
| | | (675); CUL4A(8451); CSNK1A1L(122011); BCL2L2(599); PNN(5411); GPR65 |
| | | (8477); C14orf130(55148); BDKRB2(624); SALL2(6297); STRN3(29966); |
| | | LTBP2(4053); NEK9(91754); TGFB3(7043); CALML4(91860); LSM16 |
| | | (80153); UBE2I(7329); PAPD5(64282); CYLD(1540); RBL2(5934); E2F4(1874); |
| | | ERN2(10595); MAPK3(5595); TERF2(7014); RFWD3(55159); MAPK7 |
| | | (5598); NF1(4763); CDC6(990); KPNA2(3838); CARD14(79092); ACACA |
| | | (31); BRCA1(672); DCC(1630); GIPC3(126326); BCL2L12(83596); ZNF709 |
| | | (163051); GYS1(2997); STK4(6789); DNAJC5(80331); SON(6651); DSCR6 |
| | | (53820); MCM5(4174); GTPBP1(9567); MAPK1(5594); HPS4(89781); BIRC4 |
| | | (331); DKC1(1736); FIGF(2277); SYN1(6853); MTCP1(4515); DAZ2 |
| | | (57055); |
| hsa-miR-183 | 42; 592; 593; | KIF1B(23095); SESN2(83667); LCK(3932); MACF1(23499); CDC20(991); |
| | 594; 595; | SH3GLB1(51100); PROK1(84432); VANGL1(81839); UHMK1(127933); |
| | 596; 597; 598; | MDM4(4194); MAPKAPK2(9261); C1orf95(375057); TNFRSF14(8764); TNFRSF9 |
| | 599; 600; | (3604); E2F2(1870); NOTCH2(4853); THEM4(117145); MUC1(4582); |
| | 601; 602; | ASPM(259266); C1orf116(79098); ENAH(55740); ZNF496(84838); BIRC6 |
| | 603; 604; 605; | (57448); MSH2(4436); HTLF(3344); CCT7(10574); HTRA2(27429); |
| | 606; | MAL(4118); BCL2L11(10018); TMEM87B(84910); PTPN4(5775); ACVR2A |
| | | (92); RIF1(55183); CD28(940); CTDSP1(58190); SEPT2(4735); FLJ21945 |
| | | (80304); FLJ13646(79635); TGFA(7039); EDAR(10913); MGC4268(83607); |
| | | ATF2(1386); FRZB(2487); NCKAP1(10787); EPHA4(2043); PAX3(5077); |
| | | VHL(7428); CTDSPL(10217); CTNNB1(1499); ZNF662(389114); LIMD1 |
| | | (8994); GNL3(26354); ROBO2(6092); SLC25A36(55186); SMC4L1(10051); |
| | | PIK3CA(5290); KLHL24(54800); RAF1(5894); AXUD1(64651); ZNF445 |
| | | (353274); NME6(10201); WNT5A(7474); UBE1C(9039); TOMM70A(9868); |
| | | RAB6B(51560); ATR(545); SEPT11(55752); CCNG2(901); FGF5(2250); |
| | | SPP1(6696); NFKB1(4790); PLK4(10733); TMEM34(55751); DKFZp761B107 |
| | | (91050); APBB2(323); CASP3(836); FAT(2195); PDCD6(10016); NIPBL |
| | | (25836); MRPS30(10884); ERBB2IP(55914); CCNB1(891); CSNK1G3 |
| | | (1456); RAD50(10111); PHF15(23338); PCDHA9(9752); ARHGAP26(23092); |
| | | MST150(85027); MFAP3(4238); LSM11(134353); DAP(1611); PRLR(5618); |
| | | P18SRP(285672); MCC(4163); ATG12(9140); IRF1(3659); PPP2CA(5515); |
| | | KLHL3(26249); IRF4(3662); TNF(7124); LOC401252(401252); PARC |
| | | (23113); PTP4A1(7803); SH3BGRL2(83699); HECA(51696); AKAP12(9590); |
| | | QKI(9444); RP11-145H9.1(340156); HLA-DOA(3111); SUPT3H(8464); |
| | | TNFRSF21(27242); DST(667); COL12A1(1303); ATG5(9474); SESN1 |
| | | (27244); FRK(2444); TSPYL4(23270); LATS1(9113); PDCD2(5134); HDAC9 |
| | | (9734); UBE2D4(51619); EGFR(1956); TRIM24(8805); CASP2(835); PMS2 |
| | | (5395); CYCS(54205); SFRP4(6424); HIPK2(28996); VPS37A(137492); |
| | | BNIP3L(665); UNC5D(137970); TACC1(6867); UBE2V2(7336); RIPK2(8767); |
| | | FZD6(8323); SLC30A8(169026); MAL2(114569); BIN3(55909); TNFRSF10B |
| | | (8795); TRIM35(23087); DUSP4(1846); IMPAD1(54928); CCNE2 |
| | | (9134); FBXO32(114907); ANKRD15(23189); UNC13B(10497); RECK(8434); |
| | | NTRK2(4915); TRIM32(22954); TAF1L(138474); MCART1(92014); CDC14B |
| | | (8555); ANKS6(203286); ABCA1(19); ROD1(9991); TRAF1(7185); |
| | | TSC1(7248); ZMYND11(10771); WDR37(22884); CUGBP2(10659); C10orf9 |
| | | (219771); SGPL1(8879); UNC5B(219699); SFTPA1(6435); C10orf129(142827); |
| | | FRAT1(10023); SUFU(51684); PDCD4(27250); TCF7L2(6934); ITGB1 |
| | | (3688); AMID(84883); SFTPA2(6436); GPAM(57678); C10orf46(143384); |
| | | LRP5(4041); ATM(472); CBL(867); EI24(9538); EIF4G2(1982); MAP3K11 |
| | | (4296); LOC283219(283219); SESN3(143686); BCL9L(283149); FKBP4 |
| | | (2288); CNAP1(9918); IAPP(3375); ACVR1B(91); ESPL1(9700); PDE1B |
| | | (5153); CCT2(10576); NUDT4(11163); SOCS2(8835); TMPO(7112); APAF1 |
| | | (317); OAS3(4940); RAN(5901); NRIP2(83714); LRP6(4040); SLCO1A2 |
| | | (6579); MCRS1(10445); C12orf22(81566); CPM(1368); BTG1(694); PPP1CC |
| | | (5501); ANAPC5(51433); CDK2AP1(8099); ZNF605(90462); HSMPP8(54737); |
| | | SUGT1(10910); CUL4A(8451); CDC16(8881); LATS2(26524); FOXO1A |
| | | (2308); FRMD6(122786); CHX10(338917); SIVA(10572); KIAA0284( |

Table 4-24

| | | |
|---|---|---|
| | | 283638); TM9SF1(10548); DDHD1(80821); DLG7(9787); RAB15(376267); |
| | | LTBP2(4053); TTC7B(145567); BCL11B(64919); BAG5(9529); PPP1R13B |
| | | (23368); AKT1(207); CCNB2(9133); TPM1(7168); PML(5371); KIAA1024 |
| | | (23251); SLC12A6(9990); BMF(90427); CSNK1G1(53944); PDCD7(10081); |
| | | CALML4(91860); RAB40C(57799); ABAT(18); CP110(9738); PLK1(5347); |
| | | RBL2(5934); CTCF(10664); GSPT1(2935); P8(26471); SLC12A4(6560); |
| | | SMPD3(55512); EXOSC6(118460); TXNL4B(54957); TNFSF12(8742); MAPK7 |
| | | (5598); NF1(4763); FZD2(2535); MAPT(4137); CROP(51747); STXBP4 |
| | | (252983); PPM1D(8493); PRKCA(5578); KPNA2(3838); KCTD2(23510); |
| | | CDK3(1018); SPHK1(8877); BIRC5(332); P2RX1(5023); GAS7(8522); TBC1D3C |
| | | (414060); VAT1(10493); HDAC5(10014); MPO(4353); PTRH2(51651); |
| | | APPBP2(10513); ABCA8(10351); RNMT(8731); MAPK4(5596); DCC |
| | | (1630); WDR7(23335); C18orf45(85019); SMAD2(4087); HMG20B(10362); |
| | | GLT25D1(79709); HIF3A(64344); NALP2(55655); ZNF551(90233); EEF2 |
| | | (1938); EMR3(84658); OR7A5(26659); LOC284296(284296); STK4(6789); |
| | | SLC9A8(23315); C20orf177(63939); BIRC7(79444); BCL2L1(598); CABLES2 |
| | | (81928); SON(6651); DIP2A(23181); ITGB2(3689); SMARCB1(6598); |
| | | NF2(4771); LOC400924(400924); MGC16703(113691); MAPK1(5594); PPM1F |
| | | (9647); NPTXR(23467); MSL3L1(10943); TAF1(6872); OGT(8473); SLC16A2 |
| | | (6567); ARMCX4(158947); BIRC4(331); SMC1L1(8243); OPHN1 |
| | | (4983); ZDHHC15(158866); CD99L2(83692); HCFC1(3054); |
| hsa-miR-184 | 43; 607; 608; | LDLRAD2(401944); HDAC1(3065); CSF1(1435); BCL9(607); TNFRSF14 |
| | 609; 610; | (8764); DFFA(1676); CLSPN(63967); HIVEP3(59269); NOTCH2(4853); TP53BP2 |
| | 611; 612; 613; | (7159); LOC56902(56902); HK2(3099); EPB41L5(57669); CASP8(841); |
| | 614; 615; | CCT4(10575); DAG1(1605); RAF1(5894); SIAH2(6478); HD(3064); FGF10 |
| | 616; 617; | (2255); CSNK1A1(1452); CAMK2A(815); PACSIN1(29993); PARC(23113); |
| | 618; | MAP3K5(4217); IQCE(23288); EGFR(1956); ELN(2006); FZD1(8321); |
| | | CYCS(54205); INHBA(3624); PTN(5764); BAG4(9530); MYC(4609); SFRP1 |
| | | (6422); NUDT2(318); GRIN1(2902); FANCG(2189); PTCH(5727); CARD9 |
| | | (64170); LZTS2(84445); FBXW4(6468); STIM1(6786); INCENP(3619); |
| | | RPS6KB2(6199); LRP5(4041); SF1(7536); BCL9L(283149); SOCS2(8835); |
| | | MCRS1(10445); DDIT3(1649); RFP2(10206); SOX1(6656); LATS2(26524); |
| | | FOXO1A(2308); LOC283487(283487); ACIN1(22985); CSK(1445); IGF1R |
| | | (3480); NDN(4692); FLJ39743(283777); TSC2(7249); PAPD5(64282); GSG2 |
| | | (83903); RAI1(10743); CARD14(79092); NALP1(22861); SPAG5(10615); |
| | | PHB(5245); TMEM105(284186); SYDE1(85360); CRTC1(23373); FOSB |
| | | (2354); SPHK2(56848); BAX(581); SH3GL1(6455); TNPO2(30000); LIG1(3978); |
| | | SAPS1(22870); MYBL2(4605); THBD(7056); BCL2L1(598); RIMS4 |
| | | (140730); AURKA(6790); GM632(57473); SNF1LK(150094); CDC45L(8318); |
| | | MCM5(4174); EP300(2033); ACO2(50); PPARA(5465); MAPK8IP2(23542); |
| | | PDGFB(5155); MLLT7(4303); PPP2R3B(28227); |
| hsa-miR-192 | 44; 619; 620; | CDC7(8317); ATP1A2(477); SOAT1(6646); TOR1AIP1(26092); MDM4(4194); |
| hsa-miR-215 | 621; 622; | CR1(1378); C1orf107(27042); TRAF5(7188); CTNNBIP1(56998); C1orf163 |
| | 623; 624; 625; | (65260); SLC19A2(10560); C1orf24(116496); PPP1CB(5500); BRRN1 |
| | 45; 626; | (23397); LIMS1(3987); BCL2L11(10018); CFLAR(8837); FLJ13646(79635); |
| | 627; 628; 629; | IL1A(3552); ERCC3(2071); MCM6(4175); TLK1(9874); CUL3(8452); PDCD6IP |
| | 630; 631; | (10015); LIMD1(8994); APPL(26060); ACPP(55); TRAIP(10293); |
| | 632; 633; | B3GALT3(8706); WIG1(64393); HCAP-G(64151); EREG(2069); CCNG2(901); |
| | 634; 635; | NEK1(4750); NIPBL(25836); CARD6(84674); SERF1A(8293); APC(324); |
| | | CSNK1G3(1456); TCF7(6932); SGCD(6444); SMA4(11039); MCC(4163); |
| | | FBXW11(23291); ANKS1A(23294); HECA(51696); ESR1(2099); DST(667); |
| | | SYNCRIP(10492); PDCD2(5134); TMEM106B(54664); EGFR(1956); |
| | | BCAP29(55973); CYCS(54205); ELMO1(9844); ZC3HAV1(56829); UNC5D |
| | | (137970); AP3M2(10947); HOOK3(84376); UBE2V2(7336); NTRK2(4915); |
| | | C9orf47(286223); BAG1(573); STRBP(55342); C10orf9(219771); UNC5B |
| | | (219699); PCGF5(84333); ENTPD7(57089); ACADSB(36); C10orf72(196740); |
| | | CUL5(8065); ATM(472); SC5DL(6309); RNF141(50862); FZD4(8322); |
| | | PPP2R1B(5519); DDX6(1656); CCND2(894); KIAA1467(57613); MDM2 |
| | | (4193); SOCS2(8835); LRP6(4040); BCAT1(586); LIN7A(8825); FGF9(2254); |
| | | BRCA2(675); RB1(5925); RFP2(10206); DICER1(23405); FGF7(2252); |
| | | ARNT2(9915); TRAF7(84231); CLUAP1(23059); CARD15(64127); RBL2 |
| | | (5934); CTCF(10664); ERN2(10595); FTS(64400); TRADD(8717); RFWD3 |
| | | (55159); CNP(1267); NGFR(4804); CROP(51747); TLK2(11011); GPS1(2873); |
| | | PIP5K2B(8396); MALT1(10892); FOSB(2354); KCNA7(3743); KLK10 |
| | | (5655); TRIB3(57761); CSE1L(1434); GGTL3(2686); SYCP2(10388); SON( |

Table 4-25

| | | |
|---|---|---|
| | | 6651); BRWD1(54014); SNF1LK(150094); MAPK1(5594); BIRC4(331); CRSP2 |
| | | (9282); |
| hsa-miR-193a | 46; 636; 637; | TNFRSF1B(7133); PAX7(5081); SESN2(83667); KHDRBS1(10657); TXLNA |
| hsa-miR-193b | 638; 639; | (200081); LCK(3932); MACF1(23499); CDC20(991); RAP1A(5906); BCL9 |
| | 640; 641; 642; | (607); IL24(11009); HHAT(55733); TGFB2(7042); TNFRSF14(8764); CTNNBIP1 |
| | 643; 47; | (56998); FRAP1(2475); CLSPN(63967); MUTYH(4595); NOTCH2 |
| | 644; 645; 2233; | (4853); MCL1(4170); S100A6(6277); CD5L(922); TMEM63A(9725); FOSL2 |
| | | (2355); BIRC6(57448); HTRA2(27429); TCF7L1(83439); ABI2(10152); |
| | | COL4A3(1285); E2F6(1876); TIA1(7072); ANAPC1(64682); BIN1(274); NCKAP1 |
| | | (10787); TNS1(7145); HDAC4(9759); PDCD6IP(10015); TDGF1(6997); |
| | | MCM2(4171); RNF7(9616); TADA3L(10474); AXUD1(64651); TRAIP |
| | | (10293); RASSF1(11186); TUSC4(10641); IL17RD(54756); FHIT(2272); |
| | | ATR(545); HD(3064); ANAPC4(29945); CCNG2(901); SPP1(6696); NFKB1 |
| | | (4790); DKK2(27123); ERBB2IP(55914); APC(324); PHF15(23338); UNC5A |
| | | (90249); ZNF346(23567); N4BP3(23138); BIRC1(4671); MCC(4163); IRF1 |
| | | (3659); AFF4(27125); HDAC3(8841); FGF1(2246); PARC(23113); BAG2 |
| | | (9532); HECA(51696); NEDD9(4739); BAK1(578); CUL7(9820); TNFRSF21 |
| | | (27242); DST(667); CCNC(892); PDCD2(5134); BCAP29(55973); SOSTDC1 |
| | | (25928); CDK6(1021); TNFRSF10C(8794); FZD3(7976); RIPK2(8767); |
| | | WISP1(8840); LZTS1(11178); BIN3(55909); TNFRSF10B(8795); TP53INP1 |
| | | (94241); MTSS1(9788); ANKRD15(23189); CDC37L1(55664); UNC13B |
| | | (10497); OLFML2A(169611); ABL1(25); TRAF2(7186); BAG1(573); ZBTB5 |
| | | (9925); GAS1(2619); TRIM14(9830); TRAF1(7185); TSC1(7248); C10orf9 |
| | | (219771); UNC5B(219699); PTEN(5728); LZTS2(84445); BTRC(8945); |
| | | SUFU(51684); TCF7L2(6934); BAG3(9531); DOCK1(1793); DLG5(9231); |
| | | C10orf46(143384); BNIP3(664); HIPK3(10114); SIPA1(6494); RPS6KB2 |
| | | (6199); LRP5(4041); CCND1(595); ATM(472); CHEK1(1111); HRAS(3265); |
| | | WT1(7490); CLCF1(23529); PPP1CA(5499); FGF3(2248); CNAP1(9918); |
| | | PTPN6(5777); ESPL1(9700); MDM2(4193); SOCS2(8835); APAF1(317); |
| | | KRAS(3845); MCRS1(10445); FAIM2(23017); C12orf22(81566); BRCA2 |
| | | (675); ING1(3621); BCL2L2(599); PSEN1(5663); VASH1(22846); EGLN3(112399); |
| | | LTBP2(4053); JAG2(3714); SNX1(6642); IGF1R(3480); BMF(90427); |
| | | BCL2L10(10017); DAPK2(23604); CSNK1G1(53944); RAB40C(57799); |
| | | TSC2(7249); RBL2(5934); GNAO1(2775); KATNB1(10300); PARD6A(50855); |
| | | CDT1(81620); CDK10(8558); AXIN1(8312); PKMYT1(9088); MAPK3 |
| | | (5595); SEPT1(1731); SIAH1(6477); CIAPIN1(57019); HIC1(3090); GSG2 |
| | | (83903); NTN1(9423); NF1(4763); TADA2L(6871); NGFR(4804); PRKCA |
| | | (5578); SLC9A3R1(9368); CARD14(79092); ANAPC11(51529); MNT(4335); |
| | | GAS7(8522); BRCA1(672); MPO(4353); SOCS3(9021); CSNK1D(1453); |
| | | DCC(1630); PHLPP(23239); DOK6(220164); APC2(10297); FZR1(51343); |
| | | TNFSF9(8744); DNM2(1785); PIK3R2(5296); UBA52(7311); ATF5(22809); |
| | | AES(166); DAPK3(1613); CDC37(11140); KLK10(5655); SNPH(9751); CEP250 |
| | | (11190); BCL2L1(598); E2F1(1869); CTCFL(140690); DIDO1(11083); |
| | | SON(6651); CHAF1B(8208); BCL2L13(23786); CDC45L(8318); SEPT5 |
| | | (5413); NF2(4771); PGEA1(25776); EP300(2033); SEPT3(55964); GTSE1 |
| | | (51512); MAPK8IP2(23542); BID(637); MAPK1(5594); PPM1F(9647); MN1 |
| | | (4330); CHEK2(11200); TOB2(10766); HDAC6(10013); MLLT7(4303); STAG2 |
| | | (10735); IKBKG(8517); FIGF(2277); SMC1L1(8243); BTK(695); SEPT6 |
| | | (23157); ARHGEF6(9459); HCFC1(3054); |
| hsa-miR-195 | 48; 646; 647; | PRKCZ(5590); DFFB(1677); KIF1B(23095); CDC42(998); NUDC(10726); |
| hsa-miR-424 | 648; 649; | WDTC1(23038); SESN2(83667); HDAC1(3065); MACF1(23499); PLK3(1263); |
| hsa-miR-497 | 650; 49; 50; | CDC14A(8556); RAP1A(5906); BCL9(607); C1orf60(65123); DAP3(7818); |
| | 651; 652; 653; | UHMK1(127933); ADORA1(134); MDM4(4194); G0S2(50486); CIAS1 |
| | 888; 889; | (114548); TNFRSF18(8784); CDC2L2(985); TNFRSF14(8764); CTNNBIP1 |
| | 890; 891; | (56998); LUZP1(7798); YARS(8565); CLSPN(63967); GFI1(2672); NOTCH2 |
| | | (4853); MCL1(4170); PYGO2(90780); DEDD(9191); ASPM(259266); |
| | | NUCKS1(64710); TP53BP2(7159); FH(2271); MAPRE3(22924); BIRC6(57448); |
| | | PRKCE(5581); HTRA2(27429); HK2(3099); KCNIP3(30818); BRRN1 |
| | | (23397); BCL2L11(10018); PLEKHB2(55041); RIF1(55183); TAIP-2(80034); |
| | | ZAK(51776); CFLAR(8837); CD28(940); NRP2(8828); INHA(3623); |
| | | COL4A3(1285); ASB1(51665); SEPT2(4735); BOK(666); ATG4B(23192); ING5 |
| | | (84289); YWHAQ(10971); CGREF1(10669); CARD12(58484); RTN4 |
| | | (57142); EDAR(10913); ANAPC1(64682); IL1A(3552); ERCC3(2071); MCM6 |
| | | (4175); IFIH1(64135); TLK1(9874); FRZB(2487); FZD5(7855); BARD1 |
| | | (580); TUBA1(7277); CUL3(8452); IL17RE(132014); PDCD6IP(10015); CT |

Table 4-26

NNB1(1499); LIMD1(8994); TDGF1(6997); RBM5(10181); GNL3(26354);
NEK11(79858); SRPRB(58477); FAIM(55179); FXR1(8087); PSMD2(5708);
SENP2(59343); KNG1(3827); RAF1(5894); AXUD1(64651); IHPK1(9807);
DNASE1L3(1776); PROK2(60675); CD80(941); GSK3B(2932); TMCC1
(23023); STAG1(10274); ATR(545); PLSCR4(57088); MASP1(5648); WHSC1
(7468); HD(3064); EREG(2069); SEPT11(55752); CCNG2(901); NFKB1
(4790); FGF2(2247); PLK4(10733); APBB2(323); PPBP(5473); RASGEF1B
(153020); SNCA(6622); UNC5C(8633); LEF1(51176); SFRP2(6423); NEK1
(4750); NIPBL(25836); FLJ37543(285668); ERBB2IP(55914); F2R(2149);
APC(324); CSNK1G3(1456); RAD50(10111); UNC5A(90249); PRLR(5618);
GDNF(2668); PRKAA1(5562); FGF10(2255); ATG12(9140); SEMA6A
(57556); IRF1(3659); PPP2CA(5515); NME5(8382); CDC23(8697); NRG2
(9542); CD14(929); FBXW11(23291); DUSP1(1843); DDX41(51428); E2F3
(1871); LTA(4049); CSNK2B(1460); MAPK13(5603); CDKN1A(1026); PARC
(23113); VEGF(7422); CDC5L(988); IL17(3605); PTP4A1(7803); TNFAIP3
(7128); GRM1(2911); TFAP2A(7020); NEDD9(4739); DHX16(8449); IER3
(8870); IHPK3(117283); CCND3(896); CUL7(9820); TNFRSF21(27242);
DST(667); CCNC(892); SESN1(27244); SGK(6446); MAP3K5(4217); LATS1
(9113); FOXK1(221937); SEPT7(989); STK17A(9263); EGFR(1956); FZD9
(8326); LOC441257(441257); FZD1(8321); STAG3(10734); RINT1(60561);
BCAP29(55973); MAD1L1(8379); TRIAD3(54476); CYCS(54205); CARD4
(10392); IGFBP3(3486); CDK6(1021); MCM7(4176); FIS1(51024); FASTK
(10922); POLR3D(661); BNIP3L(665); PTK2B(2185); UNC5D(137970);
BAG4(9530); TACC1(6867); E2F5(1875); FZD6(8323); MYC(4609); WISP1
(8840); TNFRSF10B(8795); TNFRSF10D(8793); ADRA1A(148); TRIM35
(23087); CLU(1191); DUSP4(1846); FGFR1(2260); SFRP1(6422); CCNE2
(9134); TP53INP1(94241); MTSS1(9788); ANKRD15(23189); CDC37L1
(55664); JAK2(3717); UNC13B(10497); RECK(8434); NTRK2(4915); DAPK1
(1612); GADD45G(10912); INVS(27130); C9orf91(203197); CDK9(1025);
FREQ(23413); ABL1(25); TRAF2(7186); IFNA2(3440); CDKN2A(1029);
CDKN2B(1030); TAF1L(138474); GAS1(2619); SEMA4D(10507); PTCH
(5727); TRIM14(9830); CTNNAL1(8727); PHF19(26147); TRAF1(7185);
PPP6C(5537); TSC1(7248); ZMYND11(10771); C10orf9(219771); SGPL1
(8879); UNC5B(219699); C10orf42(90550); FRAT1(10023); LZTS2(84445);
BTRC(8945); SUFU(51684); PDCD11(22984); CSPG6(9126); TCF7L2
(6934); CUL2(8453); FZD8(8325); C10orf54(64115); PPP3CB(5532); KCNMA1
(3778); FRAT2(23401); SFRP5(6425); HPSE2(60495); FBXW4(6468);
SMNDC1(10285); DCLRE1A(9937); C10orf46(143384); TIAL1(7073); MKI67
(4288); BNIP3(664); STIM1(6786); WEE1(7465); EXT2(2132); KIAA0652
(9776); INCENP(3619); LGALS12(85329); VEGFB(7423); ARL2(402);
RPS6KB2(6199); LRP5(4041); CCND1(595); FADD(8772); ATM(472); CD3E
(916); LOH11CR2A(4013); CHEK1(1111); IGF2(3481); CDKN1C(1028);
DKK3(27122); PTH(5741); WT1(7490); ZNF289(84364); CYBASC3(220002);
HRASLS3(11145); MAP3K11(4296); BRMS1(25855); PPP1CA(5499);
PAK1(5058); FZD4(8322); SESN3(143686); CASP4(837); CASP5(838);
CASP1(834); PPP2R1B(5519); ZW10(9183); BCL9L(283149); H2AFX(3014);
CCND2(894); LTBR(4055); TNFRSF7(939); CNAP1(9918); ESPL1(9700);
PDE1B(5153); PA2G4(5036); MDM2(4193); CCT2(10576); GAS2L3(283431);
FZD10(11211); TNFRSF1A(7132); NOL1(4839); LRP6(4040); KRAS
(3845); HDAC7A(51564); FAIM2(23017); C12orf22(81566); CDK4(1019);
WIF1(11197); PAWR(5074); DUSP6(1848); PCTK2(5128); DIP13B(55198);
HRK(8739); FBXO21(23014); ANAPC5(51433); MPHOSPH9(10198); SCARB1
(949); CHFR(55743); FGF9(2254); SUGT1(10910); CUL4A(8451);
LATS2(26524); FOXO1A(2308); LIG4(3981); BCL2L2(599); KIAA0247(9766);
PSEN1(5663); TRAF3(7187); CIDEB(27141); LTBP2(4053); KIAA0317
(9870); BAG5(9529); PPP1R13B(23368); JAG2(3714); FGF7(2252); MAPK6
(5597); FAM63B(54629); KIF23(9493); ABHD2(11057); IGF1R(3480);
AVEN(57099); SLC12A6(9990); BMF(90427); CSNK1G1(53944); CALML4
(91860); TSC2(7249); CCNF(899); KREMEN2(79412); DNASE1(1773);
PLK1(5347); CARD15(64127); RBL2(5934); KATNB1(10300); NOL3(8996);
ELMO3(79767); NFATC3(4775); CYB5B(80777); CDT1(81620); CDK10
(8558); ERN2(10595); MAPK3(5595); SIAH1(6477); FTS(64400); CIAPIN1
(57019); APPBP1(8883); SLC12A4(6560); PCOLN3(5119); HIC1(3090);
PAFAH1B1(5048); ALOX12(239); ALOX15B(247); NTN1(9423); MAPK7
(5598); NLK(51701); TRAF4(9618); NF1(4763); CCL2(6347); LIG3(3980);

Table 4-27

| | | |
|---|---|---|
| | | CDC6(990); FZD2(2535); MAPT(4137); NGFR(4804); CROP(51747); PPM1D |
| | | (8493); PRKCA(5578); SPHK1(8877); BIRC5(332); CARD14(79092); SCARF1 |
| | | (8578); MNT(4335); KIAA0664(23277); P2RX1(5023); NALP1(22861); |
| | | GAS7(8522); SPAG5(10615); BECN1(8678); BRCA1(672); GFAP(2670); |
| | | PHB(5245); MPO(4353); PTRH2(51651); ERN1(2081); SOCS3(9021); CBX4 |
| | | (8535); TBC1D16(125058); ARHGDIA(396); CSNK1D(1453); C18orf1 |
| | | (753); PHLPP(23239); SERPINB2(5055); ROCK1(6093); ESCO1(114799); |
| | | BCL2(596); CDC34(997); CSNK1G2(1455); FZR1(51343); TNFSF9(8744); |
| | | ILF3(3609); PIK3R2(5296); UBA52(7311); CCNE1(898); APLP1(333); |
| | | MAP3K10(4294); AXL(558); ARHGEF1(9138); BCL3(602); APOE(348); PPP2R1A |
| | | (5518); NALP2(55655); AURKC(6795); C19orf6(91304); MATK(4145); |
| | | DAPK3(1613); TNFSF7(970); INSR(3643); CDKN2D(1032); DNASE2 |
| | | (1777); CYP4F11(57834); ELL(8178); YIF1B(90522); CAPN12(147968); |
| | | TGFB1(7040); DMPK(1760); LIG1(3978); CGB7(94027); KLK10(5655); CDC25B |
| | | (994); MCM8(84515); MAPRE1(22919); CEP250(11190); TGIF2(60436); |
| | | MYBL2(4605); C20orf121(79183); STK4(6789); UBE2C(11065); CD40 |
| | | (958); CSE1L(1434); CSNK2A1(1457); E2F1(1869); RBL1(5933); ELMO2 |
| | | (63916); UBE2V1(7335); CTCFL(140690); CABLES2(81928); DIDO1(11083); |
| | | BTBD4(140685); USP16(10600); SON(6651); APP(351); SNF1LK(150094); |
| | | BCL2L13(23786); SEPT5(5413); CRKL(1399); ADORA2A(135); GAS2L1 |
| | | (10634); NF2(4771); YWHAH(7533); TNRC6B(23112); EP300(2033); |
| | | SEPT3(55964); BIK(638); GTSE1(51512); BID(637); HTF9C(27037); MAPK1 |
| | | (5594); PPM1F(9647); MN1(4330); CHEK2(11200); CARD10(29775); |
| | | PDGFB(5155); TOB2(10766); NHP2L1(4809); SMC1L2(27127); PCTK1 |
| | | (5127); HDAC6(10013); EDA(1896); TAF1(6872); AGTR2(186); CXorf6(10046); |
| | | FIGF(2277); BTK(695); CUL4B(8450); APLN(8862); ARHGEF6(9459); |
| | | FAM58A(92002); BCAP31(10134); MTCP1(4515); |
| hsa-miR-196a | 51; 654; 655; | PRKCZ(5590); TP73(7161); SESN2(83667); LCK(3932); RBBP4(5928); PLK3 |
| hsa-miR-196b | 656; 657; | (1263); RAVER2(55225); RAP1A(5906); LIX1L(128077); BCL9(607); PBX1 |
| | 658; 659; 660; | (5087); CIAS1(114548); DFFA(1676); FRAP1(2475); RCC2(55920); E2F2 |
| | 661; 662; | (1870); BSDC1(55108); TRIM62(55223); CLSPN(63967); SPATA6(54558); |
| | 663; 664; | NRAS(4893); NOTCH2(4853); C1orf25(81627); ASPM(259266); PPP1R15B |
| | 665; 666; 667; | (84919); RHOB(388); PPP1CB(5500); BIRC6(57448); MSH2(4436); |
| | 668; 52; | MAL(4118); KCNIP3(30818); BCL2L11(10018); EPC2(26122); KLHL23(151230); |
| | 669; 670; 671; | ZAK(51776); EIF2AK2(5610); RGPD5(84220); ARL5A(26225); IFIH1 |
| | 672; 673; | (64135); CAPN7(23473); OPA1(4976); RAF1(5894); ATR(545); IFT80(57560); |
| | 674; 675; | ANAPC4(29945); NFKB1(4790); FGF2(2247); RG9MTD2(93587); |
| | 2234; | TRPC3(7222); SKP2(6502); NIPBL(25836); LOC285636(285636); F2R(2149); |
| | | DCP2(167227); CSNK1G3(1456); TCF7(6932); PRLR(5618); ACSL6 |
| | | (23305); CDC25C(995); DDX41(51428); LIN28B(389421); CCND3(896); DST |
| | | (667); PDCD2(5134); HDAC9(9734); SEPT7(989); EGFR(1956); BCAP29 |
| | | (55973); PMS2(5395); HOXA7(3204); HOXA9(3205); CARD4(10392); TMED4 |
| | | (222068); YWHAG(7532); FASTK(10922); POLR3D(661); PTK2B(2185); |
| | | FZD3(7976); TERF1(7013); TNFRSF10D(8793); CLU(1191); TP53INP1 |
| | | (94241); EXT1(2131); FBXL6(26233); DAPK1(1612); PAPPA(5069); ABL1 |
| | | (25); CDKN2B(1030); BAG1(573); CIZ1(25792); TSC1(7248); ZMYND11 |
| | | (10771); MRC1L1(414308); C10orf9(219771); PRKG1(5592); FAS(355); |
| | | CCNJ(54619); MLR2(84458); PDCD11(22984); CSPG6(9126); ATRNL1(26033); |
| | | DOCK1(1793); PARD3(56288); DLG5(9231); DCLRE1A(9937); TUB |
| | | (7275); LRP5(4041); CCND1(595); BIRC3(330); TBRG1(84897); MAP3K11 |
| | | (4296); CLCF1(23529); PPP1CA(5499); FGF4(2249); PAK1(5058); SESN3 |
| | | (143686); PPP2R1B(5519); LTBR(4055); BCL2L14(79370); CDKN1B |
| | | (1027); LARP4(113251); HOXC8(3224); HMGA2(8091); CCT2(10576); SOCS2 |
| | | (8835); APAF1(317); FZD10(11211); LRP6(4040); BCAT1(586); KIAA0286 |
| | | (23306); PAWR(5074); EEA1(8411); PPP1CC(5501); FBXO21(23014); |
| | | TNFRSF19(55504); CDK8(1024); RFP2(10206); LOC440138(440138); ING1 |
| | | (3621); LATS2(26524); FOXO1A(2308); NEK3(4752); C13orf10(64062); |
| | | PNN(5411); TXNDC(81542); CALM1(801); NFKBIA(4792); DLG7(9787); |
| | | LTBP2(4053); AKT1(207); ZNF609(23060); CSK(1445); RASGRP1(10125); |
| | | BCL2L10(10017); PYGO1(26108); PRTG(283659); DAPK2(23604); TSC2 |
| | | (7249); PLK1(5347); CYLD(1540); GAN(8139); MAPK3(5595); ALOX12(239); |
| | | TNFSF12(8742); ALOX15B(247); NTN1(9423); CPD(1362); NF1(4763); |
| | | IGF2BP1(10642); KPNA2(3838); GPS1(2873); AURKB(9212); GAS7 |
| | | (8522); MPO(4353); LIMD2(80774); PHLPP(23239); ROCK1(6093); AQP4 |
| | | (361); ZNF24(7572); CDC34(997); CSNK1G2(1455); MAP3K10(4294); PPP |

Table 4-28

| | | |
|---|---|---|
| | | 2R1A(5518); DAPK3(1613); DEDD2(162989); ERCC2(2068); OPA3(80207); |
| | | DMWD(1762); LIG1(3978); MCM8(84515); PARD6B(84612); CSNK2A1 |
| | | (1457); C20orf54(113278); SIRPB1(10326); ELMO2(63916); AURKA(6790); |
| | | SYCP2(10388); CABLES2(81928); BACH1(571); S100B(6285); BCL2L13 |
| | | (23786); SMARCB1(6598); GTSE1(51512); MAPK1(5594); PPM1F(9647); |
| | | HDAC6(10013); SLC9A6(10479); PPP2R3B(28227); SMC1L1(8243); |
| | | WNK3(65267); RNF12(51132); BTK(695); CUL4B(8450); FAM58A(92002); |
| | | HCFC1(3054); MECP2(4204); DAZ2(57055); |
| hsa-miR-197 | 53; 676; 677; | PRKCZ(5590); TNFRSF1B(7133); NUDC(10726); SESN2(83667); LCK(3932); |
| | 678; 679; | MACF1(23499); BNIPL(149428); CKS1B(1163); UHMK1(127933); ADORA1 |
| | 680; 681; | (134); IL24(11009); TGFB2(7042); CDC2L2(985); CTNNBIP1(56998); |
| | | MAD2L2(10459); FBXO42(54455); E2F2(1870); YARS(8565); PYGO2 |
| | | (90780); GLRX2(51022); ASPM(259266); SOX11(6664); RHOB(388); BIRC6 |
| | | (57448); HK2(3099); KCNIP3(30818); RIF1(55183); CFLAR(8837); CASP10 |
| | | (843); CASP8(841); COL4A3(1285); PSMD1(5707); BOK(666); YWHAQ |
| | | (10971); EDAR(10913); SEPT10(151011); ANAPC1(64682); IFIH1(64135); |
| | | TLK1(9874); STAT1(6772); STK17B(9262); PAX3(5077); MLH1(4292); |
| | | TDGF1(6997); RBM5(10181); MCM2(4171); NUDT16(131870); SENP2 |
| | | (59343); KNG1(3827); TP73L(8626); CIDEC(63924); GSK3B(2932); STAG1 |
| | | (10274); ATR(545); TNFSF10(8743); SH3BP2(6452); CD38(952); SEPT11 |
| | | (55752); CCNG2(901); SNCA(6622); UNC5C(8633); MAD2L1(4085); NEK1 |
| | | (4750); CASP3(836); FAM105B(90268); MRPS30(10884); RAD17(5884); |
| | | RAD50(10111); UNC5A(90249); BIRC1(4671); SEMA6A(57556); RIPK1 |
| | | (8737); MAPK13(5603); BAG2(9532); TTK(7272); FOXO3A(2309); HECA |
| | | (51696); RP11-145H9.1(340156); DHX16(8449); MDC1(9656); IER3(8870); |
| | | TNFRSF21(27242); SGK(6446); DEADC1(134637); FOXK1(221937); AHR |
| | | (196); STK17A(9263); EGFR(1956); STAG3(10734); RINT1(60561); BCAP29 |
| | | (55973); ELMO1(9844); INHBA(3624); IGFBP3(3486); CDK6(1021); |
| | | BRAF(673); TNFRSF10C(8794); PTK2B(2185); FZD3(7976); SULF1(23213); |
| | | FZD6(8323); TNFRSF10B(8795); TRIM35(23087); MTSS1(9788); JAK2 |
| | | (3717); UNC13B(10497); DAPK1(1612); C9orf47(286223); GADD45G |
| | | (10912); CDK9(1025); TRAF2(7186); TAF1L(138474); BAG1(573); GAS1(2619); |
| | | SEMA4D(10507); PTCH(5727); ANAPC2(29882); ZMYND11(10771); |
| | | SIRT1(23411); SGPL1(8879); UNC5B(219699); PTEN(5728); KIF11(3832); |
| | | NOLC1(9221); PDCD11(22984); CSPG6(9126); DOCK1(1793); PPP3CB |
| | | (5532); DLG5(9231); DCLRE1A(9937); MKI67(4288); WEE1(7465); HIPK3 |
| | | (10114); API5(8539); F2(2147); SSSCA1(10534); SIPA1(6494); EI24(9538); |
| | | FGF4(2249); PAK1(5058); GAB2(9846); PPP2R1B(5519); IL18(3606); |
| | | BCL9L(283149); CCND2(894); PTPN6(5777); BCL2L14(79370); IAPP(3375); |
| | | DDX11(1663); CDK2(1017); CCT2(10576); KNTC1(9735); RAN(5901); |
| | | FGF6(2251); ING4(51147); C12orf22(81566); ANAPC7(51434); PPP1CC |
| | | (5501); ANAPC5(51433); FGF9(2254); TNFRSF19(55504); CDK8(1024); LIG4 |
| | | (3981); BCL2L2(599); VASH1(22846); NEK9(91754); TGFB3(7043); PPP1R13B |
| | | (23368); MAPK6(5597); CCNB2(9133); IGF1R(3480); SLC12A6 |
| | | (9990); BMF(90427); BCL2L10(10017); PYGO1(26108); TSC2(7249); TRAF7 |
| | | (84231); DNAJA3(9093); CARD15(64127); CYLD(1540); KATNB1(10300); |
| | | E2F4(1874); ELMO3(79767); MAPK3(5595); SEPT1(1731); DNAJA2 |
| | | (10294); CSNK2A2(1459); SLC12A4(6560); SMPD3(55512); NTN1(9423); |
| | | MAPK7(5598); NLK(51701); TRAF4(9618); CDC6(990); MAPT(4137); NGFR |
| | | (4804); NME1(4830); PPM1D(8493); PRKCA(5578); KPNA2(3838); SLC9A3R1 |
| | | (9368); CDK3(1018); SPHK1(8877); FLJ35220(284131); MNT(4335); |
| | | NALP1(22861); SPAG5(10615); BRCA1(672); HDAC5(10014); PHB(5245); |
| | | SEPT4(5414); ERN1(2081); SOCS3(9021); FLJ46026(400627); MAPK4 |
| | | (5596); DCC(1630); ROCK1(6093); TXNL4A(10907); HMG20B(10362); |
| | | DNM2(1785); LRP3(4037); BCL2L12(83596); DAPK3(1613); LIG1(3978); |
| | | KLK10(5655); MCM8(84515); PLCB1(23236); CEP250(11190); CD40(958); |
| | | E2F1(1869); RBL1(5933); ELMO2(63916); DIDO1(11083); USP16(10600); |
| | | SON(6651); MX1(4599); SNF1LK(150094); BCL2L13(23786); LOC400891 |
| | | (400891); NF2(4771); SYNGR1(9145); EP300(2033); MAPK1(5594); PPM1F |
| | | (9647); SSTR3(6753); DMC1(11144); MAPK12(6300); PCTK1(5127); |
| | | TAF1(6872); NGFRAP1(27018); BIRC4(331); STAG2(10735); ZIC3(7547); |
| | | DKC1(1736); PPP2R3B(28227); FIGF(2277); SMC1L1(8243); SEPT6(23157); |
| | | ARHGEF6(9459); HCFC1(3054); |
| hsa-miR-199a* | 54; 682; 683; | CDC42(998); NUDC(10726); KHDRBS1(10657); MACF1(23499); SH3GLB1 |
| | 2235; | (51100); CDC14A(8556); SYCP1(6847); UHMK1(127933); IL19(29949) |

Table 4-29

| | | |
|---|---|---|
| | | ; TGFB2(7042); CIAS1(114548); FRAP1(2475); RCC2(55920); ZNF436(80818); |
| | | E2F2(1870); SASS6(163786); NOTCH2(4853); MCL1(4170); RGS5(8490); |
| | | AKT3(10000); PRKCE(5581); HTRA2(27429); BCL2L11(10018); RIF1 |
| | | (55183); INHA(3623); MCM6(4175); NCKAP1(10787); ERBB4(2066); PAX3 |
| | | (5077); CUL3(8452); CHL1(10752); CTNNB1(1499); RBM5(10181); ACPP |
| | | (55); RAP2B(5912); FXR1(8087); KLHL24(54800); KNG1(3827); TP73L |
| | | (8626); OPA1(4976); TADA3L(10474); CLASP2(23122); NME6(10201); |
| | | TRAIP(10293); PIK3CB(5291); ATR(545); SIAH2(6478); HD(3064); CCNG2 |
| | | (901); FGF2(2247); UNC5C(8633); CARD6(84674); ERBB2IP(55914); |
| | | CCNB1(891); MCC(4163); IRF1(3659); NME5(8382); CDC25C(995); HDAC3 |
| | | (8841); FGF1(2246); CSNK1A1(1452); FBXW11(23291); LY86(9450); PARC |
| | | (23113); VEGF(7422); PTP4A1(7803); PHACTR2(9749); QKI(9444); IER3 |
| | | (8870); CCND3(896); CUL7(9820); MCM3(4172); DST(667); CCNC(892); |
| | | ECHDC1(55862); MAP3K5(4217); LATS1(9113); HDAC9(9734); BCAP29 |
| | | (55973); CUL1(8454); TRIAD3(54476); CYCS(54205); HGF(3082); CDK6 |
| | | (1021); ESCO2(157570); BAG4(9530); RIPK2(8767); FZD6(8323); WISP1 |
| | | (8840); TNFRSF10D(8793); DKK4(27121); RECK(8434); BAG1(573); PTCH |
| | | (5727); SGPL1(8879); FAS(355); NOLC1(9221); SUFU(51684); DCLRE1A |
| | | (9937); C10orf46(143384); BNIP3(664); HIPK3(10114); EXT2(2132); |
| | | INCENP(3619); LGALS12(85329); DPF2(5977); ATM(472); CBL(867); LOH11CR2A |
| | | (4013); RNF141(50862); DKK3(27122); WT1(7490); RELA(5970); |
| | | PPP1CA(5499); PPP2R1B(5519); ZW10(9183); CNAP1(9918); BCL2L14 |
| | | (79370); SOCS2(8835); NOL1(4839); BCAT1(586); SLC38A1(81539); PAWR |
| | | (5074); BTG1(694); PCTK2(5128); DIP13B(55198); MGC15619(84329); |
| | | ANAPC5(51433); MPHOSPH9(10198); SCARB1(949); BRCA2(675); RFP2 |
| | | (10206); CDC16(8881); CSNK1A1L(122011); GPR65(8477); TRAF3(7187); |
| | | CDKL1(8814); JAG2(3714); DNAJA4(55466); CSNK1G1(53944); DNAJA3 |
| | | (9093); CIITA(4261); CYLD(1540); RBL2(5934); GSPT1(2935); TXNL4B |
| | | (54957); PAFAH1B1(5048); TRAF4(9618); CROP(51747); NME1(4830); |
| | | TLK2(11011); MAP2K6(5608); BIRC5(332); MNT(4335); SPAG5(10615); |
| | | GAS2L2(246176); BRCA1(672); HDAC5(10014); MAPRE2(10982); NDUFA13 |
| | | (51079); AXL(558); DPF1(8193); TGFB1(7040); DEDD2(162989); |
| | | CARD8(22900); UBE2C(11065); RBL1(5933); CTCFL(140690); SON(6651); |
| | | MX1(4599); BCL2L13(23786); SEPT3(55964); HDAC6(10013); STAG2 |
| | | (10735); TAB3(257397); CUL4B(8450); HCFC1(3054); |
| hsa-miR-200a | 55; 684; 685; | DFFB(1677); CLCN6(1185); SESN2(83667); MACF1(23499); PRKACB(5567); |
| | 686; 687; | SH3GLB1(51100); HS2ST1(9653); CDC14A(8556); PROK1(84432); |
| | 688; 689; 690; | BCL9(607); BNIPL(149428); PEA15(8682); LY9(4063); UHMK1(127933); |
| | 691; 692; | FASLG(356); RALGPS2(55103); C1orf21(81563); PPP1R12B(4660); ADORA1 |
| | 892; 893; | (134); MDM4(4194); TRAF5(7188); TGFB2(7042); C1orf69(200205); |
| | 894; 895; 896; | CIAS1(114548); E2F2(1870); CLSPN(63967); TESK2(10420); FAF1(11124); |
| | | SLC35D1(23169); SYT6(148281); NOTCH2(4853); SELE(6401); ARPC5 |
| | | (10092); GLRX2(51022); ASPM(259266); SLC30A1(7779); PCNXL2(80003); |
| | | SELI(85465); BIRC6(57448); PRKCE(5581); PROC(5624); LOC339745 |
| | | (339745); MGC52057(130574); ITGA6(3655); ZAK(51776); PMS1(5378); |
| | | SLC39A10(57181); CFLAR(8837); FZD7(8324); CD28(940); RPE(6120); COL4A3 |
| | | (1285); HRB(3267); CGREF1(10669); CARD12(58484); RTN4(57142); |
| | | IL1A(3552); BIN1(274); WDR33(55339); ZFHX1B(9839); TLK1(9874); |
| | | NCKAP1(10787); PAX3(5077); CUL3(8452); HDAC4(9759); EDEM1(9695); |
| | | VHL(7428); NR2C2(7182); ZNF660(285349); TDGF1(6997); RBM5(10181); |
| | | APPL(26060); C3orf14(57415); NEK11(79858); RNF7(9616); SMC4L1 |
| | | (10051); PPM1L(151742); PSMD2(5708); TP73L(8626); CIDEC(63924); |
| | | RAF1(5894); DNASE1L3(1776); FHIT(2272); CD47(961); STAG1(10274); |
| | | PIK3CB(5291); TFDP2(7029); SIAH2(6478); WHSC1(7468); HD(3064); CD38 |
| | | (952); HCAP-G(64151); ANAPC4(29945); PDGFRA(5156); LETM1(3954); |
| | | APBB2(323); ATP8A1(10396); UNC5C(8633); DKK2(27123); NEK1 |
| | | (4750); SKP2(6502); NIPBL(25836); CARD6(84674); RAD17(5884); TNPO1 |
| | | (3842); APC(324); DCP2(167227); CSNK1G3(1456); RAD50(10111); SLC26A2 |
| | | (1836); UNC5A(90249); PRLR(5618); BIRC1(4671); ATG12(9140); SEMA6A |
| | | (57556); IRF1(3659); PPP2CA(5515); CDC23(8697); HDAC3(8841); |
| | | CSNK1A1(1452); FBXW11(23291); DUSP1(1843); RIPK1(8737); E2F3 |
| | | (1871); C6orf69(222658); CDKN1A(1026); BAG2(9532); PTP4A1(7803); TTK |
| | | (7272); FOXO3A(2309); TNFAIP3(7128); KIAA1244(57221); HECA(51696); |
| | | SASH1(23328); ATXN1(6310); CUL7(9820); TNFRSF21(27242); MCM3 |
| | | (4172); DST(667); SGK(6446); BCLAF1(9774); PDCD2(5134); AHR(19 |

Table 4-30

| | | |
|---|---|---|
| | | 6); HDAC9(9734); ITGB8(3696); SEPT7(989); EGFR(1956); VKORC1L1(154807); |
| | | FZD1(8321); RINT1(60561); BCAP29(55973); MDFIC(29969); RBM33 |
| | | (155435); CYCS(54205); YWHAG(7532); ZC3HC1(51530); POLR3D |
| | | (661); AGPAT6(137964); UBE2V2(7336); TERF1(7013); C8orf1(734); BIN3 |
| | | (55909); TNFRSF10B(8795); TNFRSF10D(8793); TRIM35(23087); PLAG1 |
| | | (5324); MMP16(4325); CCNE2(9134); TP53INP1(94241); STK3(6788); EXT1 |
| | | (2131); MTSS1(9788); ANKRD15(23189); JAK2(3717); UNC13B(10497); |
| | | DAPK1(1612); ABL1(25); RANBP6(26953); TAF1L(138474); BAG1(573); |
| | | GAS1(2619); PTCH(5727); CTNNAL1(8727); FBXW2(26190); TSC1(7248); |
| | | ZMYND11(10771); TCF8(6935); SIRT1(23411); PTEN(5728); FRAT1 |
| | | (10023); ENTPD7(57089); NEURL(9148); LRRC27(80313); PARD3(56288); |
| | | CUL2(8453); FZD8(8325); ZNF248(57209); ZWINT(11130); COX15(1355); |
| | | TIAL1(7073); MKI67(4288); STIM1(6786); HIPK3(10114); EXT2(2132); |
| | | INCENP(3619); ATM(472); STS-1(84959); EI24(9538); CHEK1(1111); |
| | | IGF2(3481); DKK3(27122); PAK1(5058); SESN3(143686); PPP2R1B(5519); |
| | | CCND2(894); DIP2B(57609); ESPL1(9700); CDK2(1017); GAS2L3(283431); |
| | | BRI3BP(140707); RAN(5901); LMO3(55885); KRAS(3845); SLC38A1 |
| | | (81539); FKBP11(51303); CBX5(23468); DUSP6(1848); PCTK2(5128); ANAPC7 |
| | | (51434); DIABLO(56616); FGF9(2254); ATP8A2(51761); CDK8(1024); |
| | | AKAP11(11215); RFP2(10206); SUGT1(10910); HS6ST3(266722); LATS2 |
| | | (26524); NEK3(4752); KLF12(11278); CDKN3(1033); CGRRF1(10668); |
| | | PSEN1(5663); VASH1(22846); ACIN1(22985); STRN3(29966); EXOC5 |
| | | (10640); CHES1(1112); PPP1R13B(23368); BUB1B(701); MAPK6(5597); |
| | | PAQR5(54852); IGF1R(3480); UBE3A(7337); AVEN(57099); BCL2L10(10017); |
| | | CSNK1G1(53944); CCNF(899); PRKCB1(5579); PAPD5(64282); CYLD |
| | | (1540); CHD9(80205); RBL2(5934); E2F4(1874); CTCF(10664); AXIN1 |
| | | (8312); SIAH1(6477); FTS(64400); CMTM4(146223); PAFAH1B1(5048); GSG2 |
| | | (83903); ALOX15B(247); NF1(4763); CROP(51747); NME1(4830); PPM1E |
| | | (22843); PRKCA(5578); KPNA2(3838); BIRC5(332); PITPNA(5306); |
| | | TP53(7157); BECN1(8678); GJA7(10052); NPTX1(4884); CSNK1D(1453); |
| | | C18orf1(753); MIB1(57534); MAPRE2(10982); MALT1(10892); PHLPP(23239); |
| | | ROCK1(6093); ESCO1(114799); ZNF24(7572); UHRF1(29128); CSPG3 |
| | | (1463); AXL(558); FOSB(2354); SPHK2(56848); ATF5(22809); LENG8 |
| | | (114823); TNFSF14(8740); ZNF443(10224); CARD8(22900); PRND(23627); |
| | | MCM8(84515); PLCB1(23236); PARD6B(84612); BIRC7(79444); CSNK2A1 |
| | | (1457); RBL1(5933); UBE2V1(7335); CTCFL(140690); DIDO1(11083); |
| | | IFNAR1(3454); SON(6651); SLC5A3(6526); MX1(4599); SNF1LK(150094); |
| | | BCL2L13(23786); NF2(4771); MCM5(4174); TNRC6B(23112); PPARA |
| | | (5465); MAPK1(5594); PPM1F(9647); MN1(4330); SMC1L2(27127); CCNB3 |
| | | (85417); TAF1(6872); PGK1(5230); BIRC4(331); STAG2(10735); RP6- |
| | | 213H19.1(51765); DDX26B(203522); AFF2(2334); SMC1L1(8243); CUL4B |
| | | (8450); RP6-166C19.1(255313); MECP2(4204); |
| hsa-miR-202 | 56; 2236; | LCK(3932); RBBP4(5928); UTP11L(51118); MACF1(23499); SH3GLB1(51100); |
| | | LRRC8C(84230); PROK1(84432); BCL9(607); C1orf21(81563); TRAF5 |
| | | (7188); FRAP1(2475); TESK2(10420); PYGO2(90780); FMOD(2331); IL10 |
| | | (3586); MYCN(4613); HK2(3099); BRRN1(23397); RIF1(55183); E2F6 |
| | | (1876); RTKN(6242); IL1A(3552); TLK1(9874); NCKAP1(10787); STK17B |
| | | (9262); CUL3(8452); FANCD2(2177); CTNNB1(1499); TDGF1(6997); PCNP |
| | | (57092); SKIL(6498); KLHL24(54800); SENP2(59343); GSK3B(2932); HD |
| | | (3064); CPEB2(132864); FGF5(2250); PLK4(10733); SPCS3(60559); SLC25A4 |
| | | (291); GABRA4(2557); RCHY1(25898); GHR(2690); NLN(57486); ERBB2IP |
| | | (55914); APC(324); CSNK1G3(1456); PPARGC1B(133522); SLC26A2 |
| | | (1836); CPEB4(80315); MTMR12(54545); FLJ90709(153129); P18SRP |
| | | (285672); SLCO4C1(353189); ATG12(9140); DUSP1(1843); PARC(23113); |
| | | SMAP1(60682); MTO1(25821); SLC16A10(117247); HECA(51696); SASH1 |
| | | (23328); NUDT3(11165); GTPBP2(54676); DST(667); SESN1(27244); |
| | | KATNA1(11104); HDAC9(9734); STAG3(10734); HBP1(26959); POLR3D |
| | | (661); LEPROTL1(23484); UBE2V2(7336); SULF1(23213); RIPK2(8767); |
| | | TNFRSF10B(8795); TNFRSF10A(8797); RECK(8434); C9orf28(89853); TAF1L |
| | | (138474); PTCH(5727); STRBP(55342); TSC1(7248); ZMYND11(10771); |
| | | C10orf9(219771); SIRT1(23411); OIT3(170392); ENTPD7(57089); SFXN2 |
| | | (118980); DOCK1(1793); CPEB3(22849); SMNDC1(10285); DCLRE1A |
| | | (9937); TIAL1(7073); HIPK3(10114); EXT2(2132); TSPAN18(90139); FLJ13848 |
| | | (79829); CCND1(595); ATM(472); CHEK1(1111); WT1(7490); AGBL2 |
| | | (79841); SHANK2(22941); PAK1(5058); CCND2(894); LTBR(4055); E |

Table 4-31

| | | |
|---|---|---|
| | | SPL1(9700); GDF11(10220); HMGA2(8091); MDM2(4193); TRHDE(29953); |
| | | APAF1(317); LRP6(4040); BCAT1(586); LALBA(3906); DUSP6(1848); |
| | | CHFR(55743); FGF9(2254); BRCA2(675); RB1(5925); LATS2(26524); STARD13 |
| | | (90627); FOXO1A(2308); KIAA0329(9895); HNRPC(3183); GZMB(3002); |
| | | EGLN3(112399); CDKL1(8814); FLJ33387(161145); LTBP2(4053); |
| | | TGFB3(7043); CHES1(1112); DICER1(23405); ARID3B(10620); CSK(1445); |
| | | LOC440295(440295); IGF1R(3480); NIPA1(123606); BMF(90427); CSNK1G1 |
| | | (53944); RBL2(5934); ADCY9(115); GSPT1(2935); SMPD3(55512); |
| | | KIAA1609(57707); FZD2(2535); CROP(51747); YPEL2(388403); MNT(4335); |
| | | METT10D(79066); SPAG5(10615); GAS2L2(246176); TBC1D16(125058); |
| | | CSNK1D(1453); CCDC5(115106); UHRF1(29128); ZNF443(10224); |
| | | TGFB1(7040); PLCB1(23236); STK4(6789); PARD6B(84612); VAPB(9217); |
| | | RBL1(5933); TNRC6B(23112); EP300(2033); PPARA(5465); OGT(8473); |
| | | PGK1(5230); CAPN6(827); PDCD8(9131); |
| hsa-miR-204 | 57; 693; 694; | PRKCZ(5590); PHF13(148479); PRDM2(7799); NBL1(4681); CDC42(998); |
| hsa-miR-211 | 695; 696; | NUDC(10726); KHDRBS1(10657); HDAC1(3065); MACF1(23499); CDKN2C |
| | 697; 698; 699; | N2C(1031); CDC7(8317); CDC14A(8556); RAP1A(5906); BCL9(607); DAP3 |
| | 700; 701; | (7818); POGK(57645); RGS2(5997); CDC2L2(985); CTNNBIP1(56998); |
| | 702; 703; | PRAMEF8(391002); CASP9(842); RCC2(55920); YARS(8565); SASS6(163786); |
| | 704; 705; 706; | NOTCH2(4853); MCL1(4170); GPATC4(54865); ASPM(259266); WDR26 |
| | 707; 708; | (80232); TMEM63A(9725); ZNF496(84838); SOX11(6664); MAPRE3 |
| | 58; 709; 710; | (22924); BIRC6(57448); MSH2(4436); TCF7L1(83439); PROC(5624); TAIP-2 |
| | 711; 712; | (80034); ZAK(51776); CFLAR(8837); CASP8(841); CD28(940); ALS2C27R19 |
| | 713; 714; | (117583); COL4A3(1285); CGREF1(10669); EIF2AK2(5610); TMEM127 |
| | 2237; | (55654); ANAPC1(64682); IL1B(3553); BIN1(274); ERCC3(2071); TLK1 |
| | | (9874); NCKAP1(10787); CUL3(8452); HDAC4(9759); VHL(7428); RBMS3 |
| | | (27303); PDCD6IP(10015); NKTR(4820); RBM5(10181); RBM15B(29890); |
| | | GNL3(26354); TP73L(8626); OPA1(4976); NEK4(6787); PIK3CB(5291); |
| | | SMARCA3(6596); HD(3064); FGF5(2250); AFF1(4299); BTC(685); PRKG2 |
| | | (5593); DKK7(27123); ELOVL6(79071); SFRP2(6423); PDCD6(10016); |
| | | SKP2(6502); NIPBL(25836); CARD6(84674); ERBB2IP(55914); F2R(2149); |
| | | APC(324); ARHGAP26(23092); SGCD(6444); SEMA5A(9037); PRLR(5618); |
| | | MCC(4163); ATG12(9140); SEMA6A(57556); CDC23(8697); FGF1(2246); |
| | | FBXW11(23291); RIPK1(8737); E2F3(1871); AIF1(199); VEGF(7422); |
| | | RUNX2(860); PTP4A1(7803); TTK(7272); TNFAIP3(7128); RP5-875H10.1 |
| | | (389432); NEDD9(4739); ATXN1(6310); DHX16(8449); IHPK3(117283); |
| | | TNFRSF21(27242); DST(667); DJ122O8.2(57226); ATG5(9474); BCLAF1 |
| | | (9774); MAP3K5(4217); LATS1(9113); AHR(196); HDAC9(9734); CREB5 |
| | | (9586); SEPT7(989); FZD1(8321); RINT1(60561); BCAP29(55973); TRIAD3 |
| | | (54476); SOSTDC1(25928); DRCTNNB1A(84668); CYCS(54205); HOXA13 |
| | | (3209); ELMO1(9844); SFRP4(6424); INHBA(3624); YWHAG(7532); CDK6 |
| | | (1021); FIS1(51024); SLC37A3(84255); POLR3D(661); PTK2B(2185); |
| | | ESCO2(157570); UNC5D(137970); TACC1(6867); RIPK2(8767); GML(2765); |
| | | BIN3(55909); TNFRSF10D(8793); ADRA1A(148); CLU(1191); DUSP4 |
| | | (1846); SFRP1(6422); TP53INP1(94241); MTSS1(9788); JAK2(3717); NUDT2 |
| | | (318); RECK(8434); MGC21881(389741); NTRK2(4915); DAPK1(1612); |
| | | IFNW1(3467); TAF1L(138474); GAS1(2619); PTCH(5727); TNFSF15 |
| | | (9966); TNFSF8(944); TRAF1(7185); TSC1(7248); C10orf9(219771); SIRT1 |
| | | (23411); SGPL1(8879); UNC5B(219699); PTEN(5728); KIF11(3832); PDCD11 |
| | | (22984); CSPG6(9126); TCF7L2(6934); DOCK1(1793); IL2RA(3559); |
| | | PARD3(56288); CUL2(8453); DLG5(9231); FBXW4(6468); SH3PXD2A |
| | | (9644); TIAL1(7073); MXI67(4288); STIM1(6786); WEE1(7465); MDK(4192); |
| | | LGALS12(85329); FLJ13848(79829); LOC220070(220070); BIRC3(330); |
| | | BIRC2(329); LOH11CR2A(4013); CHEK1(1111); EIF4G2(1982); HRASLS3 |
| | | (11145); MAP3K11(4296); BRMS1(25855); FGF3(2248); FZD4(8322); |
| | | SESN3(143686); BCL9L(283149); CCND2(894); LTBR(4055); MDM2(4193); |
| | | APAF1(317); ANKRD13(88455); BCAT1(586); KRAS(3845); C12orf22 |
| | | (81566); DUSP6(1848); GALNT4(8693); NUAK1(9891); PPP1CC(5501); |
| | | MPHOSPH9(10198); HSMPP8(54737); BRCA2(675); EXOSC8(11340); RFP2 |
| | | (10206); ING1(3621); TFDP1(7027); LATS2(26524); STARD13(90627); |
| | | FOXO1A(2308); KLF12(11278); LIG4(3981); BCL2L2(599); PNN(5411); |
| | | MNAT1(4331); PSEN1(5663); VASH1(22846); GPR65(8477); ACIN1(22985); |
| | | CIDEB(27141); GZMH(2999); STRN3(29966); DLG7(9787); SYNJ2BP |
| | | (55333); LTBP2(4053); TGFB3(7043); CHES1(1112); BTBD7(55727); GREM1 |
| | | (26585); SPRED1(161742); THBS1(7057); BUB1B(701); FGF7(2252) |

Table 4-32

| | | |
|---|---|---|
| | | ; MAPK6(5597); SLC12A6(9990); BMF(90427); BNIP2(663); TSC2(7249); |
| | | TRAF7(84231); CCNF(899); TNFRSF12A(51330); DNAJA3(9093); RBL2 |
| | | (5934); NOL3(8996); ELMO3(79767); GSPT1(2935); SEPT1(1731); FTS(64400); |
| | | TXNL4B(54957); ALOX12(239); CCDC55(84081); NF1(4763); LIG3 |
| | | (3980); CDC6(990); DHX8(1659); MAPT(4137); CROP(51747); NME2(4831); |
| | | PPM1E(22843); PRKCA(5578); MNT(4335); P2RX1(5023); NALP1(22861); |
| | | GAS7(8522); SPAG5(10615); GAS2L2(246176); SEPT4(5414); PTRH2 |
| | | (51651); DSG3(1830); MAPRE2(10982); DCC(1630); ROCK1(6093); ESCO1 |
| | | (114799); BCL2(596); CHAF1A(10036); DNM2(1785); CASP14(23581); APOE |
| | | (348); FOSB(2354); SPHK2(56848); PPP2R1A(5518); PRKACA(5566); |
| | | DPF1(8193); DEDD2(162989); CARD8(22900); KLK10(5655); TRIB3(57761); |
| | | ATRN(8455); MCM8(84515); PLCB1(23236); MAPRE1(22919); CEP250 |
| | | (11190); UBE2C(11065); CD40(958); RAB22A(57403); BCL2L1(598); |
| | | RBL1(5933); PTPRT(11122); CTCFL(140690); SYCP2(10388); DIDO1(11083); |
| | | USP16(10600); CHAF1B(8208); DYRK1A(1859); MX1(4599); BRWD1 |
| | | (54014); ADORA2A(135); NF2(4771); TNRC6B(23112); GTSE1(51512); |
| | | PPM1F(9647); MN1(4330); RBM9(23543); CARD10(29775); DMC1(11144); |
| | | HDAC6(10013); TMEM28(27112); TAF1(6872); SPRY3(10251); SEPT6 |
| | | (23157); CUL4B(8450); PDCD8(9131); ARHGEF6(9459); |
| has-miR-205 | 59; 715; 716 | PRKCZ(5590); SFN(2810); RBBP4(5928); MACF1(23499); CDKN2C(1031); |
| | 717; 718; | BCL9(607); MDM4(4194); IL19(29949); IL24(11009); FRAP1(2475); FAF1 |
| | 719; 720; 721; | (11124); NOTCH2(4853); ASPM(259266); IL10(3586); TP53BP2(7159); |
| | 722; 723; | FH(2271); PRKCE(5581); MSH2(4436); HTRA2(27429); HK2(3099); TCF7L1 |
| | 724; 725; | (83439); RIF1(55183); CFLAR(8337); COL4A3(1285); PSMD1(5707); |
| | 726; 727; 728; | SEPT2(4735); YWHAQ(10971); RTN4(57142); TGFA(7039); FLJ10081(55683); |
| | 729; 730; | EDAR(10913); BUB1(699); ANAPC1(64682); IL1A(3552); BIN1(274); |
| | 2238; | TLK1(9874); DKFZp434O0527(255101); PAX3(5077); FANCD2(2177); |
| | | PCAF(8850); PDCD6IP(10015); CTNNB1(1499); SENP2(59343); RAF1(5894); |
| | | STAG1(10274); TFDP2(7029); ATR(545); HD(3064); HCAP-G(64151); |
| | | SEPT11(55752); CCNG2(901); NFKB1(4790); FGF2(2247); PLK4(10733); |
| | | GAK(2580); FLJ90013(202018); BTC(685); UNC5C(8633); DKK2(27123); |
| | | SFRP2(6423); NEK1(4750); CASP3(836); NIPBL(25836); ERBB2IP(55914); |
| | | F2R(2149); APC(324); TCF7(6932); AYTL2(79888); PRLR(5618); DHFR |
| | | (1719); SEMA6A(57556); PFDN1(5201); FGF1(2246); CSNK1A1(1452); |
| | | LY86(9450); E2F3(1871); LTA(4049); CSNK2B(1460); PARC(23113); VEGF |
| | | (7422); PTP4A1(7803); TNFAIP3(7128); SASH1(23328); QKI(9444); CUL7 |
| | | (9820); DST(667); ATG5(9474); FRK(2444); MAP3K5(4217); PLAGL1(5325); |
| | | AGPAT4(56895); PDCD2(5134); HDAC9(9734); SEPT7(989); EGFR |
| | | (1956); PFTK1(5218); RINT1(60561); CARD4(10392); INHBA(3624); CDK6 |
| | | (1021); DLGAP2(9228); POLR3D(661); PTK2B(2185); TACC1(6867); UBE2V2 |
| | | (7336); SULF1(23213); EBAG9(9166); LZTS1(11178); TNFRSF10B |
| | | (8795); TRIM35(23087); CLU(1191); RAB11FIP1(80223); WHSC1L1(54904); |
| | | TP53INP1(94241); NUDT2(318); UNC13B(10497); DAPK1(1612); C9orf47 |
| | | (286223); INVS(27130); CDK9(1025); TRAF2(7186); IFNA2(3440); CDKN2B |
| | | (1030); TAF1L(138474); TRIM14(9830); TNFSF8(944); TRAF1(7185); |
| | | TSC1(7248); CARD9(64170); ZMYND11(10771); PTEN(5728); FAS |
| | | (355); FRAT1(10023); BTRC(8945); CSPG6(9126); TCF7L2(6934); BAG3(9531); |
| | | PARD3(56288); DLG5(9231); DCLRE1A(9937); MKI67(4288); STIM1 |
| | | (6786); HIPK3(10114); F2(2147); INCENP(3619); CCND1(595); BIRC2(329); |
| | | CUL5(8065); ATM(472); FGF4(2249); PAK1(5058); CASP4(837); ZW10 |
| | | (9183); BCL9L(283149); PTMS(5763); CCT2(10576); APAF1(317); LRP6 |
| | | (4040); FAIM2(23017); DDIT3(1649); CDK4(1019); PCTK2(5128); ATXN2 |
| | | (6311); BRCA2(675); ING1(3621); CUL4A(8451); TFDP1(7027); FOXO1A |
| | | (2308); KLF12(11278); BCL2L2(599); PNN(5411); TRAF3(7187); SIVA(10572); |
| | | STRN3(29966); CDKL1(8814); DLG7(9787); WDR22(8816); NEK9(91754); |
| | | MOAP1(64112); AKT1(207); FGF7(2252); Gcom1(145781); CCNB2 |
| | | (9133); CSK(1445); IGF1R(3480); BNIP2(663); DAPK2(23604); CCNF(899); |
| | | KREMEN2(79412); CARD15(64127); CYLD(1540); ELMO3(79767); NFAT5 |
| | | (10725); MAPK3(5595); SLAH1(6477); CMTM4(146223); PCOLN3(5119); |
| | | HIC1(3090); PAFAH1B1(5048); ALOX12(239); NLK(51701); TRAF4 |
| | | (9618); RAB11FIP4(84440); LIG3(3980); PRKCA(5578); CARD14(79092); |
| | | ANAPC11(51529); GPS2(2874); SPAG5(10615); GAS2L2(246176); PPARBP |
| | | (5469); SEPT4(5414); PTRH2(51651); ERN1(2081); DCC(1630); PHLPP |
| | | (23239); FLJ44881(400661); YES1(7525); ROCK1(6093); BCL2(596); PARD6G |
| | | (84552); HMG20B(10362); UBA52(7311); NDUFA13(51079); CCN |

Table 4-33

| | | |
|---|---|---|
| | | E1(898); AXL(558); APOE(348); CDC37(11140); PPP1R13L(10848); CARD8 |
| | | (22900); ZNF606(80095); PLCB1(23236); MAPRE1(22919); STK4(6789); |
| | | E2F1(1869); UBE2V1(7335); CTCFL(140690); SON(6651); BRWD1(54014); |
| | | SNF1LK(150094); BCL2L13(23786); CDC45L(8318); SEPT5(5413); |
| | | GAS2L1(10634); YWHAH(7533); SEPT3(55964); PPM1F(9647); DMC1(11144); |
| | | TOB2(10766); NHP2L1(4809); MAPK12(6300); CCNB3(85417); MLLT7 |
| | | (4303); TAF1(6872); AGTR2(186); IKBKG(8517); PPP2R3B(28227); |
| | | FIGF(2277); RPS6KA3(6197); SMC1L1(8243); BTK(695); RP13-383K5.1 |
| | | (55285); SEPT6(23157); |
| hsa-miR-208 | 60; 731; 732; | SKI(6497); PHF13(148479); KIF1B(23095); VPS13D(55187); NPAL3(57185); |
| | 733; 734; | ZMYM1(79830); ZMYM4(9202); MTF2(22823); GPR88(54112); SNAPAP |
| | 897; 898; 2239; | (23557); KIAA0446(9673); ATP1A2(477); MIPZL1(9019); SRGAP2(23380); |
| | | KCTD3(51133); FBXO28(23219); SLC35F3(148641); KMO(8564); CDC2L2 |
| | | (985); RPL22(6146); ICMT(23463); DFFA(1676); E2F2(1870); HEYL |
| | | (26508); F3(2152); RIPK5(25778); SLC45A3(85414); ENAH(55740); CHML |
| | | (1122); ZNF496(84838); RSNL2(79745); ABCG8(64241); ANTXR1(84168); |
| | | MAP4K4(9448); ACOXL(55289); CCNT2(905); PMS1(5378); ADAM23 |
| | | (8745); ALPI(248); HS1BP3(64342); RBJ(51277); SP3(6670); KIAA1715(80856); |
| | | TTN(7273); NCKAP1(10787); RAPH1(65059); HDAC4(9759); MTERFD2 |
| | | (130916); SRPRB(58477); TLOC1(7095); B3GNT5(84002); SLC6A20 |
| | | (54716); LTF(4057); MGC40579(256356); KLHL6(89857); LOC285382(285382); |
| | | CPEB2(132864); HCAP-G(64151); EREG(2069); CLCN3(1182); RPL9 |
| | | (6133); ASAHL(27163); CXCL9(4283); CAMK2D(817); SLC7A11(23657); |
| | | NPY1R(4886); APC(324); SLC22A5(6584); UBE2D2(7322); PCDHB3 |
| | | (56132); NPM1(4869); MYO10(4651); UGT3A1(133688); DKFZP781I1119 |
| | | (166968); ADAMTS6(11174); FOXQ1(94234); RREB1(6239); TUBB(203068); |
| | | RP5-875H10.1(389432); QKI(9444); RP3-398D13.1(285780); HIST1H2BC |
| | | (8347); C6orf125(84300); UBE2J1(51465); HS3ST5(222537); STX7 |
| | | (8417); LATS1(9113); FOXK1(221937); WBSCR17(64409); LMTK2(22853); |
| | | DLD(1738); TES(26136); LEP(3952); KIAA0265(23008); LRRC61(65999); |
| | | WBSCR16(81554); C7orf21(83590); EFHA2(286097); SLC7A2(6542); HMBOX1 |
| | | (79618); PHF20L1(51105); BIN3(55909); PXMP3(5828); MGC21881 |
| | | (389741); C9orf47(286223); STX17(55014); INVS(27130); STXBP1(6812); |
| | | C9orf7(11094); AK3(50808); SMU1(55234); KIAA0367(23273); HEMGN |
| | | (55363); AKNA(80709); FAM102A(399665); ECHDC3(79746); PCGF4 |
| | | (648); ANXA8(244); SIRT1(23411); HIF1AN(55662); CNNM2(54805); KIAA1600 |
| | | (57700); GPR26(2849); PTPRE(5791); DIP2C(22982); CUL2(8453); |
| | | ZNF25(219749); C10orf13(143282); MGEA5(10724); SH3PXD2A(9644); |
| | | PDZD8(118987); ZNF215(7762); USP47(55031); TCP11L1(55346); CUL5 |
| | | (8065); JAM3(83700); VPS26B(112936); FLJ20294(55626); SSRP1(6749); |
| | | CDCA5(113130); SYVN1(84447); FLJ38159(220388); ZW10(9183); IGSF4 |
| | | (23705); BCL9L(283149); ETS1(2113); TRHDE(29953); APAF1(317); RIC8B |
| | | (55188); ACACB(32); RECQL(5965); VDR(7421); SMARCC2(6601); E2F7 |
| | | (144455); NUAK1(9891); RIMBP2(23504); AKAP11(11215); ATP11A |
| | | (23250); KLF12(11278); DOCK9(23348); DHRS4(10901); DHRS4L2(317749); |
| | | PPP2R5C(5527); NFKBIA(4792); MAX(4149); DIO2(1734); KIAA1446 |
| | | (57596); SGNE1(6447); RYR3(6263); TPM1(7168); PSMA4(5685); IL16(3603); |
| | | LOC440295(440295); SCAND2(54581); NIPA1(123606); TLE3(7090); |
| | | STARD5(80765); PRO0149(29035); MGC35048(124152); ATP2A1(487); |
| | | DHODH(1723); JPH3(57338); C16orf28(65259); NSMCE1(197370); BOLA2 |
| | | (552900); N4BP1(9683); CSNK2A2(1459); ST3GAL2(6483); SLC7A5(8140); |
| | | ZFP3(124961); FLJ35934(400579); NLK(51701); MGC19764(162394); |
| | | TUBG2(27175); FLJ42842(440446); HLF(3131); PRKCA(5578); TNRC6C |
| | | (57690); FN3KRP(79672); MYO1C(4641); ANKFY1(51479); GAS7(8522); |
| | | SREBF1(6720); THRAP1(9969); CYB561(1534); DDX5(1655); SLC16A6 |
| | | (9120); SUMO2(6613); GNAL(2774); LIPG(9388); SMAD4(4089); SOCS6 |
| | | (9306); NPC1(4864); PSTPIP2(9050); BRUNOL5(60680); FZR1(51343); ILF3 |
| | | (3609); FLJ21742(84167); UPF1(5976); ZBTB32(27033); SPIB(6689); |
| | | CACNG7(59284); ZNF135(7694); SLC25A23(79085); ELL(8178); OPA3(80207); |
| | | FLJ10781(55228); KLK10(5655); VISA(57506); SLC35C2(51006); |
| | | UBE2V1(7335); KCNG1(3755); YTHDF1(54915); BTBD4(140685); SON |
| | | (6651); UBASH3A(53347); DIP2A(23181); SYNJ1(8867); C21orf55(54943); |
| | | RANBP1(5902); FLJ42953(400892); BCR(613); FLJ31568(150244); RP5- |
| | | 1119A7.4(80020); DDX17(10521); CELSR1(9620); CXorf15(55787); CNKSR2 |
| | | (22866); ZC3H12B(340554); PCDH11X(27328); ARSD(414); TAB3(25 |

Table 4-34

| | | |
|---|---|---|
| | | 7397); ARHGEF9(23229); PCDH11Y(83259); |
| hsa-miR-222 | 61; 735; 736; | CAMTA1(23261); PRDM2(7799); NBPF3(84224); NSUN4(387338); CDC7 |
| | 737; 738; | (8317); SYCP1(6847); CKS1B(1163); UHMK1(127933); MPZL1(9019); DNM3 |
| | 739; 740; 741; | (26052); NAV1(89796); MDM4(4194); FBXO28(23219); CCNL2(81669); |
| | 742; 743; | RCC2(55920); RIMS3(9783); BCL10(8915); SASS6(163786); SORT1(6272); |
| | 899; 900; | TNFSF18(8995); ZBTB41(360023); TP53BP2(7159); ENAH(55740); |
| | 901; 902; 903; | SPTBN1(6711); BRRN1(23397); MGC5509(79074); ACTR3(10096); RIF1 |
| | 904; 905; | (55183); ZAK(51776); CASP10(843); HRB(3267); KIAA1212(55704); FLJ16008 |
| | 906; 907; | (339761); ARL5A(26225); STAT1(6772); CLK1(1195); CUL3(8452); FANCD2 |
| | 908; 909; 910; | (2177); CAPN7(23473); PDCD6IP(10015); STAC(6769); ITGA9(3680); |
| | 911; 912; | KLHL18(23276); RBM5(10181); IL17RB(55540); APPL(26060); TFG |
| | 2240; | (10342); AADAC(13); SMC4L1(10051); CLDN11(5010); OPA1(4976); VGLL4 |
| | | (9686); SLC4A7(9497); NME6(10201); PRKAR2A(5576); AER61(285203); |
| | | SNX4(8723); HD(3064); HCAP-G(64151); FGF5(2250); C4orf12(404201); |
| | | FGF2(2247); APBB2(323); UGT2B15(7366); PRKG2(5593); NAP1L5 |
| | | (266812); NIPBL(25836); MARVELD2(153562); APC(324); MFAP3(4238); |
| | | GABRA1(2554); PRLR(5618); C6(729); PLCXD3(345557); ATG12(9140); |
| | | CDC25C(995); PANK3(79646); C6orf151(154007); RBM24(221662); ZNF192 |
| | | (7745); TNF(7124); HLA-DQA1(3117); SASH1(23328); RP11-145H9.1 |
| | | (340156); ATXN1(6310); TRAM2(9697); DST(667); CCNC(892); OSTM1(28962); |
| | | CDC2L6(23097); HBS1L(10767); LATS1(9113); SEPT7(989); EGFR |
| | | (1956); ADAM22(53616); ZNF655(79027); IFRD1(3475); TRIAD3(54476); |
| | | PMS2(5395); OSBPL3(26031); SCAP2(8935); GRB10(2887); YWHAG |
| | | (7532); CDK6(1021); HIPK2(28996); KBTBD11(9920); BNIP3L(665); PTK2B |
| | | (2185); SULF1(23213); LRRCC1(85444); ZNF704(619279); RECK(8434); |
| | | DAPK1(1612); TRAF2(7186); CDKN2B(1030); TRIM14(9830); TRAF1 |
| | | (7185); PPP6C(5537); TSC1(7248); RAB18(22931); CDC2(983); SIRT1(23411); |
| | | UNC5B(219699); SAMD8(142891); FAM35A(54537); HECTD2(143279); |
| | | ENTPD7(57089); DNMT2(1787); PARD3(56288); CUL2(8453); CXCL12 |
| | | (6387); MARCH8(220972); CPEB3(22849); FRAT2(23401); DCLRE1A |
| | | (9937); HIPK3(10114); ZFP91(80829); MLL(4297); CDKN1C(1028); PAK1 |
| | | (5058); SESN3(143686); PDGFD(80310); BCL9L(283149); CCND2(894); NTF3 |
| | | (4908); LTBR(4055); BCL2L14(79370); CDKN1B(1027); PA2G4(5036); |
| | | MDM2(4193); PLXNC1(10154); APAF1(317); CUTL2(23316); FLJ46363 |
| | | (400002); LRP6(4040); MPHOSPH9(10198); FGF9(2254); BRCA2(675); ING1 |
| | | (3621); C13orf10(64062); SOCS4(122809); KTN1(3895); RGS6(9628); |
| | | VASH1(22846); TRAF3(7187); ACIN1(22985); TGFB3(7043); BAG5(9529); |
| | | THBS1(7057); TCF12(6938); NIPA1(123606); BMF(90427); RFXDC2(64864); |
| | | CSNK1G1(53944); CP110(9738); SBK1(388228); CYLD(1540); RBL2 |
| | | (5934); CTCF(10664); MAPK3(5595); MIS12(79003); TNFSF12(8742); |
| | | NLK(51701); TADA2L(6871); PPM1D(8493); KPNA2(3838); MAP2K6(5608); |
| | | NXN(64359); AXIN2(8313); SOCS3(9021); MAPRE2(10982); SERPINB2 |
| | | (5055); BCL2(596); FVT1(2531); ZADH2(284273); FZR1(51343); ZNF181 |
| | | (339318); MAP3K10(4294); BBC3(27113); VAPB(9217); CSNK2A1(1457); |
| | | SYCP2(10388); DYRK1A(1859); BTG3(10950); CRKL(1399); MAPK1 |
| | | (5594); DMC1(11144); LOC401620(401620); DKC1(1736); SMC1L1(8243); |
| | | AMMECR1(9949); CLIC2(1193); |
| hsa-miR-299-3p | 62; 2241; | PRKCZ(5590); TNFRSF1B(7133); CDC42(998); SESN2(83667); LCK(3932); |
| | | RBBP4(5928); MACF1(23499); PLK3(1263); CDC7(8317); PROK1(84432); |
| | | ADORA1(134); TRAF5(7188); TGFB2(7042); C1orf95(375057); TNFRSF14 |
| | | (8764); TNFRSF9(3604); CTNNBIP1(56998); DFFA(1676); E2F2(1870); |
| | | FAF1(11124); MAPRE3(22924); KCNIP3(30818); TAIP-2(80034); ZAK |
| | | (51776); CD28(940); COL4A3(1285); SCLY(51540); SEPT2(4735); E2F6 |
| | | (1876); EIF2AK2(5610); BUB1(699); ANAPC1(64682); IL1B(3553); PAX3 |
| | | (5077); PCAF(8850); NEK11(79858); TP73L(8626); OPA1(4976); RAF1(5894); |
| | | CD47(961); STAG1(10274); HD(3064); EREG(2069); DKFZp761B107 |
| | | (91050); DKK2(27123); APC(324); CSNK1G3(1456); RAD50(10111); TCF7 |
| | | (6932); SGCD(6444); DAP(1611); PRLR(5618); MCC(4163); SEMA6A(57556); |
| | | FSTL4(23105); CSNK1A1(1452); RNF130(55819); FLJ45422(441140); |
| | | CSNK2B(1460); FOXP4(116113); PARC(23113); VEGF(7422); PTP4A1 |
| | | (7803); SLC35F1(222553); MDC1(9656); SUPT3H(8464); DST(667); LATS1 |
| | | (9113); EGFR(1956); STAG3(10734); CASP2(835); CUL1(8454); TRIAD3 |
| | | (54476); PMS2(5395); CYCS(54205); CARD4(10392); ELMO1(9844); ZNF705CP |
| | | (389631); TNFRSF10C(8794); UNC5D(137970); LZTS1(11178); BIN3 |
| | | (55909); TNFRSF10B(8795); ADRA1A(148); EXT1(2131); NTRK2(491 |

Table 4-35

| | | |
|---|---|---|
| | | 5); PAPPA(5069); TAF1L(138474); SEMA4D(10507); PTCH(5727); TRAF1 |
| | | (7185); ZMYND11(10771); UNC5B(219699); LZTS2(84445); PDCD11(22984); |
| | | TCF7L2(6934); DOCK1(1793); IL2RA(3559); KCNMA1(3778); DCLRE1A |
| | | (9937); HIPK3(10114); TSPAN18(90139); F2(2147); INCENP(3619); |
| | | LRP5(4041); CHEK1(1111); IGF2(3481); CDKN1C(1028); HRASLS3(11145); |
| | | MAP3K11(4296); FZD4(8322); CASP1(834); BTG4(54766); PPP2R1B |
| | | (5519); ZW10(9183); PCSK7(9159); BCL9L(283149); TNFRSF7(939); PTPN6 |
| | | (5777); PDE1B(5153); SLC16A7(9194); TCF1(6927); KNTC1(9735); HDAC7A |
| | | (51564); FAIM2(23017); DDIT3(1649); PAWR(5074); MPHOSPH9 |
| | | (10198); ING1(3621); ATP11A(23250); CSNK1A1L(122011); FOXO1A(2308); |
| | | TRAF3(7187); JPH4(84502); STRN3(29966); LTBP2(4053); CCNB2(9133); |
| | | IGF1R(3480); CSNK1G1(53944); TSC2(7249); TRAF7(84231); CCNF |
| | | (899); RBL2(5934); CDT1(81620); CDK10(8558); SEPT1(1731); DNAJA2 |
| | | (10294); SLC12A4(6560); TNFSF12(8742); NLK(51701); NF1(4763); NGFR |
| | | (4804); CROP(51747); PRKCA(5578); SLC9A3R1(9368); CARD14(79092); |
| | | MNT(4335); P2RX1(5023); NALP1(22861); GAS7(8522); GAS2L2(246176); |
| | | SEPT4(5414); SOCS3(9021); PARD6G(84552); FZR1(51343); BCL3 |
| | | (602); APOE(348); FOSB(2354); ATF5(22809); TNFSF14(8740); ZNF443(10224); |
| | | ERF(2077); MCM8(84515); PLCB1(23236); MAPRE1(22919); STK4 |
| | | (6789); ELMO2(63916); DIDO1(11083); KREMEN1(83999); EP300(2033); |
| | | GTSE1(51512); MAPK8IP2(23542); MGC16703(113691); MAPK1(5594); |
| | | KIAA1904(114794); DMC1(11144); TAF1(6872); SMC1L1(8243); SEPT6 |
| | | (23157); HCFC1(3054); |
| hsa-miR-299-5p | 63; 2242; 2243; | KIF1B(23095); RBBP4(5928); MACF1(23499); LAX1(54900); TGFB2(7042); |
| | | CIAS1(114548); CLSPN(63967); GFI1(2672); RSBN1(54665); TSPAN2 |
| | | (10100); S100A6(6277); GLRX2(51022); FH(2271); RIF1(55183); ZAK(51776); |
| | | CD28(940); CAB39(51719); PSMD1(5707); DQX1(165545); CLK1(1195); |
| | | BARD1(580); CUL3(8452); HDAC4(9759); TDGF1(6997); SMC4L1(10051); |
| | | SENP2(59343); OPA1(4976); RAF1(5894); SEC22L3(9117); ROBO1 |
| | | (6091); TFDP2(7029); ATR(545); SIAH2(6478); CPEB2(132864); RBPSUH |
| | | (3516); NFKB1(4790); PLK4(10733); RASSF6(166824); LEF1(51176); GLRA3 |
| | | (8001); LOC285636(285636); MRPS30(10884); ERBB2IP(55914); APC |
| | | (324); PRKAA1(5562); ATG12(9140); SEMA6A(57556); CD14(929); FGF1 |
| | | (2246); DCTN4(51164); FBXW11(23291); PRPF4B(8899); CDKN1A(1026); |
| | | PTP4A1(7803); TRAM2(9697); DST(667); ATG5(9474); SGK(6446); PLAGL1 |
| | | (5325); HDAC9(9734); Ells1(222166); TRIAD3(54476); CARD4(10392); |
| | | PURB(5814); GRB10(2887); FAM3C(10447); SLC35B4(84912); TRIM35 |
| | | (23087); RAD21(5885); RECK(8434); IFNW1(3467); IFNA2(3440); TAF1L |
| | | (138474); PTCH(5727); CTNNAL1(8727); DKK1(22943); PHYHIPL(84457); |
| | | SH2D4B(387694); FAS(355); BTRC(8945); INPP5F(22876); INPP5A |
| | | (3632); ITIH5(80760); PARD3(56288); TIAL1(7073); HIPK3(10114); PDHX |
| | | (8050); PRSS23(11098); EIF4G2(1982); HRASLS3(11145); HBXAP(51773); |
| | | SURB7(9412); ESPL1(9700); LRP6(4040); KITLG(4254); MPHOSPH9 |
| | | (10198); HSMPP8(54737); CDC16(8881); KCTD12(115207); DCT(1638); |
| | | C14orf24(283635); TRAF3(7187); DLG7(9787); CHES1(1112); PLDN(26258); |
| | | ABHD2(11057); C15orf29(79768); GNPTG(84572); MKL2(57496); CTCF |
| | | (10664); CDK10(8558); SIAH1(6477); PAFAH1B1(5048); NF1(4763); GAS7 |
| | | (8522); POLDIP2(26073); HDAC5(10014); MALT1(10892); FZR1(51343); |
| | | LGALS7(3963); NALP2(55655); FLJ26175(388566); ZNF585A(199704); |
| | | SIRT2(22933); PLCB1(23236); CSE1L(1434); PCNA(5111); ELMO2(63916); |
| | | DIDO1(11083); GAS2L1(10634); TNRC6B(23112); CLCN5(1184); NLGN3 |
| | | (54413); TAF1(6872); CLDN2(9075); |
| hsa-miR-302a* | 64; | SAMD11(148398); TP73(7161); HDAC1(3065); SH3GLB1(51100); RAP1A |
| | | (5906); FRAP1(2475); RCC2(55920); SLC9A2(6549); NAB1(4664); BIN1(274); |
| | | CUL3(8452); HDAC4(9759); KNG1(3827); OPA1(4976); MINA(84864); |
| | | GSK3B(2932); ZNF148(7707); MRPL3(11222); STAG1(10274); TBC1D14 |
| | | (57533); PAICS(10606); FGF5(2250); APBB2(323); SFRP2(6423); RNF130 |
| | | (55819); TCF21(6943); MYCT1(80177); TRAM2(9697); SESN1(27244); |
| | | AHR(196); STK17A(9263); RINT1(60561); CALU(813); JAZF1(221895); ELMO1 |
| | | (9844); INHBA(3624); CDK6(1021); DKK4(27121); ZNF406(57623); |
| | | C9orf47(286223); FANCG(2189); GNAQ(2776); TSC1(7248); ZMYND11 |
| | | (10771); PTEN(5728); TMEM20(159371); SUFU(51684); PDCD11(22984); |
| | | CUL2(8453); MKI67(4288); HIPK3(10114); BIRC3(330); CUL5(8065); LOH11CR2A |
| | | (4013); APBB1(322); CNAP1(9918); KNTC1(9735); MPHOSPH9 |
| | | (10198); HSMPP8(54737); ATP8A2(51761); PAN3(255967); SUGT1(109 |

Table 4-36

| | | |
|---|---|---|
| | | 10); FLJ40296(122183); CLN5(1203); TRAF3(7187); KNS2(3831); BAG5 |
| | | (9529); KLF13(51621); SMAD3(4088); GABRB3(2562); BMF(90427); BNIP2 |
| | | (663); PDPR(55066); FTS(64400); TRAF4(9618); MAPT(4137); HLF(3131); |
| | | PRKCA(5578); NALP1(22861); BECN1(8678); CHAF1A(10036); CASP14 |
| | | (23581); PPP2R1A(5518); PCSK2(5126); MAPRE1(22919); STK4(6789); |
| | | CSNK2A1(1457); TTC3(7267); APP(351); SNF1LK(150094); CDC45L(8318); |
| | | EP300(2033); CHEK2(11200); SHOX(6473); TAF1(6872); F8A1(8263); |
| | | HCFC1(3054); |
| hsa-miR-302b* | 65; 744; 745; | KHDRBS1(10657); MACF1(23499); CDC20(991); GADD45A(1647); LRRC8C |
| | 913; 2244; | (84230); RAP1A(5906); SYCP1(6847); MDM4(4194); CCNL2(81669); |
| | | RCC2(55920); TESK2(10420); SASS6(163786); CTSS(1520); PYGO2(90780); |
| | | ASPM(259266); NUCKS1(64710); IL10(3586); BIRC6(57448); PRKCE |
| | | (5581); MSH2(4436); HK2(3099); RIF1(55183); E2F6(1876); RTN4(57142); |
| | | MCM6(4175); IFIH1(64135); TLK1(9874); FRZB(2487); NCKAP1(10787); |
| | | BARD1(580); CUL3(8452); FANCD2(2177); MLH1(4292); GNL3(26354); |
| | | APPL(26060); EAF2(55840); RAP2B(5912); TP73L(8626); OPA1(4976); |
| | | HD(3064); IL8(3576); NPNT(255743); FGF2(2247); PRKG2(5593); SLC7A11 |
| | | (23657); SPATA4(132851); SKP2(6502); CARD6(84674); LOC285636 |
| | | (285636); CCNB1(891); APC(324); G3BP(10146); BIRC1(4671); MCC(4163); |
| | | SEMA6A(57556); PPP2CA(5515); NME5(8382); CDC23(8697); CSNK1A1 |
| | | (1452); LMAN2(10960); TTK(7272); HECA(51696); DST(667); BCLAF1 |
| | | (9774); KATNA1(11104); PDCD2(5134); GLCCI1(113263); AHR(196); HDAC9 |
| | | (9734); VKORC1L1(154807); CLDN12(9069); RINT1(60561); BCAP29 |
| | | (55973); CUL1(8454); CARD4(10392); SFRP4(6424); CDK6(1021); ZC3HC1 |
| | | (51530); ESCO2(157570); UNC5D(137970); UBE2V2(7336); TERF1(7013); |
| | | E2F5(1875); PSD3(23362); DUSP4(1846); CCNE2(9134); EXT1(2131); |
| | | JAK2(3717); UNC13B(10497); INVS(27130); CDKN2B(1030); SEMA4D |
| | | (10507); PTCH(5727); CTNNAL1(8727); TSC1(7248); PTEN(5728); FAS |
| | | (355); HECTD2(143279); KIF11(3832); NOLC1(9221); MTG1(92170); TLL2 |
| | | (7093); C10orf46(143384); TIAL1(7073); HIPK3(10114); BIRC3(330); BIRC2 |
| | | (329); CUL5(8065); HRASLS3(11145); CDCA5(113130):PPP2R1B(5519); |
| | | MDM2(4193); APAF1(317); FZD10(11211); TNFRSF1A(7132); LRP6 |
| | | (4040); CBX5(23468); DDIT3(1649); PPP1CC(5501); NOS1(4842); ANAPC5 |
| | | (51433); CHFR(55743); BRCA2(675); TFDP1(7027); LATS2(26524); CSNK1A1L |
| | | (122011); PNN(5411); PPM1A(5494); MNAT1(4331); VASH1(22846); |
| | | NRXN3(9369); TRAF3(7187); DLG7(9787); NEK9(91754); PPP1R13B |
| | | (23368); BUB1B(701); MAPK6(5597); KIF23(9493); AVEN(57099); DAPK2 |
| | | (23604); TRAF7(84231); DNAJA3(9093); CTCF(10664); GSPT1(2935); |
| | | PAFAH1B1(5048); NLK(51701); NF1(4763); TADA2L(6871); CDC6(990); |
| | | MAPT(4137); CROP(51747); TLK2(11011); PRKCA(5578); GAS7(8522); BRCA1 |
| | | (672); MALT1(10892); ESCO1(114799); APOE(348); SPHK2(56848); |
| | | MCM8(84515); PLCB1(23236); CD40(958); PARD6B(84612); USP16(10600); |
| | | SON(6651); BRWD1(54014); SNF1LK(150094); YWHAH(7533); SEPT3 |
| | | (55964); MAPK1(5594); PPM1F(9647); MN1(4330); SMC1L2(27127); STAG2 |
| | | (10735); PHF6(84295); FIGF(2277); SMC1L1(8243); BTK(695); LAMP2 |
| | | (3920); CETN2(1069); |
| hsa-miR-324-3p | 66; 746; 747; | SESN2(83667); RBBP4(5928); KIAA1522(57648); UTP11L(51118); MACF1 |
| | | (23499); ST3GAL3(6487); GADD45A(1647); CDC14A(8556); PROK1(84432); |
| | | BCL9(607); PEA15(8682); FASLG(356); C1orf21(81563); RGS2(5997); |
| | | MDM4(4194); IL19(29949); IL24(11009); DISC1(27185); TNFRSF18(8784); |
| | | TNFRSF9(3604); DFFA(1676); FRAP1(2475); MAD2L2(10459); ZBTB17 |
| | | (7709); E2F2(1870); FAF1(11124); ITGB3BP(23421); NOTCH2(4853); |
| | | MCL1(4170); PKLR(5313); SNX17(9784); BIRC6(57448); PRKCE(5581); |
| | | HTRA2(27429); HK2(3099); BCL2L11(10018); PROC(5624); RIF1(55183); |
| | | PMS1(5378); CFLAR(8837); CD28(940); PTMA(5757); BOK(666); YWHAQ |
| | | (10971); CGREF1(10669); CCT4(10575); TGFA(7039); BUB1(699); ANAPC1 |
| | | (64682); BIN1(274); IFIH1(64135); BARD1(580); PAX3(5077); HDAC4 |
| | | (9759); DTYMK(1841); PCAF(8850); PDCD6IP(10015); CTNNB1(1499); |
| | | DAG1(1605); RBM5(10181); PLXNA1(5361); KLHDC6(166348); NEK11 |
| | | (79858); RNF7(9616); SMC4L1(10051); FXR1(8087); PSMD2(5708); CHRD |
| | | (8646); SENP2(59343); OPA1(4976); TADA3L(10474); MRPS25(64432); |
| | | TMEM110(375346); STAG1(10274); TFDP2(7029); SH3BP2(6452); HD(3064); |
| | | HCAP-G(64151); EREG(2069); CCNG2(901); NFKB1(4790); GAK(2580); |
| | | APBB2(323); NEK1(4750); KIAA1909(153478); FAM105B(90268); |
| | | NIPBL(25836); RAD17(5884); F2R(2149); APC(324); CSNK1G3(1456); R |

Table 4-37

| | | |
|---|---|---|
| | | AD50(10111); GALNT10(55568); ERGIC1(57222); PRLR(5618); GDNF(2668); |
| | | RPL37(6167); ATG12(9140); SEMA6A(57556); PPP2CA(5515); CDC23 |
| | | (8697); PFDN1(5201); HDAC3(8841); CSNK1A1(1452); DUSP1(1843); |
| | | FAM44B(91272); RNF130(55819); MAPK13(5603); IL17(3605); PTP4A1 |
| | | (7803); HECA(51696); DHX16(8449); CUL7(9820); TNFRSF21(27242); DST |
| | | (667); LATS1(9113); HDAC9(9734); SEPT7(989); EGFR(1956); FZD9(8326); |
| | | STAG3(10734); RINT1(60561); BCAP29(55973); CASP2(835); MAD1L1 |
| | | (8379); PMS2(5395); CARD4(10392); ELMO1(9844); SFRP4(6424); INHBA |
| | | (3624); IGFBP3(3486); PMS2L2(5380); CDK6(1021); MCM7(4176); FIS1 |
| | | (51024); ZC3HC1(51530); PTN(5764); BRAF(673); CLN8(2055); TNFRSF10C |
| | | (8794); FZD3(7976); UNC5D(137970); TACC1(6867); UBE2V2(7336); |
| | | FZD6(8323); LZTS1(11178); BIN3(55909); TNFRSF10B(8795); TNFRSF10A |
| | | (8797); ADRA1A(148); CLU(1191); DUSP4(1846); ZNF704(619279); |
| | | RAD21(5885); FBXO32(114907); FAM84B(157638); ANKRD15(23189); |
| | | CDC37L1(55664); JAK2(3717); UNC13B(10497); RECK(8434); C9orf47(286223); |
| | | INVS(27130); SLC25A25(114789); ABL1(25); RXRA(6256); CDKN2B |
| | | (1030); TAF1L(138474); C9orf25(203259); CD72(971); SEMA4D(10507); |
| | | CDC14B(8555); TRAF1(7185); TSC1(7248); CARD9(64170); ZMYND11 |
| | | (10771); SGPL1(8879); UNC5B(219699); LZTS2(84445); NOLC1(9221); |
| | | SUFU(51684); PDCD11(22984); IL2RA(3559); DLG5(9231); FBXW4(6468); |
| | | SMNDC1(10285); DCLRE1A(9937); TIAL1(7073); MKI67(4288); BNIP3 |
| | | (664); API5(8539); EXT2(2132); DAK(26007); INCENP(3619); DPF2(5977); |
| | | SIPA1(6494); LRP5(4041); CCND1(595); FADD(8772); ATM(472); CD3E |
| | | (916); CHEK1(1111); JAM3(83700); IGF2(3481); APBB1(322); MAP3K11 |
| | | (4296); PPP1CA(5499); FGF4(2249); FGF3(2248); FZD4(8322); SESN3(143686); |
| | | CASP1(834); BCL9L(283149); H2AFX(3014); FKBP4(2288); TNFRSF7 |
| | | (939); CNAP1(9918); LRRC23(10233); TEGT(7009); ESPL1(9700); |
| | | MDM2(4193); APAF1(317); KNTC1(9735); RAN(5901); NOL1(4839); LRP6 |
| | | (4040); KRAS(3845); HDAC7A(51564); ARF3(377); MCRS1(10445); FAIM2 |
| | | (23017); C12orf22(81566); ANAPC7(51434); CHFR(55743); BRCA2(675); |
| | | RFP2(10206); FLJ40296(122183); ING1(3621); FOXO1A(2308); PSEN1 |
| | | (5663); VASH1(22846); CALM1(801); OTUB2(78990); TRAF3(7187); SIVA |
| | | (10572); ACIN1(22985); EGLN3(112399); RAB15(376267); LTBP2(4053); |
| | | PGF(5228); CHES1(1112); AKT1(207); JAG2(3714); BRF1(2972); SLC12A6 |
| | | (9990); BMF(90427); PYGO1(26108); CSNK1G1(53944); PDCD7(10081); |
| | | TBC1D2B(23102); TSC2(7249); TRAF7(84231); MGRN1(23295); UBN1 |
| | | (29855); CARD15(64127); E2F4(1874); ELMO3(79767); AXIN1(8312); |
| | | GSPT1(2935); ERN2(10595); MAPK3(5595); PYCARD(29108); CLAPIN1 |
| | | (57019); TRADD(8717); SLC12A4(6560); SMPD3(55512); SLC7A5(8140); |
| | | MAPK7(5598); TRAF4(9618); NF1(4763); RAB11FIP4(84440); STARD3 |
| | | (10948); MAPT(4137); MYST2(11143); NME1(4830); TLK2(11011); PRKCA |
| | | (5578); MAP2K6(5608); SLC9A3R1(9368); TMEM104(54868); SPHK1(8877); |
| | | BIRC5(332); MNT(4335); P2RX1(5023); GAS7(8522); HCP1(113235); |
| | | LOC116236(116236); GAS2L2(246176); TCF2(6928); BECN1(8678); BRCA1 |
| | | (672); HDAC5(10014); PTRH2(51651); ERN1(2081); NPTX1(4884); CIDEA |
| | | (1149); MAPRE2(10982); MAPK4(5596); DCC(1630); MALT1(10892); |
| | | BCL2(596); FZR1(51343); CHAF1A(10036); PIN1(5300); DNM2(1785); |
| | | LOC126536(126536); PIK3R2(5296); PSMD8(5714); NFKBIB(4793); MAP3K10 |
| | | (4294); AXL(558); ARHGEF1(9138); BCL3(602); APOE(348); FOSB |
| | | (2354); SPHK2(56848); PPP2R1A(5518); ZNF443(10224); PRDX2(7001); |
| | | DPF1(8193); TGFB1(7040); DEDD2(162989); ERF(2077); ERCC2(2068); |
| | | PPP1R13L(10848); BBC3(27113); LIG1(3978); MAPRE1(22919); CEP250 |
| | | (11190); CTNNBL1(56259); C20orf11(54994); E2F1(1869); AURKA(6790); |
| | | DIDO1(11083); CHRNA4(1137); USP16(10600); BTG3(10950); ITGB2(3689); |
| | | BCL2L13(23786); SEPT5(5413); ADORA2A(135); GAS2L1(10634); |
| | | NF2(4771); SYNGR1(9145); EP300(2033); SEPT3(55964); GTSE1(51512); |
| | | DGCR2(9993); MAPK1(5594); PPM1F(9647); MN1(4330); CARD10(29775); |
| | | TOB2(10766); NHP2L1(4809); MAPK12(6300); PCTK1(5127); CCNB3 |
| | | (85417); TAF1(6872); BIRC4(331); AFF2(2334); IKBKG(8517); ARSD(414); |
| | | SMC1L1(8243); LAMP2(3920); HCFC1(3054); |
| hsa-miR-324-5p | 67; 748; 749; | PRDM16(63976); TP73(7161); KIF1B(23095); AGTRAP(57085); TNFRSF1B |
| | | (7133); NBL1(4681); SESN2(83667); RBBP4(5928); GADD45A(1647); |
| | | VANGL1(81839); BCL9(607); PBX1(5087); ADORA1(134); PRELP(5549); |
| | | IL24(11009); TGFB2(7042); C1orf95(375057); FRAP1(2475); MAD2L2(10459); |
| | | PADI2(11240); WASF2(10163); POU3F1(5453); SASS6(163786); PY |

Table 4-38

| | | |
|---|---|---|
| | | GO2(90780); RNASEL(6041); ASPM(259266); KIF21B(23046); C1orf157 |
| | | (284573); TP53BP2(7159); FH(2271); LOC165186(165186); BIRC6(57448); |
| | | PRKCE(5581); BRRN1(23397); ZAK(51776); CASP10(843); COL4A3(1285); |
| | | YWHAQ(10971); CGREF1(10669); CYP26B1(56603); PAX8(7849); FRZB |
| | | (2487); RBM15B(29890); APPL(26060); ADPRH(141); MCM2(4171); PSMD2 |
| | | (5708); OPA1(4976); FAM43A(131583); AXUD1(64651); CAMKV(79012); |
| | | SEMA3G(56920); ATR(545); HD(3064); CD38(952); HCAP-G(64151); |
| | | AFF1(4299); TLR2(7097); LETM1(3954); UNC5C(8633); DKK2(27123); |
| | | LEF1(51176); NIPBL(25836); LOC285636(285636); RAD17(5884); RAD50 |
| | | (10111); ERGIC1(57222); BNIP1(662); DAP(1611); PRLR(5618); HDAC3 |
| | | (8841); ARSI(340075); RNF44(22838); SIRT5(23408); FLJ45422(441140); |
| | | AIF1(199); CSNK2B(1460); MAPK13(5603); C6orf69(222658); PPP2R5D |
| | | (5528); PARC(23113); PTP4A1(7803); FOXO3A(2309); HECA(51696); SASH1 |
| | | (23328); ZDHHC14(79683); DHX16(8449); MDC1(9656); CCND3(896); |
| | | CUL7(9820); SESN1(27244); MAP3K5(4217); ZNRF2(223082); BCAP29 |
| | | (55973); CALN1(83698); CDK6(1021); KIAA0773(9715); RHOBTB2(23221); |
| | | TERF1(7013); FAM83A(84985); TNFRSF10D(8793); TNFRSF10A(8797); |
| | | CLU(1191); KIF13B(23303); RNF170(81790); LYNX1(66004); DAPK1 |
| | | (1612); C9orf47(286223); SYK(6850); TAF1L(138474); SURF5(6837); CARD9 |
| | | (64170); UNC5B(219699); BTRC(8945); SUFU(51684); PDCD11(22984); |
| | | TCF7L2(6934); BAG3(9531); PARD3(56288); C10orf54(64115); C10orf46 |
| | | (143384); BNIP3(664); API5(8539); EXT2(2132); F2(2147); ZFP91(80829); |
| | | MRPL49(740); RPS6KB2(6199); CUL5(8065); ATM(472); ABCG4(64137); |
| | | FLJ45300(399957); LOH11CR2A(4013); HRASLS3(11145); MAP3K11 |
| | | (4296); PTMS(5763); SP1(6667); METTL7B(196410); APAF1(317); RAN(5901); |
| | | LALBA(3906); FRBP11(51303); FAIM2(23017); PAWR(5074); ANAPC7 |
| | | (51434); HRK(8739); DIABLO(56616); SCARB1(949); CHFR(55743); HSMPP8 |
| | | (54737); RFP2(10206); FLJ40296(122183); TFDP1(7027); FOXO1A |
| | | (2308); NEK3(4752); LOC283487(283487); VASH1(22846); KIAA0125(9834); |
| | | EGLN3(112399); LTBP2(4053); PGF(5228); NEK9(91754); AKT1(207); |
| | | JAG2(3714); FGF7(2252); AQP9(366); TLN2(83660); C15orf39(56905); |
| | | SCAND2(54581); AVEN(57099); SLC12A6(9990); WDR72(256764); PDCD7 |
| | | (10081); MAPK8IP3(23162); KREMEN2(79412); DNASE1(1773); NDE1 |
| | | (54820); CYLD(1540); NOL3(8996); KIAA0513(9764); AXIN1(8312); ZNF500 |
| | | (26048); GRIN2A(2903); ERN2(10595); DNAJA2(10294); CIAPIN1 |
| | | (57019); CMTM4(146223); TXNL4B(54957); ALOX15B(247); MAPK7(5598); |
| | | IGF2BP1(10642); NGFR(4804); PPM1D(8493); PRKCA(5578); CARD14 |
| | | (79092); CARKL(23729); CAMKK1(84254); NALP1(22861); SPAG5(10615); |
| | | HDAC5(10014); SEPT4(5414); PTRH2(51651); ERN1(2081); SOCS3 |
| | | (9021); USP36(57602); TBC1D16(125058); CSNK1D(1453); EMILIN2(84034); |
| | | CIDEA(1149); BCL2(596); FLJ22709(79629); CCNE1(898); AXL(558); |
| | | ARHGEF1(9138); SPHK2(56848); ATF5(22809); ZNF324(25799); DAPK3 |
| | | (1613); UBXD1(80700); SIRT2(22933); DEDD2(162989); PLCB1(23236); |
| | | CEP250(11190); OPRL1(4987); CSNK2A1(1457); GGTL3(2686); RBL1 |
| | | (5933); CABLES2(81928); USP16(10600); AGPAT3(56894); APP(351); S100B |
| | | (6285); SEPT5(5413); SLC5A1(6523); MGAT3(4248); EP300(2033); ZC3H7B |
| | | (23264); SEPT3(55964); FLJ21125(79680); MAPK1(5594); PPM1F |
| | | (9647); RBM9(23543); DMC1(11144); POLDIP3(84271); C22orf9(23313); |
| | | SMC1L2(27127); XG(7499); PCTK1(5127); HDAC6(10013); TAF1(6872); |
| | | RP6-213H19.1(51765); AFF2(2334); IKBKG(8517); SMC1L1(8243); ARHGEF9 |
| | | (23229); FAM58A(92002); MTCP1(4515); |
| hsa-miR-329 | 68; 750; 751; | TNFRSF1B(7133); UBXD3(127733); CDC42(998); LIN28(79727); SFN(2810); |
| | 914; 915; | SLC6A17(388662); RAP1A(5906); VANGL1(81839); BCL9(607); IGSF4B |
| | | (57863); PEA15(8682); FASLG(356); QSCN6(5768); DFFA(1676); BCL10 |
| | | (8915); NOTCH2(4853); RP5-998N21.6(440686); ENSA(2029); MEF2D |
| | | (4209); DEDD(9191); PRKCE(5581); BRRN1(23397); CASP10(843); SEPT2 |
| | | (4735); E2F6(1876); CGREF1(10669); CARD12(58484); TGFA(7039); |
| | | MGAT4A(11320); MCM6(4175); EPHA4(2043); PAX3(5077); HDAC4(9759); |
| | | VHL(7428); PDCD6IP(10015); ACPP(55); RNF7(9616); PSMD2(5708); |
| | | SENP2(59343); RAF1(5894); CAMKV(79012); PROK2(60675); SIAH2(6478); |
| | | TNFSF10(8743); WHSC1(7468); HD(3064); PLK4(10733); TLR2(7097); |
| | | ABLIM2(84448); BTC(685); MAP2K1IP1(8649); DKK2(27123); SFRP2 |
| | | (6423); NEK1(4750); VEGFC(7424); PDCD6(10016); FAM105B(90268); CSNK1G3 |
| | | (1456); RAD50(10111); TCF7(6932); PCDHB9(56127); ERGIC1 |
| | | (57222); N4BP3(23138); PRLR(5618); KENAE(202243); SEMA6A(57556); |

Table 4-39

| | | |
|---|---|---|
| | | NRG2(9542); CD74(972); FBXW11(23291); LTA(4049); CSNK2B(1460); CDKN1A |
| | | (1026); FOXO3A(2309); C6orf71(389434); ATXN1(6310); NUDT3 |
| | | (11165); TNFRSF21(27242); BCLAF1(9774); AGPAT4(56895); PDCD2(5134); |
| | | FOXK1(221937); EGFR(1956); FZD1(8321); CASP2(835); MAD1L1(8379); |
| | | SOSTDC1(25928); IGFBP3(3486); ECOP(81552); MCM7(4176); MTPN |
| | | (136319); FASTK(10922); UBE2V2(7336); BIN3(55909); TNFRSF10D |
| | | (8793); TNFRSF10A(8797); CLU(1191); DUSP4(1846); SFRP1(6422); TP53INP1 |
| | | (94241); UNC13B(10497); DAPK1(1612); C9orf47(286223); FGD3 |
| | | (89846); FREQ(23413); ABL1(25); SEMA4D(10507); PTCH(5727); TSC1(7248); |
| | | CARD9(64170); BTRC(8945); PDCD11(22984); DOCK1(1793); CUL2 |
| | | (8453); ZWINT(11130); SMNDC1(10285); DCLRE1A(9937); ABLIM1(3983); |
| | | HIPK3(10114); RPS6KB2(6199); LOH11CR2A(4013); CHEK1(1111); |
| | | SIRT3(23410); IGF2(3481); DKK3(27122); RELA(5970); FGF4(2249); TEGT |
| | | (7009); PA2G4(5036); SOCS2(8835); CDCA3(83461); ARF3(377); FAIM2 |
| | | (23017); C12orf22(81566); AKAP11(11215); ING1(3621); LATS2(26524); |
| | | KIAA0323(23351); PSEN1(5663); VASH1(22846); TRAF3(7187); WDR22 |
| | | (8816); ERH(2079); DPF3(8110); LTBP2(4053); KLF13(51621); IGF1R(3480); |
| | | BMF(90427); CSNK1G1(53944); PDCD7(10081); HSPC065(29070); P8 |
| | | (26471); STX1B2(112755); DNAJA2(10294); SIAH1(6477); CIAPIN1(57019); |
| | | NLGN2(57555); MAPK7(5598); NLK(51701); NF1(4763); TADA2L(6871); |
| | | FZD2(2535); MAPT(4137); CROP(51747); PPM1D(8493); TLK2(11011); |
| | | SPHK1(8877); CARD14(79092); ANAPC11(51529); PHB(5245); MPO |
| | | (4353); ERN1(2081); USP36(57602); CSNK1D(1453); CABLES1(91768); MAPK4 |
| | | (5596); SMAD4(4089); MALT1(10892); PHLPP(23239); TXNL4A(10907); |
| | | FSTL3(10272); UBA52(7311); AXL(558); APOE(348); FOSB(2354); |
| | | GRLF1(2909); CDKN2D(1032); AKT2(208); LIG1(3978); CARD8(22900); |
| | | STK4(6789); DNAJC5(80331); BAK1(578); E2F1(1869); ELMO2(63916); |
| | | B4GALT5(9334); UBE2V1(7335); CABLES2(81928); DIDO1(11083); BTBD4 |
| | | (140685); CHAF1B(8208); TSSK2(23617); NF2(4771); MCM5(4174); TNRC6B |
| | | (23112); TEF(7008); MAPK8IP2(23542); MAPK1(5594); PPM1F |
| | | (9647); MN1(4330); PDGFB(5155); TOB2(10766); HDAC6(10013); BIRC4 |
| | | (331); BTK(695); SEPT6(23157); ARHGEF6(9459); BCAP31(10134); |
| hsa-miR-331 | 69; 752; 753; | TNFRSF1B(7133); PAX7(5081); LDLRAD2(401944); RCC1(1104); UTP11L |
| | 2245; | (51118); MACF1(23499); NFYC(4802); PLK3(1263); CDEN2C(1031); CDC14A |
| | | (8556); RAP1A(5906); BCL9(607); ADORA1(134); IL19(29949); CIAS1 |
| | | (114548); TNFRSF18(8784); CDC2L2(985); CASP9(842); RCC2(55920); |
| | | YARS(8565); TESK2(10420); BCL10(8915); NOTCH2(4853); PYGO2(90780); |
| | | DEDD(9191); MAPRE3(22924); MSH2(4436); BCL2L11(10018); RIF1 |
| | | (55183); ZAK(51776); CASP10(843); CASP8(841); ALS2CR19(117583); INHA |
| | | (3623); COL4A3(1285); CGREF1(10669); ZNF513(130557); EDAR(10913); |
| | | BIN1(274); TLK1(9874); FZD5(7855); CUL3(8452); HDAC4(9759); VHL |
| | | (7428); MCM2(4171); FAIM(55179); PSMD2(5708); TP73L(8626); SRGAP3 |
| | | (9901); RAF1(5894); AXUD1(64651); GSK3B(2932); PIK3CB(5291); SIAH2 |
| | | (6478); TNFSF10(8743); WHSC1(7468); FGF2(2247); GAK(2580); DKK2 |
| | | (27123); VEGFC(7424); APC(324); TCF7(6932); CPLX2(10814); UNC5A |
| | | (90249); SEMA6A(57556); IRF1(3659); CDC25C(995); CAMK2A(815); |
| | | CD74(972); FBXW11(23291); DBN1(1627); RIPK1(8737); TCF19(6941); LTA |
| | | (4049); TNF(7124); CSNK2B(1460); MAPK13(5603); VEGF(7422); FOXO3A |
| | | (2309); HECA(51696); DHX16(8449); MDC1(9656); DAXX(1616); BAK1 |
| | | (578); IHPK3(117283); CUL7(9820); DST(667); FRK(2444); TTYH3(80727); |
| | | EGFR(1956); FZD9(8326); LOC441257(441257); FZD1(8321); BCAP29 |
| | | (55973); LEP(3952); TRIAD3(54476); PMS2(5395); CARD4(10392); INHBA |
| | | (3624); IGFBP3(3486); YWHAG(7532); EPHA1(2041); PTK2B(2185); |
| | | SULF1(23213); WISP1(8840); LZTS1(11178); BIN3(55909); TNFRSF10A |
| | | (8797); TRIM35(23087); CLU(1191); SFRP1(6422); MTSS1(9788); ANKRD15 |
| | | (23189); UNC13B(10497); DAPK1(1612); ARL1(25); CDKN2A(1029); |
| | | CDKN2B(1030); BAG1(573); APBA1(320); GAS1(2619); TNFSF15(9966); |
| | | TRAF1(7185); CIZ1(25792); TSC1(7248); CARD9(64170); SIRT1(23411); |
| | | SGPL1(8879); LZTS2(84445); SUFU(51684); PDCD11(22984); CSPG6 |
| | | (9126); BAG3(9531); DLG5(9231); SFRP5(6425); FBXW4(6468); C10orf46 |
| | | (143384); STIM1(6786); WEE1(7465); EXT2(2132); TSFAN18(90139); F2 |
| | | (2147); C11orf11(747); DPF2(5977); SSSCA1(10534); SIPA1(6494); RPS6KB2 |
| | | (6199); CDKN1C(1028); RHOG(391); DKK3(27122); BAD(572); HSPC152 |
| | | (51504); CDCA5(113130); MAP3K11(4296); BRMS1(25855); PPP1CA |
| | | (5499); FZD4(8322); PPP2R1B(5519); BCL9L(283149); TNFRBF7(939); |

Table 4-40

| | | |
|---|---|---|
| | | CNAP1(9918); PTMS(5763); BCL2L14(79370); TENC1(23371); ESPL1(9700); |
| | | PDE1B(5153); RBMS2(5939); SOCS2(8835); CRADD(8738); APAF1 |
| | | (317); TPCN1(53373); NOL1(4839); ING4(51147); BCAT1(586); HDAC7A |
| | | (51564); MCRS1(10445); DIP13B(55198); HRK(8739); FGF9(2254); FOXO1A |
| | | (2308); PSEN1(5663); VASH1(22846); TRAF3(7187); KIAA0125(9834); |
| | | ACIN1(22985); LTBP2(4053); PGF(5228); TGFB3(7043); BAG5(9529); PPP1R13B |
| | | (23368); AKT1(207); JAG2(3714); KIF23(9493); IGF1R(3480); BMF |
| | | (90427); DAPK2(23604); TSC2(7249); TRAF7(84231); KREMEN2(79412); |
| | | DNASE1(1773); RBL2(5934); ELMO3(79767); CTCF(10664); NME3 |
| | | (4832); PKMYT1(9088); GSPT1(2935); ERN2(10595); MAPK3(5595); TRADD |
| | | (8717); SLC12A4(6560); TXNL4B(54957); MPHOSPH6(10200); PCOLN3 |
| | | (5119); ALOX12(239); ALOX15B(247); MAPK7(5598); CDC6(990); MAPT |
| | | (4137); CROP(51747); NME2(4831); TLK2(11011); PRKCA(5578); CARD14 |
| | | (79092); BAIAP2(10458); GPS1(2873); MNT(4335); P2RX1(5023); TP53 |
| | | (7157); GAS7(8522); GAS2L2(246176); CACNB1(782); BECN1(8678); |
| | | HDAC5(10014); SEPT4(5414); ERN1(2081); SOCS3(9021); CBX4(8535); |
| | | TBC1D16(125058); PCYT2(5833); MAFG(4097); CABLES1(91768); PHLPP |
| | | (23239); SERPINB2(5055); PARD6G(84552); CSNK1G2(1455); FZR1 |
| | | (51343); CHAF1A(10036); MAP2K7(5609); PIN1(5300); BTBD14B(112939); |
| | | RAB8A(4218); PIK3R2(5296); MAP3K10(4294); AXL(558); ARHGEF1 |
| | | (9138); FOSB(2354); SPHK2(56848); BAX(581); ATF5(22809); HLRC1(83475); |
| | | MATK(4145); CDKN2D(1032); DPF1(8193); TGFB1(7040); ERF(2077); |
| | | BBC3(27113); LIG1(3978); CDC25B(994); MYBL2(4605); BIRC7(79444); |
| | | CSNK2A1(1457); BCL2L1(598); E2F1(1869); RIMS4(140730); DIDO1(11083); |
| | | USP16(10600); CDC45L(8318); ADORA2A(135); NF2(4771); SYNGR1 |
| | | (9145); EP300(2033); MAPK8IP2(23542); BID(637); MAPK1(5594); PPM1F |
| | | (9647); MN1(4330); CARD10(29775); SBF1(6305); PCTK1(5127); HDAC6 |
| | | (10013); TAF1(6872); P2RY8(286530); FIGF(2277); SMC1L1(8243); PDCD8 |
| | | (9131); HCFC1(3054); MTCP1(4515); |
| hsa-miR-337 | 70; | PRKCZ(5590); DFFB(1677); PAX7(5081); NBL1(4681); NUDC(10726); SESN2 |
| | | (83667); MACF1(23499); NFYC(4802); PLK3(1263); CDC7(8317); CDC14A |
| | | (8556); SYCP1(6847); CD2(914); BNIPL(149428); PEA15(8682); FASLG |
| | | (356); IL24(11009); CIAS1(114548); CDC2L2(985); TNFRSF14(8764); |
| | | TNFRSF9(3604); CTNNBIP1(56998); FRAP1(2475); CASP9(842); ZBTB17 |
| | | (7709); RCC2(55920); E2F2(1870); TESK2(10420); MCL1(4170); S100A6 |
| | | (6277); PYGO2(90780); CD5L(922); DEDD(9191); RHOB(388); PPP1CB |
| | | (5500); BIRC6(57448); PRKCE(5581); MSH2(4436); HTRA2(27429); HK2 |
| | | (3099); TCF7L1(83439); KCNIP3(30818); BRRN1(23397); PDCL3(79031); |
| | | BCL2L11(10018); PROC(5624); RIF1(55183); CASP8(841); CD28(940); ALS2CR19 |
| | | (117583); INHA(3623); PSMD1(5707); BOK(666); CGREF1(10669); |
| | | PPM1G(5496); RTN4(57142); TIA1(7072); EDAR(10913); ANAPC1(64682); |
| | | BIN1(274); IFIH1(64135); FZD5(7855); BARD1(580); HDAC4(9759); |
| | | DTYMK(1841); FANCD2(2177); DLEC1(9940); ZNF651(92999); RBM5 |
| | | (10181); GNL3(26354); PCNP(57092); MCM2(4171); ACPP(55); RNF7(9616); |
| | | PSMD2(5708); CHRD(8646); SENP2(59343); KNG1(3827); TADA3L(10474); |
| | | CIDEC(63924); AXUD1(64651); RASSF1(11186); TUSC4(10641); |
| | | NEK4(6787); DNASE1L3(1776); UBE1C(9039); STAG1(10274); TFDP2(7029); |
| | | ATR(545); HD(3064); CD38(952); NFKB1(4790); FGF2(2247); TLR2 |
| | | (7097); FAM53A(152877); APBB2(323); PRKG2(5593); UNC5C(8633); DKK2 |
| | | (27123); LEF1(51176); SFRP2(6423); NEK1(4750); VEGFC(7424); SKP2 |
| | | (6502); CARD6(84674); TCF7(6932); UNC5A(90249); MAML1(9794); PRLR |
| | | (5618); BIRC1(4671); MCC(4163); SEMA6A(57556); IRF1(3659); PPP2CA |
| | | (5515); CDC23(8697); CDC25C(995); CD14(929); HDAC3(8841); FGF1 |
| | | (2246); CSNK1A1(1452); CD74(972); FBXW11(23291); DDX41(51428); |
| | | TCF19(6941); TNF(7124); PACSIN1(29993); MAPK13(5603); CDKN1A(1026); |
| | | PARC(23113); CDC5L(988); BAG2(9532); FOXO3A(2309); TNFAIP3 |
| | | (7128); NEDD9(4739); DHX16(8449); MDC1(9656); CUL7(9820); MCM3 |
| | | (4172); DST(667); MAP3K5(4217); LATS1(9113); PDCD2(5134); AHR(196); |
| | | HDAC9(9734); STK17A(9263); FZD1(8321); STAG3(10734); BCAP29(55973); |
| | | CUL1(8454); MAD1L1(8379); TRIAD3(54476); SOSTDC1(25928); CARD4 |
| | | (10392); ELMO1(9844); SFRP4(6424); IGFBP3(3486); TNS3(64759); |
| | | FIS1(51024); ZC3HC1(51530); HIPK2(28996); FASTK(10922); POLR3D |
| | | (661); TNFRSF10C(8794); PTK2B(2185); UNC5D(137970); TACC1(6867); |
| | | SULF1(23213); WISP1(8840); LZTS1(11178); BIN3(55909); TNFRSF10D |
| | | (8793); TNFRSF10A(8797); TRIM35(23087); DUSP4(1846); DKK4(271 |

Table 4-41

| | | |
|---|---|---|
| | | 21); MOS(4342); TP53INP1(94241); STK3(6788); MTSS1(9788); ANKRD15 |
| | | (23189); RECK(8434); DAPK1(1612); INVS(27130); CDK9(1025); ABL1 |
| | | (25); CDKN2A(1029); FANCG(2189); GAS1(2619); SEMA4D(10507); TNFSF15 |
| | | (9966); TNFSF8(944); TRAF1(7185); TSC1(7248); CARD9(64170); ANAPC2 |
| | | (29882); ZMYND11(10771); C10orf9(219771); SIRT1(23411); UNC5B |
| | | (219699); FRAT1(10023); LZTS2(84445); SUFU(51684); PDCD11(22984); |
| | | NEURL(9148); TCF7L2(6934); DOCK1(1793); FZD8(8325); ZWINT |
| | | (11130); PPP3CB(5532); DLG5(9231); C10orf46(143384); TIAL1(7073); FAM53B |
| | | (9679); WEE1(7465); TMEM86A(144110); INCENP(3619); DPF2(5977); |
| | | SSSCA1(10534); SIPA1(6494); BIRC2(329); CUL5(8065); ATM(472); |
| | | LOH11CR2A(4013); EI24(9538); IGF2(3481); APBB1(322); EIF4G2(1982); |
| | | DKK3(27122); WT1(7490); HRASLS3(11145); CDCA5(113130); MAP3K11 |
| | | (4296); RELA(5970); CLCF1(23529); PPP1CA(5499); FGF4(2249); FGF3 |
| | | (2248); FZD4(8322); CASP1(834); PPP2R1B(5519); BCL9L(283149); H2AFX |
| | | (3014); CNAP1(9918); PTMS(5763); PTPN6(5777); ESPL1(9700); PA2G4 |
| | | (5036); CRADD(8738); KNTC1(9735); FZD10(11211); BCAT1(586); KRAS |
| | | (3845); HDAC7A(51564); MCRS1(10445); FAIM2(23017); C12orf22 |
| | | (81566); DDIT3(1649); DUSP6(1848); BTG1(694); DIP13B(55198); ANAPC7 |
| | | (51434); CDK2AP1(8099); TNFRSF19(55504); ING1(3621); CUL4A(8451); |
| | | TFDP1(7027); LATS2(26524); NEK3(4752); BCL2L2(599); MNAT1(4331); |
| | | VASH1(22846); TRAF3(7187); PACS2(23241); ACIN1(22985); CIDEB |
| | | (27141); STRN3(29966); NFKBIA(4792); RAB15(376267); NEK9(91754); |
| | | PPP1R13B(23368); JAG2(3714); MAPK6(5597); SCAMP5(192683); ALPK3 |
| | | (57538); IGF1R(3480); NDN(4692); AVEN(57099); BMF(90427); BCL2L10 |
| | | (10017); FLJ38723(255180); DAPK2(23604); TBC1D2B(23102); TSC2 |
| | | (7249); KREMEN2(79412); DNAJA3(9093); PLK1(5347); KIAA0556(23247); |
| | | CYLD(1540); KATNB1(10300); NOL3(8996); ELMO3(79767); CTCF |
| | | (10664); NFATC3(4775); CDT1(81620); NME3(4832); PRMYT1(9088); GSPT1 |
| | | (2935); ERN2(10595); P8(26471); MAPK3(5595); SIAH1(6477); FTS(64400); |
| | | CSNK2A2(1459); APPBP1(8883); TRADD(8717); SLC12A4(6560); |
| | | SMPD3(55512); TXNL4B(54957); HIC1(3090); PAFAH1B1(5048); GSG2 |
| | | (83903); ALOM12(239); TNFSF12(8742); ALOX15B(247); NTN1(9423); TRAF4 |
| | | (9618); LIG3(3980); CDC6(990); NGFR(4804); TLK2(11011); MRC2(9902); |
| | | CDK3(1018); SPHK1(8877); BIRC5(332); CARD14(79092); GPS1(2873); |
| | | P2RX1(5023); NALP1(22861); GAS7(8522); SPAG5(10615); GAS2L2 |
| | | (246176); BECN1(8678); BRCA1(672); HDAC5(10014); PHB(5245); MPO |
| | | (4353); ERN1(2081); SOCS3(9021); CSNK1D(1453); CETN1(1068); CIDEA |
| | | (1149); CABLES1(91768); RNF165(494470); MAPK4(5596); MALT1(10892); |
| | | PHLPP(23239); ESCO1(114799); BCL2(596); TXNL4A(10907); GADD45B |
| | | (4616); FZR1(51343); HMG20B(10362); CHAF1A(10036); UHRF1(29128); |
| | | TNFSF9(8744); DNM2(1785); CASP14(23581); PIK3R2(5296); UBA52 |
| | | (7311); PSMD8(5714); MAP3K10(4294); AXL(558); ARHGEF1(9138); |
| | | BCL3(602); APOE(348); FOSB(2354); SPHK2(56848); BAX(581); BCL2L12 |
| | | (83596); ATF5(22809); PPP2R1A(5518); NALP2(55655); AES(166); MATK |
| | | (4145); DAPK3(1613); TNFSF7(970); TNFSF14(8740); CDC37(11140); CDKN2D |
| | | (1032); DPF1(8193); SIRT2(22933); TGFB1(7040); DEDD2(162989); |
| | | ERF(2077); PPP1R13L(10848); LIG1(3978); CARD8(22900); TRIB3(57761); |
| | | MCM8(84515); PLCB1(23236); CEP250(11190); CTNNBL1(56259); |
| | | MYBL2(4605); STK4(6789); SLC12A5(57468); BIRC7(79444); PCNA(5111); |
| | | BCL2L1(598); SCAND1(51282); ELMO2(63916); CTCFL(140690); SYCP2 |
| | | (10388); CABLES2(81928); DIDO1(11083); USP16(10600); HUNK(30811); |
| | | SON(6651); MX1(4599); ITGB2(3689); S100B(6285); BCL2L13(23786); |
| | | CDC45L(8318); SEPT5(5413); SMARCB1(6598); ADORA2A(135); GAS2L1 |
| | | (10634); NF2(4771); MTP18(51537); YWHAH(7533); PGEA1(25776); |
| | | SEPT3(55964); GTSE1(51512); MAPK8IP2(23542); BID(637); MAPK1(5594); |
| | | PPM1F(9647); MN1(4330); CARD10(29775); DMC1(11144); PDGFB |
| | | (5155); TOB2(10766); SMC1L2(27127); MAPK12(6300); HDAC6(10013); |
| | | MLLT7(4303); BIRC4(331); RP6-213H19.1(51765); IKBKG(8517); PPP2R3B |
| | | (28227); P2RY8(286530); FIGF(2277); SMC1L1(8243); BTK(695); SEPT6 |
| | | (23157); PDCD8(9131); BCAP31(10134); HCFC1(3054); MECP2(4204); |
| hsa-miR-340 | 71; 2246; 2247; | DFFB(1677); HDAC1(3065); MACF1(23499); PLK3(1263); CDKN2C(1031); |
| | | CDC7(8317); RAP1A(5906); BNIPL(149428); ADORA1(134); MDM4(4194); |
| | | TRAF5(7188); CPSF3L(54973); CDC2L2(985); TNFRSF9(3604); CTNNBIP1 |
| | | (56998); FRAP1(2475); ZBTB17(7709); E2F2(1870); CLSPN(639 |

Table 4-42

| | | |
|---|---|---|
| | | (388); PPP1CB(5500); BCL2L11(10018); ZAK(51776); E2F6(1876); TGFA |
| | | (7039); IL1A(3552); BIN1(274); FRZB(2487); HDAC4(9759); PDCD6IP |
| | | (10015); CTNNB1(1499); TDGF1(6997); APPL(26060); RNF7(9616); TADA3L |
| | | (10474); MAGI1(9223); MCF2L2(23101); CD38(952); FGF5(2250); C4orf15 |
| | | (79441); SKP2(6502); NIPBL(25836); ERBB2IP(55914); PCDEB9 |
| | | (56127); PCDHB11(56125); DDX41(51428); MAPK13(5603); VEGF(7422); |
| | | FOXO3A(2309); TAPBP(6892); DAXX(1616); CUL7(9820); DST(667); TSPYL1 |
| | | (7259); PDCD2(5134); PMS2(5395); CYCS(54205); BNIP3L(665); TACC1 |
| | | (6867); UBE2V2(7336); SULF1(23213); TNFRSF10B(8795); ADRA1A |
| | | (148); TRIM35(23087); SFRP1(6422); ANKRD15(23189); UNC13B(10497); |
| | | DAPK1(1612); TAF1L(138474); PTCH(5727); ASB6(140459); TSC1(7248); |
| | | SGPL1(8879); UNC5B(219699); PTEN(5728); PDCD11(22984); BAG3 |
| | | (9531); DOCK1(1793); CUL2(8453); SYT15(83849); FRAT2(23401); FGF8 |
| | | (2253); C10orf46(143384); MKI67(4288); F2(2147); INCENP(3619); SSSCA1 |
| | | (10534); SIPA1(6494); RPS6KB2(6199); LRRC51(220074); ATM(472); |
| | | CDKN1C(1028); APBB1(322); CDCA5(113130); MAP3K11(4296); FZD4 |
| | | (8322); PPP2R1B(5519); PTPN6(5777); ESPL1(9700); APAF1(317); KRAS |
| | | (3845); HDAC7A(51564); FAIM2(23017); HSMPP8(54737); VASH1(22846); |
| | | TRAF3(7187); ACIN1(22985); PGF(5228); MOAP1(64112); BAG5(9529); |
| | | JAG2(3714); TSC2(7249); CCNF(899); DNAJA3(9093); PKMYT1(9088); |
| | | ERN2(10595); P8(26471); SEPT1(1731); FTS(6400); TERF2(7014); PNPO |
| | | (55163); TLK2(11011); PRKCA(5578); SLC9A3R1(9368); SPHK1(8877); |
| | | CARD14(79092); NALP1(22861); GAS7(8522); GAS2L2(246176); BRCA1 |
| | | (672); SOCS3(9021); TXNL4A(10907); CSNK1G2(1455); UHRF1(29128); |
| | | TNFSF9(8744); UBA52(7311); CCNE1(898); APLP1(333); APOE(348); SPHK2 |
| | | (56848); LOC390980(390980); MATK(4145); ZNF490(57474); DPF1 |
| | | (8193); TGFB1(7040); ERCC2(2068); LIG1(3978); STK4(6789); PARD6B |
| | | (84612); DIDO1(11083); CHAF1B(8208); APP(351); S100B(6285); GAS2L1 |
| | | (10634); NF2(4771); MAPK8IP2(23542); MAPK1(5594); SHOX(6473); TAF1 |
| | | (6872); BIRC4(331); LOC2D3547(203547); HCFC1(3054); |
| hsa-miR-342 | 72; 754; 2248; | PRKCZ(5590); DFFB(1677); NBL1(4681); CDC42(998); SESN2(83667); LCK |
| | 2249; | (3932); RBBP4(5928); CDC20(991); SH3GLB1(51100); RAP1A(5906); |
| | | BNIPL(149428); C1orf89(200205); CIAS1(114548); TNFRSF18(8784); CCNL2 |
| | | (81669); CTNNBIP1(56998); FRAP1(2475); RCC2(55920); YARS(8565); |
| | | BCL10(8915); NOTCH2(4853); PYGO2(90780); CD5L(922); TP53BP2 |
| | | (7159); BIRC6(57448); HTRA2(27429); HK2(3099); TCF7L1(83439); FLJ13910 |
| | | (64795); KCNIP3(30818); BRRN1(23397); PLEKHB2(55041); RIF1 |
| | | (55183); ZAK(51776); CFLAR(8837); CASP8(841); INHA(3623); BOK(666); |
| | | CGREF1(10669); CARD12(58484); EIF2AK2(5610); TIA1(7072); ERBB4 |
| | | (2066); CUL3(8452); PCAF(8850); NEK11(79858); FAIM(55179); AXUD1 |
| | | (64651); RASSF1(11186); TFDP2(7029); WHSC1(7468); HD(3064); EREG |
| | | (2069); SEPT11(55752); FGF2(2247); KIAA1727(85462); BTC(685); DKK2 |
| | | (27123); LEF1(51176); CASP6(839); CASP3(836); NIPBL(25836); UTP15 |
| | | (84135); PRLR(5618); MCC(4163); ATG12(9140); IRF1(3659); NRG2(9542); |
| | | CSNK1A1(1452); E2F3(1871); BTN2A1(11120); CDKN1A(1026); PARC |
| | | (23113); VEGF(7422); PNRC1(10957); SASH1(23328); IER3(8870); CUL7 |
| | | (9820); TRAM2(9697); DST(667); SGK(6446); PLAGL1(5325); PDCD2(5134); |
| | | HDAC9(9734); SEPT7(989); EGFR(1956); FZD1(8321); CASP2(835); |
| | | PMS2(5395); CYCS(54205); SFRP4(6424); INHBA(3624); ZNF117(7670); |
| | | YWHAG(7532); CDK6(1021); MCM7(4176); FIS1(51024); UBE2H(7328); |
| | | BRAF(673); CLN8(2055); POLR3D(661); TACC1(6867); ADRA1A(148); TRIM35 |
| | | (23087); DKK4(27121); TNFRSF11B(4982); ANKRD15(23189); UNC13B |
| | | (10497); DAPK1(1612); ABL1(25); TRAF2(7186); CCL19(6363); SEMA4D |
| | | (10507); PTCH(5727); TNFSF8(944); CARD9(64170); C10orf9(219771); |
| | | SIRT1(23411); SGPL1(8879); C10orf132(401647); LZTS2(84445); BTRC |
| | | (8945); SUFU(51684); PDCD11(22984); CUL2(8453); SPOCK2(9806); |
| | | DLG5(9231); SFRP5(6425); C10orf46(143384); MKI67(4288); EXT2(2132); |
| | | SIPA1(6494); RPS6KB2(6199); CCND1(595); FADD(8772); BIRC3(330); |
| | | BIRC2(329); ATM(472); CD3E(916); IGF2(3481); FZD4(8322); IL18(3606); |
| | | BCL9L(283149); CCND2(894); LTBR(4055); TNFRSF7(939); TEGT(7009); |
| | | ESPL1(9700); PA2G4(5036); KNTC1(9735); FAM101A(144347); TNFRSF1A |
| | | (7132); HDAC7A(51564); CDK4(1019); DUSP6(1848); PPP1CC(5501); |
| | | C12orf43(64897); CHFR(55743); RFP2(10206); ING1(3621); LATS2(26524); |
| | | NEK3(4752); BCL212(599); TRAF3(7187); SIVA(10572); KIAA012 |
| | | 5(9834); LTBP2(4053); BAG5(9529); PPP1R13B(23368); KLF13(51621); KIF23 |
| | | (9493); IGF1R(3480); NDN(4692); BCL2L10(10017); DAPK2(23604); |
| | | CHTF18(63922); TSC2(7249); CCNF(899); DNASE1(1773); DNAJA3(9093); |
| | | COQ7(10229); PLK1(5347); E2F4(1874); ELMO3(79767); CTCF(10664); |
| | | FLJ45121(400556); GSPT1(2935); EXOSC6(118460); NTN1(9423); LIG3 |
| | | (3980); TADA2L(6871); MLLT6(4302); LASP1(3927); CDC6(990); FZD2 |
| | | (2535); MAPT(4137); NGFR(4804); PPM1D(8493); P2RX1(5023); SPAG5(10615); |
| | | NUFIP2(57532); BRCA1(672); PHB(5245); SEPT4(5414); ERN1(2081); |
| | | SOCS3(9021); VAPA(9218); MAPK4(5596); BCL2(596); PIK3R2(5296); |
| | | APOE(348); FOSB(2354); GRLF1(2909); BAX(581); NALP2(55655); DIRAS1 |
| | | (148252); CDKN2D(1032); PRDX2(7001); LIG1(3978); KLK10(5655); |
| | | MCM8(84515); CEP250(11190); STK4(6789); PARD6B(84612); C20orf11 |
| | | (54994); CSNK2A1(1457); BCL2L1(598); E2F1(1869); RBL1(5933); WDR4 |
| | | (10785); ITGB2(3689); BCL2L13(23786); ADORA2A(135); YWHAH(7533); |
| | | MCM5(4174); PGEA1(25776); EP300(2033); SEPT3(55964); GTSE1(51512); |
| | | MAPK1(5594); PPM1F(9647); MN1(4330); CHEK2(11200); PDGFB |
| | | (5155); PCTK1(5127); IKBKG(8517); SMC1L1(8243); SEPT6(23157); FAM58A |
| | | (92002); |
| hsa-miR-346 | 73; 755; 756; | NBL1(4681); NUDC(10726); MACF1(23499); CDC20(991); PROK1(84432); |
| | | DAP3(7818); IL19(29949); IL24(11009); C1orf95(375057); C1orf69(200205); |
| | | CLAS1(114548); E2F2(1870); CLSPN(63967); MUTYH(4595); NOTCH2 |
| | | (4853); FH(2271); BIRC6(57448); PRKCE(5581); MSH2(4436); HTRA2 |
| | | (27429); HK2(3099); PTPN18(26469); RIF1(55183); ALS2CR19(117583); INHA |
| | | (3623); EDAR(10913); ANAPC1(64682); FRZB(2487); STAT1(6772); |
| | | FANCD2(2177); SLC6A6(6533); PCAF(8850); APPL(26060); CIDEC(63924); |
| | | RAF1(5894); AXUD1(64651); TRAIP(10293); RASSF1(11186); FHIT |
| | | (2272); PIK3CB(5291); GAK(2580); UNC5C(8633); NEK1(4750); SKP2(6502); |
| | | APC(324); CSNK1G3(1456); UNC5A(90249); DAP(1611); PRLR(5618); |
| | | MCC(4163); SEMA6A(57556); CDC23(8697); DUSP1(1843); DDX41(51428); |
| | | RNF130(55819); RIPK1(8737); CDKN1A(1026); CDC5L(988); SASH1 |
| | | (23328); MDC1(9656); IHPK3(117283); MCM3(4172); DST(667); FRK(2444); |
| | | LATS1(9113); DBNL(28988); LOC441257(441257); RINT1(60561); HBP1 |
| | | (26959); CUL1(8454); CYCS(54205); SFRP4(6424); CDK6(1021); MCM7 |
| | | (4176); PTN(5764); HIPK2(28996); PTK2B(2185); UBE2V2(7336); SULF1 |
| | | (23213); PINX1(54984); LZTS1(11178); BIN3(55909); ANKRD15(23189); |
| | | UNC13B(10497); CDK9(1025); TRAF2(7186); CDKN2B(1030); ANKS6 |
| | | (203286); CTNNAL1(8727); TSC1(7243); BAG3(9531); PPP3CB(5532); DLG5 |
| | | (9231); DCLRE1A(9937); FAM53B(9679); RPS6KB2(6199); LRP5(4041); |
| | | CCND1(595); HRAS(3265); DKK3(27122); PTH(5741); BAD(572); FGF3 |
| | | (2248); CASP4(837); CCND2(894); LTBR(4055); CNAP1(9918); ESPL1 |
| | | (9700); NOL1(4839); LRP6(4040); KRAS(3845); FAIM2(23017); DIP13B(55198); |
| | | BRCA2(675); TFDP1(7027); LATS2(26524); LIG4(3981); BCL2L2 |
| | | (599); TRAF3(7187); GZMB(3002); STRN3(29966); EGLN3(112399); CDKL1 |
| | | (8814); NIN(51199); ERH(2079); PPP1R13B(23368); FGF7(2252); CSK |
| | | (1445); BMF(90427); CSNK1G1(53944); TSC2(7249); TRAF7(84231); CCNF |
| | | (899); TNFRSF12A(51330); CARD15(64127); E2F4(1874); ELMO3(79767); |
| | | AXIN1(8312); PKMYT1(9088); SMPD3(55512); HIC1(3090); TNFSF12 |
| | | (8742); ALOX15B(247); TRAF4(9618); NF1(4763); LIG3(3980); MAPT |
| | | (4137); NME1(4830); SLC9A3R1(9368); CARD14(79092); MNT(4335); |
| | | SEPT4(5414); ERN1(2081); CSNK1D(1453); MAPRE2(10982); DCC(1630); PHLPP |
| | | (23239); ROCK1(6093); FZR1(51343); HMG20B(10362); CHAF1A |
| | | (10036); UHRF1(29128); PIN1(5300); DNM2(1785); APLP1(333); AXL(558); |
| | | FOSB(2354); BAX(581); MATK(4145); DNASE2(1777); MMP24(10893); |
| | | CEP250(11190); C20orf11(54994); CSNK2A1(1457); BCL2L1(598); APP |
| | | (351); BRWD1(54014); SNF1LK(150094); ITGB2(3689); BCL2L13(23786); |
| | | SEPT5(5413); RAB36(9609); GAS2L1(10634); CABP7(164633); MTP18(51537); |
| | | MCM5(4174); EP300(2033); GTSE1(51512); MAPK1(5594); MN1(4330); |
| | | PDGFB(5155); TOB2(10766); MAPK12(6300); MAGEH1(28986); |
| hsa-miR-362 | 74; 2250; | RCC1(1104); HDAC1(3065); S100PBP(64766); MACF1(23499); GADD45A |
| | | (1647); CDC14A(8556); PROK1(84432); BCL9(607); DAP3(7818); UHMK1 |
| | | (127933); DDR2(4921); NAV1(89796); IPO9(55705); PRELP(5549); ATP2B4 |
| | | (493); IL19(29949); C1orf107(27042); TRAF5(7188); TGFB2(7042); |
| | | SYNC1(81493); TRIM62(55223); CSMD2(114784); MTF1(4520); SASS6(163786); |
| | | MCL1(4170); PIK3C2B(5287); GREM2(64388); AKT3(10000); FLJ40869 |
| | | (348654); BIRC6(57448); MSH2(4436); HK2(3099); KCNIP3(3081 |

Table 4-44

| | | |
|---|---|---|
| | | 8); BRRN1(23397); ANKRD36(375248); BCL2L11(10018); IL1F5(26525); |
| | | RIF1(55183); MAP1D(254042); ZAK(51776); FLJ44048(401024); CFLAR |
| | | (8837); CASP8(841); C2orf17(79137); COL4A3(1285); PSMD1(5707); SEPT2 |
| | | (4735); E2F6(1876); TIA1(7072); TEX261(113419); C2orf3(6936); SEPT10 |
| | | (151011); ANAPC1(64682); BIN1(274); ERCC3(2071); MCM6(4175); IFIH1 |
| | | (64135); CLK1(1195); BARD1(580); CUL3(8452); PCAF(8850); PDCD6IP |
| | | (10015); KLHL18(23276); MCM2(4171); ACPP(55); PSMD2(5708); KNG1 |
| | | (3827); OPA1(4976); CIDEC(63924); FYCO1(79443); NME6(10201); RASSF1 |
| | | (11186); NPHP3(27031); ATR(545); KLHL6(89857); HCAP-G(64151); |
| | | HIP2(3093); SEPT11(55752); FGF2(2247); USP38(84640); PRKG2(5593); |
| | | DKK2(27123); NEK1(4750); NIPBL(25836); APC(324); RAD50(10111); |
| | | TCF7(6932); DAP(1611); BIRC1(4671); ATG12(9140); IRF1(3659); NRG2 |
| | | (9542); HDAC3(8841); FGF1(2246); FLJ31951(153830); C6orf153(88745); |
| | | BAG2(9532); FOXO3A(2309); PPP1R14C(81706); DHX16(8449); MDC1(9656); |
| | | CUL7(9820); MCM3(4172); DST(667); DJ122O8.2(57226); SGK(6446); |
| | | PLAGL1(5325); PDCD2(5134); RBAK(57786); UBE2D4(51619); CASP2 |
| | | (835); PMS2(5395); MGC42090(256130); CARD4(10392); STEAP4(79689); |
| | | CDK6(1021); MCM7(4176); FLJ36031(168455); BRAF(673); POLR3D |
| | | (661); PTK2B(2185); FZD3(7976); UNC5D(137970); UBE2V2(7336); SULF1 |
| | | (23213); FZD6(8323); FAM86B1(85002); LZTS1(11178); TNFRSF10A(8797); |
| | | NKX3-1(4824); EXT1(2131); CDC37L1(55664); JAK2(3717); DAPK1 |
| | | (1612); C9orf47(286223); TAF1L(138474); BAG1(573); SEMA4D(10507); TNFSF8 |
| | | (944); FBXW2(26190); MRC1L1(414308); DKK1(22943); SGPL1(8879); |
| | | UNC5B(219699); FAS(355); KIF11(3832); TMEM20(159371); CSPG6 |
| | | (9126); TCF7L2(6934); CASC2(255082); DOCK1(1793); C10orf72(196740); |
| | | C10orf56(219654); SORBS1(10580); FBXW4(6468); FAM26C(255022); |
| | | KIAA1754(85450); DCLRE1A(9937); C10orf46(143384); EIF3S10(8661); |
| | | TIAL1(7073); BNIP3(664); STIM1(6786); WEE1(7465); API5(8536); MDK |
| | | (4192); ATM(472); CD3G(917); EIF4G2(1982); SPTY2D1(144108); FBXO3 |
| | | (26273); FZD4(8322); CTSC(1075); IL18(3606); CACNA1C(775); FLJ20489 |
| | | (55652); TEGT(7009); DAZAP2(9802); PDE1B(5153); CDK2(1017); PA2G4 |
| | | (5036); SOCS2(8835); APAF1(317); GAS2L3(283431); JARID1A(5927); |
| | | NOL1(4839); MCRS1(10445); CDK4(1019); PCTK2(5128); BRCA2(675); |
| | | RB1(5925); FLJ40296(122183); TFDP1(7027); FOXO1A(2308); SAMD4A |
| | | (23034); SLC39A9(55334); GPR65(8477); CIDEB(27141); STRN3(29966); |
| | | SPTLC2(9517); CHES1(1112); FGF7(2252); ARNT2(9915); IGF1R(3480); |
| | | SLC12A6(9990); BMF(90427); PYGO1(26108); PDCD7(10081); LSM16(80153); |
| | | TSC2(7249); DNASE1(1773); UBN1(29855); CYLD(1540); CTCF(10664); |
| | | CDT1(81620); CDK10(8558); AFG3L1(172); GRIN2A(2903); ERN2 |
| | | (10595); APPBP1(8883); SLC12A4(6560); GARNL4(23108); TNFSF12(8742); |
| | | SARM1(23098); NF1(4763); RAB11FIP4(84440); SUZ12(23512); CROP |
| | | (51747); PPM1D(8493); PRKCA(5578); LLGL2(3993); SPHK1(8877); TP53 |
| | | (7157); SPAG5(10615); HDAC5(10014); ANKRD40(91366); TRIM25(7706); |
| | | ERN1(2081); FAM104A(84923); MAPK4(5596); ST8SLA3(51046); MALT1 |
| | | (10892); PHLPP(23239); ESCO1(114799); TXNL4A(10907); DNM2(1785); |
| | | APLP1(333); APOE(348); KLK2(3817); ZNF701(55762); CGB7(94027); |
| | | ZNF415(55786); CEP250(11190); STK4(6789); RIMS4(140730); TP53RK |
| | | (112858); CABLES2(81928); DIDO1(11083); HUNK(30811); BTG3(10950); |
| | | APP(351); SNF1LK(150094); KREMEN1(83999); MCM5(4174); RP5-1104E15.5 |
| | | (54471); EP300(2033); ZC3H7B(23264); PPARA(5465); SF3A1(10291); |
| | | CARD10(29775); SMC1L2(27127); HDAC6(10013); TAF1(6872); ATP7A |
| | | (538); NGFRAP1(27018); RP6-213H9.1(51765); DKC1(1736); ACE2 |
| | | (59272); CXorf38(159013); BTK(695); CUL4B(8450); MOSPD1(56180); |
| | | DAZ2(57055); |
| hsa-miR-368 | 75; | CDKN2C(1031); SH3GLB1(51100); CDC14A(8556); MAN1A2(10905); S100A7L1 |
| | | (338324); MDM4(4194); CIAS1(114548); C1orf135(79000); RIMS3 |
| | | (9783); MAPRE3(22924); LOC165186(165186); SPTBN1(6711); CCT7(10574); |
| | | ZAK(51776); ALS2CR19(117583); COL4A3(1285); PSMD1(5707); |
| | | CREG2(200407); TGFBRAP1(9392); EDAR(10913); ANAPC1(64682); WDR33 |
| | | (55339); TLK1(9874); CALCRL(10203); SATB2(23314); FZD5(7855); |
| | | VHL(7428); GALNTL2(117248); RBM15B(29890); SLC15A2(6565); ACPP |
| | | (55); FAIM(55179); SENP2(59343); ATR(545); SIAH2(6478); TNFSF10 |
| | | (8743); HD(3064); RBPSUH(3516); NFKB1(4790); CCNI(10983); SNCA(6622); |
| | | UNC5C(8633); SKP2(6502); CARD6(84674); APC(324); CSNK1G3(1456); |
| | | UNC5A(90249); PRKAA1(5562); FGF10(2255); ZBED3(84327); ATG1 |

Table 4-45

| | | |
|---|---|---|
| | | 2(9140); IRF1(3659); PPP2CA(5515); E2F3(1871); ZNF192(7745); MAPK13 |
| | | (5603); PARC(23113); PTP4A1(7803); HECA(51696); DAXX(1616); CUL7 |
| | | (9820); SUPT3H(8464); PKHD1(5314); DST(667); CDC2L6(23097); BCLAF1 |
| | | (9774); SERAC1(84947); HDAC9(9734); SEPT7(989); CD36(948); STAG3 |
| | | (10734); RINT1(60561); PMS2(5395); SOSTDC1(25928); SNX13(23161); |
| | | CALN1(83698); CDK5(1020); UNC5D(137970); BAG4(9530); TNFRSF10B |
| | | (8795); CYP7A1(1581); MTSS1(9788); ANKRD15(23189); UNC13B |
| | | (10497); TGFBR1(7046); NFIB(4781); PTPLAD2(401494); FANCG(2189); |
| | | TPM2(7169); CTNNAL1(8727); TNFSF8(944); TRAF1(7185); TSC1(7248); |
| | | SGPL1(8879); UNC5B(219699); FAS(355); BTRC(8945); SUFU(51684); |
| | | IL2RA(3559); C10orf111(221060); MBL2(4153); FBXW4(6468); TIAL1 |
| | | (7073); MKI67(4288); ATM(472); EIF4G2(1982); BRMS1(25855); FGF4(2249); |
| | | FGF3(2248); CASP4(837); TEGT(7009); ESPL1(9700); PA2G4(5036); |
| | | GAS2L3(283431); FZD10(11211); TNFRSF1A(7132); BCAT1(586); CHFR |
| | | (55743); FGF9(2254); CDK8(1024); XPO4(64328); MIPOL1(145282); GPR65 |
| | | (8477); CIDEB(27141); CDKL1(8814); NIN(51199); NEK9(91754); PPP1R13B |
| | | (23368); FGF7(2252); IL16(3603); SCAMP2(10066); DNASE1(1773); |
| | | ELMO3(79767); PKMYT1(9088); ERN2(10595); SEPT1(1731); DNAJA2 |
| | | (10294); FTS(64400); SLC12A4(6560); MPHOSPH6(10200); GSG2(83903); |
| | | ALOX12(239); NLK(51701); NF1(4763); RAPGEFL1(51195); CROP(51747); |
| | | NME2(4831); TLK2(11011); PRKCA(5578); P2RX1(5023); GAS7(8522); |
| | | SSH2(85464); GAS2L2(246176); TBC1D3C(414060); MPP2(4355); HDAC5 |
| | | (10014); PHB(5245); SEPT4(5414); FAM33A(348235); ERN1(2081); SMURF2 |
| | | (64750); CABLES1(91768); MAPRE2(10982); DCC(1630); MALT1 |
| | | (10892); PHLPP(23239); ZNF439(90594); AXL(558); KLK3(354); FLJ38288 |
| | | (284309); MGC71805(403113); PRDX2(7001); ZFP260(339324); SIRT2 |
| | | (22933); C20orf118(140711); CSE1L(1434); BCL2L1(598); PTPRT(11122); |
| | | PTGIS(5740); SON(6651); KREMEN1(83999); NF2(4771); MTP18(51537); |
| | | APOL6(80830); SEPT3(55964); MAPK1(5594); DMC1(11144); CHKB(1120); |
| | | XK(7504); AGTR2(186); WDR42B(139425); ARHGEF9(23229); CUL4B |
| | | (8450); BCAP31(10134); |
| hsa-miR-371 | 76; | TP73(7161); DFFB(1677); H6PD(9563); PAX7(5081); NUDC(10726); UTP11L |
| | | (51118); CDKN2C(1031); PEA15(8682); IL24(11009); CIAS1(114548); |
| | | CTNNBIP1(56998); FRAP1(2475); RCC2(55920); E2F2(1870); CLSPN(63967); |
| | | NOTCH2(4853); TCF7L1(83439); PDCL3(79031); PROC(5624); SEPT2 |
| | | (4735); RTN4(57142); IL1B(3553)BIN1(274); MCM6(4175); FRZB(2487); |
| | | NCKAP1(10787); FZD5(7855); PAX3(5077); FANCD2(2177); VHL(7428); |
| | | PCAF(8850); GNL3(26354); APPL(26060); PSMD2(5708); SENP2(59343); |
| | | TADA3L(10474); WHSC1(7468); HD(3064); FGF2(2247); DKK2(27123); |
| | | UNC5A(90249); GDNF(2668); CDC23(8697); NRG2(9542); CD14(929); |
| | | CSNK1A1(1452); RIPK1(8737); E2F3(1871); GMNN(51053); TCF19(6941); |
| | | MAPK13(5603); CDRN1A(1026); PARC(23113); FOXO3A(2309); TNFAIP3 |
| | | (7128); DAXX(1616); FRK(2444); MAP3K5(4217); PLAGL1(5325); |
| | | AHR(196); FZD9(8326); MAD1L1(8379); CYCS(54205); CDK6(1021); MCM7 |
| | | (4176); E2F5(1875); LZTS1(11178); BIN3(55909); ADRA1A(148); DUSP4 |
| | | (1846); JAK2(3717); RECK(8434); GADD45G(10912); INVS(27130); CDK9 |
| | | (1025); ABL1(25); FLJ20245(54863); CDKN2B(1030); BAG1(573); SEMA4D |
| | | (10507); PTCH(5727); CARD9(64170); ANAPC2(29882); SIRT1(23411); |
| | | SGPL1(8879); PTEN(5728); NOLC1(9221); PDCD11(22984); BAG3 |
| | | (9531); PARD3(56288); FZD8(8325); SPOCK2(9806); DLG5(9231); SFRP5 |
| | | (6425); C10orf46(143384); API5(8539); INCENP(3619); DPF2(5977); LRP5 |
| | | (4041); INPPL1(3636); CD3E(916); LOH11CR2A(4013); VPS26B(112936); |
| | | IGF2(3481); WT1(7490); BAD(572); BRMS1(25855); FGF3(2248); PAK1 |
| | | (5058); FZD4(8322); SESN3(143686); BCL9L(283149); H2AFX(3014); CDK2 |
| | | (1017); CRADD(8738); KNTC1(9735); FZD10(11211); KRAS(3345); C12orf22 |
| | | (81566); PAWR(5074); ANAPC7(51434); HRK(8739); ANAPC5(51433); |
| | | DIABLO(56616); CDK2AP1(8099); BRCA2(675); SUGT1(10910); ING1 |
| | | (3621); LOC440157(440157); CDKN3(1033); PSEN1(5663); C14orf132 |
| | | (56967); TRAF3(7187); GZMH(2999); STRN3(29966); ZFYVE1(53349); MOAP1 |
| | | (64112); AKT1(207); JAG2(3714); IGF1R(3480); FLJ22795(80154); |
| | | FLJ36208(283948); CCNF(899); RBL2(5934); CTCF(10664); CLCN7(1186); |
| | | PKMYT1(9088); ERN2(10595); PYCARD(29108); CSNK2A2(1459); TRADD |
| | | (8717); SMPD3(55512); FAM38A(9780); HIC1(3090); ALOX12(239); |
| | | TNFSF12(8742); MAPK7(5598); FZD2(2535); MAPT(4137); NGFR(4804); |
| | | PPM1D(8493); TLK2(11011); SPHK1(8877); BIRC5(332); FLJ44861(388 |

Table 4-46

| | | |
|---|---|---|
| | | 428); ANAPC11(51529); GPS1(2873); MNT(4335); TP53(7157); BECN1(8678); |
| | | ERN1(2081); CBX4(8535); MAPRE2(10982); STK11(6794); DAZAP1 |
| | | (26528); FZR1(51343)HMG20B(10362); PIN1(5300); F2RL3(9002); SSBP4 |
| | | (170463); PSMD8(5714); MAP3K10(4294); ARHGEF1(9138); AES(166); |
| | | CDC37(11140); PPP1R13L(10848); BBC3(27113); LIG1(3978); CBFA2T2 |
| | | (9139); CSE1L(1434); SS18L1(26039); PCNA(5111); FLJ41733(400870); |
| | | BTG3(10950); APP(351); SNF1LK(150094); GAS2L1(10634); PGEA1(25776); |
| | | ARVCF(421); MN1(4330); SSTR3(6753); NHP2L1(4809); WNT7B(7477); |
| | | PCTK1(5127); DKC1(1736); SEPT6(23157); HCFC1(3054); MTCP1(4515); |
| hsa-miR-376a | 77; 78; 757; | MACF1(23499); PRKACB(5567); SH3GLB1(51100); CDC14A(8556); BCL9 |
| hsa-miR-376b | 2251; | (607); CKS1B(1163); MDM4(4194); KIAA1804(84451); CCNL2(81669); FRAP1 |
| | | (2475); CASP9(842); HSPB7(27129); E2F2(1870); C1orf135(79000); |
| | | RHBDL2(54933); KIAA0494(9813); TMEM48(55706); NOTCH2(4853); FH |
| | | (2271); PPP1CB(5500); PRKCE(5581); CCT7(10574); KCNIP3(30818); |
| | | BRRN1(23397); BCL2L11(10018); RIF1(55183); G6PC2(57818); ZAK(51776); |
| | | PMS1(5378); COQ10B(80219); PLCL1(5334); BMPR2(659); CREB1(1385); |
| | | PTMA(5757); CARD12(58484); TGFA(7039); TGFBRAP1(9392); ANAPC1 |
| | | (64682); ERCC3(2071); WDR33(55339); MCM6(4175); TLK1(9874); |
| | | CERKL(375298); STAT1(6772); STK17B(9262); PGAP1(80055); BARD1 |
| | | (580); PAX3(5077); CUL3(8452); TRNT1(51095); GALNTL2(117248); PCAF |
| | | (8850); CCR2(1231); CD200(4345); NEK11(79858); RAP2B(5912); PSMD2 |
| | | (5708); PAK2(5062); ANKRD28(23243); THRB(7068); GORASP1(64689); |
| | | NME6(10201); RASSF1(11186); NEK4(6787); DCP1A(55802); DNASE1L3 |
| | | (1776); FHIT(2272); ZNF80(7634); ATR(545); LIPH(200879); SLC34A2 |
| | | (10568); FGF5(2250); NFKB1(4790); PLK4(10733); PHF17(79960); GAB1 |
| | | (2549); TLR2(7097); CCNI(10983); TIFA(92610); FLJ23191(79625); SLC7A11 |
| | | (23657); SKP2(6502); NIPBL(25836); CARD6(84674); ERBB2IP(55914); |
| | | APC(324); CSNK1G3(1456); SGCD(6444); UNC5A(90249); SEMA5A |
| | | (9037); PRLR(5618); HCN1(348380); ELOVL7(79993); P18SRP(285672); TICAM2 |
| | | (353376); ATG12(9140); PPP2CA(5515); HNRPA0(10949); NME5 |
| | | (8382); HDAC3(8841); E2F3(1871); GMNN(51053); CSNK2B(1460); MAPK13 |
| | | (5603); PARC(23113); QKI(9444); NEDD9(4739); IHPK3(117283); DST |
| | | (667); CCNC(892); CDC2L6(23097); FRK(2444); LATS1(9113); LPAL2(80350); |
| | | AHR(196); HDAC9(9734); SEPT7(989); WBSCR19(285955); LOC441257 |
| | | (441257); STAG3(10734); MGC57359(441272); PAPOLB(56903); TRIAD3 |
| | | (54476); PMS2(5395); SNX13(23161); CYCS(54205); CARD4(10392); |
| | | ELMO1(9844); SFRP4(6424); CDK5(1020); SLC7A2(6542); PTK2B(2185); |
| | | FZD3(7976); UNC5D(137970); BAG4(9530); SULF1(23213); TERF1 |
| | | (7013); PI15(51050); POP1(10940); KCNS2(3788); FZD6(8323); TRIM35(23087); |
| | | PLAG1(5324); STK3(6788); EIF2C2(27161); JAK2(3717); DAPK1 |
| | | (1612); ABL1(25); SEMA4D(10507); TNFSF8(944); TSC1(7248); ZMYND11 |
| | | (10771); CDC2(983); UNC5B(219699); FAS(355); INA(9118); ACSL5(51703); |
| | | BAG3(9531); DOCK1(1793); C10orf111(221060); CUL2(8453); PPP3CB |
| | | (5532); DLG5(9231); TIAL1(7073); MKI67(4288); TRIM22(10346); GAS2 |
| | | (2620); LUZP2(338645); TMEM16C(63982); ZFP91(80829); ATM(472); EIF4G2 |
| | | (1982); RELA(5970); BRMS1(25855); FCHSD2(9873); RAB30(27314); |
| | | MTMR2(8898); LTBR(4055); PTPN6(5777); PA2G4(5036); HMGA2(8091); |
| | | NAV3(89795); APAF1(317); GAS2L3(283431); CUTL2(23316); FBXW8 |
| | | (26259); KNTC1(9735); TMEM132B(114795); PARP11(57097); HDAC7A |
| | | (51564); DDIT3(1649); WIF1(11197); CHFR(55743); FGF9(2254); CDK8 |
| | | (1024); RFP2(10206); SUGT1(10910); GPC6(10082); CDC16(8881); STYX |
| | | (6815); PSEN1(5663); CIDEB(27141); CDKL1(8814); WDR22(8816); PPP1R13B |
| | | (23368); KIF23(9493); CSK(1445); FBXO22(26263); IL16(3603); SCAND2 |
| | | (54581); IGF1R(3480); DAPK2(23604); TSC2(7249); KREMEN2 |
| | | (79412); DNASE1(1773); KATNB1(10300); ELMO3(79767); NIP7(51388); |
| | | GR1N2A(2903); BOLA2(552900); SEPT1(1731); SLC12A4(6560); NF1(4763); |
| | | TADA2L(6871); CROP(51747); NME1(4830); NME2(4831); MAP2K6 |
| | | (5608); P2RX1(5023); SPAG5(10615); NLE1(54475); TBC1D3C(414060); SEPT4 |
| | | (5414); HELZ(9931); MAPRE2(10982); MALT1(10892); DSC3(1825); |
| | | BRUNOL4(56853); TXNL1(9352); MBP(4155); ZNF439(90594); MGC26694 |
| | | (284439); ZNF254(9534); AXL(558); ZNF230(7773); PPP2R1A(5518); |
| | | ZBTB7A(51341); MGC71805(403113); SIRT2(22933); TGFB1(7040); ZNF180 |
| | | (7733); ERCC2(2068); RAB22A(57403); BIRC7(79444); E2F1(1869); GGTL3 |
| | | (2686); RBL1(5933); PTPRT(11122); SON(6651); TRPM2(7226); C21 |

Table 4-47

| | | |
|---|---|---|
| | | orf66(94104); BRWD1(54014); LOC400924(400924); MCM5(4174); SEPT3 |
| | | (55964); SMC1L2(27127); PCTK1(5127); HDAC6(10013); MAGEH1(28986); |
| | | MLLT7(4303); STAG2(10735); RP6-213H19.1(51765); FIGF(2277); RAB9B |
| | | (51209); RP13-383K5.1(55285); LAMP2(3920); DAZ2(57055); |
| hsa-miR-377 | 79; 758; 759; | DFFB(1677); TNFRSF1B(7133); NUDC(10726); SESN2(83667); RCC1(1104); |
| | | LCK(3932); RBBP4(5928); MACF1(23499); CDC20(991); FNBP1L(54874); |
| | | CDC14A(8556); RAP1A(5906); BCL9(607); RGS2(5997); G0S2(50486); |
| | | CIAS1(114548); TNFRSF14(8764); TNFRSF9(3604); CTNNBIP1(56998); |
| | | FRAP1(2475); ZBTB17(7709); FOXJ3(22887); TAL1(6886); LRP8(7804); |
| | | BCL10(8915); SORT1(6272); NOTCH2(4853); PYGO2(90780); CD5L |
| | | (922); C1orf147(574431); BIRC6(57448); PRKCE(5581); MSH2(4436); CCT7 |
| | | (10574); HK2(3099); BCL2L11(10018); CCNT2(905); RIF1(55183); TAIP-2 |
| | | (80034); GLS(2744); SLC39A10(57181); ABI2(10152); CD28(940); NRP2 |
| | | (8828); PTMA(5757); SEPT2(4735); BOK(666); E2F6(1876); CGREF1(10669); |
| | | CARD12(58484); LRPPRC(10128); TIA1(7072); BUB1(699); ANAPC1 |
| | | (64682); STK17B(9262); CLK1(1195); ERBB4(2066); BARD1(580); TNS1 |
| | | (7145); PAX3(5077); CUL3(8452); PCAF(8850); MLH1(4292); ZNF621 |
| | | (285268); APPL(26060); NEK11(79858); ARMC8(25852); USP13(8975); PSMD2 |
| | | (5708); TP73L(8626); OPA1(4976); TADA3L(10474); RAF1(5894); ZCSL2 |
| | | (285381); RASSF1(11186); TUSC4(10641); UBE1C(9039); RAB6B(51560) |
| | | PIK3CB(5291); TFDP2(7029); WHSC1(7468); MRFAP1(93621); CD38 |
| | | (952); ANAPC4(29945); IL8(3576); SEPT11(55752); CCNG2(901); FRAS1 |
| | | (80144); NFKB1(4790); TLR2(7097); STOX2(56977); GAK(2580); LRPAP1 |
| | | (4043); GABRG1(2565); PRKG2(5593); LEF1(51176); CASP6(839); FAM105B |
| | | (90268); NIPBL(25836); APC(324); CSNK1G3(1456); FLJ37562(134553); |
| | | G3BP(10146); PRLR(5618); SEMA6A(57556); IRF1(3659); PPP2CA |
| | | (5515); KLHL3(26249); CDC25C(995); PFDN1(5201); CSNK1A1(1452); |
| | | DUSP1(1843); IBRDC2(255488); E2F3(1871); TCF19(6941); MAPK13(5603); |
| | | PARC(23113); VEGF(7422); PNRC1(10957); LIN28B(389421); C6orf71 |
| | | (389434); QKI(9444); IER3(8870); CCND3(896); DST(667); SESN1(27244); |
| | | SGK(6446); BCLAF1(9774); PLAGL1(5325); LATS1(9113); PDCD2(5134); |
| | | WIPI2(26100); HDAC9(9734); SEPT7(989); STK17A(9263); LOC441257 |
| | | (441257); STAG3(10734); BCAP29(55973); NRF1(4899); MKLN1(4289); |
| | | CASP2(835); CUL1(8454); RBM33(155435); PMS2(5395); CYCS(54205); |
| | | ELMO1(9844); INHBA(3624); PURB(5814); YWHAG(7532); CDK6(1021); |
| | | UBE2H(7328); SLC35B4(84912); TNKS(8658); POLR3D(661); PTK2B |
| | | (2185); TACC1(6867); FZD6(8323); TRIM35(23087); RAB11FIP1(80223); |
| | | DKK4(27121); TNFRSF11B(4982); FBXO32(114907); JRK(8629); ANKRD15 |
| | | (23189); JAK2(3717); UNC13B(10497); RECK(8434); DAPK1(1612); |
| | | C9orf28(89853); GPR107(57720); ABL1(25); IFNA2(3440); CDKN2B(1030); |
| | | BAG1(573); WDR40A(25853); RNF38(152006); IGFBPL1(347252); SEMA4D |
| | | (10507); SPTLC1(10558); PTCH(5727); TNFSF8(944); FAS(355); C10orf132 |
| | | (401647); BTRC(8945); PDCD11(22984); TCF7L2(6934); C10orf72 |
| | | (196740); PPP3CB(5532); DLG5(9231); SMNDC1(10285); C10orf46(143384); |
| | | STIM1(6786); WEE1(7465); HIPK3(10114); EXT2(2132); SIPA1(6494); |
| | | LRP5(4041); FADD(8772); BIRC2(329); ATM(472); CD3E(916); LOH11CR2A |
| | | (4013); DKK3(27122); WT1(7490); HRASLS3(11145); RELA(5970); |
| | | PPP1CA(5499); PAK1(5058); FZD4(8322); PPP2R1B(5519); BCL9L(283149); |
| | | CDON(50937); ETS1(2113); CCND2(894); TNFRSF7(939); CDKN1B |
| | | (1027); PA2G4(5036); MDM2(4193); CLLU1(574028); SOCS2(8835); APAF1 |
| | | (317); KNTC1(9735); TMEM132B(114795); FZD10(11211); RAN(5901); |
| | | KRAS(3845); MGC24039(160518); HDAC7A(51564); WIF1(11197); DUSP6 |
| | | (1848); MMAB(326625); GOLGA3(2802); CHFR(55743); TNFRSF19 |
| | | (55504); BRCA2(675); RB1(5925); CUL4A(8451); LATS2(26524); KATNAL1 |
| | | (84056); NEK3(4752); KIAA1008(22894); ZIC5(85416); LIG4(3981); BCL2L2 |
| | | (599); PNN(5411); TXNDC(81542); CGRRF1(10668); PSEN1(5663); VASH1 |
| | | (22846); LTBP2(4053); NEK9(91754); CHES1(1112); BAG5(9529); JAG2 |
| | | (3714); KLF13(51621); SNX1(6642); KIF23(9493); IGF1R(3480); BMF |
| | | (90427); BCL2L10(10017); CSNK1G1(53944); PDCD7(10081); TSC2(7249); |
| | | CCNF(899); PRO0149(29035); ITGAM(3684); CARD15(64127); RBL2 |
| | | (5934); ELMO3(79767); CTCF(10664); CDK10(8558); AXIN1(8312); GSPT1 |
| | | (2935); USP31(57478); LOC440350(440350); SEPT1(1731); FTS(64400); |
| | | CIAPIN1(57019); SLC12A4(6560); MPHOSPH6(10200); Pfs2(51659); PAFAH1B1 |
| | | (5048); NTN1(9423); MAPK7(5598); LIG3(3980); MLLT6(4302); |
| | | LASP1(3927); CDC6(990); MAPT(4137); NGFR(4804); PPM1D(8493); TL |

Table 4-48

| | | |
|---|---|---|
| | | K2(11011); PRKCA(5578); BIRC5(332); FOXK2(3607); GAS7(8522); NUFIP2 |
| | | (57532); BRCA1(672); PHB(5245); SFRS1(6426); SEPT4(5414); ERN1 |
| | | (2081); CABLES1(91768); SETBP1(26040); SMAD4(4089); MALT1(10892); |
| | | ESCO1(114799); BCL2(596); TNFSF9(8744); FLJ21742(84167); CCNE1 |
| | | (898); MAP3K10(4294); ZNF230(7773); FOSB(2354); PPP2R1A(5518); NALP2 |
| | | (55655); CDC37(11140); ZNF529(57711); LIG1(3978); KLK10(5655); |
| | | MCM8(84515); PLCB1(23236); CSE1L(1434); CSNK2A1(1457); BCL2L1 |
| | | (598); E2F1(1869); NCOA6(23054); GGTL3(2686); RBL1(5933); ATP9A(10079); |
| | | SYCP2(10388); CABLES2(81928); USP16(10600); SON(6651); BTG3 |
| | | (10950); ADAMTS5(11096); WDR4(10785); SNF1LK(150094); CDC45L |
| | | (8318); MCM5(4174); EP300(2033); SEPT3(55964); BIK(638); GTSE1(51512); |
| | | MAPK1(5594); PPM1F(9647); CHEK2(11200); SSTR3(6753); NHP2L1 |
| | | (4809); CCDC22(28952); MAGEH1(28986); dJ341D10.1(286453); PRPS1 |
| | | (5631); BIRC4(331); STAG2(10735); CXorf6(10046); IKBKG(8517); FIGF |
| | | (2277); SMC1L1(8243); CHM(1121); HCFC1(3054); |
| hsa-miR-378 | 80; 2252; 2253; | NUDC(10726); SESN2(83667); KHDRBS1(10657); RBBP4(5928); MACF1 |
| | | (23499); CDKN2C(1031); SH3GLB1(51100); CD2(914); CACNA1E(777); |
| | | IL24(11009); CIAS1(114548); TNFRSF14(8764); HES2(54626); DFFA(1676); |
| | | FRAP1(2475); CLSPN(63967); FAF1(11124); SASS6(163786); NOTCH2 |
| | | (4853); ENSA(2029); RP1-14N1.3(388698); PYGO2(90780); ISG20L2 |
| | | (81875); RIPK5(25778); IL10(3586); TP53BP2(7159); ENAH(55740); MAPRE3 |
| | | (22924); BIRC6(57448); CCT7(10574); HTRA2(27429); BRRN1(23397); |
| | | RIF1(55183); ZAK(51776); CFLAR(8837); CASP10(843); CD28(940); ALS2CR19 |
| | | (117583); PSMD1(5707); ANAPC1(64682); ERCC3(2071); GPR155 |
| | | (151556); FRZB(2487); HDAC4(9759); FANCD2(2177); VHL(7428); ZNF620 |
| | | (253639); MCM2(4171); FAIM(55179); OPA1(4976); NME6(10201); |
| | | GSK3B(2932); MGC40579(256356); ATR(545); SIAH2(6478); HD(3064); SEPT11 |
| | | (55752); CCNG2(901); FGF5(2250); SPP1(6696); FGF2(2247); LETM1 |
| | | (3954); LRPAP1(4043); APBB2(323); ASAHL(27163); CASP3(836); NIPBL |
| | | (25836); ERBB2IP(55914); F2R(2149); APC(324); TCF7(6932); LSM11 |
| | | (134353); FGF10(2255); BIRC1(4671); MCC(4163); IRF1(3659); CDC25C |
| | | (995); DNAJC18(202052); CD14(929); CSNK1A1(1452); E2F3(1871); MAPK13 |
| | | (5603); PARC(23113); KIAA1919(91749); HECA(51696); SASH1(23328); |
| | | MDC1(9656); DAXX(1616); NUDT3(11165); CCND3(896); CUL7(9820); |
| | | DST(667); SESN1(27244); MAP3K5(4217); PDCD2(5134); SEPT7(989); |
| | | EGFR(1956); GATAD1(57798); ZKSCAN1(7586); RINT1(60561); BCAP29 |
| | | (55973); SNX8(29886); TRIAD3(54476); CYCS(54205); CARD4(10392); |
| | | HIP1(3092); HIPK2(28996); PTK2B(2185); ESCO2(157570); UNC5D |
| | | (137970); UBE2V2(7336); TERF1(7013); CTSB(1508); LZTS1(11178); |
| | | TNFRSF10D(8793); ADRA1A(148); TRIM35(23087); RAB11FIP1(80223); WDR21C |
| | | (138009); ANKRD15(23189); OSTF1(26578); C9orf47(286223); INVS |
| | | (27130); BAG1(573); PTCH(5727); TNFSF15(9966); TNFSF8(944); TRAF1 |
| | | (7185); DKK1(22943); CDC2(983); SGPL1(8879); LDB3(11155); BTRC |
| | | (8945); SUFU(51684); PDCD11(22984); BAG3(9531); IL2RA(3559); ITIH5 |
| | | (80760); PARD3(56288); C10orf46(143384); MKI67(4288); TUB(7275); API5 |
| | | (8539); EXT2(2132); DPF2(5977); SSSCA1(10534); LRP5(4041); ATM(472); |
| | | CCDC15(80071); CHEK1(1111); IGF2(3481); HRASLS3(11145); MAP3K11 |
| | | (4296); RELA(5970); SPTBN2(6712); CASP4(837); PPP2R1B(5519); BCL9L |
| | | (283149); CCND2(894); CNAP1(9918); ETV6(2120); IAPP(3375); CRADD |
| | | (8738); KNTC1(9735); LRP6(4040); MCRS1(10445); FAIM2(23017); |
| | | C12orf22(81566); CDK4(1019); BTG1(694); ANAPC7(51434); HSMPP8 |
| | | (54737); TNFRSF19(55504); BRCA2(675); RFP2(10206); ING1(3621); LATS2 |
| | | (26524); CSNK1A1L(122011); C14orf111(51077); VASH1(22846); ACIN1 |
| | | (22985); CIDEB(27141); CDKL1(8814); LTBP2(4053); TGFB3(7043); CHES1 |
| | | (1112); PPP1R13B(23368); JAG2(3714); IVD(3712); MAPK6(5597); |
| | | BMF(90427); PDCD7(10081); C15orf38(348110); DNAJA3(9093); SPN(6693); |
| | | CARD15(64127); PARD6A(50855); C16orf28(65259); FTS(64400); HIC1 |
| | | (3090); NF1(4763); RHBDL3(162494); CDC6(990); MAPT(4137); PRKCA |
| | | (5578); SPHK1(8877); GPS1(2873); SMYD4(114826); NALP1(22861); |
| | | TP53(7157); SLC25A35(399512); GAS7(8522); TOM1L2(146691); SPAG5 |
| | | (10615); GAS2L2(246176); BRCA1(672); HDAC5(10014); GJA7(10052); |
| | | CSNK1D(1453); MAPRE2(10982); CHAF1A(10036); TNFAIP8L1(126282); |
| | | UHRF1(29128); GATAD2A(54815); ZNF431(170959); APOE(348); FLJ45684 |
| | | (400666); TNFSF14(8740); DNASE2(1777); HM13(81502); STK4(6789); |
| | | UBE2C(11065); BIRC7(79444); E2F1(1869); RBL1(5933); CTCFL(14 |

Table 4-49

| | | |
|---|---|---|
| | | 0690); SYCP2(10388); DIDO1(11083); CHRNA4(1137); SON(6651); MX1 |
| | | (4599); SNF1LK(150094); ITGB2(3689); SMARCB1(6598); KREMEN1(83999); |
| | | EP300(2033); SEPT3(55964); PPARA(5465); DMC1(11144); HDAC6 |
| | | (10013); TAF1(6872); BIRC4(331); STAG2(10735); IKBKG(8517); SMC1L1 |
| | | (8243); BTK(695); HCFC1(3054); MTCP1(4515); |
| hsa-miR-380-5p | 81; 760; 916; | TP73(7161); TESK2(10420); LPGAT1(9926); ITPKB(3707); BIRC6(57448); |
| | | GKN1(56287); CD28(940); BIN1(274); ERCC3(2071); FRZB(2487); PAX3 |
| | | (5077); PDCD6IP(10015); KNG1(3827); OPA1(4976); ATR(545); KLHL6 |
| | | (89857); HD(3064); ADAM29(11086); MRPS30(10884); CSNK1G3(1456); RAD50 |
| | | (10111); MCC(4163); ATG12(9140); SEMA6A(57556); CDC25C(995); |
| | | TNF(7124); IL17(3605); PTP4A1(7803); SASH1(23328); TNFRSF21(27242); |
| | | FRK(2444); LATS1(9113); CUL1(8454); TRIAD3(54476); CARD4(10392); |
| | | ELMO1(9844); HIPK2(28996); UNC5D(137970); SLC30A8(169026); |
| | | ADRA1A(148); PLAG1(5324); JAK2(3717); DAPK1(1612); NCBP1(4686); |
| | | ABL1(25); CDKN2B(1030); PTCH(5727); TNFSF8(944); C10orf9(219771); |
| | | BTRC(8945); TCF7L2(6934); DOCK1(1793); CXCL12(6387); DLG5(9231); |
| | | EIF4G2(1982); FZD4(8322); TEGT(7009); RBMS2(5939); ANKRD13(88455); |
| | | KNTC1(9735); TNFRSF19(55504); ATP8A2(51761); BRCA2(675); CUL4A |
| | | (8451); TFDP1(7027); FOXO1A(2308); DZIP1(22873); VASH1(22846); |
| | | TRAF3(7187); EXOC5(10640); JAG2(3714); RNF111(54778); UBE3A(7337); |
| | | KATNB1(10300); AXIN1(8312); GSPT1(2935); MAPK7(5598); NLK |
| | | (51701); MAPT(4137); TLK2(11011); PRKCA(5578); CARD14(79092); MPO |
| | | (4353); TMC6(11322); CETN1(1068); BCL2(596); UHRF1(29128); TNFSF9 |
| | | (8744); ARHGEF1(9138); BCL3(602); FOSB(2354); NALP2(55655); TNFSF14 |
| | | (8740); ERCC2(2068); TRIB3(57761); C20orf91(284800); USP16(10600); |
| | | KREMEN1(83999); MTP18(51537); EP300(2033); CARD10(29775); |
| | | PCTK1(5127); CCNB3(85417); CUL4B(8450); |
| hsa-miR-382 | 82; 761; 762; | PRKCZ(5590); TP73(7161); PAX7(5081); RCC1(1104); RBBP4(5928); CKS1B |
| | | (1163); DAP3(7818); IL24(11009); TRAF5(7188); TGFB2(7042); SASS6 |
| | | (163786); NRAS(4893); NOTCH2(4853); CD5L(922); ASPM(259266); IL10 |
| | | (3586); ENAH(55740); MAPRE3(22924); MSH2(4436); CCT7(10574); HTRA2 |
| | | (27429); BCL2L11(10018); RIF1(55183); ZAK(51776); PMS1(5378); BMPR2 |
| | | (659); SEPT10(151011); BUB1(699); IL1A(3552); STAT1(6772); PAX3 |
| | | (5077); CUL3(8452); DTYMK(1841); PDCD6IP(10015); NEK11(79858); |
| | | SMC4L1(10051); FXR1(8087); PSMD2(5708); OPA1(4976); RAF1(5894); |
| | | NEK4(6787); ATR(545); SEPT11(55752); FGF5(2250); FGF2(2247); PLK4 |
| | | (10733); UGT2B15(7366); CCNI(10983); NEK1(4750); CASP3(836); NIPBL |
| | | (25836); CCNB1(891); RAD17(5884); F2R(2149); APC(324); BNIP1(662); |
| | | PRLR(5618); SEMA6A(57556); IRF1(3659); FBXW11(23291); RIPK1(8737); |
| | | GMNN(51053); BRPF3(27154); PARC(23113); PTP4A1(7803); TTK(7272); |
| | | MDC1(9656); DAXX(1616); CCND3(896); DST(667); SGK(6446); LATS1 |
| | | (9113); PDCD2(5134); AHR(196); HDAC9(9734); RINT1(60561); BCAP29 |
| | | (55973); MAD1L1(8379); TRIAD3(54476); SOSTDC1(25928); CARD4 |
| | | (10392); INHBA(3624); CDK6(1021); BRAF(673); AGPAT5(55326); UNC5D |
| | | (137970); BAG4(9530); E2F5(1875); SLC26A7(115111); FZD6(8323); TNFRSF10A |
| | | (8797); TRIM35(23087); DUSP4(1846); SFRP1(6422); TP53INP1 |
| | | (94241); STK3(6788); TNFRSF11B(4982); ANKRD15(23189); UNC13B |
| | | (10497); IFNA2(3440); CDKN2B(1030); SEMA4D(10507); PPP6C(5537); ARL5B |
| | | (221079); SIRT1(23411); SGPL1(8879); SEC24C(9632); PTEN(5728); |
| | | KIF11(3832); NOLC1(9221); PDCD11(22984); CSPG6(9126); BAG3(9531); |
| | | C10orf63(219670); CUL2(8453); C10orf46(143384); MKI67(4288); HIPK3 |
| | | (10114); API5(8539); CCND1(595); FADD(8772); BIRC3(330); CUL5(8065); |
| | | ATM(472); IGF2(3481); EIF4G2(1982); MTL5(9633); FZD4(8322); SESN3 |
| | | (143686); PPP2R1B(5519); CCND2(894); CNAP1(9918); IAPP(3375); |
| | | APAF1(317); GAS2L3(283431); KNTC1(9735); RAN(5901); PAWR(5074); |
| | | LIN7A(8825); PCTK2(5128); RFP2(10206); CDC16(8881); LIG4(3981); HSPA2 |
| | | (3306); TRAF3(7187); DAD1(1603); EGLN3(112399); ATXN3(4287); |
| | | AKT1(207); MAPK6(5597); CSK(1445); SCAND2(54581); PRTG(283659); |
| | | BNIP2(663); BCL2A1(597); KREMEN2(79412); RBL2(5934); DNAJA2(10294); |
| | | FTS(64400); SLC12A4(6560); TXNL4B(54957); TNFSF12(8742); NLK(51701); |
| | | NF1(4763); LIG3(3980); TADA2L(6871); CROP(51747); CSNK1D |
| | | (1453); SERP1NB2(5055); ROCK1(6093); PARD6G(84552); AXL(558); |
| | | FLJ00060(90011); NALP2(55655); TRIB3(57761); STK4(6789); CSNK2A1 |
| | | (1457); PCNA(5111); APP(351); ADAMTS5(11096); ITGB2(3689); YWHAH |
| | | (7533); EP300(2033); SEPT3(55964); CHEK2(11200); NRK(203447); C |

Table 4-50

| | | |
|---|---|---|
| | | D40LG(959); SMC1L1(8243); CUL4B(8450); |
| hsa-miR-412 | 83; 763; | PRKCZ(5590); PAX7(5081); RCC1(1104); LCK(3932); RBBP4(5928); UTP11L |
| | | (51118); MACF1(23499); CDKN2C(1031); CDC7(8317); BNIPL(149428); |
| | | PEA15(8682); UHMK1(127933); ADORA1(134); MDM4(4194); IL24(11009); |
| | | G0S2(50486); TGFB2(7042); CCNL2(81669); CDC2L2(985); TNFRSF25 |
| | | (8718); FRAP1(2475); CASP9(842); RCC2(55920); CLSPN(63967); TESK2 |
| | | (10420); MKNK1(8569); PYGO2(90780); DEDD(9191); ASPM(259266); |
| | | PLEKHA6(22874); IL10(3586); YOD1(55432); TP53BP2(7159); RHOB |
| | | (388); PPP1CB(5500); BIRC6(57448); PRKCE(5581); MSH2(4436); HK2(3099); |
| | | BCL2L11(10018); RIF1(55183); CASP10(843); CASP8(841); FZD7(8324); |
| | | CD28(940); ALS2CR19(117583); COL4A3(1285); BOK(666); PPM1G |
| | | (5496); CARD12(58484); EIF2AK2(5610); ANAPC1(64682); IFIH1(64135); |
| | | FRZB(2487); CLK1(1195); PAX3(5077); CUL3(8452); HDAC4(9759); FANCD2 |
| | | (2177); OXNAD1(92106); PCAF(8850); PDCD6IP(10015); NKTR(4820); |
| | | RBM5(10181); EAF2(55840); RNF7(9616); SMC4L1(10051); PSMD2 |
| | | (5708); TP73L(8626); TRAIP(10293); NEK4(6787); DNASE1L3(1776); STAG1 |
| | | (10274); ATR(545); HCAP-G(64151); SEPT11(55752); CCNG2(901); CASP6 |
| | | (839); SFRP2(6423); NEK1(4750); CASP3(836); NIPBL(25836); CARD6 |
| | | (84674); MRPS30(10884); ERBB2IP(55914); CCNB1(891); F2R(2149); |
| | | APC(324); GALNT10(55568); BNIP1(662); PRLR(5618); PRKAA1(5562); |
| | | UNG2(10309); BIRC1(4671); MCC(4163); SEMA6A(57556); CDC25C(995); |
| | | NRG2(9542); PFDN1(5201); FBXW11(23291); RIPK1(8737); CSNK2B |
| | | (1460); PARC(23113); VEGF(7422); FOXO3A(2309); HECA(51696); SASH1 |
| | | (23328); NEDD9(4739); BAK1(578); CUL7(9820); TNFRSF21(27242); PKHD1 |
| | | (5314); DST(667); FRK(2444); MAP3K5(4217); LATS1(9113); PDCD2 |
| | | (5134); SEPT7(989); EGFR(1956); STAG3(10734); BCAP29(55973); CASP2 |
| | | (835); MAD1L1(8379); ELMO1(9844); SFRP4(6424); INHBA(3624); |
| | | MCM7(4176); EPHA1(2041); TNKS(8658); TNFRSF10C(8794); PTK2B(2185); |
| | | ESCO2(157570); TACC1(6867); TRIM35(23087); CLU(1191); SFRP1 |
| | | (6422); DKK4(27121); REXO1L1(254958); TP53INP1(94241); STK3(6788); |
| | | ANKRD15(23189); NUDT2(318); UNC13B(10497); CDK9(1025); ABL1(25); |
| | | TAF1L(138474); BAG1(573); SEMA4D(10507); TNFSF8(944); TRAF1 |
| | | (7185); CIZ1(25792); TSC1(7248); CARD9(64170); ANAPC2(29882); PTEN |
| | | (5728); FRAT1(10023); BTRC(8945); NOLC1(9221); PDCD11(22984); TCF7L2 |
| | | (6934); DOCK1(1793); PPP3CB(5532); DLG5(9231); FRAT2(23401); |
| | | SFRP5(6425); DCLRE1A(9937); TIAL1(7073); MKI67(4288); STIM1(6786); |
| | | TUB(7275); WEE1(7465); GAS2(2620); API5(8539); MDK(4192); F2(2147); |
| | | INCENP(3619); ATM(472); EI24(9538); HRAS(3265); EIF4G2(1982); |
| | | CLCF1(23529); FZD4(8322); CASP1(834); BCL9L(283149); CCND2(894); |
| | | CNAP1(9918); PTPN6(5777); DDX11(1663); ESPL1(9700); PDE1B(5153); |
| | | MDM2(4193); CCT2(10576); KNTC1(9735); FZD10(11211); RAN(5901); |
| | | NOL1(4839); KRAS(3845); C12orf22(81566); PPP1CC(5501); SCARB1(949); |
| | | CHFR(55743); FGF9(2254); CDK8(1024); LATS2(26524); LIG4(3981); |
| | | PSEN1(5663); VASH1(22846); GZMH(2999); GZMB(3002); STRN3(29966); |
| | | NFKBIA(4792); C14orf43(91748); LTBP2(4053); NEK9(91754); TGFB3 |
| | | (7043); CHES1(1112); PPP1R13B(23368); AKT1(207); BUB1B(701); CCNB2 |
| | | (9133); SMAD3(4088); SLC12A6(9990); DAPK2(23604); PDCD7(10081); |
| | | TSC2(7249); TRAF7(84231); CCNF(899); DNAJA3(9093); KATNB1(10300); |
| | | ELMO3(79767); PARD6A(50855); CDT1(81620); MAPK3(5595); DNAJA2 |
| | | (10294); FTS(64400); CSNK2A2(1459); TRADD(8717); SLC12A4 |
| | | (6560); SMPD3(55512); TERF2(7014); HIC1(3090); GSG2(83903); LIG3(3980); |
| | | CDC6(990); FZD2(2535); NME1(4830); PRKCA(5578); MAP2K6(5608); |
| | | ANAPC11(51529); NALP1(22861); GAS7(8522); BECN1(8678); BRCA1 |
| | | (672); HDAC5(10014); SEPT4(5414); ERN1(2081); CBX4(8535); MAPK4 |
| | | (5596); DCC(1630); ROCK1(6093); TXNL1(9352); BCL2(596); FZR1(51343); |
| | | HMG20B(10362); UHRF1(29128); PIN1(5300); DNM2(1785); PIK3R2 |
| | | (5296); UBA52(7311); NDUFA13(51079); PSMD8(5714); ARHGEF1(9138); |
| | | APOE(348); NALP2(55655); AURKC(6795); PRDX2(7001); DNASE2(1777); |
| | | SIRT2(22933); TGFB1(7040); PPP1R13L(10848); LIG1(3978); CARD8 |
| | | (22900); MCM8(84515); CEP250(11190); MYBL2(4605); PARD6B(84612); |
| | | E2F1(1869); DIDO1(11083); SON(6651); SIM2(6493); AGPAT3(56894); |
| | | HEMK2(29104); SNF1LK(150094); ITGB2(3689); BCL2L13(23786); CDC45L |
| | | (8318); SEPT5(5413); MCM5(4174); PPARA(5465); MAPK1(5594); PPM1F |
| | | (9647); SSTR3(6753); CARD10(29775); FLJ21687(79917); CCNB3 |
| | | (85417); TAF1(6872); NGFRAP1(27018); BIRC4(331); STAG2(10735); RP6 |

Table 4-51

| | | |
|---|---|---|
| | | -213H19.1(51765); DKC1(1736); SMC1L1(8243); BTK(695); SEPT6(23157); |
| | | CUL4B(8450); RP6-166C19.1(255313); PDCD8(9131); BCAP31(10134); |
| | | HCFC1(3054); |
| hsa-miR-422a | 84; 85; | PAX7(5081); NBL1(4681); NUDC(10726); RBBP4(5928); MACF1(23499); |
| hsa-miR-422b | | NFYC(4802); SH3GLB1(51100); BCL9(607); BNIPL(149428); C1orf56(54964); |
| | | UHMK1(127933); FASLG(356); TRAF5(7188); TGFB2(7042); FRAP1 |
| | | (2475); E2F2(1870); FAF1(11124); MCL1(4170); ASPM(259266); TP53BP2 |
| | | (7159); BIRC6(57448); HK2(3099); TCF7L1(83439); KCNIP3(30818); BRRN1 |
| | | (23397); ZAK(51776); CFLAR(8837); CASP10(843); ALS2CR19(117583); |
| | | YWHAQ(10971); PPM1G(5496); CARD12(58484); ERCC3(2071); WDR33 |
| | | (55339); TLK1(9874); ALS2CR15(130026); FZD5(7855); CUL3(8452); |
| | | HDAC4(9759); PDCD6IP(10015); TDGF1(6997); GNL3(26354); APPL |
| | | (26060); FBXO40(51725); PSMD2(5708); ZCSL2(285381); NEK4(6787); ATR |
| | | (545); CD38(952); HCAP-G(64151); EREG(2069); CCNG2(901); SPP1 |
| | | (6696); APBB2(323); LEF1(51176); VEGFC(7424); SKP2(6502); CARD6(84674); |
| | | APC(324); BNIP1(662); SEMA5A(9037); PRKAA1(5562); MCC(4163); |
| | | SEMA6A(57556); IRF1(3659); DUSP1(1843); E2F3(1871); PARC(23113); |
| | | FOXO3A(2309); TNFRSF21(27242); DST(667); BCLAF1(9774); MAP3K5 |
| | | (4217); PDCD2(5134); AHR(196); HDAC9(9734); STK17A(9263); EGFR |
| | | (1956); VKORC1L1(154807); HBP1(26959); CUL1(8454); CARD4(10392); |
| | | ELMO1(9844); SEMA3E(9723); CDK6(1021); FIS1(51024); ZC3HC1(51530); |
| | | HIPK2(28996); TNFRSF10C(8794); PTK2B(2185); ESCO2(157570); |
| | | UNC5D(137970); BAG4(9530); UBE2V2(7336); SULF1(23213); FZD6(8323); |
| | | PINX1(54984); LZTS1(11178); TNFRSF10B(8795); TNFRSF10A(8797); |
| | | CLU(1191); SFRP1(6422); MTSS1(9788); ANKRD15(23186); JAK2(3717); |
| | | DAPK1(1612); INVS(27130); C9orf28(89853); GPR107(57720); ABL1 |
| | | (25); PTPLAD2(401494); TAF1L(138474); GAS1(2619); SEMA4D(10507); |
| | | TNFSF15(9966); ANAPC2(29882); C10orf9(219771); PTEN(5728); HECTD2 |
| | | (143279); PDCD11(22984); BAG3(9531); IL2RA(3559); PARD3(56288); |
| | | FZD8(8325); DLG5(9231); DCLRE1A(9937); C10orf46(143384); TIAL1(7073); |
| | | BNIP3(664); STIM1(6786); HIPK3(10114); RPS6KB2(6199); LRP5(4041); |
| | | ATM(472); MAP3K11(4296); FZD4(8322); PPP2R1B(5519); CCND2 |
| | | (894); PTMS(5763); BCL2L14(79370); PDE1B(5153); APAF1(317); KNTC1 |
| | | (9735); TNFRSF1A(7132); LRP6(4040); FMNL3(91010); C12orf22(81566); |
| | | PCTK2(5128); IGF1(3479); HSMPP8(54737); FLJ40296(122183); FOXO1A |
| | | (2308); NEK3(4752); BCL2L2(599); PNN(5411); VASH1(22846); TRAF3 |
| | | (7187); GZMB(3002); CHES1(1112); CCNB2(9133); IGF1R(3480); TSC2 |
| | | (7249); DNASE1(1773); PAPD5(64282); CYLD(1540); E2F4(1874); GSPT1 |
| | | (2935); GGA2(23062); MAPK3(5595); DNAJA2(10294); SIAH1(6477); TERF2 |
| | | (7014); TXNL4B(54957); PAFAH1B1(5048); TNFSF12(8742); NTN1(9423); |
| | | CDC6(990); MAPT(4137); CROP(51747); PPM1D(8493); PRKCA(5578); |
| | | KPNA2(3838); MAP2K6(5608); SLC9A3R1(9368); MNT(4335); TP53 |
| | | (7157); GAS7(8522); SPAG5(10615); BRCA1(672); SEPT4(5414); GNA13(10672); |
| | | MAPRE2(10982); MAPK4(5596); ESCO1(114799); BCL2(596); CSNK1G2 |
| | | (1455); UHRF1(29128); DNM2(1785); MAP3K10(4294); PPP1R13L |
| | | (10848); CGB7(94027); PLCB1(23236); CEP250(11190); PARD6B(84612); |
| | | CSNK2A1(1457); PCNA(5111); E2F1(1869); SYCP2(10388); DIDO1(11083); |
| | | SON(6651); SEPT5(5413); MAPK1(5594); MN1(4330); SSTR3(6753); |
| | | HDAC6(10013); CCNB3(85417); TAF1(6872); SMC1L1(8243); SEPT6(23157); |
| | | HCFC1(3054); |
| hsa-miR-423 | 86; 764; 2254; | SAMD11(148398); VWA1(64856); KCNAB2(8514); C1orf188(148646); KIAA1026(23254); |
| | | EFHD2(79180); PDIK1L(149420); MACF1(23499); CTPS |
| | | (1503); C1orf164(55182); RAD54L(8438); CDKN2C(1031); CD2(914); FDPS |
| | | (2224); KIAA0446(9673); PPP1R12B(4660); TMCC2(9911); MTR(4548); |
| | | LOC440742(440742); NPHP4(261734); CTNNBIP1(56998); MAD2L2(10459); |
| | | ECE1(1889); MTF1(4520); TESK2(10420); KCNN3(3782); CLK2(1196); |
| | | GLT25D2(23127); ASPM(259266); PCNXL2(80003); FH(2271); AKT3 |
| | | (10000); HSPC159(29094); BCL2L11(10018); GYPC(2995); GPR39(2863); |
| | | IGFBP2(3485); COL4A3(1285); ALPPL2(251); ASB1(51665); SEPT2(4735); |
| | | ING5(84289); YWHAQ(10971); DNMT3A(1788); SLC30A3(7781); ZFP36L2 |
| | | (678); GPR75(10936); SERTAD2(9792); LOXL3(84695); TGOLN2 |
| | | (10618); STARD7(56910); FLJ10996(54520); ERCC3(2071); LYPD1(116372); |
| | | FLJ46347(389064); STAT1(6772); IGFBP5(3488); BHLHB2(8553); CAV3 |
| | | (859); VHL(7428); SLC22A13(9390); RBM5(10181); ARGFX(503582); TMEM111 |
| | | (55831); TMEM40(55287); ZCSL2(285381); ACAA1(30); NEK4( |

Table 4-52

6787); UBE1C(9039); IGSF11(152404); RAB6B(51660); STAG1(10274); PIK3CB
(5291); IGF2BP2(10644); HD(3064); LOC345222(345222); EVC(2121);
SLC25A4(291); STX18(53407); ACOX3(8310); FLJ23235(80008); APBB2
(323); PHOX2B(8929); TSPAN5(10098); LRP2BP(55805); PDCD6(10016);
CENPH(64946); PCDHB9(56127); UNC5A(90249); ATG12(9140); DNAJC18
(202052); PDE6A(5145); CAMK2A(815); KIAA1191(57179); TRIM7
(81786); E2F3(1871); C6orf25(80739); MAPK13(5603); PARC(23113);
VEGF(7422); SASH1(23328); SMOC2(64094); PRRT1(80863); TAPBP(6892);
CLIC5(53405); DST(667); COX7A2(1347); C6orf166(55122); PDSS2(57107);
DLL1(28514); RAC1(5879); AHR(196); STK17A(9263); ADCY1(107);
MGC33530(222008); ASB15(142685); LEP(3952); SMO(6608); MGC9712
(202915); SCRN1(9805); ELMO1(9844); IGFBP3(3486); WBSCR16(81554);
PDAP1(11333); GATS(352954); SLC37A3(84255); PRKAG2(51422);
DPYSL2(1808); PTK2B(2185); RIPK2(8767); MYC(4609); RHPN1(114822);
FAM86B1(85002); PSD3(23362); LOXL2(4017); DUSP4(1846); DKK4
(27121); PABPC1(26986); LYNX1(66004); ZNF16(7564); VLDLR(7436); RECK
(8434); DAPK1(1612); FGD3(89846); TMOD1(7111); FOXE1(2304); PALM2-
AKAP2(445815); C9orf28(89853); SLC27A4(10999); ODF2(4957);
SET(6418); UAP1L1(91373); CACNA1B(774); SEMA4D(10507); EPB41L4B
(54566); CDK5RAP2(55755); RAB14(51552); C9orf88(64855); C9orf140
(89958); CALML3(810); SEC61A2(55176); CREM(1390); SH2D4B(387694);
C10orf99(387695); BTRC(8945); SFMBT2(57713); NUDT5(11164); C10orf111
(221060); ITGB1(3688); RHOBTB1(9886); C10orf54(64115); DNAJB12
(54788); DLG5(9231); SFTPA2(6436); FER1L3(26509); SFRP5(6425);
FGF8(2253); FGFR2(2263); STIM1(6786); TRIM22(10346); EXT2(2132);
TSPAN18(90139); KIAA0652(9776); INCENP(3619); ESRRA(2101);
KLC2(64837); LRP5(4041); TPCN2(219931); FADD(8772); CUL5(8065);
NCAM1(4684); PKNOX2(63876); JAM3(83700); POLR2L(5441); TRIM68
(55128); DKK3(27122); MAP3K11(4296); CLCF1(23529); FLJ37266(283225);
CDON(50937); ADIPOR2(79602); HOXC12(3228); CDK2(1017); FZD10
(11211); NRIP2(83714); LRP6(4040); BCAT1(586); HDAC7A(51564); SLC11A2
(4891); CDK4(1019); CTDSP2(10106); GALNT4(8693); KCTD10
(83892); CAMKK2(10645); FLJ22471(80212); IL17D(53342); FGF9(2254);
PABPC3(5042); CDK8(1024); MGC9850(219404); FRY(10129); LOC440138
(440138); PHGDHL1(337867); COL4A2(1284); PROZ(8858); POSTN(10631);
DOCK9(23348); IRS2(8660); LOC440157(440157); C14orf132(56967);
TRAF3(7187); ACIN1(22985); KIAA1443(57594); TM9SF1(10548); L2HGDH
(79944); ERH(2079); CHES1(1112); AKT1(207); JAG2(3714); LOC440248
(440248); CCNDBP1(23582); RCN2(5955); CRTC3(64784); UBE3A
(7337); BCL2L10(10017); MYO5A(4644); ITGA11(22801); MGRN1(23295);
NUBP1(4682); PRRT2(112476); RNF40(9810); GPT2(84706); LONPL
(83752); SF3B3(23450); FLJ31875(197320); CDK10(8558); FBXL16(146330);
TXNDC11(51061); ERN2(10595); APPBP1(8883); LOC283849(283849);
SLC12A4(6560); NQO1(1728); WDR59(79726); KCNG4(93107); Pfs2
(51659); ZCCHC14(23174); MGC26885(124044); C16orf7(9605); GSG2(83903);
KIF1C(10749); MAPK7(5598); STARD3(10948); STAT5A(6776); NGFR
(4804); CACNG4(27092); KCTD2(23510); SEC14L1(6397); CHMP6(79643);
FOXK2(3607); RPH3AL(9501); CRK(1398); RILP(83547); SMYD4(114826);
ANKFY1(51479); NALP1(22861); GRAP(10750); JUP(3728); HDAC5
(10014); PECAM1(5175); RECQL5(9400); PTDSR(23210); C17orf70
(80233); C17orf62(79415); DTNA(1837); SLC14A1(6563); TNFRSF11A(8792);
NFATC1(4772); DLGAP1(9229); FZR1(51343); GIPC3(126326); MRPL4
(51073); FLJ25328(148231); GATAD2A(54815); FFAR1(2864); ARHGEF1
(9138); CACNG6(59285); FCAR(2204); ZNF471(57573); C19orf23(148046);
ZBTB7A(51341); PRSS15(9361); CD209(30835); ZNF490(57474);
HAPLN4(404037); POU2F2(5452); PPP1R13L(10848); DMPK(1760); FLJ10490
(55150); ZNF667(63934); TRIB3(57761); TTLL9(164395); POFUT1
(23509); ASXL1(171023); CEP250(11190); RAB22A(57403); VAPB(9217);
EDN3(1908); GTPBP5(26164); C20orf11(54994); ZGPAT(84619); NSFL1C
(55968); ZHX3(23051); RIMS4(140730); FLJ30313(253868); BTBD4(140685);
SIM2(6493); PDXK(8566); SEPT5(5413); LOC400891(400891); HIC2
(23119); UBE2L3(7332); KIAA0376(23384); LGALS1(3956); POLR2F
(5435); MPPED1(758); FLJ20699(55020); PANX2(56666); DGCR2(9993);
C22orf16(400916); MN1(4330); RP5-1119A7.4(80020); LOC400927(400927);
DNAL4(10126); CBX7(23492); SCUBE1(80274); INE1(8552); PCTK1

Table 4-53

| | | |
|---|---|---|
| | | (5127); HDAC6(10013); TRO(7216); NRK(203447); BIRC4(331); BCORL1 |
| | | (63035); PLXNTB3(5365); SLC35A2(7355); WNK3(65267); RAP2C(57826); |
| | | HS6ST2(90161); CD99L2(83692); HCFC1(3054); |
| hsa-miR-431 | 87; 765; 766; | SFN(2810); RBBP4(5928); MACF1(23499); RAP1A(5906); PEA15(8682); |
| | 767; | MDM4(4194); IL24(11009); TRAF5(7188); TGFB2(7042); CIAS1(114548); |
| | | GFI1(2672); NOTCH2(4853); CD5L(922); ASPM(259266); IL10(3586); RHOB |
| | | (388); PPP1CB(5500); BIRC6(57448); TCF7L1(83439); MAL(4118); BRRN1 |
| | | (23397); ZAK(51776); CFLAR(8837); CASP10(843); FZD7(8324); BOK |
| | | (666); CGREF1(10669); EIF2AK2(5610); ANAPC1(64682); IL1A(3552); |
| | | ERCC3(2071); MCM6(4175); STAT1(6772); STK17B(9262); FZD5(7855); |
| | | BARD1(580); FANCD2(2177); VHL(7428); PCAF(8850); RBM5(10181); NEK11 |
| | | (79858); CDCP1(64866); TUSC4(10641); HD(3064); FGF5(2250); PLK4 |
| | | (10733); GAK(2580); UNC5C(8633); CASP6(839); SFRP2(6423); NEK1 |
| | | (4750); VEGFC(7424); KIAA1909(153478); SKP2(6502); ERBB2IP(55914); |
| | | F2R(2149); APC(324); BNIP1(662); PRLR(5618); BIRC1(4671); PPP2CA |
| | | (5515); DNAJC18(202052); NRG2(9542); PDLIM7(9260); DDX41(51428); |
| | | CSNK2B(1460); C6orf69(222658); PARC(23113); VEGF(7422); FOXO3A |
| | | (2309); HECA(51696); SASH1(23328); MDC1(9656); DAXX(1616); IHPK3 |
| | | (117283); CUL7(9820); SUPT3H(8464); MCM3(4172); DST(667); SESN1(27244); |
| | | FRK(2444); SGK(6446); PLAGL1(5325); KATNA1(11104); AHR(196); |
| | | FZD1(8321); STAG3(10734); CASP2(835); MCM7(4176); ESCO2(157570); |
| | | RBPMS(11030); UBE2V2(7336); MYC(4609); WISP1(8840); BIN3(55909); |
| | | TNFRSF10A(8797); CLU(1191); DUSP4(1846); STK3(6788); RAD21 |
| | | (5885); DAPK1(1612); INVS(27130); CDK9(1025); BAG1(573); SEMA4D |
| | | (10507); TNFSF8(944); TRAF1(7185); TSC1(7248); DHTKD1(55526); FAS |
| | | (355); KIF11(3832); IL2RA(3559); PARD3(56288); FZD8(8325); DCLRE1A |
| | | (9937); BNIP3(664); HIPK3(10114); API5(8539); INCENP(3619); SIPA1 |
| | | (6494); ATM(472); LOH11CR2A(4013); RHOG(391); SESN3(143686); CASP1 |
| | | (834); BCL9L(283149); H2AFX(3014); PDE1B(5153); APAF1(317); FZD10 |
| | | (11211); HDAC7A(51564); CDK4(1019); DUSP6(1848); BRCA2(675); |
| | | RFP2(10206); TFDP1(7027); LATS2(26524); TXNTDC(81542); CGRRF1(10668); |
| | | ACIN1(22985); STRN3(29966); CDKL1(8814); PGF(5228); NEK9(91754); |
| | | BAG5(9529); JAG2(3714); CCNB2(9133); KIF23(9493); IGF1R(3480); |
| | | PDCD7(10081); DNASE1(1773); CARD15(64127); CTCF(10664); PKMYT1 |
| | | (9088); MAPK3(5595); DNAJA2(10294); SLC12A4(6560); GSG2(83903); |
| | | CDC6(990); FZD2(2535); PRR11(55771); PPM1D(8493); TLK2(11011); |
| | | NALP1(22861); GAS7(8522); PTRH2(51651); MALT1(10892); TXNL4A |
| | | (10907); FZR1(51343); ARHGEF1(9138); AES(166); CDKN2D(1032); DNASE2 |
| | | (1777); TGFB1(7040); ERCC2(2068); OPA3(80207); LIG1(3978); TRIB3 |
| | | (57761); MCM8(84515); PLCB1(23236); RBL1(5933); SYCP2(10388); USP16 |
| | | (10600); SON(6651); CHAF1B(8208); APP(351); CDC45L(8318); SMARCB1 |
| | | (6598); BID(637); PPM1F(9647); MN1(4330); PDGFB(5155); SMC1L2 |
| | | (27127); MAPK12(6300); BIRC4(331); ARHGEF6(9459); |
| hsa-miR-432* | 88; | TP73(7161); NBL1(4681); RCC1(1104); NFYC(4802); CDC20(991); SH3GLB1 |
| | | (51100); CDC7(8317); CDC14A(8556); BCL9(607); PEA15(8682); LAMC2 |
| | | (3918); TGFB2(7042); CIAS1(114548); FRAP1(2475); RCC2(55920); |
| | | E2F2(1870); YARS(8565); CLSPN(63967); FAF1(11124); BCL10(8915); GCLM |
| | | (2730); NOTCH2(4853); ASPM(259266); SYT2(127833); TP53BP2(7159); |
| | | PPP1CB(5500); BIRC6(57448); PRKCE(5581); HK2(3099); KCNIP3 |
| | | (30818); BRRN1(23397); RIF1(55183); TAIP-2(80034); ZAK(51776); HOXD10 |
| | | (3236); ALS2CR19(117583); PSMD1(5707); SEPT2(4735); TGOLN2 |
| | | (10618); BUB1(699); IL1A(3552); ERBB4(2066); BARD1(580); HDAC4(9759); |
| | | PDCD6IP(10015); APPL(26060); MCM2(4171); FXR1(8087); TP73L(8626); |
| | | CIDEC(63924); AXUD1(64651); NME6(10201); TRAIP(10293); TUSC4 |
| | | (10641); DNASE1L3(1776); UBE1C(9039); PROK2(60675); STAG1(10274); |
| | | PIK3CB(5291); CCNG2(901); FGF5(2250); FGF2(2247); PLK4(10733); |
| | | APBB2(323); DKK2(27123); SFRP2(6423); NIPBL(25836); CARD6(84674); |
| | | F2R(2149); APC(324); PCDHGA1(56114); BNIP1(662); UNC5A(90249); |
| | | MCC(4163); ATG12(9140); SEMA6A(57556); IRF1(3659); NME5(8382); |
| | | CD14(929); DDX41(51428); RIPK1(8737); PARC(23113); VEGF(7422); |
| | | IL17(3605); PTP4A1(7803); FOXO3A(2309); TNFAIP3(7128); HECA(51696); |
| | | NEDD9(4739); DHX16(8449); CCND3(896); SUPT3H(8464); MCM3 |
| | | (4172); DST(667); FRK(2444); TSPYL4(23270); LATS1(9113); AHR(196); EGFR |
| | | (1956); RINT1(60561); TRIAD3(54476); IGFBP3(3486); CDK6(1021); |
| | | MCM7(4176); HIPK2(28996); BRAF(673); FASTK(10922); TNFRSF10C( |

Table 4-54

| | | |
|---|---|---|
| | | 8794); BNIP3L(665); PTK2B(2185); SULF1(23213); EBAG9(9166); PINX1 |
| | | (54984); TNFRSF10B(8795); SFRP1(6422); TP53INP1(94241); CDC37L1 |
| | | (55664); INVS(27130); ABL1(25); TRAF2(7186); TAF1L(138474); SEMA4D |
| | | (10507); PTCH(5727); TNFSF8(944); TRAF1(7185); CIZ1(25792); TSC1 |
| | | (7248); PTEN(5728); PDCD11(22984); DOCK1(1793); CUL2(8453); DLG5 |
| | | (9231); FRAT2(23401); FBXW4(6468); LGALS12(85329); SSSCA1(10534); |
| | | LRP5(4041); CCND1(595); LOH11CR2A(4013); EI24(9538); HRAS(3265); |
| | | DKK3(27122); MAP3K11(4296); CLCF1(23529); GAB2(9846); FZD4 |
| | | (8322); SESN3(143686); BTG4(54766); PPP2R1B(5519); BCL9L(283149); |
| | | CCND2(894); LTBR(4055); TNFRSF7(939); CNAP1(9918); PTPN6(5777); |
| | | TEGT(7009); ESPL1(9700); PDE1B(5153); RBMS2(5939); APAF1(317); LOC196463 |
| | | (196463); FZD10(11211); NOL1(4839); LRP6(4040); MCRS1(10445); |
| | | FAIM2(23017); TNFRSF19(55504); LATS2(26524); PNN(5411); CGRRF1 |
| | | (10668); VASH1(22846); GZMH(2999); GZMB(3002); STRN3(29966); |
| | | LTBP2(4053); CHES1(1112); AKT1(207); IGF1R(3480); BCL2L10(10017); |
| | | CSNK1G1(53944); FLJ39743(283777); RAB40C(57799); CRAMP1L(57585); |
| | | TSC2(7249); CCNF(899); CARD15(64127); CYLD(1540); RBL2(5934); |
| | | KIAA0513(9764); KIAA0182(23199); TRADD(8717); SLC12A4(6560); |
| | | TERF2(7014); TXNL4B(54957); PCOLN3(5119); ALOX15B(247); TRAF4 |
| | | (9618); LIG3(3980); YPEL2(388403); TLK2(11011); PRKCA(5578); SPHK1 |
| | | (8877); ANAPC11(51529); NALP1(22861); TP53(7157); GAS2L2(246176); |
| | | BECN1(8678); HDAC5(10014); CBX4(8535); CABLES1(91768); DCC(1630); |
| | | MALT1(10892); BCL2(596); CSNK1G2(1455); CHAF1A(10036); UHRF1 |
| | | (29128); PIK3R2(5296); CCNE1(898); PLEKHG2(64857); APOE(348); |
| | | FOSB(2354); SPHK2(56848); AURKC(6795); SIRT2(22933); KLK10(5655); |
| | | LOC284296(284296); MCM8(84515); CEP250(11190); STK4(6789); UBE2C |
| | | (11065); CD40(958); BIRC7(79444); CSNK2A1(1457); BCL2L1(598); |
| | | C20orf112(140688); E2F1(1869); GGTL3(2686); DIDO1(11083); SON(6651); |
| | | MX1(4599); APP(351); SNF1LK(150094); ADORA2A(135); NF2(4771); |
| | | MCM5(4174); EP300(2033); BID(637); MAPK1(5594); PPM1F(9647); MN1 |
| | | (4330); CARD10(29775); TOB2(10766); HDAC6(10013); CCNB3(85417); |
| | | TAF1(6872); BIRC4(331); STAG2(10735); FIGF(2277); SMC1L1(8243); ARHGEF6 |
| | | (9459); HCFC1(3054); |
| hsa-miR-433 | 89; 768; 769; | TNFRSF1B(7133); NUDC(10726); MACF1(23499); SH3GLB1(51100); SLC30A7 |
| | | (148867); PROK1(84432); RAP1A(5906); BCL9(607); UHMK1(127933); |
| | | PTPRC(5788); IL19(29949); TRAF5(7188); DISC1(27186); CIAS1(114548); |
| | | MUTYH(4595); RAB3B(5865); SASS6(163786); ASPM(259266); PPP1CB |
| | | (5500); BIRC6(57448); CCT7(10574); KCNIP3(30818); BRRN1(23397); |
| | | PDCL3(79031); ZC3H6(376940); RIF1(55183); TAIP-2(80034); FZD7 |
| | | (8324); COL4A3(1285); PSMD1(5707); CYP1B1(1545); TIA1(7072); C2orf3 |
| | | (6936); BUB1(699); IFIH1(64135); TLK1(9874); FRZB(2487); VHL(7428); |
| | | PCAF(8850); PDCD6IP(10015); RBM5(10181); APPL(26060); ACPP(55); |
| | | SMC4L1(10051); KNG1(3827); CIDEC(63924); TUSC4(10641); NEK4(6787); |
| | | CD200R1(131450); GSK3B(2932); STAG1(10274); ATR(545); SLAH2 |
| | | (6478); HD(3064); HCAP-G(64151); SEPT11(55752); FGF5(2250); APBB2 |
| | | (323); CCNI(10983); CASP6(839); MAD2L1(4085); SLC7A11(23657); SFRP2 |
| | | (6423); SORBS2(8470); LOC91137(91137); RAD50(10111); PRLR(5618); |
| | | RPL37(6167); MCC(4163); SEMA6A(57556); IRF1(3659); PPP2CA(5515); |
| | | NME5(8382); CDC25C(995); PFDN1(5201); DUSP1(1843); RIPK1(8737); |
| | | E2F3(1871); PTP4A1(7803); TTK(7272); SASH1(23328); NEDD9(4739); |
| | | BAK1(578); CUL7(9820); DST(667); KATNA1(11104); LATS1(9113); AHR |
| | | (196); STK17A(9263); FZD9(8326); FZD1(8321); BCAP29(55973); CUL1(8454); |
| | | PMS2(5395); CYCS(54205); ELMO1(9844); SFRP4(6424); YWHAG |
| | | (7532); PTK2B(2185); ESCO2(157570); SULF1(23213); RIPK2(8767); TNFRSF10D |
| | | (8793); ADRA1A(148); TRIM35(23087); DUSP4(1846); RAD21(5885); |
| | | ANKRD15(23189); JAK2(3717); UNC13B(10497); DAPK1(1612); IFNW1 |
| | | (3467); IFNA2(3440); CDKN2B(1030); BAG1(573); CTNNAL1(8727); |
| | | TRAF1(7185); PPP6C(5537); TSC1(7248); ZMYND11(10771); DKK1(22943); |
| | | SIRT1(28411); SGPL1(8879); FAS(355); BTRC(8945); PDCD11(22984); |
| | | CSPG6(9126); TCF7L2(6934); DOCK1(1793); PARD3(56288); CUL2(8453); |
| | | PPP3CB(5532); KCNMA1(3778); DLG5(9231); GFRA1(2674); C10orf46 |
| | | (143384); MKI67(4288); STIM1(6786); WEE1(7465); BIRC3(380); ATM |
| | | (472); LOH11CR2A(4013); CHEK1(1111); RELA(5970); MTL5(9633); FZD4 |
| | | (8322); SESN3(143686); CASP5(838); BTG4(54766); CCND2(894); CNAP1 |
| | | (9918); ESPL1(9700); MDM2(4193); GAS2L3(283431); TMEM132B(11 |

Table 4-55

| | | |
|---|---|---|
| | | 4795); FGF6(2251); PCTK2(5128); MPHOSPH9(10198); SCARB1(949); FGF9 |
| | | (2254); CDK8(1024); BRCA2(675); RB1(5925); CUL4A(8451); NEK3 |
| | | (4752); PSEN1(5663); EGLN3(112399); NEK9(91754); CHES1(1112); BAG5 |
| | | (9529); PPP1R13B(23368); FGF7(2252); MAPK6(5597); TPM1(7168); TRAF7 |
| | | (84231); PAPD5(64282); CARD15(64127); CTCF(10664); CDT1(81620); |
| | | AXIN1(8312); NME3(4832); GSPT1(2935); DNAJA2(10294); FTS(64400); |
| | | TXNL4B(54957); ALOX12(239); MAPK7(5598); NF1(4763); FZD2(2535); |
| | | CROP(51747); PRKCA(5578); KPNA2(3838); GAS7(8522); BRCA1(672); |
| | | CETN1(1068); MAPRE2(10982); DCC(1630); MALT1(10892); PHLPP(23239); |
| | | BCL2(596); TXNL4A(10907); TNFSF9(8744); ZNF440(126070); NDUFA13 |
| | | (51079); BAX(581); ATF5(22809); TNFSF14(8740); ZNF443(10224); |
| | | MCM8(84515); STK4(6789); CSE1L(1434); CSNK2A1(1457); RBL1(5933); |
| | | SYCP2(10388); DIDO1(11083); IFNAR1(3454); NF2(4771); EP300(2033); |
| | | MAPK1(5594); SMC1L2(27127); MAPK12(6300); HDAC6(10013); CCNB3 |
| | | (85417); TAF1(6872); NGFRAP1(27018); STAG2(10735); SMC1L1 |
| | | (8243); SPIN2(54466); CUL4B(8450); CETN2(1069); |
| hsa-miR-449 | 90; 770; 771; | VWA1(64856); H6PD(9563); NBL1(4681); PLA2G2F(64600); SESN2(83667); |
| | 772; 773; | HDAC1(3065); MACF1(23499); CDC20(991); PLK3(1263); BCL9(607); |
| | 774; 775; 776; | HCN3(57657); PEA15(8682); FASLG(356); NAV1(89796); ADORA1(134); |
| | 917; 918; | MDM4(4194); CIAS1(114548); TNFRSF14(8764); TNFRSF9(3604); CTNNBIP1 |
| | 2255; | (56998); DFFA(1676); FRAP1(2475); CASP9(842); ZBTB17(7709); |
| | | RCC2(55920); C1orf183(55924); NOTCH2(4853); PYGO2(90780); TP53BP2 |
| | | (7159); KCNK3(3777); BIRC6(57448); PRKCE(5581); SEMA4F(10505); |
| | | HK2(3099); MAL(4118); BRRN1(23397); TAIP-2(80034); CFLAR(8837); |
| | | FZD7(8324); CD28(940); ASB1(51665); TGFA(7039); EDAR(10913); PAX3 |
| | | (5077); CUL3(8452); HDAC4(9759); DTYMK(1841); FANCD2(2177); VHL |
| | | (7428); DLEC1(9940); MCM2(4171); CLDN18(51208); SMC4L1(10051); CMYA1 |
| | | (165904); UCN2(90226); NEK4(6787); RAB43(339122); SLCO2A1 |
| | | (6578); PIK3CB(5291); TFDP2(7029); HD(3064); ANAPC4(29945); EREG |
| | | (2069); SEPT11(55752); NFKB1(4790); FGF2(2247); LETM1(3954); MXD4 |
| | | (10608); SNCA(6622); UNC5C(8633); LEF1(51176); CASP6(839); KIAA1909 |
| | | (153478); CCNB1(891); CSNK1G3(1456); TCF7(6932); NDST1(3340); |
| | | CPLX2(10814); UNC5A(90249); TPPP(11076); PRLR(5618); BIRC1(4671); |
| | | SEMA6A(57556); IRF1(3659); PPP2CA(5515); CDC23(8697); CSNK1A1 |
| | | (1452); DUSP1(1843); E2F3(1871); TCF19(6941); CSNK2B(1460); MAPK13 |
| | | (5603); PARC(23113); VEGF(7422); IL17(3605); TNFAIP3(7128); HECA |
| | | (51696); NEDD9(4739); DHX16(8449); DAXX(1616); CUL7(9820); DST |
| | | (667); ATG5(9474); FRK(2444); PLAGL1(5325); DLL1(28514); FZD9(8326); |
| | | BCAP29(55973); CASP2(835); MAD1L1(8379); TRIAD3(54476); SOSTDC1 |
| | | (25928); CYCS(54205); CARD4(10392); ELMO1(9844); YWHAG(7532); |
| | | CDK6(1021); MCM7(4176); ZC3HC1(51530); MGC5242(78996); HIPK2 |
| | | (28996); KIAA0773(9715); POLR3D(661); TNFRSF10C(8794); UNC5D(137970); |
| | | SULF1(23213); E2F5(1875); PINIX1(54984); C8orf13(83648); LZTS1 |
| | | (11178); TNFRSF10B(8795); TRIM35(23087); DUSP4(1846); MOS(4342); |
| | | CCNE2(9134); TP53INP1(94241); RAD21(5885); EXT1(2131); JAK2(3717); |
| | | UNC13B(10497); RECK(8434); C9orf47(286223); GADD45G(10912); |
| | | FGD3(89846); ABL1(25); TAF1L(138474); GAS1(2619); SEMA4D(10507); |
| | | PTCH(5727); TRAF1(7185); CIZ1(25792); ZMYND11(10771); DKK1(22943); |
| | | SGPL1(8879); UNC5B(219699); FRAT1(10023); LZTS2(84445); BTRC |
| | | (8945); PDCD11(22984); BAG3(9531); DOCK1(1793); IL2RA(3559); DLG5 |
| | | (9231); SFRP5(6425); FBXW4(6468); FAM26C(255022); DCLRE1A(9937); |
| | | MKI67(4288); WEE1(7465); TSPAN18(90139); F2(2147); INCENP(3619); |
| | | DPF2(5977); SIPA1(6494); RPS6KB2(6199); ATM(472); CD3E(916); LOH11CR2A |
| | | (4013); EI24(9538); DUSP8(1850); IGF2(3481); CDKN1C(1028); |
| | | RHOG(391); BAD(572); CDCA5(113130); MAP3K11(4296); RELA(5970); |
| | | FOSL1(8061); CLCF1(23529); PPP1CA(5499); FZD4(8322); BTG4(54766); |
| | | PPP2R1B(5519); PCSK7(9159); BCL9L(283149); LTBR(4055); TNFRSF7 |
| | | (939); BCL2L14(79370); CDKN1B(1027); IAPP(3375); ESPL1(9700); |
| | | PDE1B(5153); TNFRSF1A(7132); NOL1(4839); BCAT1(586); HDAC7A(51564); |
| | | MCRS1(10445); FMNL3(91010); FAIM2(23017); C12orf22(81566); |
| | | DDIT3(1649); PCTK2(5128); PPP1CC(5501); ANAPC5(51433); DIABLO |
| | | (56616); VPS37B(79720); MPHOSPH9(10198); CHFR(55743); RFR2(10206); |
| | | NEK3(4752); GZMH(2999); STRN3(29966); EGLN3(112399); NFKBIA |
| | | (4792); LTBP2(4053); TGFB3(7042); CHES1(1112); MOAP1(64112); JAG2 |
| | | (3714); THSD4(79875); CSK(1445); IGF1R(3480); NDN(4692); BMF(9042 |

Table 4-56

| | | |
|---|---|---|
| | | 7); DAPK2(23604); CSNK1G1(53944); BCL2A1(597); FLJ22795(80154); AP3S2 |
| | | (10239); FLJ36208(283948); RAB40C(57799); TRAF7(84231); KREMEN2 |
| | | (79412); DNASE1(1773); PLK1(5347); CARD15(64127); CCL22(6367); |
| | | KATNB1(10300); NOL3(8996); CTCF(10664); VPS4A(27183); CDT1 |
| | | (81620); AFG3L1(172); GAS8(2622); PKMYT1(9088); XYLT1(64131); ERN2 |
| | | (10595); SEPT1(1731); C16orf58(64755); DNAJA2(10294); FTS(64400); |
| | | SMPD3(55512); RUTBC1(9905); GSG2(83903); ALOX12(239); TNFSF12 |
| | | (8742); ALOX15B(247); NTN1(9423); SARM1(23098); TRAF4(9618); NF1 |
| | | (4763); LIG3(3980); STARD3(10948); FZD2(2535); MAPT(4137); NGFR(4804); |
| | | PRKCA(5578); CDK3(1018); BIRC5(332); ANAPC11(51529); GPS1(2873); |
| | | MNT(4335); NALP1(22861); GPS2(2874); TP53(7157); VAMP2(6844); |
| | | FLOT2(2319); MPP2(4355); ERN1(2081); SOCS3(9021); CABLES1(91768); |
| | | MAPRE2(10982); KIAA0427(9811); SERPINB2(5055); DSC3(1825); |
| | | FZR1(51343); HMG20B(10362); CHAF1A(10036); CASP14(23581); PIK3R2 |
| | | (5296); CCNE1(898); LGALS7(3963); SAMD4B(55095); MAP3K10(4294); |
| | | AXL(558); FOSB(2354); GRLF1(2909); SPHK2(56848); BAX(581); PPP2R1A |
| | | (5518); NALP2(55655); FLJ45684(400666); TNFSF14(8740); CDC37 |
| | | (11140); DPF1(8193); DEDD2(162989); ERCC2(2068); PPP1R13L(10848); |
| | | LIG1(3978); LOC284296(284296); TRIB3(57761); STK35(140901); PLCB1 |
| | | (23236); SRC(6714); MYBL2(4605); CD40(958); PARD6B(84612); ProSAPiP1 |
| | | (9762); CTCFL(140690); CABLES2(81928); DIDO1(11083); USP16 |
| | | (10600); SON(6651); PDXK(8566); BCL2L13(23786); SEPT5(5413); NF2 |
| | | (4771); MTP18(51537); PGEA1(25776); TNRC6B(23112); EP300(2033); SEPT3 |
| | | (55964); BIK(638); MAPK8IP2(23542); BID(637); MAPK1(5594); PPM1F |
| | | (9647); IL2RB(3560); SSTR3(6753); CARD10(29775); TOB2(10766); |
| | | POLR3H(171568); SMC1L2(27127); HDAC6(10013); CCNB3(85417); MAGEH1 |
| | | (28986); TAF1(6872); IKBKG(8517); SMC1L1(8243); HCFC1(3054); |
| hsa-miR-451 | 91; 777; 778; | YTHDF2(51441); CDC20(991); FAM73A(374986); SPATA1(64173); CYB561D1 |
| | 779; 780; | (284613); VANGL1(81839); IL6R(3570); CD1D(912); BAT2D1(23215); |
| | 781; 782; | PAPPA2(60676); NR5A2(2494); TRAF5(7188); DISC1(27185); SSU72(29101); |
| | | CTNNBIP1(56998); ZNF644(84146); GFI1(2672); GPATC4(54865); |
| | | LZTR2(89866); ZNF281(23528); MYOG(4656); C1orf145(574407); ZNF496 |
| | | (84838); KLF11(8462); LPIN1(23175); IL18R1(8809); FLJ37440(129804); |
| | | IMP4(92856); CTDSP1(58190); CAB39(51719); B3GNT7(93010); OSR1 |
| | | (130497); PREPL(9581); PNPT1(87178); RAB1A(5861); EIF2AK3(9451); |
| | | MGC4268(83607); LOC130074(130074); TTC21B(79809); SPBC25(57405); |
| | | ATF2(1386); TTN(7273); ACADL(33); THAP4(51078); RAB5A(5868); CRTAP |
| | | (10491); MOBP(4336); XNF654(55279); HHLA2(11148); ZDHHC23 |
| | | (254887); C3orf1(51300); DTX3L(151636); GMPS(8333); LPP(4026); OXTR |
| | | (5021); RAFTLIN(23180); GADL1(339896); CMTM6(54918); RASSF1(11186); |
| | | ARHGEF3(50650); PRICKLE2(166336); DCBLD2(131566); PLSCR2 |
| | | (57047); IGSF10(285313); PDCD10(11235); LSG1(55341); CD38(952); |
| | | LPHN3(23284); EREG(2069); AFAP(60312); USP46(64854); UBE1L2(55236); |
| | | GRSF1(2926); CXXC4(80319); FAM105A(54491); KIF2(3796); GRAMD3 |
| | | (65983); MFAP3(4238); SFXN1(94081); MAML1(9794); SEMA5A(9037); |
| | | OXCT1(5019); PDE4D(5144); BIRC1(4671); ST8SIA4(7903); DNAJC18 |
| | | (202052); DPYSL3(1809); KIAA0676(23061); C6orf151(154007); BMP6 |
| | | (654); LOC221711(221711); LOC401252(401262); POU3F2(5454); TCBA1 |
| | | (154215); ZDHHC14(79683); SYNJ2(8871); NEDD9(4739); PSMB8(5696); |
| | | TAPBP(6892); BAK1(578); TNFRSF21(27242); DST(667); BACH2(60468); |
| | | TXLNB(167838); PDCD2(5134); ZNRF2(223082); DKFZp761I2123(83637); |
| | | SVH(83787); CAV1(857); ZNF398(57541); MEOX2(4223); TARP(445347); |
| | | HIP1(3092); AKR1B1(231); KIAA1456(57604); SLC39A14(23516); |
| | | FNTA(2339); YTHDF3(253943); CPNE3(8895); PTDSS1(9791); FAM91A1 |
| | | (157769); PHF20L1(51105); BIN3(55909); STC1(6781); NSMAF(8439); |
| | | MSC(9242); ZNF704(619279); YWHAZ(7534); SMARCA2(6595); PCSK5 |
| | | (5125); INVS(27130); FAM73B(84895); CDKN2B(1030); C9orf48(347240); |
| | | APBA1(320); TNFSF15(9966); ASTN2(23245); RAB14(51552); FNBP1(23048); |
| | | UCK1(83549); TSC1(7248); MGC61598(441478); SEC23IP(11196); |
| | | ACADSB(36); GPR26(2849); BCCIP(56647); NUDT5(11164); C10orf111(221060); |
| | | RASGEF1A(221002); SPOCK2(9806); SYNPO2L(79933); SFXN4 |
| | | (119559); SWAP70(23075); LUZP2(338645); EHF(26298); STX3A(6809); |
| | | C11orf30(56946); ALDH3B2(222); MTL5(9633); HBXAP(51773); SRPR(6734); |
| | | CACNA1C(775); C12orf5(57103); NTF3(4908); CLEC2D(29121); AE |

Table 4-57

| | | |
|---|---|---|
| | | BP2(121536); PFDN5(5204); CUTL2(23316); ZNF140(7699); DUSP16(80824); |
| | | DBX2(440097); FMNL3(91010); FAIM2(23017); CDK4(1019); OSBPL8 |
| | | (114882); NT5DC3(51559); KCTD10(83892); WDFY2(115825); UPF3A |
| | | (65110); SLITRK1(114798); ATP4B(496); BATF(10538); EVL(51466); CRIP2 |
| | | (1397); EGLN3(112399); SPTB(6710); WDR22(8816); FBLN5(10516); |
| | | KLF13(51621); CAPN3(825); GALK2(2585); FAM63B(54629); NIPA1(123606); |
| | | ZFP106(64397); TTBK2(146057); ARPP-19(10776); ADAM10(102); |
| | | KIAA0101(9768); CALML4(91860); NPTN(27020); C15orf40(123207); PMM2 |
| | | (5373); PRO0149(29035); ADCY7(113); CYLD(1540); KIAA0174(9798); |
| | | RPL13(6137); GRIN2A(2903); FTS(64400); SLC7A6OS(84138); M-RIP |
| | | (23164); C17orf39(79018); DRG2(1819); CPD(1362); PSMD11(5717); LIG3 |
| | | (3980); DDX42(11325); SLC16A3(9123); CXCL16(58191); DHX33(56919); |
| | | ACACA(31); TMEM105(284186); LIPG(9388); ROCK1(6093); AQP4(361); |
| | | RKHD2(51320); ZADH2(284273); MBP(4155); SCAMP4(113178); CHAF1A |
| | | (10036); GCDH(2639); F2RL3(9002); KIAA0892(23383); GATAD2A |
| | | (54815); SAMD4B(55095); PLEKHG2(64857); LENG8(114823); ZNF272 |
| | | (10794); PRG2(79948); CDKN2D(1032); ERF(2077); ERCC2(2068); KIAA1219 |
| | | (57148); KCNK15(60598); STK4(6789); PCNA(5111); VSX1(30813); CBLN4 |
| | | (140689); ATP5E(514); SYCP2(10388); C21orf63(59271); PDXK(8566); |
| | | ADARB1(104); BAGE(574); C21orf91(54149); C21orf129(150135); PTTG1IP |
| | | (754); POFUT2(23275); LSS(4047); TBX1(6899); UBE2L3(7332); MIF |
| | | (4282); EP300(2033); PPARA(5465); CRELD2(79174); CECR5(27440); |
| | | BID(637); DGCR2(9993); DGCR13(26221); C22orf23(84645); SMC1L2(27127); |
| | | CELSR1(9620); CERK(64781); MSL3L1(10943); CA5B(11238); GK |
| | | (2710); ZNF81(347344); OGT(8473); RP6-213H19.1(51765); ZIC3(7547); PNMA6A |
| | | (84968); GDI1(2664); ZBED1(9189); FAM9C(171484); CXorf21(80231); |
| | | DDX3Y(8653); |
| hsa-miR-452 | 92; 783; | PRKCZ(5590); UTP11L(51118); MACF1(23499); NFYC(4802); CDC14A(8556); |
| | | IL24(11009); TRAF5(7188); TGFB2(7042); TNFRSF18(8784); TNFRSF9 |
| | | (3604); TESK2(10420); BCL10(8915); NOTCH2(4853); CD5L(922); TNFSF18 |
| | | (8995); BIRC6(57448); GKN1(56287); ZAK(51776); CFLAR(8837); |
| | | CASP8(841); SEPT2(4735); SEPT10(151011); IL1A(3552); ERCC3(2071); |
| | | RAPH1(65059); BARD1(580); HDAC4(9759); PCAF(8850); MLH1(4292); |
| | | APPL(26060); MRAS(22808); SENP2(59343); KNG1(3827); TP73L(8626); |
| | | TADA3L(10474); RASSF1(11186); NEK4(6787); STAG1(10274); ATR(545); |
| | | HD(3064); HCAP-G(64151); IL8(3576); CCNG2(901); SPP1(6696); FGF2 |
| | | (2247); USP46(64854); UNC5C(8633); DKK2(27123); ANAPC10(10393); |
| | | NEK1(4750); NIPBL(25836); ERBB2IP(55914); RAD50(10111); UNC5A |
| | | (90249); PRLR(5618); UNG2(10309); CDC23(8697); CDC25C(995); FGF1 |
| | | (2246); RNF130(55819); E2F3(1871); CDC5L(988); BAG2(9532); PTP4A1 |
| | | (7803); TTK(7272); FOXO3A(2309); HECA(51696); DST(667); SESN1(27244); |
| | | SGK(6446); LATS1(9113); SEPT7(989); DKFZp761I2123(83637); ELMO1 |
| | | (9844); CDK6(1021); HIPK2(28996); ESCO2(157570); UNC5D(137970); |
| | | UBE2V2(7336); TNFRSF10A(8797); DUSP4(1846); TP53INP1(94241); |
| | | TNFRSF11B(4982); RECK(8434); BNC2(54796); CDKN2B(1030); BAG1 |
| | | (573); PTCH(5727); CTNNAL1(8727); TNFSF8(944); DCLRE1A(9937); |
| | | MKI67(4288); HIPK3(10114); BIRC3(330); ATM(472); DIXDC1(85458); CHEK1 |
| | | (1111); DKK3(27122); FZD4(8322); SESN3(143686); ZW10(9183); CCT2 |
| | | (10576); FZD10(11211); LRP6(4040); HDAC7A(51564); PPP1CC(5501); |
| | | HSMPP8(54737); RFP2(10206); LIG4(3981); GPR65(8477); TRAF3(7187); |
| | | NEK9(91754); TGFB3(7043); CHES1(1112); TCL1A(8115); BAG5(9529); |
| | | PPP1R13B(23368); KIF23(9493); IGF1R(3480); AVEN(57099); SLC12A6 |
| | | (9990); PYGO1(26108); BCL2A1(597); TRAF7(84231); CCNF(899); DNASE1 |
| | | (1773); ELMO3(79767); GRIN2A(2903); GSPT1(2935); DNAJA2(10294); |
| | | HIC1(3090); PAFAH1B1(5048); NF1(4763); NGFR(4804); PRKCA(5578); |
| | | MAP2K6(5608); P2RX1(5023); NALP1(22861); BECN1(8678); BRCA1 |
| | | (672); PHB(5245); SEPT4(5414); MAPRE2(10982); SETBP1(26040); MAPK4 |
| | | (5596); DCC(1630); ESCO1(114799); ZNF443(10224); CARD8(22900); |
| | | PLCB1(23236); MAPRE1(22919); CSE1L(1434); BIRC7(79444); RBL1 |
| | | (5933); CTCFL(140690); CABLES2(81928); USP16(10600); SON(6651); CDC45L |
| | | (8318); SEPT5(5413); PGEA1(25776); EP300(2033); MAPK8IP2(23542); |
| | | MAPK1(5594); MN1(4330); RP6-213H19.1(51765); SPIN2(54466); |
| | | OPHN1(4983); CUL4B(8450); ARHGEF6(9459); |
| hsa-miR-452* | 93; | H6PD(9563); TNFRSF1B(7133); CDC42(998); SFN(2810); ZCCHC17(51538); |
| | | KHDRBS1(10657); RBBP4(5928); MACF1(23499); CDC20(991); PLK |

Table 4-58

| | | |
|---|---|---|
| | | 3(1263); CDKN2C(1031); SH3GLB1(51100); DAP3(7818); PEA15(8682); |
| | | ADORA1(134); IL24(11009); GOS2(50486); TNFRSF14(8764); DFFA(1676); |
| | | FRAP1(2475); RCC2(55920); E2F2(1870); CLSPN(63967); TESK2(10420); |
| | | GFI1(2672); NOTCH2(4853); DEDD(9191); ASPM(259266); PIK3C2B |
| | | (5287); RAB7L1(8934); TP53BP2(7159); MAPRE3(22924); BIRC6(57448); |
| | | PRKCE(5581); MSH2(4436); HTRA2(27429); KCNIP3(30818); BRRN1 |
| | | (23397); PDCL3(79031); ZAK(51776); CASP8(841); FZD7(8324); NRP2(8828); |
| | | CAPN10(11132); PPM1G(5496); RTN4(57142); ANAPC1(64682); FRZB |
| | | (2487); PAX3(5077); VHL(7428); ZNF621(285268); TDGF1(6997); APPL |
| | | (26060); NEK11(79858); PSMD2(5708); TADA3L(10474); MRPS25(64432); |
| | | TRAIP(10293); DNASE1L3(1776); UBE1C(9039); SLCO2A1(6578); HD |
| | | (3064); CD38(952); HCAP-G(64151); CCNG2(901); GAK(2580); APBB2(323); |
| | | CCNI(10983); DKK2(27123); NIPBL(25836); CARD6(84674); ERBB2IP |
| | | (55914); F2R(2149); APC(324); DCP2(167227); TCF7(6932); GALNT10 |
| | | (55568); PRLR(5618); IRF1(3659); PPP2CA(5515); FGF1(2246); CSNK1A1 |
| | | (1452); E2F3(1871); CSNK2B(1460); MAPK13(5603); PARC(23113); FOXO3A |
| | | (2309); TNFAIP3(7128); SASH1(23328); NEDD9(4739); DHX16(8449); |
| | | MDC1(9656); BAK1(578); SUPT3H(8464); TNFRSF21(27242); DST(667); |
| | | BCLAF1(9774); MAP3K5(4217); PLAGL1(5325); STK17A(9263); EGFR |
| | | (1956); FZD1(8321); BCAP29(55973); CASP2(835); CUL1(8454); MAD1L1 |
| | | (8379); IGFBP3(3486); FIS1(51024); ZC3HC1(51530); PTN(5764); HIPK2 |
| | | (28996); BRAF(673); GIMAP6(474344); TNFRSF10C(8794); ESCO2 |
| | | (157570); UBE2V2(7336); FZD6(8323); EBAG9(9166); MYC(4609); LZTS1 |
| | | (11178); TNFRSF10D(8793); DUSP4(1846); TP53INP1(94241); ANKRD15 |
| | | (23189); DAPK1(1612); GADD45G(10912); INVS(27130); EXOSC2(23404); |
| | | TRAF2(7186); MOBKL2B(79817); BAG1(573); SEMA4D(10507); PTCH |
| | | (5727); CTNNAL1(8727); TNFSF15(9966); TRAF1(7185); ASB6(140459); |
| | | TSC1(7248); SIRT1(23411); SGPL1(8879); UNC5B(219699); KIF11(3832); |
| | | FRAT1(10023); NOLC1(9221); SUFU(51684); NEURL(9148); DOCK1 |
| | | (1793); IL2RA(3559); DLG5(9231); FBXW4(6468); C10orf46(143384); MKI67 |
| | | (4288); WEE1(7465); HIPK3(10114); LGALS12(85329); VEGFB(7423); |
| | | FADD(8772); AMOTL1(154810); CUL5(8065); ATM(472); CD3G(917); LOH11CR2A |
| | | (4013); DKK3(27122); HRASLS3(11145); BAD(572); FGF3(2248); |
| | | PAK1(5058); FZD4(8322); SESN3(143686); CASP5(838); PPP2R1B(5519); |
| | | SCN4B(6330); BCL9L(283149); TNFRSF7(939); APOLD1(81575); TEGT |
| | | (7009); ESPL1(9700); CDK2(1017); CDCA3(83461); HDAC7A(51564); |
| | | CDK4(1019); PAWR(5074); DIP13B(55198); CDK2AP1(8099); HSMPP8(54737); |
| | | FGF9(2254); TNFRSF19(55504); BRCA2(675); RB1(5925); TFDP1 |
| | | (7027); CDC16(8881); BCL2L2(599); KIAA0323(23351); TXNDC(81542); PSEN1 |
| | | (5663); C14orf111(51077); EML1(2009); LTBP2(4053); NEK9(91754); |
| | | PPP1R13B(23368); AKT1(207); BUB1B(701); TMOD3(29766); MAPK6 |
| | | (5597); CSK(1445); DAPK2(23604); CSNK1G1(53944); KREMEN2(79412); |
| | | DNASE1(1773); CYLD(1540); RBL2(5934); CDT1(81620); GSPT1(2935); |
| | | MAPK3(5595); CSNK2A2(1459); SLC12A4(6560); PAFAH1B1(5048); GSG2 |
| | | (83903); TNFSF12(8742); ALOX15B(247); TRAF4(9618); LIG3(3980); |
| | | MAPT(4137); GOSR2(9570); NGFR(4804); TLK2(11011); NALP1(22861); |
| | | TP53(7157); GAS7(8522); BECN1(8678); BRCA1(672); HDAC5(10014); PHB |
| | | (5245); MALT1(10892); TXNL1(9352); GADD45B(4616); CHAF1A(10036); |
| | | UHRF1(29128); PIK3R2(5296); ZNF431(170959); ARHGEF1(9138); |
| | | APOE(348); FOSB(2354); BCL2L12(83596); AURKC(6795); HKR2(342945); |
| | | TNFSF14(8740); DPF1(8193); POFUT1(23509); STK4(6789); CD40(958); |
| | | RBL1(5933); DIDO1(11083); SEPT5(5413); ADORA2A(135); GAS2L1 |
| | | (10634); NF2(4771); MTP18(51537); MAPK1(5594); DMC1(11144); PDGFB |
| | | (5155); HDAC6(10013); TAF1(6872); NGFRAP1(27018); PPP2R3B(28227); |
| | | SMC1L1(8243); OPHN1(4983); BTK(695); SEPT6(23157); PDCD8(9131); |
| | | FAM58A(92002); HCFC1(3054); |
| hsa-miR-455 | 94; 784; 785; | PRKCZ(5590); KIF1B(23095); NUDC(10726); RBBP4(5928); MACF1(23499); |
| | 786; 787; | SH3GLB1(51100); CDC7(8317); BCL9(607); UHMK1(127933); IL19(29949); |
| | 788; 789; 790; | ZBTB17(7709); NOTCH2(4853); ASPM(259266); PPP1CB(5500); HK2 |
| | 2256; | (3099); PROC(5624); ZAK(51776); SEPT2(4735); BOK(666); CARD 12(58484); |
| | | TGFA(7039); FANCD2(2177); MCM2(4171); KNG1(3827); GSK3B |
| | | (2932); STAG1(10274); PIK3CB(5291); TNFSF10(8743); HD(3064); FGF5 |
| | | (2250); GAK(2580); APBB2(323); CCNI(10983); ERBB2IP(55914); UNC5A |
| | | (90249); PRLR(5618); BIRC1(4671); PJA2(9867); SEMA6A(57556); IRF1 |
| | | (3659); E2F3(1871); LTA(4049); PARC(23113); SASH1(23328); MDC1(965 |

Table 4-59

| | | |
|---|---|---|
| | | 6); IHPK3(117283); CUL7(9820); TNFRSF21(27242); DST(667); PLAGL1 |
| | | (5325); LPAL2(80350); EGFR(1956); ADAM22(53616); CASP2(835); SFRP4 |
| | | (6424); POLR3D(661); TRIM35(23087); MOS(4342); TP53INP1(94241); |
| | | TNFRSF11B(4982); MTSS1(9788); RECK(8434); CDK9(1025); BAG1(573); |
| | | SEMA4D(10507); CIZ1(25792); ZMYND11(10771); UNC5B(219699); PTEN |
| | | (5728); BTRC(8945); DOCK1(1793); PARD3(56288); C10orf72(196740); |
| | | C10orf46(143384); RPS6KB2(6199); LRP5(4041); BIRC3(330); APBB1 |
| | | (322); CLCF1(23529); PAK1(5058); HBXAP(51773); SESN3(143686); H2AFX |
| | | (3014); C11orf38(399967); LTBR(4055); TNFRSF7(939); PHC1(1911); |
| | | APAF1(317); BCAT1(586); MCRS1(10445); DDIT3(1649); ANAPC5(51433); |
| | | RFP2(10206); LATS2(26524); BCL2L2(599); TMEM63C(57156); KIAA0125 |
| | | (9834); CHES1(1112); AKT1(207); JAG2(3714); IL16(3603); SLC12A6 |
| | | (9990); CYLD(1540); E2F4(1874); CTCF(10664); PKMYT1(9088); GSPT1 |
| | | (2935); SIAH1(6477); TXNL4B(54957); ALOX15B(247); NLK(51701); CROP |
| | | (51747); BIRC5(332); CARD14(79092); MNT(4335); GPS2(2874); GAS2L2 |
| | | (246176); SOCS3(9021); C17orf62(79415); CIDEA(1149); DNM2(1785); |
| | | CARD8(22900); MCM8(84515); PARD6B(84612); C20orf11(54994); UBE2V1 |
| | | (7335); SYCP2(10388); DIDO1(11083); BTG3(10950); GTSE1(51512); |
| | | ADM2(79924); MN1(4330); BIRC4(331); CD40LG(959); DKC1(1736); SPIN2 |
| | | (54466); ARHGEF6(9459); |
| hsa-miR-483 | 95; 791; 2257; | TNFRSF1B(7133); SFN(2810); MACF1(23499); CDC20(991); SH3GLB1 |
| | | (51100); PROK1(84432); BNIPL(149428); PEA15(8682); UHMK1(127933); |
| | | MDM4(4194); IL24(11009); C1orf69(200205); CIAS1(114548); TNFRSF18 |
| | | (8784); TNFRSF9(3604); FRAP1(2475); ZBTB17(7709); E2F2(1870); YARS |
| | | (8565); MUTYH(4595); NOTCH2(4853); PYGO2(90780); TNFSF4(7292); |
| | | BIRC6(57448); HTRA2(27429); KCNIP3(30818); ZAK(51776); CASP10 |
| | | (843); CARD12(58484); EDAR(10913); ANAPC1(64682); ERCC3(2071); MCM6 |
| | | (4175); CLK1(1195); HDAC4(9759); KIF1A(547); FANCD2(2177); MCM2 |
| | | (4171); PSMD2(5708); TP73L(8626); OPA1(4976); ZNF445(353274); |
| | | C3orf62(375341); RASSF1(11186); TMEM110(375346); PROK2(60675); |
| | | GSK3B(2932); TNFSF10(8743); WHSC1(7468); HD(3064); IL8(3576); AFF1 |
| | | (4299); FGF2(2247); PLK4(10733); LETM1(3954); CCNI(10983); DKK2 |
| | | (27123); SFRP2(6423); PDCD6(10016); APC(324); CSNK1G3(1456); UNC5A |
| | | (90249); ZNF346(23567); BRD9(65980); PRKAA1(5562); ATG12(9140); |
| | | IRF1(3659); NRG2(9542); PFDN1(5201); HDAC3(8841); LY86(9450); E2F3 |
| | | (1871); AIF1(199); FOXO3A(2309); MDC1(9656); BAK1(578); CUL7(9820); |
| | | DST(667); BCLAF1(9774); PLAGL1(5325); LATS1(9113); AHR(196); |
| | | LOC441257(441257); RBM33(155435); TRIAD3(54476); SOSTDC1(25928); |
| | | CARD4(10392); SFRP4(6424); INHBA(3624); WBSCR18(84277); CDK6 |
| | | (1021); MCM7(4176); ZC3HC1(51530); TNFRSF10C(8794); PTK2B(2185); |
| | | TACC1(6867); UBE2V2(7336); FZD6(8323); WISP1(8840); LZTS1(11178); |
| | | TNFRSF10B(8795); TNFRSF10D(8793); CLU(1191); DKK4(27121); |
| | | TNFRSF11B(4982); LYNX1(66004); ANKRD15(23189); CKS2(1164); ZDHHC21 |
| | | (340481); CDKN2A(1029); CDKN2B(1030); BAG1(573); PTCH(5727); |
| | | CTNNAL1(8727); CIZ1(25792); CARD9(64170); ANAPC2(29882); DKK1 |
| | | (22943); UNC5B(219699); FAS(355); KIF11(3832); BTRC(8945); SUFU |
| | | (51684); PDCD11(22984); BAG3(9531); DOCK1(1793); PARD3(56288); KCNMA1 |
| | | (3778); DLG5(9231); MKI67(4288); LGALS12(85329); FADD(8772); |
| | | ATM(472); PKNOX2(63876); EI24(9538); IGF2(3481); CCND2(894); TNFRSF7 |
| | | (939); CNAP1(9918); PTPN6(5777); KLHDC5(57542); ESPL1(9700); |
| | | ING4(51147); BCAT1(586); HDAC7A(51564); MCRS1(10445); FMNL3 |
| | | (91010); PAWR(5074); BTG1(694); PPP1CC(5501); DIABLO(56616); FGF9 |
| | | (2254); ATP8A2(51761); BRCA2(675); RB1(5925); ING1(3621); NEK3 |
| | | (4752); LIG4(3981); BCL2L2(599); VASH1(22846); TRAF3(7187); NFKBIA |
| | | (4792); CDKL1(8814); LTBP2(4053); NEK9(91754); CHES1(1112); AKT1 |
| | | (207); JAG2(3714); IGF1R(3480); DAPK2(23604); CSNK1G1(53944); PDCD7 |
| | | (10081); TRAF7(84231); CCNF(899); DNAJA3(9093); RBL2(5934); KATNB1 |
| | | (10300); NOL3(8996); ELMO3(79767); CDT1(81620); CDK10(8558); |
| | | NME3(4832); ERN2(10595); DNAJA2(10294); SLC12A4(6560); PAFAH1B1 |
| | | (5048); MAPK7(5598); NF1(4763); TADA2L(6871); NGFR(4804); PRKCA |
| | | (5578); MAP2K6(5608); ANAPC11(51529); GAS7(8522); SPAG5(10615); |
| | | TIAF1(9220); GAS2L2(246176); BRCA1(672); ACOX1(51); CIDEA(1149); |
| | | DCC(1630); FZR1(51343); HMG20B(10362); CHAF1A(10036); DNM2 |
| | | (1785); MAP3K10(4294); ZNF226(7769); APOE(348); GRLF1(2909); CRX |
| | | (1406); PPP2R1A(5518); TNFSF14(8740); DPF1(8193); LOC284323(2843 |

Table 4-60

| | | |
|---|---|---|
| | | 23); LIG1(3978); CARD8(22900); KLK10(5655); CEP250(11190); STK4(6789); |
| | | CD40(958); ZHX3(23051); CABLES2(81928); DIDO1(11083); SON(6651); |
| | | BTG3(10950); ITGB2(3689); SMARCB1(6598); ADORA2A(135); GAS2L1 |
| | | (10634); MTP18(51537); MCM5(4174); BIK(638); MAPK8IP2(23542); |
| | | BID(637); SSTR3(6753); TOB2(10766); NHP2L1(4809); TAF1(6872); AGTR2 |
| | | (186); BIRC4(331); STAG2(10735); RPL10(6134); SMC1L1(8243); HCFC1 |
| | | (3054); |
| hsa-miR-485-5p | 96; 2258; 2259; | SAMD11(148398); TP73(7161); DFFB(1677); H6PD(9563); TNFRSF1B(7133); |
| | | NBL1(4681); KHDRBS1(10657); LCK(3932); RBBP4(5928); MACF1 |
| | | (23499); NFYC(4802); CDC20(991); PLK3(1263); CDKN2C(1031); RAP1A |
| | | (5906); CD2(914); BCL9(607); BNIPL(149428); CKS1B(1163); PEA15(8682); |
| | | UHMK1(127933); FASLG(356); MDM4(4194); IL24(11009); TRAF5(7188); |
| | | C1orf69(200205); CIAS1(114548); CDC2L2(985); TNFRSF9(3604); DFFA |
| | | (1676); FRAP1(2475); MAD2L2(10459); RCC2(55920); E2F2(1870); YARS |
| | | (8565); CLSPN(63967); MUTYH(4595); TESK2(10420); BCL10(8915); |
| | | GFI1(2672); CLCC1(23155); NOTCH2(4853); PYGO2(90780); DEDD(9191); |
| | | ASPM(259266); TP53BP2(7159); RHOB(388); MAPRE3(22924); PPP1CB |
| | | (5500); BIRC6(57448); PRKCE(5581); MSH2(4436); CCT7(10574); HTRA2 |
| | | (27429); HK2(3099); TCF7L1(83439); KCNIP3(30818); BRRN1(23397); |
| | | MRPL30(51263); RIF1(55183); CFLAR(8837); CASP10(843); CASP8 |
| | | (841); FZD7(8324); INHA(3623); CGREF1(10669); PPM1G(5496); EIF2AK2 |
| | | (5610); CCT4(10575); EDAR(10913); ANAPC1(64682); BIN1(274); PAX3 |
| | | (5077); CUL3(8452); HDAC4(9759); FANCD2(2177); VHL(7428); PCAF(8850); |
| | | MLH1(4292); TDGF1(6997); APPL(26060); ARGFX(503582); DTX3L |
| | | (151636); FAIM(55179); RNF7(9616); OPA1(4976); TADA3L(10474); RAF1 |
| | | (5894); TRAIP(10293); NEK4(6787); ATR(545); WHSC1(7468); HD(3064); |
| | | CD38(952); EREG(2069); SEPT11(55752); CCNG2(901); FGF5(2250); |
| | | NFKB1(4790); TLR2(7097); GAK(2580); LEF1(51176); CASP6(839); ANAPC10 |
| | | (10393); SKP2(6502); NIPBL(25836); CARD6(84674); ERBB2IP(55914); |
| | | RAD17(5884); F2R(2149); APC(324); RAD50(10111); TCF7(6932); BNIP1 |
| | | (662); UNC5A(90249); DAP(1611); PRLR(5618); UNG2(10309); BIRC1 |
| | | (4671); F2RL2(2151); MCC(4163); ATG12(9140); SEMA6A(57556); IRF1 |
| | | (3659); PPP2CA(5515); NRG2(9542); CSNK1A1(1452); CD74(972); FBXW11 |
| | | (23291); DDX41(51428); RNF130(55819); E2F3(1871); GMNN(51053); |
| | | TCF19(6941); TNF(7124); MAPK13(5603); CDKN1A(1026); TTBK1(84630); |
| | | IL17(3605); FOXO3A(2309); TNFAIP3(7128); HECA(51696); SASH1 |
| | | (23328); NEDD9(4739); DHX16(8449); MDC1(9656); DAXX(1616); TREML2 |
| | | (79865); CUL7(9820); TNFRSF21(27242); DST(667); LATS1(9113); PDCD2 |
| | | (5134); FOXK1(221937); AHR(196); SEPT7(989); EGFR(1956); FZD1 |
| | | (8321); STAG3(10734); RINT1(60561); BCAP29(55973); CASP2(835); PMS2 |
| | | (5395); CYCS(54205); CARD4(10392); ELMO1(9844); IGFBP3(3486); LOC401357 |
| | | (401357); YWHAG(7532); CDK6(1021); HIPK2(28996); FASTK |
| | | (10922); TNFRSF10C(8794); PTK2B(2185); FZD3(7976); UNC5D(137970); |
| | | BAG4(9530); TACC1(6867); SULF1(23213); TERF1(7013); E2F5(1875); |
| | | FZD6(8323); EBAG9(9166); MYC(4609); WISP1(8840); GML(2765); ZFP41 |
| | | (286128); LZTS1(11178); BIN3(55909); TNFRSF10B(8795); TNFRSF10D |
| | | (8793); ADRA1A(148); TRIM35(23087); CLU(1191); SFRP1(6422); DKK4 |
| | | (27121); CCNE2(9134); TP53INP1(94241); MTSS1(9788); ST3GAL1 |
| | | (6482); ANKRD15(23189); UNC13B(10497); DAPK1(1612); C9orf4(286223); |
| | | INVS(27130); ABL1(25); IFNW1(3467); CDKN2A(1029); TAF1L(138474); |
| | | FANCG(2189); SEMA4D(10507); PTCH(5727); AKNA(80709); FBXW2 |
| | | (26190); TRAF1(7185); CIZ1(25792); TSC1(7248); CARD9(64170); SGPL1 |
| | | (8879); UNC5B(219699); TSPAN14(81619); PTEN(5728); FRAT1(10023); |
| | | LZTS2(84445); BTRC(8945); NOLC1(9221); SUFU(51684); PDCD11 |
| | | (22984); TCF7L2(6934); BAG3(9531); PARD3(56288); DLG5(9231); SFRP5 |
| | | (6425); FBXW4(6468); DCLRE1A(9937); TIAL1(7073); MXI67(4288); BNIP3 |
| | | (664); WEE1(7465); HIPK3(10114); MDK(4192); F2(2147); INCENP(3619); |
| | | LGALS12(85329); DPF2(5977); SSSCA1(10534); SIPA1(6494); LRP5 |
| | | (4041); LRRC51(220074); CUL5(8065); ATM(472); CD3E(916); IGF2(3481); |
| | | CDKN1C(1028); APBB1(322); ZBTB3(79842); HRASLS3(11145); BAD(572); |
| | | CDCA5(113130); MAP3K11(4296); BRMS1(25855); CLCF1(23529); PPP1CA |
| | | (5499); FGF4(2249); FGF3(2248); PAK1(5058); FZD4(8322); BCL9L |
| | | (283149); NTF3(4908); LTBR(4055); CNAP1(9918); PTMS(5763); PTPN6 |
| | | (5777); BCL2L14(79370); CDKN1B(1027); TEGT(7009); ESPL1(9700); PDE1B |
| | | (5153); RBMS2(5939); MDM2(4193); APAF1(317); GAS2L3(283431 |

Table 4-61

| | | |
|---|---|---|
| | | ); RAN(5901); TNFRSF1A(7132); NOL1(4839); CDCA3(83461); HDAC7A |
| | | (51564); FAIM42(23017); C12orf22(81566); CBX5(23468); DDIT3(1649); WIF1 |
| | | (11197); PCTK2(5128); ANAPC5(51433); MPHOSPH9(10198); CDK2AP1 |
| | | (8099); CHFR(55743); HSMPP8(54737); TNFRSF19(55504); CDK8(1024); |
| | | BRCA2(675); RFP2(10206); FLJ40296(122183); ING1(3621); LATS2 |
| | | (26524); CSNK1A1L(122011); FOXO1A(2308); LIG4(3981); BCL2L2(599); |
| | | PNN(5411); TRAF3(7187); DAD1(1603); LTBP2(4053); NEK9(91754); TGFB3 |
| | | (7043); MOAP1(64112); PPP1R13B(23368); AKT1(207); JAG2(3714); |
| | | EIF3S1(8669); KIF23(9493); CYP1A2(1544); CSK(1445); SCAND2(54581); |
| | | IGF1R(3480); NDN(4692); BMF(90427); BNIP2(663); DAPK2(23604); CSNK1G1 |
| | | (53944); PDCD7(10081); BCL2A1(597); AP3S2(10239); KIAA1924 |
| | | (197335); TSC2(7249); CCNF(899); DNASE1(1773); DNAJA3(9093); CIITA |
| | | (4261); PLK1(5347); SPN(6693); CARD15(64127); CYLD(1540); RBL2 |
| | | (5934); CCL22(6367); E2F4(1874); PARD6A(50855); PDPR(55066); KIAA0513 |
| | | (9764); CDT1(81620); CDK10(8558); MGC13114(84326); NME3(4832); |
| | | PKMYT1(9088); GSPT1(2935); ERN2(10595); FLJ45256(400511); SEPT1 |
| | | (1731); PYCARD(29108); TRADD(8717); SLC12A4(6560); SMPD3(55512); |
| | | TERF2(7014); EXOSC6(118460); TXNL4B(54957); PCOLN3(5119); GSG2 |
| | | (83903); ALOX12(239); TNFSF12(8742); MAPK7(5598); NLK(51701); |
| | | NF1(4763); CDC6(990); MAPT(4137); NGFR(4804); PPM1D(8493); PRKCA |
| | | (5578); BIRC5(332); CARD14(79092); MNT(4335); TP53(7157); SPAG5 |
| | | (10615); TIAF1(9220); LOC201181(201181); BRCA1(672); HDAC5(10014); |
| | | GJA7(10052); ERN1(2081); SUMO2(6613); CSNK1D(1453); CABLES1 |
| | | (91768); MAPRE2(10982); MAPK4(5596); DCC(1630); MALT1(10892); PHLPP |
| | | (23239); BCL2(596); PARD6G(84552); CSNK1G2(1455); UHRF1(29128); |
| | | TNFSF9(8744); DNM2(1785); PIK3R2(5296); UBA52(7311); APLP1 |
| | | (333); PSMD8(5714); MAP3K10(4294); AXL(558); PVR(5817); BCL3(602); |
| | | APOE(348); CD3EAP(10849); FOSB(2354); HIF3A(64344); SPHK2(56848); |
| | | BAX(581); ATF5(22809); PPP2R1A(5518); LOC390980(390980); MOBKL2A |
| | | (126308); MATK(4145); DAPK3(1613); SIRT2(22933); ERCC2(2068); |
| | | LIG1(3978); CARD8(22900); CGB7(94027); KLK10(5655); A1BG(1); CDC25B |
| | | (994); MCM8(84515); PLCB1(23236); CEP250(11190); MYBL2(4605); |
| | | STK4(6789); PARD6B(84612); CSNK2A1(1457); ELMO2(63916); AURKA |
| | | (6790); DIDO1(11083); SON(6651); APP(351); ITGB2(3689); CDC45L |
| | | (8318); SEPT5(5413); ADORA2A(135); GAS2L1(10634); NF2(4771); YWHAH |
| | | (7533); MCM5(4174); EP300(2033); PPARA(5465); GTSE1(51512); MAPK8IP2 |
| | | (23542); BID(637); MAPK1(5594); PPM1F(9647); MN1(4330); CHEK2 |
| | | (11200); PDGFB(5155); TOB2(10766); PDHA1(5160); PCTK1(5127); |
| | | HDAC6(10013); TAF1(6872); CD40LG(959); TRAPPC2(6399); CUL4B(8450); |
| | | CETN2(1069); HCFC1(3054); |
| hsa-miR-488 | 97; 2260; | NBL1(4681); C1orf130(400746); HDAC1(3065); MACF1(23499); CDC7(8317); |
| | | CDC14A(8556); SYCP1(6847); FAM46C(54855); PEA15(8682); ADORA1 |
| | | (134); CTNNBIP1(56998); RCC2(55920); KIAA0319L(79932); C1orf163 |
| | | (65260); RBMXL1(494115); DBT(1629); DEDD(9191); ZNF512(84450); |
| | | BIRC6(57448); PRKCE(5581); MSH2(4436); ANKRD36(375248); BCL2L11 |
| | | (10018); RIF1(55183); CDCA7(83879); CFLAR(8837); YWHAQ(10971); |
| | | CARD12(58484); RTN4(57142); BUB1(699); WDR83(55339); FANCD2(2177); |
| | | MOBP(4336); GPR62(118442); GNL3(26354); NEK11(79858); SENP2 |
| | | (59343); TP73L(8626); TADA3L(10474); RAF1(5894); THRB(7068); KIAA1143 |
| | | (57456); AER61(285203); PROK2(60675); VGLL3(389136); LRRC15 |
| | | (131578); MOBKL1A(92597); EREG(2069); RPL34(6164); PLK4(10733); GAK |
| | | (2580); USP46(64854); SFRP2(6423); NEK1(4750); CASP3(836); CPEB4 |
| | | (80315); ATG12(9140); SPOCK1(6695); CDC23(8697); CDC25C(995); |
| | | HDAC8(8841); DDX41(51428); RNF130(55819); CD83(9308); TNF(7124); |
| | | C2(717); RNF8(9025); CDC5L(988); SLC35A1(10559); HECA(51696); GPR126 |
| | | (57211); ESR1(2099); IHPK3(117283); SUPT3H(8464); DSCR1L1(10231); |
| | | DST(667); CNR1(1268); FRK(2444); MAP3K5(4217); LATS1(9113); |
| | | PDCD2(5134); HDAC9(9734); CHN2(1124); SEPT7(989); DKFZp761I2123 |
| | | (83637); BCAP29(55973); ZNF398(57541); DRCTNNB1A(84668); IGF2BP3 |
| | | (10643); ELMO1(9844); CDK6(1021); BRAF(673); CHMP7(91782); ESCO2 |
| | | (157570); UNC5D(137970); TACC1(6867); RIPK2(8767); ZHX2(22882); |
| | | MYC(4609); BIN3(55909); RDHE2(195814); PAG1(55824); CCNE2(9134); |
| | | EXT1(2131); ANKRD15(23189); MGC21881(389741); NTRK2(4915); |
| | | CKS2(1164); FCMD(2218); LOC389791(389791); ABL1(25); CDKN2B(1030); |
| | | PPP3R2(5535); CTNNAL1(8727); ZMYND11(10771); SGPL1(8879); |

Table 4-62

| | | |
|---|---|---|
| | | KIF11(3832); SUFU(51684); PDCD11(22984); CASP7(840); GPR26(2849); |
| | | SMNDC1(10285); GPAM(57678); MKI67(4288); GAS2(2620); STX3A(6809); |
| | | ATM(472); DIXDC1(85458); CD3G(917); ARHGEF12(23365); SLC1A2 |
| | | (6506); BRMS1(25855); FZD4(8322); SESN3(143686); CCND2(894); CLEC2D |
| | | (29121); FGD4(121512); PA2G4(5036); MDM2(4193); APAF1(317); |
| | | GAS2L3(283431); BRI3BP(140707); ING4(51147); LRP6(4040); KRAS(3845); |
| | | C12orf22(81566); LOC144501(144501); CDK4(1019); DUSP6(1848); |
| | | BRCA2(675); CSNK1A1L(122011); NRXN3(9369); FLJ11806(79882); ACIN1 |
| | | (22985); STRN3(29966); NEK9(91754); C14orf143(90141); MOAP1(64112); |
| | | BAG5(9529); MAPK6(5597); PLEKHQ1(80301); CSNK1G1(53944); |
| | | MTFMT(123263); KREMEN2(79412); PRKCB1(5579); CYLD(1540); RBL2 |
| | | (5934); CYB5B(80777); AXIN1(8312); GSPT1(2935); ARL6IP(23204); P8 |
| | | (26471); SIAH1(6477); BIRC4BP(54739); WSB1(26118); NF1(4763); KCNJ2 |
| | | (3759); CDK3(1018); GPS1(2873); TBC1D3C(414060); BECN1(8678); |
| | | MPO(4353); SEPT4(5414); USP36(57602); CBX4(8535); CIDEA(1149); RIOK3 |
| | | (8780); SETBP1(26040); MAPK4(5596); PHLPP(23239); SERPINB2 |
| | | (5055); BCL2(596); TXNDC10(54495); FZR1(51343); LOC284434(284434); |
| | | CEBPG(1054); FLJ40235(284369); C19orf31(404664); ZNF562(54811); |
| | | DNASE2(1777); DEDD2(162989); CARD8(22900); CTNNBL1(56259); NCOA3 |
| | | (8202); ARFGEF2(10564); CSE1L(1434); CTCFL(140690); SYCP2 |
| | | (10388); DIDO1(11083); USP16(10600); ETS2(2114); SEPT5(5413); MAPK1 |
| | | (5594); LOC400927(400927); CBX7(23492); CLCN4(1183); AGTR2(186); |
| | | BIRC4(331); AFF2(2334); ARHGAP6(395); RPGR(6103); SPIN2(54466); ZNF75 |
| | | (7626); ARHGEF6(9459); |
| hsa-miR-489 | 98; 792; 793; | TNFRSF1B(7133); RCC1(1104); RBBP4(5928); PROK1(84432); RBM8A |
| | 794; 795; | (9939); BCL9(607); UHMK1(127933); IL19(29949); IL24(11009); TNFRSF25 |
| | 796; | (8718); DFFA(1676); FRAP1(2475); FAF1(11124); S100A6(6277); ASPM |
| | | (259266); PPP1CB(5500); BIRC6(57448); MSH2(4436); KCNIP3(30818); |
| | | PROC(5624); RIF1(55183); PMS1(5378); ALS2CR19(117583); PSMD1(5707); |
| | | RTN4(57142); EDAR(10913); ANAPC1(64682); FRZB(2487); STAT1 |
| | | (6772); HDAC4(9759); PDCD6IP(10015); RAF1(5894); NEK4(6787); DNASE1L3 |
| | | (1776); UBE1C(9039); PROK2(60675); DCBLD2(131566); PTPLB |
| | | (201562); TFDP2(7029); HCAP-G(64151); SPP1(6696); PLK4(10733); TLR2 |
| | | (7097); DKK2(27123); PDCD6(10016); CARD6(84674); RAD17(5884); F2R |
| | | (2149); GDNF(2668); SEMA6A(57556); DUSP1(1843); PARC(23113); CDC5L |
| | | (988); ANKRD6(22881); FOXO3A(2309); CCND3(896); CUL7(9820); |
| | | DST(667); ATG5(9474); SGK(6446); BCLAF1(9774); PDCD2(5134); STK17A |
| | | (9263); FZD9(8326); CASP2(835); PMS2(5395); SOSTDC1(25928); ELMO1 |
| | | (9844); SFRP4(6424); INHBA(3624); PTN(5764); FASTK(10922); FZD3 |
| | | (7976); UNC5D(137970); TACC1(6867); UBE2V2(7336); RIPK2(8767); |
| | | TNFRSF10B(8795); CLU(1191); ANKRD15(23189); UNC13B(10497); RECK |
| | | (8434); TAF1L(138474); PTCH(5727); TRAF1(7185); TSC1(7248); SGPL1 |
| | | (8879); TCF7L2(6934); CPEB3(22849); MKI67(4288); F2(2147); INCENP |
| | | (3619); LGALS12(85329); ATM(472); EI24(9538); FZD4(8322); ZW10(9183); |
| | | LTBR(4055); TEGT(7009); PA2G4(5036); MDM2(4193); RAN(5901); |
| | | BCAT1(586); BTG1(694); MPHOSPH9(10198); CHFR(55743); BRCA2(675); |
| | | ING1(3621); CDC16(8881); LATS2(26524); KIAA1008(22894); LIG4(3981); |
| | | TXNDC(81542); TRAF3(7187); GZMH(2999); GZMB(3002); EGLN3 |
| | | (112399); PGF(5228); TGFB3(7043); GTF2A1(2957); MOAP1(64112); MAPK6 |
| | | (5597); TLN2(83660); IGF1R(3480); BMF(90427); CCNF(899); KATNB1 |
| | | (10300); PKMYT1(9088); ERN2(10595); FTS(64400); APPBP1(8883); SLC12A4 |
| | | (6560); TXNL4B(54957); ALOX15B(247); NLK(51701); PRKCA(5578); |
| | | CARD14(79092); P2RX1(5023); BRCA1(672); MPO(4353); CABLES1 |
| | | (91768); MAPRE2(10982); BCL2(596); FZR1(51343); ATF5(22809); NALP2 |
| | | (55655); ZIK1(284307); TGFB1(7040); DEDD2(162989); CARD8(22900); |
| | | CD40(958); RBL1(5933); SYCP2(10388); USP16(10600); SON(6651); ITGB2 |
| | | (3689); MCM5(4174); MAPK1(5594); CARD10(29775); TAF1(6872); STAG2 |
| | | (10735); ATP2B3(492); FAM9C(171484); BTK(695); CUL4B(8450); PDCD8 |
| | | (9131); ARHGEF6(9459); HCFC1(3054); |
| hsa-miR-491 | 99; 797; | VWA1(64856); PRDM16(63976); TNFRSF1B(7133); NBL1(4681); TCEB3 |
| | | (6924); SESN2(83667); KHDRBS1(10657); LCK(3932); MACF1(23499); SLC6A17 |
| | | (388662); PROK1(84432); BCL9(607); PEA15(8682); MDM4(4194); |
| | | CDC2L2(985); TNFRSF14(8764); GPR153(387509); FRAP1(2475); E2F2 |
| | | (1870); TESK2(10420); NOTCH2(4853); MCL1(4170); GATAD2B(57459); |
| | | DEDD(9191); TNNI1(7135); CDC42BPA(8476); RHOB(388); MAPRE3 |

Table 4-63

| | | |
|---|---|---|
| | | (22924); BIRC6(57448); PRKCE(5581); HTRA2(27429); BRRN1(23397); BCL2L11 |
| | | (10018); RIF1(55183); TAIP-2(80034); ZAK(51776); CASP10(843); |
| | | FZD7(8324); INHA(3623); COL4A3(1285); PSMD1(5707); BOK(666); CGREF1 |
| | | (10669); PPM1G(5496); TGFA(7039); SEPT10(151011); OGG1(4968); |
| | | MCM2(4171); SENP2(59343); TP73L(8626); SRGAP3(9901); CIDEC(63924); |
| | | RAF1(5894); DNASE1L3(1776); ATR(545); HD(3064); IL8(3576); SPP1 |
| | | (6696); GAK(2580); DKK2(27123); LEF1(51176); NEK1(4750); MRPS30 |
| | | (10884); F2R(2149); CSNK1G3(1456); RAD50(10111); TCF7(6932); UNC5A |
| | | (90249); FGF10(2255); SEMA6A(57556); NME5(8382); CDC23(8697); |
| | | NRG2(9542); CD14(929); DDX41(51428); RNF130(55819); LTA(4049); AIF1 |
| | | (199); CSNK2B(1460); SYNGAP1(8831); CDKN1A(1026); FOXP4(116113); |
| | | PARC(23113); VEGF(7422); FOXO3A(2309); HECA(51696); NEDD9 |
| | | (4739); MDC1(9656); DAXX(1616); IHPK3(117283); CCND3(896); TNFRSF21 |
| | | (27242); MCM3(4172); DST(667); ATG5(9474); SESN1(27244); FRK(2444); |
| | | MAP3K5(4217); PLAGL1(5325); EGFR(1956); FZD9(8326); ELN(2006); |
| | | LOC441257(441257); MAD1L1(8379); TRIAD3(54476); CDK6(1021); |
| | | HIPK2(28996); FASTK(10922); POLR3D(661); TNFRSF10C(8794); PTK2B |
| | | (2185); UNC5D(137970); ZNF703(80139); BAG4(9530); TACC1(6867); |
| | | TERF1(7013); E2F5(1875); WISP1(8840); BIN3(55909); TNFRSF10B(8795); |
| | | TRIM35(23087); RAD21(5885); MTSS1(9788); ANKRD15(23189); C9orf47 |
| | | (286223); INVS(27130); CDK9(1025); ABL1(25); CDKN2B(1030); MOBKL2B |
| | | (79817); FANCG(2189); SEMA4D(10507); TNFSF8(944); PPP6C |
| | | (5537); CIZ1 (25792); TSC1(7248); C10orf9(219771); UNC5B(219699); FRAT1 |
| | | (10023); LZTS2(84445); BTRC(8945); SUFU(51684); PDCD11(22984); |
| | | BAG3(9531); DOCK1(1793); DLG5(9231); SFRP5(6425); FGF8(2253); |
| | | SH3PXD2A(9644); C10orf46(143384); TIAL1(7073); MKI67(4288); STIM1 |
| | | (6786); HIPK3(10114); MDK(4192); INCENP(3619); VEGFB(7423); DPF2 |
| | | (5977); SSSCA1(10534); SIPA1(6494); IGF2(3481); RHOG(391); EIF4G2 |
| | | (1982); DKK3(27122); CDCA5(113130); MAP3K11(4296); CLCF1(23529); |
| | | FGF3(2248); PPP2R1B(5519); BCL9L(283149); CNAP1(9918); PDE1B(5153); |
| | | APAF1(317); ING4(51147); BCAT1(586); KRAS(3845); FAIM2(23017); |
| | | C12orf22(81566); CDK4(1019); ANAPC7(51434); HRK(8739); DIABLO |
| | | (56616); SCARB1(949); CHFR(55743); ATP11A(23250); TFDP1(7027); CSNK1A1L |
| | | (122011); BCL2L2(599); TRAF3(7187); ACIN1(22985); CIDEB(27141); |
| | | SPTB(6710); CHES1(1112); PRIMA1(145270); PPP1R13B(23368); |
| | | CCNB2(9133); AVEN(57099); BMF(90427); CSNK1G1(53944); FLJ22795 |
| | | (80154); RAB40C(57799); TSC2(7249); TRAF7(84231); CCNF(899); DNASE1 |
| | | (1773); DNAJA3(9093); CARD15(64127); RBL2(5934); GNAO1(2775); |
| | | CTCF(10664); CDT1(81620); CDK10(8558); PKMYT1(9088); MAPK3(5595); |
| | | SMPD3(55512); TXNL4B(54957); PCOLN3(5119); HIC1(3090); GSG2 |
| | | (83903); NF1(4763); LIG3(3980); IGF2BP1(10642); NGFR(4804); PPM1D |
| | | (8493); PRKCA(5578); TMEM104(54868); CARD14(79092); ANAPC11(51529); |
| | | GPS1(2873); MNT(4335); NALP1(22861); TP53(7157); GAS7(8522); |
| | | SPAG5(10615); GIT1(28964); DUSP3(1845); HDAC5(10014); MPO(4353); |
| | | CSNK1D(1453); CIDEA(1149); CABLES1(91768); MAPK4(5596); DCC(1630); |
| | | FZR1(51343); CHAF1A(10036); TNFSF9(8744); PIN1(5300); PIK3R2 |
| | | (5296); NDUFA13(51079); CCNE1(898); APLP1(333); SAMD4B(55095); |
| | | MAP3K10(4294); AXL(558); ARHGEF1(9138); APOE(348); FOSB(2354); |
| | | BAX(581); BCL2L12(83596); ATF5(22809); SPIB(6689); NALP2(55655); |
| | | DAPK3(1613); TNFSF14(8740); ELAVL3(1995); TGFB1(7040); ERF(2077); |
| | | CARD8(22900); KLK10(5655); MCM8(84515); MYBL2(4605); STK4(6789); |
| | | CSE1L(1434); BCL2L1(598); E2F1(1869); ELMO2(63916); UBE2V1 |
| | | (7335); DIDO1(11083); PDXK(8566); APP(351); SNF1LK(150094); ITGB2 |
| | | (3689); BCL2L13(23786); ADORA2A(135); GAS2L1(10634); NF2(4771); MCM5 |
| | | (4174); EP300(2033); SEPT3(55964); GTSE1(51512); MAPK1(5594); |
| | | PPM1F(9647); MN1(4330); CARD10(29775); PDGFB(5155); TOB2(10766); |
| | | MAPK12(6300); CCNB3(85417); TAF1(6872); PPP2R3B(28227); FIGF |
| | | (2277); SMC1LI(8243); BTK(695); SEPT6(23157); FAM58A(92002); HCFC1 |
| | | (3054); |
| hsa-miR-493-5p | 100; | SESN2(83667); RBBP4(5928); MACF1(23499); SH3GLB1(51100); CDC14A |
| | | (8556); VANGL1(81839); UHMK1(127933); RASAL2(9462); TNFRSF9 |
| | | (3604); CTNNBIP1(56998); FRAP1(2475); ZBTB17(7709); RCC2(55920); |
| | | BCL10(8915); MCL1(4170); ASPM(259266); SEL1(85465); PPP1CB(5500); |
| | | BIRC6(57448); HK2(3099); BRRN1(23397); PDCL3(79031); TAIP-2(80034); |
| | | ZAK(51776); CASP10(843); FZD7(8324); CD28(940); NRP2(8828); E2 |

Table 4-64

| | | |
|---|---|---|
| | | F6(1876); PUM2(23369); EIF2AK2(5610); CDC42EP3(10602); SERTAD2 |
| | | (9792); TIA1(7072); AFF3(3899); IL1A(3552); ERCC3(2071); MCM6(4175); |
| | | IFIH1(64135); SLC25A12(8604); STAT1(6772); FZD5(7855); CUL3(8452); |
| | | VHL(7428); NR2C2(7182); APPL(26060); PCNP(57092); MCM2(4171); |
| | | PSMD2(5708); SENP2(59343); KNG1(3827); TP73L(8626); CAST1(26059); |
| | | VGLL3(389136); HD(3064); DKFZP686A01247(22998); IL8(3576); SEPT11 |
| | | (55752); CCNG2(901); FGF5(2250); FGF2(2247); PLK4(10733); ZNF721 |
| | | (170960); SCC-112(23244); GABRA2(2555); CLOCK(9575); UNC5C(8633); |
| | | DKK2(27123); ANAPC10(10393); NEK1(4750); VEGFC(7424); SKP2 |
| | | (6502); NIPBL(25836); CARD6(84674); CCNB1(891); TCF7(6932); UNC5A |
| | | (90249); BIRC1(4671); IRF1(3659); PPP2CA(5515); CSNK1A1(1452); FBXW11 |
| | | (23291); IBRDC2(255488); E2F3(1871); GMNN(51053); CDKN1A |
| | | (1026); FOXO3A(2309); HECA(51696); C6orf85(63027); SUPT3H(8464); |
| | | DST(667); MAP3K5(4217); HIVEP2(3097); AHR(196); SEPT7(989); STK17A |
| | | (9263); ADCY1(107); HBP1(26959); CUL1(8454); CYCS(54205); ELMO1 |
| | | (9844); SFRP4(6424); INHBA(3624); YWHAG(7532); TNFRSF10C(8794); |
| | | ESCO2(157570); UNC5D(137970); BAG4(9530); TACC1(6867); UBE2V2 |
| | | (7336); TERF1(7013); PSD3(23362); LZTS1(11178); TNFRSF10D(8793); |
| | | TNFRSF10A(8797); SFRP1(6422); TP53INP1(94241); LRP12(29967); TNFRSF11B |
| | | (4982); MTSS1(9788); ANKRD15(23189); CDC37L1(55664); CKS2 |
| | | (1164); GAS1(2619); PTCH(5727); TNFSF15(9966); TRAF1(7185); CARD9 |
| | | (64170); ZMYND11(10771); CUGBP2(10659); C10orf9(219771); PCGF5 |
| | | (84333); KIF11(3832); FRAT1(10023); SUFU(51684); CSPG6(9126); |
| | | TCF7L2(6934); FZD8(8325); ZWINT(11130); EGR2(1959); PPP3CB(5532); |
| | | FRAT2(23401); TIAL1(7073); PARVA(55742); GTF2H1(2965); API5(8539); |
| | | SIPA1(6494); CUL5(8065); ATM(472); IGF2(3481); WT1(7490); PPP1CA |
| | | (5499); FZD4(8322); PPP2R1B(5519); ZW10(9183); TRHDE(29953); GAS2L3 |
| | | (283431); ATP2A2(488); TNFRSF1A(7132); LRP6(4040); DDIT3(1649); |
| | | OSBPL8(114882); BTG1(694); EEA1(8411); PPP1CC(5501); SBNO1 |
| | | (55206); SCARB1(949); FGF9(2254); BRCA2(675); RFP2(10206); FLJ40296 |
| | | (122183); MBNL2(10150); ING1(3621); ATP11A(23250); CDC16(8881); |
| | | CSNK1A1L(122011); FOXO1A(2308); DACH1(1602); INSM2(84684); CDKN3 |
| | | (1033); VASH1(22846); NRXN3(9369); GPR65(8477); TRAF3(7187); |
| | | LTBP2(4053); BUB1B(701); KIF23(9493); SLC12A6(9990); BMF(90427); |
| | | TTBK2(146057); SCAMP2(10066); PDPK1(5170); DNAJA3(9093); ZNF720 |
| | | (124411); PAPD5(64282); CARD15(64127); CYLD(1540); RBL2(5934); |
| | | AXIN1(8312); GSPT1(2935); SMG1(23049); ERN2(10595); SLC12A4(6560); |
| | | MPHOSPH6(10200); ALOX12(239); NTN1(9423); MAPK7(5598); NLK |
| | | (51701); NF1(4763); RHOT1(55288); CROP(51747); PPM1D(8493); TLK2 |
| | | (11011); PRKCA(5578); SLC9A3R1(9368); TP53(7157); BRCA1(672); ANKRD12 |
| | | (23253); MIB1(57534); MAPK4(5596); DCC(1630); MALT1(10892); |
| | | TXNL1(9352); BCL2(596); CDC34(997); KLK2(3817); KLK10(5655); PLCB1 |
| | | (23236); STK4(6789); C20orf177(63939); RBL1(5933); ELMO2(63916); |
| | | ATP9A(10079); AURKA(6790); DIDO1(11083); USP16(10600); SNF1LK |
| | | (150094); BCL2L13(23786); PGEA1(25776); EP300(2033); SEPT3(55964); |
| | | MAPK1(5594); CHEK2(11200); TOB2(10766); BIRC4(331); RP6-213H19.1 |
| | | (51765); FIGF(2277); WNK3(65267); BTK(695); LAMP2(3920); |
| hsa-miR-494 | 101; 798; 799; | DFFB(1677); SFN(2810); NUDC(10726); KHDRBS1(10657); RBBP4(5928); |
| | | MACF1(23499); CD2(914); BCL9(607); CKS1B(1163); DAP3(7818); PEA15 |
| | | (8682); ATF6(22926); FASLG(356); PPP1R12B(4660); MDM4(4194); TRAF5 |
| | | (7188); TGFB2(7042); CCNL2(81669); FRAP1(2475); CLSPN(63967); |
| | | BCL10(8915); NOTCH2(4853); CD5L(922); B3GALT2(8707); ASPM(259266); |
| | | ZBTB41(360023); TP53BP2(7159); ENAH(55740); PPP1CB(5500); |
| | | BIRC6(57448); PRKCE(5581); SOCS5(9655); BRRN1(23397); PDCL3(79031); |
| | | RIF1(55183); LASS6(253782); PMS1(5378); CFLAR(8837); CASP10 |
| | | (843); BMPR2(659); CD28(940); EIF2AK2(5610); RTN4(57142); TIA1(7072); |
| | | BUB1(699); ANAPC1(64682); IL1A(3552); MCM6(4175); ARL5A(26225); |
| | | NCKAP1(10787); STAT1(6772); PGAP1(80055); CLK1(1195); BARD1 |
| | | (580); CUL3(8452); PCAF(8850); ZNF621(285268); APPL(26060); DZIP3(9666); |
| | | TP73L(8626); MAP4(4134); IL17RD(54756); UBE1C(9039); GSK3B |
| | | (2932); STAG1(10274); TNFSF10(8743); WHSC1(7468); HD(3064); HCAP-G |
| | | (64151); AFF1(4299); NFKB1(4790); FGF2(2247); LARP2(55132); MLR1 |
| | | (254251); BTC(685); CCNI(10983); FAM13A1(10144); ANKRD50(57182); |
| | | NEK1(4750); MFAP3L(9848); PDCD6(10016); NIPBL(25836); CARD6(84674); |
| | | ERBB2IP(55914); RAD17(5884); F2R(2149); SRFBP1(153443); PC |

Table 4-65

| | | |
|---|---|---|
| | | DHB9(56127); KCTD16(57528); PRLR(5618); MCC(4163); KIF3A(11127); |
| | | NME5(8382); FBXW11(23291); MYLIP(29116); TNF(7124); VEGF(7422); |
| | | PTP4A1(7803); MYO6(4646); TNFAIP3(7128); SASH1(23328); SYNCRIP |
| | | (10492); CNR1(1268); SESN1(27244); PDCD2(5134); HDAC9(9734); HBP1 |
| | | (26959); BCAP29(55973); CASP2(835); CUL1(8454); TRIAD3(54476); CDK6 |
| | | (1021); HIPK2(28996); BNIP3L(665); PTK2B(2185); ESCO2(157570); |
| | | UNC5D(137970); TACC1(6867); TERF1(7013); RDH10(157506); PI15(51050); |
| | | FZD6(8323); MYC(4609); TP53INP1(94241); STK3(6788); EXT1(2131); |
| | | VPS13A(23230); C9orf47(286223); INVS(27130); ABL1(25); IFNA2 |
| | | (3440); LRRC19(64922); BAG1(573); ZMYND11(10771); WDR37(22884); C10orf9 |
| | | (219771); SIRT1(23411); CXXC6(80312); SGPL1(8879); CHST3(9469); |
| | | PTEN(5728); KIF11(3832); TCF7L2(6934); DOCK1(1793); PARD3(56288); |
| | | CUL2(8453); ZWINT(11130); DLG5(9231); DCLRE1A(9937); C10orf118 |
| | | (55088); C10orf46(143384); BNIP3(664); API5(8539); EXT2(2132); STX3A |
| | | (6809); CCND1(595); C11orf30(56946); C11orf54(28970); BIRC3(330); |
| | | BIRC2(329); CUL5(8065); MLL(4297); WT1(7490); SLC1A2(6506); HRASLS3 |
| | | (11145); RBM4B(83759); PAK1(5058); FZD4(8322); CASP1(834); SCN2B |
| | | (6327); BCL9L(283149); CCND2(894); LTBR(4055); PTMS(5763); CDKN1B |
| | | (1027); TMTC3(160418); SOCS2(8835); GAS2L3(283431); KNTC1 |
| | | (9735); RAD52(5893); DUSP16(80824); BCAT1(586); HDAC7A(51564); LALBA |
| | | (3906); C12orf22(81566); DDIT3(1649); CDK4(1019); PAWR(5074); MPHOSPH9 |
| | | (10198); HSMPP8(54737); TNFRSF19(55504); BRCA2(675); RB1 |
| | | (5925); FLJ40296(122183); ING1(3621); CUL4A(8451); LATS2(26524); |
| | | FOXO1A(2308); KBTBD7(84078); LIG4(3981); C14orf92(9878); TXNDC |
| | | (81542); FBXO34(55030); PSEN1(5663); VASH1(22846); CDKL1(8814); DLG7 |
| | | (9787); CHES1(1112); MOAP1(64112); FGF7(2252); VPS13C(54832); |
| | | CSNK1G1(53944); ZNF694(342357); DNAJA2(10294); SLC12A4(6560); |
| | | H1C1(3090); PAFAH1B1(5048); TRAF4(9618); NF1(4763); UNC45B(146862); |
| | | CROP(51747); PPM1D(8493); TLK2(11011); BIRC5(332); CARD14(79092); |
| | | GAS7(8522); DSG2(1829); MAPK4(5596); PHLPP(23239); DOK6(220164); |
| | | SOCS6(9306); DLGAP1(9229); ESCO1(114799); TXNL1(9352); UHRF1 |
| | | (29128); CASP14(23581); APLP1(333); APOE(348); FOSB(2354); NALP2 |
| | | (55655); ZNF264(9422); AES(166); DAPK3(1613); BBC3(27113); PLCB1 |
| | | (23236); RBL1(5933); UBE2V1(7335); CTCFL(140690); SYCP2(10388); |
| | | DIDO1(11083); USP16(10600); SON(6651); CHAF1B(8208); MX1(4599); |
| | | BCL2L13(23786); ADORA2A(135); NF2(4771); GTSE1(51512); MAPK1(5594); |
| | | PPM1F(9647); MN1(4330); ZXDB(158586); FIGF(2277); ARHGEF6 |
| | | (9459); MTCP1(4515); |
| hsa-miR-501 | 102; 800; 2261; | PRKCZ(5590); NPAL3(57185); NUDC(10726); KHDRBS1(10657); MACF1 |
| | | (23499); DNAJC6(9829); SH3GLB1(51100); SYCP1(6847); RBM8A(9939); |
| | | CKS1B(1163); UHMK1(127933); C1orf21(81563); MDM4(4194); TRAF5 |
| | | (7188); ICMT(23463); CTNNBIP1(56998); CLSPN(63967); FAF1(11124); |
| | | NOTCH2(4853); PYGO2(90780); GLRX2(51022); ASPM(259266); ENAH |
| | | (55740); FH(2271); MAPRE3(22924); PPP1CB(5500); BIRC6(57448); PRKCE |
| | | (5581); ANKRD36(375248); RIF1(55183); ZAK(51776); CFLAR(8837); |
| | | CASP10(843); COL4A3(1285); CGREF1(10669); RTN4(57142); LOC51057 |
| | | (51057); TIA1(7072); ANAPC1(64682); IL1A(3552); ERCC3(2071); IFIH1 |
| | | (64135); CLK1(1195); ITGA9(3680); GNL3(26354); NEK11(79858); SMC4L1 |
| | | (10051); TP73L(8626); SLC4A7(9497); VGLL3(389136); MGC40579(256356); |
| | | ATR(545); CD38(952); LOC285429(285429); THAP6(152815); SEPT11 |
| | | (55752); FGF5(2250); PLK4(10733); CCNI(10983); DKK2(27123); LEF1 |
| | | (51176); NEK1(4750); CASP3(836); ERBB2IP(55914); DCP2(167227); |
| | | CSNK1G3(1456); GRAMD3(65983); SGCD(6444); PRLR(5618); MEF2C |
| | | (4208); ST8SIA4(7903); SEMA6A(57556); IBRDC2(255488); E2F3(1871); |
| | | FLJ45422(441140); CSNK2B(1460); BAG2(9532); PTP4A1(7803); TNFAIP3 |
| | | (7128); HECA(51696); QKI(9444); NEDD9(4739); DCDC2(51473); MDC1 |
| | | (9656); CCND3(896); CUL7(9820); SUPT3H(8464); MCM3(4172); DST |
| | | (667); CCNC(892); ATG5(9474); SGK(6446); BCLAF1(9774); PDCD2(5134); |
| | | AHR(196); HDAC9(9734); CUL1(8454); TRIAD3(54476); CYCS(54205); |
| | | CARD4(10392); INHBA(3624); KBTBD11(9920); MTMR9(66036); KIAA1456 |
| | | (57604); BAG4(9530); TACC1(6867); SULF1(23213); PI15(51050); RIPK2 |
| | | (8767); PINX1(54984); LZTS1(11178); SFRP1(6422); CCNE2 (9134); |
| | | MTSS1(9788); JAK2(3717); UNC13B(10497); RECK(8434); GADD45G(10912); |
| | | INVS(27130); ABL1(25); CDKN2B(1030); EDG2(1902); CIZ1(25792); |
| | | TSC1(7248); CARD9(64170); SIRT1(23411); C10orf42(90550); BTRC(8 |

Table 4-66

| | | |
|---|---|---|
| | | 945); CSPG6(9126); ADRB1(153); PARD3(56288); PPP3CB(5532); DCLRE1A |
| | | (9937); C10orf46(143384); MKI67(4288); DKFZp686O24166(374383); |
| | | GAS2(2620); HIPK3(10114); FADD(8772); BIRC2(329); CUL5(8065); ATM |
| | | (472); CD3G(917); STS-1(84959); LOH11CR2A(4013); CHEK1(1111); APBB1 |
| | | (322); EIF4G2(1982); MTL5(9633); FZD4(8322); SESN3(143686); CASP1 |
| | | (834); PPP2R1B(5519); KIAA0999(23387); BACE1(23621); BCL9L |
| | | (283149); H2AFX(3014); CCND2(894); LTBR(4055); RAN(5901); OACT5(10162); |
| | | LRP6(4040); BCAT1(586); C12orf22(81566); BTG1(694); PCTK2(5128); |
| | | DIP13B(55198); MPHOSPH9(10198); SCARB1(949); HSMPP8(54737); |
| | | RFP2(10206); FLJ40296(122183); CUL4A(8451); LATS2(26524); FLJ25477 |
| | | (219287); LIG4(3981); PNN(5411); KLHDC1(122773); CGRRF1(10668); |
| | | VASH1(22846); CDKL1(8814); LTBP2(4053); MOAP1(64112); BAG5 |
| | | (9529); FGF7(2252); KIAA1024(23251); SLC12A6(9990); CSNK1G1(53944); |
| | | KREMEN2(79412); DNASE1(1773); PAPD5(64282); CYLD(1540); NOL3 |
| | | (8996); GSPT1(2935); SLC7A6OS(84138); HIC1(3090); PAFAH1B1(5048); |
| | | ALOX15B(247); WSB1(26118); NLK(51701); MAPT(4137); NME1(4830); |
| | | TLK2(11011); SFRS2(6427); CSNK1D(1453); KIAA1328(57536); CCDC5 |
| | | (115106); RNF165(494470); DCC(1630); MALT1(10892); ROCK1(6093); |
| | | ZNF24(7572); MAP3K10(4294); NALP2(55655); STK4(6789); CSE1L(1434); |
| | | RAB22A(57403); BCL2L1(598); SYCP2(10388); DIDO1(11083); USP16 |
| | | (10600); SLC5A3(6526); BID(637); MN1(4330); PDGFB(5155); TOB2(10766); |
| | | TAF1(6872); RP6-213H19.1(51765); PHF6(84295); CD40LG(959); GPM6B |
| | | (2824); TAB3(257397); SMC1L1(8243); SPIN2(54466); RP11-130N24.1 |
| | | (340533); ARHGEF6(9459); CETN2(1069); |
| hsa-miR-504 | 103; 801; | PLEKHN1(84069); DFFB(1677); ZBTB40(9923); SESN2(83667); RBBP4 |
| | | (5928); MACF1(23499); NFYC(4802); RAP1A(5906); BCL9(607); S100A7L1 |
| | | (338324); UHMK1(127933); FASLG(356); MDM4(4194); C1orf69(200205); |
| | | TNFRSF18(8784); CCNL2(81669); TNFRSF14(8764); TNFRSF9(3604); |
| | | CTNNBIP1(56998); DFFA(1676); CASP9(842); KIAA0319L(79932); TESK2 |
| | | (10420); RHOC(389); PYGO2(90780); DEDD(9191); PRKCE(5581); HTRA2 |
| | | (27429); HK2(3099); UGCGL1(56886); CFLAR(8837); FZD7(8324); |
| | | COL4A3(1285); BOK(666); E2F6(1876); HS1BP3(64342); PPM1G(5496); |
| | | TIA1(7072); TFCP2L1(29842); PCAF(8850); TDGF1(6997); RNF7(9616); |
| | | RASSF1(11186); GSK3B(2932); HD(3064); EREG(2069); GAK(2580); SNCA |
| | | (6622); LEF1(51176); SPATA4(132851); KIAA1909(153478); TCF7(6932); |
| | | CDC23(8697); CDC25C(995); FGF1(2246); CSNK1A1(1452); CD74(972); |
| | | RIPK1(8737); SCUBE3(222663); MAPK13(5603); CDKN1A(1026); PARC |
| | | (23113); VEGF(7422); MDC1(9656); MDGA1(266727); KCNK5(8645); |
| | | MCM3(4172); DST(667); CCNC(892); PLAGL1(5325); LATS1(9113); SEPT7 |
| | | (989); FZD9(8326); FZD1(8321); TRRAP(8295); CUL1(8454); CDK6(1021); |
| | | UNC5D(137970); PINX1(54984); LZTS1(11178); TNFRSF10A(8797); |
| | | ADRA1A(148); MOS(4342); TP53INP1(94241); NUDT2(318); DAPK1(1612); |
| | | ABL1(25); TRAF2(7186); CDKN2A(1029); TAF1L(138474); IGFBPL1 |
| | | (347252); SEMA4D(10507); PTCH(5727); TRAF1(7185); TSC1(7248); C9orf37 |
| | | (85026); ZMYND11(10771); SGPL1(8879); LZTS2(84445); SUFU(51684); |
| | | PDCD11(22984); CSPG6(9126); PARD3(56288); ZWINT(11130); DLG5 |
| | | (9231); SFRP5(6425); SEC31L2(25956); MKI67(4288); STIM1(6786); EXT2 |
| | | (2132); INCENP(3619); LGALS12(85329); CCND1(595); TAGLN(6876); |
| | | IGF2(3481); MAP3K11(4296); BRMS1(25855); PPP1CA(5499); FGF3 |
| | | (2248); FZD4(8322); BCL9L(283149); PTPN6(5777); BCL2L14(79370); FLJ20489 |
| | | (55652); ESPL1(9700); CDK2(1017); MDM2(4193); APAF1(317); TMEM132B |
| | | (114795); FMNL3(91010); FAIM2(23017); CDK2AP1(8099); SCARB1 |
| | | (949); CHFR(55743); LATS2(26524); CSNK1A1L(122011); LIG4(3981); |
| | | TXNDC(81542); TRAF3(7187); ACIN1(22985); NEK9(91754); CHES1 |
| | | (1112); PPP1R13B(23368); KLF13(51621); ARNT2(9915); IL16(3603); NDN |
| | | (4692); BMF(90427); FLJ43339(388115); CSNK1G1(53944); PDCD7 |
| | | (10081); FLJ22795(80154); TRAF7(84231); KREMEN2(79412); PRRT2(112476); |
| | | PAPD5(64282); CARD15(64127); CYLD(1540); PKMYT1(9088); C16orf5 |
| | | (29965); ERN2(10595); P8(26471); TRADD(8717); SLC12A4(6560); |
| | | SMPD3(55512); TNFSF12(8742); ALOX15B(247); LIG3(3980); STARD3 |
| | | (10948); NME2(4831); PRKCA(5578); GPS1(2873); MNT(4335); P2RX1(5023); |
| | | TP53(7157); GAS7(8522); PHB(5245); MPO(4353); TK1(7083); MAPRE2 |
| | | (10982); CCDC5(115106); MAPK4(5596); COLEC12(81035); ESCO1(114799); |
| | | BCL2(596); MBP(4155); PARD6G(84552); FZR1(51343); ARHGEF18 |
| | | (23370); PIN1(5300); DNM2(1785); NDUFA13(51079); PSMD8(5714) |

Table 4-67

| | | |
|---|---|---|
| | | ; AXL(558); APOE(348); SPHK2(56848); BCL2L12(83596); DEDD2(162989); |
| | | PPP1R13L(10848); CARD8(22900); TRIB3(57761); CEP250(11190); |
| | | CTNNBL1(56259); STK4(6789); BCL2L1(598); BAK1(578); PTPRT(11122); |
| | | CABLES2(81928); DIDO1(11083); BTBD4(140685); APP(351); SNF1LK |
| | | (150094); ITGB2(3689); POFUT2(23275); NF2(4771); MCM5(4174); APOL6 |
| | | (80830); MAP3K7IP1(10454); EP300(2033); YPEL1(29799); LIF(3976); |
| | | SSTR3(6753); TOB2(10766); MAPK12(6300); PCTK1(5127); MAGEH1(28986); |
| | | TAF1(6872); SEPT6(23157); |
| hsa-miR-505 | 104; 802; | TNFRSF1B(7133); CDC42(998); RCC1(1104); KHDRBS1(10657); MACF1 |
| | | (23499); GADD45A(1647); RAP1A(5906); RGS2(5997); ADORA1(134); TGFB2 |
| | | (7042); E2F2(1870); FAF1(11124); SASS6(163786); NOTCH2(4853); |
| | | MAPRE3(22924); BIRC6(57448); MSH2(4436); PROC(5624); RIF1(55183); |
| | | ZAK(51776); CD28(940); COL4A3(1285); RTN4(57142); TIA1(7072); BUB1 |
| | | (699); IL1A(3552); IL1B(3553); MCM6(4175); FIGN(55137); TLK1(9874); |
| | | NCKAP1(10787); PAX3(5077); CUL3(8452); HDAC4(9759); FANCD2 |
| | | (2177); RBM5(10181); NEK11(79858); ATR(545); HD(3064); CD38(952); IL8 |
| | | (3576); FGF5(2250); APBB2(323); KDR(3791); SNCA(6622); MAD2L1 |
| | | (4085); CASP3(836); NIPBL(25836); GZMA(3001); ERBB2IP(55914); MAN2A1 |
| | | (4124); APC(324); CSNK1G3(1456); SLC12A2(6558); SLC26A2(1836); |
| | | GALNT10(55568); ATG12(9140); PPP2CA(5515); FGF1(2246); DUSP1 |
| | | (1843); CDKN1A(1026); PTP4A1(7803); TNFAIP3(7128); SUPT3H(8464); |
| | | MCM3(4172); DST(667); MAP3K5(4217); PDCD2(5134); AHR(196); HDAC9 |
| | | (9734); CASP2(835); TRIAD3(54476); CDK6(1021); TNFRSF10C(8794); |
| | | BNIP3L(665); UNC5D(137970); FKSG2(59347); UBE2V2(7336); EBAG9 |
| | | (9166); RAD21(5885); EXT1(2131); NTRK2(4915); PALM2-AKAP2(445815); |
| | | BAG1(573); GAS1(2619); SEMA4D(10507); TNFSF8(944); TRAF1(7185); |
| | | TSC1(7248); ZMYND11(10771); C10orf9(219771); PTEN(5728); ENTPD7 |
| | | (57089); BTRC(8945); NOLC1(9221); PDCD11(22984); DOCK1(1793); |
| | | PARD3(56288); FZD8(8325); DLG5(9231); HIPK3(10114); CCND1(595); |
| | | CUL5(8065); ATM(472); LOH11CR2A(4013); WT1(7490); HRASLS3(11145); |
| | | PAK1(5058); MDM2(4193); CCT2(10576); APAF1(317); RAN(5901); BCAT1 |
| | | (586); KRAS(3845); CBX5(23468); WIF1(11197); PAWR(5074); MPHO |
| | | SPH9(10198); SCARB1(949); FGF9(2254); BRCA2(675); CDC16(8881); FOXO1A |
| | | (2308); NEK3(4752); LIG4(3981); BCL2L2(599); CGRRF1(10668); |
| | | C14orf118(55668); ACIN1(22985); STRN3(29966); EGLN3(112399); CHES1 |
| | | (1112); BCL11B(64919); PPP1R13B(23368); JAG2(3714); BUB1B(701); |
| | | CSNK1G1(53944); PDCD7(10081); CYLD(1540); CTCF(10664); GAN |
| | | (8139); PKMYT1(9088); XYLT1(64131); ERN2(10595); SIAH1(6477); PCOLN3 |
| | | (5119); TRAF4(9618); NF1(4763); PPM1D(8493); PRKCA(5578); BECN1 |
| | | (8678); PTRH2(51651); SOCS3(9021); CSNK1D(1453); MALT1(10892); |
| | | ESCO1(114799); DNM2(1785); GRLF1(2909); TNFSF14(8740); MAPRE1 |
| | | (22919); MYBL2(4605); CSE1L(1434); C20orf11(54994); ELMO2(63916); |
| | | DIDO1(11083); SON(6651); ITGB2(3689); EP300(2033); TOB2(10766); CCNB3 |
| | | (85417); MLLT7(4303); AGTR2(186); BIRC4(331); BTK(695); CETN2 |
| | | (1069); |
| hsa-miR-506 | 105; 919; 920; | DFFB(1677); VAMP3(9341); H6PD(9563); INFRSF1B(7133); CDC42(998); |
| | 921; 922; | SESN2(83667); PLK3(1263); SH3GLB1(51100); CDC14A(8556); RAP1A |
| | 923; 924; | (5906); CD2(914); UHMK1(127933); LAMC1(3915); PPP1R12B(4660); TRAF5 |
| | | (7188); CIAS1(114548); CCNL2(81669); RCC2(55920); E2F2(1870); TESK2 |
| | | (10420); NRAS(4893); TRIM45(80263); NOTCH2(4853); MCL1(4170); |
| | | KIAA0040(9674); ASPM(259266); BIRC6(57448); TTC7A(57217); MAL |
| | | (4118); BCL2L11(10018); LOC339745(339745); RIF1(55183); TAIP-2(80034); |
| | | CD28(940); CTDSP1(58190); EIF2AK2(5610); RTN4(57142); SERTAD2 |
| | | (9792); TGFA(7039); SEPT10(151011); BUB1(699); ERCC3(2071); RAPH1 |
| | | (65059); PAX3(5077); CUL3(8452); HDAC4(9759); FANCD2(2177); |
| | | PDCD6IP(10015); LIMD1(8994); FXR1(8087); KLHL24(54800); TRAIP(10293); |
| | | RASSF1(11186); IL17RD(54756); MAGI1(9223); KLF15(28999); PIK3CB |
| | | (5291); CLDN1(9076); IL8(3576); SEPT11(55752); CCNG2(901); FLJ20032 |
| | | (54790); BTC(685); UNC5C(8633); PDCD6(10016); SEP2(6502); NIPBL |
| | | (25836); RAD17(5884); APC(324); RAD50(10111); SMAD5(4090); UNC5A |
| | | (90249); PRLR(5618); MCC(4163); SEMA6A(57556); IRF1(3659); DDX41 |
| | | (51428); E2F3(1871); ZNF192(7745); HLA-E(3133); LTA(4049); TNF |
| | | (7124); PARC(23113); FOXO3A(2309); SASH1(23328); QKI(9444); MDC1 |
| | | (9656); BAK1(578); CCND3(896); CUL7(9820); TNFRSF21(27242); DST(667); |
| | | SGK(6446); KATNA1(11104); PDCD2(5134); AHR(196); HDAC9(9734 |

Table 4-68

| | | |
|---|---|---|
| | | ); RINT1(60561); TRIAD3(54476); CDK6(1021); UNC5D(137970); SULF1 |
| | | (23213); E2F5(1875); RIPK2(8767); FZD6(8323); WISP1(8840); LZTS1(11178); |
| | | SNAI2(6591); TP53INP1(94241); TNFRSF11B(4982); ANKRD15(23189); |
| | | JAK2(3717); DAPK1(1612); C9orf47(286223); CDK9(1025); FPGS |
| | | (2356); ABL1(25); TAF1L(138474); B4GALT1(2683); KIAA1161(57462); PTCH |
| | | (5727); TNFSF15(9966); TNFSF8(944); PHF19(26147); TSC1(7248); |
| | | ANAPC2(29882); C10orf9(219771); SGPL1(8879); UNC5B(219699); KIF11 |
| | | (3832); PARD3(56288); ZWINT(11130); DLG5(9231); SFRP5(6425); SH3PXD2A |
| | | (9644); C10orf46(143384); TIAL1(7073); BNIP3(664); TUB(7275); |
| | | GAS2(2620); HIPK3(10114); MDK(4192); LRP5(4041); ATM(472); CBL(867); |
| | | LOH11CR2A(4013); RHOG(391); DKK3(27122); BAD(572); RELA(5970); |
| | | FZD4(8322); SESN3(143686); ZW10(9183); BCL9L(283149); CCND2 |
| | | (894); NTF3(4908); TNFRSF7(939); BCL2L14(79370); IAPP(3375); PDE1B |
| | | (5153); PA2G4(5036); MDM2(4193); SOCS2(8835); BCAT1(586); KRAS |
| | | (3845); LALBA(3906); MCRS1(10445); ANAPC7(51434); DIABLO(56616); |
| | | MPHOSPH9(10198); SCARB1(949); CHFR(55743); TNFRSF19(55504); |
| | | BRCA2(675); CUL4A(8451); LATS2(26524); NEK3(4752); PSEN1(5663); |
| | | VASH1(22846); YY1(7528); TRAF3(7187); SIVA(10572); DLG7(9787); SIX4 |
| | | (51804); LTBP2(4053); PGF(5228); NEK9(91754); BAG5(9529); JAG2(3714); |
| | | BUB1B(701); CSK(1445); ZSCAN2(54993); SLC12A6(9990); BMF(90427); |
| | | PRTG(283659); PDCD7(10081); CARD15(64127); CYLD(1540); RBL2 |
| | | (5934); ELMO3(79767); PSKH1(5681); AXIN1(8312); APPBP1(8883); SMPD3 |
| | | (55512); PCOLN3(5119); HIC1(3090); PAFAH1B1(5048); TNFSF12 |
| | | (8742); MAPK7(5598); TRAF4(9618); NF1(4763); MAP2K6(5608); CDK3 |
| | | (1018); SPHK1(8877); ANAPC11(51529); METT10D(79066); NALP1(22861); |
| | | FXR2(9513); AURKB(9212); HDAC5(10014); MPO(4353); ERN1(2081); |
| | | MALT1(10892); ROCK1(6093); UHRF1(29128); PIN1(5300); DNM2(1785); |
| | | CASP14(23581); ARHGEF1(9138); APOE(348); SPHK2(56848); BAX |
| | | (581); PPP2R1A(5518); NALP2(55655); AES(166); RAB3D(9545); DNASE2 |
| | | (1777); CEBPA(1050); ERF(2077); PPP1R13L(10848); TRIB3(57761); MCM8 |
| | | (84515); PLCB1(23236); CEP250(11190); STK4(6789); PARD6B(84612); |
| | | RBL1(5933); AURKA(6790); CABLES2(81928); DIDO1(11083); CHAF1B |
| | | (8208); BTG3(10950); SNF1LK(150094); BCL2L13(23786); LGALS1 |
| | | (3956); TNRC6B(23112); BID(637); MAPK1(5594); PPM1F(9647); MN1(4330); |
| | | SSTR3(6753); HDAC6(10013); TAF1(6872); RP6-213H19.1(51765); DKC1 |
| | | (1736); FIGF(2277); ARHGEF6(9459); |
| hsa-mir-507 | 106; 925; | DFFB(1677); KHDRBS1(10657); MACF1(23499); NFYC(4802); CD2(914); |
| | | CKS1B(1163); DAP3(7818); IL19(29949); CIAS1(114548); TNFRSF9(3604); |
| | | RCC2(55920); BCL10(8915); NOTCH2(4853); DUSP10(11221); TP53BP2 |
| | | (7159); RHOB(388); PPP1CB(5500); BIRC6(57448); PRKCE(5581); STEAP3 |
| | | (55240); RAB3GAP1(22930); RIF1(55183); ZAK(51776); TIA1(7072); |
| | | ANAPC1(64682); MGC4268(83607); SCN3A(6328); SCN1A(6323); BARD1 |
| | | (580); RBMS3(27303); MCM2(4171); KNG1(3827); TP73L(8626); OXTR |
| | | (5021); TADA3L(10474); PIK3CB(5291); CD38(952); LEF1(51176); PDCD6 |
| | | (10016); APC(324); RAD50(10111); TCF7(6932); PRLR(5618); PRKAA1 |
| | | (5562); MCC(4163); CDC23(8697); RNF130(55819); E2F3(1871); ZNF192 |
| | | (7745); TCF19(6941); CSNK2B(1460); CDC5L(988); IL17(3605); PTP4A1 |
| | | (7803); FOXO3A(2309); IHPK3(117283); DST(667); BACH2(60468); MAP3K5 |
| | | (4217); LATS1(9113); AHR(196); HBP1(26959); CUL1(8454); SOSTDC1 |
| | | (25928); INHBA(3624); LOC401357(401357); CDK6(1021); ZC3HC1(51530); |
| | | PTN(5764); UNC5D(137970); UBE2V2(7336); BIN3(55909); TNFRSF10D |
| | | (8793); TNFRSF10A(8797); TRIM35(23087); CLU(1191); TNFRSF11B |
| | | (4982); CDC37L1(55664); INVS(27130); TRAF2(7186); CDKN2B(1030); |
| | | BAG1(573); PTCH(5727); TSC1(7248); ANAPC2(29882); DKK1(22943); |
| | | SIRT1(23411); FAS(355); FRAT1(10023); DOCK1(1793); IL2RA(3559); FRAT2 |
| | | (23401); TIAL1(7073); MKI67(4288); BNIP3(664); TEAD1(7003); HIPK3 |
| | | (10114); EXT2(2132); INCENP(3619); LGALS12(85329); DPF2(5977); |
| | | SIPA1(6494); ATM(472); CHEK1(1111); CDKN1C(1028); EIF4G2(1982); |
| | | DKK3(27122); HRASLS3(11145); CDCA5(113130); FZD4(8322); CCND2 |
| | | (894); NTF3(4908); TNFRSF7(939); CNAP1(9918); MDM2(4193); SOCS2 |
| | | (8835); KNTC1(9735); TMEM132B(114795); FZD10(11211); BCAT1(586); |
| | | BTG1(694); HRK(8739); CHFR(55743); FGF9(2254); RB1(5925); RFP2(10206); |
| | | ING1(3621); CUL4A(8451); FOXO1A(2308); PNN(5411); GPR65(8477); |
| | | MAMDC1(161357); BAG5(9529); FGF7(2252); IGF1R(3480); AVEN(57099); |
| | | TRAF7(84231); CCNF(899); DNASE1(1773); CARD15(64127); AXI |

Table 4-69

| | | |
|---|---|---|
| | | N1(8312); GSPT1(2935); PAFAH1B1(5048); NF1(4763); LIG3(3980); MAPT |
| | | (4137); RPS6KB1(6198); CDK3(1018); ANAPC11(51529); MNT(4335); P2RX1 |
| | | (5023); NALP1(22861); SPAG5(10615); CBX4(8535); TBC1D16(125058); |
| | | DCC(1630); PARD6G(84552); CHAF1A(10036); ARHGEF1(9138); DPF1 |
| | | (8193); PLCB1(23236); RAB22A(57403); PCNA(5111); RBL1(5933); CTCFL |
| | | (140690); SYCP2(10388); SON(6651); SLC5A3(6526); APP(351); CRYZL1 |
| | | (9946); EP300(2033); PPM1F(9647); AGTR2(186); BIRC4(331); STAG2 |
| | | (10735); RP6-213H19.1(51765); DDX26B(203522); FIGF(2277); ARHGEF6 |
| | | (9459); |
| hsa-miR-509 | 107; | UTP11L(51118); PRKACB(5567); FAF1(11124); DDAH1(23576); NOTCH2 |
| | | (4853); DEDD(9191); ASPM(259266); LPGAT1(9926); PCNXL2(80003); |
| | | CREB1(1385); TGFA(7039); IL1A(3552); CACNB4(785); PDCD6IP(10015); |
| | | LIMD1(8994); APPL(26060); ADIPOQ(9370); KIAA1143(57456); LRRC15 |
| | | (131578); RNF4(6047); HCAP-G(64151); TMPRSS11E(28983); FGF5(2250); |
| | | PLK4(10733); CARD6(84674); APC(324); PRLR(5618); FLJ44796(401209); |
| | | IRF1(3659); HTR4(3360); RNF130(55819); IL17(3605); SLC22A3 |
| | | (6581); SERPINB9(5272); ATXN1(6310); DHX16(8449); DST(667); LATS1 |
| | | (9113); EGFR(1956); ZNF588(51427); BCAP29(55973); MMD2(221938); PMS2 |
| | | (5395); INHBA(3624); H-plk(51351); PTK2B(2185); SULF1(23213); |
| | | RIPK2(8767); ADRA1A(148); CLU(1191); TP53INP1(94241); NCALD(83988); |
| | | MTSS1(9788); UNC13B(10497); C10orf9(219771); PTEN(5728); LZTS2 |
| | | (84445); SUFU(51684); TCF7L2(6934); SH3PXD2A(9644); C10orf46 |
| | | (143384); GAS2(2620); HIPK3(10114); F2(2147); YAP1(10413); ATM(472); |
| | | SLC37A2(219855); PPP2R1B(5519); BCL9L(283149); SSPN(8082); TEGT |
| | | (7009); ESPL1(9700); CDK2(1017); APAF1(317); KNTC1(9735); LRP6(4040); |
| | | ARF3(377); C12orf22(81566); DUSP6(1848); PCTK2(5128); MPHOSPH9 |
| | | (10198); MGC7036(196383); BRCA2(675); RFP2(10206); ING1(3621); |
| | | PSEN1(5663); GPR65(8477); ACIN1(22985); GZMB(3002); DLG7(9787); |
| | | SYNJ2BP(55333); LTBP2(4053); DIO2(1734); CHES1(1112); BAG5(9529); |
| | | SCG3(29106); LOC440295(440295); IGF1R(3480); DAPK2(23604); CSNK1G1 |
| | | (53944); FLJ21736(79984); GSPT1(2935); SLC12A4(6560); SMPD3 |
| | | (55512); ST3GAL2(6483); MYOCD(93649); NLK(51701); LIG3(3980); GAS7 |
| | | (8522); RAB5C(5878); HDAC5(10014); CSNK1D(1453); GIPC3(126326); |
| | | FOSB (2354); ELAVL1(1994); OSCAR(126014); MCM8(84515); CEP250 |
| | | (11190); RBL1(5933); SON(6651); BRWD1(54014); BCL2L13(23786); PDXP |
| | | (57026); DMC1(11144); AGTR2(186); RP11-308D16.4(441522); RPL10(6134); |
| | | SMC1L1(8243); AMOT(154796); RBMY1A1(5940); |
| hsa-miR-510 | 108; | TP73(7161); TNFRSF1B(7133); SESN2(83667); RCC1(1104); RBBP4(5928); |
| | | MACF1(23499); CDC20(991); GADD45A(1647); CD2(914); BCL9(607); |
| | | CKS1B(1163); PEA15(8682); UHMK1(127933); MDM4(4194); TRAF5(7188); |
| | | C1orf95(375057); CIAS1(114548); TNFRSF9(3604); CTNNBIP1(56998); |
| | | CASP9(842); E2F2(1870); KIAA0319L(79932); MUTYH(4595); TESK2 |
| | | (10420); MCL1(4170); BIRC6(57448); HTRA2(27429); HK2(3099); KCNIP3 |
| | | (30818); RIF1(55183); CFLAR(8837); CASP10(843); EIF2AK2(5610); TMEM127 |
| | | (55654); TLK1(9874); STK17B(9262); PAX3(5077); FLJ43879(401039); |
| | | FANCD2(2177); VHL(7428); ZNF651(92999); TREX1(11277); MCM2 |
| | | (4171); NEK11(79858); KNG1(3827); TP73L(8626); TADA3L(10474); |
| | | CIDEC(63924); AXUD1(64651); STAG1(10274); CD38(952); CCNG2(901); |
| | | FGF5(2250); FGF2(2247); SFRP2(6423); SKP2(6502); UTP15(84135); APC |
| | | (324); UNC5A(90249); PRKAAI(5562); BIRC1(4671); ATG12(9140); SEMA6A |
| | | (57556); KIAA1961(96459); CSNK1A1(1452); FBXW11(23291); DUSP1 |
| | | (1843); RIPK1(8737); MAPK13(5603); PARC(23113); SLC22A7(10864); |
| | | ANKRD6(22881); PHACTR2(9749); SASH1(23328); NEDD9(4739); MDC1 |
| | | (9656); HLA-DOA(3111); CUL7(9820); PLAGL1(5325); SEPT7(989); |
| | | EGFR(1956); STAG3(10734); CUL1(8454); CYCS(54205); CDK6(1021); SLC7A2 |
| | | (6542); POLR3D(661); TNFRSF10C(8794); BNIP3L(665); PTK2B |
| | | (2185); UNC5D(137970); TERF1(7013); LZTS1(11178); BIN3(55909); TNFRSF10D |
| | | (8793); DUSP4(1846); SFRP1(6422); PAG1(55824); EXT1(2131); |
| | | MTSS1(9788); UNC13B(10497); NTRK2(4915); DAPK1(1612); C9orf47(286223); |
| | | INVS(27130); GPR107(57720); TRAF2(7186); BAG1(573); SEMA4D |
| | | (10507); TRAF1(7185); ANAPC2(29882); SIRT1(23411); SGPL1(8879); |
| | | SCD(6319); NOLC1(9221); SUFU(51684); DOCK1(1793); MTG1(92170); IL2RA |
| | | (3559); CUL2(8453); ZWINT(11130); DLG5(9231); SMNDC1(10285); |
| | | C10orf46(143384); EXT2(2132); INCENP(3619); LRP5(4041); CUL5(8065); |
| | | ATM(472); DUSP8(1850); IGF2(3481); CDCA5(113130); FGF3(2248); |

Table 4-70

| | | |
|---|---|---|
| | | SESN3(143686); CASP4(837); CASP5(838); LTBR(4055); BCL2L14(79370); |
| | | APOLD1(81575); AQP2(359); ESPL1(9700); PDE1B(5153); MDM2(4193); |
| | | SOCS2(8835); CRADD(8738); KNTC1(9735); TNFRSF1A(7132); NOL1 |
| | | (4839); BCAT1(586); KRAS(3845); HDAC7A(51564); LALBA(3906); FAIM2 |
| | | (23017); C12orf22(81566); RNF41(10193); ANAPC7(51434); SCARB1 |
| | | (949); CHFR(55743); ZNF605(90462); HSMPP8(54737); RFP2(10206); SUGT1 |
| | | (10910); CUL4A(8451); FOXO1A(2308); NEK3(4752); LIG4(3981); MNAT1 |
| | | (4331); PSEN1(5663); TRAF3(7187); ACIN1(22985); CDKL1(8814); |
| | | LTBP2(4053); NEK9(91754); CHES1(1112); C14orf143(90141); MOAP1(64112); |
| | | BAG5(9529); AKT1(207); KIAA1794(55215); IGF1R(3480); SLC12A6 |
| | | (9990); BMF(90427); BNIP2(663); CSNK1G1(53944); TSC2(7249); DNASE1 |
| | | (1773); PLK1(5347); PKMYT1(9098); GRIN2A(2903); ERN2(10595); |
| | | CD2BP2(10421); SEPT1(1731); TXNL4B(54957); TADA2L(6871); MAPT |
| | | (4137); PPM1D(8493); PRKCA(5578); BIRC5(332); CARD14(79092); ANAPC11 |
| | | (51529); NALP1(22861); GAS2L2(246176); ERN1(2081); ACOX1(51); |
| | | SOCS3(9021); RAB12(201475); CABLES1(91768); MAPK4(5596); DCC |
| | | (1630); PHLPP(23239); BCL2(596); PARD6G(84552); CDC34(997); UHRF1 |
| | | (29128); TNFSF9(8744); OR7D2(162998); MAP3K10(4294); AXL(558); APOE |
| | | (348); SPHK2(56848); BAX(581); TNFSF14(8740); DPF1(8193); DEDD2 |
| | | (162989); CARD8(22900); KLK10(5655); ZFPL(162967); MCM8(84515); |
| | | MYBL2(4605); PARD6B(84612); GGTL3(2686); PTGIS(5740); AURKA |
| | | (6790); SON(6651); SNF1LK(150094); BCL2L13(23786); SEPT5(5413); NF2 |
| | | (4771); MCM5(4174); EP300(2033); SEPT3(55964); CHEK2(11200); CARD10 |
| | | (29775); POLR3H(171568); FLJ41993(400935); RBM3(5935); MLLT7 |
| | | (4303); BIRC4(331); RP6-213H19.1(51765); PPP2R3B(28227); |
| hsa-miR-511 | 109; | SFN(2810); RCC1(1104); KHDRBS1(10657); RBBP4(5928); MACF1(23499); |
| | | CDC14A(8556); SYCP1(6847); BNIPL(149428); PEA15(8682); UHMK1 |
| | | (127933); IL24(11009); TRAF5(7188); CDC2L2(985); TNFRSF14(8764); |
| | | FRAP1(2475); CLSPN(63967); ITGB3BP(23421); TMED5(50999); NOTCH2 |
| | | (4853); DEDD(9191); TNFSF18(8995); C1orf24(116496); ASPM(259266); |
| | | TP53BP2(7159); BIRC6(57448); PRKCE(5581); MSH2(4436); CCT7(10574); |
| | | HK2(3099); TCF7L1(83439); BRRN1(23397); LOC339745(339745); |
| | | RIF1(55183); ZAK(51776); CASP10(843); ALS2CR19(117583); COL4A3(1285); |
| | | PSMD1(5707); SEPT2(4735); YWHAQ(10971); EIF2AK2(5610); RTN4 |
| | | (57142); TIA1(7072); SEPT10(151011); PAX8(7849); MCM6(4175); STAT1 |
| | | (6772); PAX3(5077); CUL3(8452); DTYMK(1841); FANCD2(2177); VHL |
| | | (7428); CCR5(1234); RBM5(10181); RBM15B(29890); GNL3(26354); ACPP |
| | | (55); FAIM(55179); SMC4L1(10051); FXR1(8087); PSMD2(5708); KNG1 |
| | | (3827); TP73L(8626); TUSC4(10641); FHIT(2272); GSK3B(2932); STAG1 |
| | | (10274); TFDP2(7029); ATR(545); SIAH2(6478); HCAP-G(64151); ANAPC4 |
| | | (29945); IL8(3576); SEPT11(55752); FGF2(2247); PLK4(10733); GAK |
| | | (2580); APBB2(323); PPBP(5473); PRKG2(5593); SNCA(6622); LEF1(51176); |
| | | SFRP2(6423); VEGFC(7424); PDCD6(10016); NIPBL(25836); CARD6 |
| | | (84674); ERBB2IP(55914); RAD17(5884); APC(324); CSNK1G3(1456); RAD50 |
| | | (10111); SGCD(6444); DAP(1611); PRLR(5618); BIRC1(4671); MCC(4163); |
| | | SEMA6A(57556); CDC25C(995); E2F3(1871); CSNK2B(1460); VEGF |
| | | (7422); CDC5L(988); IL17(3605); TNFAIP3(7128); HECA(51696); SASH1 |
| | | (23328); QKI(9444); NEDD9(4739); MDC1(9656); IHPK3(117283); SUPT3H |
| | | (8464); DST(667); FRK(2444); BCLAF1(9774); MAP3K5(4217); LATS1 |
| | | (9113); PDCD2(5134); AHR(196); HDAC9(9734); EGFR(1956); BCAP29(55973); |
| | | PMS2(5395); INHBA(3624); IGFBP3(3486); CDK6(1021); CNOT4 |
| | | (4850); HIPK2(28996); FZD3(7976); BAG4(9530); TACC1(6867); UBE2V2 |
| | | (7336); SULF1(23213); TERF1(7013); FZD6(8323); MYC(4609); PINX1(54984); |
| | | BIN3(55909); TRIM35(23087); DUSP4(1846); DKK4(27121); PAG1 |
| | | (55824); CCNE2(9134); TP53INP1(94241); STK3(6788); RAD21(5885); CDC37L1 |
| | | (55664); JAK2(3717); MTAP(4507); RECK(8434); NTRK2(4915); DAPK1 |
| | | (1612); INVS(27130); ABL1(25); TAF1L(138474); BAG1(573); TNFSF8 |
| | | (944); TSC1(7248); ABO(28); ZMYND11(10771); C10orf9(219771); DKK1 |
| | | (22943); SGPL1(8879); PTEN(5728); KIF11(3832); BTRC(8945); NOLC1 |
| | | (9221); SUFU(51684); CSPG6(9126); ACADSB(36); DOCK1(1793); IL2RA |
| | | (3559); PARD3(56288); CUL2(8453); ZWINT(11130); DLG5(9231); DCLRE1A |
| | | (9937); TIAL1(7073); MKI67(4288); STIM1(6786); EXT2(2132); CCND1 |
| | | (595); BIRC3(330); CUL5(8065); ATM(472); LOH11CR2A(4013); CHEK1 |
| | | (1111); IGF2(3481); PTH(5741); WT1(7490); FADS1(3992); HRASLS3 |
| | | (11145); MAP3K11(4296); BRMS1(25855); MTL5(9633); FGF4(2249); FZ |

Table 4-71

| | | |
|---|---|---|
| | | D4(8322); CTSC(1075); SESN3(143686); PTPRO(5800); IAPP(3375); MDM2 |
| | | (4193); SOCS2(8835); APAF1(317); GAS2L3(283431); KNTC1(9735); FZD10 |
| | | (11211); NOL1(4839); ING4(51147); KRAS(3845); LALBA(3906); PAWR |
| | | (5074); DUSP6(1848); BTG1(694); HRK(8739); ANAPC5(51433); MPHOSPH9 |
| | | (10198); HSMPP8(54737); FGF9(2254); BRCA2(675); RFP2(10206); |
| | | CUL4A(8451); TFDP1(7027); CDC16(8881); LATS2(26524); CSNK1A1L |
| | | (122011); FOXO1A(2308); LIG4(3981); PNN(5411); TXNDC(81542); CGRRF1 |
| | | (10668); GPR65(8477); STRN3(29966); CDKL1(8814); LTBP2(4053); |
| | | NEK9(91754); TGFB3(7043); CHES1(1112); BUB1B(701); MAPK6(5597); |
| | | KIF23(9493); AVEN(57099); SLC12A6(9990); BMF(90427); BNIP2(663); |
| | | CSNK1G1(53944); TSC2(7249); RBL2(5934); GSPT1(2935); XYLT1(64131); |
| | | FTS(64400); CSNK2A2(1459); MPHOSPH6(10200); PAFAH1B1(5048); |
| | | ALOX12(239); ALOX15B(247); NLK(51701); NF1(4763); MAPT(4137); |
| | | CROP(51747); TLK2(11011); PRKCA(5578); KPNA2(3838); NALP1(22861); |
| | | GAS7(8522); PLXDC1(57125); BRCA1(672); MAPRE2(10982); MAPK4 |
| | | (5596); MALT1(10892); PHLPP(23239); SERPINB2(5055); ROCK1(6093); |
| | | ESCO1(114799); TXNL1(9352); CASP14(23581); CCNE1(898); PDCD5 |
| | | (9141); ZNF226(7769); PPP2R1A(5518); NALP2(55655); AES(166); ELAVL3 |
| | | (1995); MGC71805(403113); ZNF443(10224); DPF1(8193); CARD8(22900); |
| | | PLCB1(23236); CTNNBL1(56259); STK4(6789); CD40(958); RAB22A |
| | | (57403); BIRC7(79444); RBL1(5933); UBE2V1(7335); CTCFL(140690); |
| | | SYCP2(10388); DIDO1(11083); USP16(10600); SON(6651); SNF1LK(150094); |
| | | BCL2L13(23786); LGALS1(3956); EP300(2033); SEPT3(55964); GTSE1 |
| | | (51512); BID(637); MAPK1(5594); PPM1F(9647); SMC1L2(27127); |
| | | PCTK1(5127); TAF1(6872); BIRC4(331); STAG2(10735); CD40LG(959); DKC1 |
| | | (1736); SMC1L1(8243); BTK(695); CUL4B(8450); PDCD8(9131); ARHGEF6 |
| | | (9459); HCFC1(3054); MTCP1(4515); |
| hsa-miR-512-3p | 110; | TNFRSF1B(7133); PAX7(5081); NBL1(4681); NUDC(10726); RBBP4(5928); |
| | | SH3GLB1(51100); CDC14A(8556); BCL9(607); BNIPL(149428); DAP3 |
| | | (7818); UHMK1(127933); GOS2(50486); TRAF5(7188); DISC1(27185); CIAS1 |
| | | (114548); TNFRSF14(8764); TNFRSF9(3604); CTNNBIP1(56998); DFFA |
| | | (1676); FRAP1(2475); CASP9(842); E2F2(1870); GFI1(2672); NOTCH2 |
| | | (4853); PYGO2(90780); ASPM(259266); IL10(3586); TP53BP2(7159); MAPRE3 |
| | | (22924); BIRC6(57448); MSH2(4436); HTRA2(27429); HK2(3099); |
| | | TCF7L1(83439); BRRN1(23397); TAIP-2(80034); ZAK(51776); CFLAR(8837); |
| | | CASP10(843); CASP8(841); CD28(940); ALS2CR19(117583); INHA |
| | | (3623); COL4A3(1285); TGFA(7039); FLJ14397(84865); ANAPC1(64682); |
| | | MCM6(4175); FRZB(2487); NCKAP1(10787); STAT1(6772); FZD5(7855); |
| | | HDAC4(9759); FANCD2(2177); RBM5(10181); GNL3(26354); SENP2(59343); |
| | | TP73L(8626); RAF1(5894); THRB(7068); NME6(10201); STAG1(10274); |
| | | TFDP2(7029); MGC40579(256356); ATR(545); HD(3064); CD38(952); |
| | | HCAP-G(64151); SEPT11(55752); NFKB1(4790); FGF2(2247); TLR2(7097); |
| | | APBB2(323); LEF1(51176); CASP6(839); LOC152485(152485); NEK1(4750); |
| | | KIAA1909(153478); NIPBL(25836); ERBB2IP(55914); CCNB1(891); |
| | | F2R(2149); APC(324); RAD50(10111); TCF7(6932); UNC5A(90249); BIRC1 |
| | | (4671); MCC(4163); ATG12(9140); IRF1(3659); FGF1(2246); DUSP1(1843); |
| | | E2F3(1871); PARC(23113); SASH1(23328); IER3(8870); NUDT3(11165); |
| | | CCND3(896); CUL7(9820); TNFRSF21(27242); DST(667); SESN1(27244); |
| | | PLAGL1(5325); KATNA1(11104); PDCD2(5134); HDAC9(9734); EGFR |
| | | (1956); FZD1(8321); CASP2(835); CUL1(8454); MAD1L1(8379); TRIAD3 |
| | | (54476); PMS2(5395); CYCS(54205); CARD4(10392); TMED4(222068); |
| | | HIP1(3092); CREB3L2(64764); CLN8(2055); TNFRSF10C(8794); ESCO2 |
| | | (157570); UNC5D(137970); UBE2V2(7336); PINX1(54984); LZTS1(11178); |
| | | TNFRSF10B(8795); TNFRSF10D(8793); TNFRSF10A(8797); TRIM35 |
| | | (23087); FGFR1(2260); DKK4(27121); TP53INP1(94241); STK3(6788); |
| | | ANKRD15(23189); C9orf47(286223); INVS(27130); TAF1L(138474); BAG1 |
| | | (573); FANCG(2189); PTCH(5727); CTNNAL1(8727); TNFSF15(9966); TRAF1 |
| | | (7185); CIZ1(25792); TSC1(7248); C10orf9(219771); UNC5B(219699); |
| | | KIF11(3832); PDCD11(22984); BAG3(9531); DOCK1(1793); IL2RA(3559); |
| | | CUL2(8453); FZD8(8325); ZWINT(11130); DLG5(9231); SMNDC1(10285); |
| | | C10orf46(143384); BNIP3(664); GAS2(2620); HIPK3(10114); EXT2 |
| | | (2132); LGALS12(85329); SSSCA1(10534); FADD(8772); BIRC2(329); ATM |
| | | (472); APBB1(322); EIF4G2(1982); CDCA5(113130); MAP3K11(4296); |
| | | RELA(5970); PAK1(5058); BCL9L(283149); hCAP-D3(23310); CNAP1(9918); |
| | | LAPP(3375); MDM2(4193); KNTC1(9735); TNFRSF1A(7132); NOL1( |

Table 4-72

| | | |
|---|---|---|
| | | 4839); MCRS1(10445); FAIM2(23017); C12orf22(81566); CDK4(1019); CPM |
| | | (1368); PAWR(5074); DIP13B(55198); ANAPC7(51434); ANAPC5(51433); |
| | | MPHOSPH9(10198); CHFR(55743); HSMPP8(54737); TNFRSF19(55504); |
| | | BRCA2(675); RFP2(10206); CUL4A(8451); TFDP1(7027); FOXO1A |
| | | (2308); NEK3(4752); LIG4(3981); TXNDC(81542); VASH1(22846); C14orf153 |
| | | (84334); CHES1(1112); MOAP1(64112); PPP1R13B(23368); AKT1(207); |
| | | FGF7(2252); CSNK1G1(53944); TRAF7(84231); ZNF213(7760); DNASE1 |
| | | (1773); DNAJA3(9093); SPN(6693); RBL2(5934); MARVELD3(91862); CDT1 |
| | | (81620); AFG3L1(172); GSPT1(2935); ERN2(10595); FTS(64400); APPBP1 |
| | | (8883); TRADD(8717); SMPD3(55512); TXNL4B(54957); PCOLN3 |
| | | (5119); HIC1(3090); GSG2(83903); TNFSF12(8742); ALOX15B(247); NLK |
| | | (51701); NF1(4763); TADA2L(6871); FZD2(2535); MAPT(4137); TLK2(11011); |
| | | BIRC5(332); ANAPC11(51529); NALP1(22861); GAS7(8522); BRCA1 |
| | | (672); HDAC5(10014); TBC1D16(125058); CSNK1D(1453); CABLES1(91768); |
| | | MAPK4(5596); DCC(1630); TXNL1(9352); TXNL4A(10907); TNFAIP8L1 |
| | | (126282); DNM2(1785); F2RL3(9002); PIK3R2(5296); UBA52(7311); |
| | | CCNE1(898); PSMD8(5714); ATF5(22809); TNFSF7(970); TNFSF14(8740); |
| | | ZNF443(10224); DNASE2(1777); SIRT2(22933); PPP1R13L(10848); CARD8 |
| | | (22900); CEP250(11190); MYBL2(4605); C20orf121(79183); STK4 |
| | | (6789); BIRC7(79444); CSNK2A1(1457); RBL1(5933); ELMO2(63916); DIDO1 |
| | | (11083); SON(6651); MX1(4599); SNF1LK(150094); CDC45L(8318); |
| | | SEPT5(5413); KREMEN1(83999); GAS2L1(10634); SEPT3(55964); PPARA |
| | | (5465); BID(637); MAPK1(5594); PPM1F(9647); CARD10(29775); DMC1 |
| | | (11144); TOB2(10766); SMC1L2(27127); CCNB3(85417); MAGEH1(28986); |
| | | TAF1(6872); BIRC4(331); RP6-213H19.1(51765); CD40LG(959); CXorf38 |
| | | (159013); SEPT6(23157); CUL4B(8450); ARHGEF6(9459); BCAP31 |
| | | (10134); HCFC1(3054); |
| hsa-miR-512-5p | 111; | CDC42(998); SFN(2810); RCC1(1104); HDAC1(3065); RBBP4(5928); MACF1 |
| | | (23499); GADD45A(1647); BNIPL(149428); CKS1B(1163); PEA15(8682); |
| | | UHMK1(127933); TRAF5(7188); CIAS1(114548); TNFRSF14(8764); |
| | | CTNNBIP1(56998); FRAP1(2475); RCC2(55920); YARS(8565); CLSPN(63967); |
| | | MUTYH(4595); TESK2(10420); CD5L(922); ASPM(259266); TMEM63A |
| | | (9725); CCT7(10574); HTRA2(27429); TCF7L1(83439); KCNIP3(30818); |
| | | PDCL3(79031); RIF1(55183); CASP10(843); PTMA(5757); E2F6(1876); |
| | | FLJ34931(388939); RTN4(57142); CCT4(10575); TGFA(7039); EDAR |
| | | (10913); BUB1(699); ANAPC1(64682); STAT1(6772); HDAC4(9759); PDCD6IP |
| | | (10015); DLEC1(9940); CTNNB1(1499); TDGF1(6997); EAF2(55840); |
| | | MCM2(4171); NEK11(79858); FXR1(8087); PSMD2(5708); AXUD1(64651); |
| | | SLCO2A1(6578); HD(3064); HCAP-G(64151); EREG(2069); CCNG2 |
| | | (901); NFKB1(4790); PLK4(10733); GAK(2580); PRKG2(5593); LEF1(51176); |
| | | F2R(2149); RAD50(10111); UNC5A(90249); TERT(7015); PRLR(5618); |
| | | PRKAA1(5562); UNG2(10309); BIRC1(4671); MCC(4163); ATG12(9140); |
| | | IRF1(3659); FGF1(2246); FMXW11(23291); SH3PXD2B(285590); MAPK13 |
| | | (5603); CUL7(9820); MCM3(4172); DST(667); ATG5(9474); MAP3K5(427); |
| | | AGPAT4(56895); STK17A(9263); EGFR(1956); FZD9(8326); RINT1(60561); |
| | | HBP1(26959); MAD1L1(8379); TRIAD3(54476); PMS2(5395); CYCS |
| | | (54205); CARD4(10392); ELMO1(9844); IGFBP3(3486); CDK6(1021); ZC3HC1 |
| | | (51530); PTN(5764); FLJ40852(285962); DLGAP2(9228); PTK2B |
| | | (2185); UNC5D(137970); TACC1(6867); SULF1(23213); MYC(4609); GML |
| | | (2765); LZTS1(11178); TNFRSF10B(8795); STK3(6788); EXT1(2131); ANKRD15 |
| | | (23189); RECK(8434); CDK9(1025); ABL1(25); CDKN2B(1030); TAF1L |
| | | (138474); SEMA4D(10507); TRAF1(7185); CARD9(64170); WDR37 |
| | | (22884); PDCD11(22984); TCF7L2(6934); DLG5(9231); FBXW4(6468); EXT2 |
| | | (2132); MDK(4192); LRP5(4041); FADD(8772); CUL5(8065); LOH11CR2A |
| | | (4013); CDKN1C(1028); APBB1(322); EIF4G2(1982); HRASLS3(11145); |
| | | RELA(5970); SESN3(143686); IL18(3606); BCL9L(283149); TNFRSF7 |
| | | (939); CNAP1(9918); BCL2L14(79370); ESPL1(9700); KNTC1(9735); HDAC7A |
| | | (51564); MCRS1(10445); FAIM2(23017); BAZ2A(11176); DDIT3(1649); |
| | | LIN7A(8825); DUSP6(1848); DIABLO(56616); MPHOSPH9(10198); CUL4A |
| | | (8451); TFDP1(7027); LIG4(3981); BCL2L2(599); TRAF3(7187); ACIN1 |
| | | (22985); GZMB(3002); LTBP2(4053); BBS4(585); IGF1R(3480); BNIP2 |
| | | (663); DAPK2(23604); CCNF(899); CARD15(64127); PDPR(55066); AXIN1 |
| | | (8312); GSPT1(2935); ERN2(10595); MAPK3(5595); FTS(64400); SLC12A4 |
| | | (6560); PAFAH1B1(5048); TNFSF12(8742); MAPK7(5598); TRAF4 |
| | | (9618); LIG3(3980); MAPT(4137); PRKCA(5578); KPNA2(3838); CDK3(10 |

Table 4-73

| | | |
|---|---|---|
| | | 18); GPS1(2873); MNT(4335); METT10D(79066); GAS7(8522); SPAG5(10615); |
| | | GAS2L2(246176); BRCA1(672); HDAC5(10014); MALT1(10892); PHLPP |
| | | (23239); ROCK1(6093); FZR1(51343); PIK3R2(5296); PSMD8(5714); |
| | | APOE(348); CDKN2D(1032); C19orf12(83636); PRX(57716); DEDD2(162989); |
| | | MCM8(84515); MAPRE1(22919); CEP250(11190); CTNNBL1(56259); |
| | | BIRC7(79444); CSNK2A1(1457); BCL2L1(598); C20orf128(128876); CABLES2 |
| | | (81928); DIDO1(11083); BCL2L13(23786); ADORA2A(135); NF2 |
| | | (4771); MTP18(51537); PGEA1(25776); SEPT3(55964); GTSE1(51512); MAPK8IP2 |
| | | (23542); MAPK1(5594); MN1(4330); PDGFB(5155); TOB2(10766); |
| | | MAPK12(6300); HDAC6(10013); TAF1(6872); PPP2R3B(28227); SEPT6 |
| | | (23157); HCFC1(3054); |
| hsa-miR-513 | 112; | TNFRSF1B(7133); SFN(2810); HDAC1(3065); MACF1(23499); NFYC(4802); |
| | | CDKN2C(1031); GADD45A(1647); CDC7(8317); PROK1(84432); CD2 |
| | | (914); BCL9(607); BNIPL(149428); UHMK1(127933); C1orf21(81563); RGS2 |
| | | (5997); TGFB2(7042); TNFRSF9(3604); CTNNBIP1(56998); DFFA(1676); |
| | | ZBTB17(7709); SDC3(9672); AK2(204); CLSPN(63967); MUTYH(4595); |
| | | TESK2(10420); FAF1(11124); PPAP2B(8613); DDAH1(23576); NOTCH2 |
| | | (4853); RGS16(6004); GLRX2(51022); TNNI1(7135); IL10(3586); BIRC6 |
| | | (57448); HK2(3099); TCF7L1(83439); MAL(4118); BCL2L11(10018); ZAK |
| | | (51776); PMS1(5378); CASP8(841); FZD7(8324); CD28(940); HRB(3267); |
| | | BOK(666); PPM1G(5496); CARD12(58484); RTN4(57142); TGFA(7039); |
| | | EDAR(10913); SEPT10(151011); BUB1(699); ANAPC1(64682); BIN1(274); |
| | | MCM6(4175); IFIH1(64135); FRZB(2487); STAT1(6772); FANCD2(2177); |
| | | PCAF(8850); LIMD1(8994); PCNP(57092); NEK11(79858); ACPP(55); PSMD2 |
| | | (5708); CIDEC(63924); ALS2CL(259173); UCN2(90226); KIAA2018 |
| | | (205717); GSK3B(2932); LRRC15(131578); MGC21675(92070); WHSC1 |
| | | (7468); HD(3064); CD38(952); HCAP-G(64151); TLR2(7097); FAM53A(152877); |
| | | APBB2(323); CCNI(10983); UNC5C(8633); SFRP2(6423); VEGFC |
| | | (7424); NIPBL(25836); GZMA(3001); APC(324); UNC5A(90249); ZNF346 |
| | | (23567); PRLR(5618); PRKAA1(5562); MCC(4163); SEMA6A(57556); PPP2CA |
| | | (5515); CD74(972); FBXW11(23291); DDX41(51428); E2F3(1871); TNF |
| | | (7124); C2(717); KIAA0240(23506); CDC5L(988); TTK(7272); FOXO3A |
| | | (2309); TNFAIP3(7128); ZDHHC14(79683); RP11-145H9.1(340156); DHX16 |
| | | (8449); MDGA1(266727); CUL7(9820); TNFRSF21(27242); DST(667); LATS1 |
| | | (9113); PDCD2(5134); ADCY1(107); FZD9(8326); HBP1(26959); CUL1 |
| | | (8454); TRIAD3(54476); PMS2(5395); SOSTDC1(25928); INHBA(3624); |
| | | CDK6(1021); ZC3HC1(51530); CREB3L2(64764); HIPK2(28996); DLGAP2 |
| | | (9228); KIAA1967(57805); BNIP3L(665); UNC5D(137970); BAG4(9530); |
| | | TACC1(6867); SULF1(23213); FZD6(8323); WISP1(8840); ZFP41(286128); |
| | | BIN3(55909); TNFRSF10B(8795); TNFRSF10D(8793); SFRP1(6422); |
| | | TP53INP1(94241); RAD21(5885); EXT1(2131); MTSS1(9788); JAK2(3717); |
| | | NTRK2(4915); INVS(27130); ABL1(25); MOBKL2B(79817); TAF1L(138474); |
| | | FANCG(2189); SEMA4D(10507); PHF19(26147); CIZ1(25792); ANAPC2 |
| | | (29882); C9orf111(375775); ZMYND11(10771); C10orf9(219771); DKK1 |
| | | (22943); SIRT1(23411); PTEN(5728); BTRC(8945); NOLC1(9221); SUFU |
| | | (51684); DOCK1(1793); PARD3(56288); DLG5(9231); FBXW4(6468); |
| | | DCLRE1A(9937); C10orf46(143384); TIAL1(7073); F2(2147); INCENP(3619); |
| | | LGALS12(85329); DPF2(5977); CCND1(595); BIRC3(330); BIRC2(329); |
| | | CD3E(916); MLL(4297); EI24(9538); HRAS(3265); IGF2(3481); WT1 |
| | | (7490); CD59(966); FZD4(8322); CASP1(834); PPP2R1B(5519); IL18(3606); |
| | | CCND2(894); LTBR(4055); CNAP1(9918); PTPN6(5777); PEX5(5830); BCL2L14 |
| | | (79370); ESPL1(9700); PDE1B(5153); APAF1(317); GAS2L3(283431); |
| | | KNTC1(9735); RAN(5901); KRAS(3845); LALBA(3906); BTG1(694); |
| | | KCTD10(83892); DIABLO(56616); MPHOSPH9(10198); SCARB1(949); TNFRSF19 |
| | | (55504); BRCA2(675); FLJ22624(79866); CUL4A(8451); LATS2 |
| | | (26524); CSNK1A1L(122011); TXNDC(81542); PSEN1(5663); VASH1(22846); |
| | | TRAF3(7187); GZMH(2999); GZMB(3002); NFKBIA(4792); LTBP2 |
| | | (4053); MOAP1(64112); JAG2(3714); ZNF609(23060); CSK(1445); IGF1R |
| | | (3480); SLC12A6(9990); BMF(90427); DAPK2(23604); CSNK1G1(53944); |
| | | RAB40C(57799); DNAJA3(9093); CARD15(64127); CYLD(1540); CTCF(10664); |
| | | C16orf55(124045); CDK10(8558); AXIN1(8312); GNG13(51764); ERN2 |
| | | (10595); TRADD(8717); SLC12A4(6560); SMPD3(55512); PAFAH1B1 |
| | | (5048); NTN1(9423); MAPK7(5598); LIG3(3980); NGFR(4804); CROP(51747); |
| | | TLK2(11011); PRKCA(5578); CARD14(79092); MNT(4335); P2RX1 |
| | | (5023); MPP2(4355); HDAC5(10014); FLJ46026(400627); CABLES1(9176 |

Table 4-74

| | | |
|---|---|---|
| | | 8); MAPK4(5596); MALT1(10892); ESCO1(114799); BCL2(596); TXNL4A |
| | | (10907); CIRBP(1153); HMG20B(10362); CHAF1A(10036); ILF3(3609); DNM2 |
| | | (1785); PIK3R2(5296); APOE(348); NALP2(55655); AURKC(6795); ZNF443 |
| | | (10224); LOC163131(163131); TGFB1(7040); PPP1R13L(10848); |
| | | CGB7(94027); MCM8(84515); STK4(6789); CD40(958); CSE1L(1434); PARD6B |
| | | (84612); BCL2L1(598); RBL1(5933); CTCFL(140690); SYCP2(10388); |
| | | DIDO1(11083); SON(6651); PDXK(8566); BTG3(10950); HEMK2(29104); |
| | | SNF1LK(150094); ADORA2A(135); NF2(4771); TNRC6B(23112); EP300 |
| | | (2033); GTSE1(51512); MAPK8IP2(23542); BID(637); MAPK1(5594); PPM1F |
| | | (9647); PDGFB(5155); TOB2(10766); TAF1(6872); AGTR2(186); PNMA3 |
| | | (29944); PPP2R3B(28227); SMC1L1(8243); HCFC1(3054); MTCP1(4515); |
| hsa-miR-516-5p | 113; | TP73(7161); SESN2(83667); HDAC1 (3065); RBBP4(5928); UTP11L(51118); |
| | | MACF1(23499); CDC20(991); CDKN2C(1031); SH3GLB1(51100); CDC7 |
| | | (8317); PROK1(84432); BCL9(607); DAP3(7818); PPP1R12B(4660); FRAP1 |
| | | (2475); C1orf126(200197); CASP9(842); FAF1(11124); BCL10(8915); PYGO2 |
| | | (90780); CD84(8832); ASPM(259266); PPP1CB(5500); BIRC6(57448); |
| | | MSH2(4436); HK2(3099); CFLAR(8837); CASP10(843); CD28(940); ALS2CR19 |
| | | (117583); COL4A3(1285); PSMD1(5707); SEPT2(4735); CGREF1 |
| | | (10669); CARD12(58484); RTN4(57142); TIA1(7072); SEPT10(151011); BUB1 |
| | | (699); BIN1(274); ERCC3(2071); MCM6(4175); TLK1(9874); FRZB(2487); |
| | | STAT1(6772); CLK1(1195); BARD1(580); CUL3(8452); HDAC4(9759); |
| | | PCAF(8850); PDCD6IP(10015); NKTR(4820); APPL(26060); SMC4L1(10051); |
| | | TP73L(8626); PROK2(60675); GSK3B(2932); STAG1(10274); SIAH2 |
| | | (6478); SEPT11(55752); SPP1(6696); NFKB1(4790); FGF2(2247); PLK4 |
| | | (10733); GAK(2580); APBB2(323); FLJ38991(285521); BTC(685); PRKG2 |
| | | (5593); UNC5C(8633); LEF1(51176); RNF150(57484); NEK1(4750); SEP2 |
| | | (6502); CARD6(84674); APC(324); RAD50(10111); ERGIC1(57222); BIRC1 |
| | | (4671); MCC(4163); ATG12(9140); SEMA6A(57556); IRF1(3659); PPP2CA |
| | | (5515); CDC23(8697); CDC25C(995); NRG2(9542); HDAC3(8841); E2F3 |
| | | (1871); TNF(7124); AIF1(199); PARC(23113); HECA(51696); NEDD9(4739); |
| | | MDC1(9656); BAK1(578); CUL7(9820); MCM3(4172); DST(667); SESN1 |
| | | (27244); FRK(2444); MAP3K5(4217); PLAGL1(5325); LPAL2(80350); PDCD2 |
| | | (5134); RINT1(60561); CUL1(8454); MAD1L1(8379); TRIAD3(54476); |
| | | PMS2(5395); ELMO1(9844); CDK6(1021); MCM7(4176); BRAF(673); ESCO2 |
| | | (157570); UNC5D(137970); TACC1(6867); E2F5(1875); RIPK2(8767); |
| | | TNFRSF10B(8795); OPRK1(4986); CCNE2(9134); TP53INP1(94241); TNFRSF11B |
| | | (4982); MTSS1(9788); CDC37L1(55664); UNC13B(10497); TAF1L |
| | | (138474); SEMA4D(10507); PPP6C(5537); CIZ1(25792); TSC1(7248); |
| | | SIRT1(23411); SGPL1(8879); UNC5B(219699); FAS(355); FRAT1(10023); |
| | | ENTPD7(57089); LZTS2(84445); BTRC(8945); PDCD11(22984); TCF7L2 |
| | | (6934); DOCK1(1793); SFMBT2(57713); PARD3(56288); ZWINT(11130); |
| | | FRAT2(23401); FBXW4(6468); C10orf46(143384); MKI67(4288); STIM1 |
| | | (6786); WEE1(7465); GAS2(2620); API5(8539); EXT2(2132); INCENP(3619); |
| | | ATM(472); LOH11CR2A(4013); DKK3(27122); BAD(572); CDCA5(113130); |
| | | BRMS1(25855); SESN3(143686); CASP4(837); CASP5(838); IL18(3606); |
| | | CCND2(894); CNAP1(9918); PTPN6(5777); KIAA1467(57613); MSRB3 |
| | | (253827); SOCS2(8835); APAF1(317); TMEM132B(114795); CDCA3(83461); |
| | | BCAT1(586); LALBA(3906); DDIT3(1649); DIP13B(55198); ANAPC7 |
| | | (51434); HSMPP8(54737); BRCA2(675); ING1(3621); CDC16(8881); FOXO1A |
| | | (2308); BCL2L2(599); TRAF3(7187); ACIN1(22985); CIDEB(27141); |
| | | GZMB(3002); EGLN3(112399); CDKL1(8814); DLG7(9787); NEK9(91754); |
| | | BUB1B(701); ATP10A(57194); PDCD7(10081); KREMEN2(79412); DNASE1 |
| | | (1773); PAPD5(64282); ELMO3(79767); CDK10(8558); ERN2(10595); |
| | | MAPK3(5595); SEPT1(1731); DNAJA2(10294); FTS(64400); APPBP1 |
| | | (8883); SLC12A4(6560); TXNL4B(54957); HIC1(3090); NTN1(9423); MAPK7 |
| | | (5598); NF1(4763); PRKCA(5578); MNT(4335); NALP1(22861); TP53(7157); |
| | | GAS7(8522); SPAG5(10615); CABLES1(91768); MAPK4(5596); MALT1 |
| | | (10892); PHLPP(23239); SERPINB2(5055); BCL2(596); FZR1(51343); |
| | | UHRF1(29128); PIN1(5300); PIK3R2(5296); CCNE1(898); APLP1(333); |
| | | MAP3K10(4294); ARHGEF1(9138); FOSB(2354); DAPK3(1613); SLC25A23 |
| | | (79085); FCER2(2208); ERCC2(2068); CARD8(22900);KLK10(5655); TRIB3 |
| | | (57761); MCM8(84515); CEP250(11190); PARD6B(84612); MX1(4599); |
| | | CDC45L(8318); KREMEN1(83999); PGEA1(25776); EP300(2033); GTSE1 |
| | | (51512); MAPK8IP2(23542); MAPK1(5594); DMC1(11144); TOB2(10 |

Table 4-75

| | | |
|---|---|---|
| | | 766); MAPK12(6300); TAF1(6872); BIRC4(331); STAG2(10735); RP6-213H19.1 |
| | | (51765); FIGF(2277); SMC1L1(8243); SEPT6(23157); CUL4B(8450); |
| | | PDCD8(9131); ARHGEF6(9459); |
| hsa-miR-517* | 114; | NBL1(4681); SESN2(83667); KHDRBS1(10657); MACF1(23499); CDC7 |
| | | (8317); ADORA1(134); MDM4(4194); IL24(11009); CASP9(842); NOTCH2 |
| | | (4853); MCL1(4170); NMNAT2(23057); ASPM(259266); BIRC6(57448); HK2 |
| | | (3099); TCF7L1(83439); RIF1(55183); CFLAR(8837); CASP10(843); FZD7 |
| | | (8324); STK17B(9262); CUL3(8452); FANCD2(2177); ACPP(55); FXR1 |
| | | (8087); TP73L(8626); TADA3L(10474); TUSC4(10641); CCNG2(901); NFKB1 |
| | | (4790); PPBP(5473); CARD6(84674); ERBB2IP(55914); RAD17(5884); |
| | | APC(324); RAD50(10111); BNIP1(662); DAP(1611); PRLR(5618); BIRC1 |
| | | (4671); IRF1(3659); PPP2CA(5515); PFDN1(5201); BAG2(9532); PTP4A1 |
| | | (7803); HECA(51696); SASH1(23328); DHX16(8449); MDC1(9656); CCND3 |
| | | (896); CUL7(9820); DST(667); BCLAF1(9774); FOXK1(221937); HDAC9 |
| | | (9734); EGFR(1956); BCAP29(55973); TRIAD3(54476); SFRP4(6424); INHBA |
| | | (3624); HIP1(3092); CDK6(1021); MTMR9(66036); WISP1(8840); LZTS1 |
| | | (11178); TNFRSF10B(8795); ADRA1A(148); ANKRD15(23189); TSC1 |
| | | (7248); DKK1(22943); FAS(355); CSPG6(9126); C10orf46(143384); MKI67 |
| | | (4288); DPF2(5677); RPS6KB2(6199); BIRC2(329); CCDC15(80071); IGF2 |
| | | (3481); APBB1(322); MTL5(9633); SESN3(143686); ESPL1(9700); SOCS2 |
| | | (8835); NOL1(4839); BCAT1(586); SLC38A1(81539); ANAPC7(51434); |
| | | ANAPC5(51433); MPHOSPH9(10198); TNFRSF19(55504); BRCA2(675); |
| | | CUL4A(8451); FOXO1A(2308); LCP1(3936); BCL2L2(599); TXNDC(81542); |
| | | PSEN1(5663); BMF(90427); MYO5A(4644); CSNK1G1(53944); TRAF7 |
| | | (84231); TNFRSF12A(51330); CARD15(64127); CYLD(1540); AXIN1(8312); |
| | | TNFSF12(8742); TRAF4(9618); CDC6(990); MAPT(4137); SLC9A3R1 |
| | | (9368); ANAPC11(51529); TP53(7157); AURKB(9212); CABLES1(91768); |
| | | MALT1(10892); AXL(558); BCL2L12(83596); FUT1(2523); MCM8(84515); |
| | | PLCB1(23236); CEP250(11190); CTNNBL1(56259); STK4(6789); CTCFL |
| | | (140690); DIDO1(11083); SON(6651); BCL2L13(23786); GAS2L1(10634); |
| | | EP300(2033); EDA(1896); MLLT7(4303); PPP2R3B(28227); SMC1L1(8243); |
| hsa-miR-517a | 115; 116; | PRKCZ(5590); PHF13(148479); EFHD2(79180); CLIC4(25932); CCDC28B |
| hsa-miR-517c | | (79140); C1orf108(79647); MACF1(23499); ZYG11B(79699); CPT2(1376); |
| | | NFIA(4774); USP1(7398); TMEM56(148534); SMG7(9887); TNFRSF18 |
| | | (8784); SDF4(51150); CDC2L2(985); KIAA0495(57212); FRAP1(2475); EIF4G3 |
| | | (8672); WNT4(54361); LUZP1(7798); ZNF436(80818); IL22RA1(58985); |
| | | PAQR7(164091); FOXJ3(22887); SLC6A9(6536); JUN(3725); C1orf173 |
| | | (127254); OLFM3(118427); NPHS2(7827); DENND1B(163486); KIF21B |
| | | (23046); TNNI1(7135); PCNXL2(80003); IRF2BP2(359948); HNRPU(3192); |
| | | POLR1B(84172); RIF1(55183); PSMD1(5707); PDIA6(10130); RTN4(57142); |
| | | FLJ14397(84865); IL1B(3553); BIN1(274); LYPD1(116372); STK39 |
| | | (27347); STAT1(6772); GPR55(9290); NR1D2(9975); PDCD6IP(10015); MLH1 |
| | | (4292); BSN(8927); APPL(26060); IGSF4D(253559); ALCAM(214); SGEF |
| | | (26084); PPM1L(151742); SKIL(6498); OXTR(5021); SRGAP3(9901); |
| | | TMEM111(55831); HYAL1(3373); LRIG1(26018); RYBP(23429); ABI3BP |
| | | (25890); SEMA5B(54437); TMCC1(23023); MGC40579(256356); LOC348840 |
| | | (348840); SLIT2(9353); AREG(374); LARP2(55132); STOX2(56977); SLC26A1 |
| | | (10861); PPP2R2C(5522); AFAP(60312); LOC201895(201895); APBB2 |
| | | (323); PGDS(27306); LEF1(51176); TNIP3(79931); ENPP6(133121); |
| | | NIPBL(25836); ISL1(3670); ERBB2IP(55914); SERF1A(8293); CDC42SE2 |
| | | (56990); MFAP3(4238); SMA5(11042); FCHSD1(89848); SH3TC2(79628); |
| | | TNIP1(10318); DBN1(1627); MAPK9(5601); DKFZp686I15217(401232); |
| | | HFE(3077); HIST1H2AG(8969); EGFL8(80864); TTBK1(84630); SH3BGRL2 |
| | | (83699); GPR126(57211); TULP4(56995); RP11-145H9.1(340156); |
| | | GFOD1(54438); RNF39(80352); TMEM30A(55754); BACH2(60468); MCHR2 |
| | | (84539); STX7(8417); BCLAF1(9774); EPM2A(7957); AHR(196); NUPL2 |
| | | (11097); FLJ90586(135932); TRIAD3(54476); HOXA5(3202); CARD4 |
| | | (10392); SFRP4(6424); TNS3(64759); BRAF(673); TNKS(8658); PTK2B(2185); |
| | | ADAM9(8754); SULF1(23213); KCNS2(3788); COLEC10(10584); LGI3 |
| | | (203190); TRIM35(23087); KIF13B(23303); PLAT(5327); RDHE2(195814); |
| | | TRPA1(8989); JPH1(56704); ST3GAL1(6482); DAPK1(1612); STXBP1 |
| | | (6812); CDKN2A(1029); SMU1(55234); RFK(55312); TSCOT(57864); TRAF1 |
| | | (7185); DENND1A(57706); PPP6C(5537); PTGES(9536); AKR1C4 |
| | | (1109); MGC16291(84856); LRRTM3(347731); DOCK1(1793); C10orf39(2 |

Table 4-76

| | | |
|---|---|---|
| | | 82973); LRRC27(80313); FRMD4A(55691); RSU1(6251); MARCH8(220972); |
| | | C10orf54(64115); C11orf36(283303); MADD(8567); MS4A4A(51338); |
| | | PACS1(55690); NPAS4(266743); CUL5(8065); ARCN1(372); STS-1(84959); |
| | | CDKN1C(1028); EIF4G2(1982); C11orf55(399879); KCTD14(65987); FLJ38159 |
| | | (220388); KRTHB6(3892); PFDN5(5204); LEMD3(23592); THAP2 |
| | | (83591); LOC338809(338809); SUDS3(64426); FLJ38663(91574); FZD10 |
| | | (11211); TNFRSF1A(7132); LRP6(4040); BHLHB3(79365); C12orf22(81566); |
| | | IGF1(3479); C12orf49(79794); MGC7036(196383); CHFR(55743); CUL4A |
| | | (8451); SLITRK1(114798); SMOC1(64093); C14orf130(55148); BDKRB2 |
| | | (624); CCNK(8812); TRAF3(7187); KIAA0125(9834); PRMT5(10419); EGLN3 |
| | | (112399); KCNH5(27133); FLJ39779(400223); SYNJ2BP(55333); SPTLC2 |
| | | (9517); CHES1(1112); LOC440248(440248); BUB1B(701); RNF111 |
| | | (54778); APH1B(83464); SMAD3(4088); KIAA1199(57214); ALPK3(57538); |
| | | PRTG(283659); SLTM(79811); DAPK2(23604); SEC11L1(23478); TARSL2 |
| | | (123283); E4F1(1877); PDPK1(5170); SPN(6693); TP53TG3(24150); |
| | | NKD1(85407); CYLD(1540); PSKH1(5681); SPG7(6687); LOC339123(339123); |
| | | CTRL(1506); SLC12A4(6560); MGC26885(124044); HIC1(3090); ALOX12 |
| | | (239); C17orf49(124944); RAB11FIP4(84440); RAPGEFL1(51195); |
| | | XYLT2(64132); TLK2(11011); PRKCA(5578); CACNG4(27092); KCNJ2(3759); |
| | | CDK3(1018); SPHK1(8877); BAIAP2(10458); HEXDC(284004); VAMP2 |
| | | (6844); GAS7(8522); MYH4(4622); FLJ31196(146802); TCF2(6928); |
| | | KRT9(3857); MAP3K14(9020); SFRS1(6426); MPO(4353); LIMD2(80774); |
| | | CCDC47(57003); CCDC46(201134); HELZ(9931); C17orf62(79415); WDR45L |
| | | (56270); B3GNTL1(146712); CTDP1(9150); MPPE1(65258); ANKRD29 |
| | | (147463); ZNF521(25925); DYM(54808); CBLN2(147381); NCLN(56926); |
| | | TNFAIP8L1(126282); MRPL4(51073); LDLR(3949); UBA52(7311); GRAMD1A |
| | | (57655); FFAR2(2867); AXL(558); ARHGEF1(9138); PRMT1(3276); |
| | | FCAR(2204); NALP2(55655); FLJ45684(400666); GNG7(2788); DAPK3 |
| | | (1613); ZBTB7A(51341); CACNA1A(773); HAPLN4(404037); SF4(57794); |
| | | NALP7(199713); ATRN(8455); CDC25B(994); CBFA2T2(9139); C20orf118 |
| | | (140711); C20orf77(58490); STK4(6789); C20orf11(54994); ZGPAT(84619); |
| | | CTCFL(140690); CTSZ(1522); JAM2(58494); KIAA0179(23076); ITGB2 |
| | | (3689); SEPT5(5413); ADRBK2(157); EP300(2033); HTF9C(27037); IGLL1 |
| | | (3543); CBX6(23466); C22orf9(23313); RP1-29C18.7(348645); MAPK12 |
| | | (6300); CLCN4(1183); PCTK1(5127); OATL1(4943); PLP2(5355); GPR64 |
| | | (10149); SMC1L1(8243); AMMECR1(9949); SEPT6(23157); IRAK1(3654); |
| hsa-miR-517b | 117; | MACF1(23499); CDC20(991); NFIA(4774); TRAF5(7188); CIAS1(114548); |
| | | OXCT2(64064); BIRC6(57448); HK2(3099); RIF1(55183); NRP2(8828); PSMD1 |
| | | (5707); AFF3(3899); BIN1(274); HDAC4(9759); KNG1(3827); UBE1C |
| | | (9039); SIAH2(6478); FGFRL1(53834); GAB1(2549); SLC25A4(291); GAK |
| | | (2580); AFAP(60312); APBB2(323); LEF1(51176); NEK1(4750); SKP2 |
| | | (6502); NIPBL(25836); SERF1A(8293); UNC5A(90249); RNF130(55819); |
| | | DKFZp686I15217(401232); RIPK1(8737); TTK(7272); DST(667); BCLAF1 |
| | | (9774); AHR(196); HBP1(26959); CASP2(835); PMS2(5395); TNFRSF11B |
| | | (4982); RECK(8434); ABL1(25); COL5A1(1289); TRAF1(7185); TSC1(7248); |
| | | CASC2(255082); DOCK1(1793); DLG5(9231); CUL5(8065); LEMD3(23592); |
| | | CDK2AP1(8099); F7(2155); TRAF7(84231); CTCF(10664); STX1B2 |
| | | (112755); SLC12A4(6560); SMPD3(55512); HIC1(3090); PAFAH1B1(5048); |
| | | CDK3(1018); SPHK1(8877); ANAPC11(51529); TIAF1(9220); CSNK1D |
| | | (1453); MAPRE2(10982); ESCO1(114799); AURKC(6795); AES(166); CDC25B |
| | | (994); PHF20(51230); CTNNBL1(56259); SEPT5(5413); PPM1F(9647); |
| | | MN1(4330); DMC1(11144); SMC1L1(8243); SEPT6(23157); |
| hsa-miR-518b | 118; 119; 120; | PRKCZ(5590); DAP3(7818); TGFB2(7042); CCNL2(81669); ACOT7(11332); |
| hsa-miR-518c | 926; | CTNNBIP1(56998); FRAP1(2475); DEDD(9191); TP53BP2(7159); BRRN1 |
| hsa-miR-518d | | (23397); ANAPC1(64682); ERCC3(2071); IFIH1(64135); CUL3(8452); |
| | | RBM5(10181); OPA1(4976); PRICKLE2(166336); WHSC1(7468); CD38 |
| | | (952); HCAP-G(64151); IL8(3576); APBB2(323); CCNI(10983); PRKG2(5593); |
| | | NIPBL(25836); DAP(1611); PPP2CA(5515); CDC23(8697); SH3PXD2B |
| | | (285590); RCLAF1(9774); PMS2L5(5383); STAG3(10734); ELMO1(9844); |
| | | SFRP4(6424); WISP1(8840); TNFRSF10D(8793); TP53INP1(94241);CDKN2B |
| | | (1030); PTCH(5727); ANAPC2(29882); CSPG6(9126); TCF7L2(6934); |
| | | FRAT2(23401); DCLRE1A(9937); SSSCA1(10534); LOH11CR2A(4013); |
| | | TOLLIP(54472); CDKN1C(1028); WT1(7490); FGF4(2249); FGF3(2248); |
| | | SHANK2(22941); PAK1(5058); BCL9L(283149); CDKN1B(1027); TEG |

Table 4-77

| | | |
|---|---|---|
| | | T(7009); ACVR1B(91); KIAA0152(9761); TNFRSF1A(7132); ABCB9(23457); |
| | | HSMPP8(54737); MCF2L(23263); CSNK1A1L(122011); SOCS4(122809); |
| | | TRAF3(7187); DAPK2(23604); CTCF(10664); FLJ12998(64779); GSG2 |
| | | (83903); FZD2(2535); BIRC5(332); GAS2L2(246176); MAPK4(5596); CHAF1A |
| | | (10036); UBA52(7311); BCL3(602); FOSB(2354); PPP2R1A(5518); ERCC2 |
| | | (2068); STK4(6789); VAPB(9217); CABLES2(81928); BTG3(10950); |
| | | ITGB2(3689); MAPK8IP2(23542); MAPK1(5594); MN1(4330); PDGFB(5155); |
| | | IKBKG(8517); SMC1L1(8243); |
| hsa-miR-518c* | 121; | DFFB(1677); H6PD(9563); NBL1(4681); CDC42(998); PPP1R8(5511); KHDRBS1 |
| | | (10657); LCK(3932); MACF1(23499); NFYC(4802); CDKN2C(1031); |
| | | CDC7(8317); CDC14A(8556); SYCP1(6847); BCL9(607); PEA15(8682); |
| | | PPP1R12B(4660); ADORA1(134); IL24(11009); CCNL2(81669); CDC2L2 |
| | | (985); TNFRSF9(3604); DFFA(1676); FRAP1(2475); RCC2(55920); E2F2(1870); |
| | | CLSPN(63967); TESK2(10420); GFI1(2672); NRAS(4893); PYGO2(90780); |
| | | GLRX2(51022); TP53BP2(7159); MAPRE3(22924); PPP1CB(5500); |
| | | BIRC6(57448); ANTXR1(84168); HK2(3099); MAL(4118); BRRN1(23397); |
| | | BCL2L11(10018); TAIP-2(80034); ZAK(51776); CASP10(843); CASP8(841); |
| | | CYP20A1(57404); ALS2CR19(117583); COL4A3(1285); PSMD1(5707); |
| | | FLJ45964(401040); YWHAQ(10971); E2F6(1876); PPM1G(5496); EIF2AK2 |
| | | (5610); TIA1(7072); SEPT10(151011); BIN1(274); CUL3(8452); HDAC4 |
| | | (9759); FANCD2(2177); MLH1(4292); ZNF621(285268); LIMD1(8994); |
| | | RBM5(10181); GNL3(26354); APPL(26060); PCNP(57092); SENP2(59343); |
| | | TP73L(8626); RAF1(5894); TRAIP(10293); ADCY5(111); PTPLB(201562); |
| | | PIK3CB(5291); HD(3064); HCAP-G(64151); ANAPC4(29945); EREG |
| | | (2069); CCNG2(901); FGF2(2247); AFAP(60312); SFRP2(6423); NEK1(4750); |
| | | KIAA1909(153478); PDCD6(10016); CARD6(84674); ERBB2IP(55914); |
| | | TCF7(6932); KIAA0141(9812); DAP(1611); PRLR(5618); GDNF(2668); BIRC1 |
| | | (4671); MCC(4163); SEMA6A(57556); CDC23(8697); CD14(929); FGF1 |
| | | (2246); CSNK2B(1460); MAPK13(5603); DAAM2(23500); PARC(23113); |
| | | VEGF(7422); CDC5L(988); IL17(3605); TTK(7272); FOXO3A(2309); TCF21 |
| | | (6943); NEDD9(4739); DHX16(8449); MDC1(9656); IER3(8870); DAXX |
| | | (1616); MCM3(4172); DST(667); ATG5(9474); SGK(6446); MAP3K5(4217); |
| | | KATNA1(11104); FOXK1(221937); SEPT7(989); EGFR(1956); FZD1(8321); |
| | | BCAP29(55973); ARHGEF5(7984); RBM33(155435); TRIAD3(54476); |
| | | PMS2(5395); PSCD3(9265); INHBA(3624); CDK6(1021); TNFRSF10C |
| | | (8794); BNIP3L(665); UNC5D(137970); TACC1(6887); SULF1(23213); RIPK2 |
| | | (8767); WISP1(8840); PINX1(54984); LZTS1(11178); TNFRSF10A(8797); |
| | | ADRA1A(148); TRIM35(23087); SFRP1(6422); TP53INP1(94241); RAD21 |
| | | (5885); EXT1(2131); MTSS1(9788); UNC13B(10497); NTRK2(4915); |
| | | DAPK1(1612); C9orf47(286223); INVS(27130); PALM2-AKAP2(445815); |
| | | PTGS1(5742); ABL1(25); CDKN2A(1029); BAG1(573); BICD2(23299); TRAF1 |
| | | (7185); CIZ1(25792); TSC1(7248); CARD9(64170); ZMYND11(10771); |
| | | SIRT1(23411); PTEN(5728); BTRC(8945); SUFU(51684); PDCD11(22984); |
| | | TCF7L2(6934); GPR26(2849); LRRC27(80313); PARD3(56288); LRRC20 |
| | | (55222); FRAT2(23401); TIAL1(7073); STIM1(6786); WEE1(7465); HIPK3 |
| | | (10114); EXT2(2132); TSPAN18(90139); MDK(4192); C11orf49(79096); |
| | | VEGFB(7423); DPF2(5977); SSSCA1(10534); RPS6KB2(6199); CCND1 |
| | | (595); BIRC3(330); ATM(472); FLJ45300(399957); HRAS(3265); RHOG |
| | | (391); DKK3(27122); CD59(966); BRMS1(25855); FGF4(2249); FGF3(2248); |
| | | DLG2(1740); FZD4(8322); SESN3(143686); PPP2R1B(5519); ZW10(9183); |
| | | BCL9L(283149); CCND2(894); TNFRSF7(939); PTMS(5763); FLJ20489 |
| | | (55652); PA2G4(5036); MDM2(4193); CRADD(8738); GAS2L3(283431); |
| | | CACNA2D4(93589); HDAC7A(51564); MCRS1(10445); FMNL3(91010); FAIM2 |
| | | (23017); KIAA0286(23306); CDK4(1019); WIF1(11197); PCTK2(5128); |
| | | HRK(8739); CHFR(55743); HSMPP8(54737); RB1(5925); RFP2(10206); |
| | | SUGT1(10910); FLJ40296(122183); LATS2(26524); FOXO1A(2308); NEK3 |
| | | (4752); BCL2L2(599); RNF31(55072); PNN(5411); TRAF3(7187); EGLN3 |
| | | (112399); LTBP2(4053); NEK9(91754); AKT1(207); FGF7(2252); KIF23 |
| | | (9493); FLJ25222(374666); BMF(90427); TTBK2(146057); PYGO1(26108); |
| | | CSNK1G1(53944); PDCD7(10081); BCL2A1(597); DKFZp434I1020(196968); |
| | | CCNF(899); DNAJA3(9093); CYLD(1540); RBL2(5934); E2F4(1874); |
| | | NFATC3(4775); PKMYT1(9088); GSPT1(2935); ERN2(10595); LOC440350 |
| | | (440350); PYCARD(29108); RANBP10(57610); SLC12A4(6560); SMPD3 |
| | | (55512); HIC1(3090); PAFAH1B1(5048); TNFSF12(8742); ALOX15B |
| | | (247); MAPK7(5598); NF1(4763); MAPT(4137); NGFR(4804); PPM1D(849 |

Table 4-78

| | | |
|---|---|---|
| | | 3); PRKCA(5578); KPNA2(3838); MAP2K6(5608); CARD14(79092); ZZEF1 |
| | | (23140); GAS7(8522); GRAP(10750); SPAG5(10615); TIAF1(9220); FLJ31952 |
| | | (146857); GAS2L2(246176); BECN1(8678); HDAC5(10014); RNF43 |
| | | (54894); SEPT4(5414); ERN1(2081); CABLES1(91768); MAPK4(5596); DCC |
| | | (1630); SERPINB2(5055); BCL2(596); FZR1(51343); DNM2(1785); CCNE1 |
| | | (898); AXL(558); ARHGEF1(9138); APOE(348); FOSB(2354); SPHK2(56848); |
| | | ATF5(22809); NALP2(55655); AURKC(6795); LOC390980(390980); |
| | | ELAVL1(1994); DNASE2(1777); PRX(57716); DEDD2(162989); ERCC2 |
| | | (2068); PPP1R13L(10848); CARD8(22900); KLK10(5655); PLCB1(23236); |
| | | CEP250(11190); CTNNBL1(56259); MYBL2(4605); KCNK15(60598); STK4 |
| | | (6789); UBE2C(11065); CSE1L(1434); CSNK2A1(1457); ZNF343(79175); |
| | | E2F1(1869); RP5-1054A22.3(85449); PTPRT(11122); AURKA(6790); CABLES2 |
| | | (81928); DIDO1(11083); SON(6651); MX1(4599); SNF1LK(150094); |
| | | CDC45L(8318); GAS2L1(10634); NF2(4771); EP300(2033); BIK(638); |
| | | GTSE1(51512); DGCR2(9993); MAPK1(5594); PPM1F(9647); CARD10(29775); |
| | | SMC1L2(27127); CHKB(1120); PCTK1(5127); MAGEH1(28986); TAF1 |
| | | (6872); STAG2(10735); CD40LG(959); SPRY3(10251); FIGF(2277); HCFC1 |
| | | (3054); MECP2(4204); |
| hsa-miR-518e | 122; 123; | PRKCZ(5590); FAM79A(127262); KCNAB2(8514); C1orf188(148646); TAS1R1 |
| hsa-mir-518f | | (80835); H6PD(9563); EFHD2(79180); C1orf151(440574); PTPRU(10076); |
| | | C1orf83(127428); CYR61(3491); CDC14A(8556); DAP3(7818); ATF6 |
| | | (22926); MR1(3140); TROVE2(6738); NAV1(89796); TGFB2(7042); C1orf95 |
| | | (375057); CABC1(56997); C1orf69(200205); EDARADD(128178); CCNL2 |
| | | (81669); CDC2L2(985); PEX10(5192); ACOT7(11332); PLEKHG5(57449); |
| | | FRAP1(2475); C1orf89(79383); AKR7A2(8574); FAF1(11124); WDR78 |
| | | (79819); CSDE1(7812); C1orf43(25912); FCRL4(83417); FCRL2(79368); DEDD |
| | | (9191); GPR161(23432); RIPK5(25778); TP53BP2(7159); PARP1(142); |
| | | FLJ45717(388759); FLJ41046(400940); GREB1(9687); CAD(790); YIPF4 |
| | | (84272); SOCS5(9655); FLJ12788(64427); BRRN1(23397); NPAS2(4862); |
| | | GORASP2(26003); FLJ32063(150538); FZD7(8324); ALS2CR19(117583); |
| | | HRB(3267); ALPI(248); TRAF3IP1(26146); OACT2(129642); FLJ21945 |
| | | (80304); DNMT3A(1788); NRXN1(9378); TGOLN2(10618); STARD7(56910); |
| | | ANAPC1(64682); FLJ10996(54520); ERCC3(2071); MGC12981(84317); |
| | | GTDC1(79712); BARD1(580); EDEM1(9695); C3orf42(84657); PCAF |
| | | (8850); EIF1B(10289); RBM5(10181); FAM62C(83850); VGLL4(9686); RAF1 |
| | | (5894); AXUD1(64651); ZNF445(353274); MAP4(4134); GMPPB(29925); |
| | | TMEM110(375346); FHIT(2272); PRICKLE2(166336); MUC13(56667); |
| | | C3orf27(23434); SERPINI2(5276); EIF5A2(56648); WHSC1(7468); EVC |
| | | (2121); CD38(952); ZAR1(326340); THAP6(152815); CDS1(1040); HADHSC |
| | | (3033); TLR2(7097); WWC2(80014); LRPAP1(4043); WDR1(9948); MIST |
| | | (116449); APBB2(323); PRKG2(5593); BBS7(55212); NRSC2(4306); NIPBL |
| | | (25836); PIK3R1(5295); MARVELD2(153562); DCP2(167227); CSNK1G3 |
| | | (1456); PHF15(23338); PCDHGA1(56114); FOXI1(2299); HMP19(51617); |
| | | BTNL9(153579); CRR9(81037); FKSG14(64105); ZBED3(84327); OTP |
| | | (23440); DP58(340120); DHFR(1719); SLCO4C1(353189); FLJ20125(54826); |
| | | ZNF608(57507); FCHSD1(89848); SPRY4(81848); C1QTNF2(114898); |
| | | LOC221711(221711); HIST1H3E(8353); ZNF192(7745); C6orf134(79969); |
| | | ALF1(199); C6orf141(135398); TTK(7272); FOXO3A(2309); SLC35F1 |
| | | (222553); PLEKHG1(57480); SNX9(51429); QKI(9444); NOTCH4(4855); |
| | | C6orf125(84300); USP49(25862); COL9A1(1297); PDSS2(57107); STX7 |
| | | (8417); MGC35308(285800); AUTS2(26053); PMS2L5(5383); UPK3B(80761); |
| | | PSMC2(5701); LEP(3952); ZNF282(8427); EN2(2020); RBM33(155435); |
| | | LOC90639(90639); KIAA0644(9865); VPS41(27072); H2AFV(94239); POLR2J2 |
| | | (246721); FLJ31818(154743); ZC3HC1(51530); ABCF2(10061); TNFRSF10C |
| | | (8794); BAG4(9530); IKBKB(3551); FLJ45248(401472); WISP1 |
| | | (8840); EGR3(1960); TNFRSF10B(8795); CCDC25(55246); RAB11FIP1 |
| | | (80223); WHSC1L1(54904); RRAGA(10670); NTRK2(4915); PRDM12(59335); |
| | | ABL1(25); NUP214(8021); CDKN2A(1029); CCL21(6366); TMEM2 |
| | | (23670); PTCH(5727); TRIM14(9830); POLE3(54107); RAB14(51552); C9orf90 |
| | | (203245); C9orf140(89958); C10orf47(254427); PPYR1(5540); C10orf22 |
| | | (84890); LOC439985(439985); KIAA1128(54462); ZRANB1(54764); MTG1 |
| | | (92170); ITIH5(80760); FZD8(8325); ARHGAP22(58504); DUSP13(51207); |
| | | SEC31L2(25956); DCLRE1A(9937); SYT8(90019); MYOD1(4654); |
| | | MDK(4192); CD5(921); SIPA1(6494); RBM14(10432); YAP1(10413); NNMT |
| | | (4837); TAGLN(6876); FLJ45300(399957); SLC37A2(219855); C11orf37 |

Table 4-79

| | | |
|---|---|---|
| | | (440072); BET1L(51272); MUCDHL(53841); TOLLIP(54472); LOC221091 |
| | | (221091); DKFZp761E198(91056); FGF3(2248); PGM2L1(283209); FXYD2 |
| | | (486); BCL9L(283149); hCAP-D3(23310); TPI1(7167); LRRC23(10233); |
| | | ACVR1B(91); NR4A1(3164); HOXC5(3222); NXPH4(11247); DYRK2(8445); |
| | | RAP1B(5908); CRADD(8738); LOC196463(196463); FBXW8(26259); P2RX7 |
| | | (5027); BRI3BP(140707); FZD10(11211); SFRS8(6433); PARP11(57097); |
| | | ADCY6(112); ZNF385(25946); GLS2(27165); DDIT3(1649); C12orf49 |
| | | (79794); OASL(8638); CAMKK2(10645); RHOF(54509); ABCB9(23457); |
| | | RIMBP2(23504); HSMPP8(54737); SLC25A15(10166); AKAP11(11215); ING1 |
| | | (3621); MCF2L(23263); LAMP1(3916); TMCO3(55002); GAS6(2621); |
| | | FOXO1A(2308); FLJ13639(79758); EFNB2(1948); LOC440157(440157); |
| | | EIF2S1(1965); PAPLN(89932); C14orf115(55237); VASH1(22846); MAP3K9 |
| | | (4293); PNMA1(9240); TGFB3(7043); PTPN21(11099); PPP1R13B(23368); |
| | | FLJ39531(400360); CAPN3(825); KIAA1794(55215); C15orf42(90381); |
| | | LRRK1(79705); CCPG1(9236); PRTG(283659); SLTM(79811); CPEB1 |
| | | (64506); TSC2(7249); PDPK1(5170); KCTD5(54442); UBN1(29855); SNN |
| | | (8303); FUS(2521); TGFB1I1(7041); FLJ21736(79984); ELMO3(79767); CTCF |
| | | (10664); WWP2(11060); CDT1(81620); NME3(4832); PRSS22(64063); |
| | | GSPT1(2935); C16orf63(123811); TBC1D10B(26000); C16orf58(64755); |
| | | CMTM4(146223); MAF(4094); FLJ12998(64779); MVD(4597); MGC26885 |
| | | (124044); FOXN1(8456); HSD17B1(3292); FZD2(2535); MSI2(124540); |
| | | ARMC7(79637); SLC13A5(284111); TP53(7157); POLDIP2(26073); CORO6 |
| | | (84940); RAD51L3(5892); TMEM101(84336); DLX3(1747); MPO(4353); |
| | | PTRH2(51651); CSH2(1443); KIAA0427(9811); MAPK4(5596); ZNF521 |
| | | (25925); C18orf10(25941); FZR1(51343); CHAF1A(10036); CLEC4M(10332); |
| | | ICAM1(3383); ILF3(3609); GTPBP3(84705); PIK3R2(5296); UBA52(7311); |
| | | ACTN4(81); ZFP36(7538); MAP3K10(4294); MGC20255(90324); FOSB |
| | | (2354); SPHK2(56848); PRMT1(3276); FLJ16231(388561); RPS9(6203); |
| | | NALP2(55655); ZNF471(57573); DAPK3(1613); ZBTB7A(51341); TNFSF7 |
| | | (970); CTXN1(404217); ELAVL1(1994); MGC71805(403113); U2AF1L4 |
| | | (79713); PAF1(54623); FLJ41131(284325); CABP5(56344); ATPBD3(90353); |
| | | STK35(140901); CTNNBL1(56259); SLC32A1(140679); PTPN1(5770); |
| | | RAE1(8480); VAPB(9217); C20orf177(63939); SS18L1(26039); RTEL1(51750); |
| | | DNAJC5(80331); CSNK2A1(1457); SCRT2(85508); RSPO4(343637); |
| | | TASP1(55617); E2F1(1869); GGTL3(2686); RIMS4(140730); SLC13A3(64849); |
| | | SPATA2(9825); DIDO1(11083); SOX18(54345); CXADR(1525); C21orf63 |
| | | (59271); SON(6651); C21orf25(25966); TFF3(7033); C21orf2(755); PTTG1IP |
| | | (754); DGCR6(8214); SEC14L2(23541); TCN2(6948); MCM5(4174); |
| | | PGEA1(25776); CTA-216E10.6(79640); ADM2(79924); MAPK8IP2(23542); |
| | | DGCR14(8220); DGCR6L(85359); RP5-1119A7.4(80020); FLJ90680 |
| | | (400926); CBX7(23492); MLC1(23209); LOC440836(440836); CPT1B(1375); |
| | | ZC3H12B(340554); RP11-308D16.4(441522); ZNF185(7739); SMC1L1 |
| | | (8243); RP11-130N24.1(340533); NXF5(55998); |
| hsa-miR-520h | 124; 927; | TP73(7161); TNFRSF1B(7133); NUDC(10726); KHDRBS1(10657); RBBP4 |
| | | (5928); MACF1(23499); PLK3(1263); SH3GLB1(51100); CDC14A(8556); |
| | | BCL9(607); BNIPL(149428); CKS1B(1163); DAP3(7818); FASLG(356); RGS2 |
| | | (5997); MDM4(4194); IL24(11009); TGFB2(7042); TNFRSF9(3604); DFFA |
| | | (1676); FRAP1(2475); CLSPN(63967); FAF1(11124); GFI1(2672); SASS6 |
| | | (163786); DBT(1629); NOTCH2(4853); CTSS(1520); PYGO2(90780); CD5L |
| | | (922); DEDD(9191); GLRX2(51022); ASPM(259266); IL10(3586); BIRC6 |
| | | (57448); KCNIP3(30818); BRRN1(23397); BCL2L11(10018); RIF1(55183); |
| | | ZAK(51776); CFLAR(8837); CASP10(843); CASP8(841); CD28(940); TIA1 |
| | | (7072); MGAT4A(11320); RGPD5(84220); CUL3(8452); HDAC4(9759); |
| | | FANCD2(2177); VHL(7428); PDCD6IP(10015); PCNP(57092); FXR1(8087); |
| | | PSMD2(5708); ZFYVE20(64145); SEC22L3(9117); NME6(10201); PRKAR2A |
| | | (5576); MGC40579(256356); ATR(545); CD38(952); EREG(2069); THAP6 |
| | | (152815); SEPT11(55752); CCNG2(901); SPP1(6696); FGF2(2247); |
| | | GAK(2580); APBB2(323); LEF1(51176); CASP6(839); NIPBL(25836); ERBB2IP |
| | | (55914); CCNB1(891); RAD17(5884); F2R(2149); APC(324); TCF7 |
| | | (6932); G3BP(10146); SEMA5A(9037); PRLR(5618); PRKAA1(5562); BIRC1 |
| | | (4671); ATG12(9140); SEMA6A(57556); IRF1(3659); PPP2CA(5515); FBXW11 |
| | | (23291); TNF(7124); MAPK13(5603); CDKN1A(1026); PARC(23113); |
| | | VEGF(7422); PTP4A1(7803); HECA(51696); NEDD9(4739); ATXN1(6310); |
| | | DHX16(8449); NUDT3(11165); CUL7(9820); TNFRSF21(27242); MCM3 |
| | | (4172); DST(667); FRK(2444); SGK(6446); LATS1(9113); PDCD2(5134); |

Table 4-80

| | | |
|---|---|---|
| | | STK17A(9263); EGFR(1956); BCAP29(55973); CUL1(8454); TRIAD3(54476); |
| | | PMS2(5395); CYCS(54205); ELMO1(9844); TMED4(222068); CDK6 |
| | | (1021); MCM7(4176); FLJ13576(64418); PTN(5764); FASTK(10922); PIWIL2 |
| | | (55124); TNFRSF10C(8794); BNIP3L(665); PTK2B(2185); ESCO2(157570); |
| | | SPFH2(11160); TACC1(6867); UBE2V2(7336); SULF1(23213); FZD6 |
| | | (8323); MYC(4609); TNFRSF10D(8793); TNFRSF10A(8797); DUSP4(1846); |
| | | SFRP1(6422); DKK4(27121); TP53INP1(94241); EXT1(2131); ANKRD15 |
| | | (23189); JAK2(3717); UNC13B(10497); DAPK1(1612); C9orf47(286223); |
| | | INVS(27130); SLC31A1(1317); CDKN2B(1030); BAG1(573); CTNNAL1 |
| | | (8727); TNFSF15(9966); PPP6C(5537); CIZ1(25792); TSC1(7248); FAS |
| | | (355); KIF11(3832); FRAT1(10023); SUFU(51684); PDCD11(22984); FRAT2 |
| | | (23401); FBXW4(6468); DCLRE1A(9937); C10orf46(143384); MKI67(4288); |
| | | BNIP3(664); WEE1(7465); HIPK3(10114); KIAA1377(57562); ATM(472); |
| | | LOH11CR2A(4013); EI24(9538); EIF4G2(1982); WT1(7490); HRASLS3 |
| | | (11145); CLCF1(23529); FZD4(8322); BCL9L(283149); CCND2(894); CNAP1 |
| | | (9918); IAPP(3375); FLJ13236(79962); ESPL1(9700); GAS2L3(283431); |
| | | KNTC1(9735); GTF2H3(2967); FZD10(11211); LRP6(4040); BCAT1 |
| | | (586); HDAC7A(51564); LALBA(3906); FAIM2(23017); CDK4(1019); PAWR |
| | | (5074); BTG1(694); PCTK2(5128); HSMPP8(54737); TNFRSF19(55504); |
| | | BRCA2(675); RB1(5925); RFP2(10206); TFDP1(7027); FOXO1A(2308); NEK3 |
| | | (4752); CGRRF1(10668); C14orf111(51077); TRAF3(7187); DLG7(9787); |
| | | ERH(2079); LTBP2(4053); PGF(5228); NEK9(91754); SPTLC2(9517); |
| | | AKT1(207); MAPK6(5597); ABHD2(11057); SLC12A6(9990); BNIP2(663); |
| | | DAPK2(23604); PDCD7(10081); DNASE1(1773); SPN(6693); CARD15(64127); |
| | | CYB5B(80777); CDT1(81620); AXIN1(8312); SLC12A4(6560); PAFAH1B1 |
| | | (5048); TNFSF12(8742); ALOX15B(247); NLK(51701); MAPT(4137); |
| | | CROP(51747); PRR11(55771); TLK2(11011); SSTR2(6752); BIRC5(332); |
| | | CARD14(79092); P2RX1(5023); NALP1(22861); TP53(7157); GAS7(8522); |
| | | BRCA1(672); ERN1(2081); PHLPP(23239); BCL2(596); TXNL4A(10907); |
| | | GADD45B(4616); CHAF1A(10036); TNFAIP8L1(126282); DNM2(1765); |
| | | PIK3R2(5296); UBA52(7311); GRLF1(2909); ATF5(22809); SPIB(6689); |
| | | PPP2R1A(5518); NALP2(55655); DAPK3(1613); TNFSF14(8740); CDC37 |
| | | (11140); DNASE2(1777); PPP1R13L(10848); KLK10(5655); ZFPL(162967); |
| | | TRIB3(57761); PLCB1(23236); MAPRE1(22919); C20orf121(79183); |
| | | E2F1(1869); RBL1(5933); DIDO1(11083); SON(6651); APP(351); SNF1LK |
| | | (150094); NF2(4771); MCM5(4174); TNRC6B(23112); LOC63929(63929); |
| | | SEPT3(55964); PPARA(5465); MAPK8IP2(23542); MAPK1(5594); MN1 |
| | | (4330); DMC1(11144); MLLT7(4303); BIRC4(331); SEPT6(23157); |
| hsa-miR-523 | 125; | TARDBP(23435); EXTL1(2134); CTPS(1503); FAM46C(54855); BNIPL(149428); |
| | | IER5(51278); ARF1(375); PEX10(5192); C1orf126(200197); RCC2 |
| | | (55920); IL22RA1(58985); KIAA0319L(79932); TP53BP2(7159); OTX1(5013); |
| | | TCF7L1(83439); TEKT4(150483); TBR1(10716); BBS5(129880); KCNE4 |
| | | (23704); MGC42174(129563); CYP26B1(56603); PER2(8864); VHL(7428); |
| | | RAF1(5894); HD(3064); ZNF509(166793); SEC24B(10427); STX18(53407); |
| | | PPARGC1A(10891); MAD2L1(4085); IRX1(79192); CCNB1(891); CTNND2 |
| | | (1501); SMA4(11039); SMA5(11042); CD14(929); ANXA6(309); DBN1 |
| | | (1627); DDX41(51428); C6orf134(79969); GUSBL1(387036); TULP1(7287); |
| | | CUL7(9820); GUSBL2(375513); BCLAF1(9774); POM121(9883); POR |
| | | (5447); LOC155060(155060); MGC33584(285971); URG4(55665); TMED4 |
| | | (222068); WBSCR16(81554); CDK6(1021); ADCK5(203054); PGM5(5239); |
| | | CDK9(1025); GRIN1(2902); LOC440558(440558); CHST3(9469); LOC439985 |
| | | (439985); C10orf132(401647); MTG1(92170); LARP5(23185); C10orf38 |
| | | (221061); FGF8(2253); GALNAC4S-6ST(51363); DRD1IP(50632); |
| | | GNG3(2785); SSSCA1(10534); LRP5(4041); FADD(8772); CUL5(8065); H2AFX |
| | | (3014); LOC283174(283174); FKBP4(2288); DDX11(1663); FZD10 |
| | | (11211); DIP13B(55198); FBXO21(23014); CDC16(8881); C14orf132(56967); |
| | | TMEM121(80757); MDP-1(145553); EGLN3(112399); SOS2(6655); SAV1 |
| | | (60485); AKT1(207); FLJ25222(374666); NDN(4692); RAB40C(57799); |
| | | DNASE1(1773); ZNF646(9726); ELMO3(79767); WWOX(51741); FLJ45121 |
| | | (400556); AXIN1(8312); C16or128(65259); ARL6IP(23204); SIAH1(6477); |
| | | MGC4655(84752); TADA2L(6871); STAT5A(6776); FZD2(2535); YPEL2 |
| | | (388403); SEC14L1(6397); LRRC45(201255); ABR(29); TRPV3(162514); |
| | | CAMKK1(84254); PLSCR3(57048); TMEM107(84314); MGC23280(147015); |
| | | RETN(56729); APLP1(333); LIN7B(64130); LOC390980(390980); SIRT2 |
| | | (22938); ERCC2(2068); SHANK1(50944); LOC352909(352909); DHX |

Table 4-81

| | | |
|---|---|---|
| | | 35(60625); ARFGEF2(10564); MYT1(4661); TAF4(6874); DIDO1(11083); |
| | | SAMD10(140700); PKNTOX1(5316); PDXK(8566); KRTAP10-11(386678); |
| | | C21orf93(246704); TSSK2(23617); SERPIND1(3053); SMARCB1(6598); |
| | | PANX2(56666); LOC51233(51233); MN1(4330); APXL(357); STAG2(10735); |
| | | IL9R(3581); |
| hsa-miR-524* | 126; 928; | NBL1(4681); CDC42(998); KHDRBS1(10657); HDAC1(3065); RBBP4(5928); |
| | | GADD45A(1647); SH3GLB1(51100); CDC7(8317); VANGL1(81839); |
| | | BCL9(607); UHMK1(127933); FASLG(356); TGFB2(7042); TNFRSF9(3604); |
| | | CTNNBIP1(56998); E2F2(1870); FUSIF1(10772); CLSPN(63967); BCL10 |
| | | (8915); MCL1(4170); DEDD(9191); ASPM(259266); PPP1CB(5500); BIRC6 |
| | | (57448); PRKCE(5581); MSH2(4436); CCT7(10574); RIF1(55183); PMS1 |
| | | (5378); CREB1(1385); COL4A3(1285); YWHAQ(10971); E2F6(1876); |
| | | PUM2(23369); CARD12(58434); TIA1(7072); SEPT10(151011); IL1A(3552); |
| | | IL1B(3553); MCM6(4175); FRZB(2487); CLK1(1195); BARD1(580); FANCD2 |
| | | (2177); PCAF(8850); RBM5(10181); APPL(26060); TP73L(8626); OPA1 |
| | | (4976); RAF1(5894); NEK4(6787); UBE1C(9039); ATR(545); HD(3064); |
| | | CD38(952); HCAP-G(64151); SLC34A2(10568); EREG(2069); CCNG2(901); |
| | | FGF5(2250); GAK(2580); APBB2(323); CCNI(10983); DKK2(27123); |
| | | LEF1(51176); SFRP2(6423); CASP3(836); ERBB2IP(55914); CCNB1(891); |
| | | APC(324); CSNK1G3(1456); RAD50(10111); TCF7(6932); PRLR(5618); |
| | | BIRC1(4671); MCC(4163); ATG12(9140); PPP2CA(5515); NME5(8382); CDC25C |
| | | (995); CSNK1A1(1452); FBXW11(23291); RNF130(55819); E2F3(1871); |
| | | GMNN(51053); TTK(7272); TNFAIP3(7128); NEDD9(4739); DHX16 |
| | | (8449); DAXX(1616); CUL7(9820); DST(667); SESN1(27244); PLAGL1(5325); |
| | | LATS1(9113); AHR(196); CREB5(9586); EGFR(1956); LOC441257(441257); |
| | | HBP1(26959); BCAP29(55973); CALU(813); TRIAD3(54476); SOSTDC1 |
| | | (25928); SFRP4(6424); IGFBP3(3486); CDK6(1021); MCM7(4176); |
| | | PTN(5764); BNIP3L(665); ESCO2(157570); FZD3(7976); UNC5D(137970); |
| | | TACC1(6867); SULF1(23213); FZD6(8323); WHSC1L1(54904); DKK4 |
| | | (27121); TP53INP1(94241); MTSS1(9788); ANKRD15(23189); MGC21881 |
| | | (389741); C9orf47(286223); IFNA2(3440); GAS1(2619); SEMA4D(10507); |
| | | PPP6C(5537); C10orf9(219771); SIRT1(23411); PTEN(5728); FAS(355); KIF11 |
| | | (3832); FRAT1(10023); CSPG6(9126); TCF7L2(6934); DOCK1(1793); |
| | | PARD3(56238); DLG5(9231); DCLRE1A(9937); MKI67(4288); STIM1(6786); |
| | | WEE1(7465); HIPK3(10114); API5(8539); LRP5(4041); ATM(472); SLC1A2 |
| | | (6506); FZD4(8322); CTSC(1075); SESN3(143686); PPP2R1B(5519); |
| | | IAPP(3375); TEGT(7009); ING4(51147); BCAT1(586); FAIM2(23017); WIF1 |
| | | (11197); DUSP6(1848); DIP13B(55198); DIABLO(56616); CDK2AP1(8099); |
| | | HSMPP8(54737); TNFRSF19(55504); CDK8(1024); BRCA2(675); RFP2 |
| | | (10206); FLJ40296(122183); LATS2(26524); FOXO1A(2308); LIG4(3981); |
| | | BCL2L72(599); TXNDC(81542); CGRRF1(10668); PSEN1(5663); ACIN1 |
| | | (22985); GZMB(3002); EXOC5(10640); NEK9(91754); TGFB3(7043); CHES1 |
| | | (1112); FGF7(2252); MAPK6(5597); CSK(1445); IGF1R(3480); SLC12A6 |
| | | (9990); BCL2A1(597); DNAJA3(9093); CARD15(64127); CYLD(1540); |
| | | RBL2(5934); GSPT1(2935); FTS(64400); SLC12A4(6560); TXNL4B(54957); |
| | | PAFAH1B1(5048); NLK(51701); LIG3(3980); CDC6(990); FZD2(2535); |
| | | MAPT(4137); PRKCA(5578); MAP2K6(5608); BIRC5(332); NALP1(22861); |
| | | SMURF2(64750); CSNK1D(1453); PHLPP(23239); SMAD2(4087); CDH19 |
| | | (28513); MAP3K10(4294); AXL(558); BCL3(602); FLJ00060(90011); TNFSF14 |
| | | (8740); TRIB3(57761); CSNK2A1(1457); RBL1(5933); UBE2V1(7335); |
| | | AURKA(6790); CTCFL(140690); SON(6651); BRWD1(54014); MTP18 |
| | | (51537); MAPK1(5594); CHEK2(11200); MAGEH1(28986); ZC3H12B(340554); |
| | | MLLT7(4303); STAG2(10735); SEPT6(23157); CETN2(1069); |
| hsa-miR-525 | 127; 929; | TP73(7161); TNFRSF1B(7133); NBL1(4681); CDC42(998); RBBP4(5928); |
| | | MACF1(23499); CDC20(991); CDC14A(8556); CD2(914); BNIPL(149428); |
| | | DAP3(7818); PEA15(8682); PBX1(5087); FASLG(356); PPP1R12B(4660); |
| | | ADORA1(134); IL24(11009); CTNNBIP1(56998); FRAP1(2475); CASF9 |
| | | (842); RCC2(55920); E2F2(1870); YARS(8565); CLSPN(63967); NOTCH2 |
| | | (4853); DEDD(9191); C1orf32(387597); SELP(6403); ASPM(259266); TNNI1 |
| | | (7135); SLC41A1(254428); TP53BP2(7159); ZNF496(84838); BIRC6(57448); |
| | | PRKCE(5581); MSH2(4436); HTRA2(27429); HK2(3099); TCF7L1(83439); |
| | | BRRN1(23397); RIF1(55183); TAIP-2(80034); ZAK(51776); CD28(940); |
| | | ALS2CR19(117583); ANKZF1(55139); COL4A3(1285); PSMD1(5707); |
| | | E2F6(1876); CARD12(58484); TIA1(7072); EDAR(10913); ANAPC1(64682); |
| | | IL1B(3553); STAT1(6772); PAX3(5077); CTNNB1(1499); GNL3(2635 |

Table 4-82

4); IL17RB(55540); APPL(26060); MCM2(4171); NEK11(79858); ACPP(55);
FAIM(55179); FXR1(8087); TP73L(8626); OPA1(4976); CIDEC(63924);
RAF1(5894); MRPS25(64432); AXUD1(64651); SEC22L3(9117); NME6(10201);
PROK2(60675); VGLL3(389136); STAG1(10274); PIK3CB(5291); TFDP2
(7029); ATR(545); HD(3064); CD38(952); ANAPC4(29945); EREG(2069);
CCNG2(901); FGF5(2250); GAK(2580); PPBP(5473); PRKG2(5593);
UNC5C(8633); DKK2(27123); NEK1(4750); CASP3(836); SKP2(6504); NIPBL
(25836); CARD6(84674); PIK3R1(5295); APC(324); CSNK1G3(1456);
RAD50(10111); WDR55(54853); UNC5A(90249); PRLR(5618); UNG2(10309);
BIRC1(4671); MCC(4163); PPP2CA(5515); CD14(929); HTR4(3360); FBXW11
(23291); LY86(9450); E2F3(1871); GMNN(51053); LTA(4049); CDKN1A
(1026); PARC(23113); PTP4A1(7803); FOXO3A(2309); SASH1(23328);
NEDD9(4739); ATXN1(6310); DHX16(8449); MDC1(9656); IER3(8870);
IHPK3(117283); CCND3(896); PKHD1(5314); MCM3(4172); DST(667);
ATG5(9474); FRK(2444); SGK(6446); PLAGL1(5325); LATS1(9113); TFB1M
(51106); SEPT7(989); EGFR(1956); FZD9(8326); STAG3(10734); CASP2
(835); MAD1L1(8379); TRIAD3(54476); PMS2(5395); ELMO1(9844); YWHAG
(7532); CDK6(1021); MCM7(4176); HIPK2(28996); POLR3D(661); PTK2B
(2185); UNC5D(137970); TACC1(6867); WISP1(8840); LZTS1(11178);
TNFRSF10B(8795); TNFRSF10A(8797); ENTPD4(9583); CLU(1191);
STK3(6788); MTSS1(9788); ANKRD15(23189); JAK2(3717); RECK(8434);
DAPK1(1612); GADD45G(10912); ABL1(25); CDKN2A(1029); CDKN2B
(1030); TAF1L(138474); BAG1(573); SEMA4D(10507); PTCH(5727); CTNNAL1
(8727); TRAF1(7185); TSC1(7248); ANAPC2(29882); ZMYND11(10771);
C10orf9(219771); SGPL1(8879); UNC5B(219699); PTEN(5728); FRAT1
(10023); LZTS2(84445); SUFU(51684); PDCD11(22984); CSPG6(9126);
BAG3(9531); DOCK1(1793); SFMBT2(57713); PARD3(56288); C10orf46
(143384); MKI67(4288); BNIP3(664); STIM1(6786); WEE1(7465); F2(2147);
BIRC2(329); ATM(472); CD3E(916); CBL(867); LOH11CR2A(4013); HRAS
(3265); ZNF289(84364); CDCA5(113130); MAP3K11(4296); RELA(5970);
MTL5(9633); PAK1(5058); FZD4(8322); SESN3(143686); ICEBERG(59082);
PPP2R1B(5519); BCL9L(283149); SCN3B(55800); LTBR(4055); TNFRSF7
(939); CNAP1(9918); BCL2L14(79370); TEGT(7009); AQP2(359);
ESPL1(9700); PDE1B(5153); PA2G4(5036); MDM2(4193); SOCS2(8835);
APAF1(317); KNTC1(9735); TNFRSF1A(7132); LRP6(4040); HDAC7A(51564);
MCRS1(10445); KIAA0286(23306); WIF1(11197); PAWR(5074); PPP1CC
(5501); ANAPC5(51433); MPHOSPH9(10198); CHFR(55743); HSMPP8
(54737); BRCA2(675); RB1(5925); FLJ30707(220108); ING1(3621); TFDP1
(7027); GALNTL1(57452); PSEN1(5663); VASH1(22846); TCL6(27004);
KIAA0125(9834); ACIN1(22985); CIDEB(27141); GZMB(3002); EGLN3
(112399); LTBP2(4053); PGF(5228); CHES1(1112); PPP1R13B(23368);
AKT1(207); BUB1B(701); NDN(4692); BMF(90427); BCL2L10(10017);
CA12(771); TBL3(10607); TSC2(7249); CCNF(899); DNAJA3(9093); PRRT2
(112476); PAPD5(64282); CYLD(1540); NOL3(8996); ASCIZ(23300); ZNF276
(92822); GRIN2A(2903); MAPK3(5595); PYCARD(29108); SIAH1
(6477); CIAPIN1(57019); CSNK2A2(1459); APPBP1(8883); RBM35B(80004);
TXNL4B(54957); PCOLN3(5119); PAFAH1B1(5048); GSG2(83903); ALOX12
(239); ALOX15B(247); TRAF4(9618); NF1(4763); MLX(6945); FZD2
(2535); MAPT(4137); DLX4(1748); CROP(51747); PCTP(58488); DYNLL2
(140735); PRKCA(5578); SPHK1(8877); BIRC5(332); CARD14(79092); C17orf27
(57674); MNT(4335); P2RX1(5023); NALP1(22861); GAS7(8522);
SPAG5(10615); HDAC5(10014); TBC1D16(125058); CIDEA(1149); MAPRE2
(10982); DCC(1630); PHLPP(23239); ENOSF1(55556); TXNL1(9352);
BCL2(596); PARD6G(84552); FZR1(51343); HMG20B(10362); GIPC3(126326);
TNFSF9(8744); DNM2(1785); CASP14(23581); UBA52(7311); CSPG3
(1463); APLP1(333); MAP3K10(4294); BAX(581); ATF5(22809); PPP2R1A
(5518); NALP2(55655); TNFSF14(8740); MGC71805(403113); ZNF443
(10224); PRDX2(7001); DNASE2(1777); SIRT2(22933); LYPD5(284348);
ERCC2(2068); KLK10(5655); MCM8(84515); PLCB1(23236); CEP250(11190);
UBE2C(11065); BCL2L1(598); RIMS4(140730); AURKA(6790); DIDO1
(11083); SON(6651); SNF1LK(150094); POFUT2(23275); BCL2L13
(23786); CDC45L(8318); SMARCB1(6598); KREMEN1(83999); NF2(4771);
YWHAH(7533); PPARA(5465); BID(637); DGCR2(9993); MAPK1(5594);
PPM1F(9647); MN1(4330); CHEK2(11200); CARD10(29775); DMC1(11144);
PDGFB(5155); C22orf9(23313); SMC1L2(27127); FLJ41993(400935)

Table 4-83

| | | |
|---|---|---|
| | | ; HDAC6(10013); CCNB3(85417); MLLT7(4303); BIRC4(331); IKBKG(8517); |
| | | SMC1L1(8243); BTK(695); SEPT6(23157); ARHGEF6(9459); HCFC1 |
| | | (3054); |
| hsa-miR-526a | 128; 930; 931; | SESN2(83667); RBBP4(5928); MACF1(23499); SH3GLB1(51100); RAP1A |
| | 932; 933; | (5906); IPO9(55705); CCNL2(81669); CASP9(842); MUTYH(4595); NRAS |
| | 934; 935; | (4893); NMNAT2(23057); TP53BP2(7159); BRRN1(23397); RIF1(55183); |
| | 936; 937; 938; | ZAK(51776); CFLAR(8837); CASP10(843); EDAR(10913); BUB1(699); CUL3 |
| | | (8452); FANCD2(2177); CTNNB1(1499); APPL(26060); ACPP(55); TADA3L |
| | | (10474); PIK3CB(5291); NFKB1(4790); TLR2(7097); SNCA(6622); |
| | | CASP3(836); ERBB2IP(55914); RAD17(5884); APC(324); SEMA6A(57556); |
| | | FBXW11(23291); GMNN(51053); CSNK2B(1460); CDC5L(988); TTK(7272); |
| | | TNFAIP3(7128); MDC1(9656); DST(667); SESN1(27244); BCLAF1 |
| | | (9774); HDAC9(9734); FLJ10099(55069); BCAP29(55973); PMS2(5395); CARD4 |
| | | (10392); ELMO1(9844); SFRP4(6424); ESCO2(157570); RIPK2(8767); |
| | | WISP1(8840); TNFRSF10B(8795); ADRA1A(148); ANKRD15(23189); |
| | | NTRK2(4915); TNFSF15(9966); DKK1(22943); CDC2(983); BTRC(8945); |
| | | CSPG6(9126); MKI67(4288); STIM1(6786); HIPK3(10114); LGALS12(85329); |
| | | DPF2(5977); SSSCA1(10534); BIRC2(329); ATM(472); PTH(5741); MTL5 |
| | | (9633); SHANK2(22941); CACNA1C(775); ESPL1(9700); CCT2(10576); |
| | | APAF1(317); GAS2L3(283431); BCAT1(586); LALBA(3906); ANAPC7 |
| | | (51434); ANAPC5(51433); MPHOSPH9(10198); TNFRSF19(55504); ATP8A2 |
| | | (51761); BRCA2(675); SUGT1(10910); CDC16(8881); FOXO1A(2308); |
| | | BCL2L2(599); TXNDC(81542); EGLN3(112399); BAG5(9529); FGF7(2252); |
| | | TSC2(7249); TRAF7(84231); CARD15(64127); CYLD(1540); HIC1(3090); |
| | | TNFSF12(8742); MAPK7(5598); TRAF4(9618); MAPT(4137); NGFR(4804); |
| | | ANAPC11(51529); P2RX1(5023); SPAG5(10615); PIP5K2B(8396); HDAC5 |
| | | (10014); MPO(4353); CBX4(8535); MALT1(10892); PARD6G(84552); |
| | | GIPC3(126326); AXL(558); BCL2L12(83596); NALP2(55655); MCM8(84515); |
| | | CEP250(11190); SCAND1(51282); CTCFL(140690); CABLES2(81928); |
| | | DIDO1(11083); SON(6651); ADARB1(104); BCL2L13(23786); EP300 |
| | | (2033); MN1(4330); SMC1L2(27127); CD40LG(959); SMC1L1(8243); PDCD8 |
| | | (9131); |
| hsa-miR-527 | 129; 939; | CDC42(998); MACF1(23499); NFYC(4802); CDC20(991); GADD45A(1647); |
| | | RAP1A(5906); SYCP1(6847); VANGL1(81839); BCL9(607); BNIPL(149428); |
| | | SYT11(23208); PEA15(8682); UHMK1(127933); FAM20B(9917); CEP350 |
| | | (9857); ADORA1(134); CCNL2(81669); CTNNBIP1(56998); FRAP1 |
| | | (2475); CASP9(842); ROC2(55920); E2F2(1870); YARS(8565); SLC35D1(23169); |
| | | PAP2D(163404); SASS6(163786); AMIGO1(57463); MCL1(4170); GLT25D2 |
| | | (23127); FH(2271); LPIN1(23175); RHOB(388); PPP1CB(5500); BIRC6 |
| | | (57448); HTRA2(27429); RIF1(55183); ZAK(51776); CFLAR(8837); FZD7 |
| | | (8324); PTMA(5757); CARD12(58484); RTN4(57142); TGFA(7039); MCM6 |
| | | (4175); ACVR1(90); IFIH1(64135); CERKL(375298); NCKAP1(10787); |
| | | USP37(57695); HDAC4(9759); FANCD2(2177); HRH1(3269); GNL3(26354); |
| | | APPL(26060); RAP2B(5912); TP73L(8626); RAF1(5894); NEK4(6787); |
| | | PIK3CB(5291); ATR(545); WHSC1(7468); HD(3064); HCAP-G(64151); |
| | | SEPT11(55752); SPP1(6696); APBB2(323); CCN1(10983); PRKG2(5593); |
| | | UNC5C(8633); DKK2(27123); SLC7A11(23657); SETD7(80854); OTUD4 |
| | | (54726); SFRP2(6423); NEK1(4750); CARD6(84674); CCNB1(891); TNPO1 |
| | | (3842); RAD50(10111); G3BP(10146); PRLR(5618); MCC(4163); ATG12 |
| | | (9140); IRF1(3659); FGF1(2246); CSNK1A1(1452); FBXW11(23291); DDX41 |
| | | (51428); RNF130(55819); CSNK2B(1460); PTP4A1(7803); FOXO3A(2309); |
| | | TNFAIP3(7128); SASH1(23328); ATXN1(6310); BAK1(578); SUPT3H |
| | | (8464); TNFRSF21(27242); MCM3(4172); DST(667); ATG5(9474); FRK(2444); |
| | | BCLAF1(9774); LATS1(9113); AGPAT4(56895); PDE10A(10846); LFNG |
| | | (3955); FOXK1(221937); HBP1(26959); CUL1(8454); CDK6(1021); CDK5 |
| | | (1020); FASTK(10922); POLR3D(661); SLC25A37(51312); PTK2B(2185); |
| | | UNC5D(137970); TACC1(6867); UBE2V2(7336); RIPK2(8767); MYC(4609); |
| | | WISP1(8840); PSD3(23362); BIN3(55909); TNFRSF10B(8795); TNFRSF10D |
| | | (8793); DKK4(27121); TP53INP1(94241); EXT1(2131); ANKRD15 |
| | | (23189); UNC13B(10497); INVS(27130); IFNA2(3440); CDKN2B(1030); |
| | | KIAA0367(23273); BICD2(23299); CTNNAL1(8727); FBXW2(26190); TRAF1 |
| | | (7185); CARD9(64170); ZMYND11(10771); SIRT1(23411); PTEN(5728); |
| | | FAS(355); KIF11(3832); FRAT1(10023); SUFU(51684); PDCD11(22984); |
| | | TCF7L2(6934); ZWINT(11130); C10orf46(143384); MKI67(4288); IFTTM1 |
| | | (8519); TEAD1(7003); API5(8539); EXT2(2132); KIAA1377(57562); AT |

Table 4-84

| | | |
|---|---|---|
| | | M(472); DKK3(27122); HRASLS3(11145); MTL5(9633); FZD4(8322); SESN3 |
| | | (143686); BTG4(54766); PPP2R1B(5519); BCL9L(283149); C11orf45(219833); |
| | | CNAP1(9918); RBMS2(5939); MDM2(4193); SOCS2(8835); APAF1 |
| | | (317); CHST11(50515); KNTC1(9735); FZD10(11211); LRP6(4040); BCAT1 |
| | | (586); HDAC7A(51564); FAIM2(23017); CDK4(1019); WIF1(11197); BTG1 |
| | | (694); PPTC7(160760); MPHOSPH9(10198); BRCA2(675); AKAP11(11215); |
| | | RFP2(10206); ING1(3621); CDC16(8881); FLJ25477(219287); NEK3 |
| | | (4752); KLF12(11278); LIG4(3981); PNN(5411); CGRRF1(10668); C14orf35 |
| | | (122830); VASH1(22846); NOVA1(4857); CDKL1(8814); DLG7(9787); EXOC5 |
| | | (10640); LTBP2(4053); CHES1(1112); CCNB2(9133); IGF1R(3480); |
| | | NDN(4692); BMF(90427); PRTG(283659); DAPK2(23604); DNASE1(1773); |
| | | DNAJA3(9093); PAPD5(64282); RBL2(5934); CTCF(10664); FAM100A |
| | | (124402); DNAJA2(10294); SLC12A4(6560); TERF2(7014); ALOX12(239); |
| | | MAPK7(5598); NLK(51701); NF1(4763); CCL2(6347); MAPT(4137); PRKCA |
| | | (5578); CDK3(1018); BIRC5(332); CARD14(79092); ANAPC11(51529); |
| | | MNT(4335); NALP1(22861); GPS2(2874); GAS7(8522); AP1GBP1(11276); |
| | | HDAC5(10014); SEPT4(5414); PTRH2(51651); ERN1(2081); HELZ(9931); |
| | | SOCS3(9021); CSNK1D(1453); C18orf1(753); MIB1(57534); CABLES1 |
| | | (91768); MAPK4(5596); DCC(1630); BCL2(596); TXNL4A(10907); PARD6G |
| | | (84552); GADD45B(4616); APOE(348); CDKN2D(1032); CARD8(22900); |
| | | MCM8(84515); PLCB1(23236); MAPRE1(22919); CEP250(11190); STK4 |
| | | (6789); PARD6B(84612); E2F1(1869); AURKA(6790); DIDO1(11083); USP16 |
| | | (10600); SON(6651); MX1(4599); APP(351); ITGB2(3689); CDC45L(8318); |
| | | NF2(4771); TNRC6B(23112); MN1(4330); RBM9(23543); TOB2(10766); |
| | | HDAC6(10013); CCNB3(85417); MLLT7(4303); NGFRAP1(27018); BIRC4 |
| | | (331); STAG2(10735); RP6-213H19.1(51765); AFF2(2334); DKC1(1736); |
| | | SEPT6(23157); CUL4B(8450); DAZ2(57055); |
| hsa-miR-411 | 2057; 2115; | SAMD11(148398); TNFRSF1B(7133); HDAC1(3065); MACF1(23499); FLJ45337 |
| | 2116; | (400754); SH3GLB1(51100); RAP1A(5906); ADORA1(134); IL19(29949); |
| | | TGFB2(7042); TNFRSF9(3604); FRAP1(2475); RCC2(55920); DBT |
| | | (1629); ASPM(259266); TP53BP2(7159); LOC165186(165186); BIRC6(57448); |
| | | RHOQ(23433); SLC9A2(6549); RIF1(55183); CASP10(843); GFPT1 |
| | | (2673); NCKAP1(10787); FZD5(7855); CUL3(8452); FANCD2(2177); PCAF |
| | | (8850); IL17RB(55540); APPL(26060); RAF1(5894); SLC4A7(9497); TRAIP |
| | | (10293); FHIT(2272); MGC40579(256356); ATR(545); GP5(2814); HCAP- |
| | | G(64151); FLJ14001(79730); SEPT11(55752); CDS1(1040); PRKG2(5593); |
| | | UNC5C(8633); LEF1(51176); TNPO1(3842); APC(324); TCF7(6932); PHF15 |
| | | (23338); UNC5A(90249); FEM1C(56929); ATG12(9140); SEMA6A(57556); |
| | | FBXW11(23291); DUSP1(1843); E2F3(1871); ABT1(29777); LTA(4049); |
| | | TNF(7124); PARC(23113); TUBB2B(347733); IER3(8870); SUPT3H |
| | | (8464); DST(667); ASCC3(10973); ARHGAP18(93663); MAP3K5(4217); PDCD2 |
| | | (5134); STK17A(9263); EGFR(1956); FZD9(8326); FZD1(8321); CASP2 |
| | | (835); CDK6(1021); MGC14289(92092); UBE2V2(7336); TNFRSF10A |
| | | (8797); DAPK1(1612); RAD23B(5887); GPR107(57720); SEMA4D(10507); |
| | | TSC1(7248); ANAPC2(29882); UNC5B(219699); KIF11(3832); BTRC(8945); |
| | | BAG3(9531); PARD3(56288); FZD8(8325); BNIP3(664); LRP5(4041); |
| | | BIRC3(330); CD3G(917); EIF4G2(1982); CD59(966); CDCA5(113130); RAB30 |
| | | (27314); FZD4(8322); PPP2R1B(5519); BCL2L14(79370); FZD10(11211); |
| | | MMP19(4327); PAWR(5074); PCTK2(5128); FLJ20674(54621); SCARB1 |
| | | (949); FGF9(2254); CDC16(8881); SLC7A1(6541); FOXOL1A(2308); VASH1 |
| | | (22846); NRXN3(9369); EGLN3(112399); SDCCAG1(9147); SYNJ2BP |
| | | (55333); CSNK1G1(53944); FLJ22795(80154); TSC2(7249); TRAF7(84231); |
| | | CYLD(1540); BOLA2(552900); LOC283849(283849); SLC12A4(6560); |
| | | TXNL4B(54957); TRAF4(9618); NF1(4763); TADA2L(6871); CROP(51747); |
| | | NME1(4830); SPHK1(8877); GPS1(2873); GAS7(8522); BRCA1(672); |
| | | SEPT4(5414); ARHGAP28(79822); CABLES1(91768); SERPINB2(5055); |
| | | CSNK1G2(1455); TNFSF9(8744); DNM2(1785); EGFL4(1954); EMR3(84658); |
| | | ERCC2(2068); FUT1(2523); CGB7(94027); SNPH(9751); UBE2C(11065); |
| | | PARD6B(84612); RBL1(5933); CHAF1B(8208); MX1(4599); POFUT2 |
| | | (23275); KREMEN1(83999); NF2(4771); SEPT3(55964); MAPK1(5594); |
| | | HDAC6(10013); SMC1L1(8243); WNK3(65267); CHM(1121); CUL4B(8450); |
| | | ARHGEF6(9459); |
| hsa-miR-421 | 2058; 2117; | DFFB(1677); VAMP3(9341); PQLC2(54896); DHDDS(79947); SFN(2810); |
| | | KHDRBS1(10657); LCK(3932); EIF2C4(192670); MACF1(23499); ERMAP |
| | | (114625); DIO1(1733); PTGFR(5737); FNBP1L(54874); SLC30A7(14886 |

Table 4-85

7); PRUNE(58497); DAP3(7818); UHMK1(127933); RALGPS2(55103); SMYD2
(56950); EDARADD(128178); KMO(8564); TNFRSF14(8764); CTNNBIP1
(56998); DFFA(1676); FRAP1(2475); E2F2(1870); FUSIP1(10772);
YARS(8565); TESK2(10420); DAB1(1600); SERBP1(26135); FUBP1(8880);
GFI1(2672); NRAS(4893); NOTCH2(4853); S100A2(6273); ILF2(3608);
KCNN3(3782); MEF2D(4209); CD5L(922); SH2D1B(117157); IL10(3586);
LPGAT1(9926); TP53BP2(7159); EGLN1(54583); YPEL5(51646); BIRC6
(57448); MSH2(4436); CCT7(10574); FLJ13910(64795); POLR1B(84172);
RAB3GAP1(22930); RIF1(55183); PMS1(5378); GLS(2744); DNAPTP6
(26010); CFLAR(8837); CASP10(843); PSMD1(5707); SEPT2(4735); RNASEH1
(246243); E2F6(1876); WDR35(57539); CARD12(58484); STRN(6801);
EIF2AK2(5610); PREPL(9581); MGAT4A(11320); AFF3(3899); TGFBRAP1
(9392); BUB1(699); FLJ10996(54520); WDR33(55339); POLR2D(5433);
CACNB4(785); IFIH1(64135); NCKAP1(10787); STAT1(6772); BARD1
(580); CUL3(8452); HDAC4(9759); NDUFA10(4705); VHL(7428); NR2C2
(7182); NR1D2(9975); FBXL2(25827); PDCD6IP(10015); RPL14(9045); LIMD1
(8994); CCR9(10803); GNL3(26354); APPL(26060); IGSF4D(253559);
PCNP(57092); UPK1B(7348); MRAS(22808); SMC4L1(10051); TP73L(8626);
IL1RAP(3556); OPA1(4976); THRB(7068); AXUD1(64651); C3orf62
(375341); MAGI1(9223); UBE1C(9039); TOMM70A(9868); POLQ(10721); ITGB5
(3693); STAG1(10274); XRN1(54464); ATR(545); SERP1(27230); LOC285382
(285382); PCYT1A(5130); WHSC1(7468); HCAP-G(64151); SEPT11
(55752); CCNG2(901); WWC2(80014); APBB2(323); CCNI(10983); DNAJB14
(79982); ANKRD50(57182); SFRP2(6423); NEK1(4750); MFAP3L
(9848); CASP3(836); FAM105B(90268); NIPBL(25836); CARD6(84674); ERBB2IP
(55914); APC(324); LOC133619(133619); ADAMTS19(171019); RAD50
(10111); PCDHB5(26167); PCDHB13(56123); PCDHGB4(8641); MAML1
(9794); PRLR(5618); PRKAA1(5562); LYSMD3(116068); ELL2(22936);
ST8SIA4(7903); SEMA6A(57556); AFF4(27125); CDC23(8697); CDC25C
(995); NR3C1(2908); CSNK1A1(1452); PDE6A(5145); CD74(972); FBXW11
(23291); FLJ16171(441116); RNF130(55819); RIPK1(8737); LY86(9450);
FAM8A1(51439); GMNN(51053); NFYA(4800); BAG2(9532); COL19A1
(1310); ANKRD6(22881); FOXO3A(2309); HECA(51696); SASH1(23328);
PLEKHG1(57480); QKI(9444); CCND3(896); DST(667); BACH2(60468);
CCNC(892); FRK(2444); ECHDC1(55862); SGK(6446); DEADC1(134637);
KATNA1(11104); LATS1(9113); IQCE(23288); FOXK1(221937); STK17A
(9263); ADCY1(107); EGFR(1956); ZNF655(79027); ZNF498(221785); RINT1
(60561); PMS2(5395); SOSTDC1(25928); OSBPL3(26031); YWHAG
(7532); CDK6(1021); ZC3HC1(51530); HIPK2(28996); SLC37A3(84255);
TPK1(27010); CLN8(2055); NEIL2(252969); PIWIL2(55124); PDLIM2(64236);
BNIP3L(665); UNC5D(137970); BAG4(9530); TACC1(6867); WISP1
(8840); ADRA1A(148); CLU(1191); DUSP4(1846); WHSC1L1(54904); RUNX1T1
(862); STK3(6788); JRK(8629); ANKRD15(23189); DMRT2(10655);
UBE2R2(54926); GADD45G(10912); INVS(27130); CDK9(1025); RPL7A
(8130); SMU1(55234); TRPM3(80036); KIAA0367(23273); SEMA4D(10507);
RAPGEF1(2889); ANAPC2(29882); ZMYND11(10771); ARL5B(221079);
EIF4EBP2(1979); SGPL1(8879); KIF11(3832); ENTPD1(953); SCD
(6319); BTRC(8945); NOLC1(9221); SUFU(51684); LOC387646(387646); PARD3
(56288); ZNF11B(7558); RHOBTB1(9886); ARHGAP19(84986); FBXW4
(6468); SMNDC1(10285); DCLRE1A(9937); C10orf46(143384); MKI67
(4288); TRIM6(117854); WEE1(7465); HIPK3(10114); INCENP(3619);
SCGB2A1(4246); DPF2(5977); RBM14(10432); BIRC3(330); ATM(472); UBE4A
(9354); MLL(4297); CBL(867); STS-1(84959); CHEK1(1111); SIRT3
(23410); PDDC1(347862); SLC25A22(79751); TOLLIP(54472); EIF4G2(1982);
CDCA5(113130); CLCF1(23529); SUV420H1(51111); RAB30(27314);
FZD4(8322); CTSC(1075); SESN3(143686); CNAP1(9918); BCL2L14(79370);
LEMD3(23592); CCT2(10576); SOCS2(8835); SLC25A3(5250); APAF1
(317); SLC17A8(246213); KNTC1(9735); EP400NL(347918); JARID1A
(5927); PARP11(57097); NOL1(4839); HDAC7A(51564); DDIT3(1649); GNS
(2799); BTG1(694); DIP13B(55198); PPP1CC(5501); HRK(8739); FLJ20674
(54621); TMEM132D(121256); ATP8A2(51761); BRCA2(675); RB1(5925);
RAP2A(5911); ARHGEF7(8874); CSNK1A1L(122011); EPSTI1(94240);
RCBTB1(55213); ATP7B(540); KLF12(11278); TBC1D4(9882); ABCC4
(10257); BCL2L2(599); DACT1(51339); MNAT1(4331); EIF2S1(1965); PSEN1
(5663); C14orf111(51077); TSHR(7253); STRN3(29966); CFL2(1073);

Table 4-86

| | | |
|---|---|---|
| | | NFKBIA(4792); NEK9(91754); TGFB3(7043); CHES1(1112); C14orf103(55102); |
| | | BCL11B(64919); BAG5(9529); JAG2(3714); FGF7(2252); MAPK6(5597); |
| | | ZNF609(23060); HMG20A(10363); SCAND2(54581); IGF1R(3480); |
| | | BMF(90427); ZFP106(64397); BCL2A1(597); NMB(4828); LYSMD4(145748); |
| | | TSC2(7249); DNASE1(1773); DNAJA3(9093); SNN(8303); CARD15(64127); |
| | | CYLD(1540); RBL2(5934); ELMO3(79767); NFAT5(10725); ZNF200 |
| | | (7752); GSPT1(2935); LOC440350(440350); SIAH1(6477); CSNK2A2(1459); |
| | | APPBP1(8883); TXNL4B(54957); SRR(63826); PAFAH1B1(5048); ALOX12 |
| | | (239); HS3ST3B1(9953); TRAF4(9618); GOSR1(9527); SOCS7(30837); |
| | | CDC6(990); MAPT(4137); PNPO(55163); KPNA2(3838); BIRC5(332); ANAPC11 |
| | | (51529); GPS1(2873); CRK(1398); TP53(7157); GAS7(8522); FLJ36492 |
| | | (284047); GAS2L2(246176); TBC1D3C(414060); KA36(125115); HDAC5 |
| | | (10014); PHB(5245); PECAM1(5175); GNA13(10672); RECQL5(9400); |
| | | UBE2O(63893); NPTX1(4884); MAFG(4097); CETN1(1068); VAPA(9218); |
| | | C18orf1(753); CABLES1(91768); MAPRE2(10982); SMAD4(4089); SERPINB8 |
| | | (5271); DOK6(220164); FAM59A(64762); SMAD2(4087); MBD2 |
| | | (8932); GADD45B(4616); FZR1(51343); UHRF1(29128); LOC390980(390980); |
| | | ZNF548(147694); PRX(57716); PLCB1(23236); POFUT1(23509); MAPRE1 |
| | | (22919); CEP250(11190); C20orf118(140711); CTNNBL1(56259); |
| | | CD40(958); CSE1L(1434); STX16(8675); AURKA(6790); TMEPAI(56937); |
| | | SYCP2(10388); DIDO1(11083); APP(351); BCL2L13(23786); ADRBK2(157); |
| | | LOC400924(400924); MTP18(51537); YWHAH(7533); TNRC6B(23112); |
| | | GTSE1(51512); BID(637); DERL3(91319); PDGFB(5155); NAGA(4668); |
| | | C22orf9(23313); SMC1L2(27127); BIRC4(331); HMGB3(3149); IKBKG(8517); |
| | | DKC1(1736); BRCC3(79184); PRKX(5613); FAM9C(171484); PIGA |
| | | (5277); SPIN2(54466); CUL4B(8450); BCAP31(10134); MECP2(4204); PRKY |
| | | (5616); UTY(7404); |
| hsa-miR-425-5p | 2059; 2118; | DFFB(1677); TNFRSF1B(7133); PAX7(5081); MAN1C1(57134); RBBP4 |
| | 2123; 2124; | (5928); MACF1(23499); PLK3(1263); RNF11(26994); CDC7(8317); CDC14A |
| | 2315; 2316; | (8556); SYCP1(6847); VANGL1(81839); S100A7L1(338324); MPZL1(9019); |
| | | C1orf21(81563); IL24(11009); EDARADD(128178); TNFRSF9(3604); |
| | | CTNNBIP1(56998); FRAP1(2475); MTHFR(4524); FOXJ3(22887); SLC35D1 |
| | | (23169); PTGER3(5733); GCLM(2730); SASS6(163786); NRAS(4893); |
| | | NOTCH2(4853); NBPF11(200030); TPM3(7170); KLF11(8462); PRKCE |
| | | (5581); VRK2(7444); ANTXR1(84168); RIF1(55183); ZAK(51776); CASP10 |
| | | (843); PSMD1(5707); SEPT2(4735); RNASEH1(246243); YWHAQ(10971); |
| | | E2F6(1876); TLK1(9874); ATP5G3(518); FLJ39502(285025); CUL3(8452); |
| | | HDAC4(9759); HDLBP(3069); ENDOGL1(9941); APPL(26060); DTX3L |
| | | (151636); SELT(51714); SMC4L1(10051); USP13(8975); THRB(7068); IHPK2 |
| | | (51447); LRIG1(26018); AER61(285203); PIK3CB(5291); ATR(545); SIAH2 |
| | | (6478); FLJ37478(339983); HD(3064); KLF3(51274); CXCL6(6372); |
| | | AFF1(4299); TLR2(7097); APBB2(323); UGT2B15(7366); CASP3(836); SKP2 |
| | | (6502); MARVELD2(153562); SCAMP1(9522); TCF7(6932); SMAD5 |
| | | (4090); PCDHB5(26167); CPLX2(10814); DNAJC18(202052); FBXW11(23291); |
| | | HECA(51696); QKI(9444); RP3-398D13.1(285780); DAXX(1616); SUPT3H |
| | | (8464); TRAM2(9697); DST(667); STX7(8417); MAP3K5(4217); PDE10A |
| | | (10846); HDAC9(9734); SEPT7(989); ADCY1(107); FZD1(8321); RBM33 |
| | | (155435); PMS2(5395); TMED4(222068); PURB(5814); IGFBP3(3486); |
| | | PMS2L2(5380); MGC40405(257415); CDK6(1021); CNOT4(4850); UNC5D |
| | | (137970); UBE2V2(7336); SULF1(23213); TERF1(7013); CHRAC1(54108); |
| | | DUSP4(1846); FUT10(84750); SFRP1(6422); JAK2(3717); C9orf47(286223); |
| | | FREQ(23413); TAF1L(138474); PTCH(5727); ABCA1(19); SIRT1(23411); |
| | | PTEN(5728); FAS(355); BTRC(8945); CSPG6(9126); DNMT2(1787); |
| | | PARD3(56288); C10orf72(196740); ANK3(288); C10orf13(143282); FBXW4 |
| | | (6468); SH3PXD2A(9644); IFTTM1(8519); MLSTD2(84188); HIPK3(10114); |
| | | API5(8539); EXT2(2132); VEGFB(7423); ATM(472); SNF1LK2(23235); |
| | | CBL(867); LOH11CR2A(4013); ARFIP2(23647); NRIP3(56675); DKK3 |
| | | (27122); HRASLS3(11145); FGF3(2248); PGM2L1(283209); HBXAP(51773); |
| | | CTSC(1075); BTG4(54766); C12orf32(83695); PTMS(5763); PTPN6(5777); |
| | | PEX5(5830); BCL2L14(79370); GLI1(2735); MDM2(4193); GCT2(10576); |
| | | ATP6V0A2(23545); DUSP16(80824); BCAT1(586); KRAS(3845); MCRS1 |
| | | (10445); C12orf22(81566); IGF1(3479); NT5DC3(51559); MPHOSPH9 |
| | | (10198); TMEM132D(121256); FGF9(2254); TNFRSF19(55504); BRCA2 |
| | | (675); RFP2(10206); CLYBL(171425); CDC16(8881); PSEN1(5663); VASH1 |
| | | (22846); EGLN3(112399); NEK9(91754); CHES1(1112); MOAP1(64112); |

Table 4-87

| | | |
|---|---|---|
| | | MAPK6(5597); SLC12A6(9990); BMF(90427); BNIP2(663); CSNK1G1(53944); |
| | | BCL2A1(597); FLJ22795(80154); CRAMP1L(57585); ABAT(18); LOC63928 |
| | | (63928); ATP2A1(487); SPN(6693); NFATC3(4775); BANP(54971); |
| | | PKMYT1(9088); LITAF(9516); LKAP(9665); SMG1(23049); USP31(57478); |
| | | SLC12A4(6560); PAFAH1B1(5048); GSG2(83903); TNFSF12(8742); NGFR |
| | | (4804); HLF(3131); PPM1D(8493); MAP2K6(5608); TIAF1(9220); NUFIP2 |
| | | (57532); TCF2(6928); CDC27(996); CYB561(1534); RAB31(11031); |
| | | DCC(1630); MALT1(10892); DOK6(220164); DYM(54808); BCL2(596); FZR1 |
| | | (51343); MCEMP1(199675); DNM2(1785); UPF1(5976); DPY19L3(147991); |
| | | APOE(348); DAPK3(1613); TGFB1(7040); CARD8(22900); LOC284296 |
| | | (284296); PLCB1(23236); MAPRE1(22919); CEP250(11190); CD40(958); |
| | | DNAJC5(80331); C20orf42(55612); ZHX3(23051); NCOA5(57727); BTG3 |
| | | (10950); CYYR1(116159); BRWD1(54014); LSS(4047); SEPT3(55964); MAPK1 |
| | | (5594); PPM1F(9647); MN1(4330); CBX6(23466); SMC1L2(27127); |
| | | WWC3(55841); AGTR2(186); FIGF(2277); SMC1L1(8243); OPHN1(4983); |
| | | BTK(695); LAMP2(3920); CUL4B(8450); PDCD8(9131); ARHGEF6(9459); |
| | | PRKY(5616); |
| hsa-miR-454-5p | 2060; | LIN28(79727); RAP1A(5906); RIMS3(9783); MCL1(4170); ASH1L(55870); |
| | | WDR42A(50717); BRRN1(23397); CCNT2(905); SLC39A10(57181); RGPD5 |
| | | (84220); IGFBP5(3488); OXNAD1(92106); LIMD1(8994); SENP2(59343); |
| | | FHIT(2272); C3orf17(25871); PIK3CB(5291); CCNG2(901); SCYE1(9255); |
| | | RAD17(5884); C5orf16(285613); IRF1(3659); RIPK1(8737); PTP4A1 |
| | | (7803); TNFAIP3(7128); MDC1(9656); BCLAF1(9774); CYCS(54205); PPP1R3B |
| | | (79660); TRIM35(23087); SEMA4D(10507); C9orf125(84302); FBXW2 |
| | | (26190); TRAF1(7185); PFKFB3(5209); CSPG6(9126); RP11-142I17.1 |
| | | (26095); MRPL43(84545); DCLRE1A(9937); C10orf46(143384); MKI67(4288); |
| | | TRIM6(117854); CAPN5(726); BIRC2(329); EIF4G2(1982); CDCA5 |
| | | (113130); FOXJ2(55810); STK38L(23012); TEGT(7009); MDM2(4193); ITPR2 |
| | | (3709); MCRS1(10445); FAIM2(23017); ZBTB39(9880); DUSP6(1848); |
| | | C13orf6(84945); BCL2L2(599); KIAA0323(23351); TXNDC(81542); ACIN1 |
| | | (22985); AKT1(207); TLN2(83660); MYO5A(4644); FLJ22795(80154); TSC2 |
| | | (7249); PKMYT1(9088); PIK3R5(23533); NUFIP2(57532); ERN1(2081); |
| | | CIDEA(1149); AXL(558); PPP2R1A(5518); NALP2(55655); FLJ38288(284309); |
| | | TRIB3(57761); CABLES2(81928); DIDO1(11083); HUNK(30811); |
| | | MAPK1(5594); PDCD8(9131); GABRE(2564); |
| hsa-miR-493-3p | 2061; 2125; | PRKCZ(5590); TP73(7161); TXLNA(200081); LCK(3932); FLJ25476(149076); |
| | 2317; 2318; | MACF1(23499); ZNF691(51058); PROK1(84432); PEA15(8682); UHMK1 |
| | | (127933); C1orf21(81563); ADORA1(134); TRAF5(7188); C1orf95(375057); |
| | | TNFRSF18(8784); CDC2L2(985); TNFRSF14(8764); FRAP1(2475); |
| | | CASP9(842); RCC2(55920); CLSPN(63967); TESK2(10420); GFI1(2672); |
| | | SASS6(163786); CTSS(1520); ADAR(103); ASPM(259266); RBBP5(5929); |
| | | C1orf198(84886); GREB1(9687); FOSL2(2355); PPP1CB(5500); ANTXR1 |
| | | (84168); HK2(3099); MAL(4118); RIF1(55183); CFLAR(8837); CD28(940); |
| | | ALS2CR19(117583); HRB(3267); SEPT2(4735); EIF2AK2(5610); TIA1(7072); |
| | | TGFA(7039); ANAPC1(64682); IL1B(3553); BIN1(274); ERCC3(2071); |
| | | FLJ34870(401013); STAT1(6772); CLK1(1195); CUL3(8452); TDGF1(6997); |
| | | RBM5(10181); IL17RB(55540); MCM2(4171); CHRD(8646); OPA1(4976); |
| | | RAF1(5894); SLC6A20(54716); TMEM110(375346); SLC12A8(84561); |
| | | TFDP2(7029); SEPT11(55752); DKFZp761B107(91050); APBB2(323); RASSF6 |
| | | (166824); MAD2L1(4085); NEK1(4750); NIPBL(25836); MRPS30 |
| | | (10884); CDX1(1044); LSM11(134353); CPLX2(10814); UNC5A(90249); PRLR |
| | | (5618); PRKAA1(5562); PLCXD3(345557); UNG2(10309); BIRC1(4671); |
| | | RPS23(6228); FLJ11292(55338); MCC(4163); FGF1(2246); MGC21644 |
| | | (153768); ADAMTS2(9509); RIPK1(8737); LTA(4049); CDKN1A(1026); |
| | | VEGF(7422); CDC5L(988); FOXO3A(2309); SASH1(23328); TXNDC5(81567); |
| | | HERV-FRD(405754); NEDD9(4739); MDC1(9656); CUL7(9820); MCM3 |
| | | (4172); DST(667); ATG5(9474); BCLAF1(9774); PLAGL1(5325); KATNA1 |
| | | (11104); ADCY1(107); MUC17(140453); HBP1(26959); PMS2(5395); SOSTDC1 |
| | | (25928); YWHAG(7532); CDK6(1021); POLR3D(661); BNIP3L(665); |
| | | PTK2B(2185); UNC5D(137970); TACC1(6867); SULF1(23213); MYC |
| | | (4609); BIN3(55909); TNFRSF10B(8795); TNFRSF10A(8797); ADRA1A(148); |
| | | DMRT2(10655); JAK2(3717); GADD45G(10912); INVS(27130); RXRA |
| | | (6256); TAF1L(138474); BAG1(573); GNE(10020); GAS1(2619); SEMA4D |
| | | (10507); C9orf21(195827); PPP6C(5537); TSC1(7248); DKK1(22943); SGPL1 |
| | | (8879); BTRC(8945); CASC2(255082); DOCK1(1793); ADARB2(105 |

Table 4-88

| | | |
|---|---|---|
| | | ); PARD3(56288); CUL2(8453); ZWINT(11130); KCNMA1(3778); SMNDC1 |
| | | (10285); C10orf119(79892); STIM1(6786); WEE1(7465); API5(8539); F2 |
| | | (2147); SIPA1(6494); RPS6KB2(6199); CCND1(595); ATM(472); TAGLN(6876); |
| | | MIZF(25988); CBL(867); LOH11CR2A(4013); C11orf37(440072); MUCDHL |
| | | (53841); CDKN1C(1028); APBB1(322); EIF4G2(1982); DKK3(27122); |
| | | SYT7(9066); BAD(572); RELA(5970); CLCF1(23529); PPP1CA(5499); |
| | | FZD4(8322); IL18(3606); CCND2(894); PTPN6(5777); ATF7IP(55729); |
| | | NCKAP1L(3071); PDE1B(5153); CCT2(10576); APAF1(317); RAN(5901); |
| | | KRAS(3845); MCRS1(10445); BTG1(694); IGF1(3479); ANAPC7(51434); |
| | | FBXL10(84678); TNFRSF19(55504); BRCA2(675); RFP2(10206); CDC16 |
| | | (8881); XPO4(64328); LATS2(26524); CSNK1A1L(122011); LTB4R2(56413); |
| | | CGRRF1(10668); PSEN1(5663); DAD1(1603); GPR68(8111); LOC440295 |
| | | (440295); UBE3A(7337); BMF(90427); BCL2L10(10017); PYGO1(26108); |
| | | DAPK2(23604); BCL2A1(597); RAB40C(57799); TSC2(7249); TRAF7(84231); |
| | | TNFRSF12A(51330); MGC13024(93129); CTCF(10664); CDK10(8558); |
| | | TXNDC11(51061); BOLA2(552900); N4BP1(9683); DOK4(55715); SLC12A4 |
| | | (6560); TERF2(7014); TXNL4B(54957); GSG2(83903); ZNF286(57335); |
| | | MAPK7(5598); TRAF4(9618); FLJ35848(284071); MAPT(4137); NGFR |
| | | (4804); TLK2(11011); PRKCA(5578); SLC43A2(124935); GAS7(8522); |
| | | SPAG5(10615); PHB(5245); ERN1(2081); SOCS3(9021); CSNK1D(1453); |
| | | CIDEA(1149); CABLES1(91768); CCDC5(115106); C18orf25(147339); PPP4R1 |
| | | (9989); ROCK1(6093); CSNK1G2(1455); RAB8A(4218); FLJ20850 |
| | | (55049); SAMD4B(55095); MAPSK10(4294); AXL(558); NALP2(55655); DAPK3 |
| | | (1613); TNFSF7(970); CDC37(11140); DEDD2(162989); LOC352909 |
| | | (352909); TTLL9(164395); STK4(6789); CD40(958); RAB22A(57403); TBC1D20 |
| | | (128637); E2F1(1869); CABLES2(81928); DIDO1(11083); MX1(4599); |
| | | PDXK(8566); SNTF1LK(150094); ITGB2(3689); BCL2L13(23786); CDC45L |
| | | (8318); SMARCB1(6598); ADORA2A(135); CSF2RB(1439); KDELR3 |
| | | (11015); PPARA(5465); GTSE1(51512); MAPK1(5594); PPM1F(9647); MN1 |
| | | (4330); RP11-191L9.1(400932); FLJ41993(400935); MAPK12(6300); TAF1 |
| | | (6872); AGTR2(186); PPP2R3B(28227); SMC1L1(8243); SPIN2(54466); |
| | | RNF12(51132); BTK(695); SEPT6(23157); HCFC1(3054); |
| hsa-miR-532 | 2062; 2119; | TNFRSF1B(7133); VPS13D(55187); KIAA1026(23254); CDC42(998); LCK |
| | 2319; 2320; | (3932); RBBP4(5928); CDC20(991); ST6GALNAC5(81849); CDC14A(8556); |
| | | SLC30A7(148867); RAP1A(5906); SYCP1(6847); PTGFRN(5738); RBM8A |
| | | (9939); BCL9(607); KIAA0460(23248); SLAMF8(56833); SLAMF7(57823); |
| | | FAM20B(9917); C1orf21(81563); RASSF5(83593); IL19(29949); C1orf69 |
| | | (200205); DISC1(27185); RERE(473); DFFA(1676); FRAP1(2475); E2F2 |
| | | (1870); TTMB(399474); YARS(8565); SF3A3(10946); TESK2(10420); |
| | | C1orf123(54987); DHCR24(1718); RBMXL1(494115); GBP1(2633); GFI1 |
| | | (2672); ISG20L2(81875); SYT2(127833); RABIF(5877); IL10(3586); TP53BP2 |
| | | (7159); ABCB10(23456); RHOB(388); BIRC6(57448); MSH2(4436); CCT7 |
| | | (10574); HTRA2(27429); HK2(3099); MGC52057(130574); RIF1(55183); |
| | | FLJ13096(80067); UBE2E3(10477); CFLAR(8837); CASP10(843); FZD7 |
| | | (8324); C2orf21(285175); RHBDD1(84236); COL4A3(1285); PSMD1(5707); |
| | | SLC8A1(6546); TIA1(7072); C2orf3(6936); EDAR(10913); ANAPC1(64682); |
| | | IL1A(3552); FLJ10996(54520); MCM6(4175); ARL5A(26225); KIAA1715 |
| | | (80856); SESTD1(91404); STAT1(6772); BARD1(580); VHL(7428); SYN2 |
| | | (6854); NR2C2(7182); PCAF(8850); FBXL2(25827); PDCD6IP(10015); |
| | | TDGF1(6997); RBM5(10181); APPL(26060); PTPRG(5793); NEK11(79858); |
| | | NUDT16(131870); UBE1DC1(79876); SMC4L1(10051); LPP(4028); OPA1 |
| | | (4976); TADA3L(10474); IL17RD(54756); FLJ34969(201627); FOXP1 |
| | | (27086); PROK2(60675); FLJ44076(401080); DOC1(11259); MAK3(80218); |
| | | PTPLB(201562); OSBPL11(114885); ATR(545); EREG(2069); SEPT11(55752); |
| | | FGF5(2250); C4orf12(404201); NFKB1(4790); NPNT(255743); SEC24B |
| | | (10427); FGF2(2247); TLR2(7097); TLL1(7092); PALLD(23022); STOX2 |
| | | (56977); AFAP(60312); APBB2(323); IGFBP7(3490); RASSF6(166824); |
| | | ANAPC10(10393); MFAP3L(9848); CARD6(84674); LOC285636(285636); |
| | | ERBB2IP(55914); F2R(2149); APC(324); RAD50(10111); PCDHB9(56127); |
| | | FGF10(2255); IL6ST(3572); TMED7(51014); ATG12(9140); CSNK1A1 |
| | | (1452); NMUR2(56923); STC2(8614); DDX41(51428); FLT4(2324); RIPK1 |
| | | (8737); SIRT5(23408); E2F3(1871); TCF19(6941); SCUBE3(222663); BAG2 |
| | | (9532); SH3BGRL2(83699); RP5-875H10.1(389432); SASH1(23328); |
| | | UST(10090); ULBP1(80329); RP11-145H9.1(340156); NEDD9(4739); DHX16 |
| | | (8449); SUPT3H(8464); MCM3(4172); TRAM2(9697); DST(667); MAP |

Table 4-89

| | | |
|---|---|---|
| | | 3K5(4217); PLAGL1(5325); FLJ44955(401278); PDCD2(5134); ABCA13 |
| | | (154664); ADAM22(53616); ZKSCAN1(7586); STAG3(10734); MUC17(140453); |
| | | MDFIC(29969); CYCS(54205); SFRP4(6424); CALN1(83698); CDK6 |
| | | (1021); PDK4(5166); FIS1(51024); PPP1R3A(5506); TFEC(22797); WNT2 |
| | | (7472); FZD3(7976); UNC5D(137970); DDHD2(23259); TACC1(6867); SULF1 |
| | | (23213); MTDH(92140); FZD6(8323); TNFRSF10B(8795); TNFRSF10D |
| | | (8793); TNFRSF10A(8797); TRIM35(23087); BRF2(55290); FGFR1(2260); |
| | | ZNF704(619279); MMP16(4325); SLC25A32(81034); EXT1(2131); TNFRSF11B |
| | | (4982); MTSS1(9788); JRK(8629); GCNT1(2650); DAPK1(1612); |
| | | FCMD(2218); C9orf6(54942); TRAF2(7186); BNC2(54796); BAG1(573); ZBTB5 |
| | | (9925); TXNDC4(23071); EPB41L4B(54566); TSC1(7248); ZMYND11 |
| | | (10771); HSPA14(51182); EIF4EBP2(1979); SGPL1(8879); PTEN(5728); |
| | | PCGF5(84333); HECTD2(143279); BTRC(8945); NOLC1(9221); SUFU |
| | | (51684); CSPG6(9126); KIAA1600(57700); AKR1C2(1646); NUDT5(11164); |
| | | NEBL(10529); PARD3(56288); PLEKHK1(219790); KCNMA1(3778); ANKRD1 |
| | | (27063); CPEB3(22849); TLL2(7093); COX15(1355); SH3PXD2A |
| | | (9644); DCLRE1A(9937); MKI67(4288); STIM1(6786); MS4A2(2206); DAK |
| | | (26007); SIPA1(6494); BIRC2(329); DDI1(414301); CUL5(8065); ATM(472); |
| | | LOC399947(399947); STS-1(84959); SIRT3(23410); WT1(7490); CASP1 |
| | | (834); FLJ22028(79912); SURB7(9412); ESPL1(9700); DYRK2(8445); CCT2 |
| | | (10576); GAS2L3(283431); KIAA1033(23325); KNTC1(9735); LRP6(4040); |
| | | BCAT1(586); KRAS(3845); TMTC1(83857); IPO8(10526); SYT10(341359); |
| | | FAIM2(23017); KRT1B(374454); DDIT3(1649); CDK4(1019); WIF1 |
| | | (11197); TMCC3(57458); HRK(8739); C12orf43(64897); SCARB1(949); CHFR |
| | | (55743); HSMPP8(54737); FGF9(2254); CDK8(1024); BRCA2(675); HS6ST3 |
| | | (266722); RAP2A(5911); CUL4A(8451); FOXO1A(2308); LIG4(3981); |
| | | MIPOL1(145282); NRXN3(9369); FLRT2(23768); DAD1(1603); STRN3(29966); |
| | | EXOC5(10640); SYNJ2BP(55333); CHES1(1112); BTBD7(55727); |
| | | BAG5(9529); RAB11A(8766); KIF23(9493); PML(5371); C15orf37(283687); |
| | | AVEN(57099); PYGO1(26108); CSNK1G1(53944); DNAJA3(9093); MKL2 |
| | | (57496); CYLD(1540); DNAJA2(10294); SIAH1(6477); AMFR(267); LOC283849 |
| | | (283849); MAF(4094); RPA1(6117); GSG2(83903); CPD(1362); RHBDL3 |
| | | (162494); PPM1D(8493); KPNA2(3838); HCP1(113235); SSH2(85464); |
| | | GAS2L2(246176); RPL23(9349); BRCA1(672); LOC641522(641522); |
| | | TBC1D16(125058); SGSH(6448); CABLES1(91768); MALT1(10892); PHLPP |
| | | (23239); SPIRE1(56907); ROCK1(6093); DSC3(1825); FAM59A(64762); |
| | | SMAD2(4087); FZR1(51343); DNM2(1785); CASP14(235181); RAB8A(4218); |
| | | APOE(348); ZNF480(147657); TNFSF7(970); ZNF443(10224); PDE4C |
| | | (5143); LIG1(3978); CARD8(22900); KLK10(5655); MCM8(84515); PLCB1 |
| | | (23236); CD40(958); VAPB(9217); PTPRT(11122); ATP9A(10079); SYCP2 |
| | | (10388); GABPA(2551); HUNK(30811); SON(6651); SLC5A3(6526); APP |
| | | (351); SEPT5(5413); LOC284861(284861); EP300(2033); GTSE1(51512); |
| | | MAPK1(5594); PPM1F(9647); CHEK2(11200); C22orf23(84645); C22orf9 |
| | | (23313); SMC1L2(27127); MAPK12(6300); STS(412); XK(7504); TAF1 |
| | | (6872); AGTR2(186); BIRC4(331); CD40LG(959); AFF2(2334); SPRY3(10251); |
| | | RPS6KA3(6197); TAB3(257397); SMC1L1(8243); RP11-130N24.1(340533); |
| | | DCX(1641); AMOT(154796); SEPT6(23157); CXorf48(54967); ZNF75 |
| | | (7626); MECP2(4204); |
| hsa-miR-542-3p | 2063; 2120; | PRDM16(63976); TP73(7161); MFN2(9927); FLJ43806(399563); C1orf130 |
| | 2121; | (400746); RHD(6007); SEPN1(57190); RCC1(1104); MACF1(23499); CDC20 |
| | | (991); CDKN2C(1031); SH3GLB1(51100); RTCD1(8634); RAP1A(5906); |
| | | SYCP1(6847); PEA15(8682); UHMK1(127933); C1orf76(148753); PPP1R12B |
| | | (4660); MDM4(4194); SRGAP2(23380); TRIM58(25893); TNFRSF14 |
| | | (8764); TNFRSF9(3604); AKR7A2(8574); C1orf166(79594); FABP3(2170); |
| | | LOC113444(113444); KIAA0319L(79932); CLSPN(63967); PPT1(5538); |
| | | SERBP1(26135); EVI5(7813); CLCC1(23155); PYGO2(90780); CD5L(922); |
| | | GLT25D2(23127); PPP1R15B(84919); ENAH(55740); FH(2271); RHOB |
| | | (388); PPP1CB(5500); YPEL5(51646); BIRC6(57448); RHOQ(23433); SPTBN1 |
| | | (6711); HK2(3099); TCF7L1(83439); LOC400965(400965); ZNF2(7549); |
| | | SCN2A2(6326); MYO3B(140469); ZAK(51776); CFLAR(8837); CASP10 |
| | | (843); FZD7(8324); NRP2(8828); RPE(6120); INHA(3623); COL4A3(1285); |
| | | FLJ43374(377007); GPC1(2817); SEPT2(4735); BOK(666); ING5(84289); |
| | | PDIA6(10130); PPM1G(5496); SUPT7L(9913); TMEM127(55654); LMAN2L |
| | | (81562); ST6GAL2(84620); EDAR(10913); WDR33(55339); ARL5A |
| | | (26225); CD302(9936); STAT1(6772); PGAP1(80055); LANCL1(10314); |

Table 4-90

ERBB4(2066); IGFBP5(3488); PAX3(5077); EDEM1(9695); ITGA9(3680);
ZNF621(285268); TDGF1(6997); TREX1(11277); GLYCTK(132158); IL17RB
(55540); APPL(26060); SIDT1(54847); GTF2E1(2960); ARGFX(503582);
NEK11(79858); FAIM(55179); CHRD(8646); LPP(4026); IL1RAP(3556);
RAD18(56852); TADA3L(10474); ZFYVE20(64145); RAFTLIN(23180);
C3orf48(151649); UBP1(7342); SCN5A(6331); AXUD1(64651); RASSF1(11186);
DNASE1L3(1776); AER61(285203); PROK2(60675); SLC12A8(84561);
STAG1(10274); PIK3CB(5291); USH3A(7401); PPP1R2(5504); PCYT1A
(5130); WHSC1(7468); HD(3064); CCNG2(901); FGF5(2250); TLR2(7097);
LETM1(3954); MLR1(254251); APBB2(323); GRSF1(2926); UNC5C
(8633); MFAP3L(9848); SKP2(6502); LOC493869(493869); ERBB2IP(55914);
PIK3R1(5295); MAP1B(4131); RAD50(10111); PCDHB13(56123); PCDHGB4
(8641); TPPP(11076); DAP(1611); PRLR(5618); FGF10(2255); BIRC1
(4671); ZBED3(84327); ARSB(411); FLJ25680(134187); LIX1(167410);
MCC(4163); SEMA6A(57556); IRF1(3659); PPP2CA(5515); CDC23(8697);
SPRY4(81848); KIAA1893(114787); LMAN2(10960); PRSS16(10279); TNF
(7124); SCUBE3(222663); MAPK13(5603); CDKN1A(1026); PARC(23113);
SLC22A7(10864); CDC5L(988); SH3BGRL2(83699); TTK(7272); FOXO3A
(2309); KIAA1919(91749); TNFAIP3(7128); SASH1(23328); ESR1
(2099); QKI(9444); ATXN1(6310); DHX16(8449); MDC1(9656); IER3(8870);
CUL7(9820); MCM3(4172); DST(667); HBS1L(10767); PLAGL1(5325); LATS1
(9113); TFB1M(51106); LPAL2(80350); FRMD1(79981); MAFK(7975);
FOXK1(221937); AHR(196); CREB5(9586); DKFZp761I2123(83637); ADCY1
(107); ABCA13(154664); EGFR(1956); CASP2(835); NOM1(64434);
UBE3C(9690); TRIAD3(54476); PMS2(5395); HUS1(3364); PMS2L2(5380);
CDK6(1021); FLJ36031(168455); TFEC(22797); PTN(5764); SCARA3
(51435); UNC5D(137970); TACC1(6867); CA13(377677); RIPK2(8767); C8orf1
(734); FLJ45248(401472); ATP6V1C1(528); ZNF623(9831); XKR5(389610);
LZTS1(11178); TNFRSF10B(8795); ADRA1A(148); SFRP1(6422); ZNF704
(619279); TNFRSF11B(4982); TMEM65(157378); ST3GAL1(6482);
C9orf26(90865); RECK(8434); DAPK1(1612); KIAA1958(158405); RALGPS1
(9649); CDK9(1025); FAM73B(84895); ABL1(25); TRAF2(7186); PTGDS
(5730); TAF1L(138474); BAG1(573); FANCG(2189); C9orf100(84904);
PTCH(5727); EPB41L4B(54566); TNFSF8(944); ASB6(140459); TSC1(7248);
HSPA14(51182); SIRT1(23411); SGPL1(8879); UNC5B(219699); CHST3
(9469); SFTPA1(6435); OPN4(94233); PTEN(5728); CYP2C9(1559); LZTS2
(84445); NOLC1(9221); PDCD11(22984); GPR26(2849); ADARB2(105);
NUDT5(11164); PAPD1(55149); FZD8(8325); ZWINT(11130); SFTPA2
(6436); FAM26C(255022); SH3PXD2A(9644); KIAA1754(85450); C10orf46
(143384); MKI67(4288); STIM1(6786); HIPK3(10114); LDLRAD3(143458);
F2(2147); INCENP(3619); URP2(83706); SIPA1(6494); SSH3(54961);
CTTN(2017); LRRC51(220074); BIRC2(329); ATM(472); SNF1LK2(23235);
TAGLN(6876); CD3E(916); LOH11CR2A(4013); APLP2(334); OR51E2
(81285); EIF4G2(1982); LOC221091(221091); ZBTB3(79842); MAP3K11
(4296); PPP1CA(5499); PTPRCAP(5790); PAK1(5058); FZD4(8322); GPR83
(10888); BCL9L(283149); SCN3B(55800); OPCML(4978); CCND2(894);
ESPL1(9700); CDK2(1017); PA2G4(5036); RBMS2(5939); MON2(23041);
APAF1(317); KIAA1853(84530); TCF1(6927); ATP6V0A2(23545); FGF6(2251);
TNFRSF1A(7132); NOL1(4839); BCAT1(586); ZNF641(121274); C12orf22
(81566); CBX5(23468); GLS2(27165); R3HDM2(22864); LRRC10(376132);
ANAPC7(51434); HRK(8739); ATP8A2(51761); WASF3(10810); RFP2
(10206); PCDH17(27253); ATP11A(23250); LATS2(26524); TEB1(54937);
FOXO1A(2308); ATP7B(540); KCTD12(115207); FLJ10154(55082); LOC283487
(283487); RNF31(55072); LTB4R2(56413); SAMD4A(23034); C14orf111
(51077); C14orf118(55668); OTUB2(78990); TRAF3(7187); KNS2
(3831); JUB(84962); CIDEB(27141); STRN3(29966); POMT2(29954); FBLN5
(10516); MOAP1(64112); AKT1(207); JAG2(3714); CHP(11261); FGF7
(2252); Gcom1(145781); PML(5371); SCAND2(54581); NGRN(51335); ST8SIA2
(8128); LOC400464(400464); BMF(90427); EHD4(30844); GNB5
(10681); MYO5A(4644); ALDH1A2(8854); BNIP2(663); PDCD7(10081); FLJ22795
(80154); LOC400451(400451); LYSMD4(145748); LOC440313(440313);
KIAA1924(197335); CACNA1H(8912); KREMEN2(79412); KIAA0350
(23274); MKL2(57496); EEF2K(29904); TNT(162083); SPN(6693); PRRT2
(112476); RBL2(5934); CCL22(6367); CTCF(10664); CMIP(80790); CDH13
(1012); FLJ20186(54949); AXIN1(8312); NME3(4832); GSPT1(293

Table 4-91

| | | |
|---|---|---|
| | | 5); ERN2(10595); MAPK3(5595); FTS(64400); ZNF319(57567); TERF2(7014); |
| | | TXNL4B(54957); PCOLN3(5119); ALOX15B(247); PFAS(5198); NLK |
| | | (51701); NF1(4763); LASP1(3927); STARD3(10948); CSF3(1440); CNTD(124817); |
| | | FZD2(2535); MAPT(4137); PNPO(55163); CALCOCO2(10241); PPM1D |
| | | (8493); KPNA2(3838); MAP2K6(5608); KCTD2(23510); SPHK1(8877); |
| | | BIRC5(332); ZZEF1(23140); UBE2G1(7326); AURKB(9212); GAS7(8522); |
| | | LOC116236(116236); TMKM101(84336); HDAC5(10014); SCRN2(90507); |
| | | PHB(5245); RNF43(54894); LIMD2(80774); LYK5(92335); ERN1(2081); |
| | | RECQL5(9400); TRIM65(201292); MXRA7(439921); PCYT2(5833); |
| | | CABLES1(91768); DCC(1630); SERPINB8(5271); BCL2(596); ZADH2(284273); |
| | | FZR1(51343); VMAC(400673); DNM2(1785); UBA52(7311); FLJ20850 |
| | | (55049); MGC35440(147990); APLP1(333); C19orf15(57828); SAMD4B |
| | | (55095); CYP2B6(1555); PVR(5817); APOE(348); HIF3A(64344); PPP2R1A |
| | | (5518); NALP2(55655); ZNF264(9422); ADAMTSL5(339366); TNFSF14 |
| | | (8740); RAB3D(9545); HAPLN4(404037); KIAA1559(57677); DPF1(8193); |
| | | SIRT2(22933); FLJ41131(284325); DEDD2(162989); FLJ10781(55228); |
| | | LOC284296(284296); ZNF497(162968); TRIB3(57761); STK35(140901); |
| | | KIF3B(9371); ASXL1(171023); MMP24(10893); CEP250(11190); C20orf102 |
| | | (128434); MYBL2(4605); C20orf35(55861); PARD6B(84612); C20orf11 |
| | | (54994); NSFL1C(55968); PCNA(5111); E2F1(1869); GGTL3(2686); DIDO1 |
| | | (11083); IFNAR1(3454); SON(6651); APP(351); ZNF295(49854); SNF1LK |
| | | (150094); ICOSLG(23308); ITGB2(3689); LSS(4047); DGCR6(8214); LZTR1 |
| | | (8216); RAB36(9609); ADORA2A(135); NF2(4771); MCM5(4174); APOBEC3F |
| | | (200316); SREBF2(6721); SEPT3(55964); hCAP-H2(29781); CECR6 |
| | | (27439); MAPK1(5594); CHEK2(11200); DUSP18(150290); MYH9(4627); |
| | | TOB2(10766); NHP2L1(4809); PDHA1(5160); HDAC6(10013); YIPF6 |
| | | (286451); SLC16A2(6567); BHLHB9(80823); TKTL1(8277); PPP2R3B(28227); |
| | | KAL1(3730); FIGF(2277); RNF12(51132); DCX(1641); CUL4B(8450); |
| | | GABRE(2564); HCFC1(3054); MECP2(4204); |
| hsa-miR-545 | 2064; | H6PD(9563); KIF1B(23095); PQLC2(54896); CDC42(998); SESN2(83667); |
| | | RBBP4(5928); EIF2C4(192670); MACF1(23499); CDC20(991); CDKN2C |
| | | (1031); C1orf83(127428); BXDC5(80135); RTCD1(8634); CDC14A(8556); |
| | | RAP1A(5906); SYCP1(6847); VANGL1(81839); BCL9(607); CKS1B(1163); |
| | | UHMK1(127933); DDR2(4921); DNM3(26052); C1orf76(148753); IL24(11009); |
| | | TGFB2(7042); LGALS8(3964); MTR(4548); CIAS1(114548); GNB1(2782); |
| | | TNFRSF14(8764); DNAJC11(55735); CTNNBIP1(56998); FRAP1 |
| | | (2475); ZBTB17(7709); RCC2(55920); YARS(8565); KIAA0319L(79932); CLSPN |
| | | (63967); HPCAL4(51440); BMP8B(656); MUTYH(4595); SPATA6(54558); |
| | | FAF1(11124); GBP4(115361); IGSF3(3321); NOTCH2(4853); MCL1 |
| | | (4170); PIK4CB(5298); FCRL5(83416); DEDD(9191); SH2D1B(117157); NMNAT2 |
| | | (23057); TNNI1(7135); LPGAT1(9926); TP53BP2(7159); PGBD5 |
| | | (79605); RAB10(10890); PPP1CB(5500); YIPF4(84272); BIRC6(5744B); MSH2 |
| | | (4436); SLC1A4(6509); FLJ16124(440867); CCT7(10574); HTRA2(27429); |
| | | HK2(3099); MRPL19(9801); BRRN1(23397); BCL2L11(10018); STEAP3 |
| | | (55240); CCNT2(905); RIF1(55183); FMNL2(114793); TAIP-2(80034); |
| | | LASS6(253782); ZNF650(130507); FLJ13096(80067); ZAK(51776); CASP8 |
| | | (841); BMPR2(659); ABI2(10152); PTMA(5757); YWHAQ(10971); CGREF1 |
| | | (10669); CARD12(58484); EIF2AK2(5610); CCT4(10575); TIA1(7072); |
| | | TGFA(7039); REEP1(65055); ANAPC1(64682); IL1A(3552); ERCC3(2071); |
| | | WDR33(55339); IFIH1(64135); NCKAP1(10787); STAT1(6772); CUL3 |
| | | (8452); TRIP12(9320); OXNAD1(92106); CRTAP(10491); MLH1(4292); ZNF621 |
| | | (285268); CTNNB1(1499); APPL(26060); PXK(54899); EPHA3(2042); |
| | | PCNP(57092); KALRN(8997); MCM2(4171); NEK11(79858); ACPP(55); |
| | | SMC4L1(10051); FXR1(8087); TP73L(8626); OPA1(4976); TADA3L(10474); |
| | | KIAA1143(57456); NME6(10201); QRICH1(54870); UBE1C(9039); EIF4E3 |
| | | (317649); RABL3(285282); MYLK(4638); STAG1(10274); PIK3CB(5291); |
| | | ATR(545); P2RY13(53829); TNFSF10(8743); PCYT1A(5130); WHSC1 |
| | | (7468); HD(3064); TBC1D14(57533); CD38(952); HCAP-G(64151); ANAPC4 |
| | | (29945); MOBKL1A(92597); EREG(2069); SEPT11(55752); FGF5(2250); |
| | | NFKB1(4790); FLJ20032(54790); FGF2(2247); PLK4(10733); GAK(2580); |
| | | LRPAP1(4043); APBB2(323); PRKG2(5593); UNC5C(8633); EIF4E |
| | | (1977); DKK2(27123); LEF1(51176); CAMK2D(817); C4orf18(51313); CBR4 |
| | | (84869); NEK1(4750); SORBS2(8470); NIPBL(25836); ITGA1(3672); PIK3R1 |
| | | (5295); PTCD2(79810); LOC90355(90355); APC(324); FLJ90650(206338); |
| | | CSNK1G3(1456); GRAMD3(65983); RAD50(10111); PCDHB9(561 |

Table 4-92

27); ARHGAP26(23092); G3BP(10146); UNC5A(90249); PRLR(5618); PRKAA1
(5562); UNG2(10309); MCC(4163); ALDH7A1(501); CDC23(8697);
CDC25C(995); HDAC3(8841); CSNK1A1(1452); ADAM19(8728); DUSP1
(1843); RNF130(55819); IRF4(3662); PRPF4B(8899); E2F3(1871); HDGFL1
(154150); TNF(7124); CSNK2B(1460); LOC401252(401252); NFYA(4800);
PARC(23113); C6orf65(221336); COL19A1(1310); TTK(7272); FOXO3A
(2309); KIAA1919(91749); NEDD9(4739); DHX16(8449); MDC1(9656);
DAXX(1616); IHPK3(117283); TRFP(9477); CUL7(9820); SUPT3H(8464);
TNFRSF21(27242); FLJ41841(401263); PKHD1(5314); DST(667); CCNC
(892); FRK(2444); PTPRK(5796); PLAGL1(5325); LATS1(9113); AGPAT4
(56895); PDE10A(10846); PHF14(9678); ARL4A(10124); ZNRF2(223082);
SEPT7(989); EGFR(1956); LOC441257(441257); STAG3(10734); RINT1
(60561); BCAP29(55973); ASB15(142685); TRIM24(8805); LUC7L2(51631);
MAD1L1(8379); TRIAD3(54476); JAZF1(221895); CARD4(10392); INHBA
3624); TMED4(222068); H2AFV(94239); PURB(5814); CALCR(799);
MCM7(4176); PODXL(5420); ZC3HAV1(56829); KBTBD11(9920); UNC5D
(137970); VPS13B(157680); FZD6(8323); WISP1(8840); GML(2765); EGR3
(1960); DUSP4(1846); GTF2E2(2961); SFRP1(6422); TP53INP1(94241);
EDD1(51366); CYP11B1(1584); CDC37L1(55664); UNC13B(10497); RECK
(8434); OSTF1(26578); VPS13A(23230); NTRK2(4915); DAPK1(1612);
C9orf47(286223); TGFBR1(7046); INVS(27130); OLFML2A(169611); USP20
(10868); ABL1(25); RLN2(6019); CDEN2B(1030); DDX58(23586); FANCG
(2189); RNF38(152006); KIAA0367(23273); SEMA4D(10507); IPPK
(64768); PTCH(5727); ROD1(9991); TRAF1(7185); TOR2A(27433); CARD9
(64170); ANAPC2(29882); CUGBP2(10659); ARL5B(221079); CDC2(983);
SIRT1(23411); KIAA1274(27143); FAS(355); TMEM20(159371); PI4KII
(55361); ENTPD7(57089); HIF1AN(55662); BTRC(8945); SLK(9748); PNLIPRP3
(119548); SEC23IP(11196); TACC2(10579); ACADSB(36); DOCK1
(1793); VENTX(27287); CUL2(8453); DNAJC12(56521); DLG5(9231); HTR7
(3363); TMEM10(93377); PIK3AP1(118788); ARHGAP19(84986); SMNDC1
(10285); DCLRE1A(9937); VAX1(11023); MKI67(4288); HIPK3(10114);
SPCS2(9789); C11orf54(28970); CUL5(8065); ATM(472); IL10RA(3587);
CD3G(917); EI24(9538); BET1L(51272); NUP98(4928); RHOG(391); XLKD1
(10894); EIF4G2(1982); CSRP3(8048); BRMS1(25855); PGM2L1(283209);
RAB30(27314); FZD4(8322); SESN3(143686); PDGFD(80310); ARHGAP20
(57569); PPP2R1B(5519); IL18(3606); ZW10(9183); PRDM10(56980);
IAPP(3375); ETNK1(55500); STK38L(23012); KLHDC5(57542); FGD4
(121512); GPD1(2819); PDE1B(5153); CDK2(1017); C12orf28(196446);
GAS2L3(283431); KNTC1(9735); ZNF10(7556); RAD52(5893); BCAT1
(586); DBX2(440097); FAIM2(23017); C12orf22(81566); SLC41A2(84102);
ANAPC7(51434); ANAPC5(51433); SCARB1(949); HSMPP8(54737); TNFRSF19
(55504); BRCA2(675); UFM1(51569); RFP2(10206); SUGT1(10910);
TNFSF13B(10673); ING1(3621); ATP11A(23250); TFDP1(7027); XPO4
(64328); FLJ25477(219287); SLC7A1(6541); CSNK1A1L(122011); FOXO1A
(2308); FLJ10154(55082); LIG4(3981); RNF31(55072); PNN(5411); TXNDC
(81542); PELI2(57161); C14orf118(55668); VASH1(22846); KIAA1737
(85457); FLRT2(23768); GPR65(8477); TCL6(27004); TRAF3(7187); DAD1
(1603); GZMH(2999); EGLN3(112399); NFKBIA(4792); SOS2(6655); CDKL1
(8814); C14orf43(91748); NEK9(91754); CHES1(1112); GLDN(342035);
MAPK6(5597); FEM1B(10116); AR1H1(25820); HMG20A(10363); IREB2
(3658); ADAMTSL3(57188); SCAND2(54581); ALPK3(57538); ABHD2
(11057); AVEN(57099); BMF(90427); TTBK2(146057); MYEF2(50804);
BCL2L10(10017); ARPP-19(10776); CCPG1(9236); FLJ38723(255180);
EFTUD1(79631); FLJ22795(80154); ATF7IP2(80063); MKL2(57496); PLK1
(5347); IL4R(3566); CYLD(1540); RBL2(5934); E2F4(1874); CTCF(10664);
CYB5B(80777); ZNRF1(84937); FLJ11151(55313); NDUFAB1(4706);
ERN2(10595); MAPK3(5595); PLLP(51090); TRADD(8717); SMPD3(55512);
TXNL4B(54957); PAFAH1B1(5048); GSG2(83903); ALOX12(239); KCTD11
(147040); ALOX15B(247); MAPK7(5598); SARM1(23098); FLJ46247
(374786); CPD(1362); NF1(4763); TADA2L(6871); NXPH3(11248); RPS6KB1
(6198); PPM1D(8493); PRKCA(5578); SPHK1(8877); CARD14(79092);
GPS1(2873); NALP1(22861); AURKB(9212); FLCN(201163); FLJ36492
(284047); ULK2(9706); SPAG5(10615); NUFIP2(57532); AP1GBP1(11276);
BRCA1(672); HDAC5(10014); ZNF652(22834); RECQL5(9400); MXRA7
(439921); CSNK1D(1453); TGIF(7050); CABLES1(91768); SMAD4(4089)

Table 4-93

| | | |
|---|---|---|
| | | ; DOK6(220164); ESCO1(114799); DSC2(1824); PIGN(23556); PARD6G(84552); |
| | | CSNK1G2(1455); UHRF1(29128); ZNF333(84449); APOE(348); NALP2 |
| | | (55655); ZNF264(9422); TNFSF14(8740); PRDX2(7001); SLC35E1 |
| | | (79939); MGC2747(79086); C19orf12(83636); SIRT2(22933); DEDD2(162989); |
| | | ERCC2(2068); CARD8(22900); KLK10(5655); MCM8(84515); PLCB1 |
| | | (23236); SNAP25(6616); LPIN3(64900); STK4(6789); CD40(958); NCOA3 |
| | | (8202); CSE1L(1434); PARD6B(84612); RAB22A(57403); TUBB1(81027); |
| | | PCMTD2(55251); PCNA(5111); C20orf128(128876); SPATA2(9825); CTCFL |
| | | (140690); DIDO1(11083); HUNK(30811); SLC5A3(6526); BTG3(10950); |
| | | UBE2G2(7327); SUMO3(6612); ITGB2(3689); GNAZ(2781); NF2(4771); |
| | | APOL6(80830); SYNGR1(9145); RP5-1104E15.5(54471); TNRC6B(23112); |
| | | EP300(2033); GAB4(128954); YPEL1(29799); MN1(4330); DDX17(10521); |
| | | DMC1(11144); PDGFB(5155); CPT1B(1375); USP9X(8239); HDAC6(10013); |
| | | MAGEH1(28986); OGT(8473); SLC16A2(6567); RP11-321G1.1(54885); |
| | | GRIA3(2892); BIRC4(331); RP6-213H19.1(51765); DKC1(1736); PRKX |
| | | (5613); RGAG4(340526); RP11-130N24.1(340533); TAF9B(51616); LAMP2 |
| | | (3920); ELF4(2000); SUHW3(55609); HS6ST2(90161); ARHGEF6(9459); |
| | | FAM11A(84548); PRKY(5616); |
| hsa-miR-548a | 2065; | PRKCZ(5590); SESN2(83667); TXLNA(200081); RBBP4(5928); MACF1 |
| | | (23499); CDKN2C(1031); PRPF38A(84950); PRKAA2(5563); SH3GLB1(51100); |
| | | CDC7(8317); CDC14A(8556); RAP1A(5906); VANGL1(81839); SNX27 |
| | | (81609); DAP3(7818); PEA15(8682); UHMK1(127933); DDR2(4921); FAM20B |
| | | (9917); C1orf21(81563); ZC3H11A(9877); C1orf107(27042); TRAF5 |
| | | (7188); PROX1(5629); C1orf71(163882); CIAS1(114548); TNFRSF18(8784); |
| | | LUZP1(7798); CLSPN(63967); RIMS3(9783); FOXJ3(22887); SERBP1 |
| | | (26135); FUBP1(8880); TGFBR3(7049); GFI1(2872); EVI5(7813); TMED5 |
| | | (50999); SASS6(163786); NOTCH2(4853); MCL1(4170); MEF2D(4209); |
| | | ASPM(259266); IL10(3586); SLC30A1(7779); ENAH(55740); C1orf131(128061); |
| | | TOMM20(9804); RHOB(388); BIRC6(57448); EML4(27436); PRKCE |
| | | (5581); MSH2(4436); KCMF1(56888); KCNIP3(30818); BRRN1(23397); |
| | | RIF1(55183); TAIP-2(80034); ZAK(51776); ITGAV(3685); PMS1(5378); |
| | | NAB1(4664); CASP10(843); FZD7(8324); BMPR2(659); CREB1(1385); PSMD1 |
| | | (5707); SEPT2(4735); RBJ(51277); FLJ34931(388939); EIF2AK2(5610); |
| | | RTN4(57142); CCT4(10575); GFPT1(2673); TIA1(7072); AFF3(3899); |
| | | FLJ44006(400997); IL1A(3552); STAM2(10254); TLK1(9874); STAT1(6772); |
| | | PGAP1(80055); CUL3(8452); TRIP12(9320); HDAC4(9759); FANCD2 |
| | | (2177); VHL(7428); PDCD6IP(10015); TDGF1(6997); RBM5(10181); GNL3 |
| | | (26354); APPL(26060); KBTBD8(84541); MITF(4286); ZPLD1(131368); RNF7 |
| | | (9616); SMC4L1(10051); SKIL(6498); PIK3CA(5290); PSMD2(5708); |
| | | OPA1(4976); CLASP2(23122); UBE1C(9039); RYBP(23429); CD47(961); ZNF148 |
| | | (7707); STAG1(10274); PIK3CB(5291); XRN1(54464); SLAH2(6478); |
| | | TNFSF10(8743); TBL1XR1(79718); PCGF3(10336); HD(3064); HCAP-G |
| | | (64151); ANAPC4(29945); TMEM33(55161); REST(5978); SLC4A4(8671); |
| | | IL8(3576); EREG(2069); CCNG2(901); FGF5(2250); CYP2U1(113612); FGF2 |
| | | (2247); PLK4(10733); LARP2(55132); GAK(2580); HS3ST1(9957); GABRG1 |
| | | (2565); CORIN(10699); RCHY1(25898); PRKG2(5593); UBE2D3(7323); |
| | | NEK1(4750); VEGFC(7424); CASP3(836); SEP2(6502); NIPBL(25836); |
| | | CARD6(84674); ERBB2IP(55914); PIK3R1(5295); CCNB1(891); RAD17 |
| | | (5884); TNPO1(3842); APC(324); FLJ90650(206338); RAD50(10111); PCBD2 |
| | | (84105); SMAD5(4090); G3BP(10146); PRLR(5618); PLCXD3(345557); |
| | | PDE4D(5144); BIRC1(4671); ZBED3(84327); FLJ25680(134187); ST8SIA4 |
| | | (7903); MCC(4163); SEMA6A(57556); CDC23(8697); CDC25C(995); CSNK1A1 |
| | | (1452); RNF130(55819); VEGF(7422); BAG2(9532); PTP4A1(7803); |
| | | ANKRD6(22881); FOXO3A(2309); TNFAIP3(7128); SASH1(23328); WTAP |
| | | (9589); QKI(9444); ATXN1(6310); NUDT3(11165); SUPT3H(8464); DST |
| | | (667); SYNCRIP(10492); ATG5(9474); PDSS2(57107); FRK(2444); SGK |
| | | (6446); BCLAF1(9774); LATS1(9113); TFB1M(51106); PDE10A(10846); PDCD2 |
| | | (5134); AHR(196); HDAC9(9734); CREB5(9586); SEPT7(989); CDC2L5 |
| | | (8621); STK17A(9263); EGFR(1956); ADAM22(53616); BCAP29(55973); |
| | | PMS2(5395); DGKB(1607); CYCS(54205); ELMO1(9844); SFRP4(6424); |
| | | PURB(5814); YWHAG(7532); CDK6(1021); PDK4(5166); GPR85(54329); |
| | | HIPK2(28996); BRAF(673); LMBR1(64327); DLGAP2(9228); PTK2B(2185); |
| | | ESCO2(157570); FZD3(7976); RBPMS(11030); UNC5D(137970); BAG4 |
| | | (9530); TACC1(6887); SULF1(23213); TERF1(7013); CPNE3(8895); RIPK2 |
| | | (8767); WISP1(8840); ADRA1A(148); CLU(1191); SFRP1(6422); PAG |

Table 4-94

| | | |
|---|---|---|
| | | 1(55824); RBM12B(389677); TP53INP1(94241); ANGPT1(284); MTSS1(9788); |
| | | ANKRD15(23189); JAK2(3717); GDA(9615); OSTF1(26578); GCNT1 |
| | | (2650); VPS13A(23230); NTRK2(4915); MAK10(60560); DAPK1(1612); |
| | | C9orf47(286223); CKS2(1164); FCMD(2218); C9orf91(203197); PAPPA(5069); |
| | | GNAQ(2776); SEMA4D(10507); PTCH(5727); C9orf97(158427); C9orf5 |
| | | (23731); FBXW2(26190); TRAF1(7185); STRBP(55342); TSC1(7248); |
| | | WDR37(22884); CUGBP2(10659); TCF8(6935); CDC2(983); SGPL1(8879); |
| | | PTEN(5728); PCGF5(84333); KIF11(3832); ZRANB1(54764); DOCK1(1793); |
| | | LRRC27(80313); PIP5K2A(5305); CUL2(8453); PPP3CB(5532); DLG5 |
| | | (9231); SMNDC1(10285); DCLRE1A(9937); MKI67(4288); BNIP3(664); |
| | | STIM1(6786); IPO7(10527); NAV2(89797); HIPK3(10114); API5(8539); EXT2 |
| | | (2132); LRP5(4041); CCND1(595); BIRC3(330); BIRC2(329); CUL5(8065); |
| | | ATM(472); DIXDC1(85458); CBL(867); LOH11CR2A(4013); HRAS(3265); |
| | | EIF4G2(1982); LIN7C(55327); FBXO3(26273); SLC1A2(6506); TRAF6 |
| | | (7189); PGM2L1(283209); FZD4(8322); CTSC(1075); SESN3(143686); |
| | | BTG4(54766); PPP2R1B(5519); EVA1(10205); CCND2(894); RAD51AP1 |
| | | (10635); LTBR(4055); CNAP1(9918); TEGT(7009); MSRB3(253827); MDM2 |
| | | (4193); CRADD(8738); GAS2L3(283431); KNTC1(9735); ATP6V0A2(23545); |
| | | RAN(5901); TNFRSF1A(7132); OACT5(10162); KRAS(3845); FAIM2 |
| | | (23017); C12orf22(81566); CBX5(23468); OSBPL8(114882); PAWR(5074); |
| | | DUSP6(1848); BTG1(694); NTN4(59277); ANAPC7(51434); PPTC7(160760); |
| | | ANAPC5(51433); MPHOSPH9(10198); CDK2AP1(8099); CHFR(55743); |
| | | HSMPP8(54737); KIAA0774(23281); RP11-367C11.1(145173); BRCA2 |
| | | (675); RFP2(10206); SUGT1(10910); CUL4A(8451); LATS2(26524); USP12 |
| | | (219333); CSNK1A1L(122011); FOXO1A(2308); KCTD12(115207); POU4F1 |
| | | (5457); KIAA1333(55632); PNN(5411); TXNTDC(81542); ARID4A(5926); |
| | | MPP5(64398); CALM1(801); TRAF3(7187); TTC5(91875); C14orf126 |
| | | (112487); C14orf150(112840); CHES1(1112); BCL11B(64919); JAG2(3714); |
| | | SPRED1(161742); THBS1(7057); FGF7(2252); MAPK6(5597); CCNB2(9133); |
| | | KIF23(9493); AVEN(57099); MYEF2(50804); MYO5A(4644); ARPP-19 |
| | | (10776); PRTG(283659); CSNK1G1(53944); PDCD7(10081); TSPAN3(10099); |
| | | CCNF(899); DNASE1(1773); CYLD(1540); RBL2(5934); GNAO1(2775); |
| | | NFAT5(10725); GSPT1(2935); MAPK3(5595); DNAJA2(10294); FTS |
| | | (64400); CNOT1(23019); DYNC1LI2(1783); SMPD3(55512); EXOSC6(118460); |
| | | MAF(4094); HIC1(3090); PAFAH1B1(5048); CPD(1362); NF1(4763); |
| | | CDC6(990); CROP(51747); HLF(3131); PPM1D(8493); KPNA2(3838); MAP2K6 |
| | | (5608); SOX9(6662); BIRC5(332); UBE2G1(7326); DHX33(56919); |
| | | NALP1(22861); GAS7(8522); NCOR1(9611); FLJ36492(284047); BECN1 |
| | | (8678); SOCS3(9021); TBC1D16(125058); CSNK1D(1453); CEP192(55125); |
| | | RNMT(8731); CABLES1(91768); MAPRE2(10982); STATIP1(55250); SMAD4 |
| | | (4089); SOCS6(9306); YES1(7525); ZFP161(7541); ROCK1(6093); |
| | | ESCO1(114799); DSC2(1824); B4GALT6(9331); C18orf10(25941); PSTPIP2 |
| | | (9050); PIGN(23556); BCL2(596); PARD6G(84552); ZNF333(84449); UBA52 |
| | | (7311); PSMD8(5714); PPP2R1A(5518); NALP2(55655); MATK(4145); |
| | | ELAVL1(1994); ZNF561(93134); ZNF562(54811); CARD8(22900); MCM8 |
| | | (84515); PLCB1(23236); MAPRE1(22919); STK4(6789); C20orf35(55861); |
| | | RAB22A(57403); CSNK2A1(1457); PCNA(5111); PLAGL2(5326); RBL1 |
| | | (5933); CHD6(84181); UBE2V1(7335); ATP9A(10079); SYCP2(10388); |
| | | CABLES2(81928); DIDO1(11083); USP16(10600); SON(6651); CHAF1B |
| | | (8208); TMEM50B(757); BRWD1(54014); SNF1LK(150094); SMARCB1(6598); |
| | | KREMEN1(83999); APOL6(80830); TNRC6B(23112); EP300(2033); |
| | | BIK(638); PPARA(5465); MAPK1(5594); MN1(4330); PITPNB(23760); TOB2 |
| | | (10766); SMC1L2(27127); CELSR1(9620); USP9X(8239); PHF16(9767); |
| | | MAGEH1(28986); ZXDB(158586); YIPF6(286451); BIRC4(331); STAG2 |
| | | (10735); RP6-213H19.1(51765); CD40LG(959); FAM9C(171484); SCML2 |
| | | (10389); KLHL15(80311); RNF12(51132); RP11-130N24.1(340533); SEPT6 |
| | | (23157); CUL4B(8450); GPC4(2239); TMEM32(93380); CETN2(1069); MECP2 |
| | | (4204); |
| hsa-miR-548b | 2066; | THAP3(90326); SESN2(83667); LCK(3932); MACF1(23499); CAP1(10487); |
| | | MGC52498(348378); CACHD1(57685); SH3GLB1(51100); LRRC8C(84230); |
| | | SYCP1(6847); PEA15(8682); UHMK1(127933); BAT2D1(23215); PAPPA2 |
| | | (60676); ATP2B4(493); MFSD4(148808); PFKFB2(5208); TGFB2 |
| | | (7042); CCNL2(81669); E2F2(1870); KIAA0319L(79932); BMP8B(656); TESK2 |
| | | (10420); SLC16A4(9122); NRAS(4893); GLUL(2752); ENAH(55740); |
| | | C1orf198(84886); ERO1LB(56605); PRKCE(5581); TCF7L1(83439); KCN |

Table 4-95

| | | |
|---|---|---|
| | | IP3(30818); CNNM4(26504); BCL2L11(10018); ZAK(51776); DUSP19(142679); |
| | | CASP10(843); CD28(940); CARD12(58484); EIF2AK2(5610); BIN1 |
| | | (274); ARL5A(26225); NCKAP1(10787); STAT1(6772); FLJ22746(70843); |
| | | CUL3(8452); HDAC4(9759); PCAF(8850); LIMD1(8994); CCR9(10803); DTX3L |
| | | (151636); KLHDC6(166348); FXR1(8087); SENP2(59343); RASSF1 |
| | | (11186); SEMA3G(56920); PROK2(60675); KIAA1524(57650); STAG1(10274); |
| | | ATR(545); WHSC1(7468); HD(3064); ANAPC4(29945); KLF3(51274); |
| | | GUF1(60558); REST(5978); CCNG2(901); FGF5(2250); PHF17(79960); |
| | | LRPAP1(4043); PPP2R2C(5522); LDB2(9079); FLJ13197(79667); GNRHR |
| | | (2798); CASP6(839); SETD7(80854); SKP2(6502); ERBB2IP(55914); CCNB1 |
| | | (891); RGMB(285704); APC(324); RPL37(6167); BIRC1(4671); LOC153364 |
| | | (153364); SEMA6A(57556); PPP2CA(5515); SPRY4(81848); CAMK2A |
| | | (815); RNF130(55819); ZNF192(7745); OR2H1(26716); TRIM39(56658); |
| | | TCF19(6941); CSNK2B(1460); TCF21(6943); TBPL1(9519); HECA(51696); |
| | | SASH1(23328); C6orf145(221749); NEDD9(4739); ATXN1(6310); C6orf125 |
| | | (84300); IHPK3(117283); SUPT3H(8464); DST(667); SYNCRIP(10492); |
| | | FRK(2444); FBXO30(84085); LATS1(9113); TFB1M(51106); SEPT7(989); |
| | | STAG3(10734); RINT1(60561); CASP2(835); HOXA3(3200); CARD4 |
| | | (10392); KIAA0895(23366); ELMO1(9844); PBEF1(10135); WNT2(7472); |
| | | CREB3L2(64764); KIAA1456(57604); PTK2B(2185); BAG4(9530); HOOK3 |
| | | (84376); SULF1(23213); PI15(51050); RIPK2(8767); PTDSS1(9791); FZD6 |
| | | (8323); ZNF572(137209); WISP1(8840); XKR5(389610); PSD3(23362); |
| | | LZTS1(11178); TNFRS710A(8797); CLU(191); TPD52(7163); CCNE2(9134); |
| | | RAD21(5885); TNFRSF11B(4982); UNC13B(10497); NTRK2(4915); PTGS1 |
| | | (5742); ABL1(25); KIAA0649(9858); CDKN2A(1029); CDKN2B(1030); |
| | | AQP7(364); MGC21881(389741); SEMA4D(10507); DIRAS2(54769); C9orf5 |
| | | (23731); TSC1(7248); ANAPC2(29882); LRRTM3(347731); SGPL1(8879); |
| | | RAI17(57178); ZRANB1(54764); DOCK1(1793); ZWINT(11130); DLG5 |
| | | (9231); TLL2(7093); SMBP(56889); SLIT1(6585); C10orf46(143384); MKI67 |
| | | (4288); RPL27A(6157); EXT2(2132); INCENP(3619); VEGFB(7423); |
| | | FLJ10661(55199); PANX1(24145); BIRC2(329); CUL5(8065); ATM(472); |
| | | CBL(867); LOH11CR2A(4013); EI24(9538); CSTF3(1479); PHF21A(51317); |
| | | ZNF289(84364); PPP1CA(5499); SUV420H1(51111); FGF4(2249); RAB30 |
| | | (27314); FZD4(8322); PPP2R1B(5519); C11orf38(399967); PTPN6(5777); |
| | | FLJ20489(55652); ESPL1(9700); SP1(6667); MDM2(4193); CCT2(10576); |
| | | SOCS2(8835); CRADD(8738); C12orf23(90488); NOL1(4839); SLC2A3 |
| | | (6515); SENP1(29843); LALBA(3906); C12orf22(81566); CBX5(23468); DDIT3 |
| | | (1649); CTDSP2(10106); GNS(2799); PHLDA1(22822); DUSP6(1848); |
| | | FBXO21(23014); DIABLO(56616); FGF9(2254); BRCA2(675); NBEA(26960); |
| | | COG3(83548); ING1(3621); CDC16(8881); GJB2(2706); LATS2(26524); |
| | | NEK3(4752); NPAS3(64067); TXNDC(81542); CGRRF1(10668); DAD1 |
| | | (1603); TMEM30B(161291); TGFB3(7043); FLJ39531(400360); BUB1B |
| | | (701); BMF(90427); ELL3(80237); BNIP2(663); CSNK1G1(53944); DNASE1 |
| | | (1773); PRO0149(29035); PRRT2(112476); PAPD5(64282); HAS3(3038); |
| | | FLJ20186(54849); CREBBP(1387); FAM86A(196483); SMG1(23049); ERN2 |
| | | (10595); LOC440350(440350); CLAPIN1(57019); SMPD3(55512); TERF2 |
| | | (7014); FAM57A(79850); RPA1(6117); MYOCD(93649); NF1(4763); MAPT |
| | | (4137); NXPH3(11248); PPM1D(8493); PRKCA(5578); MAP2K6(5608); |
| | | KCNJ2(3759); GAS7(8522); FLJ36492(284047); TBC1D3C(414060); BECN1 |
| | | (8678); BRCA1(672); LOC641522(641522); CDC27(996); MPO(4353); |
| | | PCYT2(5833); RAB12(201475); TNFRSF11A(8792); SOCS6(9306); ESCO1 |
| | | (114799); CHAF1A(10036); RPS28(6234); ILF3(3609); UBA52(7311); |
| | | ZNF507(22847); APLP1(333); AXL(558); FOSB(2354); ZNF264(9422); APBA3 |
| | | (9546); RAB3D(9545); CARD8(22900); VISA(57506); PLCB1(23236); |
| | | PLCG1(5335); C20orf121(79183); CSTF1(1477); BIRC7(79444); RASSF2 |
| | | (9770); GGTL3(2686); KCNS1(3787); SPATA2(9825); DIDO1(11083); USP16 |
| | | (10600); SON(6651); PDXK(8566); BRWD1(54014); ITGB2(3689); KREMEN1 |
| | | (83999); NF2(4771); MCM5(4174); GGA1(26088); SEPT3(55964); BIK |
| | | (638); PPARA(5465); MAPK8IP2(23542); MAPK1(5594); CHEK2(11200); |
| | | HDAC6(10013); AGTR2(186); BIRC4(331); PPP2R3B(28227); EIF1AX |
| | | (1964); SMC1L1(8243); CUL4B(8450); MECP2(4204); XX-FW81657B9.4 |
| | | (8270); |
| hsa-miR-548d | 2067; | DFFB(1677); TNFRSF1B(7133); EPB41(2035); RBBP4(5928); YBX1(4904); |
| | | CDKN2C(1031); BTF3L4(91408); PRKAA2(5563); GIPC2(54810); SH3GLB1 |
| | | (51100); LRRC8C(84230); CDC14A(8556); SLC30A7(148867); BNI |

Table 4-96

PL(149428); CKS1B(1163); DAP3(7818); PEA15(8682); UHMK1(127933);
RALGPS2(55103); CAMSAP1L1(23271); MDM4(4194); IL19(29049); PFKFB2
(5208); TGFB2(7042); CDC2L2(985); CTNNBIP1(56998); DFFA(1676);
RCC2(55920); CLSPN(63967); POU3F1(5453); SLC35D1(23169); SERBP1
(26135); EVI5(7813); TMED5(50999); RSBN1(54665); NRAS(4893);
NOTCH2(4853); MCL1(4170); GLT25D2(23127); ZBTB41(360023); LPGAT1
(9926); TP53BP2(7159); KLF11(8462); FOSL2(2355); BIRC6(57448);
PRKCE(5581); HTLF(3344); GKN1(56287); HK2(3099); TCF7L1(83439);
FLJ13910(64795); INPP4A(3631); PDCL3(79031); BCL2L11(10018); RIF1
(55183); ZAK(51776); SGOL2(151246); CREB1(1385); COL4A3(1285); HRB
(3267); PSMD1(5707); SEPT2(4735); E2F6(1876); PPM1G(5496); GFPT1
(2673); TIA1(7072); TGFA(7039); ANAPC1(64682); IL1B(3553); SAP130
(79595); SESTD1(91404); BOLL(66037); ERBB4(2066); BARD1(580); HDAC4
(9759); CHL1(10752); VHL(7428); PCAF(8850); CRTAP(10491); TDGF1
(6997); C3orf14(57415); MITF(4286); QTRTD1(79691); ACPP(55); RAP2B
(5912); SMC4L1(10051); PIK3C(5290); KNG1(3827); OPA1(4976); TADA3L
(10474); ATP2B2(491); ANKRD28(23243); AXUD1(64651); PB1(55193);
WNT5A(7474); UBE1C(9039); PROK2(60675); VGLL3(389136); CGGBP1
(8545); DCBLD2(131566); KPNA1(3836); ZNF148(7707); STAG1
(10274); PIK3CB(5291); ATR(545); SIAH2(6478); AADACL1(57552); TBL1XR1
(79718); PPP1R2(5504); CPEB2(132864); HCAP-G(64151); RBPSUH
(3516); ENAM(10117); IL8(3576); CXCL6(6372); EREG(2069); SEPT11(55752);
FGF5(2250); TLR2(7097); GAK(2580); CCN1(10983); KLHL8(57563);
SNCA(6622); LEF1(51176); CASP6(839); MAD2L1(4085); SETD7(80854);
NEK1(4750); CASP3(836); SUB1(10923); CARD6(84674); R7BP(401190);
FLJ13611(80006); ERBB2IP(55914); RAD17(5884); APC(324); CSNK1G3
(1456); TCF7(6932); MATR3(9782); GRPEL2(134266); SGCD(6444);
SEMA5A(9037); PRLR(5618); RICTOR(253260); PRKAA1(5562); BIRC1
(4671); ATG12(9140); SEMA6A(57556); IRF1(3659); CSNK1A1(1452); C5orf3
(10827); DUSP1(1843); E2F3(1871); CSNK2B(1460); VEGF(7422); CDC5L
(988); TTK(7272); FOXO3A(2309); TNFAIP3(7128); HECA(51696); SASH1
(23328); QKI(9444); SSR1(6745); NEDD9(4739); C6orf62(81688); IHPK3
(117283); SUPT3H(8464); DSCR1L1(10231); TNFRSF21(27242); DST
(667); EEF1A1(1915); BACH2(60468); CCNC(892); STX7(8417); SGK
(6446); HBS1L(10767); MAP3K5(4217); PLAGL1(5325); LATS1(9113); SOD2
(6648); AHR(196); HDAC9(9734); EGFR(1956); ZNF273(10793); LOC441257
(441257); PFTK1(5218); RINT1(60561); PRKAR2B(5577); CASP2
(835); CARD4(10392); INHBA(3624); IGFBP3(3486); YWHAG(7532); CDK6
(1021); PERQ1(64599); ZC3HAV1(56829); HIPK2(28996); CDK5(1020);
MLL3(58508); TNKS(8658); TNFRS710C(8794); FZD3(7976); UNC5D(137970);
TACC1(6867); SULF1(23213); TERF1(7013); E2F5(1875); FZD6(8323);
TNFRSF10B(8795); TNFRSF10D(8793); TNFRSF10A(8797); WHSC1L1
(54904); SFRP1(6422); VCPIP1(80124); ZNF704(619279); PAG1(55824);
MMP16(4325); TMEM64(169200); TP53INP1(94241); ZNF706(51123);
RAD21(5885); EXT1(2131); MTSS1(9788); CDC37L1(55664); TGFBR1
(7046); SLC44A1(23446); C9orf28(89853); ABL1(25); TAF1L(138474); SMU1
(55234); RNF38(152006); SLC28A3(64078); EPB41L4B(54566); ROD1
(9991); TNFSF8(944); FNBP1(23048); RAPGEF1(2889); TSC1(7248); VAV2
(7410); BRD3(8019); ZMYND11(10771); C10orf9(219771); SGPL1(8879);
BMPR1A(657); PTEN(5728); FAS(355); PCGF5(84333); FRAT1(10023);
ENTPD7(57089); BTRC(8945); PDCD11(22984); CSPG6(9126); TCF7L2
(6934); BAG3(9531); LARP5(23185); PARD3(56288); ZNF248(57209); CXCL12
(6387); ANUBL1(93550); CCDC6(8030); COX15(1355); FGF8(2253);
SMNDC1(10285); DCLRE1A(9937); C10orf46(143384); TIAL1(7073);
WEE1(7465); MLSTD2(84188); NAV2(89797); HIPK3(10114); RAG1(5896);
API5(8539); DPF2(5977); FBXL11(22992); CCND1(595); CTTN(2017);
BIRC3(330); BIRC2(329); CUL5(8065); ATM(472); MLL(4297); EI24(9538);
NUP98(4928); EIF4G2(1982); HRASLS3(11145); FZD4(8322); JOSD3(79101);
CASP1(834); PPP2R1B(5519); DDX6(1656); BCL9L(283149); CCND2
(894); CDKN1B(1027); ETNK1(55500); METTL7A(25840); CDK2(1017);
PA2G4(5036); MDM2(4193); CRADD(8738); APAF1(317); SCYL2(55681);
GAS2L3(283431); RAN(5901); PARP11(57097); BCAT1(586); KRAS(3845);
IPO8(10526); SLC2A13(114134); YAF2(10138); HDAC7A(51564); C12orf22
(81566); PAWR(5074); PCTK2(5128); DIP13B(55198); PPP1CC(5501);
MPHOSPH9(10198); CDK2AP1(8099); HSMPP8(54737); ATP8A2(51

Table 4-97

| | | |
|---|---|---|
| | | 761); WASF3(10810); BRCA2(675); AKAP11(11215); RB1(5925); RFP2(10206); |
| | | CUL4A(8451); XPO4(64328); LATS2(26524); KATNAL1(84056); DCAMKL1 |
| | | (9201); FOXO1A(2308); KLF12(11278); PNN(5411); SOCS4(122809); |
| | | PELI2(57161); STRN3(29966); NFKBIA(4792); WDHD1(11169); CHES1 |
| | | (1112); BCL11B(64919); SETD3(84193); BAG5(9529); AKT1(207); SPRED1 |
| | | (161742); BUB1B(701); MAPK6(5597); CCNB2(9133); IGF1R(3480); |
| | | AVEN(57099); BMF(90427); MYEF2(50804); COPS2(9318); BNIP2(663); |
| | | CSNK1G1(53944); PDCD7(10081); C15orf38(348110); CYLD(1540); RBL2 |
| | | (5934); RSPRY1(89970); PARD6A(50855); CREBBP(1387); GSPT1(2935); |
| | | MAPK3(5595); SIAH1(6477); FTS(64400); CMTM4(146223); EXOSC6 |
| | | (118460); ZCCHC14(23174); PAFAH1B1(5048); NLK(51701); NF1(4763); |
| | | CDK5R1(8851); CCDC16(91603); TADA2L(6871); EIF1(10209); FZD2(2535); |
| | | CROP(51747); MSI2(124540); PPM1D(8493); TLK2(11011); PRKCA |
| | | (5578); BIRC5(332); TP53(7157); GAS7(8522); TIAF1(9220); NUFTP2(57532); |
| | | BECN1(8678); ERN1(2081); DDX5(1655); SMURF2(64750); HELZ(9931); |
| | | SOCS3(9021); TWSG1(57045); CABLES1(91768); C18orf16(147429); |
| | | C18orf25(147339); DCC(1630); C18orf54(162681); DOK6(220164); SS18 |
| | | (6760); PARD6G(84552); FZR1(51343); UHRF1(29128); OR7D2(162998); |
| | | UBA52(7311); CRTC1(23373); CCNE1(898); SAMD4B(55095); MAP3K10 |
| | | (4294); APOE(348); PPP2R1A(5518); LOC390980(390980); MKNK2(2872); |
| | | PLEKHJ1(55111); AES(166); SLC25A23(79085); TNFSF7(970); INSR(3643); |
| | | ZFP30(22835); KLK10(5655); ZNF468(90333); PLCB1(23236); MAPRE1 |
| | | (22919); CBFA2T2(9139); CEP250(11190); STK4(6789); CSE1L(1434); |
| | | STX16(8675); CSNK2A1(1457); BCL2L1(598); E2F1(1869); RBM12(10137); |
| | | UBE2V1(7335); SYCP2(10388); CABLES2(81928); DIDO1(11083); GM632 |
| | | (57473); USP16(10600); SLC5A3(6526); DYRK1A(1859); BTG3(10950); |
| | | BRWD1(54014); ZNF295(49854); SNF1LK(150094); BCL2L13(23786); |
| | | CRKL(1399); TNRC6B(23112); BIK(638); PPARA(5465); YPEL1(29799); |
| | | MAPK1(5594); PPM1F(9647); TPST2(8459); DMC1(11144); SMC1L2(27127); |
| | | CELSR1(9620); WWC3(55841); ZFX(7548); PRRG1(5638); XK(7504); |
| | | USP9X(8239); HDAC6(10013); MLLT7(4303); BIRC4(331); STAG2(10735); |
| | | RP6-213H19.1(51765); RPL10(6134); PPP2R3B(28227); TRAPPC2(6399); |
| | | PIGA(5277); EIF1AX(1964); KLHL15(80311); DMD(1756); SMC1L1 |
| | | (8243); WNK3(65267); RNF12(51132); SEPT6(23157); TMEM32(93380); IDS |
| | | (3423); MECP2(4204); |
| has-miR-550 | 2068; 2126; | ZBTB8(127557); MACF1(23499); RAP1A(5906); BCL9(607); DDR2(4921); |
| | 2127; | IPO9(55705); IL24(11009); DISC1(27185); FAF1(11124); DEDD(9191); CCT7 |
| | | (10574); ZAK(51776); FLJ38973(205327); TGFA(7039); APPL(26060); |
| | | PCNP(57092); SENTP2(59343); TP73L(8626); THRB(7068); CD47(961); KIAA2018 |
| | | (205717); FLJ35725(152992); PRKG2(5593); F2R(2149); PRLR |
| | | (5618); P18SRP(285672); SMA5(11042); MCC(4163); PPP2CA(5515); NME5 |
| | | (8382); TTK(7272); AIM1(202); HECA(51696); C6orf120(387263); MDC1 |
| | | (9656); SYNCR1P(10492); SEPT7(989); HBP1(26959); UNC5D(137970); FKSG2 |
| | | (59347); BAG4(9530); SULF1(23213); ADRA1A(148); TP53INP1(94241); |
| | | NCALD(83988); CDC37L1(55664); RECK(8434); DAPK1(1612); BAG1 |
| | | (573); CUGBP2(10659); C10orf9(219771); SIRT1(23411); EIF4EBP2 |
| | | (1979); NUDT5(11164); ZWINT(11130); PHCA(55331); BIRC3(330); CHEK1 |
| | | (1111); IGF2(3481); RIC3(79608); DYRK2(8445); MDM2(4193); GAS2L3 |
| | | (283431); BCAT1(586); RFP2(10206); MNAT1(4331); TRAF3(7187); AVEN |
| | | (57099); CYLD(1540); RBL2(5934); GNG13(51764); C16orf28(65259); GSPT1 |
| | | (2935); NF1(4763); LIG3(3980); CNP(1267); NALP1(22861); MYH10 |
| | | (4628); LIMD2(80774); CABLES1(91768); C18orf8(29919); SYT4(6860); CSNK1G2 |
| | | (1455); ATF5(22809); PLCB1(23236); SERINC3(10955); USP16 |
| | | (10600); BIK(638); MAPK1(5594); STAG2(10735); SEPT6(23157); |
| has-miR-551b | 2069; 2122; | C1orf93(127281); C1orf144(26099); LDLRAP1(26119); TMEM56(148534); |
| | 2128; | C1orf119(56900); ADAMTSL4(54507); ATP8B2(57198); SDHC (6391); RASSF5 |
| | | (83593); KIAA1804(84451); TNFRSF18(8784); RERE(473); FAM46B |
| | | (115572); TTMB(399474); CSMD2(114784); KIAA0319L(79932); HEYL |
| | | (26508); NOTCH2(4853); PDE4DIP(9659); CD5L(922); TNNI1(7135); BIRC6 |
| | | (57448); EPAS1(2034); CCT7(10574); KCNIP3(30818); MRPL30(51263); |
| | | C2orf26(65124); PNKD(25953); E2F6(1876); DNMT3A(1788); SFXN5 |
| | | (94097); FLJ20297(55627); ERBB4(2066); PRKAG3(53632); ZNF35(7584); |
| | | HEMK1(51409); ABHD6(57406); ATXN7(6314); TADA3L(10474); USP4 |
| | | (7375); ARHGEF3(50650); MAGI1(9223); C3orf17(25871); RAB6B(51560); |
| | | LOC348840(348840); HD(3064); STK32B(55351); SORCS2(57537); GA |

Table 4-98

| | | |
|---|---|---|
| | | LNT17(442117); ZFYVE28(57732); FLJ90013(202018); DDIT4L(115265); |
| | | ANKRD50(57182); FLJ38482(201931); MFAP3L(9848); CCNB1(891); PCDHB9 |
| | | (56127); PCDHB14(56122); LTC4S(4056); SQSTM1(8878); ARTS- |
| | | 1(51752); MRS2L(57380); ANKS1A(23294); PARC(23113); C6orf71(389434); |
| | | GMDS(2762); GPX6(257202); TRIM31(11074); IER3(8870); TAPBP(6892); |
| | | CUL7(9820); C6orf168(84553); BCLAF1(9774); FLJ44955(401278); |
| | | PDCD2(5134); KIAA0415(9907); ZNF498(221785); C7orf19(80228); CASP2 |
| | | (835); HOXA7(3204); YWHAG(7532); KBTBD11(9920); BAG4(9530); PTP4A3 |
| | | (11156); ZNF707(286075); MAPK15(225689); KIAA1875(340390); |
| | | HSF1(3297); GPT(2875); XKR5(389610); TNFRSF10B(8795); KIF13B(23303); |
| | | MYST3(7994); PAG1(55824); ST3GAL1(6482); PTK2(5747); JRK(8629); |
| | | EPPK1(83481); SCRT1(83482); ADAMTSL1(92949); ASAH3L(340485); |
| | | NTRK2(4915); DAPK1(1612); PHF2(5253); CRB2(286204); C9orf58(83543); |
| | | PTGDS(5730); GRIN1(2902); NDOR1(27158); GLIS3(169792); BNC2 |
| | | (54796); TMEM2(23670); DENND1A(57706); ST6GALNAC6(30815); FLJ20433 |
| | | (54932); NCOA4(8031); LOC439985(439985); C10orf116(10974); |
| | | NEURL(9148); DOCK1(1793); CCDC3(83643); NEUROG3(50674); ANXA11 |
| | | (311); OBFC1(79991); C10orf46(143384); MGC45840(283229); CD81 |
| | | (975); HIPK3(10114); PELI3(246330); RAD9A(5883); HTR3B(9177); FAM55B |
| | | (120406); LOH11CR2A(4013); CHID1(66005); TAF10(6881); FBXO3(26273); |
| | | ZNF289(84364); LTBP3(4054); PPP2R1B(5519); BCL9L(283149); |
| | | NDUFA9(4704); LRRC23(10233); DDX11(1663); SCN8A(6334); CHST11 |
| | | (50515); PGAM5(192111); RAD52(5893); CACNA2D4(93589); NRIP2(83714); |
| | | GRIN2B(2904); TIMELESS(8914); PCTK2(5128); BRCA2(675); LMO7; |
| | | (4008); ITGBL1(9358); SLC7A1(6541); DKFZp451A211(400169); ADPRHL1 |
| | | (113622); MMP14(4323); FLJ38964(161253); C14orf21(161424); FRMD6 |
| | | (122786); DLG7(9787); CDC42BPB(9578); PPP1R13B(23368); LOC400968 |
| | | (400968); SCG3(29106); CCNB2(9133); FAM81A(145773); NEO1 |
| | | (4756); SH3PX3(257364); TMED3(23423); IGF1R(3480); TLE3(7090); SEMA7A |
| | | (8482); SELS(55829); NUBP2(10101); E4F1(1877); MMP25(64386); |
| | | IL4R(3566); ATP2A1(487); PRRT2(112476); NUP93(9688); FOXL1(2300); |
| | | ZNF276(92822); MGC21830(283870); ADCY9(115); C16orf5(29965); C16orf51 |
| | | (25880); GSPT1(2935); BOLA2(552900); STX1B2(112755); LOC283849 |
| | | (283849); ATP6V0D1(9114); FBXO31(79791); RPA1(6117); PAFAH1B1 |
| | | (5048); NDEL1(81565); COX10(1352); MAPK7(5598); ZNHITS(9326); |
| | | THRA(7067); RAPGEFL1(51195); RSAD1(55316); ACE(1636); FAM100B |
| | | (283991); CBX2(84733); SMYD4(114826); RDM1(201299); BECN1(8678); |
| | | SP6(80320); HOXB9(3219); SOCS3(9021); CANT1(124583); TBC1D16(125058); |
| | | PCYT2(5833); RFNG(5986); KIAA0427(9811); DOK6(220164); C18orf2 |
| | | (56651); LPIN2(9663); PARD6G(84552); SPPL2B(56928); TNFAIP8L1 |
| | | (126282); F2RL3(9002); KIAA0892(23383); MGC32020(91442); PSMD8 |
| | | (5714); ZFP36(7538); APOE(348); RCN3(57333); KLK3(354); ZNF581(51545); |
| | | REXO1(57455); SLC39A3(29985); PLAC2(257000); MGC34725(284427); |
| | | ZNF562(54811); LPHN1(22859); RASGRP4(115727); ERCC2(2068); |
| | | CABP5(56344); KLK4(9622); PTPRH(5794); IL11(3589); OTOR(56914); FLJ44790 |
| | | (400850); ZHX3(23051); C20orf175(140876); AURKA(6790); LAMA5 |
| | | (3911); DIDO1(11083); OLIG1(116448); SLC5A3(6526); MX1(4599); |
| | | CRYAA(1409); PCBP3(54039); PRMT2(3275); RIPK4(54101); C21orf29(54084); |
| | | C21orf69(84537); LSS(4047); DGCR8(54487); UPB1(51733); SH3BP1 |
| | | (23616); CECR6(27439); TOB2(10766); IL3RA(3563); GLRA2(2742); FLJ21687 |
| | | (79917); RP1-112K5.2(90121); MAP3K15(389840); ARHGEF9(23229); |
| | | PJA1(64219); RP11-130N24.1(340533); FAM70A(55026); SUHW3 |
| | | (55609); |
| hsa-miR-552 | 2070; | PRDM16(63976); THAP3(90326); TNFRSF1B(7133); C1orf151(440574); |
| | | NUDC(10726); KHDRBS1(10657); NFYC(4802); KIAA0467(23334); BTF3L4 |
| | | (91408); ZYG11B(79699); ECAT11(54596); SH3GLB1(51100); CDC14A |
| | | (8556); GPSM2(29899); PROK1(84432); VANGL1(81839); TTF2(8458); |
| | | BCL9(607); CKS1B(1163); HCN3(57657); FDPS(2224); PEA15(8682); SLAMF7 |
| | | (57823); C1orf76(148753); IPO9(55705); TRAF5(7188); TGFB2(7042); |
| | | RHOU(58480); C1orf124(83932); EDARADD(128178); CIAS1(114548); |
| | | CCNL2(81669); C1orf86(199990); TNFRSF14(8764); TNFRSF9(3604); CTNNBIP1 |
| | | (56998); DFFA(1676); FRAP1(2475); C1orf126(200197); FBXO42 |
| | | (54455); RCC2(55920); AFAR3(246181); IL28RA(163702); MECR(51102); |
| | | HPCAL4(51440); MUTYH(4595); TESK2(10420); CYP4A11(1579); SPATA6 |
| | | (54558); AGBL4(84871); FAF1(11124); DAB1(1600); RBMXL1(4941 |

Table 4-99

15); NOTCH2(4853); PYGO2(90780); ISG20L2(81875); FCRL5(83416); CD84
(8832); ANKRD45(339416); GLUL(2752); ASPM(259266); RBBP5(5929);
RAB7L1(8934); IL10(3586); ENAH(55740); TFB2M(64216); RNF144
(9781); FLJ40869(348654); MAPRE3(22924); BIRC6(57448); CCDC75(253635);
GALM(130589); MSH2(4436); HK2(3099); TCF7L1(83439); FLJ20758
(55037); BRRN1(23397); C2orf15(150590); PROC(5624); RIF1(55183);
ZAK(51776); PMS1(5378); COQ10B(80219); CFLAR(8837); CASP10(843);
CYP20A1(57404); CD28(940); ALS2CR19(117583); CREB1(1385); ATG4B
(23192); ING5(84289); OACT2(129642); CGREF1(10669); CARD12(58484);
BCL11A(53335); DKFZp686K16132(388957); TGFA(7039); FLJ90024
(129303); REEP1(65055); EDAR(10913); RGPD5(84220); FLJ40629(150468);
IL1B(3553); FLJ16008(339761); LIMS2(55679); IFIH1(64135); FLJ13984
(79828); CUL3(8452); EDEM1(9695); OGG1(4968); FANCD2(2177);
SLC6A6(6533); PDCD6IP(10015); CTDSPL(10217); DLEC1(9940); MYD88
(4615); CTNNB1(1499); LIMD1(8994); ARGFX(503582); ACPP(55); FXR1
(8087); ST6GAL1(6480); RAF1(5894); IQSEC1(9922); MRPS25(64432);
THRB(7068); AXUD1(64651); KIAA1143(57456); SLC6A20(54716); NICN1
(84276); IHPK1(9807); RASSF1(11186); TMEM110(375346); CHDH
(55349); STAG1(10274); MGC40579(256356); TNFSF10(8743); EHHADH
(1962); LRRC15(131578); PCGF3(10336); WHSC1(7468); HD(3064); SORCS2
(57537); CD38(952); KLB(152831); DKFZP564O0823(25849); CCNG2
(901); SPP1(6696); PCDH10(57575); WDR17(116966); WWC2(80014); SLC25A4
(291); MXD4(10608); LRPAP1(4043); GRSF1(2926); PPEF2(5470);
FLJ13614(84142); UNC5C(8633); MAP2K1IP1(8649); DNAJB14(79982);
CASP6(839); TIFA(92610); SFRP2(6423); NEK1(4750); SPATA4(132851);
NIPBL(25836); GTF2H2(2966); MAP1B(4131); F2R(2149); APC(324);
DCP2(167227); LOC90624(90624); TCF7(6932); UBE2B(7320); ARHGAP26
(23092); NDST1(3340); GALNT10(55568); ERGIC1(57222); BNIP1(662);
ZNF346(23567); SLC34A1(6569); SEMA5A(9037); LOC134145(134145);
DAP(1611); MYO10(4651); BIRC1(4671); DHFR(1719); ATG12(9140);
DTWD2(285605); IRF1(3659); DPYSL3(1809); PDE6A(5145); CAMK2A
(815); FBXW11(23291); GRM6(2916); RIPK1(8737); TCF19(6941); CSNK2B
(1460); C6orf153(88745); PTK7(5754); IL17(3605); FBXO9(26268); PRDM1
(639); QRSL1(55278); KIAA1919(91749); SLC35D3(340146); PHACTR2
(9749); SASH1(23328); MDC1(9656); BAK1(578); NUDT3(11165); TEAD3
(7005); USP49(25862); CCND3(896); SUPT3H(8464); DST(667); GUSBL2
(375513); UBE2J1(51465); SGK(6446); BCLAF1(9774); PLAGL1(5325);
LATS1(9113); AGPAT4(56895); PDCD2(5134); TMEM106B(54664); AHR
(196); STK17A(9263); DKFZp761I2123(83637); ADCY1(107); ZNFN1A1
(10320); EGFR(1956); VKORC1L1(154807); HSPB1(3315); NBLA04196
(64921); ZNF655(79027); BCAP29(55973); MDFIC(29969); KIAA0265(23008);
LOC155060(155060); MGC9712(202915); TRIAD3(54476); CYCS(54205);
CARD4(10392); TMED4(222068); COBL(23242); FLJ44060(346288);
PMS2L3(5387); CDK6(1021); SAMD9L(219285); GATS(352954); ALKBH4
(54784); POLR2J3(548644); POLR2J2(246721); SLC35B4(84912); PTN
(5764); CREB3L2(64764); HIPK2(28996); ABCF2(10061); PTPRN2(5799);
CLN8(2055); PDLIM2(64236); BNIP3L(665); RBPMS(11030); NRG1
(3084); UNC5D(137970); SPFH2(11160); BAG4(9530); DDHD2(23259); TACC1
(6867); PTDSS1(9791); FZD6(8323); KIAA0143(23167); GML(2765);
ZNF623(9831); ZNF7(7553); XKR5(389610); KCTD9(54793); TRIM35(23087);
KIF13B(23303); FGFR1(2260); ZNF704(619279); CCNE2(9134); TP53INP1
(94241); TSPYL5(85453); HRSP12(10247); TRPS1(7227); RAD21
(5885); ST3GAL1(6482); JAK2(3717); MTAP(4507); UNC13B(10497); PIP5K1B
(8395); GCNT1(2650); DAPK1(1612); C9orf47(286223); SYK(6850);
STX17(55014); INVS(27130); PALM2(114299); SLC31A1(1317); DAB2IP
(153090); C9orf28(89853); FAM73B(84895); ABL1(25); C9orf58(83543); TRAF2
(7186); BNC2(54796); TAF1L(138474); ZBTB5(9925); TRPM3(80036);
C9orf40(55071); TLE1(7088); GAS1(2619); ZNF510(22869); TRIM14(9830);
ANKS6(203286); TNFSF15(9966); PSMD5(5711); TRAF1(7185); CIZ1
(25792); ASB6(140459); TSC1(7248); CAMK1D(57118); C10orf58(84293);
NOLC1(9221); TCF7L2(6934); NANOS1(340719); BAG3(9531); PAPD1
(55149); PARD3(56288); LRRC18(474354); C10orf72(196740); AP3M1(26985);
C10orf95(79946); C10orf46(143384); MKI67(4288); BNIP3(664); IFITM1
(8519); TEAD1(7003); HIPK3(10114); API5(8539); HSD17B12(51144);
EXT2(2132); CDC42EP2(10435); DPF2(5977); SSSCA1(10534); SIPA1

Table 4-100

(6494); PELI3(246330); RBM14(10432); LRP5(4041); CCND1(595); PHCA
(55331); BIRC3(330); BIRC2(329); ATM(472); SNF1LK2(23235); TAGLN
(6876); CD3E(916); BET1L(51272); APBB1(322); RIC3(79608); SBF2(81846);
C11orf55(399879); SLC15A3(51296); CDCA5(113130); AYP1(84153); CLCF1
(23529); PPP1CA(5499); PGM2L1(283209); GDPD5(81544); PAK1
(5058); SLC36A4(120103); CASP4(837); PPP2R1B(5519); ASAM(79827); CCND2
(894); NTF3(4908); CNAP1(9918); USP5(8078); PTPN6(5777); ATF7IP
(55729); ESPL1(9700); DKFZP586D0919(25895); SLC35E3(55508); CCT2
(10576); SOCS2(8835); P2RX7(5027); FLJ38663(91574); TMEM132B
(114795); FZD10(11211); NOC4L(79050); NOL1(4839); C1RL(51279); AICDA
(57379); MGC24039(160518); FAIM2(23017); GALNT6(11226); K6HF
(9119); CDK4(1019); CTDSP2(10106); C12orf31(84298); CPM(1368); TMCC3
(57458); FBXO21(23014); RHOF(54509); VPS37B(79720); SBNO1(55206);
CHFR(55743); ATP8A2(51761); BRCA2(675); AKAP11(11215); LOC440138
(440138); FARP1(10160); ATP11A(23250); CUL4A(8451); FOXO1A
(2308); TPT1(7178); DACH1(1602); FLJ10154(55082); DKFZp451A211(400169);
PAPLN(89932); JDP2(122953); VASH1(22846); TRAF3(7187); C14orf153
(84334); SUPT16H(11198); ACIN1(22985); DHRS7(51635); TRMT5
(57570); LTBP2(4053); SPTLC2(9517); CHES1(1112); C14orf103(55102);
AKT1(207); JAG2(3714); CEP27(55142); PLDN(26258); SEMA6D(80031);
SNX1(6642); PTPLAD1(51495); SMAD3(4088); KIF23(9493); PML(5371);
CSK(1445); DNAJA4(55466); CHRNA5(1138); FLJ22795(80154); SCAND2
(54581); RCCD1(91433); ST8SIA2(8128); SLC12A6(9990); BMF
(90427); TTBK2(146057); TNFAIP8L3(388121); RAB27A(5873); PDCD7(10081);
BRUNOL6(60677); C15orf38(348110); RAB40C(57799); TBL3(10607);
TRAF7(84231); CCNF(899); DNASE1(1773); UBN1(29855); RUNDC2A
(84127); UBPH(56061); TNT(162083); SPN(6693); PRRT2(112476); ITGAX
(3687); CYLD(1540); NUP93(9688); ELMO3(79767); MARVELD3(91862);
CDT1(81620); C16orf28(65259); NME3(4832); GSPT1(2935); XYLT1
(64131); ERN2(10595); P8(26471); FLJ32130(146540); CIAPIN1(57019);
TK2(7084); TUSC5(286753); MGC88387(400569); ALOX15B(247); MED9
(55090); LLGL1(3996); EPN2(22905); WSB1(26118); NLK(51701); TMEM97
(27346); SARM1(23098); GOSR1(9527); NF1(4763); RAB11FIP4(84440);
LIG3(3980); RUNDC1(146923); TMEM106A(113277); ASB16(92591);
DBF4B(80174); SP2(6668); PNPO(55163); CALCOCO2(10241); TOM1L1
(10040); AKAP1(8165); PRR11(55771); RPS6KB1(6198); METTL2A(339175);
WDR68(10238); SSTR2(6752); TTYH2(94015); KIF19(124602); TMEM104
(54868); BIRC5(332); C17orf27(57674); BAIAP2(10458); PITPNA
(5306); SMYD4(114826); TSR1(55720); MNT(4335); GGT6(124975); TP53
(7157); GAS7(8522); TOM1L2(146691); ULK2(9706); C17orf63(55731); NUFIP2
(57532); FLJ31952(146857); PCGF2(7703); FBXL20(84961); SC65
(10609); BRCA1(672); GJA7(10052); C17orf73(55018); USH1G(124590);
RECQL5(9400); MXRA7(439921); DNAH17(8632); USP36(57602); TIMP2
(7077); TBC1D16(125058); NPTX1(4884); FLJ90757(440465); USP14(9097);
C18orf1(753); RNMT(8731); MAPRE2(10982); SMAD4(4089); DCC
(1630); SERPINB2(5055); ZNF24(7572); SMAD2(4087); MBD2(8932); BCL2
(596); FVT1(2531); TXNL4A(10907); PARD6G(84552); CDC34(997); CSNK1G2
(1455); FZR1(51343); HMG20B(10362); EBI3(10148); TNFAIP8L1
(126282); UHRF1(29128); ILF3(3609); LOC284434(284434); UBA52(7311);
ZNF430(80264); ZNF546(339327); RAB4B(53916); AXL(558); SPHK2
(56848); ATF5(22809); SPIB(6689); KLK3(354); NALP2(55655); ZNF211
(10520); POLR2E(5434); AES(166); FUT6(2528); INSR(3643); ZN7490(57474);
PRDX2(7001); BRD4(23476); TRA16(126382); ZNF565(147929); KIAA1559
(57677); FBXO27(126433); OPA3(80207); CARD8(22900); ZFPL
(162967); ZNF160(90338); ZNF667(63934); VISA(57506); HM13(81502); TTLL9
(164395); ASXL1(171023); C20orf24(55969); SRC(6714); TOP1(7150);
MYBL2(4605); C20orf121(79183); STK4(6789); ZSWIM1(90204); CD40
(958); PARD6B(84612); GTPBP5(26164); SLC4A11(83959); COMMD7(149951);
GGTL3(2686); ZHX3(23051); ELMO2(63916); KCNB1(3745); UBE2V1
(7335); NFATC2(4773); BMP7(655); CTCFL(140690); SYCP2(10388);
DIDO1(11083); HUNK(30811); IFNAR1(3454); SON(6651); SIM2(6493);
PKNOX1(5316); PDXK(8566); APP(351); GART(2618); BRWD1(54014); BCL2L13
(23786); DGCR8(54487); ZNF74(7625); P2RXL1(9127); LOC284861
(284861); OSBP2(23762); MCM5(4174); SEPT3(55964); PPARA(5465);
ADM2(79924); hCAP-H2(29781); FLJ21125(79680); MGC16703(113691)

Table 4-101

| | | |
|---|---|---|
| | | ; MAPK1(5594); FLJ31568(150244); RP5-1119A7.4(80020); FLJ90680(400926); |
| | | CARD10(29775); C22orf5(25829); DMC1(11144); NPTXR(23467); PDGFB |
| | | (5155); DNAJB7(150353); NFAM1(150372); C22orf9(23313); WNT7B |
| | | (7477); LOC440836(440836); OATL1(4943); HDAC6(10013); TAF1(6872); |
| | | OGT(8473); RP6-213H19.1(51765); ARSD(414); CXorf20(139105); PCYT1B |
| | | (9468); SMC1L1(8243); LAMP2(3920); ARHGEF6(9459); CETN2(1069); |
| | | PDZD4(57595); HCFC1(3054); RAB39B(116442); PRKY(5616); |
| hsa-miR-553 | 2071; | H6PD(9563); TNFRSF8(943); PDPN(10630); LDLRAD2(401944); CDC42 |
| | | (998); ATPIF1(93974); ZYG11B(79699); ANGPTL3(27329); SH3GLB1(51100); |
| | | C1orf82(79871); GPR88(54112); RBM8A(9939); IL6R(3570); CD1D |
| | | (912); SLAMF8(56833); FASLG(356); C1orf21(81563); NAV1(89796); ELF3 |
| | | (1999); C1orf95(375057); DISC1(27185); FMN2(56776); MTHFR(4524); HIVEP3 |
| | | (59269); SSBP3(23648); GFI1(2672); RP5-998N21.6(440686); TPM3 |
| | | (7170); SHC1(6464); FCRL1(115350); TADA1L(117143); ZNF648(127665); |
| | | C1orf116(79098); CD34(947); SLC30A1(7779); EPRS(2058); TAF5L(27097); |
| | | ZNF496(84838); FLJ41046(400940); RNF144(9781); SELI(85465); |
| | | DPYSL5(56896); LBH(81606); BIRC6(57448); SPTBN1(6711); FLJ13910 |
| | | (64795); TMEM87B(84910); ZC3H6(376940); ACTR3(10096); INHBB(3625); |
| | | RAB6C(84084); UBXD2(23190); TANK(10010); MAP1D(254042); HNRPA3 |
| | | (220988); FLJ20160(54842); FLJ32063(150538); CASP10(843); CREB1 |
| | | (1385); HRB(3267); ASB1(51665); FAM49A(81553); WDR35(57539); |
| | | SLC5A6(8884); BCL11A(53335); MRPS5(64969); RNF149(284996); LOC130074 |
| | | (130074); MCM6(4175); RPRM(56475); STK39(27347); KIAA1715 |
| | | (80856); SLC40A1(30061); RAPH1(65059); WDFY1(57590); GRM7(2917); |
| | | TMEM43(79188); NR2C2(7182); CAPN7(23473); EAF1(85403); ITGA9(3680); |
| | | OXSR1(9943); WDR48(57599); DOCK3(1795); FAIM(55179); SPSB4 |
| | | (92369); LPP(4026); FAM79B(285386); RPL32(6161); WNT7A(7476); USP4 |
| | | (7375); AER61(285203); CD47(961); TMEM39A(55254); CD80(941); RAB43 |
| | | (339122); AMOTL2(51421); STAG1(10274); NMNAT3(349565); ZIC4 |
| | | (84107); C3orf57(165679); DGKG(1608); CENTB2(23527); MGC4618(84286); |
| | | TBC1D14(57533); LOC285429(285429); REST(5978); DKFZP564O0823 |
| | | (25849); ARFIP1(27236); TLR2(7097); ZFP42(132625); MRFAP1L1(114932); |
| | | WDR1(9948); DKFZp761B107(91050); SLA/LP(51091); GABRA4 |
| | | (2557); TEC(7006); DHRS8(51170); NR3C2(4306); SFRP2(6423); FGG(2266); |
| | | ADCY2(108); FAM105B(90268); GZMA(3001); RASA1(5921); RGMB |
| | | (285704); HISPPD1(23262); APC(324); PHF15(23338); GM2A(2760); CNOT6 |
| | | (57472); ANKH(56172); PRLR(5618); ST8SIA4(7903); FLJ20125(54826); |
| | | C5orf13(9315); MARCH3(115123); KIAA1961(96459); C5orf15(56951); |
| | | PPP2CA(5515); HNRPA0(10949); APBB3(10307); PPP2R2B(5521); CSF1R |
| | | (1436); LMAN2(10960); DKFZp686I15217(401232); RREB1(6239); GCNT2 |
| | | (2651); E2F3(1871); HIST1H2AC(8334); BTN3A2(11118); PARC(23113); |
| | | KIAA1586(57691); BAG2(9532); PTP4A1(7803); COL19A1(1310); PRDM13 |
| | | (59336); QRSL1(55278); FOXO3A(2309); IGF2R(3482); MUTED(63915); |
| | | ATXN1(6310); HIST1H2BG(8339); BAT3(7917); ELOVL5(60481); |
| | | DST(667); SFT2D1(113402); DACT2(168002); TSPAN13(27075); AHR(196); |
| | | HDAC9(9734); SEPT7(989); DBNL(28988); PHTF2(57157); ARPC1A(10552); |
| | | C7orf19(80228); KDELR2(11014); TWISTNB(221830); IGF2BP3(10643); |
| | | C7orf11(136647); NCF1(4687); FGL2(10875); TRIM4(89122); SYPL1 |
| | | (6856); WNT2(7472); CTTNBP2(83992); SLC35B4(84912); CRYGN(155051); |
| | | C7orf13(129790); DLGAP2(9228); LPL(4023); UNC5D(137970); C8orf1 |
| | | (734); PTDSS1(9791); SDC2(6383); GRHL2(79977); FZD6(8323); BAI1 |
| | | (575); ASAH1(427); PNMA2(10687); CCDC25(55246); UBE2W(55284); TPD52 |
| | | (7163); CNGB3(54714); WDR21C(138009); DERL1(79139); VLDLR |
| | | (7436); UBAP1(51271); MAK10(60560); ANP32B(10541); FREQ(23413); |
| | | GTF3C5(9328); KIAA1815(79956); PSIP1(11168); CDKN2B(1030); SMU1 |
| | | (55234); TLN1(7094); GNE(10020); ZBTB5(9925); SHB(6461); IGFBPL1 |
| | | (347252); TRAF1(7185); GOLGA1(2800); CIZ1(25792); TOR1A(1861); FAM78A |
| | | (286336); SURF5(6837); VAV2(7410); INPP5E(56623); COMMD3(23412); |
| | | PCGF4(648); C10orf9(219771); C10orf70(55847); CHST3(9469); SLK |
| | | (9748); PLEKHA1(59338); INPP5A(3632); SFMBT2(57713); CUL2(8453); |
| | | PPP3CB(5532); ACTR1A(10121); GPAM(57678); C10orf82(143379); EBF3 |
| | | (253738); SCGB1C1(147199); AMPD3(272); EXT2(2132); PPFIA1(8500); |
| | | PPME1(51400); C11orf30(56946); KIAA1377(57562); TMEM24(9854); |
| | | FLJ21103(79607); TPP1(1200); FANCF(2188); LGR4(55366); TRAF6(7189); |
| | | CHST1(8534); ARHGAP1(392); SPTBN2(6712); SHANK2(22941); RA |

Table 4-102

| | | |
|---|---|---|
| | | B6A(5870); HBXAP(51773); MCAM(4162); SCN3B(55800); RAB6IP2(23085); |
| | | PRMT8(56341); CCND2(894); NANOG(79923); FGD4(121512); ARID2 |
| | | (196528); NR4A1(3164); HOXC6(3223); ERBB3(2065); TSPAN31(6302); |
| | | CCT2(10576); USP30(84749); CUTL2(23316); FBXW8(26259); TMEM132B |
| | | (114795); RAN(5901); RAD52(5893); FLJ10292(55110); WBP11(51729); |
| | | SYT10(341359); SLC38A2(54407); AMIGO2(347902); LOC144233(144233); |
| | | KITLG(4254); KCTD10(83892); ANAPC7(51434); C12orf47(51275); |
| | | FGF9(2254); ATP12A(479); ATP8A2(51761); LOC400120(400120); PHF11 |
| | | (51131); GPC6(10082); FLJ25477(219287); SMAD9(4093); KBTBD7(84078); |
| | | POU4F1(5457); VGCNL1(259232); LIG4(3981); RAB20(55647); DCUN1D2 |
| | | (55208); MGC40069(348035); ARHGAP5(394); KLHDC1(122773); |
| | | NRXN3(9369); TRAF3(7187); C14orf124(56948); SNX6(58533); CDKL1 |
| | | (8814); SGPP1(81537); DPF3(8110); ITPK1(3705); BTBD7(55727); C14orf2 |
| | | (9556); TMCO5(145942); SORD(6652); PIAS1(8554); GOLGA(55889); ARID3B |
| | | (10620); IGF1R(3480); MEF2A(4205); NIPA2(81614); SLC12A6(9990); |
| | | RASGRP1(10125); CCPG1(9236); UACA(55075); GOLGA6(342096); |
| | | RASGRF1(5923); BNC1(646); PLIN(5346); TRAF7(84231); PDPK1(5170); |
| | | UBN1(29855); A2BP1(54715); MGC2654(79091); KIAA0350(23274); KIAA0251 |
| | | (23042); EEF2K(29904); PRKCB1(5579); PRRT2(112476); MT2A |
| | | (4502); CYB5B(80777); RPL13(6137); CCDC78(124093); GSPT1(2935); ARL6IP |
| | | (23204); ZNF694(342357); PLLP(51090); C16orf46(123775); COX4NB |
| | | (10328); FBXO31(79791); FAM57A(79850); HIC1(3090); MGC88387(400569); |
| | | LOC374768(374768); SMCR7(125170); WSB1(26118); TRAF4(9618); |
| | | NF1(4763); TADA2L(6871); NAGS(162417); HEXIM2(124790); NXPH3 |
| | | (11248); PRKAR1A(5573); ZZEF1(23140); ANKFY1(51479); GAS7(8522); |
| | | SSH2(85464); MYO1D(4642); OSBPL7(114881); CA10(56934); APPBP2 |
| | | (10513); CD300LB(124599); CD300C(10871); MAFG(4097); ANKRD12 |
| | | (23253); HRH4(59340); SMAD4(4089); FLJ44313(400658); ZFP161(7541); |
| | | SPIRE1(56907); PSTPIP2(9050); SMAD2(4087); PTBP1(5725); UHRF1 |
| | | (29128); LOC126536(126536); UPK1A(11045); SIPA1L3(23094); FOSB(2354); |
| | | CRX(1406); RPL13A(23521); KLP1(57106); ZNF71(58491); ZNF543(125919); |
| | | ZNF547(284306); ZNF549(256051); KIAA0963(22904); LMNB2 |
| | | (84823); FUT6(2528); INSR(3643); TNPO2(30000); LENG9(94059); COX6B2 |
| | | (125965); VN1R1(57191); HARS2(92675); C20orf100(84969); SLC12A5 |
| | | (57468); CSE1L(1434); MOCS3(27304); DNAJC5(80331); MYT1(4661); |
| | | PLAGL2(5326); C20orf112(140688); GGTL3(2686); PRKCBP1(23613); CYP24A1 |
| | | (1591); LAMA5(3911); BTBD4(140685); ABCC13(150000); SH3BGR |
| | | (6450); PKNOX1(5316); ERG(2078); C21orf25(25966); ZNF295(49854); |
| | | C21orf29(54084); CABP7(164633); CBX7(23492); TTLL12(23170); SHOX |
| | | (6473); CLCN4(1183); MBTPS2(51360); NYX(60506); GAGE1(2543); RP6- |
| | | 213H19.1(51765); LOC203547(203547); P2RY8(286530); GPM6B(2824); |
| | | NXF5(55998); MCART6(401612); LAMP2(3920); CD24(934); |
| hsa-miR-558 | 2072; | SAMD11(148398); TP73(7161); DFFB(1677); H6PD(9563); RP13-15M17.2 |
| | | (199953); TNFRSF1B(7133); CDC42(998); LCK(3932); RBBP4(5928); MACF1 |
| | | (23499); PLK3(1263); CDKN2C(1031); CDC14A(8556); RAP1A(5906); |
| | | SLAMF8(56833); UHMK1(127933); C1orf21(81563); PKP1(5317); PPFIA4 |
| | | (8497); ADORA1(134); PRELP(5549); IL24(11009); G0S2(50486); C1orf95 |
| | | (375057); C1orf69(200205); EDARADD(128178); CCNL2(81669); TNFRSF14 |
| | | (8764); DNAJC11(55735); ENO1(2023); DFFA(1676); FRAP1(2475); |
| | | RCC2(55920); TMCO4(255104); LUZP1(7798); E2F2(1870); BSDC1(55108); |
| | | KIAA0319L(79932); OXCT2(64064); RIMS3(9783); MUTYH(4595); |
| | | SLC35D1(23169); GBP4(115361); C1orf183(55924); NOTCH2(4853); SV2A |
| | | (9900); PYGO2(90780); APOBEC4(403314); SYT2(127833); TP53BP2 |
| | | (7159); TMEM63A(9725); DPYSL5(56896); SNX17(9784); FOSL2(2355); BIRC6 |
| | | (57448); TCF7L1(83439); MAL(4118); KCNIP3(30818); PROC(5624); |
| | | ZAK(51776); ALS2CR19(117583); DNAJB2(3300); OSR1(130497); CGREF1 |
| | | (10669); RTN4(57142); EDAR(10913); ANAPC1(64682); PAX8(7849); |
| | | BIN1(274); ERCC3(2071); STAT1(6772); RAPH1(65059); PAX3(5077); WDFY1 |
| | | (57590); CUL3(8452); COL4A4(1286); HDAC4(9759); MTERFD2(130916); |
| | | TTLL3(26140); FANCD2(2177); VHL(7428); MKRN2(23609); NR2C2 |
| | | (7182); PDCD6IP(10015); DLEC1(9940); NKTR(4820); ZNF660(285349); |
| | | RBM5(10181); HEMK1(51409); RBM15B(29890); SIDT1(54847); KALRN |
| | | (8997); ATP2C1(27032); ACPP(55); TP73L(8626); SRGAP3(9901); TADA3L |
| | | (10474); CIDEC(63924); ZFYVE20(64145); ALS2CL(259173); QRICH1 |
| | | (54870); TMEM110(375346); FHIT(2272); RAB43(339122); STAG1(1 |

Table 4-103

0274); TNFSF10(8743); PCGF3(10336); HD(3064); SORCS2(57537); CD38
(952); ANAPC4(29945); IL8(3576); CCNG2(901); NFKB1(4790); TLR2(7097);
GAK(2580); MXD4(10608); LRPAP1(4043); APBB2(323); CCNI(10983);
FAM13A1(10144); MAD2L1(4085); ELF2(1998); SPATA4(132851); CASP3
(836); SKP2(6502); GZMA(3001); RAD17(5884); SCAMP1(9522); APC
(324); TCF7(6932); SLC36A1(206358); SFXN1(94081); NSD1(64324); N4BP3
(23138); UNG2(10309); MCC(4163); SEMA6A(57556); IRF1(3659);
DIAPH1(1729); FGF1(2246); FBXW11(23291); HK3(3101); RIPK1(8737);
TCF19(6941); TNF(7124); CSNK2B(1460); C2(717); ANKS1A(23294); CDKN1A
(1026); PARC(23113); FOXO3A(2309); GRM1(2911); SASH1(23328);
C6orf85(63027); NEDD9(4739); HIST1H2AK(8330); RFP(5987); GABBR1
(2550); MDC1(9656); LTB(4050); DAXX(1616); BAK1(578); C6orf125(84300);
CCND3(896); CUL7(9820); C6orf154(221424); TNFRSF21(27242);
DST(667); SESN1(27244); MAP3K5(4217); MAFK(7975); FOXK1(221937);
AHR(196); HDAC9(9734); KIAA1706(80820); EGFR(1956); CYLN2(7461);
BCAP29(55973); KIAA0265(23008); FLJ11000(55281); CUL1(8454); SNX8
(29886); HOXA1(3198); JAZF1(221895); SCRN1(9805); ELMO1(9844);
ECOP(81552); CALN1(83698); YWHAG(7532); CDK6(1021); ZC3HC1
(51530); FLJ12700(79970); FASTK(10922); ZNF705CP(389631); RHOBTB2
(23221); TNFRSF10C(8794); ESCO2(157570); UNC5D(137970); TACC1
(6867); E2F5(1875); PTDSS1(9791); FZD6(8323); EBAG9(9166); MYC(4609);
CHRAC1(54108); C8orf30A(51236); LRRC14(9684); PINX1(54984);
LZTS1(11178); BIN3(55909); TNFRSF10A(8797); TRIM35(23087); DUSP4
(1846); SFRP1(6422); ANK1(286); MYST3(7994); PDE7A(5150); TP53INP1
(94241); MTSS1(9788); NUDT2(318); C9orf19(152007); DAPK1(1612);
COL27A1(85301); PAPPA(5069); OLFML2A(169611); GPR107(57720); ABL1
(25); BAG1(573); C9orf48(347240); FLJ35740(253650); SEMA4D(10507);
PTCH(5727); ROD1(9991); TRAF1(7185); CIZ1(25792); ASB6(140459);
TSC1(7248); CARD9(64170); ANAPC2(29882); GTPBP4(23560); TSPAN15
(23555); UNC5B(219699); PTEN(5728); KIF11(3832); CYP2C9(1559);
FRAT1(10023); SCD(6319); LZTS2(84445); NOLC1(9221); SUFU(51684);
DOCK1(1793); KNDC1(85442); MTG1(92170); ZWINT(11130); SPOCK2
(9806); DLG5(9231); POLR3A(11128); TMEM10(93377); FRAT2(23401); FAM26C
(255022); MKI67(4288); BNIP3(664); BRSK2(9024); STIM1(6786);
HIPK3(10114); API5(8539); EXT2(2132); DPF2(5977); FKSG44(83786);
LRP5(4041); CCND1(595); BIRC2(329); CUL5(8065); ATM(472); TAGLN
(6876); LOH11CR2A(4013); EI24(9538); FLJ21103(79607); IGF2(3481); ARFIP2
(23647); EIF4G2(1982); CYBASC3(220002); SYT7(9066); MAP3K11
(4296); RELA(5970); FOSL1(8061); BRMS1(25855); CLCF1(23529); PPP1CA
(5499); FGF4(2249); CASP4(837); PPP2R1B(5519); BCL9L(283149);
LTBR(4055); TNFRSF7(939); CNAP1(9918); PTMS(5763); NECAP1(25977);
DDX11(1663); ESPL1(9700); PFDN5(5204); PDE1B(5153); PA2G4(5036);
UBE3B(89910); PRKAB1(5564); KNTC1(9735); ULK1(8408); RAD52
(5893); TNFRSF1A(7132); LRP6(4040); HDAC7A(51564); MCRS1(10445);
FMNL3(91010); FAIM2(23017); DDIT3(1649); DUSP6(1848); DIP13B(55198);
ANAPC7(51434); FAM109A(144717); HRK(8739); FBXO21(23014);
PITPNM2(57605); CDK8(1024); LOC440138(440138); SUGT1(10910); HS6ST3
(266722); ING1(3621); TFDP1(7027); LATS2(26524); FLJ25477(219287);
SLC7A1(6541); FOXO1A(2308); C14orf21(161424); PNN(5411); PSEN1
(5663); VASH1(22846); TRAF3(7187); KIAA0125(9834); ACIN1(22985);
NRL(4901); IPO4(79711); TM9SF1(10548); CIDEB(27141); GZMB(3002);
STRN3(29966); NFKBIA(4792); RAB15(376267); ABCD4(5826); LTBP2
(4053); PGF(5228); NEK9(91754); TGFB3(7043); POMT2(29954); ITPK1
(3705); BAG5(9529); PPP1R13B(23368); LOC440248(440248); BUB1B
(701); CAPN3(825); TMEM62(80021); MAPK6(5597); ARID3B(10620); CSK
(1445); ARNT2(9915); SCAND2(54581); ABHD2(11057); NIPA1(123606);
AVEN(57099); BMF(90427); TTBK2(146057); BNIP2(663); DAPK2(23604);
CSNK1G1(53944); LSM16(80153); DKFZp434I1020(196968); RAB40C
(57799); KIAA1924(197335); KIAA0683(9894); MAPK8IP3(23162); TSC2
(7249); CCNF(899); FLYWCH1(84256); KREMEN2(79412); TNFRSF12A
(51330); DNASE1(1773); GLIS2(84662); MGRN1(23295); UBN1(29855);
MGC2654(79091); PLK1(5347); ATXN2L(11273); CARD15(64127); CX3CL1
(6376); KATNB1(10300); ELMO3(79767); PARD6A(50855); PDPR(55066);
ZNRF1(84937); LOC348180(348180); LOC197322(197322); CCDC78
(124093); NME3(4832); PKMYT1(9088); ERN2(10595); LOC440350(44

Table 4-104

| | | |
|---|---|---|
| | | 0350); Pfs2(51659); PCOLN3(5119); TUSC5(286753); HIC1(3090); GARNL4 |
| | | (23108); ALOX12(239); TNFSF12(8742); PFAS(5198); MAPK7(5598); TNFAIP1 |
| | | (7126); SARM1(23098); NF1(4763); LIG3(3980); NBR2(10230); MAPT |
| | | (4137); NGFR(4804); STXBP4(252983); TLK2(11011); PRKCA(5578); |
| | | MAP2K6(5608); RAB37(326624); TMEM104(54868); CDR2L(30850); CDK3 |
| | | (1018); SPHK1(8877); BIRC5(332); CARD14(79092); BAIAP2(10458); |
| | | GPS1(2873); ABR(29); PITPNA(5306); MNT(4335); NALP1(22861); AIPL1 |
| | | (23746); GPS2(2874); TP53(7157); GAS7(8522); TOM1L2(146691); SPAG5 |
| | | (10615); TBC1D3C(414060); GCN5L2(2648); HDAC5(10014); LRRC59 |
| | | (55379); CUEDC1(404093); SEPT4(5414); ERN1(2081); NT5C(30833); RECQL5 |
| | | (9400); CBX4(8535); TBC1D16(125058); MAFG(4097); MAPK4(5596); |
| | | ST8SIA3(51046); PHLPP(23239); BCL2(596); PARD6G(84552); CSNK1G2 |
| | | (1455); GADD45B(4616); HMG20B(10362); CHAF1A(10036); UHRF1 |
| | | (29128); PIN1(5300); LOC388503(388503); ICAM1(3383); DNM2(1785); |
| | | CASP14(23581); PLEKHF1(79156); APLP1(333); PSMD8(5714); MAP3K10 |
| | | (4294); ARHGEF1(9138); PVR(5817); BCL3(602); FOSB(2354); SPHK2 |
| | | (56848); NALP2(55655); GNG7(2788); AES(166); TNFSF14(8740); ELAVL1 |
| | | (1994); CAPN12(147968); ERF(2077); ERCC2(2068); PPP1R13L(10848); |
| | | BBC3(27113); LIG1(3978); CARD8(22900); KLK10(5655); LOC147808 |
| | | (147808); STK35(140901); C20orf29(55317); MCM8(84515); ITCH(83737); |
| | | CEP250(11190); MYBL2(4605); STK4(6789); CSE1L(1434); PARD6B |
| | | (84612); SS18L1(26039); RTEL1(51750); ZGPAT(84619); TPD52L2(7165); |
| | | PCNA(5111); C20orf112(140688); PTGIS(5740); CTCFL(140690); FLJ90166 |
| | | (164284); CABLES2(81928); DIDO1(11083); SON(6651); MX1(4599); |
| | | KIAA0179(23076); PDXK(8566); TRPM2(7226); DSCAM(1826); PLAC4 |
| | | (191585); SNF1LK(150094); C21orf58(54058); BCL2L13(23786); FLJ42953 |
| | | (400892); HIC2(23119); BCR(613); ADORA2A(135); GAS2L1(10634); |
| | | NF2(4771); CABP7(164633); TCN2(6948); MCM5(4174); TRIOBP(11078); |
| | | MICAL-L1(85377); MGAT3(4248); CACNA1I(8911); EP300(2033); ACO2 |
| | | (50); BIK(638); GTSE1(51512); BID(637); HTF9C(27037); THAP7(80764); |
| | | PPM1F(9647); MN1(4330); MGC17330(113791); IL2RB(3560); SSTR3(6753); |
| | | KIAA1904(114794); CARD10(29775); UNC84B(25777); PDGFB(5155); |
| | | LOC150383(150383); MAPK12(6300); SBF1(6305); LOC440836(440836); |
| | | WWC3(55841); HDAC6(10013); MLLT7(4303); TAF1(6872); DIAPH2 |
| | | (1730); STAG2(10735); RP6-213H19.1(51765); IKBKG(8517); DKC1(1736); |
| | | KAL1(3730); RS1(6247); SMC1L1(8243); BTK(695); HCFC1(3054); MECP2 |
| | | (4204); XX-FW81657B9.4(8270); MTCP1(4515); |
| hsa-miR-561 | 2073; | H6PD(9563); PAX7(5081); CDC42(998); CLIC4(25932); ZBTB8(127557); |
| | | EIF2C4(192670); EIF2C3(192669); THRAP3(9967); MACF1(23499); PRPF38A |
| | | (84950); PRKAA2(5563); MIER1(57708); LPHN2(23266); PRKACB |
| | | (5567); SH3GLB1(51100); HS2ST1(9653); CDC7(8317); CDC14A(8556); SLC30A7 |
| | | (148867); RAP1A(5906); CD2(914); CKS1B(1163); DAP3(7818); PEA15 |
| | | (8682); UHMK1(127933); FMO2(2327); DNM3(26052); RALGPS2(55103); |
| | | C1orf19(116461); RGS18(64407); NEK7(140609); MDM4(4194); G0S2 |
| | | (50486); C1orf107(27042); KCTD3(51133); TGFB2(7042); CIAS1(114548); |
| | | CDC2L2(985); CTNNBIP1(56998); DFFA(1676); FRAP1(2475); CASP9 |
| | | (842); RCC2(55920); CLSPN(63967); TESK2(10420); IPP(3652); EPS15 |
| | | (2060); ITGB3BP(23421); PAP2D(163404); SASS6(163786); NRAS(4893); |
| | | NOTCH2(4853); PRKAB2(5565); RP5-998N21.6(440686); MCL1(4170); PYGO2 |
| | | (90780); PEX19(5824); ASPM(259266); NUCKS1(64710); USH2A(7399); |
| | | TP53BP2(7159); ACBD3(64746); C1orf96(126731); TOMM20(9804); |
| | | SOX11(6664); RHOB(388); PPP1CB(5500); SPAST(6683); BIRC6(57448); |
| | | PPM1B(5495); PRKCE(5581); MSH2(4436); ACTR2(10097); GMCL1(64395); |
| | | KCNIP3(30818); BRRN1(23397); BCL2L11(10018); EPB41L5(57669); |
| | | UBXD2(23190); ACVR2A(92); RIF1(55183); GALNT13(114805); PPIG(9360); |
| | | ZAK(51776); SLC39A10(57181); CASP10(843); CYP20A1(57404); CREB1 |
| | | (1385); HRB(3267); YWHAQ(10971); MATN3(4148); EIF2AK2(5610); |
| | | PRKD3(23683); BCL11A(53335); CCT4(10575); GFPT1(2673); TIA1(7072); |
| | | SEPT10(151011); BUB1(699); ANAPC1(64682); IL1A(3552); ERCC3 |
| | | (2071); FLJ45645(375287); ARL5A(26225); IFIH1(64135); NCKAP1(10787); |
| | | STK17B(9262); HECW2(57520); PGAP1(80055); FZD5(7855); BARD1 |
| | | (580); PAX3(5077); CUL3(8452); HDAC4(9759); FANCD2(2177); PCAF(8850); |
| | | PDCD6IP(10015); CTNNB1(1499); RBM5(10181); APPL(26060); ARL13B |
| | | (200894); ALCAM(214); GTF2E1(2960); ACPP(55); SMC4L1(10051); |
| | | FXR1(8087); SENP2(59343); TP73L(8626); FYTTD1(84248); DAZL(1618 |

Table 4-105

); TRAIP(10293); NEK4(6787); TMF1(7110); FOXP1(27086); EIF4E3(317649);
PROK2(60675); VGLL3(389136); C3orf17(25871); GSK3B(2932); STAG1
(10274); PIK3CB(5291); KPNA4(3840); CENTB2(23527); SLC4A4(8671);
IL8(3576); CCNG2(901); AFF1(4299); FGF2(2247); TRIM2(23321); TLR2
(7097); STOX2(56977); CCNI(10983); SCD5(79966); WDFY3(23001);
UNC5C(8633); DKK2(27123); LEF1(51176); PRSS12(8492); MAD2L1(4085);
GYPA(2993); SFRP2(6423); NEK1(4750); MFAP3L(9848); CASP3(836);
DNAJA5(134218); SKP2(6502); NIPBL(25836); GHR(2690); GZMA(3001);
ERBB2IP(55914); CCNB1(891); RAD17(5884); F2R(2149); SCAMP1
(9522); LNPEP(4012); APC(324); CSNK1G3(1456); MEGF10(84466); SLC12A2
(6558); LOC90624(90624); RAD50(10111); TCF7(6932); PCDHB9(56127);
G3BP(10146); CPEB4(80315); SEMA5A(9037); CTNND2(1501); PRLR(5618);
PRKAA1(5562); MOCS2(4338); BIRC1(4671); ARRDC3(57561);
FLJ25680(134187); ELL2(22936); MCC(4163); TMED7(51014); ATG12
(9140); NME5(8382); GMCL1L(64396); MAPK13(5603); CDKN1A(1026);
VEGF(7422); CDC5L(988); BAG2(9532); PTP4A1(7803); TTK(7272); AMD1
(262); QKI(9444); DHX16(8449); SUPT3H(8464); DST(667); PGM3(5238);
SYNCRIP(10492); UBE2J1(51465); BCLAF1(9774); MAP3K5(4217); PLAGL1
(5325); LATS1(9113); PDCD2(5134); RBAK(57786); AHR(196); SP4
(6671); SEPT7(989); ZNF92(168374); ADAM22(53616); PFTK1(5218); FZD1
(8321); GATAD1(57798); RINT1(60561); BCAP29(55973); TRIAD3(54476);
PMS2(5395); SOSTDC1(25928); CYCS(54205); INHBA(3624); ZNF117
(7670); CDK6(1021); FLJ31818(154743); TFEC(22797); HIPK2(28996);
ZNF705CP(389631); EFHA2(286097); POLR3D(661); BNIP3L(665); ESCO2
(157570); RBM13(84549); UNC5D(137970); FKSG2(59347); BAG4(9530);
TACC1(6867); C8orf4(56862); UBE2V2(7336); SULF1(23213); TERF1
(7013); HNF4G(3174); ZFHX4(79776); EFCBP1(64168); PPM2C(54704);
MTDH(92140); FZD6(8323); EBAG9(9166); MYC(4609); PHF20L1(51105);
CNOT7(29883); MTMR7(9108); PSD3(23362); LZTS1(11178); TNFRSF10B
(8795); TNFRSF10D(8793); TRIM35(23087); WHSC1L1(54904); PLAG1
(5324); PAG1(55824); NBN(4683); CCNE2(9134); TP53INP1(94241);
STK3(6788); TRPS1(7227); RAD21(5885); EXT1(2131); DDEF1(50807); JAK2
(3717); RECK(8434); NTRK2(4915); GADD45G(10912); INVS(27130);
TMEM38B(55151); PALM2(114299); ABL1(25); IFNA2(3440); BAG1(573);
GAS1(2619); OGN(4969); PTCH(5727); TNFSF15(9966); TNFSF8(944);
TRAF1(7185); CIZ1(25792); FNBP1(23048); TSC1(7248); BRD3(8019);
C10orf9(219771); DKK1(22943); PHYHIPL(84457); LRRTM3(347731); SIRT1
(23411); PTEN(5728); FAS(355); KIF11(3832); ENTPD7(57089); NOLC1
(9221); TCF7L2(6934); DOCK1(1793); IRXL1(283078); CUL2(8453);
ERCC6(2074); ZWINT(11130); PPP3CB(5532); KCNMA1(3778); DLG5(9231);
FRAT2(23401); DCLRE1A(9937); MKI67(4288); BNIP3(664); MLSTD2
(84188); GAS2(2620); HIPK3(10114); API5(8539); CRSP6(9440); BIRC3
(330); BIRC2(329); ATM(472); FLJ46266(399949); ARHGEF12(23365);
LOH11CR2A(4013); CHEK1(1111); EIF4G2(1982); WT1(7490); CD59(966);
TRAF6(7189); RELA(5970); SLC36A4(120103); SESN3(143686); CASP5(838);
CASP1(834); PPP2R1B(5519); CDKN1B(1027); FGD4(121512);
LRRK2(120892); MDM2(4193); THAP2(83591); SLC25A3(5250); SCYL2(55681);
GAS2L3(283431); KNTC1(9735); FZD10(11211); RAN(5901); ING4
(51147); CLEC2B(9976); BCAT1(586); KRAS(3845); SLC38A1(81539); HDAC7A
(51564); ZNF641(121274); C12orf22(81566); DDIT3(1649); CDK4
(1019); IGF1(3479); PPP1CC(5501); ANAPC5(51433); MPHOSPH9(10198);
CDK2AP1(8099); HSMPP8(54737); FGF9(2254); TNFRSF19(55504);
BRCA2(675); RFP2(10206); FLJ30707(220108); SUGT1(10910); C13orf6
(84945); ING1(3621); CUL4A(8451); TFDP1(7027); ZDHHC20(253832); CSNK1A1L
(122011); FOXO1A(2308); FBXL3(26224); LIG4(3981); TXNDC
(81542); CGRRF1(10668); C14orf135(64430); PPM1A(5494); PCNX(22990);
STRN3(29966); EGLN3(112399); NIN(51199); NEK9(91754); TMED10
(10972); GTF2A1(2957); CHES1(1112); BTBD7(55727); SPRED1(161742);
BUB1B(701); MAPK6(5597); KIF23(9493); ARIH1(25820); FBXO22(26263);
TJP1(7082); BMF(90427); MYEF2(50804); BCL2L10(10017); PYGO1
(26108); PRTG(283659); VPS13C(54832); PRO0149(29035); RBL2(5934);
CTCF(10664); CARHSP1(23589); ERN2(10595); NUDT21(11051); CIAPIN1
(57019); CSNK2A2(1459); SMPD3(55512); PCOLN3(5119); ALOX12(239);
FAM18B(51030); NLK(51701); NF1(4763); CROP(51747); PPM1D(8493);
PRKAR1A(5573); SLC9A3R1(9368); ANKFY1(51479); TP53(7157); G

Table 4-106

| | | |
|---|---|---|
| | | AS7(8522); NUFIP2(57532); BECN1(8678); BRCA1(672); SFRS1(6426); PTRH2 |
| | | (51651); APPBP2(10513); ERN1(2081); HELZ(9931); SOCS3(9021); |
| | | ANKRD12(23253); GNAL(2774); CABLES1(91768); CCDC5(115106); SMAD4 |
| | | (4089); DCC(1630); MALT1(10892); PHLPP(23239); ROCK1(6093); |
| | | BCL2(596); CDH19(28513); PARD6G(84552); ILF3(3609); ZNF441(126068); |
| | | ZNF430(80264); ZNF493(284443); C19orf2(8725); AXL(558); APOE(348); |
| | | FOSB(2354); BCL2L12(83596); ZFP28(140612); ZNF100(163227); ZNF545 |
| | | (284406); TGFB1(7040); ERF(2077); PPP1R13L(10848); CARD8(22900); |
| | | ZNF83(55769); MCM8(84515); KIAA1219(57148); STK4(6789); CSE1L |
| | | (1434); PARD6B(84612); CSNK2A1(1457); TXNDC13(56255); RBM12 |
| | | (10137); AURKA(6790); SYCP2(10388); DIDO1(11083); USP16(10600); |
| | | SON(6651); CHAF1B(8208); C21orf91(54149); SNF1LK(150094); APOL6 |
| | | (80830); TNRC6B(23112); GTSE1(51512); MN1(4330); CHEK2(11200); DMC1 |
| | | (11144); TOB2(10766); MBTPS2(51360); CCNB3(85417); MLLT7(4303); |
| | | PCDH11X(27328); NRK(203447); ATG4A(115201); AGTR2(186); BIRC4 |
| | | (331); STAG2(10735); RP6-213H19.1(51765); PPP2R3B(28227); FIGF |
| | | (2277); TAB3(257397); DMD(1756); CRSP2(9282); AMMECR1(9949); CUL4B |
| | | (8450); RBMX(27316); CETN2(1069); PCDH11Y(83259); USP9Y(8287); |
| | | NLGN4Y(22829); DAZ2(57055); DAZ4(57135); DAZ3(57054); |
| hsa-miR-565 | 2074; | PRKCZ(5590); TP73(7161); TNFRSF1B(7133); PAX7(5081); EPHA8(2046); |
| | | SEPN1(57190); CCDC21(64793); NUDC(10726); MACF1(23499); PPIE |
| | | (10450); ST3GAL3(6487); NFIA(4774); CDC14A(8556); GPR88(54112); |
| | | CSF1(1435); DCLRE1B(64858); HIPK1(204851); CD2(914); BCL9(607); BNIPL |
| | | (149428); PSMD4(5710); PEA15(8682); TOMM40L(84134); XCL1(6375); |
| | | FAM20B(9917); RGS2(5997); NAV1(89796); PPP1R12B(4660); CNTN2 |
| | | (6900); PCTK3(5129); SRGAP2(23380); IL24(11009); TRAF5(7188); TGFB2 |
| | | (7042); C1orf121(51029); CIAS1(114548); TNFRSF18(8784); CCNL2 |
| | | (81669); CDC2L2(985); TNFRSF14(8764); KIAA0495(57212); CTNNBIP1 |
| | | (56998); DFFA(1676); FRAP1(2475); MTHFR(4524); ZBTB17(7709); FBXO42 |
| | | (54455); RCC2(55920); YARS(8565); KIAA0319L(79932); MYCL1 |
| | | (4610); MUTYH(4595); FAF1(11124); CC2D1B(200014); NOTCH2(4853); |
| | | ADAR(103); DEDD(9191); XCL2(6846); MYOC(4653); NMNAT2(23057); |
| | | ASPM(259266); KIF21B(23046); ARL8A(127829); PLEKHA6(22874); TP53BP2 |
| | | (7159); MAPRE3(22924); FOSL2(2355); PPP1CB(5500); LBH(81606); |
| | | BIRC6(57448); ANTXR1(84168); CCT7(10574); TCF7L1(83439); ATOH8 |
| | | (84913); KCNIP3(30818); BRRN1(23397); BCL2L11(10018); POTE2(445582); |
| | | TAIP-2(80034); MAP1D(254042); CD28(940); COL4A3(1285); PSMD1 |
| | | (5707); ASB1(51665); GPC1(2817); BOK(666); HS1BP3(64342); PPM1G |
| | | (5496); EIF2AK2(5610); CCT4(10575); TGFA(7039); EDAR(10913); BUB1 |
| | | (699); ANAPC1(64682); PAX8(7849); FLJ20297(55627); MCM6(4175); |
| | | IFIH1(64135); STAT1(6772); FZD5(7855); IGFBP5(3488); XLF(79840); ARL4C |
| | | (10123); HDAC4(9759); OGG1(4968); FANCD2(2177); SLC6A6(6533); |
| | | OXNAD1(92106); PCAF(8850); CMTM7(112616); MLH1(4292); CTDSPL |
| | | (10217); DLEC1(9940); LIMD1(8994); HEMK1(51409); C3orf52(79669); |
| | | NEK11(79858); KNG1(3827); TP73L(8626); OPA1(4976); RAF1(5894); THRB |
| | | (7068); AXUD1(64651); CCDC13(152206); CCR1(1230); NME6(10201); |
| | | TRAIP(10293); RASSF1(11186); CISH(1154); SEMASG(56920); NT5DC2 |
| | | (64943); TMEM110(375346); ASB14(142686); LRIG1(26018); UBE1C |
| | | (9039); GSK3B(2932); MGLL(11343); RAB43(339122); STAG1(10274); SLAH2 |
| | | (6478); WHSC1(7468); HD(3064); C1QTNF7(114905); CD38(952); ANAPC4 |
| | | (29945); PAICS(10606); DCAMKL2(166614); SLC26A1(10861); LETM1 |
| | | (3954); FLJ46481(389197); HNRPDL(9987); LEF1(51176); FLJ23191 |
| | | (79625); ANAPC10(10393); KIAA1909(153478); CARD6(84674); ERBB2IP |
| | | (55914); RAD17(5884); APC(324); IL13(3596); TCF7(6932); PCDHGB4 |
| | | (8641); MST150(85027); UNC5A(90249); NSD1(64324); ZDHHC11(79844); |
| | | PRLR(5618); UNG2(10309); F2RL2(2151); MCC(4163); ATG12(9140); IRF1 |
| | | (3659); PPP2CA(5515); CDC23(8697); CDC25C(995); DPYSL3(1809); CSNK1A1 |
| | | (1452); ADAM19(8728); FBXW11(23291); HK3(3101); MGAT4B |
| | | (11282); RIPK1(8737); LY86(9450); RREB1(6239); E2F3(1871); LTA(4049); |
| | | TNF(7124); SKIV2L(6499); PACSIN1(29993); ANKS1A(23294); SCUBE3 |
| | | (222663); MDFI(4188); PARC(23113); SLC25A27(9481); PTP4A1(7803); FOXO3A |
| | | (2309); TNFAIP3(7128); NEDD9(4739); DHX16(8449); MDC1(9656); |
| | | TAPBP(6892); C6orf106(64771); TEAD3(7005); FKBP5(2289); MDGA1 |
| | | (266727); TNFRSF21(27242); TRAM2(9697); DST(667); ZBTB24(9841); |
| | | SGK(6446); AGPAT4(56895); IQCE(23288); WIPI2(26100); HDAC9(9734 |

Table 4-107

); DKFZp761I2123(83637); ADCY1(107); EGFR(1956); CYLN2(7461); ADAM22
(53616); FZD1(8321); TRRAP(8295); STAG3(10734); MGC35361(222234);
BCAP29(55973); CASP2(835); CUL1(8454); LOC155060(155060);
ATP6V0E2L(155066); DPP6(1804); MAD1L1(8379); GNA12(2768); TRIAD3
(54476); PMS2(5395); SOSTDC1(25928); HOXA3(3200); CARD4(10392);
ELMO1(9844); CAMK2B(816); TMED4(222068); IGFBP3(3486); STX1A
(6804); HIP1(3092); CDK6(1021); FLJ42280(401388); NAPE-PLD(222236);
FLJ36031(168455); FLJ31818(154743); ZC3HC1(51530); HIPK2(28996);
PTK2B(2185); SCARA3(51435); ELP3(55140); MYC(4609); ZFP41(286128);
C8orf30A(51236); MTUS1(57509); LZTS1(11178); BIN3(55909); TNFRSF10B
(8795); TNFRSF10A(8797); ADRA1A(148); CLU(1191); DUSP4
(1846); SFRP1(6422); RAD21(5885); EXT1(2131); MTSS1(9788); ZNF406
(57623); CYP11B2(1585); ANKRD15(23189); JAK2(3717); MTAP(4507);
RP11-255A11.18(441459); UNC13B(10497); DAPK1(1612); C9orf47(286223);
SLC31A2(1318); NEK6(10783); CDK9(1025); DNM1(1759); COQ4
(51117); C9orf74(81605); ABL1(25); NUP214(8021); WDR5(11091); RXRA
(6256); NDOR1(27158); CDKN2A(1029); C9orf25(203259); UNQ470(375704);
FANCG(2189); GAS1(2619); SEMA4D(10507); DIRAS2(54769); PTCH
(5727); TNFSF15(9966); TRAF1(7185); GOLGA1(2800); CIZ1(25792); TOR1A
(1861); RAPGEF1(2889); TSC1(7248); ABO(28); LCN6(158062); RP11-
350O14.10(389816); ZNF488(118738); UNC5B(219699); SFTPA1(6435);
TSPAN14(81619); PCDH21(92211); CNNM1(26507); SEMA4G(57715);
NOLC1(9221); SUFU(51684); PDCD11(22984); BAG3(9531); KNDC1(85442);
LARP5(23185); C10orf97(80013); MARCH8(220972); ZWINT(11130);
LRRC20(55222); DLG5(9231); FBXW4(6468); ARL3(403); FGFR2(2263);
OAT(4942); MKI67(4288); FLJ46300(399827); SPRN(503542); STIM1(6786);
WEE1(7465); SPON1(10418); GAS2(2620); HIPK3(10114); CRY2(1408);
MDK(4192); F2(2147); C11orf49(79096); TMEM132A(54972); INCENP
(3619); LGALS12(85329); POLA2(23649); SSSCA1(10534); SIPA1(6494);
OVOL1(5017); MUS81(80198); RPS6KB2(6199); CCND1(595); SLCO2B1
(11309); PHCA(55331); ATM(472); CD3E(916); SLC37A2(219855); CHEK1
(1111); ADAMTS15(170689); TOLLIP(54472); DUSP8(1850); IGF2(3481);
CDKN1C(1028); EIF4G2(1982); WT1(7490); CD59(966); ACP2(53); SYT7
(9066); RAB3IL1(5866); CHRM1(1128); MAP3K11(4296); RELA(5970);
AYP1(84153); DKFZp761E198(91056); CLCF1(23529); PPP1CA(5499);
FGF3(2248); SHANK2(22941); RAB30(27314); FZD4(8322); PPP2R1B(5519);
IL18(3606); ZW10(9183); SCN2B(6327); BCL9L(283149); B3GAT1(27087);
ADIPOR2(79602); CCND2(894); TNFRSF7(939); PTPN6(5777); BCL2L14
(79370); CDKN1B(1027); KIAA1467(57613); ESPL1(9700); GDF11
(10220); ERBB3(2065); APAF1(317); GAS2L3(283431); OAS2(4939); FBXW8
(26259); TCF1(6927); KNTC1(9735); HIP1R(9026); NOL1(4839); HDAC7A
(51564); VDR(7421); MCRS1(10445); FAIM2(23017); CBX5(23468);
LOC56901(56901); CTDSP2(10106); WIF1(11197); DUSP6(1848); GALNT4
(8693); BTG1(694); PCTK2(5128); MSI1(4440); C12orf43(64897); CAMKK2
(10645); MPHOSPH9(10198); SCARB1(949); TNFRSF19(55504);
LOC440138(440138); HS6ST3(266722); PHGDHL1(337867); LATS2(26524);
CSNK1A1L(122011); FOXO1A(2308); LIG4(3981); ACTBL1(23784);
BCL2L2(599); RNF31(55072); CDKN3(1033); JDP2(122953); KIAA0329
(9895); TRAF3(7187); KIAA0125(9834); ACIN1(22985); CIDEB(27141); STRN3
(29966); SPTB(6710); MAP3K9(4293); LTBP2(4053); PGF(5228); NEK9
(91754); ZDHHC22(283576); POMT2(29954); THSD3(145501); CHES1
(1112); ITPK1(3705); PRIMA1(145270); PPP1R13B(23368); AKT1(207);
JAG2(3714); SCG3(29106); MAPK6(5597); KIF23(9493); CSK(1445); ZSCAN2
(54993); SLC28A1(9154); AKAP13(11214); ST8SIA2(8128); ASB7
(140460); BMF(90427); CSNK1G1(53944); PDCD7(10081); STRA6(64220);
ACSBG1(23205); BCL2A1(597); MPG(4350); RAB40C(57799); KIAA1924
(197335); TBL3(10607); TSC2(7249); CCNF(899); DNASE1(1773); MGRN1
(23295); PMM2(5373); KIAA0350(23274); C16orf45(89927); EEF2K(29904);
PLK1(5347); CARD15(64127); CYLD(1540); RBL2(5934); KATNB1
(10300); HSPC065(29070); FLJ21736(79984); ELMO3(79767); CTCF(10664);
CDT1(81620); AXIN1(8312); CCDC78(124093); C16orf28(65259); TCEB2
(6923); PKMYT1(9088); FAM100A(124402); XYLT1(64131); GGA2(23062);
KIAA1970(124454); TUFM(7284); MAPK3(5595); TBC1D10B(26000);
SEPT1(1731); PYCARD(29108); SIAH1(6477); TK2(7084); CMTM4(146223);
MGC4655(84752); TRADD(8717); RBM35B(80004); SMPD3(555

Table 4-108

| | | |
|---|---|---|
| | | 12); TERF2(7014); ADAT1(23536); ADAMTS18(170692); FLJ12998(64779); |
| | | CBFA2T3(863); PCOLN3(5119); PAFAH1B1(5048); TNFSF12(8742); |
| | | MPDU1(9526); NTN1(9423); SARM1(23098); NF1(4763); MLLT6(4302); |
| | | STARD3(10948); FZD2(2535); NMT1(4836); IGF2BP1(10642); NGFR(4804); |
| | | ITGA3(3675); TMEM92(162461); CROP(51747); YPEL2(388403); WDR68 |
| | | (10238); MAP3K3(4215); CACNG1(786); SLC9A3R1(9368); CDK3 |
| | | (1018); CARD14(79092); FLJ35220(294131); GPS1(2873); ABR(29); MNT |
| | | (4335); METT10D(79066); KIAA0664(23277); NALP1(22861); GPS2(2874); |
| | | GAS7(8522); TOM1L2(146691); MFAP4(4239); SPAG5(10615); C17orf63 |
| | | (55731); TIAF1(9220); GAS2L2(246176); BECN1(8678); BRCA1(672); HOXB13 |
| | | (10481); PHB(5245); MPO(4353); LIMD2(80774); ERN1(2081); SLC39A11 |
| | | (201266); EXOC7(23265); UBE2O(63893); SOCS3(9021); TBC1D16 |
| | | (125058); FLJ46026(400627); MGC15523(124565); CIDEA(1149); CABLES1 |
| | | (91768); MAPRE2(10982); MAPK4(5596); DCC(1630); ZCCHC2(54877); |
| | | COLEC12(81035); ZBTB7C(201501); BCL2(596); ATP5D(513); DAZAP1 |
| | | (26528); SF3A2(8175); FZR1(51343); GIPC3(126326); P2RY11(5032); |
| | | DNM2(1785); NFIX(4784); CASP14(23581); AP1M1(8907); GLT25D1(79709); |
| | | PIK3R2(5296); UBA52(7311); KLHL26(55295); CCNE1(898); LRP3 |
| | | (4037); KCTD15(79047); KIAA0355(9710); PSMD8(5714); AXL(558); ARHGEF1 |
| | | (9138); APOE(348); SPHK2(56848); PRMT1(3276); MBD3(53615); |
| | | ATP8B3(148229); DAPK3(1613); RANBP3(8498); TNFSF14(8740); PRAM1 |
| | | (84106); PRDX2(7001); DEDD2(162989); PPP1R13L(10848); BBC3(27113); |
| | | LIG1(3978); FUT1(2523); ZCCHC3(85364); TRIB3(57761); PTPRA |
| | | (5786); CDC25B(994); PLCB1(23236); TM9SF4(9777); POFUT1(23509); |
| | | ASXL1(171023); CBFA2T2(9139); TP53INP2(58476); MMP24(10893); CEP250 |
| | | (11190); SRC(6714); PPP1R16B(26051); STK4(6789); SLC9A8(23315); |
| | | ZNF313(55905); TH1L(51497); FLJ44790(400950); ARFGAP1(55738); |
| | | OPRL1(4987); SIRPB1(10326); GFRA4(64096); E2F1(1869); ZHX3(23051); |
| | | CABLES2(81928); DIDO1(11083); STMN3(50861); USP16(10600); SON |
| | | (6651); PKNOX1(5316); COL18A1(80781); SETD4(54093); DSCR3(10311); |
| | | C21orf93(246704); S100B(6285); DGCR8(54487); ZDHHC8(29801); |
| | | LZTR1(8216); HIC2(23119); PPIL2(23759); KIAA0376(23384); ADORA2A |
| | | (135); MYO18B(84700); NF2(4771); LIMK2(3985); YWHAH(7533); MCM5 |
| | | (4174); APOL6(80830); LGALS1(3956); SYNGR1(9145); EP300(2033); |
| | | SEPT3(55964); PPARA(5465); GTSE1(51512); LOC128977(128977); YPEL1 |
| | | (29799); MAPK1(5594); PPM1F(9647); MN1(4330); CARD10(29770); TOB2 |
| | | (10766); CTA-126B4.3(27341); SULT4A1(25830); RP11-191L9.1(400932); |
| | | APXL(357); OGT(8473); IL13RA1(3597); BIRC4(331); XPNPEP2(7512); |
| | | CD40LG(959); AFF2(2334); SLC6A8(6535); IKBKG(8517); SMC1L1 |
| | | (8243); RP11-493K23.2(340529); CHM(1121); BTK(695); SEPT6(23157); CUL4B |
| | | (8450); CD99L2(83692); HCFC1(3054); |
| hsa-miR-570 | 2075; | DFFB(1677); PRDM2(7799); CDC42(998); ARID1A(8289); NUDC(10726); |
| | | WDTC1(23038); RCC1(1104); HDAC1(3065); RBBP4(5928); S100PBP(64766); |
| | | MACF1(23499); CDC20(991); PTPRF(5792); BTF3L4(91408); ZYG11B |
| | | (79699); PRKAA2(5563); GIPC2(54810); SH3GLB1(51100); CDC7(8317); |
| | | HIAT1(64645); CDC14A(8556); FAM102B(284611); RAP1A(5906); DCLRE1B |
| | | (64858); SYCP1(6847); BCL9(607); BNIPL(149428); CKS1B(1163); |
| | | PEA15(8682); UHMK1(127933); POGK(57645); RALGPS2(55103); C1orf21 |
| | | (81563); NAV1(89796); MDM4(4194); IL24(11009); G0S2(50486); TRAF5 |
| | | (7188); TGFB2(7042); RAB4A(5867); C1orf124(83932); ZNF238(10472); |
| | | CCNL2(81669); GNB1(2782); TNFRSF14(8764); TNFRSF9(3604); FBXO42 |
| | | (54455); RCC2(55920); TTMB(399474); FOXJ3(22887); SPATA6(54558); |
| | | SLC35D1(23169); GPR177(79971); FUBP1(8880); BCL10(8915); GBP4 |
| | | (115361); EVI5(7813); TMED5(50999); CLCC1(23155); RSBN1(54665); |
| | | NRAS(4893); NOTCH2(4853); FMO5(2330); MCL1(4170); PYGO2(90780); |
| | | KIAA0907(22889); ASTN(460); IVNS1ABP(10625); GLRX2(51022); ASPM |
| | | (259266); NUCKS1(64710); IL10(3586); C1orf48(25936); C1orf140(400804); |
| | | TP53BP2(7159); ERO1LB(56605); SOX11(6664); RHOB(388); LBH |
| | | (81606); BIRC6(57448); STON1(11037); SPTBN1(6711); GKN1(56287); ANTXR1 |
| | | (84168); HK2(3099); MRPL19(9801); ANKRD36(375248); EIF5B(9669); |
| | | SLC9A2(6549); BCL2L11(10018); KCNJ3(3760); TAIP-2(80034); ZAK |
| | | (51776); HNRPA3(220988); DUSP19(142679); PMS1(5378); SLC39A10 |
| | | (57181); FZD7(8324); ALS2CR13(150864); ABI2(10152); CD28(940); PIP5K3 |
| | | (200576); COL4A3(1285); HRB(3267); SEPT2(4735); RNASEH1(246243); |
| | | E2F6(1876); ASXL2(55252); PPM1G(5496); SRD5A2(6716); CDC42 |

Table 4-109

EP3(10602); RTN4(57142); BCL11A(53335); GFPT1(2673); TIA1(7072); EDAR
(10913); BUB1(699); BIN1(274); ARL5A(26225); CACNB4(785); TLK1
(9874); SLC40A1(30061); PGAP1(80055); SATB2(23314); TRAK2(66008);
RAPH1(65059); BARD1(580); PAX3(5077); HDAC4(9759); EDEM1(9695);
FANCD2(2177); VHL(7428); SLC6A6(6533); PDCD6IP(10015); CTNNB1
(1499); NKTR(4820); ZNF662(389114); SNRK(54861); LIMD1(8994); TDGF1
(6997); ARIH2(10425); RBM15B(29890); APPL(26060); PCNP(57092);
ZPLD1(131368); CD96(10225); EAF2(55840); PLXNA1(5361); NEK11
(79858); ACPP(55); FAIM(55179); SMC4L1(10051); PRKCI(5584); FNDC3B
(64778); KCNMB2(10242); FXR1(8087); SENP2(59343); OPA1(4976);
VGLL4(9686); SLC4A7(9497); EPM2AIP1(9852); PRKAR2A(5576); NEK4
(6787); C3orf63(23272); PRICKLE2(166336); MAGI1(9223); VGLL3(389136);
GCET2(257144); ZBTB20(26137); KPNA1(3836); STAG1(10274); SSR3
(6747); MASP1(5648); PCGF3(10336); KIAA1530(57654); WHSC1(7468);
HD(3064); LOC93622(93622); MED28(80306); HCAP-G(64151); RBPSUH
(3516); STIM2(57620); C4orf12(404201); AFF1(4299); PDLIM5(10611);
NFKB1(4790); FGF2(2247); PLK4(10733); PCDH10(57575); TLR2(7097);
CLCN3(1182); STOX2(56977); GAK(2580); APBB2(323); GABRA4(2557);
UGT2A3(79799); CCNI(10983); PAQR3(152559); WDFY3(23001); LEF1
(51176); CASP6(839); OTUD4(54726); SFRP2(6423); SPATA4(132851); VEGFC
(7424); FLJ11193(55322); IL7R(3575); SKP2(6502); NIPBL(25836);
CARD6(84674); R7BP(401190); FLJ13611(80006); ERBB2IP(55914); SFRS12
(140890); CCNB1(891); F2R(2149); APC(324); DCP2(167227); CSNK1G3
(1456); RAD50(10111); PCDHB9(56127); SLC26A2(1836); G3BP(10146);
LSM11(134353); LOC153222(153222); GOLPH3(64083); PRLR(5618);
PDE4D(5144); BIRC1(4671); MEF2C(4208); LYSMD3(116068); ARRDC3
(57561); FLJ25680(134187); STARD4(134429); MCC(4163); TRIM36(55521);
FEM1C(56929); ATG12(9140); SEMA6A(57556); PPP2CA(5515); KLHL3
(26249); CDC23(8697); DNAJC18(202052); HDAC3(8841); GNPDA1
(10007); CSNK1A1(1452); FBXW11(23291); DUSP1(1843); DDX41(51428);
GRM6(2916); PRPF4B(8899); LY86(9450); JARID2(3720); E2F3(1871);
AIF1(199); CSNK2B(1460); MAPK13(5603); CDKN1A(1026); RNF8(9025);
PARC(23113); VEGF(7422); COL19A1(1310); TTK(7272); FOXO3A(2309);
TBPL1(9519); TNFAIP3(7128); HECA(51696); NEDD9(4739); IER3(8870);
DAXX(1616); CCND3(896); SUPT3H(8464); MCM3(4172); TRAM2
(9697); DST(667); TMEM30A(55754); SESN1(27244); CDC2L6(23097); GOPC
(57120); SERINC1(57515); STX7(8417); BCLAF1(9774); MAP7(9053);
MAP3K5(4217); PLAGL1(5325); SOD2(6648); PDCD2(5134); FOXK1(221937);
TMEM106B(54664); ARL4A(10124); AHR(196); HDAC9(9734); CREB5
(9586); Ells1(222166); ZNRF2(223082); STK17A(9263); AUTS2(26053);
FKBP6(8468); PFTK1(5218); FZD1(8321); LMTK2(22853); RINT1(60561);
HBP1(26959); CBLL1(79872); CASP2(835); CNTNAP2(26047); CUL1
(8454); RBM33(155435); PAPOLB(56903); TRIAD3(54476); PSCD3(9265);
ZNF12(7559); ETV1(2115); SOSTDC1(25928); CARD4(10392); SFRP4
(6424); INHBA(3624); CALN1(83698); YWHAG(7532); CDK6(1021); MCM7
(4176); PPP1R3A(5506); TSGA14(95681); CLN8(2055); BNIP3L(665);
ESCO2(157570); FZD3(7976); UNC5D(137970); SPFH2(11160); BAG4(9530);
TACC1(6867); RAB2(5862); MTFR1(9650); SGK3(23678); SULF1(23213);
ZFHX4(79776); E2F5(1875); RIPK2(8767); EFCBP1(64168); SLC26A7
(115111); FZD6(8323); EBAG9(9166); WISP1(8840); ZNF623(9831); LRRC14
(9684); TNFRSF10B(8795); TNFRSF10A(8797); TRIM35(23087); WHSC1L1
(54904); SFRP1(6422); NCOA2(10499); PAG1(55824); WDR21C
(138009); NBN(4683); RBM12B(389677); TP53INP1(94241); STK3(6788);
TRPS1(7227); RAD21(5885); ANKRD15(23189); JAK2(3717); NUDT2(318);
PCSK5(5125); PSAT1(29968); NTRK2(4915); MAK10(60560); DAPK1
(1612); C9orf47(286223); GADD45G(10912); TGFBR1(7046); NR4A3(8013);
OLFML2A(169611); CDK9(1025); FAM29A(54801); CDKN2B(1030); C9orf72
(203228); TAF1L(138474); BAG1(573); CNTNAP3(79937); ZA20D2
(7763); SEMA4D(10507); PTCH(5727); ANKS6(203286); ROD1(9991); TNFSF8
(944); FBXW2(26190); LHX6(26468); ZBTB26(57684); STRBP(55342);
CUGBP2(10659); PTER(9317); C10orf9(219771); SIRT1(23411); EIF4EBP2
(1979); SGPL1(8879); PTEN(5728); TNKS2(80351); ENTPD7(57089);
BTRC(8945); NOLC1(9221); SUFU(51684); ACADSB(36); DOCK1(1793);
DIP2C(22982); CUL2(8453); MARCH8(220972); SYT15(83849); ZWINT
(11130); SPOCK2(9806); P4HA1(5033); KCNMA1(3778); DLG5(9231); F

Table 4-110

RAT2(23401); FGF8(2253); SMNDC1(10285); GPAM(57678); DCLRE1A
(9937); C10orf118(55088); C10orf46(143384); TIAL1(7073); MKI67(4288);
STIM1(6786); WEE1(7465); MLSTD2(84188); NAV2(89797); GAS2(2620);
HIPK3(10114); GPIAP1(4076); DPF2(5977); CCND1(595); FADD(8772);
BIRC3(330); CUL5(8065); ATM(472); MLL(4297); CBL(867); LOH11CR2A
(4013); CHEK1(1111); RNF141(50862); EIF4G2(1982); DKK3(27122); WT1
(7490); FBXO3(26273); PHF21A(51317); CUGBP1(10658); ZF(58487);
FZD4(8322); SLC36A4(120103); SESN3(143686); CASP1(834); PPP2R1B
(5519); BCL9L(283149); CCND2(894); CNAP1(9918); PTMS(5763); BCL2L14
(79370); H2AFJ(55766); IAPP(3375); ETNK1(55500); STK38L(23012);
TEGT(7009); LARP4(113251); PDE1B(5153); DYRK2(8445); MDM2(4193);
CPSF6(11052); SOCS2(8835); GAS2L3(283431); KIAA1853(84530); TMEM132B
(114795); RAN(5901); EP400(57634); ING4(51147); LRP6(4040);
BCAT1(586); KRAS(3845); TMTC1(83857); FAM60A(58516); DDX23(9416);
FAIM2(23017); BAZ2A(11176); CTDSP2(10106); PHLDA1(22822); SLC6A15
(55117); EEA1(8411); NT5DC3(51559); GIT2(9815); PPP1CC(5501);
ANAPC5(51433); DIABLO(56616); CDK2AP1(8099); CHFR(55743);
HSMPP8(54737); ZNF198(7750); FGF9(2254); PAN3(255967); AKAP11(11215);
RB1(5925); RFP2(10206); RAP2A(5911); ING1(3621); CUL4A(8451);
TFDP1(7027); CDC16(8881); LATS2(26524); FOXO1A(2308); KBTBD6
(89890); KPNA3(3839); KCTD12(115207); C13orf10(64062); DZIP1(22873);
LIG4(3981); C14orf92(9878); BCL2L2(599); MIPOL1(145282); PNN(5411);
TXNDC(81542); PSEN1(5663); GPR65(8477); TRAF3(7187); EIF5(1983);
STRN3(29966); EGLN3(112399); NIN(51199); KIAA0831(22863); EXOC5
(10640); SGPP1(81537); LTBP2(4053); NEK9(91754); SPTLC2(9517);
CHES1(1112); DICER1(23405); BAG5(9529); PPP1R13B(23368); MKRN3
(7681); KLF13(51621); GREM1(26585); SPRED1(161742); BUB1B(701);
GANC(2595); FGF7(2252); GLDN(342035); TMOD2(29767); MAPK6(5597);
CCNB2(9133); RAB8B(51762); PIAS1(8554); FEM1B(10116); KIF23(9493);
THSD4(79875); HMG20A(10363); IGF1R(3480); TJP1(7082); SLC12A6
(9990); TTBK2(146057); MYEF2(50804); C15orf15(51187); BNIP2(663);
CSNK1G1(63944); PDCD7(10081); MESDC2(23184); CHSY1(22856); CLUAP1
(23059); SNN(8303); PAPD5(64282); RBL2(5934); NOL3(8996); CTCF
(10664); SLC7A6(9057); CYB5B(80777); NFAT5(10725); KIAA0174
(9798); AKIN1(8312); C16orf28(65259); GRIN2A(2903); GSPT1(2935); P8
(26471); SIAH1(6477); CSNK2A2(1459); APPBP1(8883); SMPD3(55512);
TXNL4B(54957); MAF(4094); MPHOSPH6(10200); PAFAH1B1(5048); GARNL4
(23108); NLK(51701); TRAF4(9618); GOSR1(5927); NF1(4763); CCL2
(6347); LASP1(3927); FZD2(2535); MAPT(4137); NGFR(4804); CROP
(51747); YPEL2(388403); PRKCA(5578); MAP2K6(5608); BIRC5(332); MNT
(4335); METT10D(79066); AURKB(9212); GAS7(8522); SPAG5(10615);
PHF12(57649); NUFIP2(57532); BRCA1(672); CCDC43(124808); GJA7
(10052); MTMR4(9110); GNA13(10672); AXIN2(8313); HELZ(9931); CSNK1D
(1453); ANKRD12(23253); DTNA(1837); SLC14A1(6563); KIAA0427
(9811); ZFP161(7541); SMAD2(4087); TXNL1(9352); BCL2(596); MBP(4155);
PIK3R2(5296); UBA52(7311); CCNE1(898); DPY19L3(147991); ARHGEF1
(9138); APOE(348); BCL2L12(83596); MYADM(91663); NALP2(55655);
LOC390980(390980); ZNF17(7565); CDC37(11140); DNASE2(1777);
ZNF537(57616); ZNF585A(199704); DPF1(8193); PPP1R13L(10848); CARD8
(22900); KLK10(5655); DKFZp686I1569(400720); MCM8(84515); PLCB1
(23236); CEP250(11190); STK4(6789); CSE1L(1434); PARD6B(84612);
RAB22A(57403); STX16(8675); E2F1(1869); RBL1(5933); SYCP2(10388);
CABLES2(81928); DIDO1(11083); SLC5A3(6526); APP(351); BRWD1(54014);
S100B(6285); KIAA0376(23384); CTB-1048E9.5(402055); KREMEN1
(83999); NF2(4771); APOL6(80830); KDELR3(11015); TNRC6B(23112);
BIK(638); BID(637); YPEL1(29799); MAPK1(5594); MN1(4330); CHEK2
(11200); DMC1(11144); NHP2L1(4809); TAF1(6872); PGK1(5230); DIAPH2
(1730); NGFRAP1(27018); CXorf39(139231); AGTR2(186); BIRC4(331);
STAG2(10735); CD40LG(959); AFF2(2334); DKC1(1736); SPRY3(10251);
KAL1(3730); FIGF(2277); KLHL15(80311); WDR42B(139425); SMC1L1
(8243); WNK3(65267); RP11-130N24.1(340533); ATRX(546); TAF9B(51616);
PCDH19(57526); AMMECR1(9949); DCX(1641); CUL4B(8450); TMEM32
(93380); ARHGEF6(9459); HCFC1(3054); PRKY(5616); CYorf15A(246126);

Table 4-111

| | | |
|---|---|---|
| hsa-miR-571 | 2076; | TNFRSF1B(7133); NBL1(4681); EPB41(2035); LCK(3932); RBBP4(5928); |
| | | MACF1(23499); SH3GLB1(51100); RAP1A(5906); BCL9(607); BNIPL(149428); |
| | | FASLG(356); C1orf76(148753); NAV1(89796); ADORA1(134); IL19 |
| | | (29949); TNFRSF14(8764); FRAP1(2475); FAM77C(79570); RIMS3(9783); |
| | | MUTYH(4595); C1orf180(439927); C1orf183(55924);PYGO2(90780); KCNJ10 |
| | | (3766); DEDD(9191); KIF21B(23046); RHOB(388); PRKCE(5581); ARHGAP25 |
| | | (9938); HK2(3099); MRPL35(51318); BCL2L11(10018); PROC |
| | | (5624); TAIP-2(80034); FZD7(8324); ALS2CR19(117583); NRP2(8828); STK36 |
| | | (27148); SEPT2(4735); CGREF1(10669); PPM1G(5496); ADD2(119); |
| | | PAX8(7849); PCAF(8850); PDCD6IP(10015); CTDSPL(10217); CTNNB1 |
| | | (1499); RBM5(10181); PCNP(57092); ZDHHC23(254887); NEK11(79858); |
| | | ACPP(55); RNF7(9616); RTP1(132112); CX3CR1(1524); KIAA1143(57456); |
| | | SLC6A20(54716); RASSF1(11186); CISH(1154); FAM107A(11170); FHIT |
| | | (2272); EIF4E3(317649); CD47(961); GSK3B(2932); TFDP2(7029); CD38 |
| | | (952); LOC401137(401137); EREG(2069); FGF5(2250); LRPAP1(4043); |
| | | UNC5C(8633); LEF1(51176); SETD7(80854); ANAPC10(10393); NIPBL |
| | | (25836); CCNB1(891); APC(324); TCF7(6932); PCDHB9(56127); GRIA1(2890); |
| | | KCNIP1(30820); PRLR(5618); PRKAA1(5562); FGF10(2255); MCC |
| | | (4163); ATG12(9140); SEMA6A(57556); PPP2CA(5515); NME5(8382); CDC25C |
| | | (995); NRG2(9542); SLIT3(6586); DBN1(1627); E2F3(1871); GMNN |
| | | (51053); CSNK2B(1460); FOXP4(116113); CDC5L(988); IL17(3605); FOXO3A |
| | | (2309); EXOC2(55770); DHX16(8449); MOCS1(4337); CCND3(896); |
| | | TNFRSF21(27242); PKHD1(5314); DST(667); SGK(6446); LATS1(9113); |
| | | FZD1(8321); STAG3(10734); BCAP29(55973); ST7(7982); CALU(813); KIAA0265 |
| | | (23008); CASP2(835); MGC9712(202915); INHBA(3624); HIP1(3092); |
| | | PTN(5764); FASTK(10922); SPFH2(11160); BAG4(9530); TERF1(7013); |
| | | STMN2(11075); EFCBP1(64168); MTUS1(57509); LZTS1(11178); TNFRSF10B |
| | | (8795); ADRA1A(148); TRIM35(23087); CLU(1191); NPAL2(79815); |
| | | RAD21(5885); EXT1(2131); FBXO32(114907); UNC13B(10497); MAK10 |
| | | (60560); DAPK1(1612); PALM2(114299); CDK9(1025); IFNW1(3467); |
| | | CDKN2B(1030); GAS1(2619); GRIN3A(116443); TNFSF8(944); TRAF1(7185); |
| | | CIZ1(25792); UNC5B(219699); SFXN3(81855); BTRC(8945); NOLC1 |
| | | (9221); SUFU(51684); NEURL(9148); GPR26(2849); ZWINT(11130); DLG5 |
| | | (9231); SFTPA2(6436); GRID1(2894); FBXW4(6468); FGF8(2253); C10orf46 |
| | | (143384); STIM1(6786); GAS2(2620); EIF4G2(1982); KCNJ11(3767); |
| | | CLCF1(23529); ALDH3B2(222); FGF4(2249); LRRC32(2615); PAK1(5058); |
| | | SESN3(143686); ICEBERG(59082); TIMM8B(26521); IL18(3606); BCL9L |
| | | (283149); CCND2(894); CNAP1(9918); CDK2(1017); FAM62A(23344); |
| | | RBMS2(5939); MSRB3(253827); MDM2(4193); CUTL2(23316); OAS2(4939); |
| | | TCF1(6927); FZD10(11211); CACNA2D4(93589); NOL1(4839); FAIM2 |
| | | (23017); C12orf22(81566); MMP19(4327); BRAP(8315); SBNO1(55206); |
| | | ATP8A2(51761); SUGT1(10910); TFDP1(7027); LATS2(26524); CSNK1A1L |
| | | (122011); FOXO1A(2308); PCK2(5106); SLC7A8(23428); LTBP2(4053); |
| | | SPTLC2(9517); BAG5(9529); PPP1R13B(23368); MAP1A(4130); PML(5371); |
| | | NDN(4692); TP53BP1(7158); TBC1D2B(23102); TBL3(10607); TNFRSF12A |
| | | (51330); MMPL1(4328); PLK1(5347); PRRT2(112476); CYLD(1540); |
| | | PARD6A(50855); CIAPIN1(57019); SMPD3(55512); TERF2(7014); ALOX12 |
| | | (239); ALOX15B(247); MAPK7(5598); NF1(4763); LIG3(3980); THRA |
| | | (7067); FZD2(2535); DBF4B(80174); TOM1L1(10040); PRR11(55771); CARD14 |
| | | (79092); MNT(4335); P2RX1(5023); AURKB(9212); SPAG5(10615); |
| | | GAS2L2(246176); PLXDC1(57125); BRCA1(672); HDAC5(10014); PHB(5245); |
| | | MPO(4353); SLC39A11(201266); DTNA(1837); KIAA1328(57536); RNF165 |
| | | (494470); DCC(1630); PHLPP(23239); TXNL1(9352); BCL2(596); |
| | | GIPC3(126326); UHRF1(29128); DNM2(1785); MGC3207(84245); F2RL3 |
| | | (9002); PIK3R2(5296); APLP1(333); MAP3K10(4294); CYP2S1(29785); BCAM |
| | | (4059); APOE(348); BCL2L12(83596); PRMT1(3276); ATF5(22809): |
| | | PRKCG(5582); FLJ38288(284309); LOC113386(113386); TNFSF14(8740); |
| | | CDC37(11140); HSPB6(126393); CARD8(22900); A1BG(1); C20orf98(80023); |
| | | CBFA2T2(9139); CEP250(11190); EPB41L1(2036); CSNK2A1(1457); |
| | | SCRT2(85508); E2F1(1869); SON(6651); ITGB2(3689); NF2(4771); MCM5 |
| | | (4174)EP300(2033); ADM2(79924); MAPK8IP2(23542); CECR1(51816); |
| | | TOB2(10766); POLR3H(171568); MAPK12(6300); MSL3L1(10943); ZXDB |
| | | (158586); MLLT7(4303); NLGN3(54413); RP6-213H19.1(51765); CRSP2 |
| | | (9282); SYP(6855); HUWE1(10075); CUL4B(8450); CD99L2(83692); HCFC1 |
| | | (3054); |

Table 4-112

| | | |
|---|---|---|
| hsa-miR-579 | 2077; | CAMTA1(23261); CDC42(998); PDIK1L(149420); RBBP4(5928); UTP11L |
| | | (51118); MACF1(23499); PRKAA2(5563); GADD45A(1647); SH3GLB1(51100); |
| | | CDC7(8317); TMEM56(148534); CDC14A(8556); HIPK1(204851); SYCP1 |
| | | (6847); CKS1B(1163); FASLG(356); FAM20B(9917); NEK7(140609); |
| | | MDM4(4194); TRAF5(7188); CIAS1(114548); CDC2L2(985); TNFRSF9 |
| | | (3604); FRAP1(2475); RCC2(55920); CLSPN(63967); FAF1(11124); SASS6 |
| | | (163786); NRAS(4893); NOTCH2(4853); PYGO2(90780); ASPM(259266); |
| | | IL10(3586); LPGAT1(9926); USH2A(7399); C1orf96(126731); FH(2271); CHML |
| | | (1122); AHCTF1(25909); PPP1CB(5500); BIRC6(57448); PRKCE(5581); |
| | | MSH2(4436); PAPOLG(64895); GKN1(56287); HTRA2(27429); TCF7L1 |
| | | (83439); FLJ13910(64795); BRRN1(23397); LOC339745(339745); RIF1 |
| | | (55183); TAIP-2(80034); ZAK(51776); ITGA4(3676); PMS1(5378); CFLAR |
| | | (8837); CASP8(841); CD28(940); ALS2CR19(117583); CREB1(1385); COL4A3 |
| | | (1285); OACT2(129642); YWHAQ(10971); E2F6(1876); CARD12(58484); |
| | | EIF2AK2(5610); RTN4(57142); CCT4(10575); TIA1(7072); TGFA(7039); |
| | | EDAR(10913); SEPT10(151011); BUB1(699); ANAPC1(64682); IL1A |
| | | (3552); BIN1(274); CACNB4(785); IFIH1(64135); TLK1(9874); FRZB(2487); |
| | | NCKAP1(10787); STAT1(6772); STK17B(9262); ERBB4(2066); CUL3(8452); |
| | | HDAC4(9759); FANCD2(2177); PCAF(8850); PDCD6IP(10015); MLH1 |
| | | (4292); NKTR(4820); LIMD1(8994); IL17RB(55540); APPL(26060); 2′-PDE |
| | | (201626); PCNP(57092); PVRL3(25945); NEK11(79858); ACPP(55); |
| | | SMC4L1(10051); FXR1(8087); SENP2(59343); TP73L(8626); OPA1(4976); |
| | | NEK4(6787); UBE1C(9039); GSK3B(2932); TMCC1(23023); STAG1(10274); |
| | | PIK3CB(5291); TFDP2(7029); ATR(545); CD38(952); HCAP-G(64151); |
| | | ANAPC4(29945); MOBKL1A(92597); SLC4A4(8671); EREG(2069); DKFZP564O0823 |
| | | (25849); SEPT11(55752); FGF5(2250); AFF1(4299); SPP1(6696); |
| | | NFKB1(4790); PLK4(10733); TLR2(7097); APBB2(323); ATP8A1(10396); |
| | | BTC(685); UNC5C(8633); DNAJB14(79982); DKK2(27123); ANKRD50 |
| | | (57182); CTSO(1519); NEK1(4750); VEGFC(7424); CASP3(836); NIPBL |
| | | (25836); GZMA(3001); FLJ13611(80006); ERBB2IP(55914); CCNB1(891); |
| | | RAD17(5884); IQGAP2(10788); F2R(2149); CSPG2(1462); LRAP(64167); |
| | | APC(324); DCP2(167227); CSNK1G3(1456); LOC90624(90624); RAD50 |
| | | (10111); KCTD16(57528); GM2A(2760); SGCD(6444); BNIP1(662); PRLR |
| | | (5618); OSRF(23548); PRKAA1(5562); IL6ST(3572); IRF1(3659); AFF4 |
| | | (27125); CDC25C(995); FGF1(2246); CSNK1A1(1452); GABRB2(2561); FBXW11 |
| | | (23291); DDX41(51428); E2F3(1871); CSNK2B(1460); VEGF(7422); |
| | | CDC5L(988); IL17(3605); PTP4A1(7803); TTK(7272); POU3F2(5454); FOXO3A |
| | | (2309); HECA(51696); SASH1(23328); QKI(9444); NEDD9(4739); MDC1 |
| | | (9656); SUPT3H(8464); TNFRSF21(27242); DST(667); CCNC(892); ATG5 |
| | | (9474); SESN1(27244); HBS1L(10767); BCLAF1(9774); KATNA1(11104); |
| | | LATS1(9113); PDCD2(5134); AHR(196); HDAC9(9734); ZNRF2(223082); |
| | | SEPT7(989); STK17A(9263); EGFR(1956); FZD1(8321); STAG3(10734); |
| | | RINT1(60561); HBP1(26959); BCAP29(55973); CUL1(8454); TRIAD3 |
| | | (54476); PMS2(5395); SOSTDC1(25928); CYCS(54205); CARD4(10392); INHBA |
| | | (3624); HIP1(3092); CDK6(1021); PPP1R3A(5506); BRAF(673); ESCO2 |
| | | (157570); FZD3(7976); UNC5D(137970); BAG4(9530); TACC1(6867); |
| | | UBE2V2(7336); SULF1(23213); TERF1(7013); RIPK2(8767); OXR1(55074); |
| | | PINX1(54984); TNFRSF10B(8795); TNFRSF10D(8793); DUSP4(1846); |
| | | SFRP1(6422); CA8(767); TP53INP1(94241); TNFRSF11B(4982); FBXO32 |
| | | (114907); FAM84B(157638); CDC37L1(55664); JAK2(3717); DENND4C |
| | | (55667); UNC13B(10497); RECK(8434); NTRK2(4915); DAPK1(1612); C9orf47 |
| | | (286223); CKS2(1164); TGFBR1(7046); TMEM38B(55151); PALM2 |
| | | (114299); IFNW1(3467); IFNA2(3440); CDKN2B(1030); TAF1L(138474); |
| | | SEMA4D(10507); TXNDC4(23071); CTNNAL1(8727); TNFSF15(9966); TNFSF8 |
| | | (944); ZMYND11(10771); C10orf9(219771); SIRT1(23411); UNC5B |
| | | (219699); PTEN(5728); FAS(355); PCGF5(84333); KIF11(3832); ENTPD7 |
| | | (57089); CSPG6(9126); TCF7L2(6934); TRUB1(142940); DOCK1(1793); |
| | | ADARB2(105); IL2RA(3559); PARD3(56288); CUL2(8453); PLEKHK1(219790); |
| | | PPP3CB(5532); ATAD1(84896); SMNDC1(10285); DCLRE1A(9937); |
| | | C10orf46(143384); TIAL1(7073); MKI67(4288); BNIP3(664); IPO7(10527); |
| | | WEE1(7465); HIPK3(10114); BIRC3(330); BIRC2(329); CUL5(8065); |
| | | ATM(472); UBE4A(9354); IGF2(3481); DKK3(27122); PTH(5741); FANCF |
| | | (2188); WT1(7490); CD59(966); MTL5(9633); PGM2L1(283209); RAB30(27314); |
| | | FZD4(8322); SESN3(143686); CASP1(834); PPP2R1B(5519); IL18 |
| | | (3606); ZW10(9183); CCND2(894); CNAP1(9918); CDKN1B(1027); IAPP(3 |

Table 4-113

| | | |
|---|---|---|
| | | 375); ETNK1(55500); TEGT(7009); ESPL1(9700); CDK2(1017); PA2G4(5036); |
| | | MDM2(4193); CCT2(10576); APAF1(317); GAS2L3(283431); KNTC1 |
| | | (9735); FZD10(11211); RAN(5901); LRP6(4040); BCAT1(586); KRAS(3845); |
| | | ABCD2(225); SLC38A1(81539); C12orf22(81566); DDIT3(1649); PAWR(5074); |
| | | DUSP6(1848); WDR51B(282809); PCTK2(5128); DIP13B(55198); PPP1CC |
| | | (5501); DIABLO(56616); MPHOSPH9(10198); ZNF605(90462); HSMPP8 |
| | | (54737); FGF9(2254); BRCA2(675); RB1(5925); RFP2(10206); FLJ40296 |
| | | (122183); NDFIP2(54602); ING1(3621); CDC16(8881); LATS2(26524); |
| | | UBL3(5412); NEK3(4752); KLF12(11278); PNN(5411); CDKN3(1033); |
| | | MNAT1(4331); CHURC1(91612); PSEN1(5663); VASH1(22846); GPR65 |
| | | (8477); CALM1(801); ACIN1(22985); CIDEB(27141); STRN3(29966); EGLN3 |
| | | (112399); MAMDC1(161357); CDKL1(8814); DLG7(9787); LTBP2(4053); |
| | | DIO2(1734); CHES1(1112); MOAP1(64112); BAG5(9529); PPP1R13B |
| | | (23368); AKT1(207); BUB1B(701); MAPK6(5597); Gcom1(145781); CCNB2 |
| | | (9133); RAB11A(8766); FEM1B(10116); KIF23(9493); NDN(4692); SLC12A6 |
| | | (9990); BMF(90427); MYEF2(50804); PYGO1(26108); PRTG(283659); |
| | | BNIP2(663); CSNK1G1(53944); KREMEN2(79412); DNASE1(1773); DNAJA3 |
| | | (9093); CYLD(1540); RBL2(5934); NOL3(8996); CTCF(10664); AXIN1 |
| | | (8312); USP7(7874); GSPT1(2935); APPBP1(8883); HIC1(3090); PAFAH1B1 |
| | | (5048); NLK(51701); TRAF4(9618); CPD(1362); RAB11FIP4(84440); |
| | | CCL2(6347); LIG3(3980); CROP(51747); PPM1D(8493); TLK2(11011);PRKCA |
| | | (5578); KPNA2(3838); MAP2K6(5608); NALP1(22861); FLJ36492 |
| | | (284047); BECN1(8678); BRCA1(672); PHB(5245); PTRH2(51651); ERN1 |
| | | (2081); ARHGAP28(79822); CABLES1(91768); DCC(1630); MALT1(10892); |
| | | TNFRSF11A(8792); C18orf19(125228); MC2R(4158); ROCK1(6063); ESCO1 |
| | | (114799); PARD6G(84552); CSNK1G2(1455); CHAF1A(10036); UHRF1 |
| | | (29128); APOE(348); EMR2(30817); KIAA1559(57677); ZFP30(22835); |
| | | PLCB1(23236); CEP250(11190); STK4(6789); CSE1L(1434); PARD6B(84612); |
| | | PCNA(5111); RBL1(5933); UBE2V1(7335); AURKA(6790); CTCFL(140690); |
| | | SYCP2(10388); DIDO1(11083); GABPA(2551); USP16(10600); SON |
| | | (6651); SLC5A3(6526); MX1(4599); TMEM1(7109); BTG3(10950); YWHAH |
| | | (7533); PGEA1(25776); SEPT3(55964); NUP50(10762); GTSE1(51512); |
| | | BID(637); MAPK1(5594); MN1(4330); CHEK2(11200); DMC1(11144); SMC1L2 |
| | | (27127); HDAC6(10013); MAGEH1(28986); YIPF6(286451); MLLT7 |
| | | (4303); TAF1(6872); OGT(8473); GLT28D1(55849); BIRC4(331); STAG2 |
| | | (10735); RP6-213H19.1(51765); AFF2(2334); DKC1(1736); BRCC3(79184); |
| | | KAL1(3730); GPM6B(2824); RPS6KA3(6197); SMC1L1(8243); CHM(1121); |
| | | RP13-383K5.1(55285); SEPT6(23157); CUL4B(8450); ARHGEF6(9459); |
| | | CETN2(1069); MTCP1(4515); CLIC2(1193); |
| hsa-miR-584 | 2078; | CDC42(998); HDAC1(3065); UTP11L(51118); CDKN2C(1031); RNF11(26994); |
| | | GADD45A(1647); PKN2(5586); SLC30A7(148867); BCL9(607); UHMK1 |
| | | (127933); XPR1(9213); TGFB2(7042); ERRFI1(54206); SDC3(9672); |
| | | COL8A2(1296); TESK2(10420); PPAP2B(8613); NEGR1(257194); SSX2IP |
| | | (117178); GSTM3(2947); ENAH(55740); PPP1CB(5500); ZAK(51776); ITGA4 |
| | | (3676); FZD7(8324); COL4A3(1285); E2F6(1876); EIF2AK2(5610); MCFD2 |
| | | (90411); PLA2R1(22925); SCN1A(6323); KIAA1715(80856); STAT1 |
| | | (6772); CLK1(1195); FLJ23861(151050); HDAC4(9759); GALNTL2(117248); |
| | | PCAF(8850); NR1D2(9975); APPL(26060); MITF(4286); TP73L(8626); |
| | | OPA1(4976); CGGBP1(8545); CD38(952); PI4K2B(55300); SEPT11(55752); |
| | | C4orf12(404201); AFF1(4299); NFKB1(4790); WWC2(80014); GABRA4 |
| | | (2557); PRKG2(5593); FSTL5(56884); NIPBL(25836); APC(324); CSNK1G3 |
| | | (1456); FLJ37562(134553); SGCD(6444); PRLR(5618); PRKAA1(5562); |
| | | BIRC1(4671); MTX3(345778); SEMA6A(57556); AFF4(27125); PPP2CA |
| | | (5515); FBXW11(23291); ZNF192(7745); APOBEC2(10930); PTP4A1(7803); |
| | | FOXO3A(2309); SASH1(23328); ULBP1(80329); NEDD9(4739); ICK(22858); |
| | | DST(667); CCNC(892); ATG5(9474); SEC63(11231); STX7(8417); HBS1L |
| | | (10767); FKBP9(11328); FLJ10099(55069); GATAD1(57798); STAG3 |
| | | (10734); RINT1(60561); MDFIC(29969); PMS2(5395); EIF2AK1(27102); FKBP9L |
| | | (360132); CACNA2D1(781); MCFP(55972); CDK6(1021); SRPK2 |
| | | (6733); TNFRSF10C(8794); E2F5(1875); WWP1(11059); CNOT7(29883); CLU |
| | | (1191); WHSC1L1(54904); CALB1(793); RUNX1T1(862); SPIN(10927); |
| | | INVS(27130); FCMD(2218); BAG1(573); ARID5B(84159); PTEN(5728); |
| | | KIF11(3832); BTRC(8945); PDCD11(22984); ACADSB(36); DOCK1(1793); |
| | | PRKCQ(5588); CUL2(8453); PPP3CB(5532); LOC399818(399818); HIPK3 |
| | | (10114); MGC34830(120196); BIRC3(330); CUL5(8065); STS-1(84959) |

Table 4-114

| | | |
|---|---|---|
| | | ; TBRG1(84897); EIF4G2(1982); WT1(7490); CLCF1(23529); CHORDC1 |
| | | (26973); SESN3(143686); C12orf5(57103); CNAP1(9918); ETNK1(55500); |
| | | NEUROD4(58158); MDM2(4193); TMTC3(160418); CRADD(8738); GAS2L3 |
| | | (283431); CUTL2(23316); ZNF84(7637); MFAP5(8076); LRP6(4040); |
| | | KRAS(3845); BTG1(694); HSMPP8(54737); BRCA2(675); RB1(5925); CSNK1A1L |
| | | (122011); NEK3(4752); LIG4(3981); PNN(5411); PSEN1(5663); ENTPD5 |
| | | (957); LTBP2(4053); CHES1(1112); MTAC2D1(123036); PPP1R13B |
| | | (23368); 76P(27229); FGF7(2252); RNF111(54778); KIF23(9493); ZFP106 |
| | | (64397); TP53BP1(7158); CSNK1G1(53944); BCL2A1(597); FTS(64400); |
| | | SMPD3(55512); MIS12(79003); NF1(4763); TADA2L(6871); CROP(51747); |
| | | TLK2(11011); MAP2K6(5608); P2RX1(5023); BRCA1(672); LOC641522 |
| | | (641522); ROCK1(6093); ESCO1(114799); ZNF24(7572); TXNL1(9352); |
| | | CHAF1A(10036); ZNF493(284443); ZNF226(7769); ZNF583(147949); ZNF443 |
| | | (10224); CYP4F11(57834); ZNF585A(199704); SIRT2(22933); CARD8 |
| | | (22900); ZNF432(9668); ZNF677(342925); PLCB1(23236); PARD6B(84612); |
| | | UBE2V1(7335); USP16(10600); SON(6651); MORC3(23515); BTG3(10950); |
| | | DSCR3(10311); BRWD1(54014); EP300(2033); BID(637); MAPK1 |
| | | (5594); PDHA1(5160); BIRC4(331); STAG2(10735); RP6-213H19.1(51765); |
| | | RP11-393H10.2(256714); SMC1L1(8243); ZDHHC15(158866); MECP2 |
| | | (4204); |
| hsa-miR-589 | 2079; | CDC42(998); SESN2(83667); RBBP4(5928); MACF1(23499); NFYC(4802); |
| | | GADD45A(1647); CDC14A(8556); UHMK1(127933); DDR2(4921); ADORA1 |
| | | (134); MDM4(4194); TGFB2(7042); CIAS1(114548); TNFRSF14(8764); |
| | | RERE(473); CTNNBIP1(56998); FRAP1(2475); RCC2(55920); FUSIP1 |
| | | (10772); KIAA0319L(79932); MUTYH(4595); TESK2(10420); FAF1(11124); |
| | | MCL1(4170); PYGO2(90780); FCRL5(83416); DEDD(9191); ASPM(259266); |
| | | TOMM20(9804); BIRC6(57448); CCT7(10574); HTRA2(27429); TCF7L1 |
| | | (83439); MAL(4118); ACTR3(10096); PROC(5624); TAIP-2(80034); ZAK |
| | | (51776); CASP10(843); INHA(3623); CARD12(58484); CYP26B1(56603); |
| | | REEP1(65055); SEPT10(151011); ANAPC1(64682); FLJ10996(54520); |
| | | MCM6(4175); IFIH1(64135); RAPH1(65059); TNS1(7145); CUL3(8452); TRIP12 |
| | | (9320); FANCD2(2177); PCAF(8850); MOBP(4336); CTNNB1(1499); |
| | | CCR9(10803); FXR1(8087); PSMD2(5708); ADIPOQ(9370); RAF1(5894); |
| | | ZCSL2(285381); ZNF445(353274); PRICKLE2(166336); TMCC1(23023); |
| | | STAG1(10274); TFDP2(7029); ATR(545); SIAH2(6478); TNFSF10(8743); |
| | | WHSC1(7468); HCAP-G(64151); SEPT11(55752); CCNG2(901); FGF2(2247); |
| | | GAK(2580); PPBP(5473); UNC5C(8633); LEF1(51176); MAD2L1(4085); |
| | | VEGFC(7424); SKP2(6502); ERBB2IP(55914); CCNB1(891); RAD17(5884); |
| | | F2R(2149); APC(324); TCF7(6932); SEMA5A(9037); PRLR(5618); PRKAA1 |
| | | (5562); MTX3(345778); MCC(4163); CDC23(8697); HDAC3(8841); |
| | | FGF1(2246); FBXW11(23291); DDX41(51428); CDKN1A(1026); SLC25A27 |
| | | (9481); TTK(7272); TPBG(7162); C6orf68(116150); TNFAIP3(7128); HECA |
| | | (51696); NEDD9(4739); IER3(8870); DAXX(1616); TNFRSF21(27242); |
| | | MCM3(4172); DST(667); TSPYL1(7259); SGK(6446); PLAGL1(5325); PDCD2 |
| | | (5134); HDAC9(9734); SEPT7(989); FZD9(8326); STAG3(10734); HBP1 |
| | | (26959); CASP2(835); TRIAD3(54476); PMS2(5395); INHBA(3624); YWHAG |
| | | (7532); SEMA3E(9723); CDK6(1021); UBE2H(7828); ZC3HC1(51530); |
| | | BRAF(673); ESCO2(157570); FZD3(7976); TACC1(6867); SULF1(23213); |
| | | WISP1(8840); TSPYL5(85453); STK3(6788); RAD21(5885); MTSS1 |
| | | (9788); ANKRD15(23189); CDC37L1(55664); JAK2(3717); UNC13B(10497); |
| | | RECK(8434); NTRK2(4915); DAPK1(1612); C9orf47(286223); FSD1CL |
| | | (405752); CDK9(1025); ABL1(25); FBXVW2(26190); TSC1(7248); CARD9(64170); |
| | | CUGBP2(10659); DKK1(22943); PTEN(5728); SUFU(51684); CSPG6 |
| | | (9126); BAG3(9531); PARD3(56288); C10orf72(196740); PPP3CB(5532); |
| | | CPEB3(22849); MKI67(4288); BNIP3(664); STIM1(6786); WEE1(7465); |
| | | GAS2(2620); HIPK3(10114); GPIAP1(4076); API5(8539); EXT2(2132); F2 |
| | | (2147); DPF2(5977); LRP5(4041); CCND1(595); BIRC3(330); CUL5(8065); |
| | | ATM(472); EI24(9538); APBB1(322); DKK3(27122); NXF1(10482); PAK1 |
| | | (5058); FZD4(8322); SESN3(143686); CASP4(837); CASP5(838); PPP2R1B |
| | | (5519); CCND2(894); LTBR(4055); TEGT(7009); MDM2(4193); CCT2(10576); |
| | | RFX4(5992); KNTC1(9735); LRP6(4040); FAIM2(23017); CBX5(23468); |
| | | CS(1431); GIT2(9815); ANAPC7(51434); ANAPC5(51433); MPHOSPH9 |
| | | (10198); SCARB1(949); GOLGA3(2802); FGF9(2254); BRCA2(675); RB1 |
| | | (5925); RFP2(10206); HS6ST3(266722); ARHGEF7(8874); CDC16(8881); |
| | | KLF12(11278); CDKN3(1033); CGRRF1(10668); SLC39A9(55334); PSEN |

Table 4-115

| | | |
|---|---|---|
| | | 1(5663); TRAF3(7187); STRN3(29966); MAP3K9(4293); LTBP2(4053); CHES1 |
| | | (1112); C14orf103(55102); BAG5(9529); GOLGA8E(390535); GOLGA8G |
| | | (283768); KIAA1018(22909); 76P(27229); MAPK6(5597); KIF23(9493); |
| | | SCAMP5(192683); NIPA1(123606); SLC12A6(9990); BMF(90427); PYGO1 |
| | | (26108); BNIP2(663); CSNK1G1(53944); TSC2(7249); CYLD(1540); |
| | | RBL2(5934); CTCF(10664); SMG1(23049); ZNF694(342357); PAFAH1B1 |
| | | (5048); GSG2(83903); NF1(4763); NMT1(4836); MAPT(4137); NGFR(4804); |
| | | CROP(51747); WDR68(10238); MAP2K6(5608); SPHK1(8877); GAS7(8522); |
| | | SPAG5(10615); AP1GBP1(11276); BECN1(8678); BRCA1(672); ARHGAP27 |
| | | (201176); ZNF652(22834); MPO(4353); ERN1(2081); ACOX1(51); |
| | | MXRA7(439921); CIDEA(1149); DTNA(1837); SMAD4(4089); DCC(1630); |
| | | PHLPP(23239); BCL2(596); MBP(4155); CDC34(997); TNFSF9(8744); CCNE1 |
| | | (898); PSMD8(5714); AXL(558); BCL2L12(83596); ZNF585A(199704); |
| | | DPF1(8193); TRIB3(57761); MCM8(84515); CEP250(11190); CSE1L |
| | | (1434); PARD6B(84612); BIRC7(79444); RBL1(5933); PTPRT(11122); CTCFL |
| | | (140690); DIDO1(11083); SON(6651); SLC5A3(6526); APP(351); BRWD1 |
| | | (54014); SNF1LK(150094); BCL2L13(23786); GAS2L1(10634); NF2(4771); |
| | | YWHAH(7533); TNRC6B(23112); EP300(2033); BID(637); MAPK1 |
| | | (5594); PPM1F(9647); CARD10(29775); NHP2L1(4809); AGTR2(186); BIRC4 |
| | | (331); DKC1(1736); SEPT6(23157); CUL4B(8450); PDCD8(9131); ARHGEF6 |
| | | (9459); MTCP1(4515); |
| hsa-miR-591 | 2080; | TP73(7161); UBXD3(127733); TMEM50A(23585); RCC1(1104); RBBP4(5928); |
| | | MACF1(23499); PTGFR(5737); PROK1(84432); PPP1R12B(4660); ADORA1 |
| | | (134); SMYD2(56950); DFFA(1676); ZBTB17(7709); E2F2(1870); |
| | | FABP3(2170); TESK2(10420); SSX2IP(117178); C1orf183(55924); NOTCH2 |
| | | (4853); ASTN(460); HTRA2(27429); TCF7L1(83439); BCL2L11(10018); |
| | | PROC(5624); PMS1(5378); ALS2CR19(117583); COL4A3(1285); BOK(666); |
| | | PPM1G(5496); CARD12(58484); MCFD2(90411); EDAR(10913); ANAPC1 |
| | | (64682); CLASP1(23332); HS6ST1(9394); IFIH1(64135); FRZB(2487); |
| | | CLK1(1195); HDAC4(9759); CD200(4345); MCM2(4171); FXR1(8087); OPA1 |
| | | (4976); CIDEC(63924); RAF1(5894); CCDC71(64925); RAB43(339122); |
| | | PIK3CB(5291); ATR(545); HD(3064); CD38(952); LEF1(51176); CASP3 |
| | | (836); PDCD6(10016); FAM105B(90268); GZMA(3001); APC(324); TCF7 |
| | | (6932); BIRC1(4671); CDC23(8697); DUSP1(1843); RREB1(6239); CSNK2B |
| | | (1460); MAPK13(5603); C6orf71(389434); ZDHHC14(79683); DAXX(1616); |
| | | MCM3(4172); DST(667); PGM3(5238); BCLAF1(9774); THBS2(7058); |
| | | PDCD2(5134); HDAC9(9734); SEPT7(989); EGFR(1956); FZD9(8326); DMTF1 |
| | | (9988); ADAM22(53616); STAG3(10734); CASP2(835); ETV1(2115); |
| | | CYCS(54205); LOC442535(442535); NUDCD3(23386); FZD3(7976); RIPK2 |
| | | (8767); ADRA1A(148); TRIM35(23087); PAG1(55824); RAD21(5885); JRK |
| | | (8629); MTAP(4507); NTRK2(4915); C9orf47(286223); TGFBR1(7046); |
| | | FANCC(2176); PTCH(5727); ZNF510(22869); AKNA(80709); PPP6C(5537); |
| | | AK1(203); INPP5E(56623); ANAPC2(29882); C10orf104(119504); PDCD11 |
| | | (22984); C10orf39(282978); PPP3CB(5532); DLG5(9231); DCLRE1A |
| | | (9937); MKI67(4288); GAS2(2620); EHF(26298); PPP2R5B(5526); LOH11 |
| | | CR2A(4013); IGF2(3481); SBF2(81846); FZD4(8322); BTBD15(29068); CNAP1 |
| | | (9918); ESPL1(9700); PDE1B(5153); KRAS(3845); FMNL3(91010); |
| | | C12orf22(81566); CS(1431); RFP2(10206); ING1(3621); CUL4A(8451); CDC16 |
| | | (8881); NEK3(4752); GPR65(8477); OTUB2(78990); C14orf132(56967); |
| | | TRAF3(7187); LTBP2(4053); NEK9(91754); ALKBH1(8846); PPP1R13B |
| | | (23368); CGNL1(84952); C15orf42(90381); ARRDC4(91947); TSPAN3 |
| | | (10099); KIAA1924(197335); TSC2(7249); CCN7(899); CYLD(1540); PKMYT1 |
| | | (9088); RRN3(54700); BOLA2(552900); MAPK3(5595); SLC12A4(6560); |
| | | ALOX12(239); TNFSF12(8742); NF1(4763); MLLT6(4302); MAPT(4137); |
| | | CROP(51747); PPM1E(22843); PRKCA(5578); CARD14(79092); MNT |
| | | (4335); P2RX1(5023); TIAF1(9220); GAS2L2(246176); MPO(4353); SOCS3 |
| | | (9021); DTNA(1837); MAPK4(5596); MALT1(10892); PHLPP(23239); SERPINB2 |
| | | (5055); LMAN1(3998); TXNDC10(54495); TXNL4A(10907); EDG6 |
| | | (8698); CHAF1A(10036); UBA52(7311); KLK2(3817); PPP2R1A(5518); ZNF264 |
| | | (9422); DNASE2(1777); ERCC2(2068); CARD8(22900); CTNNBL1 |
| | | (56259); STK4(6789); C20orf112(140688); CABLES2(81928); DIDO1(11083); |
| | | NF2(4771); MCM5(4174); EP300(2033); SEPT3(55964); GAB4(128954); |
| | | MN1(4330); CARD10(29775); NPTXR(23467); SMC1L1(8243); SPIN2 |
| | | (54466); SEPT6(23157); TSPY1(7258); |

Table 4-116

| | | |
|---|---|---|
| hsa-miR-593 | 2081; | TP73(7161); KCNAB2(8514); CAMTA1(23261); H6PD(9563); SPSB1(80176); |
| | | PAX7(5081); RCC1(1104); SERINC2(347735); LCK(3932); GJA4(2701); |
| | | NCDN(23154); MACF1(23499); CDC20(991); PLK3(1263); C1orf83(127428); |
| | | ACOT11(26027); GNAI3(2773); FAM46C(54855); RBM8A(9939); BCL9 |
| | | (607); IGSF4B(57863); FLJ35530(400798); PKP1(5317); IPO(55705); |
| | | PPP1R12B(4660); ADORA1(134); IL24(11009); C1orf95(375057); RHOU |
| | | (58480); EDARADD(128178); MTR(4548); CDC2L2(985); RPL22(6146); |
| | | CASP9(842); ZBTB17(7709); ALDH4A1(8659); E2F2(1870); FUSIP1(10772); |
| | | YARS(8565); SIKE(80143); IGSF3(3321); NOTCH2(4853); MCL1(4170); |
| | | PYGO2(90780); NMNAT2(23057); TP53BP2(7159); TMEM63A(9725); |
| | | ZNF496(84838); DPYSL5(56896); TRIM54(57159); LYCAT(253558); BIRC6 |
| | | (57448); PRKCE(5581); HTRA2(27429); HK2(3099); MAL(4118); BRRN1 |
| | | (23397); BCL2L11(10018); ARHGEF4(50649); FMNL2(114793); TAIP- |
| | | 2(80034); ZAK(51776); PMS1(5378); CFLAR(8837); CASP10(843); CASP8 |
| | | (841); ALS2CR19(117583); TUBA4(80086); SEPT2(4735); PPM1G(5496); |
| | | CARD12(58484); RTN4(57142); TGFA(7039); SFXN5(94097); EDAR(10913); |
| | | BUB1(699); BIN1(274); ERCC3(2071); GTDC1(79712); NCKAP1(10787); |
| | | PAX3(5077); C2orf19(394261); FANCD2(2177); MKRN2(23609); NR2C2 |
| | | (7182); PDCD6IP(10015); DLEC1(9940); CTNNB1(1499); TREX1(11277); |
| | | SPCS1(28972); APPL(26060); ROBO2(6092); NEK11(79858); MRAS |
| | | (22808); KLHL24(54800); SENP2(59343); SRGAP3(9901); TADA3L(10474); |
| | | TMEM16K(55129); LTF(4057); TRAIP(10293); TUSC4(10641); CACNA2D2 |
| | | (9254); IL17RD(54756); PRICKLE2(166336); ADCY5(111); TFDP2 |
| | | (7029); LRRC15(131578); BDH1(622); PCGF3(10336); HD(3064); SEPT11 |
| | | (55752); NFKB1(4790); TMEM129(92305); MXD4(10608); LRPAP1(4043); |
| | | APBB2(323); UNC5C(8633); DNAJA5(134218); NIPBL(25836); CCNB1 |
| | | (891); APC(324); MFAP3(4238); UBXD8(23197); UNC5A(90249); N4BP3(23138); |
| | | TPPP(11076); PRLR(5618); RPL37(6167); P18SRP(285672); BIRC1 |
| | | (4671); SEMA6A(57556); PPP2CA(5515); CDC23(8697); APBB3(10307); |
| | | CAMK2A(815); CD74(972); ADAM19(8728); FBXW11(23291); TNF(7124); |
| | | PACSIN1(29993); PARC(23113); VEGF(7422); PTP4A1(7803); FOXO3A |
| | | (2309); TNFAIP3(7128); HECA(51696); MDC1(9656); MDGA1(266727); TRAM2 |
| | | (9697); DST(667); HS3ST5(222537); SGK(6446); LATS1(9113); PDCD2 |
| | | (5134); TTYH3(80727); FKBP9(11328); UBE2D4(51619); EGFR(1956); |
| | | FZD9(8326); ZFP95(23660); TRIAD3(54476); PMS2(5395); CARD4(10392); |
| | | URG4(55665); FKBP9L(360132); CDK6(1021); MCM7(4176); FIS1 |
| | | (51024); ZC3HC1(51530); HIPK2(28996); POLR3D(661); TNFRSF10C(8794); |
| | | BNIP3L(665); PTK2B(2185); UNC5D(137970); TACC1(6867); FAM77D |
| | | (286183); SULF1(23213); PI15(51050); CPNE3(8895); WISP1(8840); KIAA1875 |
| | | (340390); PSD3(23362); TNFRSF10B(8795); CLU(1191); KIF13B |
| | | (23303); FGFR1(2260); DKK4(27121); STK3(6788); JAK2(3717); UNC13B |
| | | (10497); DAPK1(1612); C9orf47(286223); TTC16(158248); ABL1(25); ADAMTS13 |
| | | (11093); RXRA(6256); CDKN2A(1029); TAF1L(138474); ZBTB5 |
| | | (9925); MCART1(92014); SEMA4D(10507); OGN(4969); PTCH(5727); TRIM14 |
| | | (9830); TRAF1(7185); CIZ1(25792); TSC1(7248); C9orf157(402381); |
| | | CARD9(64170); ANAPC2(29882); ZNF488(118738); EIF4EBP2(1979); SGPL1 |
| | | (8879); UNC5B(219699); BTRC(8945); SUFU(51684); PDCD11(22984); |
| | | CSPG6(9126); IL2RA(3559); C10orf72(196740); DNAJB12(54788); SFTPA2 |
| | | (6436); FBXW4(6468); C10orf46(143384); EPS8L2(64787); TEAD1 |
| | | (7003); DKFZp686O24166(374383); TMEM86A(144110); GAS2(2620); |
| | | HIPK3(10114); STX3A(6809); C11orf11(747); INCENP(3619); VEGFB(7423); |
| | | TPCN2(219931); CCND1(595); BIRC2(329); CUL5(8065); PHLDB1(23187); |
| | | ARHGEF12(23365); EI24(9538); NAP1L4(4676); RHOG(391); RAB6IP1 |
| | | (23258); DKK3(27122); MAP3K11(4296); FLJ90834(283135); ORAOV1 |
| | | (220064); GAB2(9846); GRM5(2915); JOSD3(79101); BCL9L(283149); |
| | | THY1(7070); OPCML(4978); CCND2(894); NTF3(4908); LTBR(4055); IAPP |
| | | (3375); DAZAP2(9802); ACVR1B(91); ESPL1(9700); SP1(6667); METTL7B |
| | | (196410); CCT2(10576); CRADD(8738); GAS2L3(283431); LNK(10019); |
| | | FGF6(2251); NOL1(4839); ING4(51147); LRP6(4040); BCAT1(586); PRICKLE1 |
| | | (144165); HDAC7A(51564); ADCY6(112); KIAA0286(23306); NFYB |
| | | (4801); GIT2(9815); MPHOSPH9(10198); SCARB1(949); CHFR(55743); |
| | | FGF9(2254); TNFRSF19(55504); BRCA2(675); RFP2(10206); LATS2(26524); |
| | | FOXO1A(2308); DKFZp451A211(400169); BCL2L2(599); TXNDC(81542); |
| | | VASH1(22846); KIAA0284(283638); KIAA0125(9834); LTBP2(4053); |
| | | NEK9(91754); CHES1(1112); C14orf143(90141); ANKRD9(122416); BA |

Table 4-117

| | | |
|---|---|---|
| | | G5(9529); PPP1R13B(23368); JAG2(3714); GANC(2595); RAB11A(8766); |
| | | CSK(1445); IL16(3603); ZSCAN2(54993); ZNF592(9640); ALPK3(57538); |
| | | SLC12A6(9990); SUHW4(54816); CSNK1G1(53944); PDCD7(10081); DPP8 |
| | | (54878); RGMA(56963); E4F1(1877); CCNF(899); ZNF213(7760); DNASE1 |
| | | (1773); GLIS2(84662); PLK1(5347); PSKH1(5681); CDT1(81620); C16orf28 |
| | | (65259); NME3(4832); FAM100A(124402); GSPT1(2935); THUMPD1 |
| | | (55623); ZNF694(342357); MAPK3(5595); C16orf58(64755); SIAH1(6477); |
| | | CMTM4(146223); SLC12A4(6560); SMPD3(55512); HSDL1(83693); PAFAH1B1 |
| | | (5048); ALOX12(239); SMCR7(125170); MAPK7(5598); TRAF4 |
| | | (9618); LIG3(3980); RASL10B(91608); SOCS7(30837); WIRE(147179); MAPT |
| | | (4137); PPM1D(8493); MAP2K6(5608); ARMC7(79637); KIAA0195(9772); |
| | | SPHK1(8877); BIRC5(332); CARD14(79092); C17orf27(57674); ANAPC11 |
| | | (51529); TBCD(6904); MNT(4335); P2RX1(5023); ATP2A3(489); NALP1 |
| | | (22861); GPS2(2874); TP53(7157); FLOT2(2319); MYO1D(4642); RAD51L3 |
| | | (5892); DDX52(11056); BRCA1(672); DUSP3(1845); MPP2(4355); |
| | | HDAC5(10014); ANKRD40(91369); MPO(4353); SCN4A(6329); PECAM1 |
| | | (5175); HELZ(9931); NT5C(30833); RNF157(114804); TMC6(11322); SOCS3 |
| | | (9021); C17orf55(284185); CIDEA(1149); PHLPP(23239); MBD1(4152); |
| | | FSTL3(10272); DOT1L(84444); SPPL2B(56928); FZR1(51343); PIK3R2 |
| | | (5296); GATAD2A(54815); APLP1(333); AXL(558); BCL3(602); APOE(348); |
| | | GRLF1(2909); BAX(581); ATF5(22809); LOC91661(91661); AURKC(6795); |
| | | ZNF551(90233); MBD3(53615); PLEKHJ1(55111); MATK(4145); TNFSF14 |
| | | (8740); BRD4(23476); DPF1(8193); SIRT2(22933); PRX(57716); DEDD2 |
| | | (162989); ERF(2077); ERCC2(2068); BBC3(27113); TRIB3(57761); CDC25B |
| | | (994); MCM8(84515); C20orf91(284800); KIF3B(9371); CEP250(11190); |
| | | MYBL2(4605); CD40(958); RTEL1(51750); RNF24(11237); BCL2L1 |
| | | (598); RIMS4(140730); ELMO2(63916); TMEPAI(56937); DIDO1(11083); |
| | | GM632(57473); USP16(10600); SON(6651); CHAF1B(8208); PRMT2(3275); |
| | | C21orf77(55264); SNF1LK(150094); ITGB2(3689); BCL2L13(23786); |
| | | DGCR8(54487); P2RXL1(9127); SMARCB1(6598); ADORA2A(135); GAS2L1 |
| | | (10634); NF2(4771); YWHAH(7533); KDELR3(11015); TEF(7008); SEPT3 |
| | | (55964); PPARA(5465); GTSE1(51512); DIP(23151); CECR6(27439); |
| | | HTF9C(27037); MAPK1(5594); PPM1F(9647); SUHW2(140883); ZNF70 |
| | | (7621); CHEK2(11200); DUSP18(150290); APOL4(80832); UNC84B(25777); |
| | | PDGFB(5155); TOB2(10766); MAPK12(6300); SHOX(6473); PCTK1(5127); |
| | | HDAC6(10013); CCNB3(85417); MSN(4478); TAF1(6872); NGFRAP1 |
| | | (27018); BIRC4(331); RP6-213H19.1(51765); OPHN1(4983); RNF12(51132); |
| | | PDCD8(9131); CD99L2(83692); |
| hsa-miR-595 | 2082; | ZBTB40(9923); SESN2(83667); TXLNA(200081); KIAA1522(57648); MACF1 |
| | | (23499); NFYC(4802); MGC33556(339541); AK5(26289); PROK1(84432); |
| | | CKS1B(1163); SCYL1BP1(92344); PRELP(5549); CIAS1(114548); TNFRSF9 |
| | | (3604); FRAP1(2475); FABP3(2170); FAF1(11124); BCL10(8915); COL24A1 |
| | | (255631); TGFBR3(7049); GFI1(2672); NOTCH2(4853); FCRL5 |
| | | (83416); DEDD(9191); SH2D1B(117157); RGS5(8490); STX6(10228); GLT25D2 |
| | | (23127); TNNI1(7135); TP53BP2(7159); C1orf96(126731); FLJ25143 |
| | | (130813); LPIN1(23175); RHOB(388); CAD(790); HK2(3099); TCF7L1(83439); |
| | | KCNIP3(30818); RIF1(55183); ZAK(51776); CASP8(841); CD28(940); |
| | | COL4A3(1285); LOC93349(93349); PSMD1(5707); YWHAQ(10971); CGREF1 |
| | | (10669); EIF2AK2(5610); FLJ31438(130162); SERTAD2(9792); ST6GAL2 |
| | | (84620); FLJ10996(54520); DARS(1615); TLK1(9874); NCKAP1 |
| | | (10787); FLJ43879(401039); HDAC4(9759); FANCD2(2177); CRTAP(10491); |
| | | PDCD6IP(10015); MOBP(4336); TIPARP(25976); LPP(4026); TP73L(8626); |
| | | SENP5(205564); OXTR(5021); TADA3L(10474); ZCSL2(285381); THRB |
| | | (7068); MAP4(4134); SFMBT1(51460); FHIT(2272); FOXP1(27086); |
| | | PROK2(60675); STAG1(10274); EIF5A2(56648); FGFRL1(53834); WHSC1 |
| | | (7468); LOC345222(345222); STK32B(55351); CPEB2(132864); EREG |
| | | (2069); SEPT11(55752); FRAS1(80144); AFF1(4299); PLK4(10733); TRIM2 |
| | | (23321); STOX2(56977); MLR1(254251); RNF150(57484); NR3C2(4306); |
| | | VEGFC(7424); CARD6(84674); ITGA2(3673); FLJ13611(80006); F2R(2149); |
| | | RASA1(5921); RHOBTB3(22836); TCF7(6932); GABRA1(2554); SEMA5A |
| | | (9037); DAP(1611); PRLR(5618); PPAP2A(8611); FLJ44796(401209); |
| | | AFF4(27125); HDAC3(8841); TNFAIP3(7128); PHACTR2(9749); SASH1 |
| | | (23328); DST(667); DJ122O8.2(57226); HBS1L(10767); LATS1(9113); TMEM106B |
| | | (54664); MPP6(51678); GHRHR(2692); EGFR(1956); ZNF138(7697); |
| | | PMS2L5(5383); HBP1(26959); TES(26136); MET(4233); CASP2(835 |

Table 4-118

| | | |
|---|---|---|
| | | ); MGC11257(84310); INHBA(3624); H2AFV(94239); PURB(5814); FLJ45974 |
| | | (401337); YWHAG(7532); CDK6(1021); ZNF3(7551); RABL5(64792); |
| | | GPR85(54329); ZC3HC1(51530); HIPK2(28996); KIAA0773(9715); CHMP7 |
| | | (91782); FZN3(7976); LEPROTL1(23484); UNC5D(137970); SPFH2(11160); |
| | | BAG4(9530); UBE2V2(7336); C8orf72(90362); SULF1(23213); ATP6V1C1 |
| | | (528); SLC30A8(169026); FAM91A1(157769); WISP1(8840); C8orf30A |
| | | (51236); SOX7(83595); CNOT7(29883); ENTPD4(9583); CLU(1191); WHSC1L1 |
| | | (54904); SFRP1(6422); ST18(9705); REXO1L1(254958); CCNE2 |
| | | (9134); HAS2(3037); FBXO32(114907); ANKRD15(23189); UNC13B(10497); |
| | | NTRK2(4915); SLC31A1(1317); OLFML2A(169611); TOR1B(27348); GPR107 |
| | | (57720); PRDM12(59335); SEMA4D(10507); DIRAS2(54769); AKNA |
| | | (80709); TNFSF15(9966); ZDHHC12(84885); QSCN6L1(169714); DKK1 |
| | | (22943); REEP3(221035); SIRT1(23411); UNC5B(219699); PCGF5(84333); |
| | | ENTPD7(57089); SFXN2(118980); SORCS3(22986); TCF7L2(6934); |
| | | PTPRE(5791); FBXW4(6468); MGEA5(10724); C10orf46(143384); IFITM1 |
| | | (8519); ZFP91(80829); FLJ10661(55199); CUL5(8065); ATM(472); NCAM1 |
| | | (4684); STS-1(84959); IGF2(3481); SLC22A8(9376); HBXAP(51773); RAB30 |
| | | (27314); FZD4(8322); PPP2R1B(5519); TSPAN9(10867); LTBR(4055); |
| | | TNFRSF7(939); PTMS(5763); CDKN1B(1027); DNM1L(10059); PA2G4 |
| | | (5036); APAF1(317); GAS2L3(283431); BRI3BP(140707); CACNA2D4(93589); |
| | | GPR92(57121); C1RL(51279); GRIN2B(2904); SOX5(6660); KIF21A |
| | | (55605); PAWR(5074); PCTK2(5128); DIP13B(55198); PPP1CC(5501); TNFRSF19 |
| | | (55504); ATP8A2(51761); ITR(160897); LATS2(26524); FOXO1A |
| | | (2308); FLJ10357(55701); ARHGAP5(394); MGAT2(4247); KIAA0247(9766); |
| | | VASH1(22846); NRXN3(9369); SLC24A4(123041); TCL6(27004); KIAA0329 |
| | | (9895); TRAF3(7187); GZMH(2999); STRN3(29966); PGF(5228); |
| | | NEK9(91754); SEL1L(6400); CHES1(1112); BAG5(9529); PPP1R138(23368); |
| | | SPRED1(161742); RNF111(54778); CCNB2(9133); FAM103A1(83640); |
| | | NDN(4692); SLC12A6(9990); MYEF2(50804); BNIP2(663); PDCD7(10081); |
| | | CCNF(899); PDPK1(5170); DNASE1(1773); ITGAL(3683); WWP2(11060); |
| | | MARVELD3(91862); RPL13(6137); ADCY9(115); MIR16(51573); CSNK2A2 |
| | | (1459); FANCA(2175); MGC3101(79007); ZNF286(57335); NLK(51701); |
| | | TRAF4(9618); NF1(4763); GOSR2(9570); NGFR(4804); CROP(51747); |
| | | TLK2(11011); PRKCA(5578); CARD14(79092); KIAA0753(9851); GAS7 |
| | | (8522); FAM106A(80039); SEPT4(5414); ERN1(2081); HELZ(9931); H3F3B |
| | | (3021); ACOX1(51); TBC1D16(125058); RNMT(8731); MAPRE2(10982); |
| | | YES1(7525); C18orf19(125228); DYM(54808); LMAN1(3998); BCL2(596); |
| | | GADD45B(4616); TNFSF9(8744); ILF3(3609); TPM4(7171); ZNF431 |
| | | (170959); DAPK3(1613); ZNF585A(199704); DEDD2(162989); DUXA(503835); |
| | | PLCB1(23236); SNRPB2(6629); CSE1L(1434); PHACTR3(116154); |
| | | CSNK2A1(1457); RSPO4(343637); RASSF2(9770); RBBP9(10741); BCL2L1 |
| | | (598); PLAGL2(5326); RBM12(10137); RBL1(5933); SYCP2(10388); CABLES2 |
| | | (81928); DIDO1(11083); CHAF1B(8208); CRYZL1(9946); PLAC4 |
| | | (191585); UBE2G2(7327); SLC19A1(6573); BCL2L13(23786); NF2(4771); |
| | | TNRC6B(23112); EP300(2033); BIK(638); PPARA(5465); PITPNB(23760); |
| | | PDGFB(5155); PRRG1(5638); OGT(8473); DIAPH2(1730); RP11-321G1.1 |
| | | (54885); AFF2(2334); KAL1(3730); ARHGAP6(395); SMC1L1(8243); RGAG4 |
| | | (340526); RP13-383K5.1(55285); HCFC1(3054); MECP2(4204); |
| hsa-miR-599 | 2083; 2129; | PRKCZ(5590); PIK3CD(5293); RBBP4(5928); MACF1(23499); CDC20(991); |
| | | NFIA(4774); CDC14A(8556); CHRNB2(1141); UHMK1(127933); C1orf21 |
| | | (81563); IL19(29949); RPS6KC1(26750); TGFB2(7042); C1orf128(200197); |
| | | RCC2(55920); E2F2(1870); CNR2(1269); SDC3(9672); CLSPN(63967); |
| | | WDR78(79819); GFI1(2672); MCL1(4170); C1orf32(387597); BIRC6(57448); |
| | | PRKCE(5581); CASP10(843); COL4A3(1285); FARP2(9855); CGREF1 |
| | | (10669); MCFD2(90411); TIA1(7072); ANAPC1(64682); MCM6(4175); IFIH1 |
| | | (64135); STAT1(6772); HDAC4(9759); FANCD2(2177); APPL(26060); |
| | | PCNP(57092); MCM2(4171); NEK11(79858); OPA1(4976); SLC6A20(54716); |
| | | STAG1(10274); EIF5A2(56648); HD(3064); SLC4A4(8671); MTTP(4547); |
| | | MYOZ2(51778); MAB21L2(10586); GABRG1(2565); SNCA(6622); SLC7A11 |
| | | (23657); MAML3(55534); KIAA1909(153478); NIPBL(25836); CARD6 |
| | | (84674); APC(324); CSNK1G3(1456); RAD50(10111); TCF7(6932); FLJ37562 |
| | | (134553); FLJ41603(389337); UNC5A(90249); MCC(4163); FLJ44796 |
| | | (401209); YIPF5(81555); FAT2(2196); SPARC(6678); FBXW11(23291); |
| | | RNF182(221687); HIST1H2AG(8969); TNF(7124); PARC(23113); FOXO3A |
| | | (2309); KIAA1244(57221); DST(667); DJ122O8.2(57226); SGK(6446); M |

Table 4-119

| | | |
|---|---|---|
| | | AP3K5(4217); EGFR(1956); FZD1(8321); CNTNAP2(26047); TNKS(8658); |
| | | POLR3D(661); RHOBTB2(23221); TNFRSF10C(8794); FZD3(7976); UNC5D |
| | | (137970); FAM77D(286183); SULF1(23213); FZD6(8323); PINIX1(54984); |
| | | TRIM35(23087); ZNF704(619279); PAG1(55824); PMP2(5375); CCNE2 |
| | | (9134); TP53INP1(94241); EXT1(2131); ANKRD15(23189); INVS(27130); |
| | | PAPPA(5069); TOR1B(27348); TAF1L(138474); BAG1(573); FANCG |
| | | (2189); TNFSF8(944); TRAF1(7185); PPP6C(5537); ABO(28); ZMYND11 |
| | | (10771); CDC2(983); SGPL1(8879); MARVELD1(83742); BTRC(8945); PDCD11 |
| | | (22984); CSPG6(9126); ADARB2(105); RP11-142I17.1(26095); C10orf72 |
| | | (196740); ZWINT(11130); FRAT2(23401); SMNDC1(10285); ADAM12 |
| | | (8038); STIM1(6786); HIPK3(10114); SIPA1(6494); LOH11CR2A(4013); |
| | | FZD4(8322); SESN3(143686); MGC33948(160140); LTBR(4055); PTPN6 |
| | | (5777); PA2G4(5036); XPOT(11260); GAS2L3(283431); KNTC1(9735); STYK1 |
| | | (55359); KRAS(3845); ITPR2(3709); DDIT3(1649); C12orf61(283416); |
| | | LIN7A(8825); IGF1(3479); DIP13B(55198); CHFR(55743); SPATA13(221178); |
| | | CDK8(1024); BRCA2(675); RB1(5925); RFP2(10206); ING1(3621); FOXO1A |
| | | (2308); AKAP6(9472); CDKN3(1033); SLC24A4(123041); ACIN1 |
| | | (22985); LTBP2(4053); TGFB3(7043); CHES1(1112); BAG5(9529); C15orf41 |
| | | (84529); IL16(3603); FLJ32310(123624); IGF1R(3480); WDR72(256764); |
| | | SLTM(79811); CSNK1G1(53944); CALML4(91860); CCNF(899); KREMEN2 |
| | | (79412); DNAJA3(9093); CYLD(1540); KATNB1(10300); GSPT1(2935); |
| | | ABCC6(368); CSNK2A2(1459); RBM35B(80004); PAFAH1B1(5048); MAPK7 |
| | | (5598); ANKRD13B(124930); GOSR1(9527); NF1(4763); MAPT(4137); |
| | | CROP(51747); PPM1D(8493); PRKCA(5578); KPNA2(3838); SOX9(6662); |
| | | NALP1(22861); GPS2(2874); ARHGAP27(201176); MXRA7(439921); |
| | | DCC(1630); PHLPP(23239); SDCCAG33(10194); ROCK1(6093); ESCO1 |
| | | (114799); FAM59A(64762); TCEB3C(162699); TXNL1(9352); CDH19(28513); |
| | | CSNK1G2(1455); HMG20B(10362); ZNF440L(284390); CCNE1(898); |
| | | LOC400696(400696); SNF230(7773); FLJ26175(388566); ZBTB7A(51341); |
| | | ELAVL1(1994); MGC71805(403113); BRD4(23476); STK35(140901); CTNNBL1 |
| | | (56259); CTCFL(140690); DIDO1(11083); SON(6651); SEPT5(5413); |
| | | EP300(2033); SEPT3(55964); MAPK1(5594); CCNB3(85417); TAF1 |
| | | (6872); AFF2(2334); SMC1L1(8243); RP13-383K5.1(55285); GAB3(139716); |
| hsa-miR-600 | 2084; | SPSB1(80176); LOC148898(148898); MACF1(23499); KIAA0467(23334); |
| | | ATG4C(84938); FAM73A(374986); CDC14A(8556); RAP1A(5906); PEA15 |
| | | (8682); UHMK1(127933); DNM3(26052); RALGPS2(55103); C1orf99(339476); |
| | | PPP1R12B(4660); ADORA1(134); MDM4(4194); IL24(11009); PFKFB2 |
| | | (5208); TGFB2(7042); C1orf69(200205); C1orf150(148823); HES2(54626); |
| | | FAF1(11124); TTC22(55001); FUBP1(8880); GFI1(2672); WARS2(10352); |
| | | NOTCH2(4853); MCL1(4170); CD5L(922); DEDD(9191); ASPM(259266); |
| | | C1orf145(574407); FOSL2(2355); CRIM1(51232); PRKCE(5581); MSH2 |
| | | (4436); HSPC159(29094); CCT7(10574); CCNT2(905); RIF1(55183); PMS1 |
| | | (5378); CASP10(843); CREB1(1385); RBJ(51277); CARD12(58484); EDAR |
| | | (10913); ERCC3(2071); MGC4268(83607); TLK1(9874); KIAA1715(80856); |
| | | FRZB(2487); STK17B(9262); TNS1(7145); CUL3(8452); IRS1(3667); |
| | | TRIP12(9320); NR2C2(7182); PCAF(8850); PDCD6IP(10015); MOBP(4336); |
| | | LIMD1(8994); ARIH2(10425); PCNP(57092); TLOC1(7095); NAALADL2 |
| | | (254827); NEK4(6787); LRIG1(26018); VGLL3(389136); CGGBP1(8545); |
| | | SIAH2(6478); TBL1XR1(79718); CLDN1(9076); WHSC1(7468); HD(3064); |
| | | HCAP-G(64151); STIM2(57620); HIP2(3093); REST(5978); EREG(2069); |
| | | SEPT11(55752); FGF2(2247); NPY2R(4887); APBB2(323); DDIT4L |
| | | (115265); FGA(2243); CASP3(836); FAM105B(90268); NIPBL(25836); CARD6 |
| | | (84674); KIF2(3796); JMY(133746); RHOBTB3(22836); LOC91137(91137); |
| | | CSNK1G3(1456); ISOC1(51015); G3BP(10146); SEMA5A(9037); RICTOR |
| | | (253260); PRKAA1(5562); FGF10(2255); UNG2(10309); BIRC1(4671); |
| | | MCC(4163); ATG12(9140); IRF1(3659); ZCCHC10(54819); PPP2CA(5515); |
| | | CSNK1A1(1452); C5orf4(10826); CSNK2B(1460); PARC(23113); HECA |
| | | (51696); ZNF323(64288); DHX16(8449); TNFRSF21(27242); DST(667); |
| | | ATG5(9474); OSTM1(28962); CDC2L6(23097); PDCD2(5134); STK17A |
| | | (9263); ADCY1(107); DLX6(1750); BCAP26(55973); KIAA0265(23008); CYCS |
| | | (54205); HOXA10(3206); INHBA(3624); GLI3(2737); CDK6(1021); PPP1R3A |
| | | (5506); DLGAP2(9228); KBTBD11(9920); ESCO2(157570); BAG4(9530); |
| | | TACC1(6867); UBE2V2(7336); PTDSS1(9791); WISP1(8840); TNFRSF10B |
| | | (8795); TNFRSF10D(8798); MYST3(7994); PLAG1(5324); TP53IN |

Table 4-120

| | | |
|---|---|---|
| | | P1(94241); MTSS1(9788); ANKRD15(23189); DAPK1(1612); CKS2(1164); |
| | | RABGAP1(23637); CDKN2B(1030); KLF9(687); TRIM14(9830); STRBP |
| | | (55342); C9orf90(203245); C10orf9(219771); NUDT5(11164); PARD3(56288); |
| | | FZN8(8325); ZWINT(11130); DLG5(9231); SLC16A12(387700); TLL2 |
| | | (7093); SH3PXD2A(9644); C10orf46(143384); MKI67(4288); BIRC3(330); |
| | | ATM(472); MLL(4297); SC5DL(6309); CHEK1(1111); WT1(7460); LOC283219 |
| | | (283219); RAB30(27314); PPP2R1B(5519); FKBP4(2288); CDKN1B |
| | | (1027); PDE6H(5149); ETNK1(55500); MDM2(4193); CPSF6(11052); CRADD |
| | | (8738); GAS2L3(283431); JARID1A(5927); TNFRSF1A(7132); BCAT1 |
| | | (586); LOC196394(196394); SLC38A2(54407); MCRS1(10445); BTG1(694); |
| | | ARL1(400); GIT2(9815); ANAPC7(51434); ANAPC5(51433); MPHOSPH9 |
| | | (10198); CDK2AP1(8099); HSMPP8(54737); FGF9(2254); TNFRSF19 |
| | | (55504); BRCA2(675); RB1(5925); MTMR6(9107); STARD13(90627); CSNK1A1L |
| | | (122011); FOXO1A(2308); KIAA0853(23091); NEK3(4752); LOC283487 |
| | | (283487); CGRRF1(10668); UNQ9438(387990); PLEKHH1(57475); |
| | | VASH1(22846); KIAA1737(85457); TRAF3(7187); NOVA1(4857); LTBP2 |
| | | (4053); GTF2A1(2957); BCL11B(64919); CASC5(57082); FGF7(2252); TMOD3 |
| | | (29766); TCF12(6938); FEM1B(10116); KIAA1199(57214); MYEF2(50804); |
| | | MYO5A(4644); ARPP-19(10776); CCPG1(9236); PRTG(283659); BNIP2 |
| | | (663); FLJ22795(80154); CCNF(899); RBL2(5934); SLC7A6(9057); AXIN1 |
| | | (8312); C16orf5(29965); CIAPIN1(57019); SMPD3(55512); PAFAH1B1 |
| | | (5048); MYOCD(93649); CROP(51747); NME1(4830); PRKCA(5578); DDX52 |
| | | (11056); PTRH2(51651); ERN1(2081); DTNA(1837); MAPRE2(10982); |
| | | ROCK1(6063); TXNL4A(10907); PDE4A(5141); DNM2(1785); AXL(558); |
| | | BRD4(23476); BBC3(27113); KIF3B(9371); STK4(6789); SLC2A10(81031); |
| | | PTPN1(5770); VAPB(9217); PDRG1(81572); PTPRT(11122); UBE2V1 |
| | | (7335); CTCFL(140690); USP16(10600); CTB-1048E9.5(402055); NF2(4771); |
| | | MCM5(4174); TNRC6B(23112); EP300(2083); SEPT3(55964); C22orf9 |
| | | (23313); RP1-112K5.2(90121); MAGEH1(28986); ZC3H12B(340554); ARMCX4 |
| | | (158947); PRPS1(5631); RP6-213H19.1(51765); FIGF(2277); SMC1L1 |
| | | (8243); RNF12(51132); BTK(695); SEPT6(23157); |
| hsa-miR-601 | 2085; | TNFRSF1B(7133); ATPIF1(93974); TXLNA(200081); MACF1(28499); AK5 |
| | | (26289); PRKACB(5567); BCL9(607); C1orf21(81563); ADORA1(134); IL24 |
| | | (11009); TRAF5(7188); E2F2(1870); CD5L(922); DEDD(9191); CFLAR |
| | | (8837); CD28(940); RTKN(6242); PAX3(5077); CUL3(8452); FANCD2(2177); |
| | | EAF2(55840); ACPP(55); AXUD1(64651); DNASE1L3(1776); FHIT(2272); |
| | | ATR(545); CCNG2(901); CASP3(836); APC(324); LHFPL2(10184); CD14 |
| | | (929); GABRB2(2561); PTP4A1(7803); TCF21(6943); SASH1(23328); |
| | | TNFRSF21(27242); DST(667); PLAGL1(5325); CUL1(8454); CARD4(10392); |
| | | ELMO1(9844); CDK6(1021); HSPC047(29060); CLN8(2055); BNIP3L |
| | | (665); CA2(760); TNFRSF10B(8795); UNC13B(10497); NTRK2(4615); TRAF2 |
| | | (7186); IFNW1(3467); AKNA(80709); PPP6C(5537); SIRT1(23411); UNC5B |
| | | (219699); SUFU(51684); CSPG6(9126); PARD3(56288); RP11-142I17.1 |
| | | (26095); HPS1(3257); EXT2(2132); FADD(8772); HRASLS3(11145); BCL2L14 |
| | | (79370); FLJ20489(55652); PDE1B(5153); MDM2(4193); PTPN11 |
| | | (5781); ING4(51147); LALBA(3906); SSH1(54434); GPR81(27198); FGF9 |
| | | (2254); BRCA2(675); LIG4(3981); BCL2L2(599); DPF3(8110); MOAP1(64112); |
| | | PPP1R13B(23368); GOLGA8E(390535); GOLGA8G(283768); LOC440295 |
| | | (440295); GOLGA8B(440270); ARMC5(79798); E2F4(1874); PKMYT1 |
| | | (9088); ABCC6(368); DNAJA2(10294); CSNK2A2(1459); TRADD(8717); |
| | | SMPDS(55512); TERF2(7014); ALOX15B(247); NALP1(22861); ZBTB4 |
| | | (57659); MPO(4353); MXRA7(439921); CABLES1(91768); STATIP1(55250); |
| | | MALT1(10892); PHLPP(23239); MAP3K10(4294); HIF3A(64344); CRX |
| | | (1406); TGFB1(7040); CGB7(94027); CST9(128822); Kua(387521); CTCFL |
| | | (140690); CABLES2(81928); SON(6651); APP(351); NF2(4771); SEPT3 |
| | | (55964); MAPK1(5594); CARD10(29775); ZBTB33(10009); SMC1L1(8243); |
| hsa-miR-604 | 2086; | TP73(7161); DFFB(1677); ESPN(83715); H6PD(9563); TNFRSF1B(7133); |
| | | PAX7(5081); NBL1(4681); KHDRBS1(10657); PLK3(1263); TNFRSF18 |
| | | (8784); CDC2L2(985); E2F2(1870); TESK2(10420); TAL1(6886); BCL10(8915); |
| | | FH(2271); HPCAL1(3241); RHOB(388); MAPRE3(22924); TRIM54(57159); |
| | | PPP1CB(5500); PRKCE(5581); HK2(3099); TCF7L1(83439); MAL |
| | | (4118); KCNIP3(30818); FZD7(8324); CD28(940); INHA(3623); COL4A3(1285); |
| | | PSMD1(5707); PTMA(5757); BOK(666); CGREF1(10669); EIF2AK2 |
| | | (5610); RTN4(57142); PAX3(5077); CUL3(8452); HDAC4(9756); VHL(742 |

Table 4-121

| | | |
|---|---|---|
| | | 8); PCAF(8850); FAIM(55179); NME6(10201); ATR(545); SIAH2(6478); HD |
| | | (3064); EREG(2069); SEPT11(55752); FGF5(2250); NFKB1(4790); TLR2 |
| | | (7097); APBB2(323); CCNI(10983); LEF1(51176); TCF7(6932); UNG2(10309); |
| | | BIRC1(4671); SEMA6A(57556); NRG2(9542); CD14(929); CD74(972); |
| | | RIPK1(8737); MAPK13(5603); VEGF(7422); FOXO3A(2309); HECA(51696); |
| | | SASH1(23328); DHX16(8449); MDC1(9656); IER3(8870); CUL7(9820); |
| | | MCM3(4172); PLAGL1(5325); LOC401286(401286); FLJ20397(54919); |
| | | STAG3(10734); CUTL1(1523); CASP2(835); CARD4(10392); ELMO1(9844); |
| | | YWHAG(7532); MCM7(4176); E2F5(1875); EBAG9(9166); LRRC14(9684); |
| | | C8orf13(83648); TNFRSF10D(8793); DUSP4(1846); CEBPD(1052); |
| | | TP53INP1(94241); EXT1(2131); INVS(27130); CDK9(1025); ABL1(25); CDKN2A |
| | | (1029); FANCG(2189); GAS1(2619); TRIM14(9830); CIZ1(25792); |
| | | CARD9(64170); SGPL1(8879); UNC5B(219699); CHST3(9469); KIF11(3832); |
| | | FRAT1(10023); SUFU(51684); PDCD11(22984); C10orf39(282973); CUL2 |
| | | (8453); DLG5(9231); SFRP5(6425); FGF8(2253); C10orf46(143384); BNIP3 |
| | | (664); STIM1(6786); WEE1(7465); HIPK3(10114); SSSCA1(10534); |
| | | SIPA1(6494); LRP5(4041); FADD(8772); ATM(472); IGF2(3481); CDKN1C |
| | | (1028); APBB1(322); DKK3(27122); MAP3K11(4296); CLCF1(23529); MTL5 |
| | | (9633); FLJ42258(440049); FGF4(2249); FZD4(8322); SESN3(143686); |
| | | BCL9L(283149); LTBR(4055); PTMS(5763); CDKN1B(1027); PDE1B(5153); |
| | | MDM2(4193); SFRS8(6433); ING4(51147); HDAC7A(51564); FAIM2 |
| | | (23017); PCTK2(5128); MSI1(4440); CDK2AP1(8099); CHFR(55743); HSMPP8 |
| | | (54737); ING1(3621); LATS2(26524); SLC7A1(6541); FOXO1A(2308); |
| | | GALNTL1(57452); TRAF3(7187); CIDEB(27141); STRN3(29966); NFKBIA |
| | | (4792); SPTB(6710); FLJ39779(400223); LTBP2(4053); PPP1R13B |
| | | (23368); BMF(90427); PDCD7(10081); TSC2(7249); TRAF7(84231); CCNF |
| | | (899); FLYWCH1(84256); DNASE1(1773); MGC2654(79091); PLK1(5347); |
| | | CARD15(64127); KATNB1(10300); WWP2(11060); ERN2(10595); SMPD3 |
| | | (55512); PCOLN3(5119); HIC1(3090); RUTBC1(9905); TNFSF12(8742); |
| | | NTN1(9423); RAI1(10743); MAPK7(5598); RAB11FIP4(84440); SOCS7 |
| | | (30837); FZD2(2535); FMNL1(752); MAPT(4137); NGFR(4804); TLK2(11011); |
| | | PRKCA(5578); CARD14(79092); ANAPC11(51529); MNT(4335); GAS2L2 |
| | | (246176); MPO(4353); SEPT4(5414); SOCS3(9021); CBX4(8535); TBC1D16 |
| | | (125058); MAFG(4097); CABLES1(91768); MAPRE2(10982); PHLPP |
| | | (23239); GRIN3B(116444); STK11(6794); MUM1(84939); APC2(10297); |
| | | REEP6(92840); FZR1(51343); ATCAY(85300); CHAF1A(10036); MAP2K7 |
| | | (5609); DNM2(1785); UBA52(7311); KCTD15(79047); APLP1(333); WDR62 |
| | | (284403); MAP3K10(4294); AXL(558); BCL3(602); APOE(348); SPHK2 |
| | | (56848); PPP2R1A(5518); AURKC(6795); GNG7(2788); AES(166); DAPK3 |
| | | (1613); CD209(30835); CDKN2D(1032); DPF1(8193); GRIK5(2901); BBC3 |
| | | (27113); KLK10(5655); PARD6B(84612); BIRC7(79444); BCL2L1(598); RBL1 |
| | | (5933); UBE2V1(7335); CABLES2(81928); DIDO1(11083); SON(6651); |
| | | PDXK(8566); SNF1LK(150094); ITGB2(3689); CDC45L(8318); SEPT5 |
| | | (5413); SMARCB1(6598); ADORA2A(135); GAS2L1(10634); NF2(4771); MTP18 |
| | | (51537); EP300(2033); SEPT3(55964); FBLN1(2192); PPARA(5465); |
| | | MAPK8IP2(23542); MAPK1(5594); PPM1F(9647); MN1(4330); SSTR3(6753); |
| | | CARD10(29775); PDGFB(5155); NHP2L1(4809); CCNB3(85417); TAF1 |
| | | (6872); NGFRAP1(27018); HCFC1(3054); |
| hsa-miR-605 | 2087; | CDC42(998); EPB41(2035); KHDRBS1(10657); RBBP4(5928); MACF1(23499); |
| | | PLK3(1263); NSUN4(387338); BTF3L4(91408); RAP1A(5906); PTGFRN |
| | | (5738); BCL9(607); BNIPL(149428); TUFT1(7286); FCGR2B(2213); |
| | | ATF6(22926); UHMK1(127933); TOR1AIP1(26092); PFKFB2(5208); TRAF5 |
| | | (7188); TGFB2(7042); ICMT(23463); TNFRSF9(3604); CTNNBIP1(56998); |
| | | FRAP1(2475); CASP9(842); ALDH4A1(8659); TTMB(399474); YARS |
| | | (8565); PIK3R3(8503); DAB1(1600); NOTCH2(4853); RORC(6097); PYGO2 |
| | | (90780); SYT2(127833); IRF6(3664); WDR26(80232); ENAH(55740); RNF144 |
| | | (9781); GREB1(9687); UBXD4(165324); SLC30A6(55676); BIRC6 |
| | | (57448); RHOQ(23433); ACTR2(10097); ANTXR1(84168); KCNIP3(30818); |
| | | MAP4K4(9448); MGC5509(79074); TSN(7247); TAIP-2(80034); CD28(940); |
| | | ALS2CR19(117583); NRP2(8828); CREB1(1385); LOC129607(129607); |
| | | CARD12(58484); RAB1A(5861); SPRED2(200734); TGFA(7039); EDAR |
| | | (10913); SEPT10(151011); ANAPC1(64682); ERCC3(2071); HS6ST1(9394); |
| | | STAM2(10254); PTD004(29789); TRAK2(66008); FLJ43879(401039); |
| | | NR1D2(9975); GPD1L(23171); PDCD6IP(10015); DLEC1(9940); CTNNB1 |
| | | (1499); TRAK1(22906); FAM55C(91775); ZDHHC23(254887); ACAD9(2 |

Table 4-122

8976); FAIM(55179); RNF7(9616); SENP2(59343); TP73L(8626); RAF1(5894);
RPL32(6161); SH3BP5(9467); THRB(7068); PRKAR2A(5576); MAGI1
(9223); EIF4E3(317649); VGLL3(389136); DCBLD2(131566); CD47(961);
TMCC1(23023); PIK3CB(5291); TFDP2(7029); MGC40579(256356); ATR
(545); WHSC1(7468); HD(3064); HCAP-G(64151); RUFY3(22902); SEPT11
(55752); FRAS1(80144); METAP1(23173); CLCN3(1182); STOX2(56977);
APBB2(323); CLOCK(9575); UGT2B15(7366); FAM13A1(10144); DKK2
(27123); LEF1(51176); ANKRD50(57182); SLC7A11(23657); TBC1D9
(23158); MFAP3L(9848); MTRR(4552); NLN(57486); LOC153561(153561);
MAP1B(4131); SCAMP1(9522); CAST(831); LOC90355(90355); CSNK1G3
(1456); NDST1(3340); PRLR(5618); P18SRP(285672); BIRC1(4671);
ENC1(8507); LIX1(167410); MCC(4163); CSNK1A1(1452); FBXW11(23291);
TCF19(6941); AIF1(199); TPBG(7162); FOXO3A(2309); TNFAIP3(7128);
PHACTR2(9749); PLG(5340); QKI(9444); ATXN1(6310); MDC1(9656);
DAXX(1616); TRAM2(9697); DST(667); ATG5(9474); SEC63(11231); FRK
(2444); ARHGAP18(93663); PLAGL1(5325); LATS1(9113); PDCD2(5134);
FOXK1(221937); AHR(196); SEPT7(989); EGFR(1956); SVH(83787); CENTG3
(116988); TRIAD3(54476); PMS2(5395); ETV1(2115); CYCS(54205);
CARD4(10392); SFRP4(6424); IGFBP3(3486); YWHAG(7532); CDK6
(1021); FAM3C(10447); GCC1(79571); MTPN(136319); HIPK2(28996); KIAA0738
(9747); MTMR9(66036); ZNF705CP(389631); TNFRSF10C(8794);
FZD3(7976); TACC1(6867); LZTS1(11178); ADRA1A(148); SFRP1(6422);
IMPAD1(54928); RUNX1T1(862); EXT1(2131); TMEM65(157378); JAK2
(3717); INVS(27130); PAPPA(5069); ZNF297B(23099); CDKN2B(1030); C9orf100
(84904); PTCH(5727); TRIM14(9830); GABBR2(9568); CTNNAL1
(8727); C9orf5(23731); ROD1(9991); TRAF1(7185); TSC1(7248); ABO(28);
QSCN6L1(169714); DHTKD1(55526); HSPA14(51182); PTEN(5728); HECTD2
(143279); KIF11(3832); SCD(6319); TCF7L2(6934); GPR26(2849);
SFMBT2(57713); C10orf72(196740); DLG5(9231); POLR3A(11128); C10orf46
(143384); TIAL1(7073); MKI67(4288); IPO7(10527); PDE3B(5140); TMEM16C
(63982); EXT2(2132); STX3A(6809); VEGFB(7423); NPAS4(266743);
LRP5(4041); FADD(8772); AMOTL1(154810); CUL5(8065); ATM(472);
PAFAH1B2(5049); ARHGEF12(23365); FLI1(2313); CDKN1C(1028);
MRPL17(63875); DKK3(27122); CDCA5(113130); LTBP3(4054); FCHSD2
(9873); FZD4(8322); NPAT(4863); POU2AF1(5450); IGSF4(23705); BCL9L
(283149); CCND2(894); PTPN6(5777); GABARAPL1(23710); ETV6(2120);
REP15(387849); MGC50559(254013); FLJ20489(55652); SLC16A7(9194)
DYRK2(8445); MDM2(4193); LGR5(8549); APAF1(317); KIAA1853
(84530); KIAA0152(9761); KNTC1(9735); BRI3BP(140707); TMEM132B
(114795); FZD10(11211); JARID1A(5927); BCAT1(586); KRAS(3845); ZNF641
(121274); FAIM2(23017); CBX5(23468); SMARCC2(6601); PAWR(5074);
HRK(8739); CDK2AP1(8099); MRP63(78988); FGF9(2254); ATP8A2(51761);
CDK8(1024); BRCA2(675); ARHGEF7(8874); CDC16(8881); CSNK1A1L
(122011); FOXO1A(2308); RP11-5G9.1(160857); BCL2L2(599); KIAA1333
(55632); TXNDC(81542); PSEN1(5663); C14orf111(51077); VASH1
(22846); FLRT2(23768); OTUB2(78990); TRAF3(7187); GCH1(2643); NEK9
(91754); TMED10(10972); C14orf1(11161); TGFB3(7043); MAPK6(5597);
AQP9(366); IL16(3603); AKAP13(11214); ST8SIA2(8128); LRRK1(79705);
UBE3A(7337); SUHW4(54816); CSNK1G1(53944); CCNF(899); KIAA0251
(23042); PLK1(5347); MGC13024(93129); CCL22(6367); AXIN1(8312);
NME3(4832); C16orf5(29965); GSPT1(2935); MIR16(51573); USP31
(57478); FTS(64400); CMTM4(146223); TXNL4B(54957); ATBF1(463); ZCCHC14
(23174); RPA1(6117); PAFAH1B1(5048); NLK(51701); CPD(1362);
GOSR1(9527); DKFZp667M2411(147172); NF1(4763); DHX8(1659); MAPT
(4137); ITGB3(3690); NGFR(4804); MYST2(11143); CROP(51747); PCTP
(58488); SPHK1(8877); ZZEF1(23140); NALP1(22861); SLC4A1(6521);
KIAA0553(23131); SPOP(8405); MRPL27(51264); SPAG9(9043); TEX2(55852);
TK1(7083); TBC1D16(125058); CSNK1D(1453); RAB12(201475); ANKRD30B
(374860); RNF138(51444); MAPRE2(10982); MAPK4(5596); PHLPP
(23239); DSC2(1824); BCL2(596); CSNK1G2(1455); PDE4A(5141);
LDLR(3949); CCNE1(898); PLEKHG2(64857); MAP3K10(4294); SPIB(6689);
ZNF304(57343); AES(166); FUT5(2527); MGC19604(112812); DKFZp686I1569
(400720); TRIB3(57761); PTPNS1(140885); PLCB1(23236); MAPRE1
(22919); UBE2C(11065); PARD6B(84612); VAPB(9217); CD93(22918);
GGTL3(2686); RBL1(5933); PREX1(57580); AURKA(6790); CABLES

Table 4-123

| | | |
|---|---|---|
| | | 2(81928); DIDO1(11083); MX1(4599); RUNX1(861); BRWD1(54014); PLAC4 |
| | | (191585); C21orf29(54084); POFUT2(23275); BCL2L13(23786); CTB-1048E9.5 |
| | | (402055); KREMEN1(83999); NF2(4771); CABP7(164633); H1F0 |
| | | (3005); TNRC6B(23112); MN1(4330); TOB2(10766); C22orf9(23313); SMC1L2 |
| | | (27127); CXorf15(55787); CNKSR2(22866); ARAF(369); ZC3H12B(340554); |
| | | MLLT7(4303); PCDH11X(27328); STAG2(10735); SMC1L1(8243); |
| | | PHF8(23133); RP11-130N24.1(340533); RP11-217H1.1(84061); CHM(1121); |
| | | AMMECR1(9949); SEPT6(23157); PCDH11Y(83259); |
| hsa-miR-606 | 2088; | SAMD11(148398); KIAA1026(23254); RNF11(26994); C1orf83(127428); PRKAA2 |
| | | (5563); SH3GLB1(51100); FNBP1L(54874); CDC14A(8556); GNAI3 |
| | | (2773); RAP1A(5906); RBM8A(9939); PEA15(8682); UHMK1(127933); |
| | | RGS2(5997); TRAF5(7188); FBXO28(23219); FUSIP1(10772); BCL10(8915); |
| | | GBP4(115361); TRIM33(51592); DENND2C(163259); NRAS(4893); NOTCH2 |
| | | (4853); ASPM(259266); RIPK5(25778); SOX11(6664); SELI(85465); |
| | | PPP1CB(5500); BIRC6(57448); C2orf30(27248); BRRN1(23397); RIF1 |
| | | (55183); TAIP-2(80034); PPIG(9360); FLJ13096(80067); CYBRD1(79901); |
| | | CD28(940); RPE(6120); COL4A3(1285); PTMA(5757); KIDINS220(57498); |
| | | CARD12(58484); TGFA(7039); ORC4L(5000); ACVR1(90); METTL5(29081); |
| | | FLJ13946(92104); SESTD1(91404); STAT1(6772); CLK1(1195); BARD1 |
| | | (580); USP37(57695); PDCD6IP(10015); GNL3(26354); EAF2(55840); |
| | | MBNL1(4154); SMC4L1(10051); ATP11B(23200); OPA1(4976); SEC22L3 |
| | | (9117); MAP4(4134); NEK4(6787); IL17RD(54756); VGLL3(389136); KPNA1 |
| | | (3836); ATR(545); TBL1XR1(79718); CPEB2(132864); CD38(952); ANAPC4 |
| | | (29945); RBPSUH(3516); GUF1(60558); MOBKL1A(92597); SLC4A4 |
| | | (8671); C4orf12(404201); FGF2(2247); PLK4(10733); SMARCA5(8467); |
| | | TMEM128(85013); FAM44A(259282); GABRA4(2557); BTC(685); RCHY1 |
| | | (25898); HNRPDL(9987); MAD2L1(4085); ANKRD50(57182); C4orf13 |
| | | (84068); SFRP2(6423); NEK1(4750); MFAP3L(9848); NIPBL(25838); UTP15 |
| | | (84135); POLK(51426); F2R(2149); SCAMP1(9522); APC(324); DCP2(167227); |
| | | CSNK1G3(1456); RAD50(10111); TCF7(6932); PRLR(5618); BIRC1 |
| | | (4671); MGC23909(153339); C5orf13(9315); MCC(4163); RAPGEF6(51735); |
| | | IRF1(3659); GABRB2(2561); CDKN1A(1026); C6orf139(55166); IL17 |
| | | (3605); WTAP(9589); QKI(9444); MDC1(9656); DAXX(1616); NUDT3(11165); |
| | | DST(667); CCNC(892); SIM1(6492); ATG5(9474); SESN1(27244); STX7 |
| | | (8417); IL20RA(53832); LATS1(9113); PDCD2(5134); EGFR(1956); FZD9 |
| | | (8326); ZKSCAN1(7586); BCAP29(55973); CAV2(858); CASP2(835); TWISTNB |
| | | (221830); HNRPA2B1(3181); INHBA(3624); LOC441233(441233); |
| | | CDK6(1021); TFPI2(7980); FLJ42280(401388); ZC3HC1(51530); ZDHHC2 |
| | | (51201); BNIP3L(665); ESCO2(157570); FZD3(7976); TERF1(7013); |
| | | C8orf53(84294); PHF20L1(51105); STC1(6781); SFRP1(6422); PLAG1(5324); |
| | | EYA1(2138); FAM82B(51115); RUNX1T1(862); STK3(6788); RAD21 |
| | | (5885); MTSS1(9788); CDC37L1(55664); NTRK2(4915); MRRF(92399); RALGPS1 |
| | | (9649); TAF1L(138474); KLF9(687); TSC1(7248); ZMYND11(10771); |
| | | NET1(10276); LOC389936(389936); ZNF37A(7587); ZNF485(220992); |
| | | MARCH5(54708); KIF11(3832); BTRC(8945); FLJ45187(387640); PARD3 |
| | | (56288); FAM13C1(220965); KCNMA1(3778); DCLRE1A(9937); KIAA1598 |
| | | (57698); RAB11FIP2(22841); WEE1(7465); HIPK3(10114); DPF2(5977); |
| | | CCND1(595); FADD(8772); YAP1(10413); CUL5(8065); ATM(472); FDX1 |
| | | (2230); CHEK1(1111); PPP1CA(5499); PAK1(5058); SLC36A4(120103); |
| | | SESN3(143686); TMEM123(114908); KBTBD3(143879); PPP2R1B(5519); |
| | | CCND2(894); MDM2(4193); CPSF6(11052); TMTC3(160418); NUDT4(11163); |
| | | VEZT(55591); SCYL2(55681); TDG(6996); FZD10(11211); JARID1A |
| | | (5927); FLJ10292(55110); LRP6(4040); BCAT1(586); HDAC7A(51564); |
| | | MCRS1(10445); PHLDA1(22822); ARL1(400); ANAPC7(51434); MPHOSPH9 |
| | | (10198); CDK2AP1(8099); TNFRSF19(55504); RFP2(10206); ITR(160897); |
| | | ING1(3621); KIAA1008(22894); LIG4(3981); ARHGAP5(394); PNN |
| | | (5411); TXNDC(81542); SOCS4(122809); SLC39A9(55334); HNRPC(3183); |
| | | TINF2(26277); SNX6(58533); WDR22(8816); LTBP2(4053); GTF2A1(2957); |
| | | SEL1L(6400); BAG5(9529); GREM1(26585); MYEF2(50804); ARPP-19 |
| | | (10776); PRTG(283659); SUHW4(54816); SLTM(79811); BNIP2(663); MESDC2 |
| | | (23184); CYLD(1540); NFAT5(10725); GGA2(23062); FTS(64400); |
| | | NF1(4763); PPM1E(22843); PPM1D(8493); TLK2(11011); NUFIP2(57532); |
| | | BRCA1(672); HELZ(9931); ACOX1(51); CLUL1(27098); DSG3(1830); MALT1 |
| | | (10892); TNFRSF11A(8792); DOK6(220164); ROCK1(6093); ESCO1 |
| | | (114799); P15RS(55197); SMAD2(4087); BCL2(596); C18orf4(92126); ZA |

Table 4-124

| | | |
|---|---|---|
| | | DH2(284273); APOE(348); PPP2R1A(5518); ZNF625(90589); ZNF443(10224); |
| | | ZNF585A(199704); PPP1R13L(10848); CARD8(22900); MCM8(84515); |
| | | KIAA1219(57148); C20orf100(84969); C20orf121(79183); STK4(6789); |
| | | PARD6B(84612); RAB22A(57403); C20orf177(63939); CSNK2A1(1457); |
| | | RNF24(11237); CABLES2(81928); BACH1(571); BRWD1(54014); ZNF295 |
| | | (49854); H1F0(3005); EP300(2033); MN1(4330); ZXDB(158586); TAF1(6872); |
| | | NGFRAP1(27018); CXorf39(139231); BIRC4(331); RP6-213H19.1(51765); |
| | | CXorf20(139105); DMD(1756); AMMECR1(9949); PDCD8(9131); DAZ2 |
| | | (57055); |
| hsa-miR-609 | 2089; | EFHD2(79180); ARID1A(8289); RBBP4(5928); KIAA0467(23334); CDKN2C |
| | | (1031); RAP1A(5906); IGSF4B(57863); C1orf21(81563); CNTN2(6900); |
| | | IL19(29949); TGFB2(7042); C1orf95(375057); HES2(54626); CTNNBIP1 |
| | | (56998); FRAP1(2475); CASP9(842); RCC2(55920); NOTCH2(4853); PYGO2 |
| | | (90780); ASPM(259266); PRKCE(5581); HK2(3099); BRRN1(23397); ZAK |
| | | (51776); CASP10(843); CGREF1(10669); REV1L(51455); EDAR(10913); |
| | | IL1A(3552); FRZB(2487); PAX3(5077); PCAF(8850); RBM15B(29890); |
| | | TP73L(8626); PB1(55193); MAGI1(9223); GSK3B(2932); TMCC1(23023); |
| | | STAG1(10274); HD(3064); KLHL5(51088); APBB2(323); LEF1(51176); NIPBL |
| | | (25836); MAP1B(4131); DAP(1611); PRLR(5618); UNG2(10309); FLJ44796 |
| | | (401209); DDX41(51428); ZNF192(7745); CSNK2B(1460); TTBK1 |
| | | (84630); GRIK2(2898); TNFAIP3(7128); PPP1R10(5514); IER3(8870); CUL7 |
| | | (9820); DST(667); UBE2D4(51619); EGFR(1956); SFRP4(6424); INHBA |
| | | (3624); CDK6(1021); BRAF(673); ESCO2(157570); UNC5D(137970); MYC |
| | | (4609); TNFRSF10B(8795); SFRP1(6422); MTSS1(9788); ARC(23237); |
| | | ANKRD15(23189); JAK2(3717); ALDH1B1(219); ABL1(25); POMT1(10585); |
| | | FAM69B(138311); TAF1L(138474); PTCH(5727); GRIN3A(116443); TRAF1 |
| | | (7185); CIZ1(25792); BRD3(8019); ANAPC2(29882); ZMYND11(10771); |
| | | SIRT1(23411); SGPL1(8879); BTRC(8945); DOCK1(1793); ADARB2 |
| | | (105); SFMBT2(57713); CUL2(8453); FGFR2(2263); MKI67(4288); BNIP3 |
| | | (664); IFTTM1(8519); INCENP(3619); LGALS12(85329); SIPA1(6494); IGF2 |
| | | (3481); RAG2(5897); MAP3K11(4296); FLJ90834(283135); BCL9L(283149); |
| | | CCND2(894); CNAP1(9918); BCL2L14(79370); SSPN(8082); DNM1L |
| | | (10059); ESPL1(9700); MBD6(114785); APAF1(317); C12orf23(90488); BTBD11 |
| | | (121551); FAM101A(144347); RAD52(5893); C12orf22(81566); ZNF385 |
| | | (25946); GLS2(27165); CDK4(1019); PAWR(5074); TNFRSF19(55504); |
| | | BRCA2(675); RB1(5925); RFP2(10206); FLJ40296(122183); TFDP1(7027); |
| | | CSNK1A1L(122011); KBTBD7(84078); NEK3(4752); LIG4(3981); PNN |
| | | (5411); ACIN1(22985); CIDEB(27141); MAMDC1(161357); CHES1(1112); |
| | | PPP1R13B(23368); FGF7(2252); NDN(4692); SLC12A6(9990); CSNK1G1 |
| | | (53944); KATNB1(10300); CDK10(8558); FAM100A(124402); ERN2(10595); |
| | | FTS(64400); SMPD3(55512); ALOX12(239); MAPK7(5598); NF1(4763); |
| | | FZD2(2535); MAPT(4137); PRKCA(5578); CARD14(79092); ANAPC11 |
| | | (51529); MNT(4335); SPAG5(10615); GAS2L2(246176); TBC1D3C(414060); |
| | | BRCA1(672); ERN1(2081); CSNK1D(1453); MAPK4(5596); DCC(1630); |
| | | MBP(4155); NDUFA13(51079); CCNE1(898); AXL(558); BCL3(602); AES |
| | | (166); MATK(4145); SLC25A23(79085); ELAVL1(1994); PRDX2(7001); |
| | | HAPLN4(404037); GGN(199720); NUMBL(9253); ERF(2077); ZNF614(80110); |
| | | LOC284296(284296); STK35(140901); C20orf38(55304); CEP250(11190); |
| | | FOXA2(3170); RP5-1054A22.3(85449); AURKA(6790); CTCFL(140690); |
| | | BTBD4(140685); SON(6651); CHAF1B(8208); DSCAM(1826); SUSD2 |
| | | (56241); MCM5(4174); MAPK8IP2(23542); FLJ21125(79680); MAPK1 |
| | | (5594); PPM1F(9647); MN1(4330); RBM9(23543); PCTK1(5127); CCNB3 |
| | | (85417); TAF1(6872); H2AFB1(474382); SMC1L1(8243); HCFC1(3054); MECP2 |
| | | (4204); |
| hsa-miR-610 | 2090; | TNFRSF1B(7133); PLK3(1263); UHMK1(127933); CIAS1(114548); TMEM48 |
| | | (55706); EVI5(7813); F11R(50848); RHOQ(23433); MSH2(4436); HTRA2 |
| | | (27429); MRPL19(9801); RIF1(55183); RPE(6120); ASB1(51665); CDC42EP3 |
| | | (10602); FLJ23861(151050); HDAC4(9759); NKTR(4820); ZNF660 |
| | | (285349); APPL(26060); PCNP(57092); FAM55C(91775); DVL3(1857); PSMD2 |
| | | (5708); SENP2(59343); SRGAP3(9901); ZCSL2(285381); SMARCC1 |
| | | (6599); DNASE1L3(1776); ATR(545); LRRC15(131578); CD38(952); ANAPC4 |
| | | (29945); CCNG2(901); FLJ20032(54790); FGF2(2247); GALNT7(51809); |
| | | UGDH(7358); DNAJB14(79982); MARCH1(55016); DNAJA5(134218); |
| | | F2R(2149); LNPEP(4012); APC(324); RAD50(10111); MAML1(9794); PRLR |
| | | (5618); OSRF(23548); MCC(4163); FGF1(2246); ZNF300(91975); RIP |

Table 4-125

| | | |
|---|---|---|
| | | K1(8737); SENP6(26054); ATG5(9474); SGK(6446); BCLAF1(9774); MAP3K5 |
| | | (4217); HDAC9(9734); EGFR(1956); BCAP29(55973); CALU(813); MKLN1 |
| | | (4289); CASP2(835); DGKB(1607); INHBA(3624); CREB3L2(64764); |
| | | LMBR1(64327); FAM62B(57488); PDLIM2(64236); TACC1(6867); SLC30A8 |
| | | (169026); ZHX2(22882); WISP1(8840); XKR5(389610); TNFRSF10B |
| | | (8795); DUSP4(1846); RRM2B(50484); JAK2(3717); DAPK1(1612); TAF1L |
| | | (138474); FANCC(2176); BTRC(8945); GPR26(2849); NUDT5(11164); C10orf46 |
| | | (143384); EHF(26298); API5(8539); PANX1(24145); TBRG1(84897); |
| | | HRAS(3265); FZD4(8322); SESN3(143686); PPP2R1B(5519); CCND2(894); |
| | | ETNK1(55500); ZNFN1A4(64375); LNK(10019); OAS2(4939); LRP6(4040); |
| | | BCAT1(586); FAIM2(23017); POU6F1(5463); NT5DC3(51559); NUAK1 |
| | | (9891); ATP8A2(51761); CUL4A(8451); BCL2L2(599); KIAA1737(85457); |
| | | MOAP1(64112); BAG5(9529); BUB1B(701); PAQR5(54852); LOC440295 |
| | | (440295); AVEN(57099); BNIP2(663); CSNK1G1(53944); UBPH(56061); |
| | | PKMYT1(9088); SIAH1(6477); APPBP1(8883); SLC7A6OS(84138); HIC1 |
| | | (3090); PAFAH1B1(5048); NF1(4763); MSI2(124540); SPAG5(10615); PIP5K2B |
| | | (8396); TWSG1(57045); PHLPP(23239); ROCK1(6093); OR7D2(162998); |
| | | ATF5(22809); CARD8(22900); TRIB3(57761); KIAA1219(57148); PCNA |
| | | (5111); RBL1(5933); UBE2V1(7335); SON(6651); TMEM1(7109); UBE2G2 |
| | | (7327); BCL2L13(23786); RAB36(9609); TIMP3(7078); MN1(4330); TOB2 |
| | | (10766); YIPF6(286451); TAF1(6872); SPIN2(54466); EDA2R(60401); |
| | | ARHGEF6(9459); |
| hsa-miR-611 | 2091; | DFFB(1677); TNFRSF1B(7133); C1orf113(79729); DAP3(7818); G0S2(50486); |
| | | CIAS1(114548); CDC2L2(985); CHD5(26038); RCC2(55920); SEMA6C |
| | | (10500); FH(2271); HTRA2(27429); BRRN1(23397); FLJ43374(377007); |
| | | ASB1(51665); BOK(666); RTN4(57142); HD(3064); VEGFC(7424); NIPBL |
| | | (25836); UNC5A(90249); DAP(1611); PRLR(5618); FGF1(2246); PDLIM7 |
| | | (9260); TCF19(6941); CSNK2B(1460); PARC(23113); HECA(51696); IER3 |
| | | (8870); FRK(2444); SGK(6446); BCLAF1(9774); LATS1(9113); FZD1(8321); |
| | | PMS2(5395); ELMO1(9844); UNC5D(137970); TACC1(6867); MYC(4609); |
| | | LZTS1(11178); MTSS1(9788); FBXL6(26233); JAK2(3717); C9orf89(84270); |
| | | FREQ(23413); ABL1(25); PTCH(5727); TRAF1(7185); MAPKAP1 |
| | | (79109); CARD9(64170); LZTS2(84445); CSPG6(9126); DCLRE1A(9937); |
| | | WEE1(7465); LRP5(4041); IGF2(3481); HRASLS3(11145); CDCA5(113130); |
| | | MTL5(9633); PHOX2A(401); CASP5(838); BCL9L(283149); ESPL1(9700); |
| | | MDM2(4193); GAS2L3(283431); KNTC1(9735); FAIM2(23017); SCARB1 |
| | | (949); RFP2(10206); PNN(5411); PPP2R5C(5527); TRAF3(7187); CHES1 |
| | | (1112); JAG2(3714); ISLR2(57611); IGF1R(3480); HERC2(8924); TSC2 |
| | | (7249); KATNB1(10300); ELMO3(79767); NME3(4832); ERN2(10595); TRADD |
| | | (8717); PCOLN3(5119); MAPT(4137); TTYH2(94015); FLJ35220(284131); |
| | | SLC25A10(1468); P2RX1(5023); SPAG5(10615); ERN1(2081); REEP6 |
| | | (92840); FZR1(51343); JMJD2B(23030); PIK3R2(5296); FOSB(2354); |
| | | ATF5(22809); LENG8(114823); TIMM13(26517); DAPK3(1613); PLAC2 |
| | | (257000); DPF1(8193); STK4(6789); DGCR8(54487); NF2(4771); PSCD4(27128); |
| | | MAPK8IP2(23542); MN1(4330); TTLL12(23170); PCTK1(5127); MLLT7 |
| | | (4303); ARHGEF6(9459); HCFC1(3054); |
| hsa-miR-614 | 2092; | SAMD11(148398); GABRD(2563); KIF1B(23095); C1orf187(374946); FAM76A |
| | | (199870); RBBP4(5928); C1orf113(79729); C1orf83(127428); RAVER2 |
| | | (55225); GADD45A(1647); RAP1A(5906); SOAT1(6646); C1orf21(81563); |
| | | IL24(11009); C1orf69(200205); GNPAT(8443); CHD5(26038); ACOT7(11332); |
| | | MTHFR(4524); THEM5(284486); GBA(2629); TOMM20(9804); RHOB |
| | | (388); FLJ30851(375190); EML4(27436); SOCS5(9655); EPB41L5(57669); |
| | | COL4A3(1285); LOC129607(129607); C2orf32(25927); ANKRD23(200539); |
| | | FLJ20297(55627); RND3(390); C2orf19(394261); CLDN11(5010); ZNF445 |
| | | (353274); FYCO1(79443); VPRBP(9730); C3orf63(23272); DNASE1L3 |
| | | (1776); KPNA1(3836); C3orf33(285315); MUC4(4585); HD(3064); EVC |
| | | (2121); MED28(80306); GENX-3414(8987); CCRN4L(25819); STOX2(56977); |
| | | MRFAP1L1(114932); ABLIM2(84448); SRD5A2L2(253017); CXXC4 |
| | | (80319); TIFA(92610); BBS7(55212); GYPA(2993); NEK1(4750); FLJ11200 |
| | | (55325); GRAMD3(65983); PURA(5813); GRPEL2(134266); LSM11(134353); |
| | | CPEB4(80315); ZNF346(23567); LOC134145(134145); LOC153561 |
| | | (153561); FOXD1(2297); ATG12(9140); NRG2(9542); CAMK2A(815); RPS14 |
| | | (6208); CDYL(9425); LRRC16(55604); ZNF192(7745); C6orf205(394263); |
| | | DAAM2(23500); C6orf153(88745); KIAA1586(57691); TCF21(6943); SASH1 |
| | | (23328); TXNDC5(81567); NEDD9(4739); DCDC2(51473); DHX16( |

Table 4-126

| | | |
|---|---|---|
| | | 8449); LTB(4050); C6orf125(84300); BACH2(60468); PBOV1(59351); FBXO30 |
| | | (84085); PIP3-E(26034); AGPAT4(56895); PARK2(5071); PDE10A(10846); |
| | | AHR(196); WBSCR1(7458); ZFP95(23660); KIAA0265(23008); LOC155060 |
| | | (155060); GALNT11(63917); SNX8(29886); CYCS(54205); HOXA3 |
| | | (3200); YWHAG(7532); FLJ13576(64418); ZNF425(155054); DLGAP2(9228); |
| | | MYOM2(9172); KIAA1456(57604); EXTL3(2137); EIF4EBP1(1978); |
| | | XKR5(389610); PPP1R3B(79660); CLU(1191); RDHE2(195814); KLF10 |
| | | (7071); SECISBP2(79048); ZNF618(114991); LRRC8A(56262); FREQ(23413); |
| | | C9orf48(347240); ZCCHC6(79670); C9orf10OS(158293); TXNDC4(23071); |
| | | UCK1(83549); REXO4(57109); LOC399706(399706); SGPL1(8879); |
| | | RPP30(10556); INA(9118); ADD3(120); LRRC27(80313); MTG1(92170); SFMBT2 |
| | | (57713); MARCH8(220972); HTR7(3363); ARHGAP19(84986); PSMD13 |
| | | (5719); GPIAP1(4076); TSPAN18(90139); SCGB2A1(4246); RARRES3 |
| | | (5920); PACS1(55690); BBS1(582); BIRC2(329); NCAM1(4684); CEP164 |
| | | (22897); RNF26(79102); C11orf37(440072); MUCDHL(53841); IGF2(3481); |
| | | PLEKHA7(144100); MUC15(143662); SYT7(9066); CDCA5(113130); |
| | | SUV420H1(51111); NDUFC2(4718); FZD4(8322); GRM5(2915); FLJ45803 |
| | | (399948); TMPRSS13(84000); hCAP-D3(23310); CACNA1C(775); COPS7A |
| | | (50813); A2ML1(144568); APOLD1(81575); DDX11(1663); PA2G4(5036); |
| | | MDM2(4193); UTP20(27340); ACACB(32); OAS2(4939); TMEM132B |
| | | (114795); P2RX2(22953); BHLHB3(79365); VDR(7421); DIP13B(55198); GLTP |
| | | (51228); C12orf43(64897); GPR109B(8843); ZNF605(90462); DZIP1 |
| | | (22873); C14orf21(161424); PNN(5411); SMOC1(64093); WDR21A(26094); |
| | | VASH1(22846); CALM1(801); C14orf159(80017); TCL6(27004); EML1(2009); |
| | | KIAA0329(9895); VTI1B(10490); ABCD4(5826); BCL11B(64919); KLF13 |
| | | (51621); IL16(3603); SLC28A1(9154); MAN2A2(4122); SLCO3A1(28232); |
| | | TTBK2(146057); HEXA(3073); ACSBG1(23205); RHOT2(89941); C16orf34 |
| | | (90861); CCNF(899); PMM2(5373); MGC35048(124152); PRRT2 |
| | | (112476); DDX19-DDX19L(544314); HSBP1(3281); FBKL16(146330); ZNF597 |
| | | (146434); C16orf5(29965); KIAA1970(124454); CBFA2T3(863); GSG2 |
| | | (83903); SMCR7(125170); RHOT1(55288); PPP1R1B(84152); ADAM11 |
| | | (4185); WDR68(10238); INCA1(388324); PITPNM3(83394); PER1(5187); |
| | | GAS7(8522); IFT20(90410); CORO6(84940); TBC1D3C(414060); NEUROD2 |
| | | (4761); HDAC5(10014); TRIM25(7706); CUEDC1(404093); PTRH2(51651); |
| | | GNA13(10672); GRB2(2885); EXOC7(23265); MAFG(4097); CEP192 |
| | | (55125); CABLES1(91768); MBD1(4152); TCF4(6925); ZADH2(284273); |
| | | UHRF1(29128); EMR1(2015); ZNF317(57693); ANKRD41(126549); LOC93343 |
| | | (93343); ARRDC2(27106); NFKBIB(4793); TEX101(83639); ZNF224 |
| | | (7767); VASP(7408); TSEN34(79042); NALP8(126205); FLJ45684(400666); |
| | | AP3D1(8943); GNG7(2788); SGTA(6449); AES(166); PIP5K1C(23396); |
| | | HOOK2(29911); AKAP8(10270); SF4(57794); RTN2(6253); FLJ10781(55228); |
| | | KLK14(43847); LOC352909(352909); PLCB1(23236); PROCR(10544); |
| | | DNAJC5(80331); SCRT2(85508); RP5-1022P6.2(56261); RP5-1054A22.3 |
| | | (85449); NFATC2(4773); DIDO1(11083); C21orf33(8209); FLJ33814 |
| | | (150275); NF2(4771); LOC400924(400924); SYNGR1(9145); NUP50(10762); |
| | | FBLN1(2192); CLDN5(7122); C22orf9(23313); CHKB(1120); SHOX(6473); |
| | | CD99(4267); XG(7499); MID1IP1(58526); NYX(60506); JM11(90060); |
| | | PCDH11X(27328); GPRASP2(114928); DUSP9(1852); BRCC3(79184); SPRY3 |
| | | (10251); GTPBP6(8225); |
| hsa-miR-615 | 2093; 2321; | PRKCZ(5590); PAX7(5081); SFN(2810); SESN2(83667); RCC1(1104); MACF1 |
| | | (23499); CDKN2C(1031); PEA15(8682); UCK2(7371); MDM4(4194); |
| | | CHD5(26038); CTNNBIP1(56998); CLSPN(63967); GFI1(2672); NOTCH2 |
| | | (4853); PYGO2(90780); GLRX2(51022); ASPM(259266); PTPN7(5778); PRKCE |
| | | (5581); CCT7(10574); ALS2CR19(117583); BOK(666); MCM6(4175); |
| | | TLK1(9874); FRZB(2487); STAT1(6772); FZD5(7855); PAX3(5077); HDAC4 |
| | | (9759); PCAF(8850); CAMK1(8536); CIDEC(63924); VGLL4(9686); TRAIP |
| | | (10293); ANAPC4(29945); FGF2(2247); LRPAP1(4043); SFRP2(6423); |
| | | CEI(153571); RAD17(5884); APC(324); CSNK1G3(1456); UNC5A(90249); |
| | | UNC2(10309); NRG2(9542); FBXW11(23291); RNF130(55819); MAPK13 |
| | | (5603); PARC(23113); PTP4A1(7803); FOXO3A(2309); HECA(51696); SASH1 |
| | | (23328); MDC1(9656); FRK(2444); SGK(6446); STK17A(9263); FZD9 |
| | | (8326); STAG3(10734); MICAL-L2(79778); MAD1L1(8379); FIS1(51024); |
| | | PODXL(5420); FASTK(10922); GLI4(2738); ADRA1A(148); SFRP1(6422); |
| | | MOS(4342); STK3(6788); CDC37L1(55664); RECK(8434); INVS(27130); |
| | | SPTAN1(6709); BAG1(573); GAS1(2619); BARX1(56033); CIZ1(25792); A |

Table 4-127

| | | |
|---|---|---|
| | | NAPC2(29882); CUGBP2(10659); PTEN(5728); LZTS2(84445); PDCD11 |
| | | (22984); DOCK1(1793); FBXW4(6468); BRSK2(9024); WEE1(7465); F2(2147); |
| | | CAPN1(823); SIPA1(6494); CDKN1C(1028); LTBP3(4054); FGF4(2249); |
| | | LTBR(4055); PTPN6(5777); ESPL1(9700); PA2G4(5036); TCF1(6927); |
| | | FZD10(11211); BCAT1(586); KRAS(3845); HDAC7A(51564); FAIM2(23017); |
| | | C12orf22(81566); DUSP6(1848); DIP13B(55198); SCARB1(949); ING1 |
| | | (3621); CUL4A(8451); PNN(5411); TRAF3(7187); STRN3(29966); EGLN3 |
| | | (112399); NFKBIA(4792); DLG7(9787); LTBP2(4053); PGF(5228); AKT1 |
| | | (207); CCNB2(9133); IGF1R(3480); RAB40C(57799); KIAA1924(197335); |
| | | KREMEN2(79412); TNFRSF12A(51330); PLK1(5347); CARD15(64127); |
| | | GNAO1(2775); FLJ21736(79984); NOL3(8996); E2F4(1874); AXIN1(8312); |
| | | APPBP1(8883); TRADD(8717); SMPD3(55512); TERF2(7014); MAPK7 |
| | | (5598); TRAF4(9618); MAPT(4137); NGFR(4804); CARD14(79092); NALP1 |
| | | (22861); AURKB(9212); GAS7(8522); ERN1(2081); CBX4(8535); MALT1 |
| | | (10892); CDC34(997); GADD45B(4616); NCLN(56926); FZR1(51343); ALKBH7 |
| | | (84266); CRB3(92359); TNFSF9(8744); DNM2(1785); F2RL3(9002); |
| | | APLP1(333); ZFP36(7538); AXL(558); LOC400707(400707); SPHK2(56848); |
| | | PPP2R1A(5518); NALP2(55655); AURKC(6795); MOBKL2A(126308); |
| | | MATK(4145); CDC37(11140); YIPF2(78992); LPHN1(22859); DPF1(8193); |
| | | PRX(57716); TRIB3(57761); CDC25B(994); KCNK15(60598); PARD6B |
| | | (84612); SCAND1(51282); UBE2V1(7335); ZBP1(81030); DIDO1(11083); |
| | | MX1(4599); APP(351); SEPT5(5413); GAS2L1(10634); BID(637); PPM1F |
| | | (9647); MN1(4330); CARD10(29775); PDGFB(5155); TOB2(10766); MAPK12 |
| | | (6300); BIRC4(331); RP11-308D16.4(441522); ARHGAP6(395); SMC1L1 |
| | | (8243); PDCD8(9131); |
| hsa-miR-616 | 2094; 2130; | H6PD(9563); CDC42(998); HDAC1(3065); RBBP4(5928); CDC20(991); PRKACB |
| | 2131; | (5567); SH3GLB1(51100); ZNF326(284695); CDC7(8317); TMEM56 |
| | | (148534); RAP1A(5906); SYCP1(6847); BNIPL(149428); UHMK1(127933); |
| | | DDR2(4921); MPZL1(9019); FASLG(356); RALGPS2(55103); FAM20B |
| | | (9917); NR5A2(2494); MDM4(4194); IL24(11009); TRAF5(7188); TGFB2 |
| | | (7042); FBXO28(23219); C1orf69(200205); DISC1(27185); CIAS1(114548); |
| | | CCNL2(81669); TNFRSF9(3604); KIAA0090(23065); E2F2(1870); ZBTB8OS |
| | | (339487); CLSPN(63967); SERBP1(26135); BCL10(8915); NRAS(4893); |
| | | NOTCH2(4853); TNFSF18(8995); ASPM(259266); SLC30A1(7779); ENAH |
| | | (55740); C1orf96(126731); GNG4(2786); FH(2271); PQLC3(130814); MAPRE3 |
| | | (22924); PPP1CB(5500); BIRC6(57448); PLEKHH2(130271); HTLF |
| | | (3344); C2orf13(200558); HK2(3099); MRPL19(9801); MGC5509(79074); |
| | | RIF1(55183); TAIP-2(80034); ZAK(51776); DUSP19(142679); FLJ38973 |
| | | (205327); CASP10(843); CASP8(841); FZD7(8324); CD28(940); ALS2CR19 |
| | | (117583); CREB1(1385); RPE(6120); PSMD1(5707); SEPT2(4735); CGREF1 |
| | | (10669); SERTAD2(9792); TIA1(7072); AFF3(3899); ST6GAL2(84620); |
| | | SEPT10(151011); BUB1(699); IL1B(3553); TLK1(9874); SESTD1(91404); |
| | | FRZB(2487); NCKAP1(10787); STAT1(6772); PAX3(5077); CUL3(8452); |
| | | HDAC4(9759); FANCD2(2177); VHL(7428); MLH1(4292); TDGF1(6997); |
| | | APPL(26060); ATXN7(6314); COL8A1(1295); DTX3L(151636); UBE1DC1 |
| | | (79876); RNF7(9616); PIK3CA(5290); SENP2(59343); SENP5(205564); TADA3L |
| | | (10474); XPC(7508); MAP4(4134); NEK4(6787); CAST1(26059); VGLL3 |
| | | (389136); WDR5B(54554); STAG1(10274); KPNA4(3840); ELF5A2 |
| | | (56648); TNFSF10(8743); MCF2L2(23101); HD(3064); ANAPC4(29945); PCDH7 |
| | | (5099); TMEM33(55161); LOC401137(401137); MTHFD2L(441024); |
| | | EREG(2069); FGF5(2250); SYNPO2(171024); PLK4(10733); ARFIP1(27236); |
| | | GAK(2580); C4orf15(79441); FLJ38991(285521); CCNI(10983); SCD5 |
| | | (79966); SH3D19(152503); SFRP2(6423); NEK1(4750); SKP2(6502); MRPS30 |
| | | (10884); ERBB2IP(55914); SCAMP1(9522); CAMK4(814); APC(324); |
| | | DCP2(167227); LOC133619(133619); RAD50(10111); TCF7(6932); KIAA0141 |
| | | (9812); SGCD(6444); PRLR(5618); PRKAA1(5562); ARSB(411); MTX3 |
| | | (345778); ST8SIA4(7903); MCC(4163); ATG12(9140); SEMA6A(57556); IRF1 |
| | | (3659); HDAC3(8841); CSNK1A1(1452); FBXW11(23291); DUSP1(1843); |
| | | ADAMTS2(9509); RNF130(55819); IRF4(3662); HIST1H3E(8353); BRD2 |
| | | (6046); CDKN1A(1026); PARC(23113); POLR1C(9533); PTP4A1(7803); |
| | | TTK(7272); ANKRD6(22881); FOXO3A(2309); TBPL1(9519); HECA(51696); |
| | | SASH1(23328); TULP4(56995); RP11-145H9.1(340156); SERPINB9 |
| | | (5272); NEDD9(4739); MDC1(9656); CUL7(9820); TRAM2(9697); DST |
| | | (667); CCNC(892); SESN1(27244); CDC2L6(23097); STX7(8417); BCLAF1 |
| | | (9774); MAP3K5(4217); PLAGL1(5325); LATS1(9113); PIP3-E(26034); PD |

Table 4-128

CD2(5134); FOXK1(221937); AHR(196); HDAC9(9734); SEPT7(989); STK17A
(9263); EGFR(1956); PHTF2(57157); CLDN12(9069); FZD1(8321); ZKSCAN1
(7586); RINT1(60561); BCAP29(55973); AKR1D1(6718); CASP2(835);
CYCS(54205); TMED4(222068); PURB(5814); IGFBP3(3486); CDK6
(1021); CLEC5A(23601); BNIP3L(665); LEPROTL1(23484); BAG4(9530);
TACC1(6867); UBE2V2(7336); RIPK2(8767); MTDH(92140); SLC30A8(169026);
PHF20L1(51105); WISP1(8840); TNFRSF10B(8795); TNFRSF10D
(8793); ADRA1A(148); CCDC25(55246); WHSC1L1(54904); IMPAD1(54928);
VCPIP1(80124); TP53INP1(94241); STK3(6788); RAD21(5885); EXT1
(2131); ANKRD15(23189); JAK2(3717); C9orf47(286223); INVS(27130);
PAPPA(5069); OLFML2A(169611); TRAF2(7186); PSIP1(11168); KLHL9
(55958); BAG1(573); SEMA4D(10507); PTCH(5727); TNFSF8(944); ZBTB26
(57684); TSC1(7248); CARD9(64170); ZMYND11(10771); CUGBP2
(10659); ZNF33A(7581); ZNF22(7570); ARID5B(84159); REEP3(221035);
EIF4EBP2(1979); SGPL1(8879); KIAA1128(54462); PTEN(5728); FAS(355);
RPP30(10556); KIF11(3832); CCNJ(54619); SCD(6319); BTRC(8945);
NOLC1(9221); PDCD11(22984); DOCK1(1793); PAPD1(55149); PARD3(56288);
CUL2(8453); C10orf72(196740); MRPS16(51021); COX15(1355); SMNDC1
(10285); C10orf46(143384); MKI67(4288); STIM1(6786); RPL27A
(6157); HIPK3(10114); API5(8539); RPS6KB2(6199); LRRC51(220074);
PANX1(24145); CUL5(8065); ATM(472); CBL(867); LOH11CR2A(4013); EI24
(9538); HRAS(3265); EIF4G2(1982); DKK3(27122); WT1(7490); SLC1A2
(6506); ZBTB3(79842); HBXAP(51773); FZD4(8322); GRM5(2915); SESN3
(143686); C12orf32(83695); CCND2(894); CDKN1B(1027); ATF7IP(55729);
FGD4(121512); TEGT(7009); PDE1B(5153); CDK2(1017); RBMS2
(5939); SLC16A7(9194); MDM2(4193); KCNMB4(27345); KIAA0152(9761);
KNTC1(9735); RAD52(5893); NOL1(4839); KRAS(3845); TMTC1(83857);
ZNF641(121274); CBX5(23468); CDK4(1019); GNS(2799); PHLDA1(22822);
EEA1(8411); PCTK2(5128); PPP1CC(5501); MPHOSPH9(10198); CDK2AP1
(8099); SBNO1(55206); GOLGA3(2802); CDK8(1024); BRCA2(675);
RFP2(10206); SUGT1(10910); ING1(3621); CUL4A(8451); CSNK1A1L
(122011); FOXO1A(2308); KCTD12(115207); DZIP1(22873); BCL2L2(599);
ARHGAP5(394); PNN(5411); C14orf111(51077); FLRT2(23768); TRAF3
(7187); CDKL1(8814); DLG7(9787); EXOC5(10640); LTBP2(4053); NEK9
(91754); BAG5(9529); AKT1(207); BUB1B(701); EIF3S1(8669); FGF7(2252);
MAPK6(5597); Gcom1(145781); CCNB2(9133); RAB11A(8766); CSK
(1445); RCCD1(91433); IGF1R(3480); BMF(90427); TP53BP1(7158); PRTG
(283659); CSNK1G1(53944); PDCD7(10081); AP3S2(10239); C15orf38
(348110); LINS1(55180); DNASE1(1773); DNAJA3(9093); PLK1(5347); SPN
(6693); PAPD5(64282); CYLD(1540); CTCF(10664); HAS3(3038); CDT1
(81620); GSPT1(2935); FLJ32130(146540); SIAH1(6477); FTS(64400); RANBP10
(57610); TERF2(7014); EXOSC6(118460); TXNL4B(54957); ADAMTS18
(170692); PAFAH1B1(5048); MAPK7(5598); NLK(51701); NF1(4763);
CCL2(6347); TADA2L(6871); CDC6(990); G6PC(2538); CROP(51747);
NME1(4830); PPM1D(8493); PRKCA(5578); MAP2K6(5608); MNT(4335);
NALP1(22861); TP53(7157); GAS7(8522); FAM106A(80039); TIAF1(9220);
NUFIP2(57532); LOC162427(162427); DUSP3(1845); SPAG9(9043); F
AM104A(84923); SUMO2(6613); ACOX1(51); CETN1(1068); VAPA(9218);
HRH4(59340); SMAD4(4089); COLEC12(81035); ROCK1(6093); SYT4(6860);
SMAD2(4087); MBD2(8932); BCL2(596); ZADH2(284273); PARD6G
(84552); FZR1(51343); TNFSF9(8744); DNM2(1785); PIK3R2(5296); UBA52
(7311); CCNE1(898); ARHGEF1(9138); APOE(348); LOC390980(390980);
ZNF587(84914); DAPK3(1613); ZNF490(57474); ZNF100(163227); KIAA1559
(57677); TGFB1(7040); LIG1(3978); CARD8(22900); MGC35402
(399669); ZNF702(79986); MCM8(84515); PLCB1(23236); KIAA1219(57148);
STK4(6789); SLC2A10(81031); CSE1L(1434); PARD6B(84612); C20orf177
(63939); CSNK2A1(1457); RBL1(5933); AURKA(6790); SYCP2(10388);
DIDO1(11083); ABCC13(150000); MX1(4599); D21S2056E(8568); NF2
(4771); APOL6(80830); APOBEC3F(200316); MAPK1(5594); ZNF70(7621);
SMC1L2(27127); PDHA1(5160); EIF2S3(1968); USP9X(8239); PCTK1
(5127); ZC3H12B(340554); dJ341D10.1(286453); ATP1B4(23439); BIRC4
(331); TRAPPC2(6399); FIGF(2277); KLHL15(80311); SMC1L1(8243);
WNK3(65267); OPHN1(4983); RNF12(51132); TAF9B(51616); SEPT6(23157);
LAMP2(3920); CUL4B(8450); ARHGEF6(9459); CETN2(1069);

Table 4-129

| | | |
|---|---|---|
| hsa-miR-617 | 2095; | NBL1(4681); SESN2(83667); KHDRBS1(10657); RBBP4(5928); MACF1 |
| | | (23499); RP4-692D3.1(80098); CDC14A(8556); CYB561D1(284613); RAP1A |
| | | (5906); BCL9(607); DAP3(7818); PEA15(8682); IPO9(55705); TGFB2(7042); |
| | | ACTN2(88); EGFL3(1953); KLHL21(9903); TNFRSF9(3604); ZBTB17 |
| | | (7709); RCC2(55920); E2F2(1870); EPHA10(284656); MCL1(4170); JTB |
| | | (10899); SHE(126669); PYGO2(90780); CD5L(922); DEDD(9191); ASPM(259266); |
| | | PLEKHA6(22874); TP53BP2(7159); SOX11(6664); PRKCE(5581); |
| | | GKN1(56287); HTRA2(27429); HK2(3099); MAL(4118); TTL(150465); CASP10 |
| | | (843); CASP8(841); FZD7(8324); CD28(940); COL4A3(1285); BOK |
| | | (666); CARD12(58484); RTN4(57142); RetSat(54884); EDAR(10913); ANAPC1 |
| | | (64682); BIN1(274); ERCC3(2071); FLJ13984(79828); TATDN2(9797); |
| | | DLEC1(9940); TDGF1(6997); C3orf37(56941); SENP2(59343); NT5DC2 |
| | | (64943); STAG1(10274); MGC21675(92070); HD(3064); CD38(952); HIP2 |
| | | (3093); SEPT11(55752); FGF2(2247); PLK4(10733); APBB2(323); UNC5C |
| | | (8633); DKK2(27123); MAD2L1(4085); NIPBL(25836); CCNB1(891); APC |
| | | (324); SGCD(6444); ERGIC1(57222); SEMA5A(9037); PRLR(5618); PRKAA1 |
| | | (5562); MCC(4163); SPOCK1(6695); E2F3(1871); PACSIN1(29993); PTP4A1 |
| | | (7803); TCF21(6943); TNFAIP3(7128); SASH1(23328); MDC1(9656); |
| | | C6orf47(57827); CCND3(896); SUPT3H(8464); TNFRSF21(27242); TRAM2 |
| | | (9697); DST(667); CCNC(892); FRK(2444); BCLAF1(9774); PDCD2(5134); |
| | | AHR(196); HDAC9(9734); RINT1(60561); DLD(1738); KIAA0265(23008); |
| | | CASP2(835); PMS2(5395); KDELR2(11014); CARD4(10392); ELMO1 |
| | | (9844); ECOP(81552); MGC5242(78996); PTN(5764); ZC3HAV1(56829); BRAF |
| | | (673); SLC39A14(23516); TNFRSF10C(8794); BNIP3L(665); UNC5D |
| | | (137970); ZHX2(22882); WISP1(8840); TNFRSF10B(8795); TNFRSF10A |
| | | (8797); ADRA1A(148); JPH1(56704); RUNX1T1(862); CCNE2(9134); LYNX1 |
| | | (66004); ANKRD15(23189); CDC37L1(55664); MRRF(92399); CRB2 |
| | | (286204); C9orf28(89853); STXBP1(6812); PTCH(5727); CTNNAL1(8727); |
| | | EPB41L4B(54566); TNFSF15(9966); FNBP1(23048); TSC1(7248); ANAPC2 |
| | | (29882); C10orf9(219771); SIRT1(23411); SGPL1(8879); PTEN(5728); |
| | | CSPG6(9126); DOCK1(1793); DLG5(9231); SFRP5(6425); MRPL43(84545); |
| | | VAX1(11023); C10orf46(143384); MKI67(4288); STIM1(6786); HIPK3 |
| | | (10114); SIPA1(6494); LRP5(4041); ABCG4(64137); TBRG1(84897); IGF2 |
| | | (3481); CLCF1(23529); FZD4(8322); CCND2(894); CNAP1(9918); PTMS(5763); |
| | | KIAA1467(57613); PDE3A(5139); DIP2B(57609); ACVR1B(91); ESPL1 |
| | | (9700); CCT2(10576); APAF1(317); TNFRSF1A(7132); RERG(85004); |
| | | CDK4(1019); LRRC10(376132); BTG1(694); PCTK2(5128); BRCA2(675); |
| | | HS6ST3(266722); C13orf6(84945); CSNK1A1L(122011); UGCGL2(55757); |
| | | PNN(5411); TRAPPC6B(122553); CDKL1(8814); NEK9(91754); TGFB3 |
| | | (7043); WARS(7453); BAG5(9529); AKT1(207); GREM1(26585); CCNB2 |
| | | (9133); SMAD3(4088); NEO1(4756); BMF(90427); CSNK1G1(53944); TSC2 |
| | | (7249); CCNF(899); PRRT2(112476); PAPD5(64282); CARD15(64127); RBL2 |
| | | (5934); SNTB2(6645); ERN2(10595); STX1B2(112755); TK2(7084); MPHOSPH6 |
| | | (10200); PCOLN3(5119); PRDM7(11105); BIRC4BP(54739); TNFSF12 |
| | | (8742); MAPK7(5598); CCL4L1(9560); MAPT(4137); NGFR(4804); |
| | | TLK2(11011); PRKCA(5578); PITPNA(5306); P2RX1(5023); NALP1(22861); |
| | | LOC201164(201164); FAM106A(80039); SPAG5(10615); GAS2L2(246176); |
| | | CCL5(6352); BRCA1(672); HDAC5(10014); FLJ45079(400624); C17orf55 |
| | | (284185); CEP192(55125); CABLES1(91768); MAPK4(5596); DCC |
| | | (1630); SOCS6(9306); TXNL1(9352); BCL2(596); PARD6G(84552); CHAF1A |
| | | (10036); UHRF1(29128); ALKBH7(84266); DNM2(1785); SYDE1(85360); |
| | | CCNE1(898); ZNF480(147657); LOC390980(390980); TNFSF14(8740); |
| | | FLJ12949(65095); ZNF443(10224); PPP1R13L(10848); LIG1(3978); ITCH |
| | | (83737); MYBL2(4605); SLC2A10(81031); PARD6B(84612); C20orf11 |
| | | (54994); ZGPAT(84619); SYCP2(10388); ABCC13(150000); SNF1LK(150094); |
| | | PTTG1IP(754); SEPT5(5413); GAS2L1(10634); NF2(4771); PGEA1(25776); |
| | | TNRC6B(23112); GTSE1(51512); hCAP-H2(29781); BID(637); PPM1F |
| | | (9647); MN1(4330); SSTR3(6753); CARD10(29775); CCNB3(85417); IKBKG |
| | | (8517); DKC1(1736); BTK(695); PDCD8(9131); ARHGEF6(9459); |
| | | MTCP1(4515); |
| hsa-miR-618 | 2096; | ROC1(1104); GIPC2(54810); CDC7(8317); SYCP1(6847); C1orf56(54964); |
| | | ATF6(22926); UHMK1(127933); FMO2(2327); LAMC2(3918); C1orf21(81563); |
| | | TGFB2(7042); MARK1(4139); RHOU(58480); CCNL2(81669); DFFA |
| | | (1676); E2F2(1870); SF3A3(10946); RBMXL1(494115); TMED5(50999); TRIM33 |
| | | (51592); MCL1(4170); DEDD(9191); ASPM(259266); ZBTB41(360 |

Table 4-130

| | | |
|---|---|---|
| | | 023); TP53BP2(7159); SELI(85465); PPP1CB(5500); DYNC2LI1(51626); |
| | | ABCG8(64241); MSH2(4436); HK2(3099); RIF1(55183); PPIG(9360); ZAK |
| | | (51776); CFLAR(8837); ABI2(10152); ANKZF1(55139); ROCK2(9475); KIAA1212 |
| | | (55704); CCT4(10575); TGFA(7039); EDAR(10913); ANAPC1(64682); |
| | | TLK1(9874); WASPIP(7456); SESTD1(91404); STAT1(6772); STK17B |
| | | (9262); RAPH1(65059); LANCL1(10314); PAX3(5077); CUL3(8452); HDAC4 |
| | | (9759); EDEM1(9695); FANCD2(2177); CTNNB1(1499); ZNF662(389114); |
| | | ZNF660(285349); TDGF1(6997); APPL(26060); ACPP(55); KLHL24(54800); |
| | | SENP2(59343); ZFYVE20(64145); VGLL3(389136); CBLB(868); STAG1 |
| | | (10274); SIAH2(6478); B3GALT3(8706); KLHL6(89857); HD(3064); |
| | | HCAP-G(64151); KLF3(51274); FGF2(2247); GAB1(2549); TRIM2(23321); |
| | | TLR2(7097); PPBP(5473); DKK2(27123); CASP6(839); ASAP(79884); NEK1 |
| | | (4750); RAD17(5884); F2R(2149); LOC91137(91137); DCP2(167227); |
| | | CSNK1G3(1456); RAD50(10111); FLJ37562(134553); SEMA5A(9037); FLJ21657 |
| | | (64417); HCN1(348980); ELOVL7(79993); BIRC1(4671); MCC(4163); |
| | | PPP2CA(5515); CDC23(8697); RIPK1(8737); E2F3(1871); CDC5L(988); |
| | | IL17(3605); LRRC1(55227); RNF146(81847); RP5-875H10.1(389432); |
| | | TXNDC5(81567); NEDD9(4739); TAP2(6891); DST(667); SYNCRIP(10492); |
| | | BCLAF1(9774); PLAGL1(5325); PDCD2(5134); GLCCI1(113263); HDAC9 |
| | | (9734); SEPT7(989); STK17A(9263); LOC441257(441257); STEAP2 |
| | | (261729); ING3(54556); KIAA0265(23008); CNTNAP2(26047); MMD2(221938); |
| | | DRCTNNB1A(84668); CYCS(54205); VPS41(27072); INHBA(3624); |
| | | GRB10(2887); LUZP5(54892); MTMR9(66036); ATP6V1B2(526); BAG4 |
| | | (9530); TACC1(6867); HNF4G(3174); FZD6(8323); TNFRSF10D(8793); LSM1 |
| | | (27257); TCEB1(6921); TPD52(7163); CCNE2(9134); MGC39715(169166); |
| | | TNFRSF11B(4982); RECK(8434); PTPDC1(138639); PALM2(114299); |
| | | KGFLP2(654466); KLF9(687); SEMA4D(10507); TXNDC4(23071); MRPL50 |
| | | (54534); C9orf125(84302); C9orf5(23731); TNFSF15(9966); PRKG1 |
| | | (5592); KIAA1128(54462); PTEN(5728); CYP2C9(1559); DLG5(9231); RAB11FIP2 |
| | | (22841); MKI67(4288); OLFML1(283298); RAG1(5896); API5(8539); |
| | | BIRC2(329); CUL5(8065); ATM(472); CHEK1(1111); HNT(50863); ZNF195 |
| | | (7748); EIF4G2(1982); ELF5(2001); CDCA5(113130); HBXAP(51773); |
| | | RAB30(27314); MDS025(60492); FZD4(8322); JOSD3(79101); SESN3(143686); |
| | | PPP2R1B(5519); SRPR(6734); CDK2(1017); GAS2L3(283431); RAD9B |
| | | (144715); BCAT1(586); TMTC1(83857); ZNF641(121274); C12orf22 |
| | | (81566); ATF7(11016); CBX5(23468); E2F7(144455); SLC41A2(84102); FGF9 |
| | | (2254); AKAP11(11215); UBL3(5412); KCTD12(115207); LIG4(3981); FRMD6 |
| | | (122786); CHES1(1112); BAG5(9529); GOLGA8E(390535); GOLGA8G |
| | | (283768); THBS1(7057); FGF7(2252); CCNB2(9133); SMAD3(4088); |
| | | CSK(1445); LOC440295(440295); UBE3A(7337); ARPP-19(10776); PRTG |
| | | (283659); NEDD4(4734); RPL4(6124); ABAT(18); CTCF(10664); PDPR(55066); |
| | | HSBP1(3281); GRIN2A(2903); PRDM7(11105); KSR1(8844); RAB11FIP4 |
| | | (84440); ZNF207(7756); TLK2(11011); BAIAP2(10458); NCOR1(9611); |
| | | SSH2(85464); PLXDC1(57125); PPARBP(5469); TRIM25(7706); HELZ |
| | | (9931); PSMD12(5718); RNMT(8731); STATIP1(55250); ENOSF1(55556); |
| | | ESCO1(114799); DSC2(1824); FAM59A(64762); PSTPIP2(9050); MBD2(8932); |
| | | RNF152(220441); BCL2(596); ZNF431(170959); ZNF302(55900); SIPA1L3 |
| | | (23094); AXL(558); APOE(348); SPHK2(56848); SLC39A3(29985); |
| | | PRDX2(7001); MAPRE1(22919); CEP250(11190); STK4(6789); UBE2C(11065); |
| | | PARD6B(84612); STX16(8675); C20orf6(51575); ABCC13(150000); |
| | | SON(6651); APP(351); SNAP29(9342); ST13(6767); DNAJB7(150353); TOB2 |
| | | (10766); XK(7504); CLCN5(1184); MLLT7(4303); ZNF6(7552); BIRC4 |
| | | (331); ARSD(414); RPS6KA3(6197); SEPT6(23157); LAMP2(3920); CUL4B |
| | | (8450); ATP11C(286410); MECP2(4204); |
| hsa-miR-621 | 2097; | RBBP4(5928); CDC14A(8556); PBX1(5087); RGS2(5997); PKP1(5317); TNFRSF14 |
| | | (8764); C1orf183(55924); TP53BP2(7159); PPP1CB(5500); BIRC6 |
| | | (57448); PSMD1(5707); FAM49A(81553); CGREF1(10669); IFIH1(64135); |
| | | STK17B(9262); CUL3(8452); HDAC4(9759); PDCD6IP(10015); SEC61A1 |
| | | (29927); NEK11(79858); SKIL(6498); NGLY1(55768); RASSF1(11186); |
| | | ATR(545); TNFSF10(8743); SH3BP2(6452); HD(3064); IL8(3576); FLJ20032 |
| | | (54790); SNCA(6622); LEF1(51176); RAD17(5884); APC(324); OSRF |
| | | (23548); BIRC1(4671); FLJ35779(134359); SEMA6A(57556); CD14(929); |
| | | CSNK1A1(1452); E2F3(1871); PARC(23113); VEGF(7422); FOXO3A(2309); |
| | | SASH1(23328); CUL7(9820); DST(667); BCLAF1(9774); BCAP29(55973); |
| | | CASP2(835); DRCTNNB1A(84668); CDK6(1021); ZC3HC1(51530); SL |

Table 4-131

| | | |
|---|---|---|
| | | C7A2(6542); TACC1(6867); DUSP4(1846); ANKRD15(23189); JAK2(3717); |
| | | NTRK2(4915); C9orf28(89853); CDKN2A(1029); PPP3R2(5535); SIRT1 |
| | | (23411); HECTD2(143279); CPEB3(22849); DCLRE1A(9937); GPIAP1(4076); |
| | | LOH11CR2A(4013); SESN3(143686); CCND2(894); APOLD1(81575); |
| | | KNTC1(9735); BCAT1(586); CDK4(1019); PAWR(5074); KITLG(4254); BTG1 |
| | | (694); CHFR(55743); CDK8(1024); NEK3(4752); LIG4(3981); BCL2L2 |
| | | (599); PSEN1(5663); VASH1(22846); CALM1(801); DAD1(1603); CIDEB |
| | | (27141); ACTN1(87); CHES1(1112); BAG5(9529); FGF7(2252); AQP9(366); |
| | | RNF111(54778); BMF(90427); PYGO1(26108); FLJ22795(80154); TRAF7 |
| | | (84231); DNASE1(1773); GSPT1(2935); XYLT1(64131); TNFSF12(8742); |
| | | FZD2(2535); IGF2BP1(10642); ABCC3(8714); TLK2(11011); ZNF624(57547); |
| | | BRCA1(672); MPO(4353); EXOC7(23265); AES(166); MATK(4145); |
| | | NCOA3(8202); UBE2V1(7335); SYCP2(10388); CABLES2(81928); SON(6651); |
| | | GAS2L1(10634); EP300(2033); MAPK8IP2(23542); STAG2(10735); |
| | | DKC1(1736); SYBL1(6845); KLHL15(80311); |
| hsa-miR-624 | 2098; | TARDBP(23435); TNFRSF1B(7133); NBL1(4681); RBBP4(5928); NFYC |
| | | (4802); C1orf84(149469); SH3GLB1(51100); SLC30A7(148867); BCL9(607); |
| | | UHMK1(127933); SMYD2(56950); TNFRSF9(3604); FNDC5(252995); FAF1 |
| | | (11124); SASS6(163786); NOTCH2(4833); PYGO2(90780); PTGS2(5743); |
| | | GLRX2(51022); NUCKS1(64710); LPGAT1(9926); TP53BP2(7159); WDR26 |
| | | (80232); RHOB(388); CASP8(841); CD28(940); PREB(10113); EIF2AK2 |
| | | (5610); TIA1(7072); MCM6(4175); ARL5A(26225); CD302(9936); ALS2CR15 |
| | | (130026); VHL(7428); CTNNB1(1499); GNL3(26354); APPL(26060); |
| | | MITF(4286); MCM2(4171); FAIM(55179); FXR1(8087); TP73L(8626); OPA1 |
| | | (4976); RAF1(5894); RPL32(6161); AXUD1(64651); FOXP1(27086); PTPLB |
| | | (201562); PIK3CB(5291); TBL1XR1(79718); WHSC1(7468); CD38 |
| | | (952); ANAPC4(29945); TRIM2(23321); APBB2(323); CLOCK(9575); DKK2 |
| | | (27123); NEK1(4750); CASP3(836); ERBB2IP(55914); RAD17(5884); APC |
| | | (324); CSNK1G3(1456); GRPEL2(134266); PPP2CA(5515); RIPK1(8737); |
| | | AOF1(221656); TNFAIP3(7128); AKAP12(9590); MDC1(9656); DAXX(1616); |
| | | DST(667); BCLAF1(9774); MAP3K5(4217); LATS1(9113); LMTK2(22853); |
| | | RINT1(60561); CUL1(8454); PMS2(5395); SOSTDC1(25928); IGF2BP3 |
| | | (10643); TMED4(222068); MAGI2(9863); CDK6(1021); EFHA2(286097); |
| | | UNC5D(137970); PAG1(55824); UNC13B(10497); VPS13A(23230); INVS |
| | | (27130); TAF1L(138474); KIAA0367(23273); TSC1(7248); LOC401565 |
| | | (401565); SIRT1(23411); SGPL1(8879); KIF11(3832); LZTS2(84445); BTRC |
| | | (8945); CSPG6(9126); PARD3(56288); DCLRE1A(9937); MKI67(4288); |
| | | hfl-B5(10480); API5(8539); FLJ22104(65084); BIRC3(330); BIRC2(329); |
| | | CUL5(8065); SNF1LK2(23235); FLJ710726(55216); CHEK1(1111); EIF4G2 |
| | | (1982); COPB(1315); RELA(5970); MTL5(9633); SHANK2(22941); ZF |
| | | (58487); SESN3(143686); CASP4(837); CASP5(838); PPP2R1B(5519); ZW10 |
| | | (9183); IAPP(3375); ESPL1(9700); PDE1B(5153); DKFZP586D0919(25895); |
| | | MDM2(4193); SOCS2(8835); RAN(5901); LRP6(4040); YAF2(10138); |
| | | ADCY6(112); MCRS1(10445); FAIM2(23017); FGF9(2254); CDK8(1024); |
| | | BRCA2(675); RFP2(10206); ING1(3621); NUBPL(80224); CGRRF1(10668); |
| | | C14orf35(122830); MNAT1(4331); NRXN3(9369); EGLN3(112399); NFKBIA |
| | | (4792); C14orf39(317761); MOAP1(64112); AP4E1(23431); SLC12A6 |
| | | (9990); MYEF2(50804); TRPM7(54822); MKL2(57496); GSPT1(2935); BOLA2 |
| | | (552900); FTS(64400); CMTM4(146223); APPBP1(8883); SLC12A4 |
| | | (6560); SMPD3(55512); MPHOSPH6(10200); BIRC4BP(54739); CCL2(6347); |
| | | NGFR(4804); MYST2(11143); MSI2(124540); TLK2(11011); PRKCA(5578); |
| | | GAS7(8522); C17orf42(79736); LOC162427(162427); BRCA1(672); |
| | | CDC27(996); ERN1(2081); RNMT(8731); CABLES1(91768); DCC(1630); |
| | | ROCK1(6093); ESCO1(114799); SMAD2(4087); TXNL1(9352); BCL2(596); |
| | | UHRF1(29128); CASP14(23581); PIK3R2(5296); ZNF431(170959); AXL |
| | | (558); ZNF480(147657); ZNF264(9422); ZNF547(284306); AES(166); ZNF100 |
| | | (163227); MCM8(84515); CEP250(11190); CTNNBL1(56259); STK4(6789); |
| | | RAB22A(57403); SLC23A2(9962); SYCP2(10388); USP16(10600); SON |
| | | (6651); BTG3(10950); APP(351); TNRC6B(23112); MAPK1(5594); LIF |
| | | (3976); RBM9(23543); CCNB3(85417); TAF1(6872); NXT2(55916); AKAP14 |
| | | (158798); RP6-213H19.1(51765); CXorf38(159013); SMC1L1(8243); PHKA1 |
| | | (5255); LAMP2(3920); ARHGEF6(9459); MTCP1(4515); CYorf15B |
| | | (84663); |
| hsa-miR-625 | 2099; | TNFRSF1B(7133); PAX7(5081); LDLRAD2(401944); CDC42(998); ARID1A |
| | | (8289); RCC1(1104); RBBP4(5928); NFYC(4802); CDC20(991); PLK3(1 |

Table 4-132

263); PRPF38B(55119); CSF1(1435); PROK1(84432); PTGFRN(5738); FAM46C
(54855); RBM8A(9939); BCL9(607); S100A7L1(338324); C1orf60(65123);
UBAP2L(9898); CKS1B(1163); IGSF4B(57863); PEA15(8682); MPZL1
(9019); CACNA1E(777); PPP1R12B(4660); PRELP(5549); ATP2B4(493);
SNRPE(6635); CCNL2(81669); TNFRSF14(8764); TNFRSF9(3604);
MTHFR(4524); FBXO42(54455); E2F2(1870); EYA3(2140); BSDC1(55108);
YARS(8565); CSMD2(114784); CSF3R(1441); ELOVL1(64834); TESK2
(10420); PIK3R3(8503); AGBL4(84871); TACSTD2(4070); SSX2IP(117178);
GFI1(2672); C1orf183(55924); NRAS(4893); TRIM45(80263); ARNT(405);
GATAD2B(57459); ADAR(103); PYGO2(90780); COPA(1314); DEDD
(9191); PHLDA3(23612); SYT2(127833); PLEKHA6(22874); LPGAT1(9926);
SUSD4(55061); WDR26(80232); ENAH(55740); C1orf198(84886); SELI
(85465); ANTXR1(84168); MXD1(4084); SMYD5(10322); HTRA2(27429);
BRRN1(23397); BCL2L11(10018); ACTR3(10096); RIF1(55183); ZAK(51776);
ABI2(10152); COL4A3(1285); TMEM16G(50636); SEPT2(4735); HS1BP3
(64342); FLJ21945(80304); CGREF1(10669); SERTAD2(9792); SPRED2
(200734); MOBK1B(55233); C2orf3(6936); EDAR(10913); STK17B(9262);
C2orf19(394261); OGG1(4968); NR2C2(7182); ZNF501(115560); LIMD1
(8994); TDGF1(6997); TREX1(11277); ARIH2(10425); IL17RB(55540);
APPL(26060); ZPLD1(131368); ACPP(55); CLDN11(5010); ABCF3(55324);
PAK2(5062); NKIRAS1(28512); AXUD1(64651); ZNF445(353274); FHIT
(2272); EIF4E3(317649); FLJ46210(389152); SIAH2(6478); KPNA4
(3840); IDUA(3425); WHSC1(7468); HD(3064); MAN2B2(23324); CD38(952);
HCAP-G(64151); HIP2(3093); RASL11B(65997); SEPT11(55752); CCNG2
(901); NFKB1(4790); MAB21L2(10586); LOC285498(285498); DKFZp761B107
(91050); G3BP2(9908); CCNI(10983); SFRP2(6423); CASP3(836);
NIPBL(25836); ARSK(153642); CSNK1GS(1456); RAD50(10111); PCDHGA1
(56114); KIAA0141(9812); SLC36A1(206358); SGCD(6444); ZNF346
(23567); NSD1(64324); DAP(1611); PRLR(5618); PRKAA1(5562); P18SRP
(285672); ZBED3(84327); MCC(4168); SEMA6A(57556); IRF1(3659); AFF4
(27125); SPOCK1(6695); CDC25C(995); DIAPH1(1729); SPRY4(81848);
FGF1(2246); LARS(51520); E2F3(1871); C6orf134(79969); AIF1(199); HMGA1
(3159); SCUBE3(222663); BRPF3(27154); KIAA0082(23070); GLP1R
(2740); PARC(23113); TMEM63B(55362); IL17(3665); TTK(7272); ANKRD6
(22881); TBPL1(9519); HECA(51696); ESR1(2099); QKI(9444); ELOVL2
(54898); NEDD9(4739); ATXN1(6310); GABBR1(2550); IER3(8870);
DAXX(1616); BAK1(578); IHPK3(117283); CCND3(896); RDS(5961); DST
(667); ATG5(9474); SNX3(8724); BCLAF1(9774); PLAGL1(5325); LATS1
(9113); RPS6KA2(6196); TTYH3(80727); HDAC9(9734); ZNRF2(223082);
SEPT7(989); KIAA1706(80820); EGFR(1956); STEAP2(261729); ZNF498
(221785); STAG3(10734); BCAP29(55973); TRIAD3(54476); HOXA3(3200);
CARD4(10392); INHBA(3624); TMED4(222068); PURB(5814); CALN1
(83698); GATS(352954); LRCH4(4034); RASA4(10156); FLJ44186(346689);
HIPK2(28996); BRAF(673); TNKS(8658); BNIP3L(665); RBPMS(11030);
UNC5D(137970); TACC1(6867); UBE2V2(7336); SULF1(23213); E2F5
(1875); LAPTM4B(55353); WISP1(8840); PINX1(54984); CTSB(1508); CNOT7
(29883); LZTS1(11178); BIN3(55909); TNFRSF10B(8795); STC1(6781);
ADRA1A(148); ZNF395(55893); DUSP4(1846); SFRP1(6422); PAG1(55824);
TP53INP1(94241); RAD21(5885); EXT1(2131); DERL1(79139); MTSS1(9788);
CYP11B1(1584); UBE2R2(54926); CBWD3(445571); SPIN(10927);
C9orf47(286223); INVS(27130); PAPPA(5069); CDK9(1025); PKN3(29941);
PSIP1(11168); IFNW1(3467); CDKN2A(1029); GAS1(2619); SEMA4D
(10507); EPB41L4B(54566); ROD1(9991); HDHD3(81932); AKNA(80709);
FBXW2(26190); TRAF1(7185); PPP6C(5537); TSC1(7248); C9orf140
(89958); NELF(26012); MRC1L1(414308); ZNF365(22891); SGPL1(8879);
FAS(355); CNNM1(26507); LZTS2(84445); SUFU(51684); PDCD11(22984);
TCF7L2(6934); C10orf81(79949); KIAA1600(57700); BAG3(9531); GPR26
(2849); LARP5(23185); CXCL12(6387); MARCH8(220972); PPP3CB
(5532); SFRP5(6425); SEC31L2(25956); FBXW4(6468); SH3PXD2A(9644);
KIAA1754(85450); SMNDC1(10285); C10orf46(143384); BNIP3(664); STIM1
(6786); WEE1(7465); MLSTD2(84188); HIPK3(10114); TSPAN18(90139);
MDK(4192); DPF2(5977); SSSCA1(10534); DIPA(11007); PELI3(246330);
FBXL11(22992); CCND1(595); FLJ10661(55199); PLEKHB1(58473);
CAPN5(726); BIRC2(329); DUSP8(1850); IGF2(3481); APBB1(322); SOX6
(55553); AGTRL1(187); GPR44(11251); BAD(572); MAP3K11(4296); RE

Table 4-133

| | | |
|---|---|---|
| | | LA(5970); AYP1(84153); MTL5(9633); FGF3(2248); PAK1(5058); LOC283219 |
| | | (283219); SESN3(143686); PPP2R1B(5519); EVA1(10205); BCL9L(283149); |
| | | HYOU1(10525); CCND2(894); PTPN6(5777); PEX5(5830); LOC387856 |
| | | (387856); AQP2(359); ESPL1(9700); SP1(6667); CDK2(1017); RAB5B |
| | | (5869); MDM2(4193); NUDT4(11163); KIAA0789(9671); TPCN1(53373); |
| | | BCL7A(605); SETD8(387893); RAN(5901); VAMP1(6843); ING4(51147); |
| | | GRIN2B(2904); GYS2(2998); LALBA(3906); FAIM2(23017); LOC401720 |
| | | (401720); ATF7(11016); CALCOCO1(57658); SMUG1(23583); CBX5(23468); |
| | | ZNF385(25946); CDK4(1019); CTDSP2(10106); UBE2N(7334); PCTK2 |
| | | (5128); NT5DC3(51559); FAM109A(144717); RHOF(54509); MGC9850(219404); |
| | | AKAP11(11215); RFP2(10206); FLJ40296(122183); ARHGEF7(8874); |
| | | DCAMKL1(9201); FOXO1A(2308); LCP1(3936); LOC283487(283487); |
| | | PNN(5411); RGS6(9628); C14orf118(55668); VASH1(22846); CALM1(801); |
| | | AMN(81693); ACIN1(22985); STRN3(29966); FOXA1(3169); RAB15 |
| | | (376267); ZFP36L1(677); C14orf43(91748); PGF(5228); TGFB3(7043); ANGEL1 |
| | | (23357); C14orf103(55102); PPP1R13B(23368); AKT1(207); SORD |
| | | (6652); Gcom1(145781); SMAD3(4088); THSD4(79875); ARIH1(25820); CCDC33 |
| | | (80125); CSK(1445); SCAMP5(192683); LOC440295(440295); ST8SIA2 |
| | | (8128); IGF1R(3480); BMF(90427); MYEF2(50804); BCL2L10(10017); |
| | | PYGO1(26108); CSNK1G1(53944); PDCD7(10081); PKM2(5315); CHRNB4 |
| | | (1143); NTRK3(4916); C15orf38(348110); LYSMD4(145748); C16orf34 |
| | | (90861); TBL3(10607); TSC2(7249); KREMEN2(79412); A2BP1(54715); |
| | | ATXN2L(11273); ATP2A1(487); SPN(6693); FBXL19(54620); PAPD5(64282); |
| | | CYLD(1540); HSPC065(29070); CYB5B(80777); WWP2(11060); PDPR |
| | | (55066); KIAA0513(9764); FLJ20186(54849); PKMYT1(9088); N-PAC(84656); |
| | | GSPT1(2935); SMG1(23049); ERN2(10595); STX1B2(112755); SLAH1 |
| | | (6477); CMTM4(146223); SLC12A4(6560); MINK1(50488); NLGN2(57555); |
| | | MYOCD(93649); WSB1(26118); NLK(51701); TRAF4(9618); NF1(4763); |
| | | LIG3(3980); AP2B1(163); MLLT6(4302); LASP1(3927); THRA(7067); |
| | | CNTNAP1(8506); FZD2(2535); ADAM11(4185); MAPT(4137); UBE2Z(65264); |
| | | IGF2BP1(10642); NGFR(4804); NXPH3(11248); CROP(51747); STXBP4 |
| | | (252983); YPEL2(388403); RPS6KB1(6198); TLK2(11011); KPNA2(3838); |
| | | BAIAP2(10458); FN3K(64122); UNQ5783(388325); GPS2(2874); VAMP2 |
| | | (6844); GAS7(8522); C17orf63(55731); FLJ44815(400591); CCL3L3 |
| | | (414062); CACNB1(782); NEUROD2(4761); ORMDL3(94103); HDAC5(10014); |
| | | HOXB3(3213); HOXB5(3215); DLX3(1747); COL1A1(1277); SFRS1 |
| | | (6426); ERN1(2081); SFRS2(6427); CBX4(8535); TBC1D16(125058); VAPA |
| | | (9218); CEP192(55125); CABLES1(91768); CCDC5(115106); C18orf25(147339); |
| | | KATNAL2(83473); MAPK4(5596); PHLPP(23239); C18orf4(92126); |
| | | CNN2(1265); MIDN(90007); MUM1(84939); CHAF1A(10036); PDE4A(5141); |
| | | DNM2(1785); NFIX(4784); ZSWIM4(65249); MAP3K10(4294); SNRPA |
| | | (6626); AXL(558); BCL3(602); LOC400707(400707); SPHK2(56848); LOC284361 |
| | | (284361); PPP2R1A(5518); CACNG7(59284); ZNF444(55311); |
| | | MGC2752(65996); SLC39A3(29985); AES(166); MATK(4145); ZBTB7A(51341); |
| | | PTPRS(5802); PRKACA(5566); JUND(3727); ZNF585A(199704); SIRT2 |
| | | (22933); FLJ36888(126526); TGFB1(7040); DEDD2(162989); BBC3 |
| | | (27113); SLC8A2(6543); KLK10(5655); SNPH(9751); CDC25B(994); PLCB1 |
| | | (23236); PAX1(5075); HM13(81502); EPB41L1(2036); C20orf121(79183); |
| | | KCNK15(60598); STK4(6789); ZGPAT(84619); SCRT2(85508); FKBP1A |
| | | (2280); OVOL2(58495); BCL2L1(598); CDH22(64405); ELMO2(63916); PTGIS |
| | | (5740); UBE2V1(7335); CABLES2(81928); DIDO1(11083); ITSN1(6453); |
| | | TTC3(7267); RUNX1(861); SEPT5(5413); RAB36(9609); SMARCB1(6598); |
| | | FLJ33814(150275); KREMEN1(83999); NF2(4771); TNRC6B(23112); |
| | | EP300(2033); ZC3H7B(23264); MN1(4330); RBM9(23543); CARD10(29775); |
| | | DDX17(10521); PDGFB(5155); NHP2L1(4809); PKDREJ(10343); |
| | | RP13-297E16.1(8227); TBL1X(6907); YIPF6(286451); TMEM28(27112); |
| | | EDA(1896); MLLT7(4303); IL13RA1(3597); BIRC4(331); BCORL1(63035); |
| | | PNMA6A(84968); DUSP9(1852); PLXNB3(5365); TAZ(6901); GDI1(2664); |
| | | PPP2R3B(28227); TFE3(7030); SMC1L1(8243); PCDH19(57526); BTK |
| | | (695); SEPT6(23157); CUL4B(8450); APLN(8862); CETN2(1069); BCAP31 |
| | | (10134); HCFC1(3054); MECP2(4204); |
| hsa-miR-627 | 2100; | LDLRAD2(401944); KHDRBS1(10657); RBBP4(5928); MACF1(23499); C1orf83 |
| | | (127428); CDC7(8317); SLC30A7(148867); PROK1(84432); SYCP1 |
| | | (6847); PEA15(8682); C1orf27(54953); TRAF5(7188); CIAS1(114548); CDC2L2 |
| | | (985); RCC2(55920); CLSPN(63967); KIAA0494(9813); FAF1(11124 |

Table 4-134

| | | |
|---|---|---|
| | | ); SLC35D1(23169); PTGER3(5733); TGFBR3(7049); NOTCH2(4853); RORC |
| | | (6097); CD84(8832); ASPM(259266); SYT2(127833); TP53BP2(7159); |
| | | SOX11(6664); BIRC6(57448); PRKCE(5581); HK2(3099); MGC52057(130574); |
| | | RIF1(55183); GPD2(2820); CFLAR(8837); COL4A3(1285); CAPN10 |
| | | (11132); SEPT2(4735); YWHAQ(10971); CARD12(58484); HAAO(23498); |
| | | CREG2(200407); EDAR(10913); BUB1(699); IFIH1(64135); STK17B(9262); |
| | | BARD1(580); PAX3(5077); C2orf19(394261); HDAC4(9759); FANCD2 |
| | | (2177); VHL(7428); NR2C2(7182); ZNF502(91392); LIMD1(8994); HEMK1 |
| | | (51409); ABHD6(57406); ZNF654(55279); PLXNA1(5361); SKIL(6498); FAM79B |
| | | (285386); KIAA1143(57456); AMT(275); PIK3CB(5291); ATR(545); |
| | | HD(3064); MAN2B2(23324); SEPT11(55752); CCNG2(901); FGF5(2250); |
| | | FLJ13273(79807); FGF2(2247); PLK4(10733); SFRP2(6423); VEGFC(7424); |
| | | ERBB2IP(55914); RAD17(5884); RGMB(285704); APC(324); RAD50 |
| | | (10111); SGCD(6444); SEMA5A(9037); PRLR(5618); PRKAA1(5562); ARL15 |
| | | (54622); P18SRP(285672); MCC(4163); SRA1(10011); DOK3(79930); LY86 |
| | | (9450); GMNN(51053); HFE(3077); ZNF192(7745); AIF1(199); SCUBE3 |
| | | (222663); IL17(3605); KIAA1900(114792); LOC619208(619208); TBPL1 |
| | | (9519); HECA(51696); SASH1(23328); WTAP(9589); RANBP9(10048); SUPT3H |
| | | (8464); MCM3(4172); DST(667); BACH2(60468); PDCD2(5134); SEPT7 |
| | | (989); EGFR(1956); STAG3(10734); IRF5(3663); MKLN1(4289); TRIAD3 |
| | | (54476); SOSTDC1(25928); CARD4(10392); CALN1(83698); YWHAG |
| | | (7532); CDK6(1021); CDK5(1020); TNFRSF10C(8794); ESCO2(157570); |
| | | UNC5D(137970); BAG4(9530); TACC1(6867); SULF1(23213); RIPK2(8767); |
| | | EBAG9(9166); WISP1(8840); PINX1(54984); SFRP1(6422); PAG1(55824); |
| | | FBXO32(114907); MGC21881(389741); NTRK2(4915); C9orf47(286223); |
| | | IFNA2(3440); CDKN2B(1030); BAG1(573); NOL6(65083); IGFBPL1 |
| | | (347252); TNFSF8(944); CIZ1(25792); FAM78A(286336); TSC1(7248); ARL5B |
| | | (221079); C10orf9(219771); SIRT1(23411); EIF4EBP2(1979); FAS(355); |
| | | SUFU(51684); DLG5(9231); VAX1(11023); MKI67(4288); WEE1(7465); |
| | | TMEM86A(144110); HIPK3(10114); VEGFB(7423); SLCO2B1(11309); AMOTL1 |
| | | (154810); ATM(472); SNF1LK2(23235); TAGLN(6876); CD3G(917); |
| | | SLC1A2(6506); HRASLS3(11145); SUV420H1(51111); FZD4(8322); CASP1 |
| | | (834); FLJ45803(399948); PPP2R1B(5519); ZW10(9183); SCN3B(55800); |
| | | TSPAN9(10867); NTF3(4908); PTPN6(5777); BCL2L14(79370); TMEM16F |
| | | (196527); FLJ20489(55652); SMARCD1(6602); ESPL1(9700); CDK2 |
| | | (1017); APAF1(317); KIAA1853(84530); P2RX7(5027); KNTC1(9735); NOL1 |
| | | (4839); VDR(7421); CBX5(23468); PAWR(5074); TMCC3(57458); DIP13B |
| | | (55198); FAM109A(144717); MPHOSPH9(10198); CUL4A(8451); LIG4 |
| | | (3981); PSEN1(5663); KCNK13(56659); BDKRB2(624); ACIN1(22985); |
| | | TRIM9(114088); SYNJ2BP(55333); LTBP2(4053); NEK9(91754); PPP1R13B |
| | | (23368); BUB1B(701); TMEM62(80021); KIF23(9493); BMF(90427); |
| | | BNIP2(663); C15orf38(348110); E4F1(1877); CARD15(64127); CYLD(1540); |
| | | SLC7A6(9057); ERN2(10595); SMPD3(55512); HIC1(3090); MAP2K4 |
| | | (6416); NF1(4763); WIRE(147179); NGFR(4804); CROP(51747); PPM1D(8493); |
| | | TLK2(11011); KPNA2(3838); C17orf27(57674); GPS1(2873); P2RX1 |
| | | (5023); NALP1(22861); TIAF1(9220); PLXDC1(57125); ZNF652(22834); SEPT4 |
| | | (5414); CBX4(8535); C18orf1(753); MALT1(10892); SERPINB2(5055); |
| | | ROCK1(6093); ESCO1(114799); FSTL3(10272); TNFSF9(8744); PIK3R2 |
| | | (5296); AXL(558); FOSB(2354); SPHK2(56848); LOC147650(147650); |
| | | LILRB4(11006); NALP2(55655); ZNF264(9422); LOC390980(390980); HKR2 |
| | | (342945); MATK(4145); TNFSF14(8740); ZNF443(10224); DPF1(8193); |
| | | CEACAM7(1087); KCNA7(3743); ATRN(8455); KIF3B(9371); CEP250 |
| | | (11190); BIRC7(79444); BCL2L1(598); C20orf112(140688); RBM12(10137); |
| | | SLC35C2(51006); DIDO1(11083); SON(6651); CHAF1B(8208); MX1(4599); |
| | | BRWD1(54014); ADORA2A(135); KREMEN1(83999); KDELR3(11015); |
| | | EP300(2033); CTA-216E10.6(79640); SEPT3(55964); MAPK8IP2(23542); |
| | | C22orf13(83606); SMC1L2(27127); RP11-114H20.1(401589); SRPX2 |
| | | (27286); ARMCX4(158947); BIRC4(331); STAG2(10735); SPANX-N1(494118); |
| | | SPANX-N5(494197); BTK(695); PDCD8(9131); ARHGEF6(9459); HCFC1 |
| | | (3054); |
| hsa-miR-630 | 2101; | TNFRSF1B(7133); PRAMEF9(343070); CLIC4(25932); RBBP4(5928); UTP11L |
| | | (51118); MACF1(23499); NSUN4(387338); LRRC8C(84230); CDC7 |
| | | (8317); CDC14A(8556); SYCP1(6847); BCL9(607); DDR2(4921); PPP1R12B |
| | | (4660); PROX1(5629); FRAP1(2475); RCC2(55920); FUSIP1(10772); SASS6 |
| | | (163786); ST7L(54879); NOTCH2(4853); MEF2D(4209); ASPM(2592 |

Table 4-135

| | | |
|---|---|---|
| | | 66); ZBTB41(360023); USH2A(7399); ESRRG(2104); ERO1LB(56605); FLJ40869 |
| | | (348654); RHOB(388); BIRC6(57448); MSH2(4436); BRRN1(23397); |
| | | HNMT(3176); MGC52057(130574); RIF1(55183); ZAK(51776); CD28(940); |
| | | COL4A3(1285); YWHAQ(10971); CGREF1(10669); KCNG3(170850); |
| | | PSME4(23198); RTN4(57142); BUB1(699); ARL5A(26225); TLK1(9874); |
| | | KIAA1715(80856); NCKAP1(10787); STK17B(9262); PAX3(5077); SKIP |
| | | (80309); FANCD2(2177); PCAF(8850); CTNNB1(1499); TDGF1(6997); ARMC8 |
| | | (25852); ACPL2(92370); SMC4L1(10051); PRKCI(5584); TP73L(8626); |
| | | CRBN(51185); EIF4E3(317649); CGGBP1(8545); PIK3CB(5291); FGF12 |
| | | (2257); MGC21675(92070); HCAP-G(64151); TMPRSS11E(28983); ARHGAP24 |
| | | (83478); TMEM34(55751); TLR2(7097); TDO2(6999); GRIA2(2891); |
| | | WWC2(80014); DUX4(22947); CCDC4(389206); GABRA4(2557); CCNI |
| | | (10983); PRKG2(5593); SCD5(79966); WDFY3(23001); NEK1(4750); FAM105B |
| | | (90268); SUB1(10923); NIPBL(25836); CARD6(84674); ERBB2IP |
| | | (55914); SLC30A5(64924); APC(324); COMMD10(51397); LOC90624(90624); |
| | | BNIP1(662); PRLR(5618); GDNF(2668); FEM1C(56929); TMED7 |
| | | (51014); ATG12(9140); CDC23(8697); RIPK1(8737); PRPF4B(8899); LY86 |
| | | (9450); E2F3(1871); ALDH5A1(7915); ENPP4(22875); PTP4A1(7803); CD109 |
| | | (135228); SART2(29940); NCOA7(135112); STXBP5(134957); QKI(9444); |
| | | ATXN1(6310); MDC1(9656); ENPP5(59084); PKHD1(5314); DST(667); |
| | | DJ122O8.2(57226); BCLAF1(9774); MAP3K5(4217); LATS1(9113); FOXK1 |
| | | (221937); NFE2L3(9603); EGFR(1956); PHTF2(57157); ZNF655(79027); |
| | | HBP1(26959); ING3(54556); TRIM24(8805); INHBA(3624); FLJ36031 |
| | | (168455); BRAF(673); LMBR1(64327); ZNF705CP(389631); SLC39A14(23516); |
| | | SULF1(23213); TERF1(7013); PPM2C(54704); MAL2(114569); SFRP1 |
| | | (6422); CCNE2(9134); STK3(6788); SAMD12(401474); JAK2(3717); RECK |
| | | (8434); MAK10(60560); TRAF2(7186); ZDHHC21(340481); TRIM14(9830); |
| | | ZMYND11(10771); CDC2(983); FAS(355); BTRC(8945); ZRANB1(54764); |
| | | DOCK1(1793); CUL2(8453); SMNDC1(10285); GPAM(57678); DCLRE1A |
| | | (9937); HIPK3(10114); YAP1(10413); BIRC2(329); ATM(472); PSMA1 |
| | | (5682); WT1(7490); RAB30(27314); SESN3(143686); ARHGAP20(57569); |
| | | ASAM(79827); LTBR(4055); SOCS2(8835); SLC25A3(5250); APAF1(317); |
| | | KNTC1(9735); FLJ46363(400002); LRP6(4040); LMO3(55885); SLC2A13 |
| | | (114134); SLC38A1(81539); CBX5(23468); DDIT3(1649); WIF1(11197); |
| | | PAWR(5074); DUSP6(1848); PCTK2(5128); ZNF605(90462); FGF9(2254); |
| | | HS6ST3(266722); FLJ25477(219287); FOXO1A(2308); BCL2L2(599); TXNDC |
| | | (81542); C14orf108(55745); C14orf35(122830); MNAT1(4331); SLC39A9 |
| | | (55334); YY1(7528); DAD1(1603); CFL2(1073); DLG7(9787); C14orf103 |
| | | (55102); BUB1B(701); IGF1R(3480); AVEN(57099); FLJ20582(54989); |
| | | MYO5A(4644); ARPP-19(10776); MKL2(57496); CHD9(80205); CTCF(10664); |
| | | WWOX(51741); ERN2(10595); PHLPPL(23035); SLC2A4(6517); MAPK7 |
| | | (5598); FLJ46247(374786); RNF135(84282); NF1(4763); RHOT1(55288); |
| | | TADA2L(6871); IGF2BP1(10642); CROP(51747); TLK2(11011); NCOR1 |
| | | (9611); BECN1(8678); BRCA1(672); MPO(4353); SEPT4(5414); CABLES1 |
| | | (91768); PHLPP(23239); DOK6(220164); KIAA0863(22850); ESCO1 |
| | | (114799); PPP2R1A(5518); ZNF562(54811); ZFP260(339324); ZNF585A(199704); |
| | | CEACAM1(634); CARD8(22900); MCM8(84515); PLCB1(23236); |
| | | ARFGEF2(10564); PARD6B(84612); CSNK2A1(1457); RBL1(5933); CABLES2 |
| | | (81928); USP16(10600); C21orf91(54149); EP300(2033); CTA-250D10.11 |
| | | (164684); MAPK1(5594); RBM9(23543); TOB2(10766); MSL3L1(10943); |
| | | HDAC6(10013); YIPF6(286451); ARMCX4(158947); BIRC4(331); IKBKG |
| | | (8517); ZDHHC15(158866); MTCP1(4515); CLIC2(1193); |
| hsa-miR-631 | 2102; | SKI(6497); KCNAB2(8514); RP13-15M17.2(199953); TNFRSF1B(7133); |
| | | EPHA8(2046); TXLNA(200081); HDAC1(3065); RBBP4(5928); MACF1(23499); |
| | | PROK1(84432); BCL9(607); ANXA9(8416); ZBTB7B(51043); C1orf106 |
| | | (55765); TGFB2(7042); TNFRSF14(8764); CHD5(26038); CTNNBIP1 |
| | | (56998); DFFA(1676); FRAP1(2475); MAD2L2(10459); C1orf126(200197); |
| | | CASP9(842); PSMB2(5690); TESK2(10420); GFI1(2672); PAP2D(163404); |
| | | CRP(1401); KCNJ10(3766); LAD1(3898); LMOD1(25802); FAIM3(9214); |
| | | CD34(947); LYST(1130); FLJ30851(375190); PPP1CB(5500); HTRA2(27429); |
| | | HK2(3099); DLX1(1745); DNAJB2(3300); COL4A3(1285); DGKD(8527); |
| | | CGREF1(10669); FLJ34931(388939); MCFD2(90411); TGFA(7039); EDAR |
| | | (10913); TLK1(9874); STK17B(9262); FZD5(7855); TNS1(7145); CUL3 |
| | | (8452); STK25(10494); VHL(7428); HDAC11(79885); NR2C2(7182); ZNF662 |
| | | (389114); RBM5(10181); PPM1M(132160); FAM62C(83850); IMPD |

Table 4-136

H2(3615); TRAIP(10293); BAP1(8314); NEK4(6787); TOMM70A(9868); MGLL
(11343); STAG1(10274); ATR(545); ALG3(10195); HD(3064); FLJ35725
(152992); EREG(2069); FGF2(2247); GAK(2580); PPP2R2C(5522); ANAPC10
(10393); SPATA4(132851); GZMA(3001); APC(324); RAD50(10111);
TCF7(6932); PHF15(23338); PSD2(84249); PCDHB9(56127); PCDHGB4
(8641); PCDHGA1(56114); N4BP3(23138); DAP(1611); PRKAA1(5562); UNG2
(10309); MARCH3(115123); PPP2CA(5515); NRG2(9542); FBXW11
(23291); E2F3(1871); TNF(7124); CSNK2B(1460); TTBK1(84630); VEGF
(7422); RWDD2(112611); PEX3(8504); NEDD9(4739); MDC1(9656); TAPBP
(6892); BAK1(578); IHPK3(117283); CUL7(9820); ATG5(9474); FRK(2444);
PLAGL1(5325); LATS1(9113); PDCD2(5134); CYP2W1(54905); SEPT7
(989); DKFZp761I2123(83637); ADCY1(107); EGFR(1956); FZD9(8326); STAG3
(10734); NYREN18(51667); LOC90639(90639); TRIAD3(54476); PMS2
(5395); INHBA(3624); LOC401357(401357); PODXL(5420); AKR1B1
(231); HIPK2(28996); POLR3D(661); TNFRSF10C(8794); BNIP3L(665);
C8orf44(56260); MYC(4609); WISP1(8840); RHPN1(114822); PINX1(54984);
FAM86B1(85002); BIN3(55909); TNFRSF10B(8795); TNFRSF10D(8793);
TRIM35(23087); CLU(1191); DUSP4(1846); SFRP1(6422); ANK1(286);
STK3(6788); EXT1(2131); TNFRSF11B(4982); SCRT1(83482); ANKRD15
(23189); RECK(8434); NTRK2(4915); DAPK1(1612); FGD3(89846); OLFML2A
(169611); TBC1D13(54662); ABL1(25); CDKN2B(1030); TAF1L
(138474); TRIM14(9830); TNFSF15(9966); LHX6(26468); ASB6(140459);
FAM78A(286336); UCK1(83549); ANAPC2(29882); GTPBP4(23560); WDR37
(22884); ANXA8(244); ZNF365(22891); SFTPA1(6435); PTEN(5728);
KIF11(3832); FRAT1(10023); PI4KII(55361); BTRC(8945); NOLC1(9221);
SUFU(51684); CASC2(255082); BAG3(9531); GPR26(2849); ZRANB1(54764);
DOCK1(1793); PTPRE(5791); ADARB2(105); NEBL(10529); FZD8
(8325); LRRC20(55222); DLG5(9231); SFTPA2(6436); VAX1(11023); FAM53B
(9679); MKI67(4288); PARVA(55742); EXT2(2132); TSPAN18(90139);
LRFN4(78999); FLJ10661(55199); CEP57(9702); BIRC2(329); CUL5(8065);
TAGLN(6876); CD3E(916); EI24(9538); POLR2L(5441); IGF2(3481); TPP1
(1200); ACP2(53); VPS37C(55048); HRASLS3(11145); TRPT1(83707);
CDCA5(113130); FOSL1(8061); BRMS1(25855); PPP1CA(5499); MTL5
(9633); SHANK2(22941); PHOX2A(401); PAK1(5058); FZD4(8322); SLC35F2
(54733); PPP2R1B(5519); BCL9L(283149); CCND2(894); NTF3(4908);
PTMS(5763); BCL2L14(79370); DDX11(1663); FLJ20489(55652); PRPF40B
(25766); ESPL1(9700); TPCN1(53373); RAN(5901); SFRS8(6433); FGF6
(2251); ING4(51147); RBP5(83758); BCAT1(586); LALBA(3906); MCRS1
(10445); FMNL3(91010); FAIM2(23017); ATF7(11016); OASL(8638); ABCB9
(23457); PITPNM2(57605); FGF9(2254); RFP2(10206); ING1(3621); LATS2
(26524); LIG4(3981); BCL2L2(599); VASH1(22846); TRAF3(7187);
KIAA0125(9834); DLG7(9787); SPTB(6710); DPF3(8110); TGFB3(7043);
SPTLC2(9517); AKT1(207); PAK6(56924); TLN2(83660); KIF23(9493); AKAP13
(11214); ABHD2(11057); IGF1R(3480); DUOX2(50506); MYO5A(4644);
CSNK1G1(53944); ACSBG1(23205); FLJ39743(283777); C16orf9(83986);
FLJ36208(283948); KIAA1924(197335); UBE2I(7329); KIAA0683
(9894); TBL3(10607); TSC2(7249); GBL(64223); FLYWCH1(84256); PMM2
(5373); KIAA0350(23274); SULT1A3(6818); PRRT2(112476); SULT1A4
(445329); NOL3(8996); NFAT5(10725); CDK10(8558); AXIN1(8312); C16orf28
(65259); PKMYT1(9088); ERN2(10595); STX1B2(112755); CMTM4(146223);
LOC283849(283849); SLC12A4(6560); MPHOSPH6(10200); GSG2
(83903); FGF11(2256); KIAA0672(9912); HS3ST3B1(9953); MAPK7(5598);
KSR1(8844); RNF135(84282); NMT1(4836); MAPT(4137); NXPH3(11248);
CROP(51747); PRKCA(5578); SLC9A3R1(9368); TMEM104(54868);
CDR2L(30850); KCTD2(23510); BIRC5(332); CARD14(79092); BAIAP2(10458);
MNT(4335); CAMKK1(84254); P2RX1(5023); CLDN7(1366); TP53
(7157); AURKB(9212); GAS2L2(246176); HDAC5(10014); MRPL27(51264);
ERN1(2081); USH1G(124590); RECQL5(9400); TMC6(11322); SOCS3(9021);
TBC1D16(125058); B3GNTL1(146712); GNAL(2774); DCC(1630); PHLPP
(23239); C18orf4(92126); PARD6G(84552); CDC34(997); MIDN(90007);
APC2(10297); FZR1(51343); HMG20B(10362); GIPC3(126326); ALKBH7
(84286); P2RY11(5032); DNM2(1785); RAB8A(4218); LOC115098(115098);
FLJ43870(400686); SAMD4B(55095); AXL(558); ZNF526(116115);
APOE(348); RPS11(6205); NALP2(55655); RPL28(6158); ZNF446(55663);
C19orf19(284451); AES(166); RAKL1(84839); ELAVL3(1995); ZNF490(5

Table 4-137

| | | |
|---|---|---|
| | | 7474); ABHD8(79575); PRX(57716); SHANK1(50944); TRIB3(57761); STK35 |
| | | (140901); CDC25B(994); CDS2(8760); CEP250(11190); TGIF2(60436); |
| | | PPP1R16B(26051); MYBL2(4605); YWHAB(7529); STK4(6789); SLC2A10 |
| | | (81031); NTSR1(4923); ZGPAT(84619); RBL1(5933); ZHX3(23051); LAMA5 |
| | | (3911); DIDO1(11083); GMEB2(26205); SON(6651); ITGB2(3689); DKFZp434K191 |
| | | (29797); TSSK2(23617); SEPT5(5413); ADORA2A(135); MCM5 |
| | | (4174); SYNGR1(9145); EP300(2033); ZBED4(9889); BID(637); DGCR14 |
| | | (8220); LOC128977(128977); FLJ21125(79680); PPM1F(9647); C22orf13 |
| | | (83606); MN1(4330); SSTR3(6753); PDGFB(5155); TOB2(10766); POLR3H |
| | | (171568); PNPLA5(150379); SMC1L2(27127); LOC150383(150383); |
| | | ALG12(79087); FLJ41993(400935); MAPK12(6300); PCTK1(5127); MLLT7 |
| | | (4303); TAF1(6872); ATP1B4(23439); RP6-213H19.1(51765); ATP2B3 |
| | | (492); SPRY3(10251); PPP2R3B(28227); SLC38A5(92745); SMC1L1(8243); |
| | | CUL4B(8450); APLN(8862); FAM11A(84548); FAM58A(92002); HCFC1 |
| | | (3054); |
| hsa-miR-634 | 2103; | PRKCZ(5590); CLCN6(1185); PLA2G2F(64600); ZBTB40(9923); PDIK1L |
| | | (149420); SESN2(83667); EIF2C4(192670); MACF1(23499); GADD45A(1647); |
| | | SH3GLB1(51100); CDC7(8317); FAM102B(284611); CSF1(1435); HIPK1 |
| | | (204851); C1orf60(65123); ATP8B2(57198); CKS1B(1163); UHMK1(127933); |
| | | PBX1(5087); PRRX1(5396); C1orf21(81563); ADORA1(134); ATP2B4 |
| | | (493); SNRPE(6635); CTSE(1510); PFKFB2(5208); TRAF5(7188); KMO |
| | | (8564); UBE2J2(118424); CPSF3L(54973); TNFRSF9(3604); CTNNBIP1 |
| | | (56998); FRAP1(2475); RCC2(55920); TTMB(399474); KIAA0319L(79932); |
| | | CLSPN(63967); YRDC(79693); MUTYH(4595); TESK2(10420); C1orf163 |
| | | (65260); TGFBR3(7049); NRAS(4893); MUC1(4582); DEDD(9191); TEDDM1 |
| | | (127670); KIF21B(23046); LPGAT1(9926); TP53BP2(7159); ENAH |
| | | (55740); PPP1CB(5500); LYCAT(253558); BIRC6(57448); PRKCE(5581); |
| | | PCYOX1(51449); HTRA2(27429); HK2(3099); FLJ20758(55037); ZAK(51776); |
| | | PMS1(5378); CD28(940); ALS2CR19(117583); COL4A3(1285); PSMD1 |
| | | (5707); LOC339768(339768); ASB1(51665); GPC1(2817); BOK(666); CYS1 |
| | | (192668); PPM1G(5496); CARD12(58484); RTN4(57142); SFXN5(94097); |
| | | TGOLN2(10618); TBC1D8(11138); ANAPC1(64682); IL1B(3553); BIN1 |
| | | (274); WDR33(55339); MCM6(4175); IFIH1(64135); STAT1(6772); FZD5 |
| | | (7855); TIGD1(200765); HDAC4(9759); FANCD2(2177); TATDN2(9797); |
| | | NR2C2(7182); PDCD6IP(10015); ARPP-21(10777); ZNF621(285268); LARS2 |
| | | (23395); IL17RB(55540); APPL(26060); C3orf37(56941); USP13(8975); |
| | | DVL3(1857); PSMD2(5708); SENP2(59343); TP73L(8626); SRGAP3(9901); |
| | | LOC401052(401052); RAF1(5894); MRPS25(64432); ZFYVE20(64145); |
| | | ZNF445(353274); IHPK2(51447); GPX1(2876); NEK4(6787); TMEM110 |
| | | (375346); FOXP1(27086); MAK3(80218); STAG1(10274); ATR(545); SIAH2 |
| | | (6478); EIF5A2(56648); PCGF3(10336); HD(3064); RGS12(6002); CD38 |
| | | (952); ANAPC4(29945); IL8(3576); CCNG2(901); FGF5(2250); SPP1(6696); |
| | | NFKB1(4790); FGF2(2247); PLK4(10733); CCRN4L(25819); SLC26A1 |
| | | (10861); LETM1(3954); APBB2(323); LEF1(51176); FBXW7(55294); NEK1 |
| | | (4750); CCT5(22948); ERBB2IP(55914); CCNB1(891); F2R(2149); RHOBTB3 |
| | | (22836); WDR36(134430); DCP2(167227); ARHGAP26(23092); SLC36A1 |
| | | (206358); UNC5A(90249); SEMA5A(9037); PRLR(5618); RICTOR(253260); |
| | | UNG2(10309); MCC(4163); CDC23(8697); NRG2(9542); HDAC3 |
| | | (8841); CAMK2A(815); CD74(972); FBXW11(23291); RNF130(55819); PHACTR1 |
| | | (221692); LTA(4049); CSNK2B(1460); PACSIN1(29993); PARC(23113); |
| | | SLC22A7(10864); FOXO3A(2309); TCF21(6943); RP5-875H10.1(389432); |
| | | C6orf71(389434); AKAP12(9590); C6orf62(81688); DHX16(8449); |
| | | BAK1(578); C6orf106(64771); CCND3(896); CUL7(9820); CLIC5(53405); |
| | | TNFRSF21(27242); TRAM2(9697); DST(667); MAP3K5(4217); PLAGL1 |
| | | (5325); KATNA1(11104); LATS1(9113); LFNG(3955); HDAC9(9734); C7orf16 |
| | | (10842); ADCY1(107); EGFR(1956); FZD9(8326); RINT1(60561); HBP1 |
| | | (26959); KIAA0828(23382); CASP2(835); CNTNAP2(26047); NOM1(64434); |
| | | SNX8(29886); TRIAD3(54476); PMS2(5395); CYCS(54205); JAZF1(221895); |
| | | IGFBP3(3486); YWHAG(7532); MCM7(4176); MTPN(136319); FASTK |
| | | (10922); ABCF2(10061); TNKS(8658); POLR3D(661); TERF1(7013); PI15 |
| | | (51050); CRISPLD1(83690); ATP6V1C1(528); RHPN1(114822); CTSB |
| | | (1508); BIN3(55909); TNFRSF10B(8795); TNFRSF10D(8793); TNFRSF10A |
| | | (8797); ENTPD4(9583); DUSP4(1846); BRF2(55290); WHSC1L1(54904); |
| | | FGFR1(2260); REXO1L1(254958); EXT1(2131); NUDT2(318); UNC13B |
| | | (10497); RECK(8434); MAK10(60560); DAPK1(1612); C9orf47(286223); |

Table 4-138

GADD45G(10912); TGFBR1(7046); INVS(27130); CDK9(1025); C9orf7(11094);
LCN12(286256); C9orf25(203259); FANCC(2176); PTCH(5727); ANKS6
(203286); TRAF1(7185); STRBP(55342); GOLGA1(2800); TSC1(7248);
SURF5(6837); BRD3(8019); ANAPC2(29882); HSPA14(51182); UNC5B
(219699); SEC24C(9632); KIAA1128(54462); KIF11(3832); SCD(6319);
BTRC(8945); PDCD11(22984); TCF7L2(6934); DOCK1(1793); MTG1(92170);
ABI1(10006); NRP1(8829); PARD3(56288); FZD8(8325); AP3M1(26985);
DUSP13(51207); DLG5(9231); HPS1(3257); FGF8(2253); ACTR1A(10121);
SMNDC1(10285); C10orf46(143384); MKI67(4288); STIM1(6786);
WEE1(7465); HIPK3(10114); API5(8539); EXT2(2132); INCENP(3619); SIPA1
(6494); LRP5(4041); IGHMBP2(3508); TPCN2(219931); CCND1(595);
BIRC3(330); BIRC2(329); CD3E(916); ABCG4(64137); LOC89944(89944);
HRAS(3265); MUCDHL(53841); IGF2(3481); RHOG(391); PTH(5741);
WT1(7490); CD59(966); CDCA5(113130); MAP3K11(4296); AYP1(84153);
CHKA(1119); MAP6(4135); PAK1(5058); BCL9L(283149); FEZ1(9638); SNX19
(399979); CCND2(894); CNAP1(9918); PTMS(5763); IAPP(3375); TEGT
(7009); MDM2(4193); CCT2(10576); TMTC3(160418); TCF1(6927);
MLXIP(22877); FZD10(11211); DUSP16(80824); LMO3(55885); TMTC1
(83857); MCRS1(10445); FAIM2(23017); KRT2B(51350); DDIT3(1649); CTDSP2
(10106); EEA1(8411); PPP1CC(5501); ATXN2(6311); HRK(8739); ANAPC5
(51433); MPHOSPH9(10198); SCARB1(949); EPIM(2054); RFC3
(5983); TFDP1(7027); LATS2(26524); FLJ25477(219287); KATNAL1(84056);
BCL2L2(599); C14orf35(122830); PSEN1(5663); DLST(1743); VASH1
(22846); NRXN3(9369); SLC24A4(123041); KIAA0125(9834); STRN3(29966);
CDKL1(8814); DLG7(9787); KIAA0831(22863); RAB15(376267); ERH
(2079); DPF3(8110); LTBP2(4053); PGF(5228); ZDHHC22(283576); JAG2
(3714); GOLGA8E(390535); GOLGA8G(283768); EIF3S1(8669); RAB11A
(8766); CSK(1445); LOC440295(440295); ALPK3(57538); FURIN(5045);
IGF1R(3480); ASB7(140460); NDN(4692); AVEN(57099); SLC12A6(9990);
SUHW4(54816); CSNK1G1(53944); UACA(55075); GRAMD2(196996);
MESDC2(23184); FLJ36208(283948); KIAA1924(197335); TSC2(7249);
CCNF(899); PDPK1(5170); DNAJA3(9093); NUBP1(4682); CARD15(64127);
CYLD(1540); KATNB1(10300); NOL3(8996); E2F4(1874); ELMO3(79767);
NFATC3(4775); CRISPLD2(83716); CDT1(81620); PKMYT1(9088);
ADCY9(115); GSPT1(2935); ERN2(10595); SEPT1(1731); N4BP1(9683); FTS
(64400); NUDT21(11051); CMTM4(146223); SLC12A4(6560); SMPD3
(55512); HSDL1(83693); PAFAH1B1(5048); TNFSF12(8742); MYOCD(93649);
TTC19(54902); SARM1(23098); NF1(4763); RAB11FIP4(84440); CDK5R1
(8851); LIG3(3980); TADA2L(6871); SOCS7(30837); STARD3(10948);
FZD2(2535); MAPT(4137); NXPH3(11248); MSI2(124540); PPM1D(8493);
TLK2(11011); SLC9A3R1(9368); LLGL2(3993); CDK3(1018); SPHK1
(8877); BIRC5(332); ENPP7(339221); CARKL(23729); P2RX1(5023); UNQ5783
(388325); AURKB(9212); RAD51L3(5892); NLE1(54475); PIP5K2B
(8396); PLXDC1(57125); ANKRD40(91369); SEPT4(5414); PTRH2(51651);
RECQL5(9400); WBP2(23558); C17orf70(80233); MAFG(4097); DCC(1630);
ESCO1(114799); ZNF24(7572); MBD1(4152); MBP(4155); TXNL4A(10907);
PARD6G(84552); CDC34(997); FSTL3(10272); GRIN3B(116444);
MUM1(84939); ZFR1(51343); CHAF1A(10036); UHRF1(29128); PIN1(5300);
DNM2(1785); IER2(9592); PIK3R2(5296); UBA52(7311); LOC115648
(115648); ZNF507(22847); MGC13096(84306); APLP1(333); MAP3K10(4294);
ARHGEF1(9138); APOE(348); FOSB(2354); GRLF1(2909); ATF6(22809);
NALP2(55655); AURKC(6795); C19orf26(255057); DEDD2(162989);
ERF(2077); PPP1R13L(10848); BBC3(27113); TRIB3(57761); BTBD3(22903);
KIF3B(9371); CEP250(11190); C20orf4(25980); CTNNBL1(56259);
LOC128439(128439); CD40(958); SLC2A10(81031); DNAJC5(80331); CSNK2A1
(1457); RNF24(11237); SCAND1(51282); KCNB1(3745); DIDO1(11083);
GM632(57473); SON(6651); ITSN1(6453); PDXK(8566); COL6A2(1292);
APP(351); BRWD1(54014); PLAC4(191585); SNF1LK(150094); ICOSLG
(23308); ITGB2(3689); PEX26(55670); SEPT5(5413); P2RXL1(9127);
LOC400891(400891); SMARCB1(6598); ADORA2A(135); HMG2L1(10042);
TRIOBP(11078); GTPBP1(9567); EP300(2033); SEPT3(55964); BIK(638);
ADM2(79924); BID(637); HTF9C(27037); MAPK1(5594); C1QTNF6(114904);
SSTR3(6753); CARD10(29775); CBX6(23466); LDOC1L(84247); CERK
(64781); MAPK12(6300); CHKB(1120); XK(7504); HDAC6(10013); OGT
(8473); NGFRAP1(27018); BIRC4(331); RP6-213H19.1(51765); AFF2(

Table 4-139

| | | |
|---|---|---|
| | | 2334); DKC1(1736); FUNDC2(65991); FIGF(2277); HCFC1(3054); MECP2 |
| | | (4204); DAZ2(57055); |
| hsa-miR-637 | 2104; | SAMD11(148398); PRDM16(63976); TP73(7161); HES3(390992); TNFRSF1B |
| | | (7133); PAX7(5081); PQLC2(54896); NBL1(4681); PLA2G2F(64600); |
| | | CCDC21(64793); GPR3(2827); SESN2(83667); RCC1(1104); KIAA1522(57648); |
| | | NCDN(23154); MACF1(23499); NFYC(4802); CDC20(991); PLK3(1263); |
| | | CDC14A(8556); BCL9(607); BNIPL(149428); C1orf60(65123); DAP3 |
| | | (7818); LMNA(4000); IGSF4B(57863); PCTK3(5129); MAPKAPK2(9261); |
| | | TGFB2(7042); TNFRSF18(8784); CPSF3L(54973); CDC2L2(985); TNFRSF14 |
| | | (8764); PLEKHG5(57449); FRAP1(2475); MAD2L2(10459); HSPG2 |
| | | (3339); E2F2(1870); RUNX3(864); SDC3(9672); MUTYH(4595); TESK2(10420); |
| | | FAF1(11124); BCL10(8915); NBPF4(148545); NOTCH2(4853); PYGO2 |
| | | (90780); DEDD(9191); MPZ(4359); MAPRE3(22924); KHK(3795); BIRC6 |
| | | (57448); SMYD5(10322); CCT7(10574); HTRA2(27429); HK2(3099); TCF7L1 |
| | | (83439); KCNIP3(30818); PROC(5624); RIF1(55183); CASP10(843); |
| | | CTDSP1(58190); COI4A3(1285); ITM2C(81618); PSMD1(5707); TMEM16G |
| | | (50636); E2F6(1876); CGREF1(10669); RTN4(57142); ADRA2B(151); |
| | | TMEM127(55654); ANAPC1(64682); PAX8(7849); BIN1(274); ERCC3(2071); |
| | | HS6ST1(9394); FZD5(7855); PAX3(5077); CUL3(8452); KIF1A(547); PCAF |
| | | (8850); PDCD6IP(10015); STAC(6769); EEFSEC(60678); ACPP(55); |
| | | DVL3(1857); TADA3L(10474); CIDEC(63924); TRAIP(10293); RASSF1(11186); |
| | | TUSC4(10641); CACNA2D2(9254); DCP1A(55802); RAB43(339122); |
| | | LRRC15(131578); MUC4(4585); WHSC1(7468); HD(3064); SORCS2(57537); |
| | | FLJ35725(152992); LETM1(3954); AFAP(60312); UNC5C(8633); SFRP2 |
| | | (6423); KIAA1909(153478); SKP2(6502); MRPS30(10884); ERBB2IP |
| | | (55914); RAD17(5884); CSNK1G3(1456); TCF7(6932); GALNT10(55568); |
| | | CPLX2(10814); UNC5A(90249); N4BP3(23138); TPPP(11076); SLC12A7 |
| | | (10723); PRLR(5618); UNG2(10309); MCC(4163); SEMA6A(57556); PPP2CA |
| | | (5515); NRG2(9542); FCHSD1(89848); FGF1(2246); PDGFRB(5159); |
| | | CD74(972); RNF130(55819); TCF19(6941); TNF(7124); AIF1(199); SYNGAP1 |
| | | (8831); SCUBE3(222663); MAPK13(5603); CDKN1A(1026); PARC(23113); |
| | | TTBK1(84630); VEGF(7422); BAG2(9532); MYO6(4646); TTK(7272); |
| | | TNFAIP3(7128); SASH1(23328); NEDD9(4739); MDGA1(266727); CUL7 |
| | | (9820); TRAM2(9697); DST(667); PLAGL1(5325); LATS1(9113); AGPAT4 |
| | | (56895); PDCD2(5134); CYP2W1(54905); AHR(196); SEPT7(989); EGFR |
| | | (1956); STAG3(10734); BCAP29(55973); TRIAD3(54476); PMS2(5395); |
| | | CARD4(10392); ELMO1(9844); CDK6(1021); PTCD1(26024); MCM7(4176); |
| | | LRCH4(4034); PERQ1(64599); EPHB4(2050); RASA4(10156); ZC3HC1 |
| | | (51530); HIPK2(28996); KIAA0773(9715); BMP1(649); POLR3D(661); TNFRSF10C |
| | | (8794); PTK2B(2185); ZNF703(80139); BAG4(9530); TACC1 |
| | | (6867); FZD6(8323); MYC(4609); WISP1(8840); LZTS1(11178); BIN3(55909); |
| | | TNFRSF10B(8795); ADRA1A(148); CLU(1191); KIF13B(23303); DUSP4 |
| | | (1846); PAG1(55824); RAD21(5885); MTSS1(9788); SCRT1(83482); ANKRD15 |
| | | (23189); UNC13B(10497); DAPK1(1612); C9orf47(286223); FGD3 |
| | | (89846); INVS(27130); ABL1(25); ADAMTSL2(9719); GRIN1(2902); IFNW1 |
| | | (3467); TAF1L(138474); BAG1(573); FANCG(2189); SEMA4D(10507); |
| | | PTCH(5727); TNFSF8(944); TRAF1(7185); CIZ1(25792); FAM78A(286336); |
| | | TSC1(7248); CARD9(64170); C9orf140(89958); ANAPC2(29882); UNC5B |
| | | (219699); FRAT1(10023); LZTS2(84445); SUFU(51684); PDCD11(22984); |
| | | TCF7L2(6934); BAG3(9531); KNDC1(85442); FZD8(8325); SPOCK2(9806); |
| | | DLG5(9231); FRAT2(23401); SFRP5(6425); SEC31L2(25956); FBXW4 |
| | | (6468); SMNDC1(10285); C10orf46(143384); MKI67(4288); IFITM1(8519); |
| | | WEE1(7465); HIPK3(10114); EXT2(2132); MDK(4192); C11orf11(747); |
| | | INCENP(3619); LGALS12(85329); SSSCA1(10534); PACS1(55690); CNIH2 |
| | | (254263); MGC33486(256472); RAD9A(5883); CABP4(57010); ATM |
| | | (472); TAGLN(6876); FLJ45300(399957); LOH11CR2A(4013); IRF7(3665); |
| | | MUCDHL(53841); SLC25A22(79751); IGF2(3481); CDKN1C(1028); RHOG |
| | | (391); APBB1(322); EIF4G2(1982); DKK3(27122); ABTB2(25841); TRAF6 |
| | | (7189); BAD(572); SYVN1(84447); MAP3K11(4296); BRMS1(25855); |
| | | CLCF1(23529); PPP1CA(5499); FGF4(2249); PAK1(5058); FZD4(8322); BTG4 |
| | | (54766); BCL9L(283149); CCND2(894); LTBR(4055); PTMS(5763); PTPN6 |
| | | (5777); BCL2L14(79370); TEGT(7009); NR4A1(3164); ESPL1(9700); |
| | | PDE1B(5153); CDK2(1017); FAM101A(144347); FZD10(11211); CACNA2D4 |
| | | (93589); ING4(51147); BCAT1(586); HDAC7A(51564); MCRS1(10445); |
| | | FAIM2(23017); C12orf22(81566); KRTHB5(3891); CDK4(1019); PAWR( |

Table 4-140

| | | |
|---|---|---|
| | | 5074); PCTK2(5128); DIP13B(55198); FAM109A(144717); ABCB9(23457); |
| | | CDK2AP1(8099); SCARB1(949); SUGT1(10910); FLJ40296(122183); ING1 |
| | | (3621); ATP11A(23250); TFDP1(7027); CSNK1A1L(122011); FOXO1A |
| | | (2308); PNN(5411); VASH1(22846); TMEM63C(57156); KIAA0329(9895); |
| | | TRAF3(7187); RNASE13(440163); CIDEB(27141); STRN3(29966); NFKBIA |
| | | (4792); LTBP2(4053); PGF(5228); NEK9(91754); PPP1R13B(23368); |
| | | AKT1(207); JAG2(3714); FLJ25222(374666); NDN(4692); BMF(90427); BCL2L10 |
| | | (10017); FLJ38723(255180); CSNK1G1(53944); PDCD7(10081); |
| | | KIAA1924(197335); MAPK8IP3(23162); TSC2(7249); CCNF(899); KREMEN2 |
| | | (79412); DNASE1(1773); GLIS2(84662); DNAJA3(9093); PLK1(5347); |
| | | CARD15(64127); GNAO1(2775); KATNB1(10300); MMP15(4324); WWP2 |
| | | (11060); CDT1(81620); CDK10(8558); AXIN(8312); CCDC78(124093); TPSG1 |
| | | (25823); NME3(4832); GSPT1(2935); ERN2(10595); P8(26471); C16orf58 |
| | | (64755); CMTM4(146223); TRADD(8717); LOC283849(283849); SLC12A4 |
| | | (6560); SMPD3(55512); PCOLN3(5119); HIC1(3090); ALOX12(239); |
| | | TNFSF12(8742); ALOX15B(247); MAPK7(5598); TRAF4(9618); RAB11 |
| | | FIP4(84440); LIG3(3980); LASP1(3927); RARA(5914); RND2(8153); MGC3123 |
| | | (79089); FZD2(2535); MAPT(4137); NGFR(4804); TLK2(11011); PRKCA |
| | | (5578); CDR2L(30850); CARD14(79092); BAIAP2(10458); GPS1(2873); |
| | | MNT(4335); P2RX1(5023); NALP1(22861); GPS2(2874); TP53(7157); AURKB |
| | | (9212); GAS7(8522); CORO6(84940); GAS2L2(246176); TBC1D3C(414060); |
| | | NR1D1(9572); BECN1(8678); BRCA1(672); DUSP3(1845); HDAC5 |
| | | (10014); PHB(5245); PPP1R9B(84687); BZRAP1(9256); ERN1(2081); USH1G |
| | | (124590); EXOC7(23265); FLJ45079(400624); SOCS3(9021); CBX4 |
| | | (8535); FLJ46026(400627); ARHGDIA(396); PCYT2(5833); CABLES1(91768); |
| | | MAPK4(5596); MALT1(10892); PHLPP(23239); NFATC1(4772); PARD6G |
| | | (84552); CDC34(997); MIDN(90007); APC2(10297); SF3A2(8175); GADD45B |
| | | (4616); FZR1(51343); HMG20B(10362); GIPC3(126326); CHAF1A |
| | | (10036); UHRF1(29128); MAP2K7(5609); PIN1(5300); DNM2(1785); CNN1 |
| | | (1264); NFIX(4784); F2RL3(9002); PIK3R2(5296); MGC26694(284439); |
| | | GATAD2A(54815); LOC148137(148137); MAP3K10(4294); AXL(558); ARHGEF1 |
| | | (9138); BCL3(602); APOE(348); KLC3(147700); FOSB(2354); HIF3A |
| | | (64344); GRLF1(2909); DHX34(9704); EHD2(30846); SPHK2(56848); |
| | | BAX(581); ATF5(22809); PPP2R1A(5518); LENG8(114823); AURKC(6795); |
| | | RNF126(55658); C19orf23(148046); TCF3(6929); BTBD2(55643); MKNK2 |
| | | (2872); MOBKL2A(126308); SGTA(6449); AES(166); ZBTB7A(51341); |
| | | PTPRS(5802); RANBP3(8498); ELL(8178); LASS1(10715); SF4(57794); |
| | | C19orf12(83636); FLJ36445(163183); DPF1(8193); PRX(57716); TGFB1 |
| | | (7040); POU2F2(5452); DEDD2(162989); ERF(2077); PPP1R13L(10848); |
| | | DMWD(1762); SLC8A2(6543); NAPA(8775); CGB7(94027); KLK4(9622); |
| | | KLK10(5655); SNPH(9751); CEP250(11190); CTNNBL1(56259); MYBL2 |
| | | (4605); KCNK15(60598); BIRC7(79444); RTEL1(51750); ZGPAT(84619); CSNK2A1 |
| | | (1457); FKBP1A(2280); BCL2L1(598); C20orf112(140688); E2F1 |
| | | (1880); PTPRT(11122); SYCP2(10388); CABLES2(81928); DIDO1(11083); |
| | | C20orf58(128414); CHRNA4(1137); GM632(57473); SON(6651); CHAF1B |
| | | (8208); KRTAP10-10(353333); SNF1LK(150094); C21orf58(54058); BCL2L13 |
| | | (23786); TSSK2(23617); CDC45L(8318); SEPT5(5413); MIF(4282); |
| | | GAS2L1(10634); CABP7(164633); MTP18(51537); MCM5(4174); GGA1(26088); |
| | | EP300(2033); SEPT3(55964); PPARA(5465); GTSE1(51512); DIP(23151); |
| | | MAPK1(5594); PPM1F(9647); MN1(4330); RBM9(23543); SSTR3(6753); |
| | | CARD10(29775); SOX10(6663); CBX6(23466); CBX7(23492); PDGFB |
| | | (5155); MAPK12(6300); CHKB(1120); SHOX(6473); PCTK1(5127); HDAC6 |
| | | (10013); MLLT7(4303); NLGN3(54413); TAF1(6872); CD40LG(959); PNMA3 |
| | | (29944); IQSEC2(23096); SEPT6(23157); CUL4B(8450); ARHGEF6 |
| | | (9459); HCFC1(3054); MECp2(4204); |
| hsa-miR-648 | 2105; | SAMD11(148398); PRKCZ(5590); TP73(7161); DFFB(1677); DNAJC16(23341); |
| | | CDC42(998); SFN(2810); SESN2(83667); KHDRBS1(10657); TXLNA |
| | | (200081); MACF1(23499); NFYC(4802); GADD45A(1647); SH3GLB1 |
| | | (51100); CDC7(8317); CDC14A(8556); SLC30A7(148867); SYCP1(8847); BCL9 |
| | | (607); BNIPL(149428); DAP3(7818); PEA15(8682); UHMK1(127933); |
| | | MPZL1(9019); CEP350(9857); RGS2(5997); PPP1R12B(4660); MDM4(4194); |
| | | IL24(11009); TRAF5(7188); CIAS1(114548); TNFRSF18(8784); CCNL2 |
| | | (81669); CDC2L2(985); TNFRSF14(8764); TNFRSF9(3604); FRAP1(2475); |
| | | MAD2L2(10459); E2F2(1870); TTMB(399474); KIAA0319L(79932); |
| | | MYCL1(4810); TESK2(10420); FAF1(11124); GFI1(2672); NOTCH2(4853 |

Table 4-141

); MCL1(4170); TPM3(7170); DEDD(9191); ASPM(259266); KIF21B(23046);
TP53BP2(7159); FOSL2(2355); BIRC6(57448); PRKCE(5581); CCT7(10574);
HTRA2(27429); HK2(3099); FLJ20758(55037); KCNIP3(30818); BRRN1
(23397); FLJ13096(80067); ZAK(51776); CASP10(843); CASP8(841);
CD28(940); CGREF1(10669); PPM1G(5496); RTN4(57142); TIA1(7072);
EDAR(10913); ANAPC1(64682); BIN1(274); IFIH1(64135); TLK1(9874);
STAT1(6772); CLK1(1195); BARD1(580); CUL3(8452); HDAC4(9759); FANCD2
(2177); DLEC1(9940); CTNNB1(1499); RBM5(10181); APPL(26060);
PCNP(57092); EAF2(55840); MCM2(4171); NEK11(79858); FXR1(8087);
PSMD2(5708); SENP2(59343); TADA3L(10474); CIDEC(63924); RAF1(5894);
AXUD1(64651); KIAA1143(57456); RASSF1(11186); NEK4(6787); IL17RD
(54756); UBE1C(9039); GSK3B(2932); STAG1(10274); TFDP2(7029);
SIAH2(6478); SH3BP2(6452); HD(3064); HCAP-G(64151); NFKB1(4790);
FGF2(2247); CCRN4L(25819); APBB2(323); LEF1(51176); ANAPC10
(10393); SFRP2(6423); NEK1(4750); MFAP3L(9848); SKP2(6502); NIPBL
(25836); CCNB1(891); F2R(2149); APC(324); TCF7(6932); SLC36A1(206358);
UNC5A(90249); PRLR(5618); UNG2(10309); MCC(4163); SEMA6A
(57556); NME5(8382); HDAC3(8841); FGF1(2246); CSNK1A1(1452); C5orf4
(10826); SLIT3(6586); FBXW11(23291); DUSP1(1843); DDX41(51428);
RIPK1(8737); LY86(9450); E2F3(1871); ANKS1A(23294); MAPK14(1432);
MAPK13(5603); PARC(23113); TTK(7272); FOXO3A(2309); TNFAIP3(7128);
SASH1(23328); NEDD9(4739); GABBR1(2550); MDC1(9656); DAXX
(1616); IHPK3(117283); CCND3(896); SUPT3H(8464); TNFRSF21(27242);
DST(667); FRK(2444); MAP3K5(4217); PLAGL1(5325); KATNA1(11104);
LATS1(9113); FOXK1(221937); STK17A(9263); EGFR(1956); FZD9(8326);
FZD1(8321); STAG3(10734); HBP1(26959); BCAP29(55973); CUL1(8454);
TRIAD3(54476); CYCS(54205); SCRN1(9805); CARD4(10392); YWHAG
(7532); CDK6(1021); MCM7(4176); FIS1(51024); BRAF(673); EPHA1
(2041); FASTK(10922); POLR3D(661); TNFRSF10C(8794); PTK2B(2185);
BAG4(9530); TACC1(6867); SULF1(23213); EBAG9(9166); MYC(4609);
WISP1(8840); KIAA1875(340390); PSD3(23362); LZTS1(11178); BIN3(55909);
TNFRSF10B(8795); TRIM35(23087); CLU(1191); DUSP4(1846); MOS
(4342); CCNE2(9134); RAD21(5885); EXT1(2131); TNFRSF11B(4982);
ANKRD15(23189); JAK2(3717); UBE2R2(54926); UNC13B(10497); NTRK2
(4915); DAPK1(1612); GADD45G(10912); FGD3(89846); INVS(27130);
C9orf28(89853); CDK9(1025); ABL1(25); IFNA2(3440); CDKN2A(1029);
CDKN2B(1030); FANCG(2189); GAS1(2619); SEMA4D(10507); TRAF1(7185);
CIZ1(25792); TSC1(7248); SURF4(6836); CARD9(64170); ANAPC2
(29882); ZMYND11(10771); ARL5B(221079); SGPL1(8879); UNC5B(219699);
CHST3(9469); TSPAN14(81619); LZTS2(84445); BTRC(8945); SUFU
(51684); PDCD11(22984); CSPG6(9126); BAG3(9531); DOCK1(1793); CUL2
(8453); C10orf72(196740); ZWINT(11130); DLG5(9231); SFRP5(6425);
FBXW4(6468); ARL3(403); VAX1(11023); C10orf46(143384); TIAL1(7073);
STIM1(6786); WEE1(7465); HIPK3(10114); API5(8539); F2(2147); INCENP
(3619); LGALS12(85329); VEGFB(7423); SSSCA1(10534); SIPA1(6494);
RPS6KB2(6199); LRP5(4041); CCND1(595); FADD(8772); PANX1(24145);
CUL5(8065); ATM(472); SNF1LK2(23235); LOH11CR2A(4013); SLC37A2
(219855); APBB1(322); EIF4G2(1982); DKK3(27122); SLC1A2(6506);
CDCA5(113130); RELA(5970); BRMS1(25855); PPP1CA(5499); PAK1(5058);
FZD4(8322); SESN3(143686); PPP2R1B(5519); BCL9L(283149); H2AFX
(3014); CCND2(894); CNAP1(9918); PTPN6(5777); BCL2L14(79370);
DDX11(1663); TEGT(7009); ESPL1(9700); CDK2(1017); APAF1(317); KIAA1853
(84530); KNTC1(9735); TNFRSF1A(7132); NOL1(4839); LALBA
(3906); MCRS1(10445); FAIM2(23017); C12orf22(81566); CDK4(1019); PAWR
(5074); DUSP6(1848); BTG1(694); DIP13B(55198); ATXN2(6311); ANAPC5
(51433); DIABLO(56616); CDK2AP1(8099); SCARB1(949); TNFRSF19
(55504); CDK8(1024); RFP2(10206); TFDP1(7027); LATS2(26524); LIG4
(3981); CDKN3(1033); PSEN1(5663); VASH1(22846); TRAF3(7187); KNS2
(3831); ACIN1(22985); CIDEB(27141); GZMH(2999); GZMB(3002); STRN3
(29966); CDKL1(8814); DLG7(9787); ERH(2079); NEK9(91754); TGFB3
(7043); BAG5(9529); PPP1R13B(23368); AKT1(207); JAG2(3714); MAPK6
(5597); RNF111(54778); CCNB2(9133); SNX1(6642); CSK(1445); ALPK3
(57538); ABHD2(11057); IGF1R(3480); ATP10A(57194); SLC12A6
(9990); BMF(90427); CSNK1G1(53944); TRAF7(84231); CCNF(899); PDPK1
(5170); DNASE1(1773); DNAJA3(9093); PLK1(5347); PAPD5(64282);

Table 4-142

| | | |
|---|---|---|
| | | CARD15(64127); NOL3(8996); E2F4(1874); ELMO3(79767); CTCF(10664); |
| | | CDT1(81620); CDK10(8558); AXIN1(8312); C16orf28(65259); NME3(4832); |
| | | PKMYT1(9088); GSPT1(2935); ERN2(10595); P8(26471); MAPK3(5595); |
| | | PYCARD(29108); DNAJA2(10294); CIAPIN1(57019); TK2(7084); APPBP1 |
| | | (8883); TRADD(8717); RANBP10(57610); SLC12A4(6560); SMPD3 |
| | | (55512); TXNL4B(54957); PCOLN3(5119); HIC1(3090); PAFAH1B1(5048); |
| | | ALOX12(239); TNFSF12(8742); ALOX15B(247); MAPK7(5598); NLK |
| | | (51701); TRAF4(9618); NF1(4763); RAB11FIP4(84440); LIG3(3980); STARD3 |
| | | (10948); MAPT(4137); ABCC3(8714); TLK2(11011); PRKCA(5578); SLC9A3R1 |
| | | (9368); CDK3(1018); BIRC5(332); ANAPC11(51529); MNT(4335); |
| | | P2RX1(5023); NALP1(22861); GPS2(2874); TP53(7157); AURKB(9212); |
| | | GAS7(8522); HCP1(113235); GAS2L2(246176); BRCA1(672); HDAC5(10014); |
| | | EFTUD2(9343); MPO(4353); CSNK1D(1453); MAPK4(5596); DCC(1630); |
| | | LPIN2(9663); ROCK1(6093); BCL2(596); PARD6G(84552); CDC34 |
| | | (997); CSNK1G2(1455); FZR1(51343); CHAF1A(10036); DNM2(1785); RAB8A |
| | | (4218); PIK3R2(5296); UBA52(7311); MAP3K10(4294); ARHGEF1(9138); |
| | | BCL3(602); APOE(348); BCL2L12(83596); ATF5(22809); NALP2(55655); |
| | | AURKC(6795); MATK(4145); DAPK3(1613); INSR(3643); CDKN2D |
| | | (1032); DPF1(8193); ERF(2077); ERCC2(2068); LIG1(3978); FLJ10922(55260); |
| | | KLK10(5655); MCM8(84515); PLCB1(23236); CEP250(11190); CD40 |
| | | (958); PARD6B(84612); VAPB(9217); BIRC7(79444); CSNK2A1(1457); BCL2L1 |
| | | (598); E2F1(1869); SCAND1(51282); RBL1(5933); CTCFL(140690); |
| | | CABLES2(81928); DIDO1(11083); USP16(10600); MX1(4599); BTG3(10950); |
| | | APP(351); ITGB2(3689); BCL2L13(23786); SEPT5(5413); MIF(4282); |
| | | ADORA2A(135); GAS2L1(10634); NF2(4771); YWHAH(7533); EP300(2033); |
| | | SEPT3(55964); FLJ20699(55020); GTSE1(51512); BID(637); MAPK1 |
| | | (5594); PPM1F(9647); LOC388886(388886); MN1(4330); CARD10(29775); |
| | | TOB2(10766); SMC1L2(27127); PCTK1(5127); HDAC6(10013); CCNB3 |
| | | (85417); MLLT7(4303); CXorf39(139231); AGTR2(186); STAG2(10735); |
| | | DKC1(1736); SMC1L1(8243); RNF12(51132); BTK(695); DCX(1641); SEPT6 |
| | | (23157); CUL4B(8450); PDCD8(9131); ARHGEF6(9459); BCAP31(10134); |
| | | HCFC1(3054); MECP2(4204); MTCP1(4515); |
| hsa-miR-647 | 2106; | TTLL10(254173); FAM79A(127262); TP73(7161); TNFRSF1B(7133); FLJ43806 |
| | | (399563); PAX7(5081); PLA2G2F(64600); CDC42(998); NUDC(10726); |
| | | SESN2(83667); RCC1(1104); EIF2C1(26523); MACF1(23499); CDC20 |
| | | (991); ST3GAL3(6487); GADD45A(1647); SH3GLB1(51100); LMO4(8543); |
| | | PROK1(84432); TTF2(8458); BCL9(607); FDPS(2224); IGSF4B(57863); |
| | | PEA15(8682); UHMK1(127933); QSCN6(5768); C1orf21(81563); PPP1R12B |
| | | (4660); PPFIA4(8497); ADORA1(134); IL19(29949); IL24(11009); C1orf95 |
| | | (375057); C1orf159(54991); TNFRSF18(8784); CCNL2(81669); TNFRSF14 |
| | | (8764); TNFRSF9(3604); FRAP1(2475); MTHFR(4524); PADI2(11240); |
| | | RCC2(55920); PLA2G2D(26279); WASF2(10163); PHC2(1912); KIAA0319L |
| | | (79932); CLSPN(63967); BMP8B(656); RIMS3(9783); HIVEP3(59269); |
| | | MUTYH(4595); TESK2(10420); PRDX1(5052); SLC1A7(6512); C1orf183 |
| | | (55924); NOTCH2(4853); POGZ(23126); SMG5(23381); ISG20L2(81875); |
| | | KCNJ10(3766); KIAA0040(9674); GLT25D2(23127); ASPM(259266); |
| | | KIF21B(23046); SYT2(127833); CHIT1(1118); ETNK2(55224); TMEM63A |
| | | (9725); PPP1CB(5500); LBH(81606); BIRC6(57448); PRKCE(5581); MSH2 |
| | | (4436); SPTBN1(6711); HTRA2(27429); HK2(3099); TCF7L1(83439); ATOH8 |
| | | (84913); KCNIP3(30818); BCL2L11(10018); CCNT2(905); RIF1(55183); |
| | | BBS5(129880); ZAK(51776); CASP10(843); ICOS(29851); ALS2CR19 |
| | | (117583); COL4A3(1285); PSMD1(5707); LOC339768(339768); BOK(666); |
| | | FLJ33534(285150); RBJ(51277); CGREF1(10669); ZNF513(130557); PPM1G |
| | | (5496); EIF2AK2(5610); PRKD3(23683); THADA(63892); FLJ40172 |
| | | (285051); RTN4(57142); CCT4(10575); LOC51057(51057); SPRED2(200734); |
| | | TIA1(7072); TGFA(7039); FLJ10081(55683); REV1L(51455); CREG2 |
| | | (200407); BUB1(699); ANAPC1(64682); IL1B(3553); ERCC3(2071); WDR33 |
| | | (55339); MCM6(4175); GTDC1(79712); TTN(7273); TRAK2(66008); BARD1 |
| | | (580); MARCH4(57574); IGFBP5(3488); PAX3(5077); C2orf19(394261); |
| | | PER2(8864); HDAC4(9759); FANCD2(2177); PCAF(8850); CCBP2(1238); |
| | | BSN(8927); HEMK1(51409); GNL3(26354); ABHD6(57406); ALCAM |
| | | (214); SLC35A5(55032); PLXNA1(5361); MCM2(4171); TP73L(8626); IL1RAP |
| | | (3556); SRGAP3(9901); ZCSL2(285381); KIAA1143(57456); PFKFB4 |
| | | (5210); QRICH1(54870); RASSF1(11186); PB1(55193); TMEM110(375346); |
| | | FAM107A(11170); DOC1(11259); GSK3B(2932); KPNA1(3836); MGLL |

Table 4-143

(11343); STAG1(10274); TNFSF10(8743); MASP1(5648); HD(3064); CPEB2
(132864); ANAPC4(29945); SEPT11(55752); FGF5(2250); NFKB1(4790);
TLR2(7097); STOX2(56977); SLC26A1(10861); TMEM129(92305); LETM1
(3954); MXD4(10608); APBB2(323); SRD5A2L2(253017); SNCA(6622);
UNC5C(8633); DKK2(27123); LEF1(51176); ANAPC10(10393); KIAA1909
(153478); NIPBL(25836); CARD6(84674); GZMA(3001); ERBB2IP(55914);
APC(324); TCF7(6932); FLJ41603(389337); SLC36A1(206358); DKFZP564I1171
(25845); DAP(1611); PRLR(5618); DAB2(1601); UNG2(10309);
LHFPL2(10184); SEMA6A(57556); MARCH3(115123); IRF1(3659); NME5
(8382); LRRTM2(26045); APBB3(10307); SLC36A2(153201); GABRB2
(2561); SH3PXD2B(285590); LOC51149(51149); RNF130(55819); RIPK1
(8737); C6orf151(154007); E2F3(1871); GMNN(51053); TCF19(6941);
ZFAND3(60685); TBN(129685); SRF(6722); PARC(23113); RUNX2(860);
PTP4A1(7803); FOXO3A(2309); SASH1(23328); ULBP1(80329); LOC401233
(401233); F13A1(2162); HLA-DOA(3111); CCND3(896); CUL7(9820);
MCM3(4172); TRAM2(9697); DST(667); GJB7(375519); ATG5(9474); PLAGL1
(5325); LATS1(9113); LFNG(3955); TTYH3(80727); WIPI2(26100); HDAC9
(9734); ADCY1(107); EGFR(1956); LANCL2(55915); FZD9(8326); ZNF655
(79027); STAG3(10734); CUTL1(1523); HBP1(26959); BCAP29(55973);
CUL1(8454); LOC155060(155060); LOC90639(90639); MAD1L1(8379);
PMS2(5395); ICA1(3382); SOSTDC1(25928); CRHR2(1395); CALN1
(83698); STX1A(6804); YWHAG(7532); BAIAP2L1(55971); SMURF1(57154);
PERQ1(64599); POLR2J2(246721); IMPDH1(3614); UBE2H(7328);
HIPK2(28996); DLGAP2(9228); ZNF705CP(389631); RAI16(64760); POLR3D
(661); PDLIM2(64236); PTK2B(2185); UNC5D(137970); BAG4(9530);
CHRAC1(54108); GML(2765); RHPN1(114822); C8orf13(83648); TNFRSF10B
(8795); CLU(1191); DUSP4(1846); FUT10(84750); FGFR1(2260); SFRP1
(6422); CEBPD(1052); PLAG1(5324); PDE7A(5150); NPAL2(79815);
EXT1(2131); LYNX1(66004); RP11-255A11.18(441459); MGC21881(389741);
NTRK2(4915); MAK10(60560); DAPK1(1612); INVS(27130); DAB2IP
(153090); CDK9(1025); SLC25A25(114789); SPTAN1(6709); USP20(10868);
RXRA(6256); KCNT1(57582); TRAF2(7186); PTGDS(5730); FLJ36268
(401563); CACNA1B(774); SMU1(55234); FANCG(2189); SHB(6461); APBA1
(320); GAS1(2619); SEMA4D(10507); PTCH(5727); C9orf97(158427);
TRIM14(9830); ANKS6(203286); TRAF1(7185); ENG(2022); CIZ1(25792);
TSC1(7248); ANAPC2(29882); ZMYND11(10771); RET(5979); LOC440558
(440558); CDC2(983); SGPL1(8879); UNC5B(219699); CHST3(9469); SFTPA1
(6435); PTEN(5728); KIF11(3832); NOLC1(9221); SUFU(51684); C10orf26
(54838); PDCD11(22984); ADRB1(153); BAG3(9531); DOCK1(1793);
C10orf39(282973); KNDC1(85442); IL2RA(3559); PIP5K2A(5305); CUL2
(8453); ARHGAP22(58504); C10orf72(196740); ZWINT(11130); DNAJB12
(54788); CAMK2G(818); DLG5(9231); SFTPA2(6436); MAT1A(4143);
C10orf13(143282); FRAT2(23401); SFRP5(6425); SEC31L2(25956); FGF8
(2253); FAM26C(255022); SH3PXD2A(9644); C10orf46(143384); FGFR2
(2263); GALNAC4S-6ST(51363); MKI67(4288); AP2A2(161); STIM1(6786);
GAS2(2620); HIPK3(10114); CRY2(1408); MDK(4192); F2(2147); DAK
(26007); C11orf11(747); INCENP(3619); SSSCA1(10534); SIPA1(6494); RPS6KB2
(6199); LRP5(4041); CCND1(595); BIRC3(330); ATM(472); CD3G
(917); LOH11CR2A(4013); CHEK1(1111); JAM3(83700); SIRT3(23410); DUSP8
(1850); IGF2(3481); RHOG(391); APBB1(322); WT1(7490); COMMD9
(29099); HRASLS3(11145); BAD(572); CDCA5(113130); SYVN1(84447);
MAP3K11(4296); BRMS1(25855); MTL5(9633); MRGPRF(219928); FZD4
(8322); SESN3(143686); CASP1(834); MGC33948(160140); ZW10(9183);
PCSK7(9159); SCN2B(6327); BCL9L(283149); H2AFX(3014); CCND2(894);
TNFRSF7(939); CNAP1(9918); PTMS(5763); LRRC23(10233); FOXJ2
(55810); FLJ20489(55652); PFKM(5213); ESPL1(9700); METTL7B(196410);
MDM2(4193); APAF1(317); OAS3(4940); FBXW8(26259); KIAA1853
(84530); KIAA0152(9761); TCF1(6927); KNTC1(9735); HIP1R(9026); FZD10
(11211); NOL1(4839); ING4(51147); LRP6(4040); MGC24039(160518);
P11(8909); LALBA(3906); MCRS1(10445); FAIM2(23017); C12orf22(81566);
GLS2(27165); PTPRB(5787); ARPC3(10094); PPP1CC(5501); MPHOSPH9
(10198); TNFRSF19(55504); ATP8A2(51761); BRCA2(675); WDFY2
(115825); LOC440138(440138); ING1(3621); TFDP1(7027); GAS6(2621); LATS2
(26524); SLC7A1(6541); FOXO1A(2308); KLF12(11278); LOC400145
(400145); STK24(8428); TXNDC(81542); C14orf108(55745); GALNTL1(

Table 4-144

| | | |
|---|---|---|
| | | 57452); RGS6(9628); VASH1(22846); SLC24A4(123041); TRAF3(7187); TNFAIP2 |
| | | (7127); KNS2(3831); ACIN1(22985); IPO4(79711); GZMH(2999); |
| | | STRN3(29966); SPTB(6710); DPF3(8110); LTBP2(4053); NEK9(91754); CDC42BPB |
| | | (9578); BAG5(9529); JAG2(3714); APBA2(321); PAK6(56924); ZNF609 |
| | | (23060); KIF23(9493); PML(5371); CSK(1445); PDE8A(5151); AKAP13 |
| | | (11214); FURIN(5045); IGF1R(3480); SLC12A6(9990); BMF(90427); |
| | | DAPK2(23604); CSNK1G1(53944); CLN6(54982); GRAMD2(106996); STRA6 |
| | | (64220); ACSBG1(23205); RAB40C(57799); CRAMP1L(57585); GFER |
| | | (2671); TSC2(7249); TRAF7(84231); E4F1(1877); DNASE1(1773); ABCC1 |
| | | (4363); TNT(162083); MAZ(4150); RNF40(9810); GPT2(84706); GNAO1 |
| | | (2775); CX3CL1(6376); KATNB1(10300); ELMO3(79767); CTCF(10664); KIAA0513 |
| | | (9764); NHN1(124245); FLJ45121(400556); LOC197322(197322); |
| | | RPL13(6137); CCDC78(124093); PKMYT1(9088); C16orf5(29965); GSPT1 |
| | | (2935); ERN2(10505); P8(26471); PYCARD(29108); DNAJA2(10294); |
| | | FTS(64400); TK2(7084); CMTM4(146223); TRADD(8717); SLC12A4(6560); |
| | | SMPD3(55512); TXNL4B(54957); CHST6(4166); SLC7A5(8140); PCOLN3 |
| | | (5119); HIC1(3090); KIF1C(10749); ALOX12(239); ARHGEF15(22899); |
| | | NTN1(9423); ALKBH5(54890); SLC5A10(125206); MAPK7(5598); SPECC1 |
| | | (92521); NLK(51701); SARM1(23098); GOSR1(9527); NF1(4763); RHBDL3 |
| | | (162494); CCL2(6347); LIG3(3980); FZD2(2535); MAPT(4137); GOSR2 |
| | | (9570); PNPO(55163); ABCC3(8714); ACE(1636); WDR68(10238); |
| | | MAP3K3(4215); PRKCA(5578); SSTR2(6752); SPHK1(8877); CARD14(79092); |
| | | CHMP6(79643); GPS1(2873); MNT(4335); CARKL(23729); CAMKK1 |
| | | (84254); UNQ5783(388325); NALP1(22861); GPS2(2874); TOM1L2(146691); |
| | | FLOT2(2319); FLJ31952(146857); GAS2L2(246176); ORMDL3(94103); |
| | | BECN1(8678); BRCA1(672); HDAC5(10014); PLEKHM1(9842); CA10 |
| | | (56934); CUEDC1(404093); MPO(4353); SEPT4(5414); APPBP2(10513); |
| | | ERN1(2081); RECQL5(9400); SOCS3(9021); TBC1D16(125058); ARHGDIA |
| | | (396); PCYT2(5833); CIDEA(1149); C18orf1(753); CABLES1(91768); |
| | | MAPRE2(10982); STATIP1(55250); SETBP1(26040); LIPG(9388); DCC(1630); |
| | | MALT1(10892); LPIN2(9663); MBP(4155); CIRBP(1153); APC2(10297); |
| | | CSNK1G2(1455); FZR1(51343); HMG20B(10362); CHAF1A(10036); |
| | | TNFSF9(8744); PPAN(56342); P2RY11(5032); DNM2(1785); NFIX(4784); |
| | | RAB8A(4218); GLT25D1(79709); PIK3R2(5296); SSBP4(170463); CRTC1 |
| | | (22373); MGC26694(284439); CCNE1(898); KCTD15(79047); SAMD4B(55095); |
| | | MAP3K10(4294); AXL(558); PVR(5817); APOE(348); FOSB(2354); |
| | | ATF5(22809); PPP2R1A(5518); AURKC(6795); C19orf6(91304); DAPK3 |
| | | (1613); PLAC2(257000); RFX2(5990); LPHN1(22859); DNAJB1(3337); SIRT2 |
| | | (22933); FBXO17(115290); TGFB1(7040); PPP1R13L(10848); DMWD |
| | | (1762); BBC3(27113); SOX12(6666); STK35(140901); PRND(23627); MYLK2 |
| | | (85366); POFUT1(23509); CEP250(11190); MYBL2(4605); CSE1L(1434); |
| | | PARD6B(84612); VAPB(9217); STX16(8675); ARFGAP1(55738); C20orf149 |
| | | (79144); ZGPAT(84619); DNAJC5(80331); RASSF2(9770); SLC23A2 |
| | | (9962); BCL2L1(598); E2F1(1869); C20orf117(140710); PTPRT(11122); PLTP |
| | | (5360); ELMO2(63916); KCNB1(3745); UBE2V1(7335); NFATC2(4773); |
| | | CTCFL(140690); LAMA5(3911); CABLES2(81928); FLJ30313(253868); |
| | | DIDO1(11083); STMN3(50861); GM632(57473); MX1(4599); APP(351); DSCR3 |
| | | (10311); SNF1LK(150094); PTTG1IP(754); ITGB2(3689); C21orf93 |
| | | (246704); S100B(6285); BCL2L13(23786); CDC45L(8318); UBE2L3(7332); |
| | | ADORA2A(135); KREMEN1(83999); GAS2L1(10634); MCM5(4174); PSCD4 |
| | | (27128); CACNA1I(8911); EP300(2033); ZC3H7B(23264); SEPT3(55964); |
| | | FAM109B(150368); FBLN1(2192); MAPK8IP2(23542); BID(637); LOC128977 |
| | | (128977); MAPK1(5594); PPM1F(9647); ZNF70(7621); MN1(4330); |
| | | SSTR3(6753); TOB2(10766); NHP2L1(4809); POLDIP3(84271); LDOC1L |
| | | (84247); C22orf9(23313); TBL1X(6907); HDAC6(10013); CCNB3(85417); |
| | | RP1-112K5.2(90121); MLLT7(4303); TAF1(6872); PNMA3(29944); BGN |
| | | (633); ATP2B3(492); AVPR2(554); IKBKG(8517); FOXP3(50943); SMC1L1 |
| | | (8243); CUL4B(8450); PDCD8(9131); FAM58A(92002); HCFC1(3054); |
| hsa-miR-648 | 2107; | H6PD(9563); ZBTB40(9923); SESN2(83667); PTPRU(10076); MACF1(23499); |
| | | NFYC(4802); LY9(4063); RASAL2(9462); C1orf21(81563); ATP2B4 |
| | | (493); SRGAP2(23380); TGFB2(7042); MARK1(4139); TNFRSF9(3604); CTNNBIP1 |
| | | (56998); KIAA0319L(79932); TBX15(6913); NOTCH2(4853); MEF2D |
| | | (4209); CD84(8832); TP53BP2(7159); LPIN1(23175); CAD(790); FLJ46838 |
| | | (440885); ANTXR1(84168); HTRA2(27429); TCF7L1(83439); KCN |

Table 4-145

| | | |
|---|---|---|
| | | IP3(30818); NPAS2(4862); TSN(7247); GPD2(2820); CFLAR(8837); COL4A3 |
| | | (1285); EIF2AK2(5610); TLK1(9874); NCKAP1(10787); HDAC4(9759); |
| | | FANCD2(2177); PCAF(8850); RBM5(10181); MBNL1(4154); OPA1(4976); |
| | | TADA3L(10474); AXUD1(64651); RASSF1(11186); FHIT(2272); SIAH2 |
| | | (6478); DGKG(1608); HD(3064); CPEB2(132864); LOC285429(285429); FGF5 |
| | | (2250); STOX2(56977); MLR1(254251); MGC16169(93627); SFRP2(6423); |
| | | LOC90624(90624); IL13(3596); TCF7(6932); TPPP(11076); PRLR(5618); |
| | | CD14(929); FGF1(2246); FBXW11(23291); MRS2L(57380); MOG(4340); |
| | | PARC(23113); TNFAIP3(7128); PHACTR2(9749); SASH1(23328); QKI |
| | | (9444); TNFRSF21(27242); DST(667); BVES(11149); FRK(2444); SGK(6446); |
| | | MAP3K5(4217); EGFR(1956); LOC441257(441257); ASB4(51666); STAG3 |
| | | (10734); HBP1(26959); INHBA(3624); NUDCD3(23386); CALN1(83698); |
| | | HIPK2(28996); CHMP7(91782); SPFH2(11160); TACC1(6867); GML |
| | | (2765); OPRK1(4986); PLAG1(5324); RAD21(5885); ANKRD15(23189); |
| | | CREB3(10488); NTRK2(4915); RP11-19J3.3(401541); TMOD1(7111); GPR107 |
| | | (57720); LAMC3(10319); KCNT1(57582); LCN12(286256); LOC613206 |
| | | (613206); WDR37(22884); CHST3(9469); LDB3(11155); FAS(355); KIF11 |
| | | (3832); BTRC(8945); PTPRE(5791); AKR1C2(1646); RASGEF1A(221002); |
| | | SH3PXD2A(9644); C10orf46(143384); MRI67(4288); FLJ46300(399827); |
| | | TUB(7275); ZFP91(80829); CUL5(8065); IGF2(3481); WT1(7490); PAK1 |
| | | (5058); ZW10(9183); CCND2(894); TNFRSF7(939); PTMS(5763); PTPN6 |
| | | (5777); CDKN1B(1027); KCNMB4(27345); TMEM132B(114795); C12orf53 |
| | | (196500); GRIN2B(2904); SLC2A13(114134); FAIM2(23017); PHLDA1 |
| | | (22822); PAWR(5074); BTG1(694); TMCC3(57458); DIP13B(55198); ANAPC7 |
| | | (51434); PPP1CC(5501); DIABLO(56616); SCARB1(949); ATP8A2 |
| | | (51761); LATS2(26524); DCAMKL1(9201); FOXO1A(2308); BCL2L2(599); |
| | | RNF31(55072); ADCK1(57143); KIAA0329(9895); TRAF3(7187); ACIN1 |
| | | (22985); NFKBIA(4792); NEK9(91754); CHES1(1112); BTBD7(55727); BUB1B |
| | | (701); RNF111(54778); CCNB2(9133); NDN(4692); BNIP2(663); CSNK1G1 |
| | | (53944); CALML4(91860); TLE3(7090); TSC2(7249); CCNF(899); CARD15 |
| | | (64127); KATNB1(10300); ELMO3(79767); CYB5B(80777); NFAT5 |
| | | (10725); WWP2(11060); MON1B(22879); FLJ45121(400556); NME3(4832); |
| | | SMG1(23049); ERN2(10595); RANBP10(57610); SLC12A4(6560); TNFSF12 |
| | | (8742); MAPK7(5598); MAP2K3(5606); NLK(51701); TRAF4(9618); |
| | | LASP1(3927); PRKCA(5578); CARD14(79092); MNT(4335); STAT3(6774); |
| | | ERN1(2081); EXOC7(23265); CABLES1(91768); MALT1(10892); DYM |
| | | (54308); PARD6G(84552); DAPK3(1613); RANBP3(8498); ZNF44(51710); |
| | | MGC71805(403113); ZNF443(10224); DNASE2(1777); PLCB1(23236); HM13 |
| | | (81502); EPB41L1(2036); SLC23A2(9962); SCAND1(51282); SLA2(84174); |
| | | SYCP2(10388); LAMA5(3911); MX1(4599); HLCS(3141); PLAC4(191585); |
| | | UBE2L3(7332); C22orf15(150248); APOL6(80830); EP300(2033); |
| | | MAPK1(5594); IL2RB(3560); CARD10(29775); PDGFB(5155); MAPK12 |
| | | (6300); DIAPH2(1730); RNF12(51132); CUL4B(8450); XX-FW81657B9.4 |
| | | (8270); |
| hsa-miR-650 | 2108; | SAMD11(148398); PRKCZ(5590); TP73(7161); C1orf188(148646); H6PD |
| | | (9563); PEX14(5195); NBL1(4681); LDLRAP1(26119); SFN(2810); SESN2 |
| | | (83667); KHDRBS1(10657); RBBP4(5928); MACF1(23499); SH3GLB1(51100); |
| | | CDC14A(8556); RAP1A(5906); BCL9(607); UHMX1(127933); FASLG |
| | | (356); C1orf21(81563); IL19(29949); IL24(11009); G0S2(50486); TRAF5 |
| | | (7188); CIAS1(114548); CCNL2(81669); TNFRSF14(8764); PLEKEIG5(57449); |
| | | TNFRSF9(3604); CTNNBIP1(56998); FRAP1(2475); MAD2L2(10459); |
| | | C1orf166(79594); RAP1GA1(5909); E2F2(1870); WASF2(10163); SDC3 |
| | | (9672); CLSPN(63967); MUTYH(4595); TESK2(10420); FAF1(11124); BCL10 |
| | | (8915); GFI1(2672); NOTCH2(4853); THEM5(284486); DEDD(9191); |
| | | PTPN7(5778); TP53BP2(7159); FLJ40889(348654); NCOA1(8648); MAPRE3 |
| | | (22924); PPP1CB(5500); TCF7L1(83439); KCNIP3(30818); BRRN1(23397); |
| | | BCL2L11(10018); PROC(5624); RIF1(55183); TAIP-2(80034); ZAK |
| | | (51776); CFLAR(8837); C2orf17(79137); SEPT2(4735); E2F6(1876); RTN4 |
| | | (57142); EDAR(10913); ANAPC1(64682); IFIH1(64135); TNS1(7145); PAX3 |
| | | (5077); FANCD2(2177); VHL(7428); MLH1(4292); CTNNB1(1499); LIMD1 |
| | | (8994); RBM5(10181); RBM15B(29890); PCNP(57092); ACPP(55); SENP5 |
| | | (205564); RAF1(5894); ZNF445(353274); RASSF1(11186); FHIT(2272); |
| | | PIK3CB(5291); SIAH2(6478); LOC285382(285382); HD(3064); FGF5(2250); |
| | | SPP1(6696); NFKB1(4790); TLR2(7097); GAK(2580); APBB2(323); FLJ38991 |
| | | (285521); BTC(685); CCNI(10983); PRKG2(5593); KIAA1909(15 |

Table 4-146

3478); SKP2(6502); RAD17(5884); F2R(2149); APC(324); CSNK1G3(1456);
RAD50(10111); TCF7(6932); SLC36A1(206358); UNC5A(90249); DAP(1611);
PRLR(5618); MCC(4163); ATG12(9140); SEMA6A(57556); IRF1(3659);
NME5(8382); CD14(929); FGF1(2246); FBXW11(23291); DDX41(51428);
RIPK1(8737); LBRDC2(255488); E2F3(1871); LTA(4049); CSNK28(1460);
FOXP4(116113); TTBK1(84630); TNFAIP3(7128); HECA(51696); SASH1
(23328); NEDD9(4739); DHX16(8449); MDC1(9656); IER3(8870); DAXX
(1616); BAK1(578); TREML2(79865); CCND3(896); CUL7(9820); SUPT3H
(8464); SESN1(27244); PLAGL1(5325); AGPAT4(56895); PDCD2(5134);
SEPT7(989); EGFR(1956); RINT1(60561); BCAP29(55973); TRIAD3
(54476); PMS2(5395); CYCS(54205); CARD4(10392); SFRP4(6424); LOC401357
(401357); CDK6(1021); FASTK(10922); POLR3D(661); TNFRSF10C
(8794); PTK2B(2185); UNC5D(137970); TACC1(6867); INDOL1(169355);
TERF1(7013); WISP1(8840); C8orf55(51337); LZTS1(11178); TNFRSF10B
(8795); TNFRSF10D(8793); TNFRSF10A(8797); TRIM35(23087); CLU
(1191); KIF13B(23303); DKK4(27121); RAD21(5885); EXT1(2131); MTSS1
(9788); LYNX1(66004); C9orf47(286223); FREQ(23413); TAF1L(138474);
PTCH(5727); AKNA(80709); TNFSF15(9966); TRAF1(7185); FAM102A
(399665); CIZ1(25792); ZYG11BL(10444); UNC5B(219699); PTEN(5728);
FAS(355); LZTS2(84445); NOLC1(9221); SUFU(51684); PDCD11(22984);
TCF7L2(6934); LHPP(64077); DOCK1(1793); CUL2(8453); DLG5(9231);
SFRP5(6425); FGF8(2253); ACTR1A(10121); C10orf46(143384); MKI67
(4288); IFITM1(8519); STIM1(6786); CRY2(1408); F2(2147); DPF2(5977);
SSSCA1(10534); SIPA1(6494); PACS1(55690); FBXL11(22992); LRP5(4041);
CCND1(595); BIRC2(329); ATM(472); CD3E(916); CBL(867); FLJ45300
(399957); LOH11CR2A(4013); RHOG(391); APBB1(322); EIF4G2(1982);
TP53I11(9537); HRASLS3(11145); MAP3K11(4296); BRMS1(25855); MTL5
(9633); FGF4(2249); FGF3(2248); PAK1(5058); FZD4(8322); CASP4(837);
PPP2R1B(5519); BCL9L(283149); H2AFX(3014); LTBR(4055); WNT1
(7471); TEGT(7009); ESPL1(9700); PFDN5(5204); HOXC12(3228); PDE1B
(5153); RBMS2(5939); MDM2(4193); CRADD(8738); APAF1(317); KNTC1
(9735); NRIP2(83714); ING4(51147); LRP6(4040); BCAT1(586); KRAS
(3845); HDAC7A(51564); ARF3(377); FMNL3(91010); FAIM2(23017); B4GALNT1
(2583); CENTG1(116986); CDK4(1019); BTG1(694); HRK(8739);
DIABLO(56616); CDK2AP1(8099); HSMPP8(54737); TNFRSF19(55504);
RFP2(10206); ING1(3621); LATS2(26524); FOXO1A(2308); NEK3(4752);
BCL2L2(599); KIAA0247(9766); PSEN1(5663); VASH1(22846); TRAF3
(7187); KIAA0125(9834); ACIN1(22985); CIDEB(27141); GZMB(3002); LTBP2
(4053); NEK9(91754); CHES1(1112); PPP1R13B(23368); CCNB2(9133);
PML(5371); UBE3A(7337); AVEN(57099); SLC12A6(9990); EHD4(30844);
BNIP2(663); DAPK2(23604); CSNK1G1(53944); PDCD7(10081); RAB40C
(57799); CCNF(899); KREMEN2(79412); TNFRSF12A(51330); DNAJA3
(9093); PLK1(5347); CYLD(1540); GNAO1(2775); CCL22(6367); MMP15
(4324); ELMO3(79767); PARD6A(50855); CDK10(8558); AXIN1(8312);
RPUSD1(113000); PKMYT1(9088); GSPT1(2935); MAPK3(5595); SIAH1
(6477); FTS(64400); CSNK2A2(1459); SMPD3(55512); EXOSC6(118460);
FA2H(79152); HIC1(3090); GSG2(83903); ALOX12(239); TNFSF12
(8742); TRAF4(9618); NF1(4763); RAB11FIP4(84440); LIG3(3980); TADA2L
(6871); CNP(1267); FZD2(2535); MAPT(4137); NGFR(4804); CROP(51747);
PPM1D(8493); PRKCA(5578); SLC9A3R1(9368); CDK3(1018); SPHK1
(8877); BIRC5(332); CARD14(79092); GPS1(2873); MNT(4335); METT10D
(79066); P2RX1(5023); CAMTA2(23125); NALP1(22861); TP53(7157);
AURKB(9212); GAS7(8522); BECN1(8678); HDAC5(10014); MPO(4353);
ERN1(2081); UNC13D(201294); FLJ45079(400624); SOCS3(9021); ARHGDIA
(396); MAFG(4097); CETN1(1068); CABLES1(91768); DCC(1630);
MALT1(10892); PHLPP(23239); TCEB3C(162699); BCL2(596); FZR1(51343);
FEM1A(55527); DNM2(1785); SLC27A1(376497); UBA52(7311); KLHL26
(55295); NDUFA13(51079); KCTD15(79047); AXL(558); HNRPUL1
(11100); BCAM(4059); APOE(348); SPHK2(56848); BAX(581); BCL2L12
(83596); NALP2(55655); AURKC(6795); TNFSF7(970); PRDX2(7001); ELL
(8178); SIRT2(22933); AKT2(208); TGFB1(7040); ERF(2077); PPP1R13L
(10848); LIG1(3978); CARD8(22900); CGB7(94027); TRIB3(57761); PTPRA
(5786); CDC25B(994); CBFA2T2(9139); CEP250(11190); PPP1R16B(26051);
CSE1L(1434); SIRPB1(10326); RASSF2(9770); E2F1(1869); RBL1
(5933); RIMS4(140730); DIDO1(11083); SON(6651); S100B(6285); BCL2L

Table 4-147

| | | |
|---|---|---|
| | | 13(23786); NF2(4771); MCM5(4174); APOBEC3F(200316); EP300(2033); |
| | | MAPK8IP2(23542); MAPK1(5594); MN1(4330); TXN2(25828); SSTR3(6753); |
| | | CARD10(29775); NPTXR(23467); PDGFB(5155); CCNB3(85417); MLLT7 |
| | | (4303); TAF1(6872); BTK(695); SEPT6(23157); ARHGEF6(9459); FAM58A |
| | | (92002); |
| hsa-miR-651 | 2109; | PRAMEF9(343070); PRDM2(7799); SESN2(83667); MACF1(23499); PLK3 |
| | | (1263); CDC14A(8556); SLC30A7(148867); PEA15(8682); UHMK1(127933); |
| | | LAMC1(3915); PROX1(5629); C1orf69(200205); LOC440742(440742); |
| | | DFFA(1676); FUSIP1(10772); GNG12(55970); RBMXL1(494115); GBP3 |
| | | (2635); TMED5(50999); CTSS(1520); ASPM(259266); ZBTB41(360023); RAB3GAP2 |
| | | (25782); RAB10(10890); LYCAT(253558); BIRC6(57443); PRKCE |
| | | (5581); MSH2(4436); HTLF(3344); SLC4A10(57282); TAIP-2(80034); |
| | | KIAA1946(165215); PMS1(5378); FZD7(8324); BMPR2(659); COL4A3(1285); |
| | | OACT2(129642); WDR35(57539); EIF2AK2(5610); CCT4(10575); MAP3K2 |
| | | (10746); KIAA1715(80856); NCKAP1(10787); FZD5(7855); CUL3(8452); |
| | | ZNF662(389114); APPL(26060); SLC35A5(55032); ZDHHC23(254887); |
| | | SMC4L1(10051); MFN1(55669); SENP2(59343); OPA1(4976); OXTR |
| | | (5021); SRGAP3(9901); RAF1(5894); STAG1(10274); TFDP2(7029); TFRC |
| | | (7037); HD(3064); SLC4A4(8671); CCNG2(901); FGF5(2250); C4orf12(404201); |
| | | SYNPO2(171024); SRD5A2L2(253017); RASSF6(166824); LEF1(51176); |
| | | OTUD4(54726); ERBB2IP(55914); CCNB1(891); APC(324); RAD50 |
| | | (10111); ANKH(56172); PRLR(5618); HMGCS1(3157); PDE4D(5144); C5orf21 |
| | | (83989); MCC(4163); ATG12(9140); SEMA6A(57556); NME5(8382); NRG2 |
| | | (9542); CD74(972); TCF19(6941); AIF1(199); MAPK13(5603); ENPP4 |
| | | (22875); TTK(7272); AIG1(51390); NEDD9(4739); FKBP5(2289); SUPT3H |
| | | (8464); DST(667); ATG5(9474); SESN1(27244); LATS1(9113); MTRF1L |
| | | (54516); C6orf122(401288); PDCD2(5134); SEPT7(989); WBSCR19(285955); |
| | | LOC441257(441257); GNAI1(2770); MGC57359(441272); CNTNAP2 |
| | | (26047); TRIAD3(54476); SOSTDC1(25928); CDK6(1021); FZD3(7976); PI15 |
| | | (51050); EFCBP1(64168); PSD3(23362); TNFRSF10B(8795); TNFRSF10A |
| | | (8797); SFRP1(6422); UBE2W(55284); STK3(6788); ANKRD46(157567); |
| | | FBXO32(114907); ZNF250(58500); JAK2(3717); RECK(8434); FCMD |
| | | (2218); KLHL9(55958); GAS1(2619); PTCH(5727); CTNNAL1(8727); RBM18 |
| | | (92400); TSC1(7248); GPR158(57512); ZNF37A(7587); DKK1(22943); |
| | | SGPL1(8879); TNKS2(80351); ENTPD7(57089); BTRC(8945); C10orf111 |
| | | (221060); ITGB1(3688); PPP3CB(5532); SORCS1(114815); C10orf46(143384); |
| | | MKI67(4288); STIM1(6786); LOC387758(387758); LRRC55(219527); |
| | | DPF2(5977); PANX1(24145); BIRC3(330); SNF1LK9(23235); BLR1(643); |
| | | CBL(867); IGF2(3481); FBXO3(26273); SLC1A2(6506); HRASLS3(11145); |
| | | PAK1(5058); SESN3(143686); BTG4(54766); PPP2R1B(5519); EVA1(10205); |
| | | ATF7IP(55729); TEGT(7009); CDK2(1017); AICDA(57379); C12orf22 |
| | | (81566); CBX5(23468); CAPS2(84698); ANAPC7(51434); HSMPP8(54737); |
| | | RFP2(10206); ZIC5(85416); LIG4(3981); TXNDC(81542); PSEN1(5663); |
| | | CALM1(801); SIVA(10572); GZMH(2999); GZMB(3002); STRN3(29966); |
| | | MTAC2D1(128036); BUB1B(701); MAPK6(5597); LOC440295(440295); |
| | | RCCD1(91433); CSNK1G1(53944); PDCD7(10081); GSPT1(2935); BOLA2 |
| | | (552900); BIRC4BP(54739); MAP2K4(6416); NF1(4763); G6PC(2538); PRKCA |
| | | (5578); SPAG5(10615); TIAF1(9220); DDX52(11056); PTRF(284119); |
| | | BRCA1(672); ERN1(2081); ELAC1(55520); DOK6(220164); ROCK1(6093); |
| | | BCL2(596); UBA52(7311); AXL(558); ZNF480(147657); ZNF571(51276); |
| | | PLCB1(23236); SEC23B(10483); CTNNBL1(56259); CSE1L(1434); AURKA |
| | | (6790); SYCP2(10388); CABLES2(81928); TPTE(7179); EP300(2033); |
| | | PPARA(5465); CHEK2(11200); NHP2L1(4809); CXorf15(55787); PGK1 |
| | | (5230); DIAPH2(1730); BIRC4(331); TAB3(257397); DCX(1641); LAMP2(3920); |
| | | CUL4B(8450); ARHGEF6(9459); |
| hsa-miR-653 | 2110; | CDC42(998); SESN2(83667); RBBP4(5928); MACF1(23499); CDC20(991); |
| | | CDKN2C(1031); PRKACB(5567); HS2ST1(9653); BCL9(607); CKS1B(1163); |
| | | DAP3(7818); UHMK1(127933); FASLG(356); MDM4(4194); TGFB2(7042); |
| | | DISC1(27185); CCNL2(81669); MAD2L2(10459); FUSIP1(10772); CLSPN |
| | | (63967); HIVEP3(59269); BCL10(8915); EVI5(7813); NOTCH2(4853); |
| | | PRKAB2(5565); MCL1(4170); CD5L(922); C1orf198(84886); RHOB(388); |
| | | BIRC6(57448); PRKCE(5581); TCF7L1(83439); BRRN1(23397); PDCL3 |
| | | (79031); MAP4K4(9448); FLJ30294(130827); BCL2L11(10018); RIF1(55183); |
| | | ZAK(51776); CASP10(843); CASP8(841); CD28(940); COL4A3(1285); |
| | | RTN4(57142); GFPT1(2673); TIA1(7072); AFF3(3899); BUB1(699); IL1A |

Table 4-148

(3552); TLK1(9874); NCKAP1(10787); STAT1(6772); STK17B(9262); PGAP1
(80055); CLK1(1195); CUL3(8452); HDAC4(9759); VHL(7428); STT3B
(201595); PDCD6IP(10015); GNL3(26354); APPL(26060); PCNP(57092); SMC4L1
(10051); ECT2(1894); FXR1(8087); PSMD2(5708); SLC4A7(9497);
MAP4(4134); PB1(55193); DNASE1L3(1776); FHIT(2272); CNTN3(5067);
VGLL3(389136); KPNA1(3836); TFDP2(7029); XRN1(54464); SIAH2(6478);
TNFSF10(8743); HD(3064); CD38(952); EREG(2069); SEPT11(55752);
CCNG2(901); BMP2K(55589); FGF5(2250); NFKB1(4790); TLR2(7097);
STOX2(56977); AFAP(60312); PPBP(5473); CXCL9(4283); CXXC4(80319);
CASP6(839); ANKRD50(57182); SLC7A11(23657); ELF2(1998); FLJ32028
(201799); NEK1(4750); MFAP3L(9848); PDCD6(10016); SKP2(6502); NIPBL
(25836); FLJ13611(80006); RAD17(5884); F2R(2149); SCAMP1(9522);
CAMK4(814); APC(324); CSNK1G3(1456); RAD50(10111); PCDHGB4
(8641); G3BP(10146); GABRA1(2554); BNIP1(662); CNOT6(57472); PRLR
(5618); FLJ25680(134187); ATG12(9140); SAR1B(51128); ETF1(2107); FGF1
(2246); FBXW11(23291); RNF130(55819); RNF8(9025); BAG2(9532);
PTP4A1(7803); MYO6(4646); FOXO3A(2309); SART2(29940); TNFAIP3
(7128); SASH1(23328); NEDD9(4739); SUPT3H(8464); CLIC5(53405); DST
(667); COL9A1(1297); SYNCRIP(10492); CNR1(1268); ATG5(9474); SESN1
(27244); SGK(6446); PDCD2(5134); AHR(196); HDAC9(9734); SNX10
(29887); Ells1(222166); DKFZp61I2123(83637); POM121(9883); HBP1(26959);
BCAP29(55973); CASP2(835); CUL1(8454); TRIAD3(54476); SOSTDC1
(25928); CYCS(54205); ELMO1(9844); SFRP4(6424); CDK6(1021); TNKS
(8658); MTMR9(66036); BNIP3L(665); FZD3(7976); UNC5D(137970);
TACC1(6867); UBE2V2(7336); SULF1(23213); TERF1(7013); RDH10(157506);
E2F5(1875); RIPK2(8767); PTDSS1(9791); FZD6(8323); MYC(4609);
C8orf42(157695); TNFRSF10B(8795); TRIM35(23087); CLU(1191); SFRP1
(6422); NCOA2(10499); PAG1(55824); CCNE2(9134); TP53INP1(94241);
STK3(6788); TRPS1(7227); MTSS1(9788); CDC37L1(55664); JAK2(3717);
UNC13B(10497); RECK(8434); VPS13A(23230); NTRK2(4915); C9orf47
(286223); INVS(27130); ABL1(25); CDKN2B(1030); TSC1(7248); CARD9
(64170); ZMYND11(10771); C10orf9(219771); SIRT1(23411); PTEN(5728);
KIF11(3832); TCF7L2(6934); DOCK1(1793); C10orf111(221060); PARD3
(56288); ZWINT(11130); TMEM26(219623); DLG5(9231); SH3PXD2A
(9644); SMNDC1(10285); C10orf46(143384); BNIP3(664); TEAD1(7003);
HIPK3(10114); API5(8539); EXT2(2132); CCND1(595); BIRC3(330); BIRC2
(329); CUL5(8065); CD3G(917); MLL(4297); APBB1(322); FBXO3(26273);
SLC1A2(6506); PAK1(5058); FZD4(8322); JOSD3(79101); SESN3(143686);
PPP2R1B(5519); ZW10(9183); CCND2(894); BCL2L14(79370); CDKN1B
(1027); APOLD1(81575); ETNK1(55500); CDK2(1017); MDM2(4193);
APAF1(317); GAS2L3(283431); BRI3BP(140707); TMEM132B(114795);
RAD52(5893); LRP6(4040); BCAT1(586); YAF2(10138); LALBA(3906); DDIT3
(1649); WIF1(11197); PAWR(5074); PCTK2(5128); PPP1CC(5501); HRK
(8739); MPHOSPH9(10198); CHFR(55743); FGF9(2254); TNFRSF19
(55504); ATP8A2(51761); BRCA2(675); RFP2(10206); FLJ40296(122183);
MBNL2(10150); CUL4A(8451); TFDP1(7027); LATS2(26524); FOXO1A(2308);
LIG4(3981); ARHGAP5(394); ARF6(382); TXNDC(81542); TSHR(7253);
TDP1(55775); TRAF3(7187); NOVA1(4857); CDKL1(8814); DLG7(9787);
NEK9(91754); MOAP1(64112); BAG5(9529); BUB1B(701); FGF7(2252);
MAPK6(5597); LOC440295(440295); ABHD2(11057); IGF1R(3480); NDNL2
(56160); BMF(90427); PRTG(283659); RFXDC2(64864); BNIP2(663);
CSNK1G1(53944); TBC1D2B(23102); DNAJA3(9093); UBN1(29855);
CYLD(1540); RBL2(5934); CTCF(10664); NFAT5(10725); DNAJA2(10294);
SIAH1(6477); SMPD3(55512); TERF2(7014); HIC1(3090); PAFAH1B1
(5048); GSG2(83903); TNFSF12(8742); MAPK7(5598); NLK(51701); TRAF4
(9618); NF1(4763); UNC45B(146862); TADA2L(6871); CDC6(990); IGF2BP1
(10642); MSI2(124540); PPM1D(8493); TLK2(11011); PRKCA(5578);
BIRC5(332); BRCA1(672); MPO(4353); PTRH2(51651); C18orf54(162681);
MALT1(10892); PHLPP(23239); ROCK1(6093); ESCO1(114799); SS18
(6760); TXNL1(9352); BCL2(596); PARD6G(84552); FZR1(51343); CYP4F3
(4051); AXL(558); FOSB(2354); BCL2L12(83596); ZNF443(10224); DNASE2
(1777); PLCB1(23236); MAPRE1(22919); C20orf121(79183); STK4(6789);
PARD6B(84612); TXNDC13(56255); RBL1(5933); SYCP2(10388); DIDO1
(11083); SON(6651); CHAF1B(8208); PKNOX1(5316); DIP2A(23181);
SNF1LK(150094); ADORA2A(135); KREMEN1(83999); NF2(4771); E

Table 4-149

| | | |
|---|---|---|
| | | P300(2033); SEPT3(55984); BID(637); MAPK1(5594); CARD10(29775); DMC1 |
| | | (11144); TOB2(10766); SMC1L2(27127); PCTK1(5127); ZXDB(158586); |
| | | PGK1(5230); PABPC5(140886); BIRC4(331); RP6-213H19.1(51765); FIGF |
| | | (2277); RNF12(51132); ATRX(546); PCDH19(57526); ZMAT1(84460); |
| | | CUL4B(8450); |
| hsa-miR-654 | 2111; 2132; | PRKCZ(5590); TP73(7161); DFFB(1677); CLCN6(1185); PAX7(5081); EPHA8 |
| | | (2046); SFN(2810); SESN2(83667); KHDRBS1(10657); LCK(3932); |
| | | MACF1(23499); NFYC(4802); CDC20(991); CD2(914); BCL9(607); FDPS |
| | | (2224); PEA15(8682); IL19(29949); C1orf95(375057); CIAS1(114548); TNFRSF14 |
| | | (8764); MELL1(79258); TNFRSF9(3604); ZBTB17(7709); KIF17(57576); |
| | | HSPG2(3339); YARS(8565); BCL10(8915); GFI1(2672); NOTCH2(4853); |
| | | MCL1(4170); PYGO2(90780); KIF21B(23046); GREB1(9687); RHOB |
| | | (388); BIRC6(57448); PRKCE(5581); HK2(3099); TCF7L1(83439); KCNIP3 |
| | | (30818); PROC(5624); CFLAR(8837); CASP10(843); CD28(940); ALS2CR19 |
| | | (117583); COL4A3(1285); PTMA(5757); GPC1(2817); BOK(666); CGREF1 |
| | | (10669); PPM1G(5496); RTN4(57142); EDAR(10913); ANAPC1(64682); |
| | | PAX8(7849); BIN1(274); IFIH1(64135); FZD5(7855); PAX3(5077); HDAC4 |
| | | (9759); VHL(7428); MLH1(4292); APPL(26060); KLHDC6(166348); RNF7 |
| | | (9616); CHRD(8646); TADA3L(10474); AXUD1(64651); TMEM16K(55129); |
| | | MGLL(11343); SH3BP2(6452); HD(3064); FLJ35725(152992); FGF5 |
| | | (2250); NFKB1(4790); GAK(2580); SLC26A1(10861); LETM1(3954); SFRP2 |
| | | (6423); CARD6(84674); ERBB2IP(55914); APC(324); KIAA0141(9812); |
| | | UNC5A(90249); PRKAA1(5562); UNG2(10309); ATG12(9140); IRF1(3659); |
| | | CDC23(8697); FCHSD1(89848); CAMK2A(815); FBXW11(23291); PDLIM7 |
| | | (9260); RIPK1(8737); AIF1(199); CSNK2B(1460); MAPK13(5603); |
| | | PARC(23113); TNFAIP3(7128); SASH1(23328); IER3(8870); DAXX(1616); |
| | | IHPK3(117283); CUL7(9820); TNFRSF21(27242); DST(667); KATNA1(11104); |
| | | LATS1(9113); AHR(196); HDAC9(9734); EGFR(1956); FZD9(8326); |
| | | STAG3(10734); TRIAD3(54476); CARD4(10392); ELMO1(9844); INHBA |
| | | (3624); YWHAG(7532); PTN(5764); HIPK2(28996); CDK5(1020); FASTK(10922); |
| | | TNFRSF10C(8794); PTK2B(2185); UNC5D(137970); SULF1(23213); |
| | | RIPK2(8767); MYC(4609); C8orf55(51337); LZTS1(11178); BIN3(55909); |
| | | TNFRSF10B(8795); TNFRSF10D(8793); TRIM35(23087); RAD21(5885); |
| | | MTSS1(9788); CDK9(1025); FREQ(23413); ABL1(25); GRIN1(2902); FLJ20245 |
| | | (54863); TAF1L(138474); BAG1(573); FANCG(2189); SEMA4D |
| | | (10507); PTCH(5727); TNFSF15(9966); TRAF1(7185); CIZ1(25792); TSC1 |
| | | (7248); ANAPC2(29882); UNC5B(219699); FRAT1(10023); LZTS2(84445); |
| | | TCF7L2(6934); DOCK1(1793); PAPD1(55149); CUL2(8453); ZWINT(11130); |
| | | C10orf46(143384); HIPK3(10114); MDK(4192); C11orf11(747); SSSCA1 |
| | | (10534); SIPA1(6494); RAB1B(81876); PELI3(246330); LRP5(4041); CCND1 |
| | | (595); FADD(8772); CUL5(8065); APBB1(322); DKK3(27122); TP53I11 |
| | | (9537); MAP3K11(4296); RELA(5970); CLCF1(23529); PPP1CA(5499); |
| | | FGF19(9965); FGF4(2249); FGF3(2248); CASP4(837); CASP5(838); CASP1 |
| | | (834); BCL9L(283149); PVRL1(5818); TSPAN9(10867); CCND2(894); TNFRSF7 |
| | | (939); ESPL1(9700); MDM2(4193); APAF1(317); FED10(11211); |
| | | LRP6(4040); HDAC7A(51564); MCRS1(10445); FAIM2(23017); C12orf22 |
| | | (81566); WIF1(11197); NT5DC3(51559); MSI1(4440); SCARB1(949); ING1 |
| | | (3621); TFDP1(7027); LATS2(26524); KIAA0247(9766); VASH1(22846); TRAF3 |
| | | (7187); KIAA0284(283638); CIDEB(27141); LTBP2(4053); CHES1 |
| | | (1112); PPP1R13B(23368); AKT1(207); JAG2(3714); FGF7(2252); MAPK6 |
| | | (5597); TLN2(83660); ZN7609(23060); CSK(1445); ALPK3(57538); NDN(4692); |
| | | SLC12A6(9990); PDCD7(10081); DKFZp434I1020(196968); RAB40C |
| | | (57799); TSC2(7249); TRAF7(84231); KREMEN2(79412); DNASE1(1773); |
| | | PLK1(5347); RBL2(5934); NOL3(8996); CTCF(10664); LRRC50(123872); |
| | | FLJ45121(400556); CDT1(81620); C16orf55(124045); MGC24381(115939); |
| | | ADCY9(115); FAM100A(124402); ERN2(10595); MAPK3(5595); SEPT1 |
| | | (1731); PYCARD(29108); CSNK2A2(1459); SLC12A4(6560); SMPD3(55512); |
| | | TERF2(7014); CBFA2T3(863); PCOLN3(5119); TNFSF12(8742); ALOX15B |
| | | (247); MAPK7(5598); NLK(51701); NF1(4763); LASP1(3927); CDC6 |
| | | (990); MAPT(4137); NGFR(4804); NXPH3(11248); PRKCA(5578); SLC9A3R1 |
| | | (9368); CDK3(1018); SPHK1(8877); CBX2(84733); CARD14(79092); |
| | | raptor(57521); BAIAP2(10458); ANAPC11(51529); SLC43A2(124935); |
| | | MNT(4335); P2RX1(5023); AURKB(9212); GAS7(8522); GAS2L2(246176); |
| | | BRCA1(672); HDAC5(10014); C1QL1(10882); PLCD3(113026); PLEKHM1 |
| | | (9842); PHB(5245); ERN1(2081); SOCS3(9021); TBC1D16(125058); M |

Table 4-150

| | | |
|---|---|---|
| | | APRE2(10982); MAPK4(5596); DCC(1630); PHLPP(23239); BCL2(596); TXNL4A |
| | | (10907); CDC34(997); CSNK1G2(1455); FZR1(51343); TNFSF9(8744); |
| | | MAP2K7(5609); DNM2(1785); F2RL3(9002); PIK3R2(5296); APLP1 |
| | | (333); MAP3K10(4294); AXL(558); ARHGEF1(9138); APOE(348); FOSB(2354); |
| | | SPHK2(56848); BAX(581); ATF5(22809); DIRAS1(148252); AES(166); |
| | | PIP5K1C(23396); CDC37(11140); PRDX2(7001); PRX(57716); TGFB1 |
| | | (7040); ERF(2077); ERCC2(2068); PPP1R13L(10848); BBC3(27113); LIG1 |
| | | (3978); TRIB3(57761); CDC25B(994); ZGPAT(84619); BCL2L1(598); PLAGL2 |
| | | (5326); ZHX3(23051); HRH3(11255); CABLES2(81928); DIDO1(11083); |
| | | SON(8851); CHAF1B(8208); MX1(4599); COL6A2(1292); SNF1LK(150094); |
| | | SEPT5(5413); MIF(4282); KREMEN1(83999); GAS2L1(10634); NF2 |
| | | (4771); MCM5(4174); EP300(2033); SEPT3(55964); GTSE1(51512); MAPK8IP2 |
| | | (23542); MAPK1(5594); PPM1F(9647); MN1(4330); LIF(3976); KIAA1904 |
| | | (114794); PDGFB(5155); TOB2(10766); NHP2L1(4809); SMC1L2(27127); |
| | | HDAC6(10013); TAF1(6872); CD40LG(959); CXorf36(79742); SEPT6 |
| | | (23157); CUL4B(8450); PDCD8(9131); HCFC1(3054); MECP2(4204); |
| hsa-miR-657 | 2112; | SFN(2810); SESN2(83667); EPB41(2035); HDAC1(3065); MACF1(23499); |
| | | KIAA0467(23334); CDKN2C(1031); PROK1(84432); FAM46C(54855); BCL9 |
| | | (607); BNIPL(149428); PEA15(8682); FASLG(356); SRGAP2(23380); |
| | | PFKFB2(5208); TRAF5(7188); CIAS1(114548); CDC2L2(985); DFFA(1676); |
| | | ZBTB17(7709); E2F2(1870); YARS(8565); TESK2(10420); PIK3R3(8503); |
| | | FAF1(11124); SASS6(163786); NOTCH2(4853); CD5L(922); KIAA0040 |
| | | (9674); ASTN(460); NUCKS1(64710); IL10(3586); SLC30A1(7779); ITPKB |
| | | (3707); PDCL3(79031); BCL2L11(10018); TAIP-2(80034); CASP10(843); |
| | | CASP8(841); FZD7(8324); COL4A3(1285); FLJ45964(401040); YWHAQ |
| | | (10971); PPM1G(5496); PRKCD3(23683); TGFA(7039); BIN1(274); GPR155 |
| | | (151556); STAT1(6772); STK17B(9262); ZNF142(7701); HDAC4(9759); FANCD2 |
| | | (2177); PDCD6IP(10015); MLH1(4292); DLEC1(9940); CTNNB1(1499); |
| | | TDGF1(6997); APPL(26060); ATXN7(6314); EAF2(55840); NEK11(79858); |
| | | RNF7(9616); TRAIP(10293); TUSC4(10641); PB1(55193); MAGI1 |
| | | (9223); RAB43(339122); ETV5(2119); DKFZP564O0823(25849); CCNG2(901); |
| | | FGF2(2247); PLK4(10733); GABRA4(2557); UGT2B15(7366); UNC5C |
| | | (8633); ADH4(127); DKK2(27123); LOC152485(152485); SFRP2(6423); |
| | | VEGFC(7424); NIPBL(25836); MRPS30(10884); UNC5A(90249); N4BP3 |
| | | (23138); SQSTM1(8878); TPPP(11076); DAP(1611); PRLR(5618); MEF2C |
| | | (4208); MCC(4163); ATG12(9140); SEMA6A(57556); CDC25C(995); CAMK2A |
| | | (815); CD74(972); KIAA0676(23061); RNF130(55819); TCF19(6941); LTA |
| | | (4049); AIF1(199); MAPK13(5603); PARC(23113); HSP90AB1(3326); TTK |
| | | (7272); FOXO3A(2309); HECA(51696); SASH1(23328); NEDD9(4739); |
| | | CUL7(9820); DST(667); B3GAT2(135152); SESN1(27244); PLAGL1(5325); |
| | | LATS1(9113); HDAC9(9734); KIAA1706(80820); EGFR(1956); FZD9(8326); |
| | | FZD1(8321); CASP2(835); CUL1(8454); TRIAD3(54476); PMS2(5395); |
| | | CARD4(10392); SFRP4(6424); INHBA(3624); PURB(5814); CALN1(83698); |
| | | CDK5(1020); TNKS(8658); SLC25A37(51312); PTK2B(2185); ESCO2 |
| | | (157570); UNC5D(137970); TACC1(6867); TERF1(7013); RIPK2(8767); PTDSS1 |
| | | (9791); FZD6(8323); LZTS1(11178); ENTPD4(9583); DUSP4(1846); |
| | | FGFR1(2260); SFRP1(6422); FBXO32(114907); FAM84B(157638); UNC13B |
| | | (10497); FLJ45537(401535); DAPK1(1612); INVS(27130); FCMD(2218); |
| | | DOLPP1(57171); ABL1(25); TRAF1(7185); CIZ1(25792); TSC1(7248); |
| | | C10orf9(219771); DKK1(22943); UNC5B(219699); FAS(355); SUFU(51684); |
| | | CSPG6(9126); TCF7L2(6934); LOC387646(387646); PARD3(56288); FZD8 |
| | | (8325); ZNF25(219749); ZWINT(11130); KCNMA1(3778); DLG5(9231); |
| | | SFRP5(6425); SORCS1(114815); SMNDC1(10285); MKI67(4288); TEAD1 |
| | | (7003); CRY2(1408); F2(2147); LRP5(4041); BIRC2(329); CUL5(8065); |
| | | ATM(472); CDKN1C(1028); RHOG(391); APBB1(322); DKK3(27122); WT1 |
| | | (7490); CLCF1(23529); SHANK2(22941); BCL9L(283149); C11orf45(219833); |
| | | CCND2(894); LTBR(4055); CNAP1(9918); PTPN6(5777); SPATS2(65244); |
| | | RBMS2(5939); SOCS2(8835); APAF1(317); PTPN11(5781); LRP6(4040); |
| | | C12orf22(81566); SMUG1(23583); CIP29(84324); WIF1(11197); DUSP6 |
| | | (1848); NT5DC3(51559); FGF9(2254); TNFRSF19(55504); CDK8(1024); |
| | | BRCA2(675); RFP2(10206); CUL4A(8451); NEK3(4752); CDKN3(1033); |
| | | MNAT1(4331); PSEN1(5663); DLK1(8788); TRAF3(7187); ACIN1(22985); |
| | | STRN3(29966); NFKBIA(4792); ATP6V1D(51382); LTBP2(4053); CHES1 |
| | | (1112); BAG5(9529); PPP1R13B(23368); CRI1(23741); ZNF609(23060); |
| | | ARNT2(9915); AKAP13(11214); COPS2(9318); BNIP2(663); CSNK1G1(5 |

Table 4-151

| | | |
|---|---|---|
| | | 3944); C15orf38(348110); TSC2(7249); TRAF7(84231); CARD15(64127); CYLD |
| | | (1540); RBL2(5934); KATNB1(10300); E2F4(1874); AXIN1(8312); XYLT1 |
| | | (64131); MAPK3(5595); SEPT1(1731); FTS(64400); CIAPIN1(57019); |
| | | SMPD3(55512); MAF(4094); PCOLN3(5119); HIC1(3090); FGF11(2256); |
| | | ALOX15B(247); NTN1(9423); NF1(4763); RAB11FIP4(84440); LIG3(3980); |
| | | WIRE(147179); FZD2(2535); MAPT(4137); PRKCA(5578); SLC9A3R1 |
| | | (9368); CDK3(1018); SPHK1(8877); GPS1(2873); MNT(4335); CAMTA2(23125); |
| | | SPAG5(10615); TIAF1(9220); ACCN1(40); PLXDC1(57125); BRCA1 |
| | | (672); MPO(4353); SOCS3(9021); CBX4(8535); MAPRE2(10982); MAPK4 |
| | | (5596); DCC(1630); C18orf26(284254); ST8SIA3(51046); TNFRSF11A(8792); |
| | | PHLPP(23239); SERPINB2(5055); NFATC1(4772); ROCK1(6093); BCL2 |
| | | (596); ZADH2(284273); FZR1(51343); KLF2(10365); UBA52(7311); APLP1 |
| | | (333); LGALS7(3963); MAP3K10(4294); ARHGEF1(9138); BCL3(602); |
| | | APOE(348); SPHK2(56848); NALP2(55655); AURKC(6795); MATK(4145); |
| | | PRDX2(7001); DNASE2(1777); ZNF529(57711); DEDD2(162989); VISA |
| | | (57506); PLCB1(23236); MAPRE1(22919); PARD6B(84612); PCNA(5111); |
| | | PLAGL2(5326); PTPRT(11122); KCNS1(3787); CTCFL(140690); CABLES2 |
| | | (81928); DIDO1(11083); SON(6651); C21orf125(284836); APP(351); C21orf25 |
| | | (25966); SNF1LK(150094); ICOSLG(23308); ADORA2A(135); MCM5(4174); |
| | | PGEA1(25776); SEPT3(55964); GTSE1(51512); MAPK1(5594); |
| | | CARD10(29775); PDGFB(5155); CHKB(1120); WWC3(55841); PCTK1(5127); |
| | | EDA(1896); ZCCHC16(340595); ARHGAP6(395); FIGF(2277); PCYT1B |
| | | (9468); SMC1L1(8243); MBNL3(55796); |
| hsa-miR-661 | 2113; | SAMD11(148398); TP73(7161); DFFB(1677); KCNAB2(8514); H6PD(9563); |
| | | TNFRSF1B(7133); PAX7(5081); SFN(2810); NUDC(10726); SESN2(83667); |
| | | RBBP4(5928); MACF1(23499); NFYC(4802); CDC20(991); CDC7(8317); |
| | | CDC14A(8556); PROK1(84432); RAP1A(5906); CD2(914); BCL9(607); |
| | | DAP3(7818); UHMK1(127933); PPP1R12B(4660); ADORA1(134); PRELP |
| | | (5549); MDM4(4194); TRAF5(7188); C1orf95(375057); C1orf69(200205); |
| | | TNFRSF18(8784); TNFRSF14(8764); CHD5(26038); PLEKHG5(57449); |
| | | TNFRSF9(3604); FRAP1(2475); MTHFR(4524); CASP9(842); ZBTB17(7709); |
| | | RCC2(55920); E2F2(1870); RIMS3(9783); TESK2(10420); BCL10(8915); |
| | | SORT1(6272); NOTCH2(4853); PYGO2(90780); DEDD(9191); IL10(3586); |
| | | GREB1(9687); RHOB(388); BIRC6(57448); PRKCE(5581); HK2(3099); |
| | | KCNIP3(30818); BRRN1(23397); ARHGEF4(50649); TAIP-2(80034); CFLAR |
| | | (8837); CASP10(843); CASP8(841); FZD7(8324); COL4A3(1285); RNPEPL1 |
| | | (57140); BOK(666); CGREF1(10669); FLJ34931(388939); CARD12 |
| | | (58484); EIF2AK2(5610); RTN4(57142); EDAR(10913); BUB1(699); ANAPC1 |
| | | (64682); BIN1(274); MCM6(4175); STAT1(6772); FZD5(7855); FLJ43879 |
| | | (401039); HDAC4(9759); KIF1A(547); FANCD2(2177); VHL(7428); |
| | | MLH1(4292); DLEC1(9940); TDGF1(6997); GNL3(26354); APPL(26060); |
| | | ARGFX(503582); FAIM(55179); RNF7(9616); TP73L(8626); TADA3L(10474); |
| | | AXUD1(64651); TUSC4(10641); NEK4(6787); FHIT(2272); UBE1C(9039); |
| | | KIAA2018(205717); ATR(545); MASP1(5648); HD(3064); FLJ35725 |
| | | (152992); SEPT11(55752); CCNG2(901); FGF2(2247); PLK4(10733); CCNI |
| | | (10983); CASP3(836); CARD6(84674); MRPS30(10884); APC(324); RAD50 |
| | | (10111); TCF7(6932); WDR55(54853); PCDEB9(56127); PCDHB13(56123); |
| | | NDST1(3340); CPLX2(10814); UNC5A(96249); PRLR(5618); PRKAA1 |
| | | (5562); UNG2(10309); MCC(4163); ATG12(9140); SEMA6A(57556); CAMK2A |
| | | (815); CD74(972); RIPK1(8737); TNF(7124); CSNK2B(1460); MAPK13 |
| | | (5603); PARC(23113); VEGF(7422); PTP4A1(7803); FOXO3A(2309); |
| | | HECA(51696); SASH1(23328); NEDD9(4739); MDC1(9656); BAK1(578); IHPK3 |
| | | (117283); CUL7(9820); TNFRSF21(27242); DST(667); LATS1(9113); |
| | | PDCD2(5134); FOXK1(221937); AHR(196); SEPT7(989); STK17A(9263); |
| | | ADCY1(107); FLJ10099(55069); FZD1(8321); STAG3(10734); HBP1(26959); |
| | | BCAP29(55973); CASP2(835); CUL1(8454); MAD1L1(8379); TRIAD3 |
| | | (54476); PMS2(5395); CYCS(54205); ELMO1(9844); TMED4(222068); IGFBP3 |
| | | (3486); LOC401357(401357); CDK6(1021); ZC3HC1(51530); HIPK2 |
| | | (28996); KIAA0773(9715); POLR3D(661); PDLIM2(64236); PTK2B(2185); |
| | | ESCO2(157570); BAG4(9530); SULF1(23213); TERF1(7013); RIPK2(8767); |
| | | EBAG9(9166); MAPK15(225689); C8orf30A(51236); LRRC14(9684); LZTS1 |
| | | (11178); TNFRSF10B(8795); TNFRSF10A(8797); CLU(1191); SFRP1 |
| | | (6422); EXT1(2131); MTSS1(9788); ANKRD15(23189); CDC37L1(55664); |
| | | UNC13B(10497); DAPK1(1612); INVS(27130); PPP2R4(5524); FREQ(23413); |
| | | ABL1(25); LCN12(286256); PTGDS(5730); FLJ20245(54863); BAG |

Table 4-152

1(573); FANCG(2189); SEMA4D(10507); PTCH(5727); TRIM14(9830); TRAF1
(7185); CIZ1(25792); RAPGEF1(2889); TSC1(7248); CARD9(64170);
C9orf140(89958); ANAPC2(29882); DKK1(22943); SIRT1(23411); SGPL1
(8879); UNC5B(219699); FAS(355); LZTS2(84445); BTRC(8945); ADARB2
(105); IL2RA(3559); PAPD1(55149); CUL2(8453); ZWINT(11130); DLG5
(9231); SFRP5(6425); FBXW4(6468); SH3PXD2A(9644); SMNDC1(10285);
C10orf46(143384); MKI67(4288); IFITM1(8519); STIM1(6786); WEE1(7465);
MDK(4192); DAK(26007); INCENP(3619); LGALS12(85329); VEGFB
(7423); DPF2(5977); SSSCA1(10534); SIPA1(6494); RPS6KB2(6199); BIRC3
(330); ATM(472); LOH11CR2A(4013); HRAS(3265); IGF2(3481); APBB1
(322); DKK3(27122); WT1(7490); ABTB2(25841); BAD(572); CDCA5(113130);
MAP3K11(4296); RELA(5970); PPP1CA(5499); FGF4(2249); LRRC32
(2615); FZD4(8322); EXPH5(23086); BCL9L(283149); H2AFX(3014);
CCND2(894); LTBR(4055); CNAP1(9918); PTPN6(5777); BCL2L14(79370);
GPD1(2819); ESPL1(9700); CDK2(1017); RBMS2(5939); MDM2(4193);
APAF1(317); NOL1(4839); BCAT1(586); KRAS(3845); HDAC7A(51564);
MCRS1(10445); FAIM2(23017); C12orf22(81566); WIF1(11197); C12orf43
(64897); CDK2AP1(8099); SCARB1(949); CHFR(55743); HSMPP8(54737);
BRCA2(675); RB1(5925); RFP2(10206); SUGT1(10910); FOXO1A(2308);
NEK3(4752); BCL2L2(599); KIAA0323(23351); PSEN1(5663); VASH1(22846);
TRAF3(7187); KNS2(3831); SIVA(10572); KIAA0125(9834); ACIN1
(22985); CIDEB(27141); LTBP2(4053); NEK9(91754); CHES1(1112); MOAP1
(64112); BAG5(9529); PPP1R13B(23368); AKT1(207); JAG2(3714); KLF13
(51621); BUB1B(701); THSD4(79875); PML(5371); CSK(1445); IL16
(3603); FLJ22795(80154); SCAND2(54581); NDN(4692); BMF(90427); FLJ43339
(388115); CSNK1G1(53944); PDCD7(10081); C15orf38(343110); TSC2
(7249); CCNF(899); KREMEN2(79412); DNAJA3(9093); PLK1(5347);
SPN(6693); CARD15(64127); CYLD(1540); RBL2(5934); GNAO1(2775);
NOL3(8996); E2F4(1874); ELMO3(79767); CDT1(81620); AXIN1(8312); PKD1
(5310); PKMYT1(9088); GSPT1(2935); GGA2(23062); ERN2(10595);
P8(26471); MAPK3(5595); SEPT1(1731); STX1B2(112755); CIAPIN1(57019);
CMTM4(146223); TRADD(8717); CTRL(1506); SLC12A4(6560); SMPD3
(55512); TERF2(7014); TXNL4B(54957); HIC1(3090); NLGN2(57555);
TNFSF12(8742); ALOX15B(247); MAPK7(5598); SARM1(23098); NF1(4763);
LIG3(3980); TADA2L(6871); MLLT6(4302); STARD3(10948); CDC6
(990); FZD2(2535); MAPT(4137); GOSR2(9570); PNPO(55163); NGFR(4804);
CROP(51747); MAP3K3(4215); PRKCA(5578); KPNA2(3838); RAB37
(326624); SLC9A3R1(9368); CDK3(1018); SPHK1(8877); SEPT9(10801); CARD14
(79092); GPS1(2873); MNT(4335); METT10D(79066); P2RX1(5023);
NALP1(22861); GPS2(2874); TP53(7157); GAS7(8522); TOM1L2(146691);
SPAG5(10615); GAS2L2(246176); HAP1(9001); BECN1(8678); HDAC5
(10014); GJA7(10052); PLCD3(113026); SEPT4(5414); CYB561(1534); CASKIN2
(57513); MXRA7(439921); TMC6(11322); SOCS3(9021); CBX4(8535);
TMEM105(284186); PCYT2(5833); CSNK1D(1453); EMILIN2(84034);
CIDEA(1149); MAPK4(5596); MALT1(10892); PHLPP(23239); SERPINB2
(5055); SCAMP4(113178); CSNK1G2(1455); GADD45B(4616); FZR1
(51343); GIPC3(126326); TNFSF9(8744); PIN1(5300); DNM2(1785); PIK3R2
(5296); UBA52(7311); LGALS7(3963); IXL(55588); MAP3K10(4294); AXL
(558); MGC20255(90324); ARHGEF1(9138); PVR(5817); APOE(348);
HIF3A(64344); GRLF1(2909); SPHK2(56848); RPS11(6205); PPP2R1A(5518);
RPL28(6158); FLJ45684(400666); GNG7(2788); AES(166); MATK(4145);
TNFSF7(970); TNFSF14(8740); ELAVL1(1994); RAB3D(9545); ELAVL3
(1995); DNASE2(1777); LASS1(10715); SIRT2(22933); DEDD2(162989);
PPP1R13L(10848); DMWD(1762); BBC3(27113); LIG1(3978); CARD8
(22900); KLK10(5655); A1BG(1); PLCB1(23236); CEP250(11190); KCNK15
(60598); STK4(6789); UBE2C(11065); PARD6B(84612); CSNK2A1(1457);
AURKA(6790); CABLES2(81928); DIDO1(11083); GM632(57473); SON
(6651); C21orf125(284836); PDXK(8566); AGPAT3(56894); SNF1LK(150094);
C21orf58(54058); SEPT5(5413); SMARCB1(6598); ADORA2A(135);
GAS2L1(10634); NF2(4771); MCM5(4174); APOL6(80830); SYNGR1(9145);
EP300(2033); ZC3H7B(23264); SEPT3(55964); PPARA(5465); GTSE1
(51512); ADM2(79924); BID(637); FLJ21125(79680); MAPK1(5594); PPM1F
(9647); MN1(4330); CBX6(23466); TOB2(10766); SMC1L2(27127); PCTK1
(5127); HDAC6(10013); MLLT7(4303); BIRC4(331); IKBKG(8517); PPP2R3B
(28227); SMC1L1(8243); SEPT6(23157); CUL4B(8450); FAM58A(

Table 4-153

| | | |
|---|---|---|
| | | 92002); HCFC1(3054); |
| hsa-miR-769-5p | 2114; | SAMD11(148398); ZBTB40(9923); CCDC21(64793); PTPRU(10076); MACF1 |
| | | (23499); C1orf84(149469); PLK3(1263); C1orf83(127428); SH3GLB1 |
| | | (51100); SLC35A3(23443); ANXA9(8416); NAV1(89796); PRELP(5549); LYPLAL1 |
| | | (127018); MELL1(79258); CTNNBIP1(56998); DFFA(1676); RCC2 |
| | | (55920); E2F2(1870); ST7L(54879); NOTCH2(4853); ASPM(259266); IL10 |
| | | (3586); TP53BP2(7159); LYST(1130); AKT3(10000); LPIN1(23175); LBH |
| | | (81606); BIRC6(57448); HTRA2(27429); HK2(3099); CFLAR(8837); NRP2 |
| | | (8828); COL4A3(1285); PSMD1(5707); ADCY3(109); RTN4(57142); STAT1 |
| | | (6772); CUL3(8452); HDAC4(9759); CTNNB1(1499); DAG1(1605); APPL |
| | | (26060); NUDT16(131870); CLDN18(51208); KNG1(3827); CIDEC(63924); |
| | | ZNF445(353274); VGLL3(389136); MUC4(4585); WHSC1(7468); HD(3064); |
| | | SOD3(6649); SLC34A2(10568); EREG(2069); SEPT11(55752); SPATA4 |
| | | (132851); KIAA1909(153478); PDCD6(10016); SKP2(6502); R7BP(401190); |
| | | GRAMD3(65983); TCF7(6932); PSD2(84249); PRO1580(55374); PRKAA1 |
| | | (5562); MCC(4163); SEMA6A(57556); SAR1B(51128); NME5(8382); |
| | | CDC25C(995); LRRTM2(26045); FBXW11(23291); GRM6(2916); AIF1(199); |
| | | PARC(23113); SASH1(23328); ZDHHC14(79683); IER3(8870); BAK1(578); |
| | | IHPK3(117283); MDGA1(266727); DST(667); SGK(6446); BCLAF1(9774); |
| | | PLAGL1(5325); EPM2A(7957); EGFR(1956); FZD9(8326); FZD1(8321); |
| | | TRRAP(8295); HBP1(26959); BCAP29(55973); RBM33(155435); CALN1 |
| | | (83698); YWHAG(7532); CDK6(1021); IMPDH1(3614); TNFRSF10C(8794); |
| | | SCARA3(51435); UNC5D(137970); ATP6V1C1(528); RHPN1(114822); |
| | | ZNF623(9831); BIN3(55909); TNFRSF10B(8795); TNFRSF10D(8793); |
| | | ZNF395(55893); SFRP1(6422); STK3(6788); EXT1(2131); CPSF1(29894); |
| | | RECK(8434); NTRK2(4915); DAPK1(1612); C9orf47(286223); SYK(6850); |
| | | SET(6418); COL5A1(1289); CDKN2B(1030); SEMA4D(10507); PTCH(5727); |
| | | TNFSF8(944); ASB6(140459); ABO(28); CDC2(983); UNC5B(219699); |
| | | SFTPA1(6435); HIF1AN(55662); BTRC(8945); NOLC1(9221); SUFU(51684); |
| | | CSPG6(9126); C10orf10(11067); MARCH8(220972); ZWINT(11130); |
| | | KCNMA1(3778); SFTPA2(6436); HTR7(3363); FAM26C(255022); COL17A1 |
| | | (1308); VAX1(11023); IFITM1(8519); INCENP(3619); SIPA1(6494); ADRBK1 |
| | | (156); BIRC3(330); CUL5(8065); ATM(472); TMPRSS4(56649); CD3E |
| | | (916); RELA(5970); SHANK2(22941); PGM2L1(283209); PPP2R1B(5519); |
| | | BCL9L(283149); PTPN6(5777); BCL2L14(79370); FGD4(121512); DIP2B |
| | | (57609); TCF1(6927); FZD10(11211); FGF6(2251); LRP6(4040); BCAT1 |
| | | (586); MCRS1(10445); FMNL3(91010); DDIT3(1649); PCTK2(5128); NT5DC3 |
| | | (51559); BRCA2(675); ING1(3621); KLF12(11278); KCTD12(115207); |
| | | BCL2L2(599); PSEN1(5663); COQ6(51004); GZMH(2999); C14orf43 |
| | | (91748); PGF(5228); NEK9(91754); SPTLC2(9517); CHES1(1112); PML(5371); |
| | | IGF1R(3480); LRRK1(79705); SLC12A6(9990); CSNK1G1(53944); FLJ22795 |
| | | (80154); TBL3(10607); KREMEN2(79412); KIAA0350(23274); E2F4 |
| | | (1874); ELMO3(79767); TFAP4(7023); ERN2(10595); SLC12A4(6560); |
| | | SMPD3(55512); PCOLN3(5119); LIG3(3980); EME1(146956); CROP(51747); |
| | | PRKCA(5578); AMZ2(51321); AURKB(9212); SLC25A35(399512); FAM106A |
| | | (80039); FLJ31952(146857); GAS2L2(246176); ERN1(2081); HELZ |
| | | (9931); TBC1D16(125058); PDE6G(5148); MAFG(4097); C17orf62(79415); |
| | | CABLES1(91768); ZADH2(284273); FZR1(51343); LOC388503(388503); |
| | | FLJ45910(388512); APOE(348); FOSB(2354); SPHK2(56848); PPP2R1A |
| | | (5518); LOC390980(390980); ZNF549(256051); TNFSF14(8740); ZNF561 |
| | | (93134); PRX(57716); PPP1R13L(10848); SLC8A2(6543); LIG1(3978); |
| | | KLK10(5655); ZNF665(79788); CDS2(8760); PPP1R16B(26051); PARD6B |
| | | (84612); PCNA(5111); DIDO1(11083); ADARB1(104); SNF1LK(150094); |
| | | S100B(6285); BCL2L13(23786); GAS2L1(10634); EP300(2033); MAPK8IP2 |
| | | (23542); DGCR14(8220); MAPK1(5594); PPM1F(9647); CARD10(29775); |
| | | PDGFB(5155); RP11-321G1.1(54885); IKBKG(8517); TFE3(7030); RGAG4 |
| | | (340526); RP11-130N24.1(340533); SEPT6(23157); CUL4B(8450); FAM58A |
| | | (92002); |
| hsa-miR-92b | 819; 822; 823; | RAP1A(5906); FASLG(356); IL19(29949); CIAS1(114548); TESK2(10420); |
| | 826; 827; | FAF1(11124); MCL1(4170); DEDD(9191); BIRC6(57448); PRKCE(5581); |
| | 829; 830; | BCL2L11(10018); RIF1(55183); CASP10(843); ALS2CR19(117583); PIP5K3 |
| | 831; 832; 833; | (200576); COL4A3(1285); CARD12(58484); EIF2AK2(5610); TIA1(7072); |
| | 834; 835; | ANAPC1(64682); MCM6(4175); TLK1(9874); PAX3(5077); SYN2(6854); |
| | 836; 837; | PCAF(8850); CTNNB1(1499); APPL(26060); MCM2(4171); FAIM(55179); |
| | 838; 839; 8 | FXR1(8087); STAG1(10274); TFDP2(7029); ATR(545); HD(3064); EREG( |

Table 4-154

| | | |
|---|---|---|
| | 40; 842; 843; | 2069); FGF2(2247); FLJ20273(54502); CASP6(839); CARD6(84674); MRPS30 |
| | 846; 847; | (10884); GZMA(3001); RAD17(5884); MAN2A1(4124); RAD50(10111); |
| | 848; 850; 851; | PCDHB13(56123); PRKAA1(5562); KIAA1961(96459); IRF1(3659); FGF1 |
| | 12; 13; 818; | (2246); E2F3(1871); FOXO3A(2309); SASH1(23328); BAK1(578); DST(667); |
| | 820; 821; | CCNC(892); LATS1(9113); PDCD2(5134); HDAC9(9734); RINT1(60561); |
| | 279; 294; | HBP1(26959); SOSTDC1(25928); CYCS(54205); CDK6(1021); TACC1 |
| | 824; 825; 280; | (6867); FZD6(8323); TNFRSF10B(8795); ADRA1A(148); CLU(1191); CCNE2 |
| | 295; 828281; | (9134); RAD21(5885); TNFRSF11B(4982); TRAF1(7185); TSC1(7248); |
| | 296; | PTEN(5728); BTRC(8945); PARD3(56288); DLG5(9231); C10orf46(143384); |
| | 282; 297; 283; | IFITM1(8519); STIM1(6786); HIPK3(10114); BIRC3(330); CUL5(8065); |
| | 298; 284; | CDKN1C(1028); DKK3(27122); FZD4(8322); SESN3(143686); ZW10(9183); |
| | 299; 285; | DYRK2(8445); MDM2(4193); NUDT4(11163); GAS2L3(283431); FZD10 |
| | 300; 286; $$301; | (11211); CD69(969); BCAT1(586); KRAS(3845); STAT2(6773); BTG1(694); |
| | 287; 302; | DIP13B(55198); TMEM119(338773); SCARB1(949); HSMPP8(54737); |
| | 288; 303; | ING1(3621); CUL4A(8451); LATS2(26524); CGRRF1(10668); TRAF3(7187); |
| | 204; 841; | STRN3(29966); LTBP2(4053); NEK9(91754); MOAP1(64112); PPP1R13B |
| | 289; 841; 845; | (23368); SPN(6693); PAPD5(64282); RBL2(5934); NOL3(8996); CTCF |
| | 290; 305; | (10664); CDT1(81620); EXOSC6(118460); PHLPPL(23035); PAFAH1B1(5048); |
| | 291; 849; | NLK(51701); NF1(4763); CDC6(990); PPM1D(8493); MAP2K6(5608); |
| | 292; 293; | CETN1(1068); DCC(1630); ESCO1(114799); BCL2(596); FZR1(51343); TNFSF9 |
| | | (8744); AXL(558); APOE(348); ZNF530(348327); MCM8(84515); CTNNBL1 |
| | | (56259); PARD6B(84612); AURKA(6790); CTCFL(140690); DIDO1 |
| | | (11083); APP(351); SNF1LK(150094); CDC45L(8318); NF2(4771); PGEA1 |
| | | (25776); TOB2(10766); PCTK1(5127); TAF1(6872); BIRC4(331); PDCD8 |
| | | (9131); |
| hsa-miR-449b | 917; 770; 771; | VWA1(64856); H6PD(9563); NBL1(4681); PLA2G2F(64600); SESN2(83667); |
| | 772; 773; | HDAC1(3065); MACF1(23499); CDC20(991); PLK3(1263); BCL9(607); |
| | 774; 775; | HCN3(57657); PEA15(8682); FASLG(356); NAV1(89796); ADORA1(134); |
| | 776; 90; 918; | MDM4(4194); CIAS1(114548); TNFRSF14(8764); TNFRSF9(3604); CTNNBIP1 |
| | 2255; | (56998); DFFA(1676); FRAP1(2475); CASP9(842); ZBTB17(7709); |
| | | RCC2(55920); C1orf183(55924); NOTCH2(4853); PYGO2(90780); TP53BP2 |
| | | (7159); KCNK3(3777); BIRC6(57448); PRKCE(5581); SEMA4F(10505); |
| | | HK2(3099); MAL(4118); BRRN1(23397); TAIP-2(80034); CFLAR(8837); |
| | | FZD7(8324); CD28(940); ASB1(51665); TGFA(7039); EDAR(10913); PAX3 |
| | | (5077); CUL3(8452); HDAC4(9759); DTYMK(1841); FANCD2(2177); VHL |
| | | (7428); DLEC1(9940); MCM2(4171); CLDN18(51208); SMC4L1(10051); CMYA1 |
| | | (165904); UCN2(90226); NEK4(6787); RAB43(339122); SLCO2A1 |
| | | (6578); PIK3CB(5291); TFDP2(7029); HD(3064); ANAPC4(29945); EREG |
| | | (2069); SEPT11(55752); NFKB1(4790); FGF2(2247); LETM1(3954); MXD4 |
| | | (10608); SNCA(6622); UNC5C(8633); LEF1(51176); CASP6(839); KIAA1909 |
| | | (153478); CCNB1(891); CSNK1G3(1456); TCF7(6932); NDST1(3340); |
| | | CPLX2(10814); UNC5A(90249); TPPP(11076); PRLR(5618); BIRC1(4671); |
| | | SEMA6A(57556); IRF1(3659); PPP2CA(5515); CDC23(8697); CSNK1A1 |
| | | (1452); DUSP1(1843); E2F3(1871); TCF19(6941); CSNK2B(1460); MAPK13 |
| | | (5603); PARC(23113); VEGF(7422); IL17(3605); TNFAIP3(7128); HECA |
| | | (51696); NEDD9(4739); DHX16(8449); DAXX(1616); CUL7(9820); DST |
| | | (667); ATG5(9474); FRK(2444); PLAGL1(5325); DLL1(28514); FZD9(8326); |
| | | BCAP29(55973); CASP2(835); MAD1L1(8379); TRIAD3(54476); SOSTDC1 |
| | | (25928); CYCS(54205); CARD4(10392); ELMO1(9844); YWHAG(7532); |
| | | CDK6(1021); MCM7(4176); ZC3HC1(51530); MGC5242(78996); HIPK2 |
| | | (28996); KIAA0773(9715); POLR3D(661); TNFRSF10C(8794); UNC5D(137970); |
| | | SULF1(23213); E2F5(1875); PINX1(54984); C8orf13(83648); LZTS1 |
| | | (11178); TNFRSF10B(8795); TRIM35(23087); DUSP4(1846); MOS(4342); |
| | | CCNE2(9134); TP53INP1(94241); RAD21(5885); EXT1(2131); JAK2(3717); |
| | | UNC13B(10497); RECK(8434); C9orf47(286223); GADD45G(10912); |
| | | FGD3(89846); ABL1(25); TAF1L(138474); GAS1(2619); SEMA4D(10507); |
| | | PTCH(5727); TRAF1(7185); CIZ1(25792); ZMYND11(10771); DKK1(22943); |
| | | SGPL1(8879); UNC5B(219699); FRAT1(10023); LZTS2(84445); BTRC |
| | | (8945); PDCD11(22984); BAG3(9531); DOCK1(1793); IL2RA(3559); DLG5 |
| | | (9231); SFRP5(6425); FBXW4(6468); FAM26C(255022); DCLRE1A(9937); |
| | | MKI67(4288); WEE1(7465); TSPAN18(90139); F2(2147); INCENP(3619); |
| | | DPF2(5977); SIPA1(6494); RPS6KB2(6199); ATM(472); CD3E(916); LOH11CR2A |
| | | (4013); EI24(9538); DUSP8(1850); IGF2(3481); CDKN1C(1028); |
| | | RHOG(391); BAD(572); CDCA5(113130); MAP3K11(4296); RELA(5970); |
| | | FOSL1(8061); CLCF1(23529); PPP1CA(5499); FZD4(8322); BTG4(547 |

Table 4-155

| | | |
|---|---|---|
| | | 66); PPP2R1B(5519); PCSK7(9159); BCL9L(283149); LTBR(4055); TNFRSF7 |
| | | (939); BCL2L14(79370); CDKN1B(1027); IAPP(3375); ESPL1(9700); |
| | | PDE1B(5153); TNFRSF1A(7132); NOL1(4839); BCAT1(586); HDAC7A(51564); |
| | | MCRS1(10445); FMNL3(91010); FAIM2(23017); C12orf22(81566); |
| | | DDIT3(1649); PCTK2(5128); PPP1CC(5501); ANAPC5(51433); DIABLO |
| | | (56616); VPS37B(79720); MPHOSPH9(10198); CHFR(55743); RFP2(10206); |
| | | NEK3(4752); GZMH(2999); STRN3(29966); EGLN3(112399); NFKBIA |
| | | (4792); LTBP2(4053); TGFB3(7043); CHES1(1112); MOAP1(64112); JAG2 |
| | | (3714); THSD4(79875); CSK(1445); IGF1R(3480); NDN(4692); BMF(90427); |
| | | DAPK2(23604); CSNK1G1(53944); BCL2A1(597); FLJ22795(80154); AP3S2 |
| | | (10239); FLJ36208(283948); RAB40C(57799); TRAF7(84231); KREMEN2 |
| | | (79412); DNASE1(1773); PLK1(5347); CARD15(64127); CCL22(6367); |
| | | KATNB1(10300); NOL3(8996); CTCF(10664); VPS4A(27183); CDT1 |
| | | (81620); AFG3L1(172); GAS8(2622); PKMYT1(9088); XYLT1(64131); ERN2 |
| | | (10595); SEPT1(1731); C16orf58(64755); DNAJA2(10294); FTS(64400); |
| | | SMPD3(55512); RUTBC1(9905); GSG2(83903); ALOX12(239); TNFSF12 |
| | | (8742); ALOX15B(247); NTN1(9423); SARM1(23098); TRAF4(9618); NF1 |
| | | (4763); LIG3(3980); STARD3(10948); FZD2(2535); MAPT(4137); NGFR(4804); |
| | | PRKCA(5578); CDK3(1018); BIRC5(332); ANAPC11(51529); GPS1(2873); |
| | | MNT(4335); NALP1(22861); GPS2(2874); TP53(7157); VAMP2(6844); |
| | | FLOT2(2319); MPP2(4355); ERN1(2081); SOCS3(9021); CABLES1(91768); |
| | | MAPRE2(10982); KIAA0427(9811); SERPENB2(5055); DSC3(1825); |
| | | FZR1(51343); HMG20B(10362); CHAF1A(10036); CASP14(28581); PIK3R2 |
| | | (5296); CCNE1(898); LGALS7(3963); SAMD4B(55095); MAP3K10(4294); |
| | | AXL(558); FOSB(2354); GRLF1(2909); SPHK2(56848); BAX(581); PPP2R1A |
| | | (5518); NALP2(55655); FLJ45684(400666); TNFSF14(8740); CDC37 |
| | | (11140); DPF1(8193); DEDD2(162989); ERCC2(2068); PPP1R13L(10848); |
| | | LIG1(3978); LOC284296(284296); TRIB3(57761); STK35(140901); PLCB1 |
| | | (23236); SRC(6714); MYBL2(4605); CD40(958); PARD6B(84612); ProSAPiP1 |
| | | (9762); CTCFL(140690); CABLES2(81928); DIDO1(11083); USP16 |
| | | (10600); SON(6651); PDXK(8566); BCL2L13(23786); SEPT5(5413); NF2 |
| | | (4771); MTP18(51537); PGEA1(25776); TNRC6B(23112); EP300(2033); SEPT3 |
| | | (55964); BIK(638); MAPK8IP2(23542); BID(637); MAPK1(5594); PPM1F |
| | | (9647); IL2RB(3560); SSTR3(6753); CARD10(29775); TOB2(10766); |
| | | POLR3H(171568); SMC1L2(27127); HDAC6(10013); CCNB3(85417); MAGEH1 |
| | | (28986); TAF1(6872); IKBKG(8517); SMC1L1(8243); HCFC1(3054); |
| hsa-miR-557 | 925; 106; | DFFB(1677); KHDRBS1(10657); MACF1(23499); NFYC(4802); CD2(914); |
| | | CKS1B(1163); DAP3(7818); IL19(29949); CIAS1(114548); TNFRSF9(3604); |
| | | RCC2(55920); BCL10(8915); NOTCH2(4853); DUSP10(11221); TP53BP2 |
| | | (7159); RHOB(388); PPP1CB(5500); BIRC6(57448); PRKCE(5581); STEAP3 |
| | | (55240); RAB3GAP1(22930); RIF1(55183); ZAK(51776); TIA1(7072); |
| | | ANAPC1(64682); MGC4268(83607); SCN3A(6328); SCN1A(6323); BARD1 |
| | | (580); RBMS3(27303); MCM2(4171); KNG1(3827); TP73L(8626); OXTR |
| | | (5021); TADA3L(10474); PIK3CB(5291); CD38(952); LEF1(51176); PDCD6 |
| | | (10016); APC(324); RAD50(10111); TCF7(6932); PRLR(5618); PRKAA1 |
| | | (5562); MCC(4163); CDC23(8697); RNF130(55819); E2F3(1871); ZNF192 |
| | | (7745); TCF19(6941); CSNK2B(1460); CDC5L(988); IL17(3605); PTP4A1 |
| | | (7803); FOXO3A(2309); IHPK3(117283); DST(667); BACH2(60468); MAP3K5 |
| | | (4217); LATS1(9113); AHR(196); HBP1(26959); CUL1(8454); SOSTDC1 |
| | | (25928); INHBA(3624); LOC401357(401357); CDK6(1021); ZC3HC1(51530); |
| | | PTN(5764); UNC5D(137970); UBE2V2(7336); BIN3(55909); TNFRSF10D |
| | | (8793); TNFRSF10A(8797); TRIM35(23087); CLU(1191); TNFRSF11B |
| | | (4982); CDC37L1(55664); INVS(27130); TRAF2(7186); CDKN2B(1030); |
| | | BAG1(573); PTCH(5727); TSC1(7248); ANAPC2(29882); DKK1(22943); |
| | | SIRT1(23411); FAS(355); FRAT1(10023); DOCK1(1793); IL2RA(3559); FRAT2 |
| | | (23401); TIAL1(7073); MKI67(4288); BNIP3(664); TEAD1(7003); HIPK3 |
| | | (10114); EXT2(2132); INCENP(3619); LGALS12(85329); DPF2(5977); |
| | | SIPA1(6494); ATM(472); CHEK1(1111); CDKN1C(1028); EIF4G2(1982); |
| | | DKK3(27122); HRASLS3(11145); CDCA5(113130); FZD4(8322); CCND2 |
| | | (894); NTF3(4908); TNFRSF7(939); CNAP1(9918); MDM2(4193); SOCS2 |
| | | (8835); KNTC1(9735); TMEM132B(114795); FZD10(11211); BCAT1(586); |
| | | BTG1(694); HRK(8739); CHFR(55743); FGF9(2254); RB1(5925); RFP2(10206); |
| | | ING1(3621); CUL4A(8451); FOXO1A(2308); PNN(5411); GPR65(8477); |
| | | MAMDC1(161357); BAG5(9529); FGF7(2252); IGF1R(3480); AVEN(5 |

Table 4-156

| | | |
|---|---|---|
| | | 7099); TRAF7(84231); CCNF(899); DNASE1(1773); CARD15(64127); AXIN1 |
| | | (8312); GSPT1(2935); PAFAH1B1(5048); NF1(4763); LIG3(3980); MAPT |
| | | (4137); RPS6KB1(6198); CDK3(1018); ANAPC11(51529); MNT(4335); P2RX1 |
| | | (5023); NALP1(22861); SPAG5(10615); CBX4(8535); TBC1D16(125058); |
| | | DCC(1630); PARD6G(84552); CHAF1A(10036); ARHGEF1(9138); DPF1 |
| | | (8193); PLCB1(23236); RAB22A(57403); PCNA(5111); RBL1(5933); CTCFL |
| | | (140690); SYCP2(10388); SON(6651); SLC5A3(6526); APP(351); CRYZL1 |
| | | (9946); EP300(2033); PPM1F(9647); AGTR2(186); BIRC4(331); STAG2 |
| | | (10735); RP6-213H19.1(51765); DDX26B(203522); FIGF(2277); ARHGEF6 |
| | | (9459); |
| hsa-miR-563 | 916; 81; 760; | TP73(7161); TESK2(10420); LPGAT1(9926); ITPKB(3707); BIRC6(57448); |
| | | GKN1(56287); CD28(940); BIN1(274); ERCC3(2071); FRZB(2487); PAX3 |
| | | (5077); PDCD6IP(10015); KNG1(3827); OPA1(4976); ATR(545); KLHL6 |
| | | (89857); HD(3064); ADAM29(11086); MRPS30(10884); CSNK1G3(1456); RAD50 |
| | | (10111); MCC(4163); ATG12(9140); SEMA6A(57556); CDC25C(995); |
| | | TNF(7124); IL17(3605); PTP4A1(7803); SASH1(23328); TNFRSF21(27242); |
| | | FRK(2444); LATS1(9113); CUL1(8454); TRIAD3(54476); CARD4(10392); |
| | | ELMO1(9844); HIPK2(28996); UNC5D(137970); SLC30A8(169026); |
| | | ADRA1A(148); PLAG1(5324); JAK2(3717); DAPK1(1612); NCBP1(4686); |
| | | ABL1(25); CDKN2B(1030); PTCH(5727); TNFSF8(944); C10orf9(219771); |
| | | BTRC(8945); TCF7L2(6934); DOCK1(1793); CXCL12(6387); DLG5(9231); |
| | | EIF4G2(1982); FZD4(8322); TEGT(7009); RBMS2(5939); ANKRD13(88455); |
| | | KNTC1(9735); TNFRSF19(55504); ATP8A2(51761); BRCA2(675); CUL4A |
| | | (8451); TFDP1(7027); FOXO1A(2308); DZIP1(22873); VASH1(22846); |
| | | TRAF3(7187); EXOC5(10640); JAG2(3714); RNF111(54778); UBE3A(7337); |
| | | KATNB1(10300); AXIN1(8312); GSPT1(2935); MAPK7(5598); NLK |
| | | (51701); MAPT(4137); TLK2(11011); PRKCA(5578); CARD14(79092); MPO |
| | | (4353); TMC6(11322); CETN1(1068); BCL2(596); UHRF1(29128); TNFSF9 |
| | | (8744); ARHGEF1(9138); BCL3(602); FOSB(2354); NALP2(55655); TNFSF14 |
| | | (8740); ERCC2(2068); TRIB3(57761); C20orf91(284800); USP16(10600); |
| | | KREMEN1(83999); MTP18(51537); EP300(2033); CARD10(29775); |
| | | PCTK1(5127); CCNB3(85417); CUL4B(8450); |
| hsa-miR-613 | 803; 1; 2; 130; | CDC42(998); RCC1(1104); RBBP4(5928); CDC20(991); KIAA0467(23334); |
| | 140; 131; | SH3GLB1(51100); PROK1(84432); CD2(914); BCL9(607); BNIPL(149428); |
| | 141; 142; 132; | CKS1B(1163); PEA15(8682); UHMK1(127933); IL24(11009); C1orf95 |
| | 143; 133; | (375057); YARS(8565); CLSPN(63967); TESK2(10420); SASS6(163786); NOTCH2 |
| | 144; 134; | (4853); PYGO2(90780); DEDD(9191); RHOB(388); BIRC6(57448); |
| | 135; 145; 136; | PRKCE(5581); KCNIP3(30818); TAIP-2(80034); CASP10(843); CASP8(841); |
| | 146; 137; | FZD7(8324); CD28(940); COL4A3(1285); SEPT2(4735); YWHAQ(10971); |
| | 147; 138; | CARD12(58484); ANAPC1(64682); TLK1(9874); FRZB(2487); NCKAP1 |
| | 139; | (10787); PAX3(5077); HDAC4(9759); FANCD2(2177); VHL(7428); RBM5 |
| | | (10181); GNL3(26354); FAIM(55179); KNG1(3827); CIDEC(63924); RAF1 |
| | | (5894); UBE1C(9039); PIK3CB(5291); ATR(545); CCNG2(901); CYP2U1 |
| | | (113612); APBB2(323); UNC5C(8633); SFRP2(6423); NIPBL(25836); RNUXA |
| | | (51808); DAP(1611); MCC(4163); SEMA6A(57556); IRF1(3659); CDC25C |
| | | (995); PFDN1(5201); VEGF(7422); UST(10090); NEDD9(4739); CCNC |
| | | (892); BCLAF1(9774); LATS1(9113); PDCD2(5134); AHR(196); ADCY1 |
| | | (107); EGFR(1956); HBP1(26959); CNTNAP2(26047); CUL1(8454); PMS2 |
| | | (5395); ELMO1(9844); IGFBP3(3486); CDK6(1021); BNIP3L(665); FZD3(7976); |
| | | BAG4(9530); SULF1(23213); E2F5(1875); FZD6(8323); EBAG9(9166); |
| | | WISP1(8840); PINX1(54984); TNFRSF10B(8795); TRIM35(23087); SFRP1 |
| | | (6422); STK3(6788); UNC13B(10497); CDK9(1025); CDKN2B(1030); |
| | | SEMA4D(10507); PTCH(5727); TNFSF15(9966); TNFSF8(944); TNKS2 |
| | | (80351); LZTS2(84445); BTRC(8945); TCF7L2(6934); BAG3(9531); DOCK1 |
| | | (1793); MKI67(4288); WEE1(7465); GAS2(2620); HIPK3(10114); API5(8539); |
| | | LRP5(4041); LOH11CR2A(4013); BDNF(627); WT1(7490); SHANK2 |
| | | (22941); FZD4(8322); SESN3(143686); ZW10(9183); BCL9L(283149); CCND2 |
| | | (894); TNFRSF7(939); CRADD(8738); OAS2(4939); BCAT1(586); KRAS |
| | | (3845); CDK4(1019); ARPC3(10094); MPHOSPH9(10198); CHFR(55743); |
| | | HSMPP8(54737); RB1(5925); LATS2(26524); BCL2L2(599); KIAA0323 |
| | | (23351); TXNDC(81542); TRAF3(7187); CDKL1(8814); DLG7(9787); LTBP2 |
| | | (4053); KLF13(51621); BUB1B(701); PTPLAD1(51495); PAQR5(54852); |
| | | IGF1R(3480); AVEN(57099); BMF(90427); BNIP2(663); CSNK1G1(53944); |
| | | CYLD(1540); KATNB1(10300); CTCF(10664); NFATC3(4775); AXIN1 |
| | | (8312); MAPK3(5595); FTS(64400); TERF2(7014); AP1G1(164); PAFAH |

Table 4-157

1B1(5048); MAPK7(5598); CCL2(6347); LIG3(3980); LASP1(3927); NGFR
(4804); TLK2(11011); PRKCA(5578); SPHK1(8877); MNT(4335); GAS7(8522);
BRCA1(672); PTRH2(51651); C17orf62(79415); CABLES1(91768); MAPK4
(5596); PHLPP(23239); ROCK1(6093); ZNF24(7572); BCL2(596); CHAF1A
(10036); ARHGEF18(23370); PSMD8(5714); AXL(558); APOE(348);
FOSB(2354); SPHK2(56848); NALP2(55655); DAPK3(1613); ZNF571
(51276); DPF1(8193); CARD8(22900); KLK10(5655); CEP250(11190); STK4
(6789); BAK1(578); AURKA(6790); CTCFL(140690); SON(6651); BCL2L13
(23786); SMARCB1(6598); GAS2L1(10634); SEPT3(55964); MAPK1(5594);
PPM1F(9647); CARD10(29775); SMC1L2(27127); CCNB3(85417); ARSD
(414); BTK(695); SEPT6(23157); HCFC1(3054); G6PD(2539);

As a method of expressing a nucleic acid such as a miRNA in cells in the present invention, a method using a nucleic acid that causes the expression of a gene that encodes the miRNA and the like when introduced into the cells can be mentioned. As the nucleic acid, a DNA, an RNA, or a nucleotide analogue, as well as a chimeric molecule thereof, or a derivative of the nucleic acid can also be used. Specifically, the nucleic acid can be designed in the same way as with Pre-miR™ miRNA Precursor Molecules (manufactured by Ambion) or miRIDIAN microRNA Mimics (manufactured by GE Healthcare), and the nucleic acid such as the miRNA can be expressed in cells. Any method can be used to express a miRNA, as far as it allows the miRNA to be finally formed in the cells; for example, (1) a method wherein a single-stranded RNA that is a precursor of the miRNA is introduced, as well as (2) a method wherein an RNA consisting of a 100% matched double-strand consisting of the miRNA as it is and a complementary strand for the miRNA, is introduced, and (3) a method wherein a double-stranded RNA assumed to have resulted from cleavage of a miRNA with a Dicer is introduced, can be mentioned. As examples of commercial products based on these methods, miCENTURY OX Precursor (manufactured by B-Bridge), miCENTURY OX siMature (manufactured by B-Bridge), and miCENTURY OX miNatural (manufactured by B-Bridge), respectively, can be mentioned.

The method of synthesizing a nucleic acid to be used in the present invention is not particularly limited; the same can be produced by a method using a publicly known chemical synthesis, or an enzymatic transcription method and the like. As methods using a publicly known chemical synthesis, the phosphoroamidite method, the phosphorothioate method, the phosphotriester method and the like can be mentioned; for example, the same can be synthesized using the ABI3900 high throughput nucleic acid synthesizer (manufactured by Applied Biosystems). As an enzymatic transcription method, transcription with a plasmid or DNA having a desired nucleotide sequence as the template using a typical phage RNA polymerase, for example, T7, T3, or SP6RNA polymerase, can be mentioned.

As an example of a method of screening a substance that promotes or suppresses the expression or a function of a nucleic acid used in the present invention by means of the nucleic acid, a method can be mentioned wherein a vector that expresses the nucleic acid is introduced into cells, and a substance that promotes or suppresses the expression or a function of a mRNA having a target nucleotide sequence therefor is screened for.

A pharmaceutical with a nucleic acid to be used in the present invention as an active ingredient can be used to diagnose or treat a disease caused by an abnormality of cell proliferation and the like, tissue hyperplasia and the like. In addition, a nucleic acid can also be used as an agent for suppressing or promoting cell proliferation. Here, an abnormality of cell proliferation refers to a condition wherein cells are proliferating at a rate that is not a normal proliferation rate in a living organism.

As diseases caused by a cell proliferation abnormality, tissue hyperplasia and the like, specifically, cancers, arteriosclerosis, rheumatoid arthritis, prostatic hyperplasia, blood vessel restenosis after percutaneous transvascular coronary angioplasty, fibroid lung, glomerulonephritis, autoimmune diseases and the like, preferably cancer, can be mentioned.

Hereinafter, the present invention is described in detail.

1. Method of Detecting the Expression of a Nucleic Acid Such as miRNA or miRNA Precursor Expressed in Cancer Cells
(1-1) Identification of miRNA

Whether or not the small RNA sequence is a miRNA can be determined on the basis of whether or not the criteria described in RNA, 9, 277-279 (2003) are met. For example, in cases where the small RNA was newly acquired and the nucleotide sequence thereof was determined, this can be performed as described below.

A surrounding genomic sequence wherein a DNA sequence corresponding to the nucleotide sequence of the small RNA acquired is extended by about 50 nt toward the 5′ terminal side and the 3′ terminal side, respectively, is acquired, and the secondary structure of the RNA expected to be transcribed from the genomic sequence is predicted. If the result shows that a hairpin structure is present and the nucleotide sequence of the small RNA is located in one chain of the hairpin, the small RNA can be judged to be a miRNA. Genomic sequences are open to the general public, and are available from, for example, UCSC Genome Bioinformatics (http://genome.ucsc.edu/). For prediction of secondary structures, various programs are open; for example, RNAfold [Nucleic Acids Research 31, 3429-3431 (2003)], Mfold [Nucleic Acids Research 31, 3406-3415 (2003)] and the like can be used. Existing miRNA sequences are registered in the database called miRBase in Sanger Institute, UK; whether or not a miRNA is identical to an existing miRNA can be determined on the basis of whether or not the sequence thereof is identical to one of the sequences listed therein.

### (1-2) Acquisition of Sequence Information on Small RNA

Total RNA is extracted from a human cancer cell line or a cancer patient-derived sample, and a small RNA comprising a miRNA expressed in cancer cells or cancer tissue can be acquired using the total RNA as described below.

As a method of acquiring a small RNA, specifically, a method wherein separation and cutting out of a small RNA by 15% polyacrylamide gel electrophoresis as described in Genes & Development 15, 188-200 (2000), can be mentioned. Then, 5′ terminal dephosphorylation, 3′-adapter ligation, phosphorylation, 5′-adapter ligation, reverse transcription, PCR amplification, concatemerization, and ligation to a vector are performed sequentially, thereafter the small RNA is cloned, and the nucleotide sequence of the clone is determined. Alternatively, for example, a method wherein 5′-adenylation 3′-adapter ligation, 5′-adapter ligation, reverse transcription, PCR amplification, concatemerization, and ligation to a vector are performed sequentially, thereafter the small RNA is cloned, and the nucleotide sequence of the clone is also determined, as described in Science 294, 858-862 (2001).

Alternatively, 5′ terminal dephosphorylation, 3′-adapter ligation, phosphorylation, 5′-adapter ligation, reverse transcription, PCR amplification, and ligation to a microbead vector are performed sequentially, thereafter the small RNA is cloned, and the nucleotide sequence of the microbeads is read to determine the nucleotide sequence, whereby nucleotide sequence information of the small RNA can also be acquired, as described in Nucleic Acids Research 34, 1765-1771 (2006).

As another method for acquiring a small RNA, a method using a small RNA Cloning Kit (manufactured by Takara Bio Inc.) can be mentioned.

(1-3) Method of Detecting the Expression Level of a Nucleic Acid Such as a miRNA

As examples of methods of detecting the expression level of a nucleic acid such as miRNA, a miRNA precursor and the like include (1) Northern hybridization, (2) dot blot hybridization, (3) in situ hybridization, (4) quantitative PCR, (5) differential hybridization, (6) microarray, (7) ribonuclease protection assay and the like.

The Northern blot method is a method wherein a sample-derived RNA is separated by gel electrophoresis, then transferred to a solid support such as a nylon filter, and an appropriately-labeled probe is prepared on the basis of the nucleotide sequence of a nucleic acid, and hybridization and washing are performed, whereby a band specifically bound to the nucleic acid is detected; specifically, for example, this method can be performed as described in Science 294, 853-858 (2001) and the like.

A labeled probe can be prepared by incorporating a radioisotope, biotin, digoxigenin, a fluorescent group, a chemiluminescent group and the like in a DNA, RNA, or LNA having a sequence complementary to the nucleotide sequence of a nucleic acid to be used in the present invention by a method, for example, nick translation, random priming or 5′-terminal phosphorylation. Because the amount of labeled probe bound reflects the expression level of the nucleic acid, the expression level of the nucleic acid can be quantified by quantifying the amount of labeled probe bound. Electrophoresis, membrane transfer, probe preparation, hybridization, and nucleic acid detection can be achieved by a method described in Molecular Cloning, 3rd edition [Cold Spring Harbor Press, (2001) Cold Spring Harbor, N.Y.].

Dot blot hybridization is a method wherein an RNA extracted from a tissue or a cell is spotted in dot forms and immobilized on a membrane, and hybridized with a labeled polynucleotide to be a probe, and an RNA that specifically hybridizes with the probe is detected. The probe used may be the same as that used for Northern hybridization. RNA preparation, RNA spotting, hybridization, and RNA detection can be achieved by a method described in Molecular Cloning, 3rd edition.

In situ hybridization is a method wherein a paraffin-embedded or cryostat-treated section of a tissue acquired from a living organism, or a cell fixed, is used as a sample and subjected to steps for hybridization with a labeled probe and washing, and the distribution and localization of a nucleic acid in the tissue or cell are examined by microscopic examination [Methods in Enzymology, 254, 419 (1995)]. The probe used may be the same as that used for Northern hybridization. Specifically, a miRNA can be detected in accordance with a method described in Nature Method 3, 27 (2006).

In quantitative PCR, a cDNA synthesized from a sample-derived RNA using a primer for reverse transcription and a reverse transcriptase (hereunder, this cDNA is referred to as a sample-derived cDNA) is used for the measurement. As a primer for reverse transcription to be supplied for cDNA synthesis, a random primer or a specific RT primer and the like can be used. A specific RT primer refers to a primer having a sequence complementary to a nucleotide sequence corresponding to a nucleic acid and a genomic sequence therearound.

For example, a sample-derived cDNA is synthesized, after which a PCR is performed with this cDNA as the template, using a template-specific primer designed from a nucleotide sequence corresponding to a nucleic acid such as miRNA or miRNA precursor and a genomic sequence therearound, or from a nucleotide sequence corresponding to a primer for reverse transcription, to amplify a cDNA fragment containing the nucleic acid, and the amount of the nucleic acid contained in the sample-derived RNA is detected from the number of cycles for reach to a given amount of the fragment. As the template-specific primer, an appropriate region corresponding to a nucleic acid and a genomic sequence therearound is selected, and a pair of a DNA or LNA consisting of a sequence of 15 to 40 nucleotides at the 5′ terminus, preferably 20 to 30 nucleotides of the nucleotide sequence of the region, and a DNA or LNA consisting of a sequence complementary to a sequence of 15 to 40 nucleotides at the 3′ terminus, preferably 20 to 30 nucleotides can be used. Specifically, this can be performed in accordance with a method described in Nucleic Acids Research, 32, e43 (2004) and the like.

Alternatively, as the primer for reverse transcription to be supplied for cDNA synthesis, a specific RT primer having a stem-loop structure can also be used. Specifically, this can be performed using a method described in Nucleic Acid Research, 33, e179 (2005), or TaqMan MiRNA Assays (manufactured by Applied Biosystems).

As still another method of synthesizing a sample-derived cDNA, a polyA sequence is added to a sample-derived RNA by means of polyA polymerase, and a nucleotide sequence comprising an oligodT sequence is used as a primer for reverse transcription, whereby a reverse transcription reaction can be performed. Specifically, this can be performed using the miScript System (manufactured by QIAGEN) or the QuantiMir RT Kit (manufactured by System Biosciences). Furthermore, by hybridizing a sample-derived RNA or cDNA to a DNA or LNA corresponding to a nucleotide sequence comprising at least one or more of a nucleic acid immobilized on a substrate such as a filter, glass slide, or silicone having, and performing washing, a change in the amount of the nucleic acid can be detected.

As such methods based on hybridization, methods using differential hybridization [Trends Genet., 7, 314 (1991)] or a microarray [Genome Res., 6, 639 (1996)] can be mentioned. Both methods enable accurate detection of a difference in the amount of a nucleic acid between a control sample and a target sample by immobilizing an internal control, such as a nucleotide sequence corresponding to U6RNA, on a filter or a substrate. Also, by synthesizing labeled cDNAs using differently labeled dNTPs (mixtures of dATP, dGTP, dCTP, and dTTP) on the basis of RNAs derived from a control sample and a target sample, and simultaneously hybridizing the two labeled cDNAs to a single filter or a single substrate, accurate quantitation of a nucleic acid can be performed. Furthermore, quantitation of a nucleic acid can also be performed by directly labeling and hybridizing an RNA derived from a control sample and/or a target sample. For example, a miRNA can be detected using a microarray described in Proc. Natl. Acad. Sci. USA, 101, 9740-9744 (2004), Nucleic Acid Research, 32, e188 (2004), RNA, 13, 151-159 (2007) and the like. Specifically, a miRNA can be detected or quantified in the same manner as mirVana miRNA Bioarray (manufactured by Ambion) and miRNA microarray kit (manufactured by Agilent Technologies).

In ribonuclease protection assay, first, a promoter sequence such as the T7 promoter or the SP6 promoter is bound to the 3′ terminus of a nucleotide sequence corresponding to a nucleic acid or a genomic sequence therearound, and a labeled antisense RNA is synthesized with an in vitro transcription system using a labeled NTP (a mixture of ATP, GTP, CTP, and UTP) and an RNA polymerase. The labeled antisense RNA is bound to a sample-derived RNA to form an RNA-RNA hybrid, after which the hybrid is digested with ribonuclease A, which degrades single-stranded RNAs only. The digest is subjected to gel electrophoresis to detect or quantify an RNA fragment protected against the digestion by forming the RNA-RNA hybrid, as a nucleic acid. Specifically, the fragment can be detected or quantified using the mirVana miRNA Detection Kit (manufactured by Ambion).

### 2. Synthesis of Nucleic Acid

After a nucleic acid such as a miRNA or miRNA precursor expressed in cancer cells or cancer tissue, or exhibiting increased or decreased expression compared with normal tissue, has been identified as described in 1 above, not only an RNA, which is a polymer of a ribonucleotide, but also a DNA, which is a polymer of a deoxyribonucleotide, can be synthesized on the basis of the nucleotide sequences. For example, on the basis of the nucleotide sequence of the miRNA identified in 1 above, the nucleotide sequence of a DNA can be determined. The nucleotide sequence of a DNA corresponding to the nucleotide sequence of an RNA can be determined, without exception, by reading the U (uracil) contained in the sequence of the RNA as T (thymine). A polymer being a mixture of a ribonucleotide and a deoxyribonucleotide and a polymer comprising a nucleotide analogue and a derivative of a nucleic acid can also be synthesized in the same manner.

The method of synthesizing a nucleic acid is not particularly limited; the same can be produced by a method using a publicly known chemical synthesis, or an enzymatic transcription method and the like. As methods using a publicly known chemical synthesis, the phosphoroamidite method, the phosphorothioate method, the phosphotriester method and the like can be mentioned; for example, a nucleic acid can be synthesized using the ABI3900 high throughput nucleic acid synthesizer (manufactured by Applied Biosystems). As an enzymatic transcription method, a method by transcription with a plasmid or DNA having a desired nucleotide sequence as the template using a typical phage RNA polymerase, for example, T7, T3, or SP6RNA polymerase, can be mentioned.

3. Method of Detecting a Function of a Nucleic Acid Such as miRNA or miRNA Precursor

As a method of detecting a function of a nucleic acid such as a miRNA, a method can be mentioned wherein the function is detected on the basis of whether or not the translation of the mRNA having a target nucleotide sequence is suppressed.

MiRNAs are known to suppress the translation of a mRNA comprising a target nucleotide sequence thereof in the untranslated region on the 3′ side (3′UTR) [Current Biology, 15, R458-R460 (2005)]. Hence, a DNA wherein a target nucleotide sequence for the single-stranded RNA to be measured is inserted into the 3′UTR of an appropriate reporter gene expression vector is prepared and introduced into a host cell suitable for the expression vector, and the expression of the reporter gene is measured when the cell is allowed to express the single-stranded RNA, whereby whether or not a function of a miRNA is possessed can be determined.

The reporter gene expression vector may be any one, as far as it has a promoter upstream of a reporter gene, and is capable of expressing the reporter gene in the host cell. Any reporter gene can be used; for example, the firefly luciferase gene, the Renilla luciferase gene, the chloramphenicol acetyltransferase gene, the β-glucuronidase gene, the β-galactosidase gene, the β-lactamase gene, the aequorin gene, the green fluorescent protein gene, the DsRed fluorescence gene and the like can be utilized. As examples of reporter gene expression vectors having these properties, psiCHECK-1 (manufactured by Promega), psiCHECK-2 (manufactured by Promega), pGL3-Control (manufactured by Promega), pGL4 (manufactured by Promega), pRNAi-GL (manufactured by Takara Bio Inc.), pCMV-DsRed-Express (manufactured by CLONTECH) and the like can be mentioned. A single-stranded RNA can be expressed by the method described in 6 below.

As the function of a miRNA of a single-stranded RNA, specifically, detection can be achieved as described below. First, host cells are cultured on a multi-well plate and the like, and a reporter gene expression vector having a target sequence and a single-stranded RNA are expressed. Thereafter, reporter activity is measured both when the single-stranded RNA is and is not expressed, based on which the function as the miRNA of the single-stranded RNA can be detected.

4. Method of Detecting a Mutation of a Nucleic Acid Such as miRNA or miRNA Precursor

As a method of detecting a mutation of a nucleic acid such as a miRNA or miRNA precursor, a method can be used wherein a hetero-double-strand formed by hybridization of a normal type nucleic acid and a mutated type nucleic acid are detected.

As methods of detecting a hetero-double-strand, (1) detection of a hetero-double-strand by polyacrylamide gel electrophoresis [Trends genet., 7, 5 (1991)], (2) single-strand conformation polymorphism analysis [Genomics, 16, 325-332 (1993)], (3) chemical cleavage of mismatches (CCM) [Human Genetics (1996), Tom Strachan and Andrew P. Read, BIOS Scientific Publishers Limited], (4) enzymatic cleavage of mismatches [Nature Genetics, 9, 103-104 (1996)], (5) denatured gel electrophoresis [Mutat. Res., 288, 103-112 (1993)] and the like can be mentioned.

Detection of a hetero-double-strand by polyacrylamide gel electrophoresis is, for example, performed as described below. First, with a sample-derived DNA or a sample-derived cDNA as the template, and using a primer designed on the basis of a genomic nucleotide sequence comprising the nucleotide sequence of a nucleic acid, a fragment smaller than 200 by is amplified. Hetero-double-strands, if formed, are slower in mobility than mutation-free homo-double-strands, and can be detected as extra bands. In the case of search for a fragment smaller than 200 bp, almost any insertions, deletions, and substitutions of one or more nucleotides can be detected. It is desirable that hetero-double-strand analysis be performed using a single gel in combination with the single strand conformation analysis described below.

In single strand conformation polymorphism analysis (SSCP analysis), a DNA amplified as a fragment smaller than 200 by with a sample-derived DNA or sample-derived cDNA as the template, using a primer designed on the basis of a genomic nucleotide sequence comprising the nucleotide sequence of a nucleic acid, is denatured, after which it is electrophoresed in non-denatured polyacrylamide gel. By labeling the primer with an isotope or a fluorescent dye at the time of DNA amplification, or by silver-staining the non-labeled amplification product, the amplified DNA can be detected as a band. To clarify a difference from the wild type pattern, a control sample may be electrophoresed simultaneously, whereby a fragment with a mutation can be detected on the basis of a difference in mobility.

In chemical cleavage of mismatches (CCM method), a DNA fragment amplified with a sample-derived DNA or sample-derived cDNA as the template, using a primer designed on the basis of a genomic nucleotide sequence comprising the nucleotide sequence of a nucleic acid is hybridized to a labeled nucleic acid prepared by allowing a nucleic acid to incorporate an isotope or a fluorescent target, and treated with osmium tetraoxide to cleave one strand of the DNA at the mismatched portion, whereby a mutation can be detected. CCM is one of the most sensitive methods of detection, and can be applied to samples of kilobase length.

In place of osmium tetraoxide used above, T4 phage resolvase and an enzyme involved in mismatch repair in cells, such as endonuclease VII, and RNaseA may be used in combination to enzymatically cleave a mismatch. In denaturing gradient gel electrophoresis (DGGE method), a DNA amplified with a sample-derived DNA or sample-derived cDNA as the template, using a primer designed on the basis of a genomic nucleotide sequence comprising the nucleotide sequence of a nucleic acid is electrophoresed using a gel having a chemical denaturant density gradient or a temperature gradient. The DNA fragment amplified migrates in the gel to a position where it denatures to a single strand, and no longer migrates after the denaturation. Because the migration of the amplified DNA in the gel differs between the presence and absence of a mutation, the presence of the mutation can be detected. To increase the detection sensitivity, the addition of a poly (G:C) end to each primer is effective.

By directly determining and analyzing the nucleotide sequence of a sample-derived DNA or sample-derived cDNA, a mutation of a nucleic acid can also be detected.

5. Methods of Expressing a Nucleic Acid Such as miRNA or miRNA Precursor

Existing miRNA sequences and sequences of precursors thereof are registered with a database known as miRBase at the Sanger Institute in UK; nucleic acids such as miRNAs and miRNA precursors can be prepared by utilizing such sequences. Such nucleic acids can also be prepared using miRNA sequences acquired by the method described in 1.

A nucleic acid can be expressed by using a vector such that a nucleic acid is transcribed and hence biosynthesized when the vector is introduced into a cell. Specifically, by preparing a DNA fragment comprising a hairpin portion on the basis of the nucleotide sequence of a nucleic acid or a genomic nucleotide sequence comprising the foregoing nucleotide sequence, and inserting the fragment downstream of a promoter in the expression vector to construct an expression plasmids, and then introducing the expression plasmid into a host cell suitable for the expression vector, whereby a nucleic acid can be expressed.

As an expression vector, one capable of self-replication in the host cell, and capable of being incorporated in the chromosome, which comprises a promoter at a position enabling the transcription of a gene comprising the nucleotide sequence of a nucleic acid is used. The promoter may be any one, as far as it is capable of expressing in the host cell; for example, a RNA polymerase II (pol II) system promoter, a RNA polymerase III (pol III) system promoter being a U6RNA and H1RNA transcription system and the like can be mentioned. As examples of pol II system promoters, the promoter of the cytomegalovirus (human CMV) IE (immediate early) gene, the early promoter of SV40 and the like can be mentioned. As examples of expression vectors using them, pCDNA6.2-GW/miR (manufactured by Invitrogen), pSilencer 4.1-CMV (manufactured by Ambion) and the like can be mentioned. As pol III system promoters, U6RNA, H1RNA or tRNA gene promoters can be mentioned. As examples of expression vectors using them, pSINsi-hHl DNA (manufactured by Takara Bio Inc.), pSINsi-hU6 DNA (manufactured by Takara Bio Inc.), pENTR/U6 (manufactured by Invitrogen) and the like can be mentioned.

Alternatively, by inserting a gene comprising the nucleotide sequence of a nucleic acid into the downstream of the promoter in a viral vector to construct a recombinant viral vector, and introducing the vector into a packaging cell to produce a recombinant virus, the gene comprising the nucleic acid can be expressed.

The packaging cell may be any cell, as far as it is capable of supplementing a recombinant viral vector deficient in any one of the genes that encode the proteins necessary for the packaging of the virus with the lacked protein; for example, human kidney-derived HEK293 cells, mouse fibroblasts NIH3T3-derived cells and the like can be used. As the protein supplemented by the packaging cell, in the case of a retrovirus vector, proteins derived from a mouse retrovirus, such as gag, pol, and env, can be used; in the case of a lentivirus vector, proteins derived from a HIV virus, such as gag, pol, env, vpr, vpu, vif, tat, rev, and nef, can be used; in the case of an adenovirus vector, proteins derived from an adenovirus, such as E1A and E1B, can be used; in the case of an adeno-associated viral vector, proteins such as Rep (p5, p19, p40) and Vp(Cap), can be used.

In addition to using an expression vector, a nucleic acid to be used in the present invention can also be introduced directly into a cell, without using a vector. As the nucleic acid used in this technique, a DNA, an RNA, or a nucleotide analogue, as well as a chimeric molecule thereof, or a derivative of the nucleic acid can also be used. Specifically, in the same way as with Pre-miR™ miRNA Precursor Molecules (manufactured by Ambion) or miRIDIAN miRNA Mimics (manufactured by GE Healthcare), a nucleic acid such as miRNA or miRNA precursor can be expressed. Any method can be used to express a miRNA, as far as it allows the miRNA to be finally formed in the cells; for example, (1) a method wherein a single-stranded RNA that is a precursor of the miRNA is introduced, as well as (2) a method wherein an RNA consisting of a 100% matched double-strand consisting of the miRNA as it is and a complementary strand for the miRNA, is introduced, and (3) a method wherein a double-stranded RNA assumed to have resulted from cleavage of the miRNA with a Dicer is introduced, can be mentioned. As commercial products based on these methods, miCENTURY OX Precursor (manufactured by B-Bridge), miCENTURY OX siMature (manufactured by B-Bridge), and miCENTURY OX miNatural (manufactured by B-Bridge), respectively, can be mentioned.

6. Methods of Suppressing the Activity of a Nucleic Acid Such as miRNA or miRNA Precursor

The activation of a nucleic acid such as miRNA or miRNA precursor can be suppressed using an antisense technology [Bioscience and Industry, 50, 322 (1992); Kagaku, 46, 681 (1991), Biotechnology, 9, 358 (1992), Trends in Biotechnology, 10, 87 (1992), Trends in Biotechnology, 10, 152 (1992); Cell Technology, 16, 1463 (1997)], triple helix technology [Trends in Biotechnology, 10, 132 (1992)], ribozyme technology [Current Opinion in Chemical Biology, 3, 274 (1999), FEMS Microbiology Reviews, 23, 257 (1999), Frontiers in Bioscience, 4, D497 (1999), Chemistry & Biology, 6, R33 (1999), Nucleic Acids Research, 26, 5237 (1998), Trends In Biotechnology, 16, 438 (1998)], decoy DNA method [Nippon Rinsho—Japanese Journal of Clinical Medicine, 56, 563 (1998), Circulation Research, 82, 1023 (1998), Experimental Nephrology, 5, 429 (1997), Nippon Rinsho—Japanese Journal of Clinical Medicine, 54, 2583 (1996)], or a siRNA (short interfering RNA).

An antisense refers to one that allows nucleotide sequence-specific hybridization of a nucleic acid having a nucleotide sequence complementary to a certain target nucleic acid to enable the suppression of the expression of the target nucleic acid. As the nucleic acid used as the antisense, a DNA, an RNA or a nucleotide analogue, as well as a chimeric molecule thereof, or a derivative of the nucleic acid can also be used. Specifically, by preparing an antisense in accordance with a method described in Nature 432, 226 (2004) and the like, the expression can be suppressed. In addition, in the same way as with Anti-miR™ miRNA Inhibitors (manufactured by Ambion) and miRIDIAN miRNA Inhibitors (manufactured by GE Healthcare), the expression of a nucleic acid such as miRNA or miRNA precursor can be suppressed.

An siRNA refers to a short double-stranded RNA comprising the nucleotide sequence of a certain target nucleic acid, that is capable of suppressing the expression of the target nucleic acid by RNA interference (RNAi). The sequence of an siRNA can be designed as appropriate from the target nucleotide sequence on the basis of conditions shown in the literature [Genes Dev., 13, 3191 (1999)]. For example, by synthesizing two RNAs having a 19-nucleotide sequence selected and a complementary sequence, with TT added to the 3′ terminus of each thereof using a nucleic acid synthesizer, and performing annealing, an siRNA can be prepared. By inserting a DNA corresponding to the above-described 19-nucleotide sequence selected into an siRNA expression vector such as pSilencer 1.0-U6 (manufactured by Ambion) or pSUPER (manufactured by OligoEngine), a vector that expresses an siRNA capable of suppressing the expression of the gene can be prepared. Although any siRNA can be used to suppress a nucleic acid such as a miRNA used in the present invention, as far as it is capable of suppressing the activity of the nucleic acid, preference is given to siRNAs designed on the basis of continuous sequence information on the 9th nucleotide and beyond of each nucleotide sequence shown by any one of SEQ ID NO:1-939, 2057-2132, 2212-2261 and 2315-2321. The number of nucleotide residues constituting one strand of the siRNA is preferably 17 to 30 residues, more preferably 18 to 25 residues, still more preferably 19 to 23 residues.

Using an antisense or siRNA specific for a nucleic acid such as miRNA expressed in cancer cells, or a precursor of the miRNA, the expression of a miRNA expressed in cancer cells, a precursor of the miRNA and the like can be suppressed. For example, by administering an antisense DNA or siRNA specific for the miRNA or the miRNA precursor, the activation of the miRNA can be suppressed, and the action of the miRNA or miRNA precursor in cancer cells can be controlled.

In addition, in the case of a patient affected by an abnormality of the expression of a miRNA expressed in cancer cells or a precursor thereof, by administering an antisense oligonucleotide or siRNA specific for the miRNA or precursor thereof to the patient, it is possible to control a function of cancer cells to treat a disease that develops as a result of the above-described expression abnormality. Hence, an antisense oligonucleotide or siRNA that is specific for the miRNA or precursor thereof is useful as a therapeutic agent for a disease caused by a cell proliferation abnormality of cancer cells.

When an antisense oligonucleotide or siRNA specific for a nucleic acid such as a miRNA or a precursor thereof is used as the above-described therapeutic agent, the antisense oligonucleotide or siRNA alone, or a nucleic acid that encodes the same after being inserted into an appropriate vector such as a retrovirus vector, adenovirus vector, or adeno-associated virus vector, may be prepared as a pharmaceutical preparation according to the conventional method described in 9 below, and administered.

7. Methods of Suppressing Functions of Genes Using a Nucleic Acid Such as miRNA or miRNA Precursor

Any method can be used to suppress a function or the expression of the target gene for a nucleic acid, as far as it is based on an activity of a nucleic acid such as a miRNA to suppress the expression of the mRNA having the target nucleotide sequence. For example, a method can be mentioned wherein a nucleic acid such as a miRNA is expressed to increase the amount of the nucleic acid such as the miRNA in cells, whereby the translation of the mRNA having the target sequence is suppressed to suppress the expression of the gene. The expression of a nucleic acid can be achieved by the method described in 5 above. As examples of mRNAs having a target nucleotide sequence for a nucleic acid consisting of a nucleotide sequence shown by any one of SEQ ID NO:1-939, 2057-2132, 2212-2261 and 2315-2321, the respective sets of genes shown in the foregoing Table 4 can be mentioned.

Using an siRNA against a target gene shown in Table 4, it is possible to suppress a function of the target gene.

8. Methods of Screening for Substances that Promote or Suppress the Expression or a Function of a Nucleic Acid Such as a miRNA or miRNA Precursor Using the Nucleic Acid

A substance that promotes or suppresses the expression or a function of a nucleic acid such as a miRNA or precursor thereof can be screened for using a nucleic acid. For example, a nucleotide sequence to be targeted for screening is chosen from the nucleotide sequence of a nucleic acid, and by means of cells that express a nucleic acid having the nucleotide sequence, a substance that promotes or suppresses the expression or a function of the chosen miRNA or precursor thereof can be screened for.

As cells that express a miRNA or miRNA precursor and the like, used for screening, cancer cells, as well as transformant cells obtained by introducing a vector that expresses a nucleic acid having the nucleotide sequence into a host cell such as an animal cell or yeast, cells incorporating a nucleic acid having the nucleotide sequence introduced directly without using a vector and the like as described in 5 above can also be used.

As specific screening methods, a method comprising using a change in the expression level of a nucleic acid such as an miRNA or precursor thereof to be targeted in the screening as an index, as well as a method comprising using a change in the expression level of a mRNA having a target sequence for a nucleic acid such as a miRNA or a gene product encoded thereby as an index can be mentioned.

(a) Screening Method Comprising Using a Change in the Expression Level of a Nucleic Acid Such as a miRNA or Precursor Thereof to be Targeted in the Screening as an Index

A test substance is brought into contact with a cell that expresses a nucleic acid, and with a change in the expression level of the nucleic acid selected as an index, a substance that promotes or suppresses the nucleic acid of expression such as a miRNA and precursor thereof is obtained. The expression level of a nucleic acid can be detected by the method described in 3 above.

(b) Screening Method Comprising Using a Change in the Expression Level of a mRNA Having a Target Sequence for a Nucleic Acid Such as a miRNA to be Targeted in the Screening or a Gene Product Encoded Thereby as an Index

A test substance is brought into contact with a cell that expresses the mRNA, and with a change in the expression level of a mRNA having a target sequence of the nucleic acid selected or a gene product encoded thereby as an index, a substance that promotes or suppresses the expression or a function of a nucleic acid such as a miRNA and a precursor thereof is obtained. Alternatively, a DNA having a target sequence of a nucleic acid such as miRNA inserted into the 3′ UTR of an appropriate reporter gene expression vector is prepared and introduced into a host cell suitable for the expression vector, a test substance is brought into contact with the cell, and with a change in the expression level of the reporter gene as an index, a substance that promotes or suppresses the expression or a function of a nucleic acid such as a miRNA and a precursor thereof is obtained.

Choice of a target sequence can be achieved by the method described in 7 above; as examples of a mRNA having a target sequence of a nucleic acid such as a miRNA consisting of the nucleotide sequence of any one of SEQ ID NO:1 to 939, 2057 to 2132, 2212 to 2261 and 2315 to 2321, the above-described genes shown in Table 4 above can be exemplified.

9. Agent for Suppressing or Promoting Cell Proliferation Using a Nucleic Acid Such as miRNA or miRNA Precursor

Nucleic acids such as miRNAs and miRNA precursors, and nucleic acids having nucleotide sequences complementary to the nucleotide sequences thereof can be utilized as agents for suppressing or promoting cell proliferation because they control the expression of genes having target sequences.

As agents for suppressing cell proliferation, the nucleic acids (a) to (h) below can be mentioned.
(a) A nucleic acid consisting of a nucleotide sequence shown by any one of SEQ ID NO:1-3, 5-7, 11, 14, 15, 17, 24, 25, 29-33, 35, 37, 39-54, 56-58, 61-63, 66-68, 70, 72-82, 84, 85, 87-112, 114-121, 125, 128-167, 193-219, 263-278, 306-324, 357-368, 476-492, 522-557, 561-564, 572-576, 578-683, 693-714, 735-743, 746-751, 754-762, 765-803, 853-855, 888-891, 899-912, 914-926, 930-939, 2058, 2060, 2061, 2063-2065, 2067-2071, 2073-2078, 2080, 2082, 2084-2092, 2096-2108, 2110, 2111, 2113, 2117, 2120-2122, 2125-2128, 2132, 2212, 2213, 2216, 2219, 2220, 2227-2229, 2231-2237, 2240-2243, 2248-2253, 2255-2261, 2317 and 2318;
(b) a nucleic acid of 17 to 28 nucleotides comprising a nucleic acid consisting of a nucleotide sequence shown by any one of SEQ ID NO:1-3, 5-7, 11, 14, 15, 17, 24, 25, 29-33, 35, 37, 39-54, 56-58, 61-63, 66-68, 70, 72-82, 84, 85, 87-112, 114-121, 125, 128-167, 193-219, 263-278, 306-324, 357-368, 476-492, 522-557, 561-564, 572-576, 578-683, 693-714, 735-743, 746-751, 754-762, 765-803, 853-855, 888-891, 899-912, 914-926, 930-939, 2058, 2060, 2061, 2063-2065, 2067-2071, 2073-2078, 2080, 2082, 2084-2092, 2096-2108, 2110, 2111, 2113, 2117, 2120-2122, 2125-2128, 2132, 2212, 2213, 2216, 2219, 2220, 2227-2229, 2231-2237, 2240-2243, 2248-2253, 2255-2261, 2317 and 2318;
(c) a nucleic acid consisting of a nucleotide sequence having an identity of 90% or more to a nucleotide sequence shown by any one of SEQ ID NO:l-3, 5-7, 11, 14, 15, 17, 24, 25, 29-33, 35, 37, 39-54, 56-58, 61-63, 66-68, 70, 72-82, 84, 85, 87-112, 114-121, 125, 128-167, 193-219, 263-278, 306-324, 357-368, 476-492, 522-557, 561-564, 572-576, 578-683, 693-714, 735-743, 746-751, 754-762, 765-803, 853-855, 888-891, 899-912, 914-926, 930-939, 2058, 2060, 2061, 2063-2065, 2067-2071, 2073-2078, 2080, 2082, 2084-2092, 2096-2108, 2110, 2111, 2113, 2117, 2120-2122, 2125-2128, 2132, 2212, 2213, 2216, 2219, 2220, 2227-2229, 2231-2237, 2240-2243, 2248-2253, 2255-2261, 2317 and 2318;
(d) a nucleic acid that hybridizes under stringent conditions with a complementary strand for a nucleic acid consisting of a nucleotide sequence shown by any one of SEQ ID NO:1-3, 5-7, 11, 14, 15, 17, 24, 25, 29-33, 35, 37, 39-54, 56-58, 61-63, 66-68, 70, 72-82, 84, 85, 87-112, 114-121, 125, 128-167, 193-219, 263-278, 306-324, 357-368, 476-492, 522-557, 561-564, 572-576, 578-683, 693-714, 735-743, 746-751, 754-762, 765-803, 853-855, 888-891, 899-912, 914-926, 930-939, 2058, 2060, 2061, 2063-2065, 2067-2071, 2073-2078, 2080, 2082, 2084-2092, 2096-2108, 2110, 2111, 2113, 2117, 2120-2122, 2125-2128, 2132, 2212, 2213, 2216, 2219, 2220, 2227-2229, 2231-2237, 2240-2243, 2248-2253, 2255-2261, 2317 and 2318;
(e) a nucleic acid comprising the sequence of 2nd to 8th nucleotides of a nucleotide sequence shown by any one of SEQ ID NO:1-3, 5-7, 11, 14, 15, 17, 24, 25, 29-33, 35, 37, 39-54, 56-58, 61-63, 66-68, 70, 72-82, 84, 85, 87-112, 114-121, 125, 128-167, 193-219, 263-278, 306-324, 357-368, 476-492, 522-557, 561-564, 572-576, 578-683, 693-714, 735-743, 746-751, 754-762, 765-803, 853-855, 888-891, 899-912, 914-926, 930-939, 2058, 2060, 2061, 2063-2065, 2067-2071, 2073-2078, 2080, 2082, 2084-2092, 2096-2108, 2110, 2111, 2113, 2117, 2120-2122, 2125-2128, 2132, 2212, 2213, 2216, 2219, 2220, 2227-2229, 2231-2237, 2240-2243, 2248-2253, 2255-2261, 2317 and 2318;
(f) a nucleic acid consisting of a nucleotide sequence shown by any one of SEQ ID NO:940-945, 947, 948, 952, 953, 956-958, 960, 968, 969, 973-978, 980, 982, 984-1001, 1003-1005, 1008-1009, 1012-1014, 1016, 1018-1029, 1031, 1033-1059, 1064-1069, 1073, 1076-1133, 1163-1188, 1232-1259, 1288-1309, 1343-1354, 1479-1495, 1529-1566, 1572-1576, 1585-1589, 1591-1709, 1719-1744, 1765-1777, 1780-1788, 1791-1829, 1898-1900, 1998-2001, 2009-2050, 2052-2056, 2134, 2136, 2137, 2139-2143, 2145-2151, 2153-2158, 2160, 2162, 2164-2172, 2176-2188, 2190, 2191, 2193, 2197, 2200-2202, 2205-2207, 2211, 2262-2265, 2268, 2271-2273, 2281-2283, 2285-2292, 2295-2297, 2302-2306, 2308-2314, 2324 and 2325;
(g) a nucleic acid consisting of a nucleotide sequence having an identity of 90% or more to a nucleotide sequence shown by any one of SEQ ID NO:940-945, 947, 948, 952, 953, 956-958, 960, 968, 969, 973-978, 980, 982, 984-1001, 1003-1005, 1008-1009, 1012-1014, 1016, 1018-1029, 1031, 1033-1059, 1064-1069, 1073, 1076-1133, 1163-1188, 1232-1259, 1288-1309, 1343-1354, 1479-1495, 1529-1566, 1572-1576, 1585-1589, 1591-1709, 1719-1744, 1765-1777, 1780-1788, 1791-1829, 1898-1900, 1998-2001, 2009-2050, 2052-2056, 2134, 2136, 2137, 2139-2143, 2145-2151, 2153-2158, 2160, 2162, 2164-2172, 2176-2188, 2190, 2191, 2193, 2197, 2200-2202, 2205-2207, 2211, 2262-2265, 2268, 2271-2273, 2281-2283, 2285-2292, 2295-2297, 2302-2306, 2308-2314, 2324 and 2325; and
(h) a nucleic acid that hybridizes under stringent conditions with a complementary strand for a nucleic acid consisting of a nucleotide sequence shown by any one of SEQ ID NO:940-945, 947, 948, 952, 953, 956-958, 960, 968, 969, 973-978, 980, 982, 984-1001, 1003-1005, 1008-1009, 1012-1014, 1016, 1018-1029, 1031, 1033-1059, 1064-1069, 1073, 1076-1133, 1163-1188, 1232-1259, 1288-1309, 1343-1354, 1479-1495, 1529-1566, 1572-1576, 1585-1589, 1591-1709, 1719-1744, 1765-1777, 1780-1788, 1791-1829, 1898-1900, 1998-2001, 2009-2050, 2052-2056, 2134, 2136, 2137, 2139-2143, 2145-2151, 2153-2158, 2160, 2162, 2164-2172, 2176-2188, 2190, 2191, 2193, 2197, 2200-2202, 2205-2207, 2211, 2262-2265, 2268, 2271-2273, 2281-2283, 2285-2292, 2295-2297, 2302-2306, 2308-2314, 2324 and 2325.

As agents for promoting cell proliferation, the following nucleic acids (i)-(p) can be mentioned.
(i) A nucleic acid consisting of a nucleotide sequence shown by any one of SEQ ID NO:4, 8-10, 12, 13, 16, 18-23, 26-28, 34, 36, 38, 55, 59, 60, 64, 65, 69, 71, 83, 86, 113, 122-124, 126, 127, 168-192, 220-262, 279-305, 325-356, 369-475, 493-521, 558-560, 565-571, 577, 684-692, 715-734, 744, 745, 752, 753, 763, 764, 804-852, 856-887, 892-898, 913, 927-929, 2057, 2059, 2062, 2066, 2072, 2079, 2081, 2083, 2093-2095, 2109, 2112, 2114-2116, 2118, 2119, 2123, 2124, 2129-2131, 2214, 2215, 2218, 2221-2226, 2230, 2238, 2239, 2244-2247, 2254, 2315, 2316 and 2319-2321;
(j) a nucleic acid of 17-28 nucleotides comprising a nucleic acid consisting of a nucleotide sequence shown by any one of SEQ ID NO:4, 8-10, 12, 13, 16, 18-23, 26-28, 34, 36, 38, 55, 59, 60, 64, 65, 69, 71, 83, 86, 113, 122-124, 126, 127, 168-192, 220-262, 279-305, 325-356, 369-475, 493-521, 558-560, 565-571, 577, 684-692, 715-734, 744, 745, 752, 753, 763, 764, 804-852, 856-887, 892-898, 913, 927-929, 2057, 2059, 2062, 2066, 2072, 2079, 2081, 2083, 2093-2095, 2109, 2112, 2114-2116, 2118, 2119, 2123, 2124, 2129-2131, 2214, 2215, 2218, 2221-2226, 2230, 2238, 2239, 2244-2247, 2254, 2315, 2316 and 2319-2321;
(k) a nucleic acid consisting of a nucleotide sequence having an identity of 90% or more to a nucleotide sequence shown by any one of SEQ ID NO:4, 8-10, 12, 13, 16, 18-23, 26-28, 34, 36, 38, 55, 59, 60, 64, 65, 69, 71, 83, 86, 113, 122-124, 126, 127, 168-192, 220-262, 279-305, 325-356, 369-475, 493-521, 558-560, 565-571, 577, 684-692, 715-734, 744, 745, 752, 753, 763, 764, 804-852, 856-887, 892-898, 913, 927-929, 2057, 2059, 2062, 2066, 2072, 2079, 2081, 2083, 2093-2095, 2109, 2112, 2114-2116, 2118, 2119, 2123, 2124, 2129-2131, 2214, 2215, 2218, 2221-2226, 2230, 2238, 2239, 2244-2247, 2254, 2315, 2316 and 2319-2321;
(l) a nucleic acid that hybridizes under stringent conditions with a complementary strand for a nucleic acid consisting of a nucleotide sequence shown by any one of SEQ ID NO:4, 8-10, 12, 13, 16, 18-23, 26-28, 34, 36, 38, 55, 59, 60, 64, 65, 69, 71, 83, 86, 113, 122-124, 126, 127, 168-192, 220-262, 279-305, 325-356, 369-475, 493-521, 558-560, 565-571, 577, 684-692, 715-734, 744, 745, 752, 753, 763, 764, 804-852, 856-887, 892-898, 913, 927-929, 2057, 2059, 2062, 2066, 2072, 2079, 2081, 2083, 2093-2095, 2109, 2112, 2114-2116, 2118, 2119, 2123, 2124, 2129-2131, 2214, 2215, 2218, 2221-2226, 2230, 2238, 2239, 2244-2247, 2254, 2315, 2316 and 2319-2321;
(m) a nucleic acid comprising the sequence of 2nd to 8th nucleotides of a nucleotide sequence shown by any one of SEQ ID NO:4, 8-10, 12, 13, 16, 18-23, 26-28, 34, 36, 38, 55, 59, 60, 64, 65, 69, 71, 83, 86, 113, 122-124, 126, 127, 168-192, 220-262, 279-305, 325-356, 369-475, 493-521, 558-560, 565-571, 577, 684-692, 715-734, 744, 745, 752, 753, 763, 764, 804-852, 856-887, 892-898, 913, 927-929, 2057, 2059, 2062, 2066, 2072, 2079, 2081, 2083, 2093-2095, 2109, 2112, 2114-2116, 2118, 2119, 2123, 2124, 2129-2131, 2214, 2215, 2218, 2221-2226, 2230, 2238, 2239, 2244-2247, 2254, 2315, 2316 and 2319-2321;
(n) a nucleic acid consisting of a nucleotide sequence shown by any one of SEQ ID NO:946, 949-951, 954, 955, 959, 961-967, 970-972, 979, 981, 983, 1002, 1006, 1007, 1010, 1011, 1015, 1017, 1030, 1032, 1060-1063, 1070-1072, 1074, 1075, 1134-1162, 1189-1231, 1260-1287, 1310-1342, 1355-1478, 1496-1528, 1567-1571, 1577-1584, 1590, 1710-1718, 1745-1764, 1778, 1779, 1789, 1790, 1830-1897, 1901-1997, 2002-2008, 2051, 2133, 2135, 2138, 2144, 2152, 2159, 2161, 2163, 2173-2175, 2189, 2192, 2194-2196, 2198, 2199, 2203, 2204, 2208-2210, 2266, 2267, 2270, 2274-2280, 2284, 2293, 2294, 2298-2301, 2307, 2322, 2323 and 2326-2328;
(o) a nucleic acid consisting of a nucleotide sequence having an identity of 90% or more to a nucleotide sequence shown by any one of SEQ ID NO:946, 949-951, 954, 955, 959, 961-967, 970-972, 979, 981, 983, 1002, 1006, 1007, 1010, 1011, 1015, 1017, 1030, 1032, 1060-1063, 1070-1072, 1074, 1075, 1134-1162, 1189-1231, 1260-1287, 1310-1342, 1355-1478, 1496-1528, 1567-1571, 1577-1584, 1590, 1710-1718, 1745-1764, 1778, 1779, 1789, 1790, 1830-1897, 1901-1997, 2002-2008, 2051, 2133, 2135, 2138, 2144, 2152, 2159, 2161, 2163, 2173-2175, 2189, 2192, 2194-2196, 2198, 2199, 2203, 2204, 2208-2210, 2266, 2267, 2270, 2274-2280, 2284, 2293, 2294, 2298-2301, 2307, 2322, 2323 and 2326-2328; and
(p) a nucleic acid that hybridizes under stringent conditions with a complementary strand for a nucleic acid consisting of a nucleotide sequence shown by any one of SEQ ID NO:946, 949-951, 954, 955, 959, 961-967, 970-972, 979, 981, 983, 1002, 1006, 1007, 1010, 1011, 1015, 1017, 1030, 1032, 1060-1063, 1070-1072, 1074, 1075, 1134-1162, 1189-1231, 1260-1287, 1310-1342, 1355-1478, 1496-1528, 1567-1571, 1577-1584, 1590, 1710-1718, 1745-1764, 1778, 1779, 1789, 1790, 1830-1897, 1901-1997, 2002-2008, 2051, 2133, 2135, 2138, 2144, 2152, 2159, 2161, 2163, 2173-2175, 2189, 2192, 2194-2196, 2198, 2199, 2203, 2204, 2208-2210, 2266, 2267, 2270, 2274-2280, 2284, 2293, 2294, 2298-2301, 2307, 2322, 2323 and 2326-2328.

The above-described nucleic acids are suitably miRNAs or miRNA precursors.

As agents for promoting cell proliferation, nucleic acids consisting of a nucleotide sequence complementary to the nucleotide sequence of one of the nucleic acids (a) to (h) above can be mentioned; as agents for suppressing cell proliferation, nucleic acids consisting of a nucleotide sequence complementary to the nucleotide sequence of one of the nucleic acids (i) to (p) above can be mentioned. Vectors that express the above-described nucleic acids and nucleic acids that are complementary thereto can also be used as agents for suppressing or promoting cell proliferation.

Meanwhile, substances that suppress the expression of target genes for nucleic acids such as the above-described miRNAs, and substances that promote the expression of the target genes can also be used as agents for suppressing or promoting cell proliferation. As these substances, nucleic acids or vectors that express the same can also be used. As substances that suppress the expression of a target gene, an siRNA against the mRNA of the target gene and an antisense against the target gene can be mentioned; as substances that promote the expression of the target gene, an siRNA against a miRNA specific for the target gene and an antisense against the target gene can be mentioned.

Regarding the preparation forms, methods of administration and the like for an agent for suppressing or promoting cell proliferation, the same applies as with the diagnostic reagent and therapeutic drug containing a nucleic acid such as a miRNA or miRNA precursor described in 10 below.

10. Diagnostic Reagent and Therapeutic Drug Containing a Nucleic Acid Such as a miRNA or miRNA Precursor

Nucleic acids such as miRNAs and miRNA precursors can be utilized as drugs for treating diseases resulting from a cell proliferation abnormality and the like, such as cancers, because they control the expression of genes having a target sequence, or control the expression of nucleic acids such as miRNAs. Furthermore, siRNAs against the target gene for the nucleic acid can be utilized as drugs for treating diseases resulting from a cell proliferation or differentiation abnormality and the like because they control the expression of the gene. As diseases resulting from a cell proliferation abnormality and the like, cancers, arteriosclerosis, rheumatoid arthritis, prostatic hyperplasia, blood vessel restenosis after percutaneous transvascular coronary angioplasty, fibroid lung, glomerulonephritis, autoimmune diseases and the like can be mentioned.

By quantifying a nucleic acid or detecting a mutation therein, a disease resulting from a cell proliferation or differentiation abnormality and the like, such as a cancer, can be diagnosed.

A diagnostic reagent comprising a nucleic acid, according to the desired diagnostic method, may comprise reagents necessary for quantitation or detection of mutation of a nucleic acid, for example, buffering agents, salts, reaction enzymes, labeled proteins that bind to a nucleic acid, and a color developer for detection and the like.

Although a therapeutic agent containing a nucleic acid as an active ingredient can be administered alone, the same is normally desirably administered as a pharmaceutical preparation produced by an optionally chosen method known well in the technical field of pharmaceutical making with one or more pharmacologically acceptable carriers blended therein.

The route of administration used is desirably the most effective one in treatment; oral administration, or parenteral administration such as intraoral administration, airway administration, intrarectal administration, subcutaneous administration, intramuscular administration and intravenous administration can be mentioned, and desirably intravenous administration can be mentioned.

As dosage forms, sprays, capsules, tablets, granules, syrups, emulsions, suppositories, injection formulations, ointments, tapes and the like can be mentioned. As preparations appropriate for oral administration, emulsions, syrups, capsules, tablets, powders, granules and the like can be mentioned.

Liquid preparations like emulsions and syrups can be produced using water, saccharides such as sucrose, sorbitol, and fructose, glycols such as polyethylene glycol and propylene glycol, oils such as sesame oil, olive oil, and soybean oil, antiseptics such as p-hydroxybenzoic acid esters, flavors such as strawberry flavor and peppermint and the like as additives.

Capsules, tablets, powders, granules and the like can be produced using excipients such as lactose, glucose, sucrose, and mannitol, disintegrants such as starch and sodium alginate, lubricants such as magnesium stearate and talc, binders such as polyvinyl alcohol, hydroxypropylcellulose, and gelatin, surfactants such as fatty acid esters, plasticizers such as glycerin and the like as additives.

As appropriate preparations for parenteral administration, injection formulations, suppositories, sprays and the like can be mentioned.

An injection formulation is prepared using a carrier consisting of a salt solution, a glucose solution or a mixture of both and the like. A suppository is prepared using a carrier such as cacao butter, hydrogenated fat or carboxylic acid. A spray is prepared using a carrier that does not stimulate the recipient's oral cavity and airway mucosa, and that disperses the active ingredients as fine particles to facilitate the absorption thereof, and the like.

As examples of the carrier, specifically, lactose, glycerin and the like can be exemplified. Depending on the nature of the nucleic acid or miRNA precursor, and of the carrier used, preparations such as aerosols and dry powders are possible. In these parenteral preparations, components exemplified as additives for oral preparations can also be added.

The dose or frequency of administration varies depending on desired therapeutic effect, method of administration, duration of treatment, age, body weight and the like, and is normally 10 μg/kg to 20 mg/kg per day for an adult.

A therapeutic drug comprising a nucleic acid as an active ingredient can also be produced by blending a vector that expresses a nucleic acid and a nucleotide used in a nucleic acid therapeutic drug [Nature Genet., 8, 42(1994)].

The base used in the therapeutic drug may be any base for ordinary use in injection formulations; distilled water, solutions of salts such as sodium chloride or a mixture of sodium chloride and an inorganic salt, solutions of mannitol, lactose, dextran, glucose and the like, solutions of amino acids such as glycine and arginine, mixed solutions of organic acid solutions or salt solutions and glucose solution and the like can be mentioned. In accordance with a conventional method, using auxiliary agents such as an osmoregulator, a pH adjuster, a vegetable oil such as sesame oil or soybean oil, lecithin, and a surfactant such as a non-ionic surfactant in these bases, an injection formulation may be prepared as a solution, suspension, or dispersion. These injection formulations can also be prepared as preparations for dissolution before use, by procedures such as powdering and lyophilization. A therapeutic agent can be used for treatment as is in the case of a liquid, or after being dissolved in a base described above, optionally sterilized, in the case of a solid, just before treatment.

As a vector expressing a nucleic acid, the recombinant viral vector prepared in the method of the above-mentioned 5 can be mentioned, more specifically, retrovirus vector and lentivirus vector and the like can be mentioned.

For example, by combining a nucleic acid with a polylysine-conjugated antibody that is specific for adenovirus hexon protein to prepare a complex, and binding the complex obtained to an adenovirus vector, a viral vector can be prepared. The viral vector is capable of stably reaching the desired cell, being incorporated into cells by endosome, being decomposed in the cells, and efficiently expressing the nucleic acid.

A viral vector based on Sendai virus, which is a (−) strand RNA virus, has been developed (WO97/16538, WO97/16539), a Sendai virus incorporating a nucleic acid can be prepared using the Sendai virus.

A nucleic acid can also be transferred by a non-viral nucleic acid transfer method. The same can be transferred by, for example, calcium phosphate co-precipitation [Virology, 52, 456-467 (1973); Science, 209, 1414-1422 (1980)], microinjection method [Proc. Natl. Acad. Sci. USA, 77, 5399-5403 (1980); Proc. Natl. Acad. Sci. USA, 77, 7380-7384 (1980); Cell, 27, 223-231 (1981); Nature, 294, 92-94 (1981)], membrane fusion-mediated transfer mediated by liposome [Proc. Natl. Acad. Sci. USA, 84, 7413-7417 (1987); Biochemistry, 28, 9508-9514 (1989); J. Biol. Chem., 264, 12126-12129 (1989); Hum. Gene Ther., 3, 267-275, (1992); Science, 249, 1285-1288 (1990); Circulation, 83, 2007-2011 (1992)] or direct DNA uptake and receptor-mediated DNA transfer method [Science, 247, 1465-1468 (1990); J. Biol. Chem., 266, 14338-14342 (1991); Proc. Natl. Acad. Sci. USA, 87, 3655-3659 (1991); J. Biol. Chem., 264, 16985-16987 (1989); BioTechniques, 11, 474-485 (1991); Proc. Natl. Acad. Sci. USA, 87, 3410-3414 (1990); Proc. Natl. Acad. Sci. USA, 88, 4255-4259 (1991); Proc. Natl. Acad. Sci. USA, 87, 4033-4037 (1990); Proc. Natl. Acad. Sci. USA, 88, 8850-8854 (1991); Hum. Gene Ther., 3, 147-154 (1991)] and the like.

Membrane fusion-mediated transfer mediated by liposome allows a nucleic acid to be incorporated locally in the tissue, and to be expressed, by administering a liposome preparation directly to the target tissue [Hum. Gene Ther., 3, 399 (1992)]. For direct targeting of a DNA to a focus, direct DNA uptake technology is preferable.

For receptor-mediated DNA transfer, for example, a method performed by binding a DNA (usually assuming the form of a covalently cyclized supercoiled plasmid) to a protein ligand via polylysine can be mentioned. A ligand is chosen on the basis of the presence of a corresponding ligand receptor on the cell surface of the desired cell or tissue. The ligand-DNA conjugate can be injected directly into a blood vessel as desired, and can be directed to a target tissue wherein receptor binding and DNA-protein complex internalization occur. To prevent the destruction of the DNA in a cell, an adenovirus may be infected simultaneously to destroy the endosome function.

### 11. Method of Measuring Proliferation of Cancer Cells and Other Cells

The method of measuring cell proliferation is not particularly limited, as far as it enables a measurement of an index that reflects cell count or cell proliferation rate. Viable cell counting, DNA synthesis rate measurements, total protein content measurements and the like can be used. As a method of evaluating viable cell counts, a method wherein the ATP content in cells is measured can be mentioned. It is known that the ATP content in cells is proportional to the number of cells in culture (J. Immunol. Meth. 160, 81-88 (1993)). As more specific methods of measuring the ATP content in cells, the MTT method, the XTT method and the like can be mentioned (J. Immunol. Meth. 65, 55-63 (1983)). A method can also be mentioned wherein ATP is measured by luminescence of a luciferin substrate by the ATP-dependent enzyme luciferase. As a kit for measuring the ATP content in cells, for example, CellTite-Glo® Luminescent Cell viability Assay (manufactured by Promega) and the like may be used.

### 12. Methods of Measuring the Degree of Cell Death of Cancer Cells

As methods of measuring the degree of cell death, a method wherein dead cells are stained with a dye such as Propium Iodide, a method wherein the activity of an enzyme leaked extracellularly as a result of cell death is measured, and the like can be used. For the latter, for example, a method wherein the enzyme activity of extracellularly leaked adenylate kinase is measured can be utilized. More specifically, ToxiLight(R) Non-Destructive Cytotoxicity BioAssay Kit (manufactured by Lonza) and the like may be used.

It is also possible to measure the degree of apoptosis (programmed cell death), which is important in relation to cancers, out of the various types of cell death. As methods of evaluating cell apoptosis, a method wherein the degree of DNA fragmentation is measured, a method wherein changes in cell membrane constituent lipids are measured, and a method wherein the activity of caspase 3/7, an intracellular protease induced upon apoptosis, is measured, can be mentioned. As a more specific method of measuring caspase 3/7 activity in cells, a method wherein a luciferin substrate liberated by caspase 3/7 activity is measured by luciferin luminescence by a luciferase enzyme reaction can be mentioned. As kits for measuring caspase 3/7 activity in cells, for example, Caspase-Glo® 3/7 Assay (manufactured by Promega) and the like may be used.

The present invention is hereinafter described specifically by means of the following Examples. However, the present invention is not limited to these Examples.

### Example 1

Cell Viability and Apoptotic Activity in Colorectal Cancer-Derived Cell Line with a miRNA Expressed Forcedly Therein

Pre-miR™ miRNA Precursor Molecules (manufactured by Ambion) were introduced into a colorectal cancer-derived cell line, and the influences of the miRNAs produced from the molecules on cell viability and apoptotic activity were examined.

The DLD-1 human colorectal cancer-derived cell line (hereinafter referred to as DLD-1) was obtained from the American Type Culture Collection (hereinafter referred to as ATCC). Cells were cultured with an RPMI1640 medium (manufactured by Invitrogen) containing 10% fetal bovine serum (FBS) (manufactured by JRH Biosciences) in a 37° C. 5% CO₂ concentration incubator.

DLD-1 was sown to a 96-well plate at 2500 cells per well, and cultured in an RPMI medium containing 10% FBS overnight. One day later, Pre-miR™miRNA Precursor Molecules that produce hsa-miR-7 (SEQ ID NO:3), 15a, 15b, 16, 18a (SEQ ID NO:5), 18a* (SEQ ID NO:7), 28 (SEQ ID NO:17), 95 (SEQ ID NO:24), 124a, 132 (SEQ ID NO:29), 133a (SEQ ID NO:31), 133b (SEQ ID NO:32), 147 (SEQ ID NO:37), 181b, 181c (SEQ ID NO:39), 181d (SEQ ID NO:40), 184 (SEQ ID NO:43), 192 (SEQ ID NO:44), 193a (SEQ ID NO:46), 193b (SEQ ID NO:47), 195 (SEQ ID NO:48), 196a (SEQ ID NO:51), 196b (SEQ ID NO:52), 197 (SEQ ID NO:53), 202 (SEQ ID NO:56), 204 (SEQ ID NO:57), 211 (SEQ ID NO:58), 212 (SEQ ID NO:30), 215 (SEQ ID NO:45), 299-3p (SEQ ID NO:62), 299-5p (SEQ ID NO:63), 324-3p (SEQ ID NO:66), 337 (SEQ ID NO:70), 342 (SEQ ID NO:72), 362 (SEQ ID NO:74), 368 (SEQ ID NO:75), 376a (SEQ ID NO:77), 376b (SEQ ID NO:78), 380-5p (SEQ ID NO:81), 422a (SEQ ID NO:84), 422b (SEQ ID NO:85), 424 (SEQ ID NO:49), 432* (SEQ ID NO:88), 452 (SEQ ID NO:92), 452* (SEQ ID NO:93), 455 (SEQ ID NO:94), 483 (SEQ ID NO:95), 489 (SEQ ID NO:98), 491 (SEQ ID NO:99), 493-5p (SEQ ID NO:100), 494 (SEQ ID NO:101), 497 (SEQ ID NO:50), 501 (SEQ ID NO:102), 504 (SEQ ID NO:103), 506 (SEQ ID NO:105), 512-3p (SEQ ID NO:110), 512-5p (SEQ ID NO:111), 517* (SEQ ID NO:114), 517b (SEQ ID NO:117), 517c (SEQ ID NO:116), 518b (SEQ ID NO:118), 518c* (SEQ ID NO:121), 518d (SEQ ID NO:120), 523 (SEQ ID NO:125), 527 (SEQ ID NO:129), let-7g, and let-7i were introduced into the DLD-1 to obtain a final concentration of 5 nM or 25 nM by a lipofection method, specifically a method using Lipofectamine2000 (manufactured by Invitrogen). Pre-miR™miRNA Precursor Molecules-Negative Control #2 (hereinafter, referred to as miR-negacon#2) (manufactured by Ambion) was also introduced into DLD-1, and this was used as the negative control. The lipofection was performed per the directions attached to the product.

Three days after introduction of the molecules by a lipofection method, cell viability was measured using CellTiter-Glo™luminescent Cell Viability Assay (manufactured by Promega) according to the method described in the protocol attached to the product. Taking the cell viability of DLD-1 in a control plot (Lipofectamine2000 only) as 1.0, the relative cell viability of each was calculated. As a result, as shown in Table 5, with introduction of molecules that produce hsa-miR-7, 28, 95, 147, 181d, 192, 193a, 193b, 195, 196a, 197, 211, 215, 299-3p, 299-5p, 337, 342, 362, 368, 376a, 376b, 380-5p, 422b, 432*, 452, 483, 489, 491, 493-5p, 494, 501, 506, 517c, 518b, and 518d, decreases of 40% or more in cell viability were observed. With introduction of molecules that produce hsa-miR-15b and 124a, which are known to be associated with cancers, decreases of 40% or more in cell viability were observed.

TABLE 5

| Introduced miRNA | cell viability (25 nM) | cell viability (5 nM) |
|---|---|---|
| miR-362 | 0.31 | 0.83 |
| miR-197 | 0.31 | 0.39 |
| miR-342 | 0.32 | 0.67 |
| miR-299-3p | 0.33 | 0.51 |
| miR-376b | 0.34 | 0.62 |
| miR-192 | 0.35 | 0.62 |
| miR-147 | 0.37 | 0.53 |
| miR-452 | 0.39 | 0.54 |
| miR-491 | 0.40 | 0.71 |
| miR-124a | 0.44 | 0.70 |
| miR-493-5p | 0.44 | 0.74 |
| miR-215 | 0.44 | 0.47 |
| miR-483 | 0.47 | 0.73 |
| miR-432* | 0.47 | 0.68 |
| miR-95 | 0.47 | 0.59 |
| miR-376a | 0.48 | 0.71 |
| miR-506 | 0.50 | 0.60 |
| miR-337 | 0.50 | 0.78 |
| miR-195 | 0.51 | 0.79 |
| miR-181d | 0.51 | 0.88 |
| miR-7 | 0.51 | 0.62 |
| miR-368 | 0.51 | 0.73 |
| miR-489 | 0.52 | 0.74 |
| miR-494 | 0.53 | 0.80 |
| miR-28 | 0.53 | 0.67 |
| miR-518d | 0.54 | 0.57 |
| miR-422b | 0.55 | 0.76 |
| miR-517c | 0.57 | 0.69 |
| miR-193a | 0.57 | 0.78 |
| miR-193b | 0.57 | 0.76 |
| miR-299-5p | 0.57 | 0.75 |
| miR-15b | 0.58 | 0.77 |
| miR-501 | 0.59 | 0.70 |
| miR-211 | 0.59 | 0.73 |
| miR-196a | 0.60 | 0.82 |
| miR-380-5p | 0.60 | 0.65 |
| miR-518b | 0.61 | 0.60 |
| miR-negacon#2 | 0.96 | 1.00 |

Two days after introduction of the molecules (final concentration 25 nM) by a lipofection method, caspase 3/7 activity was measured using Caspase-Glo^{R} 3/7 assay (manufactured by Promega) per the directions described in the protocol attached to the product. Taking the caspase 3/7 activity value of DLD-1 in a control plot (Lipofectamine2000 only) as 1.0, the relative caspase 3/7 activity value of each was calculated. As a result, as shown in Table 6, with introduction of molecules that produce hsa-miR-18a, 18a*, 132, 133a, 133b, 181c, 181d, 184, 192, 193a, 195, 196a, 196b, 197, 202, 204, 211, 212, 215, 299-3p, 299-5p, 324-3p, 337, 342, 362, 368, 376a, 380-5p, 422a, 422b, 424, 432*, 452*, 455, 483, 491, 493-5p, 497, 501, 504, 512-3p, 512-5p, 517*, 517b, 517c, 518c*, 523, and 527, increases of 50% or more in caspase 3/7 activity were observed. With introduction of molecules that produce hsa-miR-15a, 15b, 16, 181b, let-7g, and let-7i, which are known to be associated with cancers, increases of 50% or more in caspase 3/7 activity were observed.

TABLE 6

| introduced miRNA | caspase 3/7 activity |
|---|---|
| miR-337 | 3.90 |
| miR-452* | 3.61 |
| miR-491 | 3.33 |
| miR-133a | 3.26 |
| miR-299-3p | 3.19 |
| miR-422b | 2.94 |
| miR-422a | 2.65 |
| miR-512-3p | 2.57 |
| miR-299-5p | 2.50 |
| miR-368 | 2.44 |
| miR-432* | 2.43 |
| miR-197 | 2.39 |
| miR-195 | 2.30 |
| miR-133b | 2.25 |
| miR-527 | 2.12 |
| miR-380-5p | 2.07 |
| miR-181b | 2.06 |
| miR-15b | 2.04 |
| miR-132 | 2.01 |
| miR-455 | 1.97 |
| miR-192 | 1.97 |
| miR-501 | 1.93 |
| miR-181d | 1.93 |
| miR-193a | 1.93 |
| miR-181c | 1.93 |
| miR-512-5p | 1.90 |
| miR-215 | 1.89 |
| miR-362 | 1.89 |
| miR-15a | 1.83 |
| miR-493-5p | 1.83 |
| miR-517* | 1.81 |
| miR-324-3p | 1.80 |
| let-7g | 1.78 |
| miR-517b | 1.77 |
| miR-342 | 1.71 |
| miR-504 | 1.69 |
| miR-196a | 1.69 |
| miR-518c* | 1.67 |
| let-7i | 1.66 |
| miR-196b | 1.65 |
| miR-212 | 1.65 |
| miR-424 | 1.64 |
| miR-204 | 1.64 |
| miR-483 | 1.63 |
| miR-202 | 1.60 |
| miR-523 | 1.59 |
| miR-497 | 1.59 |
| miR-517c | 1.58 |
| miR-376a | 1.58 |
| miR-184 | 1.56 |
| miR-211 | 1.52 |
| miR-16 | 1.51 |
| miR-18a | 1.51 |
| miR-18a* | 1.51 |
| miR-negacon#2 | 0.96 |

In another test, DLD-1 was sown to a 96-well plate at 2500 cells per well, and cultured in an RPMI medium containing 10% FBS overnight. One day later, Pre-miR™miRNA Precursor Molecules (manufactured by Ambion) that produce hsa-miR-7 (SEQ ID NO:3), 24 (SEQ ID NO:11), 28 (SEQ ID NO:17), 34c, 95 (SEQ ID NO:24), 124a, 132 (SEQ ID NO:29), 134 (SEQ ID NO:33), 142-3p (SEQ ID NO:35), 147 (SEQ ID NO:37), 181a, 182* (SEQ ID NO:41), 183 (SEQ ID NO:42), 192 (SEQ ID NO:44), 197 (SEQ ID NO:53), 211 (SEQ ID NO:58), 222 (SEQ ID NO:61), 299-3p (SEQ ID NO:62), 329 (SEQ ID NO:68), 337 (SEQ ID NO:70), 342 (SEQ ID NO:72), 362 (SEQ ID NO:74), 368 (SEQ ID NO:75), 376a (SEQ ID NO:77), 376b (SEQ ID NO:78), 377 (SEQ ID NO:79), 380-5p (SEQ ID NO:81), 422b (SEQ ID NO:85), 424 (SEQ ID NO:49), 431 (SEQ ID NO:87), 432* (SEQ ID NO:88), 452 (SEQ ID NO:92), 452* (SEQ ID NO:93), 455 (SEQ ID NO:94), 483 (SEQ ID NO:95), 485-5p (SEQ ID NO:96), 489 (SEQ ID NO:98), 491 (SEQ ID NO:99), 493-5p (SEQ ID NO:100), 494 (SEQ ID NO:101), 505 (SEQ ID NO:104), 506 (SEQ ID NO:105), 509 (SEQ ID NO:107), 510 (SEQ ID NO:108), 517a (SEQ ID NO:115), and 517c (SEQ ID NO:116) were introduced into the DLD-1 to obtain a final concentration of 50 nM by a lipofection method, specifically a method using Lipofectamine2000 (manufactured by Invitrogen). miR-negacon#2 (manufactured by Ambion) was also introduced into DLD-1, and this was used as the negative control. The lipofection was performed per the directions attached to the product.

Three or five days after introduction of the molecules by the lipofection method, cell viability was measured using CellTiter-Glo™Luminescent Cell Viability Assay (manufactured by Promega) per the directions attached to the product. Taking the cell viability for the DLD-1 in the miR-negacon#2 introduction plot as 1.0, respective relative cell viability was calculated. As a result, as shown in Table 7, with introduction of molecules that produce hsa-miR-7, 24, 28, 95, 132, 134, 142-3p, 147, 182*, 183, 192, 197, 211, 222, 299-3p, 329, 337, 342, 362, 368, 376a, 376b, 377, 380-5p, 422b, 424, 431, 432*, 452, 452*, 455, 483, 485-5p, 489, 491, 493-5p, 494, 505, 506, 509, 510, 517a, and 517c, decreases of 50% or more in viable cell activity were observed. With introduction of molecules that produce hsa-miR-34c, 181a, and 124a, which are known to be associated with cancers, a decrease of 50% or more in cell viability was observed.

TABLE 7

| introduced miRNA | cell viability (Day 3) | cell viability (Day 5) |
|---|---|---|
| miR-362 | 0.17 | 0.06 |
| miR-491 | 0.29 | 0.14 |
| miR-132 | 0.31 | 0.30 |
| miR-299-3p | 0.34 | 0.15 |
| miR-376a | 0.34 | 0.27 |
| miR-483 | 0.34 | 0.20 |
| miR-493-5p | 0.35 | 0.18 |
| miR-368 | 0.37 | 0.42 |
| miR-124a | 0.38 | 0.18 |
| miR-181a | 0.38 | 0.27 |
| miR-376b | 0.38 | 0.25 |
| miR-377 | 0.39 | 0.65 |
| miR-432* | 0.41 | 0.24 |
| miR-342 | 0.43 | 0.18 |
| miR-197 | 0.43 | 0.08 |
| miR-494 | 0.45 | 0.21 |
| miR-183 | 0.46 | 0.50 |
| miR-211 | 0.47 | 0.45 |
| miR-489 | 0.48 | 0.23 |
| miR-424 | 0.49 | 0.44 |
| miR-28 | 0.50 | 0.47 |
| miR-95 | 0.50 | 0.28 |
| miR-517c | 0.51 | 0.33 |
| miR-452* | 0.52 | 0.25 |
| miR-192 | 0.53 | 0.46 |
| miR-7 | 0.53 | 0.42 |
| miR-380-5p | 0.55 | 0.37 |
| miR-182* | 0.55 | 0.41 |
| miR-142-3p | 0.55 | 0.37 |
| miR-329 | 0.56 | 0.42 |
| miR-506 | 0.56 | 0.39 |
| miR-452 | 0.57 | 0.28 |
| miR-505 | 0.57 | 0.46 |
| miR-422b | 0.57 | 0.42 |
| miR-147 | 0.58 | 0.36 |
| miR-455 | 0.62 | 0.38 |
| miR-510 | 0.63 | 0.49 |
| miR-509 | 0.65 | 0.46 |
| miR-222 | 0.67 | 0.45 |
| miR-34c | 0.68 | 0.45 |
| miR-431 | 0.69 | 0.29 |
| miR-134 | 0.71 | 0.42 |
| miR-485 | 0.72 | 0.44 |
| miR-337 | 0.75 | 0.47 |
| miR-24 | 0.76 | 0.50 |
| miR-517a | 0.84 | 0.43 |

Antisense oligonucleotides that are complementary to miRNAs were introduced into DLD-1 to obtain a final concentration of 50 nM using a lipofection method. As the antisense oligonucleotides that are complementary to the miRNAs, Anti-miR™miRNA Inhibitors of hsa-miR-197 (SEQ ID NO:53) and 483 (SEQ ID NO:95) (manufactured by Ambion) were used. Anti-miR™miRNA Inhibitors-Negative Control#1 (hereinafter, referred to as anti-miR-negacon#1) (manufactured by Ambion) was also introduced into DLD-1, and this was used as the negative control. The lipofection was performed per the directions attached to the product.

Three days after introduction of the antisense oligonucleotides by the lipofection method, cell viability was measured using CellTiter-Glo™Luminescent Cell Viability Assay (manufactured by Promega) per the directions attached to the product. Taking the cell viability for the DLD-1 in the anti-miR-negacon#1 introduction plot as 1.0, respective relative cell viability was calculated. As a result, as shown in Table 8, with introduction of antisense oligonucleotides that are complementary to hsa-miR-197 and 483, decreases of 50% or more in cell viability were observed.

TABLE 8

| introduced antisense | cell viability (50 nM) |
|---|---|
| anti-miR-483 | 0.43 |
| anti-miR-197 | 0.44 |

### Example 2

Cell Viability and Apoptotic Activity in Ovarian Cancer-Derived Cell Line with miRNA Expressed Forcedly Therein

Pre-miR™miRNA Precursor Molecules (manufactured by Ambion) were introduced into an ovarian cancer-derived cell line, and the influences of the miRNAs produced from the molecules on cell viability and apoptotic activity were examined.

The A2780 human ovarian cancer-derived cell line (Nature, 295, 116-119 (1982); Science, 224, 994-996 (1984); Semin. Oncol., 11, 285-298 (1984); hereinafter to be referred to as A2780) was cultured with an RPMI1640 medium (manufactured by Invitrogen) containing 5% FBS (manufactured by JRH Biosciences) in a 37° C. 5% CO₂ concentration incubator.

The A2780 was sown to a 96-well plate at 2500 cells per well, and cultured with an RPMI medium containing 10% FBS overnight. One day later, Pre-miR™ miRNA Precursor Molecules that generate hsa-miR-1 (SEQ ID NO:1), 7 (SEQ ID NO:3), 15a, 15b, 16, 18a (SEQ ID NO:5), 18a* (SEQ ID NO:7), 18b (SEQ ID NO:6), 28 (SEQ ID NO:17), 34b, 95 (SEQ ID NO:24), 98 (SEQ ID NO:25), 124a, 133a (SEQ ID NO:31), 133b (SEQ ID NO:32), 143, 145, 147 (SEQ ID NO:37), 181c (SEQ ID NO:39), 192 (SEQ ID NO:44), 193a (SEQ ID NO:46), 193b (SEQ ID NO:47), 195 (SEQ ID NO:48), 196a (SEQ ID NO:51), 196b (SEQ ID NO:52), 197 (SEQ ID NO:53), 199a* (SEQ ID NO:54), 202 (SEQ ID NO:56), 204 (SEQ ID NO:57), 211 (SEQ ID NO:58), 212 (SEQ ID NO:30), 215 (SEQ ID NO:45), 299-3p (SEQ ID NO:62), 324-3p (SEQ ID NO:66), 324-5p (SEQ ID NO:67), 337 (SEQ ID NO:70), 342 (SEQ ID NO:72), 346 (SEQ ID NO:73), 362 (SEQ ID NO:74), 368 (SEQ ID NO:75), 376a (SEQ ID NO:77), 376b (SEQ ID NO:78), 378 (SEQ ID NO:80), 380-5p (SEQ ID NO:81), 382 (SEQ ID NO:82), 422a (SEQ ID NO:84), 422b (SEQ ID NO:85), 424 (SEQ ID NO:49), 432* (SEQ ID NO:88), 451 (SEQ ID NO:91), 452 (SEQ ID NO:92), 452* (SEQ ID NO:93), 455 (SEQ ID NO:94), 483 (SEQ ID NO:95), 489 (SEQ ID NO:98), 491 (SEQ ID NO:99), 493-5p (SEQ ID NO:100), 494 (SEQ ID NO:101), 497 (SEQ ID NO:50), 501 (SEQ ID NO:102), 504 (SEQ ID NO:103), 506 (SEQ ID NO:105), 511 (SEQ ID NO:109), 512-5p (SEQ ID NO:111), 517a (SEQ ID NO:115), 517c (SEQ ID NO:116), 518b (SEQ ID NO:118), 518c (SEQ ID NO:119), 518c* (SEQ ID NO:121), 518d (SEQ ID NO:120), 527 (SEQ ID NO:129), let-7a, let-7b, let-7d, let-7d, let-7e, let-7f, let-7g, and let-7i were introduced into the A2780 to obtain a final concentration of 5 nM or 25 nM using a lipofection method, specifically Lipofectamine2000 (manufactured by Invitrogen). miR-negacon#2 (manufactured by Ambion) was also introduced into A2780, and this was used as the negative control. The lipofection was performed per the directions attached to the product.

Three days after introduction of the molecules by the lipofection method, cell viability was measured using CellTiter-Glo™Luminescent Cell Viability Assay (manufactured by Promega) per the directions attached to the product. Taking the cell viability for the A2780 in the control plot (Lipofectamine2000 only) as 1.0, respective relative cell viability was calculated. As a result, as shown in Table 9 (Table 9-1 and Table 9-2), with introduction of molecules that produce hsa-miR-1, 7, 18a, 18a*, 18b, 28, 95, 98, 133a, 133b, 147, 181c, 192, 193a, 193b, 195, 196a, 196b, 197, 199a*, 202, 204, 211, 212, 215, 299-3p, 324-3p, 324-5p, 342, 346, 362, 368, 376a, 376b, 378, 380-5p, 382, 422a, 422b, 424, 432*, 451, 452, 452*, 455, 483, 489, 491, 493-5p, 494, 497, 501, 504, 506, 511, 512-5p, 517a, 517c, 518b, 518c, 518c*, 518d, and 527, decreases of 40% or more in viable cell activity were observed. With introduction of molecules that produce hsa-miR-15a, 15b, 16, 34b, 124a, 143, 145, let-7a, let-7b, let-7d, let-7e, let-7f, let-7g, and let-7i, which are known to be associated with cancers, decreases of 40% or more in cell viability were observed.

TABLE 9

| Introduced miRNA | cell viability (25 nM) | cell viability (5 nM) |
|---|---|---|
| miR-124a | 0.13 | 0.24 |
| miR-192 | 0.21 | 0.32 |
| miR-193b | 0.22 | 0.35 |
| miR-193a | 0.23 | 0.33 |
| miR-491 | 0.25 | 0.41 |
| miR-506 | 0.26 | 0.35 |
| miR-195 | 0.29 | 0.44 |
| miR-497 | 0.29 | 0.37 |
| miR-34b | 0.29 | 0.36 |
| miR-493-5p | 0.30 | 0.47 |
| miR-196b | 0.30 | 0.43 |
| miR-197 | 0.31 | 0.43 |
| miR-489 | 0.31 | 0.48 |
| miR-452 | 0.31 | 0.45 |
| miR-147 | 0.32 | 0.40 |
| miR-324-3p | 0.32 | 0.50 |
| miR-15b | 0.32 | 0.42 |
| miR-518d | 0.32 | 0.40 |
| miR-215 | 0.32 | 0.33 |
| miR-378 | 0.33 | 0.48 |
| miR-518b | 0.33 | 0.37 |
| miR-196a | 0.34 | 0.48 |
| miR-517c | 0.34 | 0.72 |
| miR-517a | 0.34 | 0.58 |
| miR-432* | 0.34 | 0.51 |
| miR-7 | 0.34 | 0.44 |
| miR-376b | 0.35 | 0.53 |
| miR-18a* | 0.35 | 0.58 |
| let-7b | 0.36 | 0.49 |
| miR-346 | 0.36 | 0.49 |
| miR-527 | 0.36 | 0.64 |
| miR-518c | 0.37 | 0.46 |
| let-7f | 0.37 | 0.49 |
| let-7g | 0.37 | 0.49 |
| let-7i | 0.38 | 0.57 |
| miR-98 | 0.39 | 0.51 |
| miR-15a | 0.40 | 0.54 |
| miR-299-3p | 0.41 | 0.70 |
| miR-511 | 0.42 | 0.59 |
| miR-212 | 0.42 | 0.64 |
| miR-424 | 0.42 | 0.61 |
| miR-483 | 0.43 | 0.62 |
| miR-28 | 0.44 | 0.53 |
| let-7d | 0.44 | 0.57 |
| let-7e | 0.45 | 0.59 |
| let-7a | 0.46 | 0.68 |
| miR-211 | 0.46 | 0.61 |
| miR-422a | 0.46 | 0.65 |
| miR-324-5p | 0.46 | 0.68 |
| miR-504 | 0.47 | 0.61 |
| miR-204 | 0.47 | 0.66 |
| miR-368 | 0.48 | 0.59 |
| miR-199a* | 0.48 | 0.73 |
| miR-16 | 0.48 | 0.37 |
| miR-362 | 0.49 | 0.76 |
| miR-376a | 0.49 | 0.62 |
| miR-451 | 0.49 | 0.65 |
| miR-202 | 0.49 | 0.70 |
| miR-18a | 0.49 | 0.70 |
| miR-501 | 0.50 | 0.69 |
| miR-133a | 0.51 | 0.78 |
| miR-18b | 0.51 | 0.69 |
| miR-494 | 0.52 | 0.71 |
| miR-422b | 0.52 | 0.66 |
| miR-518c* | 0.52 | 0.78 |
| miR-512-5p | 0.52 | 0.72 |
| miR-382 | 0.53 | 0.77 |
| miR-342 | 0.53 | 0.78 |
| miR-95 | 0.54 | 0.79 |
| miR-181c | 0.55 | 0.72 |
| miR-380-5p | 0.56 | 0.53 |
| miR-145 | 0.57 | 0.86 |
| miR-1 | 0.58 | 0.78 |
| miR-452* | 0.58 | 0.79 |
| miR-143 | 0.59 | 0.75 |
| miR-133b | 0.59 | 0.75 |
| miR-455 | 0.60 | 0.76 |
| miR-negacon#2 | 0.79 | 0.96 |

Two days after introduction of the molecules (final concentration 25 nM) by the lipofection method, caspase 3/7 activity was measured using Caspase-Glo®3/7 assay (manufactured by Promega) per the directions attached to the product. Taking the caspase 3/7 activity value for the A2780 in the control plot (Lipofectamine2000 only) as 1.0, respective relative caspase 3/7 activity values were calculated. As a result, as shown in Table 10, with introduction of molecules that produce hsa-miR-18a*, 133a, 193a, 193b, 197, 199a*, 212, 324-3p, 337, 432*, 452*, 489, 491, 493-5p, 512-5p, 517a, 517c, and 527, increases of 50% or more in caspase 3/7 activity were observed. With introduction of a molecule that produces hsa-miR-124a, which is known to be associated with cancers, an increase of 50% or more in caspase 3/7 activity was observed.

TABLE 10

| introduced miRNA | caspase 3/7 activity |
|---|---|
| miR-527 | 2.88 |
| miR-193b | 2.70 |
| miR-193a | 2.63 |
| miR-199a* | 2.18 |
| miR-517c | 2.12 |
| miR-517a | 2.02 |
| miR-512-5p | 1.98 |
| miR-18a* | 1.96 |
| miR-489 | 1.91 |
| miR-493-5p | 1.89 |
| miR-432* | 1.84 |
| miR-197 | 1.80 |
| miR-337 | 1.76 |
| miR-133a | 1.68 |
| miR-452* | 1.68 |
| miR-124a | 1.60 |
| miR-491 | 1.59 |
| miR-212 | 1.58 |
| miR-324-3p | 1.56 |
| miR-negacon#2 | 1.25 |

In another test, A2780 was sown to a 96-well plate at 2500 cells per well, and cultured in an RPMI medium containing 10% FBS overnight. One day later, Pre-miR™miRNA Precursor Molecules of hsa-miR-7 (SEQ ID NO:3), 15a, 15b, 18a* (SEQ ID NO:7), 24 (SEQ ID NO:11), 26a (SEQ ID NO:14), 26b (SEQ ID NO:15), 34a, 34b, 34c, 124a, 130a (SEQ ID NO:26), 130b (SEQ ID NO:27), 134 (SEQ ID NO:33), 147 (SEQ ID NO:37), 192 (SEQ ID NO:44), 193a (SEQ ID NO:46), 193b (SEQ ID NO:47), 196a (SEQ ID NO:51), 196b (SEQ ID NO:52), 197 (SEQ ID NO:53), 199a* (SEQ ID NO:54), 204 (SEQ ID NO:57), 206 (SEQ ID NO:2), 211 (SEQ ID NO:58), 212 (SEQ ID NO:30), 215 (SEQ ID NO:45), 301 (SEQ ID NO:28), 346 (SEQ ID NO:73), 368 (SEQ ID NO:75), 371 (SEQ ID NO:76), 376b (SEQ ID NO:78), 378 (SEQ ID NO:80), 422a (SEQ ID NO:84), 424 (SEQ ID NO:49), 431 (SEQ ID NO:87), 432* (SEQ ID NO:88), 433 (SEQ ID NO:89), 449 (SEQ ID NO:90), 452 (SEQ ID NO:92), 483 (SEQ ID NO:95), 485-5p (SEQ ID NO:96), 488 (SEQ ID NO:97), 489 (SEQ ID NO:98), 491 (SEQ ID NO:99), 493-5p (SEQ ID NO:100), 497 (SEQ ID NO:50), 504 (SEQ ID NO:103), 505 (SEQ ID NO:104), 506 (SEQ ID NO:105), 507 (SEQ ID NO:106), 510 (SEQ ID NO:108), 511 (SEQ ID NO:109), 512-5p (SEQ ID NO:111), 513 (SEQ ID NO:112), 517a (SEQ ID NO:115), 517c (SEQ ID NO:116), 518b (SEQ ID NO:118), 518c (SEQ ID NO:119), 518d (SEQ ID NO:120), 526a (SEQ ID NO:128), 527 (SEQ ID NO:129), let-7b, let-7c, and let-7i were introduced into the A2780 to obtain a final concentration of 50 nM using a lipofection method, specifically Lipofectamine2000 (manufactured by Invitrogen). miR-negacon#2 (manufactured by Ambion) was also introduced into A2780, and this was used as the negative control. The lipofection was performed per the directions attached to the product.

Three or five days after introduction of the molecules by the lipofection method, cell viability was measured using CellTiter-Glo™Luminescent Cell Viability Assay (manufactured by Promega) per the directions attached to the product. Taking the cell viability for the A2780 in the miR-negacon#2 introduction plot as 1.0, respective relative cell viability was calculated. As a result, as shown in Table 11 (Table 11-1 and Table 11-2), with introduction of molecules that produce hsa-miR-7, 18a*, 24, 26a, 26b, 134, 147, 192, 193a, 193b, 196a, 196b, 197, 199a*, 204, 206, 211, 212, 215, 346, 368, 371, 376b, 378, 422a, 424, 431, 432*, 433, 449, 452, 483, 485-5p, 488, 489, 491, 493-5p, 497, 504, 505, 506, 507, 510, 511, 512-5p, 513, 517a, 517c, 518b, 518c, 518d, 526a, and 527, decreases of 50% or more in cell viability were observed. With introduction of miRNA precursors of molecules that produce hsa-miR-15a, 15b, 34a, 34b, 34c, 124a, let-7b, let-7c, and let-7i, which are known to be associated with cancers, decreases of 50% or more in cell viability were observed. Conversely, with introduction of molecules that produce hsa-miR-130a, 130b, and 301, increases in cell viability were observed.

TABLE 11

| introduced miRNA | cell viability (Day 3) | cell viability (Day 5) |
|---|---|---|
| miR-124a | 0.28 | 0.09 |
| miR-517c | 0.29 | 0.20 |
| miR-193a | 0.30 | 0.45 |
| miR-506 | 0.32 | 0.36 |
| miR-199a* | 0.35 | 0.44 |
| miR-192 | 0.37 | 0.31 |
| miR-7 | 0.37 | 0.46 |
| miR-193b | 0.38 | 0.44 |
| miR-432* | 0.38 | 0.19 |
| miR-497 | 0.38 | 0.33 |
| miR-518b | 0.39 | 0.17 |
| miR-34b | 0.39 | 0.29 |
| miR-527 | 0.39 | 0.40 |
| miR-197 | 0.39 | 0.20 |
| miR-510 | 0.41 | 0.48 |
| miR-15b | 0.41 | 0.73 |
| miR-518c | 0.41 | 0.18 |
| miR-505 | 0.42 | 0.53 |
| miR-196b | 0.43 | 0.68 |
| miR-493-5p | 0.43 | 0.37 |
| miR-215 | 0.43 | 0.47 |
| miR-196a | 0.43 | 0.67 |
| miR-518d | 0.44 | 0.34 |
| miR-424 | 0.46 | 0.31 |
| miR-526a | 0.46 | 0.65 |
| miR-511 | 0.46 | 0.45 |
| miR-346 | 0.46 | 0.35 |
| miR-507 | 0.47 | 0.64 |
| miR-15a | 0.47 | 0.53 |
| miR-26a | 0.49 | 0.57 |
| miR-26b | 0.49 | 0.58 |
| miR-147 | 0.49 | 0.40 |
| miR-489 | 0.50 | 0.21 |
| miR-491 | 0.50 | 0.38 |
| let-7i | 0.50 | 0.47 |
| let-7c | 0.50 | 0.65 |
| miR-422a | 0.51 | 0.14 |
| let-7b | 0.51 | 0.48 |
| miR-488 | 0.51 | 0.44 |
| miR-18a* | 0.51 | 0.44 |
| miR-376b | 0.51 | 0.48 |
| miR-378 | 0.52 | 0.31 |
| miR-134 | 0.53 | 0.45 |
| miR-517a | 0.54 | 0.39 |
| miR-512-5p | 0.57 | 0.48 |
| miR-431 | 0.58 | 0.39 |
| miR-368 | 0.59 | 0.50 |
| miR-433 | 0.59 | 0.49 |
| miR-206 | 0.60 | 0.49 |
| miR-24 | 0.60 | 0.46 |
| miR-449 | 0.61 | 0.26 |
| miR-212 | 0.62 | 0.45 |
| miR-513 | 0.62 | 0.39 |
| miR-485 | 0.63 | 0.36 |
| miR-483 | 0.64 | 0.42 |
| miR-504 | 0.64 | 0.33 |
| miR-452 | 0.65 | 0.42 |
| miR-34a | 0.66 | 0.42 |
| miR-211 | 0.68 | 0.41 |
| miR-34c | 0.73 | 0.50 |
| miR-204 | 0.74 | 0.41 |
| miR-371 | 0.97 | 0.49 |
| miR-130b | 1.62 | 0.99 |
| miR-130a | 1.73 | 1.18 |
| miR-301 | 2.18 | 1.23 |

Antisense oligonucleotides that are complementary to miRNAs were introduced into A2780 to obtain a final concentration of 50 nM using a lipofection method. As the antisense oligonucleotides that are complementary to the miRNAs, Anti-miR™miRNA Inhibitors (manufactured by Ambion) of hsa-miR-10a (SEQ ID NO:4), 20b (SEQ ID NO:8), 25 (SEQ ID NO:12), 27b (SEQ ID NO:16), 29a (SEQ ID NO:18), 29c (SEQ ID NO:19), 30b (SEQ ID NO:20), 30c (SEQ ID NO:21), 32 (SEQ ID NO:13), 93 (SEQ ID NO:22), 106a (SEQ ID NO:9), 106b (SEQ ID NO:10), 137 (SEQ ID NO:34), 146a (SEQ ID NO:36), 149 (SEQ ID NO:38), 191, 195 (SEQ ID NO:48), 200a (SEQ ID NO:55), 204 (SEQ ID NO:57), 205 (SEQ ID NO:59), 208 (SEQ ID NO:60), 212 (SEQ ID NO:30), 215 (SEQ ID NO:45), 301 (SEQ ID NO:28), 302a* (SEQ ID NO:64), 302b* (SEQ ID NO:65), 302d (SEQ ID NO:23), 324-3p (SEQ ID NO:66), 331 (SEQ ID NO:69), 340 (SEQ ID NO:71), 362 (SEQ ID NO:74), 412 (SEQ ID NO:83), 423 (SEQ ID NO:86), 451 (SEQ ID NO:91), 452 (SEQ ID NO:92), 483 (SEQ ID NO:95), 516-5p (SEQ ID NO:113), 518e (SEQ ID NO:122), 518f (SEQ ID NO:123), 520h (SEQ ID NO:124), 524* (SEQ ID NO:126), and 525 (SEQ ID NO:127) were used. Anti-miR-negacon#1 (manufactured by Ambion) was also introduced into A2780, and this was used as the negative control. The lipofection was performed per the directions attached to the product.

Three days after introduction of the antisense oligonucleotides by the lipofection method, cell viability was measured using CellTiter-Glo™luminescent Cell Viability Assay (manufactured by Promega) per the directions attached to the product. Taking the cell viability for the A2780 in the anti-miR-negacon#1 introduction plot as 1.0, respective relative cell viability was calculated. As a result, as shown in Table 12, with introduction of the antisense oligonucleotides that are complementary to hsa-miR-10a, 20b, 25, 27b, 29a, 29c, 30b, 30c, 32, 93, 106a, 106b, 137, 146a, 149, 191, 195, 200a, 204, 205, 208, 212, 215, 301, 302a*, 302b*, 302d, 324-3p, 331, 340, 362, 412, 423, 451, 452, 483, 516-5p, 518e, 518f, 520h, 524*, and 525, decreases of 40% or more in cell viability were observed.

TABLE 12

| introduced antisense | cell viability (50 nM) |
|---|---|
| anti-miR-29a | 0.39 |
| anti-miR-146 | 0.39 |
| anti-miR-200a | 0.41 |
| anti-miR-30c | 0.42 |
| anti-miR-412 | 0.43 |
| anti-miR-340 | 0.44 |
| anti-miR-25 | 0.44 |
| anti-miR-212 | 0.47 |
| anti-miR-331 | 0.48 |
| anti-miR-191 | 0.48 |
| anti-miR-451 | 0.48 |
| anti-miR-518f | 0.49 |
| anti-miR-518e | 0.49 |
| anti-miR-10a | 0.49 |
| anti-miR-204 | 0.49 |
| anti-miR-215 | 0.52 |
| anti-miR-106a | 0.52 |
| anti-miR-520h | 0.52 |
| anti-miR-208 | 0.53 |
| anti-miR-149 | 0.54 |
| anti-miR-205 | 0.54 |
| anti-miR-106b | 0.54 |
| anti-miR-302b* | 0.55 |
| anti-miR-483 | 0.55 |
| anti-miR-30b | 0.56 |
| anti-miR-362 | 0.57 |
| anti-miR-524* | 0.57 |
| anti-miR-27b | 0.57 |
| anti-miR-423 | 0.57 |
| anti-miR-93 | 0.58 |
| anti-miR-302a* | 0.58 |
| anti-miR-525 | 0.58 |
| anti-miR-324-3p | 0.58 |
| anti-miR-301 | 0.58 |
| anti-miR-302d | 0.59 |
| anti-miR-20b | 0.59 |
| anti-miR-195 | 0.59 |
| anti-miR-137 | 0.60 |
| anti-miR-516-5p | 0.60 |
| anti-miR-452 | 0.60 |
| anti-miR-29c | 0.60 |
| anti-miR-32 | 0.60 |

### Example 3

Cell Viability and Apoptotic Activity in Prostatic Cancer-Derived Cell Line with miRNA Expressed Forcedly Therein

Pre-miR™ miRNA Precursor Molecules (manufactured by Ambion) were introduced into a prostatic cancer-derived cell line, and the influences of the miRNAs produced from the molecules on cell viability and apoptotic activity were examined.

The PC-3 human prostatic cancer-derived cell line (hereinafter referred to as PC-3) was obtained from ATCC, and cultured with F-12 Kaighn's medium (manufactured by Invitrogen) containing 10% fetal bovine serum (FBS) (manufactured by JRH Biosciences) in an incubator at 37° C. and a 5% CO₂ concentration.

The PC-3 was sown to a 96-well plate at 3000 cells per well, and cultured in F-12 Kaighn's containing 10% FBS overnight. One day later, Pre-miR™ miRNA Precursor Molecules that produce hsa-miR-15a, 15b, 16, 18a* (SEQ ID NO:7), 34b, 124a, 133b (SEQ ID NO:32), 181b, 192 (SEQ ID NO:44), 195 (SEQ ID NO:48), 196b (SEQ ID NO:52), 324-3p (SEQ ID NO:66), 337 (SEQ ID NO:70), 362 (SEQ ID NO:74), 368 (SEQ ID NO:75), 376b (SEQ ID NO:78), 380-5p (SEQ ID NO:81), 432* (SEQ ID NO:88), 452 (SEQ ID NO:92), 452* (SEQ ID NO:93), 489 (SEQ ID NO:98), 491 (SEQ ID NO:99), 493-5p (SEQ ID NO:100), and 494 (SEQ ID NO:101) were introduced into the PC-3 to obtain a final concentration of 25 nM using a lipofection method, specifically Lipofectamine2000 (manufactured by Invitrogen). miR-negacon#2 (manufactured by Ambion) was also introduced into PC-3, and this was used as the negative control. The lipofection was performed per the directions attached to the product.

Three days after introduction of the molecules by the lipofection method, cell viability was measured using CellTiter-Glo™Luminescent Cell Viability Assay (manufactured by Promega) per the directions attached to the product. Taking the cell viability for the PC-3 in the control plot (Lipofectamine2000 only) as 1.0, respective relative cell viability was calculated. As a result, as shown in Table 13, with introduction of the molecules that produce hsa-miR-18a*, 133b, 192, 195, 196b, 324-3p, 337, 362, 368, 376b, 380⁻5p, 432*, 452, 452*, 489, 491, 493-5p, and 494, decreases of 40% or more in cell viability were observed. With the molecules that produce hsa-miR-15a, 15b, 16, 34b, 124a, and 181b, which are known to be associated with cancers, decreases of 40% or more in cell viability were observed.

TABLE 13

| introduced miRNA | cell viability (25 nM) |
|---|---|
| miR-362 | 0.06 |
| miR-181b | 0.19 |
| miR-491 | 0.20 |
| miR-494 | 0.36 |
| miR-489 | 0.42 |
| miR-432* | 0.42 |
| miR-452 | 0.44 |
| miR-493-5p | 0.45 |
| miR-124a | 0.47 |
| miR-376b | 0.51 |
| miR-15a | 0.51 |
| miR-133b | 0.52 |
| miR-16 | 0.53 |
| miR-368 | 0.53 |
| miR-15b | 0.54 |
| miR-195 | 0.55 |
| miR-452* | 0.56 |
| miR-380-5p | 0.56 |
| miR-196b | 0.56 |
| miR-192 | 0.57 |
| miR-337 | 0.57 |
| miR-34b | 0.57 |
| miR-324-3p | 0.58 |
| miR-18a* | 0.60 |
| miR-negacon#2 | 0.95 |

Two days after introduction of the molecules by the lipofection method, caspase 3/7 activity was measured using Caspase-Glo® 3/7 assay (manufactured by Promega) per the directions attached to the product. Taking the caspase 3/7 activity value for the PC-3 in the control plot (Lipofectamine2000 only) as 1.0, respective relative caspase 3/7 activity values were calculated. As a result, as shown in Table 14, with introduction of the molecules that produce hsa-miR-133b, 337, 362, 432*, 452*, 491, and 493-5p, increases of 50% or more in caspase 3/7 activity were observed. With introduction of the molecule that produces hsa-miR-181b, which is known to be associated with cancers, an increase of 50% or more in caspase 3/7 activity was observed.

TABLE 14

| introduced miRNA | caspase 3/7 activity |
|---|---|
| miR-491 | 5.12 |
| miR-362 | 3.27 |
| miR-181b | 2.76 |
| miR-432* | 2.38 |
| miR-452* | 2.13 |
| miR-133b | 1.77 |
| miR-493-5p | 1.65 |
| miR-337 | 1.62 |
| miR-negacon#2 | 1.11 |

### Example 4

BrdU Uptake Activity in Colorectal Cancer-Derived Cell Line (DLD-1) with miRNAs Expressed Forcedly Therein

Pre-miR™ miRNA Precursor Molecules (manufactured by Ambion) were introduced into DLD-1, and the influences of the miRNAs produced from the molecules on BrdU uptake activity were examined.

The DLD-1 was sown to a 96-well plate at 2500 cells per well, and cultured in an RPMI medium containing 10% FBS overnight. One day later, Pre-miR miRNA Precursor Molecules that produce hsa-miR-147 (SEQ ID NO:37), 342 (SEQ ID NO:72), 362 (SEQ ID NO:74), 376b (SEQ ID NO:78), 483 (SEQ ID NO:95), and 494 (SEQ ID NO:101) were introduced into the DLD-1 to obtain a final concentration of 25 nM using a lipofection method, specifically Lipofectamine2000 (manufactured by Invitrogen). miR-negacon#2 (manufactured by Ambion) was also introduced into DLD-1, and this was used as the negative control. The lipofection was performed per the directions attached to the product.

One day after introduction of the molecules by the lipofection method, BrdU uptake activity was measured using a BrdU labeling & detection kit III (manufactured by Roche) per the directions attached to the product. Taking the BrdU uptake activity for the DLD-1 in the miR-negacon#2 introduction plot as 1.0, respective relative BrdU uptake activities were calculated. As a result, as shown in Table 15, with introduction of the molecules that produce hsa-miR-147, 342, 362, 376b, 483, and 494, decreases of 40% or more in BrdU uptake activity were observed.

TABLE 15

| introduced miRNA | BrdU uptake activity |
|---|---|
| miR-342 | 0.22 |
| miR-362 | 0.48 |
| miR-147 | 0.52 |
| miR-376b | 0.56 |
| miR-494 | 0.57 |
| miR-483 | 0.59 |

BrdU Uptake Activity in Ovarian Cancer-Derived Cell Line (A2780) with miRNAs Expressed Forcedly Therein

Pre-miR™miRNA Precursor Molecules (manufactured by Ambion) were introduced into A2780, and the influences of the miRNAs produced from the molecules on BrdU uptake activity were examined.

A2780 was sown to a 96-well plate at 4000 cells per well, 25 and cultured in an RPMI medium containing 10% FBS overnight. One day later, the Pre-miR™ miRNA Precursor Molecules of hsa-miR-15a, 16, 124a, 147 (SEQ ID NO:37), 192 (SEQ ID NO:44), 193a (SEQ ID NO:46), 193b (SEQ ID NO:47), 215 (SEQ ID NO:45), 378 (SEQ ID NO:80), 432* (SEQ ID NO:88), 506 (SEQ ID NO:105), and 518c (SEQ ID NO:119) were introduced into the A2780 to obtain a final concentration of 25 nM using a lipofection method, specifically Lipofectamine2000 (manufactured by Invitrogen). miR-negacon#2 (manufactured by Ambion) was also introduced into A2780, and this was used as the negative control. The lipofection was performed per the directions attached to the product.

One day after introduction of the molecules by the lipofection method, BrdU uptake activity was measured using the BrdU labeling & detection kit III (manufactured by Roche) per the directions attached to the product. Taking the BrdU uptake activity for the A2780 in the miR-negacon#2 introduction plot as 1.0, respective relative BrdU uptake activities were calculated. As a result, as shown in Table 16, with introduction of the molecules that produce hsa-miR-hsa-miR-147, 192, 193a, 193b, 215, 378, 432*, 506, and 518c, decreases of 40% or more in BrdU uptake activity were observed. With introduction of the molecules that produce hsa-miR-15a, 16, and 124a, which are known to be associated with cancers, decreases of 40% or more in BrdU uptake activity were observed.

TABLE 16

| introduced miRNA | BrdU uptake activity |
|---|---|
| miR-124a | 0.32 |
| miR-518c | 0.37 |
| miR-506 | 0.42 |
| miR-15a | 0.45 |
| miR-192 | 0.48 |
| miR-193a | 0.48 |
| miR-215 | 0.49 |
| miR-193b | 0.51 |
| miR-16 | 0.52 |
| miR-432* | 0.54 |
| miR-378 | 0.54 |
| miR-147 | 0.56 |

### Example 5

Analysis of the Expression Level of Proteins Encoded by Target Genes for hsa-miR-337, 342, 432*, 491, and 518b

Molecules that produce hsa-miR-337, 342, 432*, 491, and 518b, which exhibited DLD-1 proliferation suppression or caspase activity in Example 1, were introduced into DLD-1, and changes in the amounts of proteins encoded by the target genes were examined.

Pre-miR™miRNA Precursor Molecules (manufactured by Ambion) that produce hsa-miR-16, 147, 337, 342, 432*, 491, and 518b were introduced into previously sown 1×10⁵ cells of DLD-1 to obtain a final concentration of 25 nM, using Lipofectamine2000. miR-negacon#2 was also introduced, and this was used as the negative control. Also, si-Bc1211, si-Birc5, and si-Bc191 (manufactured by QIAGEN) were introduced to obtain a final concentration of 25 nM, and these were used as the positive controls. Two days after the introduction, the cells were recovered in solution in an RIPA buffer [50 mM Tris-HCL (pH 8.0), 150 mM NaCl, 1% Nonident P-40, 0.5% sodium deoxycholate, 0.1% sodium dodecyl sulfate, 1% Protease Inhibitor Cocktail SetIII (manufactured by Calbiochem)]. After separation by a conventional method of SDS-PAGE, the amounts of the proteins of Bc1211, Birc5 and Bc191 were detected by a Western blotting method. For detection of Bc1211, an anti-Bc1211 antibody (manufactured by BD Biosciences) was used; for detection of Birc5, an anti-Birc5 antibody (manufactured by R&D Systems) was used; for detection of Bc191, an anti-Bc191 antibody (manufactured by Abnova) was used. Also detected was the amount of the alpha-tubulin protein; this was used as the control for the amount of sample supplied. To detect alpha-tubulin, an anti-alpha-tubulin antibody (manufactured by Sigma) was used.

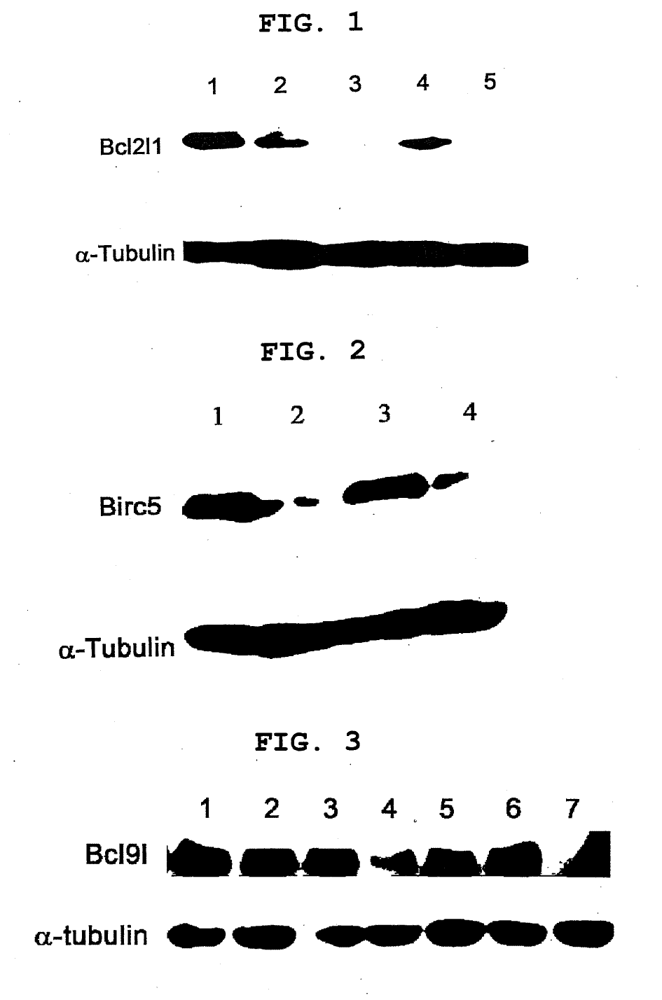

As a result, as shown in FIG. 1, the DLD-1 with hsa-miR-337, 342, and 491 expressed forcedly therein exhibited decreased amounts of the protein of Bc1211 compared with the negative control DLD-1, showing that the Bc1211 gene is the target gene for hsa-miR-337, 342, and 491, and has its expression regulated. As shown in FIG. 2, the DLD-1 with hsa-miR-337 expressed forcedly therein exhibited a decreased amount of the protein of Birc5 compared with the negative control DLD-1, showing that the Birc5 gene is the target gene for hsa-miR-337, and has its expression regulated. Furthermore, as shown in FIG. 3, the DLD-1 with hsa-miR-432* or 518b expressed forcedly therein exhibited decreased amounts of the protein of Bc191 compared with the negative control DLD-1, showing that the Bc191 gene is a target gene for hsa-miR-432* and 518b, and has its expression regulated.

### Example 6

Cell Viability and Apoptotic Activity in Colorectal Cancer-Derived Cell Line with miRNAs Expressed Forcedly Therein

DLD-1 was sown to a 96-well plate at 2500 cells per well, and cultured in an RPMI medium containing 10% FBS overnight. One day later, Pre-miR™miRNA Precursor Molecules that produce hsa-miR-493-3p (SEQ ID NO:2061), 542-3p (SEQ ID NO:2063), 571 (SEQ ID NO:2076), 579 (SEQ ID NO:2077), 625 (SEQ ID N0:2099), 630 (SEQ ID NO:2101), 634 (SEQ ID NO:2103), 650 (SEQ ID NO:2108), and 653 (SEQ ID NO:2110) were introduced into the DLD-1 to obtain a final concentration of 50 nM using a lipofection method, specifically Lipofectamine2000 (manufactured by Invitrogen). miR-negacon#2 (manufactured by Ambion) was also introduced into DLD-1, and this was used as the negative control. The lipofection was performed per the directions attached to the product.

Three days after introduction of the miRNA precursors by the lipofection method, cell viability was measured using CellTiter-Glo™Luminescent Cell Viability Assay (manufactured by Promega) per the directions attached to the product. Taking the cell viability for the DLD-1 in the miR-negacon#2 introduction plot as 1.0, respective relative cell viability was calculated. As a result, as shown in Table 17, with introduction of the molecules that produce hsa-miR-493-3p, 542-3p, 571, 579, 625, 630, 634, 650, and 653, decreases of 50% or more in cell viability were observed.

In another test, two days after introduction of the Pre-miR™ miRNA Precursor Molecules that produces hsa-miR-634 by the lipofection method, caspase 3/7 activity was measured using Caspase-Glo^{R} 3/7 assay (manufactured by Promega) per the directions attached to the product. Taking the caspase activity for the DLD-1 in the miR-negacon#2 introduction plot as 1.0, relative caspase activity was calculated. As a result, with introduction of the molecule that produces hsa-miR-634, an increase of 50% or more in caspase activity was observed.

TABLE 17

| introduced miRNA | cell viability (Day 3) |
|---|---|
| miR-634 | 0.37 |
| miR-653 | 0.41 |
| miR-650 | 0.43 |
| miR-493-3p | 0.44 |
| miR-542-3p | 0.45 |
| miR-571 | 0.45 |
| miR-625 | 0.46 |
| miR-630 | 0.48 |
| miR-579 | 0.49 |

Antisense oligonucleotides that are complementary to miRNAs were introduced into DLD-1 to obtain a final concentration of 50 nM using a lipofection method. As the antisense oligonucleotides that are complementary to the miRNAs, Anti-miR™ miRNA Inhibitors (manufactured by Ambion) of hsa-miR-411 (SEQ ID NO:2057), 425-5p (SEQ ID NO:2059), 449b (SEQ ID NO:917), 532 (SEQ ID NO:2062), 548b (SEQ ID NO:2066), 558 (SEQ ID NO:2072), 589 (SEQ ID NO:2079), 593 (SEQ ID NO:2081), 599 (SEQ ID NO:2083), 615 (SEQ ID NO:2093), 637 (SEQ ID NO:2104), 657 (SEQ ID NO:2112), 769-5p (SEQ ID NO:2114), and 92b (SEQ ID NO:819) were used. Anti-miR-negacon#1 (manufactured by Ambion) was also introduced into DLD-1, and this was used as the negative control. The lipofection was performed per the directions attached to the product.

Three days after introduction of the antisense oligonucleotides by the lipofection method, cell viability was measured using CellTiter-Glo™Luminescent Cell Viability Assay (manufactured by Promega) per the directions attached to the product. Taking the cell viability for the DLD-1 in the anti-miR-negacon#1 introduction plot as 1.0, respective relative cell viability was calculated. As a result, as shown in Table 18, with introduction of the antisense oligonucleotides complementary to hsa-miR-411, 425-5p, 449b, 532, 548b, 558, 589, 593, 599, 615, 637, 657, 769-5p, and 92b, decreases of 40% or more in cell viability were observed.

TABLE 18

| introduced antisense RNA | cell viability (Day 3) |
|---|---|
| anti-miR-593 | 0.52 |
| anti-miR-411 | 0.53 |
| anti-miR-589 | 0.55 |
| anti-miR-558 | 0.56 |
| anti-miR-92b | 0.57 |
| anti-miR-657 | 0.57 |
| anti-miR-548b | 0.57 |
| anti-miR-615 | 0.58 |
| anti-miR-532 | 0.58 |
| anti-miR-425-5p | 0.59 |
| anti-miR-599 | 0.59 |
| anti-miR-637 | 0.59 |
| anti-miR-769-5p | 0.60 |
| anti-miR-449b | 0.60 |

### Example 7

Cell Viability in Ovarian Cancer-Derived Cell Line with miRNAs Expressed Forcedly Therein

A2780 was sown to a 96-well plate at 2500 cells per well, and cultured in an RPMI medium containing 10% FBS overnight. One day later, Pre-miR™ miRNA Precursor Molecules that produce hsa-miR-421 (SEQ ID NO:2058), 454-5p (SEQ ID NO:2060), 542-3p (SEQ ID NO:2063), 545 (SEQ ID NO:2064), 548a (SEQ ID NO:2065), 548d (SEQ ID NO:2067), 550 (SEQ ID NO:2068), 551b (SEQ ID NO:2069), 552 (SEQ ID NO:2070), 553 (SEQ ID NO:2071), 557 (SEQ ID NO:925), 561 (SEQ ID NO:2073), 563 (SEQ ID NO:916), 565 (SEQ ID NO:2074), 570 (SEQ ID NO:2075), 571 (SEQ ID NO:2076), 584 (SEQ ID NO:2078), 591 (SEQ ID NO:2080), 595 (SEQ ID NO:2082), 600 (SEQ ID NO:2084), 601 (SEQ ID NO:2085), 604 (SEQ ID NO:2086), 605 (SEQ ID NO:2087), 606 (SEQ ID NO:2088), 609 (SEQ ID NO:2089), 610 (SEQ ID NO:2090), 611 (SEQ ID NO:2091), 613 (SEQ ID NO:803), 614 (SEQ ID NO:2092), 618 (SEQ ID NO:2096), 621 (SEQ ID NO:2097), 624 (SEQ ID NO:2098), 625 (SEQ ID NO:2099), 627 (SEQ ID NO:2100), 630 (SEQ ID NO:2101), 631 (SEQ ID NO:2102), 634 (SEQ ID NO:2103), 637 (SEQ ID NO:2104), 646 (SEQ ID NO:2105), 647 (SEQ ID NO:2106), 648 (SEQ ID NO:2107), 653 (SEQ ID NO:2110), 654 (SEQ ID NO:2111), and 661 (SEQ ID NO:2113) were introduced into the A2780 to obtain a final concentration of 50 nM using a lipofection method, specifically Lipofectamine2000 (manufactured by Invitrogen). miR-negacon#2 (manufactured by Ambion) was also introduced into A2780, and this was used as the negative control. The lipofection was performed per the directions attached to the product.

Three days after introduction of the molecules by the lipofection method, cell viability was measured using CellTiter-Glo™luminescent Cell Viability Assay (manufactured by Promega) per the directions attached to the product. Taking the cell viability for the A2780 in the miR-negacon#2 introduction plot as 1.0, respective relative cell viability was calculated. As a result, as shown in Table 19, with introduction of the molecules that produce hsa-miR-421, 454-5p, 542-3p, 545, 548a, 548d, 550, 551b, 552, 553, 557, 561, 563, 565, 570, 571, 584, 591, 595, 600, 601, 604, 605, 606, 609, 610, 611, 613, 614, 618, 621, 624, 625, 627, 630, 631, 634, 637, 646, 647, 648, 653, 654, and 661, decreases of 50% or more in cell viability were observed.

TABLE 19

| introduced miRNA | cell viability (Day 3) |
|---|---|
| miR-634 | 0.21 |
| miR-552 | 0.21 |
| miR-611 | 0.22 |
| miR-604 | 0.26 |
| miR-646 | 0.28 |
| miR-630 | 0.30 |
| miR-421 | 0.30 |
| miR-571 | 0.32 |
| miR-647 | 0.33 |
| miR-605 | 0.33 |
| miR-627 | 0.34 |
| miR-614 | 0.37 |
| miR-637 | 0.37 |
| miR-631 | 0.38 |
| miR-609 | 0.38 |
| miR-654 | 0.38 |
| miR-454-5p | 0.39 |
| miR-542-3p | 0.40 |
| miR-601 | 0.42 |
| miR-561 | 0.42 |
| miR-600 | 0.42 |
| miR-618 | 0.42 |
| miR-653 | 0.43 |
| miR-591 | 0.43 |
| miR-548a | 0.44 |
| miR-565 | 0.44 |
| miR-584 | 0.45 |
| miR-563 | 0.45 |
| miR-548d | 0.46 |
| miR-570 | 0.46 |
| miR-606 | 0.47 |
| miR-550 | 0.47 |
| miR-557 | 0.47 |
| miR-648 | 0.48 |
| miR-621 | 0.48 |
| miR-545 | 0.49 |
| miR-551b | 0.49 |
| miR-625 | 0.49 |
| miR-595 | 0.49 |
| miR-624 | 0.49 |
| miR-661 | 0.49 |
| miR-610 | 0.50 |
| miR-613 | 0.50 |
| miR-553 | 0.50 |

Antisense oligonucleotides that are complementary to miRNAs were introduced into A2780 to obtain a final concentration of 50 nM using a lipofection method. As the antisense oligonucleotides that are complementary to the miRNAs, Anti-miR™miRNA Inhibitors (manufactured by Ambion) of hsa-miR-611 (SEQ ID NO:2091), 616 (SEQ ID NO:2094), 617 (SEQ ID NO:2095), 651 (SEQ ID NO:2109), and 657 (SEQ ID NO:2112) were used. Anti-miR-negacon#1 (manufactured by Ambion) was also introduced into A2780, and this was used as the negative control. The lipofection was performed per the directions attached to the product.

Three days after introduction of the antisense oligonucleotides by the lipofection method, cell viability was measured using CellTiter-Glo™Luminescent Cell Viability Assay (manufactured by Promega) per the directions attached to the product. Taking the cell viability for the A2780 in the anti-miR-negacon#1 introduction plot as 1.0, respective relative cell viability was calculated. As a result, as shown in Table 20, with introduction of the antisense oligonucleotides that are complementary to hsa-miR-611, 616, 617, 651, and 657, decreases of 40% or more in cell viability were observed.

TABLE 20

| introduced antisense RNA | cell viability (Day 3) |
|---|---|
| anti-miR-617 | 0.58 |
| anti-miR-611 | 0.59 |
| anti-miR-651 | 0.59 |
| anti-miR-657 | 0.59 |
| anti-miR-616 | 0.60 |

### Example 8

Tumorigenesis by Transplantation of Colorectal Cancer-Derived Cell Line with miRNAs Expressed Forcedly Therein to Nude Mice

DLD-1 incorporating molecules that produce hsa-miR-147, 342, and 491, which exhibited DLD-1 proliferation suppression or caspase activity in Example 1, was transplanted to nude mice, and the influences of miRNAs on tumorigenesis were examined.

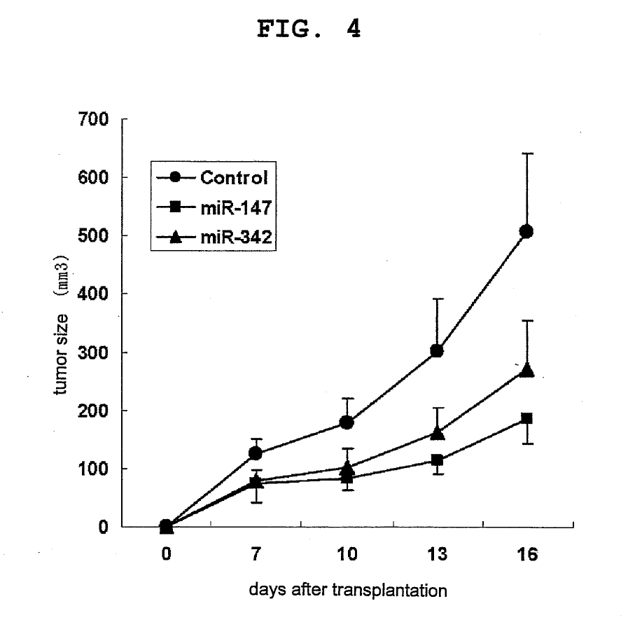

Pre-miR™ miRNA Precursor Molecules (manufactured by Ambion) that produce hsa-miR-147, 342, and 491 were introduced into previously sown 1×10⁶ cells of DLD-1 to obtain a final concentration of 50 nM using Lipofectoamine 2000. miR-negacon#2 was also introduced, and this was used as the negative control. One day after the introduction, cells were detached using 0.05% trypsin solution (manufactured by Gibco). The detached cells were mixed with Matrigel (manufactured by BD Bioscience), and 2.5×10⁵ cells were subcutaneously transplanted to the flanks of nude mice at 6 weeks of age. The major diameters and minor diameters of the tumors formed after 7, 10, 13, and 16 days of transplantation were measured. Tumor size was calculated as major diameterx(minor diameter)2×0.5. As shown in FIG. 4, with transplantation of the DLD-1 with hsa-miR-147 or 342 expressed forcedly therein, tumorigenesis was suppressed compared with the negative control.

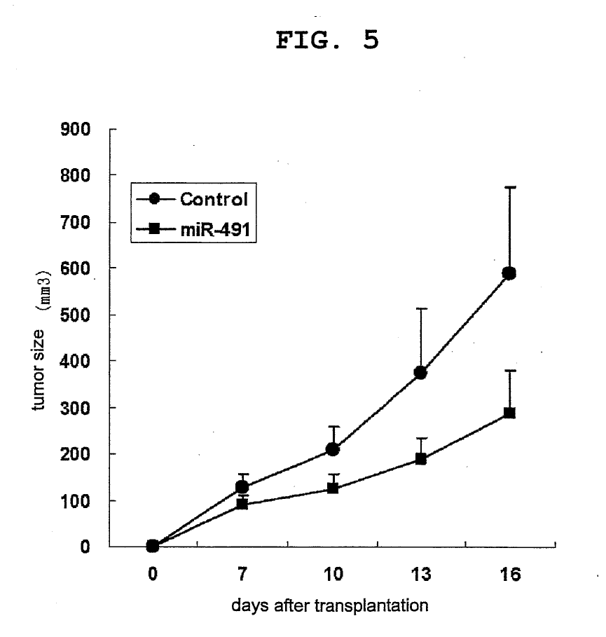

As shown in FIG. 5, with transplantation of the DLD-1 with hsa-miR-491 expressed forcedly therein, tumorigenesis was suppressed, compared with the negative control.

### INDUSTRIAL APPLICABILITY

An agent for suppressing or promoting cell proliferation, a diagnostic reagent or therapeutic drug for a disease resulting from a cell proliferation abnormality, an agent for inducing apoptosis, an agent for suppressing or promoting the expression of a target gene for a nucleic acid such as a miRNA, and a method of suppressing or promoting cell proliferation can be provided.

### Sequence Listing Free Text

SEQ ID NO: 1399-n is a, c, g or u

1.-32. (canceled)

33. A method of suppressing or promoting cell proliferation in a patient, which method comprises administering to the patient a nucleic acid selected from the group consisting of:
(a1) a nucleic acid consisting of any one of SEQ ID NO: 1-939, 2057-2132, 2212-2261, and 2315-2321;
(b1) a nucleic acid consisting of 17 to 28 sequential nucleotides of any one of SEQ ID NO: 1-939, 2057-2132, 2212-2261, and 2315-2321;
(c1) a nucleic acid having 90% or more identity to one of SEQ ID NO: 1-939, 2057-2132, 2212-2261, and 2315-2321;
(d1) a nucleic acid that hybridizes under stringent conditions with a complementary strand of any one of SEQ ID NO: 1-939, 2057-2132, 2212-2261 and 2315-2321;
(e1) a nucleic acid comprising the 2nd to 8th nucleotides of any one of SEQ ID NO: 1-939, 2057-2132, 2212-2261, and 2315-2321;
(f1) a nucleic acid consisting of any one of SEQ ID NO: 940-2056, 2133-2211, 2262-2314, and 2322-2328;
(g1) a nucleic acid having 90% or more identity to any one of SEQ ID NO: 940-2056, 2133-2211, 2262-2314, and 2322-2328; and
(h1) a nucleic acid that hybridizes under stringent conditions with a complementary strand of any one of SEQ ID NO: 940-2056, 2133-2211, 2262-2314 and 2322-2328, thereby suppressing or promoting cell proliferation in the patient.

34. The method of claim 33, wherein the nucleic acid is microRNA or microRNA precursor.

35. A method of suppressing or promoting cell proliferation in a patient, which method comprises administering to a patient:
(a2) a nucleic acid complementary to a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(b2) a vector that expresses a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(c2) a vector that expresses a nucleic acid complementary to a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(d2) a substance that suppresses or promotes the expression of a gene having a target nucleotide sequence for a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(e2) a nucleic acid that suppresses or promotes the expression of a gene having a target nucleotide sequence for a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(f2) a siRNA that that suppresses or promotes the expression of a gene having a target nucleotide sequence for a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328; or
(g2) a vector that expresses the nucleic acid of (e2),
thereby suppressing or promoting cell proliferation in the patient.

36. A method of treating a disease resulting from a cell proliferation abnormality in a patient, which method comprises administering to a patient with a disease resulting from a cell proliferation abnormality an effective amount of:
(a3) a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (e1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(b3) a nucleic acid complementary to the nucleic acid of (a3);
(c3) a vector that expresses the nucleic acid of (a3);
(d3) a vector that expresses the nucleic acid of (b3);
(e3) a substance that suppresses or promotes the expression of a gene having a target nucleotide sequence for the nucleic acid of (a3);
(f3) a nucleic acid that suppresses or promotes the expression of a gene having a target nucleotide sequence for the nucleic acid of (a3); or
(g3) a vector that expresses the nucleic acid of (f3), thereby treating the disease in the patient.

37. The method of claim 35, wherein the disease is cancer.

38. A method of treating a disease resulting from a cell proliferation abnormality in a patient, which method comprises administering to a patient with a disease resulting from a cell proliferation abnormality an effective amount of:
(a4) a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(b4) a nucleic acid complementary to the nucleic acid of (a4); or
(c4) a nucleic acid that suppresses or promotes the expression of a gene having a target nucleotide sequence for the nucleic acid of (a4), thereby treating the disease in the patient.

39. The method of claim 38, wherein the disease is cancer.

40. A method of diagnosing a disease resulting from a cell proliferation abnormality in a patient, which method comprises:
(ia) obtaining a sample from a patient,
(iia) measuring the expression level of a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328, and
(iiia) comparing the expression level obtained in (iia) with the expression level in normal tissue, or
(ib) obtaining a sample from a patient, and
(iib) detecting a mutation of the nucleic acid or the genome that encodes the nucleic acid,
thereby diagnosing a disease resulting from a cell proliferation abnormality in the patient.

41. The method of claim 40, wherein the disease is cancer.

42. A method of suppressing or promoting expression of a target gene in a patient comprising administering to a patient:
(a5) a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328;
(b5) a nucleic acid complementary the nucleic acid of (a5);
(c5) a vector that expresses the nucleic acid of (a5); or
(d5) a vector that expresses the nucleic acid of (b5),
thereby suppressing or promoting expression of a target gene in the patient.

43. A method of screening for an agent that promotes or suppresses cell proliferation, which method comprises:
(i) measuring a first expression level of a nucleic acid selected from the group consisting of (a1) a nucleic acid consisting of any one of SEQ ID NO: 1-939, 2057-2132, 2212-2261, and 2315-2321; (b1) a nucleic acid consisting of 17 to 28 sequential nucleotides of any one of SEQ ID NO: 1-939, 2057-2132, 2212-2261, and 2315-2321; (c1) a nucleic acid having 90% or more identity to one of SEQ ID NO: 1-939, 2057-2132, 2212-2261, and 2315-2321; (d1) a nucleic acid that hybridizes under stringent conditions with a complementary strand of any one of SEQ ID NO: 1-939, 2057-2132, 2212-2261 and 2315-2321; (e1) a nucleic acid comprising the 2nd to 8th nucleotides of any one of SEQ ID NO: 1-939, 2057-2132, 2212-2261, and 2315-2321; (f1) a nucleic acid consisting of any one of SEQ ID NO: 940-2056, 2133-2211, 2262-2314, and 2322-2328; (g1) a nucleic acid having 90% or more identity to any one of SEQ ID NO: 940-2056, 2133-2211, 2262-2314, and 2322-2328; and (h1) a nucleic acid that hybridizes under stringent conditions with a complementary strand of any one of SEQ ID NO: 940-2056, 2133-2211, 2262-2314 and 2322-2328 in a cell that expresses the nucleic acid;
(ii) bringing a test substance into contact with the cell of (i),
(iii) measuring a second expression level of the same nucleic acid; and
(iv) comparing the first and second expression levels, wherein a change in the first and second expression levels indicates that the test substance is an agent that promotes or suppresses cell proliferation.
